(19) United States
(12) Patent Application Publication
(10) Pub. No.: US 2026/0078131 A1
(43) Pub. Date: Mar. 19, 2026

(54) PYRROLO-PYRIDAZINE INHIBITORS OF JAK2

(71) Applicant: Ajax Therapeutics, Inc., New York, NY (US)

(72) Inventors: Craig E. Masse, Cambridge, MA (US); Jiayi Xu, Marlboro, NJ (US); Phani Ghanakota, Edison, NJ (US); Kevin Robert DeMarco, Cotati, CA (US); Susanta Haldar, Hyderabad (IN); Alice Hooper, Brooklyn, NY (US)

(21) Appl. No.: 19/320,373

(22) Filed: Sep. 5, 2025

Related U.S. Application Data
(60) Provisional application No. 63/831,517, filed on Jun. 27, 2025.

(30) Foreign Application Priority Data
Sep. 6, 2024 (IN) 202411067506

(51) Int. Cl. C07D 487/14; C07B 59/00; C07D 498/22; C07D 519/00

(52) U.S. Cl. CPC C07D 487/14; C07B 59/002; C07D 498/22; C07D 519/00

(57) ABSTRACT

The present disclosure provides pyrrolo-pyridazine compounds and compositions thereof useful for inhibiting JAK2. In some embodiments, the present disclosure provides a compound of formula I:

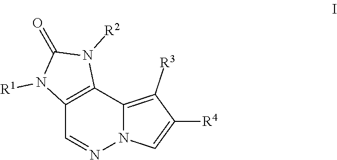

or a pharmaceutically acceptable salt thereof, wherein R¹, R², R³, and R⁴ are as defined in classes and subclasses herein, both singly and in combination.

### CROSS-REFERENCE TO RELATED APPLICATIONS

This application claims priority to and benefit of Indian Appl. No. 202411067506, filed Sep. 6, 2024, and U.S. Appl. No. 63/831,517, filed Jun. 27, 2025, the entire contents of each of which are hereby incorporated by reference.

### BACKGROUND

Janus kinases (JAK1, JAK2, JAK3, and TYK2) are a family of non-receptor tyrosine kinases that may have important roles in the regulation of hematopoeisis, the immune system, and cellular metabolism. JAKs can interact with certain cytokine receptors and can couple cytokine binding to cytoplasmic signaling cascades, including the signal transducers and activators of transcription (STAT) pathway. In addition to a canonical tyrosine kinase domain (JH1) located in the C-terminal region, JAK proteins also contain a pseudokinase domain (JH2).

Both JH1 and JH2 contain an ATP binding site, but catalytic activity is believed to come predominantly from JH1, as JH2 lacks essential residues for phosphorylation catalysis. However, JH2 mediates critical regulatory functions in JAKs and is believed to primarily serve to inhibit basal JAK2 activity. Mutations to JH2 may therefore, in some instances, lead to kinase hyperactivity. For example, a V617F mutation to the JH2 domain of JAK2 may promote constitutive activation of the JAK-STAT pathway, and may play a causative role in various myeloproliferative disorders. The V617F mutation is found 95% of patients with polycythemia vera, as well as ˜60% of patients with essential thrombocythemia and primary myelofibrosis. JAK2 hyperactivation is also associated with several leukemias and lymphomas. Disruption of ATP binding in JH2 inhibits the hyperactivity of JAK2 V617F and other pathogenic JAK2 mutants (Hammaren, H. et. Al., Proc. Natl. Acad. Sci., 2015, 112 (15), 4642-4647).

A modulator of JAK2 kinase activity (e.g., a JAK2 inhibitor disclosed herein) may be effective in treating a disease associated with JAK-STAT hyperactivity. Therefore, a JAK2 modulator capable of reducing JH1 activity and/or promoting JH2 regulatory function (e.g., by inhibiting ATP binding to JH2) could be useful in restoring or treating aberrant JAK2-mediated signaling, and in treating diseases associated therewith.

### SUMMARY

The present disclosure provides compounds useful for inhibiting JAK2. In some embodiments, provided compounds are useful for, among other things, treating and/or preventing diseases, disorders, or conditions associated with JAK2.

In some embodiments, the present disclosure provides a compound of Formula I:

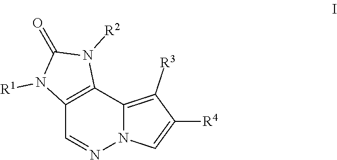

or a pharmaceutically acceptable salt thereof, wherein R¹, R², R³, and R⁴ are as defined in classes and subclasses herein, both singly and in combination.

In some embodiments, the present disclosure provides a compound represented by formula II-1 or II-2:

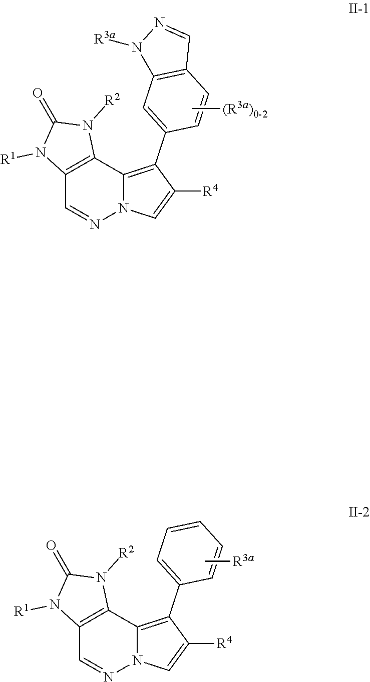

or a pharmaceutically acceptable salt thereof, wherein R¹, R², R^{3a}, and R⁴ are as defined in classes and subclasses herein, both singly and in combination.

In some embodiments, the present disclosure provides a compound represented by formula II-1:

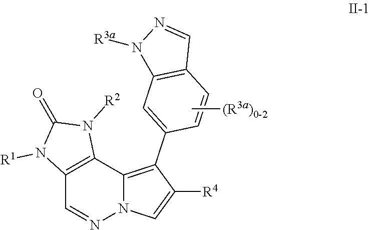

or a pharmaceutically acceptable salt thereof, wherein R¹, R², R^{3a}, and R⁴ are as defined in classes and subclasses herein, both singly and in combination.

In some embodiments, the present disclosure provides a compound represented by formula II-2:

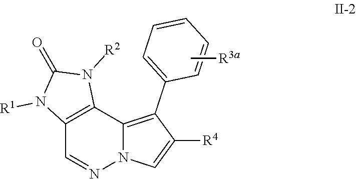

or a pharmaceutically acceptable salt thereof, wherein R¹, R², R^{3a}, and R⁴ are as defined in classes and subclasses herein, both singly and in combination.

In some embodiments, the present disclosure provides a compound represented by formula II-3:

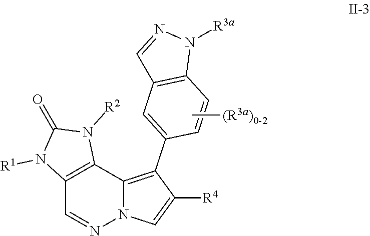

or a pharmaceutically acceptable salt thereof, wherein R¹, R², R^{3a}, and R⁴ are as defined in classes and subclasses herein, both singly and in combination.

In some embodiments, the present disclosure provides a compound represented by formula II-1, II-2, or II-3, or a pharmaceutically acceptable salt thereof.

In some embodiments, the present disclosure provides a compound represented by formula III:

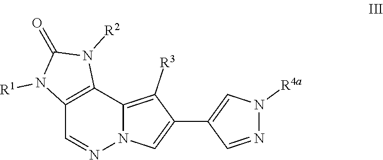

or a pharmaceutically acceptable salt thereof, wherein R¹, R², R³, and R^{4a} are as defined in classes and subclasses herein, both singly and in combination.

In some embodiments, the present disclosure provides a compound represented by formula IV-1 or IV-2:

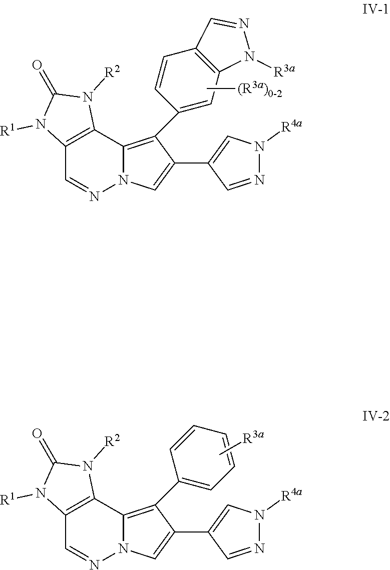

or a pharmaceutically acceptable salt thereof, wherein R¹, R², R^{3a}, and R^{4a} are as defined in classes and subclasses herein, both singly and in combination.

In some embodiments, the present disclosure provides a compound represented by formula IV-1:

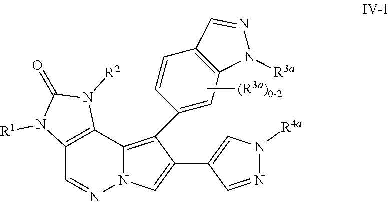

or a pharmaceutically acceptable salt thereof, wherein R¹, R², R^{3a}, and R^{4a} are as defined in classes and subclasses herein, both singly and in combination.

In some embodiments, the present disclosure provides a compound represented by formula IV-2:

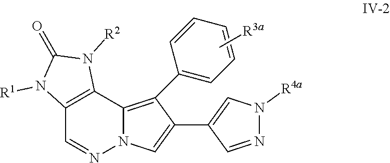

or a pharmaceutically acceptable salt thereof, wherein R¹, R², R^{3a}, and R^{4a} are as defined in classes and subclasses herein, both singly and in combination.

In some embodiments, the present disclosure provides a compound represented by formula IV-3:

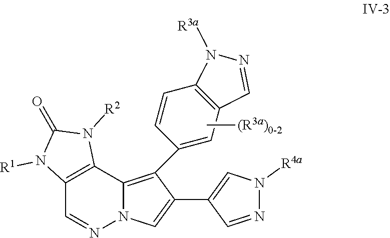

or a pharmaceutically acceptable salt thereof, wherein R¹, R², R^{3a}, and R^{4a} are as defined in classes and subclasses herein, both singly and in combination.

In some embodiments, the present disclosure provides a compound represented by formula IV-1, IV-2, or IV-3, or a pharmaceutically acceptable salt thereof.

In some embodiments, the present disclosure provides a compound represented by formula V-1:

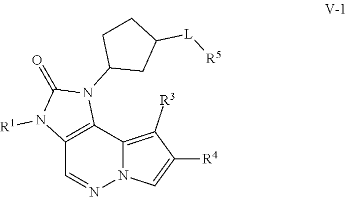

or a pharmaceutically acceptable salt thereof, wherein R¹, L, R⁵, R³, and R⁴ are as defined in classes and subclasses herein, both singly and in combination.

In some embodiments, the present disclosure provides a compound represented by formula V-2:

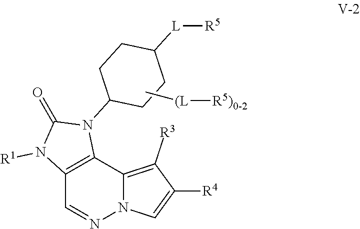

or a pharmaceutically acceptable salt thereof, wherein R¹, L, R⁵, R³, and R⁴ are as defined in classes and subclasses herein, both singly and in combination.

In some embodiments, the present disclosure provides a compound represented by formula VI-1:

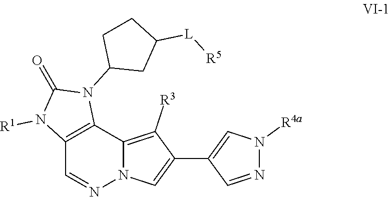

or a pharmaceutically acceptable salt thereof, wherein R¹, L, R⁵, R³, and R^{4a} are as defined in classes and subclasses herein, both singly and in combination.

In some embodiments, the present disclosure provides a compound represented by formula VI-2:

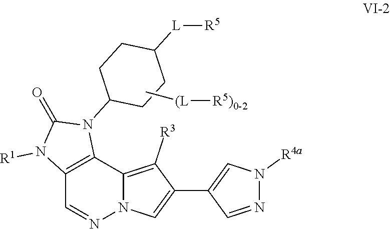

or a pharmaceutically acceptable salt thereof, wherein R¹, L, R⁵, R³, and R^{4a} are as defined in classes and subclasses herein, both singly and in combination.

In some embodiments, the present disclosure provides a compound represented by formula VII-1 or VII-2:

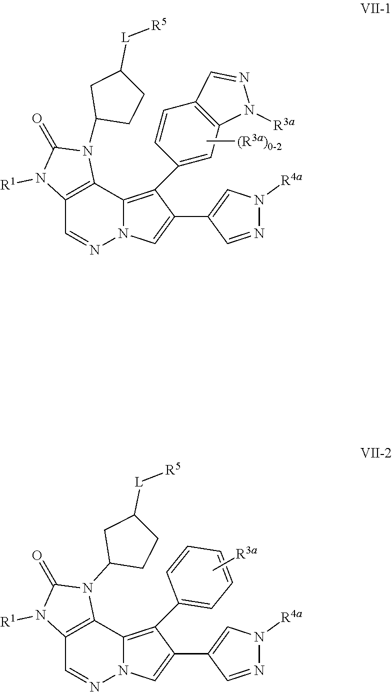

or a pharmaceutically acceptable salt thereof, wherein R¹, L, R⁵, R^{3a}, and R^{4a} are as defined in classes and subclasses herein, both singly and in combination.

In some embodiments, the present disclosure provides a compound represented by formula VII-1:

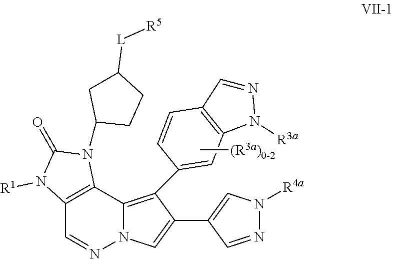

or a pharmaceutically acceptable salt thereof, wherein R¹, L, R⁵, R^{3a}, and R^{4a} are as defined in classes and subclasses herein, both singly and in combination.

In some embodiments, the present disclosure provides a compound represented by formula VII-2:

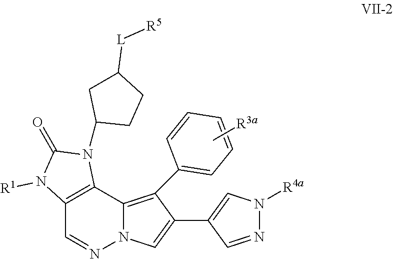

or a pharmaceutically acceptable salt thereof, wherein R¹, L, R⁵, R^{3a}, and R^{4a} are as defined in classes and subclasses herein, both singly and in combination.

In some embodiments, the present disclosure provides a compound represented by formula VII-3:

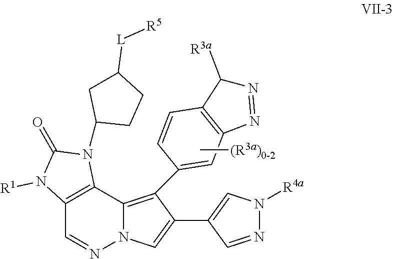

or a pharmaceutically acceptable salt thereof, wherein R¹, L, R⁵, R^{3a}, and R^{4a} are as defined in classes and subclasses herein, both singly and in combination.

In some embodiments, the present disclosure provides a compound represented by formula VII-4:

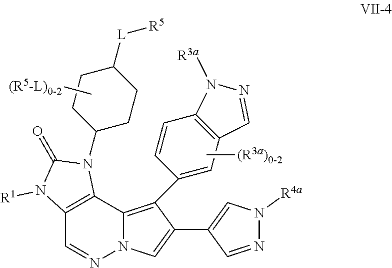

or a pharmaceutically acceptable salt thereof, wherein R¹, L, R⁵, R^{3a}, and R^{4a} are as defined in classes and subclasses herein, both singly and in combination.

In some embodiments, the present disclosure provides a compound represented by formula VII-5:

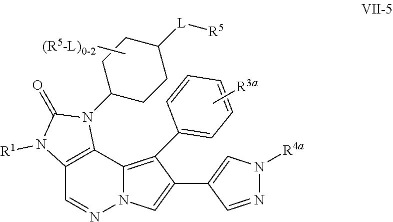

or a pharmaceutically acceptable salt thereof, wherein R¹, L, R⁵, R^{3a}, and R^{4a} are as defined in classes and subclasses herein, both singly and in combination.

In some embodiments, the present disclosure provides a compound represented by formula VII-1, VII-2, VII-3, VII-4, or VII-5, or a pharmaceutically acceptable salt thereof.

In some embodiments, the present disclosure provides a compound represented by formula VIII:

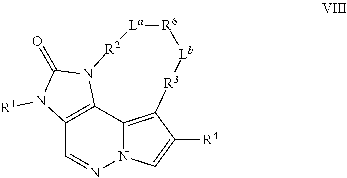

or a pharmaceutically acceptable salt thereof, wherein R¹, R², L^{a}, R⁶, L^{b}, R³, and R⁴ are as defined in classes and subclasses herein, both singly and in combination.

In some embodiments, the present disclosure provides a method of inhibiting JAK2 in a subject, comprising administering to the subject a compound described herein (e.g., a compound of any one of Formula I to VIII) or a pharmaceutical composition comprising a compound described herein.

In some embodiments, the present disclosure provides a method of treating a disease, disorder, or condition associated with JAK2, comprising administering to the subject a compound described herein (e.g., a compound of any one of Formula I to VIII) or a pharmaceutical composition comprising a compound described herein.

In some embodiments, the present disclosure provides a method of treating cancer, comprising administering to a subject in need thereof a compound described herein (e.g., a compound of any one of Formula I to VIII) or a pharmaceutical composition comprising a compound described herein.

In some embodiments, the present disclosure provides a method of treating a hematological malignancy, comprising administering to a subject in need thereof a compound described herein (e.g., a compound of any one of Formula I to VIII) or a pharmaceutical composition comprising a compound described herein.

In some embodiments, the present disclosure provides a method of treating a myeloproliferative neoplasm, comprising administering to a subject in need thereof a compound described herein (e.g., a compound of any one of Formula I to VIII) or a pharmaceutical composition comprising a compound described herein.

### DETAILED DESCRIPTION OF CERTAIN EMBODIMENTS

The present disclosure provides, among other things, compounds useful for inhibiting JAK2. In some embodiments, provided compounds are useful for, among other things, treating and/or preventing diseases, disorders, or conditions associated with JAK2. In some embodiments, the present disclosure encompasses the insight that compounds that bind to the JH2 domain of JAK proteins are particularly useful as inhibitors of JAK2. Such compounds are useful for treating diseases, disorders, and conditions associated with mutations of the JH2 domain that may lead to hyperactivity. For example, the V617F mutation is a mutation that leads to hyperactivity of JAK2. In some embodiments, the discovery of new compounds described herein provides inhibitors that are useful as therapy for diseases and disorders associated with, for example, a V617F mutation to the JH2 domain of JAK2.

In some embodiments, the present disclosure provides a compound represented by Formula I:

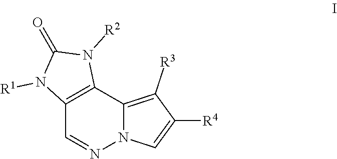

or a pharmaceutically acceptable salt thereof, wherein R¹, R², R³, and R⁴ are as defined in classes and subclasses herein, both singly and in combination.

### Compounds and Definitions

Compounds of this disclosure include those described generally above and are further illustrated by the classes, subclasses, and species disclosed herein. As used herein, the following definitions shall apply unless otherwise indicated. For purposes of this disclosure, the chemical elements are identified in accordance with the Periodic Table of Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomic, and geometric (or conformational) mixtures of provided compounds are within the scope of the disclosure. For example, in some case, Tables 1A-1C show one or more stereoisomers of a compound, and unless otherwise indicated, represents each stereoisomer alone and/or as a mixture. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by ¹³C- or ¹⁴C-enriched carbon are within the scope of this disclosure.

About or approximately: As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within (i.e., ±) 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to the administration of a composition to a subject to achieve delivery of an agent that is, or is included in, a composition to a target site or a site to be treated. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may be parenteral. In some embodiments, administration may be oral. In some particular embodiments, administration may be intravenous. In some particular embodiments, administration may be subcutaneous. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may comprise a prime-and-boost protocol. A prime-and-boost protocol can include administration of a first dose of a pharmaceutical composition (e.g., an immunogenic composition, e.g., a vaccine) followed by, after an interval of time, administration of a second or subsequent dose of a pharmaceutical composition (e.g., an immunogenic composition, e.g., a vaccine). In the case of an immunogenic composition, a prime-and-boost protocol can result in an increased immune response in a patient.

Aliphatic: The term “aliphatic” refers to a straight-chain (i.e., unbranched) or branched, optionally substituted hydrocarbon chain that is completely saturated or that contains one or more units of unsaturation, or a monocyclic or bicyclic hydrocarbon that is completely saturated or that contains one or more units of unsaturation but which is not aromatic (also referred to herein as “carbocyclic” or “cycloaliphatic”), that has a single point of attachment or more than one point of attachment to the rest of the molecule. Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). Suitable aliphatic groups include, but are not limited to, linear or branched, substituted or unsubstituted alkyl, alkenyl, alkynyl groups and hybrids thereof. In some embodiments, “aliphatic” refers to a straight-chain (i.e., unbranched) or branched, optionally substituted hydrocarbon chain that is completely saturated or that contains one or more units of unsaturation that has a single point of attachment or more than one point of attachment to the rest of the molecule (e.g., is multivalent).

Alkyl: The term “alkyl”, used alone or as part of a larger moiety, refers to a saturated, optionally substituted straight or branched hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Alkylene: The term “alkylene” is refers to a bivalent alkyl group. In some embodiments, “alkylene” is a bivalent straight or branched alkyl group. In some embodiments, an “alkylene chain” is a polymethylene group, i.e., —(CH₂)ₙ—, wherein n is a positive integer, e.g., from 1 to 6, from 1 to 4, from 1 to 3, from 1 to 2, or from 2 to 3. An optionally substituted alkylene chain is a polymethylene group in which one or more methylene hydrogen atoms is optionally replaced with a substituent. Suitable substituents include those described below for a substituted aliphatic group and also include those described in the specification herein. It will be appreciated that two substituents of the alkylene group may be taken together to form a ring system. In certain embodiments, two substituents can be taken together to form a 3- to 7-membered ring. The substituents can be on the same or different atoms. The suffix “-ene” or “-enyl” when appended to certain groups herein are intended to refer to a bifunctional moiety of said group. For example, “-ene” or “-enyl”, when appended to “cyclopropyl” becomes “cyclopropylene” or “cyclopropylenyl” and is intended to refer to a bifunctional cyclopropyl group, e.g.,

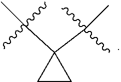 [SMILES: *C1(*)CC1]

Alkenyl: The term “alkenyl”, used alone or as part of a larger moiety, refers to an optionally substituted straight or branched chain or cyclic hydrocarbon group having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Aryl: The term “aryl” refers to monocyclic and bicyclic ring systems having a total of six to fourteen ring members (e.g., C₆-C₁₄), wherein at least one ring in the system is aromatic and wherein each ring in the system contains three to seven ring members. In some embodiments, an “aryl” group contains between six and twelve total ring members (e.g., C₆-C₁₂). The term “aryl” may be used interchangeably with the term “aryl ring”. In certain embodiments, “aryl” refers to an aromatic ring system which includes, but not limited to, phenyl, biphenyl, naphthyl, anthracyl and the like, which may bear one or more substituents. Unless otherwise specified, “aryl” groups are hydrocarbons. In some embodiments, an “aryl” ring system is an aromatic ring (e.g., phenyl) that is fused to a non-aromatic ring (e.g., cycloalkyl). Examples of aryl rings include that are fused include

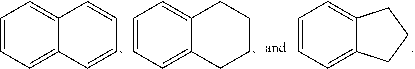

Bicyclic: The term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

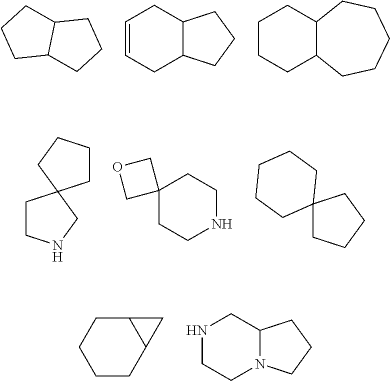

Exemplary bridged bicyclics include:

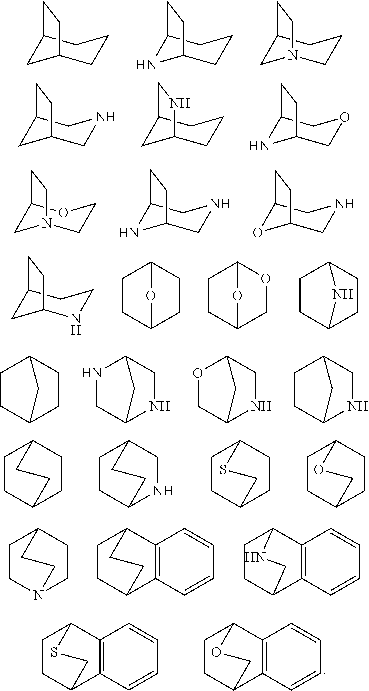

Biological sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Cycloaliphatic: As used herein, the term “cycloaliphatic” refers to a monocyclic C₃₋₈ hydrocarbon or a bicyclic C₆₋₁₂ hydrocarbon that is completely saturated or that contains one or more units of unsaturation, but which is not aromatic, that has a single point or more than one points of attachment to the rest of the molecule.

Cycloalkyl: As used herein, the term “cycloalkyl” refers to an optionally substituted saturated ring monocyclic or polycyclic system of about 3 to about 10 ring carbon atoms. Exemplary monocyclic cycloalkyl rings include cyclopropyl, cyclobutyl, cyclopentyl, cyclohexyl, and cycloheptyl.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

Effective amount: The term “effective amount” refers to the amount of a compound sufficient to effect beneficial or desired results (e.g., a therapeutic, ameliorative, inhibitory, or preventative result). An effective amount can be administered in one or more administrations, applications, or dosages and is not intended to be limited to a particular formulation or administration route.

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Halogen: The term “halogen” or “halo” means F, Cl, Br, or I.

Heteroaliphatic: The term “heteroaliphatic” or “heteroaliphatic group”, as used herein, denotes an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. The term “heteroatom” refers to nitrogen, oxygen, or sulfur, and includes any oxidized form of nitrogen or sulfur, and any quaternized form of a basic nitrogen. The term “nitrogen” also includes a substituted nitrogen. Unless otherwise specified, heteroaliphatic groups contain 1-10 carbon atoms wherein 1-3 carbon atoms are optionally and independently replaced with heteroatoms selected from oxygen, nitrogen, and sulfur. In some embodiments, heteroaliphatic groups contain 1-4 carbon atoms, wherein 1-2 carbon atoms are optionally and independently replaced with heteroatoms selected from oxygen, nitrogen, and sulfur. In yet other embodiments, heteroaliphatic groups contain 1-3 carbon atoms, wherein 1 carbon atom is optionally and independently replaced with a heteroatom selected from oxygen, nitrogen, and sulfur. Suitable heteroaliphatic groups include, but are not limited to, linear or branched, heteroalkyl, heteroalkenyl, and heteroalkynyl groups. For example, a 1- to 10 atom heteroaliphatic group includes the following exemplary groups: —O—CH₃, —CH₂—O—CH₃, —O—CH₂—CH₂—O—CH₂—CH₂—O—CH₃, and the like.

Heteroaryl: The terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 π electrons shared in a cyclic array; and having, in addition to carbon atoms, from one to five heteroatoms. Exemplary heteroaryl groups include, without limitation, thienyl, furanyl, pyrrolyl, imidazolyl, pyrazolyl, triazolyl, tetrazolyl, oxazolyl, oxadiazolyl, thiazolyl, isothiazolyl, thiadiazolyl, pyridyl, pyridonyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridinyl, thienopyrimidinyl, triazolopyridinyl, and benzoisoxazolyl. The terms “heteroaryl” and “heteroar-”, as used herein, also include groups in which a heteroaromatic ring is fused to one or more aryl, cycloaliphatic, or heterocyclyl rings (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, and benzoisoxazolyl. The term “heteroaryl” may be used interchangeably with the terms “heteroaryl ring”, “heteroaryl group”, or “heteroaromatic”, any of which terms include rings that are optionally substituted.

Heteroatom: The term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, and “heterocyclic ring” are used interchangeably and refer to a stable 3- to 8-membered monocyclic or 4- to 10-membered bicyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. A bicyclic heterocyclic ring also includes groups in which the heterocyclic ring is fused to one or more aryl, heteroaryl, or cycloaliphatic rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 6- to 11-membered spirocyclic fused heterocyclic ring having, in addition to carbon atoms, one or more heteroatoms as defined above (e.g., one, two, three or four heteroatoms)). A bicyclic heterocyclic ring can also be a bridged ring system (e.g., 7- to 11-membered bridged heterocyclic ring having one, two, or three bridging atoms.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intra-arterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Partially Unsaturated: As used herein, the term “partially unsaturated”, when referring to a ring moiety, means a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, the active agent is present in unit dose amount appropriate for administration in a therapeutic or dosing regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and/or lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences 1977, 66(1), 1-19; P. Gould, International J. of Pharmaceutics 1986, 33, 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Substituted or optionally substituted: As described herein, compounds of this disclosure may contain “optionally substituted” moieties. In general, the term “substituted,” whether preceded by the term “optionally” or not, means that one or more hydrogens of the designated moiety are replaced with a suitable substituent (i.e., as described below for optionally substituted groups). “Substituted” applies to one or more hydrogens that are either explicit or implicit from the structure (e.g.,

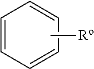

refers to at least

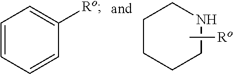

refers to at least

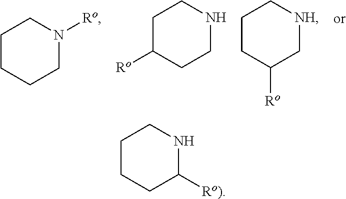

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)(NH)R^{∘}; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁-6 aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂, —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OsiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR^{●}₂, ═NNHC(O)R^{●}, ═NNHC(O)OR^{●}, =NNHS(O)₂R^{●}, =NR^{●}, =NOR^{●}, —O(C(R^{●}₂))₂₋₃O—, or —S(C(R^{●}₂))₂₋₃S—, wherein each independent occurrence of R^{●} is selected from hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{●}₂)₂₋₃O—, wherein each independent occurrence of R^{●} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer.

In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid.

In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent.

Those of ordinary skill in the art, reading the present disclosure, will appreciate that certain small molecule compounds described herein may be provided and/or utilized in any of a variety of forms such as, for example, crystal forms (e.g., polymorphs, solvates, etc), salt forms, protected forms, pro-drug forms, ester forms, isomeric forms (e.g., optical and/or structural isomers), isotopic forms, etc.

Those of ordinary skill in the art will appreciate that certain small molecule compounds have structures that can exist in one or more stereoisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form.

Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms.

Those of skill in the art will appreciate that certain small molecule compounds have structures that permit isotopic substitution (e.g., ²H or ³H for H; ¹¹C, ¹³C or ¹⁴C for ¹²C; ¹³N or ⁵N for ¹⁴N; ¹⁷O or ¹⁸O for ¹⁶O; ³⁶Cl for ¹⁵Cl or ³⁷Cl; ¹⁸F for ¹⁹F; ¹³¹I for ¹²⁷I; etc.). In some embodiments, such a small molecule may be utilized in accordance with the present disclosure in one or more isotopically modified forms, or mixtures thereof.

In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form.

In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Those skilled in the art will further appreciate that, in small molecule structures, the symbol [symbol] , as used herein, refers to a point of attachment between two atoms. Additionally or alternatively, the symbol [symbol] refers to a point of attachment ring in a spirocyclic manner. Those skilled in the art will further appreciate, in small molecule structures, the symbol [symbol] , as used herein, represents a single or double bond between two atoms in a ring structure. For example:

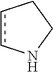

is intended to encompass both:

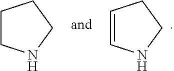 [SMILES: C1CCNC1 ; "and" ; C1=CNCC1 ; "."]

Treat: As used herein, the term “treat” (also “treatment” or “treating”) refers to any administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition.

As used herein, a “floating” substituent drawn on a ring indicates that a substituent may exist at any position of the ring. For example, for a ring such as

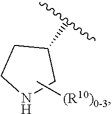

a R¹⁰ substituent may exist at any position and may replace a hydrogen atom of any substitutable carbon or nitrogen atom. In some embodiments, a “floating” substituent drawn on one ring of a polycyclic moiety, indicates that a substituent may exist on any ring of the polycyclic moiety (e.g.,

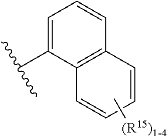

is equivalent to

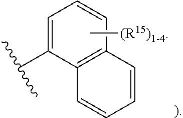

### Provided Compounds

In some embodiments, the present disclosure provides a compound of Formula I:

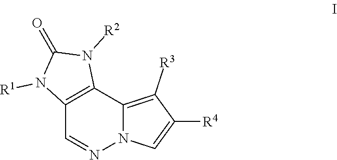

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is optionally substituted with one or more instances of -L-R⁵ or R⁷;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a}), —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
each R⁷ is independently selected from the group consisting of halogen, —OH, —SH, and —NH₂;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a}; or
R² and R³ are connected by *-L^{a}-R⁶-L^{b}-**, wherein * represents a point of attachment to R², and ** represents a point of attachment to R³;
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
L^{b} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
R⁶ is optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, or optionally substituted C₆-C₁₀ aryl;
R⁴ is C₁-C₆ aliphatic, —CN, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)R^{a}, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, the present disclosure provides a compound of Formula I, or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is optionally substituted with one or more instances of -L-R⁵ or R⁷; each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a}), —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
each R⁷ is independently selected from the group consisting of halogen, —OH, —SH, and —NH₂;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a}; or
R² and R³ are connected by *-L^{a}-R⁶-L^{b}-**, wherein * represents a point of attachment to R², and ** represents a point of attachment to R³;
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
L^{b} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
R⁶ is optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, or optionally substituted C₆-C₁₀ aryl;
R⁴ is C₁-C₆ aliphatic, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, the present disclosure provides a compound of Formula I, or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is optionally substituted with one or more instances of -L-R⁵;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a}), —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a}; or
R² and R³ are connected by *-L^{a}-R⁶-L^{b}-**, where * represents a point of attachment to R², and ** represents a point of attachment to R³;
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
L^{b} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
R⁶ is optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, or optionally substituted C₆-C₁₀ aryl;
R⁴ is C₁-C₆ aliphatic, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, a compound of Formula I is a compound represented by Formula II-1 or II-2:

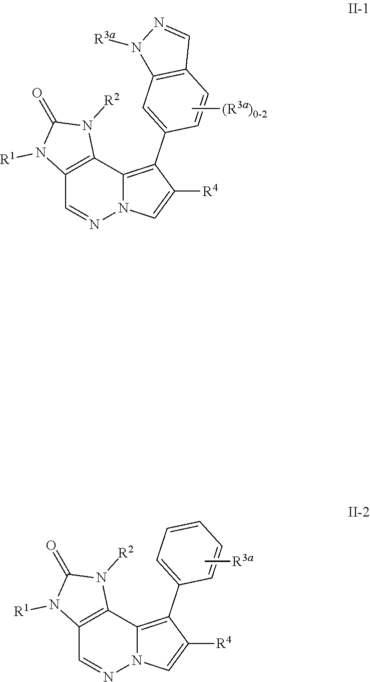

or a pharmaceutically acceptable salt thereof, wherein R¹, R², R^{3a}, and R⁴ are as defined in classes and subclasses herein, both singly and in combination.

In some embodiments, the present disclosure provides a compound represented by formula II-3:

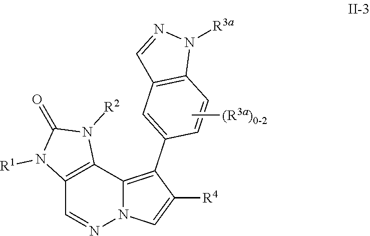

or a pharmaceutically acceptable salt thereof, wherein R¹, R², R^{3a}, and R⁴ are as defined in classes and subclasses herein, both singly and in combination.

In some embodiments, a compound of Formula I is a compound represented by Formula III:

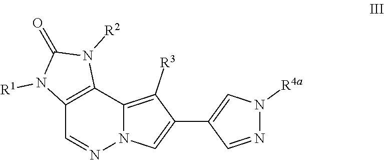

or a pharmaceutically acceptable salt thereof, wherein R¹, R², R³, and R^{4a} are as defined in classes and subclasses herein, both singly and in combination.

In some embodiments, a compound of Formula I is a compound represented by Formula IV-1 or IV-2:

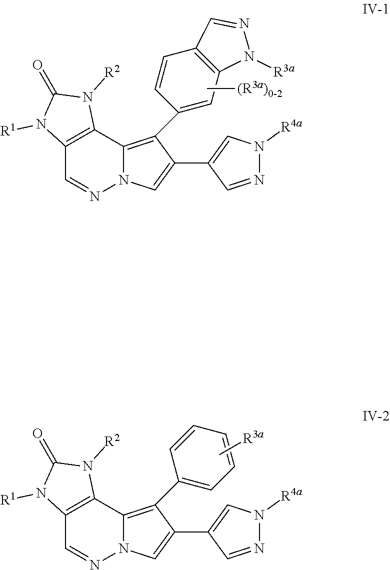

or a pharmaceutically acceptable salt thereof, wherein R¹, R², R^{3a}, and R^{4a} are as defined in classes and subclasses herein, both singly and in combination.

In some embodiments, the present disclosure provides a compound represented by formula IV-3:

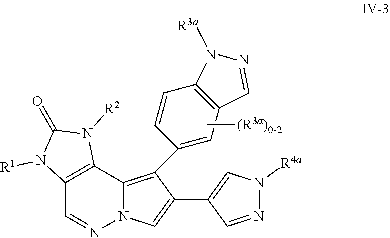

or a pharmaceutically acceptable salt thereof, wherein R¹, R², R^{3a}, and R^{4a} are as defined in classes and subclasses herein, both singly and in combination.

In some embodiments, a compound of Formula I is a compound represented by Formula V-1:

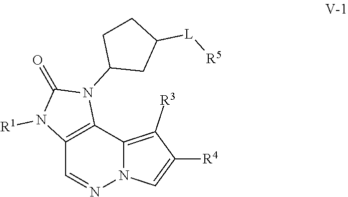

or a pharmaceutically acceptable salt thereof, wherein R¹, L, R⁵, R³, and R⁴ are as defined in classes and subclasses herein, both singly and in combination.

In some embodiments, the present disclosure provides a compound represented by formula V-2:

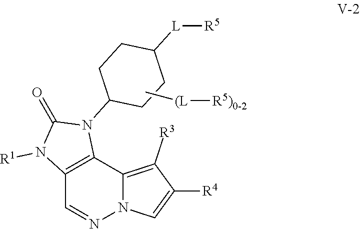

or a pharmaceutically acceptable salt thereof, wherein R¹, L, R⁵, R³, and R⁴ are as defined in classes and subclasses herein, both singly and in combination.

In some embodiments, the present disclosure provides a compound represented by formula VI:

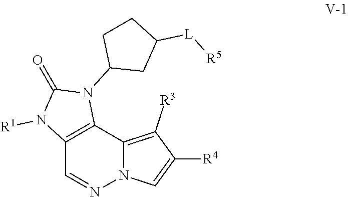

or a pharmaceutically acceptable salt thereof, wherein R¹, L, R⁵, R³, and R^{4a} are as defined in classes and subclasses herein, both singly and in combination.

In some embodiments, the present disclosure provides a compound represented by formula VII-1 or VII-2:

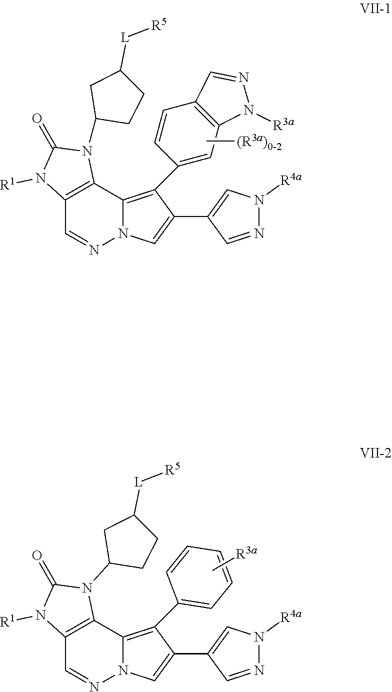

or a pharmaceutically acceptable salt thereof, wherein R¹, R², R^{3a}, and R^{4a} are as defined in classes and subclasses herein, both singly and in combination.

In some embodiments, the present disclosure provides a compound represented by formula VII-3:

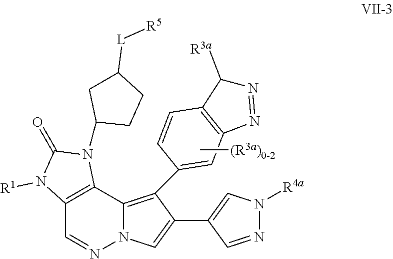

or a pharmaceutically acceptable salt thereof, wherein R¹, L, R⁵, R^{3a}, and R^{4a} are as defined in classes and subclasses herein, both singly and in combination.

In some embodiments, the present disclosure provides a compound represented by formula VII-4 or VII-5:

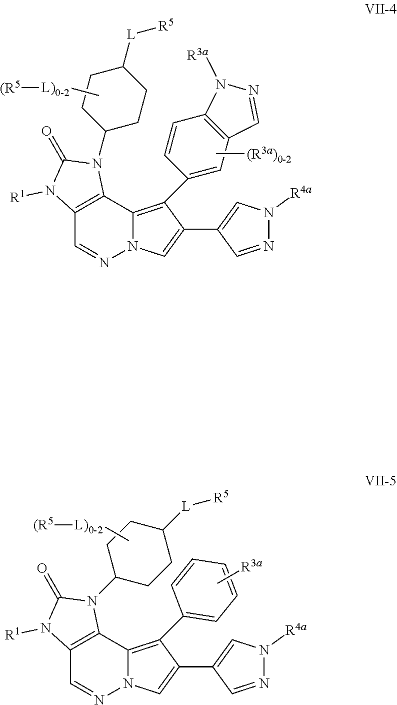

or a pharmaceutically acceptable salt thereof, wherein R¹, L, R⁵, R^{3a}, and R^{4a} are as defined in classes and subclasses herein, both singly and in combination.

In some embodiments, the present disclosure provides a compound represented by formula VIII:

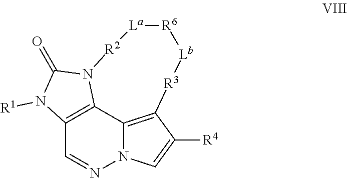

or a pharmaceutically acceptable salt thereof, wherein R¹, R², L^{a}, R⁶, L^{b}, R³, and R⁴ are as defined in classes and subclasses herein, both singly and in combination.

In some embodiments, the present disclosure provides a compound represented by formula IX:

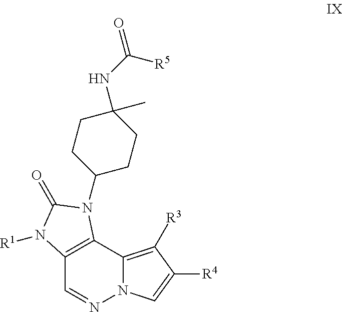

or a pharmaceutically acceptable salt thereof, wherein R⁵ is optionally substituted cyclopropyl; and R¹, R³, and R⁴ are as defined in classes and subclasses herein, both singly and in combination. In some embodiments, R⁵ is cyclopropyl optionally substituted with one or more halogen, —(CH₂)₀₋₄R^{∘}, or —CN. In some embodiments, R⁵ is cyclopropyl optionally substituted with one or more fluoro, a 3- to 6-membered aryl ring having 1-4 heteroatoms independently selected from nitrogen, oxygen, or sulfur optionally substituted with C₁₋₄ aliphatic, or —CN. In some embodiments, R⁵ is cyclopropyl optionally substituted with one or more halogen (e.g., fluoro). In some embodiments, R⁵ is

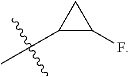 [SMILES: *C1CC1F]

As described herein, with respect to any formula provided herein (e.g., any of Formulae I-IX), R¹ is hydrogen or optionally substituted C₁-C₆ aliphatic. In some embodiments, R¹ is hydrogen. In some embodiments, R¹ is optionally substituted C₁-C₆ aliphatic. In some embodiments, R¹ is optionally substituted C₁-C₃ aliphatic. In some embodiments, R¹ is C₁-C₆ aliphatic. In some embodiments, R¹ is methyl (—CH₃) or deuterated methyl (e.g., one or more H atoms of methyl have been replaced with deuterium, i.e., —CH₂D, —CHD₂, or -CD₃). In some embodiments, R¹ is —CH₃ or -CD₃. In some embodiments, R¹ is —CH₃. In some embodiments, R¹ is —CD₃.

As described herein, with respect to any formula provided herein (e.g., any of Formulae I-IX), R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is optionally substituted with one or more instances of -L-R⁵ or R⁷. In some embodiments, R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is substituted with one or more instances of -L-R⁵ or R⁷. In some embodiments, R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is substituted with 1-2 instances of -L-R⁵ and with 0-2 instances of R⁷. In some embodiments, R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is substituted with 0-2 instances of -L-R⁵ and with 1-2 instances of R⁷.

In some embodiments, R² is C₁-C₆ aliphatic, C₃-C₁₂ cycloaliphatic, or 4- to 10-membered heterocycle comprising 1 to 4 heteroatoms selected from N, O, and S, wherein R² is optionally substituted with one or more instances of -L-R⁵ or R⁷. In some embodiments, R² is C₁-C₆ aliphatic, C₃-C₁₂ cycloaliphatic, or 4- to 10-membered heterocycle comprising 1 to 4 heteroatoms selected from N, O, and S, wherein R² is substituted with 0-2 instances of -L-R⁵ or R⁷. In some embodiments, R² is C₁-C₆ aliphatic, C₃-C₁₂ cycloaliphatic, or 4- to 10-membered heterocycle comprising 1 to 4 heteroatoms selected from N, O, and S, wherein R² is optionally substituted with one instance of -L-R⁵ or R⁷. In some embodiments, R² is C₃-C₁₂ cycloaliphatic or 4- to 10-membered heterocycle comprising 1 to 4 heteroatoms selected from N, O, and S; and wherein R² is substituted with 0-2 instances of -L-R⁵ or R⁷. In some embodiments, R² is C₃-C₁₂ cycloaliphatic or 4- to 10-membered heterocycle comprising 1 to 4 heteroatoms selected from N, O, and S; and wherein R² is substituted with 1-2 instances of -L-R⁵ or R⁷. In some embodiments, R² is C₃-C₁₂ cycloaliphatic or 4- to 10-membered heterocycle comprising 1 to 4 heteroatoms selected from N, O, and S; and wherein R² is substituted with one instance of -L-R⁵ or R⁷. In some embodiments, R² is C₃-C₇ monocyclic cycloaliphatic or 4- to 6-membered monocyclic heterocycle comprising 1 to 2 heteroatoms selected from N, O, and S; and wherein R² is substituted with 0-2 instances of -L-R⁵ or R⁷. In some embodiments, R² is C₃-C₇ monocyclic cycloaliphatic or 4- to 6-membered monocyclic heterocycle comprising 1 to 2 heteroatoms selected from N, O, and S; and wherein R² is substituted with 1-2 instances of -L-R⁵ or R⁷. In some embodiments, R² is C₃-C₇ monocyclic cycloaliphatic or 4- to 6-membered monocyclic heterocycle comprising 1 to 2 heteroatoms selected from N, O, and S; and wherein R² is substituted with one instance of -L-R⁵ or R⁷.

In some embodiments, R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is optionally substituted with one or more instances of -L-R⁵. In some embodiments, R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is substituted with one or more instances of -L-R⁵. In some embodiments, R² is C₁-C₆ aliphatic, C₃-C₁₂ cycloaliphatic, or 4- to 10-membered heterocycle comprising 1 to 4 heteroatoms selected from N, O, and S, wherein R² is optionally substituted with one or more instances of -L-R⁵. In some embodiments, R² is C₁-C₆ aliphatic, C₃-C₁₂ cycloaliphatic, or 4- to 10-membered heterocycle comprising 1 to 4 heteroatoms selected from N, O, and S, wherein R² is substituted with 1-2 instances of -L-R⁵. In some embodiments, R² is C₁-C₆ aliphatic, C₃-C₁₂ cycloaliphatic, or 4- to 10-membered heterocycle comprising 1 to 4 heteroatoms selected from N, O, and S, wherein R² is optionally substituted with one instance of -L-R⁵. In some embodiments, R² is C₃-C₁₂ cycloaliphatic or 4- to 10-membered heterocycle comprising 1 to 4 heteroatoms selected from N, O, and S; and wherein R² is substituted with one instance of -L-R⁵. In some embodiments, R² is C₃-C₇ monocyclic cycloaliphatic or 4- to 6-membered monocyclic heterocycle comprising 1 to 4 heteroatoms selected from N, O, and S; and wherein R² is substituted with one instance of -L-R⁵.

In some embodiments, R² is C₁-C₆ aliphatic, and wherein R² is optionally substituted with one or more instances of -L-R⁵ or R⁷. In some embodiments, R² is C₁-C₆ aliphatic, and wherein R² is optionally substituted with one or more instances of -L-R⁵. In some embodiments, R² is C₁-C₆ aliphatic, and wherein R² is optionally substituted with one instance of -L-R⁵. In some embodiments, R² is 2- to 10-membered heteroaliphatic, and wherein R² is optionally substituted with one or more instances of -L-R⁵ or R⁷. In some embodiments, R² is 2- to 10-membered heteroaliphatic, and wherein R² is optionally substituted with one or more instances of -L-R⁵. In some embodiments, R² is 2- to 10-membered heteroaliphatic, and wherein R² is optionally substituted with one instance of -L-R⁵.

In some embodiments, R² is C₃-C₁₂ cycloaliphatic, and wherein R² is optionally substituted with one or more instances of -L-R⁵ or R⁷. In some embodiments, R² is C₃-C₁₂ cycloaliphatic, and wherein R² is substituted with 1-2 instances of -L-R⁵ or R⁷. In some embodiments, R² is C₃-C₆ cycloaliphatic, and wherein R² is optionally substituted with one or more instances of -L-R⁵ or R⁷. In some embodiments, R² is C₃-C₆ cycloaliphatic, and wherein R² is substituted with 1-2 instances of -L-R⁵ or R⁷. In some embodiments, R² is C₄-C₆ cycloaliphatic, and wherein R² is optionally substituted with one or more instances of -L-R⁵ or R⁷. In some embodiments, R² is C₄-C₆ cycloaliphatic, and wherein R² is substituted with 1-2 instances of -L-R⁵ or R⁷. In some embodiments, R² is C₃-C₆ cycloaliphatic selected from cyclopropyl, cyclobutyl, cyclopentyl, and cyclohexyl, and wherein R² is optionally substituted with one or more instances of -L-R⁵ or R⁷. In some embodiments, R² is C₃-C₆ cycloaliphatic selected from cyclopropyl, cyclobutyl, cyclopentyl, and cyclohexyl, and wherein R² is substituted with 1-2 instances of -L-R⁵ or R⁷. In some embodiments, R² is cyclopropyl, and wherein R² is optionally substituted with one or more instances of -L-R⁵ or R⁷. In some embodiments, R² is cyclopropyl, and wherein R² is substituted with 1-2 instances of -L-R⁵ or R⁷. In some embodiments, R² is cyclobutyl, and wherein R² is optionally substituted with one or more instances of -L-R⁵ or R⁷. In some embodiments, R² is cyclobutyl, and wherein R² is substituted with 1-2 instances of -L-R⁵ or R⁷. In some embodiments, R² is cyclopentyl, and wherein R² is optionally substituted with one or more instances of -L-R⁵ or R⁷. In some embodiments, R² is cyclopentyl, and wherein R² is substituted with 1-2 instances of -L-R⁵ or R⁷. In some embodiments, R² is cyclohexyl, and wherein R² is optionally substituted with one or more instances of -L-R⁵ or R⁷. In some embodiments, R² is cyclohexyl, and wherein R² is substituted with 1-2 instances of -L-R⁵ or R⁷.

In some embodiments, R² is C₃-C₁₂ cycloaliphatic, and wherein R² is optionally substituted with one or more instances of -L-R⁵. In some embodiments, R² is C₃-C₁₂ cycloaliphatic, and wherein R² is optionally substituted with one instance of -L-R⁵. In some embodiments, R² is C₃-C₁₂ cycloaliphatic. In some embodiments, R² is C₃-C₆ cycloaliphatic, and wherein R² is optionally substituted with one or more instances of -L-R⁵. In some embodiments, R² is C₃-C₆ cycloaliphatic, and wherein R² is optionally substituted with one instance of -L-R⁵. In some embodiments, R² is C₄-C₆ cycloaliphatic, and wherein R² is optionally substituted with one or more instances of -L-R⁵. In some embodiments, R² is C₃-C₆ cycloaliphatic selected from cyclopropyl, cyclobutyl, cyclopentyl, and cyclohexyl, and wherein R² is optionally substituted with one or more instances of -L-R⁵. In some embodiments, R² is C₃-C₆ cycloaliphatic selected from cyclopropyl, cyclobutyl, and cyclopentyl, and wherein R² is optionally substituted with one or more instances of -L-R⁵. In some embodiments, R² is cyclopropyl, and wherein R² is optionally substituted with one or more instances of -L-R⁵. In some embodiments, R² is cyclobutyl, and wherein R² is optionally substituted with one or more instances of -L-R⁵. In some embodiments, R² is cyclopentyl, and wherein R² is optionally substituted with one or more instances of -L-R⁵. In some embodiments, R² is cyclohexyl, and wherein R² is optionally substituted with one or more instances of -L-R⁵.

In some embodiments, R² is

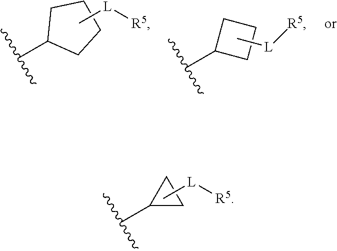

In some embodiments, R² is

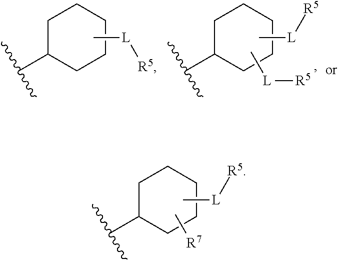

In some embodiments R² is

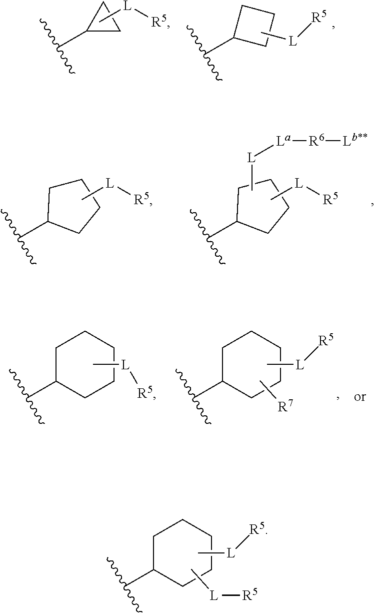

In some embodiments, R² is

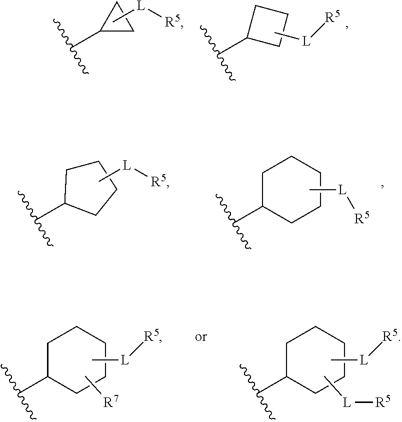

In some embodiments, R² is

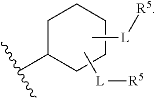

In some embodiments, R² is

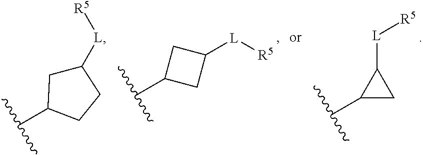

In some embodiments, R² is

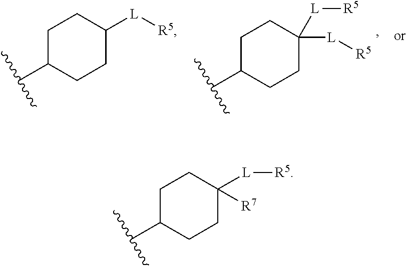

In some embodiments, R² is

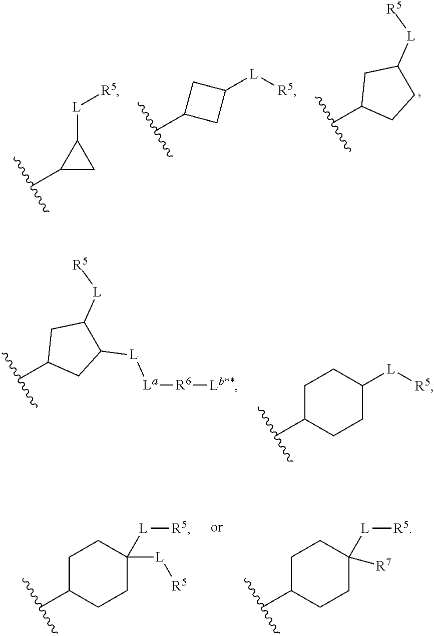

In some embodiments, R² is

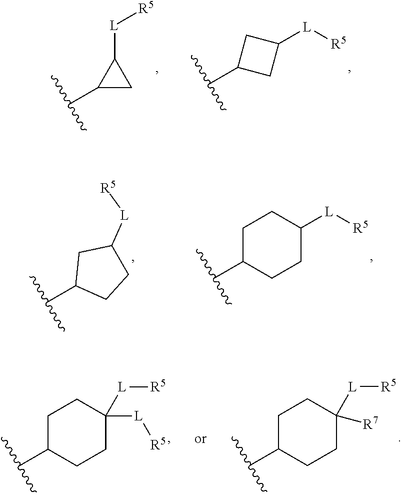

In some embodiments, R² is

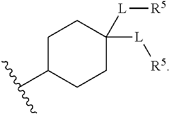

In some embodiments, R² is

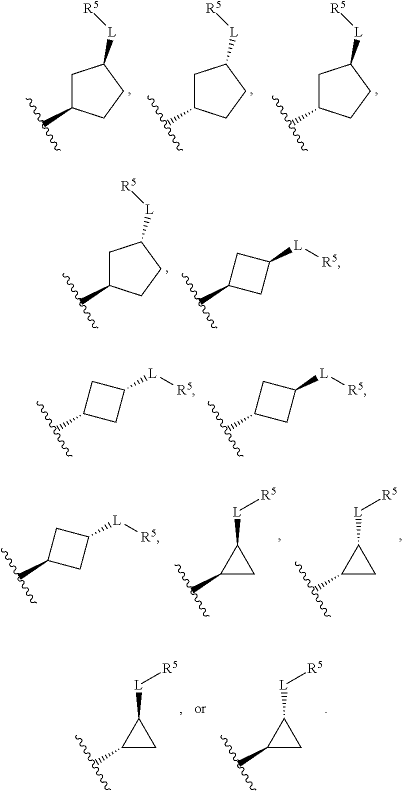

In some embodiments, R² is

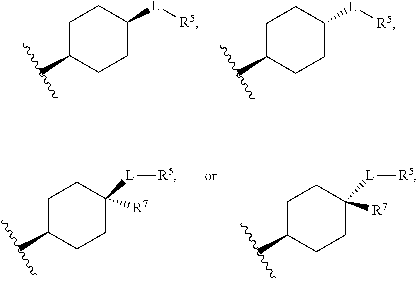

In some embodiments, R² is

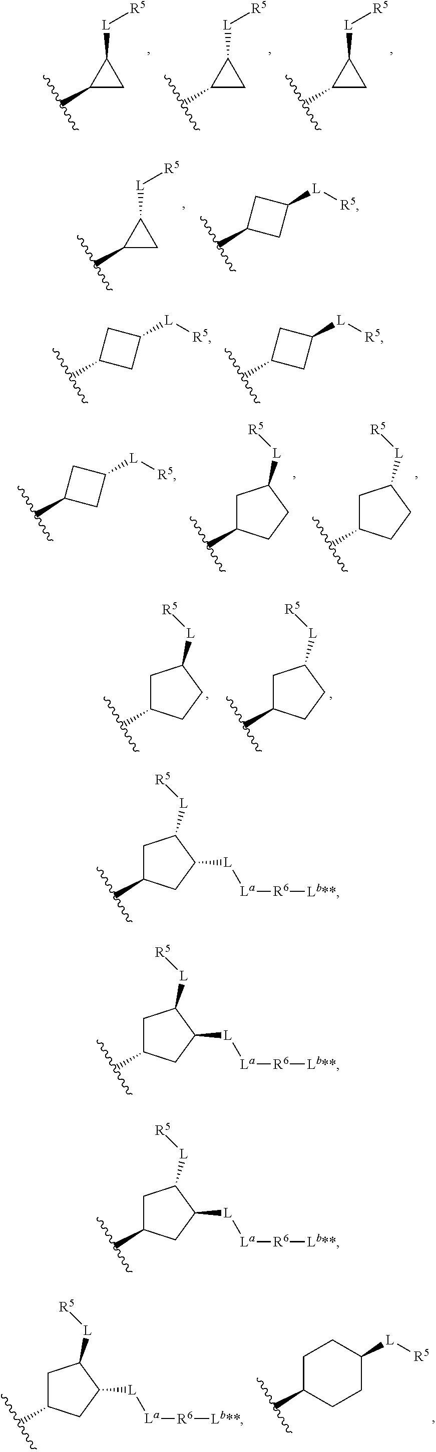

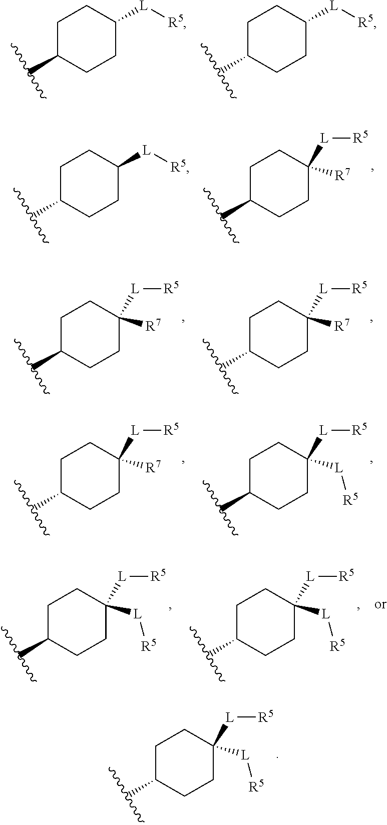

In some embodiments, R² is

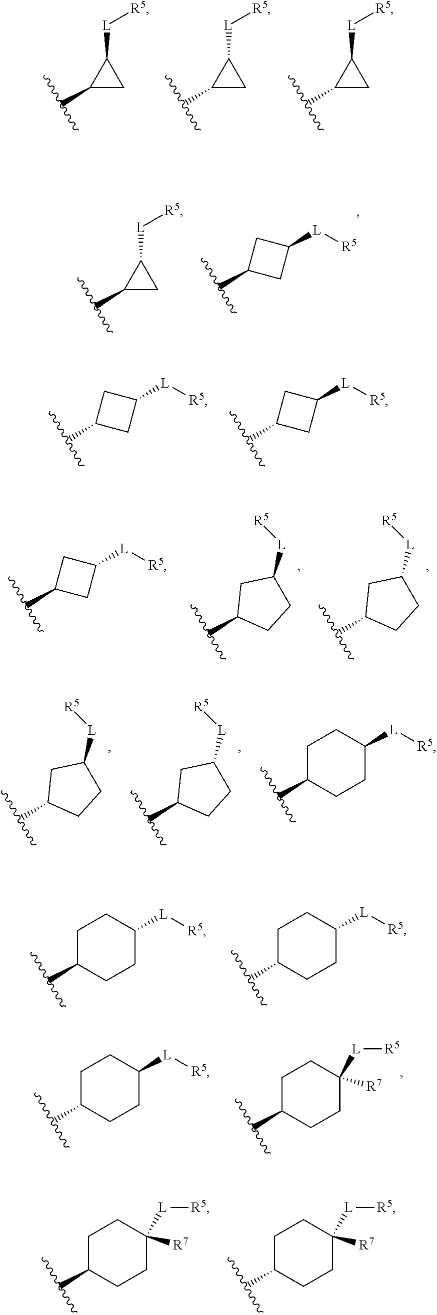

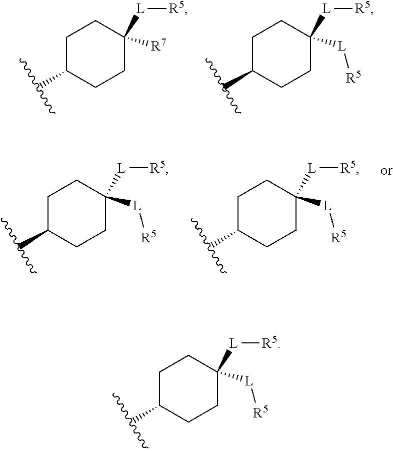

In some embodiments, R² is 4- to 10-membered heterocycle comprising 1 to 4 heteroatoms selected from N, O, and S, and wherein R² is optionally substituted with one or more instances of -L-R⁵ or R⁷. In some embodiments, R² is 4- to 10-membered heterocycle comprising 1 to 4 heteroatoms selected from N, O, and S, and wherein R² is substituted with 1-2 instances of -L-R⁵ or R⁷. In some embodiments, R² is 4- to 6-membered heterocycle comprising 1 to 2 heteroatoms selected from N, O, and S, and wherein R² is optionally substituted with one or more instances of -L-R⁵ or R⁷. In some embodiments, R² is 4- to 6-membered heterocycle comprising 1 to 2 heteroatoms selected from N, O, and S, and wherein R² is substituted with 1-2 instances of -L-R⁵ or R⁷. In some embodiments, R² is oxetane, tetrahydrofuran, or pyran, and wherein R² is optionally substituted with one or more instances of -L-R⁵ or R⁷. In some embodiments, R² is oxetane, tetrahydrofuran, or pyran, and wherein R² is substituted with 1-2 instances of -L-R⁵ or R⁷.

In some embodiments, R² is 4- to 10-membered heterocycle comprising 1 to 4 heteroatoms selected from N, O, and S, and wherein R² is optionally substituted with one or more instances of -L-R⁵. In some embodiments, R² is 4- to 10-membered heterocycle comprising 1 to 4 heteroatoms selected from N, O, and S, and wherein R² is optionally substituted with one instance of -L-R⁵. In some embodiments, R² is 4- to 10-membered heterocycle comprising 1 to 4 heteroatoms selected from N, O, and S. In some embodiments, R² is 4- to 6-membered heterocycle comprising 1 to 4 heteroatoms selected from N, O, and S, and wherein R² is optionally substituted with one or more instances of -L-R⁵. In some embodiments, R² is 4- to 6-membered heterocycle comprising 1 to 4 heteroatoms selected from N, O, and S, and wherein R² is optionally substituted with one instance of -L-R⁵. In some embodiments, R² is 4- to 6-membered heterocycle comprising 1 to 2 heteroatoms selected from N, O, and S, and wherein R² is optionally substituted with one or more instances of -L-R⁵. In some embodiments, R² is oxetane, tetrahydrofuran, or pyran, and wherein R² is optionally substituted with one or more instances of -L-R⁵.

In some embodiments, R² is

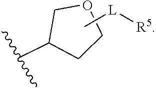

In some embodiments, R² is

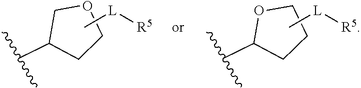

In some embodiments, R² is

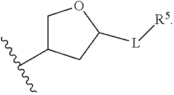

In some embodiments, R² is

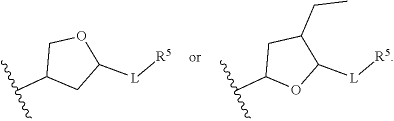

In some embodiments, R² is

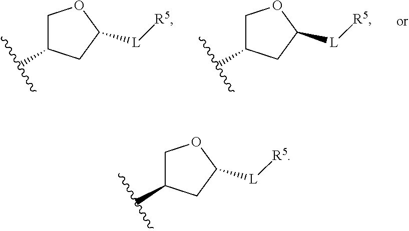

In some embodiments, R² is

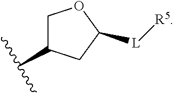

In some embodiments, R² is

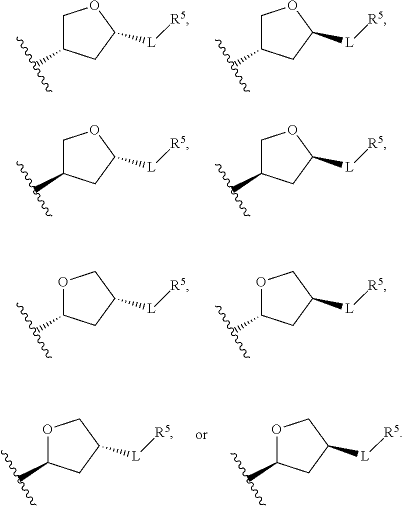

In some embodiments, R² is

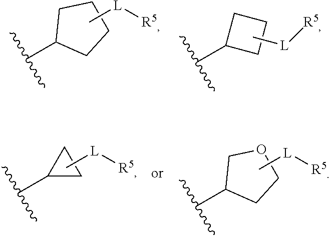

In some embodiments, R² is

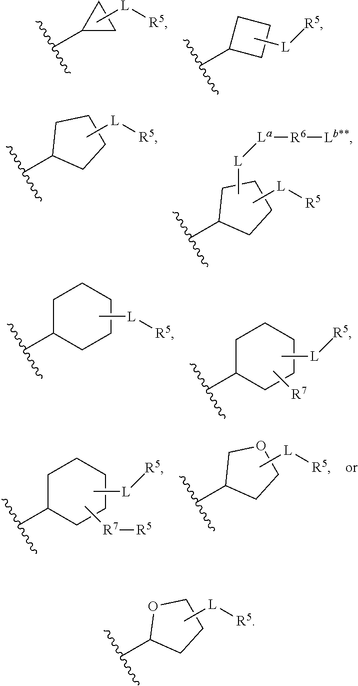

In some embodiments, R² is

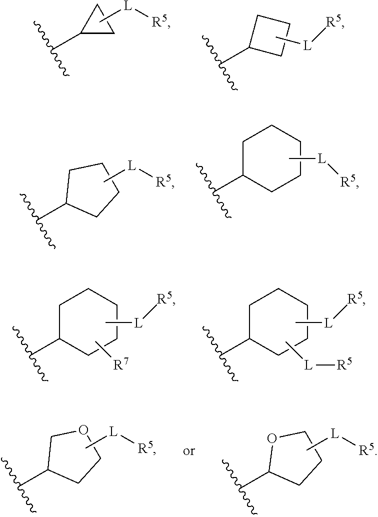

In some embodiments, R² is

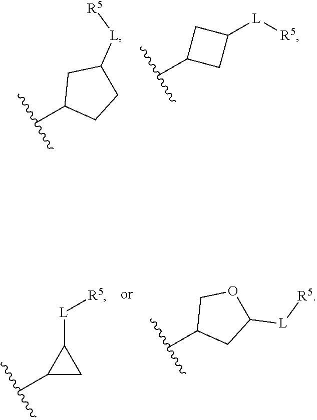

In some embodiments, R² is

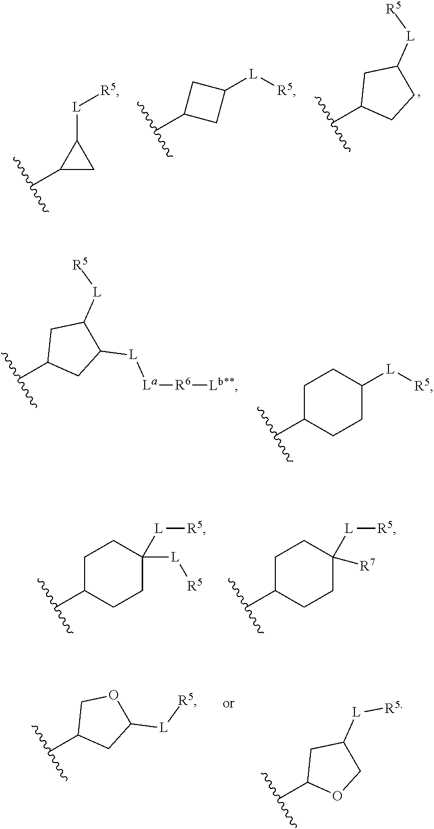

In some embodiments, R² is

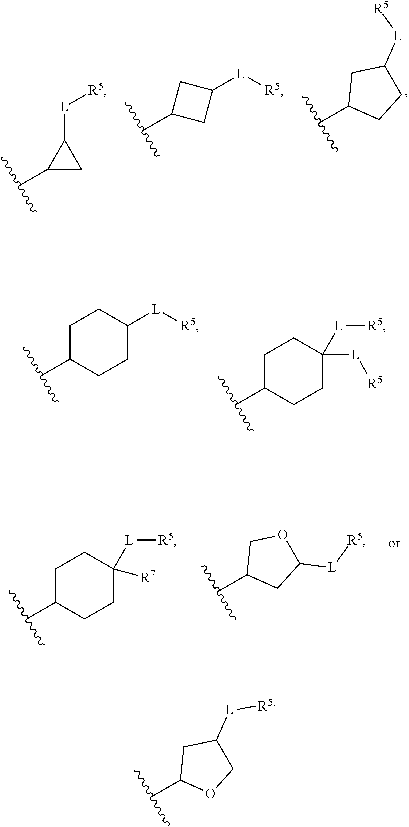

In some embodiments, R² is

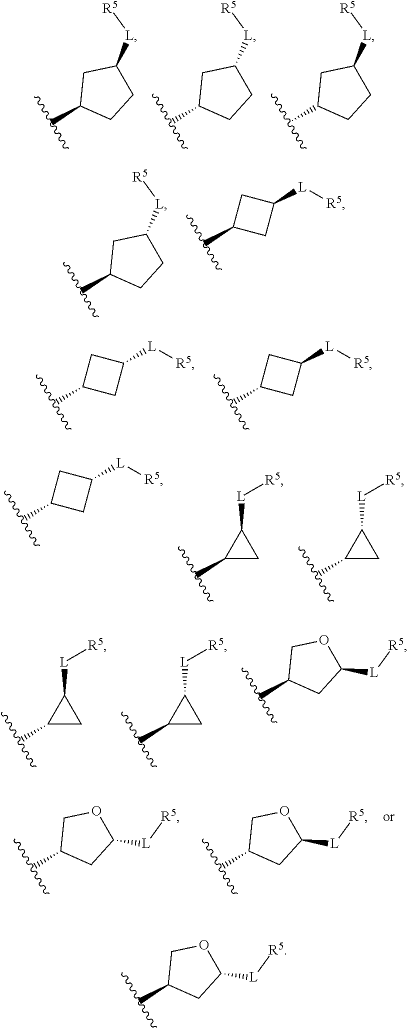

In some embodiments, R² is

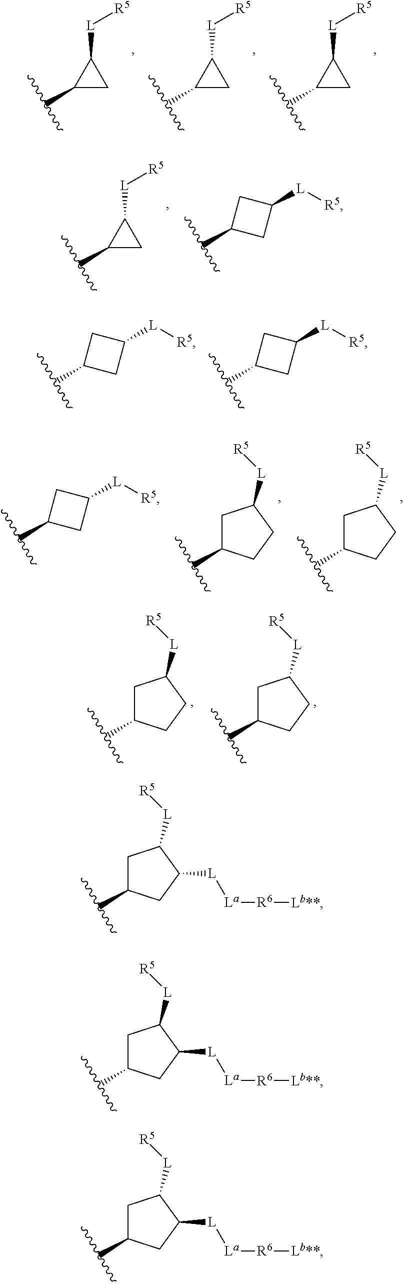

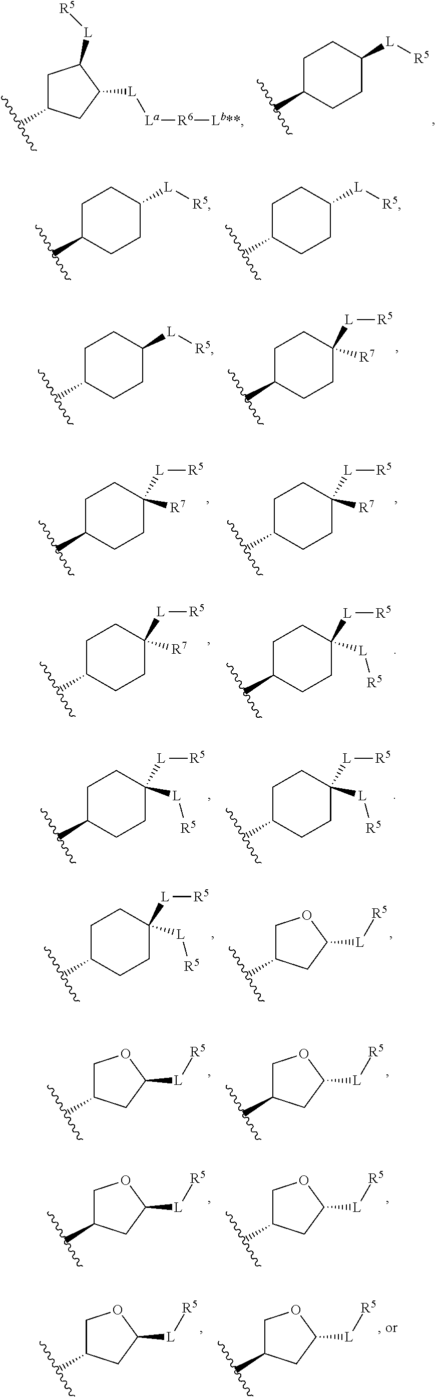

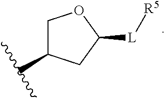

In some embodiments, R² is

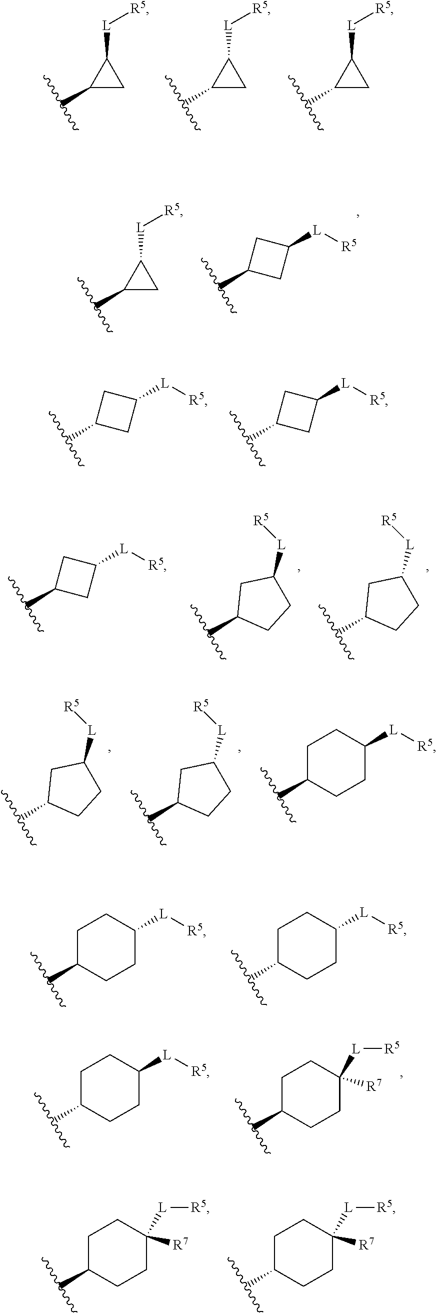

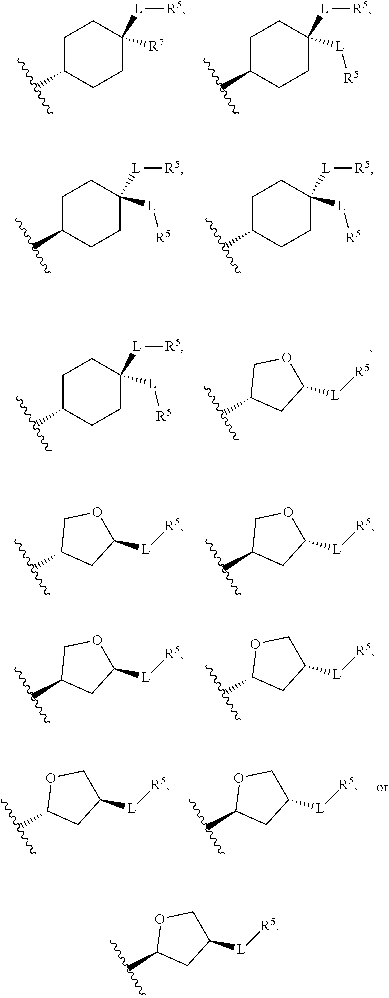

In some embodiments, R² is 5- to 10-membered heteroaryl comprising 1 to 4 heteroatoms selected from N, O, and S, and wherein R² is optionally substituted with one or more instances of -L-R⁵ or R⁷. In some embodiments, R² is 5- to 10-membered heteroaryl comprising 1 to 4 heteroatoms selected from N, O, and S, and wherein R² is substituted with 1-2 instances of -L-R⁵ or R⁷. In some embodiments, R² is 5- to 6-membered heteroaryl comprising 1 to 4 heteroatoms selected from N, O, and S, and wherein R² is optionally substituted with one or more instances of -L-R⁵ or R⁷. In some embodiments, R² is 5- to 6-membered heteroaryl comprising 1 to 4 heteroatoms selected from N, O, and S, and wherein R² is substituted with 1-2 instances of -L-R⁵ or R⁷.

In some embodiments, R² is 5- to 10-membered heteroaryl comprising 1 to 4 heteroatoms selected from N, O, and S, and wherein R² is optionally substituted with one or more instances of -L-R⁵. In some embodiments, R² is 5- to 10-membered heteroaryl comprising 1 to 4 heteroatoms selected from N, O, and S, and wherein R² is optionally substituted with one instance of -L-R⁵. In some embodiments, R² is 5- to 10-membered heteroaryl comprising 1 to 4 heteroatoms selected from N, O, and S. In some embodiments, R² is 5- to 6-membered heteroaryl comprising 1 to 4 heteroatoms selected from N, O, and S, and wherein R² is optionally substituted with one or more instances of -L-R⁵. In some embodiments, R² is 5- to 6-membered heteroaryl comprising 1 to 4 heteroatoms selected from N, O, and S, and wherein R² is optionally substituted with one instance of -L-R⁵.

In some embodiments, R² is C₆-C₁₀ aryl, and wherein R² is optionally substituted with one or more instances of -L-R⁵ or R⁷. In some embodiments, R² is C₆-C₁₀ aryl, and wherein R² is substituted with 1-2 instances of -L-R⁵ or R⁷. In some embodiments, R² is phenyl, and wherein R² is optionally substituted with one or more instances of -L-R⁵ or R⁷. In some embodiments, R² is phenyl, and wherein R² is substituted with 1-2 instances of -L-R⁵ or R⁷. In some embodiments, R² is naphthyl, and wherein R² is optionally substituted with one or more instances of -L-R⁵ or R⁷. In some embodiments, R² is naphthyl, and wherein R² is substituted with 1-2 instances of -L-R⁵ or R⁷.

In some embodiments, R² is C₆-C₁₀ aryl, and wherein R² is optionally substituted with one or more instances of -L-R⁵. In some embodiments, R² is C₆-C₁₀ aryl, and wherein R² is optionally substituted with one instance of -L-R⁵. In some embodiments, R² is phenyl, and wherein R² is optionally substituted with one or more instances of -L-R⁵. In some embodiments, R² is naphthyl, and wherein R² is optionally substituted with one or more instances of -L-R⁵.

As described herein, with respect to any formula provided herein, each L is independently selected from a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—. In some embodiments, each L is independently selected from a bond, —N(R^{a})C(O)O—, —OC(O)N(R^{a})—, —C(O)N(R^{a})—, —N(R^{a})C(O)—, —O—, and —C(O)—. In some embodiments, each L is independently selected from a bond, —N(R^{a})C(O)O—, —OC(O)N(R^{a})—, —C(O)N(R^{a})—, —N(R^{a})C(O)—, —O—, and —C(O)—, wherein each R^{a} of L is independently hydrogen or C₁₋₂ alkyl. In some embodiments, each L is independently selected from —N(R^{a})C(O)O—, —C(O)N(R^{a})—, and —C(O)—. In some embodiments, each L is independently selected from —N(H)C(O)O—, —C(O)NH—, —C(O)N(CH₃)—, and —C(O)—. In some embodiments, each L is independently selected from a bond, —N(R^{a})C(O)O—, and —N(R^{a})C(O)—. In some embodiments, each L is independently selected from a bond, —N(R^{a})C(O)O—, and —N(R^{a})C(O)—, wherein each R^{a} of L is independently hydrogen or C₁₋₂ alkyl. In some embodiments, each L is independently selected from a bond, —N(R^{a})C(O)O—, —OC(O)N(R^{a})—, —C(O)N(R^{a})—, —N(R^{a})C(O)—, —N(R^{a}), —O—, —C(O)—, and —OC(O)—. In some embodiments, each L is independently selected from a bond, —N(R^{a})C(O)O—, —OC(O)N(R^{a})—, —C(O)N(R^{a})—, —N(R^{a})C(O)—, —N(R^{a})—, —O—, —C(O)—, and —OC(O)—, wherein each R^{a} of L is independently hydrogen or C₁₋₂ alkyl. In some embodiments, each L is independently selected from —N(R^{a})C(O)O—, —C(O)N(R^{a})—, —N(R^{a})C(O)—, —N(R^{a})—, —C(O)—, and —OC(O)—. In some embodiments, each L is independently selected —N(H)C(O)O—, —N(CH₃)C(O)O—, —C(O)N(H)—, —C(O)N(CH₃)—, —N(H)C(O)—, —N(H)—, —C(O)—, and —OC(O)—.

In some embodiments, L is a bond.

In some embodiments, L is —N(R^{a})C(O)N(R^{a})—. In some embodiments, L is —NHC(O)NH—.

In some embodiments, L is —N(R^{a})C(O)O—. In some embodiments, L is —N(H)C(O)O—. In some embodiments, L is —N(CH₃)C(O)O—.

In some embodiments, L is —OC(O)N(R^{a})—. In some embodiments, L is —OC(O)NH—.

In some embodiments, L is —C(O)N(R^{a})—. In some embodiments, L is —C(O)NH—. In some embodiments, L is —C(O)N(CH₃)—.

In some embodiments, L is —N(R^{a})C(O)—. In some embodiments, L is —NHC(O)—. In some embodiments, L is —N(C₁₋₃ alkyl)C(O)—. In some embodiments, L is —N(CH₃)C(O)—.

In some embodiments, L is —N(R^{a})—. In some embodiments, L is —NH—.

In some embodiments, L is —O—.

In some embodiments, L is —C(O)—.

In some embodiments, L is —C(O)O—.

In some embodiments, L is —OC(O)—.

In some embodiments, L is —N(R^{a})C(S)O—. In some embodiments, L is —NHC(S)O—.

In some embodiments, L is —OC(S)N(R^{a})—. In some embodiments, L is —OC(S)NH—.

In some embodiments, L is —C(S)N(R^{a})—. In some embodiments, L is —C(S)NH—.

In some embodiments, L is —N(R^{a})C(S)—. In some embodiments, L is —NHC(S)—.

In some embodiments, L is —C(S)—.

In some embodiments, L is —C(S)O—.

In some embodiments, L is —OC(S)—.

As described herein, with respect to any formula provided herein (e.g., any of Formulae I-IX), each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl. In some embodiments, each R⁵ is independently selected from optionally substituted C₁-C₆ aliphatic, optionally substituted 2- to 10-membered heteroaliphatic, optionally substituted 4- to 10-membered heterocycle comprising 1 to 4 heteroatoms selected from N, O, and S, and optionally substituted 5- to 10-membered heteroaryl comprising 1 to 4 heteroatoms selected from N, O, and S. In some embodiments, each R⁵ is independently selected from optionally substituted C₁-C₆ aliphatic, optionally substituted C₃-C₁₂ cycloaliphatic, optionally substituted 4- to 10-membered heterocycle comprising 1 to 4 heteroatoms selected from N, O, and S, and optionally substituted 5- to 10-membered heteroaryl comprising 1 to 4 heteroatoms selected from N, O, and S. In some embodiments, each R⁵ is independently selected from optionally substituted C₁-C₆ aliphatic, optionally substituted C₃-C₆ cycloaliphatic, optionally substituted 4- to 6-membered heterocycle comprising 1 to 2 heteroatoms selected from N, O, and S, and optionally substituted 5- to 6-membered heteroaryl comprising 1 to 4 heteroatoms selected from N, O, and S.

In some embodiments, R⁵ is optionally substituted C₁-C₆ aliphatic. In some embodiments, R⁵ is optionally substituted C₁-C₃ aliphatic. In some embodiments, R⁵ is C₁-C₆ aliphatic (e.g., —CH₃ or —CH₂CH₃). In some embodiments, R⁵ is C₁-C₃ aliphatic (e.g., —CH₃ or —CH₂CH₃). In some embodiments, R⁵ is —CH₃. In some embodiments, R⁵ is —CH₂CH₃. In some embodiments, R⁵ is C₁-C₆ aliphatic optionally substituted with two occurrences of —(CH₂)₀₋₄R^{∘}, wherein the two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur. In some embodiments, R⁵ is C₁-C₆ aliphatic optionally substituted with two occurrences of —(CH₂)₀₋₄R^{∘}, wherein the two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted with halogen, —(CH₂)₀₋₂R^{●}, -(haloR^{●}), —(CH₂)₀₋₂OH, or =O (“oxo”), and wherein each R^{●} is unsubstituted or where preceded by “halo” is substituted only with one or more halogens, and is independently selected from C₁₋₄ aliphatic. In some embodiments, R⁵ is optionally substituted C₂-C₆ alkenyl. In some embodiments, R⁵ is —CH═CH₂. In some embodiments, R⁵ is C₁-C₆ aliphatic, optionally substituted with —CN, —OR^{∘}, halogen, or —(CH₂)₀₋₄N(R^{∘})C(O)R^{∘}. In some embodiments, R⁵ is C₁-C₆aliphatic, optionally substituted with —CN, —OH, halogen, or —CH₂—N(H)C(O)(C₁-C₆ aliphatic). In some embodiments, R⁵ is C₁-C₆ aliphatic, optionally substituted with —CN, —OH, halogen, or —N(H)C(O)(C₁-C₆ aliphatic). In some embodiments, R⁵ is C₁-C₆ aliphatic, optionally substituted with —CN, —OH, halogen, —N(H)C(O)(C₁-C₆ aliphatic), —N(CH₃)C(O)(C₁-C₆ aliphatic), —N(CH₃)C(O)(3-membered cycloaliphatic), —N(CH₃)C(O)(C₁-C₆ aliphatic)(4-membered heterocycle), or —N(CH₃)C(O)(C₁-C₆ aliphatic)(5-membered heteroaryl). In some embodiments, R⁵ is C₁-C₆ aliphatic, optionally substituted with R^{∘}, —CN, —OR^{∘}, or —N(R^{∘})C(O)R^{∘}. In some embodiments, R⁵ is C₁-C₆ aliphatic, optionally substituted with R^{∘}, —CN, —OR^{∘}, or —N(R^{∘})C(O)R^{∘}, wherein each R^{∘} is independently hydrogen, C₁₋₆ aliphatic, or a 3- to 6-membered saturated, partially unsaturated, or aryl ring having 0-4 heteroatoms independently selected from nitrogen, oxygen, or sulfur. In some embodiments, R⁵ is C₁-C₆ aliphatic, optionally substituted with —CN, —OH, —O(C₁₋₃ alkyl), —N(H)C(O)(C₁₋₃ alkyl), —N(C₁₋₃ alkyl)C(O)(C₁₋₃ alkyl), or a 5- to 6-membered heterocycle having 1-2 heteroatoms independently selected from nitrogen, oxygen, or sulfur. In some embodiments, R⁵ is C₁-C₆ aliphatic, optionally substituted with —CN, —OH, —O(C₁₋₃ alkyl), —N(H)C(O)(C₁₋₃ alkyl), —N(C₁₋₃ alkyl)C(O)(C₁₋₃ alkyl), —N(C₁₋₃ alkyl)C(O)(C₁₋₃ aliphatic), —N(C₁₋₃ alkyl)C(O)(3-membered cycloaliphatic), —N(C₁₋₃ alkyl)C(O)(C₁₋₃ aliphatic)(4-membered heterocycle), —N(C₁₋₃ alkyl)C(O)(C₁₋₃ aliphatic)(5-membered heteroaryl), or two occurrences of —(CH₂)₀₋₄R^{∘}, wherein the two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted with halogen, —(CH₂)₀₋₂R^{●}, -(haloR^{●}), —(CH₂)₀₋₂OH, or =O (“oxo”), and wherein each R^{●} is unsubstituted or where preceded by “halo” is substituted only with one or more halogens, and is independently selected from C₁₋₄ aliphatic.

In some embodiments, R⁵ is

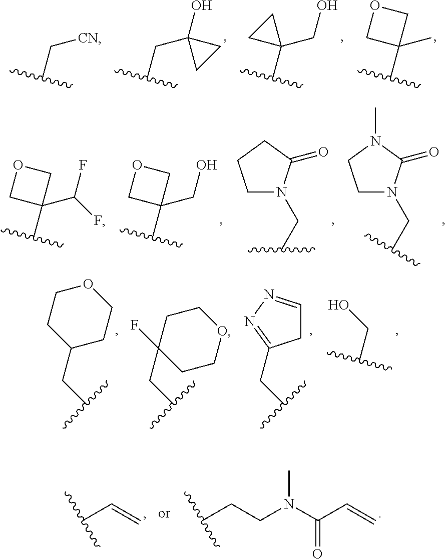

In some embodiments, R⁵ is —CH₃, —CH₂CN, —CH₂OH, —CH₂OCH₃, —CH₂CH₃,

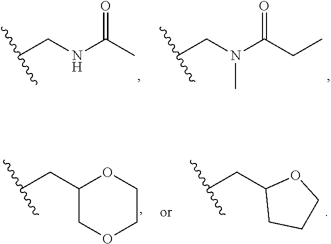

In some embodiments, R⁵ is —CH₃, —CH₂CN, —CH₂OH, —CH₂OCH₃, —CH₂CH₃,

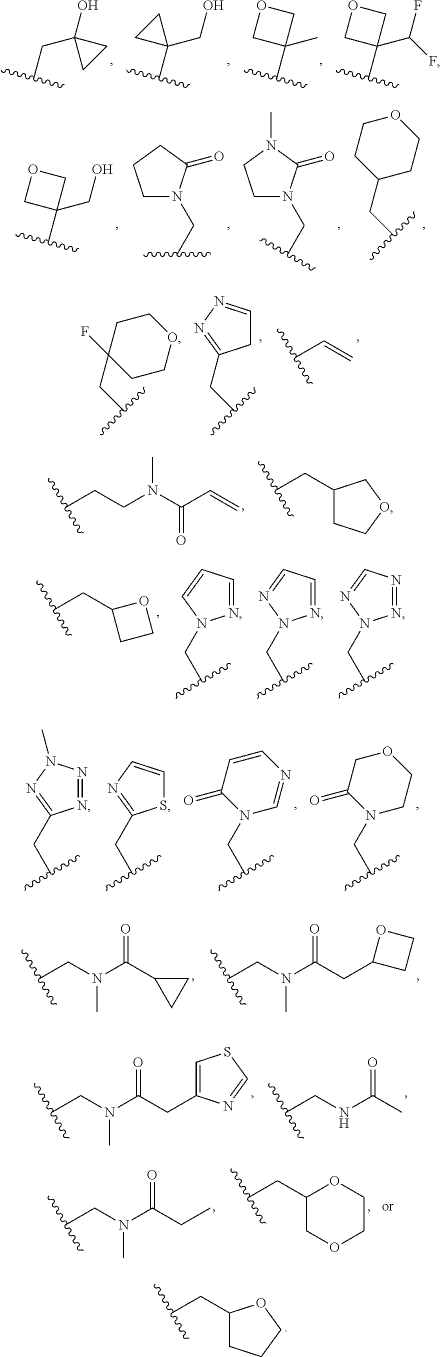

In some embodiments, R⁵ is optionally substituted 2- to 10-membered heteroaliphatic. In some embodiments, R⁵ is 2- to 10-membered heteroaliphatic optionally substituted with two occurrences of —(CH₂)₀₋₄R^{∘}, wherein the two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur. In some embodiments, R⁵ is 2- to 10-membered heteroaliphatic optionally substituted with one or more —(CH₂)₀₋₄R^{∘} or =O (“oxo”), wherein each R^{∘} is independently hydrogen, C₁₋₆ aliphatic, or a 3- to 6-membered saturated, partially unsaturated, or aryl ring having 0-4 heteroatoms independently selected from nitrogen, oxygen, or sulfur, or two occurrences of —(CH₂)₀₋₄R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur. In some embodiments, R⁵ is

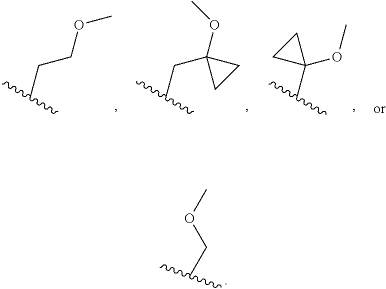

In some embodiments, R⁵ is

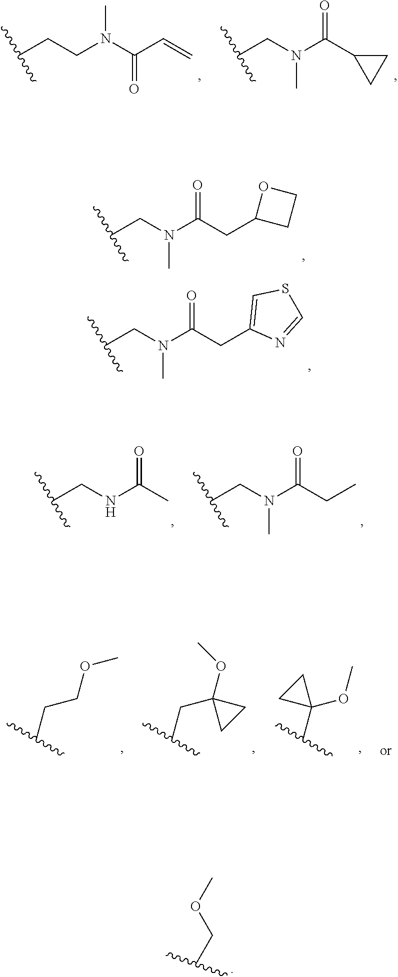

In some embodiments, R⁵ is optionally substituted C₃-C₁₂ cycloaliphatic. In some embodiments, R⁵ is optionally substituted C₃-C₆ cycloaliphatic. In some embodiments, R⁵ is optionally substituted C₃-C₆ cycloaliphatic selected from cyclopropyl and cyclobutyl. In some embodiments, R⁵ is optionally substituted C₃-C₆ cycloaliphatic selected from cyclopropyl, cyclobutyl, and cyclohexyl. In some embodiments, R⁵ is C₃-C₆ cycloaliphatic optionally substituted with —(CH₂)₀₋₄N(R^{∘})C(O)OR^{∘}, wherein the first instance of R^{∘} is hydrogen and the second instance of R^{∘} is —(CH₂)₀₋₂R^{●}, wherein R^{●} is C₁₋₄ aliphatic. In some embodiments, R⁵ is C₃-C₆ cycloaliphatic optionally substituted with —(CH₂)₀₋₄N(R^{∘})C(O)OR^{∘}, wherein the first instance of R^{∘} is hydrogen and the second instance of R^{∘} is C₁₋₆ aliphatic. In some embodiments, R⁵ is C₃-C₆ cycloaliphatic optionally substituted with

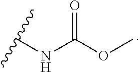 [SMILES: *NC(=O)OC]

In some embodiments, R⁵ is C₃-C₆ cycloaliphatic optionally substituted with one or more halogen or —N(R^{∘})C(O)R^{∘}. In some embodiments, R⁵ is C₃-C₆ cycloaliphatic optionally substituted with one or more halogen or —N(R^{∘})C(O)R^{∘}, wherein each R^{∘} is independently hydrogen or C₁₋₆ aliphatic. In some embodiments, R⁵ is cyclopropyl optionally substituted with 1-2 fluoro. In some embodiments, R⁵ is C₃-C₆ cycloaliphatic optionally substituted with one or more halogen, —(CH₂)₀₋₄N(R^{∘})C(O)OR^{∘}, —N(R^{∘})C(O)R^{∘}, —CN, —(CH₂)₀₋₄R^{∘}, or —(CH₂)₀₋₄OR^{∘}. In some embodiments, R⁵ is C₃-C₆ cycloaliphatic optionally substituted with one or more halogen, —(CH₂)₀₋₄N(R^{∘})C(O)OR^{∘}, —N(R^{∘})C(O)R^{∘}, —CN, —(CH₂)₀₋₄R^{∘}, or —(CH₂)₀₋₄OR^{∘}, wherein each R^{∘} is independently selected from hydrogen, C₁₋₆ aliphatic or an optionally substituted 5-membered aryl ring having 0-4 heteroatoms independently selected from nitrogen, oxygen, or sulfur, wherein the 5-membered aryl ring having 0-4 heteroatoms independently selected from nitrogen, oxygen, or sulfur is optionally substituted with —(CH₂)₀₋₂R^{●}, wherein R^{●} is C₁₋₄ aliphatic. In some embodiments, R⁵ is

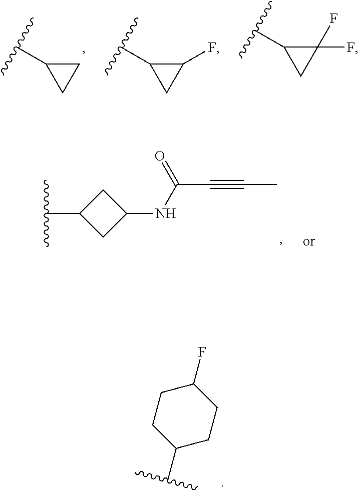

In some embodiments, R⁵ is

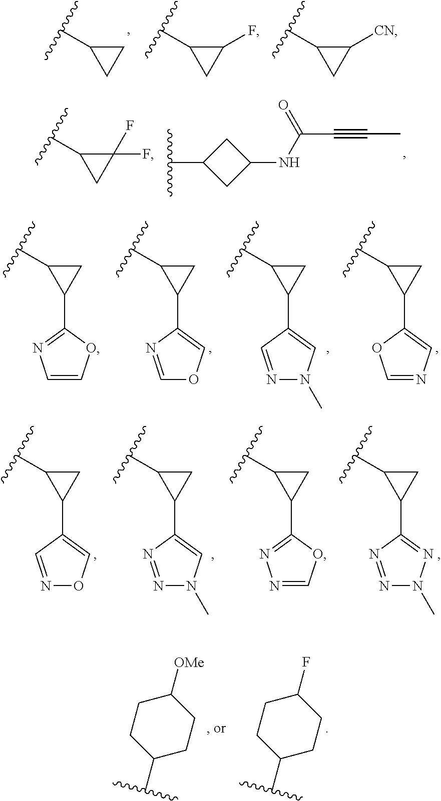

In some embodiments, R⁵ is

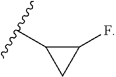 [SMILES: *C1CC1F]

In some embodiments, R⁵ is optionally substituted 4- to 10-membered heterocycle comprising 1 to 4 heteroatoms selected from N, O, and S. In some embodiments, R⁵ is optionally substituted monocyclic 4- to 10-membered heterocycle comprising 1 to 4 heteroatoms selected from N, O, and S. In some embodiments, R⁵ is optionally substituted monocyclic 4- to 6-membered heterocycle comprising 1 to 4 heteroatoms selected from N, O, and S. In some embodiments, R⁵ is optionally substituted monocyclic 4- to 6-membered heterocycle comprising 1 to 2 heteroatoms selected from N, O, and S. In some embodiments, R⁵ is monocyclic 4- to 6-membered heterocycle comprising 1 to 2 heteroatoms selected from N, O, and S, wherein the heterocycle is optionally substituted with one or more oxo or —OR^{∘}, wherein each R^{∘} is independently hydrogen or C₁₋₆ aliphatic. In some embodiments, R⁵ is monocyclic 4- to 6-membered heterocycle comprising 1 to 2 heteroatoms selected from N, O, and S, wherein the heterocycle is optionally substituted with one or more oxo or —(CH₂)₀₋₄OR^{∘}, wherein each R^{∘} is independently hydrogen or C₁₋₆ aliphatic. In some embodiments, R⁵ is optionally substituted spirocyclic 7- to 10-membered heterocycle comprising 1 to 4 heteroatoms selected from N, O, and S. In some embodiments, R⁵ is optionally substituted bicyclic 7- to 10-membered heterocycle comprising 1 to 4 heteroatoms selected from N, O, and S. In some embodiments, R⁵ is

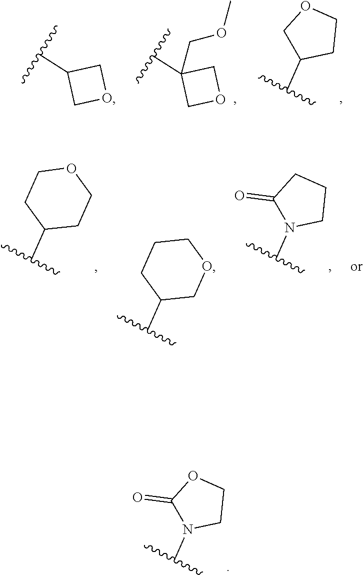

In some embodiments, R⁵ is

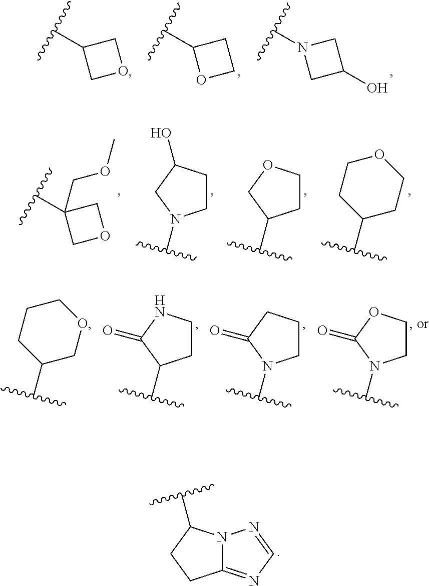

In some embodiments, R⁵ is optionally substituted 5- to 10-membered heteroaryl comprising 1 to 4 heteroatoms selected from N, O, and S. In some embodiments, R⁵ is optionally substituted bicyclic 8- to 10-membered heteroaryl comprising 1 to 4 heteroatoms selected from N, O, and S. In some embodiments, R⁵ is optionally substituted monocyclic 5- to 6-membered heteroaryl comprising 1 to 4 heteroatoms selected from N, O, and S. In some embodiments, R⁵ is monocyclic 5- to 6-membered heteroaryl comprising 1 to 4 heteroatoms selected from N, O, and S. In some embodiments, R⁵ is optionally substituted triazole

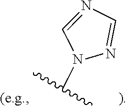 [SMILES: *n1cncn1]

In some embodiments, R⁵ is

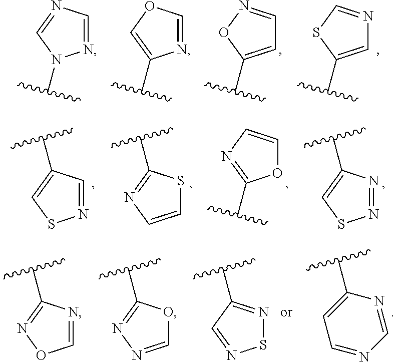

In some embodiments, R⁵ is optionally substituted C₆-C₁₀ aryl. In some embodiments, R⁵ is optionally substituted phenyl. In some embodiments, R⁵ is optionally substituted naphthyl.

In some embodiments, -L-R⁵ is:

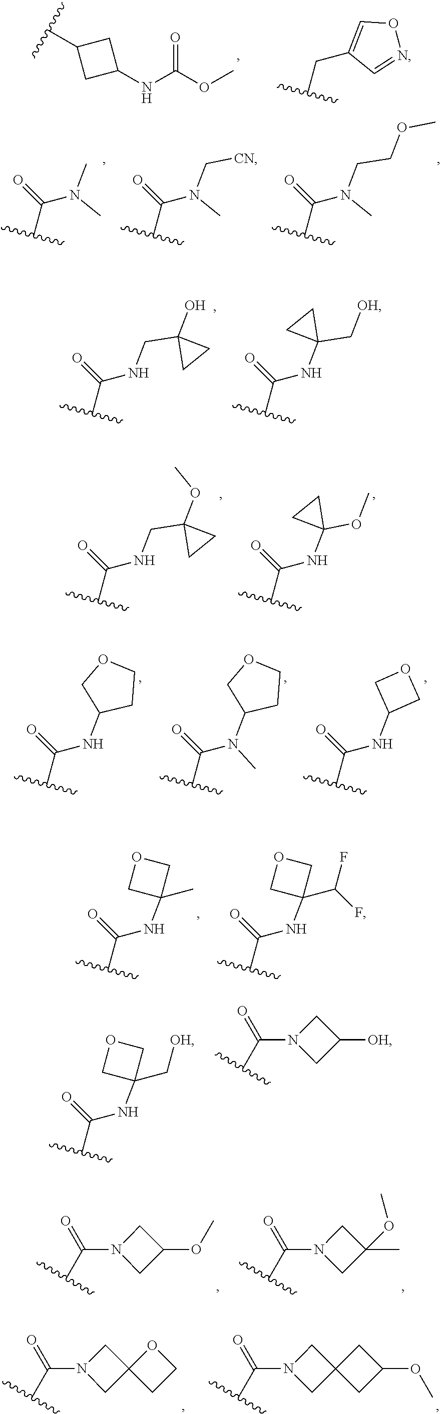

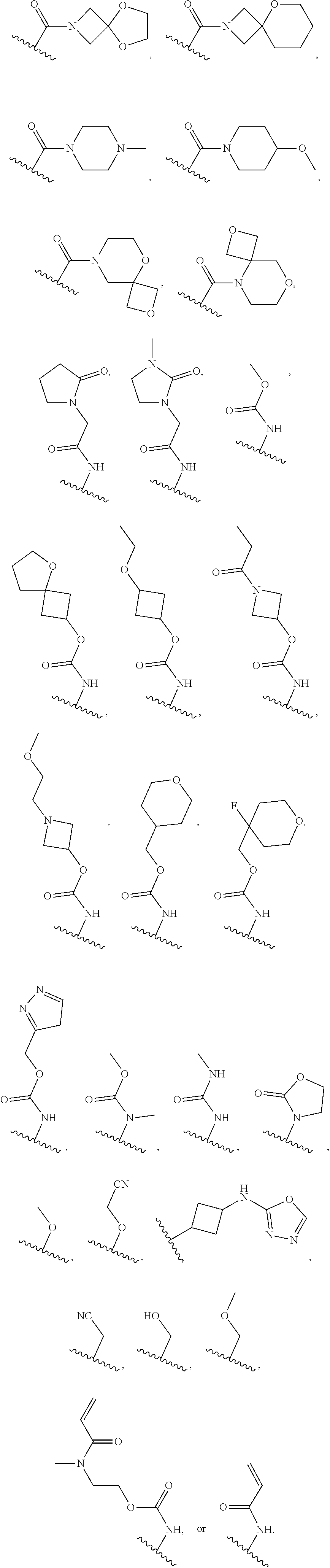

In some embodiments, -L-R⁵ is selected from:

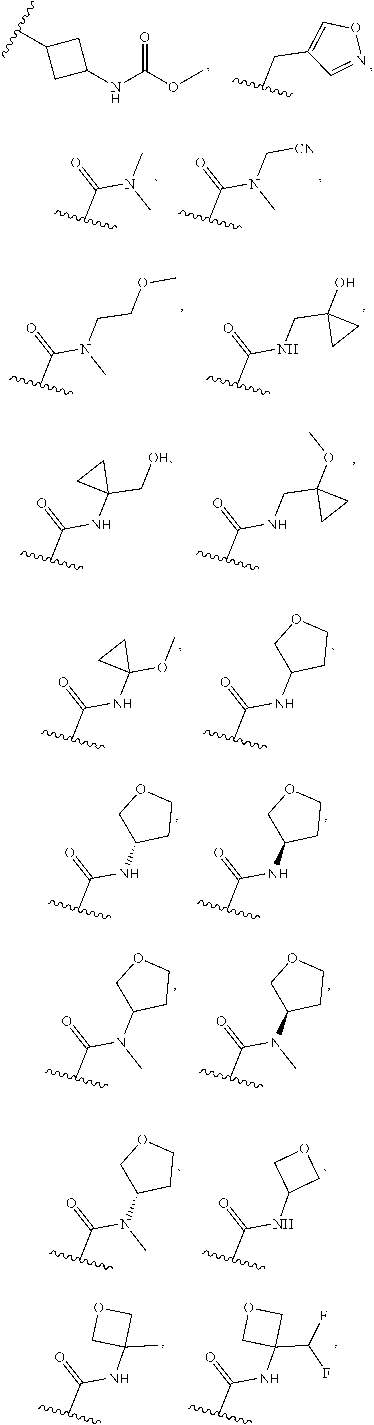

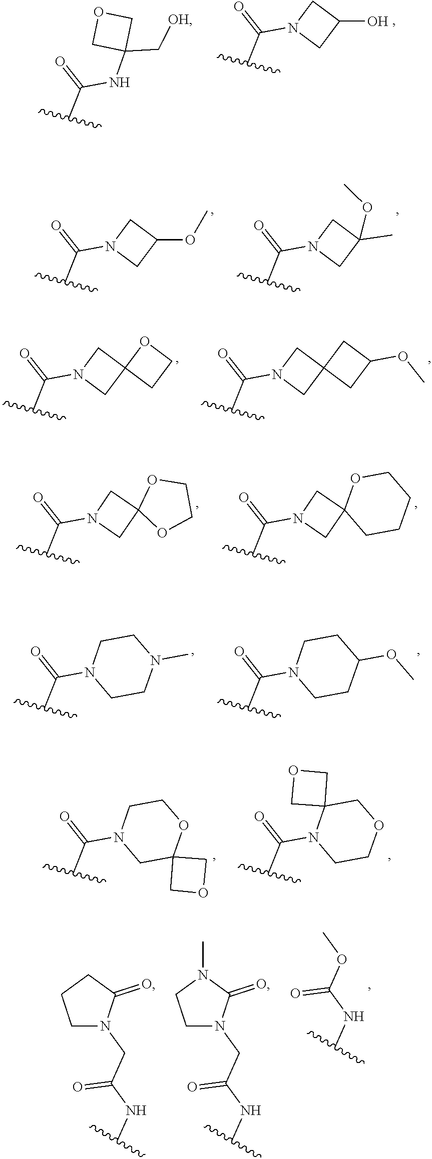

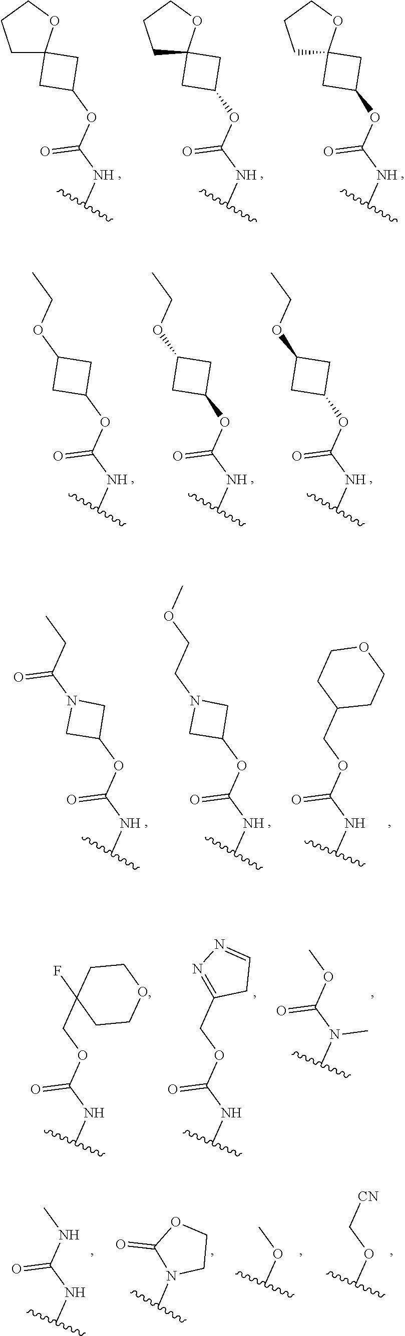

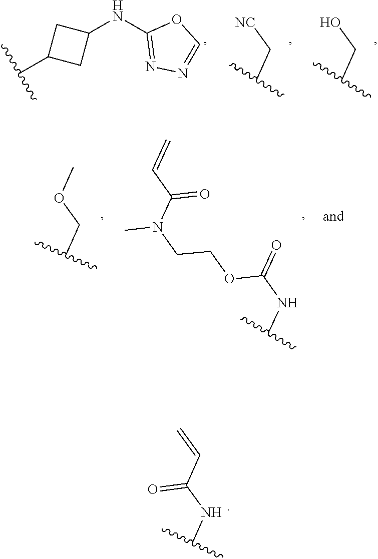

In some embodiments, each -L-R⁵ is independently selected from: —CH₃, —CH₂OH, —CH₂OCH₃,

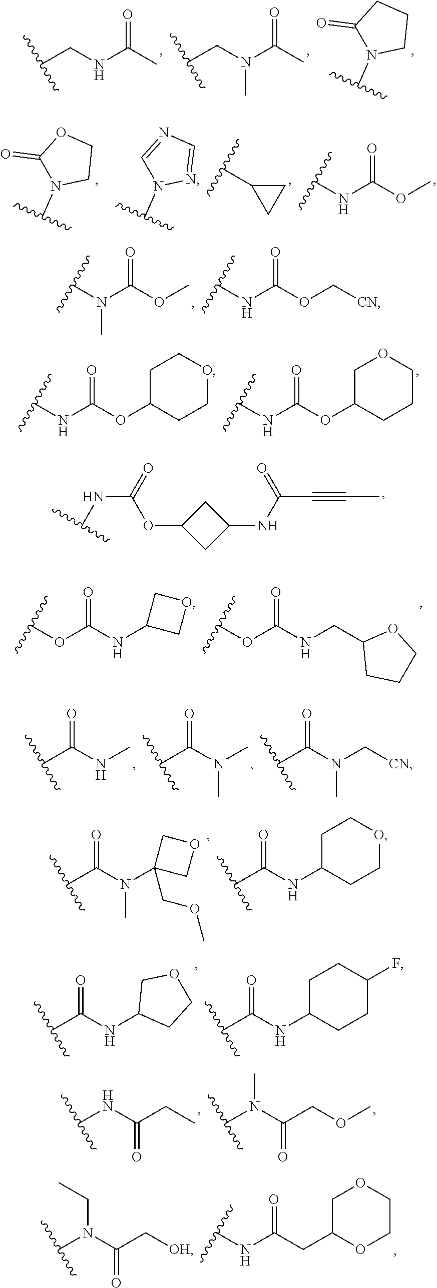

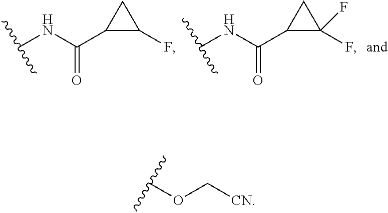

In some embodiments, each -L-R⁵ is independently selected from: —CH₃, —CH₂OH, —CH₂OCH₃,

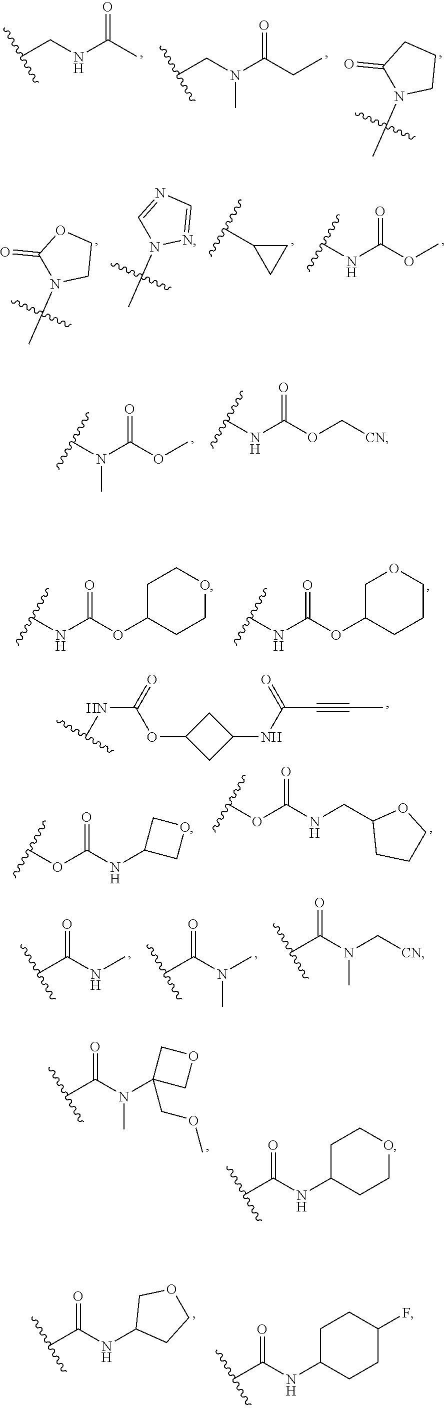

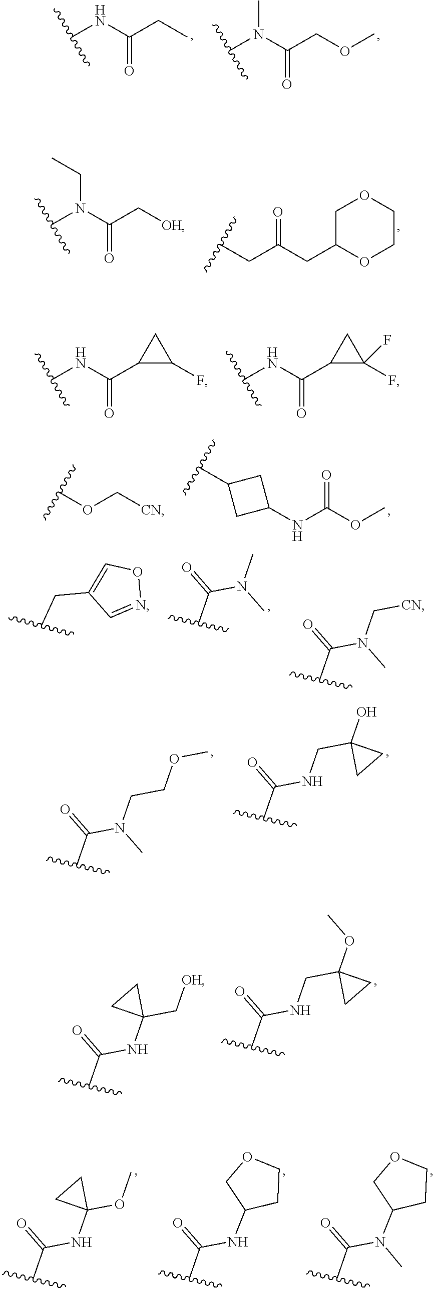

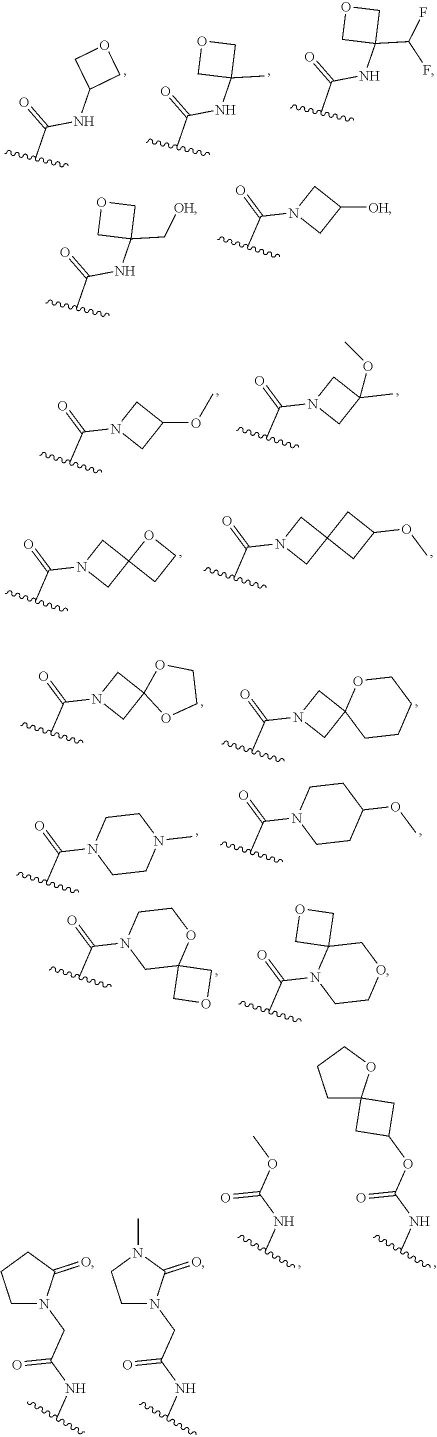

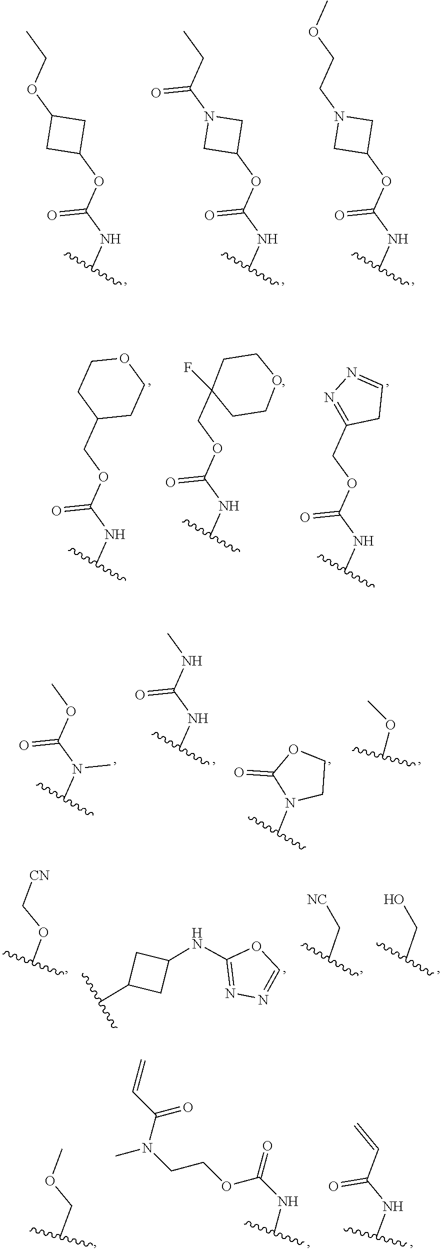

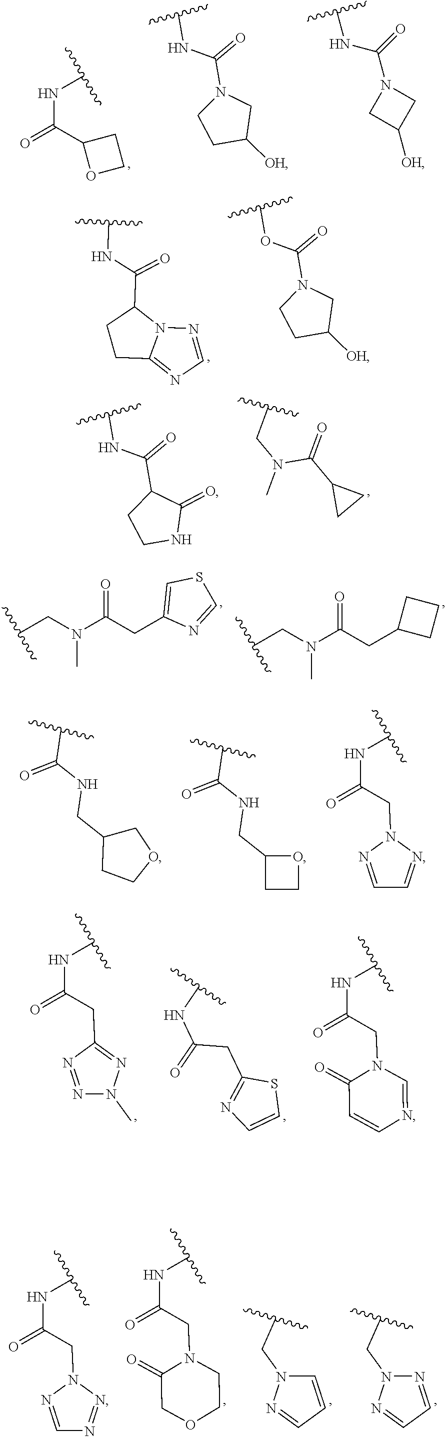

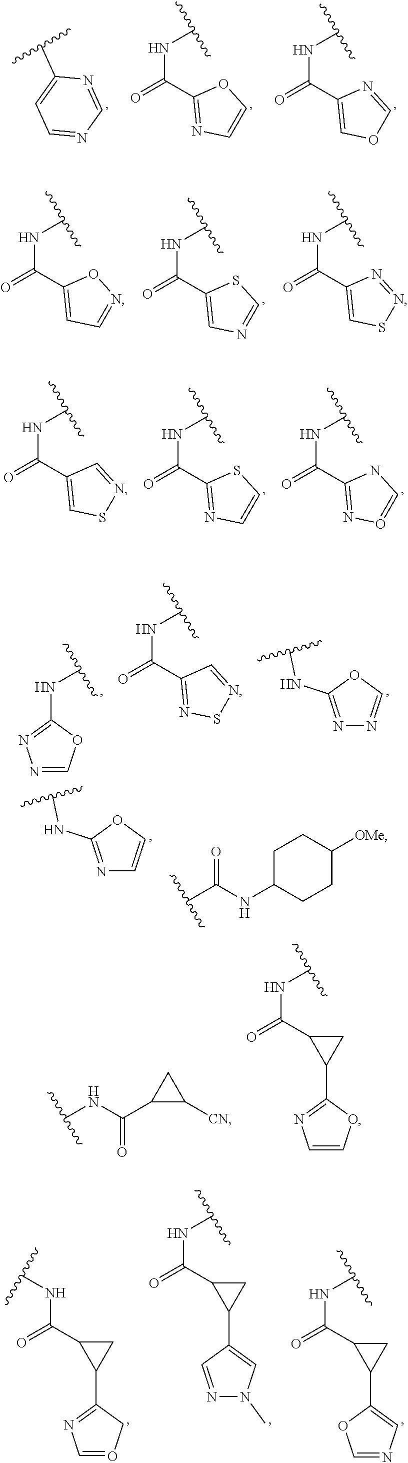

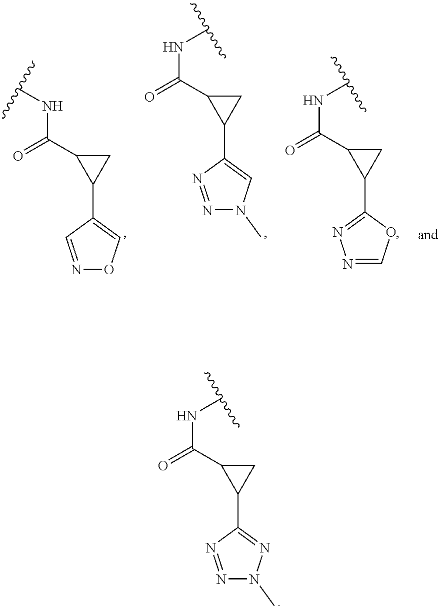

In some embodiments, each -L-R⁵ is independently selected from: —CH₃, —CH₂OH, —CH₂OCH₃,

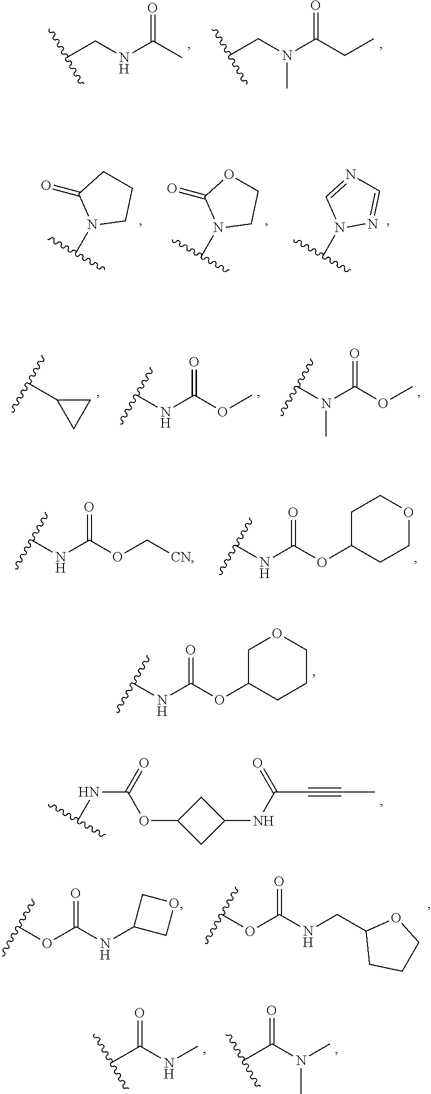

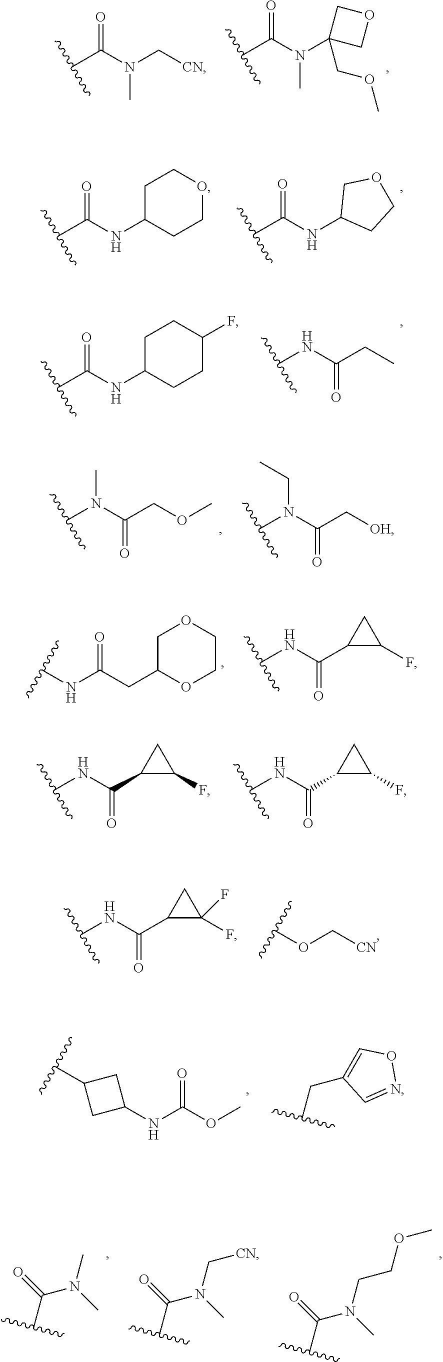

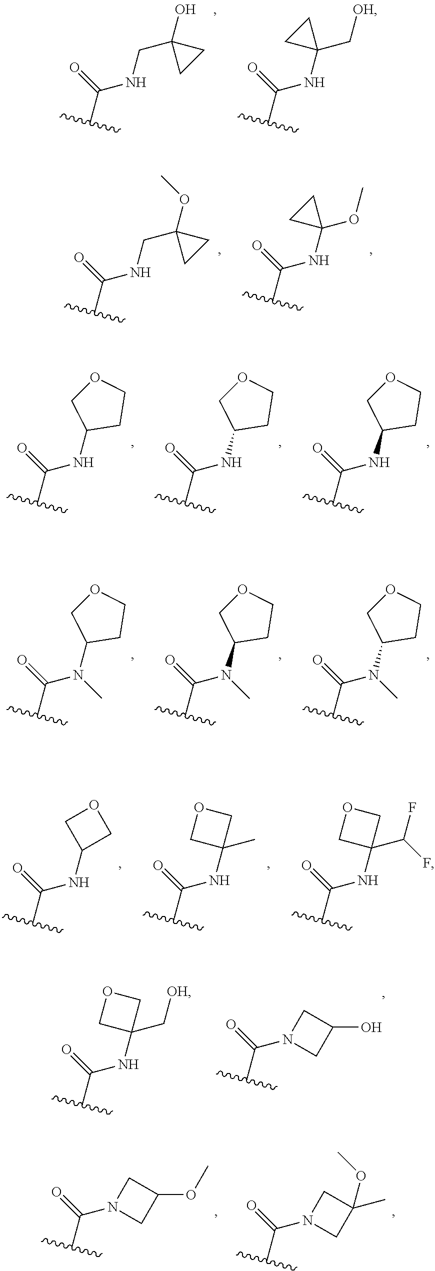

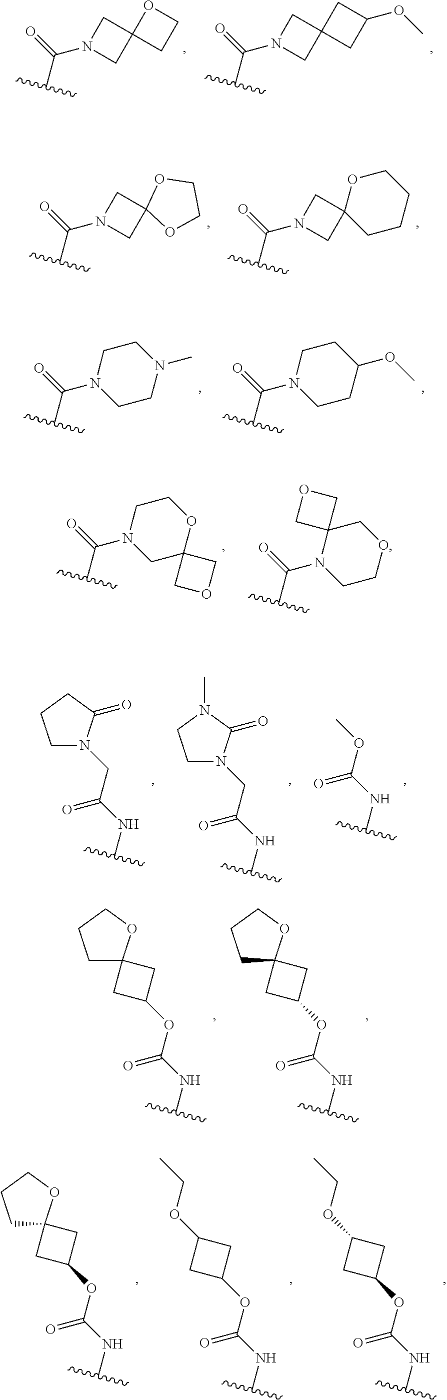

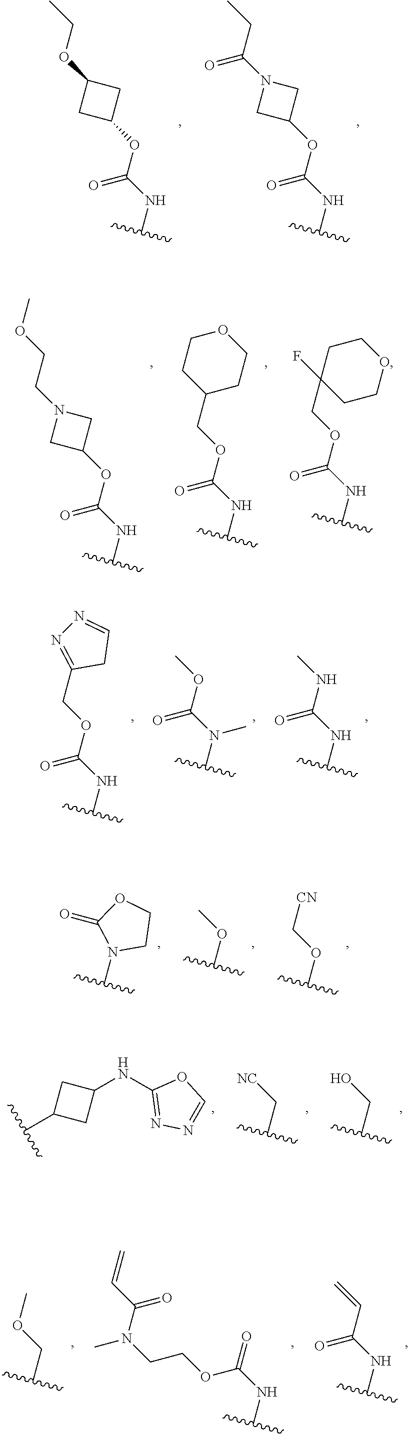

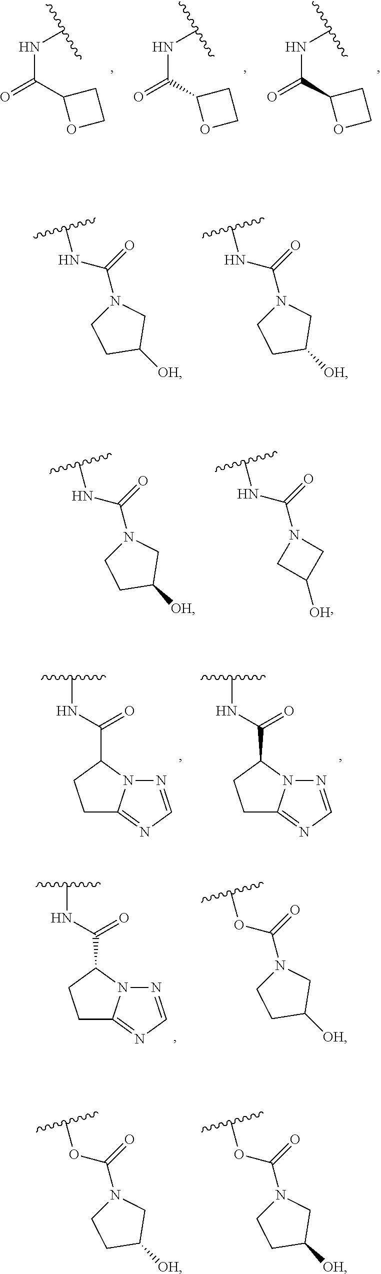

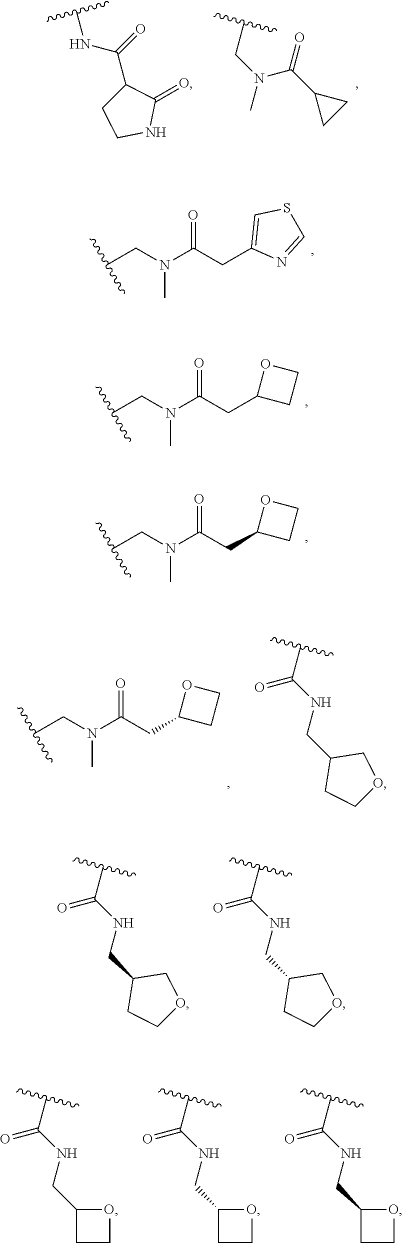

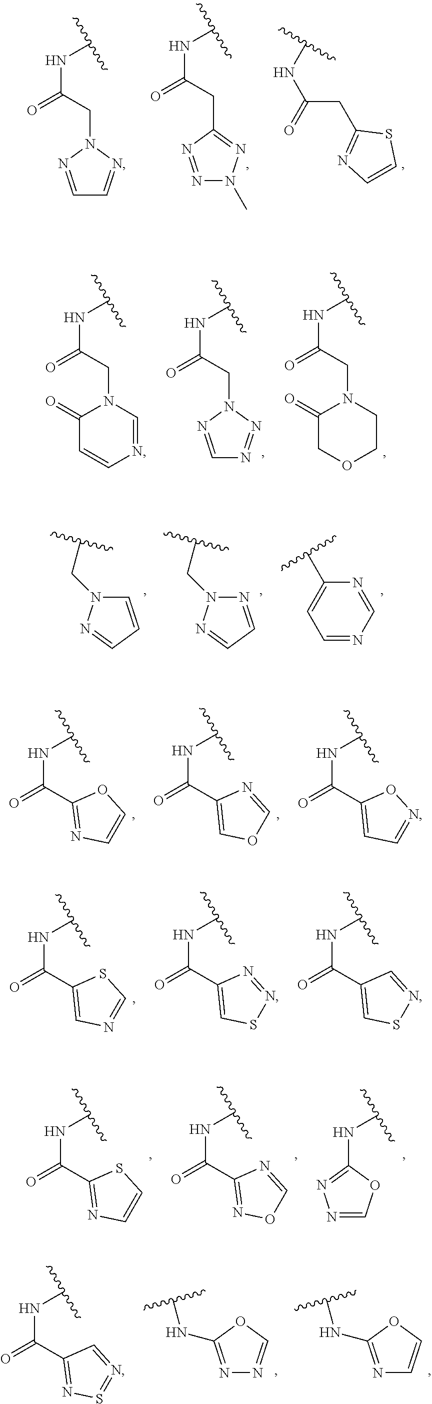

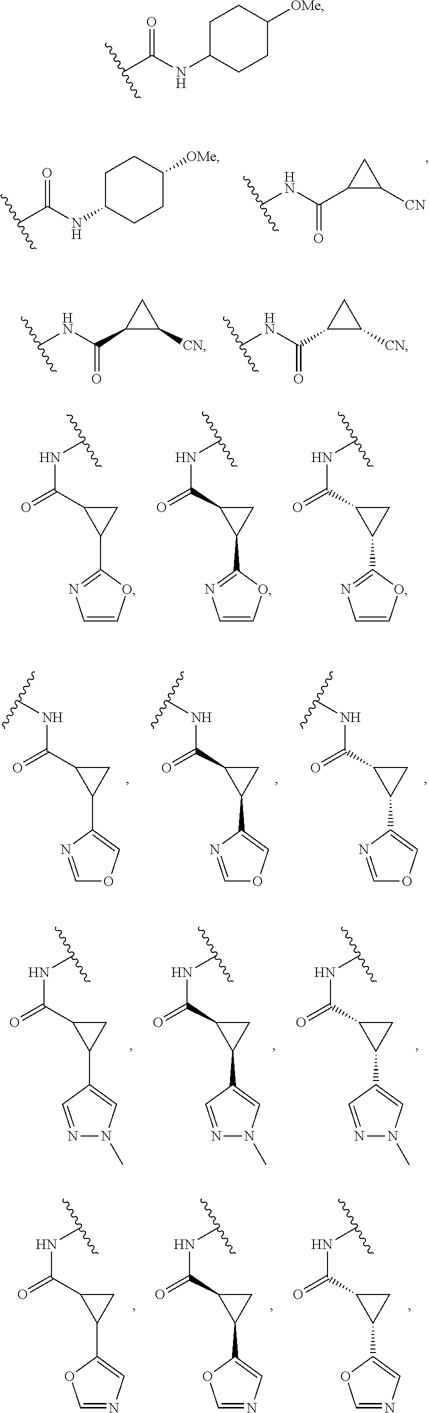

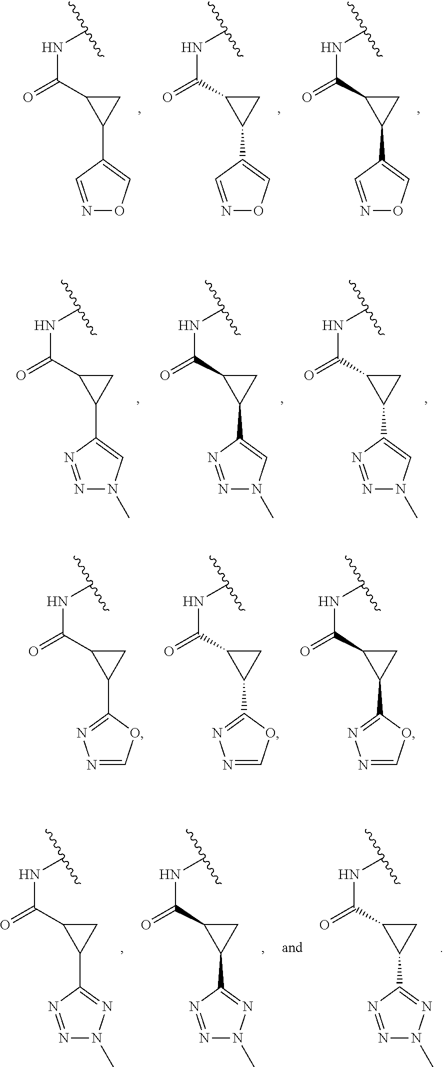

In some embodiments, each -L-R⁵ is

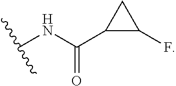 [SMILES: *NC(=O)C1CC1F]

In some embodiments, each -L-R⁵ is

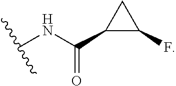 [SMILES: *NC(=O)[C@H]1C[C@H]1F]

In some embodiments, each -L-R⁵ is

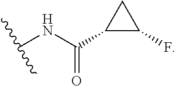 [SMILES: *NC(=O)[C@@H]1C[C@@H]1F]

As described herein, with respect to any formula provided herein (e.g., any of Formulae I-IX), each R⁷ is independently selected from the group consisting of halogen, —OH, —SH, and —NH₂. In some embodiments, each R⁷ is independently selected from the group consisting of —OH, —SH, and —NH₂.

In some embodiments, R⁷ is halogen.

In some embodiments, R⁷ is —OH.

In some embodiments, R⁷ is —SH.

In some embodiments, R⁷ is —NH₂.

In some embodiments, R² that is substituted with one or more -L-R⁵ is:

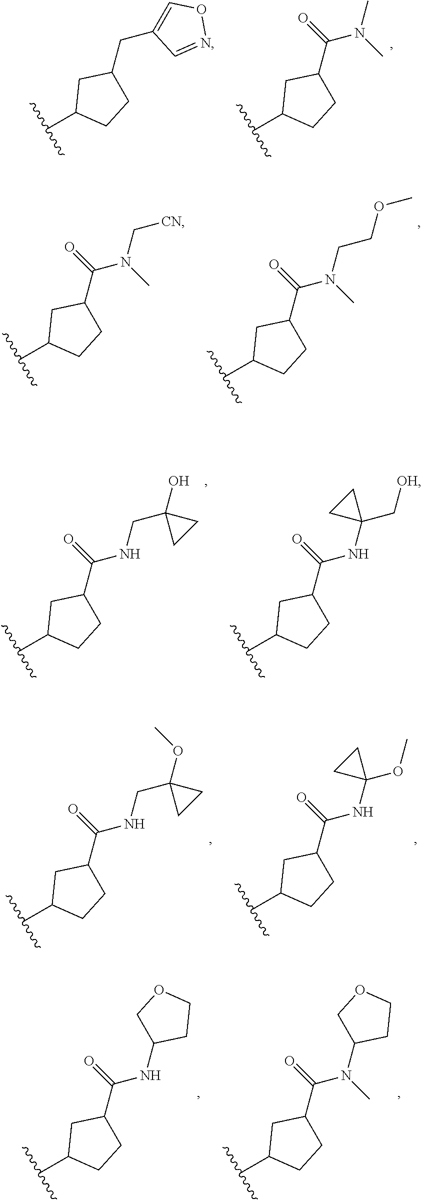

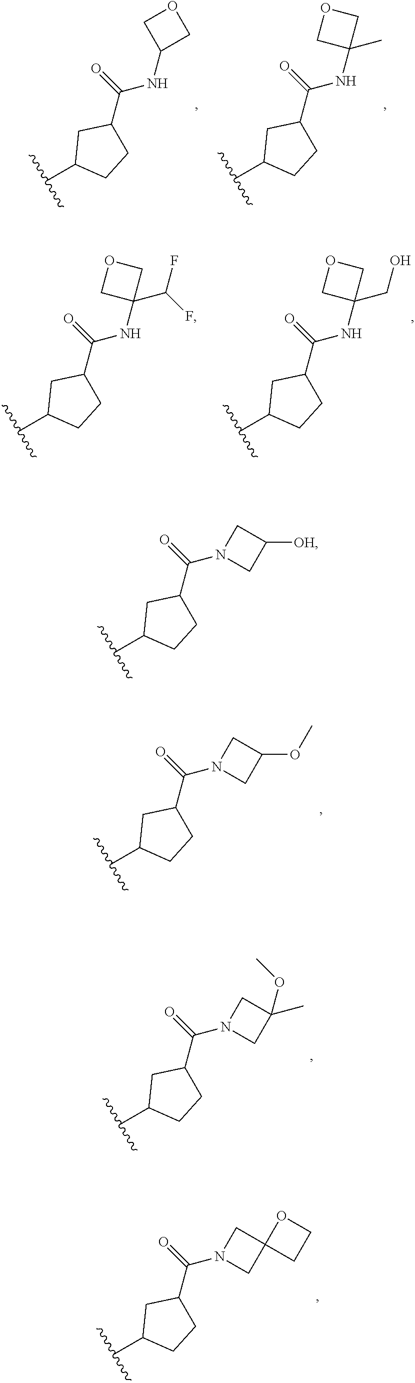

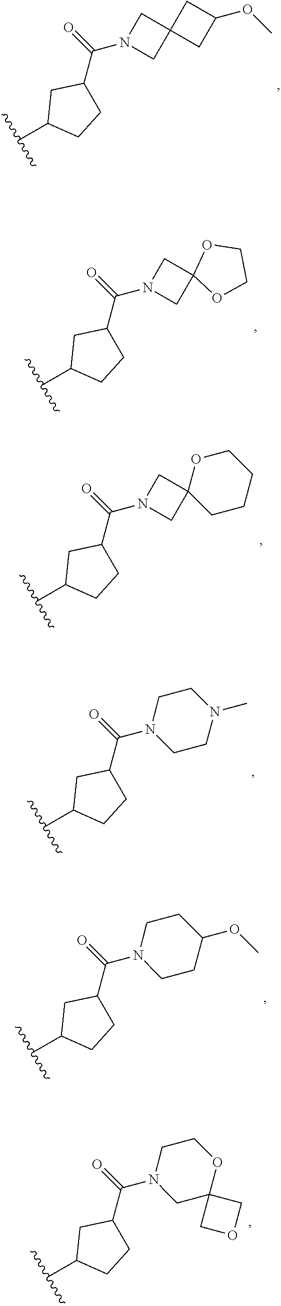 [SMILES: *C1CCC(C(=O)N2CC3(CC(OC)C3)C2)C1 ; *C1CCC(C(=O)N2CC3(C2)OCCO3)C1 ; *C1CCC(C(=O)N2CC3(CCCCO3)C2)C1 ; *C1CCC(C(=O)N2CCN(C)CC2)C1 ; *C1CCC(C(=O)N2CCC(OC)CC2)C1 ; *C1CCC(C(=O)N2CCOC3(COC3)C2)C1]

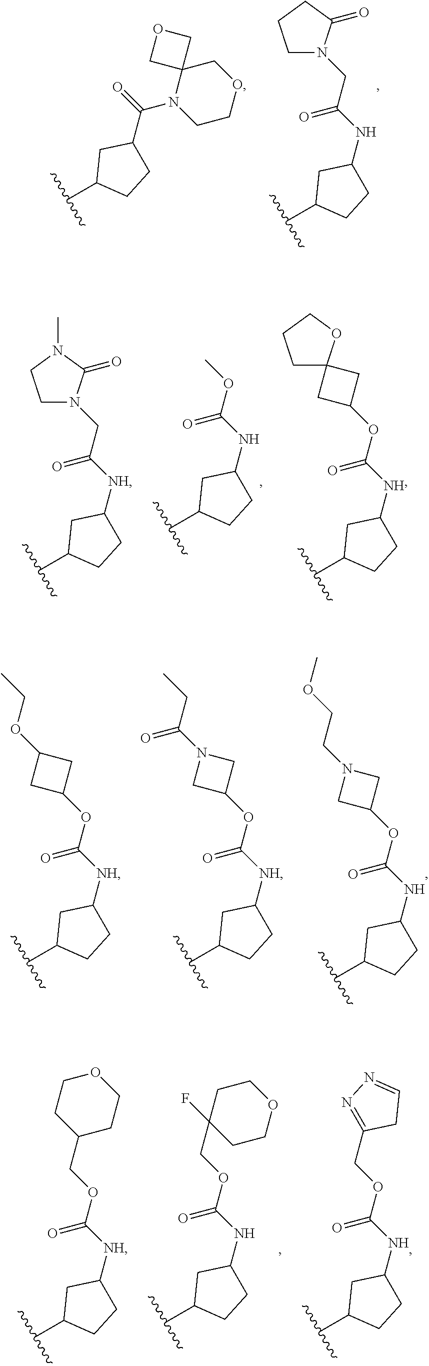

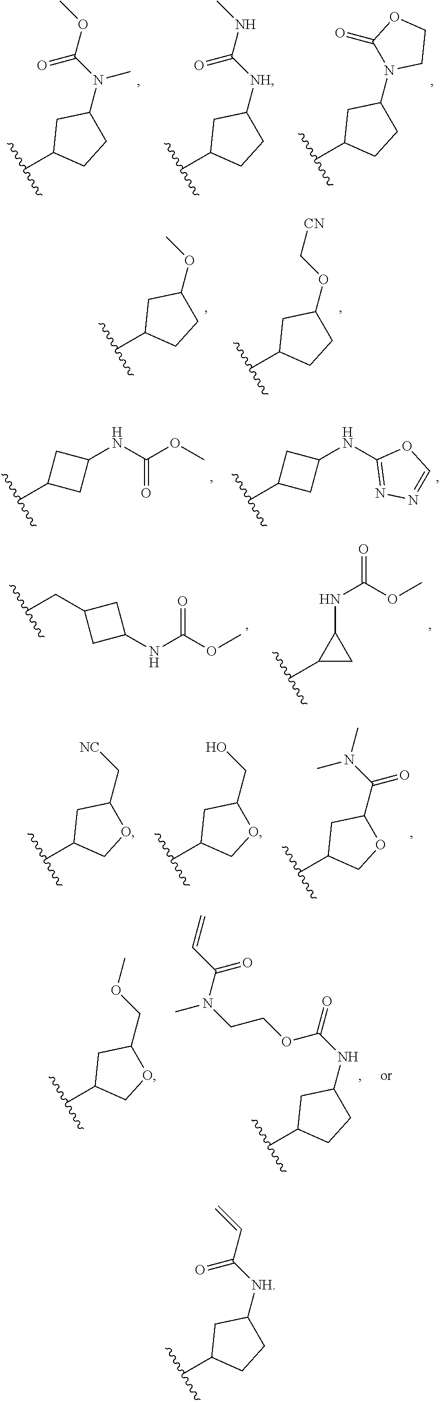

In some embodiments, R² that is substituted with one or more -L-R⁵ is:

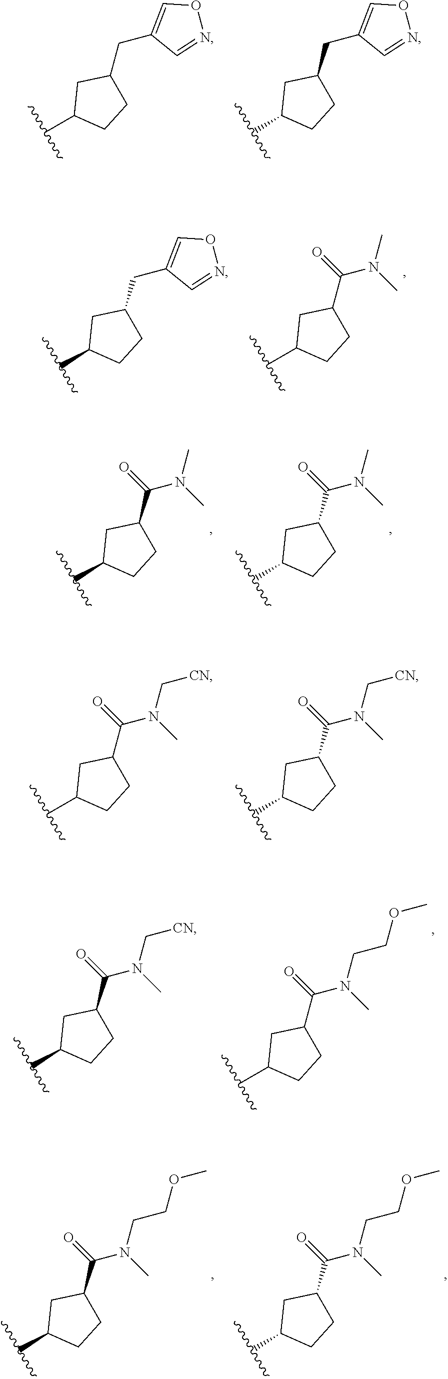

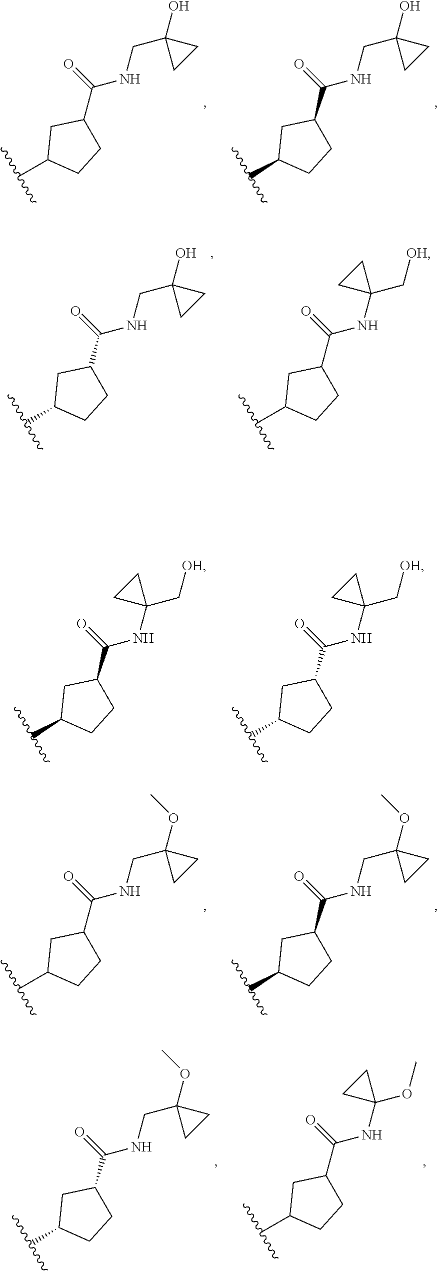

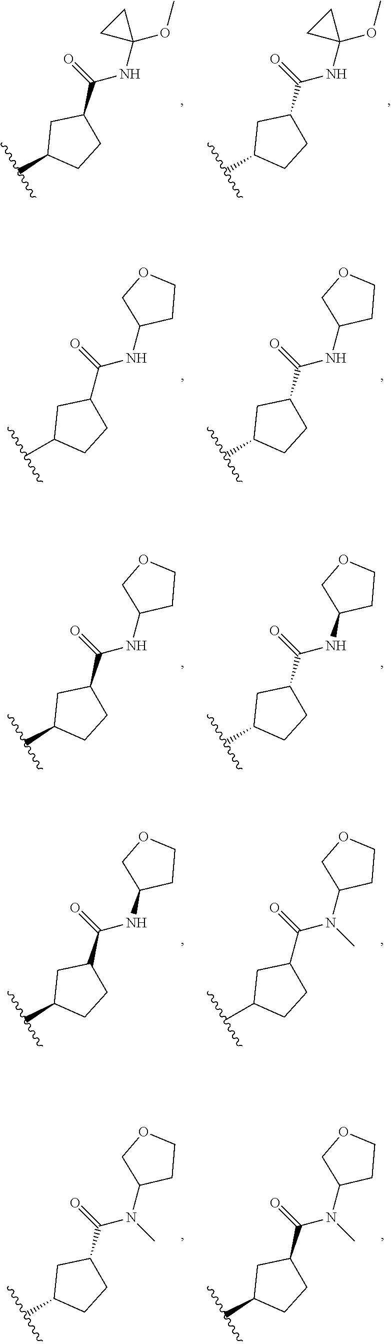

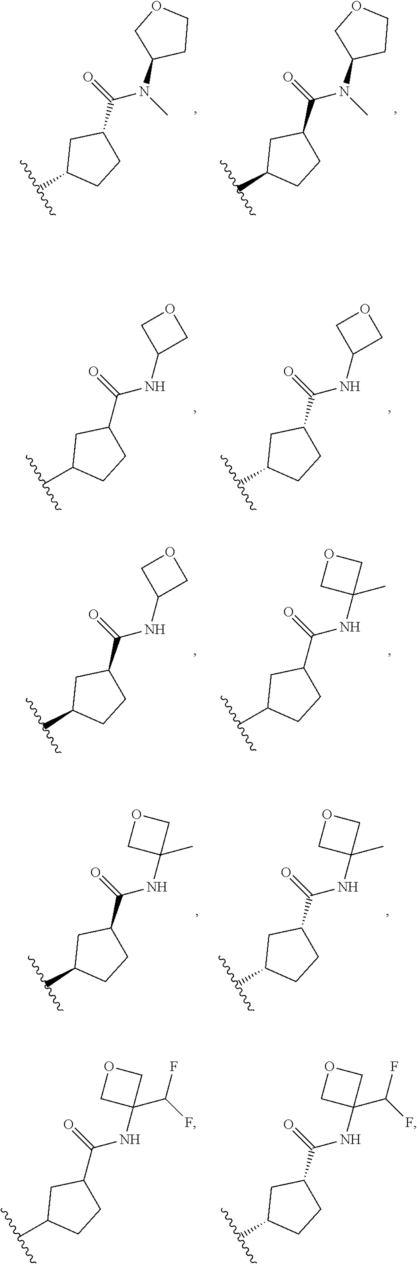

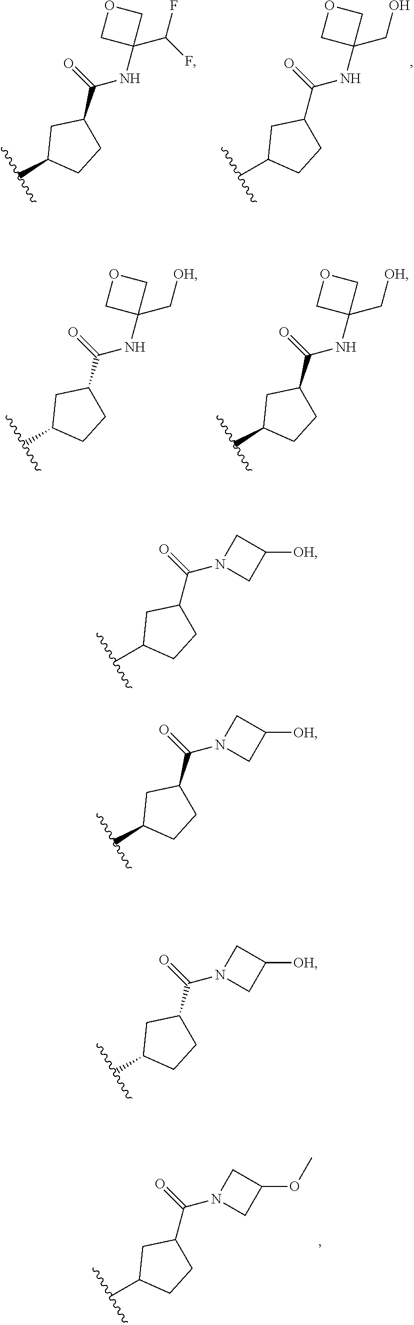

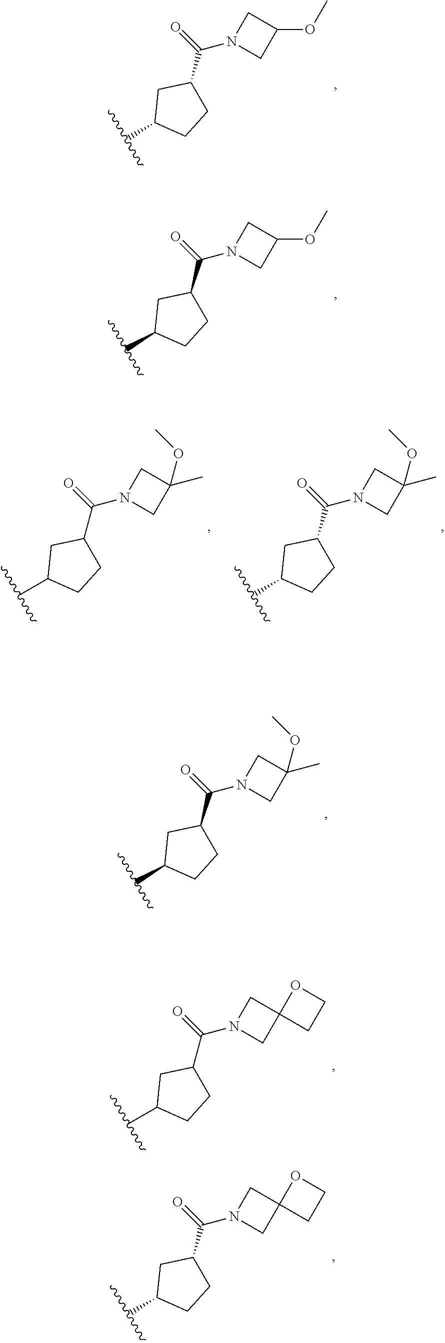

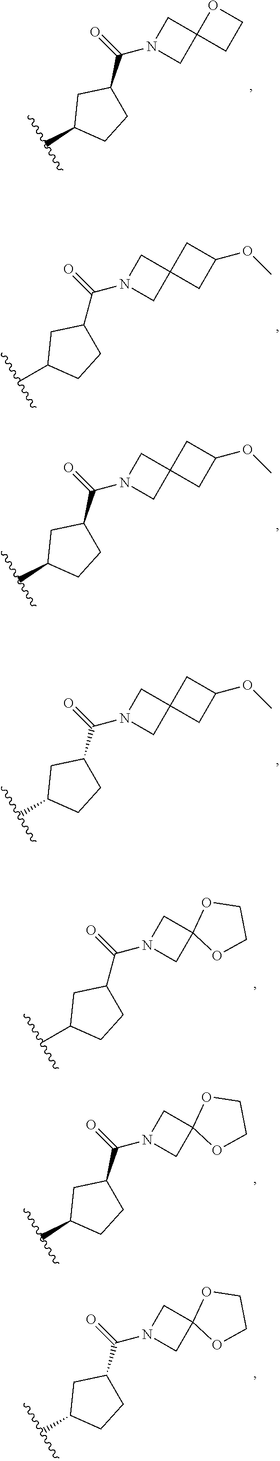

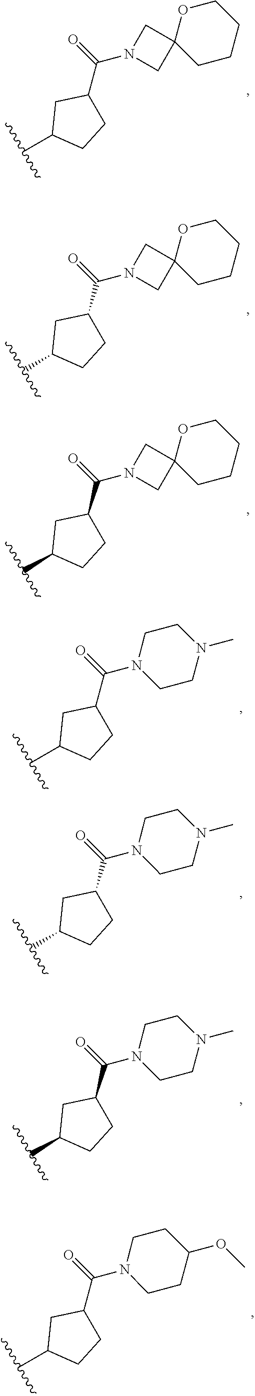

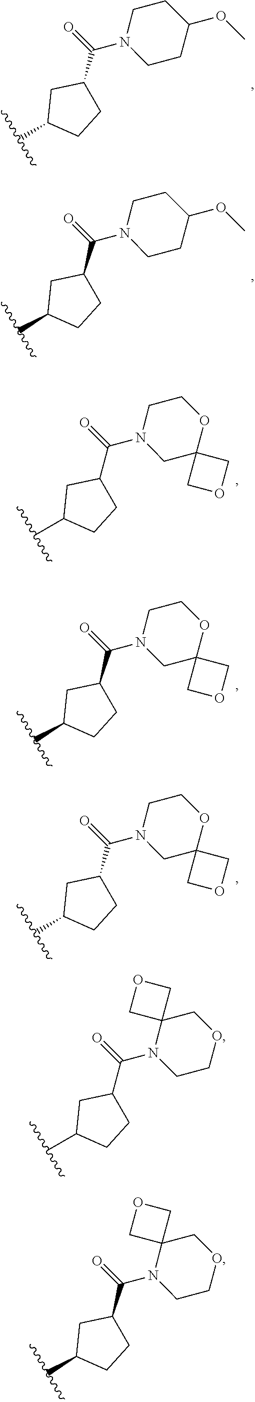

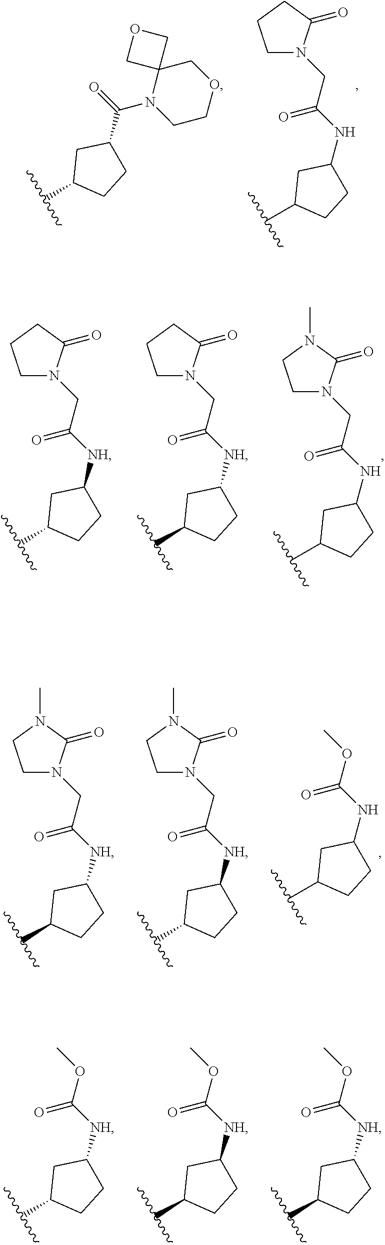

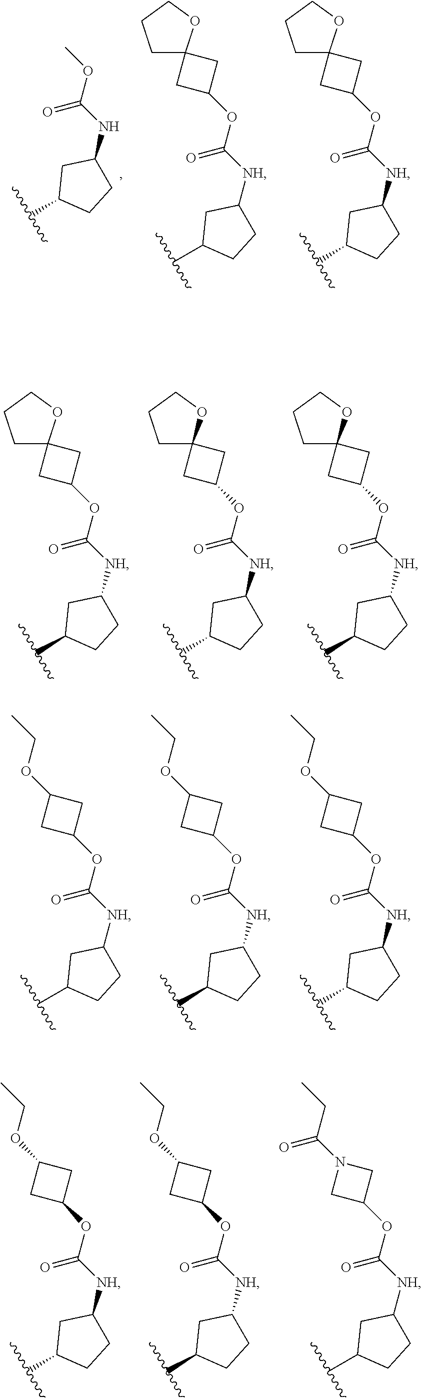

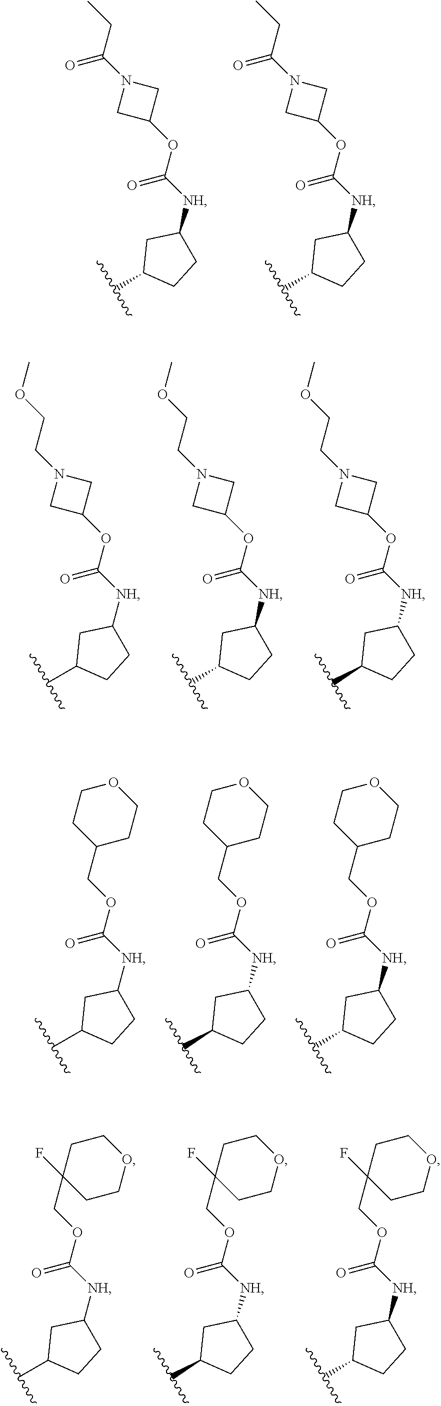

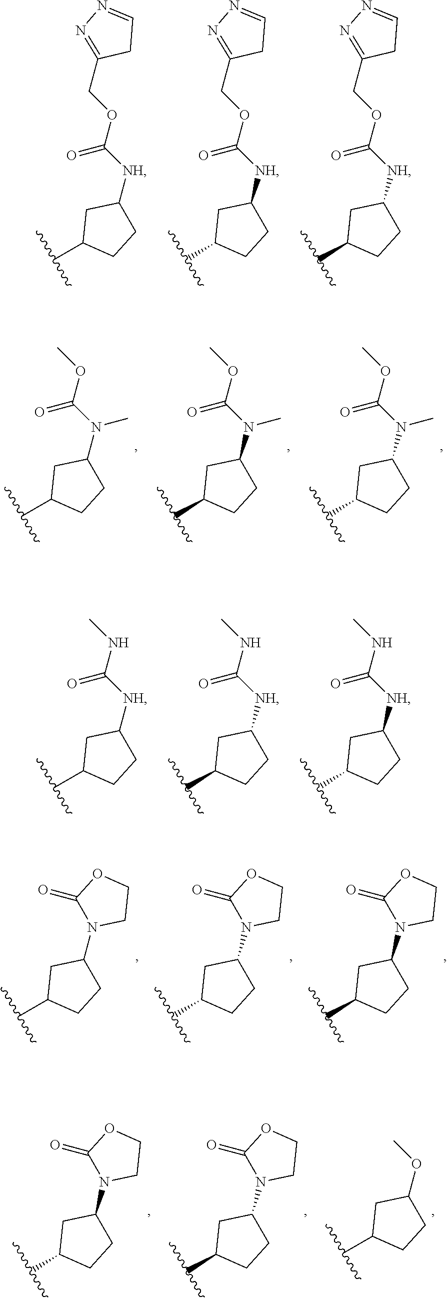

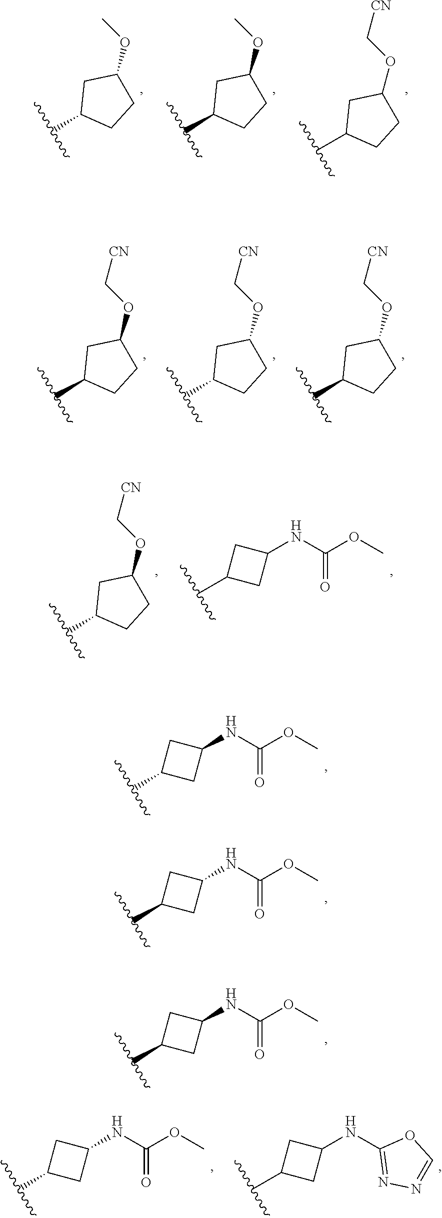

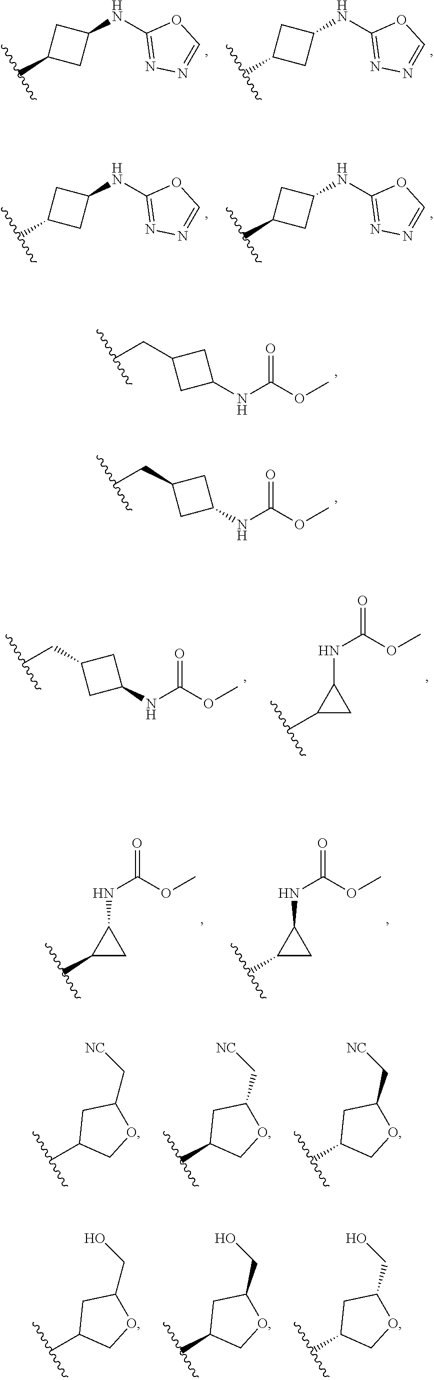

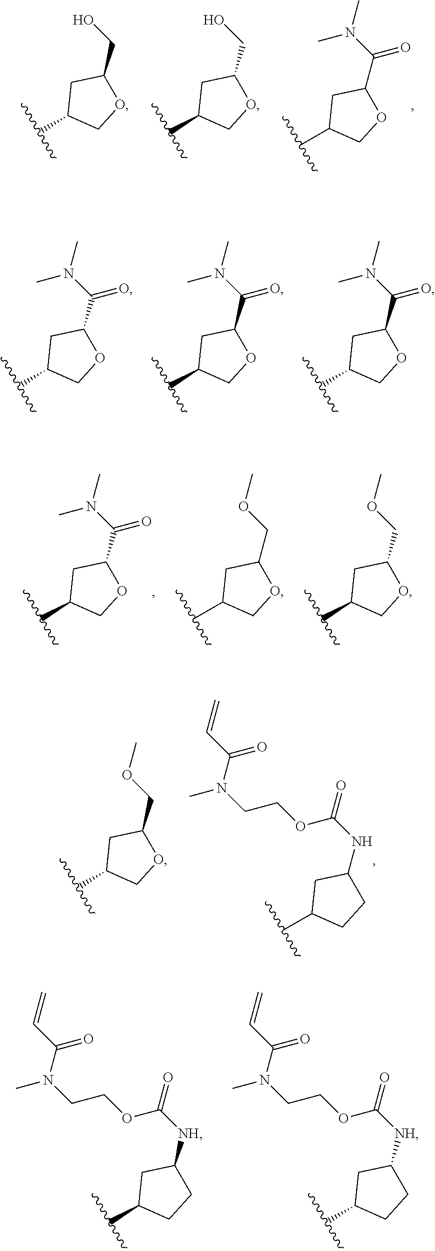

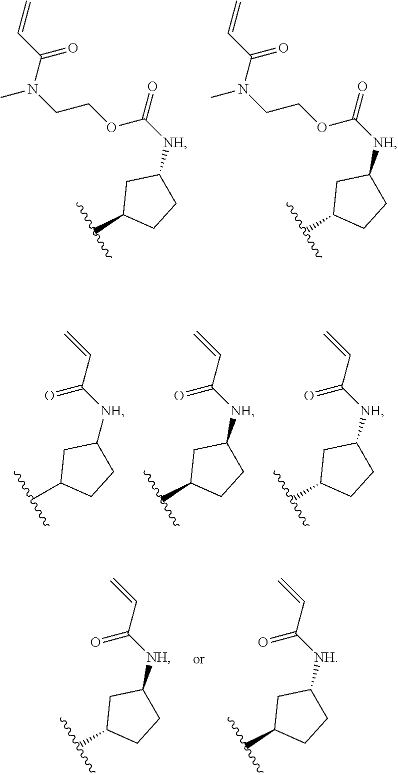

In some embodiments, R² that is substituted with one or more -L-R⁵ or R⁷ is selected from:

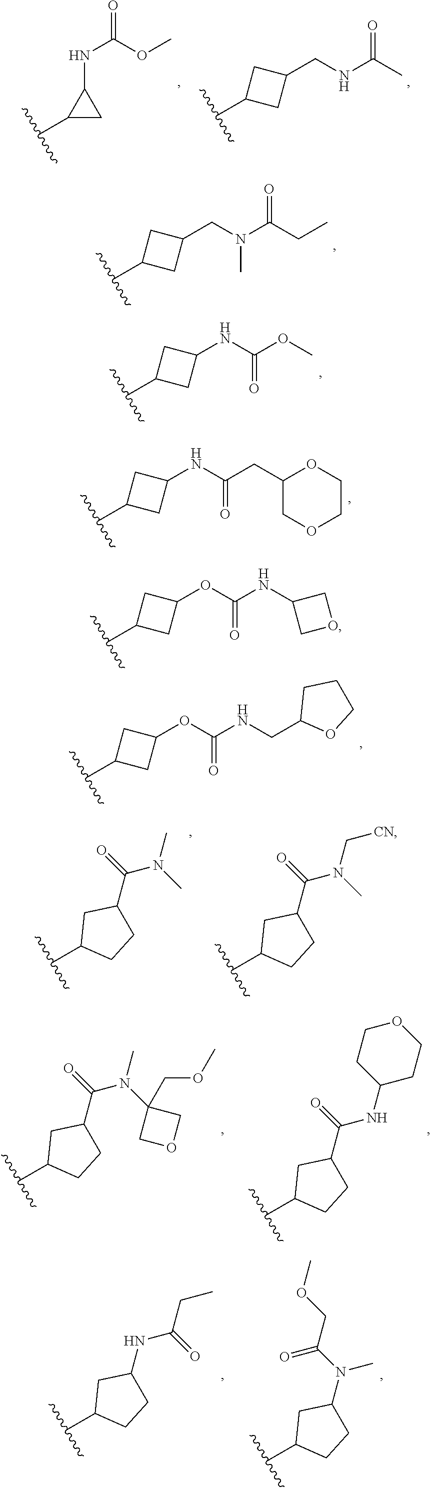

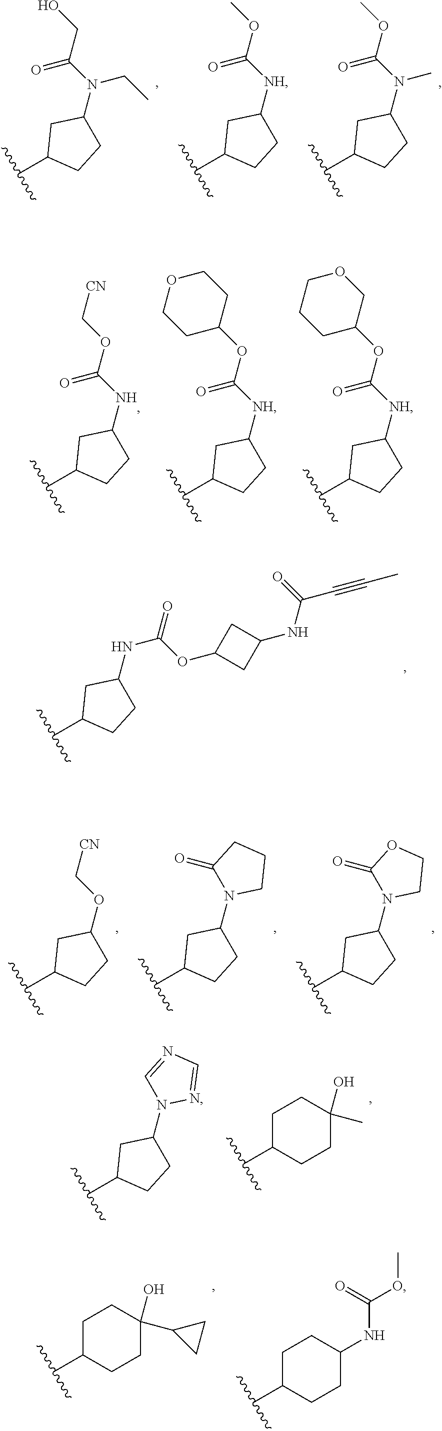

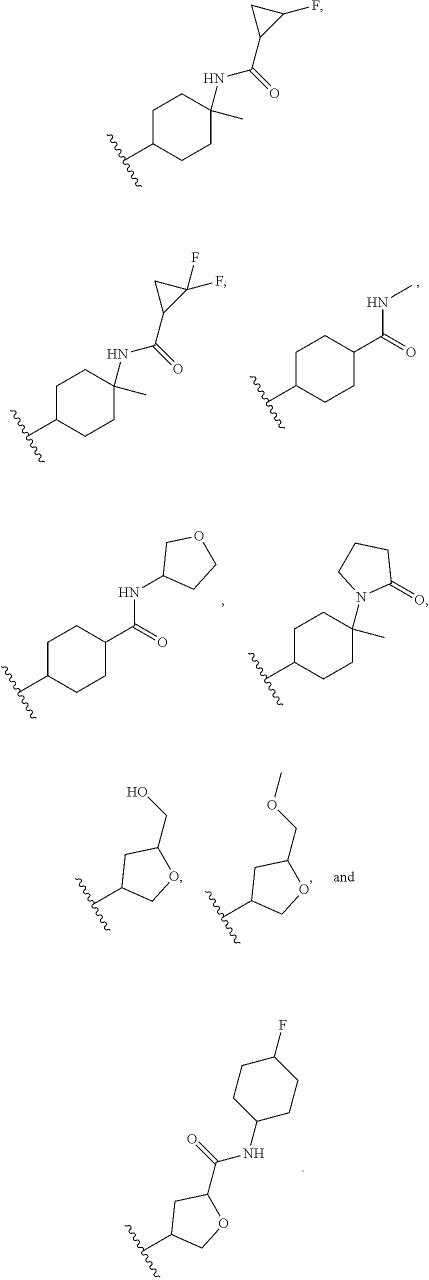

In some embodiments, R² that is substituted with one or more -L-R⁵ or R⁷ is selected from:

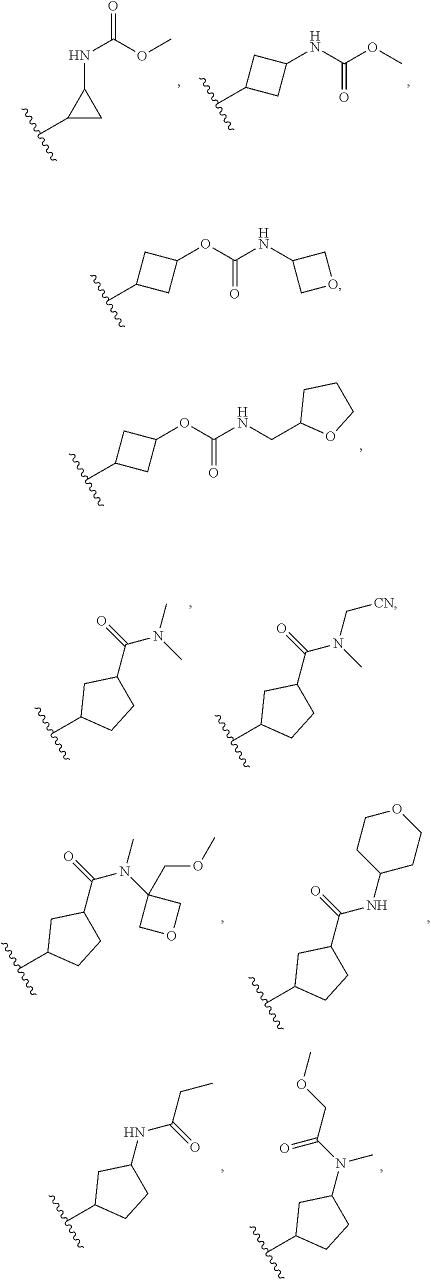

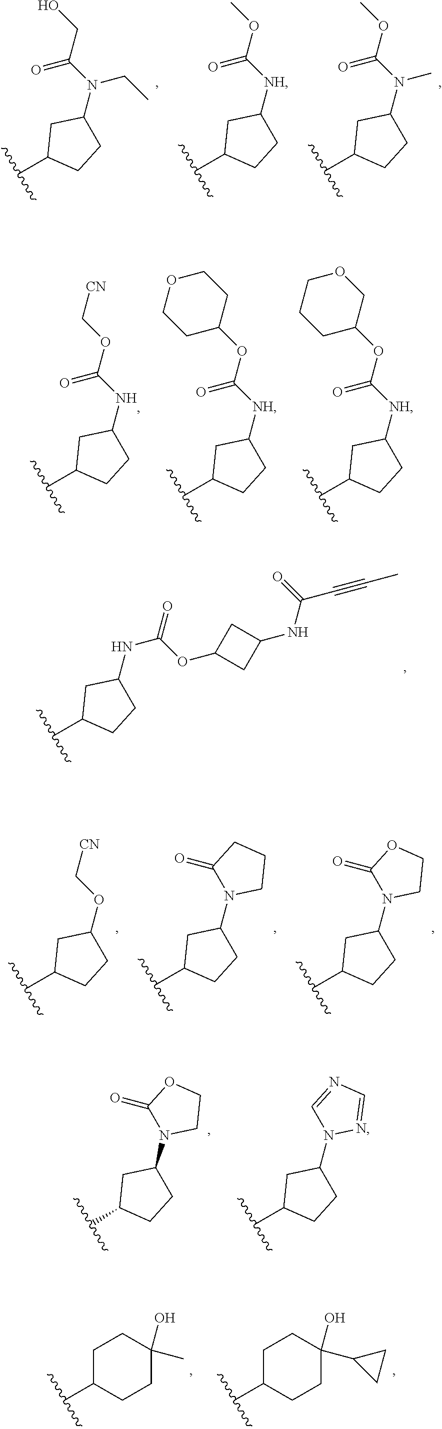

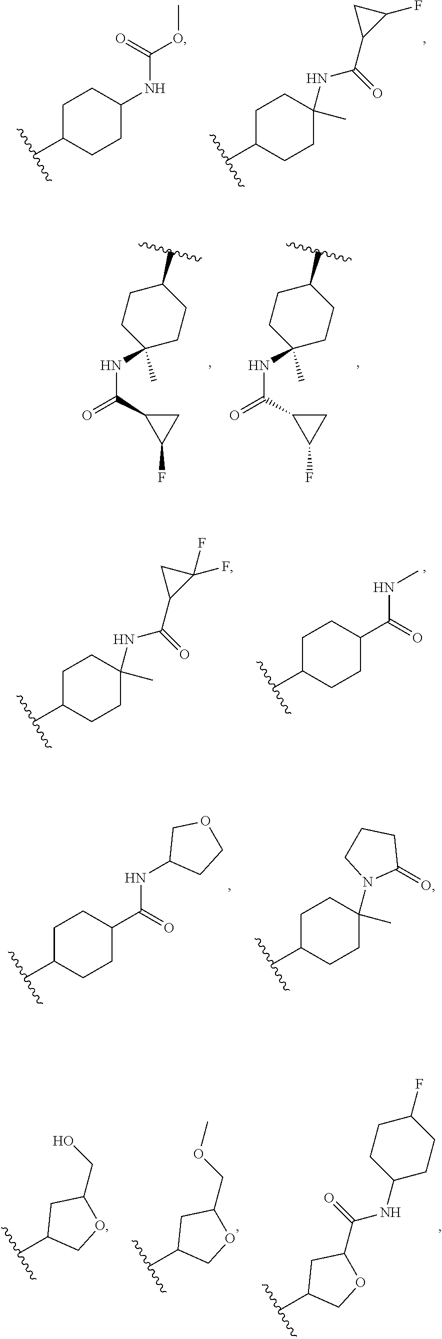

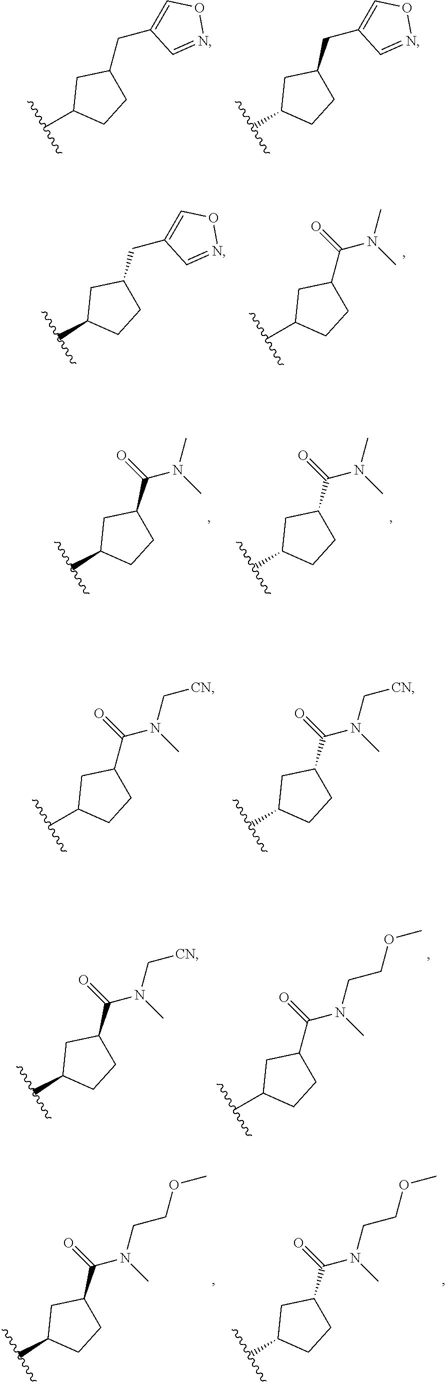

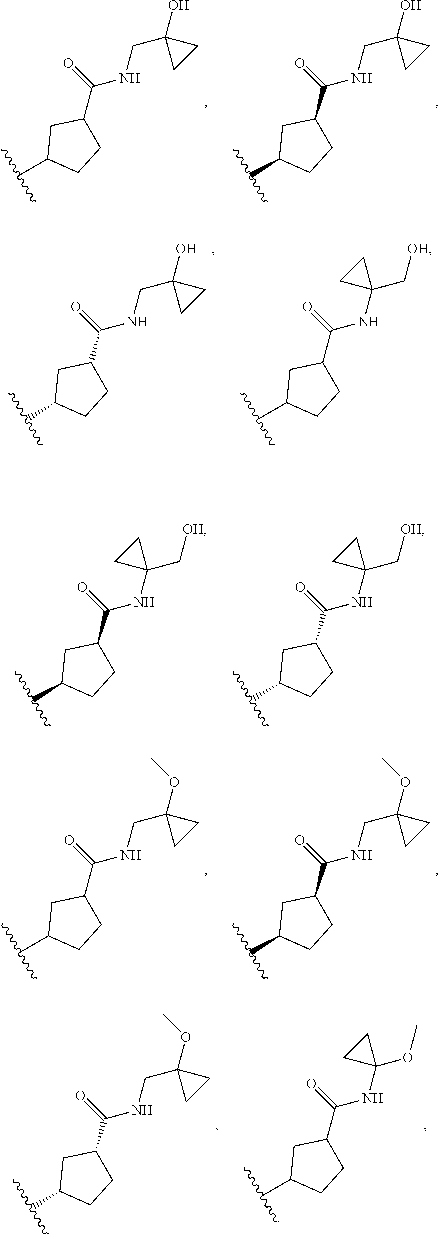

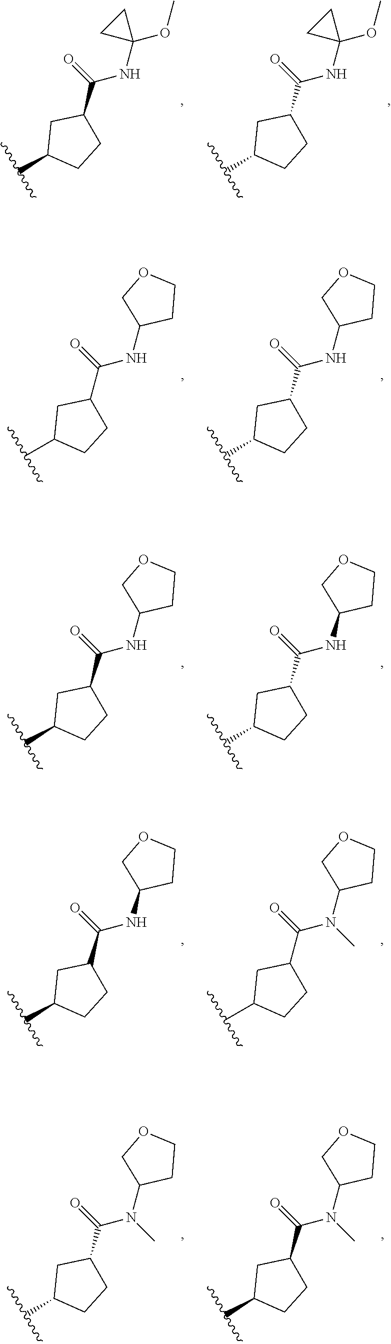

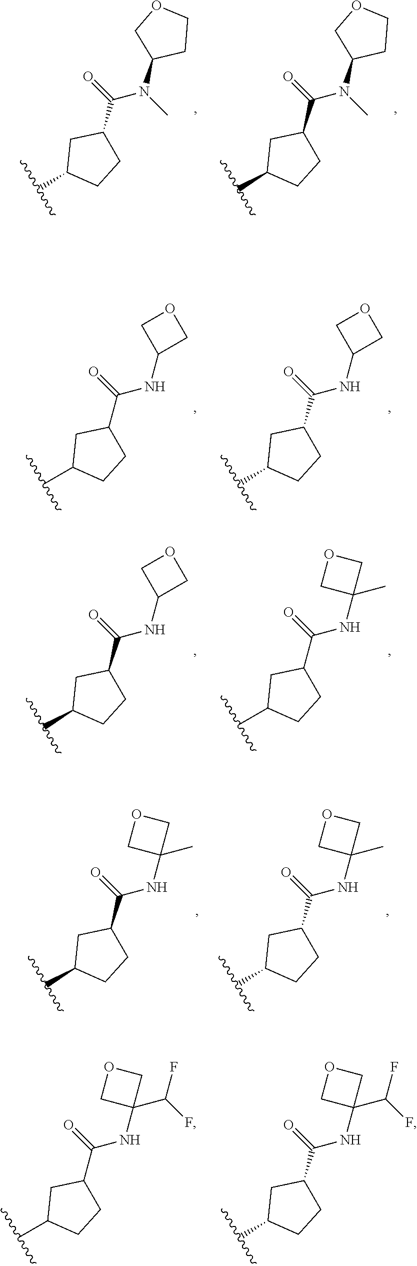

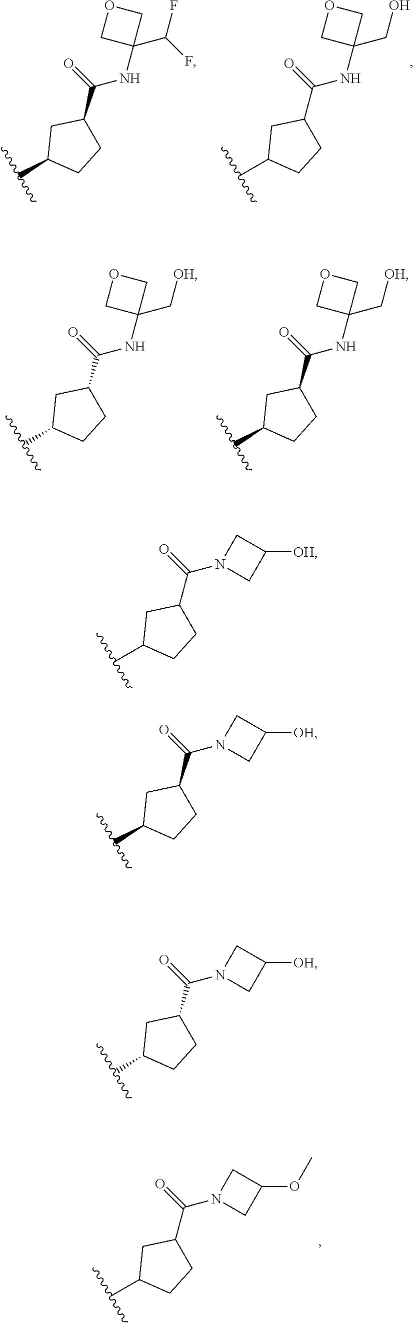

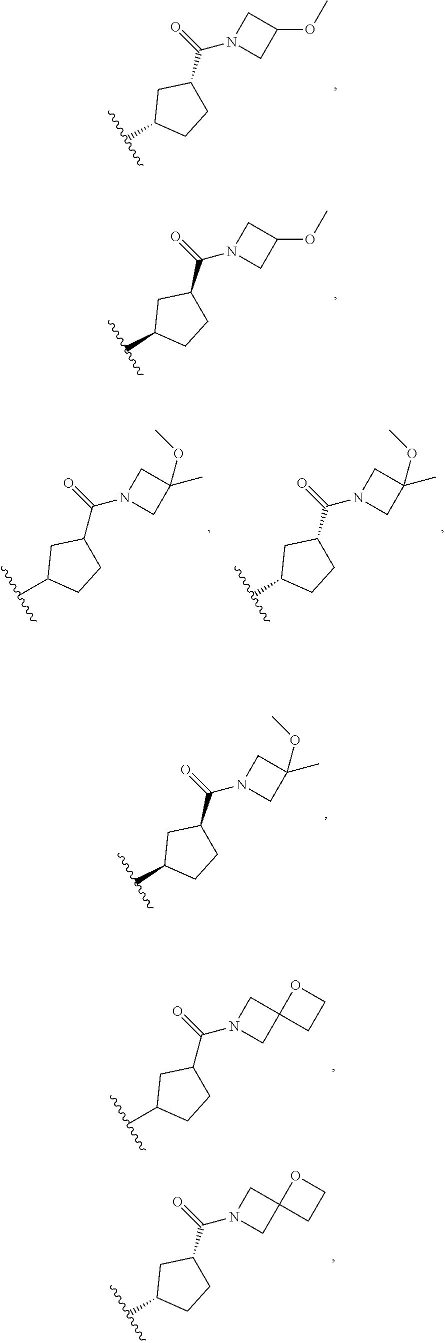

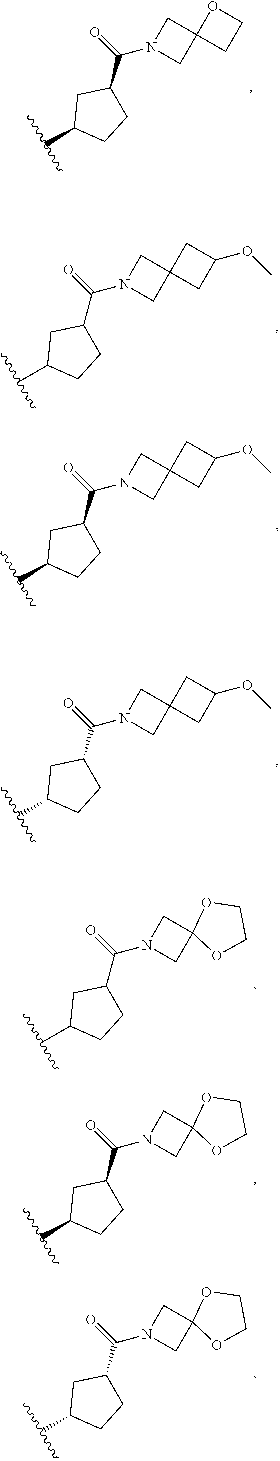

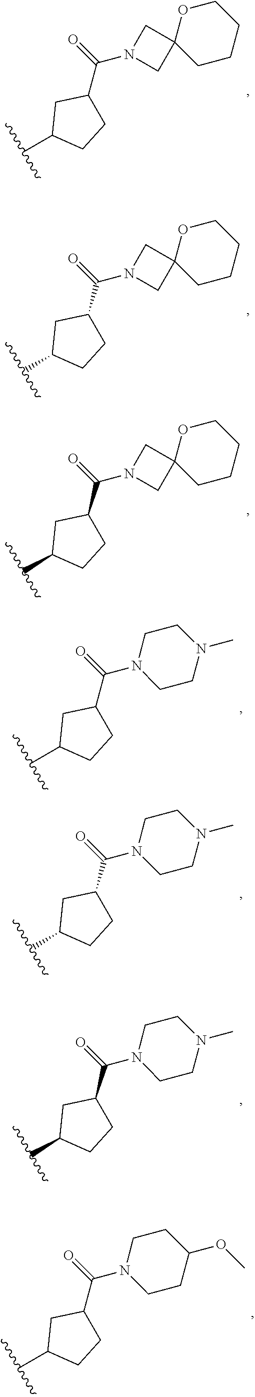

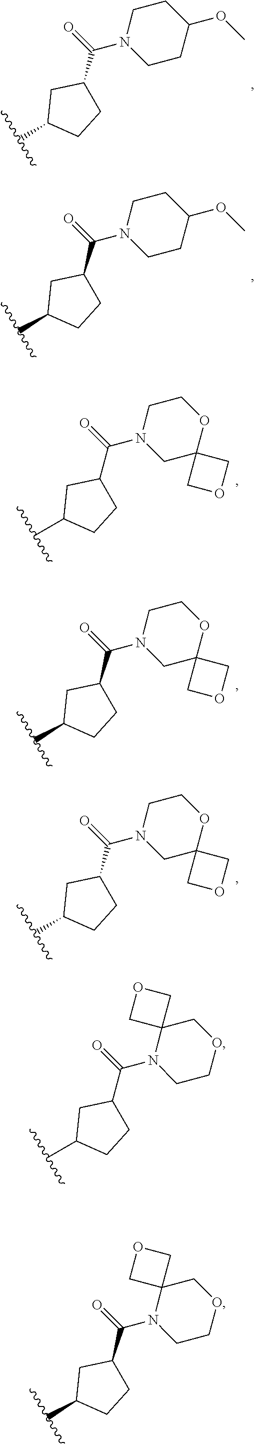

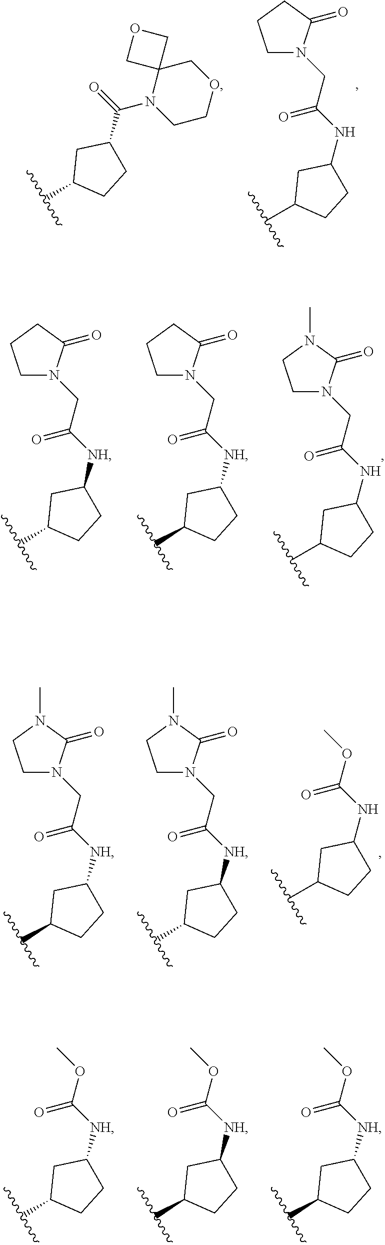

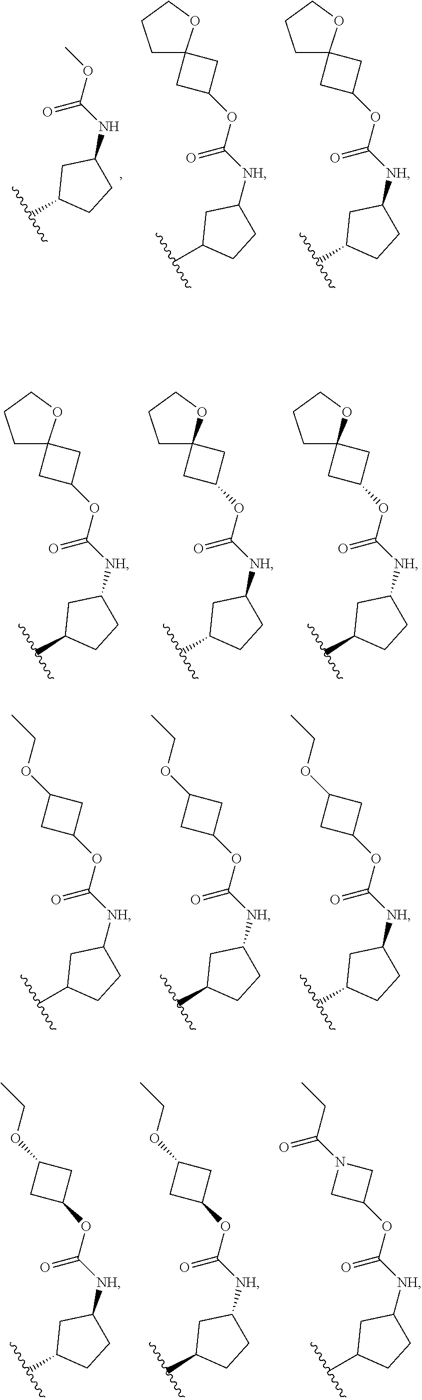

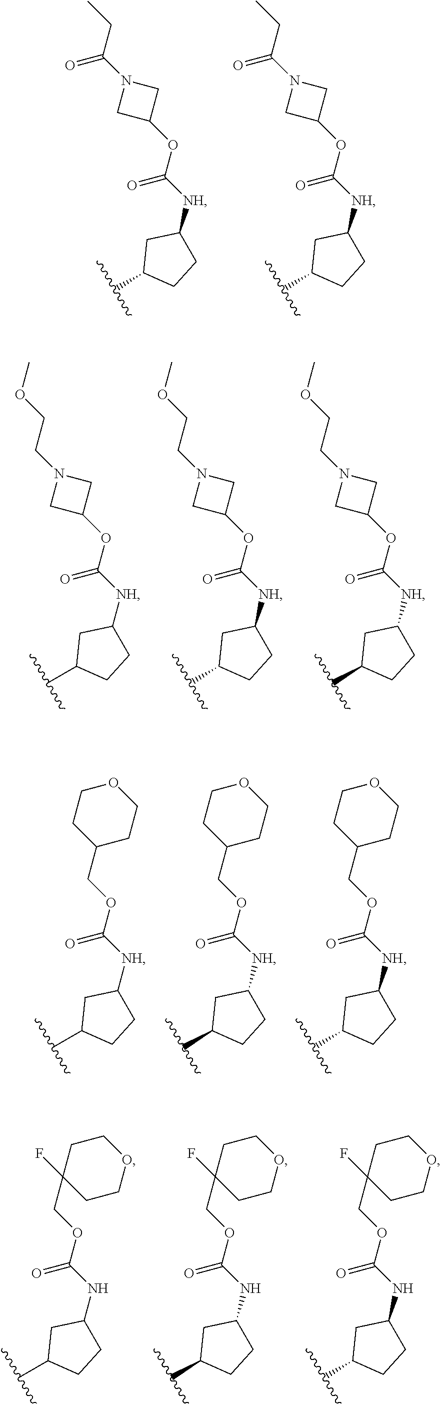

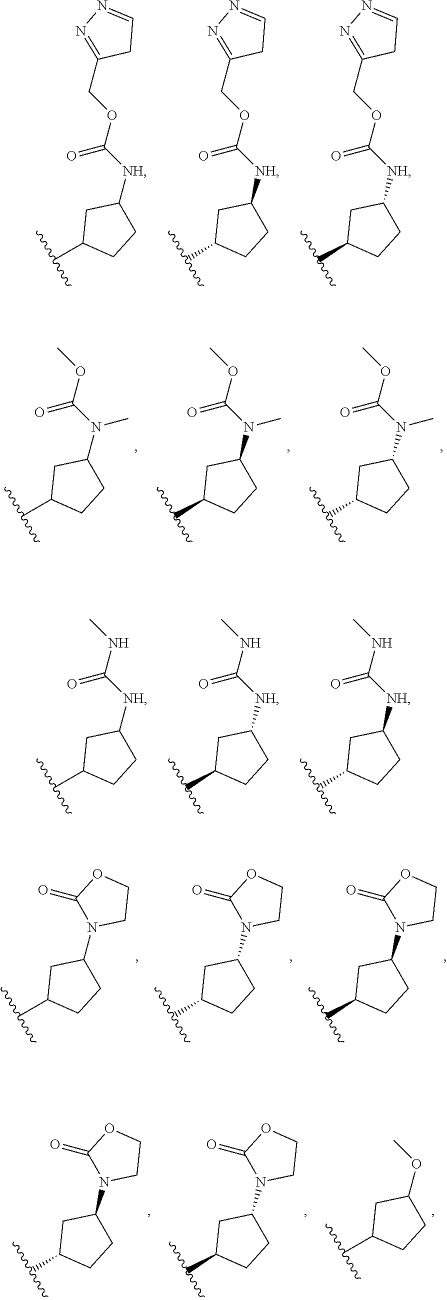

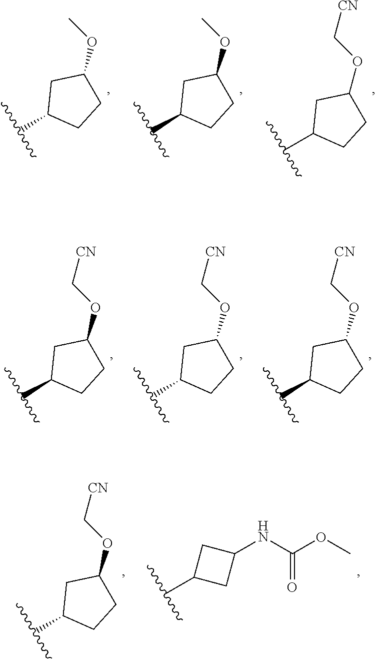

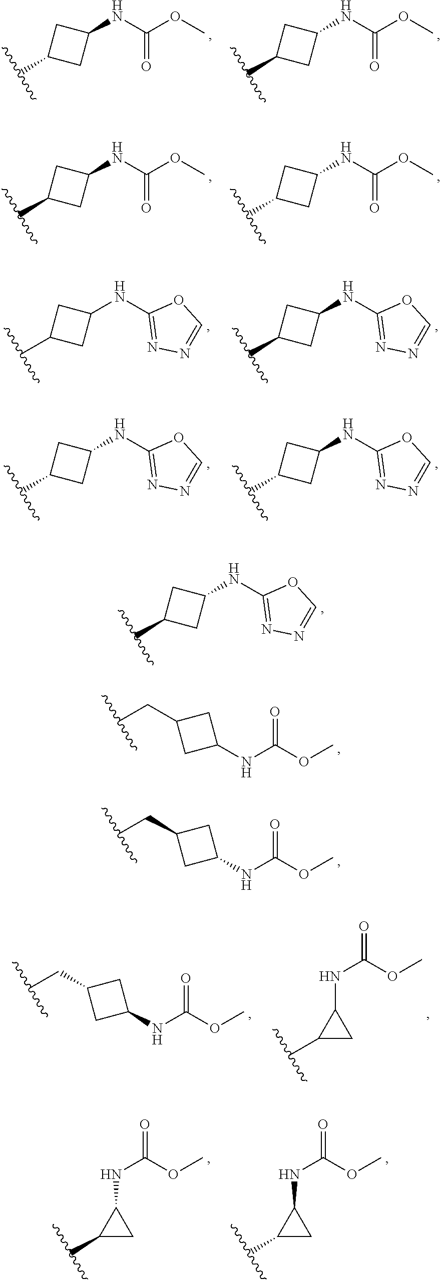

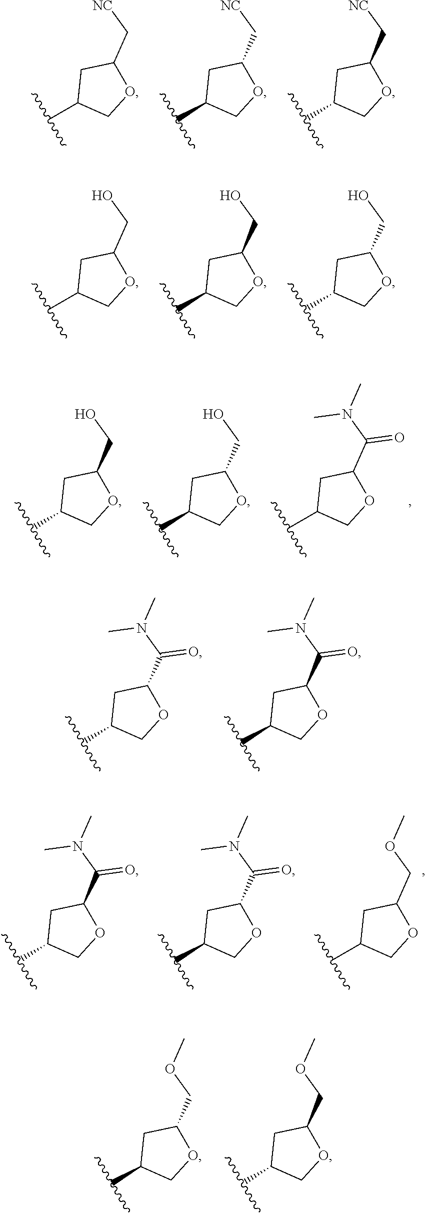

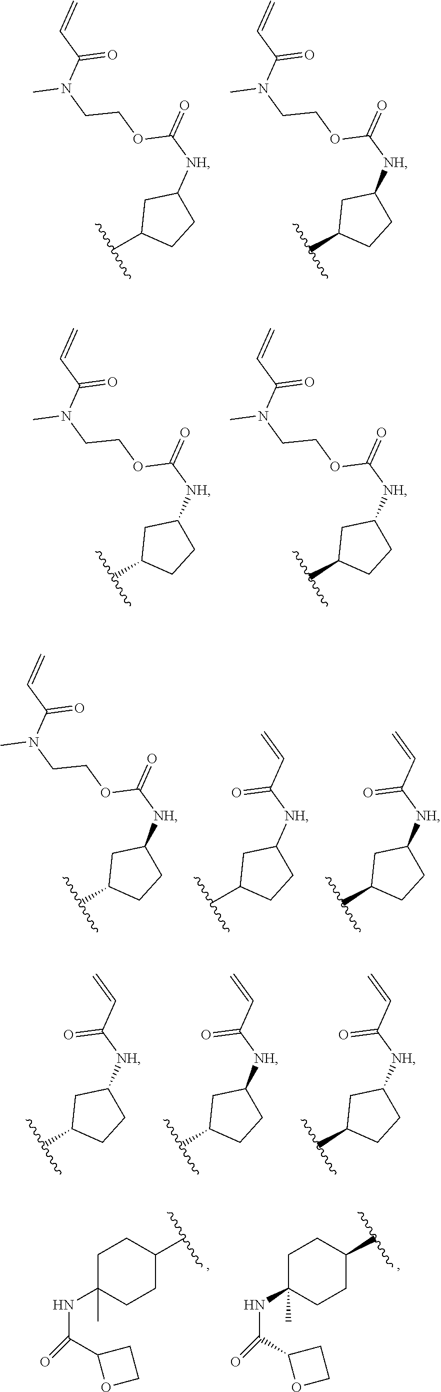

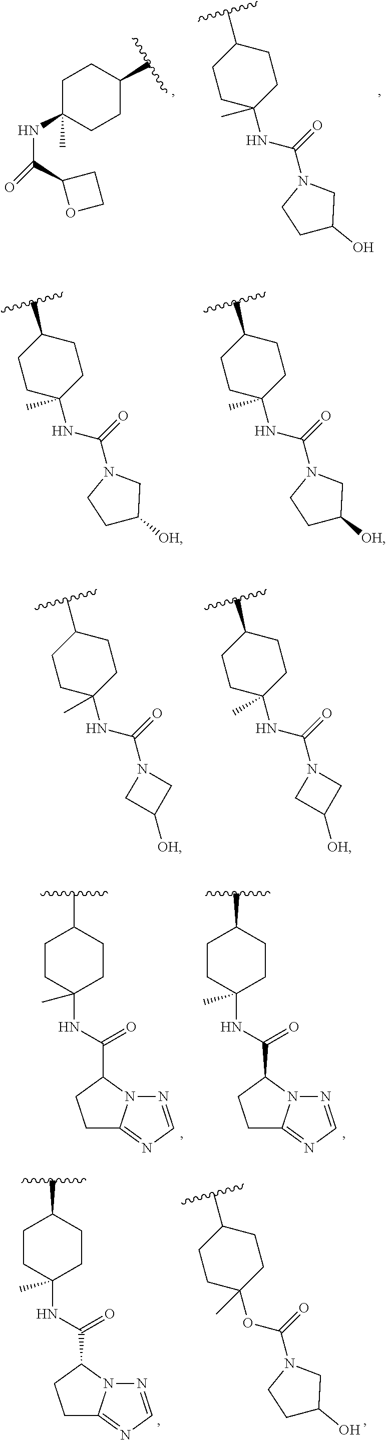

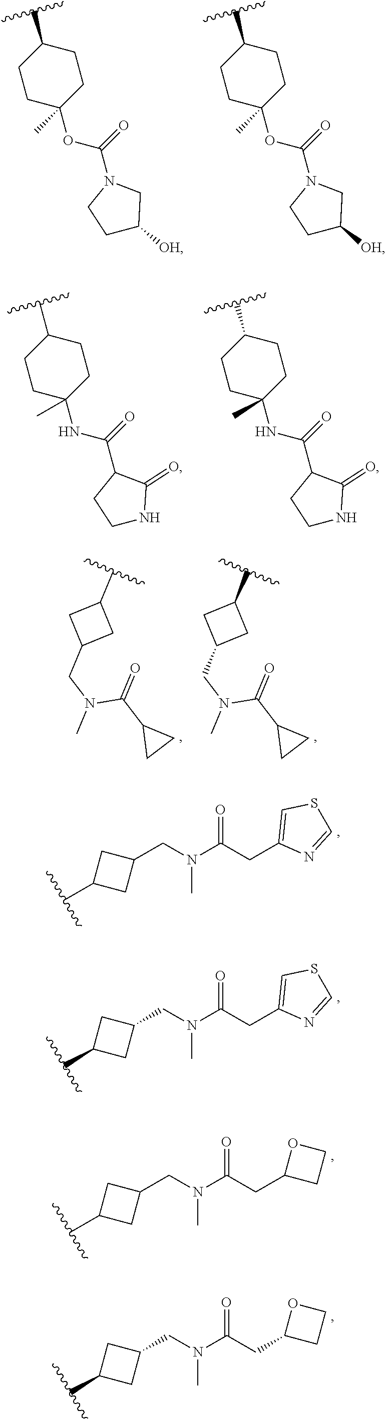

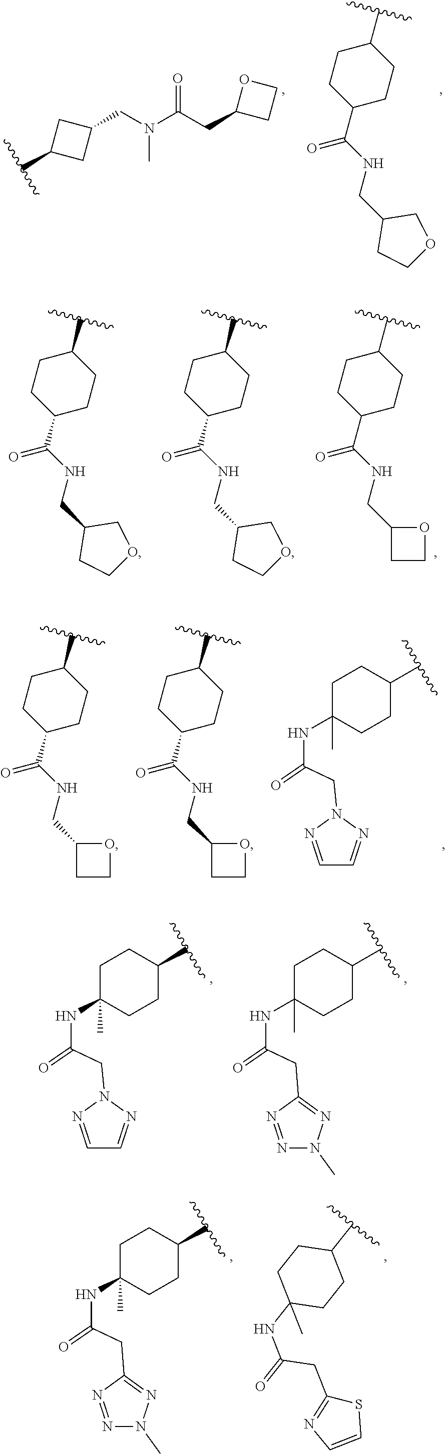

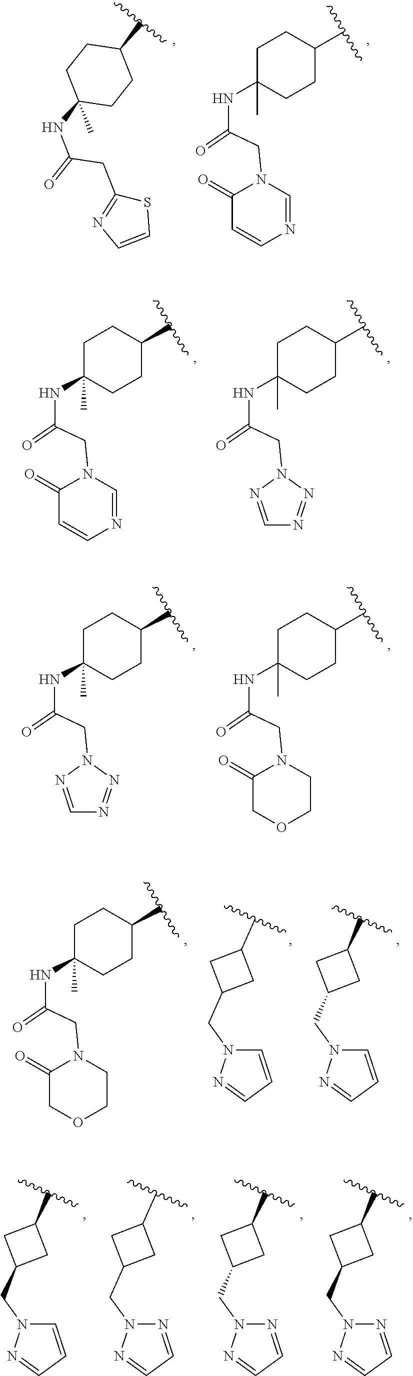

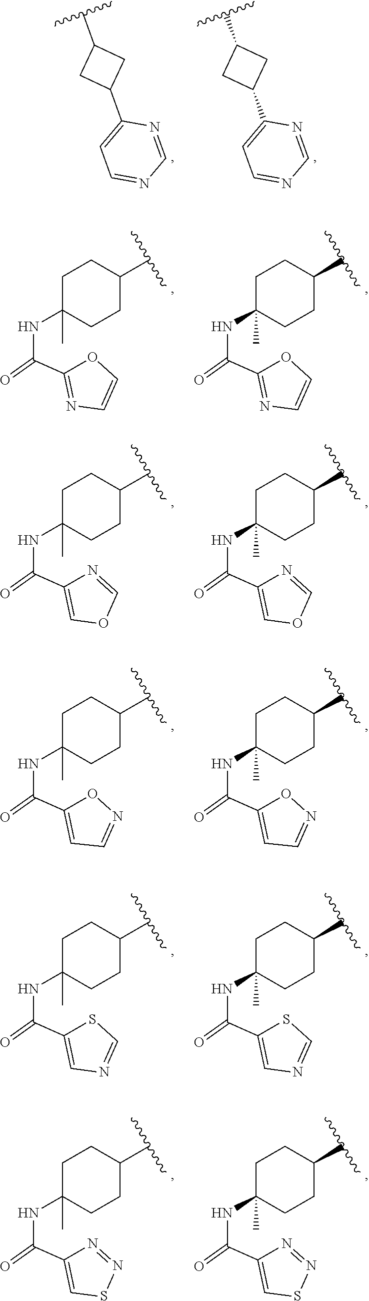

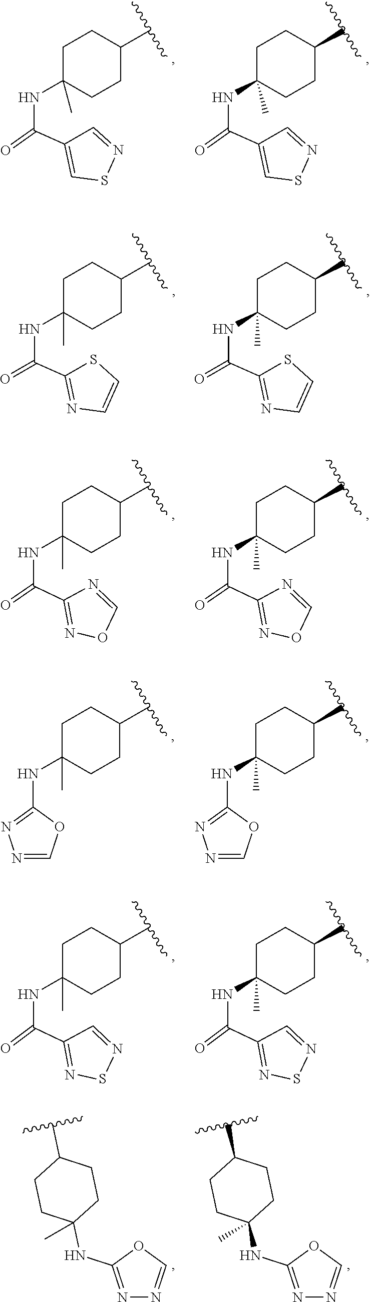

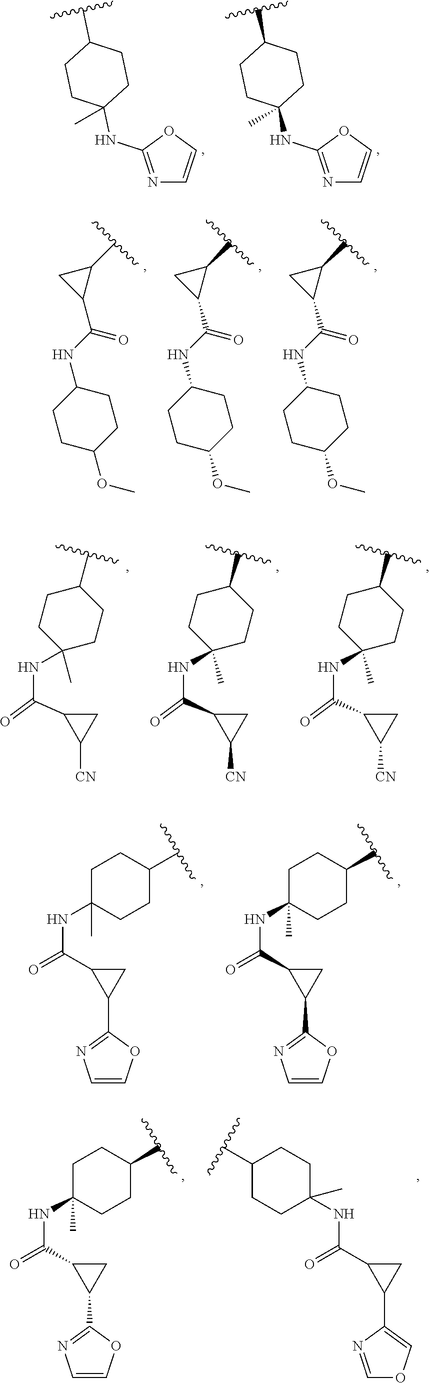

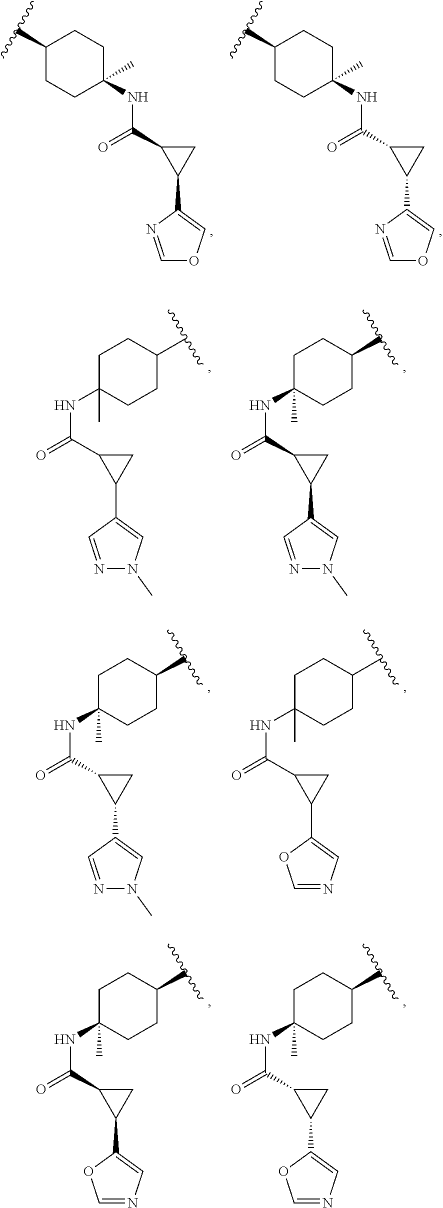

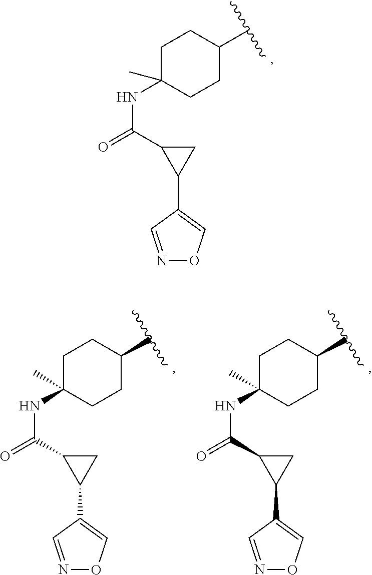 [SMILES: *C1CCC(C)(NC(=O)C2CC2c2cnoc2)CC1 ; *[C@H]1CC[C@@](C)(NC(=O)[C@@H]2C[C@@H]2c2cnoc2)CC1 ; *[C@H]1CC[C@@](C)(NC(=O)[C@H]2C[C@H]2c2cnoc2)CC1]

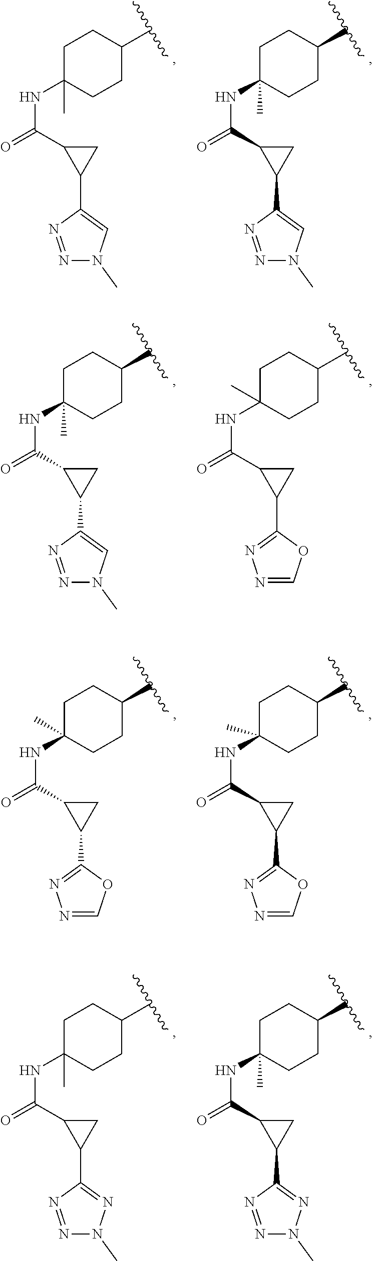

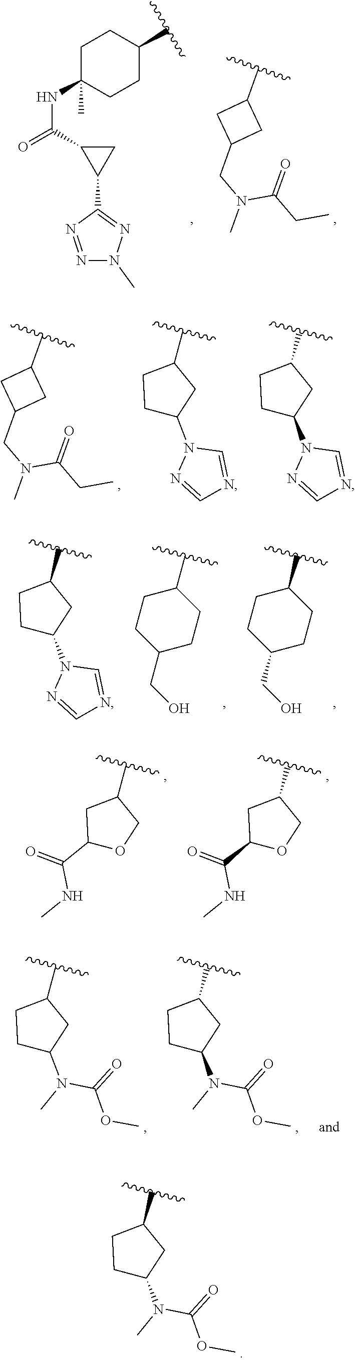

As described herein, with respect to any formula provided herein (e.g., any of Formulae I-IX), R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a}. In some embodiments, R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with 0-3 instances of R^{3a}. In some embodiments, R³ is 5- to 12-membered heteroaryl comprising 1 to 4 heteroatoms selected from N, O, and S, 4- to 12-membered heterocycle comprising 1 to 4 heteroatoms selected from N, O, and S, or C₆-C₁₂ aryl, wherein R³ is optionally substituted with one or more instances (e.g., 0-3 instances) of R^{3a}. In some embodiments, R³ is 5- to 10-membered heteroaryl comprising 1 to 4 heteroatoms selected from N, O, and S, 9- to 10-membered heterocycle comprising 1 to 4 heteroatoms selected from N, O, and S, or phenyl, wherein R³ is optionally substituted with one or more instances (e.g., 0-3 instances) of R^{3a}. In some embodiments, R³ is 5- to 12-membered heteroaryl comprising 1 to 4 heteroatoms selected from N, O, and S or C₆-C₁₂ aryl, wherein R³ is optionally substituted with one or more instances of R^{3a}. In some embodiments, R³ is 5- to 12-membered heteroaryl comprising 1 to 4 heteroatoms selected from N, O, and S or C₆-C₁₂ aryl, wherein R³ is optionally substituted with 0-3 instances of R^{3a}.

In some embodiments, R³ is 5- to 12-membered heteroaryl comprising 1 to 4 heteroatoms selected from N, O, and S, and wherein R³ is optionally substituted with one or more instances of R^{3a}. In some embodiments, R³ is 5- to 6-membered heteroaryl comprising 1 to 3 heteroatoms selected from N, O, and S, optionally substituted with one or more instances of R^{3a}. In some embodiments, R³ is a 5-membered heteroaryl comprising 1 to 4 heteroatoms selected from N, O, and S, optionally substituted with one or more instances of R^{3a}. In some embodiments, R³ is a pyrazole optionally substituted with one or more instances of R^{3a}. In some embodiments, R³ is a 6-membered heteroaryl comprising 1 to 4 heteroatoms selected from N, O, and S, optionally substituted with one or more instances of R^{3a}. In some embodiments, R³ is a pyridine or pyridazone optionally substituted with one or more instances of R^{3a}.

In some embodiments, R³ is bicyclic 7- to 12-membered heteroaryl comprising 1 to 4 heteroatoms selected from N, O, and S, and wherein R³ is optionally substituted with one or more instances of R^{3a}. In some embodiments, R³ is bicyclic 8- to 10-membered heteroaryl comprising 1 to 4 heteroatoms selected from N, O, and S, and wherein R³ is optionally substituted with one or more instances of R^{3a}. In some embodiments, R³ is bicyclic 9-membered heteroaryl comprising 1 to 3 heteroatoms selected from N, O, and S, and wherein R³ is optionally substituted with one or more instances of R^{3a}. In some embodiments, R³ is an indazole, thienopyridine, benzoxazole, pyrazolopyridine, or imidazopyridine, optionally substituted with one or more instances of R^{3a}. In some embodiments, R³ is indole, indazole, thienopyridine, benzothiophene, or pyrrolopyridine, optionally substituted with one or more instances of R^{3a}. In some embodiments, R³ is bicyclic 7- to 12-membered heteroaryl comprising 1 to 4 heteroatoms selected from N, O, and S, optionally substituted with one or more instances of R^{3a}, and wherein R^{3a} is optionally substituted C₁-C₆ aliphatic, optionally substituted C₃-C₁₂ cycloaliphatic, or optionally substituted 4- to 6-membered heterocycle comprising 1 to 4 heteroatoms selected from N, O, and S.

In some embodiments, R³ is

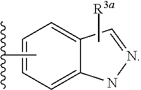

In some embodiments R³ is

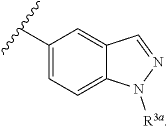

In some embodiments, R³ is:

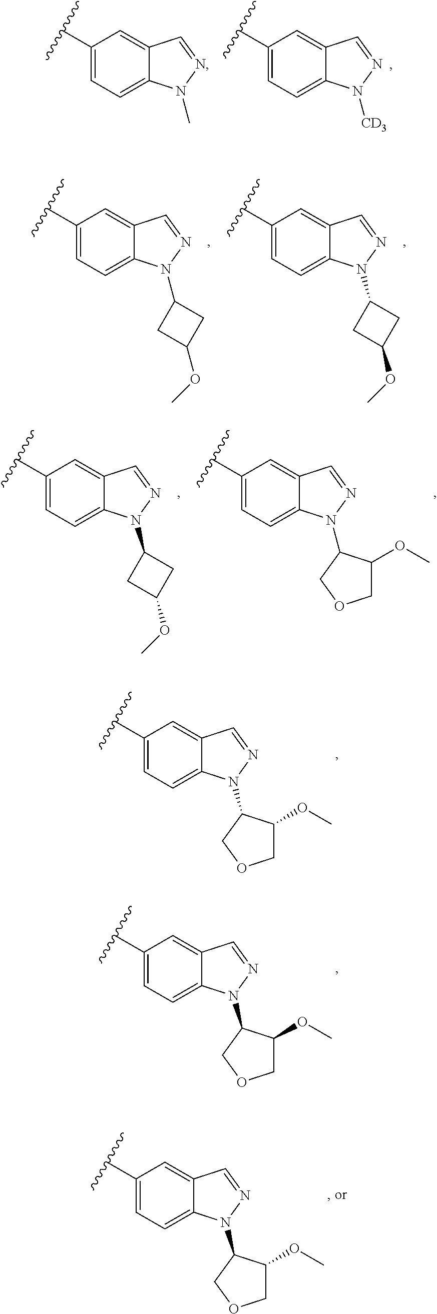

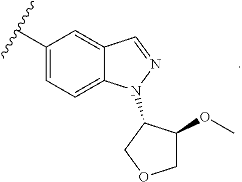 [SMILES: *c1ccc2c(cnn2[C@H]2COC[C@@H]2OC)c1]

In some embodiments, R³ is

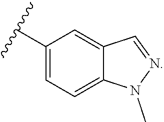 [SMILES: *c1ccc2c(cnn2C)c1]

In some embodiments, R³ is 4- to 12-membered heterocycle comprising 1 to 4 heteroatoms selected from N, O, and S, wherein R³ is optionally substituted with one or more instances of R^{3a}. In some embodiments, R³ is an optionally substituted 6- to 10-membered heterocycle comprising 1 to 4 heteroatoms selected from N, O, and S. In some embodiments, R³ is bicyclic 6- to 12-membered heterocycle comprising 1 to 4 heteroatoms selected from N, O, and S, and wherein R³ is optionally substituted with one or more instances of R^{3a}. In some embodiments, R³ is bicyclic 6- to 12-membered heterocycle comprising 1 to 4 heteroatoms selected from N, O, and S, and wherein R³ is optionally substituted with one or more instances of R^{3a}, and wherein R^{3a} is optionally substituted C₁-C₆ aliphatic. In some embodiments, R³ is bicyclic 6- to 12-membered heterocycle comprising 1 to 4 heteroatoms selected from N, O, and S, and wherein R³ is optionally substituted with one or more instances of R^{3a}, and wherein each R^{3a} is independently C₁-C₆ aliphatic or oxo. In some embodiments, R³ is bicyclic 6- to 12-membered heterocycle comprising 1 to 4 heteroatoms selected from N, O, and S, and wherein R³ is optionally substituted with one or more instances of R^{3a}, and wherein R^{3a} is —CH₃. In some embodiments, R³ is bicyclic 6- to 12-membered heterocycle comprising 1 to 4 heteroatoms selected from N, O, and S, and wherein R³ is optionally substituted with one or more instances of R^{3a}, and wherein each R^{3a} is independently —CH₃ or oxo. In some embodiments, R³ is a bicyclic 9- to 10-membered heterocycle comprising 1 to 3 heteroatoms selected from N, O, and S, and wherein R³ is optionally substituted with one or more instances of R^{3a}.

In some embodiments, R³ is C₆-C₁₂ aryl, and wherein R³ is optionally substituted with one or more instances of R^{3a}. In some embodiments, R³ is phenyl optionally substituted with one or more instances or R^{3a}. In some embodiments, R³ is phenyl optionally substituted with one or more instances of R^{3a}, and wherein R^{3a} is optionally substituted C₁-C₆ aliphatic, optionally substituted 2- to 10-membered heteroaliphatic, or optionally substituted 4- to 10-membered heterocycle comprising 1 to 4 heteroatoms selected from N, O, and S. In some embodiments, R³ is

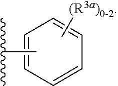

In some embodiments, R³ is

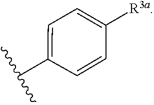

In some embodiments, R³ is

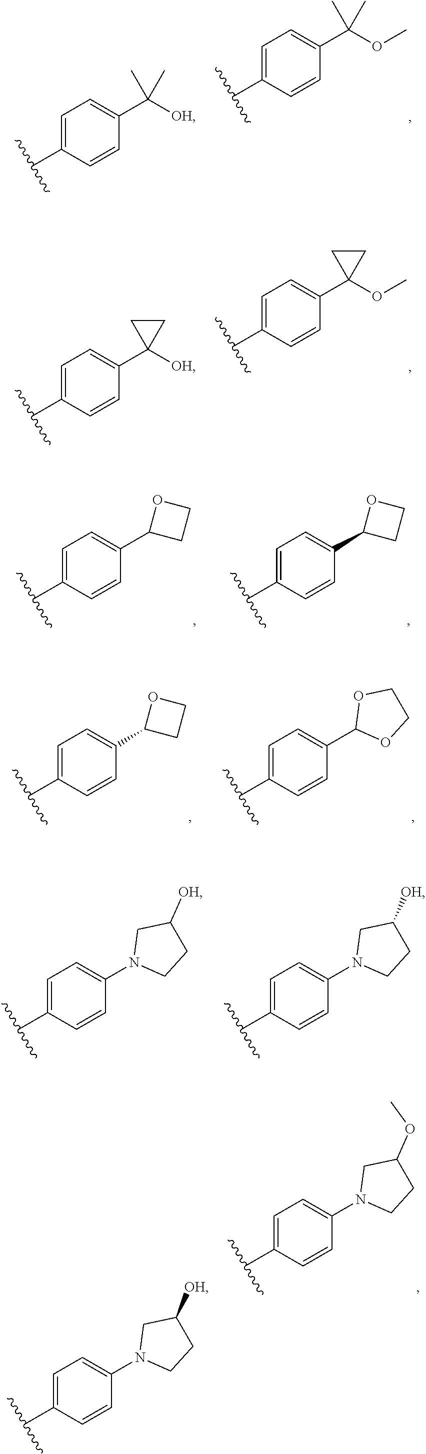

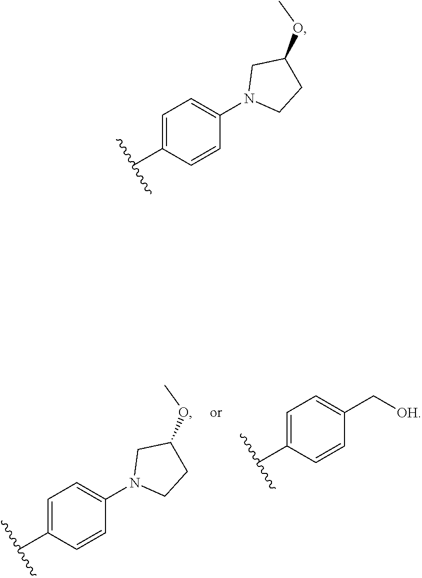 [SMILES: *c1ccc(N2CC[C@H](OC)C2)cc1 ; *c1ccc(N2CC[C@@H](OC)C2)cc1 ; *c1ccc(CO)cc1]

In some embodiments, R³ is C₃-C₁₂ cycloaliphatic, and wherein R³ is optionally substituted with one or more instances of R^{3a}. In some embodiments, R³ is a C₃-C₇ monocyclic cycloaliphatic, and wherein R³ is optionally substituted with one or more instances of R^{3a}. In some embodiments, R³ is a C₃-C₇ monocyclic cycloalkyl, and wherein R³ is optionally substituted with one or more instances of R^{3a}. In some embodiments, R³ is a C₅-C₁₂ bicyclic cycloaliphatic, and wherein R³ is optionally substituted with one or more instances of R^{3a}. In some embodiments, R³ is a C₅-C₁₂ bicyclic cycloalkyl, and wherein R³ is optionally substituted with one or more instances of R^{3a}.

In some embodiments, R³ is C₁-C₆ aliphatic, wherein R³ is optionally substituted with one or more instances of R^{3a}. In some embodiments, R³ is C₁-C₆ alkyl, wherein R³ is optionally substituted with one or more instances of R^{3a}.

In some embodiments, R³ is substituted with 0 to 4 instances of R^{3a}. In some embodiments, R³ is substituted with 0 to 3 instances of R^{3a}. In some embodiments, R³ is substituted with 0 to 2 instances of R^{3a}. In some embodiments, R³ is unsubstituted. In some embodiments, R³ is substituted with 1 to 3 instances of R^{3a}. In some embodiments, R³ is substituted with 1 to 2 instances of R^{3a}. In some embodiments, R³ is substituted with 1 instance of R^{3a}. In some embodiments, R³ is substituted with 2 instances of R^{3a}. In some embodiments, R³ is substituted with 3 instances of R^{3a}. In some embodiments, R³ is substituted with 4 instances of R^{3a}.

In some embodiments, R³ is selected from:

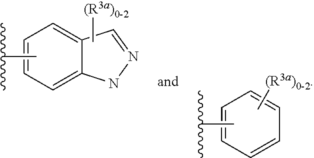

In some embodiments, R³ is selected from:

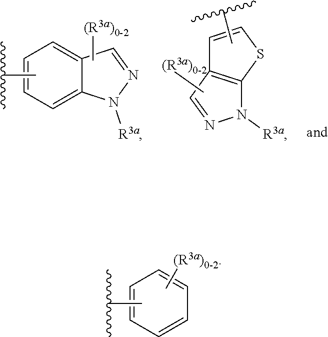

In some embodiments, R³ is selected from

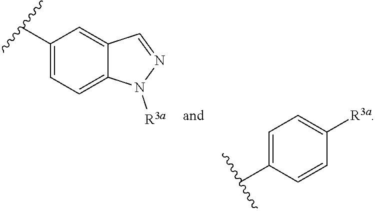

In some embodiments, R³ is selected from

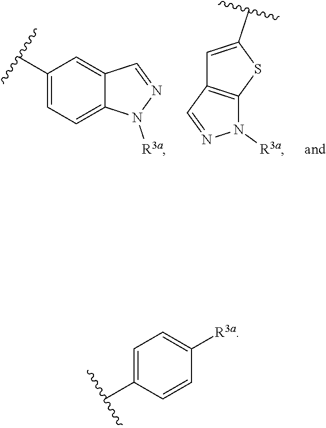

In some embodiments, R³ is selected from

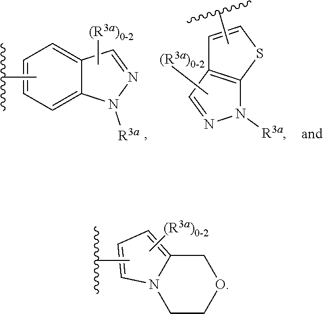

In some embodiments, R³ is selected from

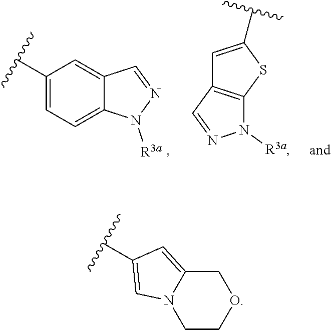

In some embodiments, R³ is selected from

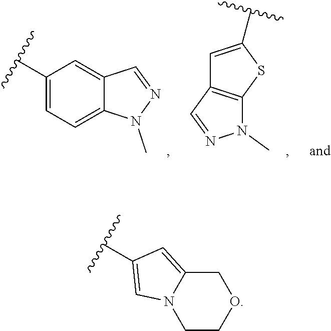

In some embodiments, R³ is:

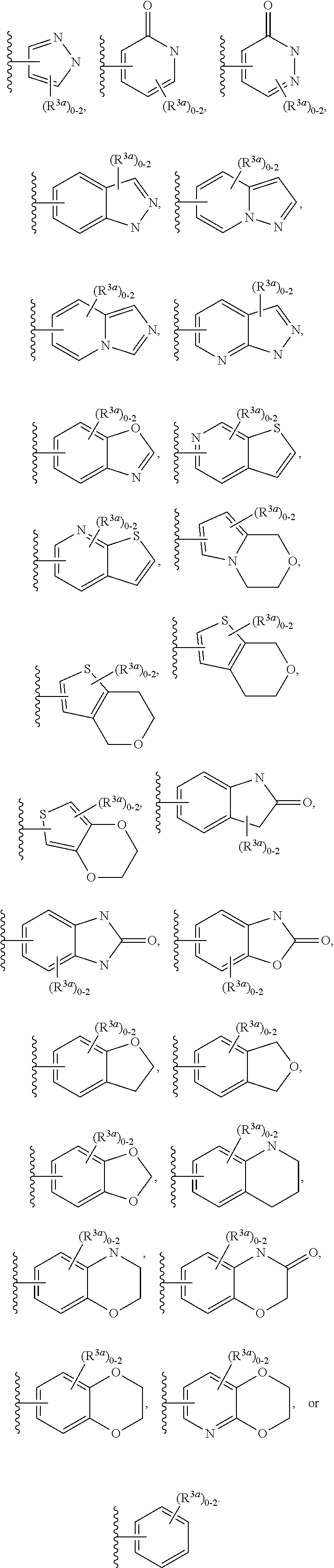

In some embodiments, R³ is:

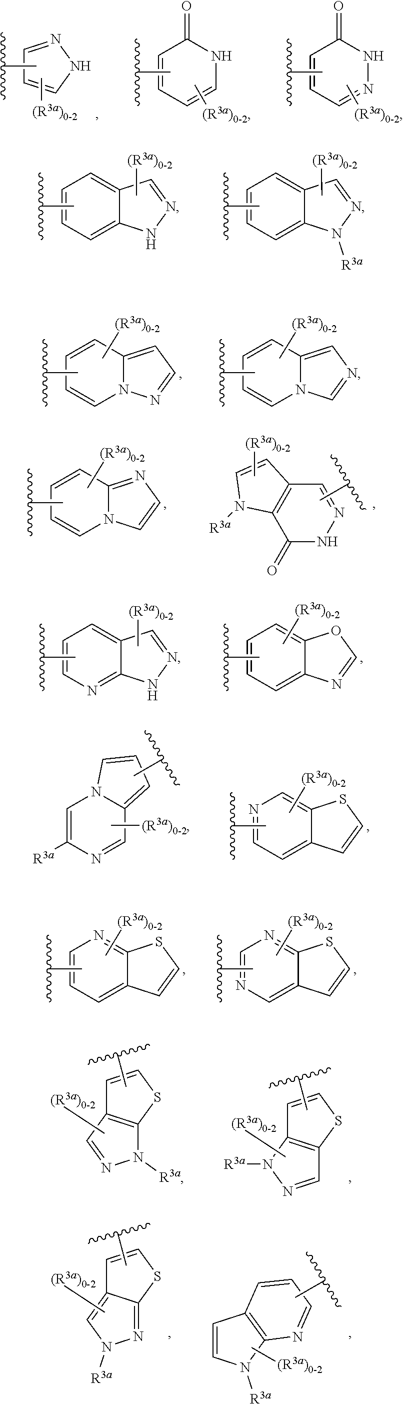

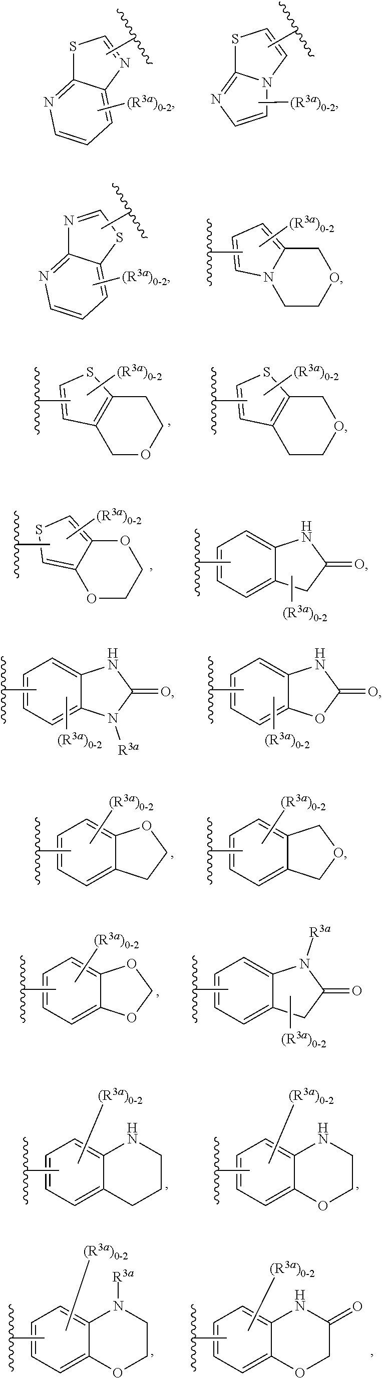

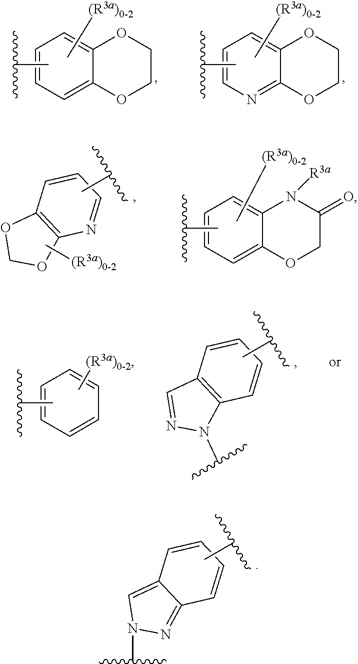

In some embodiments, R³ is:

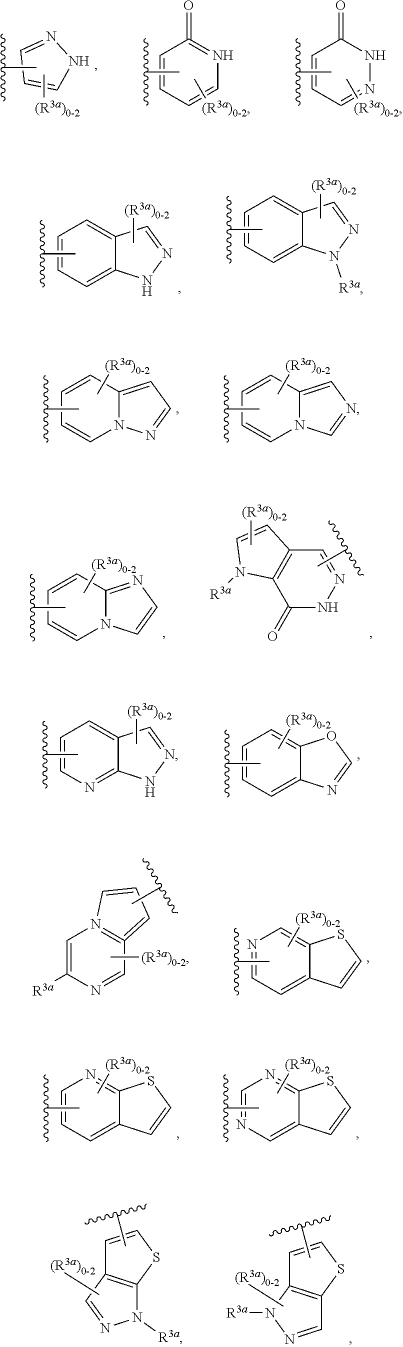

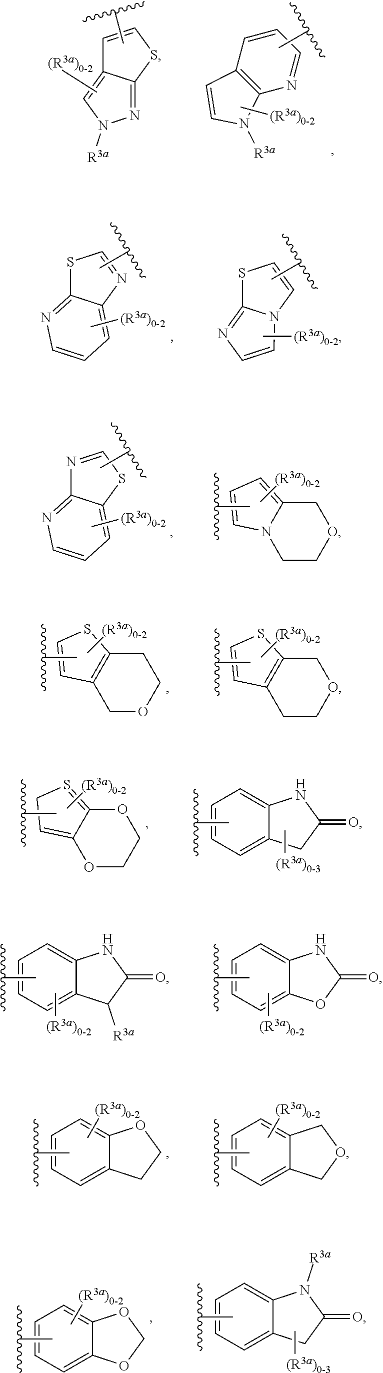

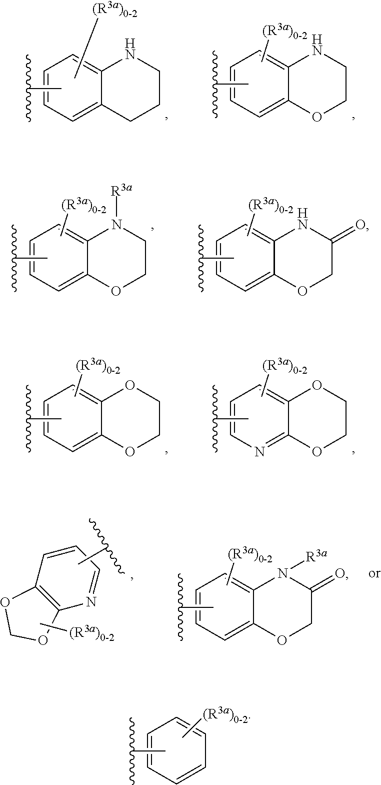

In some embodiments, R³ is:

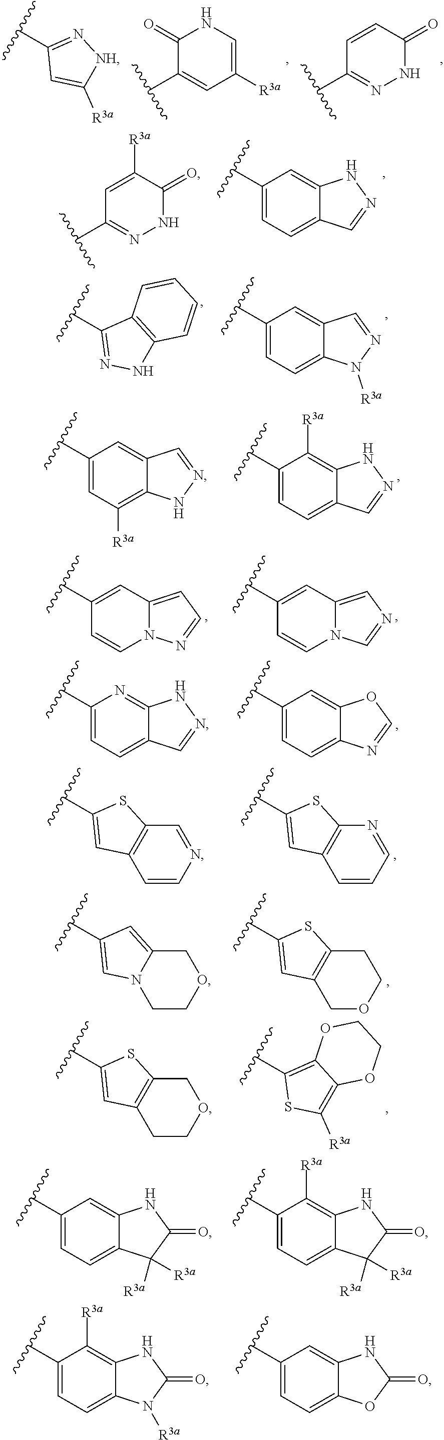

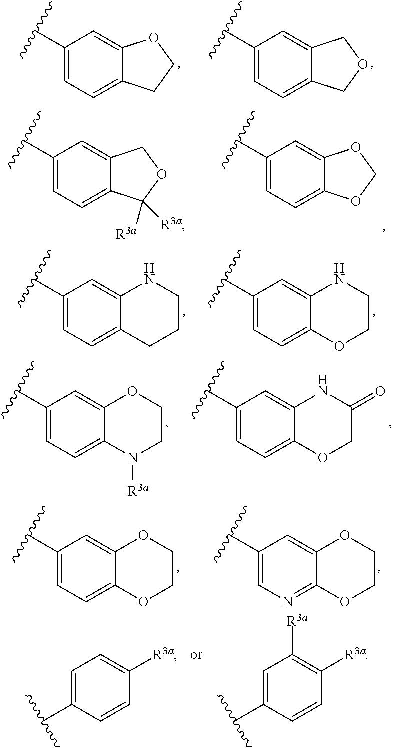

In some embodiments, R³ is:

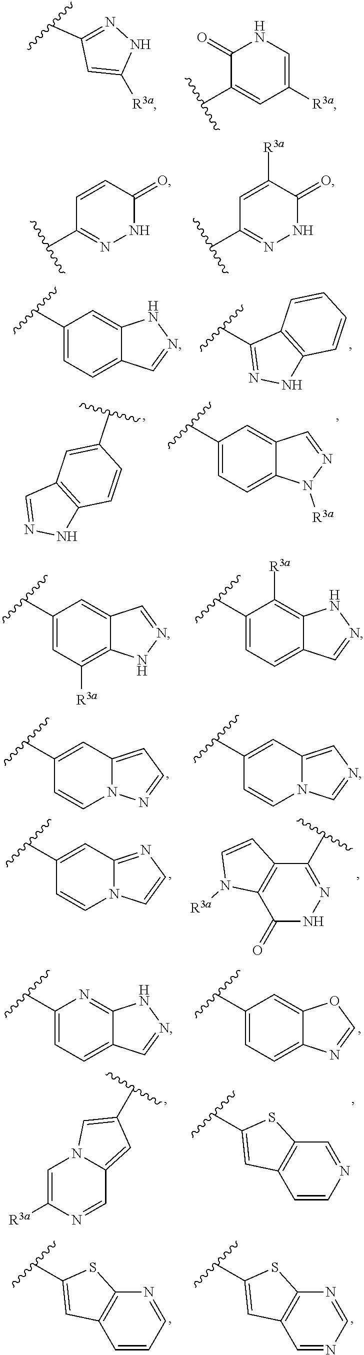

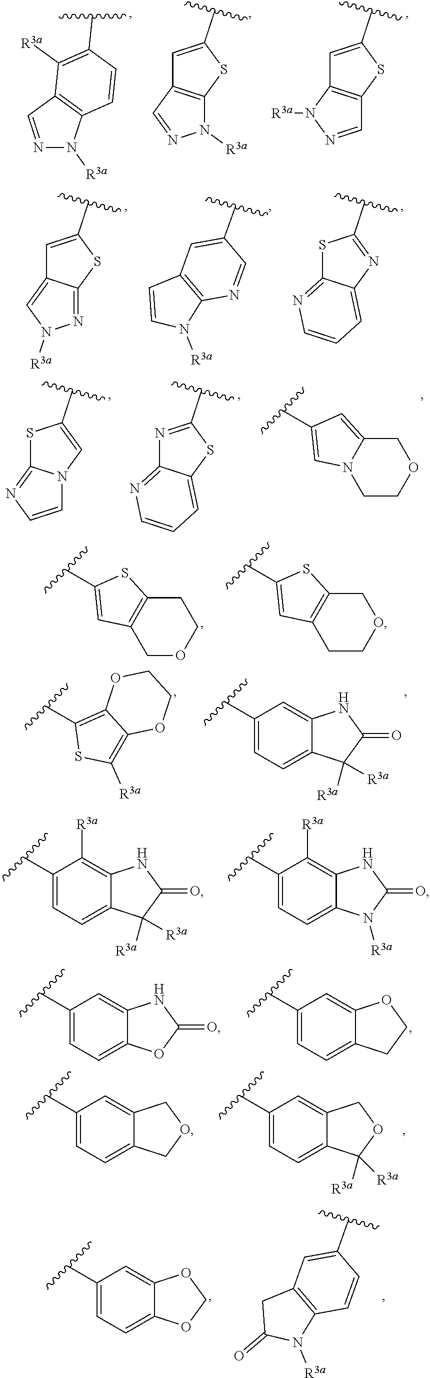

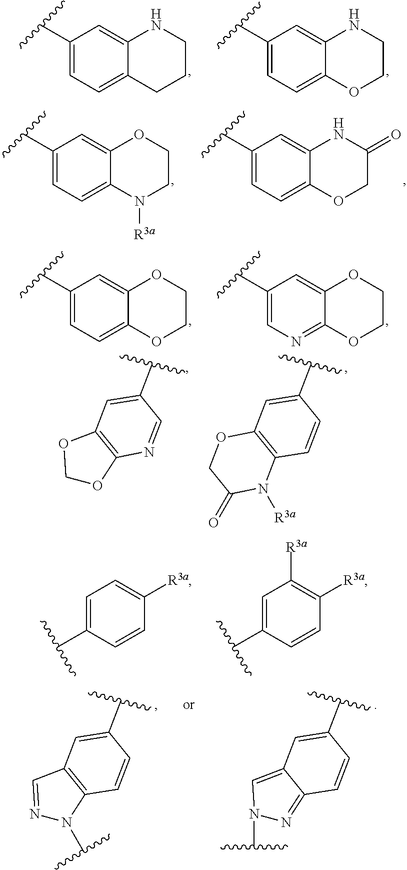

In some embodiments, R³ is:

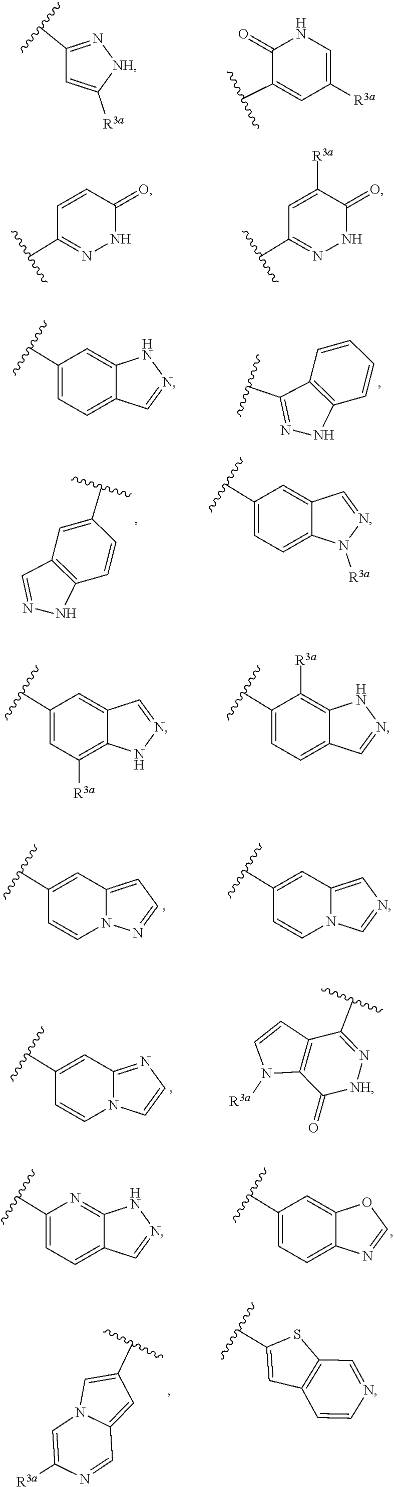

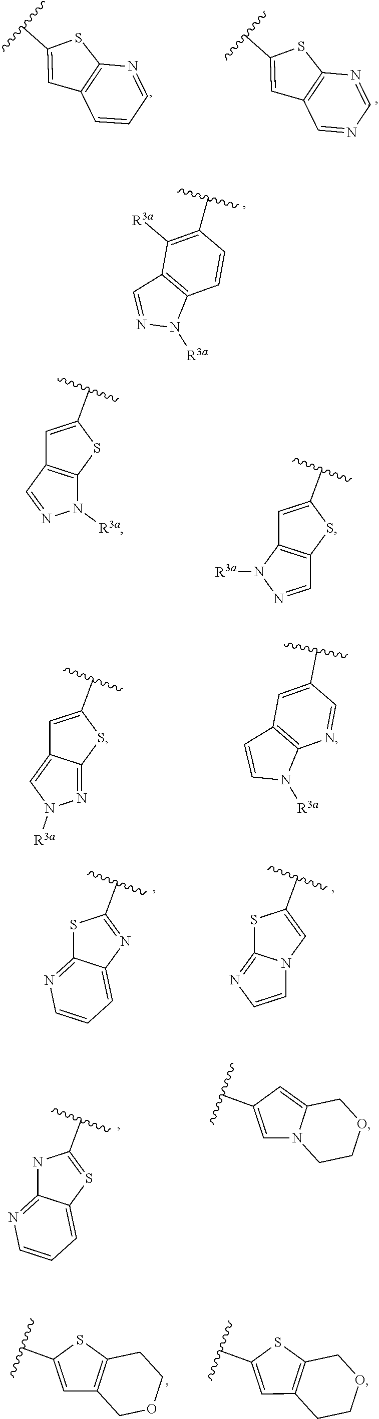

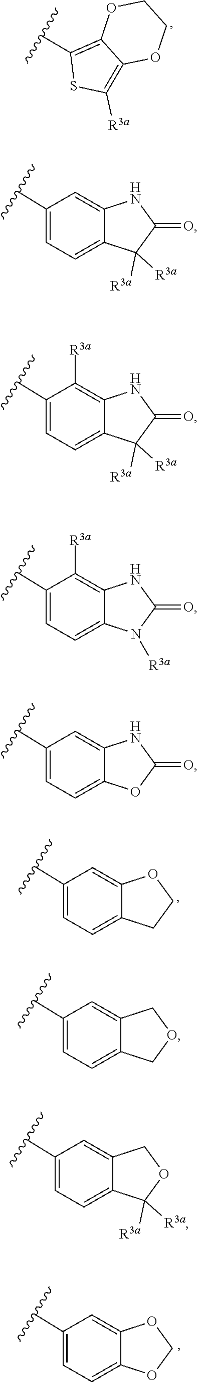

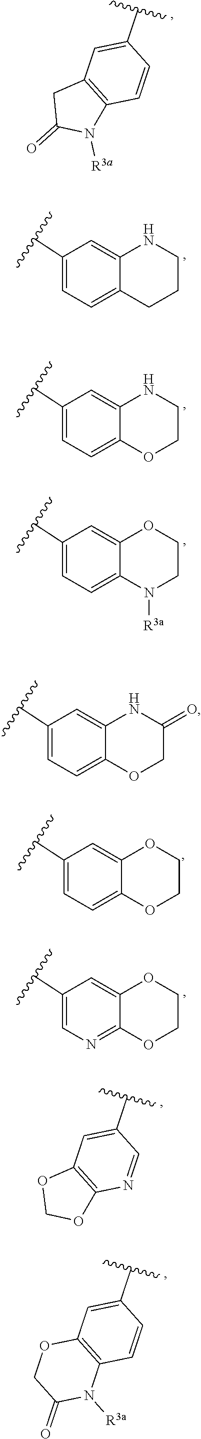

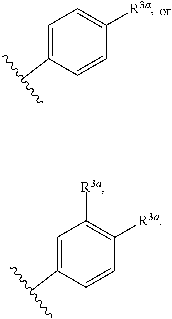

In some embodiments, R³ optionally substituted with one or more instances of R^{3a} is:

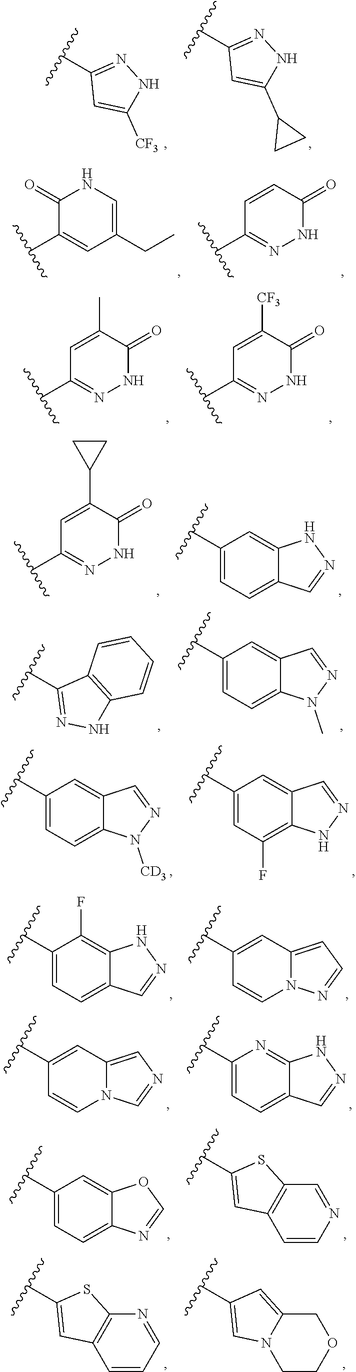

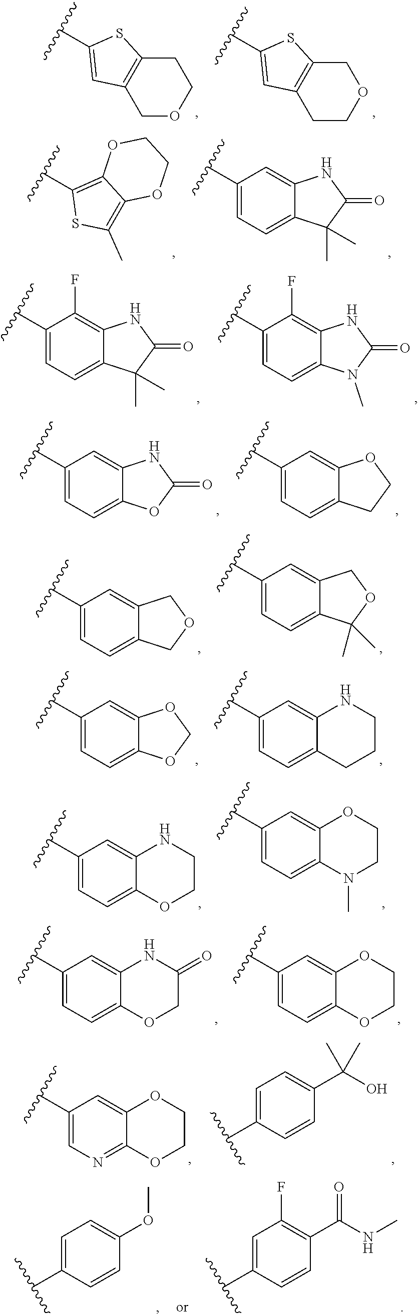

In some embodiments, R³ optionally substituted with one or more instances of R^{3a} is:

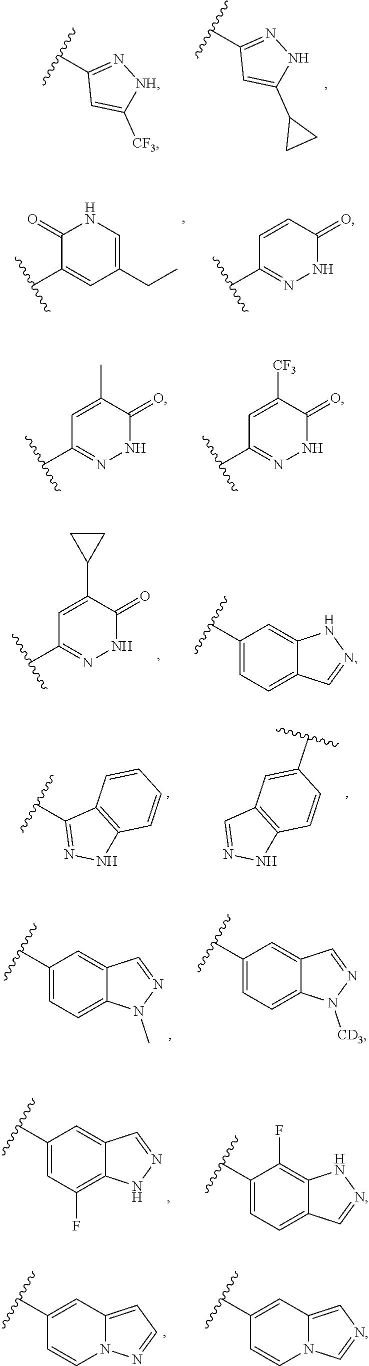

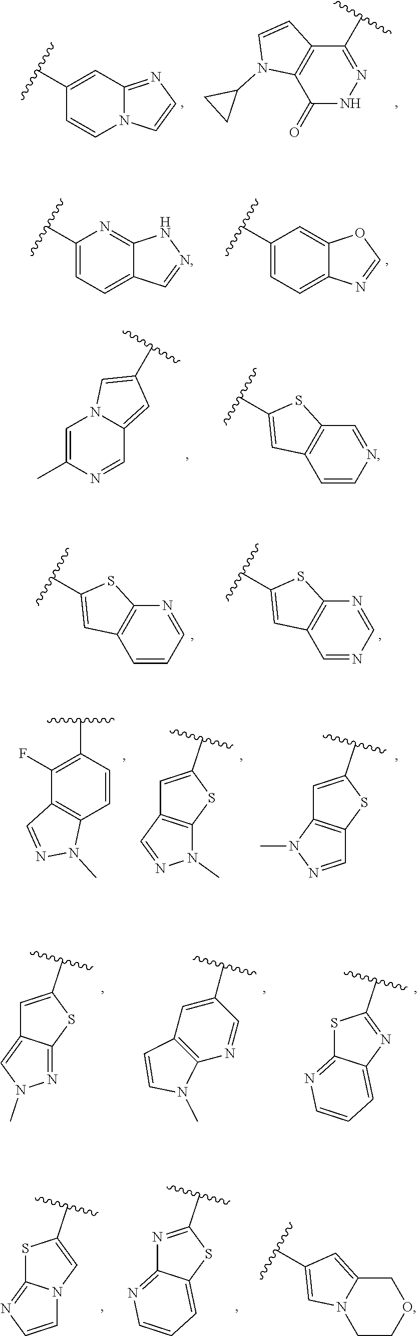

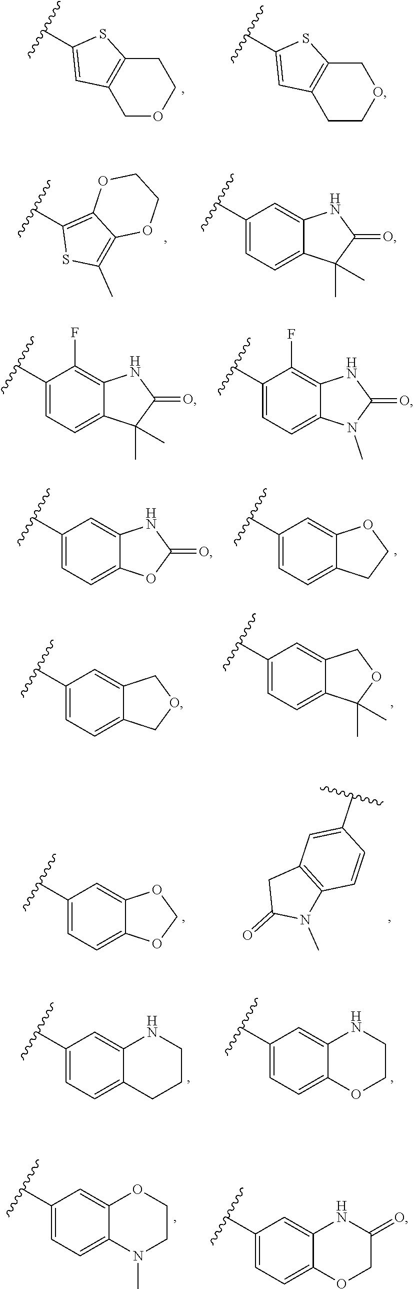

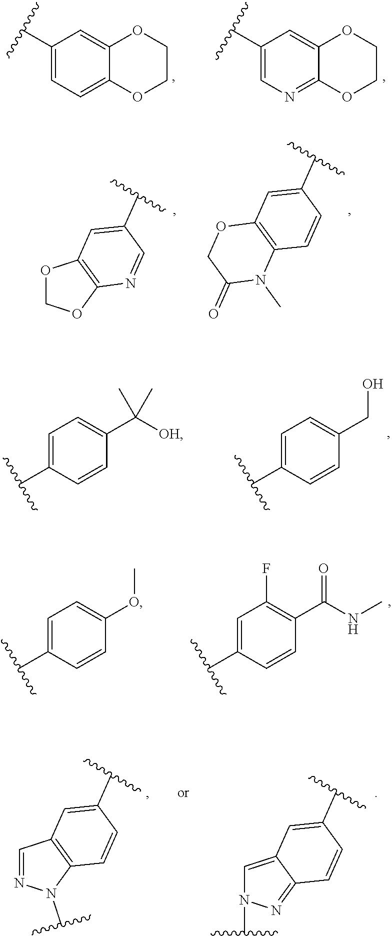

In some embodiments, R³ optionally substituted with one or more instances of R^{3a} is:

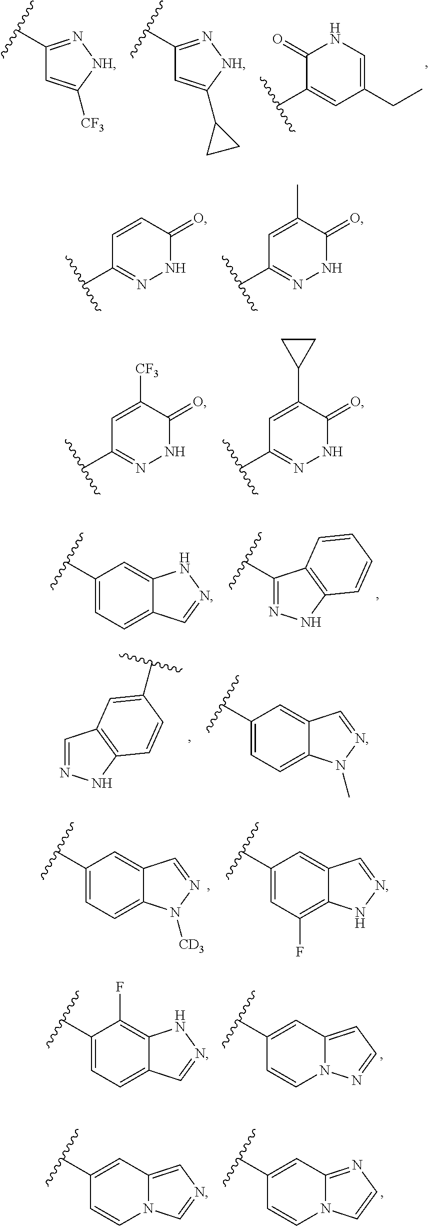

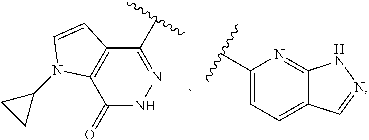 [SMILES: *c1n[nH]c(=O)c2c1ccn2C1CC1 ; *c1ccc2cn[nH]c2n1]

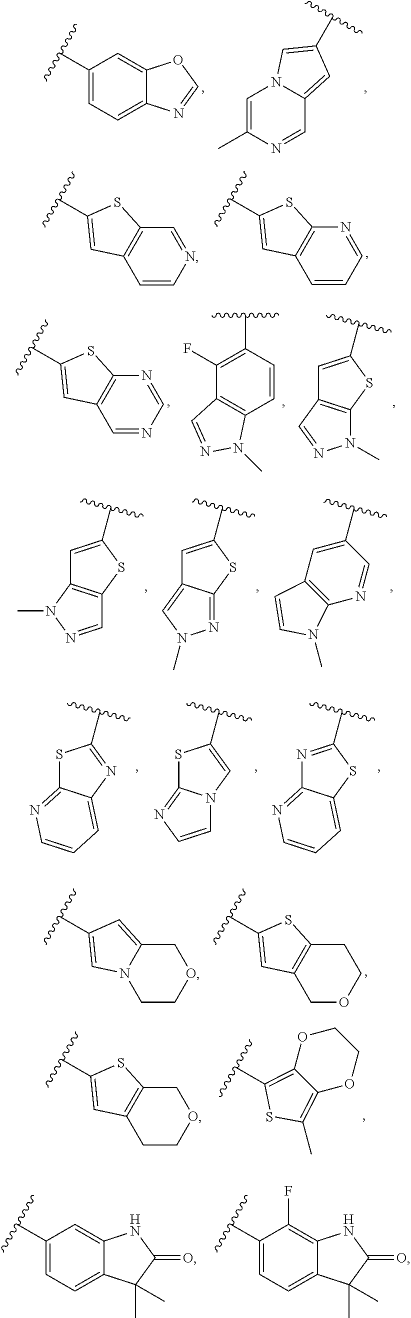

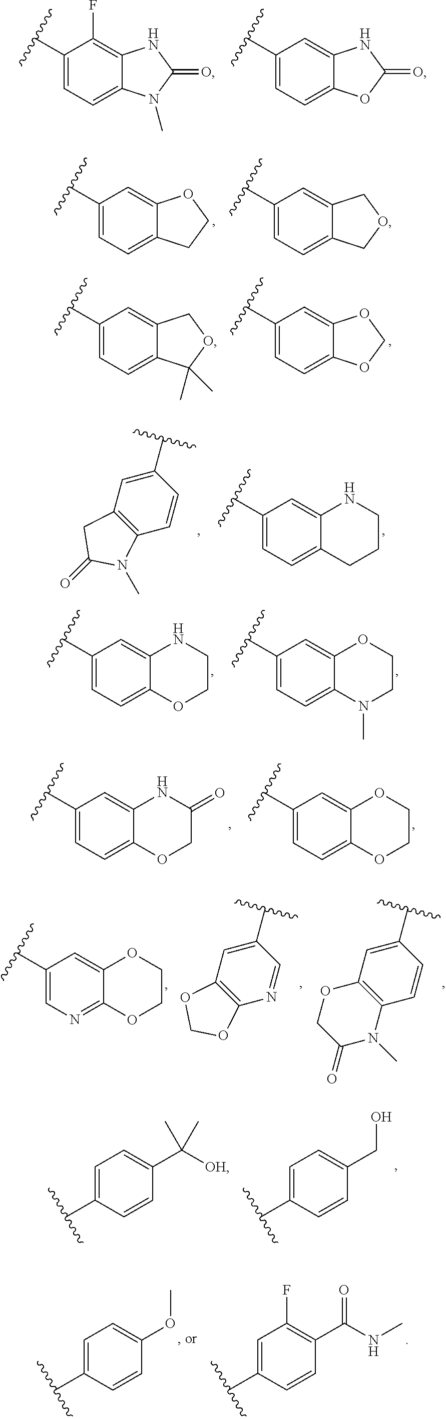

As described herein, with respect to any formula provided herein (e.g., any of Formulae I-IX), each R^{3a} is independently selected from the group consisting of optionally substituted C₁-C₆aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}. In some embodiments, each R^{3a} is independently selected from the group consisting of optionally substituted C₁-C₆ aliphatic, optionally substituted C₃-C₆ cycloaliphatic, optionally substituted 4- to 6-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —C(O)N(R^{a})₂, —N(R^{a})C(O)R^{a}, —S(O)₂N(R^{a})₂, —N(R^{a})S(O)₂R^{a}, S(O)₂R^{a}, and —OR^{a}. In some embodiments, each R^{3a} is independently selected from optionally substituted C₁-C₆ aliphatic, optionally substituted 2- to 10-membered heteroaliphatic, optionally substituted C₃-C₁₂ cycloaliphatic, and optionally substituted 4- to 10-membered heterocycle comprising 1 to 4 heteroatoms selected from N, O, and S. In some embodiments, each R^{3a} is independently selected from optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, halogen, and oxo. In some embodiments, each R^{3a} is independently selected from optionally substituted C₁-C₆ aliphatic, optionally substituted C₃-C₆ cycloaliphatic, halogen, —C(O)N(R^{a})₂, and —OR^{a}. In some embodiments, each R^{3a} is independently selected from optionally substituted C₁-C₆ aliphatic, optionally substituted C₃-C₆ cycloaliphatic, halogen, oxo, —C(O)N(R^{a})₂, and —OR^{a}. In some embodiments, each R^{3a} is independently selected from C₁-C₆ aliphatic optionally substituted with one or more —OR^{∘} or halogen, C₃-C₆ cycloaliphatic, halogen, —C(O)N(R^{a})₂, and —OR^{a}. In some embodiments, each R^{3a} is independently selected from C₁-C₆ aliphatic optionally substituted with one or more —OR^{∘} or halogen, C₃-C₆ cycloaliphatic, halogen, oxo, —C(O)N(R^{a})₂, and —OR^{a}.

In some embodiments, R^{3a} is optionally substituted C₁-C₆ aliphatic. In some embodiments, R^{3a} is optionally substituted C₁-C₆ alkyl. In some embodiments, R^{3a} is C₁-C₆ aliphatic optionally substituted with one or more —OR^{∘} or halogen. In some embodiments, R^{3a} is C₁-C₆ alkyl optionally substituted with one or more —OR^{∘} or halogen. In some embodiments, R^{3a} is methyl or deuterated methyl (e.g., one or more H atoms of methyl have been replaced with deuterium). In some embodiments, R^{3a} is a branched C₁-C₆ aliphatic. In some embodiments, R^{3a} is C₁-C₆ aliphatic optionally substituted with —(CH₂)₀₋₄OR^{∘}, wherein R^{∘} is hydrogen or C₁-C₆ aliphatic. In some embodiments, R^{3a} is —CH₃, —CD₃, —CF₃, —CH₂CH₃, or —C(CH₃)₂OH. In some embodiments, R^{3a} is —CH₃, —CD₃, —CF₃, —CH₂CH₃, —CH₂OH, or —C(CH₃)₂OH.

In some embodiments, R^{3a} is optionally substituted C₁-C₃ aliphatic.

In some embodiments, R^{3a} is optionally substituted C₃-C₁₂ cycloaliphatic. In some embodiments, R^{3a} is optionally substituted C₃-C₆ cycloaliphatic. In some embodiments, R^{3a} is C₃-C₆cycloaliphatic. In some embodiments, R^{3a} is optionally substituted C₃-C₆ cycloaliphatic wherein the C₃-C₆ cycloaliphatic is selected from cyclopropyl, cyclobutyl, cyclopentyl, and cyclohexyl.

In some embodiments, R^{3a} is optionally substituted 2- to 10-membered heteroaliphatic. In some embodiments, R^{3a} is methoxy. In some embodiments, R^{3a} is ethoxy.

In some embodiments, R^{3a} is optionally substituted 4- to 10-membered heterocycle comprising 1 to 4 heteroatoms selected from N, O, and S. In some embodiments, R^{3a} is 4- to 10-membered heterocycle comprising 1 to 4 heteroatoms selected from N, O, and S. In some embodiments, R^{3a} is optionally substituted 4- to 10-membered heterocycle comprising 1 to 2 heteroatoms selected from N, O, and S. In some embodiments, R^{3a} is optionally substituted 4- to 6-membered heterocycle comprising 1 to 4 heteroatoms selected from N, O, and S. In some embodiments, R^{3a} is a 4- to 6-membered heterocycle comprising 1 to 4 heteroatoms selected from N, O, and S. In some embodiments, R^{3a} is optionally substituted 4- to 6-membered heterocycle comprising 1 to 2 heteroatoms selected from N, O, and S.

In some embodiments, R^{3a} is optionally substituted 5- to 10-membered heteroaryl comprising 1 to 4 heteroatoms selected from N, O, and S. In some embodiments, R^{3a} is optionally substituted 5- to 6-membered heteroaryl comprising 1 to 3 heteroatoms selected from N, O, and S.

In some embodiments, R^{3a} is optionally substituted C₆-C₁₀ aryl. In some embodiments, R^{3a} is optionally substituted phenyl. In some embodiments, R^{3a} is optionally substituted naphthyl.

In some embodiments, R^{3a} is halogen. In some embodiments, R^{3a} is fluoro. In some embodiments, R^{3a} is chloro.

In some embodiments, R^{3a} is oxo. In some embodiments, R^{3a} is —CN. In some embodiments, R^{3a} is —NO₂. In some embodiments, R^{3a} is —C(O)N(R^{a})₂. In some embodiments, R^{3a} is —C(O)NH₂. In some embodiments, R^{3a} is —C(O)N(H)CH₃. In some embodiments, R^{3a} is —C(O)N(CH₃)₂. In some embodiments, R^{3a} is —OC(O)R^{a}. In some embodiments, R^{3a} is —OC(O)CH₃. In some embodiments, R^{3a} is —OC(O)N(R^{a})₂. In some embodiments, R^{3a} is —OC(O)NH₂. In some embodiments, R^{3a} is —OC(O)N(CH₃)₂. In some embodiments, R^{3a} is —N(R^{a})C(O)R^{a}. In some embodiments, R^{3a} is —NHC(O)CH₃. In some embodiments, R^{3a} is —N(CH₃)C(O)CH₃. In some embodiments, R^{3a} is —N(R^{a})C(O)OR^{a}. In some embodiments, R^{3a} is —NHC(O)OH. In some embodiments, R^{3a} is —NHC(O)OCH₃. In some embodiments, R^{3a} is —N(R^{a})C(O)N(R^{a})₂. In some embodiments, R^{3a} is —NHC(O)NH₂. In some embodiments, R^{3a} is —NHC(O)N(CH₃)₂. In some embodiments, R^{3a} is —S(O)₂N(R^{a})₂. In some embodiments, R^{3a} is —S(O)₂NH₂. In some embodiments, R^{3a} is —S(O)₂N(CH₃)₂. In some embodiments, R^{3a} is —N(R^{a})S(O)₂R^{a}. In some embodiments, R^{3a} is —NHS(O)₂CH₃. In some embodiments, R^{3a} is —S(O)₂R^{a}. In some embodiments, R^{3a} is —OR^{a}. In some embodiments, R^{3a} is —O(C₁₋₃ alkyl), e.g., —OCH₃. In some embodiments, R^{3a} is —OH. In some embodiments, R^{3a} is —CH₃. In some embodiments, R^{3a} is —CD₃.

In some embodiments, R^{3a} is —CH₃, —CD₃,

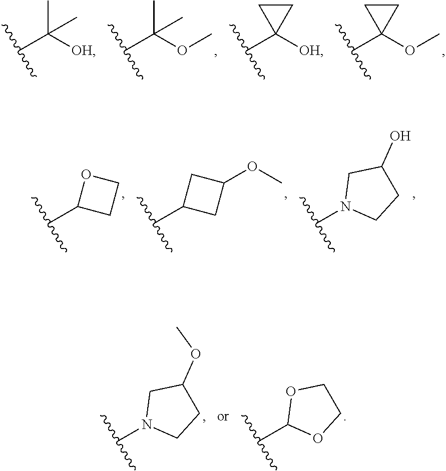

In some embodiments, R^{3a} is —F, oxo, cyclopropyl, —CH₃, —CD₃,

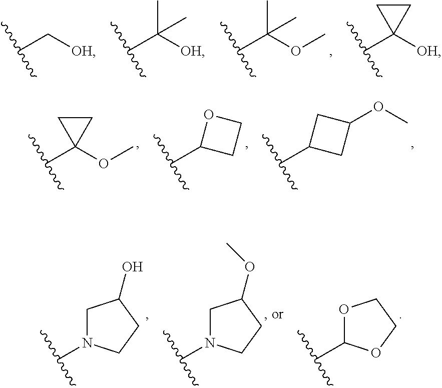

In some embodiments, each R^{3a} is independently —CH₃, —CD₃, —CF₃, —CH₂CH₃,

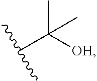 [SMILES: *C(C)(C)O]

—OCH₃, fluoro,

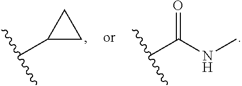

In some embodiments, each R^{3a} is independently oxo, —CH₃, —CD₃, —CF₃, —CH₂CH₃,

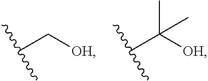

—OCH₃, fluoro,

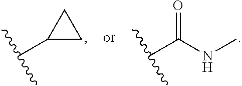

In some embodiments, R³ is

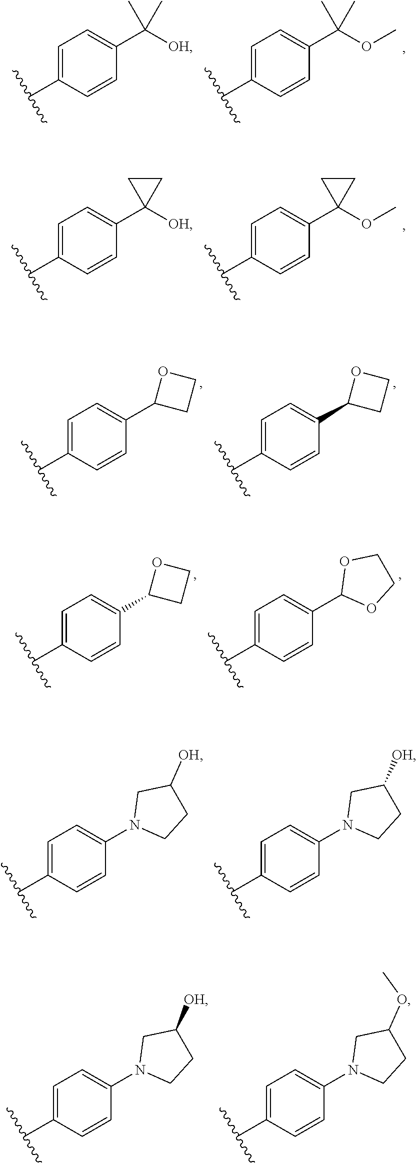

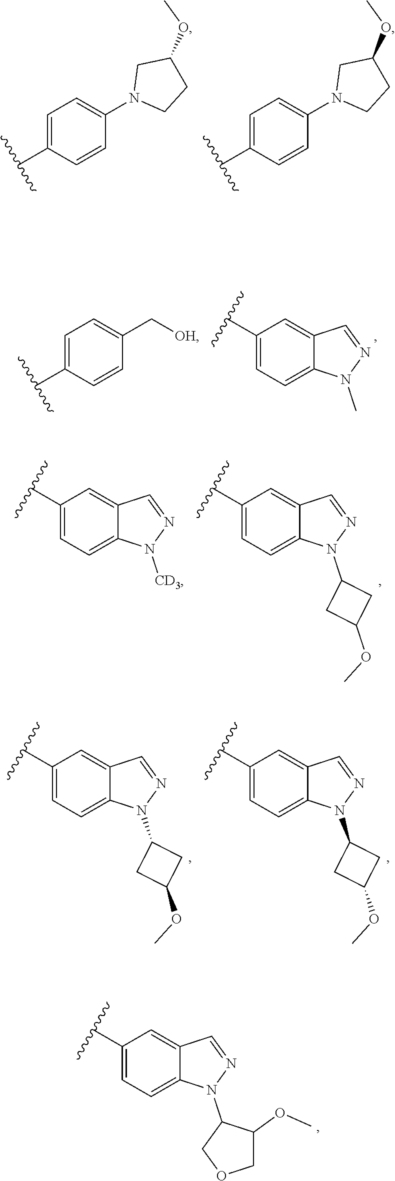

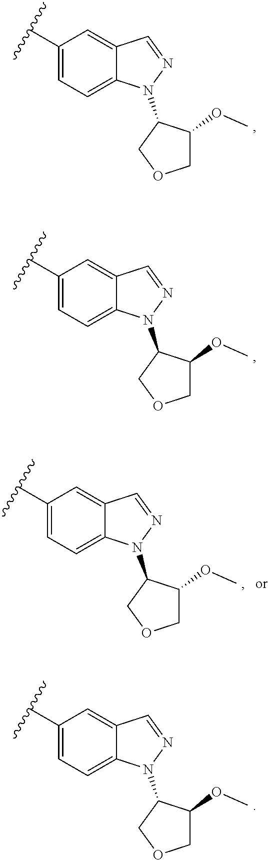 [SMILES: *c1ccc2c(cnn2[C@H]2COC[C@H]2OC)c1 ; *c1ccc2c(cnn2[C@@H]2COC[C@@H]2OC)c1 ; *c1ccc2c(cnn2[C@@H]2COC[C@H]2OC)c1 ; *c1ccc2c(cnn2[C@H]2COC[C@@H]2OC)c1]

In some embodiments, R² and R³ are connected by *-L^{a}-R⁶-L^{b}-**, where * represents a point of attachment to R², and ** represents a point of attachment to R³.

As described herein, with respect to any formula provided herein (e.g., any of Formulae I-IX), L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—. In some embodiments, L^{a} is a bond, —N(R^{a})C(O)O—, —OC(O)N(R^{a})—, —C(O)N(R^{a})—, —N(R^{a})C(O)—, —N(R^{a})—, —O—, —C(O)—, or —C(O)O—. In some embodiments, L^{a} is a bond, —N(H)C(O)O—, —OC(O)N(H)—, —C(O)N(H)—, or —N(H)C(O)—.

In some embodiments, L^{a} is a bond. In some embodiments, L^{a} is —N(R^{a})C(O)N(R^{a})—. In some embodiments, L^{a} is —NHC(O)NH—. In some embodiments, L^{a} is —N(R^{a})C(O)O—. In some embodiments, L^{a} is —NHC(O)O—. In some embodiments, L^{a} is —OC(O)N(R^{a})—. In some embodiments, L^{a} is —OC(O)NH—. In some embodiments, L^{a} is —C(O)N(R^{a})—. In some embodiments, L^{a} is —C(O)NH—. In some embodiments, L^{a} is —N(R^{a})C(O)—. In some embodiments, L^{a} is —NHC(O)—. In some embodiments, L^{a} is —N(R^{a})—. In some embodiments, L^{a} is —NH—. In some embodiments, L^{a} is —O—. In some embodiments, L^{a} is —C(O)—. In some embodiments, L^{a} is —C(O)O—. In some embodiments, L^{a} is —OC(O)—. In some embodiments, L^{a} is —N(R^{a})C(S)O—. In some embodiments, L^{a} is —NHC(S)O—. In some embodiments, L^{a} is —OC(S)N(R^{a})—. In some embodiments, L^{a} is —OC(S)NH—. In some embodiments, L^{a} is —C(S)N(R^{a})—. In some embodiments, L^{a} is —C(S)NH—. In some embodiments, L^{a} is —N(R^{a})C(S)—. In some embodiments, L^{a} is —NHC(S)—. In some embodiments, L^{a} is —C(S)—. In some embodiments, L^{a} is —C(S)O—. In some embodiments, L^{a} is —OC(S)—.

As described herein, with respect to any formula provided herein (e.g., any of Formulae I-IX), L^{b} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—. In some embodiments, L^{b} is a bond, —N(R^{a})—, —O—, —C(O)—, —C(O)O—, or —OC(O)—.

In some embodiments, L^{b} is a bond. In some embodiments, L^{b} is —N(R^{a})C(O)N(R^{a})—. In some embodiments, L^{b} is —NHC(O)NH—. In some embodiments, L^{b} is —N(R^{a})C(O)O—. In some embodiments, L^{b} is —NHC(O)O—. In some embodiments, L^{b} is —OC(O)N(R^{a})—. In some embodiments, L^{b} is —OC(O)NH—. In some embodiments, L^{b} is —C(O)N(R^{a})—. In some embodiments, L^{b} is —C(O)NH—. In some embodiments, L^{b} is —N(R^{a})C(O)—. In some embodiments, L^{b} is —NHC(O)—. In some embodiments, L^{b} is —N(R^{a})—. In some embodiments, L^{b} is —NH—. In some embodiments, L^{b} is —O—. In some embodiments, L^{b} is —C(O)—. In some embodiments, L^{b} is —C(O)O—. In some embodiments, L^{b} is —OC(O)—. In some embodiments, L^{b} is —N(R^{a})C(S)O—. In some embodiments, L^{b} is —NHC(S)O—. In some embodiments, L^{b} is —OC(S)N(R^{a})—. In some embodiments, L^{b} is —OC(S)NH—. In some embodiments, L^{b} is —C(S)N(R^{a})—. In some embodiments, L^{b} is —C(S)NH—. In some embodiments, L^{b} is —N(R^{a})C(S)—. In some embodiments, L^{b} is —NHC(S)—. In some embodiments, L^{b} is —C(S)—. In some embodiments, L^{b} is —C(S)O—. In some embodiments, L^{b} is —OC(S)—.

In some embodiments, L^{a} is —N(R^{a})C(O)O— or —C(O)N(R^{a})—, and L^{b} is a bond. In some embodiments, L^{a} is —N(H)C(O)O— and L^{b} is a bond. In some embodiments, L^{a} is —N(R^{a})C(O)O—, —C(O)N(R^{a})—, or —O— and L^{b} is a bond. In some embodiments, L^{a} is —N(H)C(O)O— or —O— and L^{b} is a bond. In some embodiments, L^{a} is —O— and L^{b} is a bond

As described herein, with respect to any formula provided herein (e.g., any of Formulae I-IX), R⁶ is optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, or optionally substituted C₆-C₁₀ aryl. In some embodiments, R⁶ is optionally substituted C₁-C₆ aliphatic, optionally substituted 2- to 10-membered heteroaliphatic, optionally substituted C₃-C₁₂ cycloaliphatic, or optionally substituted 4- to 10-membered heterocycle comprising 1 to 4 heteroatoms selected from N, O, and S. In some embodiments, R⁶ is optionally substituted C₁-C₆ aliphatic or optionally substituted 2- to 10-membered heteroaliphatic.

In some embodiments, R⁶ is optionally substituted C₁-C₆ aliphatic. In some embodiments, R⁶ is optionally substituted C₁-C₃ aliphatic. In some embodiments, R⁶ is a branched C₁-C₆ aliphatic. In some embodiments, R⁶ is methyl, ethyl, propyl, butyl, pentyl, or hexyl.

In some embodiments, R⁶ is optionally substituted C₃-C₁₂ cycloaliphatic. In some embodiments, R⁶ is optionally substituted C₃-C₆ cycloaliphatic. In some embodiments, R⁶ is optionally substituted C₃-C₆ cycloaliphatic, wherein the C₃-C₆ cycloaliphatic is selected from cyclopropyl, cyclobutyl, cyclopentyl, and cyclohexyl.

In some embodiments, R⁶ is optionally substituted 2- to 10-membered heteroaliphatic. In some embodiments, R⁶ is optionally substituted C₁-C₃ heteroaliphatic. In some embodiments, R⁶ is methoxy. In some embodiments, R⁶ is ethoxy.

In some embodiments, R⁶ is optionally substituted 4- to 10-membered heterocycle comprising 1 to 4 heteroatoms selected from N, O, and S. In some embodiments, R⁶ is 4- to 10-membered heterocycle comprising 1 to 4 heteroatoms selected from N, O, and S. In some embodiments, R⁶ is optionally substituted 4- to 6-membered heterocycle comprising 1 to 4 heteroatoms selected from N, O, and S. In some embodiments, R⁶ is a 4- to 6-membered heterocycle comprising 1 to 4 heteroatoms selected from N, O, and S. In some embodiments, R⁶ is optionally substituted 4- to 6-membered heterocycle comprising 1 to 2 heteroatoms selected from N, O, and S. In some embodiments, R⁶ is a 4- to 6-membered heterocycle comprising 1 to 2 heteroatoms selected from N, O, and S.

In some embodiments, R⁶ is optionally substituted 5- to 10-membered heteroaryl comprising 1 to 4 heteroatoms selected from N, O, and S. In some embodiments, R⁶ is optionally substituted 5- to 6-membered heteroaryl comprising 1 to 3 heteroatoms selected from N, O, and S.

In some embodiments, R⁶ is optionally substituted C₆-C₁₀ aryl. In some embodiments, R⁶ is optionally substituted phenyl. In some embodiments, R⁶ is optionally substituted naphthyl.

In some embodiments, a moiety

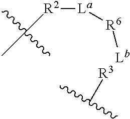

is

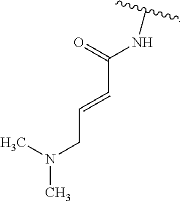 [SMILES: *NC(=O)/C=C/CN(C)C]

In some embodiments, a moiety

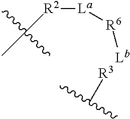

is

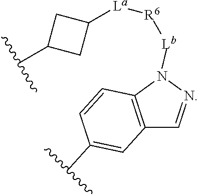

In some embodiments, a moiety

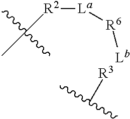

is

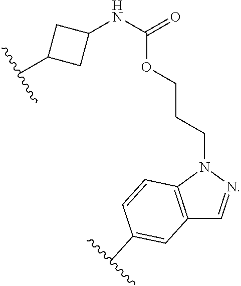 [SMILES: *c1ccc2c(cnn2CCCOC(=O)NC2CC(*)C2)c1]

In some embodiments, a moiety

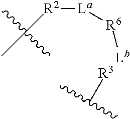

is

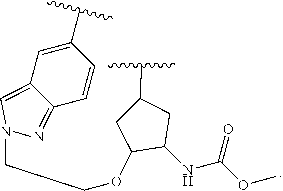 [SMILES: *c1ccc2nn(CCOC3CC(*)CC3NC(=O)OC)cc2c1]

In some embodiments, a moiety

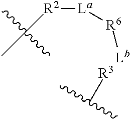

is

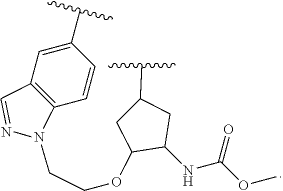 [SMILES: *c1ccc2c(cnn2CCOC2CC(*)CC2NC(=O)OC)c1]

It will be appreciated that R⁶ is bivalent (i.e., R⁶ has two points of attachment to the rest of the molecule) and, therefore, any definitions of R⁶ throughout this disclosure refer to a bivalent chemical moiety.

As described with respect to any formula provided herein (e.g., any of Formulae I-IX), R⁴ is selected from C₁-C₆ aliphatic, —CN, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, C₆-C₁₀ aryl, and wherein R⁴ is optionally substituted with one or more instances of R^{4a}. In some embodiments, R⁴ is selected from C₁-C₆ aliphatic, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, C₆-C₁₀ aryl, and wherein R⁴ is optionally substituted with one or more instances of R^{4a}. In some embodiments, R⁴ is selected from C₁-C₆ aliphatic, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, C₆-C₁₀ aryl, and wherein R⁴ is optionally substituted with 0-3 instances of R^{4a}. In some embodiments, R⁴ is selected from C₁-C₆ aliphatic, —CN, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, C₆-C₁₀ aryl, and wherein R⁴ is optionally substituted with 0-3 instances of R^{4a}. In some embodiments, R⁴ is 5- to 10-membered heteroaryl comprising 1 to 4 heteroatoms selected from N, O, and S or 4- to 10-membered heterocycle comprising 1 to 4 heteroatoms selected from N, O, and S, and wherein R⁴ is optionally substituted with one or more instances of R^{4a}. In some embodiments, R⁴ is 5- to 10-membered heteroaryl comprising 1 to 4 heteroatoms selected from N, O, and S or 4- to 10-membered heterocycle comprising 1 to 4 heteroatoms selected from N, O, and S, and wherein R⁴ is optionally substituted with 0-3 instances of R^{4a}.

In some embodiments, R⁴ is C₁-C₆ aliphatic optionally substituted with one or more instances of R^{4a}. In some embodiments, R⁴ is C₁-C₃ aliphatic optionally substituted with one or more instances of R^{4a}. In some embodiments, R⁴ is C₁-C₆ aliphatic optionally substituted with one or two instances of R^{4a}.

In some embodiments, R⁴ is —CN.

In some embodiments, R⁴ is 2- to 10-membered heteroaliphatic optionally substituted with one or more instances of R^{4a}.

In some embodiments, R⁴ is C₃-C₁₂ cycloaliphatic optionally substituted with one or more instances of R^{4a}. In some embodiments, R⁴ is C₃-C₆ cycloaliphatic. In some embodiments, R⁴ is monocyclic 3- to 7-membered cycloaliphatic optionally substituted with one or more instances of R^{4a}. In some embodiments, R⁴ is cyclopropyl, cyclobutyl, cyclopentyl, cyclohexyl, or cycloheptyl. In some embodiments, R⁴ is cyclopropyl.

In some embodiments, R⁴ is 4- to 10-membered heterocycle comprising 1 to 4 heteroatoms selected from N, O, and S optionally substituted with one or more instances of R^{4a}. In some embodiments, R⁴ is 4- to 6-membered monocyclic heterocycle comprising 1 to 3 heteroatoms selected from N, O, and S. In some embodiments, R⁴ is 4- to 6-membered monocyclic heterocycle comprising 1 to 3 heteroatoms selected from N, O, and S optionally substituted with one or more instances of R^{4a}. In some embodiments, R⁴ is 4- to 6-membered monocyclic heterocycle comprising 1 to 3 heteroatoms selected from N, O, and S substituted with one or more instances of R^{4a}, wherein each R^{4a} is independently halogen, —CH₃, or -CD₃. In some embodiments, R⁴ is piperidine or pyran optionally substituted with one or more instances of R^{4a}. In some embodiments, R⁴ is piperidine or pyran. In some embodiments, R⁴ is a tetrahydropyran or dihydropyran optionally substituted with 0-3 instances of R^{4a}. In some embodiments, R⁴ is a tetrahydropyran or dihydropyran.

In some embodiments, R⁴ is 6- to 10-membered bicyclic heterocycle comprising 1 to 4 heteroatoms selected from N, O, and S. In some embodiments, R⁴ is 6- to 10-membered bicyclic heterocycle comprising 1 to 4 heteroatoms selected from N, O, and S optionally substituted with one or more instances of R^{4a}. In some embodiments, R⁴ is 8- to 10-membered bicyclic heterocycle comprising 1 to 4 heteroatoms selected from N and O, optionally substituted with one or more instances of R^{4a}.

In some embodiments, R⁴ is 6- to 10-membered spirocyclic heterocycle comprising 1 to 4 heteroatoms selected from N, O, and S. In some embodiments, R⁴ is 6- to 10-membered spirocyclic heterocycle comprising 1 to 4 heteroatoms selected from N, O, and S optionally substituted with one or more instances of R^{4a}. In some embodiments, R⁴ is 6- to 10-membered spirocyclic heterocycle comprising 1 to 2 heteroatoms selected from N and O, optionally substituted with one or more instances of R^{4a}.

In some embodiments, R⁴ is 5- to 10-membered heteroaryl comprising 1 to 4 heteroatoms selected from N, O, and S, and is optionally substituted with one or more instances of R^{4a}. In some embodiments, R⁴ is monocyclic 5- to 6-membered heteroaryl comprising 1 to 3 heteroatoms selected from N, O, and S, and is optionally substituted with one or more instances of R^{4a}. In some embodiments, R⁴ is monocyclic 5-membered heteroaryl comprising 1 to 3 heteroatoms selected from N, O, and S, and is optionally substituted with one or more instances of R^{4a}. In some embodiments, R⁴ is monocyclic 6-membered heteroaryl comprising 1 to 3 heteroatoms selected from N, O, and S, and is optionally substituted with one or more instances of R^{4a}. In some embodiments, R⁴ is 5- to 6-membered monocyclic heteroaryl comprising 1 to 3 heteroatoms selected from N, O, and S and is substituted with one instance of R^{4a}. In some embodiments, R⁴ is 5- to 6-membered monocyclic heteroaryl comprising 1 to 3 heteroatoms selected from N, O, and S, and wherein R⁴ is optionally substituted with one or more instances of R^{4a}, wherein R^{4a} is selected from optionally substituted C₁-C₆ aliphatic, —OR^{a}, and halogen. In some embodiments, R⁴ is 5- to 6-membered monocyclic heteroaryl comprising 1 to 3 heteroatoms selected from N, O, and S, and wherein R⁴ is optionally substituted with one or more instances of R^{4a}, wherein each R^{4a} is independently selected from optionally substituted C₁-C₆ aliphatic, optionally substituted 2- to 10-membered heteroaliphatic, —OR^{a}, —C(O)R^{a}, and halogen. In some embodiments, R⁴ is 5- to 6-membered monocyclic heteroaryl comprising 1 to 3 heteroatoms selected from N, O, and S, and wherein R⁴ is optionally substituted with R^{4a}, wherein R^{4a} is selected from optionally substituted C₁-C₆ aliphatic and halogen. In some embodiments, R⁴ is 5- to 6-membered monocyclic heteroaryl comprising 1 to 3 heteroatoms selected from N, O, and S, and wherein R⁴ is substituted with one or more instances of R^{4a}, wherein R^{4a} is selected from optionally substituted C₁-C₆ aliphatic and halogen. In some embodiments, R⁴ is 5- to 6-membered monocyclic heteroaryl comprising 1 to 3 heteroatoms selected from N, O, and S, and wherein R⁴ is optionally substituted with R^{4a}, wherein R^{4a} is selected from —CH₃ or -CD₃. In some embodiments, R⁴ is a pyrazole, triazole, thiazole, thiadiazole, oxazole, pyridazine, pyrimidine, or pyrazine, and wherein R⁴ is optionally substituted with one or more instances of R^{4a}. In some embodiments, R⁴ is pyrrole, pyrazole, imidazole, triazole, or thiazole, optionally substituted with R^{4a}. In some embodiments, R⁴ is pyrazole optionally substituted with R^{4a}. In some embodiments, R⁴ is pyrazole optionally substituted with R^{4a}, and wherein R^{4a} is —CH₃, or -CD₃.

In some embodiments, R⁴ is:

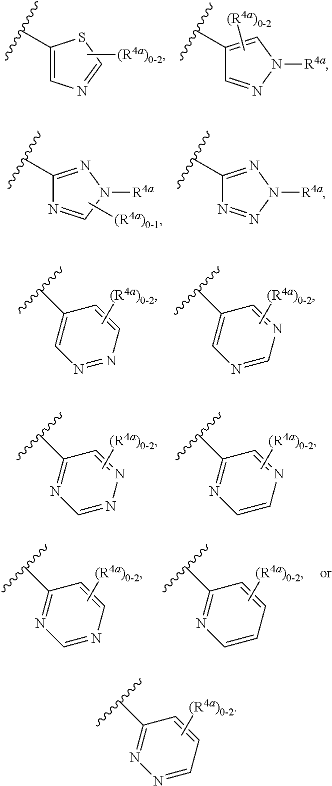

In some embodiments, R⁴ is:

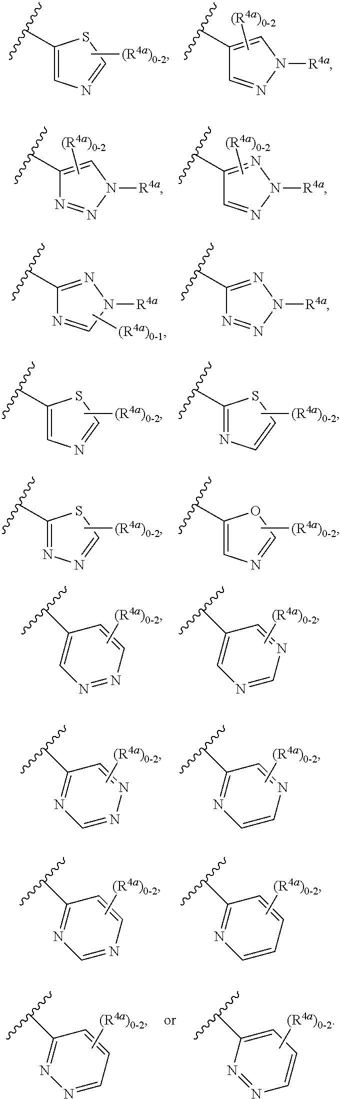

In some embodiments, R⁴ is:

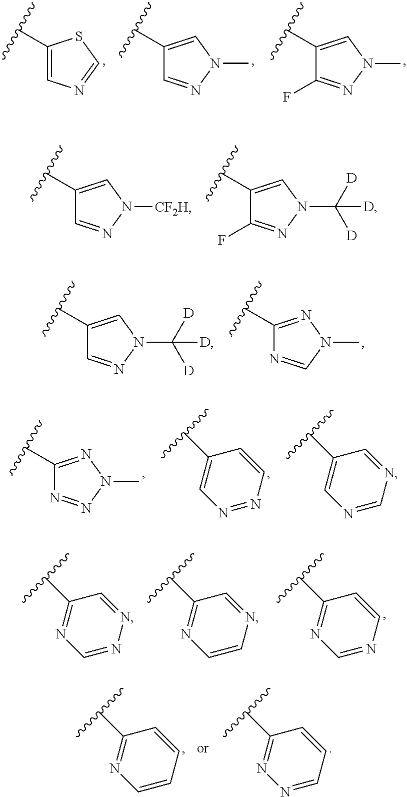

In some embodiments, R⁴ is:

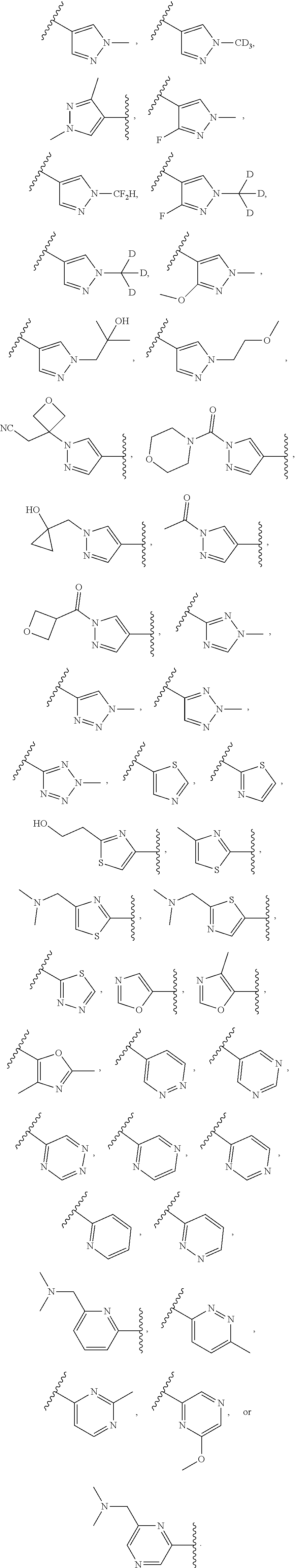

In some embodiments, R⁴ is bicyclic 7- to 10-membered heteroaryl comprising 1 to 4 heteroatoms selected from N, O, and S and is optionally substituted with one or more instances of R^{4a}. In some embodiments, R⁴ is bicyclic 7- to 10-membered heteroaryl comprising 1 to 3 heteroatoms selected from N, O, and S. In some embodiments, R⁴ is bicyclic 7- to 10-membered heteroaryl comprising 1 to 4 heteroatoms selected from N, O, and S, and is optionally substituted with R^{4a}. In some embodiments, R⁴ is bicyclic 7- to 10-membered heteroaryl comprising 1 to 3 heteroatoms selected from N, O, and S optionally substituted with one or more instances of R^{4a}. In some embodiments, R⁴ is 8- to 10-membered bicyclic heteroaryl having 1 to 4 heteroatoms selected from N, O, and S and is optionally substituted with one or more instances of R^{4a}. In some embodiments, R⁴ is 8- to 10-membered bicyclic heteroaryl having 1 to 4 heteroatoms selected from N and O and is optionally substituted with one or more instances of R^{4a}. In some embodiments, R⁴ is 8- to 10-membered bicyclic heteroaryl having 1 to 3 heteroatoms selected from N and O. In some embodiments, R⁴ is 8- to 10-membered bicyclic heteroaryl having 1 to 3 heteroatoms selected from N and O. In some embodiments, R⁴ is

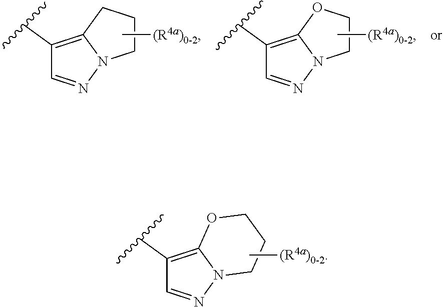

some embodiments, R⁴ is

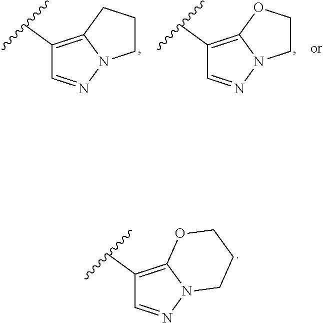

In some embodiments, R⁴ is C₆-C₁₀ aryl, and wherein R⁴ is optionally substituted with one or more instances of R^{4a}. In some embodiments, R⁴ is C₆-C₁₀ aryl. In some embodiments, R⁴ is phenyl optionally substituted with one or more instances of R^{4a}. In some embodiments, R⁴ is phenyl. In some embodiments, R⁴ is naphthyl.

In some embodiments, R⁴ is substituted with 0 to 4 instances of R^{4a}. In some embodiments, R⁴ is substituted with 0 to 3 instances of R^{4a}. In some embodiments, R⁴ is substituted with 0 to 2 instances of R^{4a}. In some embodiments, R⁴ is substituted with 1 to 3 instances of R^{4a}. In some embodiments, R⁴ is substituted with 1 to 2 instances of R^{4a}. In some embodiments, R⁴ is substituted with 1 instance of R^{4a}. In some embodiments, R⁴ is substituted with 2 instances of R^{4a}. In some embodiments, R⁴ is substituted with 3 instances of R^{4a}. In some embodiments, R⁴ is substituted with 4 instances of R^{4a}.

In some embodiments, R⁴ is selected from:

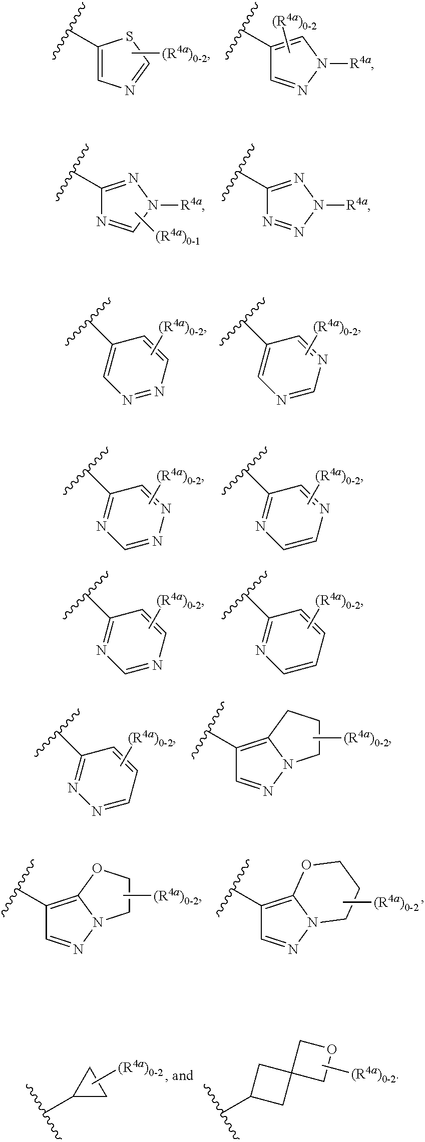

In some embodiments, R⁴ is selected from:

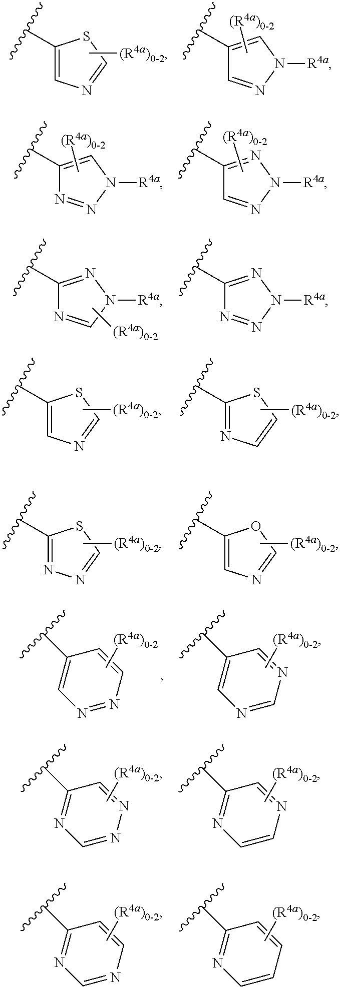

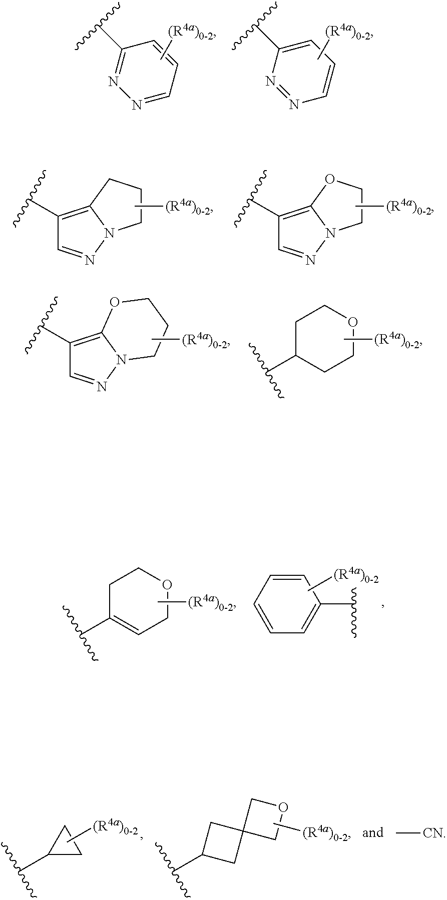

In some embodiments, R⁴ is

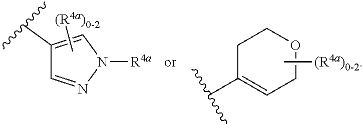

In some embodiments, R⁴ is

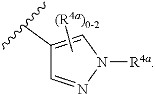

In some embodiments, R⁴ is selected from:

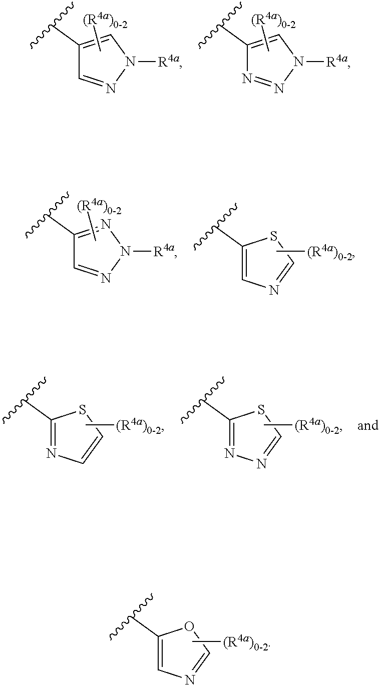

In some embodiments, R⁴ optionally substituted with one or more instances of R^{4a} is selected from:

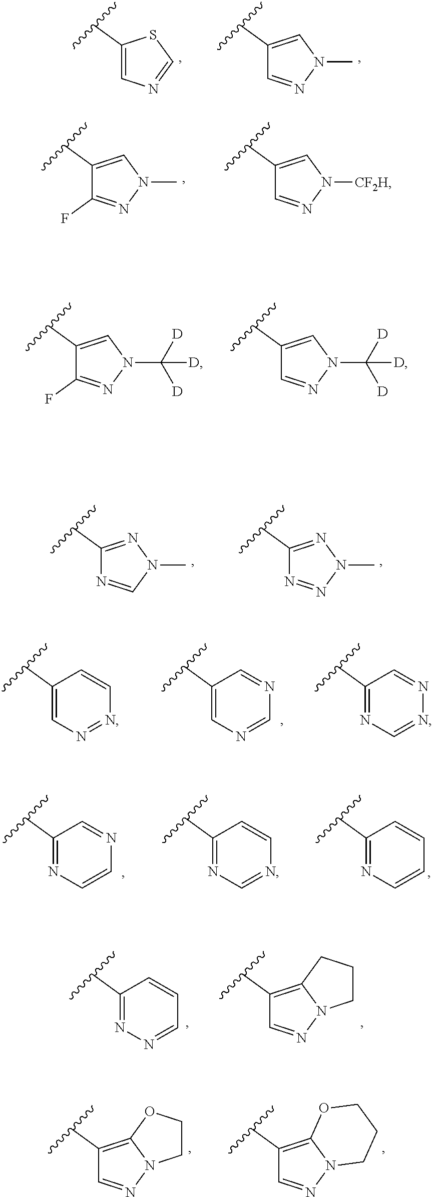

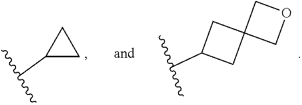

In some embodiments, R⁴ optionally substituted with one or more instances R^{4a} is selected from:

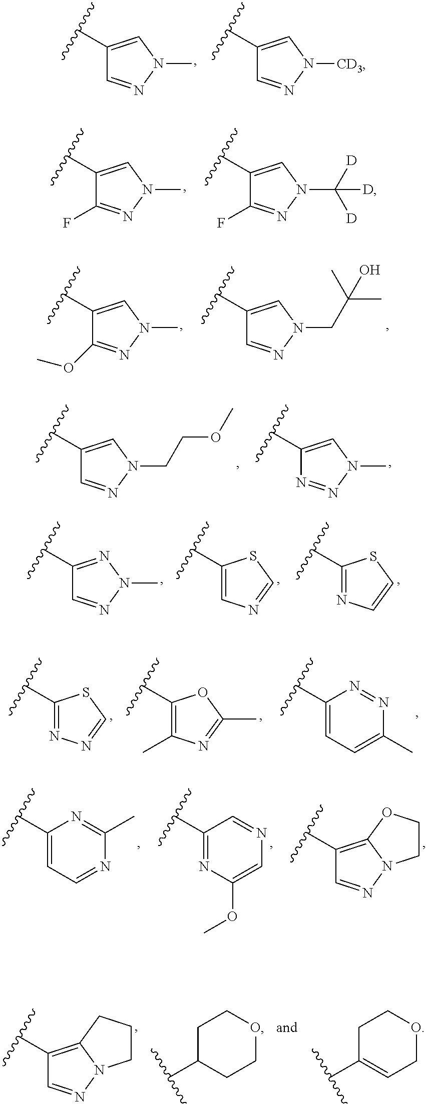

In some embodiments, R⁴ optionally substituted with one or more instances R^{4a} is selected from:

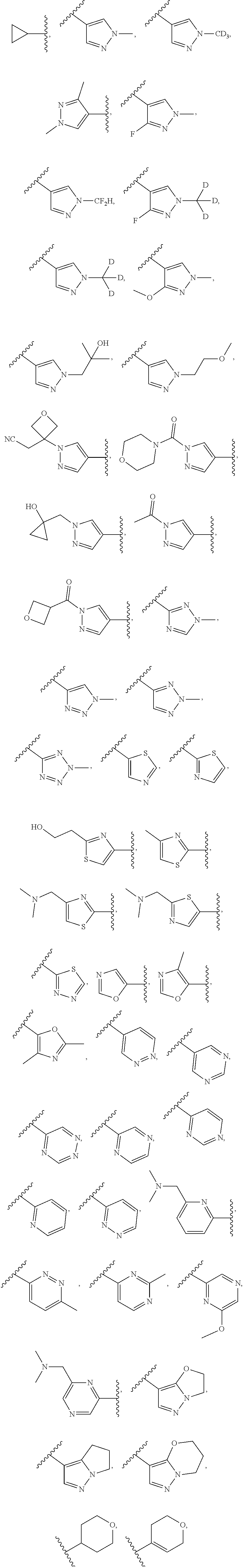

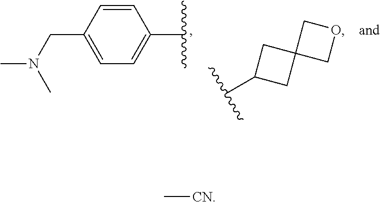

As described with respect to any formula provided herein, each R^{4a} is independently selected from optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)R^{a}, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}. In some embodiments, each R^{4a} is independently selected from optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}. In some embodiments, each R^{4a} is independently selected from optionally substituted C₁-C₆ aliphatic, —OR^{a}, and halogen. In some embodiments, each R^{4a} is independently selected from optionally substituted C₁-C₆ aliphatic, optionally substituted 2- to 10-membered heteroaliphatic, —OR^{a}, halogen, and —C(O)R^{a}. In some embodiments, each R^{4a} is independently selected from optionally substituted C₁-C₆ aliphatic and halogen. In some embodiments, each R^{4a} is independently selected from optionally substituted C₁-C₆ aliphatic, optionally substituted 2- to 10-membered heteroaliphatic, halogen, —C(O)R^{a}, and —OR^{a}.

In some embodiments, R^{4a} is optionally substituted C₁-C₆ aliphatic. In some embodiments, R^{4a} is optionally substituted C₁-C₆ alkyl. In some embodiments, R^{4a} is methyl or deuterated methyl (e.g., one or more H atoms of methyl have been replaced with deuterium). In some embodiments, R^{4a} is a branched C₁-C₆ aliphatic. In some embodiments, R^{4a} is C₁-C₆ aliphatic optionally substituted with halogen. In some embodiments, R^{4a} is C₁-C₆ aliphatic optionally substituted with one or more halogen or —OR^{∘}. In some embodiments, R^{4a} is C₁-C₆ aliphatic optionally substituted with one or more halogen, —CN, —OR^{∘}, or —(CH₂)₀₋₄R^{∘}. In some embodiments, R^{4a} is C₁-C₆ aliphatic optionally substituted with one or more halogen or —OR^{∘}, wherein each R^{∘} is independently C₁-C₆ alkyl or hydrogen. In some embodiments, R^{4a} is C₁-C₆ aliphatic optionally substituted with one or more halogen, —CN, —OR^{∘}, or —(CH₂)₀₋₄R^{∘}, wherein each R^{∘} is independently hydrogen, C₁₋₆ aliphatic, or two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur. In some embodiments, R^{4a} is —CH₃, —CD₃, —CH₂C(CH₃)₂OH, or —(CH₂)₂OCH₃. In some embodiments, R^{4a} is —CH₃, —CD₃, —CH₂CN, —CH₂C(CH₃)₂OH, —(CH₂)₂OH, —(CH₂)₂OCH₃,

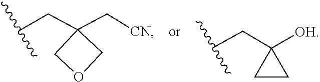 [SMILES: *CC1(CC#N)COC1 ; *CC1(O)CC1]

In some embodiments, R^{4a} is optionally substituted C₁-C₃ aliphatic.

In some embodiments, R^{4a} is optionally substituted 2- to 10-membered heteroaliphatic. In some embodiments, R^{4a} is methoxy. In some embodiments, R^{4a} is ethoxy. In some embodiments, R^{4a} is —CH₂N(CH₃)₂.

In some embodiments, R^{4a} is optionally substituted C₃-C₁₂ cycloaliphatic. In some embodiments, R^{4a} is optionally substituted C₃-C₆ cycloaliphatic. In some embodiments, R^{4a} is cyclopropyl.

In some embodiments, R^{4a} is optionally substituted 4- to 10-membered heterocycle comprising 1 to 4 heteroatoms selected from N, O, and S. In some embodiments, R^{4a} is 4- to 10-membered heterocycle comprising 1 to 4 heteroatoms selected from N, O, and S. In some embodiments, R^{4a} is optionally substituted 4- to 6-membered heterocycle comprising 1 to 4 heteroatoms selected from N, O, and S. In some embodiments, R^{4a} is a 4- to 6-membered heterocycle comprising 1 to 4 heteroatoms selected from N, O, and S. In some embodiments, R^{4a} is optionally substituted 4- to 6-membered heterocycle comprising 1 to 2 heteroatoms selected from N, O, and S. In some embodiments, R^{4a} is a 4- to 6-membered heterocycle comprising 1 to 2 heteroatoms selected from N, O, and S.

In some embodiments, R^{4a} is optionally substituted 5- to 10-membered heteroaryl comprising 1 to 4 heteroatoms selected from N, O, and S. In some embodiments, R^{4a} is optionally substituted 5- to 6-membered heteroaryl comprising 1 to 3 heteroatoms selected from N, O, and S.

In some embodiments, R^{4a} is optionally substituted C₆-C₁₀ aryl. In some embodiments, R^{4a} is optionally substituted phenyl. In some embodiments, R^{4a} is optionally substituted naphthyl.

In some embodiments, R^{4a} is halogen. In some embodiments, R^{4a} is fluoro. In some embodiments, R^{4a} is chloro.

In some embodiments, R^{4a} is oxo. In some embodiments, R^{4a} is —CN. In some embodiments, R^{4a} is —NO₂. In some embodiments, R^{4a} is —C(O)R^{a}. In some embodiments, R^{4a} is —C(O)(morpholinyl). In some embodiments, R^{4a} is —C(O)(CH₃). In some embodiments, R^{4a} is —C(O)(oxetanyl). In some embodiments, R^{4a} is —C(O)N(R^{a})₂. In some embodiments, R^{4a} is —C(O)NH₂. In some embodiments, R^{4a} is —C(O)N(CH₃)₂. In some embodiments, R^{4a} is —OC(O)R^{a}. In some embodiments, R^{4a} is —OC(O)CH₃. In some embodiments, R^{4a} is —OC(O)N(R^{a})₂. In some embodiments, R^{4a} is —OC(O)NH₂. In some embodiments, R^{4a} is —OC(O)N(CH₃)₂. In some embodiments, R^{4a} is —N(R^{a})C(O)R^{a}. In some embodiments, R^{4a} is —NHC(O)CH₃. In some embodiments, R^{4a} is —N(CH₃)C(O)CH₃. In some embodiments, R^{4a} is —N(R^{a})C(O)OR^{a}. In some embodiments, R^{4a} is —NHC(O)OH. In some embodiments, R^{4a} is —NHC(O)OCH₃. In some embodiments, R^{4a} is —N(R^{a})C(O)N(R^{a})₂. In some embodiments, R^{4a} is —NHC(O)NH₂. In some embodiments, R^{4a} is —NHC(O)N(CH₃)₂. In some embodiments, R^{4a} is —S(O)₂N(R^{a})₂. In some embodiments, R^{4a} is —S(O)₂NH₂. In some embodiments, R^{4a} is —S(O)₂N(CH₃)₂. In some embodiments, R^{4a} is —N(R^{a})S(O)₂R^{a}. In some embodiments, R^{4a} is —NHS(O)₂CH₃. In some embodiments, R^{4a} is-S(O)₂R^{a}. In some embodiments, R^{4a} is —OR^{a}. In some embodiments, R^{4a} is —OH. In some embodiments, R^{4a} is —OCH₃. In some embodiments, R^{4a} is —CH₃. In some embodiments, R^{4a} is —CD₃.

In some embodiments, each R^{4a} is independently selected from —CH₃, —CD₃, —CH₂C(CH₃)₂OH, —(CH₂)₂OCH₃, —OCH₃, and fluoro. In some embodiments, each R^{4a} is independently selected from —CH₃, —CD₃, —CH₂CN, —CH₂C(CH₃)₂OH, —(CH₂)₂OH, —(CH₂)₂OCH₃,

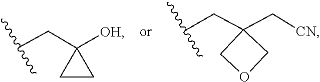 [SMILES: *CC1(O)CC1 ; *CC1(CC#N)COC1]

—OCH₃, —CH₂N(CH₃)₂, fluoro, —C(O)(CH₃), —C(O)(oxetanyl), and —C(O)(morpholinyl).

As described with respect to any formula provided herein (e.g., any of Formulae I-IX), each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S. In some embodiments, each R^{a} is independently selected from hydrogen and optionally substituted C₁-C₆ aliphatic (e.g., optionally substituted C₁-C₆ alkyl). In some embodiments, each R^{a} is independently selected from hydrogen and C₁-C₆ aliphatic. In some embodiments, each R^{a} is independently selected from hydrogen and C₁-C₆ alkyl. In some embodiments, each R^{a} is independently selected from hydrogen, optionally substituted C₁-C₆ aliphatic (e.g., optionally substituted C₁-C₆ alkyl), and an optionally substituted 4- to 12-membered heterocycle comprising 1 to 4 heteroatoms selected from N, O, and S. In some embodiments, each R^{a} is independently selected from hydrogen, C₁-C₆ aliphatic, and a 4- to 6-membered heterocycle comprising 1 to 4 heteroatoms selected from N, O, and S. In some embodiments, each R^{a} is independently selected from hydrogen, C₁-C₆ alkyl, oxetanyl, and morpholinyl.

In some embodiments, R^{a} is hydrogen.

In some embodiments, R^{a} is optionally substituted C₁-C₆ aliphatic. In some embodiments, R^{a} is C₁-C₆ aliphatic. In some embodiments, R^{a} is optionally substituted C₁-C₆ alkyl. In some embodiments, R^{a} is C₁-C₆ alkyl. In some embodiments, R^{a} is methyl or ethyl.

In some embodiments, R^{a} is optionally substituted 2- to 10-membered heteroaliphatic.

In some embodiments, R^{a} is optionally substituted 5- to 12-membered heteroaryl comprising 1 to 4 heteroatoms selected from N, O, and S. In some embodiments, R^{a} is optionally substituted 5- to 6-membered heteroaryl comprising 1 to 3 heteroatoms selected from N, O, and S.

In some embodiments, R^{a} is optionally substituted 4- to 12-membered heterocycle comprising 1 to 4 heteroatoms selected from N, O, and S. In some embodiments, R^{a} is optionally substituted 4- to 6-membered heterocycle comprising 1 to 2 heteroatoms selected from N, O, and S. In some embodiments, R^{a} is a 4- to 6-membered heterocycle comprising 1 to 2 heteroatoms selected from N, O, and S. In some embodiments, R^{a} is oxetanyl. In some embodiments, R^{a} is morpholinyl.

In some embodiments, the present disclosure provides a compound of Formula I:

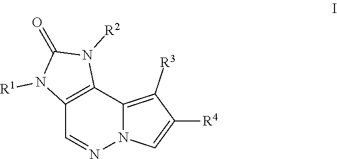

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is optionally substituted with one or more instances of -L-R⁵;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a}), —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a};
R⁴ is C₁-C₆ aliphatic, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, the present disclosure provides a compound of Formula I:

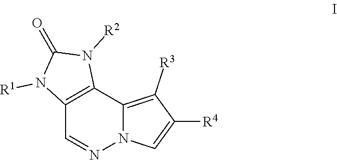

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is optionally substituted with one or more instances of -L-R⁵;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})_, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a};
R⁴ is C₁-C₆ aliphatic, —CN, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)R^{a}, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, the present disclosure provides a compound of Formula I:

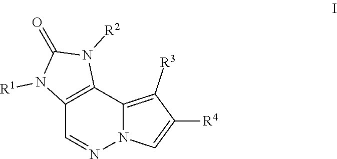

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is optionally substituted with one or more instances of -L-R⁵ or R⁷;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a}), —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
each R⁷ is independently selected from the group consisting of halogen, —OH, —SH, and —NH₂;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a};
R⁴ is C₁-C₆ aliphatic, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S. In some embodiments, the present disclosure provides a compound of Formula I:

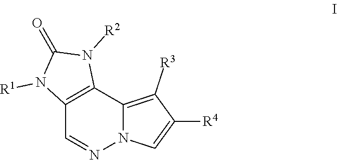

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is optionally substituted with one or more instances of -L-R⁵ or R⁷;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a}), —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
each R⁷ is independently selected from the group consisting of halogen, —OH, —SH, and —NH₂;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a};
R⁴ is C₁-C₆ aliphatic, —CN, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)R^{a}, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, the present disclosure provides a compound of Formula VIII:

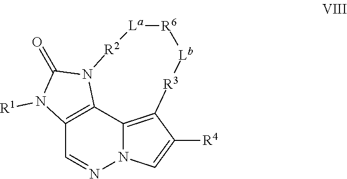

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a};
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
L^{b} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
R⁶ is optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, or optionally substituted C₆-C₁₀ aryl;
R⁴ is C₁-C₆ aliphatic, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, the present disclosure provides a compound of Formula VIII:

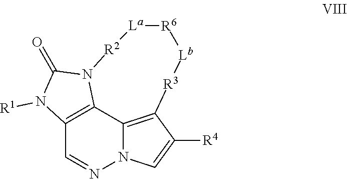

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a};
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
L^{b} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
R⁶ is optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, or optionally substituted C₆-C₁₀ aryl;
R⁴ is C₁-C₆ aliphatic, —CN, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)R^{a}, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, the present disclosure provides a compound of Formula I:

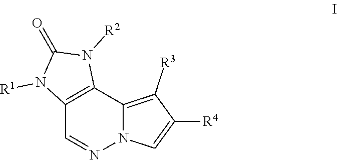

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is substituted with one or more instances of -L-R⁵ or R⁷;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a}—), —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
each R⁷ is independently selected from the group consisting of halogen, —OH, —SH, and —NH₂;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a}; or
R² and R³ are connected by *-L^{a}-R⁶-L^{b}-**, wherein * represents a point of attachment to R², and ** represents a point of attachment to R³;
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
L^{b} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
R⁶ is optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, or optionally substituted C₆-C₁₀ aryl;
R⁴ is C₁-C₆ aliphatic, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, the present disclosure provides a compound of Formula I:

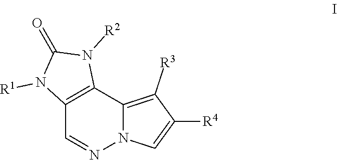

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is substituted with one or more instances of -L-R⁵ or R⁷;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a}—), —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
each R⁷ is independently selected from the group consisting of halogen, —OH, —SH, and —NH₂;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a}; or
R² and R³ are connected by *-L^{a}-R⁶-L^{b}-**, wherein * represents a point of attachment to R², and ** represents a point of attachment to R³;
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
L^{b} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
R⁶ is optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, or optionally substituted C₆-C₁₀ aryl;
R⁴ is C₁-C₆ aliphatic, —CN, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)R^{a}, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, the present disclosure provides a compound of Formula I:

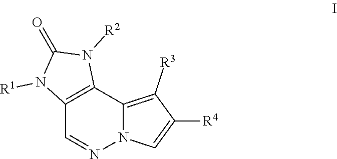

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, or 5- to 10-membered heteroaryl comprising 1 to 4 heteroatoms selected from N, O, and S, wherein R² is optionally substituted with one instance of -L-R⁵;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a}), —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, or C₃-C₁₂ cycloaliphatic, wherein R³ is optionally substituted with one or more instances of R^{3a}; or
R² and R³ are connected by *-L^{a}-R⁶-L^{b}-**, where * represents a point of attachment to R², and ** represents a point of attachment to R³;
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
L^{b} is a bond, —C(O)N(R^{a})—, —N(R^{a})C(O)—, —N(R^{a})—, —O—, —C(O)—, —C(O)O—, —OC(O)—, —C(S)N(R^{a})—, —N(R^{a})C(S)—, —C(S)—, —C(S)O—, or —OC(S)—;
R⁶ is optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, or optionally substituted 5- to 10-membered heteroaryl comprising 1 to 4 heteroatoms selected from N, O, and S;
R⁴ is C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, halogen, oxo, —CN, —NO₂, —C(O)N(R^{a})₂, —OC(O)R^{a}, OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, the present disclosure provides a compound of Formula I:

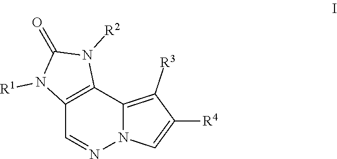

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, or 5- to 10-membered heteroaryl comprising 1 to 4 heteroatoms selected from N, O, and S, wherein R² is optionally substituted with one instance of -L-R⁵;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a}), —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, or C₃-C₁₂ cycloaliphatic, wherein R³ is optionally substituted with one or more instances of R^{3a}; or
R² and R³ are connected by *-L^{a}-R⁶-L^{b}-**, where * represents a point of attachment to R², and ** represents a point of attachment to R³;
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
L^{b} is a bond, —C(O)N(R^{a})—, —N(R^{a})C(O)—, —N(R^{a})—, —O—, —C(O)—, —C(O)O—, —OC(O)—, —C(S)N(R^{a})—, —N(R^{a})C(S)—, —C(S)—, —C(S)O—, or —OC(S)—;
R⁶ is optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, or optionally substituted 5- to 10-membered heteroaryl comprising 1 to 4 heteroatoms selected from N, O, and S;
R⁴ is —CN, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, halogen, oxo, —CN, —NO₂, —C(O)N(R^{a})₂, —OC(O)R^{a}, OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, the present disclosure provides a compound of Formula I:

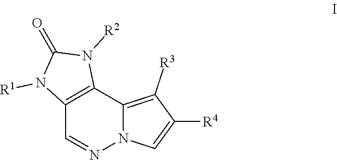

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, or 4- to 10-membered heterocycle comprising 1 to 4 heteroatoms selected from N, O, and S, wherein R² is optionally substituted with one instance of -L-R⁵;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a}), —O—, —C(O)—, —C(O)O—, and —OC(O)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, and optionally substituted 4- to 10-membered heterocycle comprising 1 to 4 heteroatoms selected from N, O, and S;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a}; or
R² and R³ are connected by *-L^{a}-R⁶-L^{b}-**, where * represents a point of attachment to R², and ** represents a point of attachment to R³;
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, or —OC(O)—;
L^{b} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, or —OC(O)—;
R⁶ is optionally substituted C₁-C₆ aliphatic, optionally substituted 2- to 10-membered heteroaliphatic, optionally substituted C₃-C₁₂ cycloaliphatic, or optionally substituted 4- to 10-membered heterocycle comprising 1 to 4 heteroatoms selected from N, O, and S;
R⁴ is C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, halogen, oxo, —CN, —NO₂, —C(O)N(R^{a})₂, —OC(O)R^{a}, OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, halogen, oxo, —CN, —NO₂, —C(O)N(R^{a})₂, —OC(O)R^{a}, OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, the present disclosure provides a compound of Formula I:

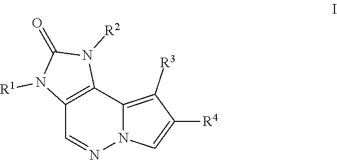

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, or 4- to 10-membered heterocycle comprising 1 to 4 heteroatoms selected from N, O, and S, wherein R² is optionally substituted with one instance of -L-R⁵;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})_, —O—, —C(O)—, —C(O)O—, and —OC(O)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, and optionally substituted 4- to 10-membered heterocycle comprising 1 to 4 heteroatoms selected from N, O, and S;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a}; or
R² and R³ are connected by *-L^{a}-R⁶-L^{b}-**, where * represents a point of attachment to R², and ** represents a point of attachment to R³;
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, or —OC(O)—;
L^{b} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, or —OC(O)—;
R⁶ is optionally substituted C₁-C₆ aliphatic, optionally substituted 2- to 10-membered heteroaliphatic, optionally substituted C₃-C₁₂ cycloaliphatic, or optionally substituted 4- to 10-membered heterocycle comprising 1 to 4 heteroatoms selected from N, O, and S;
R⁴ is —CN, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, halogen, oxo, —CN, —NO₂, —C(O)N(R^{a})₂, —OC(O)R^{a}, OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, halogen, oxo, —CN, —NO₂, —C(O)R^{a}, —C(O)N(R^{a})₂, —OC(O)R^{a}, OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

In some embodiments, the present disclosure provides a compound selected from Table 1A, or a pharmaceutically acceptable salt thereof. [REMAINDER OF THIS PAGE INTENTIONALLY LEFT BLANK]

TABLE 1A

| No. | Structure |
|---|---|
| I-1 | 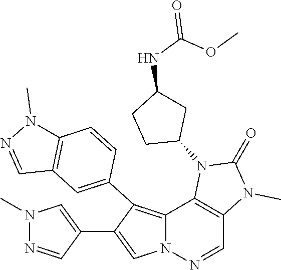 |
| I-1′ | 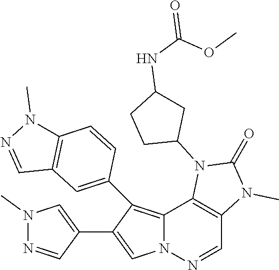 |
| I-2 | 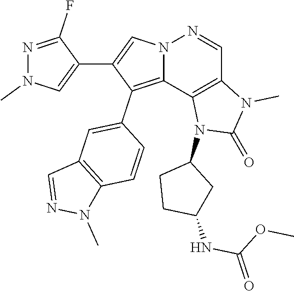 |
| I-2′ | 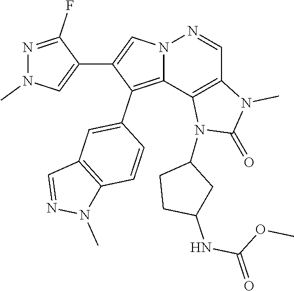 |
| I-3 | 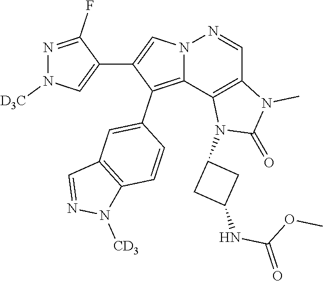 [SMILES: [2H]C([2H])([2H])n1cc(-c2cn3ncc4c(c3c2-c2ccc3c(cnn3C([2H])([2H])[2H])c2)n([C@H]2C[C@@H](NC(=O)OC)C2)c(=O)n4C)c(F)n1] |
| I-3′ | 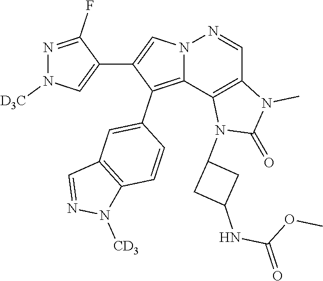 [SMILES: [2H]C([2H])([2H])n1cc(-c2cn3ncc4c(c3c2-c2ccc3c(cnn3C([2H])([2H])[2H])c2)n(C2CC(NC(=O)OC)C2)c(=O)n4C)c(F)n1] |
| I-4-1 | 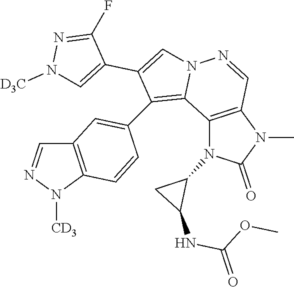 [SMILES: [2H]C([2H])([2H])n1cc(-c2cn3ncc4c(c3c2-c2ccc3c(cnn3C([2H])([2H])[2H])c2)n([C@H]2C[C@@H]2NC(=O)OC)c(=O)n4C)c(F)n1] |
| I-4-2 | 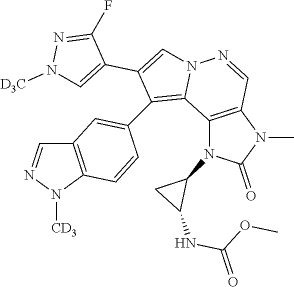 [SMILES: [2H]C([2H])([2H])n1cc(-c2cn3ncc4c(c3c2-c2ccc3c(cnn3C([2H])([2H])[2H])c2)n([C@@H]2C[C@H]2NC(=O)OC)c(=O)n4C)c(F)n1] |
| I-4′ | 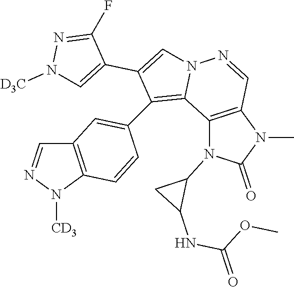 [SMILES: [2H]C([2H])([2H])n1cc(-c2cn3ncc4c(c3c2-c2ccc3c(cnn3C([2H])([2H])[2H])c2)n(C2CC2NC(=O)OC)c(=O)n4C)c(F)n1] |
| I-5 | 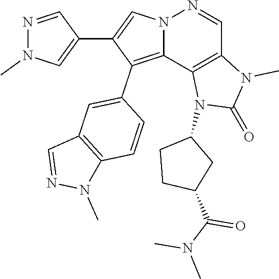 |
| I-5′ | 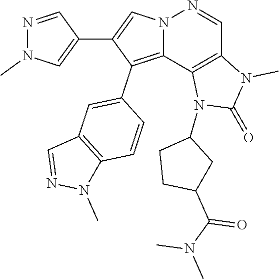 |
| I-6 | 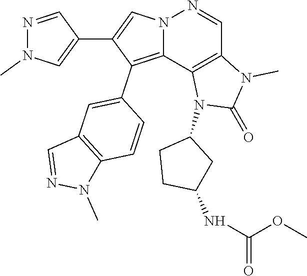 |
| I-6′ | 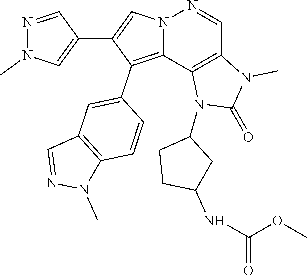 |
| I-7 | 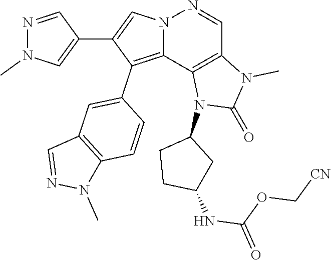 [SMILES: Cn1cc(-c2cn3ncc4c(c3c2-c2ccc3c(cnn3C)c2)n([C@@H]2CC[C@@H](NC(=O)OCC#N)C2)c(=O)n4C)cn1] |
| I-7′ | 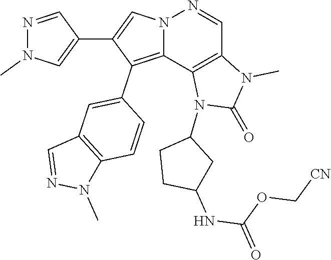 [SMILES: Cn1cc(-c2cn3ncc4c(c3c2-c2ccc3c(cnn3C)c2)n(C2CCC(NC(=O)OCC#N)C2)c(=O)n4C)cn1] |
| I-8 | 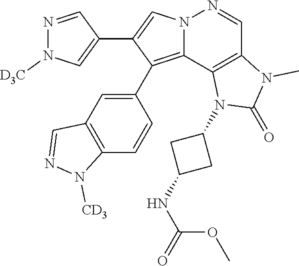 [SMILES: [2H]C([2H])([2H])n1cc(-c2cn3ncc4c(c3c2-c2ccc3c(cnn3C([2H])([2H])[2H])c2)n([C@H]2C[C@@H](NC(=O)OC)C2)c(=O)n4C)cn1] |
| I-8′ | 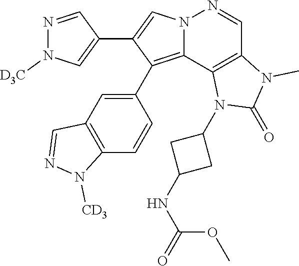 [SMILES: [2H]C([2H])([2H])n1cc(-c2cn3ncc4c(c3c2-c2ccc3c(cnn3C([2H])([2H])[2H])c2)n(C2CC(NC(=O)OC)C2)c(=O)n4C)cn1] |
| I-9 | 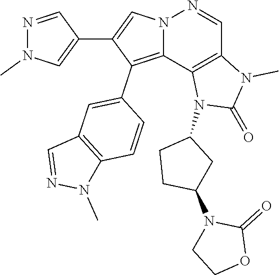 |
| I-9′ | 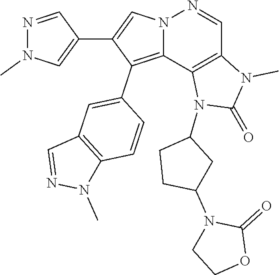 |
| I-10 | 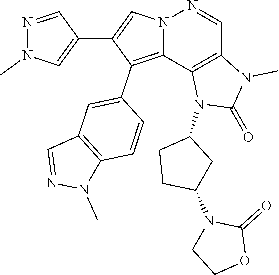 |
| I-10′ | 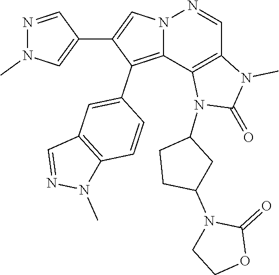 |
| I-11 | 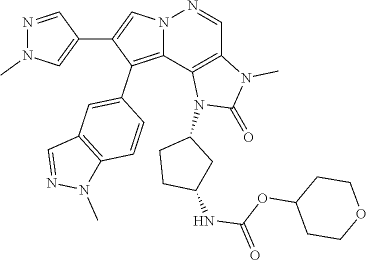 [SMILES: Cn1cc(-c2cn3ncc4c(c3c2-c2ccc3c(cnn3C)c2)n([C@H]2CC[C@@H](NC(=O)OC3CCOCC3)C2)c(=O)n4C)cn1] |
| I-11′ | 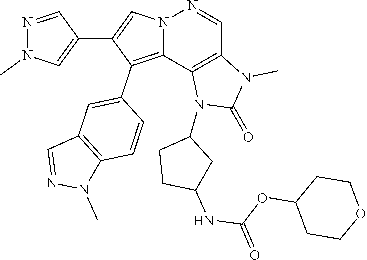 [SMILES: Cn1cc(-c2cn3ncc4c(c3c2-c2ccc3c(cnn3C)c2)n(C2CCC(NC(=O)OC3CCOCC3)C2)c(=O)n4C)cn1] |
| I-12 | 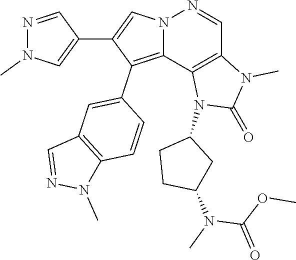 |
| I-12′ | 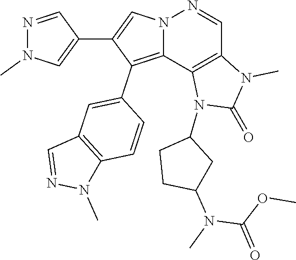 |
| I-13 | 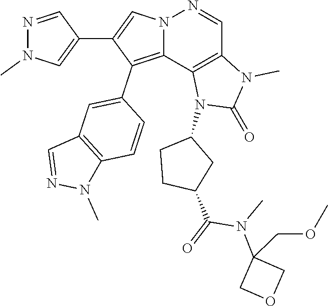 |
| I-13′ | 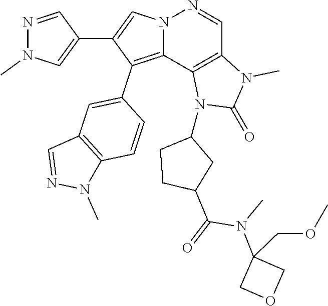 |
| I-14 | 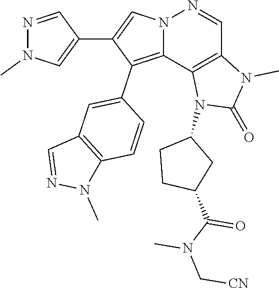 |
| I-14′ | 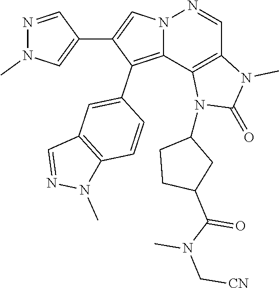 |
| I-15 | 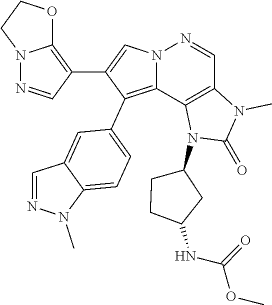 |
| I-15′ | 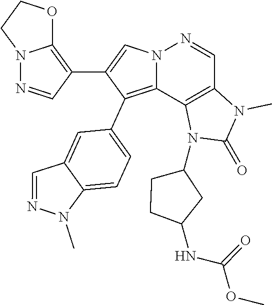 [SMILES: COC(=O)NC1CCC(n2c(=O)n(C)c3cnn4cc(-c5cnn6c5OCC6)c(-c5ccc6c(cnn6C)c5)c4c32)C1] |
| I-16 | 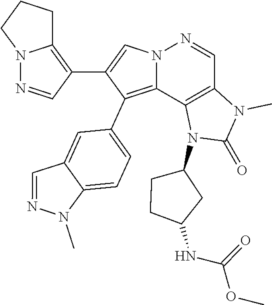 |
| I-16′ | 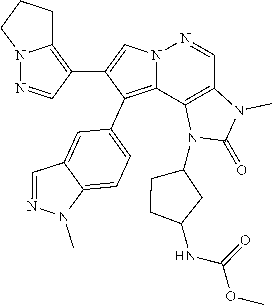 [SMILES: COC(=O)NC1CCC(n2c(=O)n(C)c3cnn4cc(-c5cnn6c5CCC6)c(-c5ccc6c(cnn6C)c5)c4c32)C1] |
| I-17 | 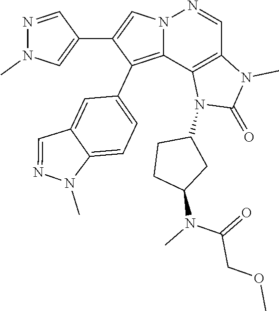 |
| I-17′ | 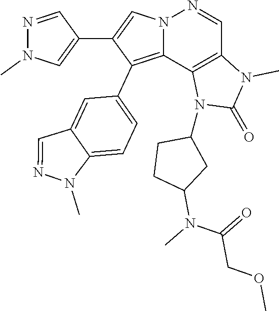 |
| I-18 | 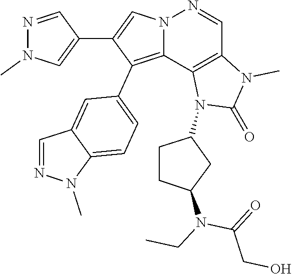 |
| I-18′ | 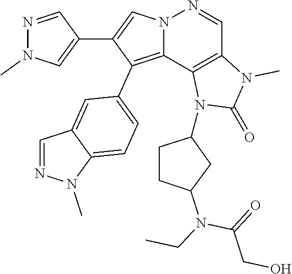 |
| I-19 | 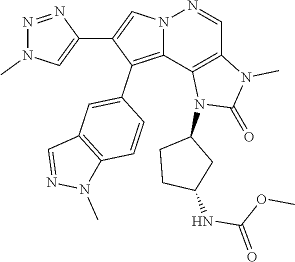 |
| I-19′ | 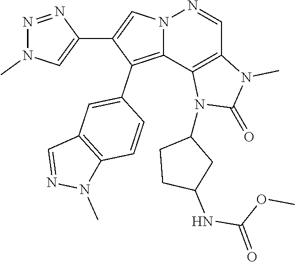 |
| I-20-1 | 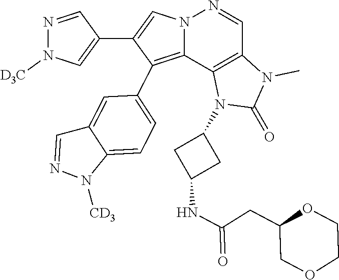 [SMILES: [2H]C([2H])([2H])n1cc(-c2cn3ncc4c(c3c2-c2ccc3c(cnn3C([2H])([2H])[2H])c2)n([C@H]2C[C@@H](NC(=O)C[C@@H]3COCCO3)C2)c(=O)n4C)cn1] |
| I-20-2 | 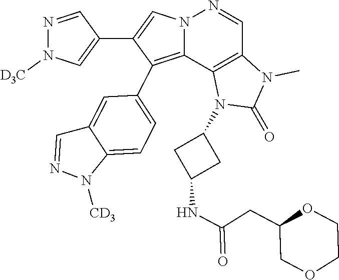 [SMILES: [2H]C([2H])([2H])n1cc(-c2cn3ncc4c(c3c2-c2ccc3c(cnn3C([2H])([2H])[2H])c2)n([C@H]2C[C@@H](NC(=O)C[C@@H]3COCCO3)C2)c(=O)n4C)cn1] |
| I-20′ | 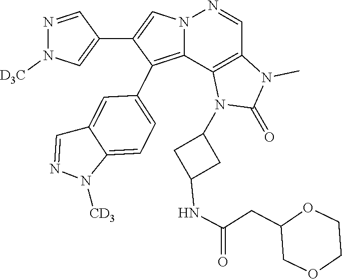 [SMILES: [2H]C([2H])([2H])n1cc(-c2cn3ncc4c(c3c2-c2ccc3c(cnn3C([2H])([2H])[2H])c2)n(C2CC(NC(=O)CC3COCCO3)C2)c(=O)n4C)cn1] |
| I-21 | 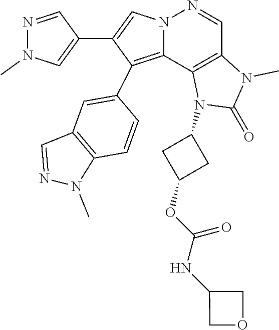 |
| I-21′ | 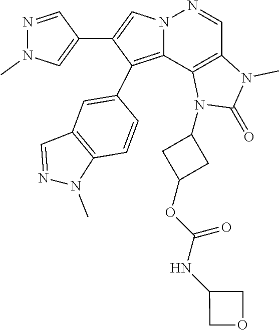 |
| I-22 | 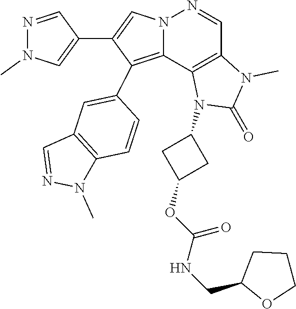 [SMILES: Cn1cc(-c2cn3ncc4c(c3c2-c2ccc3c(cnn3C)c2)n([C@H]2C[C@@H](OC(=O)NC[C@H]3CCCO3)C2)c(=O)n4C)cn1] |
| I-22′ | 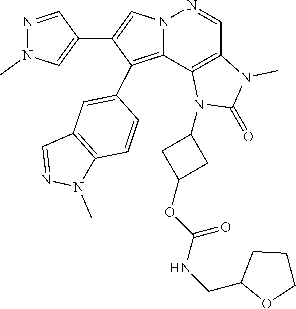 [SMILES: Cn1cc(-c2cn3ncc4c(c3c2-c2ccc3c(cnn3C)c2)n(C2CC(OC(=O)NCC3CCCO3)C2)c(=O)n4C)cn1] |
| I-23 | 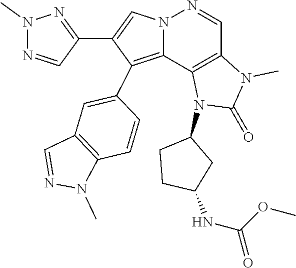 |
| I-23′ | 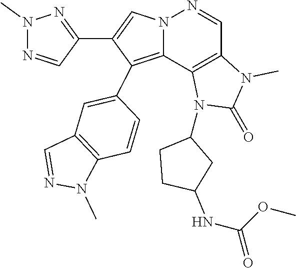 |
| I-24 | 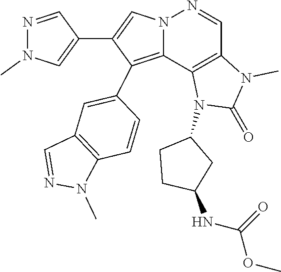 |
| I-24′ | 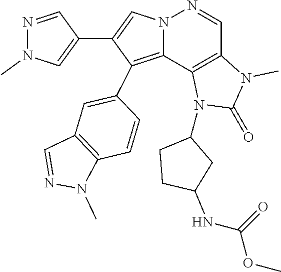 |
| I-25 | 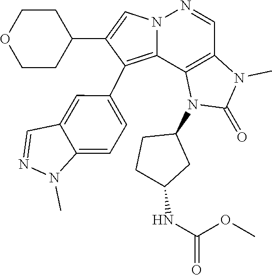 [SMILES: COC(=O)N[C@@H]1CC[C@@H](n2c(=O)n(C)c3cnn4cc(C5CCOCC5)c(-c5ccc6c(cnn6C)c5)c4c32)C1] |
| I-25′ | 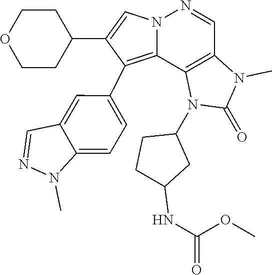 [SMILES: COC(=O)NC1CCC(n2c(=O)n(C)c3cnn4cc(C5CCOCC5)c(-c5ccc6c(cnn6C)c5)c4c32)C1] |
| I-26 | 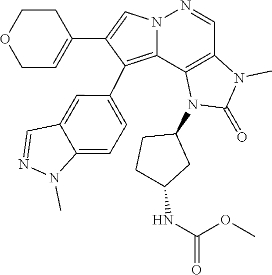 [SMILES: COC(=O)N[C@@H]1CC[C@@H](n2c(=O)n(C)c3cnn4cc(C5=CCOCC5)c(-c5ccc6c(cnn6C)c5)c4c32)C1] |
| I-26′ | 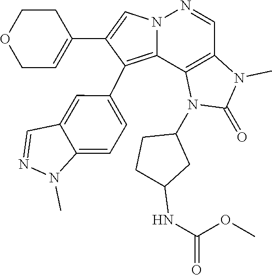 [SMILES: COC(=O)NC1CCC(n2c(=O)n(C)c3cnn4cc(C5=CCOCC5)c(-c5ccc6c(cnn6C)c5)c4c32)C1] |
| I-27 | 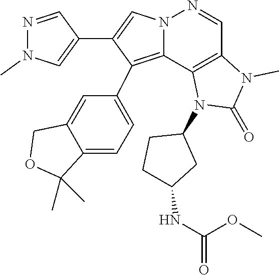 |
| I-27′ | 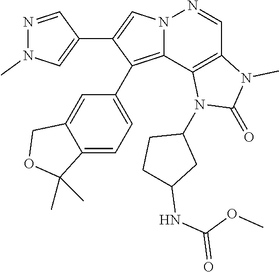 |
| I-28 | 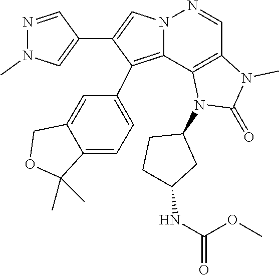 |
| I-28′ | 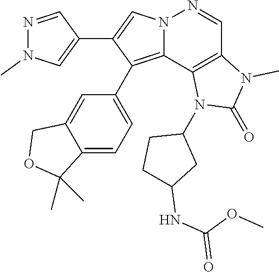 |
| I-29 | 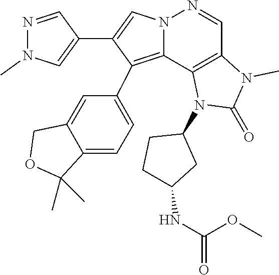 |
| I-29′ | 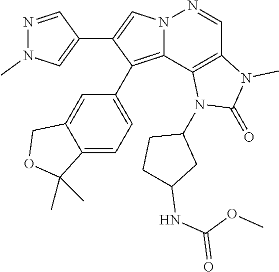 |
| I-30 | 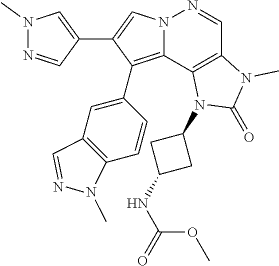 |
| I-30′ | 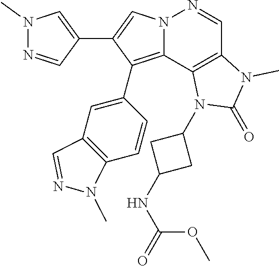 |
| I-31 | 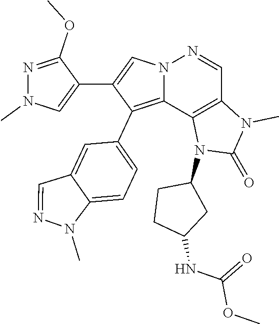 |
| I-31′ | 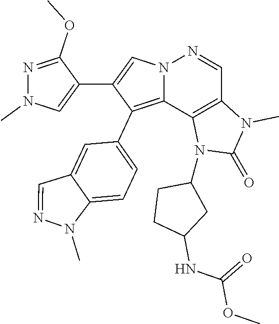 |
| I-32 | 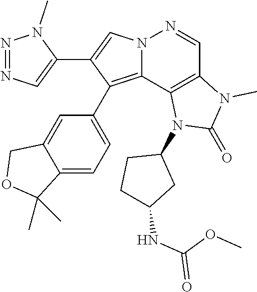 [SMILES: COC(=O)N[C@@H]1CC[C@@H](n2c(=O)n(C)c3cnn4cc(-c5cnnn5C)c(-c5ccc6c(c5)COC6(C)C)c4c32)C1] |
| I-32′ | 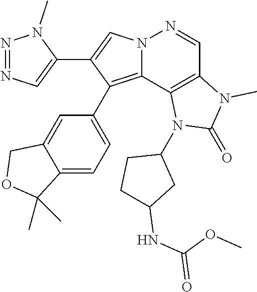 [SMILES: COC(=O)NC1CCC(n2c(=O)n(C)c3cnn4cc(-c5cnnn5C)c(-c5ccc6c(c5)COC6(C)C)c4c32)C1] |
| I-33 | 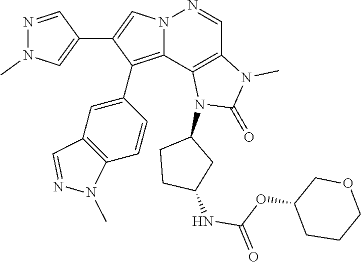 [SMILES: Cn1cc(-c2cn3ncc4c(c3c2-c2ccc3c(cnn3C)c2)n([C@@H]2CC[C@@H](NC(=O)O[C@H]3CCCOC3)C2)c(=O)n4C)cn1] |
| I-33′ | 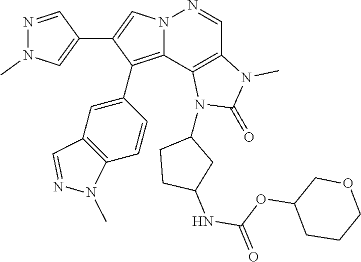 [SMILES: Cn1cc(-c2cn3ncc4c(c3c2-c2ccc3c(cnn3C)c2)n(C2CCC(NC(=O)OC3CCCOC3)C2)c(=O)n4C)cn1] |
| I-34 | 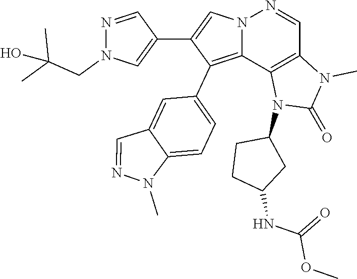 |
| I-34′ | 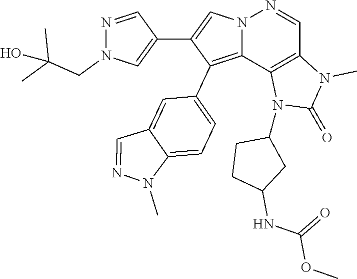 [SMILES: COC(=O)NC1CCC(n2c(=O)n(C)c3cnn4cc(-c5cnn(CC(C)(C)O)c5)c(-c5ccc6c(cnn6C)c5)c4c32)C1] |
| I-35 | 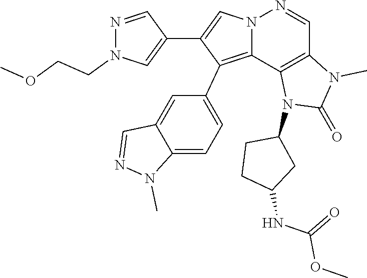 |
| I-35′ | 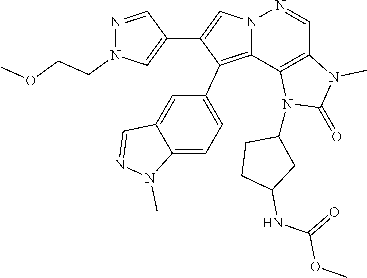 |
| I-36 | 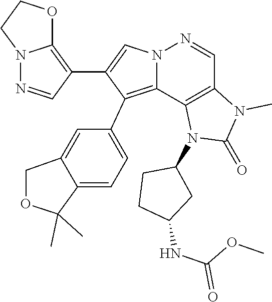 [SMILES: COC(=O)N[C@@H]1CC[C@@H](n2c(=O)n(C)c3cnn4cc(-c5cnn6c5OCC6)c(-c5ccc6c(c5)COC6(C)C)c4c32)C1] |
| I-36′ | 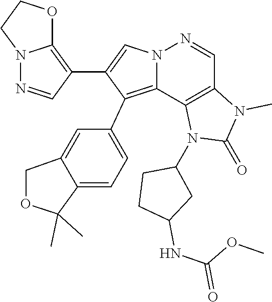 [SMILES: COC(=O)NC1CCC(n2c(=O)n(C)c3cnn4cc(-c5cnn6c5OCC6)c(-c5ccc6c(c5)COC6(C)C)c4c32)C1] |
| I-37-1 | 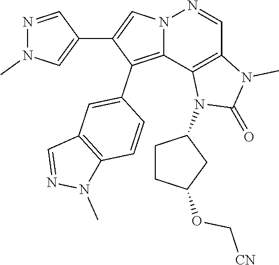 |
| I-37-2 | 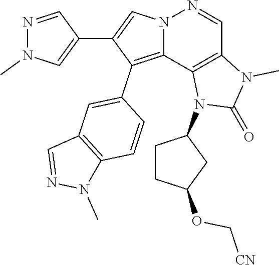 |
| I-37′ | 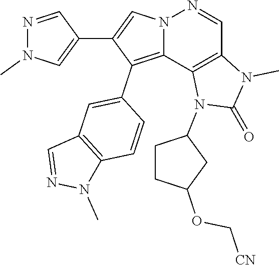 |
| I-38 | 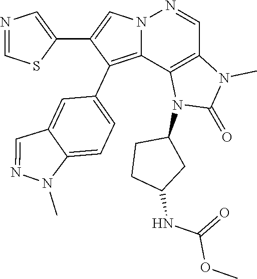 |
| I-38′ | 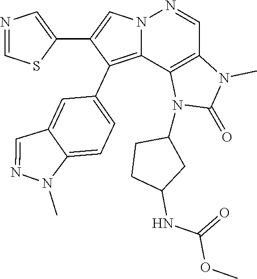 [SMILES: COC(=O)NC1CCC(n2c(=O)n(C)c3cnn4cc(-c5cncs5)c(-c5ccc6c(cnn6C)c5)c4c32)C1] |
| I-40 | 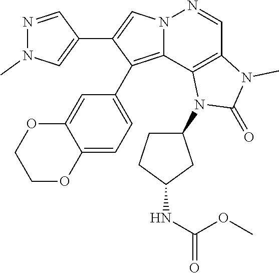 |
| I-40′ | 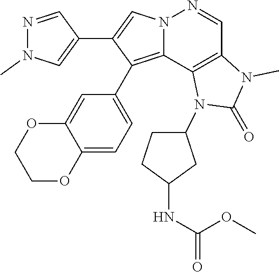 |
| I-41 | 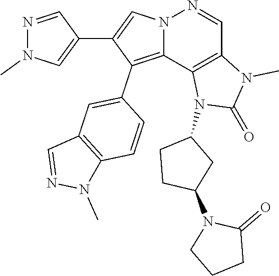 |
| I-41′ | 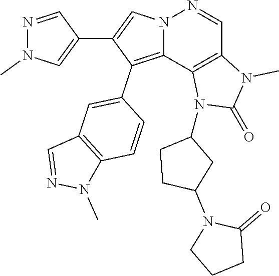 |
| I-42 | 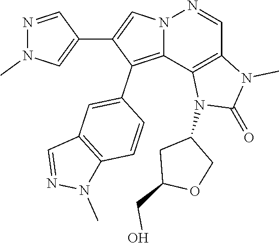 |
| I-42′ | 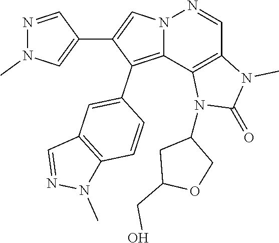 |
| I-43 | 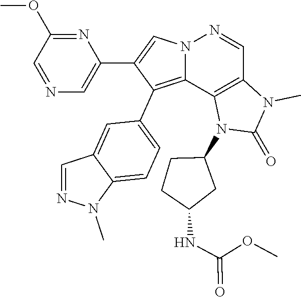 |
| I-43′ | 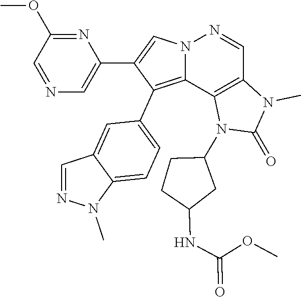 |
| I-44 | 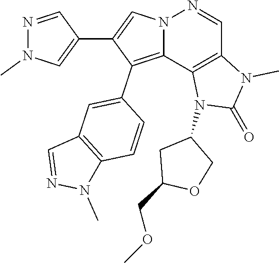 |
| I-44′ | 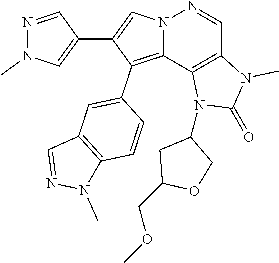 |
| I-45 | 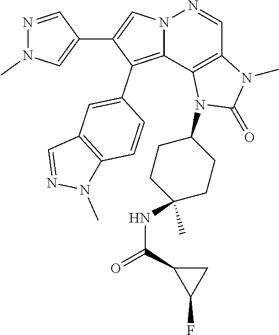 |
| I-45′ | 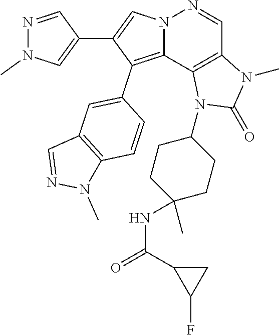 |
| I-46 | 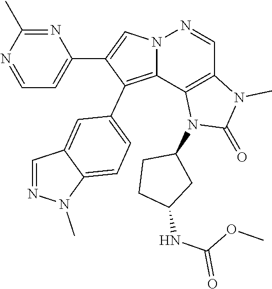 [SMILES: COC(=O)N[C@@H]1CC[C@@H](n2c(=O)n(C)c3cnn4cc(-c5ccnc(C)n5)c(-c5ccc6c(cnn6C)c5)c4c32)C1] |
| I-46′ | 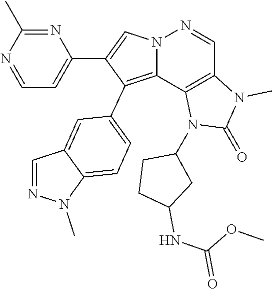 [SMILES: COC(=O)NC1CCC(n2c(=O)n(C)c3cnn4cc(-c5ccnc(C)n5)c(-c5ccc6c(cnn6C)c5)c4c32)C1] |
| I-47 | 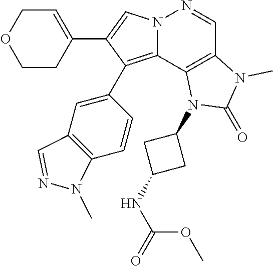 [SMILES: COC(=O)N[C@H]1C[C@H](n2c(=O)n(C)c3cnn4cc(C5=CCOCC5)c(-c5ccc6c(cnn6C)c5)c4c32)C1] |
| I-47′ | 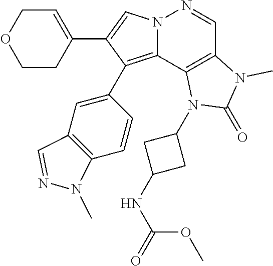 [SMILES: COC(=O)NC1CC(n2c(=O)n(C)c3cnn4cc(C5=CCOCC5)c(-c5ccc6c(cnn6C)c5)c4c32)C1] |
| I-48 | 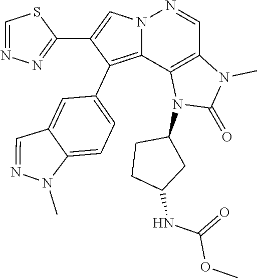 |
| I-48′ | 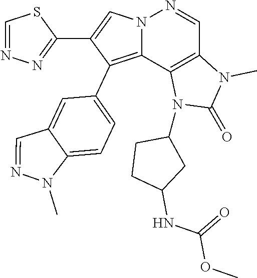 [SMILES: COC(=O)NC1CCC(n2c(=O)n(C)c3cnn4cc(-c5nncs5)c(-c5ccc6c(cnn6C)c5)c4c32)C1] |
| I-49 | 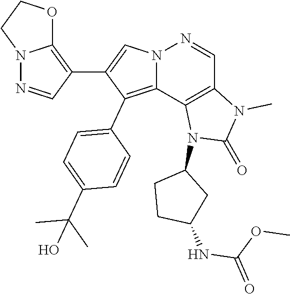 [SMILES: COC(=O)N[C@@H]1CC[C@@H](n2c(=O)n(C)c3cnn4cc(-c5cnn6c5OCC6)c(-c5ccc(C(C)(C)O)cc5)c4c32)C1] |
| I-49′ | 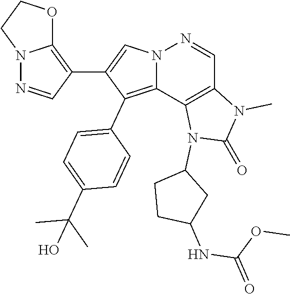 [SMILES: COC(=O)NC1CCC(n2c(=O)n(C)c3cnn4cc(-c5cnn6c5OCC6)c(-c5ccc(C(C)(C)O)cc5)c4c32)C1] |
| I-50 | 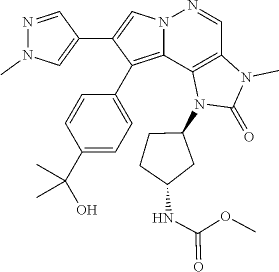 |
| I-50′ | 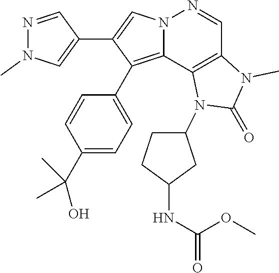 |
| I-51 | 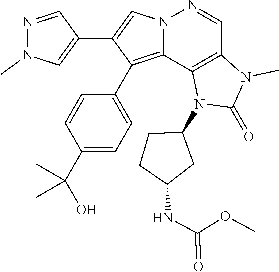 |
| I-51′ | 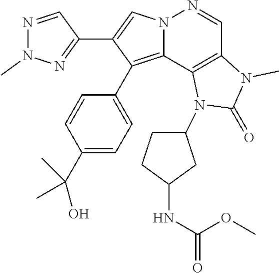 |
| I-52 | 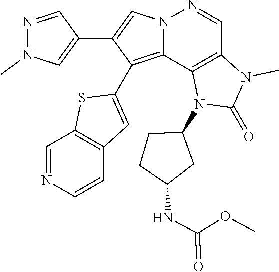 |
| I-52′ | 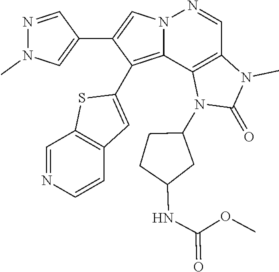 |
| I-53 | 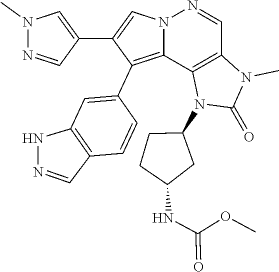 |
| I-53′ | 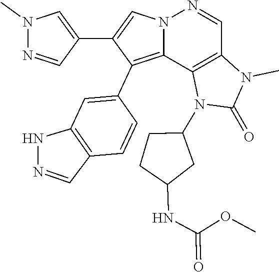 |
| I-54 | 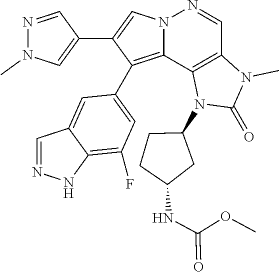 |
| I-54′ | 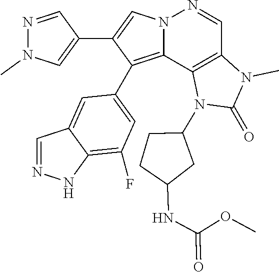 |
| I-55 | 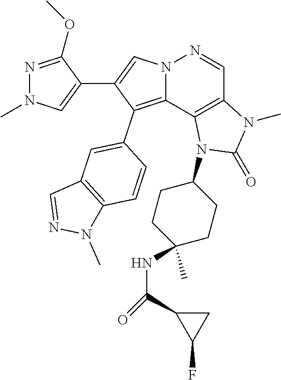 |
| I-55′ | 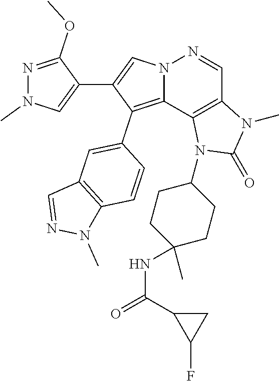 |
| I-56 | 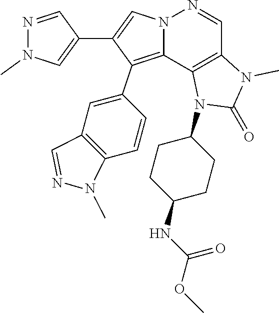 |
| I-56′ | 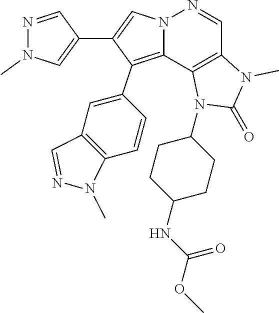 |
| I-57 | 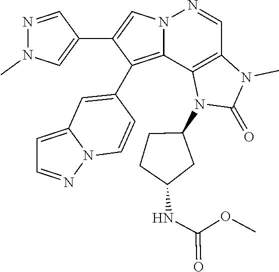 |
| I-57′ | 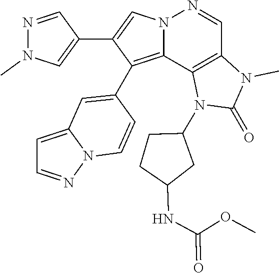 |
| I-58 | 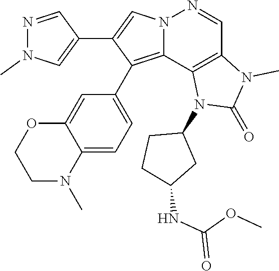 |
| I-58′ | 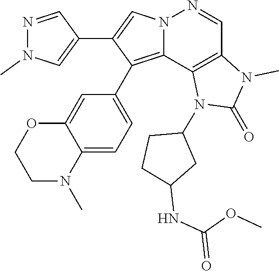 |
| I-59 | 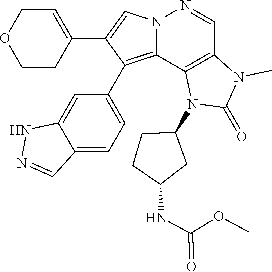 [SMILES: COC(=O)N[C@@H]1CC[C@@H](n2c(=O)n(C)c3cnn4cc(C5=CCOCC5)c(-c5ccc6cn[nH]c6c5)c4c32)C1] |
| I-59′ | 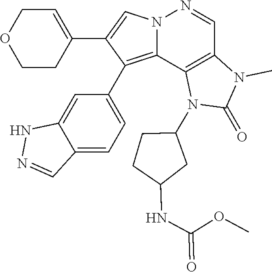 [SMILES: COC(=O)NC1CCC(n2c(=O)n(C)c3cnn4cc(C5=CCOCC5)c(-c5ccc6cn[nH]c6c5)c4c32)C1] |
| I-60 | 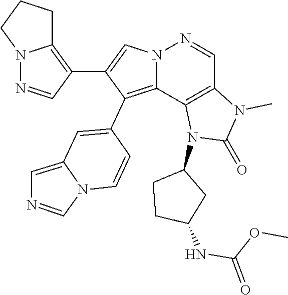 [SMILES: COC(=O)N[C@@H]1CC[C@@H](n2c(=O)n(C)c3cnn4cc(-c5cnn6c5CCC6)c(-c5ccn6cncc6c5)c4c32)C1] |
| I-60′ | 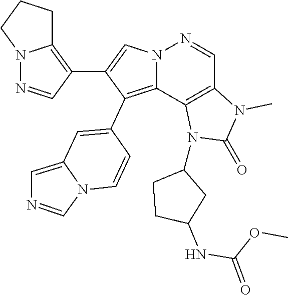 [SMILES: COC(=O)NC1CCC(n2c(=O)n(C)c3cnn4cc(-c5cnn6c5CCC6)c(-c5ccn6cncc6c5)c4c32)C1] |
| I-61 | 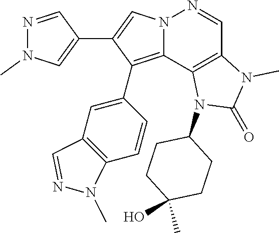 |
| I-61′ | 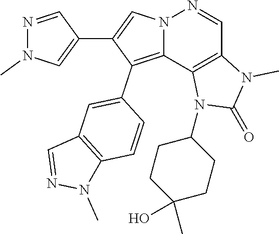 |
| I-62 | 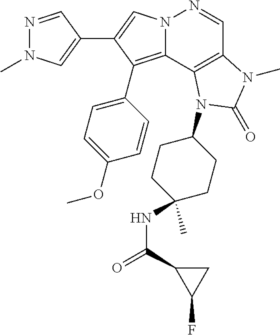 |
| I-62′ | 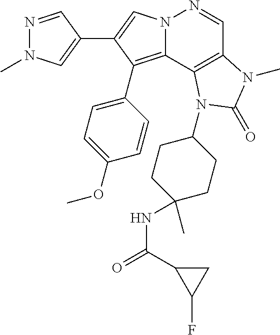 |
| I-63 | 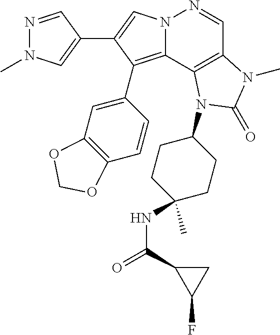 |
| I-63′ | 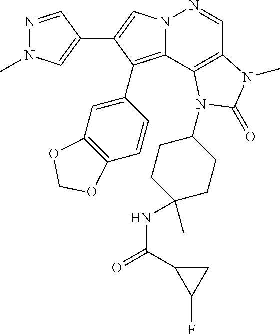 |
| I-64 | 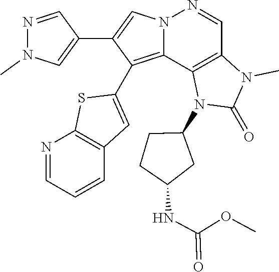 |
| I-64′ | 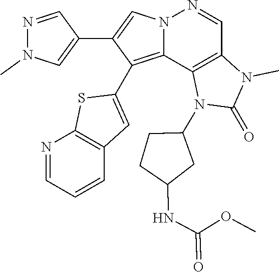 |
| I-65 | 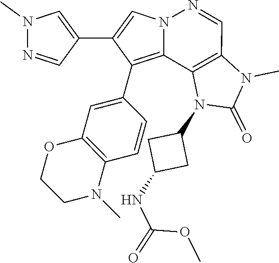 |
| I-65′ | 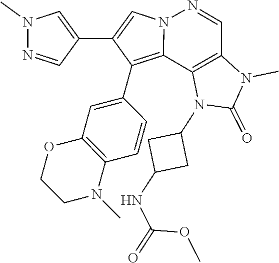 |
| I-66 | 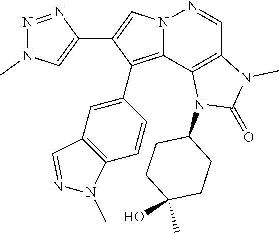 |
| I-66′ | 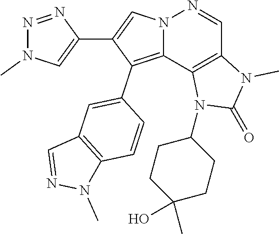 |
| I-67 | 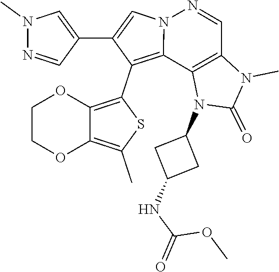 |
| I-67′ | 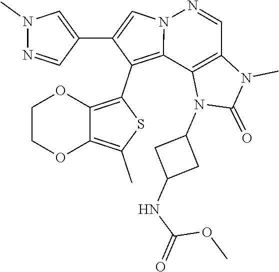 |
| I-68 | 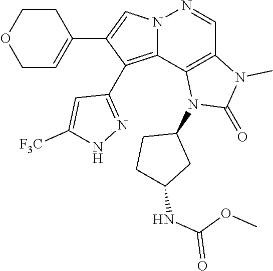 [SMILES: COC(=O)N[C@@H]1CC[C@@H](n2c(=O)n(C)c3cnn4cc(C5=CCOCC5)c(-c5cc(C(F)(F)F)[nH]n5)c4c32)C1] |
| I-68′ | 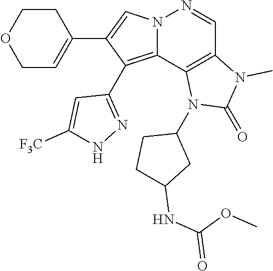 [SMILES: COC(=O)NC1CCC(n2c(=O)n(C)c3cnn4cc(C5=CCOCC5)c(-c5cc(C(F)(F)F)[nH]n5)c4c32)C1] |
| I-69 | 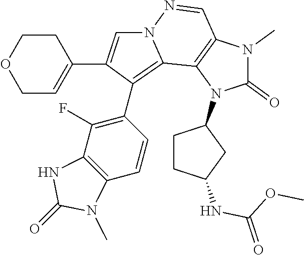 [SMILES: COC(=O)N[C@@H]1CC[C@@H](n2c(=O)n(C)c3cnn4cc(C5=CCOCC5)c(-c5ccc6c([nH]c(=O)n6C)c5F)c4c32)C1] |
| I-69′ | 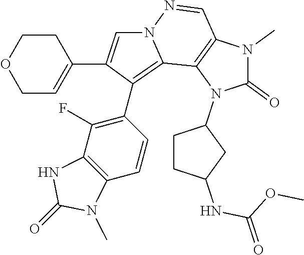 [SMILES: COC(=O)NC1CCC(n2c(=O)n(C)c3cnn4cc(C5=CCOCC5)c(-c5ccc6c([nH]c(=O)n6C)c5F)c4c32)C1] |
| I-70 | 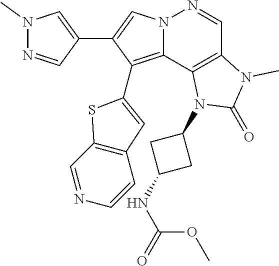 |
| I-70′ | 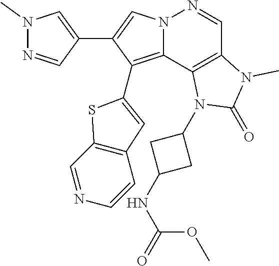 |
| I-71 | 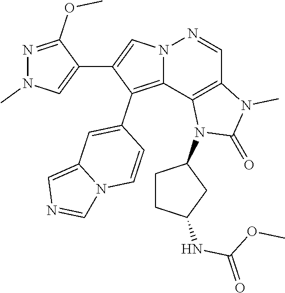 |
| I-71′ | 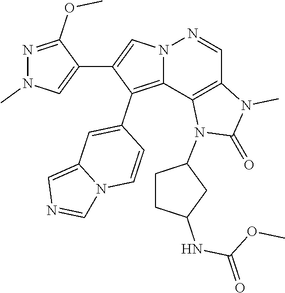 |
| I-72 | 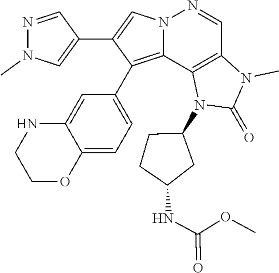 |
| I-72′ | 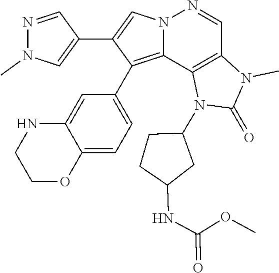 |
| I-73 | 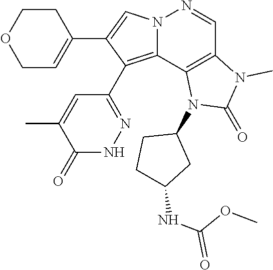 [SMILES: COC(=O)N[C@@H]1CC[C@@H](n2c(=O)n(C)c3cnn4cc(C5=CCOCC5)c(-c5cc(C)c(=O)[nH]n5)c4c32)C1] |
| I-73′ | 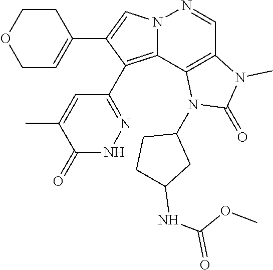 [SMILES: COC(=O)NC1CCC(n2c(=O)n(C)c3cnn4cc(C5=CCOCC5)c(-c5cc(C)c(=O)[nH]n5)c4c32)C1] |
| I-74 | 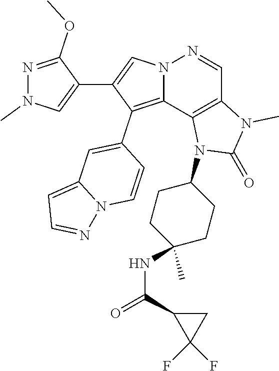 |
| I-74′ | 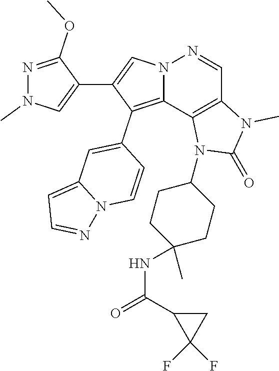 |
| I-75 | 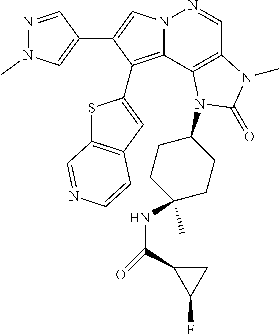 |
| I-75′ | 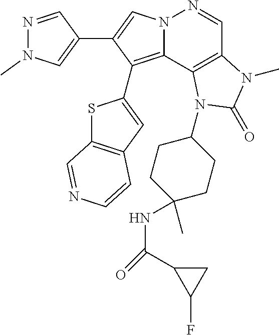 |
| I-76 | 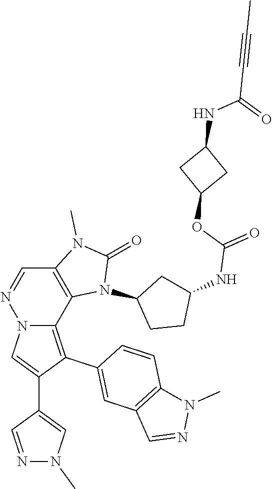 [SMILES: CC#CC(=O)N[C@H]1C[C@@H](OC(=O)N[C@@H]2CC[C@@H](n3c(=O)n(C)c4cnn5cc(-c6cnn(C)c6)c(-c6ccc7c(cnn7C)c6)c5c43)C2)C1] |
| I-76′ | 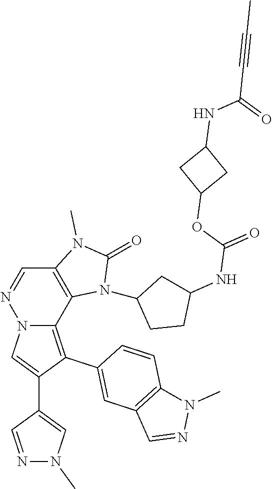 [SMILES: CC#CC(=O)NC1CC(OC(=O)NC2CCC(n3c(=O)n(C)c4cnn5cc(-c6cnn(C)c6)c(-c6ccc7c(cnn7C)c6)c5c43)C2)C1] |
| I-77 | 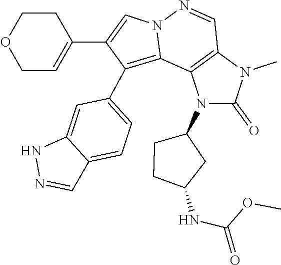 [SMILES: COC(=O)N[C@@H]1CC[C@@H](n2c(=O)n(C)c3cnn4cc(C5=CCOCC5)c(-c5ccc6cn[nH]c6c5)c4c32)C1] |
| I-77′ | 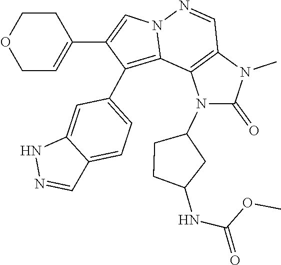 [SMILES: COC(=O)NC1CCC(n2c(=O)n(C)c3cnn4cc(C5=CCOCC5)c(-c5ccc6cn[nH]c6c5)c4c32)C1] |
| I-78 | 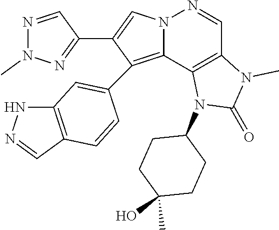 |
| I-78′ | 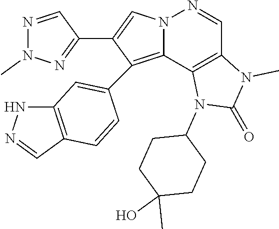 |
| I-79 | 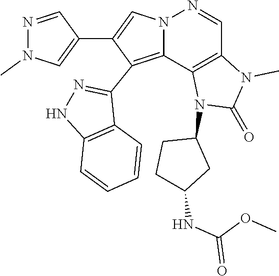 |
| I-79′ | 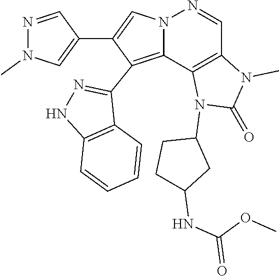 |
| I-80 | 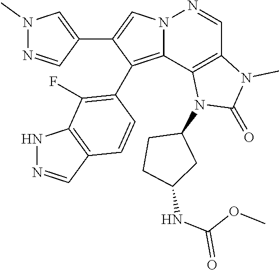 |
| I-80′ | 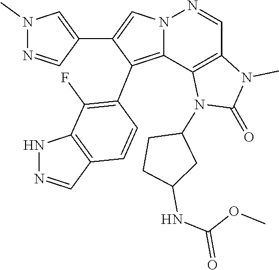 |
| I-81 | 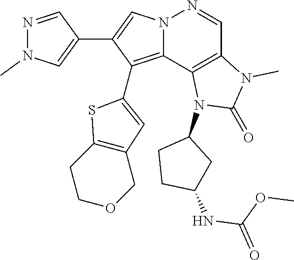 |
| I-81′ | 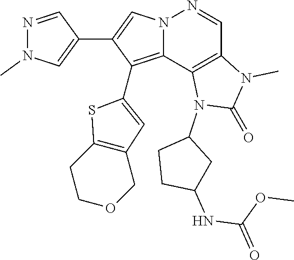 |
| I-82 | 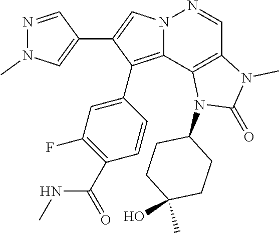 |
| I-82′ | 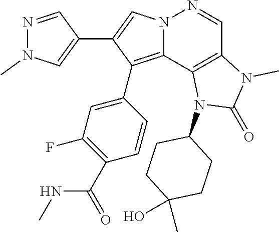 |
| I-83 | 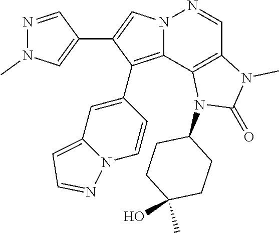 |
| I-83′ | 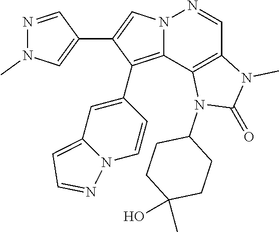 |
| I-84 | 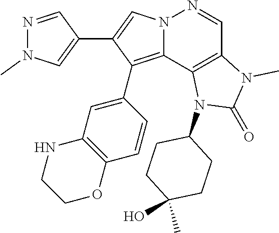 |
| I-84′ | 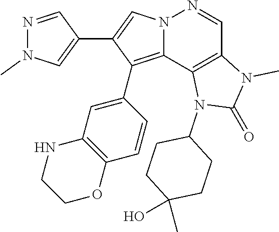 |
| I-85 | 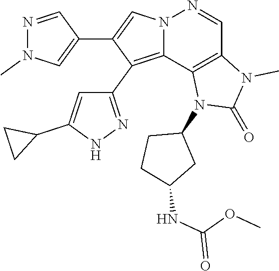 |
| I-85′ | 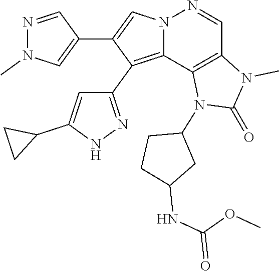 |
| I-86 | 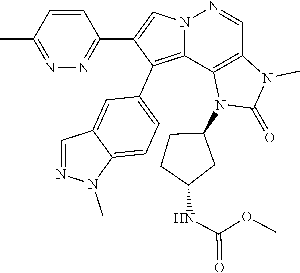 |
| I-86′ | 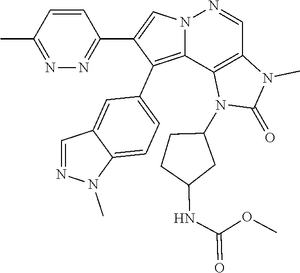 |
| I-87 | 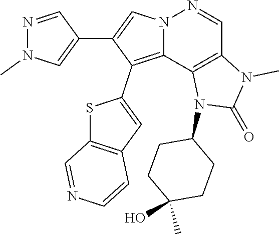 |
| I-87′ | 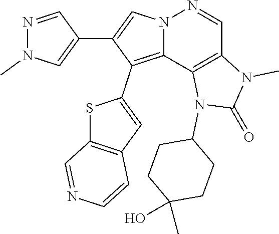 |
| I-88 | 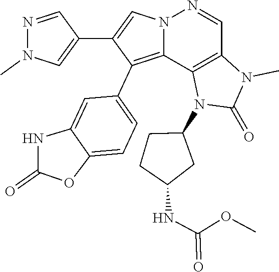 |
| I-88′ | 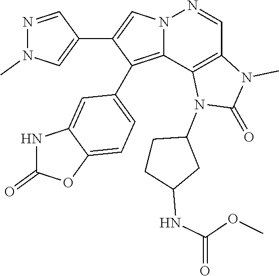 |
| I-89 | 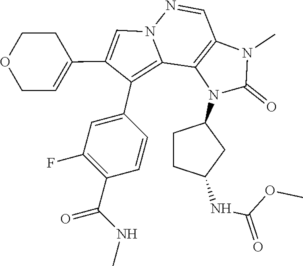 [SMILES: CNC(=O)c1ccc(-c2c(C3=CCOCC3)cn3ncc4c(c23)n([C@@H]2CC[C@@H](NC(=O)OC)C2)c(=O)n4C)cc1F] |
| I-89′ | 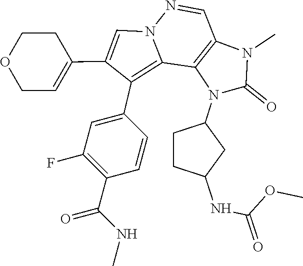 [SMILES: CNC(=O)c1ccc(-c2c(C3=CCOCC3)cn3ncc4c(c23)n(C2CCC(NC(=O)OC)C2)c(=O)n4C)cc1F] |
| I-90 | 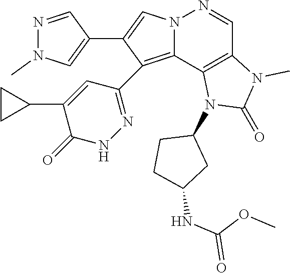 [SMILES: COC(=O)N[C@@H]1CC[C@@H](n2c(=O)n(C)c3cnn4cc(-c5cnn(C)c5)c(-c5cc(C6CC6)c(=O)[nH]n5)c4c32)C1] |
| I-90′ | 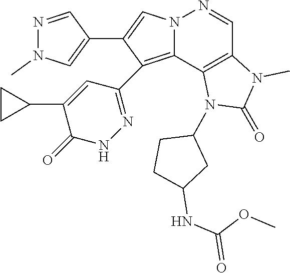 [SMILES: COC(=O)NC1CCC(n2c(=O)n(C)c3cnn4cc(-c5cnn(C)c5)c(-c5cc(C6CC6)c(=O)[nH]n5)c4c32)C1] |
| I-91 | 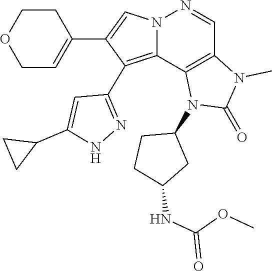 [SMILES: COC(=O)N[C@@H]1CC[C@@H](n2c(=O)n(C)c3cnn4cc(C5=CCOCC5)c(-c5cc(C6CC6)[nH]n5)c4c32)C1] |
| I-91′ | 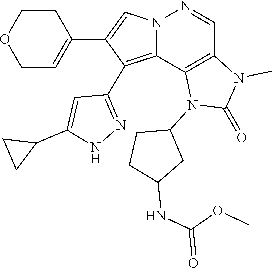 [SMILES: COC(=O)NC1CCC(n2c(=O)n(C)c3cnn4cc(C5=CCOCC5)c(-c5cc(C6CC6)[nH]n5)c4c32)C1] |
| I-93 | 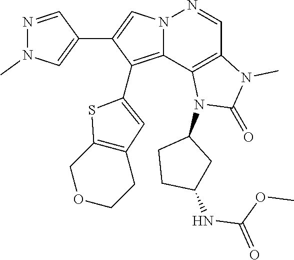 |
| I-93′ | 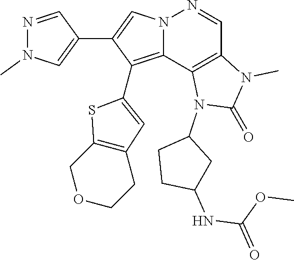 |
| I-94 | 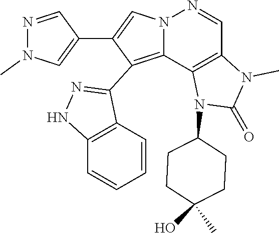 |
| I-94′ | 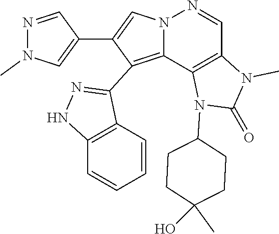 |
| I-95 | 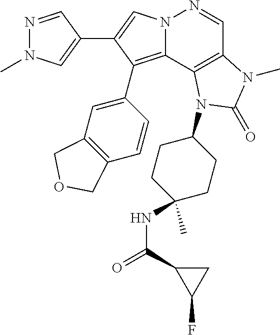 |
| I-95′ | 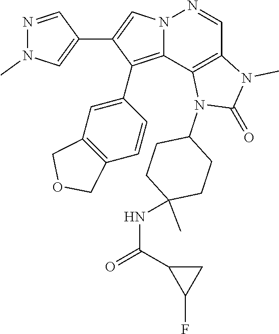 |
| I-96 | 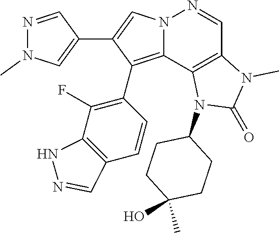 |
| I-96′ | 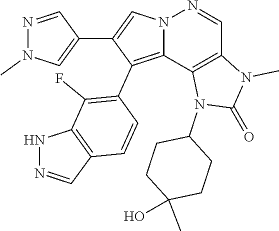 |
| I-97 | 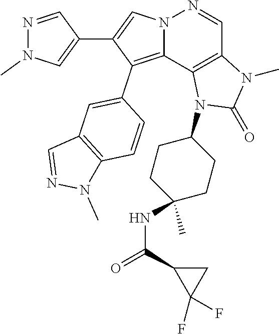 |
| I-97′ | 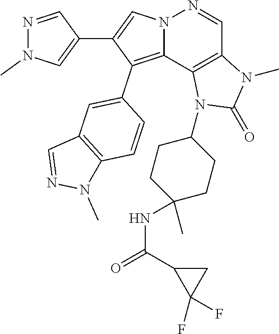 |
| I-98 | 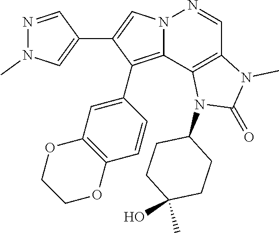 |
| I-98′ | 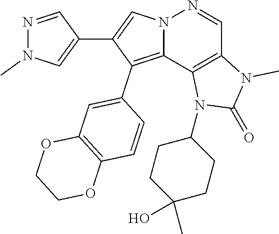 |
| I-99 | 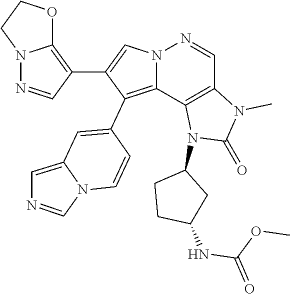 [SMILES: COC(=O)N[C@@H]1CC[C@@H](n2c(=O)n(C)c3cnn4cc(-c5cnn6c5OCC6)c(-c5ccn6cncc6c5)c4c32)C1] |
| I-99′ | 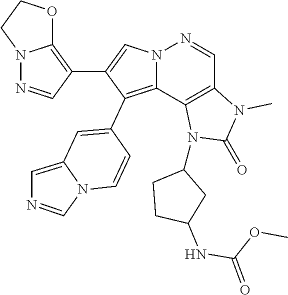 [SMILES: COC(=O)NC1CCC(n2c(=O)n(C)c3cnn4cc(-c5cnn6c5OCC6)c(-c5ccn6cncc6c5)c4c32)C1] |
| I-100 | 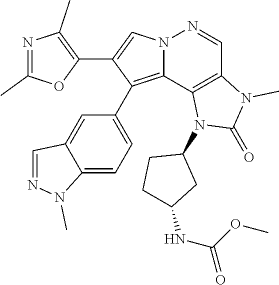 |
| I-100′ | 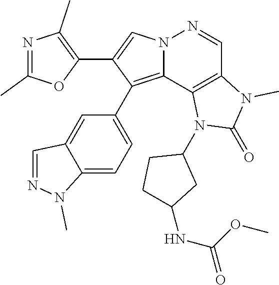 |
| I-101 | 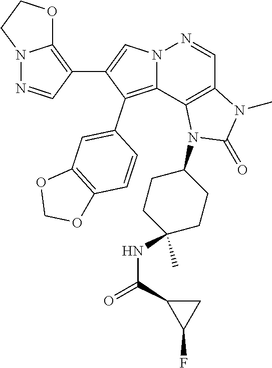 [SMILES: Cn1c(=O)n([C@H]2CC[C@@](C)(NC(=O)[C@H]3C[C@H]3F)CC2)c2c1cnn1cc(-c3cnn4c3OCC4)c(-c3ccc4c(c3)OCO4)c21] |
| I-101′ | 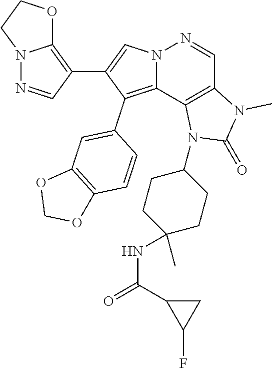 [SMILES: Cn1c(=O)n(C2CCC(C)(NC(=O)C3CC3F)CC2)c2c1cnn1cc(-c3cnn4c3OCC4)c(-c3ccc4c(c3)OCO4)c21] |
| I-102 | 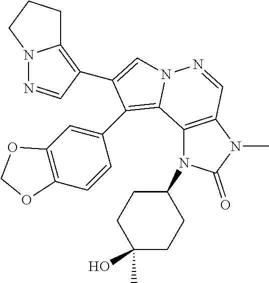 [SMILES: Cn1c(=O)n([C@H]2CC[C@@](C)(O)CC2)c2c1cnn1cc(-c3cnn4c3CCC4)c(-c3ccc4c(c3)OCO4)c21] |
| I-102′ | 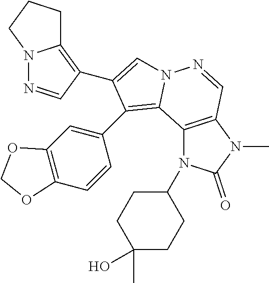 [SMILES: Cn1c(=O)n(C2CCC(C)(O)CC2)c2c1cnn1cc(-c3cnn4c3CCC4)c(-c3ccc4c(c3)OCO4)c21] |
| I-103 | 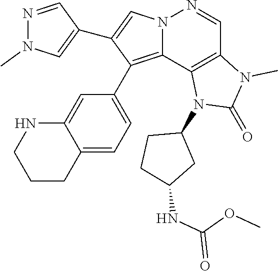 |
| I-103′ | 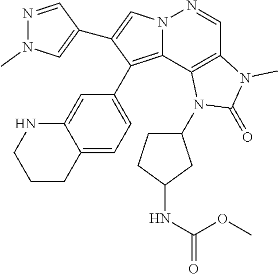 |
| I-104 | 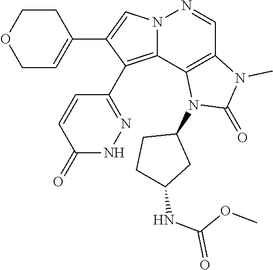 [SMILES: COC(=O)N[C@@H]1CC[C@@H](n2c(=O)n(C)c3cnn4cc(C5=CCOCC5)c(-c5ccc(=O)[nH]n5)c4c32)C1] |
| I-104′ | 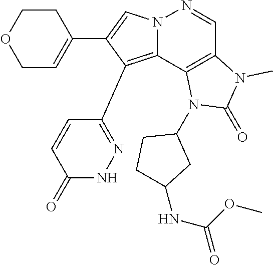 [SMILES: COC(=O)NC1CCC(n2c(=O)n(C)c3cnn4cc(C5=CCOCC5)c(-c5ccc(=O)[nH]n5)c4c32)C1] |
| I-105 | 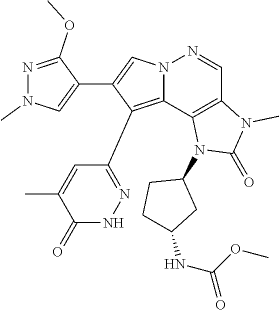 |
| I-105′ | 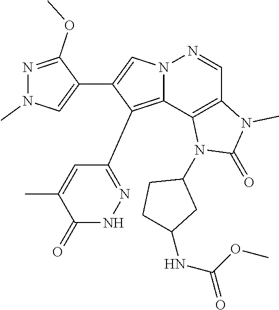 |
| I-106 | 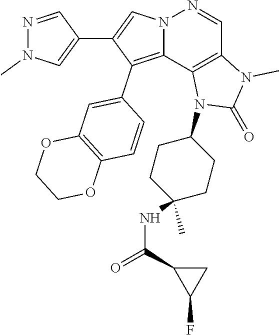 |
| I-106′ | 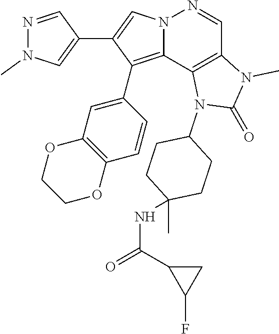 |
| I-107 | 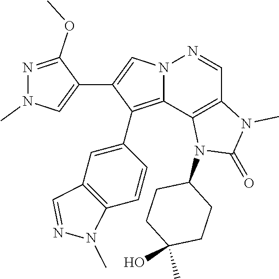 |
| I-107′ | 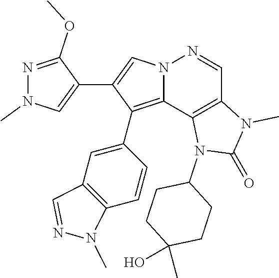 |
| I-108 | 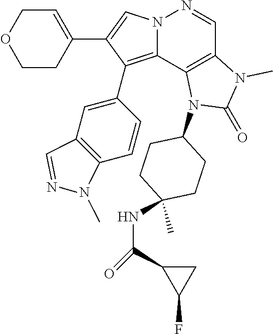 [SMILES: Cn1ncc2cc(-c3c(C4=CCOCC4)cn4ncc5c(c34)n([C@H]3CC[C@@](C)(NC(=O)[C@H]4C[C@H]4F)CC3)c(=O)n5C)ccc21] |
| I-108′ | 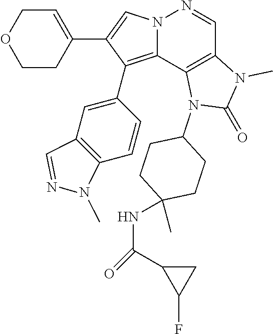 [SMILES: Cn1ncc2cc(-c3c(C4=CCOCC4)cn4ncc5c(c34)n(C3CCC(C)(NC(=O)C4CC4F)CC3)c(=O)n5C)ccc21] |
| I-109 | 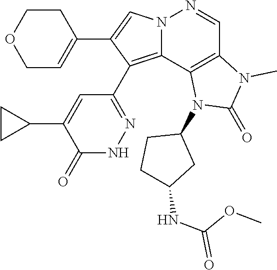 [SMILES: COC(=O)N[C@@H]1CC[C@@H](n2c(=O)n(C)c3cnn4cc(C5=CCOCC5)c(-c5cc(C6CC6)c(=O)[nH]n5)c4c32)C1] |
| I-109′ | 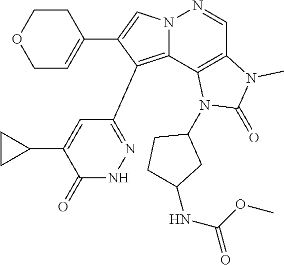 [SMILES: COC(=O)NC1CCC(n2c(=O)n(C)c3cnn4cc(C5=CCOCC5)c(-c5cc(C6CC6)c(=O)[nH]n5)c4c32)C1] |
| I-110 | 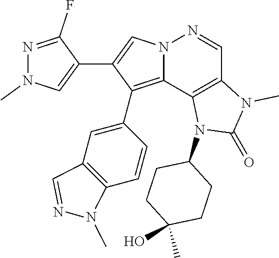 |
| I-110′ | 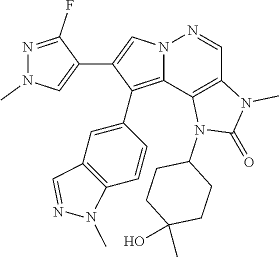 |
| I-111 | 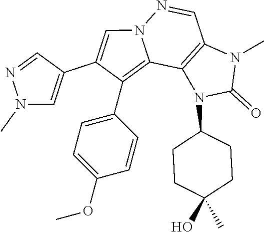 |
| I-111′ | 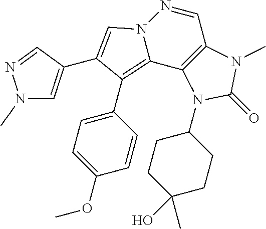 |
| I-112 | 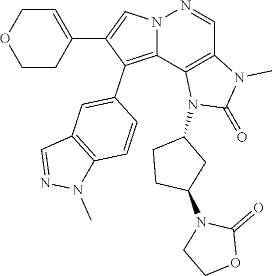 [SMILES: Cn1ncc2cc(-c3c(C4=CCOCC4)cn4ncc5c(c34)n([C@H]3CC[C@H](N4CCOC4=O)C3)c(=O)n5C)ccc21] |
| I-112′ | 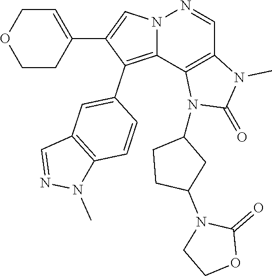 [SMILES: Cn1ncc2cc(-c3c(C4=CCOCC4)cn4ncc5c(c34)n(C3CCC(N4CCOC4=O)C3)c(=O)n5C)ccc21] |
| I-113 | 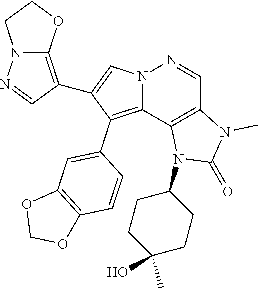 [SMILES: Cn1c(=O)n([C@H]2CC[C@@](C)(O)CC2)c2c1cnn1cc(-c3cnn4c3OCC4)c(-c3ccc4c(c3)OCO4)c21] |
| I-113′ | 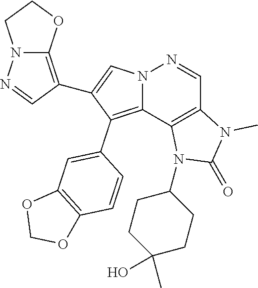 [SMILES: Cn1c(=O)n(C2CCC(C)(O)CC2)c2c1cnn1cc(-c3cnn4c3OCC4)c(-c3ccc4c(c3)OCO4)c21] |
| I-114 | 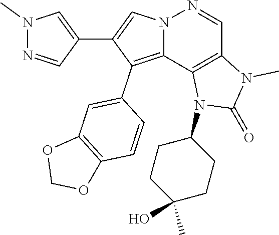 |
| I-114′ | 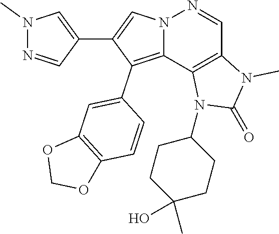 |
| I-115 | 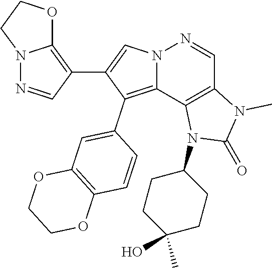 [SMILES: Cn1c(=O)n([C@H]2CC[C@@](C)(O)CC2)c2c1cnn1cc(-c3cnn4c3OCC4)c(-c3ccc4c(c3)OCCO4)c21] |
| I-115′ | 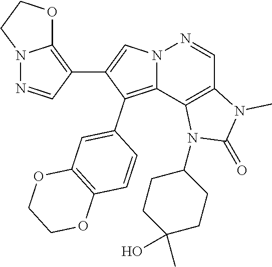 [SMILES: Cn1c(=O)n(C2CCC(C)(O)CC2)c2c1cnn1cc(-c3cnn4c3OCC4)c(-c3ccc4c(c3)OCCO4)c21] |
| I-116 | 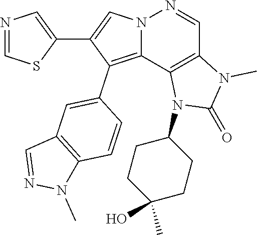 [SMILES: Cn1ncc2cc(-c3c(-c4cncs4)cn4ncc5c(c34)n([C@H]3CC[C@@](C)(O)CC3)c(=O)n5C)ccc21] |
| I-116′ | 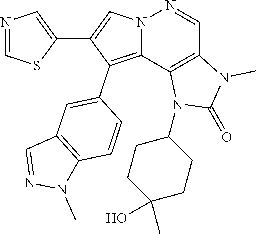 [SMILES: Cn1ncc2cc(-c3c(-c4cncs4)cn4ncc5c(c34)n(C3CCC(C)(O)CC3)c(=O)n5C)ccc21] |
| I-117 | 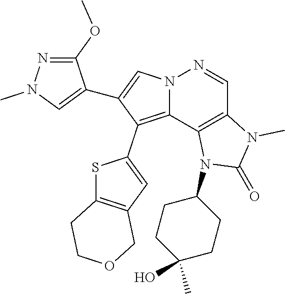 |
| I-117′ | 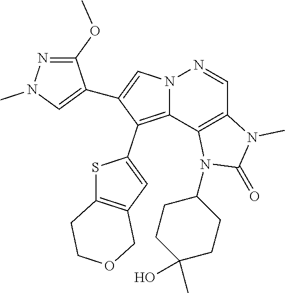 |
| I-118 | 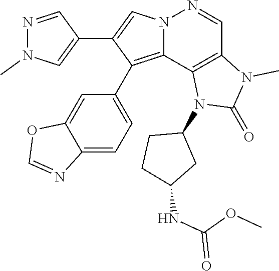 |
| I-118′ | 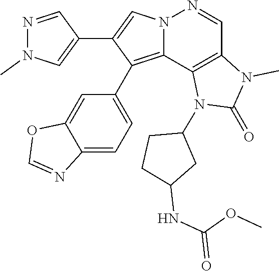 |
| I-119 | 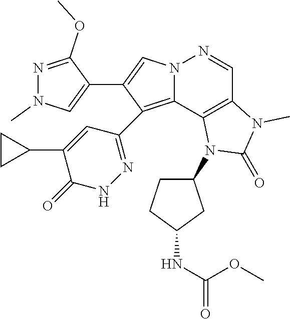 [SMILES: COC(=O)N[C@@H]1CC[C@@H](n2c(=O)n(C)c3cnn4cc(-c5cn(C)nc5OC)c(-c5cc(C6CC6)c(=O)[nH]n5)c4c32)C1] |
| I-119′ | 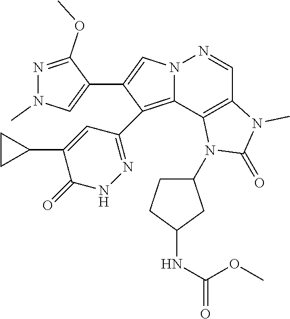 [SMILES: COC(=O)NC1CCC(n2c(=O)n(C)c3cnn4cc(-c5cn(C)nc5OC)c(-c5cc(C6CC6)c(=O)[nH]n5)c4c32)C1] |
| I-120 | 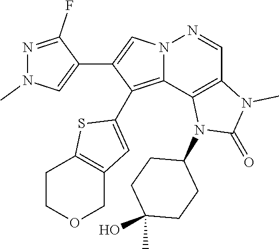 |
| I-120′ | 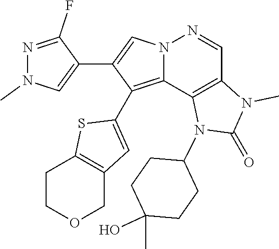 |
| I-121 | 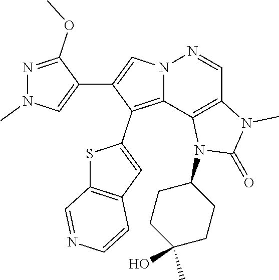 |
| I-121′ | 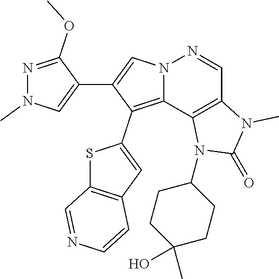 |
| I-122 | 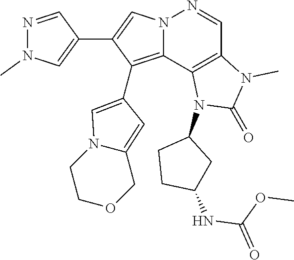 |
| I-122′ | 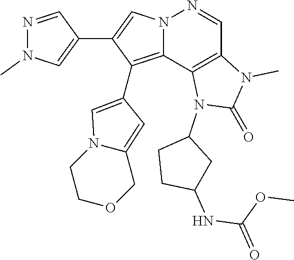 |
| I-123 | 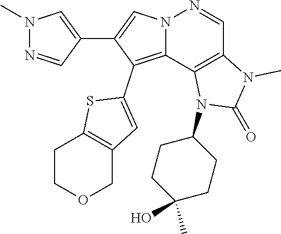 |
| I-123′ | 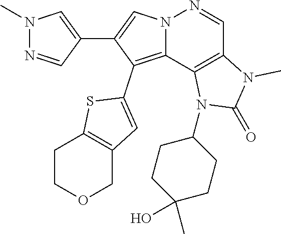 |
| I-124 | 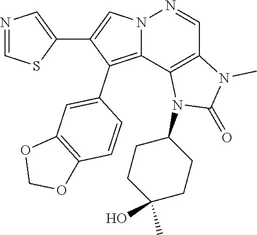 [SMILES: Cn1c(=O)n([C@H]2CC[C@@](C)(O)CC2)c2c1cnn1cc(-c3cncs3)c(-c3ccc4c(c3)OCO4)c21] |
| I-124′ | 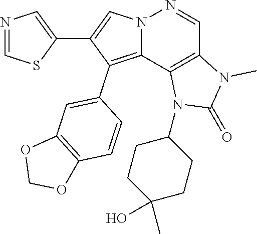 [SMILES: Cn1c(=O)n(C2CCC(C)(O)CC2)c2c1cnn1cc(-c3cncs3)c(-c3ccc4c(c3)OCO4)c21] |
| I-125 | 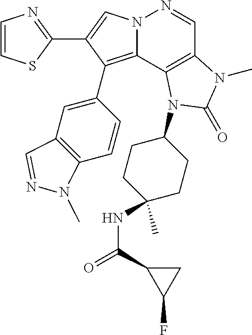 [SMILES: Cn1ncc2cc(-c3c(-c4nccs4)cn4ncc5c(c34)n([C@H]3CC[C@@](C)(NC(=O)[C@H]4C[C@H]4F)CC3)c(=O)n5C)ccc21] |
| I-125′ | 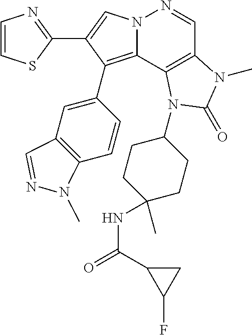 [SMILES: Cn1ncc2cc(-c3c(-c4nccs4)cn4ncc5c(c34)n(C3CCC(C)(NC(=O)C4CC4F)CC3)c(=O)n5C)ccc21] |
| I-126 | 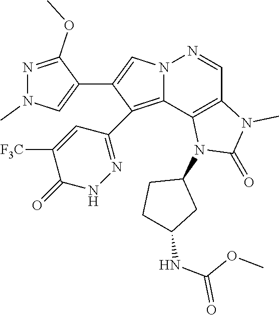 |
| I-126′ | 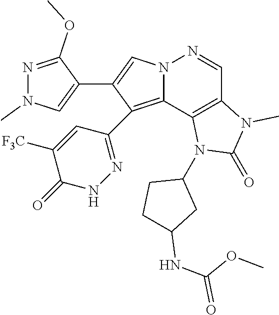 |
| I-127 | 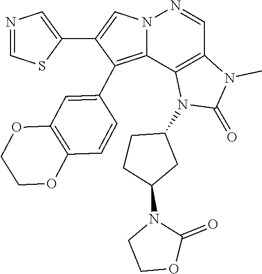 [SMILES: Cn1c(=O)n([C@H]2CC[C@H](N3CCOC3=O)C2)c2c1cnn1cc(-c3cncs3)c(-c3ccc4c(c3)OCCO4)c21] |
| I-127′ | 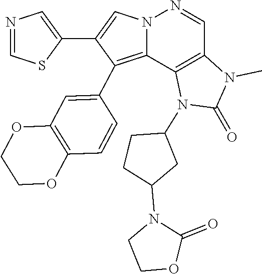 [SMILES: Cn1c(=O)n(C2CCC(N3CCOC3=O)C2)c2c1cnn1cc(-c3cncs3)c(-c3ccc4c(c3)OCCO4)c21] |
| I-128 | 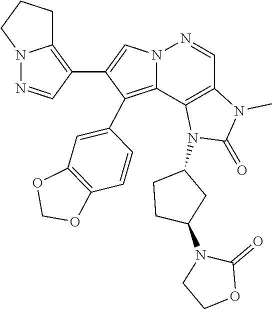 [SMILES: Cn1c(=O)n([C@H]2CC[C@H](N3CCOC3=O)C2)c2c1cnn1cc(-c3cnn4c3CCC4)c(-c3ccc4c(c3)OCO4)c21] |
| I-128′ | 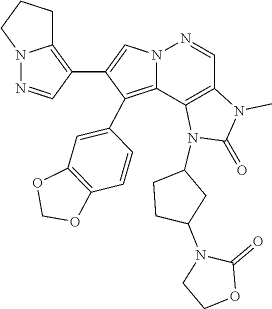 [SMILES: Cn1c(=O)n(C2CCC(N3CCOC3=O)C2)c2c1cnn1cc(-c3cnn4c3CCC4)c(-c3ccc4c(c3)OCO4)c21] |
| I-129 | 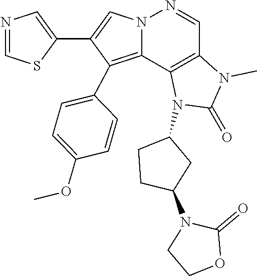 [SMILES: COc1ccc(-c2c(-c3cncs3)cn3ncc4c(c23)n([C@H]2CC[C@H](N3CCOC3=O)C2)c(=O)n4C)cc1] |
| I-129′ | 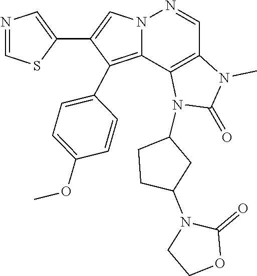 [SMILES: COc1ccc(-c2c(-c3cncs3)cn3ncc4c(c23)n(C2CCC(N3CCOC3=O)C2)c(=O)n4C)cc1] |
| I-130 | 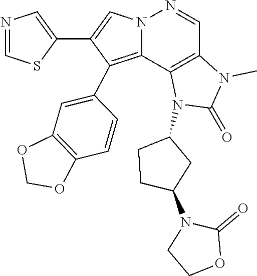 [SMILES: Cn1c(=O)n([C@H]2CC[C@H](N3CCOC3=O)C2)c2c1cnn1cc(-c3cncs3)c(-c3ccc4c(c3)OCO4)c21] |
| I-130′ | 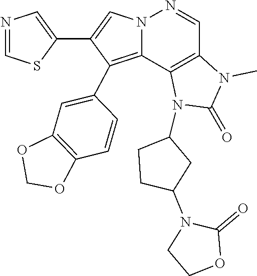 [SMILES: Cn1c(=O)n(C2CCC(N3CCOC3=O)C2)c2c1cnn1cc(-c3cncs3)c(-c3ccc4c(c3)OCO4)c21] |
| I-131 | 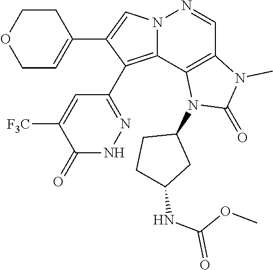 [SMILES: COC(=O)N[C@@H]1CC[C@@H](n2c(=O)n(C)c3cnn4cc(C5=CCOCC5)c(-c5cc(C(F)(F)F)c(=O)[nH]n5)c4c32)C1] |
| I-131′ | 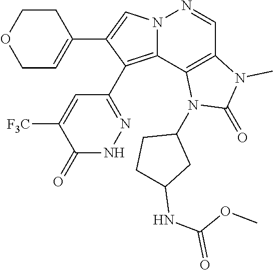 [SMILES: COC(=O)NC1CCC(n2c(=O)n(C)c3cnn4cc(C5=CCOCC5)c(-c5cc(C(F)(F)F)c(=O)[nH]n5)c4c32)C1] |
| I-132 | 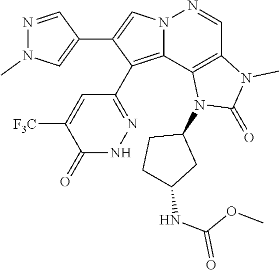 |
| I-132′ | 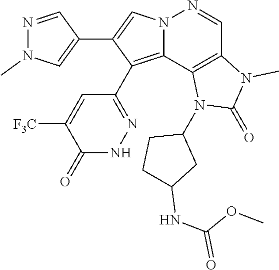 |
| I-133 | 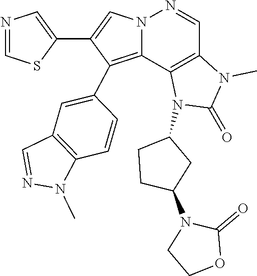 [SMILES: Cn1ncc2cc(-c3c(-c4cncs4)cn4ncc5c(c34)n([C@H]3CC[C@H](N4CCOC4=O)C3)c(=O)n5C)ccc21] |
| I-133′ | 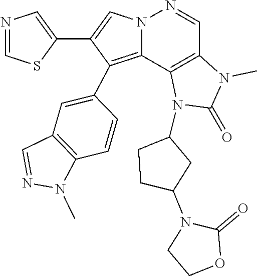 [SMILES: Cn1ncc2cc(-c3c(-c4cncs4)cn4ncc5c(c34)n(C3CCC(N4CCOC4=O)C3)c(=O)n5C)ccc21] |
| I-134 | 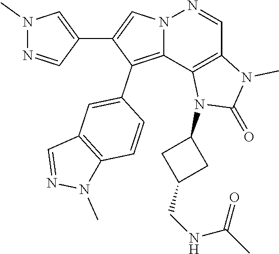 |
| I-134′ | 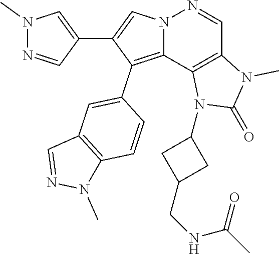 |
| I-135 | 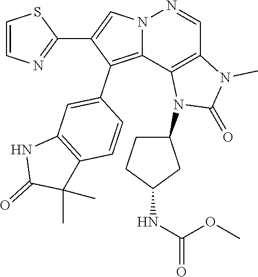 [SMILES: COC(=O)N[C@@H]1CC[C@@H](n2c(=O)n(C)c3cnn4cc(-c5nccs5)c(-c5ccc6c(c5)NC(=O)C6(C)C)c4c32)C1] |
| I-135′ | 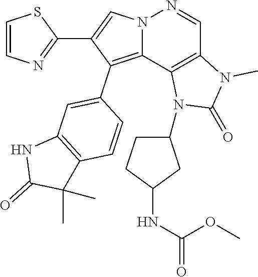 [SMILES: COC(=O)NC1CCC(n2c(=O)n(C)c3cnn4cc(-c5nccs5)c(-c5ccc6c(c5)NC(=O)C6(C)C)c4c32)C1] |
| I-136 | 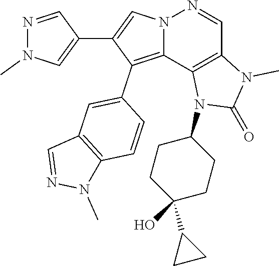 |
| I-136′ | 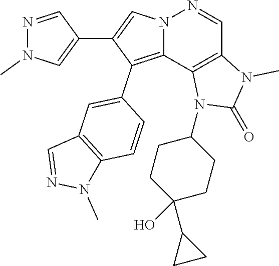 |
| I-137 | 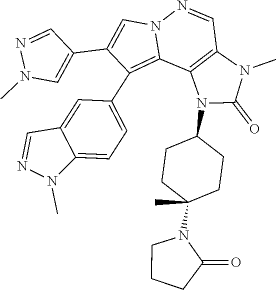 |
| I-137′ | 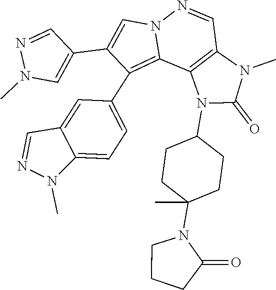 |
| I-138 | 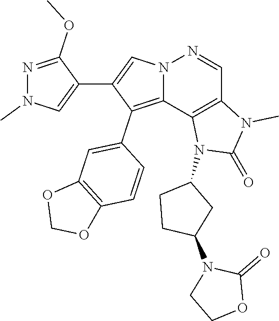 |
| I-138′ | 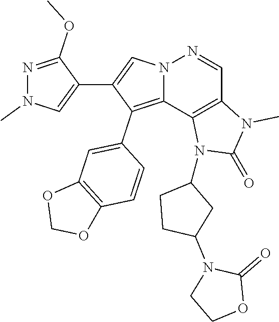 |
| I-139 | 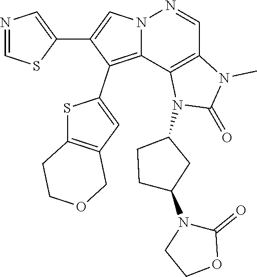 [SMILES: Cn1c(=O)n([C@H]2CC[C@H](N3CCOC3=O)C2)c2c1cnn1cc(-c3cncs3)c(-c3cc4c(s3)CCOC4)c21] |
| I-139′ | 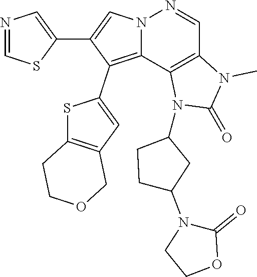 [SMILES: Cn1c(=O)n(C2CCC(N3CCOC3=O)C2)c2c1cnn1cc(-c3cncs3)c(-c3cc4c(s3)CCOC4)c21] |
| I-140 | 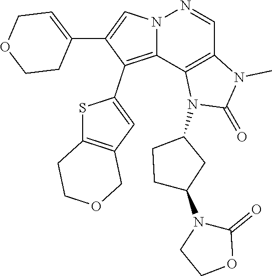 [SMILES: Cn1c(=O)n([C@H]2CC[C@H](N3CCOC3=O)C2)c2c1cnn1cc(C3=CCOCC3)c(-c3cc4c(s3)CCOC4)c21] |
| I-140′ | 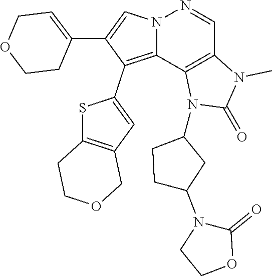 [SMILES: Cn1c(=O)n(C2CCC(N3CCOC3=O)C2)c2c1cnn1cc(C3=CCOCC3)c(-c3cc4c(s3)CCOC4)c21] |
| I-141 | 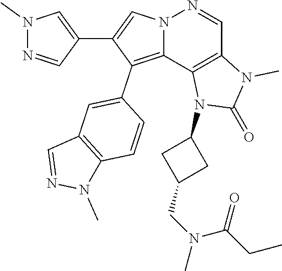 [SMILES: CCC(=O)N(C)C[C@H]1C[C@H](n2c(=O)n(C)c3cnn4cc(-c5cnn(C)c5)c(-c5ccc6c(cnn6C)c5)c4c32)C1] |
| I-141′ | 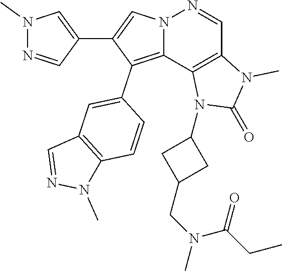 [SMILES: CCC(=O)N(C)CC1CC(n2c(=O)n(C)c3cnn4cc(-c5cnn(C)c5)c(-c5ccc6c(cnn6C)c5)c4c32)C1] |
| I-142 | 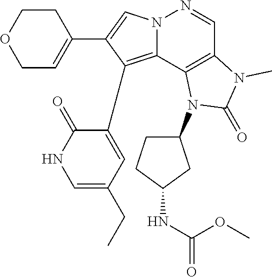 [SMILES: CCc1c[nH]c(=O)c(-c2c(C3=CCOCC3)cn3ncc4c(c23)n([C@@H]2CC[C@@H](NC(=O)OC)C2)c(=O)n4C)c1] |
| I-142′ | 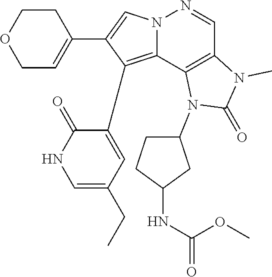 [SMILES: CCc1c[nH]c(=O)c(-c2c(C3=CCOCC3)cn3ncc4c(c23)n(C2CCC(NC(=O)OC)C2)c(=O)n4C)c1] |
| I-143 | 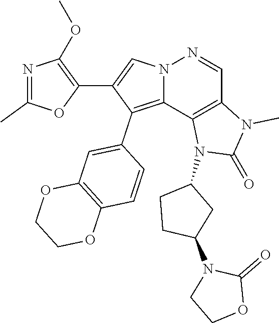 |
| I-143′ | 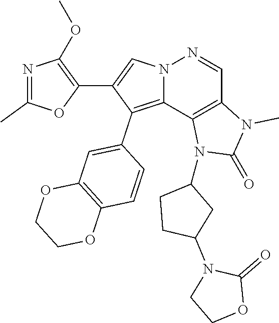 |
| I-144 | 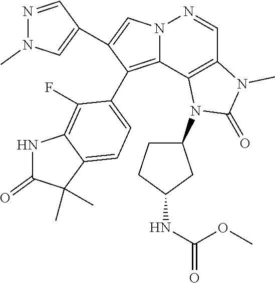 |
| I-144′ | 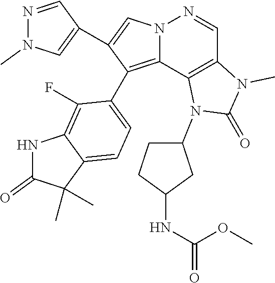 |
| I-145 | 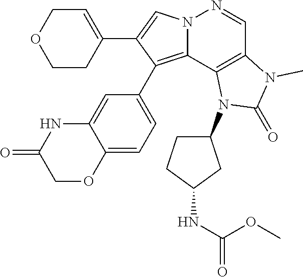 [SMILES: COC(=O)N[C@@H]1CC[C@@H](n2c(=O)n(C)c3cnn4cc(C5=CCOCC5)c(-c5ccc6c(c5)NC(=O)CO6)c4c32)C1] |
| I-145′ | 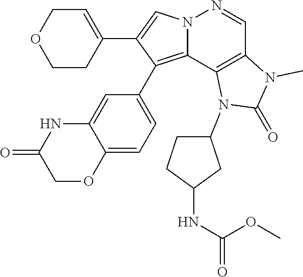 [SMILES: COC(=O)NC1CCC(n2c(=O)n(C)c3cnn4cc(C5=CCOCC5)c(-c5ccc6c(c5)NC(=O)CO6)c4c32)C1] |
| I-146 | 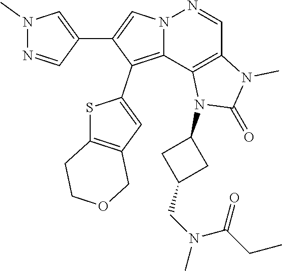 [SMILES: CCC(=O)N(C)C[C@H]1C[C@H](n2c(=O)n(C)c3cnn4cc(-c5cnn(C)c5)c(-c5cc6c(s5)CCOC6)c4c32)C1] |
| I-146′ | 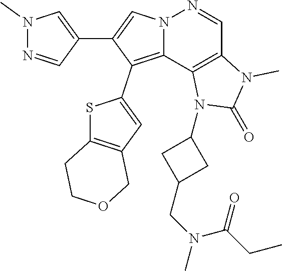 [SMILES: CCC(=O)N(C)CC1CC(n2c(=O)n(C)c3cnn4cc(-c5cnn(C)c5)c(-c5cc6c(s5)CCOC6)c4c32)C1] |
| I-147 | 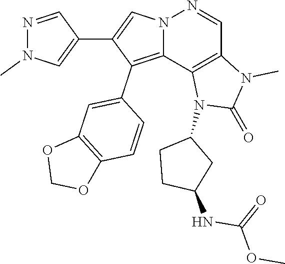 |
| I-147′ | 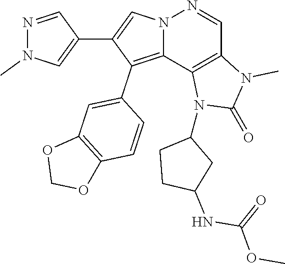 |
| I-148 | 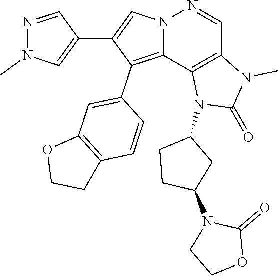 |
| I-148′ | 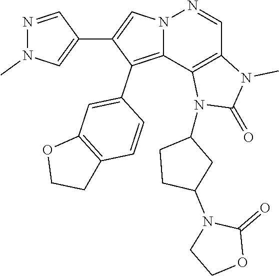 |
| I-149 | 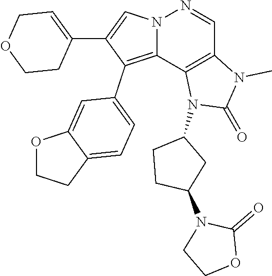 [SMILES: Cn1c(=O)n([C@H]2CC[C@H](N3CCOC3=O)C2)c2c1cnn1cc(C3=CCOCC3)c(-c3ccc4c(c3)OCC4)c21] |
| I-149′ | 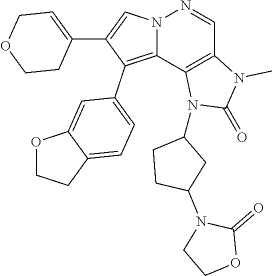 [SMILES: Cn1c(=O)n(C2CCC(N3CCOC3=O)C2)c2c1cnn1cc(C3=CCOCC3)c(-c3ccc4c(c3)OCC4)c21] |
| I-150 | 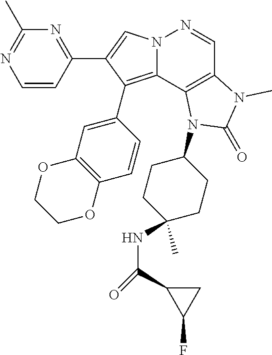 [SMILES: Cc1nccc(-c2cn3ncc4c(c3c2-c2ccc3c(c2)OCCO3)n([C@H]2CC[C@@](C)(NC(=O)[C@H]3C[C@H]3F)CC2)c(=O)n4C)n1] |
| I-150′ | 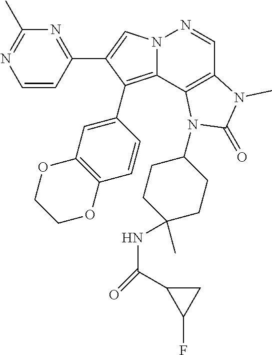 [SMILES: Cc1nccc(-c2cn3ncc4c(c3c2-c2ccc3c(c2)OCCO3)n(C2CCC(C)(NC(=O)C3CC3F)CC2)c(=O)n4C)n1] |
| I-151 | 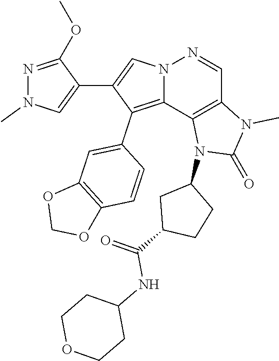 |
| I-151′ | 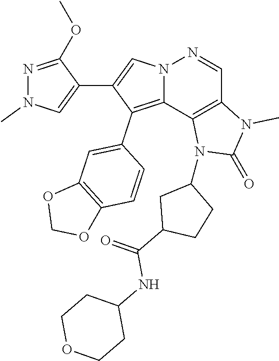 |
| I-152 | 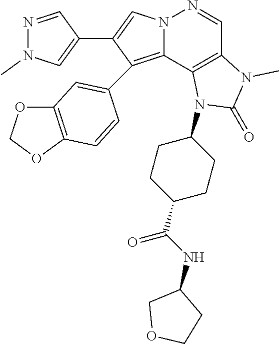 |
| I-152′ | 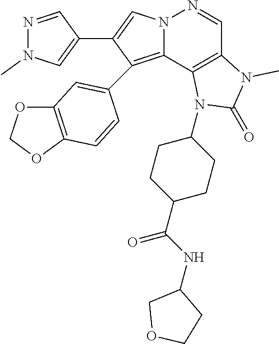 |
| I-153 | 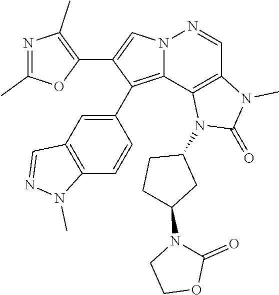 |
| I-153′ | 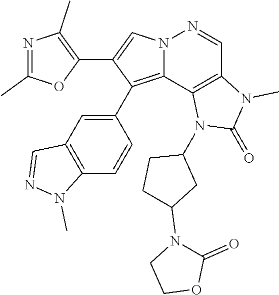 |
| I-154 | 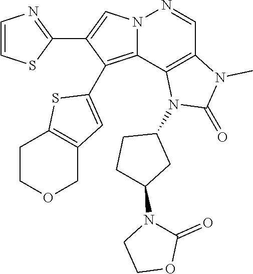 [SMILES: Cn1c(=O)n([C@H]2CC[C@H](N3CCOC3=O)C2)c2c1cnn1cc(-c3nccs3)c(-c3cc4c(s3)CCOC4)c21] |
| I-154′ | 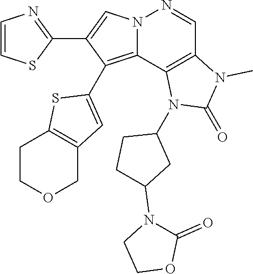 [SMILES: Cn1c(=O)n(C2CCC(N3CCOC3=O)C2)c2c1cnn1cc(-c3nccs3)c(-c3cc4c(s3)CCOC4)c21] |
| I-155 | 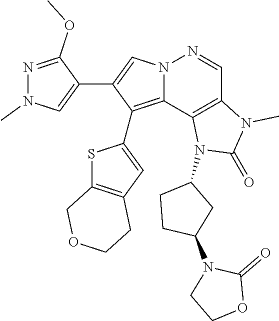 |
| I-155′ | 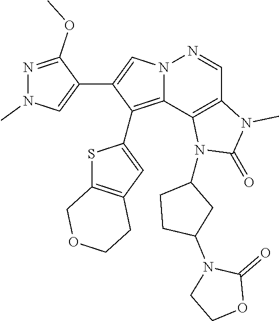 |
| I-156 | 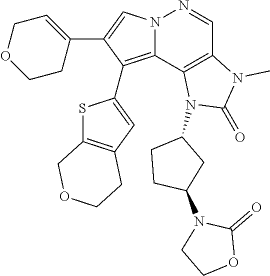 [SMILES: Cn1c(=O)n([C@H]2CC[C@H](N3CCOC3=O)C2)c2c1cnn1cc(C3=CCOCC3)c(-c3cc4c(s3)COCC4)c21] |
| I-156′ | 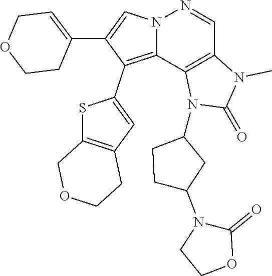 [SMILES: Cn1c(=O)n(C2CCC(N3CCOC3=O)C2)c2c1cnn1cc(C3=CCOCC3)c(-c3cc4c(s3)COCC4)c21] |
| I-157 | 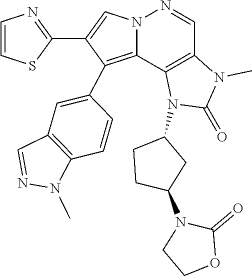 [SMILES: Cn1ncc2cc(-c3c(-c4nccs4)cn4ncc5c(c34)n([C@H]3CC[C@H](N4CCOC4=O)C3)c(=O)n5C)ccc21] |
| I-157′ | 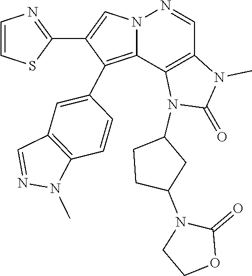 [SMILES: Cn1ncc2cc(-c3c(-c4nccs4)cn4ncc5c(c34)n(C3CCC(N4CCOC4=O)C3)c(=O)n5C)ccc21] |
| I-158 | 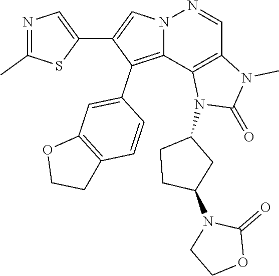 |
| I-158′ | 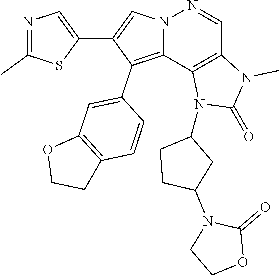 |
| I-159 | 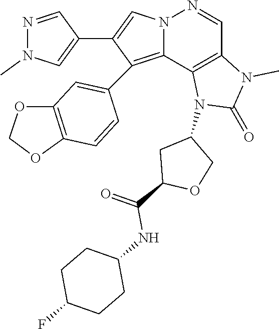 |
| I-159′ | 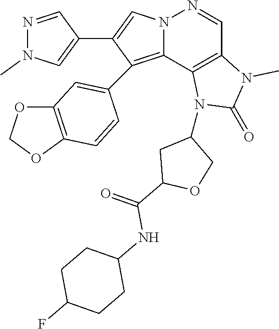 |
| I-160 | 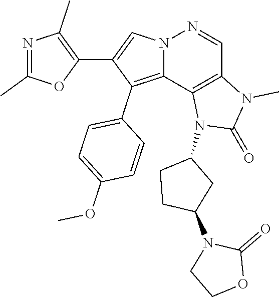 |
| I-160′ | 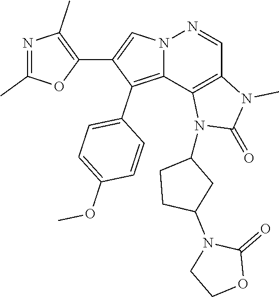 |
| I-161 | 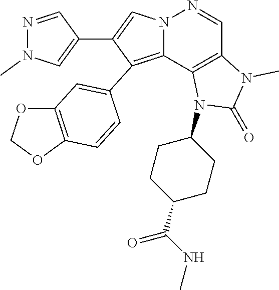 |
| I-161′ | 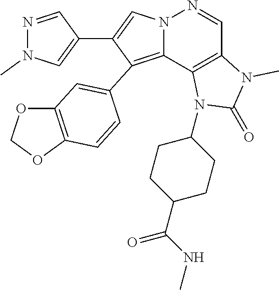 |
| I-162 | 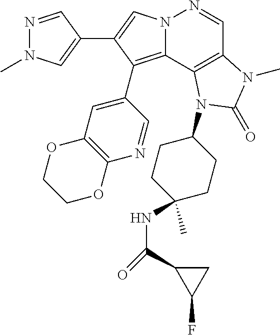 |
| I-162′ | 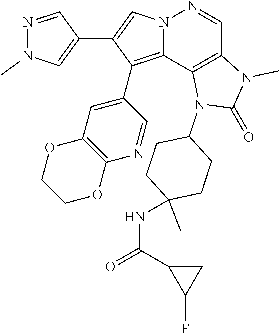 |
| I-163 | 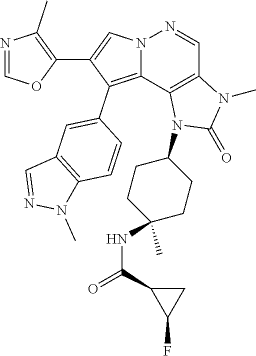 [SMILES: Cc1ncoc1-c1cn2ncc3c(c2c1-c1ccc2c(cnn2C)c1)n([C@H]1CC[C@@](C)(NC(=O)[C@H]2C[C@H]2F)CC1)c(=O)n3C] |
| I-163′ | 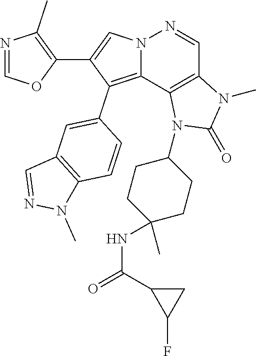 [SMILES: Cc1ncoc1-c1cn2ncc3c(c2c1-c1ccc2c(cnn2C)c1)n(C1CCC(C)(NC(=O)C2CC2F)CC1)c(=O)n3C] |
| I-164 | 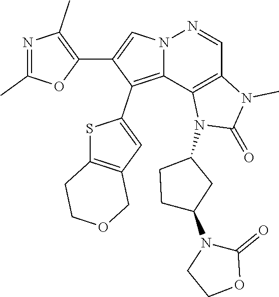 |
| I-164′ | 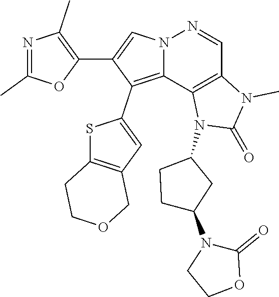 |
| I-165 | 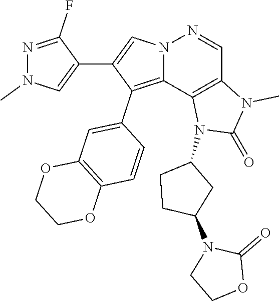 |
| I-165′ | 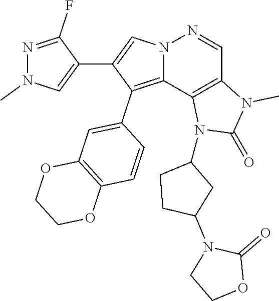 |
| I-166-1 | 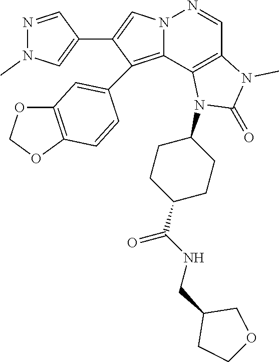 |
| I-166-2 | 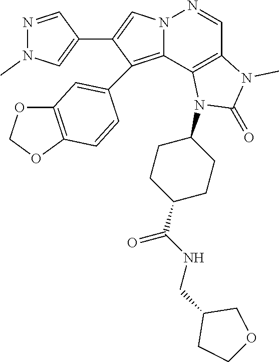 |
| I-166′ | 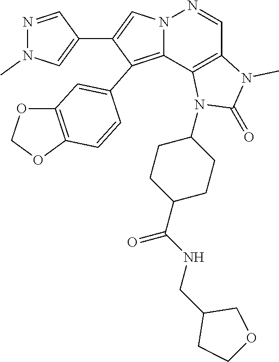 |
| I-167 | 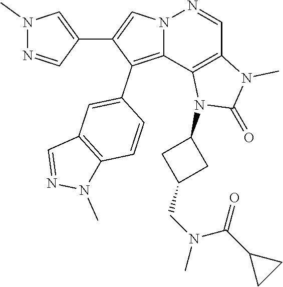 [SMILES: CN(C[C@H]1C[C@H](n2c(=O)n(C)c3cnn4cc(-c5cnn(C)c5)c(-c5ccc6c(cnn6C)c5)c4c32)C1)C(=O)C1CC1] |
| I-167′ | 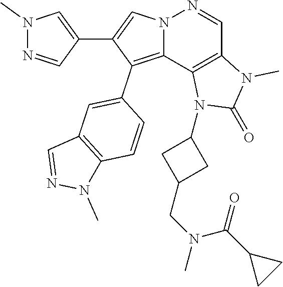 [SMILES: CN(CC1CC(n2c(=O)n(C)c3cnn4cc(-c5cnn(C)c5)c(-c5ccc6c(cnn6C)c5)c4c32)C1)C(=O)C1CC1] |
| I-168 | 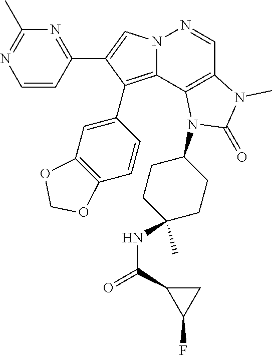 [SMILES: Cc1nccc(-c2cn3ncc4c(c3c2-c2ccc3c(c2)OCO3)n([C@H]2CC[C@@](C)(NC(=O)[C@H]3C[C@H]3F)CC2)c(=O)n4C)n1] |
| I-168′ | 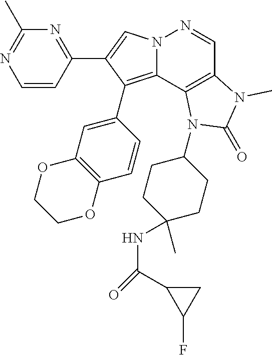 [SMILES: Cc1nccc(-c2cn3ncc4c(c3c2-c2ccc3c(c2)OCCO3)n(C2CCC(C)(NC(=O)C3CC3F)CC2)c(=O)n4C)n1] |
| I-169 | 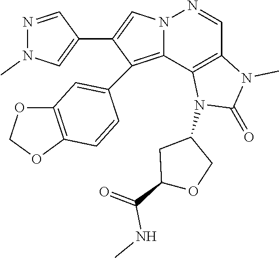 |
| I-169′ | 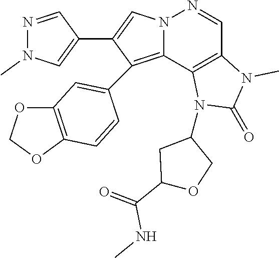 |
| I-170 | 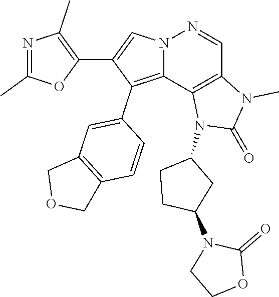 |
| I-170′ | 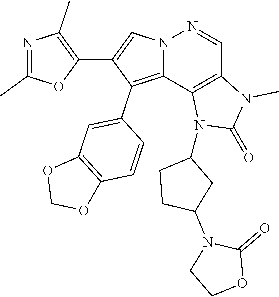 |
| I-171 | 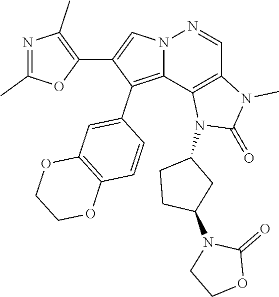 |
| I-171′ | 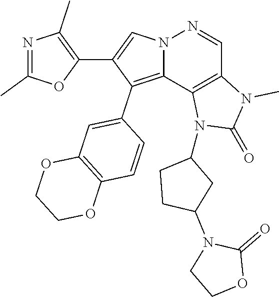 |
| I-172 | 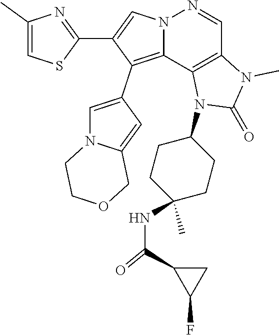 |
| I-172′ | 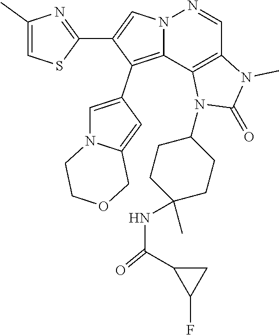 |
| I-173 | 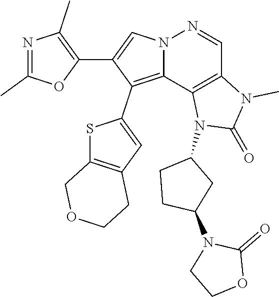 |
| I-173′ | 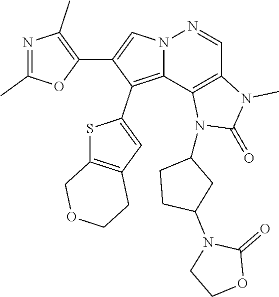 |
| I-174 | 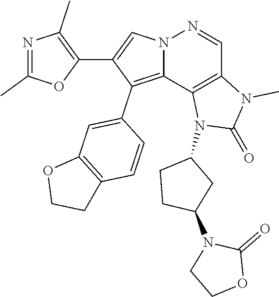 |
| I-174′ | 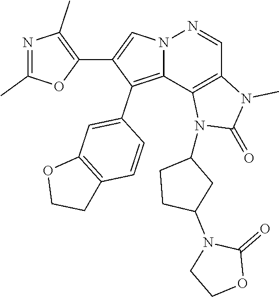 |
| I-175 | 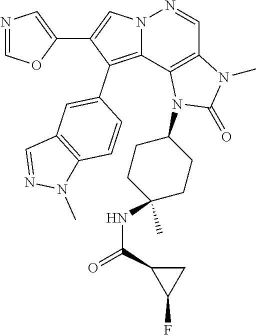 [SMILES: Cn1ncc2cc(-c3c(-c4cnco4)cn4ncc5c(c34)n([C@H]3CC[C@@](C)(NC(=O)[C@H]4C[C@H]4F)CC3)c(=O)n5C)ccc21] |
| I-175′ | 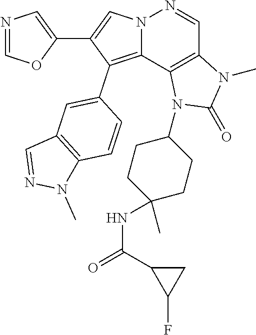 [SMILES: Cn1ncc2cc(-c3c(-c4cnco4)cn4ncc5c(c34)n(C3CCC(C)(NC(=O)C4CC4F)CC3)c(=O)n5C)ccc21] |
| I-176 | 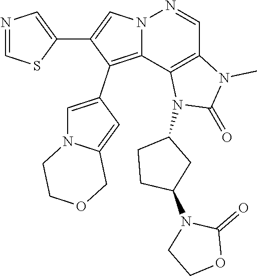 [SMILES: Cn1c(=O)n([C@H]2CC[C@H](N3CCOC3=O)C2)c2c1cnn1cc(-c3cncs3)c(-c3cc4n(c3)CCOC4)c21] |
| I-176′ | 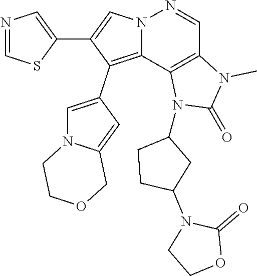 [SMILES: Cn1c(=O)n(C2CCC(N3CCOC3=O)C2)c2c1cnn1cc(-c3cncs3)c(-c3cc4n(c3)CCOC4)c21] |
| I-177 | 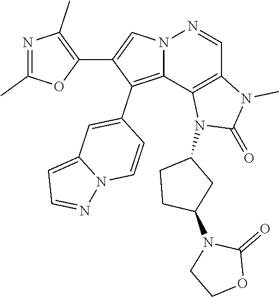 |
| I-177′ | 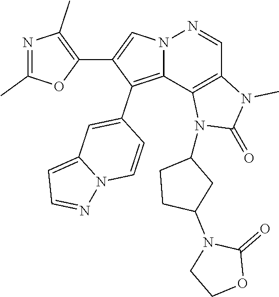 |
| I-178 | 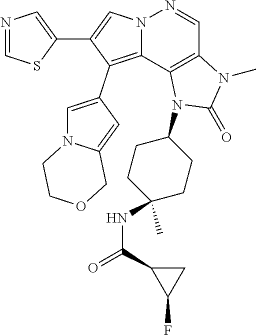 [SMILES: Cn1c(=O)n([C@H]2CC[C@@](C)(NC(=O)[C@H]3C[C@H]3F)CC2)c2c1cnn1cc(-c3cncs3)c(-c3cc4n(c3)CCOC4)c21] |
| I-178′ | 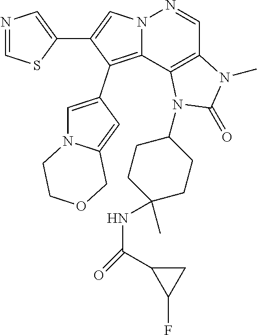 [SMILES: Cn1c(=O)n(C2CCC(C)(NC(=O)C3CC3F)CC2)c2c1cnn1cc(-c3cncs3)c(-c3cc4n(c3)CCOC4)c21] |
| I-179-1 | 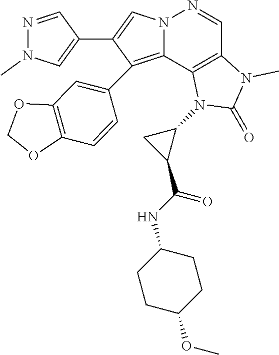 |
| I-179-2 | 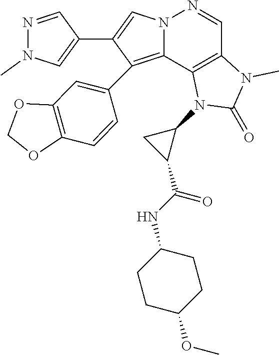 |
| I-179′ | 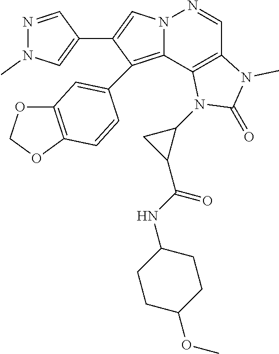 |
| I-180 | 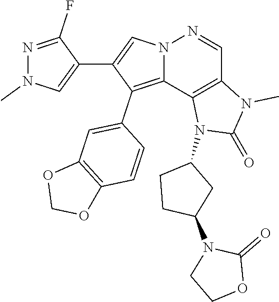 |
| I-180′ | 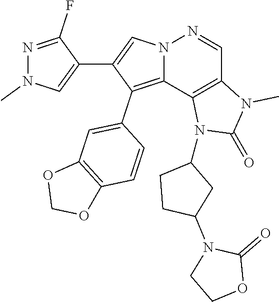 |
| I-181 | 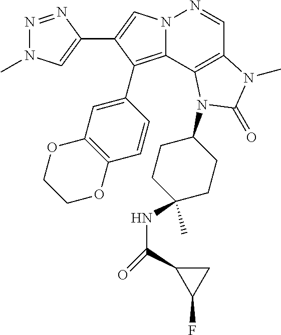 |
| I-181′ | 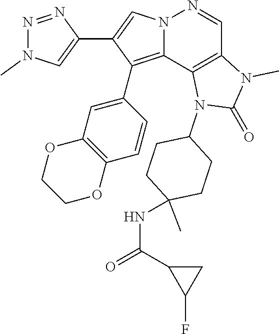 |
| I-182 | 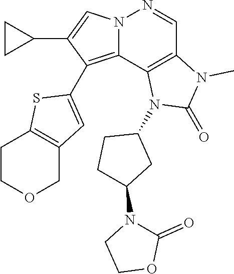 [SMILES: Cn1c(=O)n([C@H]2CC[C@H](N3CCOC3=O)C2)c2c1cnn1cc(C3CC3)c(-c3cc4c(s3)CCOC4)c21] |
| I-182′ | 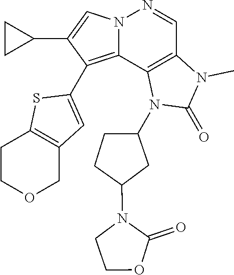 [SMILES: Cn1c(=O)n(C2CCC(N3CCOC3=O)C2)c2c1cnn1cc(C3CC3)c(-c3cc4c(s3)CCOC4)c21] |
| I-183 | 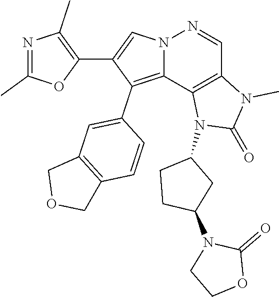 |
| I-183′ | 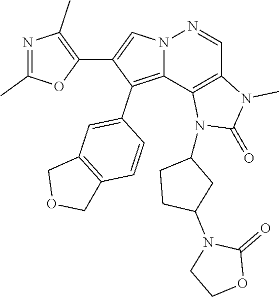 |
| I-184 | 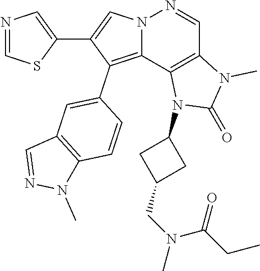 [SMILES: CCC(=O)N(C)C[C@H]1C[C@H](n2c(=O)n(C)c3cnn4cc(-c5cncs5)c(-c5ccc6c(cnn6C)c5)c4c32)C1] |
| I-184′ | 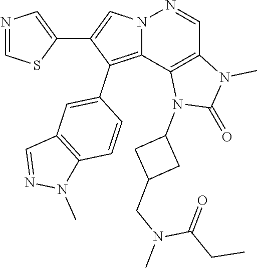 [SMILES: CCC(=O)N(C)CC1CC(n2c(=O)n(C)c3cnn4cc(-c5cncs5)c(-c5ccc6c(cnn6C)c5)c4c32)C1] |
| I-185 | 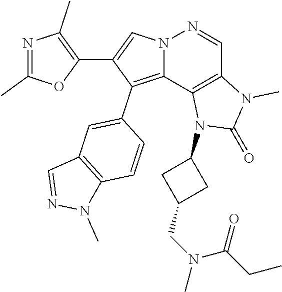 [SMILES: CCC(=O)N(C)C[C@H]1C[C@H](n2c(=O)n(C)c3cnn4cc(-c5oc(C)nc5C)c(-c5ccc6c(cnn6C)c5)c4c32)C1] |
| I-185′ | 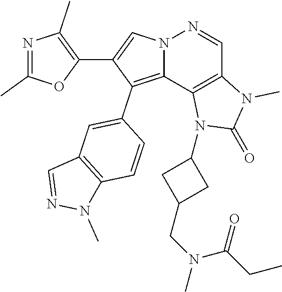 [SMILES: CCC(=O)N(C)CC1CC(n2c(=O)n(C)c3cnn4cc(-c5oc(C)nc5C)c(-c5ccc6c(cnn6C)c5)c4c32)C1] |
| I-186 | 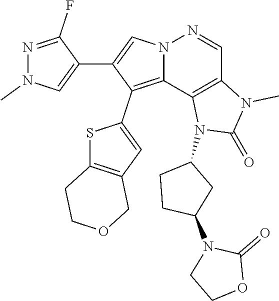 |
| I-186′ | 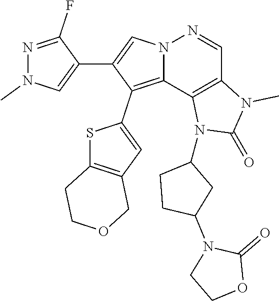 |
| I-187 | 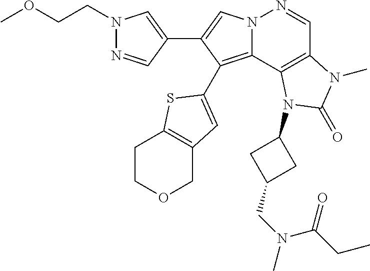 [SMILES: CCC(=O)N(C)C[C@H]1C[C@H](n2c(=O)n(C)c3cnn4cc(-c5cnn(CCOC)c5)c(-c5cc6c(s5)CCOC6)c4c32)C1] |
| I-187′ | 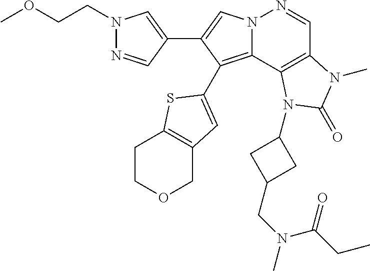 [SMILES: CCC(=O)N(C)CC1CC(n2c(=O)n(C)c3cnn4cc(-c5cnn(CCOC)c5)c(-c5cc6c(s5)CCOC6)c4c32)C1] |
| I-188 | 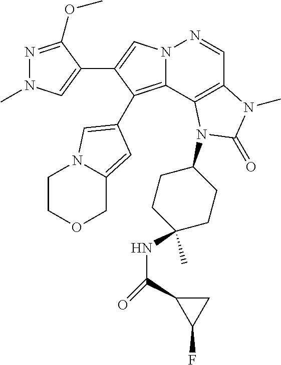 |
| I-188′ | 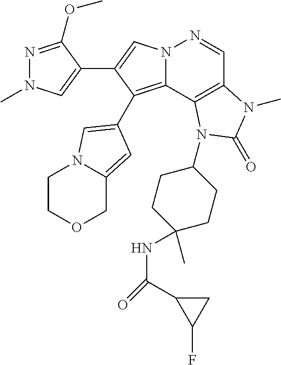 |
| I-189 | 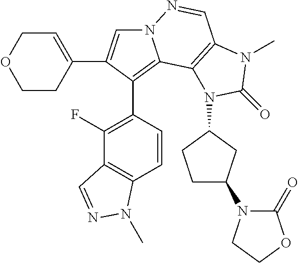 [SMILES: Cn1ncc2c(F)c(-c3c(C4=CCOCC4)cn4ncc5c(c34)n([C@H]3CC[C@H](N4CCOC4=O)C3)c(=O)n5C)ccc21] |
| I-189′ | 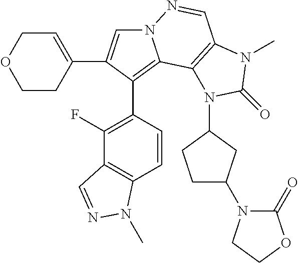 [SMILES: Cn1ncc2c(F)c(-c3c(C4=CCOCC4)cn4ncc5c(c34)n(C3CCC(N4CCOC4=O)C3)c(=O)n5C)ccc21] |
| I-190 | 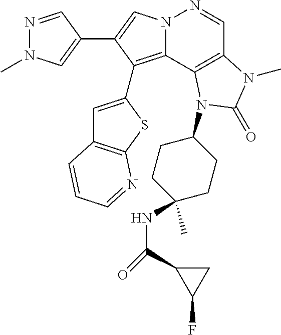 |
| I-190′ | 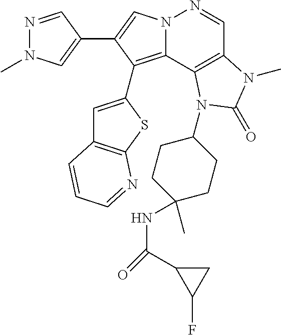 |
| I-191 | 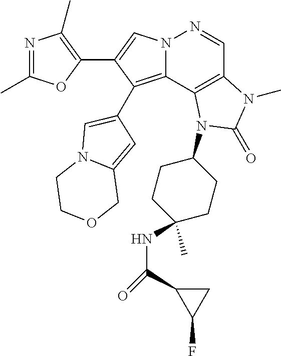 |
| I-191′ | 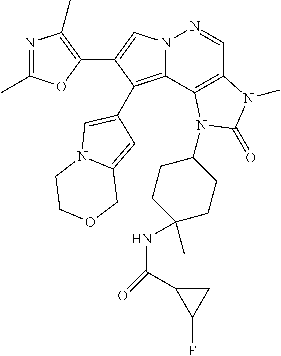 |
| I-192 | 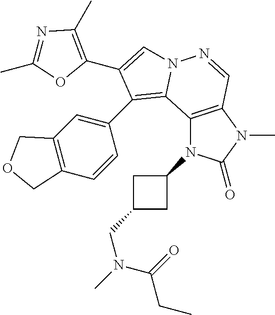 |
| I-192′ | 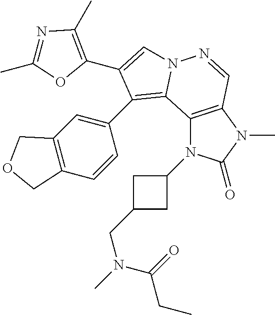 |
| I-193 | 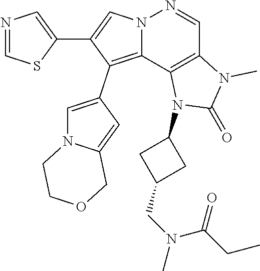 [SMILES: CCC(=O)N(C)C[C@H]1C[C@H](n2c(=O)n(C)c3cnn4cc(-c5cncs5)c(-c5cc6n(c5)CCOC6)c4c32)C1] |
| I-193′ | 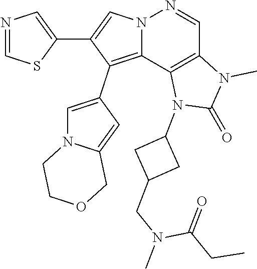 [SMILES: CCC(=O)N(C)CC1CC(n2c(=O)n(C)c3cnn4cc(-c5cncs5)c(-c5cc6n(c5)CCOC6)c4c32)C1] |
| I-194 | 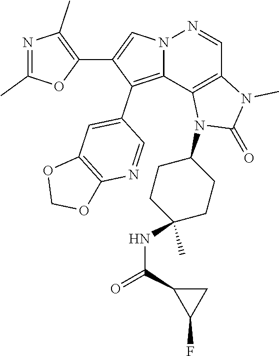 |
| I-194′ | 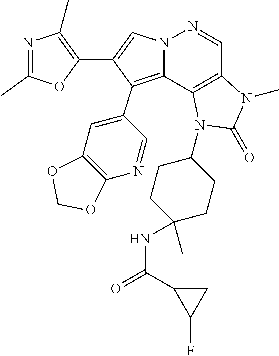 |
| I-195 | 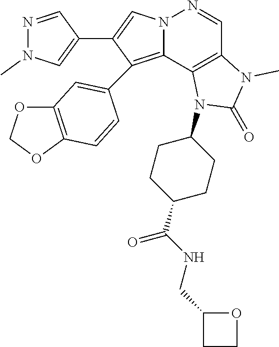 |
| I-195′ | 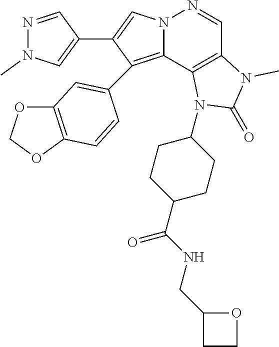 |
| I-196 | 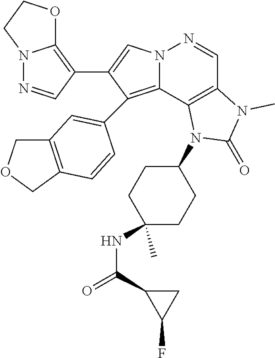 [SMILES: Cn1c(=O)n([C@H]2CC[C@@](C)(NC(=O)[C@H]3C[C@H]3F)CC2)c2c1cnn1cc(-c3cnn4c3OCC4)c(-c3ccc4c(c3)COC4)c21] |
| I-196′ | 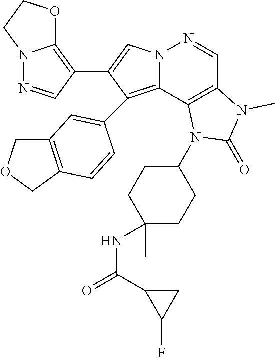 [SMILES: Cn1c(=O)n(C2CCC(C)(NC(=O)C3CC3F)CC2)c2c1cnn1cc(-c3cnn4c3OCC4)c(-c3ccc4c(c3)COC4)c21] |
| I-197 | 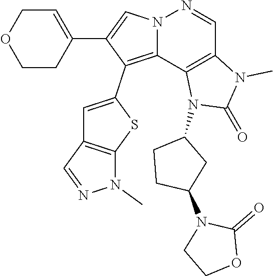 [SMILES: Cn1ncc2cc(-c3c(C4=CCOCC4)cn4ncc5c(c34)n([C@H]3CC[C@H](N4CCOC4=O)C3)c(=O)n5C)sc21] |
| I-197′ | 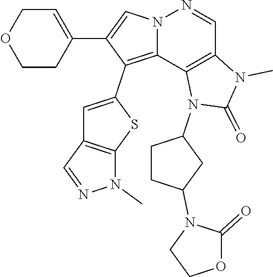 [SMILES: Cn1ncc2cc(-c3c(C4=CCOCC4)cn4ncc5c(c34)n(C3CCC(N4CCOC4=O)C3)c(=O)n5C)sc21] |
| I-198 | 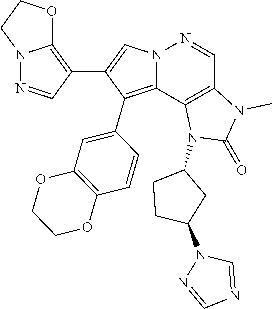 [SMILES: Cn1c(=O)n([C@H]2CC[C@H](n3cncn3)C2)c2c1cnn1cc(-c3cnn4c3OCC4)c(-c3ccc4c(c3)OCCO4)c21] |
| I-198′ | 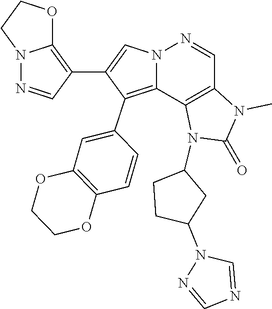 [SMILES: Cn1c(=O)n(C2CCC(n3cncn3)C2)c2c1cnn1cc(-c3cnn4c3OCC4)c(-c3ccc4c(c3)OCCO4)c21] |
| I-199 | 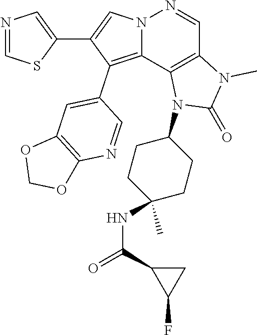 [SMILES: Cn1c(=O)n([C@H]2CC[C@@](C)(NC(=O)[C@H]3C[C@H]3F)CC2)c2c1cnn1cc(-c3cncs3)c(-c3cnc4c(c3)OCO4)c21] |
| I-199′ | 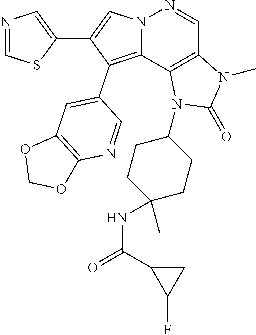 [SMILES: Cn1c(=O)n(C2CCC(C)(NC(=O)C3CC3F)CC2)c2c1cnn1cc(-c3cncs3)c(-c3cnc4c(c3)OCO4)c21] |
| I-200 | 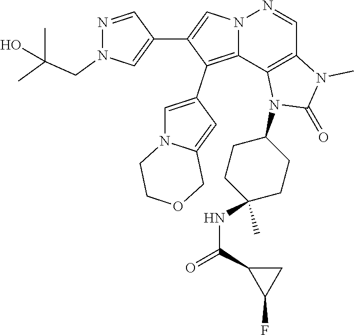 [SMILES: Cn1c(=O)n([C@H]2CC[C@@](C)(NC(=O)[C@H]3C[C@H]3F)CC2)c2c1cnn1cc(-c3cnn(CC(C)(C)O)c3)c(-c3cc4n(c3)CCOC4)c21] |
| I-200′ | 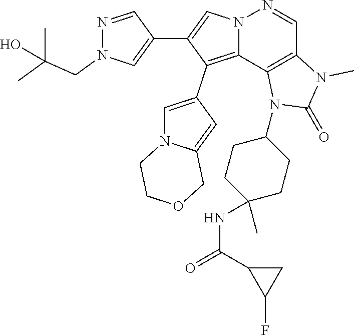 [SMILES: Cn1c(=O)n(C2CCC(C)(NC(=O)C3CC3F)CC2)c2c1cnn1cc(-c3cnn(CC(C)(C)O)c3)c(-c3cc4n(c3)CCOC4)c21] |
| I-201 | 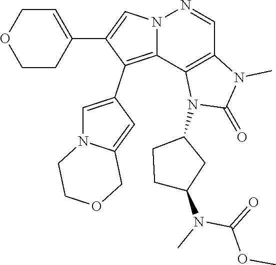 [SMILES: COC(=O)N(C)[C@H]1CC[C@H](n2c(=O)n(C)c3cnn4cc(C5=CCOCC5)c(-c5cc6n(c5)CCOC6)c4c32)C1] |
| I-201′ | 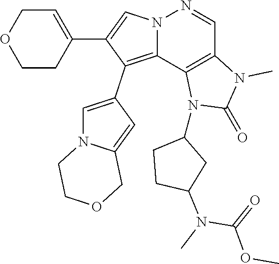 [SMILES: COC(=O)N(C)C1CCC(n2c(=O)n(C)c3cnn4cc(C5=CCOCC5)c(-c5cc6n(c5)CCOC6)c4c32)C1] |
| I-202 | 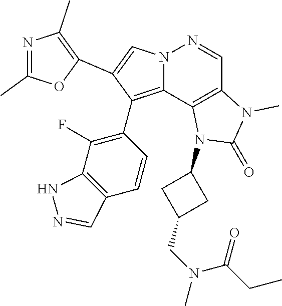 [SMILES: CCC(=O)N(C)C[C@H]1C[C@H](n2c(=O)n(C)c3cnn4cc(-c5oc(C)nc5C)c(-c5ccc6cn[nH]c6c5F)c4c32)C1] |
| I-202′ | 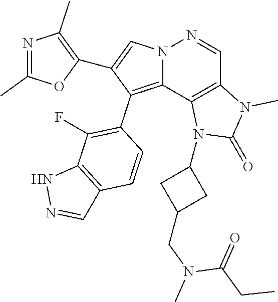 |
| I-203 | 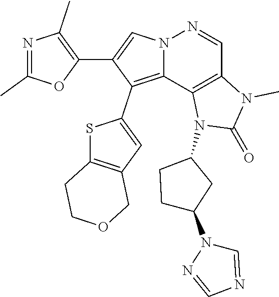 |
| I-203′ | 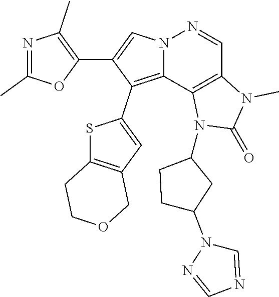 |
| I-204 | 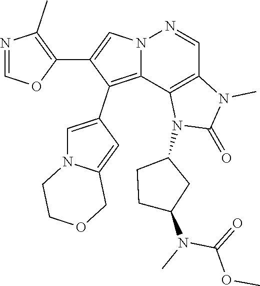 [SMILES: COC(=O)N(C)[C@H]1CC[C@H](n2c(=O)n(C)c3cnn4cc(-c5ocnc5C)c(-c5cc6n(c5)CCOC6)c4c32)C1] |
| I-204′ | 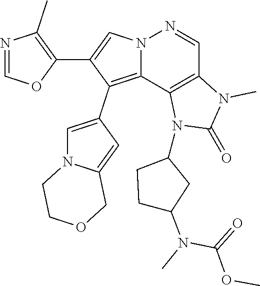 [SMILES: COC(=O)N(C)C1CCC(n2c(=O)n(C)c3cnn4cc(-c5ocnc5C)c(-c5cc6n(c5)CCOC6)c4c32)C1] |
| I-205 | 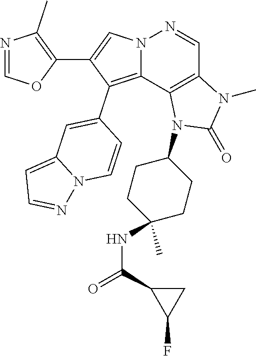 [SMILES: Cc1ncoc1-c1cn2ncc3c(c2c1-c1ccn2nccc2c1)n([C@H]1CC[C@@](C)(NC(=O)[C@H]2C[C@H]2F)CC1)c(=O)n3C] |
| I-205′ | 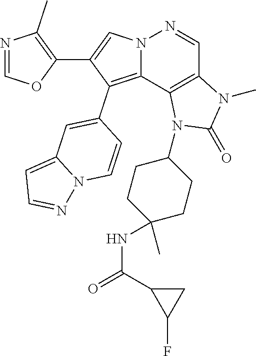 [SMILES: Cc1ncoc1-c1cn2ncc3c(c2c1-c1ccn2nccc2c1)n(C1CCC(C)(NC(=O)C2CC2F)CC1)c(=O)n3C] |
| I-206 | 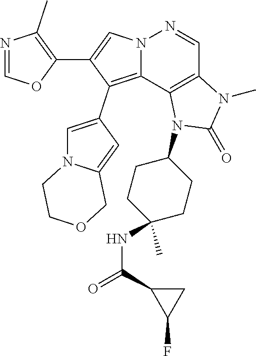 [SMILES: Cc1ncoc1-c1cn2ncc3c(c2c1-c1cc2n(c1)CCOC2)n([C@H]1CC[C@@](C)(NC(=O)[C@H]2C[C@H]2F)CC1)c(=O)n3C] |
| I-206′ | 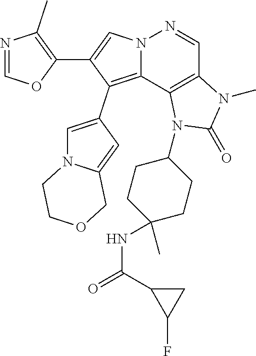 [SMILES: Cc1ncoc1-c1cn2ncc3c(c2c1-c1cc2n(c1)CCOC2)n(C1CCC(C)(NC(=O)C2CC2F)CC1)c(=O)n3C] |
| I-207 | 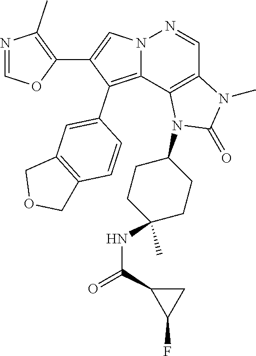 [SMILES: Cc1ncoc1-c1cn2ncc3c(c2c1-c1ccc2c(c1)COC2)n([C@H]1CC[C@@](C)(NC(=O)[C@H]2C[C@H]2F)CC1)c(=O)n3C] |
| I-207′ | 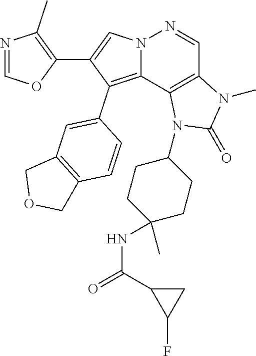 [SMILES: Cc1ncoc1-c1cn2ncc3c(c2c1-c1ccc2c(c1)COC2)n(C1CCC(C)(NC(=O)C2CC2F)CC1)c(=O)n3C] |
| I-208 | 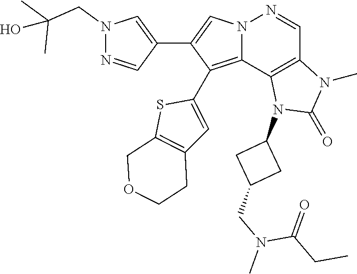 [SMILES: CCC(=O)N(C)C[C@H]1C[C@H](n2c(=O)n(C)c3cnn4cc(-c5cnn(CC(C)(C)O)c5)c(-c5cc6c(s5)COCC6)c4c32)C1] |
| I-208′ | 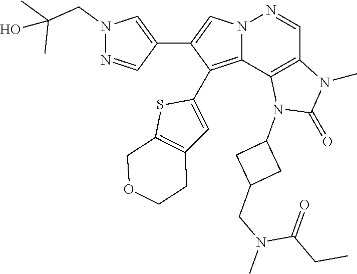 [SMILES: CCC(=O)N(C)CC1CC(n2c(=O)n(C)c3cnn4cc(-c5cnn(CC(C)(C)O)c5)c(-c5cc6c(s5)COCC6)c4c32)C1] |
| I-209 | 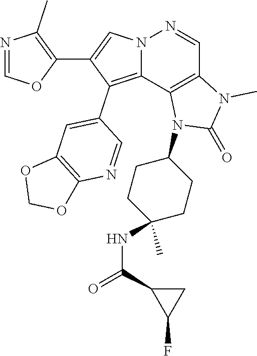 [SMILES: Cc1ncoc1-c1cn2ncc3c(c2c1-c1cnc2c(c1)OCO2)n([C@H]1CC[C@@](C)(NC(=O)[C@H]2C[C@H]2F)CC1)c(=O)n3C] |
| I-209′ | 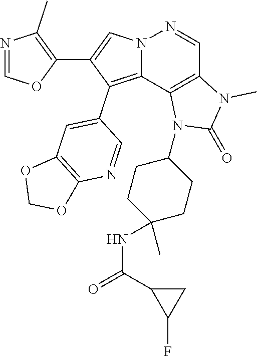 [SMILES: Cc1ncoc1-c1cn2ncc3c(c2c1-c1cnc2c(c1)OCO2)n(C1CCC(C)(NC(=O)C2CC2F)CC1)c(=O)n3C] |
| I-210 | 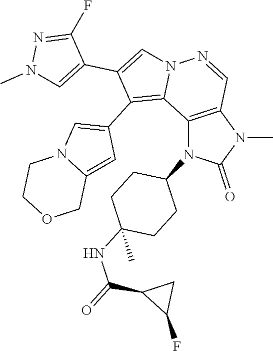 |
| I-210′ | 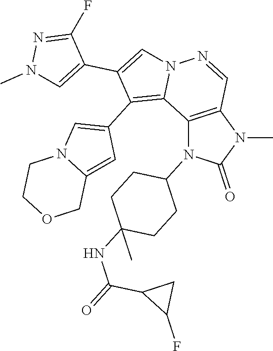 |
| I-211 | 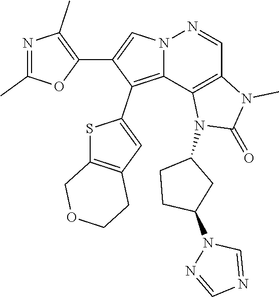 |
| I-211′ | 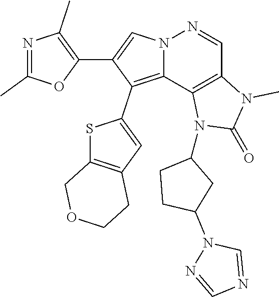 |
| I-212 | 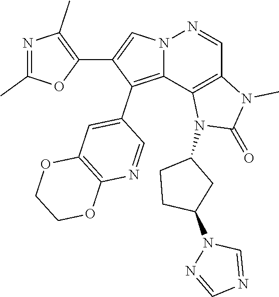 |
| I-212′ | 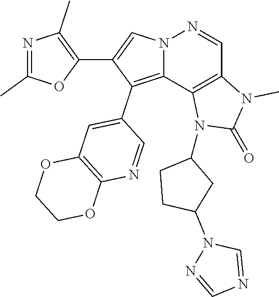 |
| I-213 | 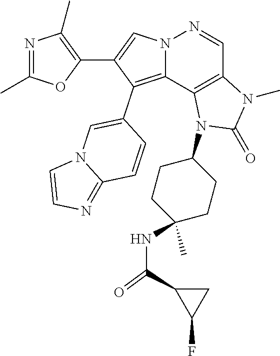 |
| I-213′ | 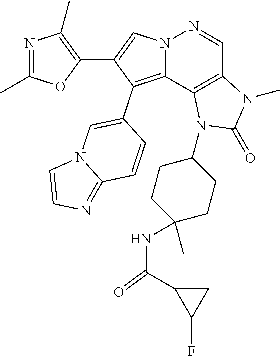 |
| I-214 | 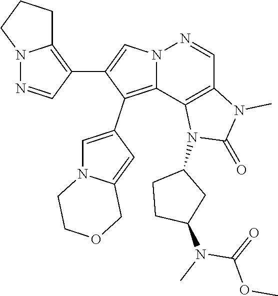 [SMILES: COC(=O)N(C)[C@H]1CC[C@H](n2c(=O)n(C)c3cnn4cc(-c5cnn6c5CCC6)c(-c5cc6n(c5)CCOC6)c4c32)C1] |
| I-214′ | 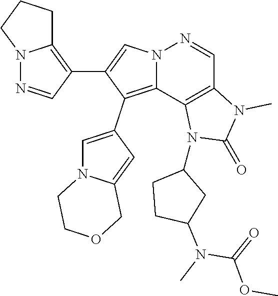 [SMILES: COC(=O)N(C)C1CCC(n2c(=O)n(C)c3cnn4cc(-c5cnn6c5CCC6)c(-c5cc6n(c5)CCOC6)c4c32)C1] |
| I-215 | 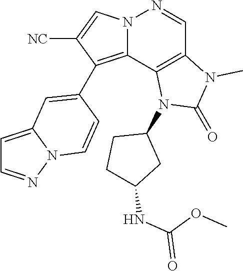 [SMILES: COC(=O)N[C@@H]1CC[C@@H](n2c(=O)n(C)c3cnn4cc(C#N)c(-c5ccn6nccc6c5)c4c32)C1] |
| I-215′ | 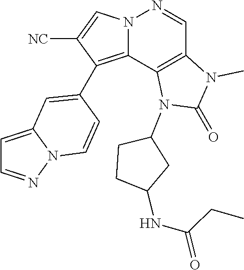 [SMILES: CCC(=O)NC1CCC(n2c(=O)n(C)c3cnn4cc(C#N)c(-c5ccn6nccc6c5)c4c32)C1] |
| I-216 | 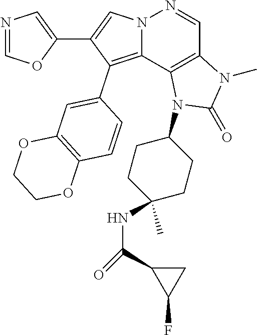 [SMILES: Cn1c(=O)n([C@H]2CC[C@@](C)(NC(=O)[C@H]3C[C@H]3F)CC2)c2c1cnn1cc(-c3cnco3)c(-c3ccc4c(c3)OCCO4)c21] |
| I-216′ | 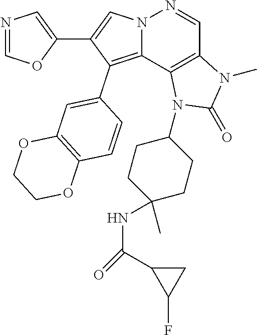 [SMILES: Cn1c(=O)n(C2CCC(C)(NC(=O)C3CC3F)CC2)c2c1cnn1cc(-c3cnco3)c(-c3ccc4c(c3)OCCO4)c21] |
| I-217 | 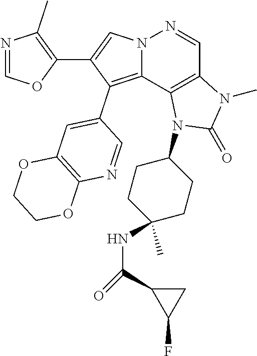 [SMILES: Cc1ncoc1-c1cn2ncc3c(c2c1-c1cnc2c(c1)OCCO2)n([C@H]1CC[C@@](C)(NC(=O)[C@H]2C[C@H]2F)CC1)c(=O)n3C] |
| I-217′ | 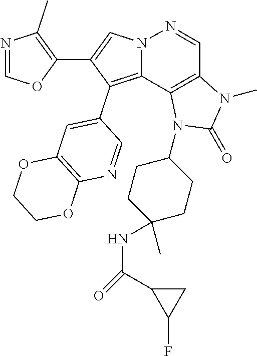 [SMILES: Cc1ncoc1-c1cn2ncc3c(c2c1-c1cnc2c(c1)OCCO2)n(C1CCC(C)(NC(=O)C2CC2F)CC1)c(=O)n3C] |
| I-218 | 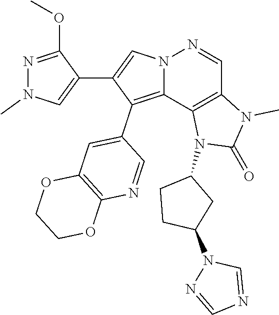 |
| I-218′ | 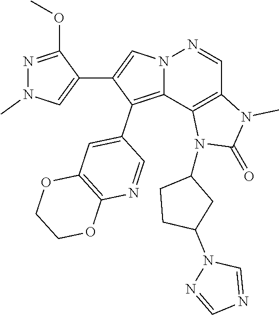 |
| I-219 | 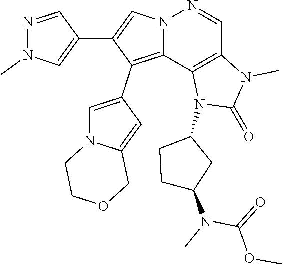 |
| I-219′ | 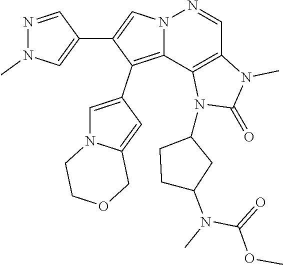 |
| I-220 | 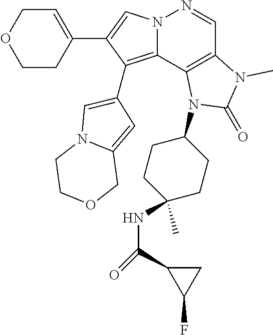 [SMILES: Cn1c(=O)n([C@H]2CC[C@@](C)(NC(=O)[C@H]3C[C@H]3F)CC2)c2c1cnn1cc(C3=CCOCC3)c(-c3cc4n(c3)CCOC4)c21] |
| I-220′ | 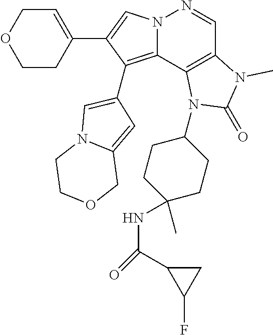 [SMILES: Cn1c(=O)n(C2CCC(C)(NC(=O)C3CC3F)CC2)c2c1cnn1cc(C3=CCOCC3)c(-c3cc4n(c3)CCOC4)c21] |
| I-221 | 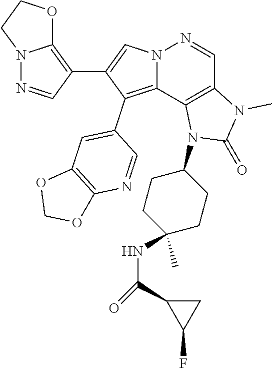 [SMILES: Cn1c(=O)n([C@H]2CC[C@@](C)(NC(=O)[C@H]3C[C@H]3F)CC2)c2c1cnn1cc(-c3cnn4c3OCC4)c(-c3cnc4c(c3)OCO4)c21] |
| I-221′ | 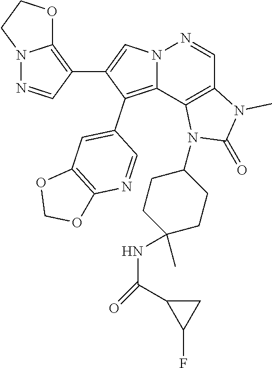 [SMILES: Cn1c(=O)n(C2CCC(C)(NC(=O)C3CC3F)CC2)c2c1cnn1cc(-c3cnn4c3OCC4)c(-c3cnc4c(c3)OCO4)c21] |
| I-222 | 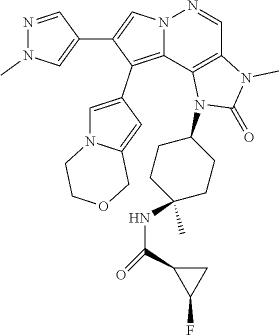 |
| I-222′ | 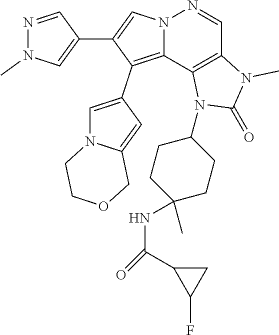 |
| I-223 | 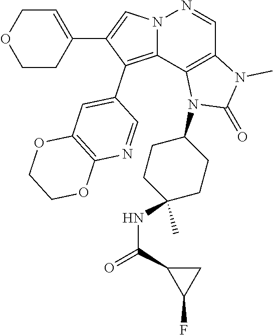 [SMILES: Cn1c(=O)n([C@H]2CC[C@@](C)(NC(=O)[C@H]3C[C@H]3F)CC2)c2c1cnn1cc(C3=CCOCC3)c(-c3cnc4c(c3)OCCO4)c21] |
| I-223′ | 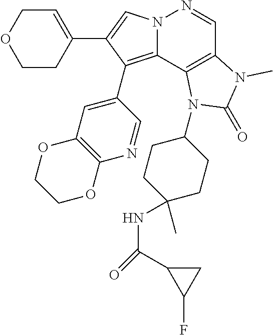 [SMILES: Cn1c(=O)n(C2CCC(C)(NC(=O)C3CC3F)CC2)c2c1cnn1cc(C3=CCOCC3)c(-c3cnc4c(c3)OCCO4)c21] |
| I-224 | 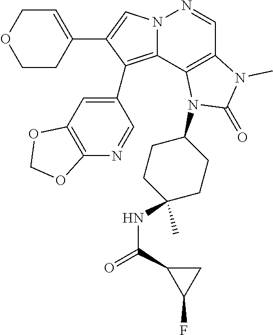 [SMILES: Cn1c(=O)n([C@H]2CC[C@@](C)(NC(=O)[C@H]3C[C@H]3F)CC2)c2c1cnn1cc(C3=CCOCC3)c(-c3cnc4c(c3)OCO4)c21] |
| I-224′ | 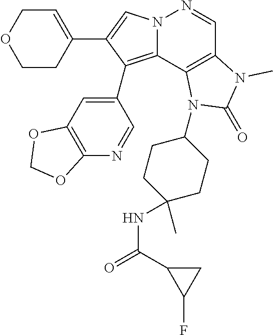 [SMILES: Cn1c(=O)n(C2CCC(C)(NC(=O)C3CC3F)CC2)c2c1cnn1cc(C3=CCOCC3)c(-c3cnc4c(c3)OCO4)c21] |
| I-225 | 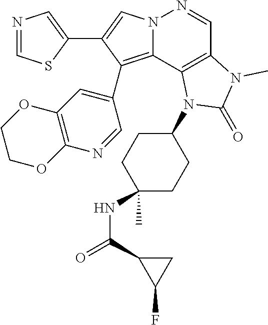 [SMILES: Cn1c(=O)n([C@H]2CC[C@@](C)(NC(=O)[C@H]3C[C@H]3F)CC2)c2c1cnn1cc(-c3cncs3)c(-c3cnc4c(c3)OCCO4)c21] |
| I-225′ | 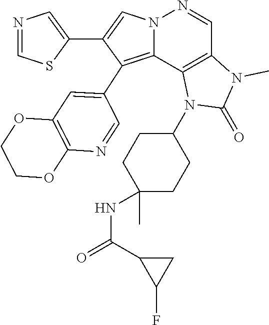 [SMILES: Cn1c(=O)n(C2CCC(C)(NC(=O)C3CC3F)CC2)c2c1cnn1cc(-c3cncs3)c(-c3cnc4c(c3)OCCO4)c21] |
| I-226 | 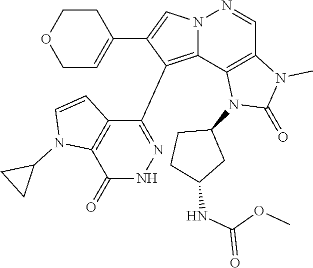 [SMILES: COC(=O)N[C@@H]1CC[C@@H](n2c(=O)n(C)c3cnn4cc(C5=CCOCC5)c(-c5n[nH]c(=O)c6c5ccn6C5CC5)c4c32)C1] |
| I-226′ | 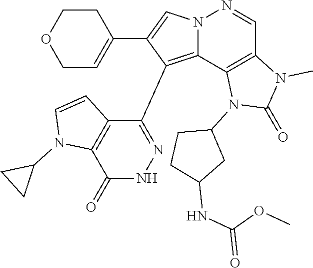 [SMILES: COC(=O)NC1CCC(n2c(=O)n(C)c3cnn4cc(C5=CCOCC5)c(-c5n[nH]c(=O)c6c5ccn6C5CC5)c4c32)C1] |
| I-227 | 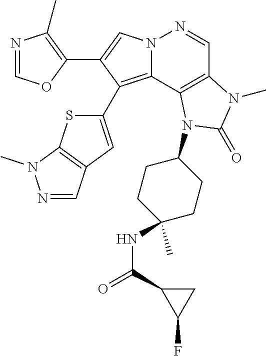 |
| I-227′ | 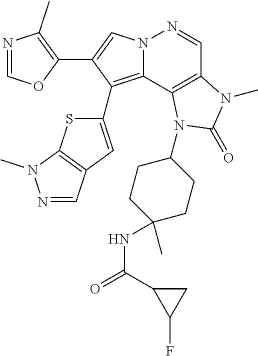 |
| I-228 | 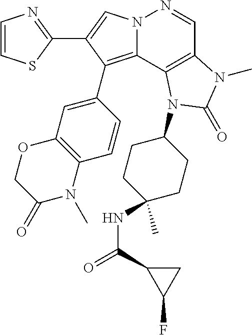 [SMILES: CN1C(=O)COc2cc(-c3c(-c4nccs4)cn4ncc5c(c34)n([C@H]3CC[C@@](C)(NC(=O)[C@H]4C[C@H]4F)CC3)c(=O)n5C)ccc21] |
| I-228′ | 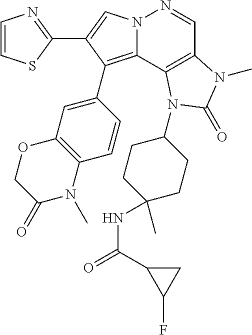 [SMILES: CN1C(=O)COc2cc(-c3c(-c4nccs4)cn4ncc5c(c34)n(C3CCC(C)(NC(=O)C4CC4F)CC3)c(=O)n5C)ccc21] |
| I-229 | 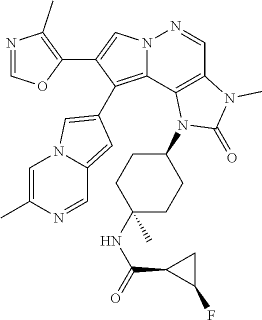 |
| I-229′ | 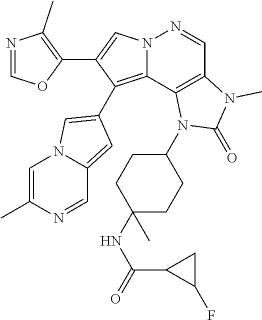 |
| I-230 | 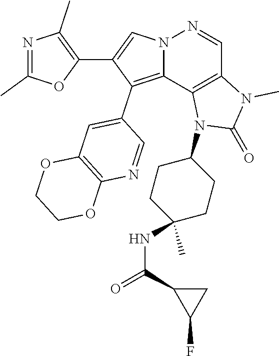 |
| I-230′ | 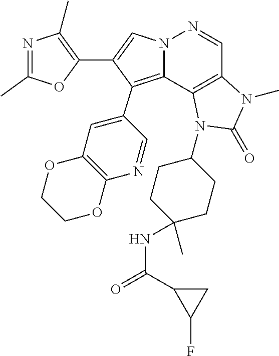 |
| I-231 | 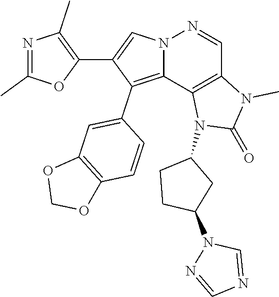 |
| I-231′ | 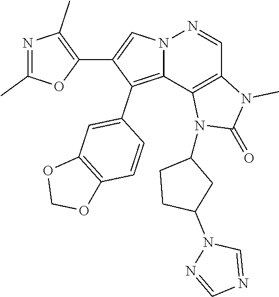 |
| I-232 | 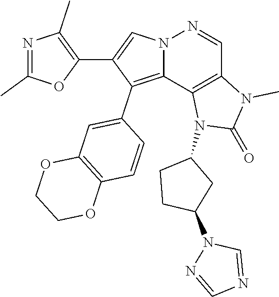 |
| I-232′ | 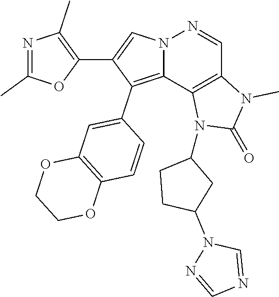 |
| I-233 | 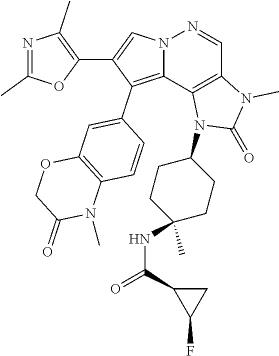 |
| I-233′ | 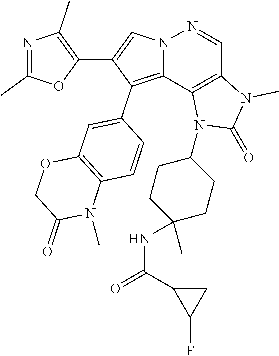 |
| I-234 | 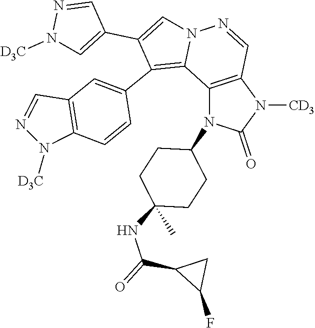 [SMILES: [2H]C([2H])([2H])n1cc(-c2cn3ncc4c(c3c2-c2ccc3c(cnn3C([2H])([2H])[2H])c2)n([C@H]2CC[C@@](C)(NC(=O)[C@H]3C[C@H]3F)CC2)c(=O)n4C([2H])([2H])[2H])cn1] |
| I-234′ | 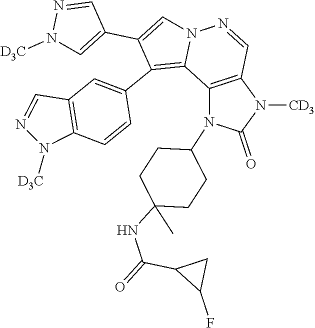 [SMILES: [2H]C([2H])([2H])n1cc(-c2cn3ncc4c(c3c2-c2ccc3c(cnn3C([2H])([2H])[2H])c2)n(C2CCC(C)(NC(=O)C3CC3F)CC2)c(=O)n4C([2H])([2H])[2H])cn1] |
| I-235 | 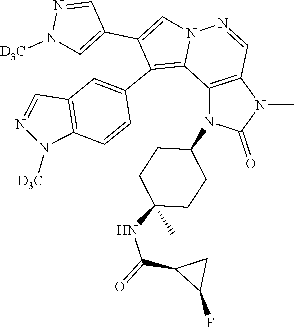 [SMILES: [2H]C([2H])([2H])n1cc(-c2cn3ncc4c(c3c2-c2ccc3c(cnn3C([2H])([2H])[2H])c2)n([C@H]2CC[C@@](C)(NC(=O)[C@H]3C[C@H]3F)CC2)c(=O)n4C)cn1] |
| I-235′ | 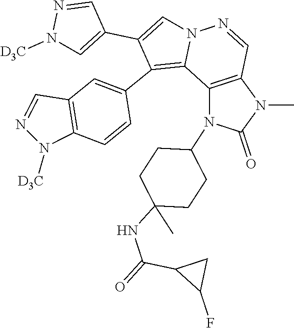 [SMILES: [2H]C([2H])([2H])n1cc(-c2cn3ncc4c(c3c2-c2ccc3c(cnn3C([2H])([2H])[2H])c2)n(C2CCC(C)(NC(=O)C3CC3F)CC2)c(=O)n4C)cn1] |
| I-236 | 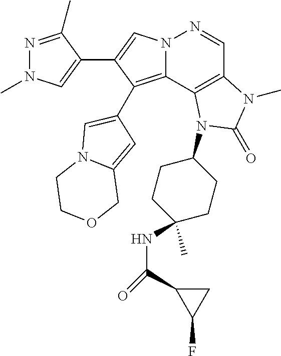 |
| I-236′ | 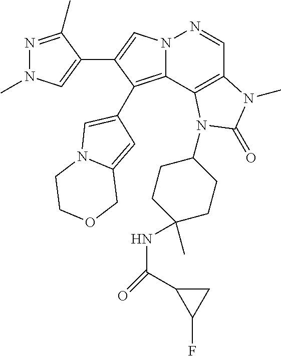 |
| I-237 | 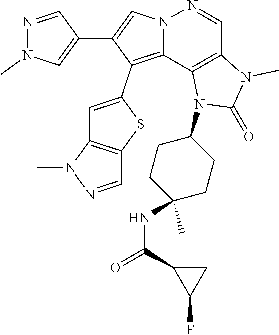 |
| I-237′ | 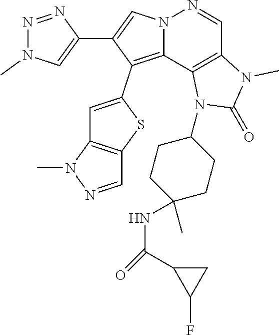 |
| I-238 | 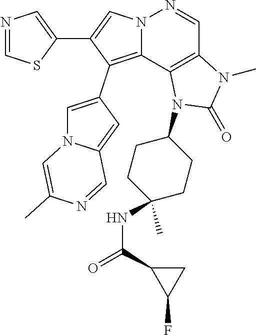 [SMILES: Cc1cn2cc(-c3c(-c4cncs4)cn4ncc5c(c34)n([C@H]3CC[C@@](C)(NC(=O)[C@H]4C[C@H]4F)CC3)c(=O)n5C)cc2cn1] |
| I-238′ | 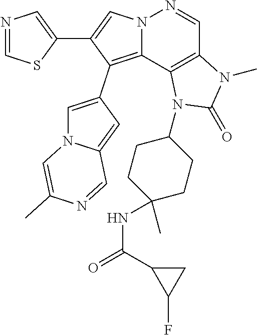 [SMILES: Cc1cn2cc(-c3c(-c4cncs4)cn4ncc5c(c34)n(C3CCC(C)(NC(=O)C4CC4F)CC3)c(=O)n5C)cc2cn1] |
| I-239 | 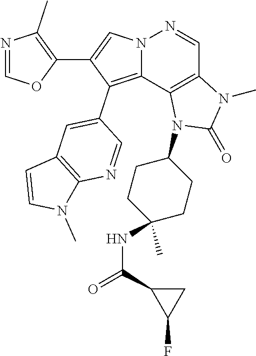 [SMILES: Cc1ncoc1-c1cn2ncc3c(c2c1-c1cnc2c(ccn2C)c1)n([C@H]1CC[C@@](C)(NC(=O)[C@H]2C[C@H]2F)CC1)c(=O)n3C] |
| I-239′ | 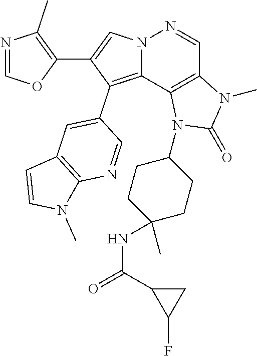 [SMILES: Cc1ncoc1-c1cn2ncc3c(c2c1-c1cnc2c(ccn2C)c1)n(C1CCC(C)(NC(=O)C2CC2F)CC1)c(=O)n3C] |
| I-240 | 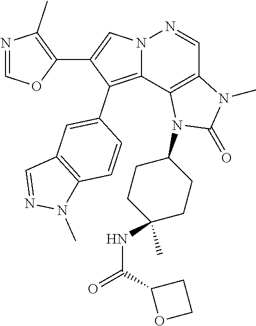 [SMILES: Cc1ncoc1-c1cn2ncc3c(c2c1-c1ccc2c(cnn2C)c1)n([C@H]1CC[C@@](C)(NC(=O)[C@@H]2CCO2)CC1)c(=O)n3C] |
| I-240′ | 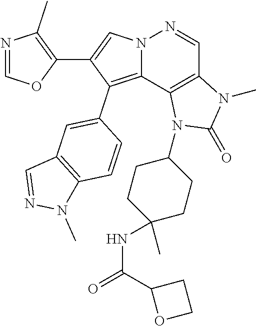 [SMILES: Cc1ncoc1-c1cn2ncc3c(c2c1-c1ccc2c(cnn2C)c1)n(C1CCC(C)(NC(=O)C2CCO2)CC1)c(=O)n3C] |
| I-241 | 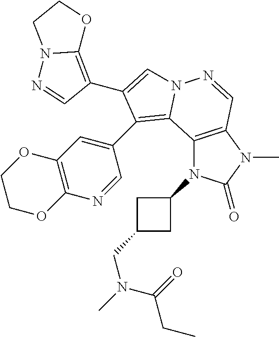 [SMILES: CCC(=O)N(C)C[C@H]1C[C@H](n2c(=O)n(C)c3cnn4cc(-c5cnn6c5OCC6)c(-c5cnc6c(c5)OCCO6)c4c32)C1] |
| I-241′ | 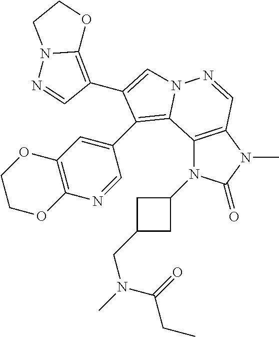 [SMILES: CCC(=O)N(C)CC1CC(n2c(=O)n(C)c3cnn4cc(-c5cnn6c5OCC6)c(-c5cnc6c(c5)OCCO6)c4c32)C1] |
| I-242 | 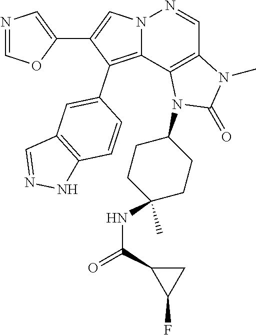 [SMILES: Cn1c(=O)n([C@H]2CC[C@@](C)(NC(=O)[C@H]3C[C@H]3F)CC2)c2c1cnn1cc(-c3cnco3)c(-c3ccc4[nH]ncc4c3)c21] |
| I-242′ | 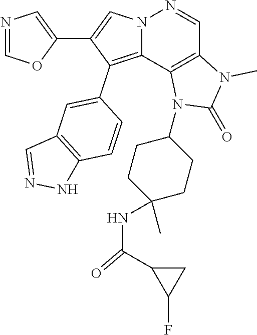 [SMILES: Cn1c(=O)n(C2CCC(C)(NC(=O)C3CC3F)CC2)c2c1cnn1cc(-c3cnco3)c(-c3ccc4[nH]ncc4c3)c21] |
| I-243-1 | 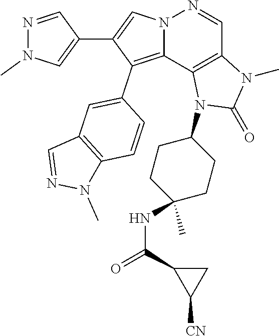 |
| I-243-2 | 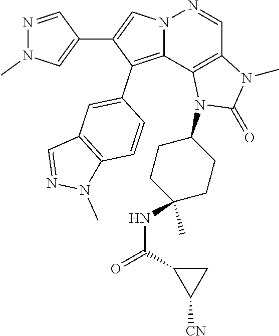 |
| I-243′ | 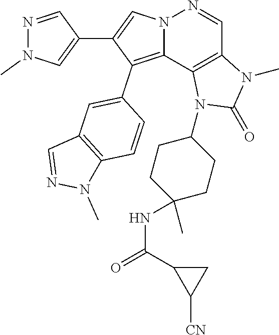 |
| I-244 | 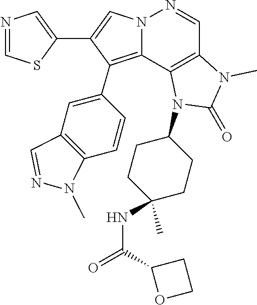 [SMILES: Cn1ncc2cc(-c3c(-c4cncs4)cn4ncc5c(c34)n([C@H]3CC[C@@](C)(NC(=O)[C@@H]4CCO4)CC3)c(=O)n5C)ccc21] |
| I-244′ | 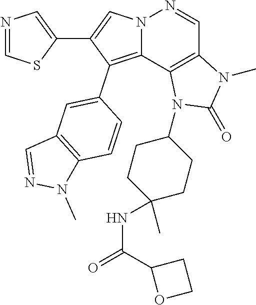 [SMILES: Cn1ncc2cc(-c3c(-c4cncs4)cn4ncc5c(c34)n(C3CCC(C)(NC(=O)C4CCO4)CC3)c(=O)n5C)ccc21] |
| I-245 | 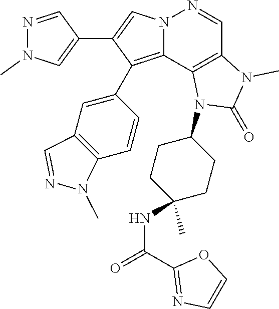 |
| I-245′ | 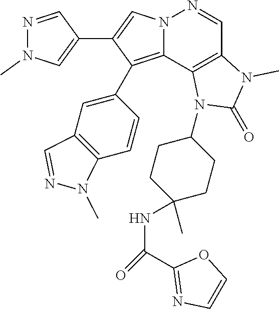 |
| I-246-1 | 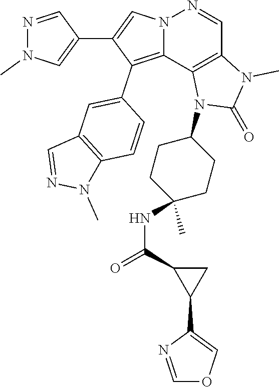 |
| I-246-2 | 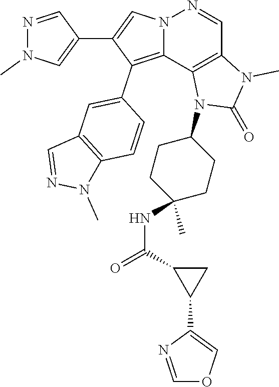 |
| I-246′ | 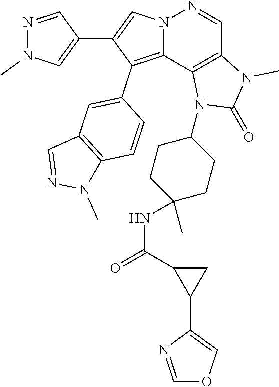 |
| I-247 | 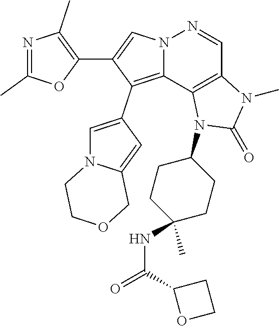 |
| I-247′ | 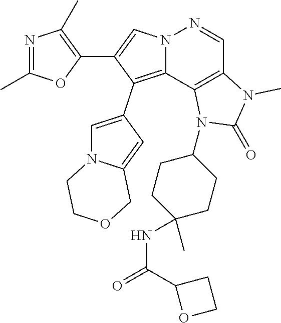 |
| I-248 | 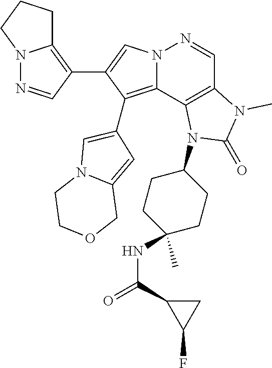 [SMILES: Cn1c(=O)n([C@H]2CC[C@@](C)(NC(=O)[C@H]3C[C@H]3F)CC2)c2c1cnn1cc(-c3cnn4c3CCC4)c(-c3cc4n(c3)CCOC4)c21] |
| I-248′ | 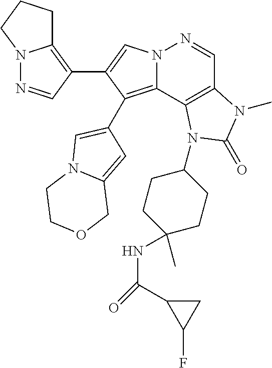 [SMILES: Cn1c(=O)n(C2CCC(C)(NC(=O)C3CC3F)CC2)c2c1cnn1cc(-c3cnn4c3CCC4)c(-c3cc4n(c3)CCOC4)c21] |
| I-249 | 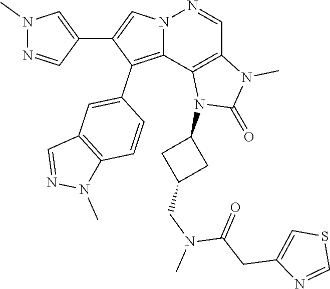 [SMILES: CN(C[C@H]1C[C@H](n2c(=O)n(C)c3cnn4cc(-c5cnn(C)c5)c(-c5ccc6c(cnn6C)c5)c4c32)C1)C(=O)Cc1cscn1] |
| I-249′ | 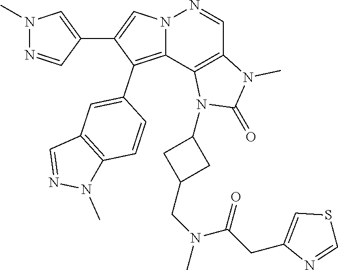 [SMILES: CN(CC1CC(n2c(=O)n(C)c3cnn4cc(-c5cnn(C)c5)c(-c5ccc6c(cnn6C)c5)c4c32)C1)C(=O)Cc1cscn1] |
| I-250 | 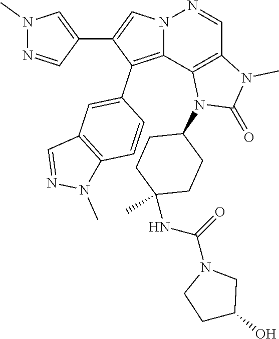 |
| I-250′ | 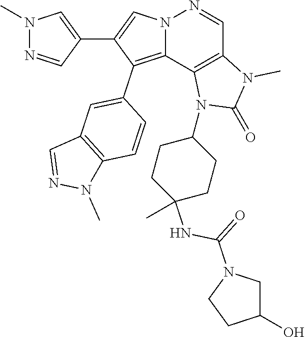 [SMILES: Cn1cc(-c2cn3ncc4c(c3c2-c2ccc3c(cnn3C)c2)n(C2CCC(C)(NC(=O)N3CCC(O)C3)CC2)c(=O)n4C)cn1] |
| I-251 | 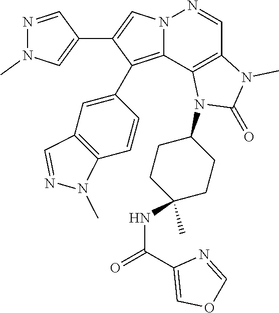 |
| I-251′ | 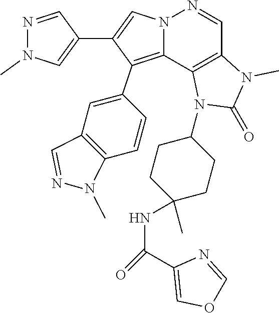 |
| I-252-1 | 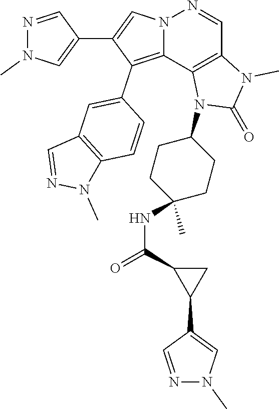 |
| I-252-2 | 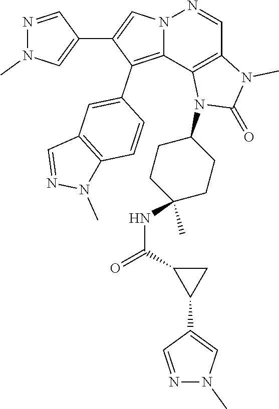 |
| I-252′ | 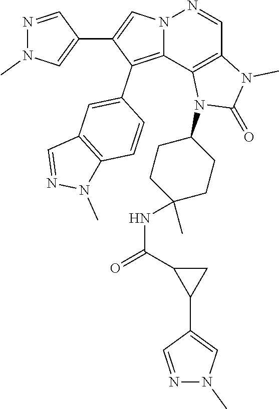 |
| I-253 | 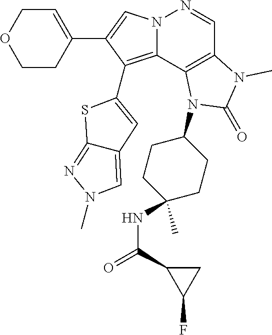 [SMILES: Cn1cc2cc(-c3c(C4=CCOCC4)cn4ncc5c(c34)n([C@H]3CC[C@@](C)(NC(=O)[C@H]4C[C@H]4F)CC3)c(=O)n5C)sc2n1] |
| I-253′ | 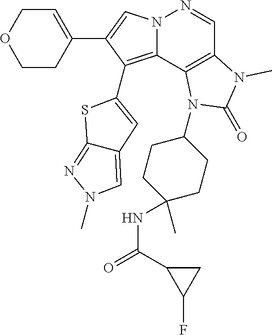 [SMILES: Cn1cc2cc(-c3c(C4=CCOCC4)cn4ncc5c(c34)n(C3CCC(C)(NC(=O)C4CC4F)CC3)c(=O)n5C)sc2n1] |
| I-254 | 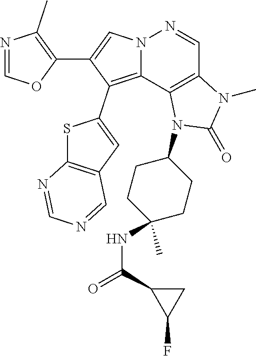 [SMILES: Cc1ncoc1-c1cn2ncc3c(c2c1-c1cc2cncnc2s1)n([C@H]1CC[C@@](C)(NC(=O)[C@H]2C[C@H]2F)CC1)c(=O)n3C] |
| I-254′ | 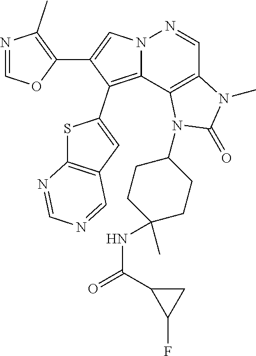 [SMILES: Cc1ncoc1-c1cn2ncc3c(c2c1-c1cc2cncnc2s1)n(C1CCC(C)(NC(=O)C2CC2F)CC1)c(=O)n3C] |
| I-255 | 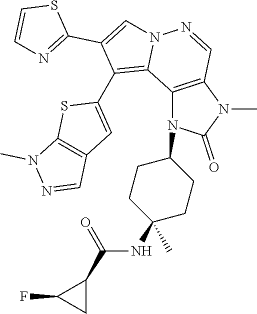 |
| I-255′ | 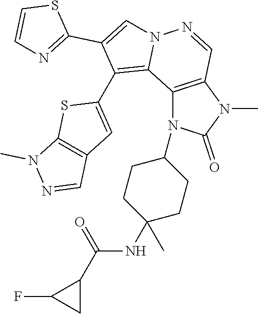 |
| I-256 | 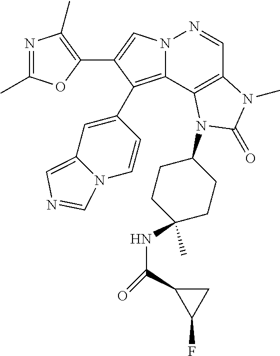 |
| I-256′ | 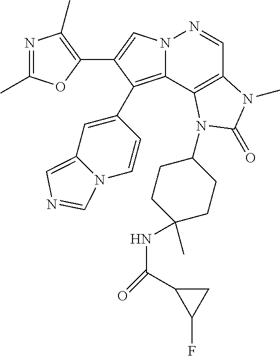 |
| I-257 | 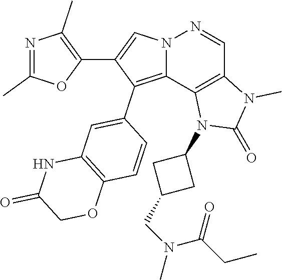 |
| I-257′ | 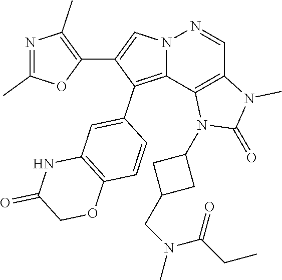 |
| I-258 | 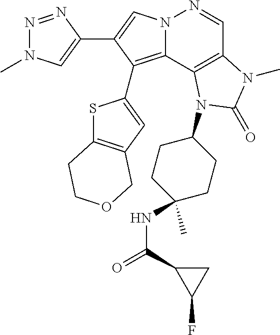 |
| I-258′ | 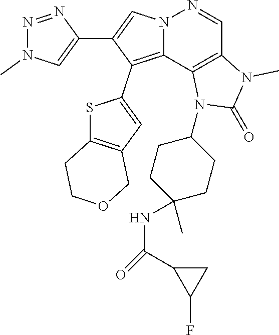 |
| I-259 | 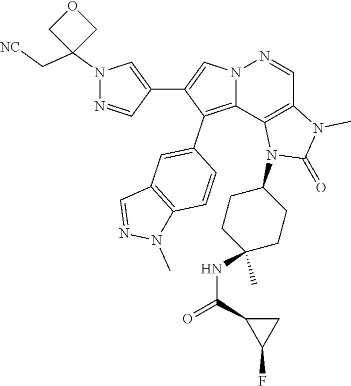 [SMILES: Cn1ncc2cc(-c3c(-c4cnn(C5(CC#N)COC5)c4)cn4ncc5c(c34)n([C@H]3CC[C@@](C)(NC(=O)[C@H]4C[C@H]4F)CC3)c(=O)n5C)ccc21] |
| I-259′ | 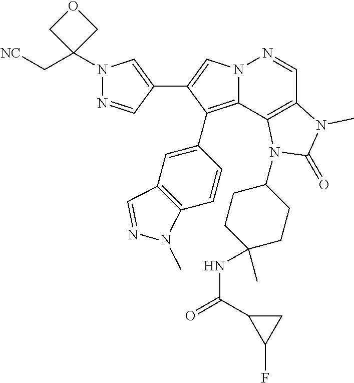 [SMILES: Cn1ncc2cc(-c3c(-c4cnn(C5(CC#N)COC5)c4)cn4ncc5c(c34)n(C3CCC(C)(NC(=O)C4CC4F)CC3)c(=O)n5C)ccc21] |
| I-260 | 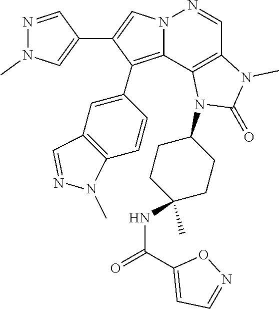 |
| I-260′ | 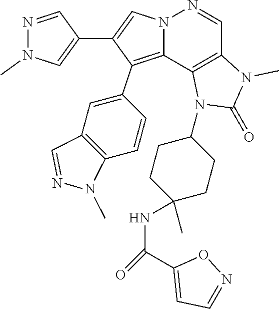 |
| I-261 | 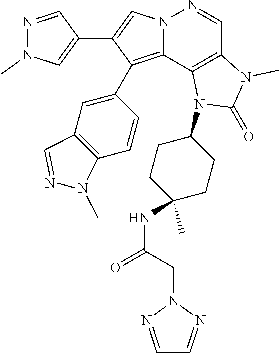 |
| I-261′ | 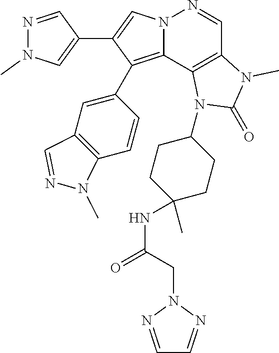 |
| I-262-1 | 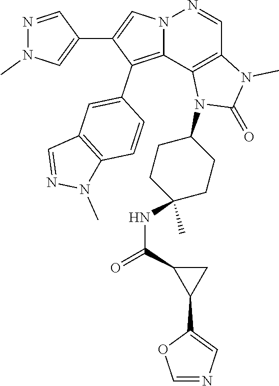 |
| I-262-2 | 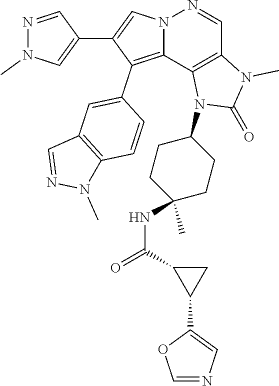 |
| I-262′ | 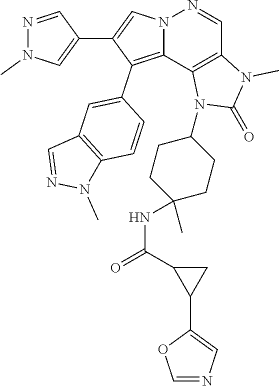 |
| I-263 | 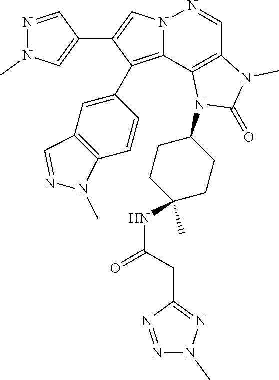 |
| I-263′ | 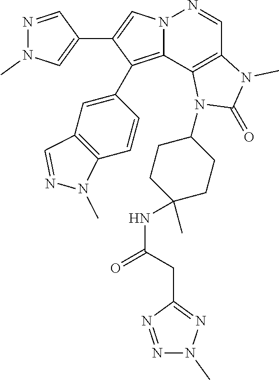 |
| I-264 | 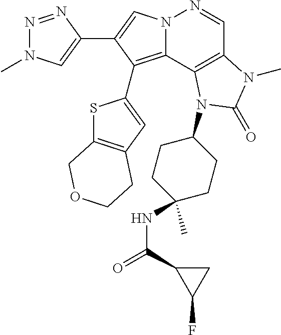 |
| I-264′ | 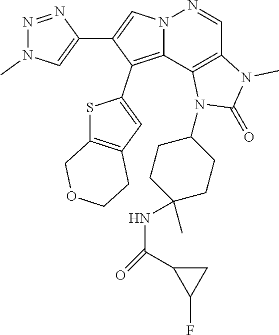 |
| I-265 | 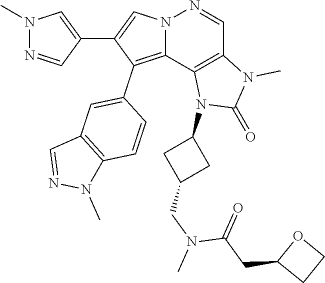 [SMILES: CN(C[C@H]1C[C@H](n2c(=O)n(C)c3cnn4cc(-c5cnn(C)c5)c(-c5ccc6c(cnn6C)c5)c4c32)C1)C(=O)C[C@@H]1CCO1] |
| I-265′ | 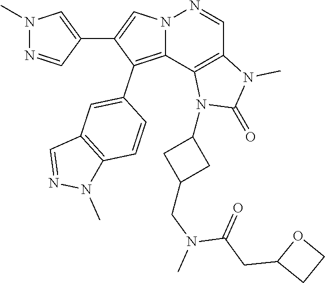 [SMILES: CN(CC1CC(n2c(=O)n(C)c3cnn4cc(-c5cnn(C)c5)c(-c5ccc6c(cnn6C)c5)c4c32)C1)C(=O)CC1CCO1] |
| I-266 | 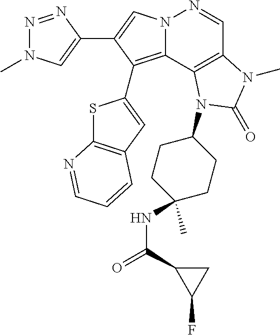 |
| I-266′ | 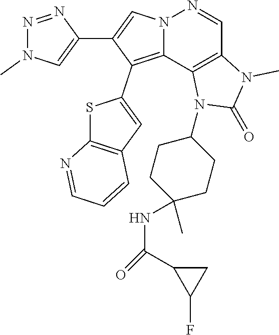 |
| I-267 | 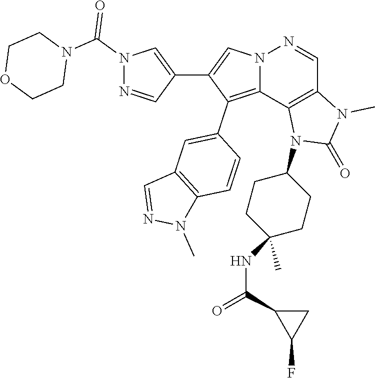 [SMILES: Cn1ncc2cc(-c3c(-c4cnn(C(=O)N5CCOCC5)c4)cn4ncc5c(c34)n([C@H]3CC[C@@](C)(NC(=O)[C@H]4C[C@H]4F)CC3)c(=O)n5C)ccc21] |
| I-267′ | 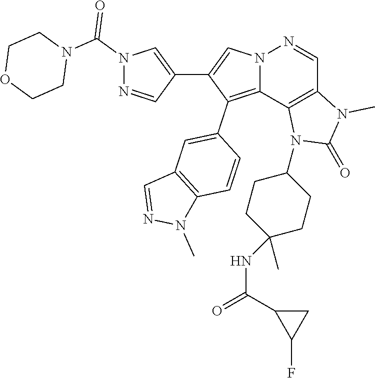 [SMILES: Cn1ncc2cc(-c3c(-c4cnn(C(=O)N5CCOCC5)c4)cn4ncc5c(c34)n(C3CCC(C)(NC(=O)C4CC4F)CC3)c(=O)n5C)ccc21] |
| I-268 | 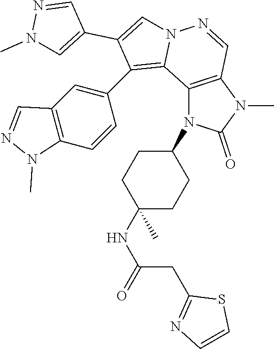 |
| I-268′ | 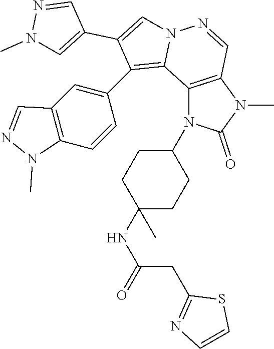 |
| I-269 | 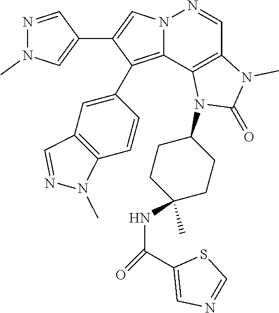 |
| I-269′ | 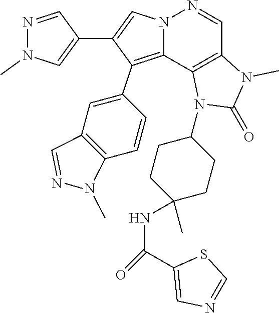 |
| I-270 | 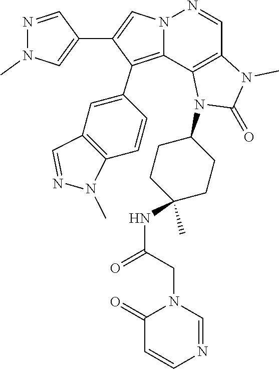 |
| I-270′ | 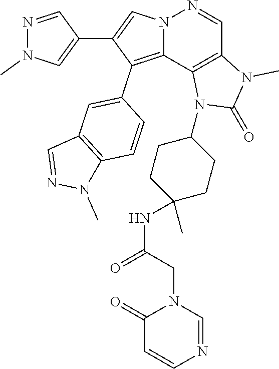 |
| I-271 | 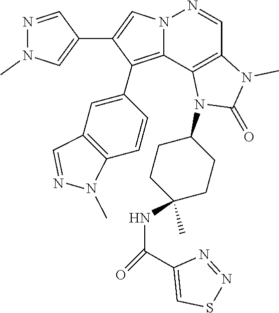 |
| I-271′ | 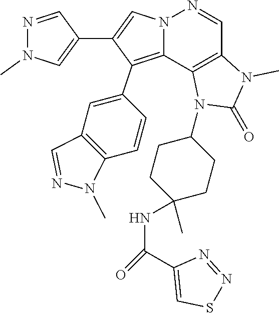 |
| I-272 | 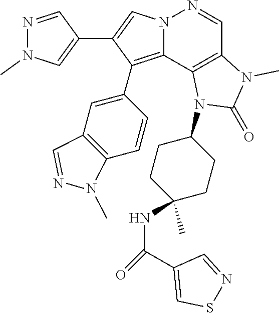 |
| I-272′ | 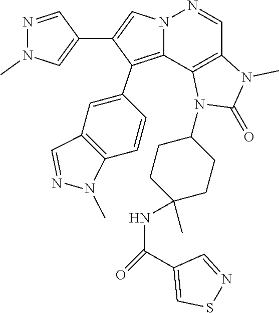 |
| I-273 | 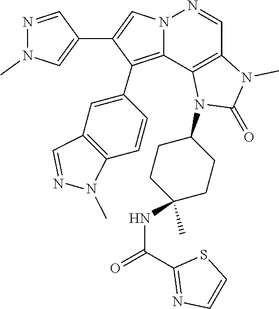 |
| I-273′ | 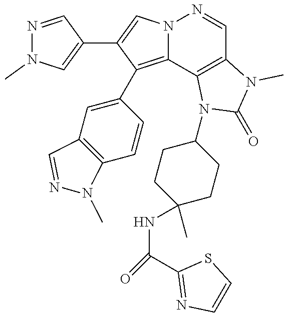 |
| I-274 | 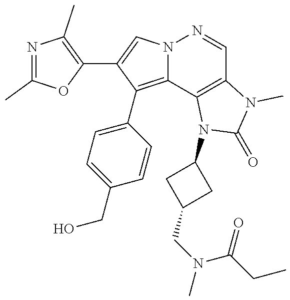 [SMILES: CCC(=O)N(C)C[C@H]1C[C@H](n2c(=O)n(C)c3cnn4cc(-c5oc(C)nc5C)c(-c5ccc(CO)cc5)c4c32)C1] |
| I-274′ | 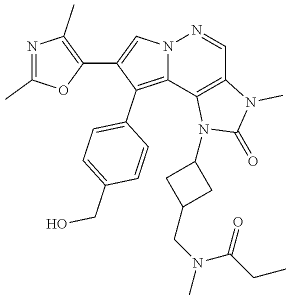 [SMILES: CCC(=O)N(C)CC1CC(n2c(=O)n(C)c3cnn4cc(-c5oc(C)nc5C)c(-c5ccc(CO)cc5)c4c32)C1] |
| I-275 | 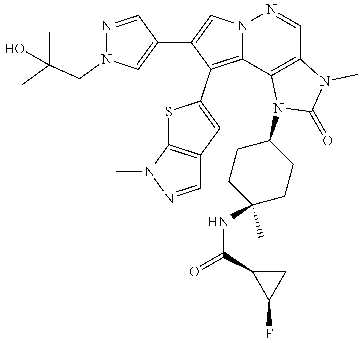 [SMILES: Cn1ncc2cc(-c3c(-c4cnn(CC(C)(C)O)c4)cn4ncc5c(c34)n([C@H]3CC[C@@](C)(NC(=O)[C@H]4C[C@H]4F)CC3)c(=O)n5C)sc21] |
| I-275′ | 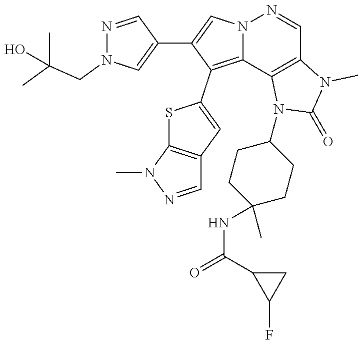 [SMILES: Cn1ncc2cc(-c3c(-c4cnn(CC(C)(C)O)c4)cn4ncc5c(c34)n(C3CCC(C)(NC(=O)C4CC4F)CC3)c(=O)n5C)sc21] |
| I-276 | 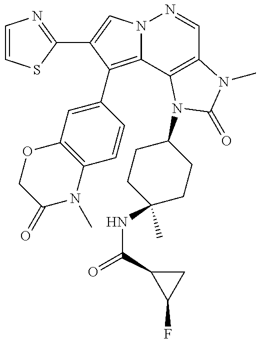 [SMILES: CN1C(=O)COc2cc(-c3c(-c4nccs4)cn4ncc5c(c34)n([C@H]3CC[C@@](C)(NC(=O)[C@H]4C[C@H]4F)CC3)c(=O)n5C)ccc21] |
| I-276′ | 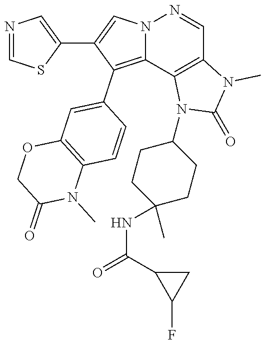 [SMILES: CN1C(=O)COc2cc(-c3c(-c4cncs4)cn4ncc5c(c34)n(C3CCC(C)(NC(=O)C4CC4F)CC3)c(=O)n5C)ccc21] |
| I-277 | 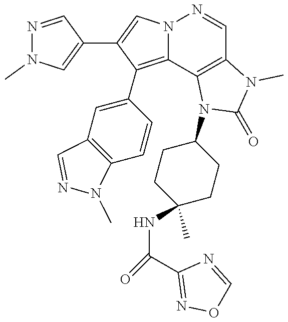 |
| I-277′ | 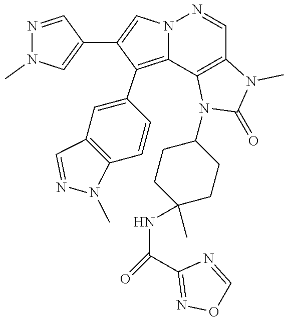 |
| I-278 | 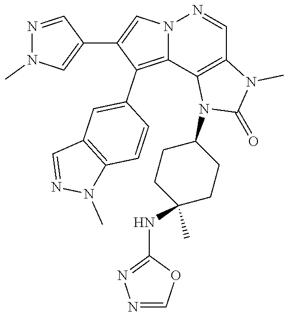 |
| I-278′ | 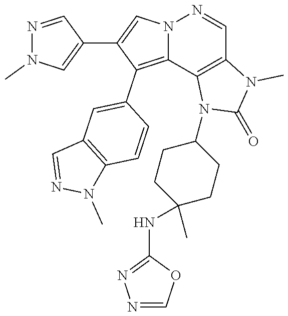 |
| I-279 | 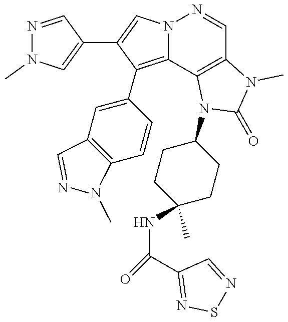 |
| I-279′ | 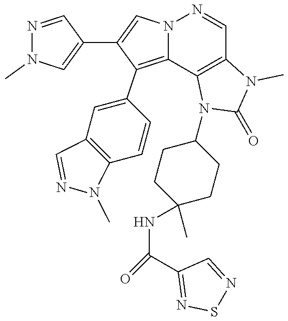 |
| I-280 | 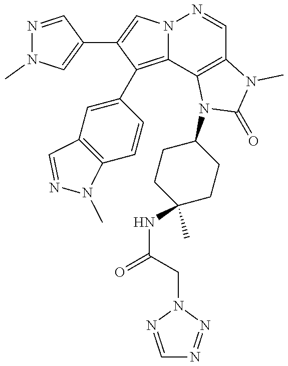 |
| I-280′ | 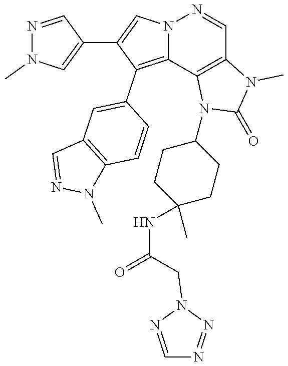 |
| I-281 | 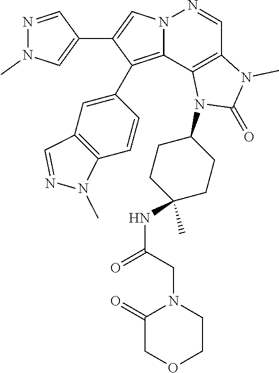 |
| I-281′ | 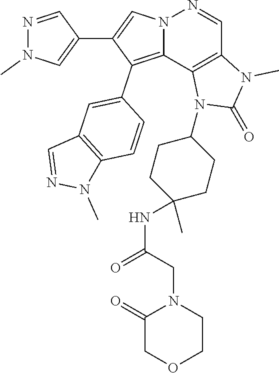 |
| I-282-1 | 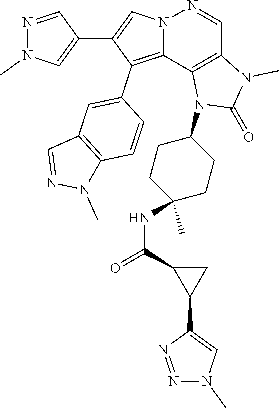 |
| I-282-2 | 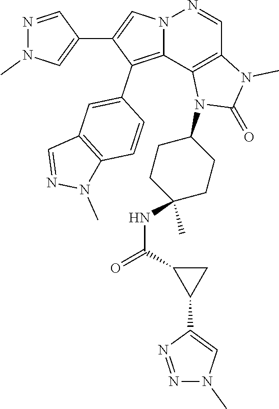 |
| I-282′ | 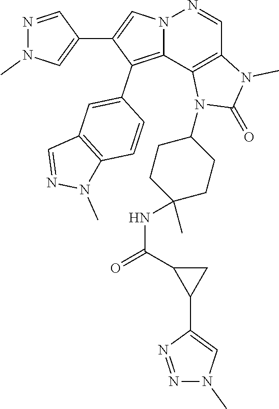 |
| I-283-1 | 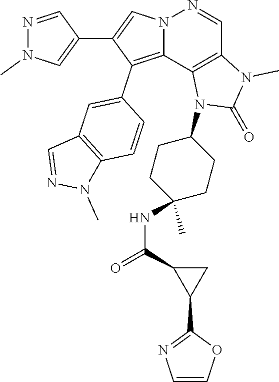 |
| I-283-2 | 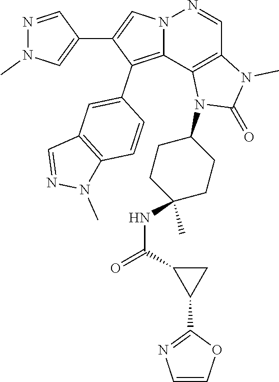 |
| I-283′ | 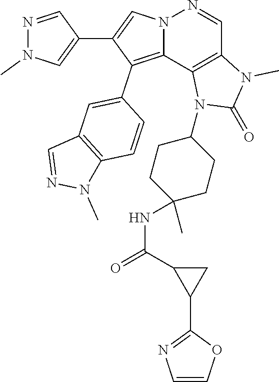 |
| I-284 | 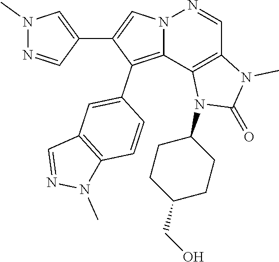 |
| I-284′ | 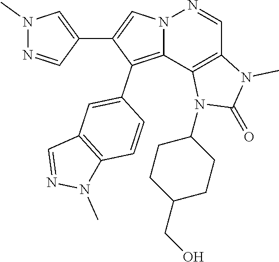 |
| I-285 | 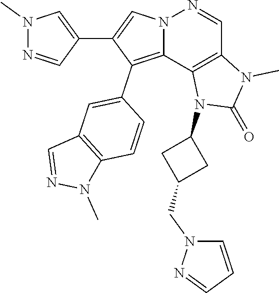 |
| I-285′ | 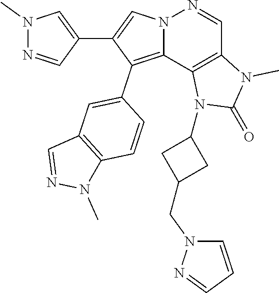 |
| I-286 | 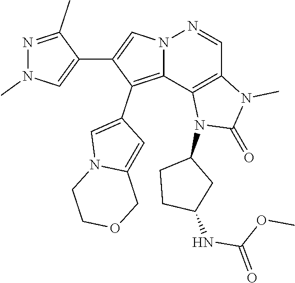 |
| I-286′ | 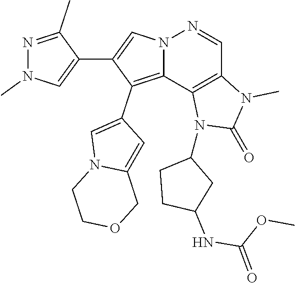 |
| I-287 | 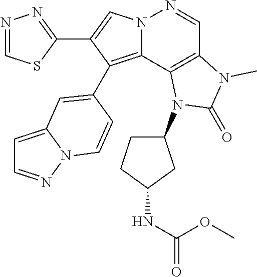 [SMILES: COC(=O)N[C@@H]1CC[C@@H](n2c(=O)n(C)c3cnn4cc(-c5nncs5)c(-c5ccn6nccc6c5)c4c32)C1] |
| I-287′ | 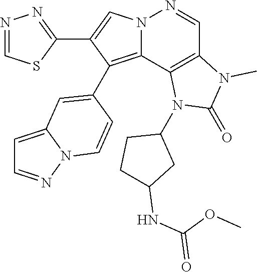 [SMILES: COC(=O)NC1CCC(n2c(=O)n(C)c3cnn4cc(-c5nncs5)c(-c5ccn6nccc6c5)c4c32)C1] |
| I-288 | 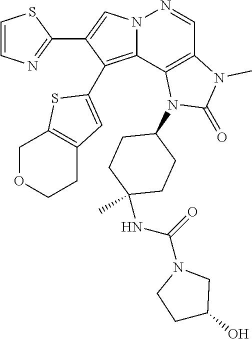 [SMILES: Cn1c(=O)n([C@H]2CC[C@@](C)(NC(=O)N3CC[C@@H](O)C3)CC2)c2c1cnn1cc(-c3nccs3)c(-c3cc4c(s3)COCC4)c21] |
| I-288′ | 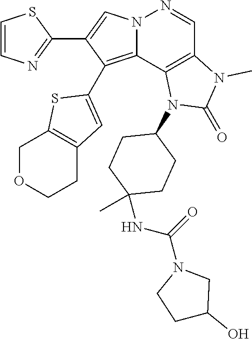 |
| I-289 | 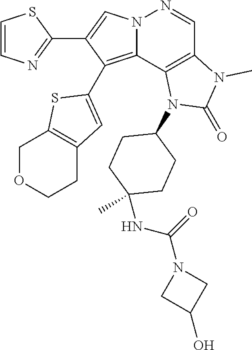 [SMILES: Cn1c(=O)n([C@H]2CC[C@@](C)(NC(=O)N3CC(O)C3)CC2)c2c1cnn1cc(-c3nccs3)c(-c3cc4c(s3)COCC4)c21] |
| I-289′ | 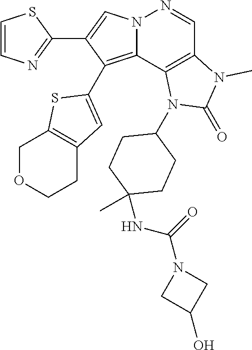 [SMILES: Cn1c(=O)n(C2CCC(C)(NC(=O)N3CC(O)C3)CC2)c2c1cnn1cc(-c3nccs3)c(-c3cc4c(s3)COCC4)c21] |
| I-290 | 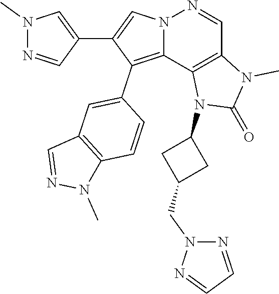 |
| I-290′ | 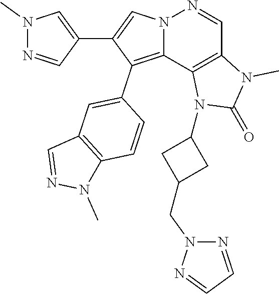 |
| I-291 | 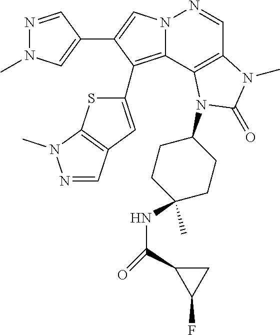 |
| I-291′ | 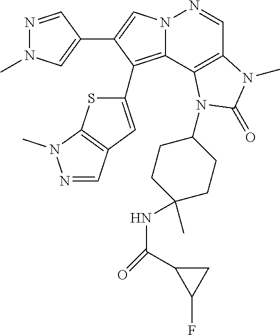 |
| I-292 | 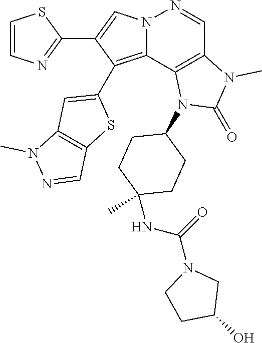 |
| I-292′ | 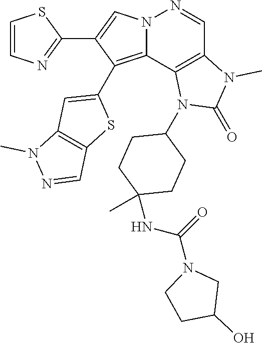 |
| I-293 | 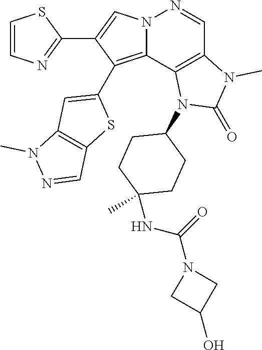 |
| I-293′ | 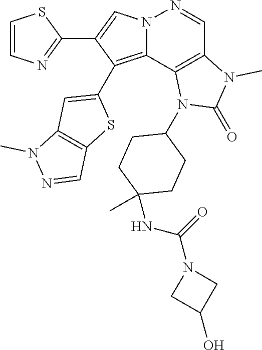 |
| I-294 | 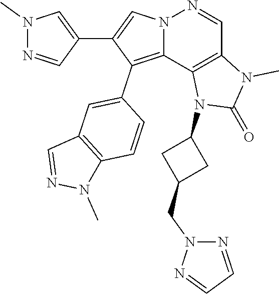 |
| I-294′ | 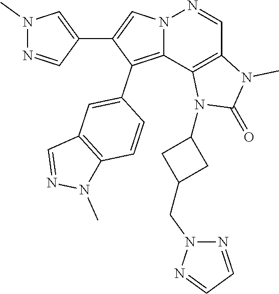 |
| I-295 | 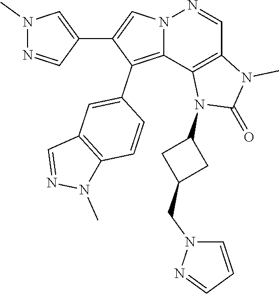 |
| I-295′ | 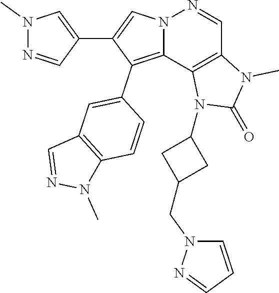 |
| I-296 | 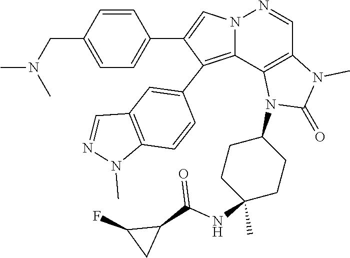 |
| I-296′ | 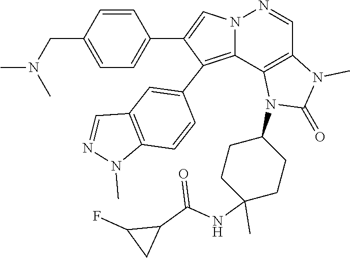 |
| I-297-1 | 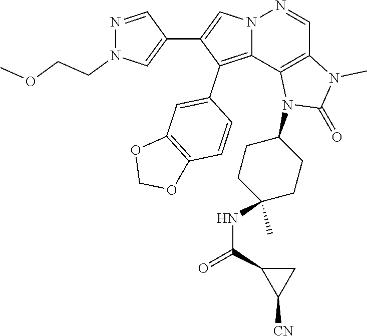 |
| I-297-2 | 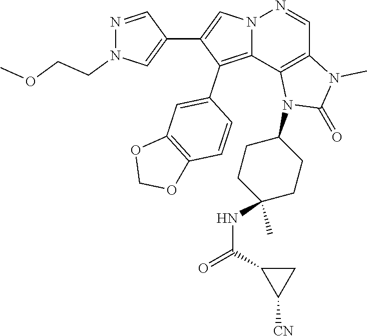 |
| I-297′ | 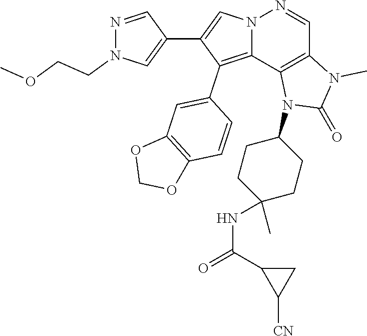 |
| I-298 | 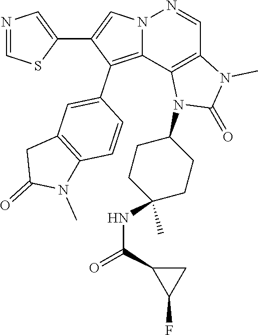 [SMILES: CN1C(=O)Cc2cc(-c3c(-c4cncs4)cn4ncc5c(c34)n([C@H]3CC[C@@](C)(NC(=O)[C@H]4C[C@H]4F)CC3)c(=O)n5C)ccc21] |
| I-298′ | 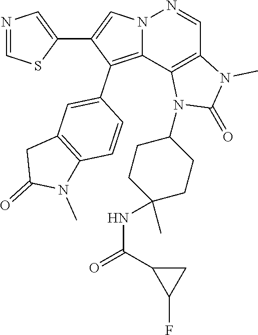 [SMILES: CN1C(=O)Cc2cc(-c3c(-c4cncs4)cn4ncc5c(c34)n(C3CCC(C)(NC(=O)C4CC4F)CC3)c(=O)n5C)ccc21] |
| I-299-1 | 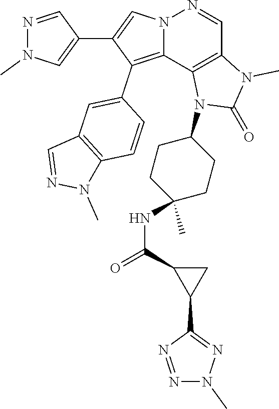 |
| I-299-2 | 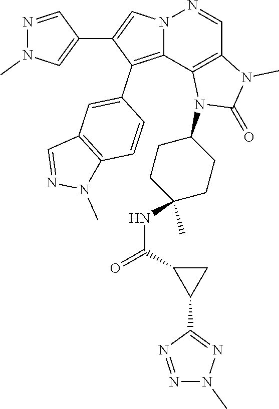 |
| I-299′ | 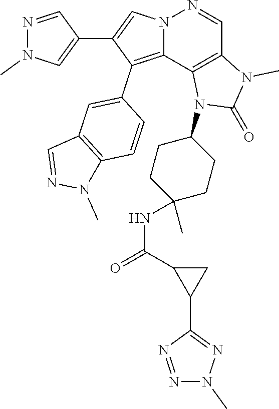 |
| I-300-1 | 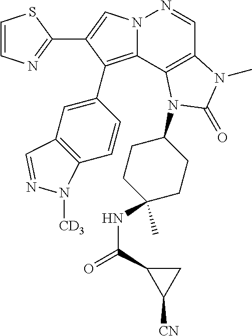 [SMILES: [2H]C([2H])([2H])n1ncc2cc(-c3c(-c4nccs4)cn4ncc5c(c34)n([C@H]3CC[C@@](C)(NC(=O)[C@H]4C[C@H]4C#N)CC3)c(=O)n5C)ccc21] |
| I-300-2 | 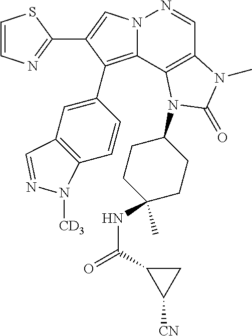 [SMILES: [2H]C([2H])([2H])n1ncc2cc(-c3c(-c4nccs4)cn4ncc5c(c34)n([C@H]3CC[C@@](C)(NC(=O)[C@@H]4C[C@@H]4C#N)CC3)c(=O)n5C)ccc21] |
| I-300′ | 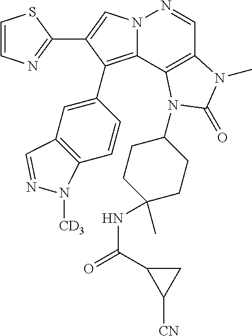 [SMILES: [2H]C([2H])([2H])n1ncc2cc(-c3c(-c4nccs4)cn4ncc5c(c34)n(C3CCC(C)(NC(=O)C4CC4C#N)CC3)c(=O)n5C)ccc21] |
| I-301-1 | 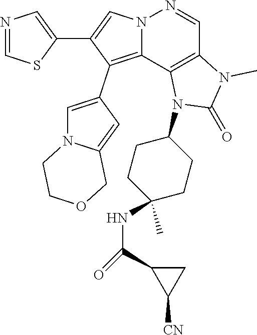 [SMILES: Cn1c(=O)n([C@H]2CC[C@@](C)(NC(=O)[C@H]3C[C@H]3C#N)CC2)c2c1cnn1cc(-c3cncs3)c(-c3cc4n(c3)CCOC4)c21] |
| I-301-2 | 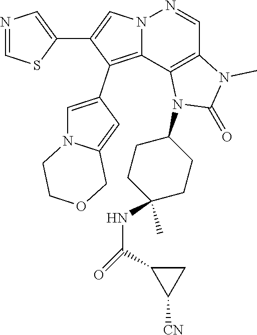 [SMILES: Cn1c(=O)n([C@H]2CC[C@@](C)(NC(=O)[C@@H]3C[C@@H]3C#N)CC2)c2c1cnn1cc(-c3cncs3)c(-c3cc4n(c3)CCOC4)c21] |
| I-301′ | 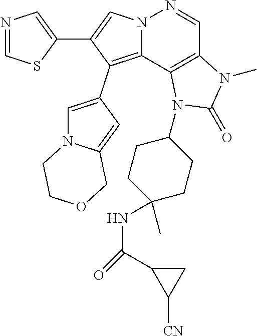 [SMILES: Cn1c(=O)n(C2CCC(C)(NC(=O)C3CC3C#N)CC2)c2c1cnn1cc(-c3cncs3)c(-c3cc4n(c3)CCOC4)c21] |
| I-302 | 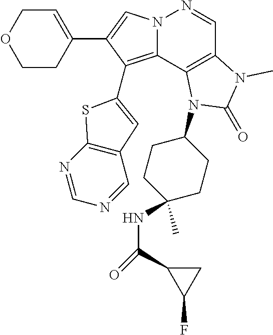 [SMILES: Cn1c(=O)n([C@H]2CC[C@@](C)(NC(=O)[C@H]3C[C@H]3F)CC2)c2c1cnn1cc(C3=CCOCC3)c(-c3cc4cncnc4s3)c21] |
| I-302′ | 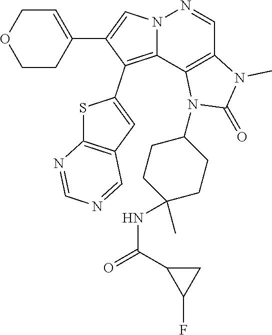 [SMILES: Cn1c(=O)n(C2CCC(C)(NC(=O)C3CC3F)CC2)c2c1cnn1cc(C3=CCOCC3)c(-c3cc4cncnc4s3)c21] |
| I-303-1 | 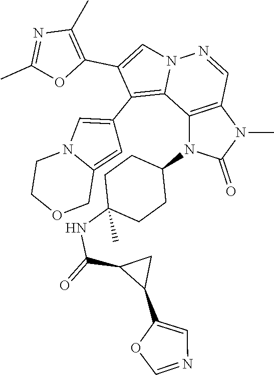 |
| I-303-2 | 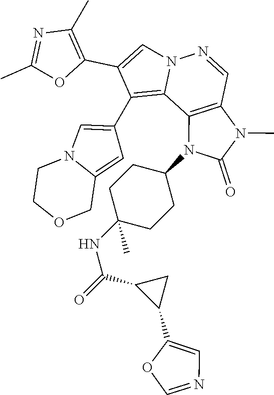 |
| I-303′ | 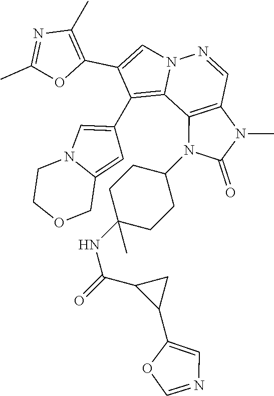 |
| I-304-1 | 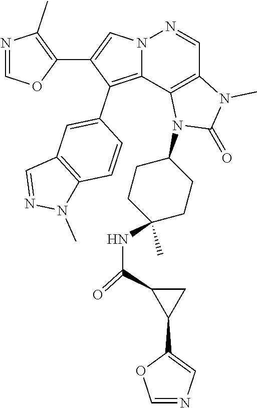 [SMILES: Cc1ncoc1-c1cn2ncc3c(c2c1-c1ccc2c(cnn2C)c1)n([C@H]1CC[C@@](C)(NC(=O)[C@H]2C[C@H]2c2cnco2)CC1)c(=O)n3C] |
| I-304-2 | 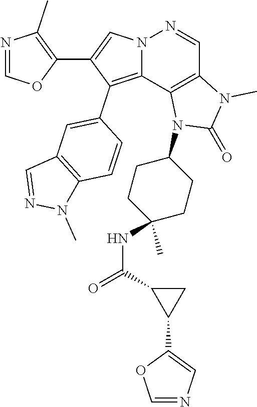 [SMILES: Cc1ncoc1-c1cn2ncc3c(c2c1-c1ccc2c(cnn2C)c1)n([C@H]1CC[C@@](C)(NC(=O)[C@@H]2C[C@@H]2c2cnco2)CC1)c(=O)n3C] |
| I-304′ | 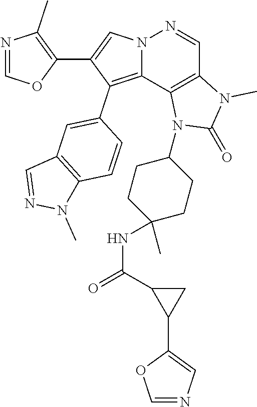 [SMILES: Cc1ncoc1-c1cn2ncc3c(c2c1-c1ccc2c(cnn2C)c1)n(C1CCC(C)(NC(=O)C2CC2c2cnco2)CC1)c(=O)n3C] |
| I-305 | 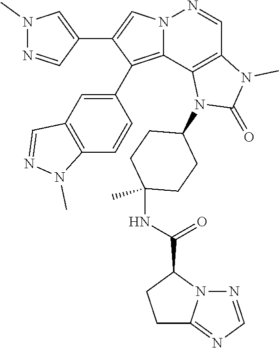 |
| I-305′ | 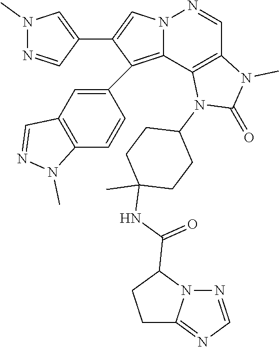 |
| I-306 | 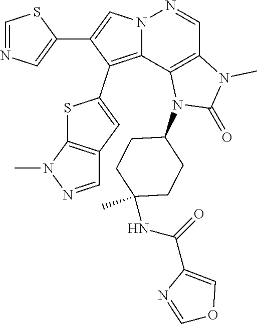 [SMILES: Cn1ncc2cc(-c3c(-c4cncs4)cn4ncc5c(c34)n([C@H]3CC[C@@](C)(NC(=O)c4cocn4)CC3)c(=O)n5C)sc21] |
| I-306′ | 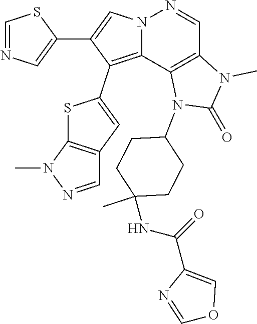 [SMILES: Cn1ncc2cc(-c3c(-c4cncs4)cn4ncc5c(c34)n(C3CCC(C)(NC(=O)c4cocn4)CC3)c(=O)n5C)sc21] |
| I-307 | 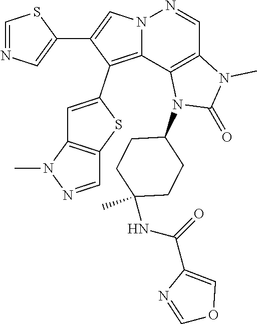 [SMILES: Cn1ncc2sc(-c3c(-c4cncs4)cn4ncc5c(c34)n([C@H]3CC[C@@](C)(NC(=O)c4cocn4)CC3)c(=O)n5C)cc21] |
| I-307′ | 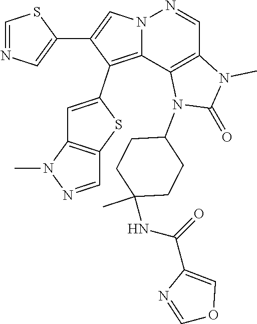 [SMILES: Cn1ncc2sc(-c3c(-c4cncs4)cn4ncc5c(c34)n(C3CCC(C)(NC(=O)c4cocn4)CC3)c(=O)n5C)cc21] |
| I-308 | 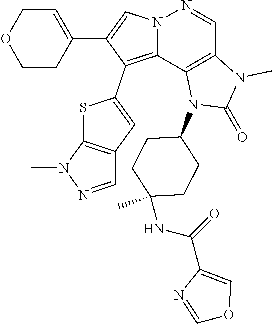 [SMILES: Cn1ncc2cc(-c3c(C4=CCOCC4)cn4ncc5c(c34)n([C@H]3CC[C@@](C)(NC(=O)c4cocn4)CC3)c(=O)n5C)sc21] |
| I-308′ | 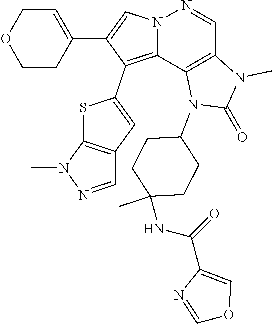 [SMILES: Cn1ncc2cc(-c3c(C4=CCOCC4)cn4ncc5c(c34)n(C3CCC(C)(NC(=O)c4cocn4)CC3)c(=O)n5C)sc21] |
| I-309 | 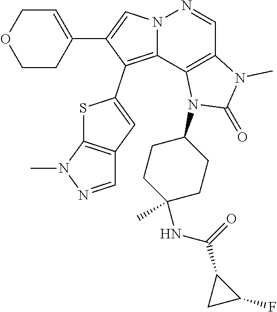 [SMILES: Cn1ncc2cc(-c3c(C4=CCOCC4)cn4ncc5c(c34)n([C@H]3CC[C@@](C)(NC(=O)[C@H]4C[C@H]4F)CC3)c(=O)n5C)sc21] |
| I-309′ | 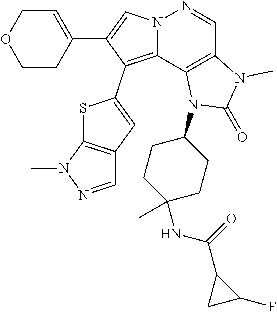 |
| I-310-1 | 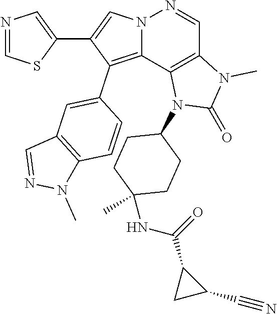 [SMILES: Cn1ncc2cc(-c3c(-c4cncs4)cn4ncc5c(c34)n([C@H]3CC[C@@](C)(NC(=O)[C@H]4C[C@H]4C#N)CC3)c(=O)n5C)ccc21] |
| I-310-2 | 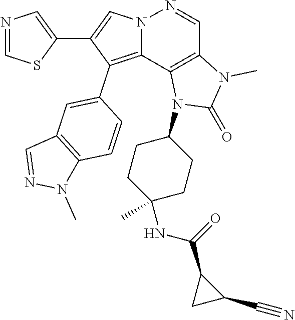 [SMILES: Cn1ncc2cc(-c3c(-c4cncs4)cn4ncc5c(c34)n([C@H]3CC[C@@](C)(NC(=O)[C@@H]4C[C@@H]4C#N)CC3)c(=O)n5C)ccc21] |
| I-310′ | 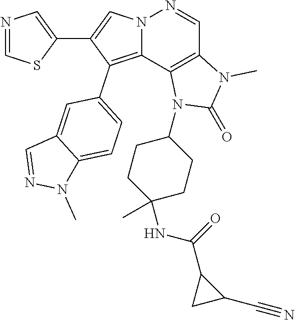 [SMILES: Cn1ncc2cc(-c3c(-c4cncs4)cn4ncc5c(c34)n(C3CCC(C)(NC(=O)C4CC4C#N)CC3)c(=O)n5C)ccc21] |
| I-311 | 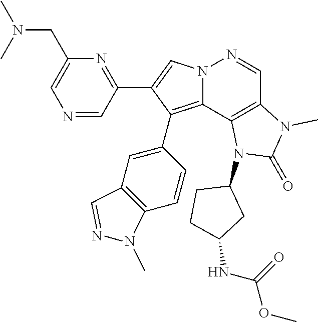 |
| I-311′ | 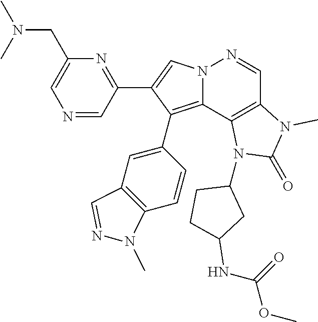 |
| I-312 | 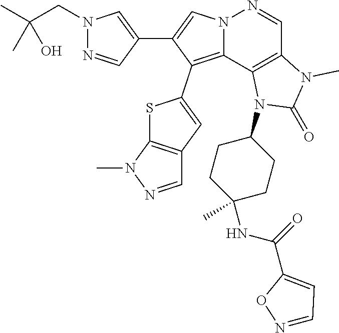 [SMILES: Cn1ncc2cc(-c3c(-c4cnn(CC(C)(C)O)c4)cn4ncc5c(c34)n([C@H]3CC[C@@](C)(NC(=O)c4ccno4)CC3)c(=O)n5C)sc21] |
| I-312′ | 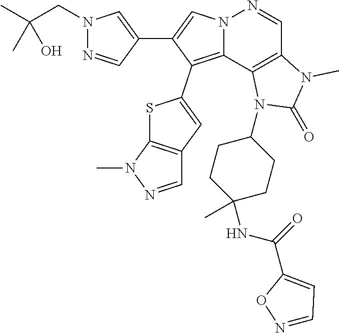 [SMILES: Cn1ncc2cc(-c3c(-c4cnn(CC(C)(C)O)c4)cn4ncc5c(c34)n(C3CCC(C)(NC(=O)c4ccno4)CC3)c(=O)n5C)sc21] |
| I-313 | 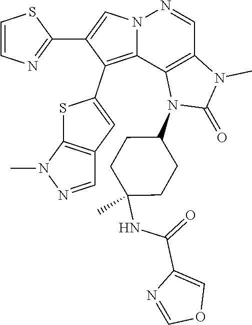 |
| I-313′ | 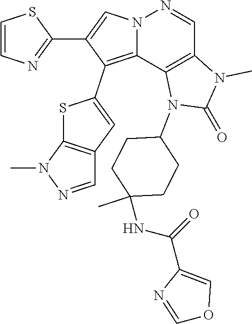 [SMILES: Cn1ncc2cc(-c3c(-c4nccs4)cn4ncc5c(c34)n(C3CCC(C)(NC(=O)c4cocn4)CC3)c(=O)n5C)sc21] |
| I-314 | 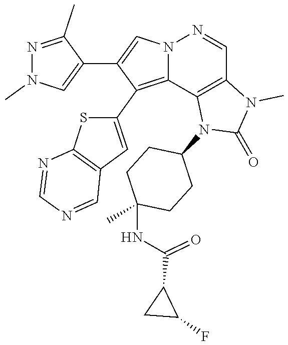 |
| I-314′ | 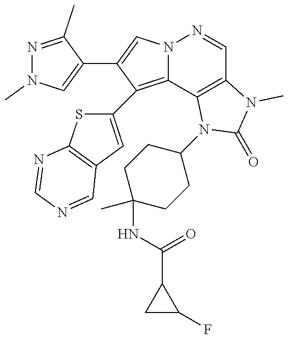 |
| I-315 | 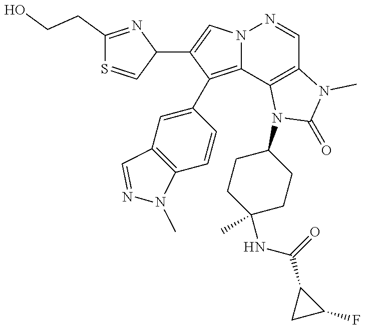 |
| I-315′ | 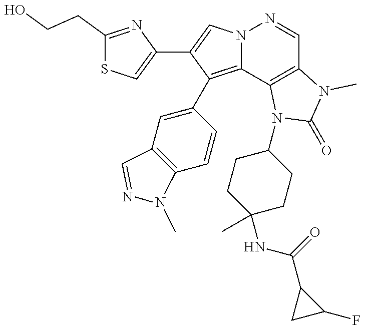 [SMILES: Cn1ncc2cc(-c3c(-c4csc(CCO)n4)cn4ncc5c(c34)n(C3CCC(C)(NC(=O)C4CC4F)CC3)c(=O)n5C)ccc21] |
| I-316 | 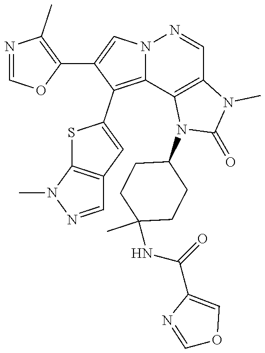 |
| I-316′ | 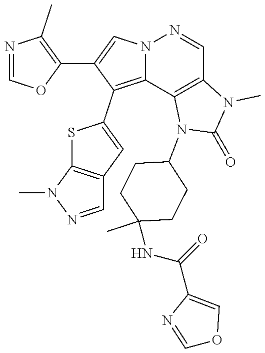 [SMILES: Cc1ncoc1-c1cn2ncc3c(c2c1-c1cc2cnn(C)c2s1)n(C1CCC(C)(NC(=O)c2cocn2)CC1)c(=O)n3C] |
| I-317 | 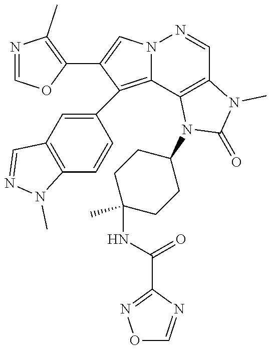 [SMILES: Cc1ncoc1-c1cn2ncc3c(c2c1-c1ccc2c(cnn2C)c1)n([C@H]1CC[C@@](C)(NC(=O)c2ncon2)CC1)c(=O)n3C] |
| I-317′ | 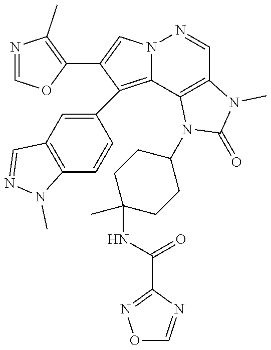 [SMILES: Cc1ncoc1-c1cn2ncc3c(c2c1-c1ccc2c(cnn2C)c1)n(C1CCC(C)(NC(=O)c2ncon2)CC1)c(=O)n3C] |
| I-318 | 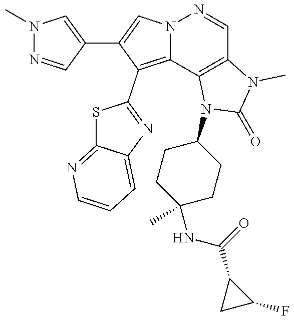 [SMILES: Cn1cc(-c2cn3ncc4c(c3c2-c2nc3cccnc3s2)n([C@H]2CC[C@@](C)(NC(=O)[C@H]3C[C@H]3F)CC2)c(=O)n4C)cn1] |
| I-318′ | 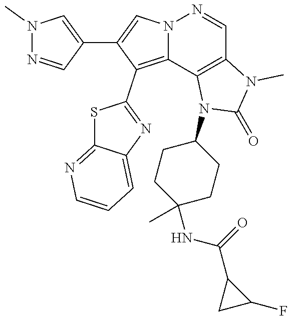 |
| I-319 | 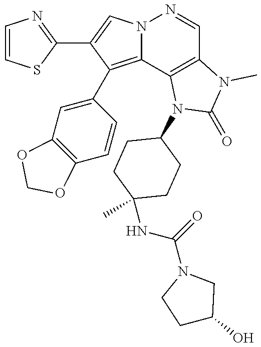 [SMILES: Cn1c(=O)n([C@H]2CC[C@@](C)(NC(=O)N3CC[C@@H](O)C3)CC2)c2c1cnn1cc(-c3nccs3)c(-c3ccc4c(c3)OCO4)c21] |
| I-319′ | 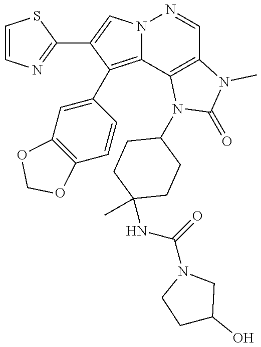 [SMILES: Cn1c(=O)n(C2CCC(C)(NC(=O)N3CCC(O)C3)CC2)c2c1cnn1cc(-c3nccs3)c(-c3ccc4c(c3)OCO4)c21] |
| I-320 | 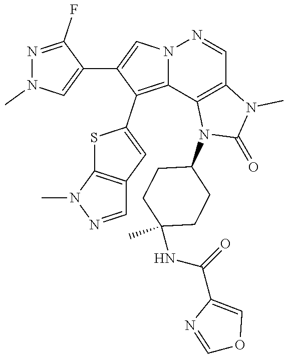 |
| I-320′ | 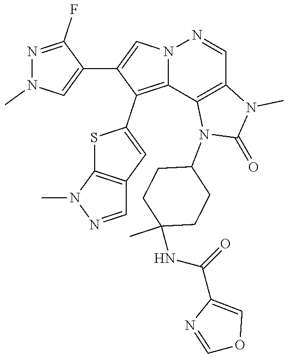 |
| I-321-1 | 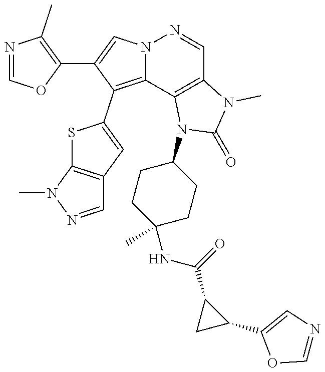 [SMILES: Cc1ncoc1-c1cn2ncc3c(c2c1-c1cc2cnn(C)c2s1)n([C@H]1CC[C@@](C)(NC(=O)[C@H]2C[C@H]2c2cnco2)CC1)c(=O)n3C] |
| I-321-2 | 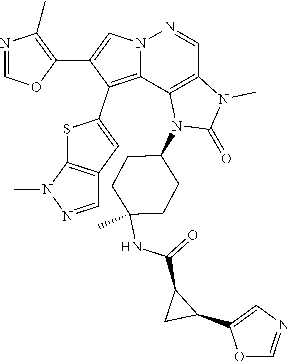 [SMILES: Cc1ncoc1-c1cn2ncc3c(c2c1-c1cc2cnn(C)c2s1)n([C@H]1CC[C@@](C)(NC(=O)[C@@H]2C[C@@H]2c2cnco2)CC1)c(=O)n3C] |
| I-321′ | 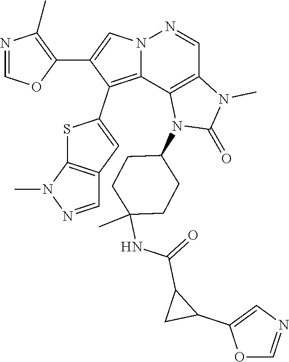 |
| I-322 | 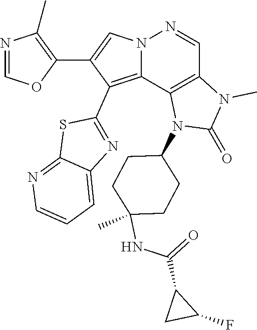 [SMILES: Cc1ncoc1-c1cn2ncc3c(c2c1-c1nc2cccnc2s1)n([C@H]1CC[C@@](C)(NC(=O)[C@H]2C[C@H]2F)CC1)c(=O)n3C] |
| I-322′ | 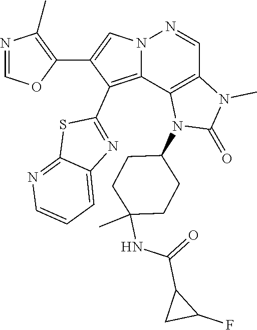 |
| I-323 | 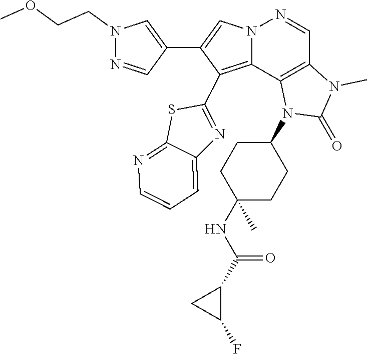 |
| I-323′ | 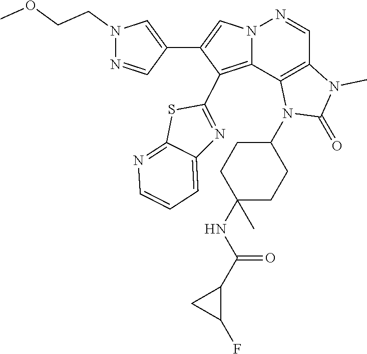 |
| I-324 | 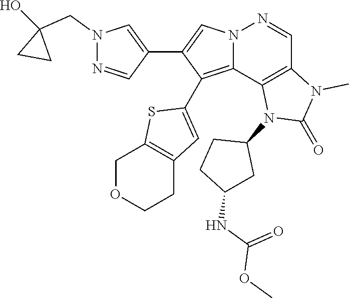 [SMILES: COC(=O)N[C@@H]1CC[C@@H](n2c(=O)n(C)c3cnn4cc(-c5cnn(CC6(O)CC6)c5)c(-c5cc6c(s5)COCC6)c4c32)C1] |
| I-324′ | 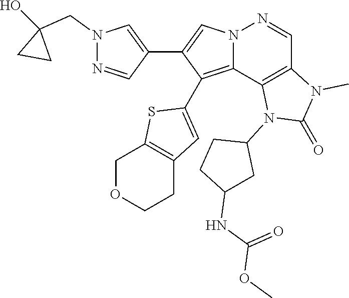 [SMILES: COC(=O)NC1CCC(n2c(=O)n(C)c3cnn4cc(-c5cnn(CC6(O)CC6)c5)c(-c5cc6c(s5)COCC6)c4c32)C1] |
| I-325 | 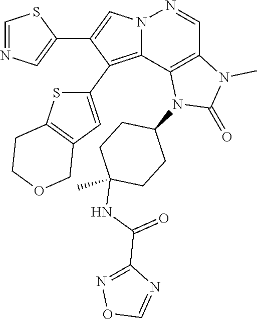 [SMILES: Cn1c(=O)n([C@H]2CC[C@@](C)(NC(=O)c3ncon3)CC2)c2c1cnn1cc(-c3cncs3)c(-c3cc4c(s3)CCOC4)c21] |
| I-325′ | 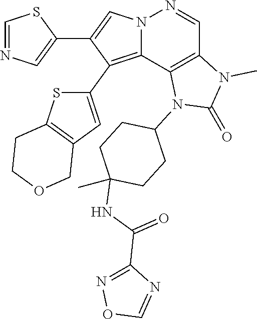 [SMILES: Cn1c(=O)n(C2CCC(C)(NC(=O)c3ncon3)CC2)c2c1cnn1cc(-c3cncs3)c(-c3cc4c(s3)CCOC4)c21] |
| I-326-1 | 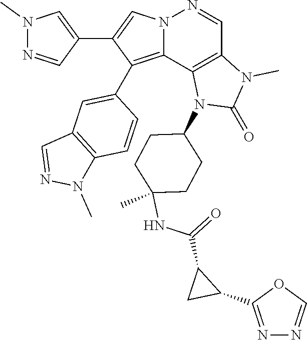 [SMILES: Cn1cc(-c2cn3ncc4c(c3c2-c2ccc3c(cnn3C)c2)n([C@H]2CC[C@@](C)(NC(=O)[C@H]3C[C@H]3c3nnco3)CC2)c(=O)n4C)cn1] |
| I-326-2 | 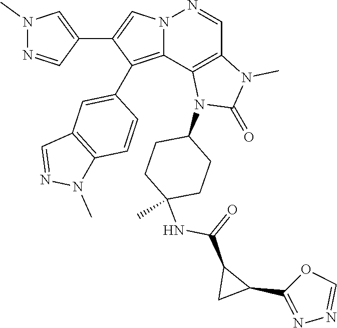 [SMILES: Cn1cc(-c2cn3ncc4c(c3c2-c2ccc3c(cnn3C)c2)n([C@H]2CC[C@@](C)(NC(=O)[C@@H]3C[C@@H]3c3nnco3)CC2)c(=O)n4C)cn1] |
| I-326′ | 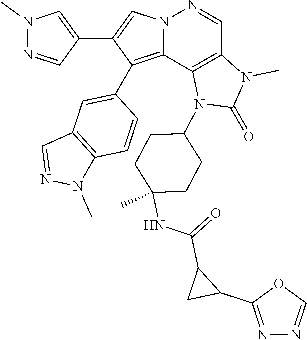 |
| I-327 | 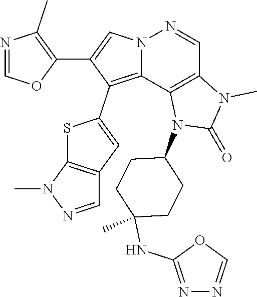 [SMILES: Cc1ncoc1-c1cn2ncc3c(c2c1-c1cc2cnn(C)c2s1)n([C@H]1CC[C@@](C)(Nc2nnco2)CC1)c(=O)n3C] |
| I-327′ | 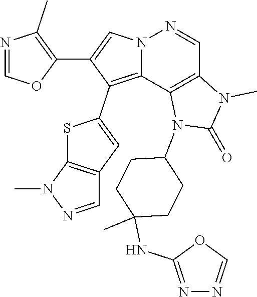 [SMILES: Cc1ncoc1-c1cn2ncc3c(c2c1-c1cc2cnn(C)c2s1)n(C1CCC(C)(Nc2nnco2)CC1)c(=O)n3C] |
| I-328 | 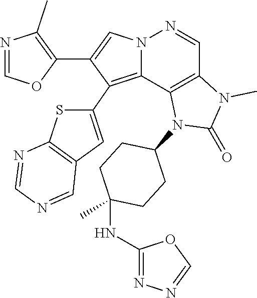 [SMILES: Cc1ncoc1-c1cn2ncc3c(c2c1-c1cc2cncnc2s1)n([C@H]1CC[C@@](C)(Nc2nnco2)CC1)c(=O)n3C] |
| I-328′ | 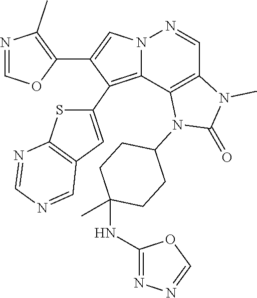 [SMILES: Cc1ncoc1-c1cn2ncc3c(c2c1-c1cc2cncnc2s1)n(C1CCC(C)(Nc2nnco2)CC1)c(=O)n3C] |
| I-329-1 | 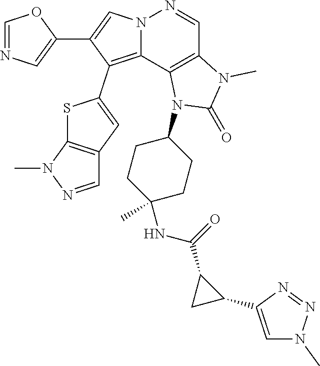 [SMILES: Cn1cc([C@@H]2C[C@@H]2C(=O)N[C@]2(C)CC[C@H](n3c(=O)n(C)c4cnn5cc(-c6cnco6)c(-c6cc7cnn(C)c7s6)c5c43)CC2)nn1] |
| I-329-2 | 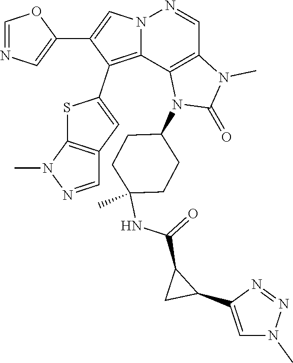 [SMILES: Cn1cc([C@H]2C[C@H]2C(=O)N[C@]2(C)CC[C@H](n3c(=O)n(C)c4cnn5cc(-c6cnco6)c(-c6cc7cnn(C)c7s6)c5c43)CC2)nn1] |
| I-329′ | 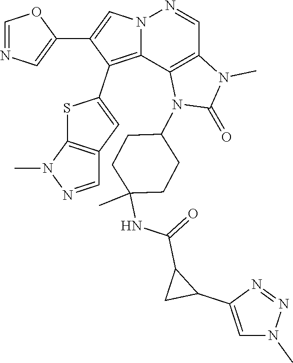 [SMILES: Cn1cc(C2CC2C(=O)NC2(C)CCC(n3c(=O)n(C)c4cnn5cc(-c6cnco6)c(-c6cc7cnn(C)c7s6)c5c43)CC2)nn1] |
| I-330-1 | 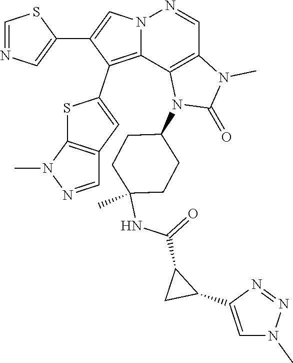 [SMILES: Cn1cc([C@@H]2C[C@@H]2C(=O)N[C@]2(C)CC[C@H](n3c(=O)n(C)c4cnn5cc(-c6cncs6)c(-c6cc7cnn(C)c7s6)c5c43)CC2)nn1] |
| I-330-2 | 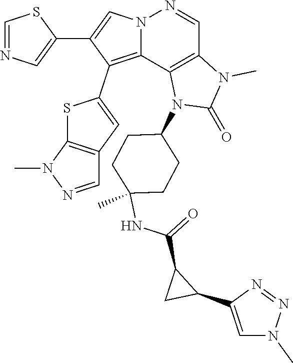 [SMILES: Cn1cc([C@H]2C[C@H]2C(=O)N[C@]2(C)CC[C@H](n3c(=O)n(C)c4cnn5cc(-c6cncs6)c(-c6cc7cnn(C)c7s6)c5c43)CC2)nn1] |
| I-330′ | 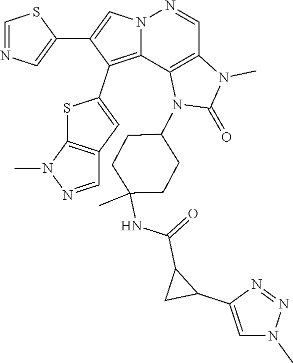 [SMILES: Cn1cc(C2CC2C(=O)NC2(C)CCC(n3c(=O)n(C)c4cnn5cc(-c6cncs6)c(-c6cc7cnn(C)c7s6)c5c43)CC2)nn1] |
| I-331 | 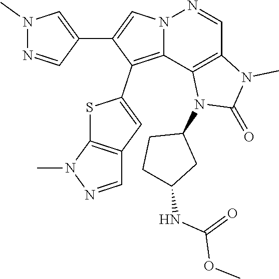 |
| I-331′ | 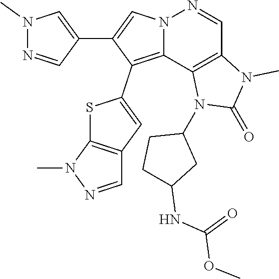 |
| I-332 | 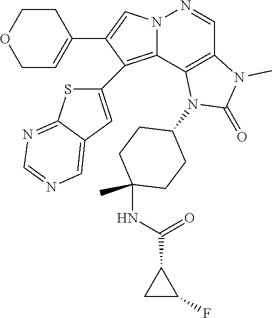 [SMILES: Cn1c(=O)n([C@H]2CC[C@@](C)(NC(=O)[C@H]3C[C@H]3F)CC2)c2c1cnn1cc(C3=CCOCC3)c(-c3cc4cncnc4s3)c21] |
| I-332′ | 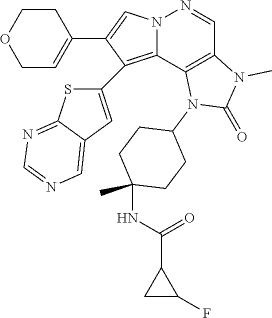 |
| I-333 | 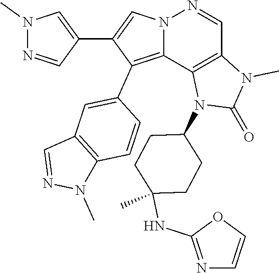 |
| I-333′ | 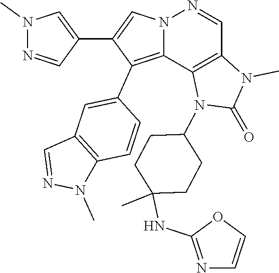 |
| I-334 | 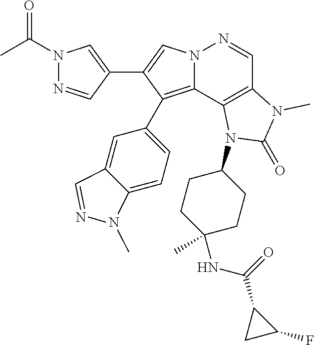 [SMILES: CC(=O)n1cc(-c2cn3ncc4c(c3c2-c2ccc3c(cnn3C)c2)n([C@H]2CC[C@@](C)(NC(=O)[C@H]3C[C@H]3F)CC2)c(=O)n4C)cn1] |
| I-334′ | 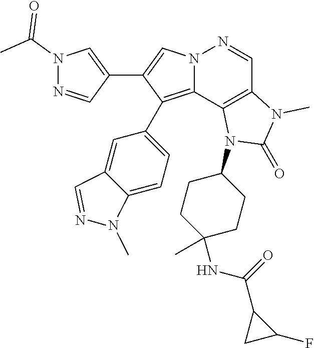 |
| I-335 | 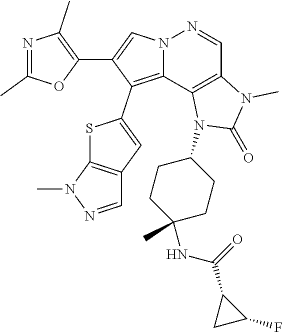 [SMILES: Cc1nc(C)c(-c2cn3ncc4c(c3c2-c2cc3cnn(C)c3s2)n([C@H]2CC[C@@](C)(NC(=O)[C@H]3C[C@H]3F)CC2)c(=O)n4C)o1] |
| I-335′ | 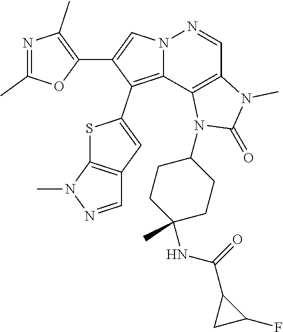 |
| I-336 | 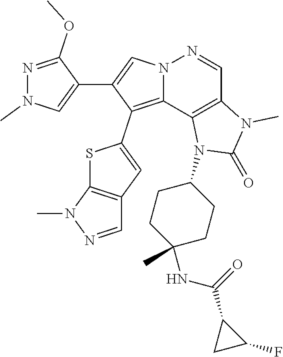 [SMILES: COc1nn(C)cc1-c1cn2ncc3c(c2c1-c1cc2cnn(C)c2s1)n([C@H]1CC[C@@](C)(NC(=O)[C@H]2C[C@H]2F)CC1)c(=O)n3C] |
| I-336′ | 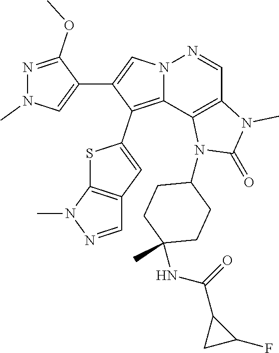 |
| I-337 | 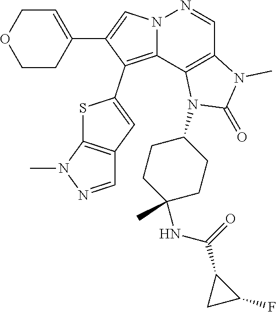 [SMILES: Cn1ncc2cc(-c3c(C4=CCOCC4)cn4ncc5c(c34)n([C@H]3CC[C@@](C)(NC(=O)[C@H]4C[C@H]4F)CC3)c(=O)n5C)sc21] |
| I-337′ | 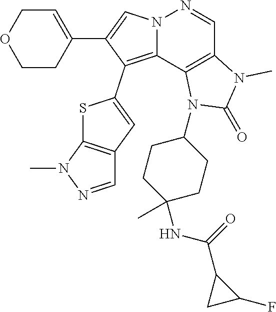 [SMILES: Cn1ncc2cc(-c3c(C4=CCOCC4)cn4ncc5c(c34)n(C3CCC(C)(NC(=O)C4CC4F)CC3)c(=O)n5C)sc21] |
| I-338 | 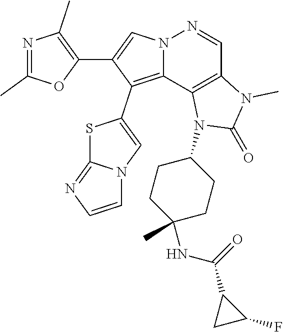 |
| I-338′ | 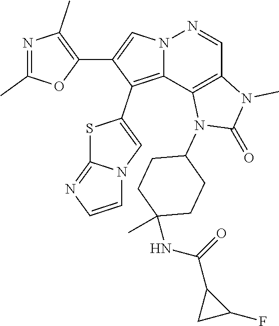 |
| I-339 | 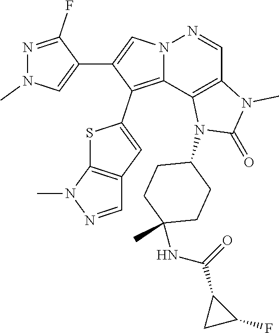 |
| I-339′ | 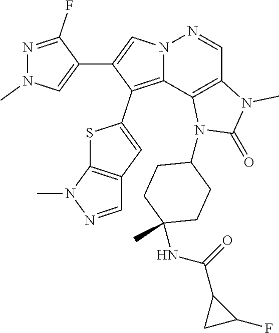 |
| I-340 | 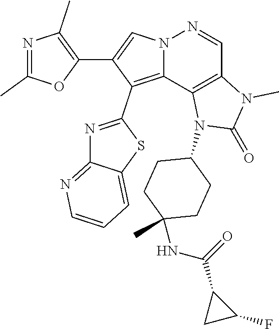 |
| I-340′ | 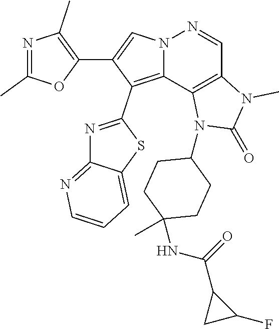 |
| I-341-1 | 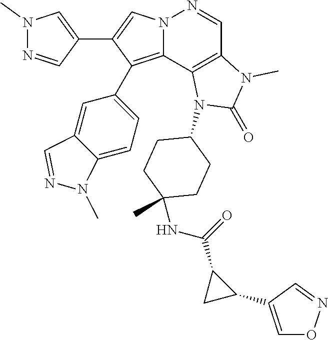 [SMILES: Cn1cc(-c2cn3ncc4c(c3c2-c2ccc3c(cnn3C)c2)n([C@H]2CC[C@@](C)(NC(=O)[C@H]3C[C@H]3c3cnoc3)CC2)c(=O)n4C)cn1] |
| I-341-2 | 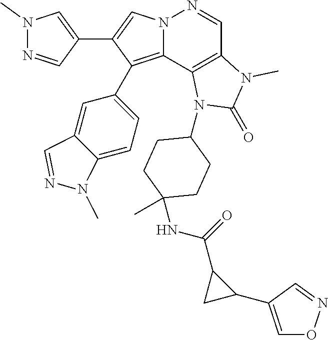 [SMILES: Cn1cc(-c2cn3ncc4c(c3c2-c2ccc3c(cnn3C)c2)n(C2CCC(C)(NC(=O)C3CC3c3cnoc3)CC2)c(=O)n4C)cn1] |
| I-341′ | 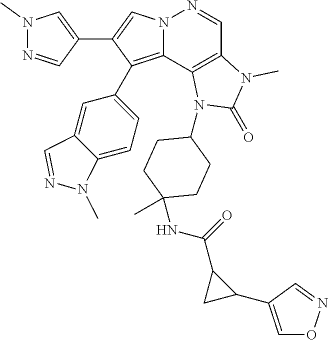 [SMILES: Cn1cc(-c2cn3ncc4c(c3c2-c2ccc3c(cnn3C)c2)n(C2CCC(C)(NC(=O)C3CC3c3cnoc3)CC2)c(=O)n4C)cn1] |
| I-342-1 | 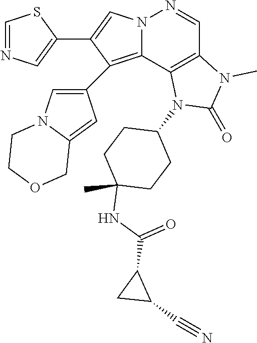 [SMILES: Cn1c(=O)n([C@H]2CC[C@@](C)(NC(=O)[C@H]3C[C@H]3C#N)CC2)c2c1cnn1cc(-c3cncs3)c(-c3cc4n(c3)CCOC4)c21] |
| I-342-2 | 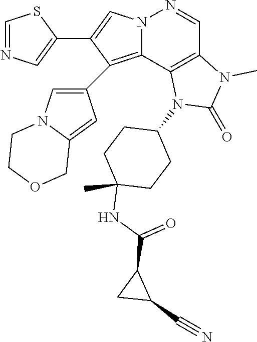 [SMILES: Cn1c(=O)n([C@H]2CC[C@@](C)(NC(=O)[C@@H]3C[C@@H]3C#N)CC2)c2c1cnn1cc(-c3cncs3)c(-c3cc4n(c3)CCOC4)c21] |
| I-342′ | 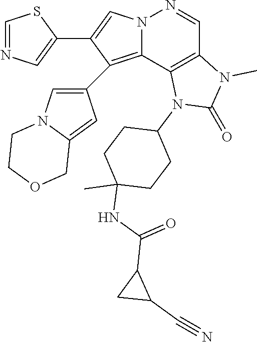 [SMILES: Cn1c(=O)n(C2CCC(C)(NC(=O)C3CC3C#N)CC2)c2c1cnn1cc(-c3cncs3)c(-c3cc4n(c3)CCOC4)c21] |
| I-343-1 | 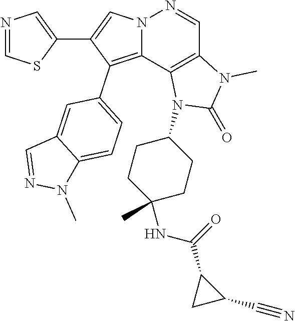 [SMILES: Cn1ncc2cc(-c3c(-c4cncs4)cn4ncc5c(c34)n([C@H]3CC[C@@](C)(NC(=O)[C@H]4C[C@H]4C#N)CC3)c(=O)n5C)ccc21] |
| I-343-2 | 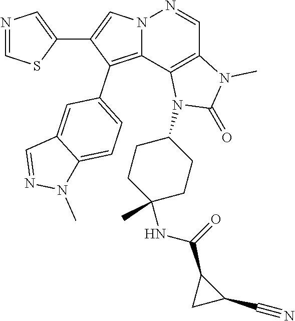 [SMILES: Cn1ncc2cc(-c3c(-c4cncs4)cn4ncc5c(c34)n([C@H]3CC[C@@](C)(NC(=O)[C@@H]4C[C@@H]4C#N)CC3)c(=O)n5C)ccc21] |
| I-343′ | 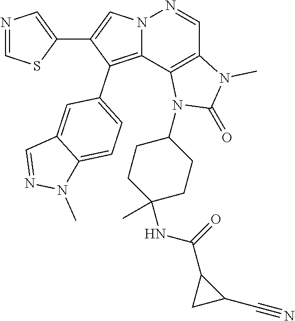 [SMILES: Cn1ncc2cc(-c3c(-c4cncs4)cn4ncc5c(c34)n(C3CCC(C)(NC(=O)C4CC4C#N)CC3)c(=O)n5C)ccc21] |
| I-344 | 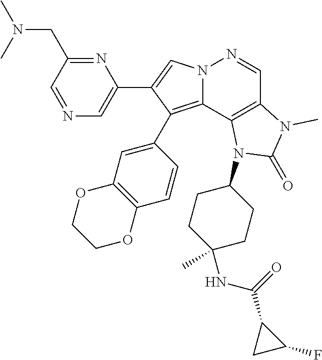 [SMILES: CN(C)Cc1cncc(-c2cn3ncc4c(c3c2-c2ccc3c(c2)OCCO3)n([C@H]2CC[C@@](C)(NC(=O)[C@H]3C[C@H]3F)CC2)c(=O)n4C)n1] |
| I-344′ | 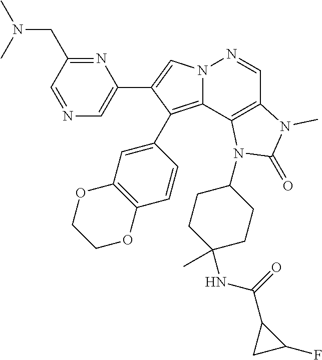 [SMILES: CN(C)Cc1cncc(-c2cn3ncc4c(c3c2-c2ccc3c(c2)OCCO3)n(C2CCC(C)(NC(=O)C3CC3F)CC2)c(=O)n4C)n1] |
| I-345 | 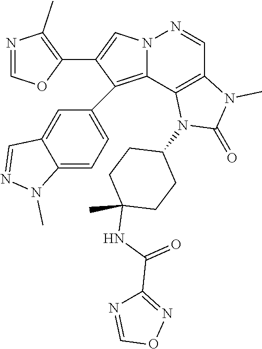 [SMILES: Cc1ncoc1-c1cn2ncc3c(c2c1-c1ccc2c(cnn2C)c1)n([C@H]1CC[C@@](C)(NC(=O)c2ncon2)CC1)c(=O)n3C] |
| I-345′ | 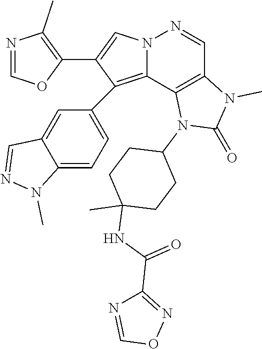 [SMILES: Cc1ncoc1-c1cn2ncc3c(c2c1-c1ccc2c(cnn2C)c1)n(C1CCC(C)(NC(=O)c2ncon2)CC1)c(=O)n3C] |
| I-346 | 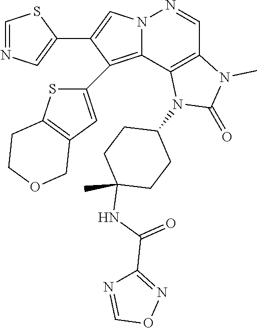 [SMILES: Cn1c(=O)n([C@H]2CC[C@@](C)(NC(=O)c3ncon3)CC2)c2c1cnn1cc(-c3cncs3)c(-c3cc4c(s3)CCOC4)c21] |
| I-346′ | 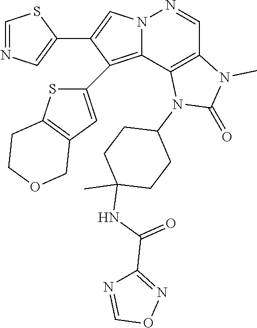 [SMILES: Cn1c(=O)n(C2CCC(C)(NC(=O)c3ncon3)CC2)c2c1cnn1cc(-c3cncs3)c(-c3cc4c(s3)CCOC4)c21] |
| I-347-1 | 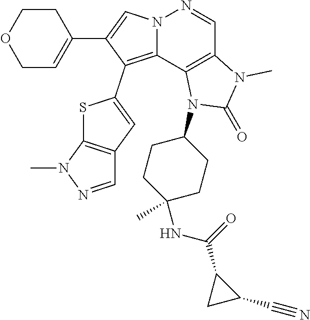 [SMILES: Cn1ncc2cc(-c3c(C4=CCOCC4)cn4ncc5c(c34)n([C@H]3CC[C@@](C)(NC(=O)[C@H]4C[C@H]4C#N)CC3)c(=O)n5C)sc21] |
| I-347-2 | 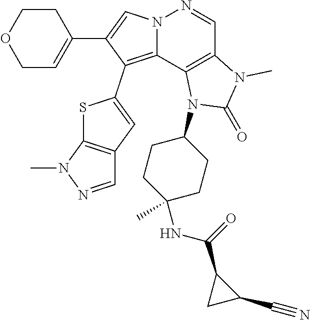 [SMILES: Cn1ncc2cc(-c3c(C4=CCOCC4)cn4ncc5c(c34)n([C@H]3CC[C@@](C)(NC(=O)[C@@H]4C[C@@H]4C#N)CC3)c(=O)n5C)sc21] |
| I-347′ | 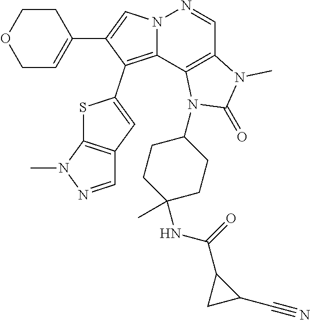 [SMILES: Cn1ncc2cc(-c3c(C4=CCOCC4)cn4ncc5c(c34)n(C3CCC(C)(NC(=O)C4CC4C#N)CC3)c(=O)n5C)sc21] |
| I-348-1 | 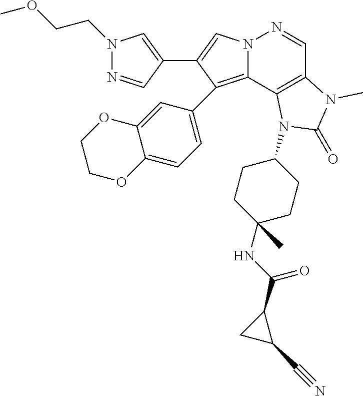 |
| I-348-2 | 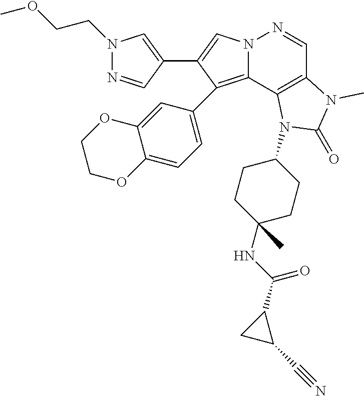 |
| I-348′ | 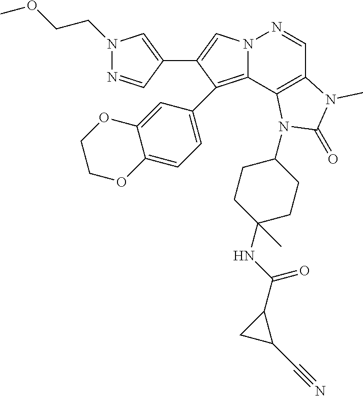 |
| I-349-1 | 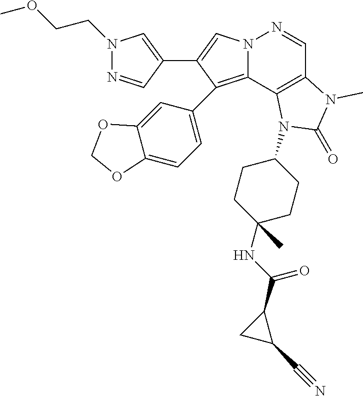 |
| I-349-2 | 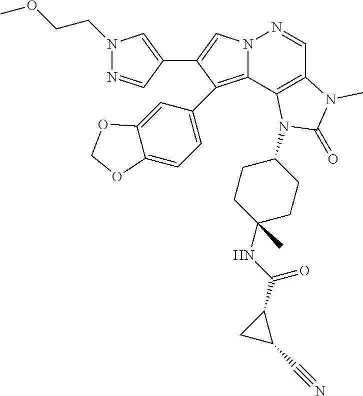 |
| I-349′ | 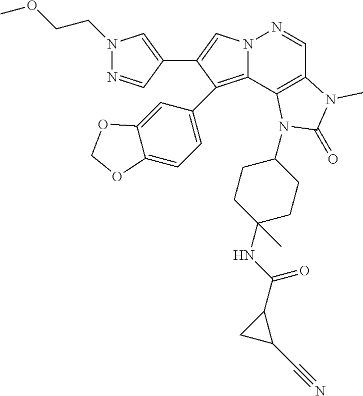 |
| I-350 | 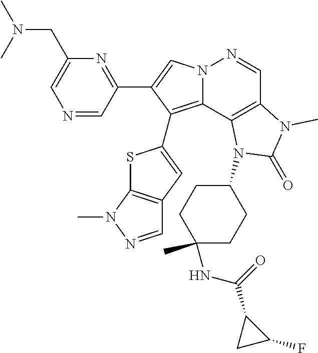 |
| I-350′ | 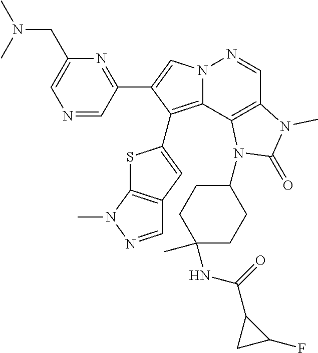 |
| I-351-1 | 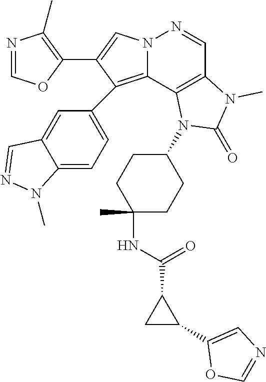 [SMILES: Cc1ncoc1-c1cn2ncc3c(c2c1-c1ccc2c(cnn2C)c1)n([C@H]1CC[C@@](C)(NC(=O)[C@H]2C[C@H]2c2cnco2)CC1)c(=O)n3C] |
| I-351-2 | 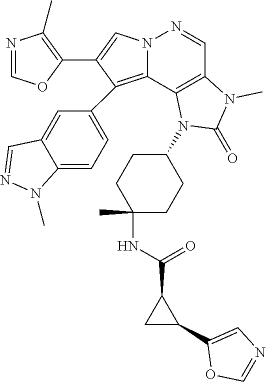 [SMILES: Cc1ncoc1-c1cn2ncc3c(c2c1-c1ccc2c(cnn2C)c1)n([C@H]1CC[C@@](C)(NC(=O)[C@@H]2C[C@@H]2c2cnco2)CC1)c(=O)n3C] |
| I-351′ | 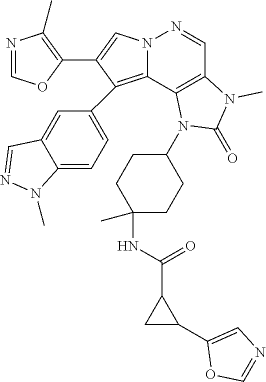 [SMILES: Cc1ncoc1-c1cn2ncc3c(c2c1-c1ccc2c(cnn2C)c1)n(C1CCC(C)(NC(=O)C2CC2c2cnco2)CC1)c(=O)n3C] |
| I-352-1 | 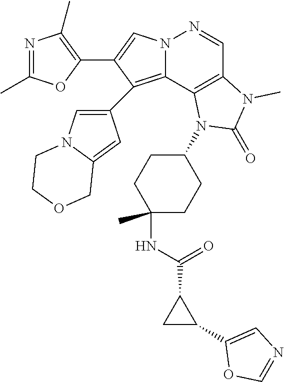 |
| I-352-2 | 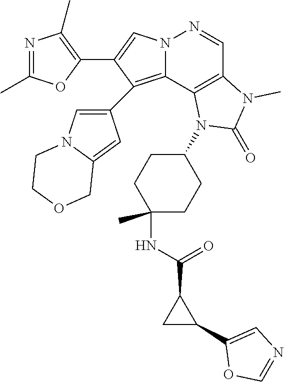 |
| I-352′ | 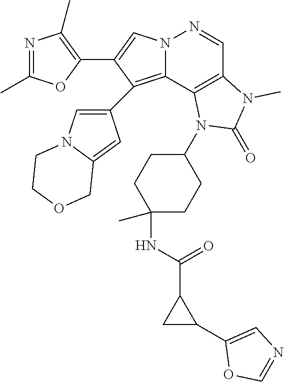 |
| I-353 | 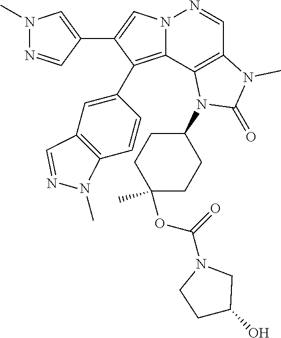 |
| I-353′ | 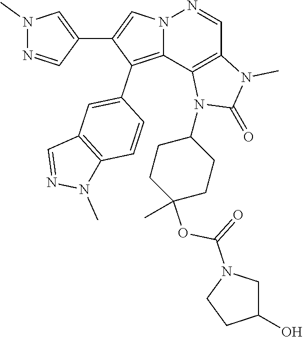 [SMILES: Cn1cc(-c2cn3ncc4c(c3c2-c2ccc3c(cnn3C)c2)n(C2CCC(C)(OC(=O)N3CCC(O)C3)CC2)c(=O)n4C)cn1] |
| I-354 | 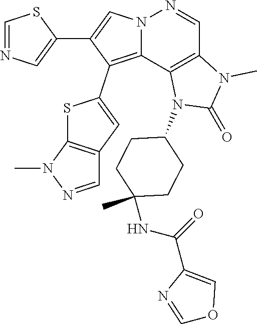 [SMILES: Cn1ncc2cc(-c3c(-c4cncs4)cn4ncc5c(c34)n([C@H]3CC[C@@](C)(NC(=O)c4cocn4)CC3)c(=O)n5C)sc21] |
| I-354′ | 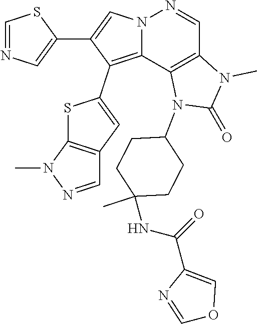 [SMILES: Cn1ncc2cc(-c3c(-c4cncs4)cn4ncc5c(c34)n(C3CCC(C)(NC(=O)c4cocn4)CC3)c(=O)n5C)sc21] |
| I-355 | 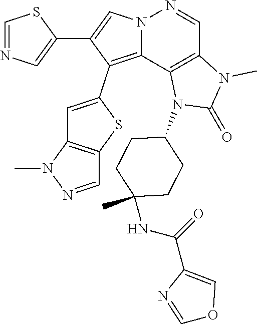 [SMILES: Cn1ncc2sc(-c3c(-c4cncs4)cn4ncc5c(c34)n([C@H]3CC[C@@](C)(NC(=O)c4cocn4)CC3)c(=O)n5C)cc21] |
| I-355′ | 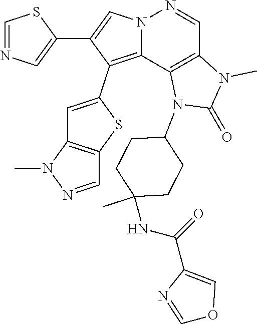 [SMILES: Cn1ncc2sc(-c3c(-c4cncs4)cn4ncc5c(c34)n(C3CCC(C)(NC(=O)c4cocn4)CC3)c(=O)n5C)cc21] |
| I-356 | 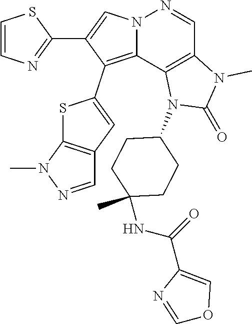 [SMILES: Cn1ncc2cc(-c3c(-c4nccs4)cn4ncc5c(c34)n([C@H]3CC[C@@](C)(NC(=O)c4cocn4)CC3)c(=O)n5C)sc21] |
| I-356′ | 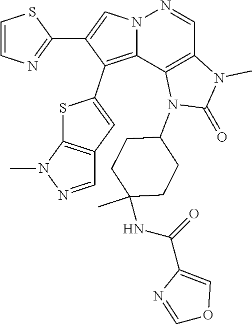 [SMILES: Cn1ncc2cc(-c3c(-c4nccs4)cn4ncc5c(c34)n(C3CCC(C)(NC(=O)c4cocn4)CC3)c(=O)n5C)sc21] |
| I-357 | 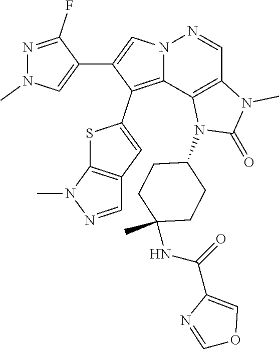 |
| I-357′ | 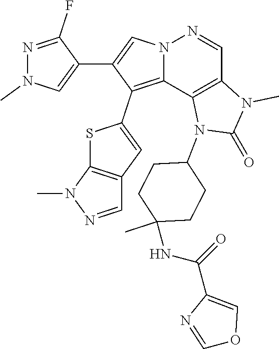 |
| I-358 | 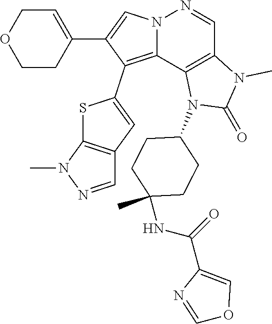 [SMILES: Cn1ncc2cc(-c3c(C4=CCOCC4)cn4ncc5c(c34)n([C@H]3CC[C@@](C)(NC(=O)c4cocn4)CC3)c(=O)n5C)sc21] |
| I-358′ | 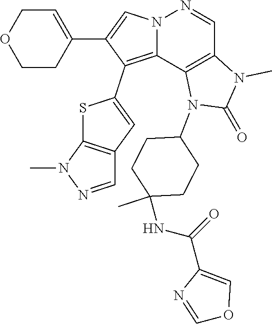 [SMILES: Cn1ncc2cc(-c3c(C4=CCOCC4)cn4ncc5c(c34)n(C3CCC(C)(NC(=O)c4cocn4)CC3)c(=O)n5C)sc21] |
| I-359 | 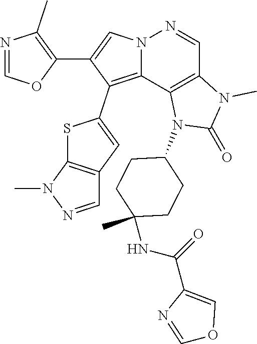 [SMILES: Cc1ncoc1-c1cn2ncc3c(c2c1-c1cc2cnn(C)c2s1)n([C@H]1CC[C@@](C)(NC(=O)c2cocn2)CC1)c(=O)n3C] |
| I-359′ | 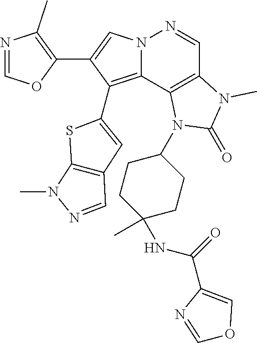 [SMILES: Cc1ncoc1-c1cn2ncc3c(c2c1-c1cc2cnn(C)c2s1)n(C1CCC(C)(NC(=O)c2cocn2)CC1)c(=O)n3C] |
| I-360 | 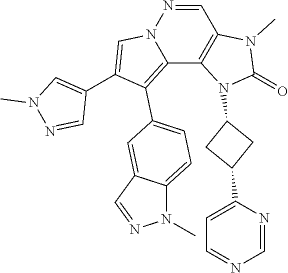 [SMILES: Cn1cc(-c2cn3ncc4c(c3c2-c2ccc3c(cnn3C)c2)n([C@H]2C[C@@H](c3ccncn3)C2)c(=O)n4C)cn1] |
| I-360′ | 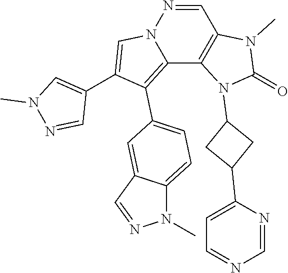 [SMILES: Cn1cc(-c2cn3ncc4c(c3c2-c2ccc3c(cnn3C)c2)n(C2CC(c3ccncn3)C2)c(=O)n4C)cn1] |
| I-361 | 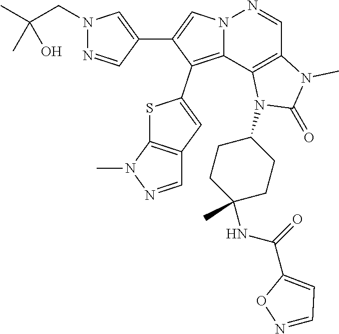 [SMILES: Cn1ncc2cc(-c3c(-c4cnn(CC(C)(C)O)c4)cn4ncc5c(c34)n([C@H]3CC[C@@](C)(NC(=O)c4ccno4)CC3)c(=O)n5C)sc21] |
| I-361′ | 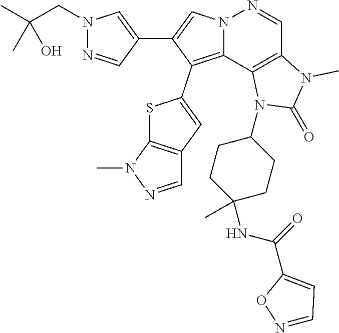 [SMILES: Cn1ncc2cc(-c3c(-c4cnn(CC(C)(C)O)c4)cn4ncc5c(c34)n(C3CCC(C)(NC(=O)c4ccno4)CC3)c(=O)n5C)sc21] |
| I-362 | 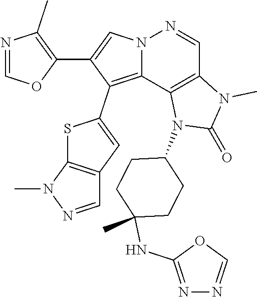 [SMILES: Cc1ncoc1-c1cn2ncc3c(c2c1-c1cc2cnn(C)c2s1)n([C@H]1CC[C@@](C)(Nc2nnco2)CC1)c(=O)n3C] |
| I-362′ | 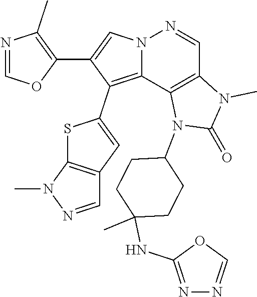 [SMILES: Cc1ncoc1-c1cn2ncc3c(c2c1-c1cc2cnn(C)c2s1)n(C1CCC(C)(Nc2nnco2)CC1)c(=O)n3C] |
| I-363 | 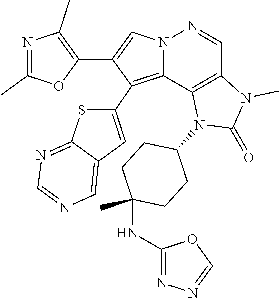 |
| I-363′ | 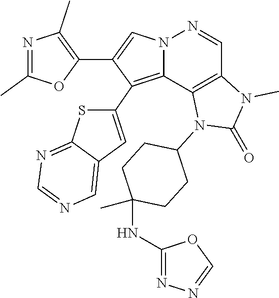 |
| I-364 | 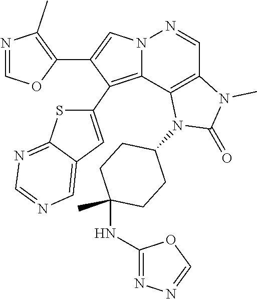 [SMILES: Cc1ncoc1-c1cn2ncc3c(c2c1-c1cc2cncnc2s1)n([C@H]1CC[C@@](C)(Nc2nnco2)CC1)c(=O)n3C] |
| I-364′ | 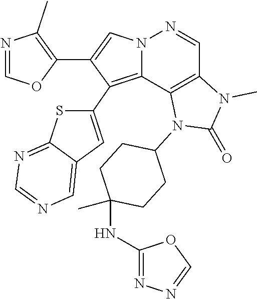 [SMILES: Cc1ncoc1-c1cn2ncc3c(c2c1-c1cc2cncnc2s1)n(C1CCC(C)(Nc2nnco2)CC1)c(=O)n3C] |
| I-365 | 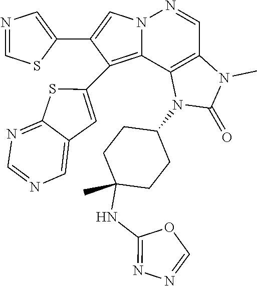 [SMILES: Cn1c(=O)n([C@H]2CC[C@@](C)(Nc3nnco3)CC2)c2c1cnn1cc(-c3cncs3)c(-c3cc4cncnc4s3)c21] |
| I-365′ | 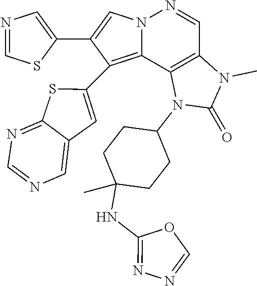 [SMILES: Cn1c(=O)n(C2CCC(C)(Nc3nnco3)CC2)c2c1cnn1cc(-c3cncs3)c(-c3cc4cncnc4s3)c21] |
| I-366 | 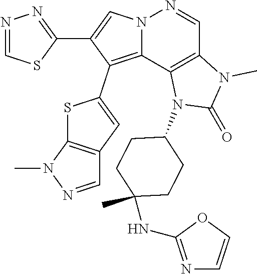 [SMILES: Cn1ncc2cc(-c3c(-c4nncs4)cn4ncc5c(c34)n([C@H]3CC[C@@](C)(Nc4ncco4)CC3)c(=O)n5C)sc21] |
| I-366′ | 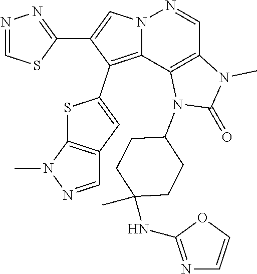 [SMILES: Cn1ncc2cc(-c3c(-c4nncs4)cn4ncc5c(c34)n(C3CCC(C)(Nc4ncco4)CC3)c(=O)n5C)sc21] |
| I-367 | 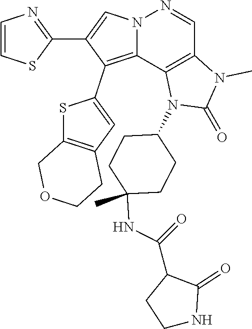 [SMILES: Cn1c(=O)n([C@H]2CC[C@@](C)(NC(=O)C3CCNC3=O)CC2)c2c1cnn1cc(-c3nccs3)c(-c3cc4c(s3)COCC4)c21] |
| I-367′ | 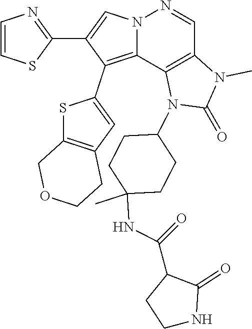 [SMILES: Cn1c(=O)n(C2CCC(C)(NC(=O)C3CCNC3=O)CC2)c2c1cnn1cc(-c3nccs3)c(-c3cc4c(s3)COCC4)c21] |
| I-368 | 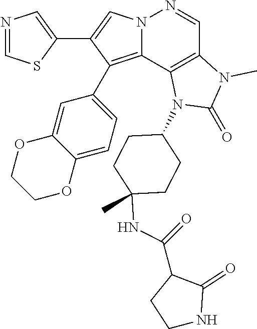 [SMILES: Cn1c(=O)n([C@H]2CC[C@@](C)(NC(=O)C3CCNC3=O)CC2)c2c1cnn1cc(-c3cncs3)c(-c3ccc4c(c3)OCCO4)c21] |
| I-368′ | 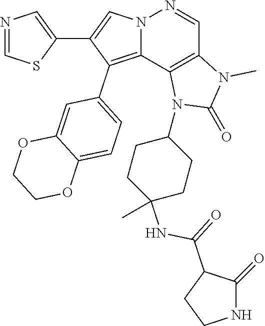 [SMILES: Cn1c(=O)n(C2CCC(C)(NC(=O)C3CCNC3=O)CC2)c2c1cnn1cc(-c3cncs3)c(-c3ccc4c(c3)OCCO4)c21] |
| I-369 | 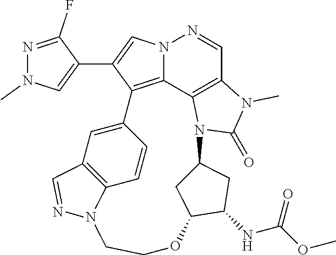 |
| I-369′ | 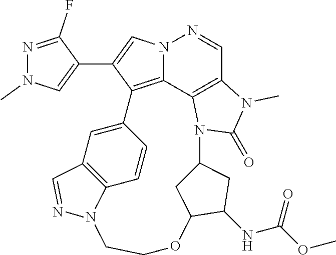 |
| I-370-1 | 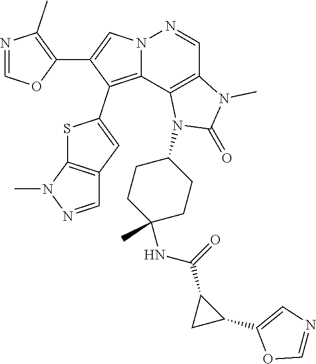 [SMILES: Cc1ncoc1-c1cn2ncc3c(c2c1-c1cc2cnn(C)c2s1)n([C@H]1CC[C@@](C)(NC(=O)[C@H]2C[C@H]2c2cnco2)CC1)c(=O)n3C] |
| I-370-2 | 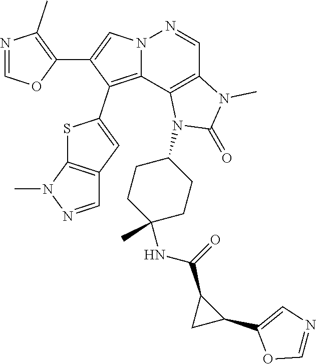 [SMILES: Cc1ncoc1-c1cn2ncc3c(c2c1-c1cc2cnn(C)c2s1)n([C@H]1CC[C@@](C)(NC(=O)[C@@H]2C[C@@H]2c2cnco2)CC1)c(=O)n3C] |
| I-370′ | 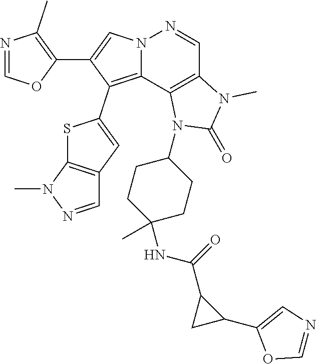 [SMILES: Cc1ncoc1-c1cn2ncc3c(c2c1-c1cc2cnn(C)c2s1)n(C1CCC(C)(NC(=O)C2CC2c2cnco2)CC1)c(=O)n3C] |
| I-371-1 | 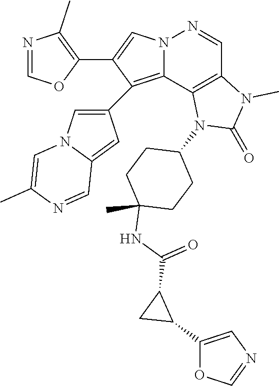 |
| I-371-2 | 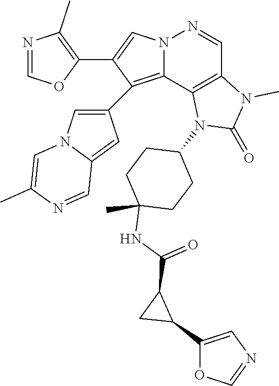 |
| I-371′ | 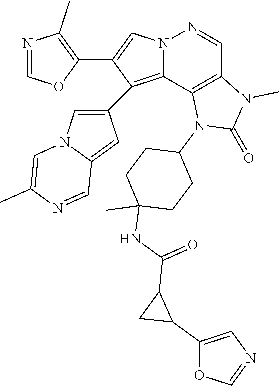 |
| I-372 | 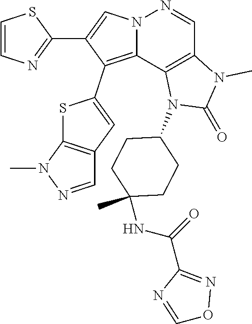 [SMILES: Cn1ncc2cc(-c3c(-c4nccs4)cn4ncc5c(c34)n([C@H]3CC[C@@](C)(NC(=O)c4ncon4)CC3)c(=O)n5C)sc21] |
| I-372′ | 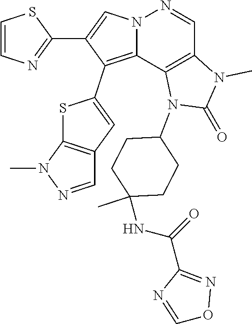 [SMILES: Cn1ncc2cc(-c3c(-c4nccs4)cn4ncc5c(c34)n(C3CCC(C)(NC(=O)c4ncon4)CC3)c(=O)n5C)sc21] |
| I-373 | 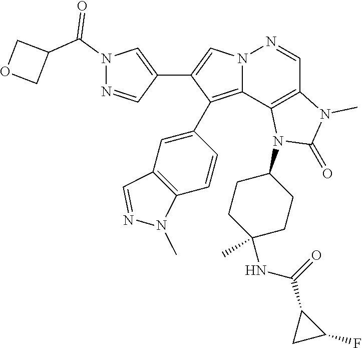 [SMILES: Cn1ncc2cc(-c3c(-c4cnn(C(=O)C5COC5)c4)cn4ncc5c(c34)n([C@H]3CC[C@@](C)(NC(=O)[C@H]4C[C@H]4F)CC3)c(=O)n5C)ccc21] |
| I-373′ | 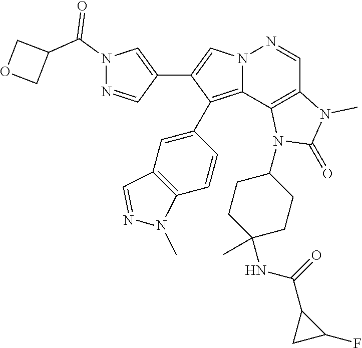 [SMILES: Cn1ncc2cc(-c3c(-c4cnn(C(=O)C5COC5)c4)cn4ncc5c(c34)n(C3CCC(C)(NC(=O)C4CC4F)CC3)c(=O)n5C)ccc21] |
| I-374 | 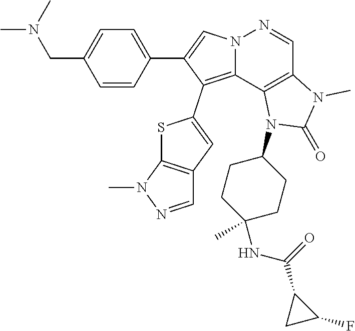 [SMILES: CN(C)Cc1ccc(-c2cn3ncc4c(c3c2-c2cc3cnn(C)c3s2)n([C@H]2CC[C@@](C)(NC(=O)[C@H]3C[C@H]3F)CC2)c(=O)n4C)cc1] |
| I-374′ | 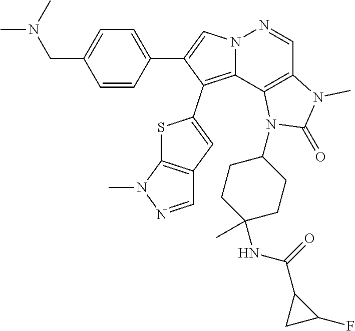 [SMILES: CN(C)Cc1ccc(-c2cn3ncc4c(c3c2-c2cc3cnn(C)c3s2)n(C2CCC(C)(NC(=O)C3CC3F)CC2)c(=O)n4C)cc1] |
| I-375 | 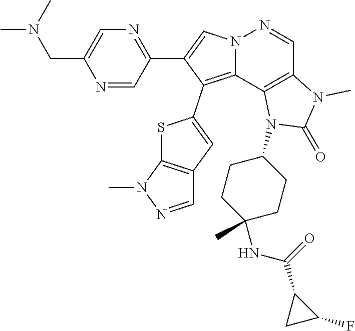 [SMILES: CN(C)Cc1cnc(-c2cn3ncc4c(c3c2-c2cc3cnn(C)c3s2)n([C@H]2CC[C@@](C)(NC(=O)[C@H]3C[C@H]3F)CC2)c(=O)n4C)cn1] |
| I-375′ | 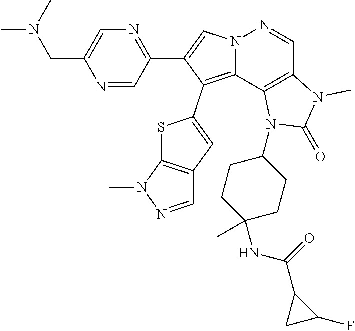 [SMILES: CN(C)Cc1cnc(-c2cn3ncc4c(c3c2-c2cc3cnn(C)c3s2)n(C2CCC(C)(NC(=O)C3CC3F)CC2)c(=O)n4C)cn1] |
| I-376 | 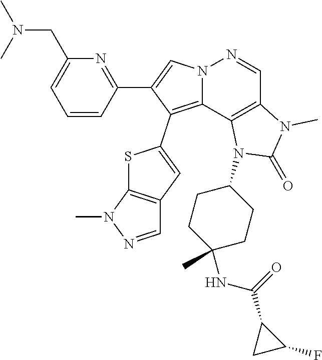 [SMILES: CN(C)Cc1cccc(-c2cn3ncc4c(c3c2-c2cc3cnn(C)c3s2)n([C@H]2CC[C@@](C)(NC(=O)[C@H]3C[C@H]3F)CC2)c(=O)n4C)n1] |
| I-376′ | 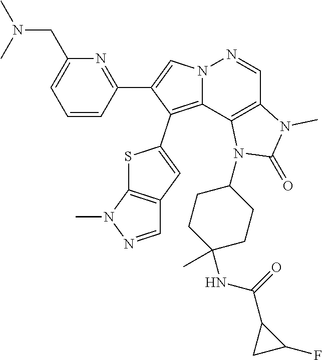 [SMILES: CN(C)Cc1cccc(-c2cn3ncc4c(c3c2-c2cc3cnn(C)c3s2)n(C2CCC(C)(NC(=O)C3CC3F)CC2)c(=O)n4C)n1] |
| I-377-1 | 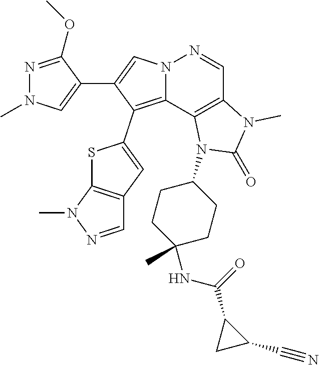 [SMILES: COc1nn(C)cc1-c1cn2ncc3c(c2c1-c1cc2cnn(C)c2s1)n([C@H]1CC[C@@](C)(NC(=O)[C@H]2C[C@H]2C#N)CC1)c(=O)n3C] |
| I-377-2 | 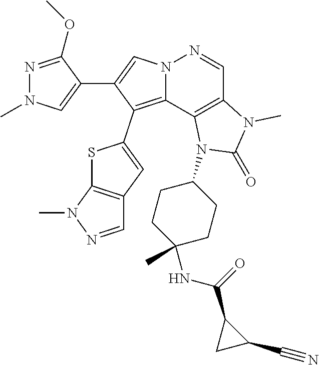 [SMILES: COc1nn(C)cc1-c1cn2ncc3c(c2c1-c1cc2cnn(C)c2s1)n([C@H]1CC[C@@](C)(NC(=O)[C@@H]2C[C@@H]2C#N)CC1)c(=O)n3C] |
| I-377′ | 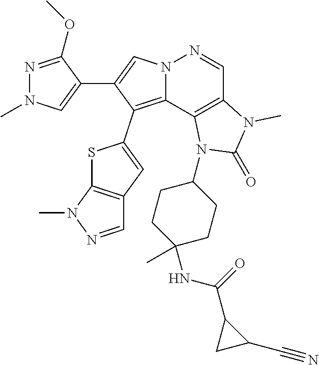 [SMILES: COc1nn(C)cc1-c1cn2ncc3c(c2c1-c1cc2cnn(C)c2s1)n(C1CCC(C)(NC(=O)C2CC2C#N)CC1)c(=O)n3C] |
| I-378 | 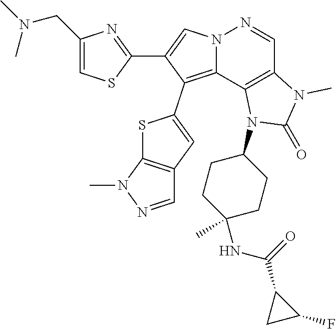 [SMILES: CN(C)Cc1csc(-c2cn3ncc4c(c3c2-c2cc3cnn(C)c3s2)n([C@H]2CC[C@@](C)(NC(=O)[C@H]3C[C@H]3F)CC2)c(=O)n4C)n1] |
| I-378′ | 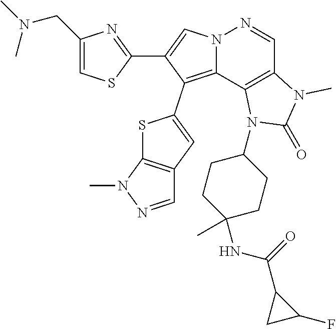 [SMILES: CN(C)Cc1csc(-c2cn3ncc4c(c3c2-c2cc3cnn(C)c3s2)n(C2CCC(C)(NC(=O)C3CC3F)CC2)c(=O)n4C)n1] |
| I-379 | 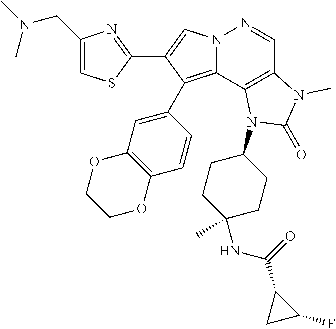 [SMILES: CN(C)Cc1csc(-c2cn3ncc4c(c3c2-c2ccc3c(c2)OCCO3)n([C@H]2CC[C@@](C)(NC(=O)[C@H]3C[C@H]3F)CC2)c(=O)n4C)n1] |
| I-379′ | 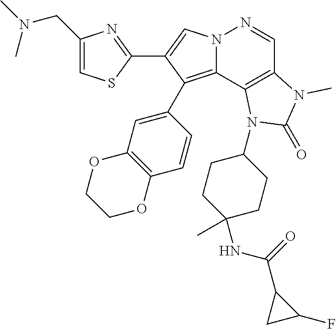 [SMILES: CN(C)Cc1csc(-c2cn3ncc4c(c3c2-c2ccc3c(c2)OCCO3)n(C2CCC(C)(NC(=O)C3CC3F)CC2)c(=O)n4C)n1] |
| I-380 | 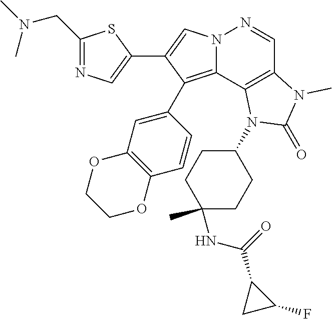 |
| I-380′ | 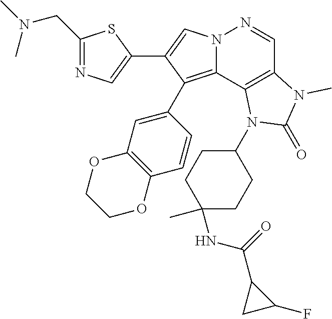 |
| I-381 | 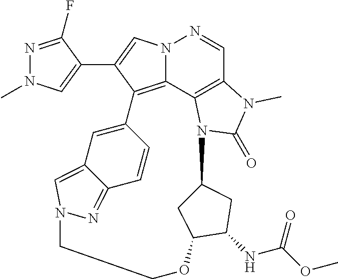 |
| I-381′ | 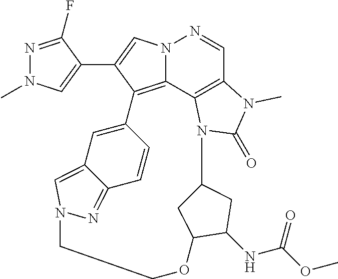 |

In some embodiments, the present disclosure provides a compound selected from Table 1B, or a pharmaceutically acceptable salt thereof.

TABLE 1B

| No. | Structure |
|---|---|
| I-214 | 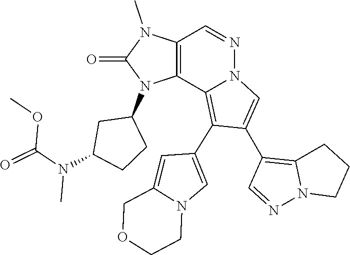 [SMILES: COC(=O)N(C)[C@H]1CC[C@H](n2c(=O)n(C)c3cnn4cc(-c5cnn6c5CCC6)c(-c5cc6n(c5)CCOC6)c4c32)C1] |
| I-214′ | 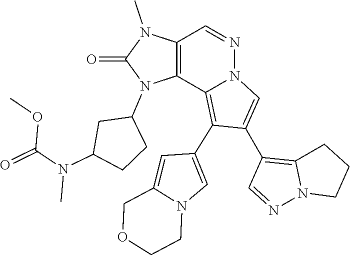 [SMILES: COC(=O)N(C)C1CCC(n2c(=O)n(C)c3cnn4cc(-c5cnn6c5CCC6)c(-c5cc6n(c5)CCOC6)c4c32)C1] |
| I-204 | 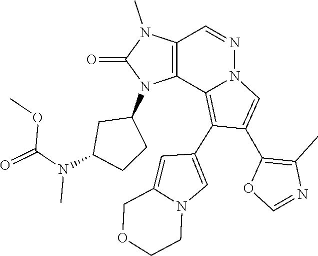 [SMILES: COC(=O)N(C)[C@H]1CC[C@H](n2c(=O)n(C)c3cnn4cc(-c5ocnc5C)c(-c5cc6n(c5)CCOC6)c4c32)C1] |
| I-204′ | 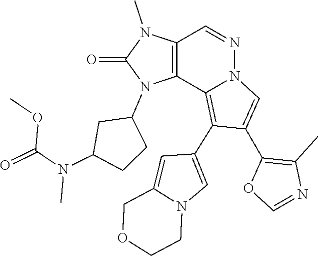 [SMILES: COC(=O)N(C)C1CCC(n2c(=O)n(C)c3cnn4cc(-c5ocnc5C)c(-c5cc6n(c5)CCOC6)c4c32)C1] |
| I-201 | 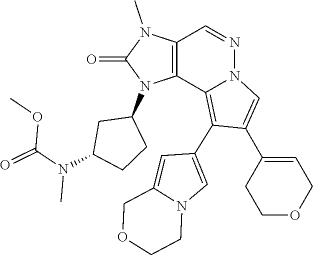 [SMILES: COC(=O)N(C)[C@H]1CC[C@H](n2c(=O)n(C)c3cnn4cc(C5=CCOCC5)c(-c5cc6n(c5)CCOC6)c4c32)C1] |
| I-201′ | 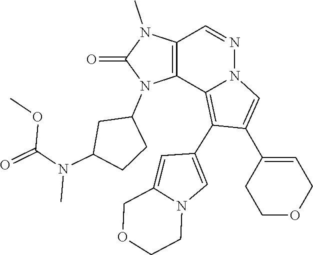 [SMILES: COC(=O)N(C)C1CCC(n2c(=O)n(C)c3cnn4cc(C5=CCOCC5)c(-c5cc6n(c5)CCOC6)c4c32)C1] |
| I-203 | 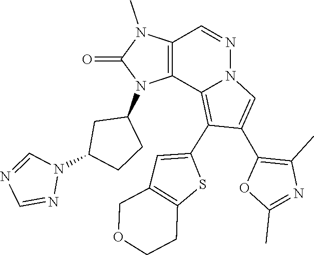 [SMILES: Cc1nc(C)c(-c2cn3ncc4c(c3c2-c2cc3c(s2)CCOC3)n([C@H]2CC[C@H](n3cncn3)C2)c(=O)n4C)o1] |
| I-203′ | 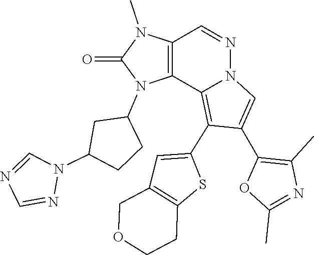 [SMILES: Cc1nc(C)c(-c2cn3ncc4c(c3c2-c2cc3c(s2)CCOC3)n(C2CCC(n3cncn3)C2)c(=O)n4C)o1] |
| I-211 | 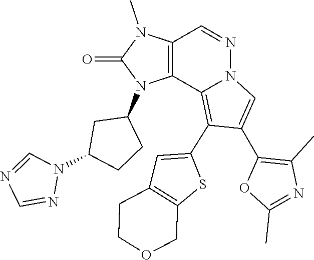 [SMILES: Cc1nc(C)c(-c2cn3ncc4c(c3c2-c2cc3c(s2)COCC3)n([C@H]2CC[C@H](n3cncn3)C2)c(=O)n4C)o1] |
| I-211′ | 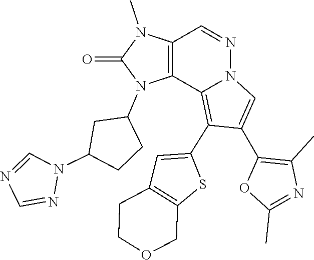 [SMILES: Cc1nc(C)c(-c2cn3ncc4c(c3c2-c2cc3c(s2)COCC3)n(C2CCC(n3cncn3)C2)c(=O)n4C)o1] |
| I-212 | 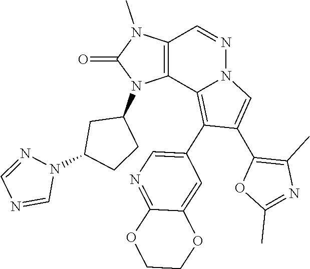 [SMILES: Cc1nc(C)c(-c2cn3ncc4c(c3c2-c2cnc3c(c2)OCCO3)n([C@H]2CC[C@H](n3cncn3)C2)c(=O)n4C)o1] |
| I-212′ | 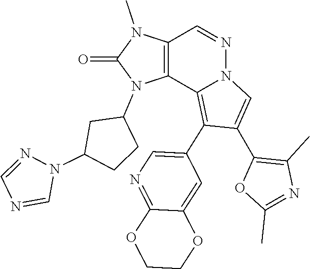 [SMILES: Cc1nc(C)c(-c2cn3ncc4c(c3c2-c2cnc3c(c2)OCCO3)n(C2CCC(n3cncn3)C2)c(=O)n4C)o1] |
| I-198 | 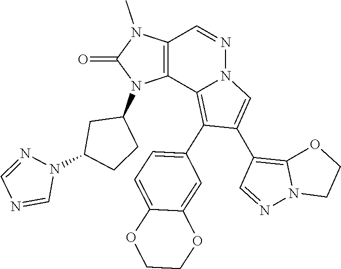 [SMILES: Cn1c(=O)n([C@H]2CC[C@H](n3cncn3)C2)c2c1cnn1cc(-c3cnn4c3OCC4)c(-c3ccc4c(c3)OCCO4)c21] |
| I-198′ | 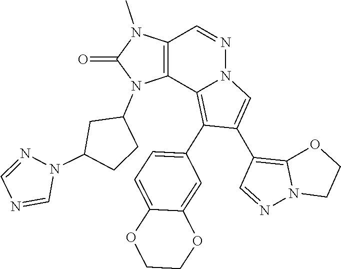 [SMILES: Cn1c(=O)n(C2CCC(n3cncn3)C2)c2c1cnn1cc(-c3cnn4c3OCC4)c(-c3ccc4c(c3)OCCO4)c21] |

In some embodiments, the present disclosure provides a compound selected from Table 1C, or a pharmaceutically acceptable salt thereof.

TABLE 1C

| No. | Structure |
|---|---|
| I-1 | 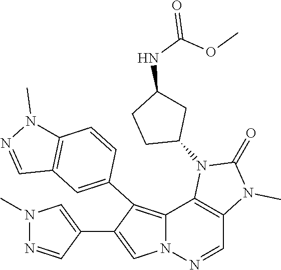 |
| I-2 | 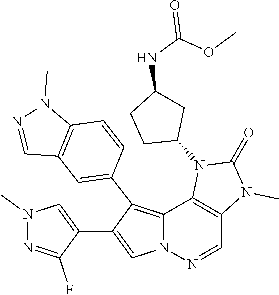 |
| I-3 | 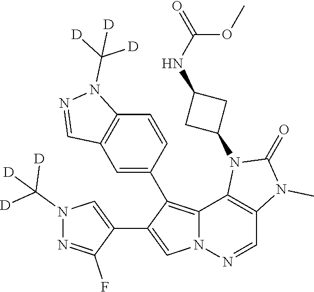 [SMILES: [2H]C([2H])([2H])n1cc(-c2cn3ncc4c(c3c2-c2ccc3c(cnn3C([2H])([2H])[2H])c2)n([C@H]2C[C@@H](NC(=O)OC)C2)c(=O)n4C)c(F)n1] |
| I-4′ | 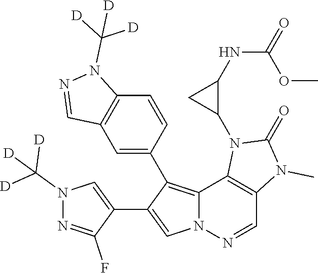 [SMILES: [2H]C([2H])([2H])n1cc(-c2cn3ncc4c(c3c2-c2ccc3c(cnn3C([2H])([2H])[2H])c2)n(C2CC2NC(=O)OC)c(=O)n4C)c(F)n1] |
| I-4-1 | 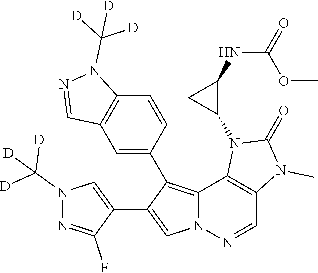 [SMILES: [2H]C([2H])([2H])n1cc(-c2cn3ncc4c(c3c2-c2ccc3c(cnn3C([2H])([2H])[2H])c2)n([C@@H]2C[C@H]2NC(=O)OC)c(=O)n4C)c(F)n1] |
| I-4-2 | 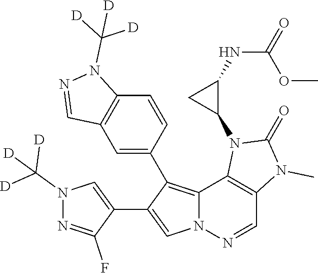 [SMILES: [2H]C([2H])([2H])n1cc(-c2cn3ncc4c(c3c2-c2ccc3c(cnn3C([2H])([2H])[2H])c2)n([C@H]2C[C@@H]2NC(=O)OC)c(=O)n4C)c(F)n1] |
| C5 | 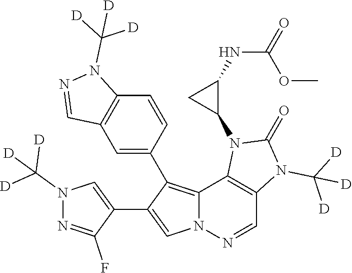 [SMILES: [2H]C([2H])([2H])n1cc(-c2cn3ncc4c(c3c2-c2ccc3c(cnn3C([2H])([2H])[2H])c2)n([C@H]2C[C@@H]2NC(=O)OC)c(=O)n4C([2H])([2H])[2H])c(F)n1] |
| C6 | 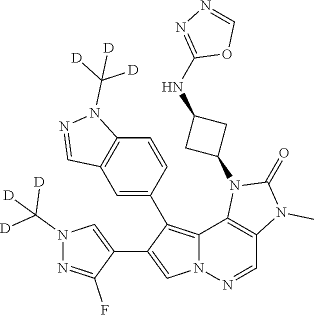 [SMILES: [2H]C([2H])([2H])n1cc(-c2cn3ncc4c(c3c2-c2ccc3c(cnn3C([2H])([2H])[2H])c2)n([C@H]2C[C@@H](Nc3nnco3)C2)c(=O)n4C)c(F)n1] |
| C7 | 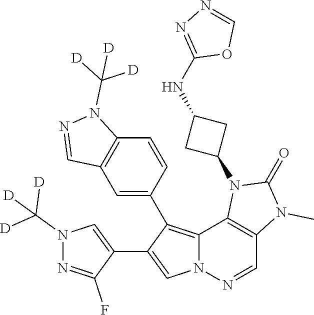 [SMILES: [2H]C([2H])([2H])n1cc(-c2cn3ncc4c(c3c2-c2ccc3c(cnn3C([2H])([2H])[2H])c2)n([C@H]2C[C@H](Nc3nnco3)C2)c(=O)n4C)c(F)n1] |
| I-6 | 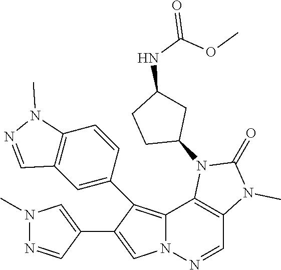 |
| C9 | 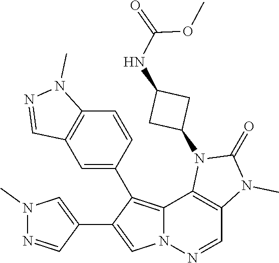 |
| C10 | 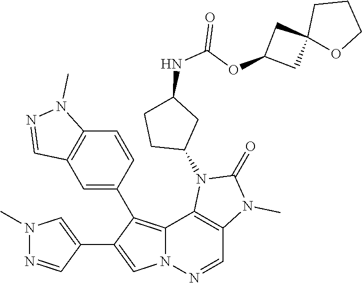 [SMILES: Cn1cc(-c2cn3ncc4c(c3c2-c2ccc3c(cnn3C)c2)n([C@@H]2CC[C@@H](NC(=O)O[C@H]3C[C@]5(CCCO5)C3)C2)c(=O)n4C)cn1] |
| I-5 | 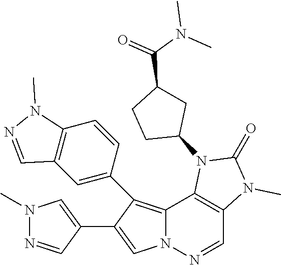 |
| C12 | 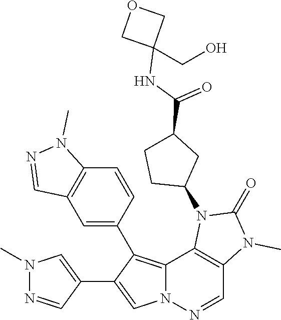 |
| C13 | 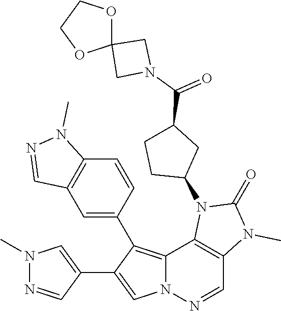 |
| C14 | 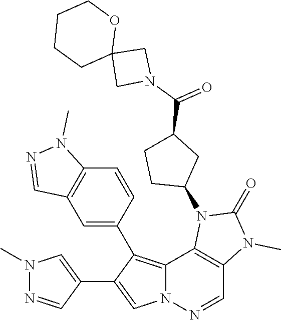 |
| C15 | 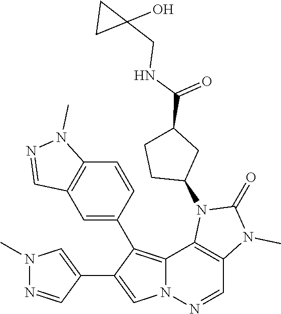 |
| C16 | 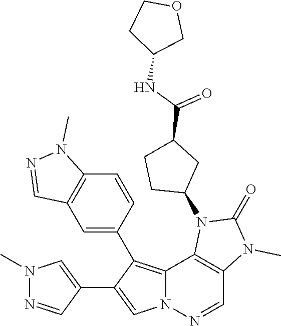 |
| C17 | 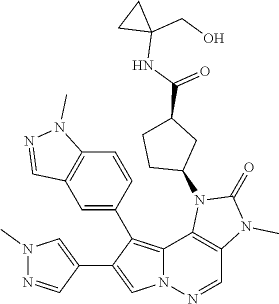 |
| I-14 | 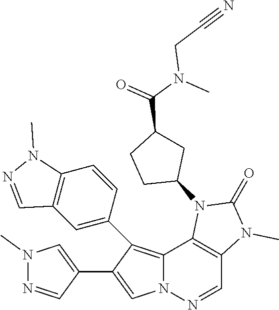 |
| C19 | 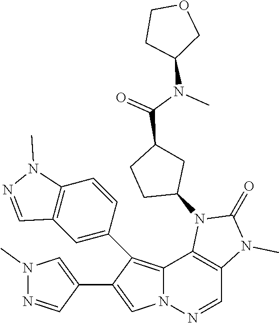 |
| C20 | 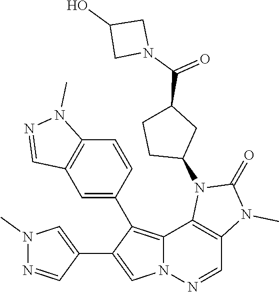 |
| C21 | 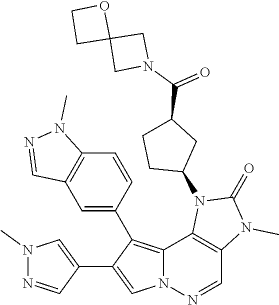 |
| C22 | 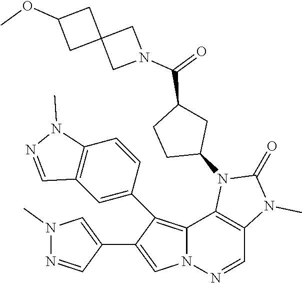 |
| C23 | 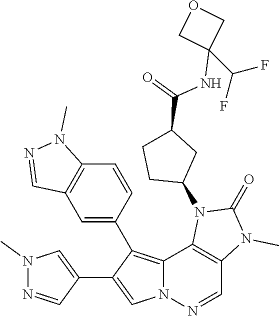 |
| C24 | 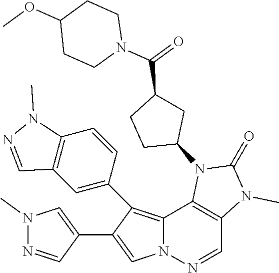 |
| C25 | 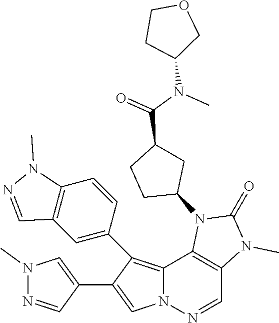 |
| I-8 | 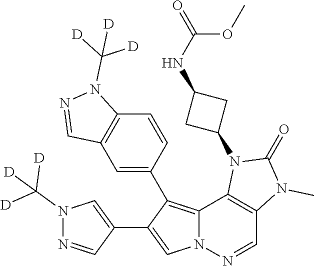 [SMILES: [2H]C([2H])([2H])n1cc(-c2cn3ncc4c(c3c2-c2ccc3c(cnn3C([2H])([2H])[2H])c2)n([C@H]2C[C@@H](NC(=O)OC)C2)c(=O)n4C)cn1] |
| C27 | 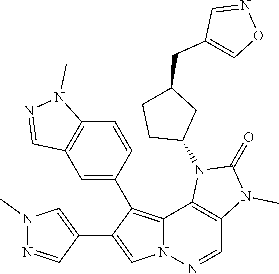 |
| C28 | 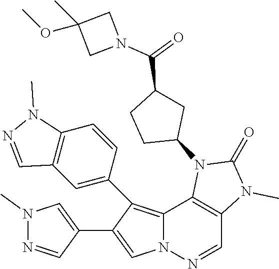 |
| C29 | 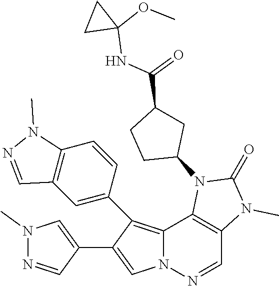 |
| I-24 | 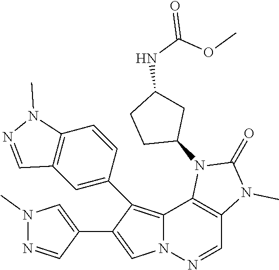 |
| C31 | 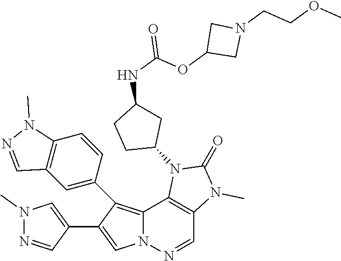 |
| C32 | 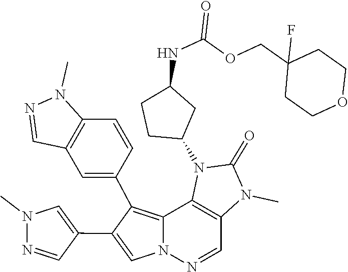 [SMILES: Cn1cc(-c2cn3ncc4c(c3c2-c2ccc3c(cnn3C)c2)n([C@@H]2CC[C@@H](NC(=O)OCC3(F)CCOCC3)C2)c(=O)n4C)cn1] |
| C33 | 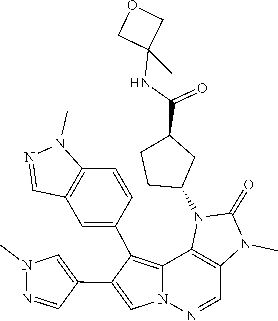 |
| C34 | 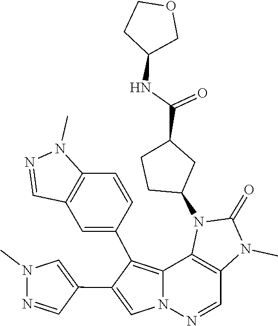 |
| C35 | 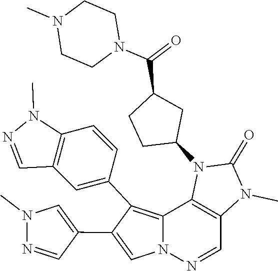 |
| C36 | 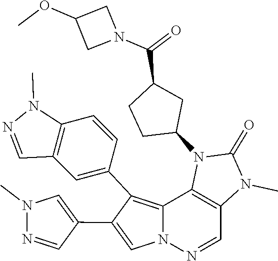 |
| C37 | 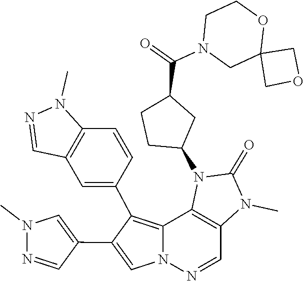 [SMILES: Cn1cc(-c2cn3ncc4c(c3c2-c2ccc3c(cnn3C)c2)n([C@H]2CC[C@@H](C(=O)N3CCOC5(COC5)C3)C2)c(=O)n4C)cn1] |
| C38 | 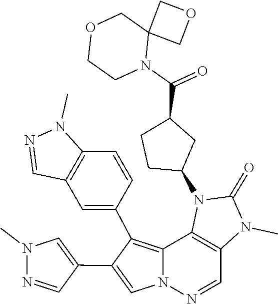 |
| C39 | 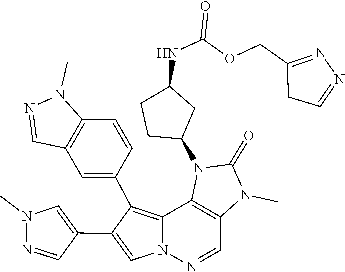 [SMILES: Cn1cc(-c2cn3ncc4c(c3c2-c2ccc3c(cnn3C)c2)n([C@H]2CC[C@@H](NC(=O)OCC3=NN=CC3)C2)c(=O)n4C)cn1] |
| C40 | 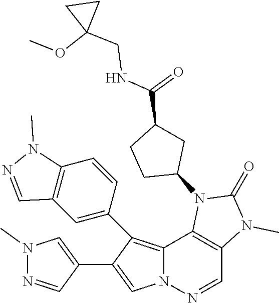 |
| C41 | 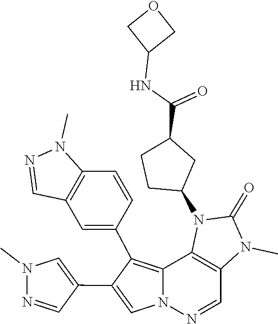 |
| C42 | 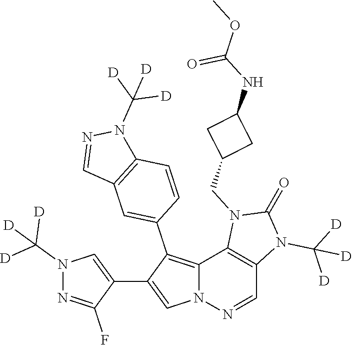 [SMILES: [2H]C([2H])([2H])n1cc(-c2cn3ncc4c(c3c2-c2ccc3c(cnn3C([2H])([2H])[2H])c2)n(C[C@H]2C[C@H](NC(=O)OC)C2)c(=O)n4C([2H])([2H])[2H])c(F)n1] |
| C43 | 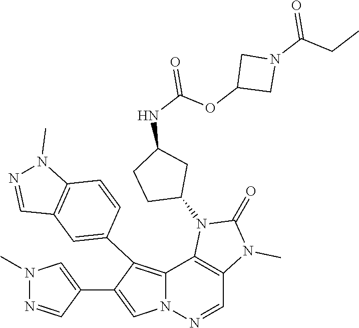 [SMILES: CCC(=O)N1CC(OC(=O)N[C@@H]2CC[C@@H](n3c(=O)n(C)c4cnn5cc(-c6cnn(C)c6)c(-c6ccc7c(cnn7C)c6)c5c43)C2)C1] |
| C44 | 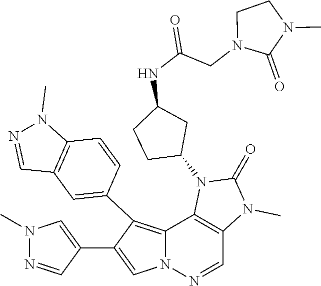 |
| C45 | 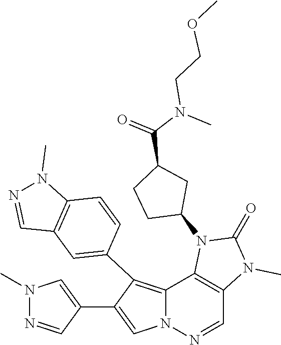 |
| C46 | 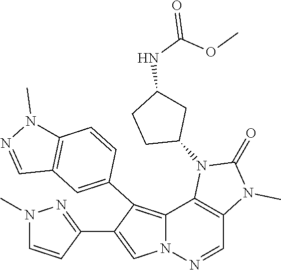 |
| C47 | 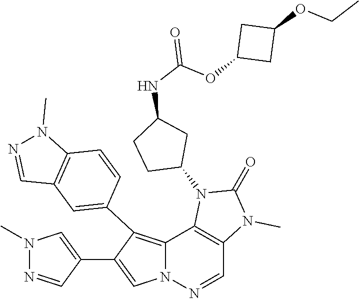 [SMILES: CCO[C@H]1C[C@H](OC(=O)N[C@@H]2CC[C@@H](n3c(=O)n(C)c4cnn5cc(-c6cnn(C)c6)c(-c6ccc7c(cnn7C)c6)c5c43)C2)C1] |
| C48 | 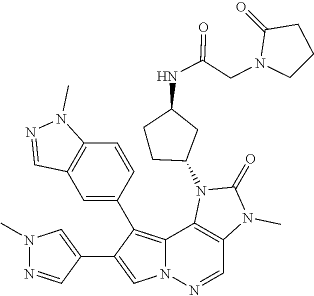 [SMILES: Cn1cc(-c2cn3ncc4c(c3c2-c2ccc3c(cnn3C)c2)n([C@@H]2CC[C@@H](NC(=O)CN3CCCC3=O)C2)c(=O)n4C)cn1] |
| C49 | 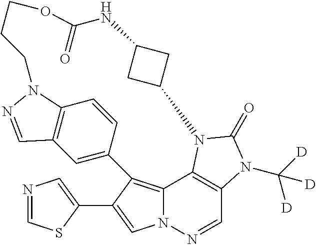 |
| C50 | 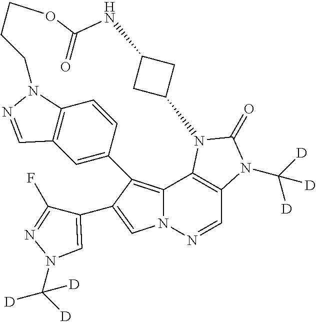 |
| I-49 | 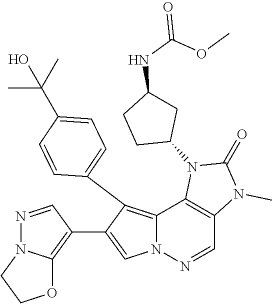 [SMILES: COC(=O)N[C@@H]1CC[C@@H](n2c(=O)n(C)c3cnn4cc(-c5cnn6c5OCC6)c(-c5ccc(C(C)(C)O)cc5)c4c32)C1] |
| I-16 | 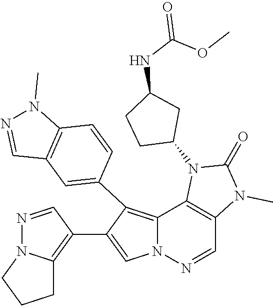 [SMILES: COC(=O)N[C@@H]1CC[C@@H](n2c(=O)n(C)c3cnn4cc(-c5cnn6c5CCC6)c(-c5ccc6c(cnn6C)c5)c4c32)C1] |
| C53 | 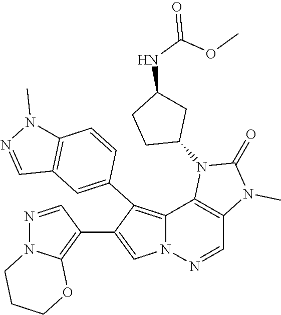 [SMILES: COC(=O)N[C@@H]1CC[C@@H](n2c(=O)n(C)c3cnn4cc(-c5cnn6c5OCCC6)c(-c5ccc6c(cnn6C)c5)c4c32)C1] |
| C54 | 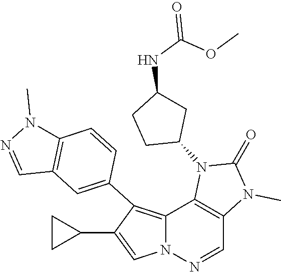 [SMILES: COC(=O)N[C@@H]1CC[C@@H](n2c(=O)n(C)c3cnn4cc(C5CC5)c(-c5ccc6c(cnn6C)c5)c4c32)C1] |
| C55 | 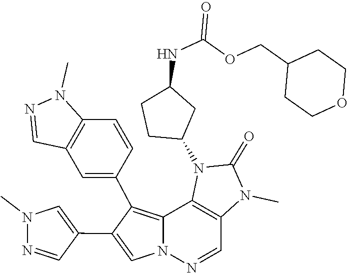 [SMILES: Cn1cc(-c2cn3ncc4c(c3c2-c2ccc3c(cnn3C)c2)n([C@@H]2CC[C@@H](NC(=O)OCC3CCOCC3)C2)c(=O)n4C)cn1] |
| C56 | 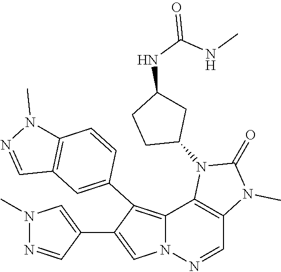 |
| C57 | 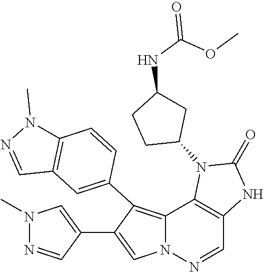 [SMILES: COC(=O)N[C@@H]1CC[C@@H](n2c(=O)[nH]c3cnn4cc(-c5cnn(C)c5)c(-c5ccc6c(cnn6C)c5)c4c32)C1] |
| I-12 | 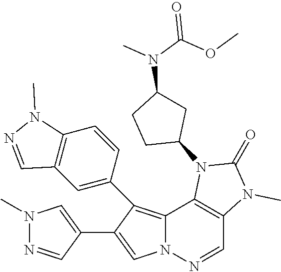 |
| I-9 | 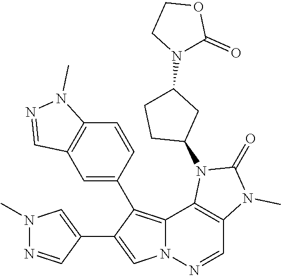 |
| I-10 | 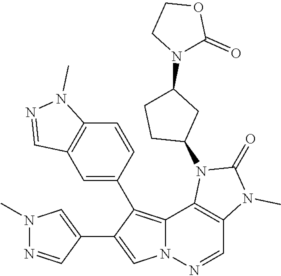 |
| C61 | 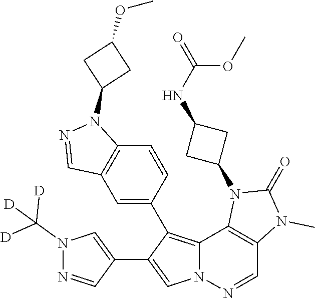 [SMILES: [2H]C([2H])([2H])n1cc(-c2cn3ncc4c(c3c2-c2ccc3c(cnn3[C@H]3C[C@H](OC)C3)c2)n([C@H]2C[C@@H](NC(=O)OC)C2)c(=O)n4C)cn1] |
| C62 | 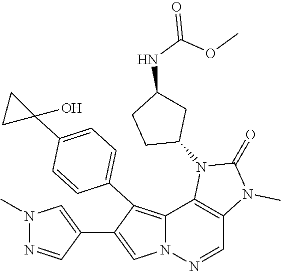 |
| C63 | 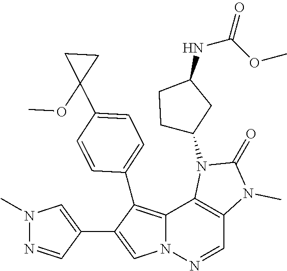 |
| C64 | 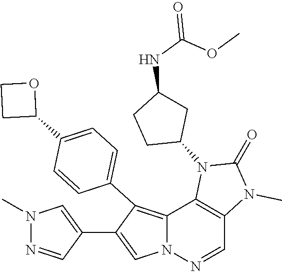 [SMILES: COC(=O)N[C@@H]1CC[C@@H](n2c(=O)n(C)c3cnn4cc(-c5cnn(C)c5)c(-c5ccc([C@@H]6CCO6)cc5)c4c32)C1] |
| C65 | 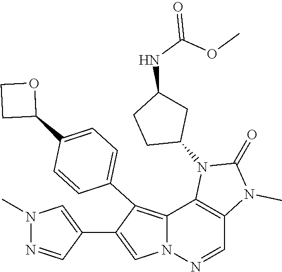 [SMILES: COC(=O)N[C@@H]1CC[C@@H](n2c(=O)n(C)c3cnn4cc(-c5cnn(C)c5)c(-c5ccc([C@H]6CCO6)cc5)c4c32)C1] |
| C66 | 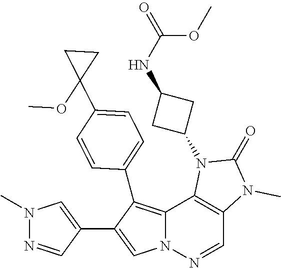 |
| C67 | 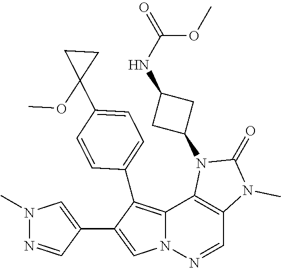 |
| C68 | 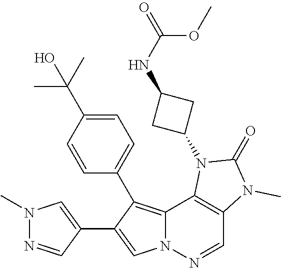 |
| C69 | 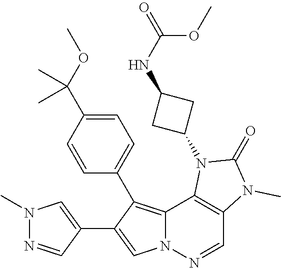 |
| C70 | 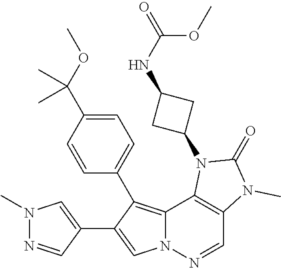 |
| C71 | 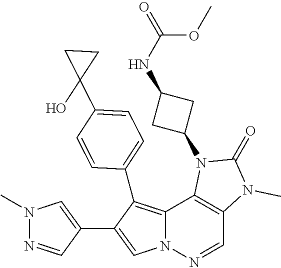 |
| C72 | 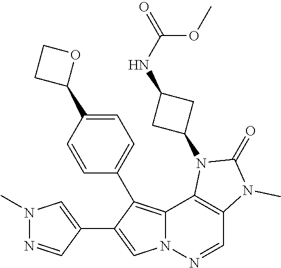 |
| C73 | 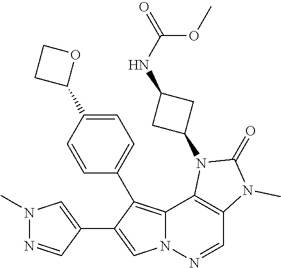 |
| C74 | 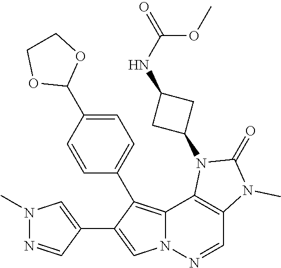 |
| C75 | 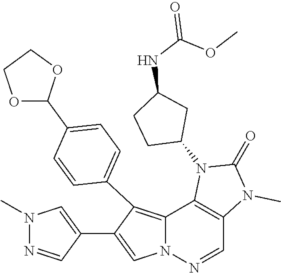 |
| C76 | 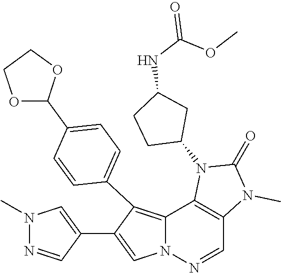 |
| C77 | 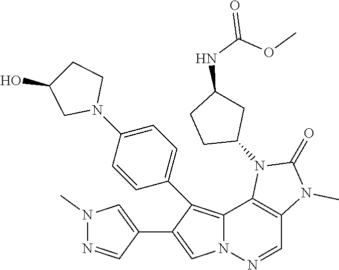 [SMILES: COC(=O)N[C@@H]1CC[C@@H](n2c(=O)n(C)c3cnn4cc(-c5cnn(C)c5)c(-c5ccc(N6CC[C@H](O)C6)cc5)c4c32)C1] |
| C78 | 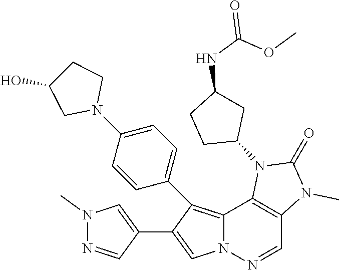 [SMILES: COC(=O)N[C@@H]1CC[C@@H](n2c(=O)n(C)c3cnn4cc(-c5cnn(C)c5)c(-c5ccc(N6CC[C@@H](O)C6)cc5)c4c32)C1] |
| C79 | 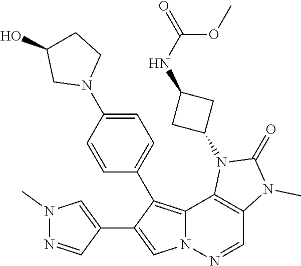 [SMILES: COC(=O)N[C@H]1C[C@H](n2c(=O)n(C)c3cnn4cc(-c5cnn(C)c5)c(-c5ccc(N6CC[C@H](O)C6)cc5)c4c32)C1] |
| C80 | 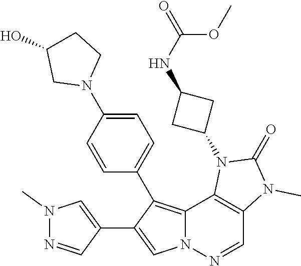 [SMILES: COC(=O)N[C@H]1C[C@H](n2c(=O)n(C)c3cnn4cc(-c5cnn(C)c5)c(-c5ccc(N6CC[C@@H](O)C6)cc5)c4c32)C1] |
| C81 | 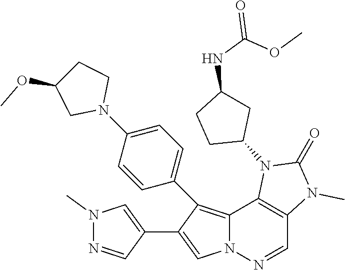 |
| C82 | 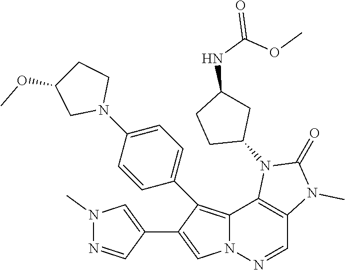 |
| C83 | 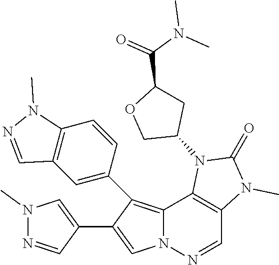 |
| C84 | 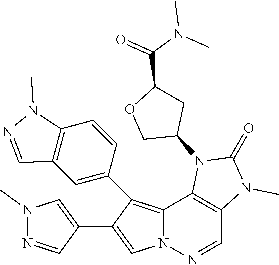 |
| C85 | 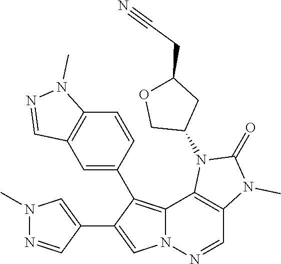 |
| C86 | 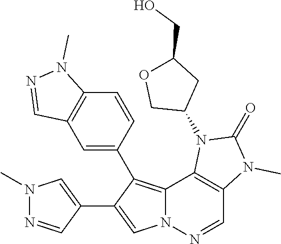 |
| I-42 | 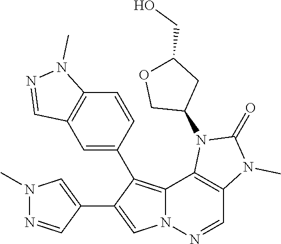 |
| C88 | 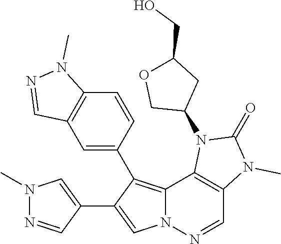 |
| C89 | 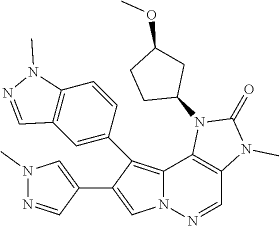 |
| I-37-1 | 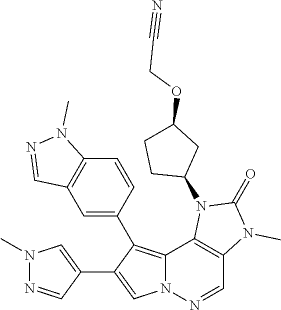 |
| C91 | 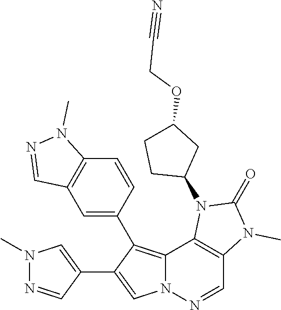 |
| C92 | 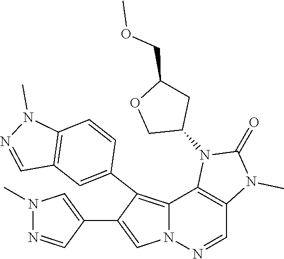 |
| C93 | 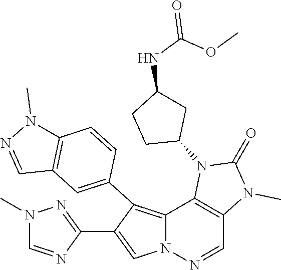 |
| C94 | 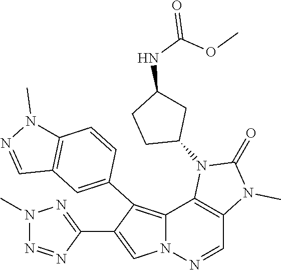 |
| C95 | 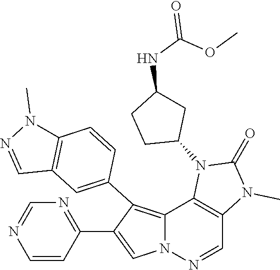 [SMILES: COC(=O)N[C@@H]1CC[C@@H](n2c(=O)n(C)c3cnn4cc(-c5ccncn5)c(-c5ccc6c(cnn6C)c5)c4c32)C1] |
| C96 | 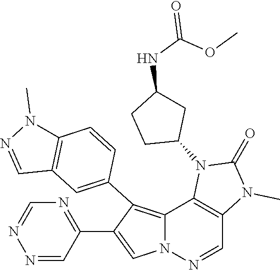 [SMILES: COC(=O)N[C@@H]1CC[C@@H](n2c(=O)n(C)c3cnn4cc(-c5cnncn5)c(-c5ccc6c(cnn6C)c5)c4c32)C1] |
| C97 | 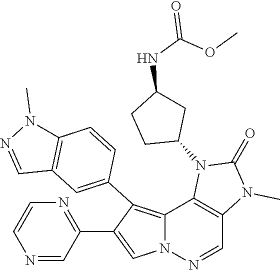 [SMILES: COC(=O)N[C@@H]1CC[C@@H](n2c(=O)n(C)c3cnn4cc(-c5cnccn5)c(-c5ccc6c(cnn6C)c5)c4c32)C1] |
| C98 | 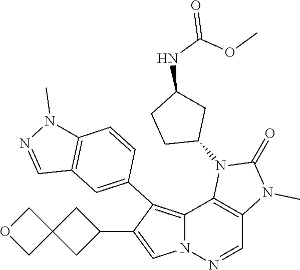 [SMILES: COC(=O)N[C@@H]1CC[C@@H](n2c(=O)n(C)c3cnn4cc(C5CC6(COC6)C5)c(-c5ccc6c(cnn6C)c5)c4c32)C1] |
| C99 | 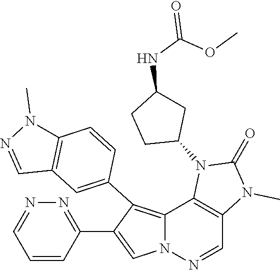 [SMILES: COC(=O)N[C@@H]1CC[C@@H](n2c(=O)n(C)c3cnn4cc(-c5cccnn5)c(-c5ccc6c(cnn6C)c5)c4c32)C1] |
| C100 | 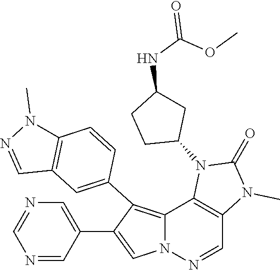 [SMILES: COC(=O)N[C@@H]1CC[C@@H](n2c(=O)n(C)c3cnn4cc(-c5cncnc5)c(-c5ccc6c(cnn6C)c5)c4c32)C1] |
| C101 | 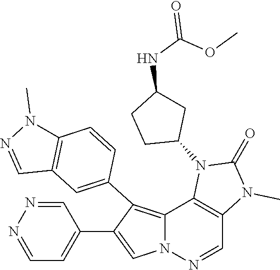 [SMILES: COC(=O)N[C@@H]1CC[C@@H](n2c(=O)n(C)c3cnn4cc(-c5ccnnc5)c(-c5ccc6c(cnn6C)c5)c4c32)C1] |
| C102 | 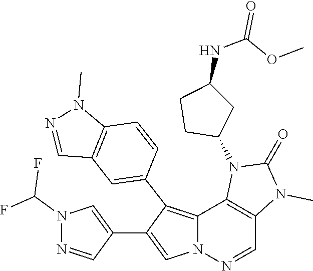 [SMILES: COC(=O)N[C@@H]1CC[C@@H](n2c(=O)n(C)c3cnn4cc(-c5cnn(C(F)F)c5)c(-c5ccc6c(cnn6C)c5)c4c32)C1] |
| C103 | 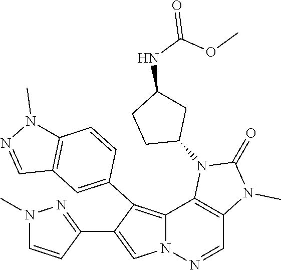 |
| C104 | 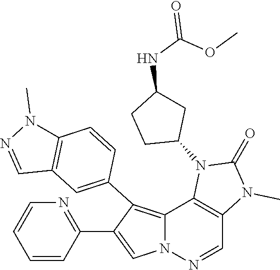 [SMILES: COC(=O)N[C@@H]1CC[C@@H](n2c(=O)n(C)c3cnn4cc(-c5ccccn5)c(-c5ccc6c(cnn6C)c5)c4c32)C1] |
| C105 | 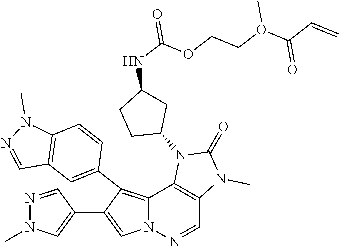 |
| C106 | 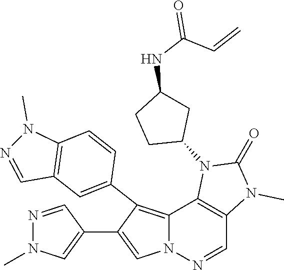 |

Structures depicted herein are meant to include all stereoisomeric (e.g., enantiomeric or diasteromeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomic, and geometric (or conformational) mixtures of provided compounds are within the scope of the disclosure. For example, in some cases, Tables 1A-1C show one or more stereoisomers of a compound, and unless otherwise indicated, represent each stereoisomer alone and/or as a mixture. Unless otherwise stated, all tautomeric forms of provided compounds are within the scope of the disclosure.

In some embodiments, the present disclosure encompasses the recognition that provided compounds display certain desirable characteristics, e.g., as compared to other known compounds. For example, in some embodiments, provided compounds are more potent in one or more biochemical or cellular assays (e.g., the JAK2 Binding Assay or SET2-pSTAT5 Cellular Assay described herein) and/or have one or more other characteristics that make them more suitable for drug development, such as better selectivity over other kinases (e.g., over JAK1, JAK3, and/or TYK2), better selectivity over other pseudokinases (e.g., over JAK1-JH2 and/or Tyk2-JH2), and/or better ADME (absorption, distribution, metabolism, and excretion) properties including but not limited to better permeability, cytotoxicity, hepatocyte stability, solubility, and/or plasma protein binding profiles (e.g., based on assays described in the ensuing examples), than other known compounds. Selectivity over other pseudokinases can be assessed using assays analogous to JAK2 JH2 Domain Binding Assay described in the ensuing examples, utilizing other pseudokinase constructs (e.g., over JAK1-JH2 and/or Tyk2-JH2) in place of JAK2-JH2. In some embodiments, provided compounds display certain desirable characteristics in one or more assays described herein, e.g., compared to other known compounds.

In some embodiments, provided compounds are provided and/or utilized in a salt form (e.g., a pharmaceutically acceptable salt form). Reference to a compound provided herein is understood to include reference to salts thereof, unless otherwise indicated. Pharmaceutically acceptable salt forms are known in the art. For example, S. M. Berge, et al. describes pharmaceutically acceptable salts in detail in J. Pharmaceutical Sciences, 66:1-19(1977).

It will be appreciated that throughout the present disclosure, unless otherwise indicated, reference to a compound of Formula I is intended to also include II-1, II-2, II-3, III, IV-1, IV-2, IV-3, V-1, V-2, VI-1, VI-2, VII-1, VII-2, VII-3, VII-4, VII-5, VIII, and IX, and compound species of such formulas disclosed herein.

### Compositions

The present disclosure also provides compositions comprising a compound provided herein with one or more other components. In some embodiments, provided compositions comprise and/or deliver a compound described herein (e.g., compounds of Formulae I, II-1, II-2, II-3, III, IV-1, IV-2, IV-3, V-1, V-2, VI-1, VI-2, VII-1, VII-2, VII-3, VII-4, VII-5, VIII, and IX).

In some embodiments, a provided composition is a pharmaceutical composition that comprises and/or delivers a compound provided herein (e.g., compounds of Formulae I, II-1, II-2, II-3, III, IV-1, IV-2, IV-3, V-1, V-2, VI-1, VI-2, VII-1, VII-2, VII-3, VII-4, VII-5, VIII, and IX) and further comprises a pharmaceutically acceptable carrier. Pharmaceutical compositions typically contain an active agent (e.g., a compound described herein) in an amount effective to achieve a desired therapeutic effect while avoiding or minimizing adverse side effects. In some embodiments, provided pharmaceutical compositions comprise a compound described herein and one or more fillers, disintegrants, lubricants, glidants, anti-adherents, and/or anti-statics, etc. Provided pharmaceutical compositions can be in a variety of forms including oral dosage forms, topical creams, topical patches, iontophoresis forms, suppository, nasal spray and/or inhaler, eye drops, intraocular injection forms, depot forms, as well as injectable and infusible solutions. Methods of preparing pharmaceutical compositions are well known in the art.

In some embodiments, provided compounds are formulated in a unit dosage form for ease of administration and uniformity of dosage. The expression “unit dosage form” as used herein refers to a physically discrete unit of an active agent (e.g., a compound described herein) for administration to a subject. Typically, each such unit contains a predetermined quantity of active agent. In some embodiments, a unit dosage form contains an entire single dose of the agent. In some embodiments, more than one unit dosage form is administered to achieve a total single dose. In some embodiments, administration of multiple unit dosage forms is required, or expected to be required, in order to achieve an intended effect. A unit dosage form may be, for example, a liquid pharmaceutical composition containing a predetermined quantity of one or more active agents, a solid pharmaceutical composition (e.g., a tablet, a capsule, or the like) containing a predetermined amount of one or more active agents, a sustained release formulation containing a predetermined quantity of one or more active agents, or a drug delivery device containing a predetermined amount of one or more active agents, etc.

Provided compositions may be administered using any amount and any route of administration effective for treating or lessening the severity of any disease or disorder described herein.

### Uses

The present disclosure provides uses for compounds and compositions described herein. In some embodiments, provided compounds and compositions are useful in medicine (e.g., as therapy). In some embodiments, provided compounds and compositions are useful in research as, for example, analytical tools and/or control compounds in biological assays.

In some embodiments, the present disclosure provides methods of administering provided compounds or compositions to a subject in need thereof. In some embodiments, the present disclosure provides methods of administering provided compounds or compositions to a subject suffering from or susceptible to a disease, disorder, or condition associated with JAK2.

In some embodiments, provided compounds are useful as JAK2 inhibitors. In some embodiments, provided compounds are useful as JAK2 inhibitors that bind the pseudokinase (JH2) domain of JAK2. In some embodiments, the present disclosure provides methods of inhibiting JAK2 in a subject comprising administering a provided compound or composition. In some embodiments, the present disclosure provides methods of inhibiting JAK2 in a biological sample comprising contacting the sample with a provided compound or composition.

In some embodiments, the present disclosure provides methods of treating a disease, disorder, or condition associated with JAK2. JAK (e.g., JAK2) has been implicated in various diseases, disorders, and conditions, such as myeloproliferative neoplasms (Vainchenker, W. et al., F1000Research 2018, 7(F1000 Faculty Rev):82), atopic dermatitis (Rodrigues, M. A. and Torres, T. J. Derm. Treat. 2019, 31(1), 33-40) and acute respiratory syndrome, hyperinflammation, and/or cytokine storm syndrome (The Lancet. doi:10.1016/S0140-6736(20)30628-0). Accordingly, in some embodiments, the present disclosure provides methods of treating a disease, disorder or condition associated with JAK2 in a subject in need thereof comprising administering to the subject a provided compound or composition. In some embodiments, a disease, disorder, or condition is associated with overexpression of JAK2.

In some embodiments, the present disclosure provides a method of treating a disease, disorder, or condition associated with JAK2, wherein the disease, disorder, or condition is a cancer, a proliferative disease, a hematological malignancy, or an inflammatory disease, disorder, or condition.

In some embodiments, the present disclosure provides methods of treating cancer, comprising administering a provided compound or composition to a subject in need thereof. In some embodiments, the present disclosure provides methods of treating proliferative diseases, comprising administering a provided compound or composition to a subject in need thereof.

In some embodiments, the present disclosure provides methods of treating a hematological malignancy, comprising administering a provided compound or composition to a subject in need thereof. In some embodiments, a hematological malignancy is leukemia (e.g., chronic lymphocytic leukemia, acute lymphoblastic leukemia, T-cell acute lymphoblastic leukemia, chronic myelogenous leukemia, acute myelogenous leukemia, or acute monocytic leukemia). In some embodiments, a hematological malignancy is lymphoma (e.g., Burkitt's lymphoma, Hodgkin's lymphoma, or non-Hodgkin's lymphoma). In some embodiments, a non-Hodgkin's lymphoma is a B-cell lymphoma. In some embodiments, a non-Hodgkin's lymphoma is a NK/T-cell lymphoma (e.g., cutaneous T-cell lymphoma). In some embodiments, a hematological malignancy is myeloma (e.g., multiple myeloma). In some embodiments, a hematological malignancy is myeloproliferative neoplasm (e.g., polycythemia vera, essential thrombocytopenia, or myelofibrosis). In some embodiments, a hematological malignancy is myelodysplastic syndrome.

In some embodiments, the present disclosure provides methods of treating an inflammatory disease, disorder, or condition (e.g., acute respiratory syndrome, hyperinflammation, and/or cytokine storm syndrome (including those associated with COVID-19) or atopic dermatitis), comprising administering a provided compound or composition to a subject in need thereof.

In some embodiments, a provided compound or composition is administered as part of a combination therapy. As used herein, the term “combination therapy” refers to those situations in which a subject is simultaneously exposed to two or more therapeutic or prophylactic regimens (e.g., two or more therapeutic or prophylactic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

For example, in some embodiments, a provided compound or composition is administered to a subject who is receiving or has received one or more additional therapies (e.g., an anti-cancer therapy and/or therapy to address one or more side effects of such anti-cancer therapy, or otherwise to provide palliative care). Exemplary additional therapies include BCL2 inhibitors (e.g., venetoclax), HDAC inhibitors (e.g., vorinostat), BET inhibitors (e.g., mivebresib), proteasome inhibitors (e.g., bortezomib), LSD1 inhibitors (e.g., IMG-7289), and CXCR2 inhibitors. Useful combinations of a JAK2 inhibitor with BCL2, HDAC, BET, and proteasome inhibitors have been demonstrated in cells derived from cutaneous T-cell lymphoma patients (Yumeen, S., et al., Blood Adv. 2020, 4(10), 2213-2226). A combination of a JAK2 inhibitor with a LSD1 inhibitor demonstrated good efficacy in a mouse model of myeloproliferative neoplasms (Jutzi, J. S., et al., HemaSphere 2018, 2(3), http://dx.doi.org/10.1097/HS9.0000000000000054). CXCR2 activity has been shown to modulate signaling pathways involved in tumor growth, angiogenesis, and/or metastasis, including the JAK-STAT3 pathway (Jaffer, T., Ma, D. Transl. Cancer Res. 2016, 5(Suppl. 4), 5616-5628).

### EXEMPLARY EMBODIMENTS

The following numbered embodiments, while non-limiting, are exemplary of certain aspects of the present disclosure:

1. A compound represented by formula I:

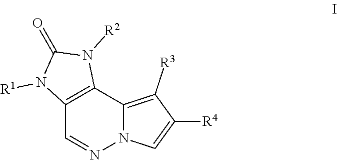

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is optionally substituted with one or more instances of -L-R⁵;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a}; or
R² and R³ are connected by *-L^{a}-R⁶-L^{b}-**, where * represents a point of attachment to R², and ** represents a point of attachment to R³;
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
L^{b} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
R⁶ is optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, or optionally substituted C₆-C₁₀ aryl;
R⁴ is C₁-C₆ aliphatic, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

2. The compound of embodiment 1, wherein R¹ is optionally substituted C₁-C₆ aliphatic.

3. The compound of embodiment 1 or 2, wherein R¹ is —CH₃ or -CD₃.

4. The compound of any one of embodiments 1-3, wherein R² is C₁-C₆ aliphatic, C₃-C₁₂ cycloaliphatic, or 4- to 10-membered heterocycle comprising 1 to 4 heteroatoms selected from N, O, and S, wherein R² is optionally substituted with one or more instances of -L-R⁵.

5. The compound of embodiment 4, wherein R² is C₃-C₁₂ cycloaliphatic, wherein R² is optionally substituted with -L-R⁵.

6. The compound of embodiment 4, wherein R² is monocyclic C₃-C₆ cycloaliphatic, wherein R² is optionally substituted with -L-R⁵.

7. The compound of embodiment 4, wherein R² is 4- to 10-membered heterocycle comprising 1 to 4 heteroatoms selected from N, O, and S, wherein R² is optionally substituted with -L-R⁵.

8. The compound of any one of embodiments 1-3, wherein R² is:

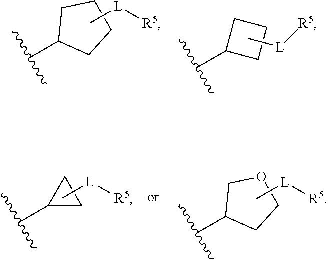

9. The compound of any one of embodiments 1-3, wherein R² is:

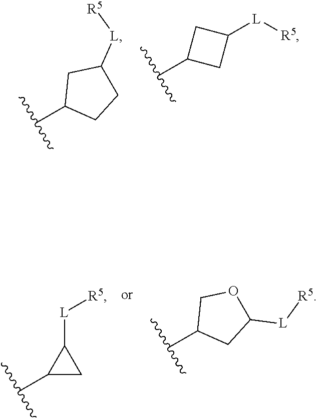

10. The compound of any one of embodiments 1-9, wherein L is selected from —N(R^{a})C(O)O—, —C(O)N(R^{a})—, and —C(O)—.

11. The compound of any one of embodiments 1-9, wherein L is selected from —N(H)C(O)O—, —C(O)NH—, —C(O)NCH₂—, and —C(O)—.

12. The compound of any one of embodiments 1-11, wherein R⁵ is selected from optionally substituted C₁-C₆ aliphatic, optionally substituted 2- to 10-membered heteroaliphatic, and optionally substituted 4- to 10-membered heterocycle comprising 1 to 4 heteroatoms selected from N, O, and S, and optionally substituted 5- to 10-membered heteroaryl comprising 1 to 4 heteroatoms selected from N, O, and S.

13. The compound of any one of embodiments 1-9, wherein L is selected from —N(R^{a})C(O)O— and —C(O)N(R^{a})—, and wherein R⁵ is optionally substituted C₁-C₆ aliphatic.

14. The compound of any one of embodiments 1-9, wherein -L-R⁵ is selected from:

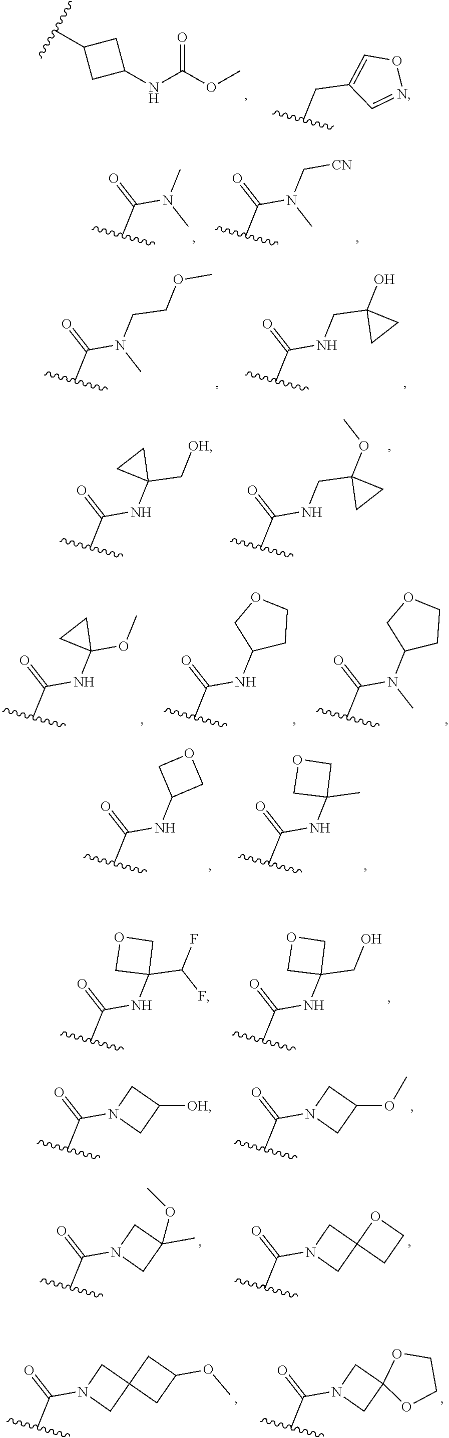

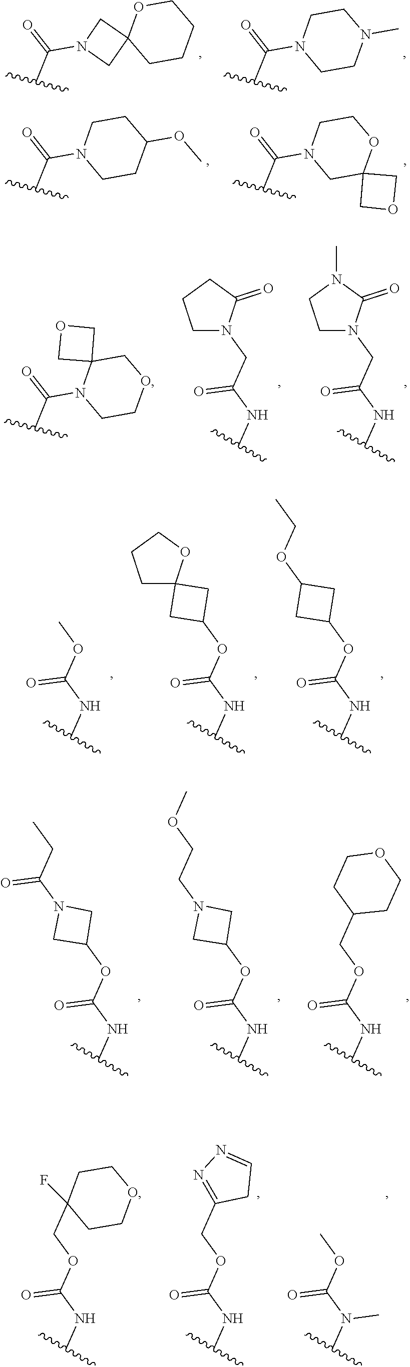

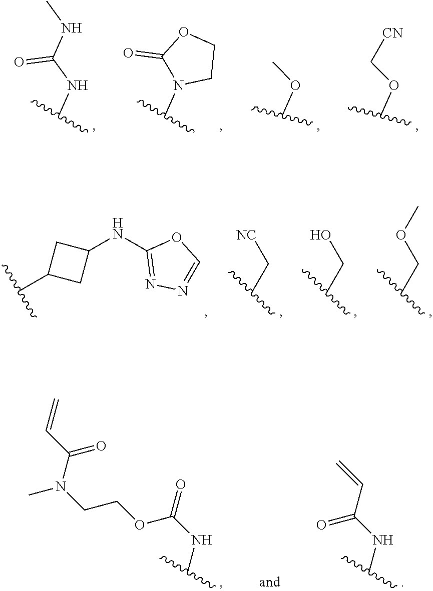

15. The compound of any one of embodiments 1-9, wherein R² is:

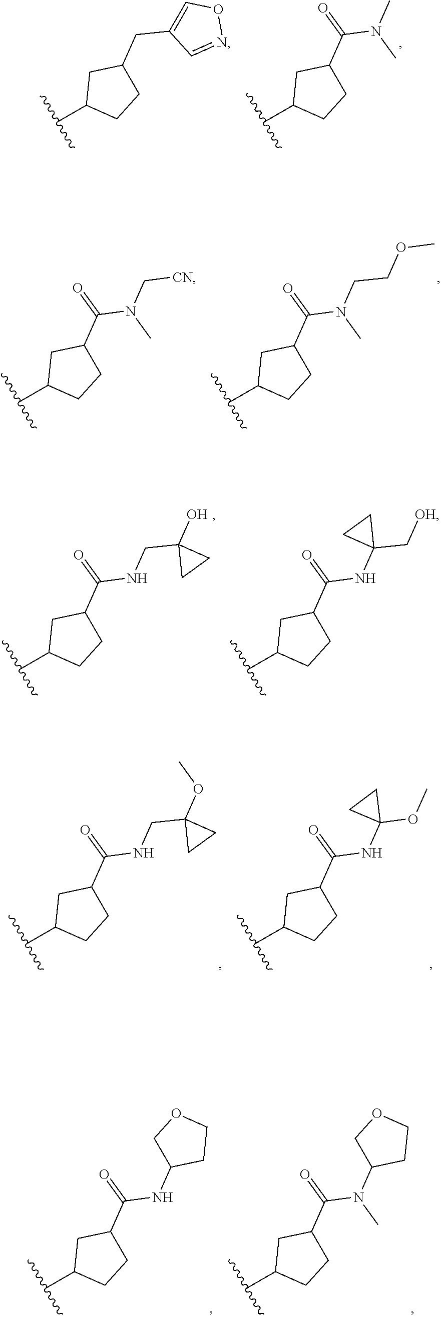

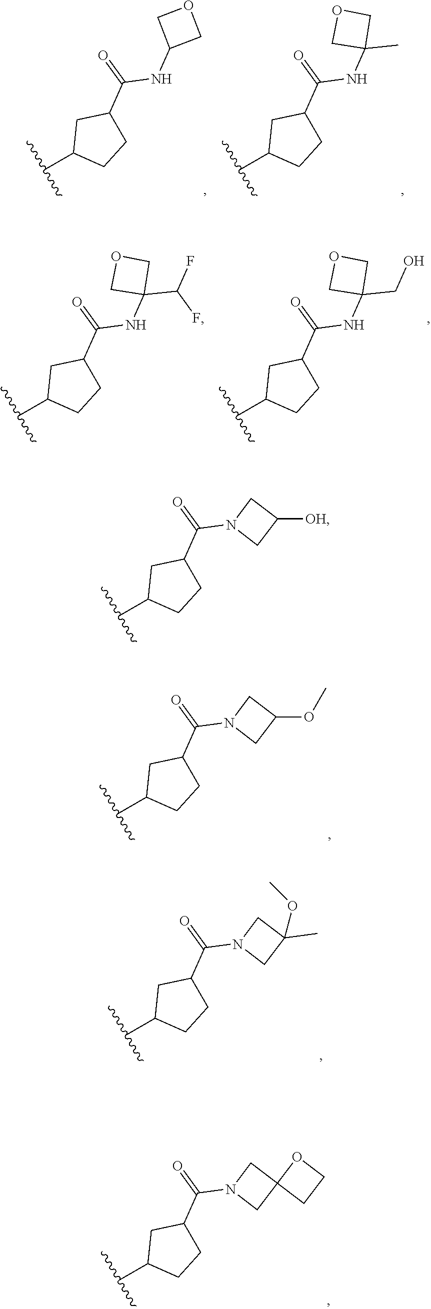

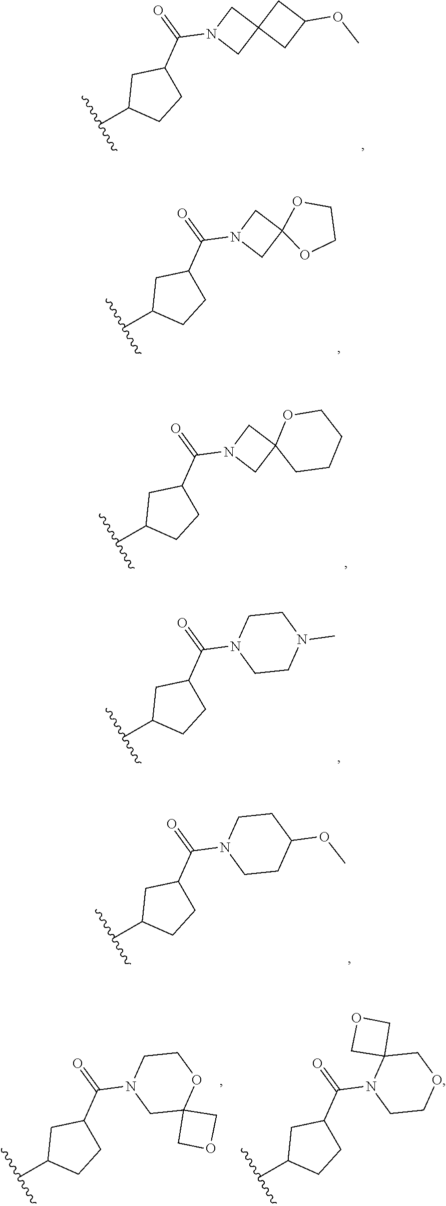

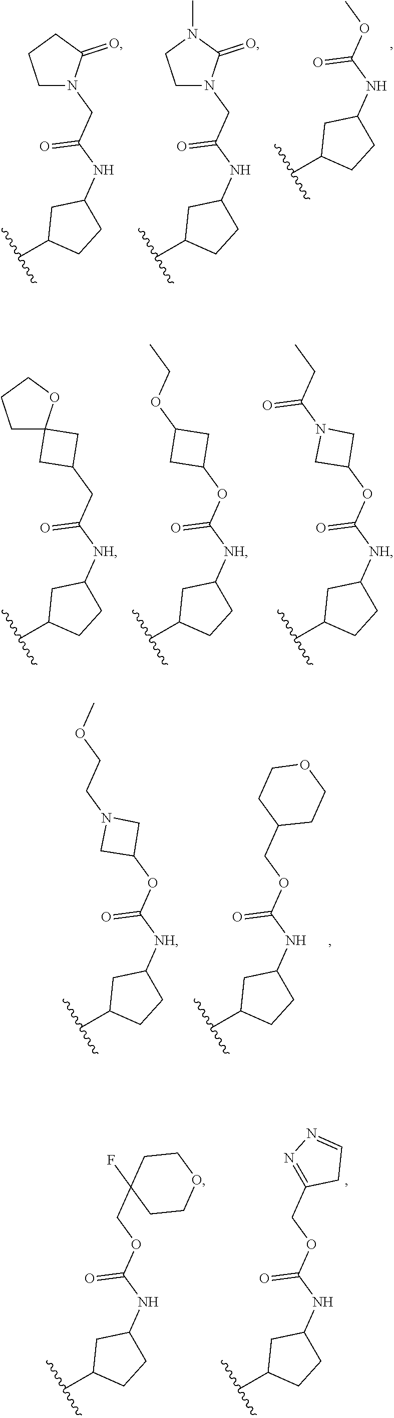

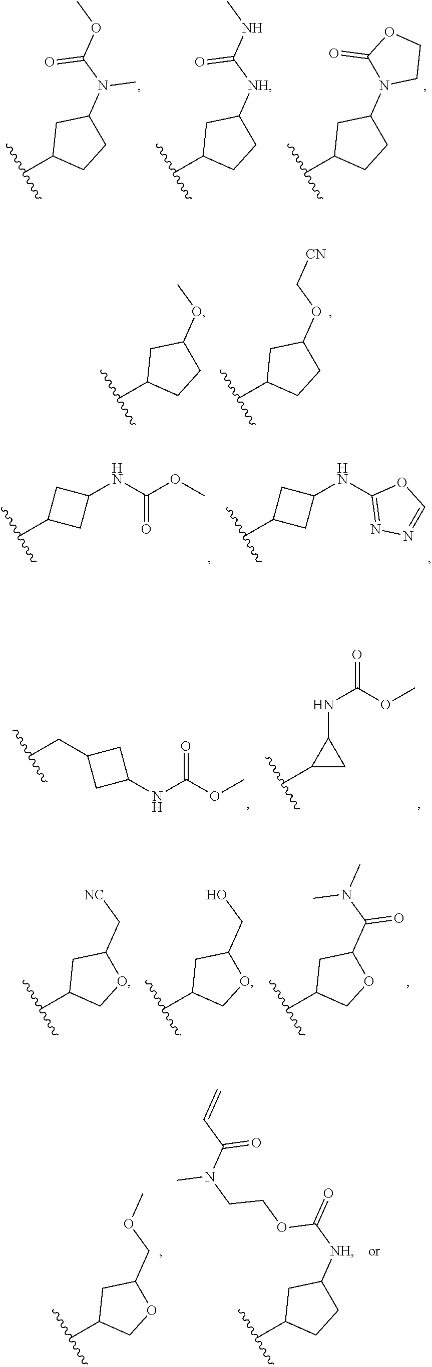

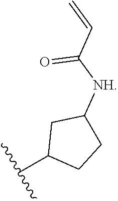 [SMILES: *C1CCC(NC(=O)C=C)C1]

16. The compound of any one of embodiments 1-15, wherein R³ is 5- to 12-membered heteroaryl comprising 1 to 4 heteroatoms selected from N, O, and S, and R³ is optionally substituted with one or more instances of R^{3a}.

17. The compound of any one of embodiments 1-15, wherein R³ is bicyclic 7- to 12-membered heteroaryl comprising 1 to 4 heteroatoms selected from N, O, and S, and R³ is optionally substituted with one or more instances of R^{3a}.

18. The compound of any one of embodiments 1-15, wherein R³ is:

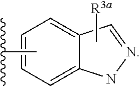

19. The compound of any one of embodiments 1-15, wherein R³ is C₆-C₁₂ aryl, and R³ is optionally substituted with one or more instances of R^{3a}.

20. The compound of any one of embodiments 1-15, wherein R³ is:

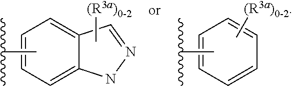

21. The compound of any one of embodiments 1-15, wherein R³ is

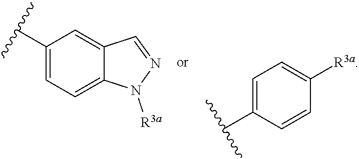

22. The compound of any one of embodiments 1-21, wherein each R^{3a} is independently selected from optionally substituted C₁-C₆ aliphatic, optionally substituted 2- to 10-membered heteroaliphatic, optionally substituted C₃-C₁₂ cycloaliphatic, and optionally substituted 4- to 10-membered heterocycle comprising 1 to 4 heteroatoms selected from N, O, and S.

23. The compound any one of embodiments 1-21, wherein R^{3a} is —CH₃, —CD₃,

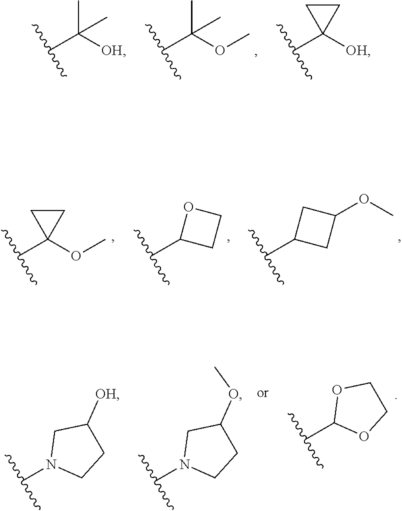

24. The compound of any one of embodiments 1-15, wherein R³ is

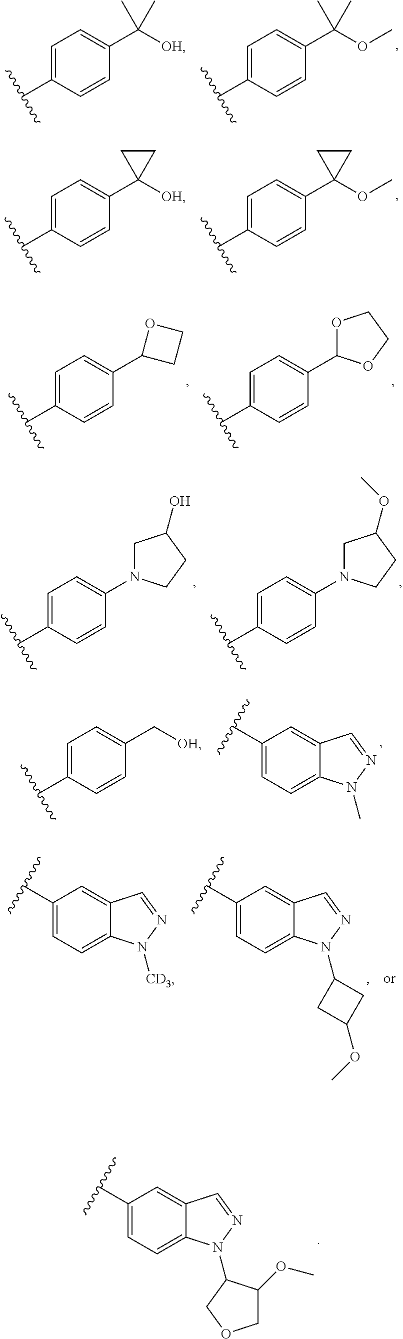

25. The compound of any one of embodiments 1-15, wherein R² and R³ are connected by *-L^{a}-R⁶-L^{b}-**, where * represents a point of attachment to R², and ** represents a point of attachment to R³.

26. The compound of embodiment 25, wherein L^{a} is a bond, —N(R^{a})C(O)O—, —OC(O)N(R^{a}), —C(O)N(R^{a})—, —N(R^{a})C(O)—, —N(R^{a})—, —O—, —C(O)—, or —C(O)O—.

27. The compound of embodiment 25 or 26, wherein L^{a} is a bond, —N(H)C(O)O—, —OC(O)N(H)—, —C(O)N(H)—, or —N(H)C(O)—.

28. The compound of any one of embodiments 25-27, wherein L^{b} is a bond, —N(R^{a}), —O—, —C(O)—, —C(O)O—, or —OC(O)—.

29. The compound of any one of embodiments 25-28, wherein L^{a} is —N(R^{a})C(O)O— or —C(O)N(R^{a})—, and L^{b} is a bond.

30. The compound of any one of embodiments 25-29, wherein R⁶ is optionally substituted C₁-C₆aliphatic, optionally substituted 2- to 10-membered heteroaliphatic, optionally substituted C₃-C₁₂ cycloaliphatic, or optionally substituted 4- to 10-membered heterocycle comprising 1 to 4 heteroatoms selected from N, O, and S.

31. The compound of any one of embodiments 25-29, wherein R⁶ is optionally substituted C₁-C₆ aliphatic or optionally substituted 2- to 10-membered heteroaliphatic.

32. The compound of any one of embodiments 25-29, wherein R⁶ is optionally substituted C₁-C₆aliphatic.

33. The compound of any one of embodiments 25-30, wherein moiety:

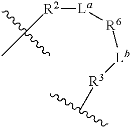

is

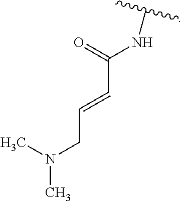 [SMILES: *NC(=O)/C=C/CN(C)C]

34. The compound of any one of embodiments 25-33, wherein moiety:

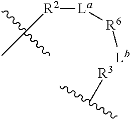

is

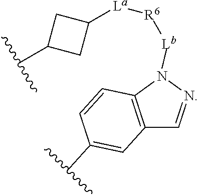

35. The compound of embodiment 34, wherein moiety:

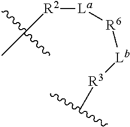

is

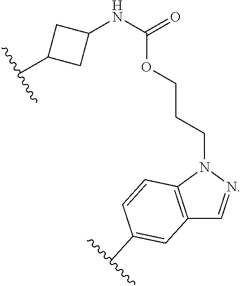 [SMILES: *c1ccc2c(cnn2CCCOC(=O)NC2CC(*)C2)c1]

36. The compound of any one of embodiments 1-35, wherein R⁴ is 5- to 10-membered heteroaryl comprising 1 to 4 heteroatoms selected from N, O, and S, or 4- to 10-membered heterocycle comprising 1 to 4 heteroatoms selected from N, O, and S, and wherein R⁴ is optionally substituted with one or more instances of R^{4a}.

37. The compound of any one of embodiments 1-35, wherein R⁴ is 5- to 10-membered heteroaryl comprising 1 to 4 heteroatoms selected from N, O, and S, and wherein R⁴ is optionally substituted with one or more instances of R^{4a}.

38. The compound of any one of embodiments 1-35, wherein R⁴ is 7- to 10-membered bicyclic heteroaryl comprising 1 to 4 heteroatoms selected from N, O, and S, and wherein R⁴ is optionally substituted with one or more instances of R^{4a}.

39. The compound of any one of embodiments 1-35, wherein R⁴ is 5- to 6-membered monocyclic heteroaryl comprising 1 to 3 heteroatoms selected from N, O, and S, and wherein R⁴ is optionally substituted with one or more instance of R^{4a}.

40. The compound of any one of embodiments 1-35, wherein R⁴ is 5- to 6-membered monocyclic heteroaryl comprising 1 to 3 heteroatoms selected from N, O, and S, and wherein R⁴ is substituted with one or more instances of R^{4a}, wherein R^{4a} is selected from optionally substituted C₁-C₆ aliphatic and halogen.

41. The compound of any one of embodiments 1-35, wherein R⁴ is 4- to 10-membered heterocycle comprising 1 to 4 heteroatoms selected from N, O, and S, and wherein R⁴ is optionally substituted with one or more instances of R^{4a}.

42. The compound of any one of embodiments 1-41, wherein each instance of R^{4a} is selected from optionally substituted C₁-C₆ aliphatic and halogen.

43. The compound of any one of embodiments 1-35, wherein R⁴ is selected from:

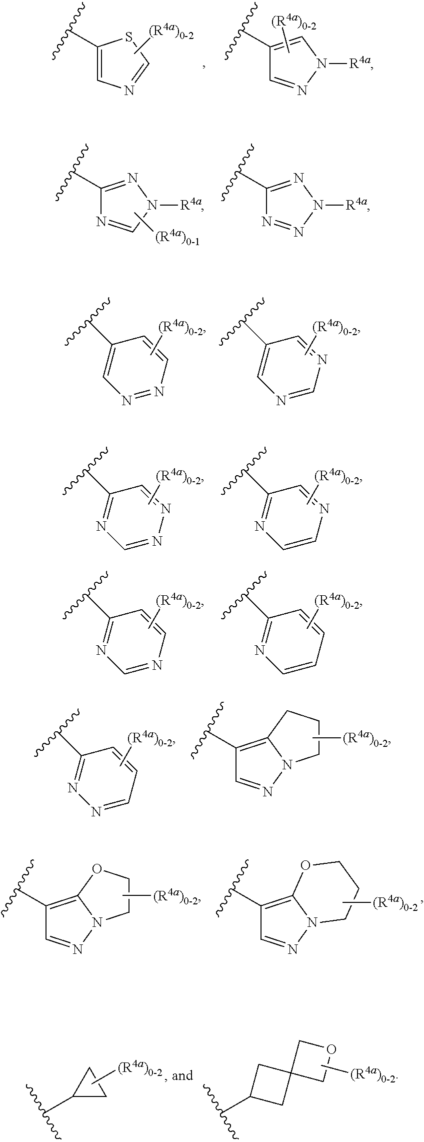

44. The compound of any one of embodiments 1-35, wherein R⁴ is selected from:

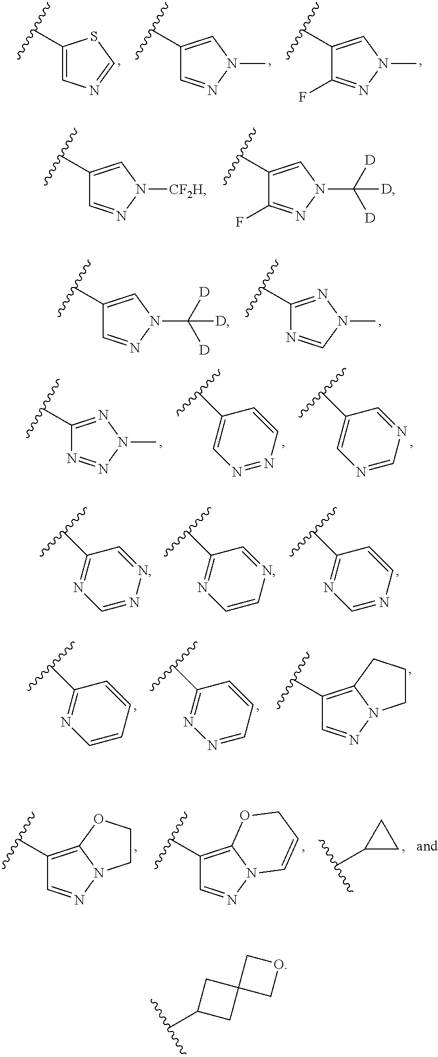

45. The compound of embodiment 1, wherein R² is C₃-C₁₂ cycloaliphatic or 4- to 10-membered heterocycle comprising 1 to 4 heteroatoms selected from N, O, and S; and wherein R² is substituted with one instance of -L-R⁵.

46. The compound of any one of embodiments 1-45, wherein the compound is represented by formula II-1 or II-2:

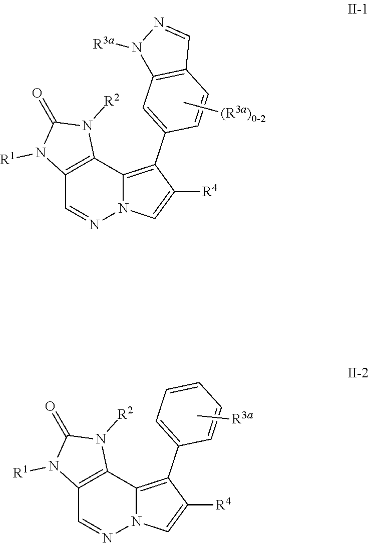

or a pharmaceutically acceptable salt thereof.

47. The compound of any one of embodiments 1-46, wherein the compound is represented by formula III:

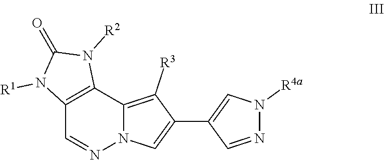

or a pharmaceutically acceptable salt thereof.

48. The compound of any one of embodiments 1-47, wherein the compound is represented by formula IV-1 or IV-2:

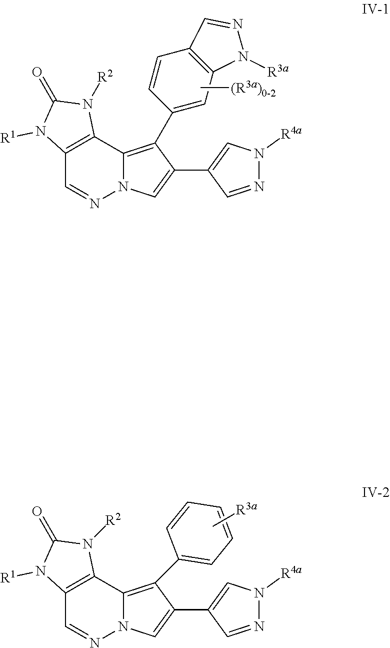

or a pharmaceutically acceptable salt thereof.

49. The compound of any one of embodiments 1-48, wherein the compound is represented by formula V:

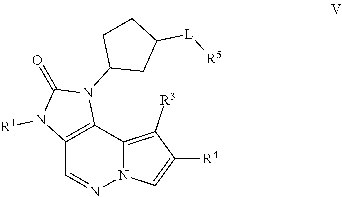

or a pharmaceutically acceptable salt thereof.

50. The compound of any one of embodiments 1-49, wherein the compound is represented by formula VI:

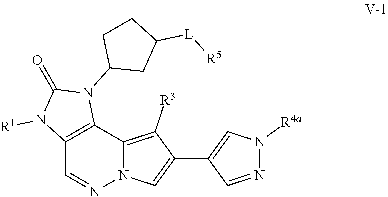

or a pharmaceutically acceptable salt thereof.

51. The compound of any one of embodiments 1-50, wherein the compound is represented by formula VII-1 or VII-2:

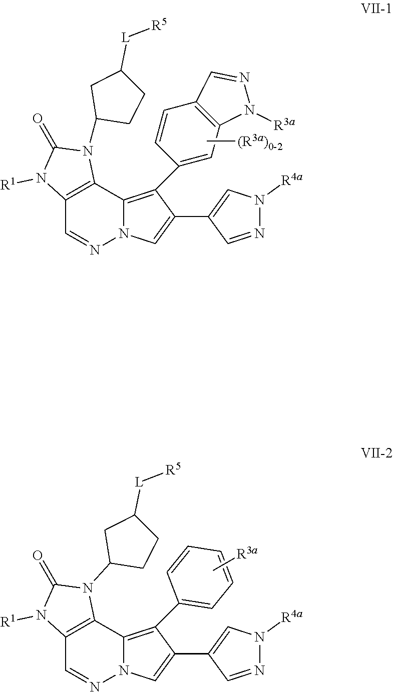

52. The compound of any one of embodiments 1-51, wherein the compound is represented by formula VIII:

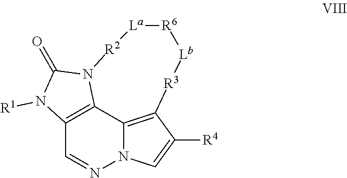

or a pharmaceutically acceptable salt thereof.

53. The compound of embodiment 1, wherein the compound is selected from Table 1C, or a pharmaceutically acceptable salt thereof.

54. A pharmaceutical composition comprising a compound of any one of embodiments 1-53, or a pharmaceutically acceptable salt thereof, and a pharmaceutically acceptable carrier.

55. A method of inhibiting JAK2 in a subject, comprising administering to the subject the compound of any one of embodiments 1-53 or the pharmaceutical composition of embodiment 54.

56. A method of treating a disease, disorder, or condition associated with JAK2, comprising administering to a subject in need thereof the compound of any one of embodiments 1-53 or the pharmaceutical composition of embodiment 54.

57. A method of treating cancer, comprising administering to a subject in need thereof the compound of any one of embodiments 1-53 or the pharmaceutical composition of embodiment 54.

58. A method of treating a hematological malignancy, comprising administering to a subject in need thereof the compound of any one of embodiments 1-53 or the pharmaceutical composition of embodiment 54.

59. The method of embodiment 58, wherein the hematological malignancy is leukemia or lymphoma.

60. A method of treating a myeloproliferative neoplasm, comprising administering to a subject in need thereof the compound of any one of embodiments 1-53 or the pharmaceutical composition of embodiment 54.

61. The method of embodiment 60, wherein the myeloproliferative neoplasm is polycythemia vera, essential thrombocytopenia, or myelofibrosis.

62. A compound of formula I:

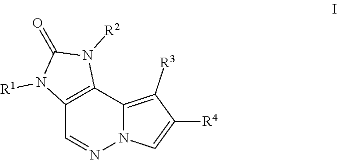

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is optionally substituted with one or more instances of -L-R⁵ or R⁷;
each L is independently selected from the group consisting of a bond, —N(R^{a})C(O)N(R^{a}), —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
each R⁷ is independently selected from the group consisting of halogen, —OH, —SH, and —NH₂;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a}; or
R² and R³ are connected by *-L^{a}-R⁶-L^{b}-**, wherein * represents a point of attachment to R², and ** represents a point of attachment to R³;
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
L^{b} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
R⁶ is optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, or optionally substituted C₆-C₁₀ aryl;
R⁴ is C₁-C₆ aliphatic, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

63. A compound of formula I:

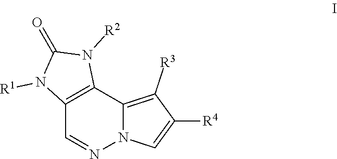

or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is optionally substituted with one or more instances of -L-R⁵ or R⁷;
each L is independently selected from the group consisting of a bond, —N(R^{a})C(O)N(R^{a}), —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
each R⁷ is independently selected from the group consisting of halogen, —OH, —SH, and —NH₂;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a};
R⁴ is C₁-C₆ aliphatic, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

64. The compound of embodiment 62 or 63, wherein R¹ is optionally substituted C₁-C₆ aliphatic.

65. The compound of any one of embodiments 62-64, wherein R¹ is —CH₃ or -CD₃.

66. The compound of any one of embodiments 62-65, wherein R² is C₃-C₁₂ cycloaliphatic or 4- to 10-membered heterocycle comprising 1 to 4 heteroatoms selected from N, O, and S; and wherein R² is substituted with one instance of -L-R⁵.

67. The compound of any one of embodiments 62-66, wherein R² is C₃-C₇ monocyclic cycloaliphatic or 4- to 6-membered monocyclic heterocycle comprising 1 to 2 heteroatoms selected from N, O, and S; and wherein R² is substituted with 0-2 instances of -L-R⁵ or R⁷.

68. The compound of any one of embodiments 62-67, wherein R² is substituted with 1-2 instances of -L-R⁵ or R⁷.

69. The compound of embodiment 67 or 68, wherein R² is C₃-C₆ monocyclic cycloaliphatic, and wherein R² is substituted with 1-2 instances of -L-R⁵ or R⁷.

70. The compound of embodiment 67 or 68, wherein R² is 4- to 6-membered heterocycle comprising 1 to 2 heteroatoms selected from N, O, and S, and wherein R² is substituted with 1-2 instances of -L-R⁵ or R⁷.

71. The compound of any one of embodiments 62-65, wherein R² is:

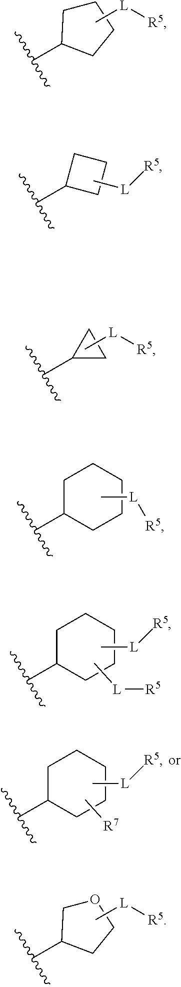

72. The compound of any one of embodiments 62-65, wherein R² is:

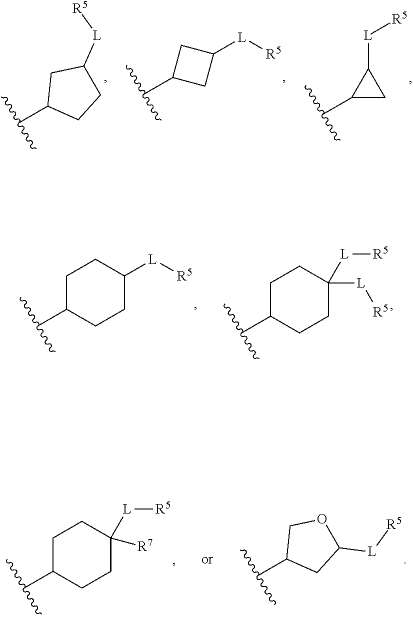

73. The compound of any one of embodiments 62-72, wherein each L is independently selected from a bond, —N(R^{a})C(O)O—, —OC(O)N(R^{a})—, —C(O)N(R^{a})—, —N(R^{a})C(O)—, —O—, and —C(O)—.

74. The compound of embodiment 73, wherein each L is independently selected from a bond, —N(R^{a})C(O)O—, and —N(R^{a})C(O)—.

75. The compound of any one of embodiments 62-74, wherein each R⁵ is independently selected from optionally substituted C₁-C₆ aliphatic, optionally substituted C₃-C₆ cycloaliphatic, optionally substituted 4- to 6-membered heterocycle comprising 1 to 2 heteroatoms selected from N, O, and S, and optionally substituted 5- to 6-membered heteroaryl comprising 1 to 4 heteroatoms selected from N, O, and S.

76. The compound of any one of embodiments 62-72, wherein each -L-R⁵ is independently selected from: —CH₃, —CH₂OH, —CH₂OCH₃,

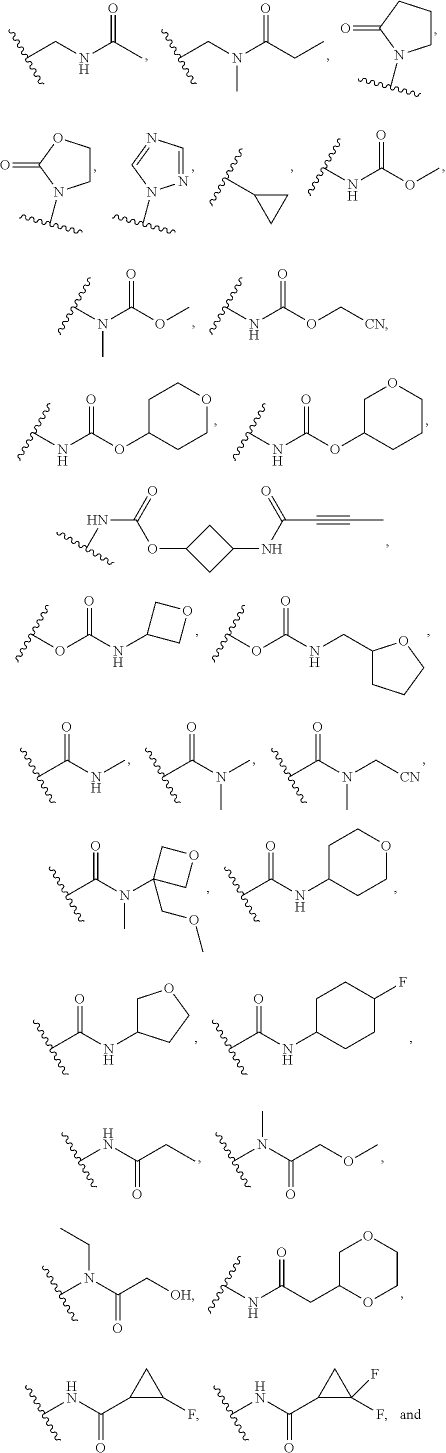

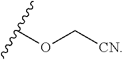 [SMILES: *OCC#N]

77. The compound of any one of embodiments 62-76, wherein each R⁷ is independently selected from the group consisting of —OH, —SH, and —NH₂.

78. The compound of any one of embodiments 62-65, wherein R² optionally substituted with one or more -L-R⁵ or R⁷ is selected from:

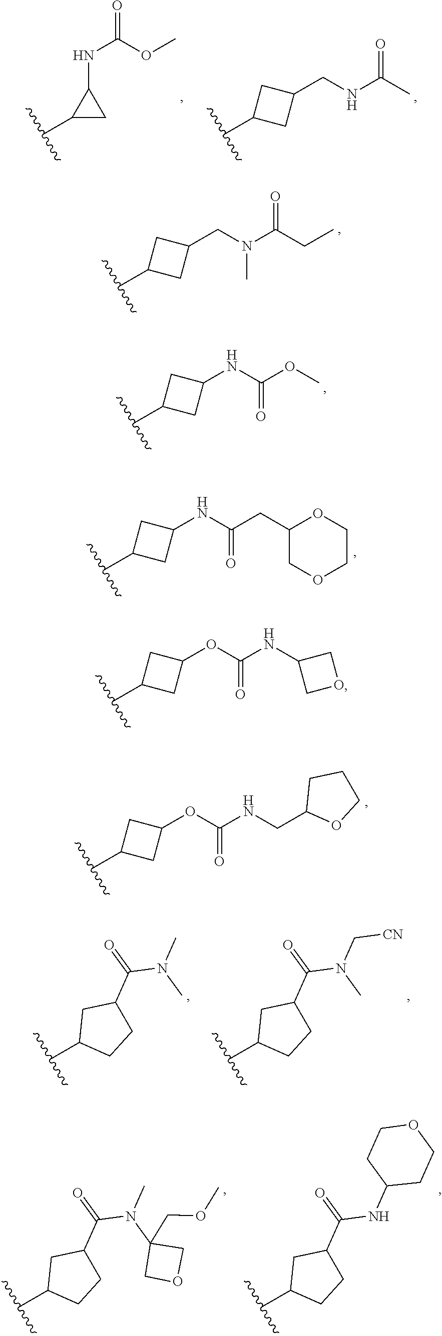

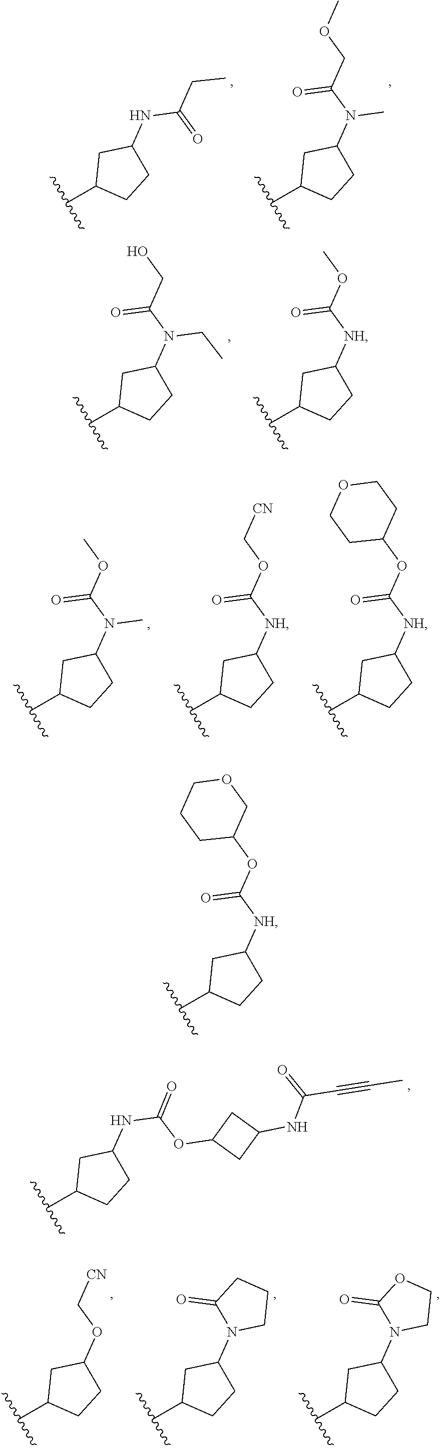

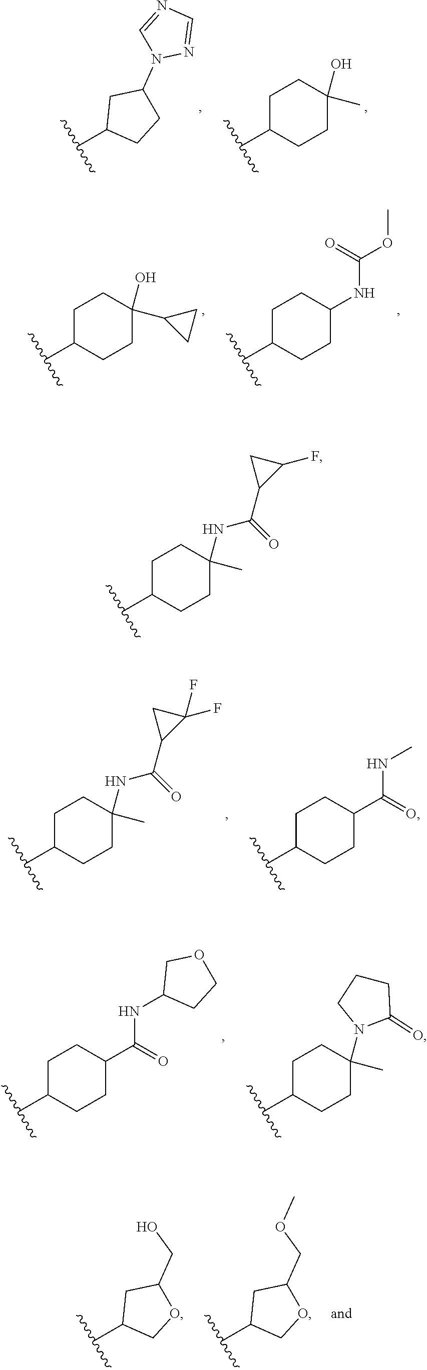

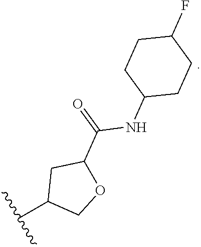 [SMILES: *C1COC(C(=O)NC2CCC(F)CC2)C1]

79. The compound of any one of embodiments 62-78, wherein R³ is 5- to 10-membered heteroaryl comprising 1 to 4 heteroatoms selected from N, O, and S, 9- to 10-membered heterocycle comprising 1 to 4 heteroatoms selected from N, O, and S, or phenyl, wherein R³ is optionally substituted with 0-3 instances of R^{3a}.

80. The compound of any one of embodiments 62-78, wherein R³ is monocyclic 5- to 6-membered heteroaryl comprising 1 to 3 heteroatoms selected from N, O, and S, and wherein R³ is optionally substituted with 0-3 instances of R^{3a}.

81. The compound of any one of embodiments 62-78, wherein R³ is bicyclic 8- to 10-membered heteroaryl comprising 1 to 4 heteroatoms selected from N, O, and S, and wherein R³ is optionally substituted with 0-3 instances of R^{3a}.

82. The compound of embodiment 81, wherein R³ is:

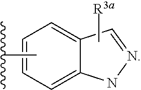

83. The compound of any one of embodiments 62-78, wherein R³ is 9- to 10-membered heterocycle comprising 1 to 4 heteroatoms selected from N, O, and S, and wherein R³ is optionally substituted with 0-3 instances of R^{3a}.

84. The compound of any one of embodiments 62-78, wherein R³ is phenyl, and R³ is optionally substituted with 0-3 instances of R^{3a}.

85. The compound of any one of embodiments 62-84, wherein each R^{3a} is independently selected from optionally substituted C₁-C₆ aliphatic, optionally substituted C₃-C₆ cycloaliphatic, halogen, —C(O)N(R^{a})₂, and —OR^{a}.

86. The compound of any one of embodiments 62-78, wherein R³ optionally substituted with one or more instances of R^{3a} is:

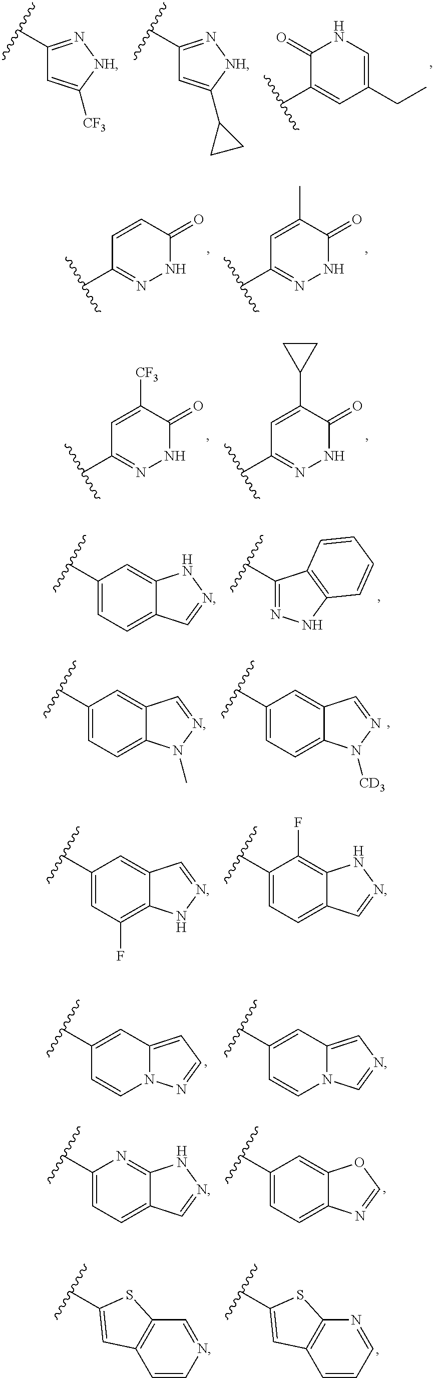

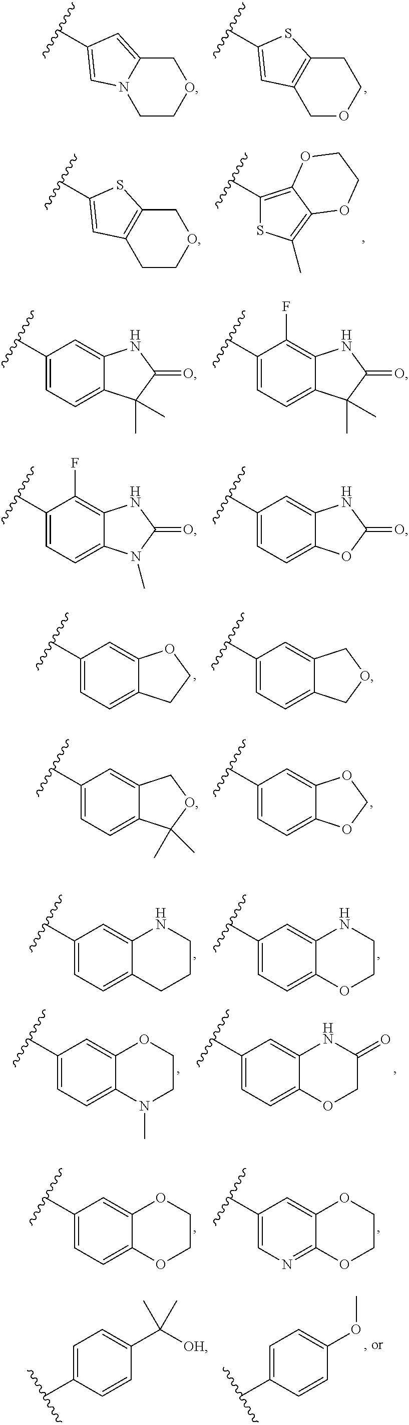

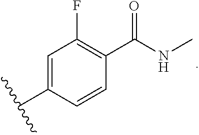 [SMILES: *c1ccc(C(=O)NC)c(F)c1]

87. The compound of any one of embodiments 62 or 64-86, wherein R² and R³ are connected by *-L^{a}-R⁶-L^{b}-**, wherein * represents a point of attachment to R², and ** represents a point of attachment to R³.

88. The compound of embodiment 87, wherein L^{a} is a bond, —N(R^{a})C(O)O—, —OC(O)N(R^{a})—, —C(O)N(R^{a})—, —N(R^{a})C(O)—, —N(R^{a})—, —O—, —C(O)—, or —C(O)O—.

89. The compound of embodiment 87 or 88, wherein L^{b} is a bond, —N(R^{a})—, —O—, —C(O)—, —C(O)O—, or —OC(O)—.

90. The compound of any one of embodiments 87-89, wherein R⁶ is optionally substituted C₁-C₆aliphatic, optionally substituted 2- to 10-membered heteroaliphatic, optionally substituted C₃-C₁₂ cycloaliphatic, or optionally substituted 4- to 10-membered heterocycle comprising 1 to 4 heteroatoms selected from N, O, and S.

91. The compound of embodiment 90, wherein R⁶ is optionally substituted C₁-C₆ aliphatic.

92. The compound of any one of embodiments 87-91, wherein a moiety:

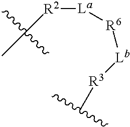

is

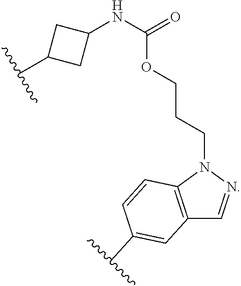 [SMILES: *c1ccc2c(cnn2CCCOC(=O)NC2CC(*)C2)c1]

93. The compound of any one of embodiments 62-92, wherein R⁴ is 5- to 10-membered heteroaryl comprising 1 to 4 heteroatoms selected from N, O, and S or 4- to 10-membered heterocycle comprising 1 to 4 heteroatoms selected from N, O, and S, and wherein R⁴ is optionally substituted with 0-3 instances of R^{4a}.

94. The compound of any one of embodiments 62-92, wherein R⁴ is monocyclic 5- to 6-membered heteroaryl comprising 1 to 3 heteroatoms selected from N, O, and S, and is optionally substituted with 0-3 instances of R^{4a}.

95. The compound of any one of embodiments 62-92, wherein R⁴ is 8- to 10-membered bicyclic heteroaryl having 1 to 4 heteroatoms selected from N, O, and S and is optionally substituted with 0-3 instances of R^{4a}.

96. The compound of any one of embodiments 62-92, wherein R⁴ is 4- to 6-membered monocyclic heterocycle comprising 1 to 3 heteroatoms selected from N, O, and S and is optionally substituted with 0-3 instances of R^{4a}.

97. The compound of any one of embodiments 62-96, wherein each R^{4a} is independently selected from optionally substituted C₁-C₆ aliphatic, —OR^{a}, and halogen.

98. The compound of any one of embodiments 62-92, wherein R⁴ optionally substituted with one or more instances R^{4a} is selected from:

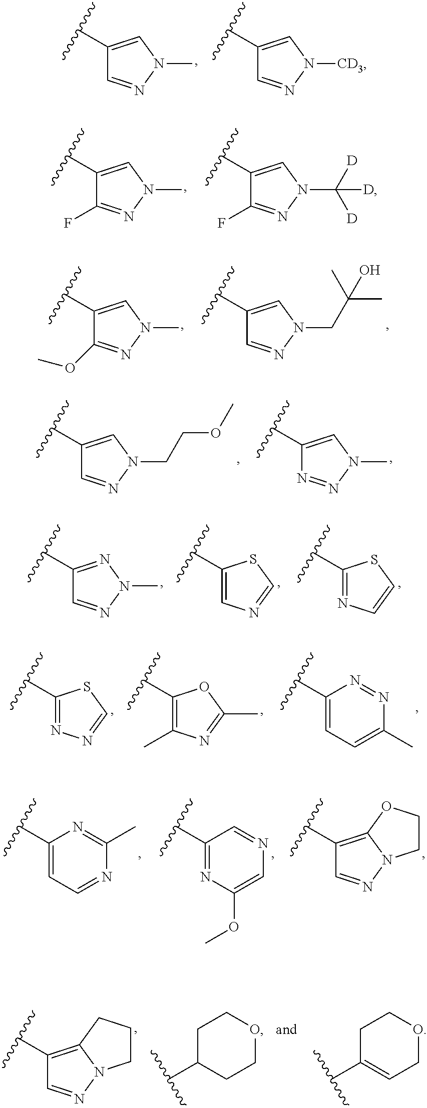

99. The compound of any one of embodiments 62-98, wherein the compound is represented by formula II-1, II-2, or II-3:

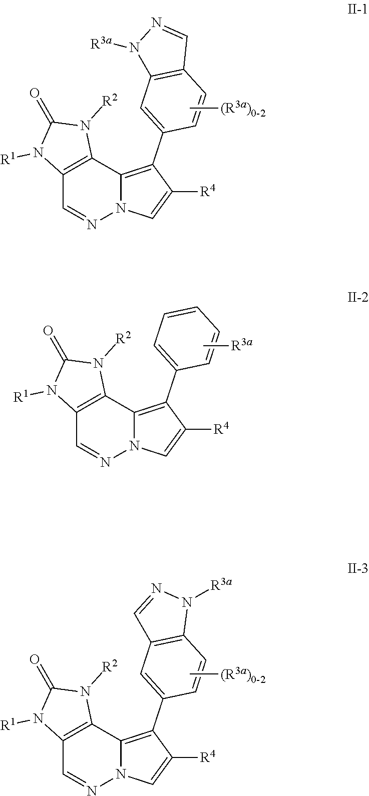

or a pharmaceutically acceptable salt thereof.

100. The compound of any one of embodiments 62-98, wherein the compound is represented by formula III:

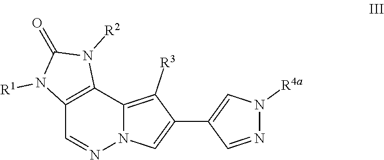

or a pharmaceutically acceptable salt thereof.

101. The compound of any one of embodiments 62-98, wherein the compound is represented by formula IV-1, IV-2, or IV-3:

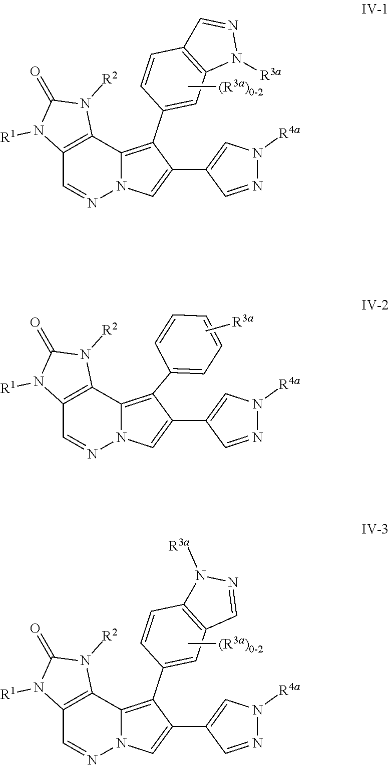

or a pharmaceutically acceptable salt thereof.

102. The compound of any one of embodiments 62-98, wherein the compound is represented by formula V-1 or V-2:

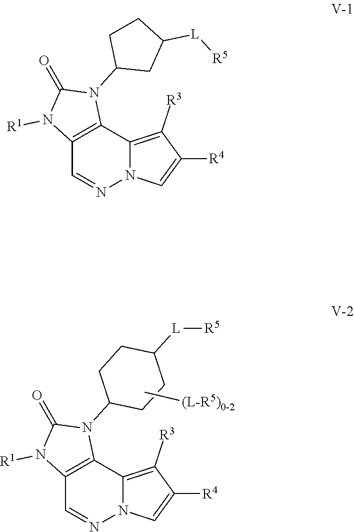

or a pharmaceutically acceptable salt thereof.

103. The compound of any one of embodiments 62-98, wherein the compound is represented by formula VI:

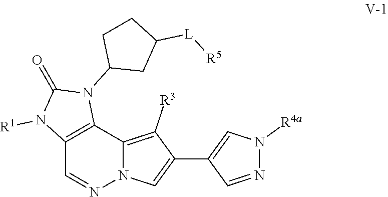

or a pharmaceutically acceptable salt thereof.

104. The compound of any one of embodiments 62-98, wherein the compound is represented by formula VII-1, VII-2, VII-3, VII-4, or VII-5:

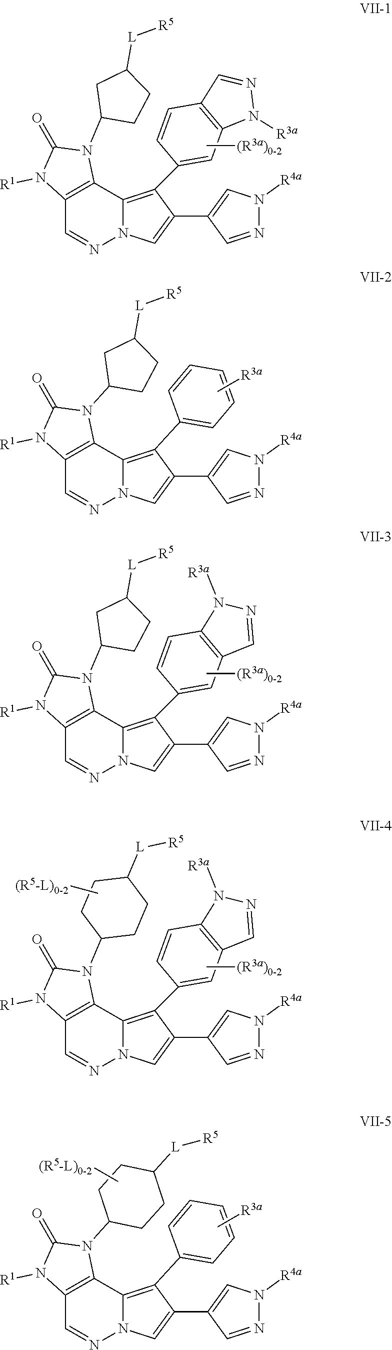

or a pharmaceutically acceptable salt thereof.

105. The compound of any one of embodiments 62 or 64-98, wherein the compound is represented by formula VIII:

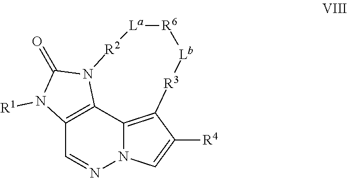

or a pharmaceutically acceptable salt thereof.

106. A compound selected from Table 1A, Table 1B, or Table 1C, or a pharmaceutically acceptable salt thereof.

107. A pharmaceutical composition comprising a compound of any one of embodiments 62-106, or a pharmaceutically acceptable salt thereof, and a pharmaceutically acceptable carrier.

108. A method of inhibiting JAK2 in a subject, comprising administering to the subject the compound of any one of embodiments 62-106 or the pharmaceutical composition of embodiment 107.

109. A method of treating a disease, disorder, or condition associated with JAK2, comprising administering to a subject in need thereof the compound of any one of embodiments 62-106 or the pharmaceutical composition of embodiment 107.

110. A method of treating cancer, comprising administering to a subject in need thereof the compound of any one of embodiments 62-106 or the pharmaceutical composition of embodiment 107.

111. A method of treating a hematological malignancy, comprising administering to a subject in need thereof the compound of any one of embodiments 62-106 or the pharmaceutical composition of embodiment 107.

112. The method of embodiment 111, wherein the hematological malignancy is leukemia or lymphoma.

113. A method of treating a myeloproliferative neoplasm, comprising administering to a subject in need thereof the compound of any one of embodiments 62-106 or the pharmaceutical composition of embodiment 107.

114. The method of embodiment 113, wherein the myeloproliferative neoplasm is polycythemia vera, essential thrombocytopenia, or myelofibrosis.

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein.

Certain abbreviations are utilized in the Examples below:

EA=ethyl acetate;
DMF=N,N-dimethylformamide;
DCM=Dichloromethane;
THF=Tetrahydrofuran;
LAH=Lithium aluminium hydride;
DME=1,2-Dimethoxyethane;
TEA=Triethylamine;
EIPEA=Ethyldiisopropylamine;
h=hour(s);
min=minute(s);
g=gram(s);
mg=milligram(s);
mol=mole(s);
mmol=millimole(s);
equiv=equivalents;
L=liters;
mL=milliliters;
M=molarity/molar;
N=normal;
μm=micrometer(s);
HATU=2-(7-Aza-1H-benzotriazole-1-yl)-1,1,3,3-tetramethyluronium hexafluorophosphate;
EDCI=N-(3-dimethylaminopropyl)-N′-ethylcarbodiimide hydrochloride;
HOBt=1-Hydroxybenzotriazole;
HPLC=high performance liquid chromatography;
SFC=supercritical fluid chromatography;
DIBAL-H=Diisobutylaluminium hydride;
Bn=Benzyl;
DMSO=Dimethyl sulfoxide;
rt=room temperature;
DBU=1,8-Diazabicyclo[5.4.0]undec-7-ene;
NCS═N-Chlorosuccinimide;
LiHMDS=Lithium bis(trimethylsilyl)amide;
DMAP=4-Dimethylaminopyridine;
n-BuLi=n-Butyllithium;
CAN or MeCN=acetonitrile;
TCFH=Chloro-N,N,N′,N′-tetramethylformamidinium Hexafluorophosphate;
NMI=1-Methylimidazole;
° C.=degrees Celsius;
bp=boiling point;
DEA=diethanolamine.

### Preparation of Provided Compounds

### Example I-1: methyl ((1R,3R)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

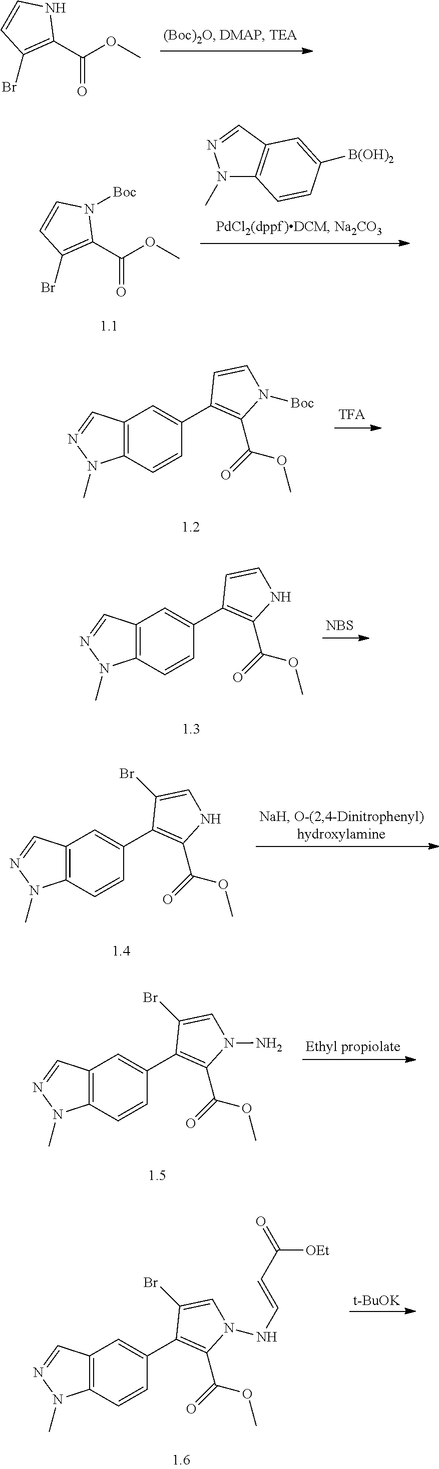

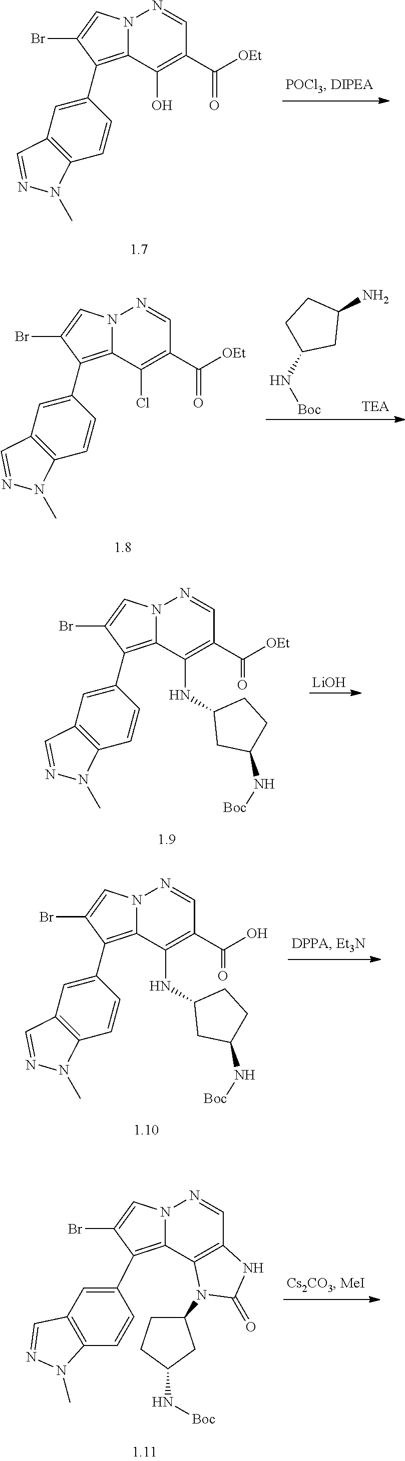

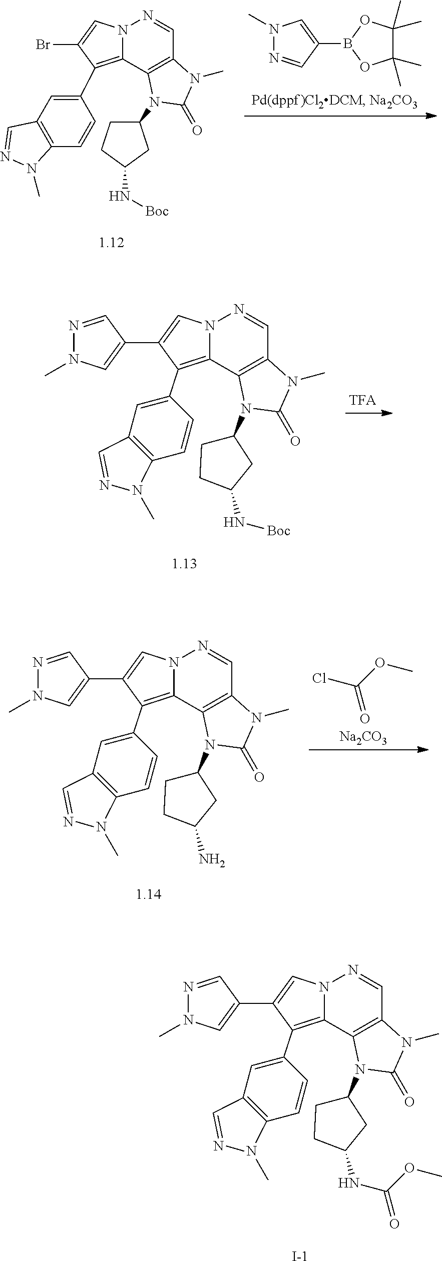

Synthesis of compound 1.1. To a solution of methyl 3-bromo-1H-pyrrole-2-carboxylate (20.0 g, 98.03 mmol, 1.0 equiv) in DCM (200 mL) was added 4-dimethylaminopyridine (1.19 g, 9.80 mmol, 0.1 equiv), triethylamine (29.70 g, 294 mmol, 3.0 equiv) and di-tert-butyldicarbonate (32.05 g, 110 mmol, 3.0 equiv) at 0° C. The reaction mixture was stirred at room temperature for 2 h. The reaction mixture was quenched with water and extracted with ethyl acetate. The combined organic layers were washed with brine, dried over anhydrous sodium sulfate, filtered, and concentrated under reduced pressure. The residue was purified by flash column chromatography on silica gel (10% ethyl acetate in hexane) to afford 1.1 (21.8 g, 73%). MS(ES): m/z 304 [M+H]⁺.

Synthesis of compound 1.2. A mixture of 1.1 (21.8 g, 71.7 mmol, 1.0 equiv), (1-methyl-1H-indazol-5-yl)boronic acid (25.3 g, 143.8 mmol, 2.0 equiv), sodium carbonate (22.8 g, 215.8 mmol, 3.0 equiv) in 1,4-dioxane (440 mL) and water (60 mL) was degassed by bubbling through a stream of argon for 10 min. [1,1′-Bis(diphenylphosphino)ferrocene]dichloropalladium(II), complex with dichloromethane (5.87 g, 7.17 mmol, 0.1 equiv) was added, and degassed for 5 min. The reaction mixture was stirred at 100° C. for 2 h. It was cooled to room temperature, filtered through a pad of Celite®, and rinsed with ethyl acetate. The filtrate was transferred into water and extracted with ethyl acetate. The combined organic layers were washed with brine, dried over anhydrous sodium sulfate, filtered and concentrated under reduced pressure. The residue was purified by flash column chromatography on silica gel (15% ethyl acetate in hexane) to afford 1.2 (14.8 g, 58%). MS(ES): m/z 356.1 [M+H]⁺.

Synthesis of compound 1.3. To a solution of 1.2 (14.8 g, 41.62 mmol, 1.0 equiv) in DCM (150 mL) was added trifluoroacetic acid (50 mL) at 0° C. for dropwise. The reaction mixture was stirred at room temperature for 1 h. The reaction mixture was transferred into a saturated sodium bicarbonate solution and extracted with dichloromethane. The combined organic layers were washed with brine, dried over anhydrous sodium sulfate, filtered and concentrated under reduced pressure to afford 1.3 (10.4 g, 98%). It was used in the next step without purification. MS(ES): m/z 256.10 [M+H]⁺.

Synthesis of compound 1.4. To a solution of 1.3 (10.4 g, 40.78 mmol, 1.0 equiv) in THF (166 mL) and water (41.6 mL) was added N-bromosuccinimide (7.25 g, 40.78 mmol, 1.0 equiv) at 0° C. in portions. The reaction mixture was stirred at 0° C. for 30 min. The reaction mixture was quenched with a saturated solution of sodium thiosulphate and extracted with DCM. The combined organic layers were washed with brine, dried over anhydrous sodium sulfate, filtered, and concentrated under reduced pressure. The residue was purified by flash column chromatography on silica gel (25% ethyl acetate in hexane) to afford 1.4 (10.8 g, 79%). MS(ES): m/z 335.1 [M+H]⁺.

Synthesis of compound 1.5. To a solution of 1.4 (10.8 g, 32.4 mmol, 1.0 equiv) in DMF was added sodium hydride (60% dispersion in mineral oil, 1.94 g, 48.6 mmol, 1.5 equiv) in portions at 0° C. It was stirred for 30 min and added O-(2,4-dinitrophenyl) hydroxylamine (9.68 g, 48.6 mmol, 1.5 equiv). The reaction mixture was stirred at room temperature for 1 h. The reaction mixture was transferred into ice-water and extracted with ethyl acetate. The combined organic layers were washed with brine, dried over anhydrous sodium sulfate, filtered and concentrated under reduced pressure. The residue was purified by flash column chromatography on silica gel (30% ethyl acetate in hexane) to afford 1.5 (10.5 g, 93%). MS(ES): m/z 350.3 [M+H]⁺.

Synthesis of compound 1.6. To a solution of 1.5 (10.5 g, 3.01 mmol, 1.0 equiv) in methanol (1000 mL) was added ethyl propiolate (36.9 g, 362.0 mmol, 12.0 equiv) at room temperature. The reaction mixture was stirred at 70° C. for 16 h. The reaction mixture was concentrated under reduced pressure. The residue was purified by flash column chromatography on silica gel (40% ethyl acetate in hexane) to afford 1.6 (8.9 g, 66%). MS(ES): m/z 448.2 [M+H]⁺.

Synthesis of compound 1.7. To a solution of 1.6 (8.9 g, 19.9 mmol, 1.0 equiv) in ethanol (150 mL) was added potassium tert-butoxide (4.47 g, 39.9 mmol, 2.0 equiv) at room temperature. The reaction mixture was stirred at 100° C. for 2 h. It was concentrated under reduced pressure. The residue was poured into ice-water. The solid precipitates were filtered, washed with water and dried under reduced pressure to afford 1.7 (5.2 g, 63%). MS(ES): m/z 416.5 [M+H]⁺.

Synthesis of compound 1.8. To a solution of 1.7 (5.2 g, 12.5 mmol, 1.0 equiv) in POCl₃ (104 mL, 20 v) at 0° C. was added DIPEA (49.55 g, 125 mmol, 10.0 equiv) dropwise and the mixture was allowed to stir at 100° C. for 16 h. The reaction mixture was poured into ice-water and quenched with a saturated solution of sodium bicarbonate. It was extracted with ethyl acetate. The combined organic layers were washed with brine, dried over anhydrous sodium sulfate, filtered and concentrated under reduced pressure. The residue was purified by flash column chromatography on silica gel (30% ethyl acetate in hexane) to afford 1.8 (2.8 g, 64%). MS(ES) m/z 434.1 [M+H]⁺.

Synthesis of compound 1.9. A solution of 1.8 (2.8 g, 6.48 mmol, 1.0 equiv), tert-butyl ((1R,3R)-3-aminocyclopentyl)carbamate (1.5 g, 7.7 mmol, 1.2 equiv) and triethylamine (2.7 mL, 19.4 mmol, 3.0 equiv) in THF (74 mL) was stirred at 70° C. for 1 h. The reaction mixture was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 50% ethyl acetate in hexane) to afford 1.9 (2.6 g, 68%). MS(ES): m/z 598.1 [M+H]⁺.

Synthesis of compound 1.10. To a solution of 1.9 (2.6 g, 4.6 mmol, 1.0 equiv) in 33 mL mixture of THF:MeOH:H₂O (2:2:1) was added lithium hydroxide hydrate (3.66 g, 92 mmol, 20.0 equiv) at room temperature and stirred for 3 h. It was transferred into ice-water and adjusted pH ˜5 to 6 with 1 N HCl and extracted with 10% methanol in DCM. The combined organic layers were washed with brine, dried over anhydrous sodium sulfate, filtered and concentrated under reduced pressure. The residue was triturated with pentane to afford 1.10 (1.9 g, 77%). MS(ES): m/z 570.4 [M+H]⁺.

Synthesis of compound 1.11. To a solution of 1.10 (1.9 g, 3.3 mmol, 1.0 equiv) in toluene (30 mL) was added diphenyl phosphoryl azide (1.1 g, 4.0 mmol, 1.2 equiv) and triethylamine (7.4 mL, 53.5 mmol, 1.6 equiv) at room temperature. The reaction mixture was stirred at 120° C. for 3 h. The reaction mixture was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 1.8% methanol in DCM) to afford 1.11 (0.850 g, 45%). MS(ES): m/z 567.5 [M+H]⁺.

Synthesis of compound 1.12. To a solution of 1.11 (0.850 g, 1.50 mmol, 1.0 equiv) in DMF (10 mL) was added cesium carbonate (0.730 g, 2.0 mmol, 1.5 equiv) and methyl iodide (0.256 g, 1.8 mmol, 1.2 equiv) at 0° C. The reaction mixture was stirred at room temperature for 15 min. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (1.4% methanol in DCM) to afford 1.12 (0.690 g, 79%). MS(ES): m/z 581.2 [M+H]⁺.

Synthesis of compound 1.13. A mixture of 1.12 (0.250 g, 0.424 mmol, 1.0 equiv), 1-methyl-4-(4,4,5,5-tetramethyl-1,3,2-dioxaborolan-2-yl)-1H-pyrazole (0.176 g, 0.848 mmol, 2.0 equiv) and sodium carbonate (0.135 g, 1.272 mmol, 3.0 equiv) in 1,4-dioxane (8 mL) and water (2 mL) was degassed by bubbling through a stream of argon for 10 min. [1,1′-bis(diphenylphosphino)ferrocene]dichloropalladium(II) dichloromethane (0.035 g, 0.042 mmol, 0.1 equiv) was added and degassed for 5 min. The mixture was stirred at 100° C. for 2 h. It was cooled to room temperature, poured into water and extracted with ethyl acetate. The combined organic layers were washed with brine, dried over anhydrous sodium sulfate, filtered and concentrated under reduced pressure. The residue was purified by flash column chromatography on silica gel (0-2.3% methanol in DCM) to afford 1.13 (0.083 g, 33%). MS(ES): m/z 582.3

Synthesis of compound 1.14. To a solution of 1.13 (0.083 g, 0.14 mmol, 1.0 equiv) in DCM (4.0 mL) was added trifluoroacetic acid (1.5 mL) at 0° C. dropwise. The reaction mixture was stirred for 15 minutes at room temperature. Most solvent was removed under reduced pressure. The residue was triturated with n-pentane and the solids were dried under reduced pressure to afford 1.14 (0.061 g). It was used in next step without purification. MS(ES): 482.4 m/z [M+H]⁺.

Synthesis of I-1. To a solution of 1.14 (0.061 g, 0.126 mmol, 1.0 equiv) in DCM (4.0 mL) was added aqueous solution (2 mL) of sodium carbonate (0.040 g, 0.38 mmol, 3.0 equiv) at 0° C. and stirred reaction mixture for 10 min. Methyl chloroformate (0.024 g, 0.25 mmol, 2.0 equiv) was added dropwise and the reaction mixture was stirred at room temperature for 10 min. The reaction mixture was quenched in water and product extracted with ethyl acetate. The combined organic layers were washed with brine, dried over anhydrous sodium sulfate, filtered and concentrated under reduced pressure. The residue was purified by flash column chromatography on silica gel (Combiflash®, 0-2.6% methanol in DCM) to afford I-1 (0.040 g, 59%). MS(ES): 540.3 m/z [M+H]⁺; ¹H NMR (DMSO-d₆, 400 MHz): δ 8.39 (s, 1H), δ 8.39 (s, 1H), 8.15 (d, 1H), 8.08 (d, 1H), 7.78 (d, 2H), 7.42 (dd, 1H), 7.27 (d, 1H), 7.04 (s, 0.6H), 6.93 (s, 0.4H), 6.68 (m, 1H), 4.15 (s, 3H), 3.89-3.88 (m, 1H), 3.86-3.84 (m, 1H), 3.75 (s, 3H), 3.54 (s, 3H), 3.45 (s, 3H), 2.03 (m, 1H), 1.81 (m, 1H), 1.52 (m, 1H), 1.16-1.11 (m, 1H), 0.90-0.75 (m, 1H), 0.48-0.41 (m, 0.4H), −0.13-0.17 (m, 0.6H).

### Example I-2: methyl ((1R,3R)-3-(8-(3-fluoro-1-methyl-1H-pyrazol-4-yl)-3-methyl-9-(1-methyl-1H-indazol-5-yl)-2-oxo-2,3-dihydro-1H-imidazo[4,5-d]pyrrolo[1,2-b]pyridazin-1-yl)cyclopentyl)carbamate

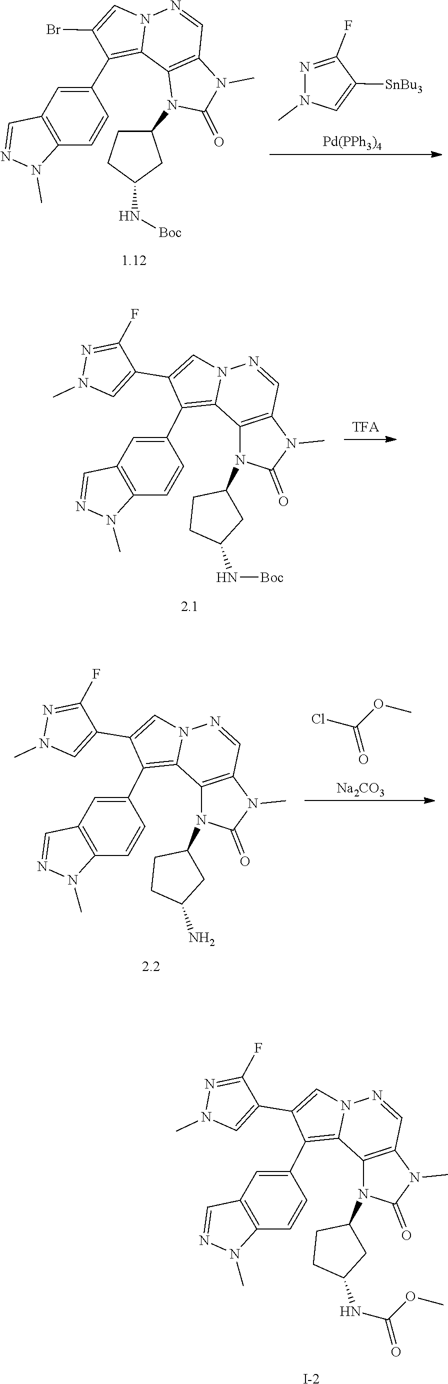

Synthesis of compound 2.1. A mixture of 1.12 (0.225 g, 0.38 mmol, 1.0 equiv) and 3-fluoro-1-methyl-4-(tributylstannyl)-1H-pyrazole (0.606 g, 1.55 mmol, 4.0 equiv) in 1,4-dioxane (10 mL) was degassed under argon atmosphere for 10 min. Tetrakis (Pd(PPh₃)₄) (0.045 g, 0.038 mmol, 0.1 equiv) was added to the reaction mixture and purged for 10 min. The mixture was stirred at 140° C. for 3 h. The reaction mixture was poured into water and extracted with ethyl acetate. The combined organic layers were washed with brine, dried over anhydrous sodium sulfate, filtered and concentrated under reduced pressure. The residue was purified by flash column chromatography on silica gel (Combiflash®, 0-3.2% methanol in DCM) to afford 2.1 (0.082 g, 35%). MS(ES): m/z 600.3 [M+H]⁺.

Synthesis of compound 2.2. To a solution of 2.1 (0.082 g, 0.136 mmol, 1.0 equiv) in DCM (5.0 mL) at 0° C. was dropwise added trifluoroacetic acid (1.5 mL) and stirred for 15 minutes at room temperature. Most solvent was removed under reduced pressure. The residue was triturated with pentane and the solids were dried under reduced pressure to afford 2.2 (0.059 g). It was used in next step without purification. MS(ES): 500.2 m/z [M+H]⁺.

Synthesis of I-2. Compound I-2 was prepared from 2.2 following the procedure described in the synthesis of compound I-1. The product was purified by flash column chromatography on silica gel (Combiflash®, 0-2.6% methanol in DCM). MS(ES): 558.3 m/z [M+H]⁺, ¹H NMR (DMSO-d₆, 400 MHz): δ 8.43 (s, 1H), 8.06 (d, 1H), 7.97 (m, 1H), 7.74 (m, 2H), 7.39-7.35 (m, 1H), 6.71 (s, 1H), 6.66 (s, 0.5H), 6.34 (s, 0.5H), 4.13 (s, 3H), 3.89-3.88 (m, 1H), 3.86-3.84 (m, 1H), 3.74 (s, 3H), 3.52 (s, 3H), 3.44 (s, 3H), 2.03-1.97 (m, 1H), 1.81-1.79 (m, 1H), 1.53 (m, 1H), 1.24-(m, 1H), 0.85-0.73 (m, 1H), 0.32 (s, 0.5H), −0.19 (s, 0.5H).

### Example I-3: methyl ((1s,3s)-3-(8-(3-fluoro-1-(methyl-d₃)-1H-pyrazol-4-yl)-3-methyl-9-(1-(methyl-d₃)-1H-indazol-5-yl)-2-oxo-2,3-dihydro-1H-imidazo[4,5-d]pyrrolo[1,2-b]pyridazin-1-yl)cyclobutyl)carbamate

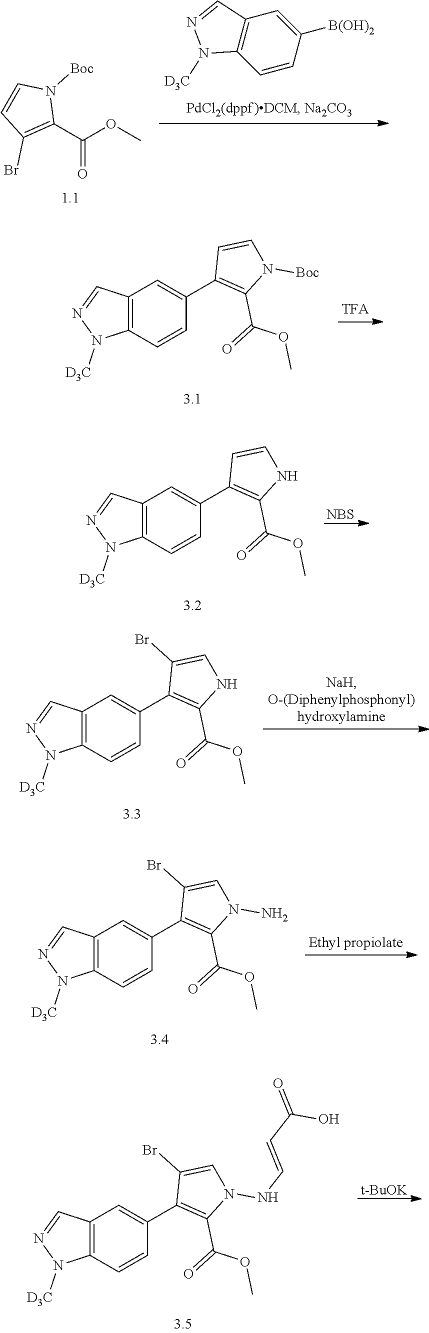

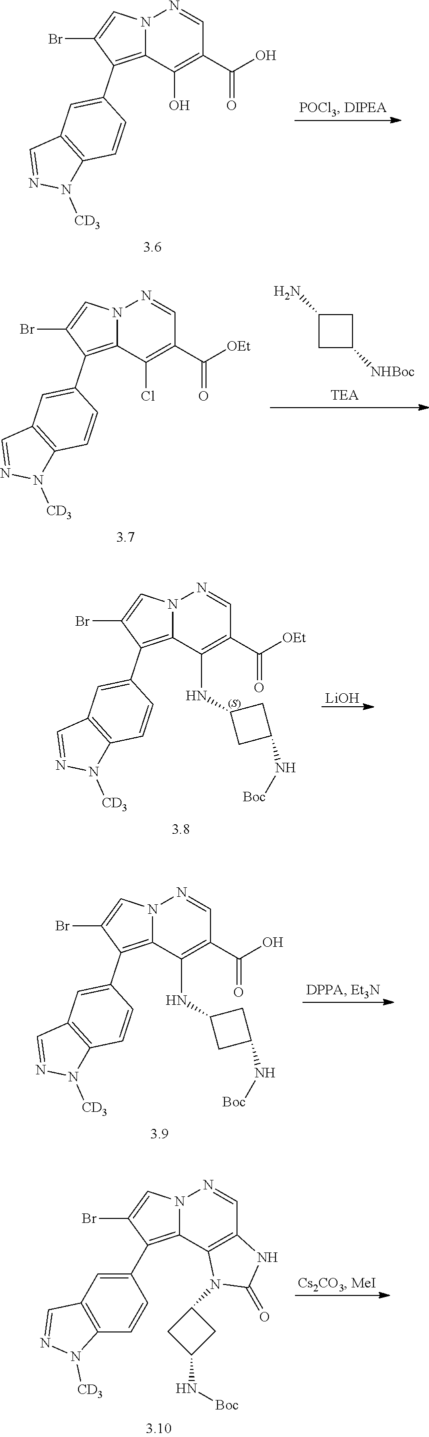

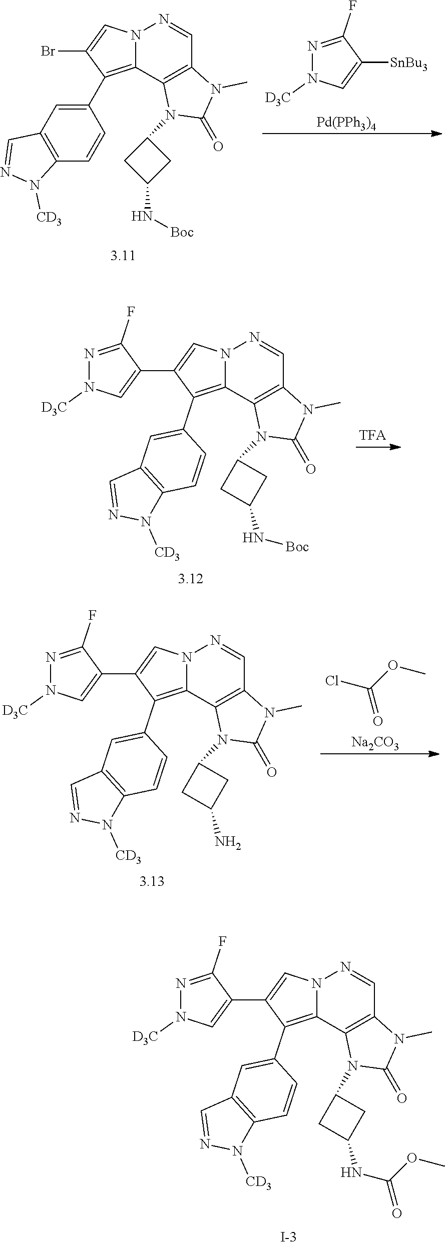

Synthesis of compound 3.1. Compound 3.1 was prepared from 1.1 and (1-(methyl-d₃)-1H-indazol-5-yl)boronic acid following the procedure described in the synthesis of compound 1.2. The product was purified by flash column chromatography on silica gel (15% ethyl acetate in hexane). MS(ES): m/z 359.2 [M+H]⁺.

Synthesis of compound 3.2. Compound 3.2 was prepared from 3.1 following the procedure described in the synthesis of compound 1.3. The product was used in the next step without purification. MS(ES): m/z 259.1 [M+H]⁺.

Synthesis of compound 3.3. Compound 3.3 was prepared from 3.2 following the procedure described in the synthesis of compound 1.4. The product was purified by flash column chromatography on silica gel (10-30% ethyl acetate in hexane). MS(ES): m/z 338.2 [M+H]⁺.

Synthesis of compound 3.4. Compound 3.4 was prepared from 3.3 following the procedure described in the synthesis of compound 1.5. The product was purified by flash column chromatography on silica gel (30% ethyl acetate in hexane). MS(ES): m/z 353.1 [M+H]⁺.

Synthesis of compound 3.5. Compound 3.5 was prepared from 3.4 following the procedure described in the synthesis of compound 1.6. The product was purified by flash column chromatography on silica gel (40% ethyl acetate in hexane). MS(ES): m/z 451.4 [M+H]⁺.

Synthesis of compound 3.6. Compound 3.6 was prepared from 3.5 following the procedure described in the synthesis of compound 1.7. The product was used in next step without further purification. MS(ES): m/z 419.4 [M+H]⁺.

Synthesis of compound 3.7. Compound 3.7 was prepared from 3.6 following the procedure described in the synthesis of compound 1.8. The product was purified by flash column chromatography on silica gel (30% ethyl acetate in hexane). MS(ES): m/z 437.5 [M+H]⁺.

Synthesis of compound 3.8. Compound 3.8 was prepared from 3.7 and tert-butyl ((1s,3s)-3-aminocyclobutyl)carbamate following the procedure described in the synthesis of compound 1.9. The product was purified by flash column chromatography on silica gel (Combiflash®, 0-40% ethyl acetate in hexane). MS(ES): m/z 587.3 [M+H]⁺.

Synthesis of compound 3.9. Compound 3.9 was prepared from 3.8 following the procedure described in the synthesis of compound 1.10. The product was purified by trituration with pentane. MS(ES): m/z 559.1 [M+H]⁺.

Synthesis of compound 3.10. Compound 3.10 was prepared from 3.9 following the procedure described in the synthesis of compound 1.11. The product was purified by flash column chromatography on silica gel (Combiflash®, 0-3% methanol in DCM). MS(ES): m/z 556.2 [M+H]⁺.

Synthesis of compound 3.11. Compound 3.11 was prepared from 3.10 following the procedure described in the synthesis of compound 1.12. The product was purified by flash column chromatography on silica gel (0-3% methanol in DCM). MS(ES): m/z 570.2 [M+H]⁺.

Synthesis of compound 3.12. A mixture of 3.11 (0.250 g, 0.43 mmol, 1.0 equiv) and 3-fluoro-1-(methyl-d₃)-4-(tributylstannyl)-1H-pyrazole (0.860 g, 21.94 mmol, 5.0 equiv) in 1,4-dioxane (10 mL) was degassed by bubbling through a stream of argon for 10 min. Tetrakis(Pd(PPh₃)₄) (0.051 g, 0.043 mmol, 0.1 equiv) was added and purged for 10 min. The reaction mixture was stirred at 140° C. for 3 h. It was poured into water and extracted with ethyl acetate. The combined organic layers were washed with brine, dried over anhydrous sodium sulfate, filtered and concentrated under reduced pressure. The residue was purified by flash column chromatography on silica gel (Combiflash®, 2.2% methanol in DCM). MS(ES): m/z 592.1 [M+H]⁺.

Synthesis of compound 3.13. Compound 3.13 was prepared from 3.12 following the procedure described in the synthesis of compound 1.14. The product was triturated with n-pentane and dried under reduced pressure. It was used in next step without further purification. MS(ES): 492.2 m/z [M+H]⁺.

Synthesis of I-3. Compound I-3 was prepared from 3.13 following the procedure described in the synthesis of compound I-1. The product was purified by flash column chromatography on silica gel (Combiflash®, 3.2% methanol in DCM). MS(ES): 550.4 m/z [M+H]⁺; ¹H NMR (DMSO-d₆, 400 MHz): δ 8.44 (s, 1H), 8.09 (s, 1H), 8.19 (s, 1H), 7.97 (s, 1H), 7.83 (d, J=8.8 Hz, 1H), 7.75 (s, 1H), 7.28-7.26 (m, 1H), 6.80 (s, 1H), 3.56 (s, 3H), 3.43 (s, 3H), 3.05-3.01 (m, 1H), 2.66-2.58 (m, 1H), 2.16-2.14 (m, 1H), 1.76 (s, 1H), 1.35-1.33 (m, 1H), 1.28-1.14 (m, 1H).

### Example I-4-a and I-4-b: methyl ((1S,2S)-2-(8-(3-fluoro-1-(methyl-d₃)-1H-pyrazol-4-yl)-3-methyl-9-(1-(methyl-d₃)-1H-indazol-5-yl)-2-oxo-2,3-dihydro-1H-imidazo[4,5-d]pyrrolo[1,2-b]pyridazin-1-yl)cyclopropyl)carbamate and methyl ((1R,2R)-2-(8-(3-fluoro-1-(methyl-d₃)-1H-pyrazol-4-yl)-3-methyl-9-(1-(methyl-d₃)-1H-indazol-5-yl)-2-oxo-2,3-dihydro-1H-imidazo[4,5-d]pyrrolo[1,2-b]pyridazin-1-yl)cyclopropyl)carbamate

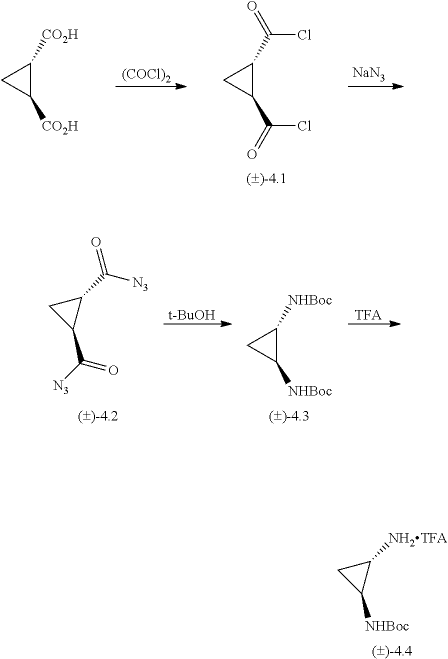

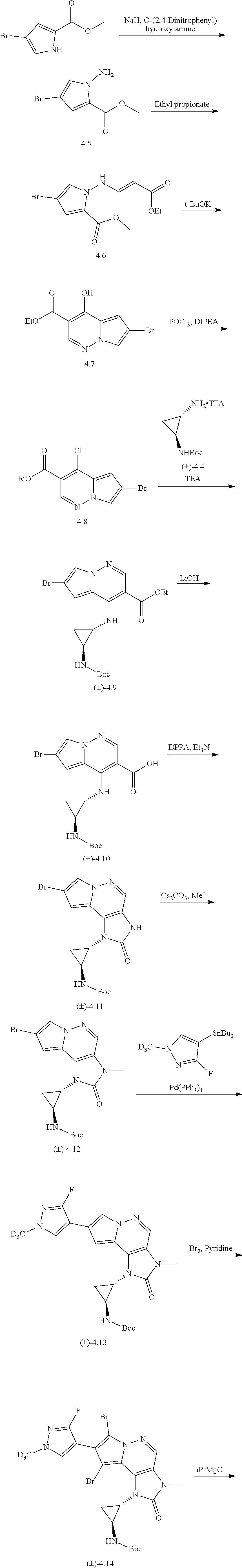

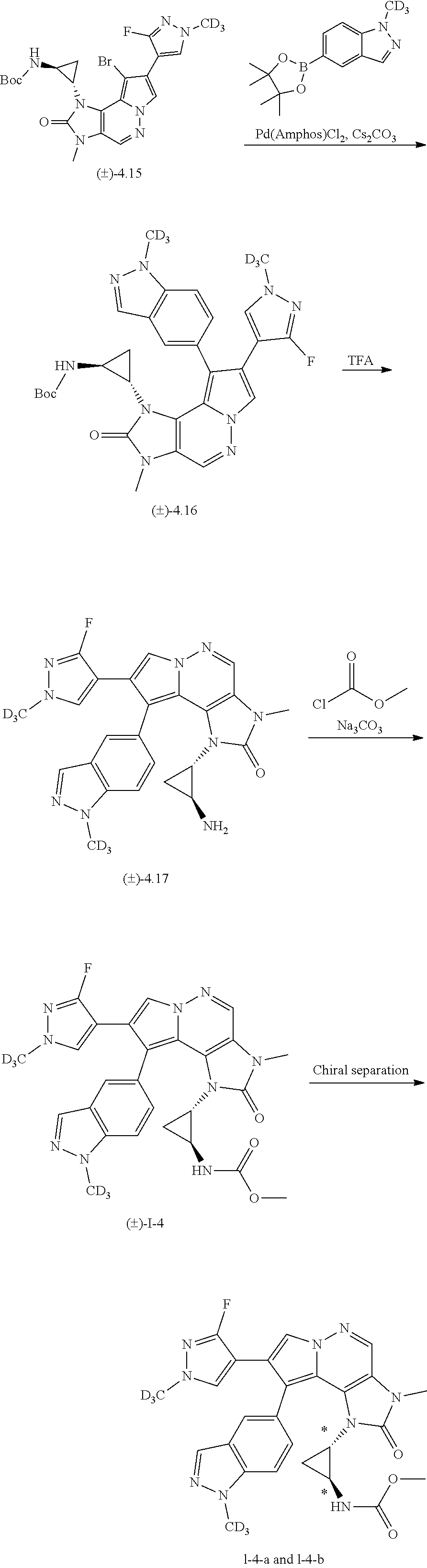

Synthesis of compound (±)-4.1. To a solution of trans-cyclopropane-1,2-dicarboxylic acid (20 g, 153.73 mmol, 1.0 equiv) in DCM (300 mL) at 0° C. was added oxalyl chloride (48.75 g, 384.61 mmol, 2.5 equiv) dropwise followed by catalytic amount of DMF. The reaction mixture was stirred at room temperature for 1 h. It was concentrated under reduced pressure to afford (±)-4.1 (20 g). The residue was used in the next step without purification.

Synthesis of compound (±)-4.2. To a solution of (±)-4.1 (20 g, 119.77 mmol, 1.0 equiv) in acetone (100 mL) at −40° C. was added dropwise aqueous solution (200 mL) of sodium azide (27.24 g, 419.16 mmol, 3.5 equiv). The reaction mixture was stirred at room temperature for 45 min. It was transferred into water and extracted with diethyl ether. The organic layer was washed with water, brine, dried over anhydrous sodium sulfate and filtered. The filtrate was concentrated to half of the volume under reduced pressure. The residue was diluted with toluene (100 mL), and again partially concentrated under reduced pressure to remove traces of diethyl ether. The product was used in the next step without purification.

Synthesis of compound (±)-4.3. The solution of (±)-4.2 in toluene (100 mL) was stirred at 80° C. for 2 h until the evolution of nitrogen gas ceased completely. (Caution: the reaction was highly exothermic.) Tert-butanol (80.0 mL) was added dropwise to the reaction mixture at 80° C. and stirred for 2 h. It was cooled to ambient temperature and concentrated under reduced pressure. The residue was purified by flash column chromatography on silica gel (35% ethyl acetate in hexane) to afford (±)-4.3 (15.00 g, 51%). MS(ES): m/z 273.3 [M+H]⁺.

Synthesis of compound (±)-4.4. To a solution of (±)-4.3 (15.0 g, 55.14 mmol, 1.0 equiv) in diethyl ether (200 mL) at 0° C. was added trifluoroacetic acid (84.38 mL, 1102.94 mmol, 20.0 equiv). The reaction mixture was stirred at room temperature for 16 h. Most solvent was removed under reduced pressure to afford (±)-4.4 (4.60 g). It was used in the next step without purification.

Synthesis of compound 4.5. To a solution of methyl 4-bromo-1H-pyrrole-2-carboxylate (25 g, 122.54 mmol, 1.0 equiv) in DMF (1.25 L) was added sodium hydride (60% dispersion in oil, 3.52 g, 147.05 mmol, 1.2 equiv) at 0° C. and allowed to stir for 30 min. To the mixture was added O-(2,4-dinitrophenyl) hydroxylamine (29.26 g, 147.05 mmol, 1.2 equiv) and stirred at room temperature for 3 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (23% ethyl acetate in hexane) to afford 4.5 (20 g, 74%). MS(ES): m/z 220.6 [M+H]⁺.

Synthesis of compound 4.6. To a solution of 4.5 (20.0 g, 91.32 mmol, 1.0 equiv) in methanol (1 L) was added ethyl propionate (107.39 g, 1095.89 mmol, 12.0 equiv) at room temperature. The mixture was heated to reflux for 16 h. Most solvent was removed under reduced pressure. The residue was purified by flash column chromatography on silica gel (25% ethyl acetate in hexane) to afford 4.6 (13 g, 45%). MS(ES): m/z 318.3 [M+H]⁺.

Synthesis of compound 4.7. To a solution of 4.6 (13.0 g, 41.00 mmol, 1.0 equiv) in ethanol (160 mL) was added potassium tert-butoxide (9.18 g, 82.01 mmol, 2.0 equiv) at room temperature. The reaction mixture was stirred at 90° C. for 3 h. It was poured on to ice-water and stirred for 15 min. The precipitated solids were collected by filtration and dried under vacuum to afford 4.7 (9.2 g, 79%). MS(ES): m/z 286.3 [M+H]⁺.

Synthesis of compound 4.8. To a solution of 4.7 (9.2 g, 32.28 mmol, 1.0 equiv) in phosphorus oxychloride (78.5 mL) at 0° C. was added N,N-diisopropylethylamine (7.1 mL). The mixture was stirred at 90° C. for 3 h. It was transferred into ice-water, basified with saturated sodium bicarbonate solution and extracted with dichloromethane. The combined organic layers were washed with brine, dried over anhydrous sodium sulfate, filtered and concentrated under reduced pressure. The residue was purified by flash column chromatography on silica gel (10% ethyl acetate in hexane) to afford 4.8 (6.0 g, 61%). MS(ES): m/z 303.5 [M+H]⁺, 305.2 [M+H]⁺.

Synthesis of compound (±)-4.9. To a solution of 4.8 (5.2 g, 17.16 mmol, 1.0 equiv) in acetonitrile (60 mL) was added (±)-4.4 (4.4 g, 25.70 mmol, 1.5 equiv) followed by triethylamine (5.19 g, 51.48 mmol, 3.0 equiv). The reaction mixture was stirred at 70° C. for 2 h. It was transferred into water and extracted with ethyl acetate. The combined organic layers were washed with brine, dried over anhydrous sodium sulfate, filtered and concentrated under reduced pressure. The residue was purified by flash column chromatography on silica gel (7% ethyl acetate in hexane) to afford (±)-4.9 (4.30 g, 57%). MS(ES): m/z 439.1 [M+H]⁺, 441.1 [M+H]⁺.

Synthesis of compound (±)-4.10. To a solution of (±)-4.9 (4.30 g, 9.79 mmol, 1.0 equiv) in ethanol (45 mL) and THF (22 mL) was added lithium hydroxide monohydrate (8.2 g, 195.8 mmol, 20 equiv) in H₂O (20 mL). The reaction mixture was stirred at 60° C. for 2 h. It was concentrated under reduced pressure. The residue was acidified with 1N HCl and solids were precipitated. The solids were collected by filtration, rinsed with water and dried under reduce pressure to afford (±)-4.10 (3.7 g, 92%) MS(ES): m/z 411.1 [M+H]⁺, 413.1 [M+H]⁺.

Synthesis of compound (±)-4.11. To a solution of (±)-4.10 (3.70 g, 9.00 mmol, 1.0 equiv) in toluene was added triethylamine (1.45 g, 14.40 mmol, 1.6 equiv) followed by diphenylphosphoryl azide (2.97 g, 10.80 mmol, 1.2 equiv) at 0° C. The reaction mixture was stirred at 120° C. for 2 h. The reaction mixture was transferred into water and extracted with ethyl acetate. The combined organic layers were washed with brine, dried over anhydrous sodium sulfate, filtered and concentrated under reduced pressure. The residue was purified by flash column chromatography on silica gel (2.5% methanol in DCM) to afford (±)-4.11 (2.8 g, 76%) MS(ES): m/z 408.2 [M+H]⁺, 410.2 [M+H]⁺.

Synthesis of compound (±)-4.12. To a solution of (±)-4.11 (2.80 g, 6.86 mmol, 1.0 equiv) in DMF (40 mL) was slowly added cesium carbonate (3.3 g, 1.02 mmol, 1.5 equiv) at 0° C. followed by methyl iodide (1.16 g, 8.02 mmol, 1.2 equiv). The reaction mixture was stirred at room temperature for 1 h. It was poured into ice-water and solids were precipitated. The solids were collected by filtration, rinsed with water and dried under vacuum to afford (±)-4.12 (2.10 g, 73%). MS(ES): m/z 422.1 [M+H]⁺, 424.1 [M+H]⁺.

Synthesis of compound (±)-4.13. A mixture of (±)-4.12 (2.1 g, 4.97 mmol, 1.0 equiv) and 3-fluoro-1-(methyl-d₃)-4-(tributylstannyl)-1H-pyrazole (9.75 g, 24.86 mmol, 5.0 equiv) in 1,4-dioxane (35 mL) was degassed by bubbling through a stream of argon for 10 min. Tetrakis(triphenylphosphine) palladium (0) (0.574 g, 0.497 mmol, 0.1 equiv) was added to the reaction mixture and again purged for 10 min. The mixture was stirred at 140° C. for 1 h. It was poured into water and extracted with ethyl acetate. The combined organic layers were washed with brine, dried over anhydrous sodium sulfate, filtered and concentrated under reduced pressure. The residue was purified by flash column chromatography on silica gel (1.8% methanol in DCM) to afford (±)-4.13 (0.48 g, 22%). MS(ES): m/z 445.2 [M+H]⁺.

Synthesis of compound (±)-4.14. To a solution of (±)-4.13 (0.48 g, 1.08 mmol, 1.0 equiv) in chloroform (20 mL) was added pyridine (0.512 g, 6.486 mmol, 6.0 equiv) at −10° C. A solution of bromine (0.171 g, 2.162 mmol, 2.0 equiv) in chloroform was added dropwise. The reaction mixture was stirred at −10° C. for 5 min. It was transferred into a saturated sodium bicarbonate solution and extracted with dichloromethane. The combined organic layers were washed with brine, dried over anhydrous sodium sulfate, filtered and concentrated under reduced pressure. The residue was purified by flash column chromatography on silica gel (Combiflash®, 2.0% methanol in DCM) to afford (±)-4.14 (0.370 g, 60%). MS(ES): m/z 601.3 [M+H]⁺, m/z 603.3 [M+H]⁺.

Synthesis of compound (±)-4.15. To a solution of (±)-4.14 (0.370 g, 0.614 mmol, 1.0 equiv) in THF (8 mL) at −10° C. was added a solution of isopropylmagnesium chloride (2 M in THF, 1.84 g, 3.68 mmol, 6.0 equiv). The mixture was stirred at −10° C. for 15 min. It was transferred into saturated ammonium chloride solution and product extracted with ethyl acetate. The combined organic layers were washed with brine, dried over anhydrous sodium sulfate, filtered and concentrated under reduced pressure. The residue was purified by flash column chromatography on silica gel (Combiflash®, 2.2% methanol in DCM) to afford (±)-4.15 (0.310 g, 96%). MS(ES): m/z 523.2 [M+H]⁺, 525.2 [M+H]⁺.

Synthesis of compound (±)-4.16. A mixture of (±)-4.15 (0.310 g, 0.592 mmol, 1.0 equiv), 1-(methyl-d₃)-5-(4,4,5,5-tetramethyl-1,3,2-dioxaborolan-2-yl)-1H-indazole (0.159 g, 0.888 mmol, 1.5 equiv) and cesium carbonate (0.578 g, 1.778 mmol, 3.0 equiv) in 1,2-dimethoxyethane (6 mL) and water (1.5 mL) was degassed by bubbling through a stream of argon for 10 min. Bis[di-tert-butyl (4-dimethylaminophenyl) phosphine]dichloropalladium(II) (0.042 g, 0.059 mmol, 0.1 equiv) was added and degassed for 5 min. The reaction mixture was stirred at 110° C. for 1 h. It was poured into water and extracted with ethyl acetate. The combined organic layers were washed with brine, dried over anhydrous sodium sulfate, filtered and concentrated under reduced pressure. The residue was purified by flash column chromatography on silica gel (Combiflash®, 2.8% methanol in DCM) to afford (±)-4.16 (0.115 g, 34%). MS(ES): m/z 578.4 [M+H]⁺.

Synthesis of compound (±)-4.17. To a solution of (±)-4.16 (0.115 g, 0.199 mmol, 1.0 equiv) in DCM (5.0 mL) at 0° C. was added trifluoroacetic acid (1 mL) and stirred for 15 min at room temperature. Most solvent was removed under reduced pressure to afford (±)-4.17 (0.110 g). MS(ES): 478.3 m/z [M+H]⁺. The product was used in the next step without purification.

Synthesis of compound (±)—I-4. To a solution of (±)-4.17 (0.110 g, 0.175 mmol, 1.0 equiv) in DCM (3.0 mL) was added aqueous solution of sodium carbonate (2 mL) and stirred reaction mixture at 0° C. for 10 min. Methyl chloroformate (0.054 g, 0.575 mmol, 2.5 equiv) was added dropwise and the reaction mixture stirred at same temperature for 10 min. It was poured into water and extracted with ethyl acetate. The combined organic layers were washed with brine, dried over anhydrous sodium sulfate, filtered and concentrated under reduced pressure. The residue was purified by flash column chromatography on silica gel (Combiflash®, 3.2% methanol in DCM) to afford (±)—I-4 (0.062 g, 50%). MS(ES): 536.4 m/z [M+H]⁺.

Synthesis of I-4-a and I-4-b. The racemate was separated by SFC (column: CHIRALPAK AD-H (250*30 mm), 5 μm; mobile phase: (A) Liquid CO₂. (B) 0.1% NH₃ in propane-2-ol: ACN (50:50); flow rate: 80 mL/min) to afford first eluting fraction (I-4-a) and second eluting fraction (I-4-b). *The absolute configuration of the chiral center is not determined.

I-4-a: MS(ES): m/z: 536.35 [M+H]⁺, ¹H NMR (DMSO-d₆, 400 MHz): δ 8.43 (s, 1H), 8.03-8.01 (d, J=9.6 Hz 1H), 7.94 (s, 1H), 7.71-7.69 (d, J=6.8 Hz, 1H), 7.62-7.57 (d, J=8 Hz, 1H), 7.37 (s, 1H), 7.16 (bs, 1H), 6.98 (s, 1H), 3.37 (s, 3H), 3.34 (s, 3H), 2.83 (bs, 0.5H), 2.67 (bs, 0.5H), 2.23 (bs, 0.5H), 2.14 (bs, 0.5H), 0.79 (bs, 0.5H), 0.71 (bs, 0.5H), −0.48 (bs, 0.5H), −0.58 (bs, 0.5H).

I-4-b: MS(ES): m/z: 536.32 [M+H]⁺, ¹H NMR (DMSO-d₆, 400 MHz): δ 8.43 (s, 1H), 8.01 (d, J=9.6 Hz 1H), 7.94 (s, 1H), 7.71-7.69 (d, J=6.8 Hz, 1H), 7.62-7.57 (d, J=8 Hz, 1H), 7.37 (s, 1H), 7.16 (bs, 1H), 6.98 (s, 1H), 3.39 (s, 3H), 3.36 (s, 3H), 2.84 (bs, 0.5H), 2.66 (bs, 0.5H), 2.22 (bs, 0.5H), 2.14 (bs, 0.5H), 0.79 (bs, 0.5H), 0.70 (bs, 0.5H), −0.48 (bs, 0.5H), −0.59 (bs, 0.5H).

### Example I-5: (1R,3S)—N,N-dimethyl-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentane-1-carboxamide

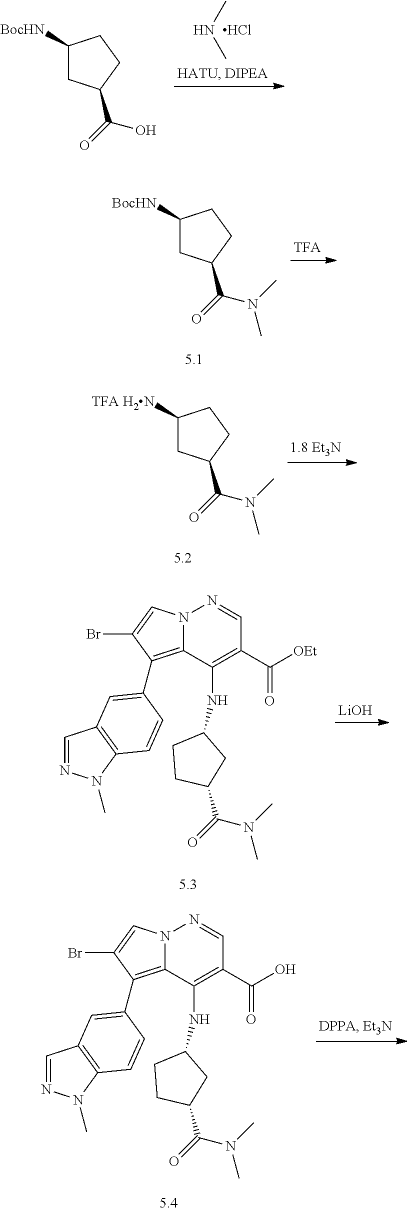

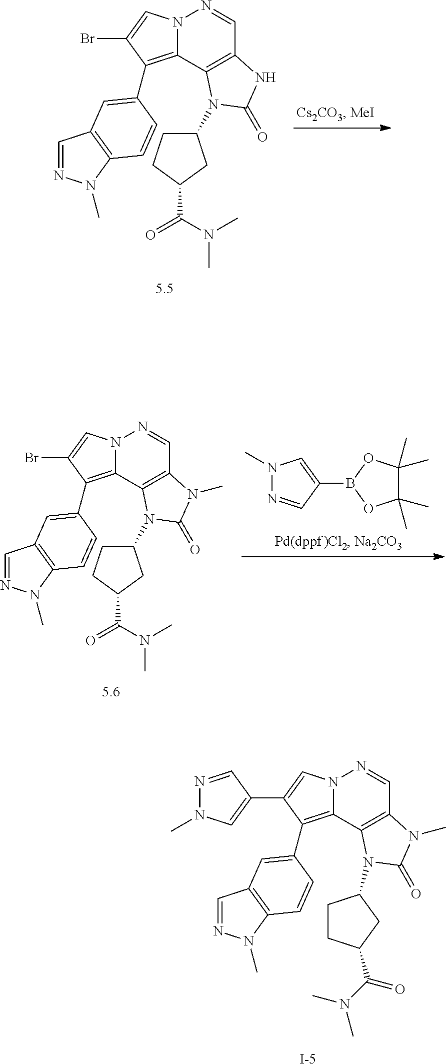

Synthesis of compound 5.1. To a solution of (1R,3S)-3-((tert butoxycarbonyl)amino)cyclopentane-1-carboxylic acid (2.00 g, 8.73 mmol, 1.0 equiv) in DMF (20 mL) was added HATU (4.97 g, 13.1 mmol, 1.5 equiv) at 0° C. and stirred for 30 min. Diisopropylethylamine (3.38 g, 26.2 mmol, 3.0 equiv) and dimethylamine hydrochloride (1.06 g, 13.1 mmol, 1.5 equiv) were added. The reaction mixture was stirred at room temperature for 2 h and was quenched with water and extracted with ethyl acetate. The combined organic layers were washed with brine, dried over anhydrous sodium sulfate, filtered and concentrated under reduced pressure. The residue was purified by flash column chromatography on silica gel (50% ethyl acetate in hexane) to afford 5.1 (1.60 g, 72%). MS(ES): m/z 257.35 [M+H]⁺.

Synthesis of compound 5.2. To a solution of 5.1 (1.6 g, 6.250 mmol, 1.0 equiv) in DCM (18 mL) was added trifluoroacetic acid (3.4 mL) at 0° C. dropwise. The reaction mixture was stirred at room temperature for 1 h. It was concentrated under reduced pressure. The residue was triturated with n-pentane and dried under reduced pressure to afford 5.2 (0.98 g, 62%). It was used in the next step without purification. MS(ES): m/z 157.25 [M+H]⁺.

Synthesis of compound 5.3. Compound 5.3 was prepared from compound 5.2 and 1.8 following the procedure described in the synthesis of compound 1.9. The product was purified by flash column chromatography on silica gel (Combiflash®, 50% ethyl acetate in hexane). MS(ES): m/z 553.5 and 555.4 [M+H]⁺.

Synthesis of compound 5.4. Compound 5.4 was prepared from compound 5.3 following the procedure described in the synthesis of compound 1.10. The product was triturated with pentane, dried under reduced pressure and used in the next step without purification. MS(ES): m/z 525.3/527.41 [M+H]⁺.

Synthesis of compound 5.5. Compound 5.5 was prepared from compound 5.4 following the procedure described in the synthesis of compound 1.11. The product was purified by flash column chromatography on silica gel (Combiflash®, 2.0% methanol in DCM). MS(ES): m/z 522.41 and 524.5 [M+H]⁺.

Synthesis of compound 5.6. Compound 5.6 was prepared from compound 5.5 following the procedure described in the synthesis of compound 1.12. The product was purified by flash column chromatography on silica gel (2.0% methanol in DCM). MS(ES): m/z 536.43 and 538.7 [M+H]⁺.

Synthesis of compound I-5. Compound I-5 was prepared from compound 5.6 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (50% ethyl acetate in hexane). MS(ES): m/z 538.63 [M+H]⁺. ¹H NMR (DMSO-d₆, 400 MHz): δ 8.40 (s, 1H), 8.16 (s, 1H), 8.12 (d, J=5.2 Hz, 1H), 7.83 (s, 1H), 7.80 (s, 1H), 7.41 (s, 1H), 7.24 (d, 1H), 6.97 (s, 1H), 4.16 (s, 3H), 3.65 (s, 3H), 3.65-3.50 (m, 1H), 2.71 (s, 6H), 2.29-2.24 (m, 1H), 2.11-2.00 (m, 1H), 1.52 (s, 1H), 1.33 (m, 1H), 1.23 (s, 3H), 1.23-1.14 (m, 2H), 0.91 (m, 1H).

### Example I-6: methyl ((1R,3S)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

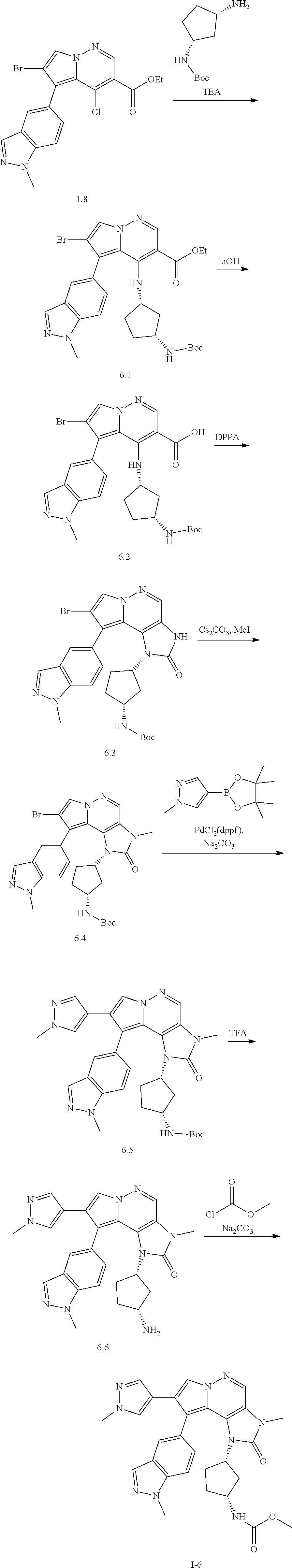

Synthesis of compound 6.1. Compound 6.1 was prepared from compound 1.8 and tert-butyl ((1R,3S)-3-aminocyclopentyl)carbamate following the procedure described in the synthesis of compound 1.9. The product was purified by flash column chromatography on silica gel (Combiflash®, 52% ethyl acetate in hexane). MS(ES): m/z 598.2 [M+H]⁺.

Synthesis of compound 6.2. Compound 6.2 was prepared from compound 6.1 following the procedure described in the synthesis of compound 1.10. The product was triturated with pentane, dried under reduced pressure and used in the next step without purification. MS(ES): m/z 570.1 [M+H]⁺.

Synthesis of compound 6.3. Compound 6.3 was prepared from compound 6.2 following the procedure described in the synthesis of compound 1.11. The product was purified by flash column chromatography on silica gel (Combiflash®, 1.6% methanol in DCM). MS(ES): m/z 567.4 [M+H]⁺.

Synthesis of compound 6.4. Compound 6.4 was prepared from compound 6.3 following the procedure described in the synthesis of compound 1.12. The product was purified by flash column chromatography on silica gel (1.3% methanol in DCM). MS(ES): m/z 581.3 [M+H]⁺.

Synthesis of compound 6.5. Compound 6.5 was prepared from compound 6.4 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (Combiflash®, 0-3.1% methanol in DCM). MS(ES): m/z 582.2

Synthesis of compound 6.6. Compound 6.6 was prepared from compound 6.5 following the procedure described in the synthesis of compound 1.14. The product was triturated with pentane and dried under reduced pressure. It was used in the next step without purification. MS(ES): m/z 482.4 [M+H]⁺.

Synthesis of compound I-6. Compound I-6 was prepared from compound 6.6 following the procedure described in the synthesis of compound I-1. The product was purified by flash column chromatography on silica gel (Combiflash®, 0-2.6% methanol in DCM). MS(ES): m/z 540.4 [M+H]⁺. ¹H NMR (DMSO-d₆, 400 MHz): δ 8.41 (d, J=3.6 Hz, 1H), 8.17-8.08 (m, 2H), 7.79-7.77 (m, 2H), 7.41-7.39 (m, 1H), 7.26 (s, 1H), 7.16-7.04 (m, 2H), 4.14 (s, 3H), 3.67 (s, 3H), 3.46 (s, 3H), 3.41 (s, 3H), 2.87-2.56 (m, 1H), 1.99-1.86 (m, 2H), 1.49-1.44 (m, 1H), 1.34-1.30 (m, 1H), 1.05-1.02 (m, 1H), 0.89-0.87 (m, 1H), 0.44-0.39 (m, 1H).

### Example I-7: cyanomethyl ((1R,3R)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

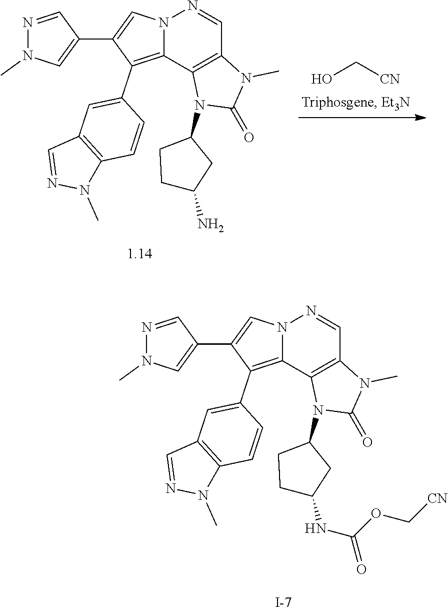

Synthesis of I-7. To a solution of 1.14 (0.075 g, 1.31 mmol, 1.0 equiv) and triethylamine (0.91 mL, 6.57 mmol, 5.0 equiv) in DCM (3 mL) at 0° C. was added a solution of triphosgene (0.136 g, 0.46 mmol, 0.35 equiv) in DCM. The reaction mixture was stirred at 0° C. for 20 min. It was poured into ice-water and extracted with ethyl acetate. The combined organic layers were washed with brine, dried over anhydrous sodium sulfate, filtered and concentrated under reduced pressure. The residue was purified by silica gel flash column chromatography on silica gel (5.8% methanol in DCM) to afford I-7 (0.018 g, 18.6%). MS(ES): m/z 565.45 [M+H]⁺. ¹H-NMR (400 MHz, DMSO-d₆): δ 8.32 (d, J=2.0 Hz, 1H), 8.08 (d, J=2.4 Hz, 1H), 8.04 (s, 1H), 7.75-7.72 (m, 2H), 7.41-7.39 (m, 1H), 7.23 (s, 1H), 7.01 (s, 1H), 6.89 (m, 0.5H), 6.59 (m, 0.5H), 4.80 (s, 2H), 4.13 (s, 3H), 3.97-3.75 (m, 1H), 3.61 (s, 3H), 3.36 (s, 3H), 2.07-2.05 (m, 1H), 1.83-1.64 (m, 2H), 0.83-1.64 (m, 2H), 0.46-0.08 (m, 1H).

### Example I-8: methyl ((1s,3s)-3-(3-methyl-9-(1-(methyl-d₃)-1H-indazol-5-yl)-8-(1-(methyl-d₃)-1H-pyrazol-4-yl)-2-oxo-2,3-dihydro-1H-imidazo[4,5-d]pyrrolo[1,2-b]pyridazin-1-yl)cyclobutyl)carbamate

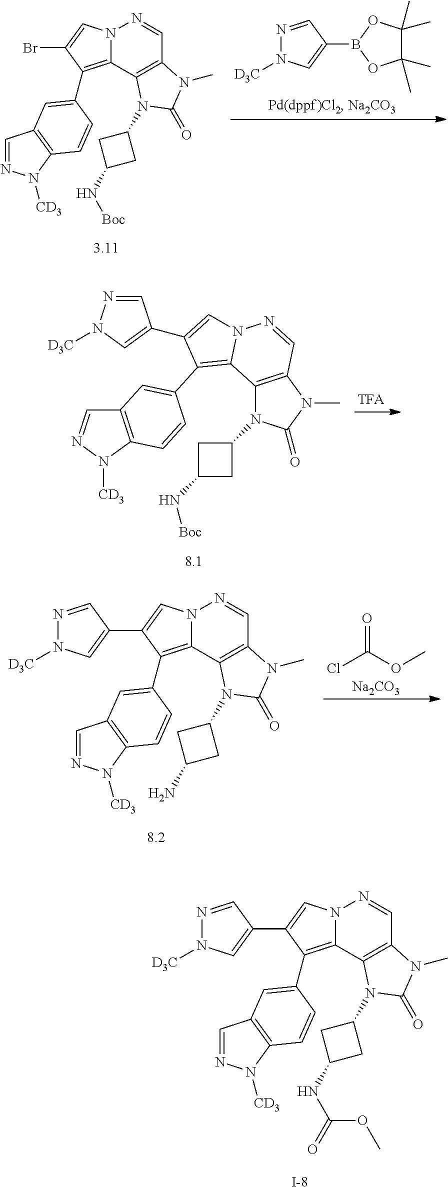

Synthesis of compound 8.1. Compound 8.1 was prepared from compound 3.11 and 1-(methyl-d₃)-4-(4,4,5,5-tetramethyl-1,3,2-dioxaborolan-2-yl)-1H-pyrazole following the procedure described in the synthesis of compound 1.13. The product was purified by flash column chromatography on silica gel (Combiflash®, 2.2% methanol in DCM). MS(ES): m/z 574 [M+H]⁺.

Synthesis of compound 8.2. Compound 8.2 was prepared from compound 8.1 following the procedure described in the synthesis of compound 1.14. The product was triturated with n-pentane and dried under reduced pressure. It was used in the next step without purification. MS(ES): m/z 474 [M+H]⁺.

Synthesis of I-8. Compound I-8 was prepared from compound 8.2 following the procedure described in the synthesis of compound I-1. The product was purified by flash column chromatography on silica gel (Combiflash®, 3.2% methanol in DCM). MS(ES): m/z 532.1 [M+H]⁺, ¹H NMR (DMSO-d₆, 400 MHz): δ 8.39 (s, 1H), 8.15 (s, 1H), 8.09 (s, 1H), 7.83 (d, J=8.8 Hz, 1H), 7.76 (s, 1H), 7.42-7.40 (m, 1H), 7.28 (m, 2H), 7.09 (s, 1H), 3.43 (s, 3H), 3.16-3.11 (m, 2H), 2.66-2.59 (m, 2H), 2.47 (s, 3H), 1.76-1.74 (m, 1H), 1.36-1.33 (m, 1H).

### Example I-9: 3-((1S,3S)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)oxazolidin-2-one

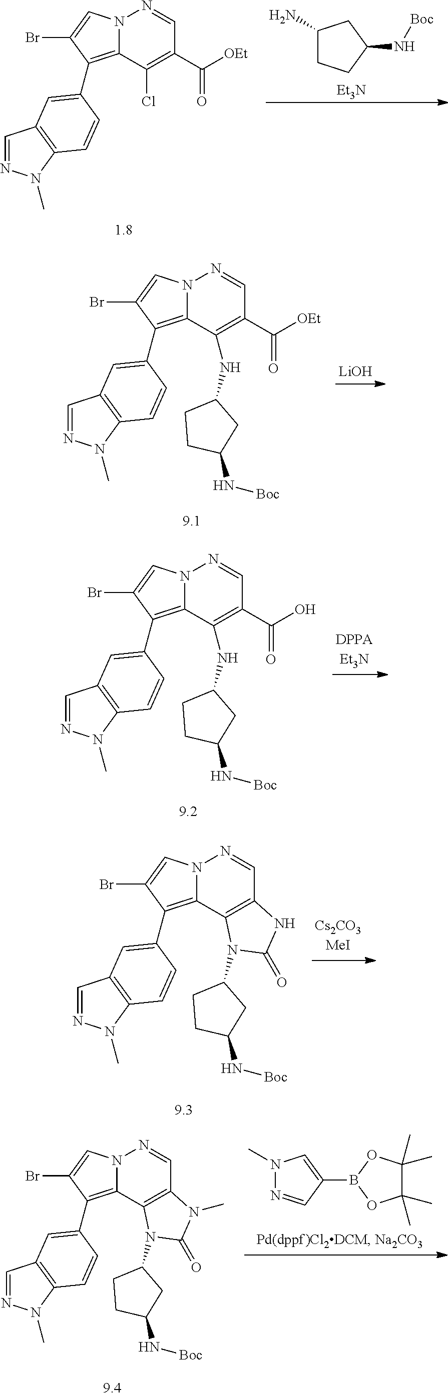

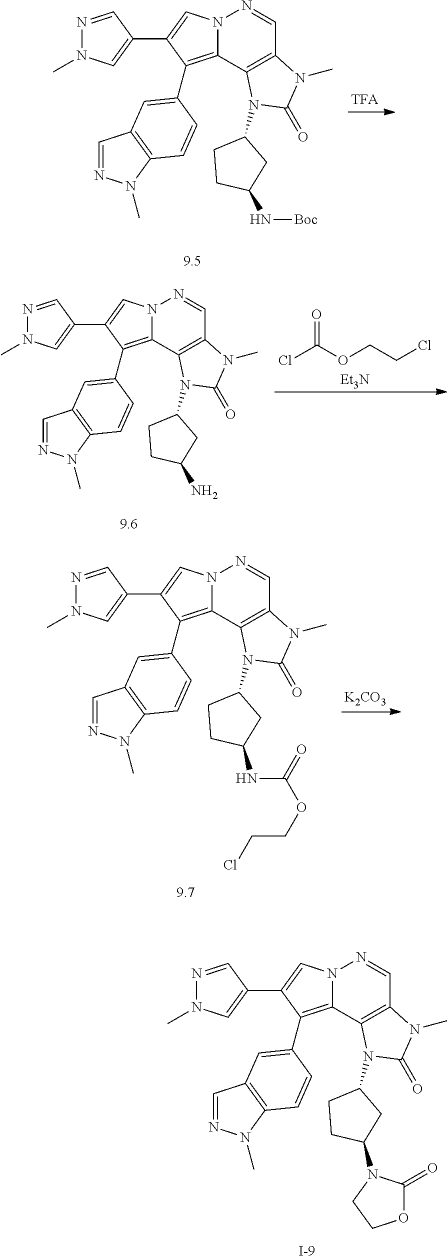

Synthesis of compound 9.1. Compound 9.1 was prepared from compound 1.8 and tert-butyl ((1S,3S)-3-aminocyclopentyl)carbamate following the procedure described in the synthesis of compound 1.9. The product was purified by flash column chromatography on silica gel (Combiflash®, 50% ethyl acetate in hexane). MS(ES): m/z 597.1 and 599.1 [M+H]⁺.

Synthesis of compound 9.2. Compound 9.2 was prepared from compound 9.1 following the procedure described in the synthesis of compound 1.10. The product was triturated with pentane, dried under reduced pressure and used in the next step without purification. MS(ES): m/z 569.4 and 571.4 [M+H]⁺.

Synthesis of compound 9.3. Compound 9.3 was prepared from compound 9.2 following the procedure described in the synthesis of compound 1.11. The product was purified by flash column chromatography on silica gel (Combiflash®, 1.8% methanol in DCM). MS(ES): m/z 566.5 and 568.5 [M+H]⁺.

Synthesis of compound 9.4. Compound 9.4 was prepared from compound 9.3 following the procedure described in the synthesis of compound 1.12. The product was purified by flash column chromatography on silica gel (1.4% methanol in DCM). MS(ES): m/z 580.2 and 582.2 [M+H]⁺.

Synthesis of compound 9.5. Compound 9.5 was prepared from compound 9.4 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (2.7% methanol in DCM). MS(ES): m/z 582.2 [M+H]⁺.

Synthesis of compound 9.6. Compound 9.6 was prepared from compound 9.5 following the procedure described in the synthesis of compound 1.14. The product was used without purification. MS(ES): m/z 482.30 [M+H]⁺.

Synthesis of compound 9.7. To a solution of 9.6 (0.16 g, 0.332 mmol, 1.0 equiv) and triethylamine (0.14 mL, 0.997 mmol, 3.0 equiv) in THF (4 mL) at 0° C. was added 2-chloroethyl carbonochloridate (0.095 g, 0.664 mmol, 2.0 equiv) dropwise and stirred at the same temperature for 10 min. It was transferred in water and extracted with ethyl acetate. The combined organic layers were washed with brine, dried over anhydrous sodium sulfate, filtered and concentrated under reduced pressure. The residue was purified by flash column chromatography on silica gel (Combiflash®, 3.2% methanol in DCM). MS(ES): m/z 589.3 [M+H]⁺.

Synthesis of I-9. A solution of 9.7 (0.11 g, 0.18 mmol, 1.0 equiv) and potassium carbonate (0.077 g, 0.56 mmol, 3 equiv) in dimethyl sulfoxide (3 mL) was stirred at 75° C. for 1 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 4.1% methanol in DCM) to afford I-9 (0.034 g, 33%). MS(ES): m/z 552.4 [M+H]⁺. ¹H-NMR (400 MHz, DMSO-d₆): δ 8.34 (d, J=2.4 Hz, 1H), 8.09 (d, J=2.8 Hz, 2H), 7.80-7.78 (m, 2H), 7.43 (bs, 1H), 7.24 (s, 1H), 7.02 (s, 1H), 4.19-4.15 (m, 6H), 3.67 (s, 3H), 3.32 (s, 3H), 2.54-2.50 (m, 2H), 2.08-1.89 (m, 3H), 1.61 (bs, 1H), 1.47-1.32 (m, 2H), 0.72-0.15 (m, 1H).

### Example I-10: 3-((1R,3S)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)oxazolidin-2-one

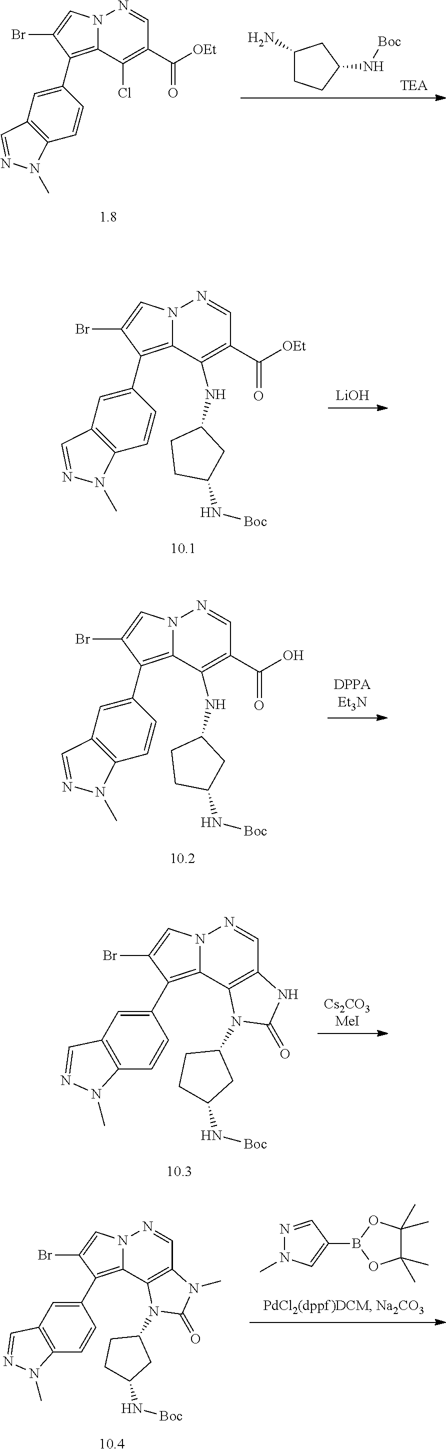

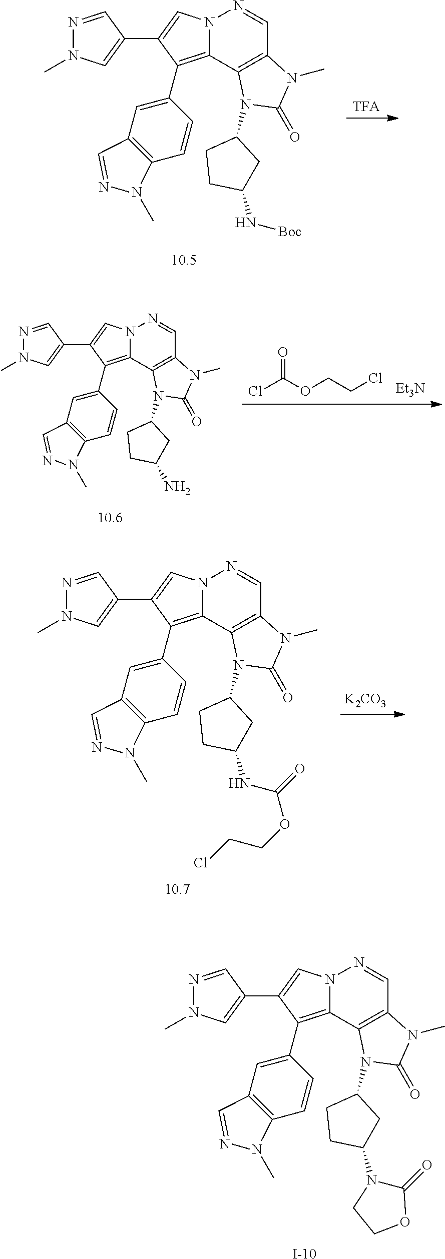

Synthesis of compound 10.1. Compound 10.1 was prepared from compound 1.8 and tert-butyl ((1R,3S)-3-aminocyclopentyl)carbamate following the procedure described in the synthesis of compound 1.9. The product was purified by flash column chromatography on silica gel (Combiflash®, 50% ethyl acetate in hexane). MS(ES): m/z 598.1 and 600.1 [M+H]⁺.

Synthesis of compound 10.2. Compound 10.2 was prepared from compound 10.1 following the procedure described in the synthesis of compound 1.10. The product was triturated with pentane, dried under reduced pressure and used in the next step without purification. MS(ES): m/z 570.4 572.4 [M+H]⁺.

Synthesis of compound 10.3. Compound 10.3 was prepared from compound 10.2 following the procedure described in the synthesis of compound 1.11. The product was purified by flash column chromatography on silica gel (Combiflash®, 1.8% methanol in DCM). MS(ES): m/z 567.5 and 569.5 [M+H]⁺.

Synthesis of compound 10.4. Compound 10.4 was prepared from compound 10.3 following the procedure described in the synthesis of compound 1.12. The product was purified by flash column chromatography on silica gel (1.4% methanol in DCM). MS(ES): m/z 582.2 and 584.2 [M+H]⁺.

Synthesis of compound 10.5. Compound 10.5 was prepared from compound 10.4 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (70% ethyl acetate in hexane). MS(ES): m/z 582 [M+H]⁺.

Synthesis of compound 10.6. Compound 10.6 was prepared from compound 10.5 following the procedure described in the synthesis of compound 1.14. The product was used without purification. MS(ES): m/z 482.30 [M+H]⁺.

Synthesis of compound 10.7. Compound 10.7 was prepared from compound 10.6 following the procedure described in the synthesis of compound 9.7. The product was purified by flash column chromatography on silica gel (Combiflash®, 3.2% methanol in DCM). MS(ES): m/z 588.3 [M+H]⁺.

Synthesis of I-10. Compound I-10 was prepared from compound 10.7 following the procedure described in the synthesis of compound I-9. The product was purified by flash column chromatography on silica gel (Combiflash®, 4.1% methanol in DCM). MS(ES): m/z 552.4 [M+H]⁺, ¹H NMR (DMSO-d₆, 400 MHz): δ 8.33 (d, J=2.8 Hz, 1H), 8.09 (s, 1H), 8.06 (s, 1H), 7.78-7.74 (m, 2H), 7.42-7.40 (m, 1H), 7.24 (s, 1H), 7.05 (s, 1H), 4.17-4.14 (m, 6H), 3.67 (s, 3H), 3.36-3.35 (m, 6H), 2.01 (m, 1H), 1.57 (m, 1H), 1.43 (m, 3H), 1.38 (m, 1H).

### Example I-11: tetrahydro-2H-pyran-4-yl ((1R,3S)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

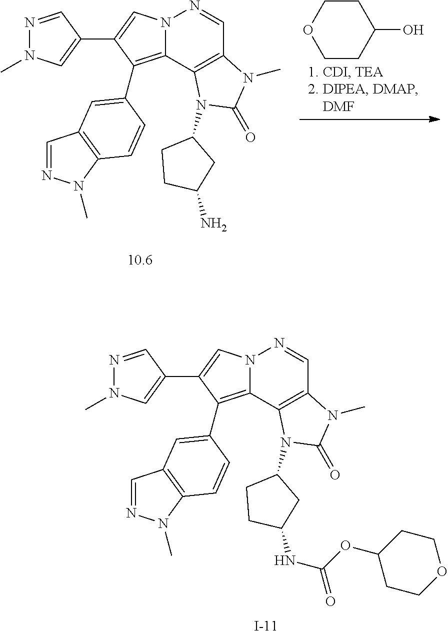

Synthesis of compound I-11. To a solution of tetrahydro-2H-pyran-4-ol (0.100 g, 0.980 mmol, 1.0 equiv) in DCM (5 mL) was added 1,1-carbonyldiimidazol (0.238 g, 1.47 mmol, 1.5 equiv) and triethylamine (0.148 g, 1.47 mmol, 1.0 equiv) at 0° C. The reaction mixture was stirred at room temperature for 2 h. It was quenched with water and extracted with DCM. The combined organic layers were washed with brine, dried over anhydrous sodium sulfate, filtered and concentrated under reduced pressure. The residue was added to a solution of 10.6 (0.100 g, 0.207 mmol, 1.0 equiv), DIPEA (0.200 g, 0.621 mmol, 3.0 equiv) and DMAP (2.5 g, 0.020 mmol, 0.1 equiv) in DMF. The reaction mixture was stirred at 65° C. for 16 h. It was quenched with water and extracted with ethyl acetate. The combined organic layers were washed with brine, dried over anhydrous sodium sulfate, filtered and concentrated under reduced pressure to afford material purified by flash column chromatography on silica gel (0-3.2% DCM in Methanol) to afford I-11 (21 mg, 17%). MS(ES): m/z 610 [M+H]⁺, ¹H NMR (DMSO-d₆, 400 MHz): δ 8.41-8.40 (d, J=8.8 Hz, 1H), 8.16 (s, 1H), 8.075 (s, 1H), 17.78 (m, 2H), 7.40-7.38 (d, J=8.8 Hz, 1H), 7.24 (s, 1H), 7.12-7.10 (m, 1H), 7.05-7.02 (m, 1H) 4.63 (bs, 1H), 4.14-4.12 (m, 4H), 3.76-3.70 (m, 3H), 3.65 (s, 3H), 3.32 (s, 3H), 1.93-1.90 (m, 1H), 1.88-1.81 (m, 3H), 1.53-1.45 (m, 4H), 1.33-1.23 (m, 3H), 1.28-1.14 (m, 1H).

### Example I-12: methyl methyl((1R,3S)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

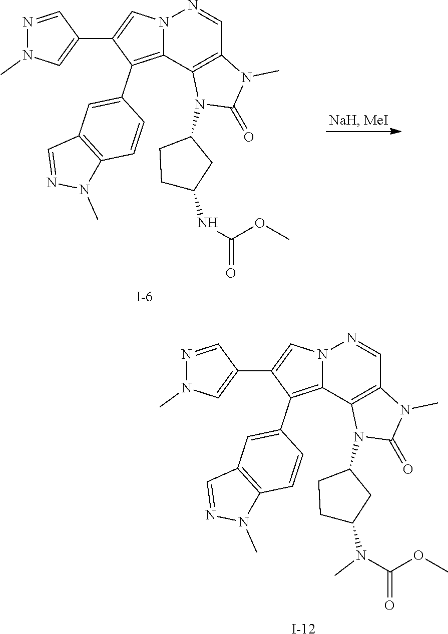

Synthesis of I-12. To a solution of I-6 (0.035 g, 0.065 mmol, 1.0 equiv) in DMF (4.0 mL) was added sodium hydride (60% dispersion in mineral oil, 0.013 g, 0.032 mmol, 5.0 equiv) at 0° C. in portions. The mixture was stirred for 15 min. To the mixture was added methyl iodide (0.018 g, 0.129 mmol, 2.0 equiv) at same temperature. The reaction mixture was stirred at room temperature for 15 min. It was quenched in water and extracted with ethyl acetate. The combined organic layers were washed with brine, dried over anhydrous sodium sulfate, filtered and concentrated under reduced pressure. The residue was purified by flash column chromatography on silica gel (Combiflash®, 0-2.2% methanol in DCM) to afford I-12 (0.018 g, 50%). MS(ES): m/z 554.4 [M+H]⁺, ¹H NMR (DMSO-d₆, 400 MHz): δ 8.39 (s, 1H), 8.16 (d, J=3.2 Hz, 1H), 8.09 (s, 1H), 7.82-7.78 (m, 2H), 7.42-7.39 (m, 1H), 7.25 (s, 1H), 7.06-7.01 (m, 1H), 4.17 (m, 3H), 3.66 (s, 3H), 3.54 (s, 3H), 3.38 (s, 3H), 2.59 (s, 3H), 2.33-2.27 (m, 1H), 2.20-2.18 (m, 1H), 2.18-1.99 (m, 2H), 1.51-1.48 (m, 1H), 1.33-1.29 (m, 1H), 1.01-0.99 (m, 1H), 0.48-0.42 (m, 1H).

### Example I-13: (1R,3S)—N-(3-(methoxymethyl)oxetan-3-yl)-N-methyl-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentane-1-carboxamide

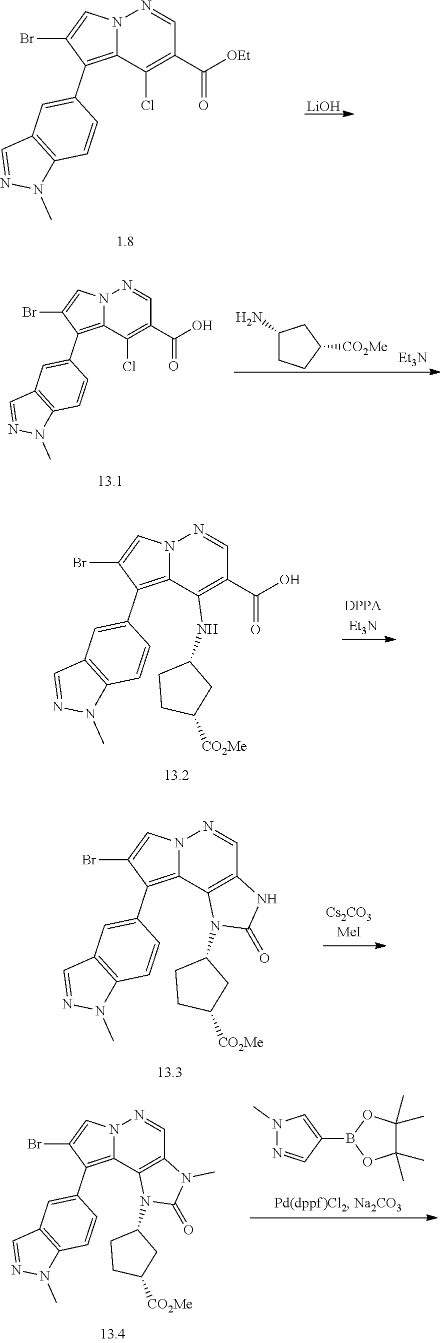

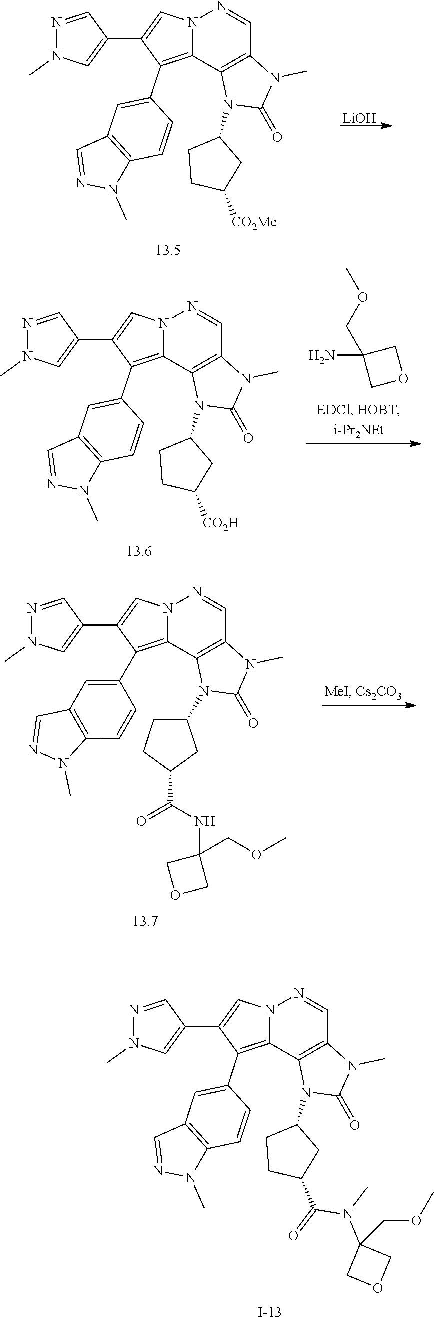

Synthesis of compound 13.1. To a solution of 1.8 (1.0 g, 2.31 mmol, 1.0 equiv) in a mixture of THF:MeOH:H₂O (2:2:1, 13 mL) was added LiOH·H₂O (1.93 g, 46.1 mmol, 20.0 equiv) at room temperature. The reaction mixture was stirred at room temperature for 3 h. It was transferred into ice-water and adjusted pH ˜5 to 6 with 1N HCl and extracted with 10% methanol in DCM. The combined organic layers were washed with brine, dried over anhydrous sodium sulfate, filtered and concentrated under reduced pressure. The residue was triturated with pentane, dried under vacuum and used in the next step without purification to afford 13.1 (0.740 g, 79%). MS(ES): m/z 402.97 and 404.9 [M+H]⁺.

Synthesis of compound 13.2. Compound 13.2 was prepared from compound 13.1 and methyl (1R,3S)-3-aminocyclopentane-1-carboxylate following the procedure described in the synthesis of compound 1.9. The product was purified by flash column chromatography on silica gel (Combiflash®, 50% ethyl acetate in hexane). MS(ES): m/z 510.09 and 512.2 [M+H]⁺.

Synthesis of compound 13.3. Compound 13.3 was prepared from compound 13.2 following the procedure described in the synthesis of compound 1.11. The product was purified by flash column chromatography on silica gel (Combiflash®, 1.8% methanol in DCM). MS(ES): m/z 507.09 and 509.1 [M+H]⁺.

Synthesis of compound 13.4. Compound 13.4 was prepared from compound 13.3 following the procedure described in the synthesis of compound 1.12. The product was purified by flash column chromatography on silica gel (1.4% methanol in DCM). MS(ES): m/z 522.10 and 524.2 [M+H]⁺.

Synthesis of compound 13.5. Compound 13.5 was prepared from compound 13.4 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (1.5% methanol in DCM). MS(ES): m/z 524.23 [M+H]⁺.

Synthesis of compound 13.6. To a solution of 13.5 (0.380 g, 0.72 mmol, 1.0 equiv) in a mixture of THF:MeOH:H₂O (2:2:1, 33 mL) was added lithium hydroxide hydrate (0.608 g, 14.4 mmol, 20.0 equiv) at room temperature. The reaction mixture was stirred at room temperature for 3 h. It was transferred into ice-water and adjusted pH ˜5 to 6 with 1N HCl and extracted with 10% methanol in DCM. The combined organic layers were washed with brine, dried over anhydrous sodium sulfate, filtered and concentrated under reduced pressure. The residue was triturated with pentane, dried under vacuum and used in the next step without purification. afford 13.6 (0.260 g, 70%). MS(ES): m/z 511.21 [M+H]⁺.

Synthesis of compound 13.7. A solution of 13.6 (0.125 g, 0.244 mmol, 1.0 equiv), 1-hydroxybenzotriazole (0.036 g, 0.269 mmol, 1.1 equiv), 1-ethyl-3-(3-dimethylaminopropyl) carbodiimide hydrochloride (0.070 g, 0.367 mmol, 1.5 equiv) and N,N-diisopropylethylamine (0.21 mL, 1.22 mmol, 5.0 equiv) in DCM (30 mL) was stirred at room temperature for 15 min followed by addition of 3-(methoxymethyl)oxetan-3-amine (0.037 g, 0.31 mmol, 1.3 equiv) and stirred at room temperature for 16 h. It was transferred into ice-water, stirred and extracted with DCM. The combined organic layers were washed with brine, dried over anhydrous sodium sulfate, filtered and concentrated under reduced pressure. The residue was purified by flash column chromatography on silica gel (2.6% methanol in DCM) to afford 13.7 (0.063 g, 43%). MS (ES): m/z 610.2 [M+H]⁺.

Synthesis of I-13. To a solution of 13.7 (0.063 g, 0.105 mmol, 1.0 equiv) and cesium carbonate (0.051 g, 0.158 mmol, 1.5 equiv) in DMF (2 mL) was added methyl iodide (0.018 mg, 0.126 mmol, 1.2 equiv) at 0° C. The reaction mixture was stirred at room temperature for 15 min. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (1.4% methanol in DCM) to afford I-37 (0.018 g, 27%). MS(ES): m/z 624.30 [M+H]⁺, ¹H NMR (DMSO-d₆, 400 MHz): δ 8.40 (s, 1H), 8.17 (d, 2H), 7.81 (s, 2H), 7.43-7.14 (d, 1H), 7.28-7.26 (s, 1H), 7.04 (s, 1H), 4.19 (s, 3H), 4.04-4.00 (m, 2H), 3.71 (s, 3H), 3.47 (m, 2H), 3.28 (s, 6H), 2.62 (s, 6H), 2.08-1.97 (m, 2H), 1.81-1.50 (m, 3H), 0.92-0.73 (m, 2H).

### Example I-14: (1R,3S)—N-(cyanomethyl)-N-methyl-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentane-1-carboxamide

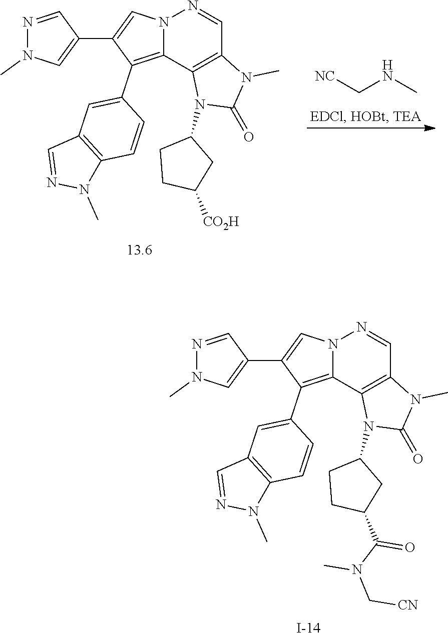

Synthesis of I-14. A solution of 13.6 (0.060 g, 0.117 mmol, 1.0 equiv), 1-ethyl-3-(3-dimethylaminopropyl) carbodiimide (0.027 g, 0.176 mmol, 1.5 equiv) and triethylamine (0.035 g, 0.352 mmol, 3.0 equiv) in DCM (3.0 mL) at 0° C. was added hydroxybenzotriazole (0.023 g, 0.176 mmol, 1.5 equiv) and 2-(methylamino) acetonitrile (0.041 g, 0.588 mmol, 5.0 equiv). The reaction mixture was stirred for 3 h. It was quenched in water and extracted with ethyl acetate. The combined organic layers were washed with brine, dried over anhydrous sodium sulfate, filtered and concentrated under reduced pressure. The residue was purified by flash column chromatography on silica gel (Combiflash®, 0-5% methanol in DCM) to afford I-14 (0.021 g, 32%). MS(ES): m/z 563.64 [M+H]⁺, ¹H NMR (DMSO-d₆, 400 MHz): δ 8.39 (s, 1H), 8.16 (d, 1H), 8.13 (s, 1H), 7.86 (s, 1H), 7.82 (s, 1H), 7.42 (s, 1H), 7.25 (s, 1H), 6.97 (s, 1H), 4.30-4.27 (m, 2H), 4.13 (s, 3H), 3.69 (s, 3H), 2.82-2.79 (m, 1H), 2.66-2.61 (m, 1H), 2.31-2.29 (m, 1H), 2.02-1.98 (m, 1H), 1.58-1.54 (m, 1H), 1.33-1.31 (m, 2H), 1.15 (s, 6H), 0.85-0.82 (m, 1H).

### Example I-15: methyl ((1R,3R)-3-(8-(2,3-dihydropyrazolo[5,1-b]oxazol-7-yl)-3-methyl-9-(1-methyl-1H-indazol-5-yl)-2-oxo-2,3-dihydro-1H-imidazo[4,5-d]pyrrolo[1,2-b]pyridazin-1-yl)cyclopentyl)carbamate

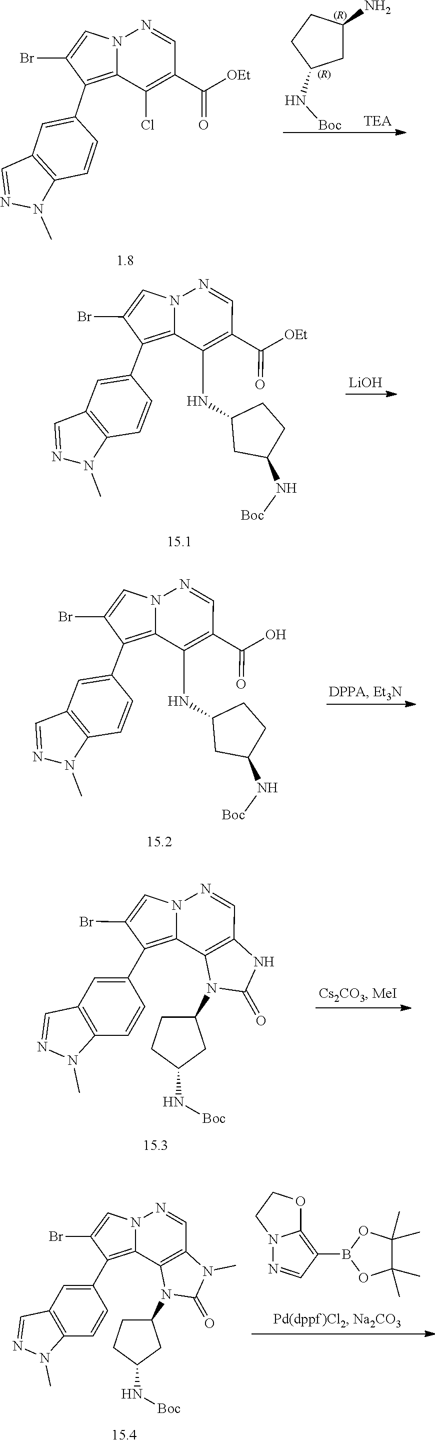

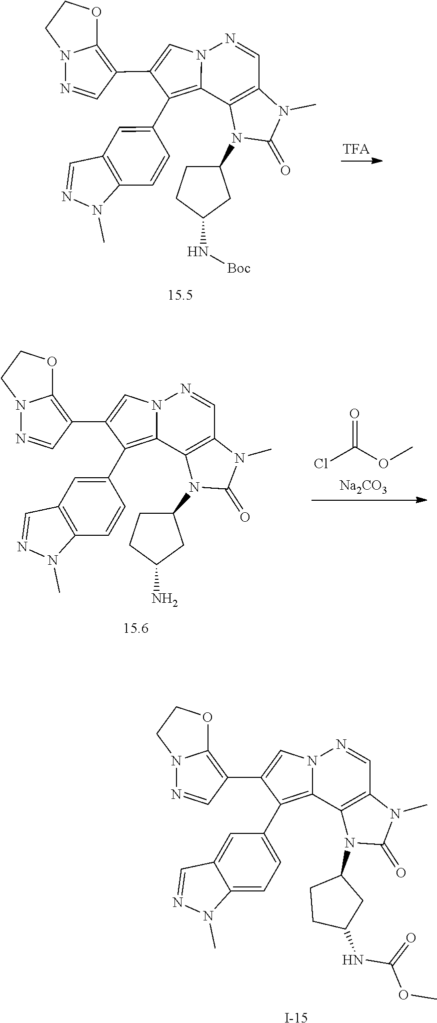

Synthesis of compound 15.1. Compound 15.1 was prepared from compound 1.8 and tert-butyl ((1R,3R)-3-aminocyclopentyl)carbamate following the procedure described in the synthesis of compound 1.9. The product was purified by flash column chromatography on silica gel (Combiflash®, 53% ethyl acetate in hexane). MS(ES): m/z 597.5 and 599.5 [M+H]⁺.

Synthesis of compound 15.2. Compound 15.2 was prepared from compound 15.1 following the procedure described in the synthesis of compound 1.10. The product was triturated with pentane, dried under reduced pressure and used in the next step without purification. MS(ES): m/z 568.3 and 570.3 [M+H]⁺.

Synthesis of compound 15.3. Compound 15.3 was prepared from compound 15.2 following the procedure described in the synthesis of compound 1.11. The product was purified by flash column chromatography on silica gel (Combiflash®, 1.8% methanol in DCM). MS(ES): m/z 566.1 and 567.1 [M+H]⁺.

Synthesis of compound 15.4. Compound 15.4 was prepared from compound 15.3 following the procedure described in the synthesis of compound 1.12. The product was purified by flash column chromatography on silica gel (1.2% methanol in DCM). MS(ES): m/z 579.1 and 581.2 [M+H]⁺.

Synthesis of compound 15.5. Compound 15.5 was prepared from compound 15.4 and 7-(4,4,5,5-tetramethyl-1,3,2-dioxaborolan-2-yl)-2,3-dihydropyrazolo[5,1-b]oxazole following the procedure described in the synthesis of compound 1.13. The product was purified by flash column chromatography on silica gel (Combiflash®, 0-2.8% methanol in DCM). MS(ES): m/z 610.2 [M+H]⁺.

Synthesis of compound 15.6. Compound 15.6 was prepared from compound 15.5 following the procedure described in the synthesis of compound 1.14. The product was triturated with pentane and dried under reduced pressure. It was used in the next step without purification. MS(ES): m/z 510.1 [M+H]⁺.

Synthesis of I-15. Compound I-15 was prepared from compound 15.6 following the procedure described in the synthesis of compound I-1. The product was purified by flash column chromatography on silica gel (Combiflash®, 0-2.4% methanol in DCM). MS(ES): m/z 568.4 [M+H]⁺, ¹H NMR (DMSO-d₆, 400 MHz): δ 8.37 (s, 1H), 8.06 (d, J=7.2 Hz, 1H), 7.81 (s, 1H), 7.75 (d, J=6.8 Hz, 2H), 7.40 (t, J=8.0 Hz, 1H), 6.64 (s, 0.6H), 6.26 (s, 0.5H), 6.15 (s, 0.3H), 5.13-5.11 (m, 2H), 4.22-4.18 (m, 2H), 4.04 (s, 3H), 3.87-3.85 (m, 1H), 3.54-3.49 (m, 1H), 3.44 (s, 3H), 3.31 (s, 3H), 2.00-1.99 (m, 1H), 1.83-1.78 (m, 1H), 1.68-1.51 (m, 1H), 1.11-1.05 (m, 2H), 0.48-0.41 (m, 0.4H), −0.13-0.17 (m, 0.6H).

### Example I-16: methyl ((1R,3R)-3-(8-(5,6-dihydro-4H-pyrrolo[1,2-b]pyrazol-3-yl)-3-methyl-9-(1-methyl-1H-indazol-5-yl)-2-oxo-2,3-dihydro-1H-imidazo[4,5-d]pyrrolo[1,2-b]pyridazin-1-yl)cyclopentyl)carbamate

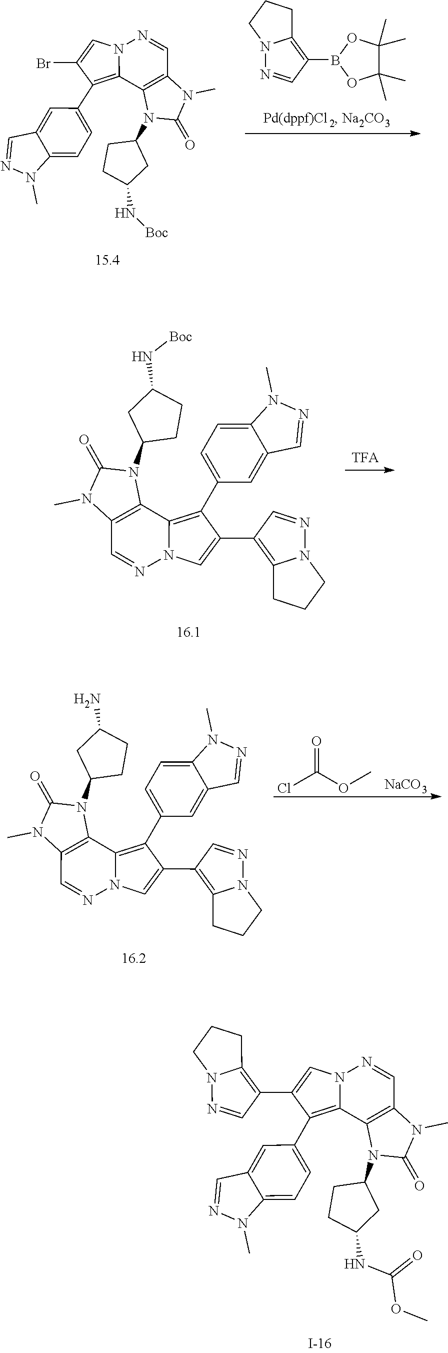

Synthesis of compound 16.1. Compound 16.1 was prepared from compound 15.4 and 3-(4,4,5,5-tetramethyl-1,3,2-dioxaborolan-2-yl)-5,6-dihydro-4H-pyrrolo[1,2-b]pyrazole following the procedure described in the synthesis of compound 1.13. The product was purified by flash column chromatography on silica gel (35% ethyl acetate in hexane). MS(ES): m/z 608.7 [M+H]⁺.

Synthesis of compound 16.2. Compound 16.2 was prepared from compound 16.1 following the procedure described in the synthesis of compound 1.14. The product was triturated with pentane and dried under reduced pressure. It was used in the next step without purification. MS(ES): m/z 507.6 [M+H]⁺.

Synthesis of I-16. Compound I-16 was prepared from compound 16.2 following the procedure described in the synthesis of compound I-1. The product was purified by flash column chromatography on silica gel (Combiflash®, 0-2.6% methanol in DCM). MS(ES): m/z 566.4 [M+H]⁺, ¹H NMR (DMSO-d₆, 400 MHz): δ 8.39 (s, 1H), 8.06-8.04 (d, 1H), 7.97-7.96 (s, 1H), 7.76-7.74 (s, 2H), 7.40-7.38 (d, J=8.8 Hz, 1H), 6.67-6.66 (d, 0.5H), 6.57 (s, 1H), 6.31 (s, 0.5H), 4.12 (s, 3H), 3.97-3.85 (t, 2H), 3.52-3.46 (m, 1H), 3.44-3.38 (m, 4H), 3.37-3.30 (m, 4H), 3.27-2.70 (m, 3H), 2.10-1.92 (m, 1H), 1.78-1.65 (m, 1H), 1.62-1.42 (m, 1H), 1.41-1.29-(m, 2H), 0.48-0.41 (m, 0.4H), −0.13-0.17 (m, 0.6H).

### Example I-17: 2-methoxy-N-methyl-N-((1S,3S)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)acetamide

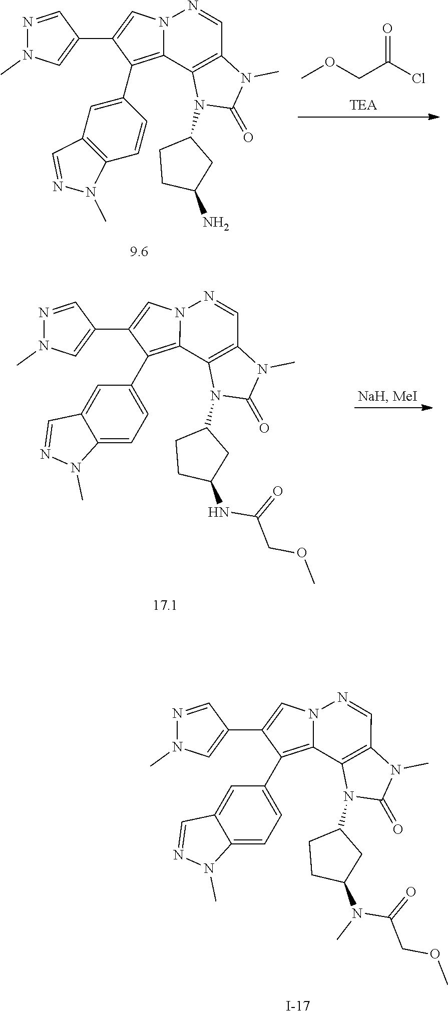

Synthesis of compound 17.1. A solution of 9.6 (0.1 g, 0.207 mmol, 1.0 equiv), 2-methoxyacetyl chloride (0.045 g, 0.415 mmol, 2.0 equiv) and triethylamine (0.063 g, 0.623 mmol, 3.0 equiv) in THF (5 mL) was stirred at room temperature for 1 h. It was transferred into water and extracted with ethyl acetate. The combined organic layers were washed with brine, dried over anhydrous sodium sulfate, filtered and concentrated under reduced pressure. The residue was purified by flash column chromatography on silica gel (Combiflash®, 2.8% methanol in DCM) to afford 17.1 (0.07 g, 61%). MS(ES): m/z 554.4 [M+H]⁺.

Synthesis of I-17. To a solution of 17.1 (0.07 g, 0.126 mmol, 1.0 equiv) in DMF (4 mL) was added sodium hydride (in 60% mineral oil, 0.025 g, 0.63 mmol, 5.0 equiv) at 0° C. The reaction mixture was stirred at room temperature for 1 h. Methyl iodide (0.071 g, 0.50 mmol, 4.0 equiv) was added to the reaction mixture and stirred for 1 h at room temperature. It was poured into ice-water and extracted with ethyl acetate. The combined organic layers were washed with brine, dried over anhydrous sodium sulfate, filtered and concentrated under reduced pressure. The residue was purified by flash column chromatography on silica gel (3.5% methanol in DCM) to afford I-17 (0.026 g, 36%). MS(ES): m/z 568.43 [M+H]⁺, ¹H-NMR (400 MHz, DMSO-d₆): δ 8.41 (s, 1H), 8.22-8.12 (m, 2H), 7.87-7.81 (m, 2H), 7.47 (dd, J=8.00, 29.6 Hz, 1H), 7.26 (s, 1H), 7.05-6.99 (m, 1H), 4.19-3.89 (m, 6H), 3.66 (s, 3H), 3.51 (bs, 1H), 3.36 (s, 3H), 3.32 (s, 3H), 2.22-2.18 (m, 1H), 2.06-1.87 (m, 4H), 1.39-1.24 (m, 3H), 0.76 (bs, 1H).

### Example I-18: N-ethyl-2-hydroxy-N-((1S,3S)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)acetamide

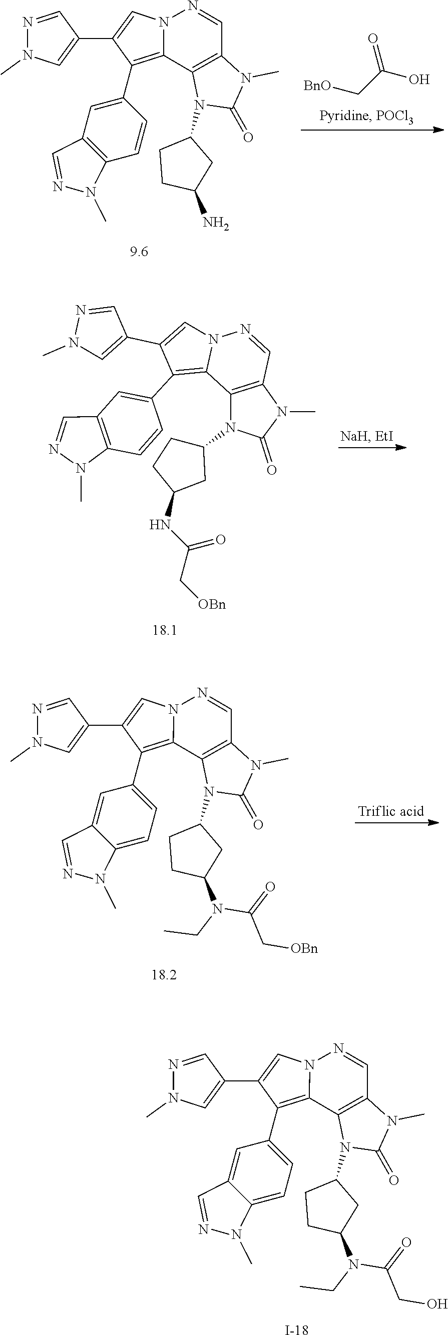

Synthesis of compound 18.1. To a solution of 9.6 (0.160 g, 0.332 mmol, 1.0 equiv), pyridine (0.078 g, 0.997 mmol, 3.0 equiv) and 2-(benzyloxy)acetic acid (0.066 g, 0.399 mmol, 1.2 equiv) in DCM (5.0 mL) at 0° C. was added phosphorus oxychloride (0.076 g, 0.498 mmol, 1.5 equiv) dropwise and stirred for 10 min. It was quenched in saturated sodium bicarbonate solution and extracted with DCM. The combined organic layers were washed with brine, dried over anhydrous sodium sulfate, filtered and concentrated under reduced pressure. The residue was purified by flash column chromatography on silica gel (Combiflash®, 2.8% methanol in DCM) to afford 18.1 (0.090 g, 43%). MS(ES): m/z 630.6 [M+H]⁺.

Synthesis of compound 18.2. To a solution of 18.1 (0.09 g, 0.143 mmol, 1.0 equiv) in DMF (4 mL) was added sodium hydride (60% mineral oil) (0.011 g, 0.286 mmol, 2.0 equiv) at 0° C. The reaction mixture was stirred at same temperature for 15 min. To the mixture was added ethyl iodide (0.029, 0.185 mmol, 1.3 equiv) and stirred at room temperature for 1 h. It was transferred into water and extracted with ethyl acetate. The combined organic layers were washed with brine, dried over anhydrous sodium sulfate, filtered and concentrated under reduced pressure. The residue was purified by flash column chromatography on silica gel (2.4% methanol in DCM) to afford 18.2 (0.05 g, 53%). MS(ES): m/z 658.8 [M+H]⁺.

Synthesis of I-18. To a solution of 18.2 (0.05 g, 0.076 mmol, 1.0 equiv) in DCM (3 mL) was added trifluoromethanesulfonic acid (0.5 mL) at 0° C. The reaction mixture was stirred for 15 min. It was transferred into a saturated sodium bicarbonate solution and extracted with DCM. The combined organic layers were washed with brine, dried over anhydrous sodium sulfate, filtered and concentrated under reduced pressure. The residue was purified by flash column chromatography on silica gel (Combiflash®, 4.5% methanol in DCM) to afford I-18 (0.015 g, 35%). MS(ES): m/z 568.42 [M+H]⁺, ¹H NMR (400 MHz, DMSO-d₆): δ 8.32 (s, 1H), 8.09 (d, J=4.0 Hz, 2H), 7.80-7.78 (m, 2H), 7.41 (bs, 1H), 7.24 (s, 1H), 7.00 (s, 1H), 4.42 (bs, 1H), 4.12 (s, 3H), 3.96 (bs, 2H), 3.66 (s, 3H), 3.41 (m, 2H), 3.36 (s, 3H), 2.03-1.84 (m, 2H), 1.58 (m, 1H), 1.43-1.25 (m, 2H), 1.11-1.07 (m, 3H), 0.77 (bs, 2H), 0.49-0.17 (m, 1H).

### Example I-19: methyl ((1R,3R)-3-(3-methyl-8-(1-methyl-1H-1,2,3-triazol-4-yl)-9-(1-methyl-1H-indazol-5-yl)-2-oxo-2,3-dihydro-1H-imidazo[4,5-d]pyrrolo[1,2-b]pyridazin-1-yl)cyclopentyl)carbamate

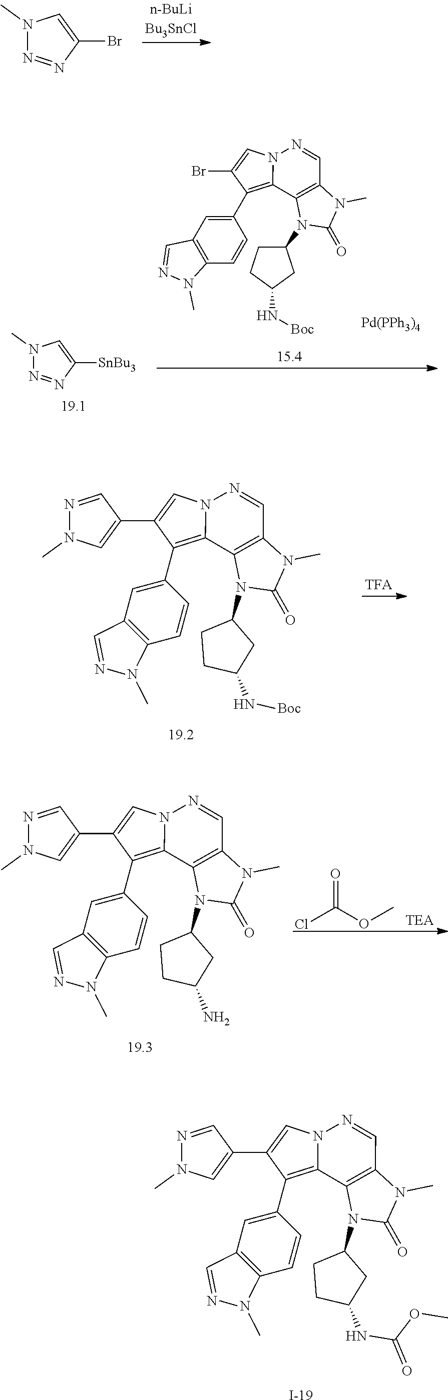

Synthesis of compound 19.1. To a solution of 4-bromo-1-methyl-1H-1,2,3-triazole (0.5 g, 3.08 mmol, 1.0 equiv) in diethyl ether (10 mL) was added n-butyl lithium (2.5M in hexane, 3.0 mL, 4.93 mmol, 1.6 equiv) dropwise at −78° C. and stirred for 20 min. Tributyl tin chloride (1.2 g, 3.85 mmol, 1.25 equiv) was added and the reaction mixture was stirred at 0° C. for 30 min. It was transferred into water and extracted with diethyl ether. The combined organic layers were washed with brine, dried over anhydrous sodium sulfate, filtered and concentrated under reduced pressure to afford 19.1 (0.500 g, 43.5%), which was used in the next step without purification.

Synthesis of compound 19.2. A solution of 15.4 (0.2 g, 0.34 mmol, 1.0 equiv) and 19.1 (0.64 g, 1.72 mmol, 5.0 equiv) in 1,4-dioxane (35 mL) was degassed by bubbling through a stream of argon for 10 min. Tetrakis (triphenylphosphine) palladium (0) (0.039 g, 0.034 mmol, 0.1 equiv) was added to the reaction mixture and purged with argon for 10 min. The mixture was stirred at 140° C. for 1 h. It was poured into water and extracted with ethyl acetate. The combined organic layers were washed with brine, dried over anhydrous sodium sulfate, filtered and concentrated under reduced pressure. The residue was purified by flash column chromatography on silica gel (2.2% methanol in DCM) to afford 19.2 (0.12 g, 60%). MS(ES): m/z 583.81 [M+H]⁺.

Synthesis of compound 19.3. Compound 19.3 was prepared from compound 19.2 following the procedure described in the synthesis of compound 1.14. The product was used in the next step without purification. MS(ES): m/z 483.3 [M+H]⁺.

Synthesis of I-19. Compound I-19 was prepared from compound 19.3 following the procedure described in the synthesis of compound I-1. The product was purified by silica gel flash column chromatography on silica gel (3.6% methanol in DCM). MS(ES): m/z 541.46 [M+H]⁺, ¹H NMR (DMSO-d₆, 400 MHz): δ 8.39 (d, J=2.8 Hz, 1H), 8.22 (d, J=2.8 Hz, 1H), 8.04 (s, 1H), 7.73-7.77 (m, 2H), 7.42-7.40 (m, 1H), 6.99 (bs, 1H), 6.11 (bs, 1H), 4.13 (s, 3H), 4.09-4.01 (m, 2H), 3.85 (s, 3H), 3.50 (s, 3H), 3.40 (s, 3H), 2.07-1.84 (m, 3H), 1.19-1.12 (m, 2H), 0.08 (bs, 1H).

### Example I-20-a and I-20-b: 2-((R)-1,4-dioxan-2-yl)-N-((1s,3S)-3-(3-methyl-9-(1-(methyl-d₃)-1H-indazol-5-yl)-8-(1-(methyl-d₃)-1H-pyrazol-4-yl)-2-oxo-2,3-dihydro-1H-imidazo[4,5-d]pyrrolo[1,2-b]pyridazin-1-yl)cyclobutyl)acetamide and 2-((S)-1,4-dioxan-2-yl)-N-((1s,3S)-3-(3-methyl-9-(1-(methyl-d₃)-1H-indazol-5-yl)-8-(1-(methyl-d₃)-1H-pyrazol-4-yl)-2-oxo-2,3-dihydro-1H-imidazo[4,5-d]pyrrolo[1,2-b]pyridazin-1-yl)cyclobutyl)acetamide

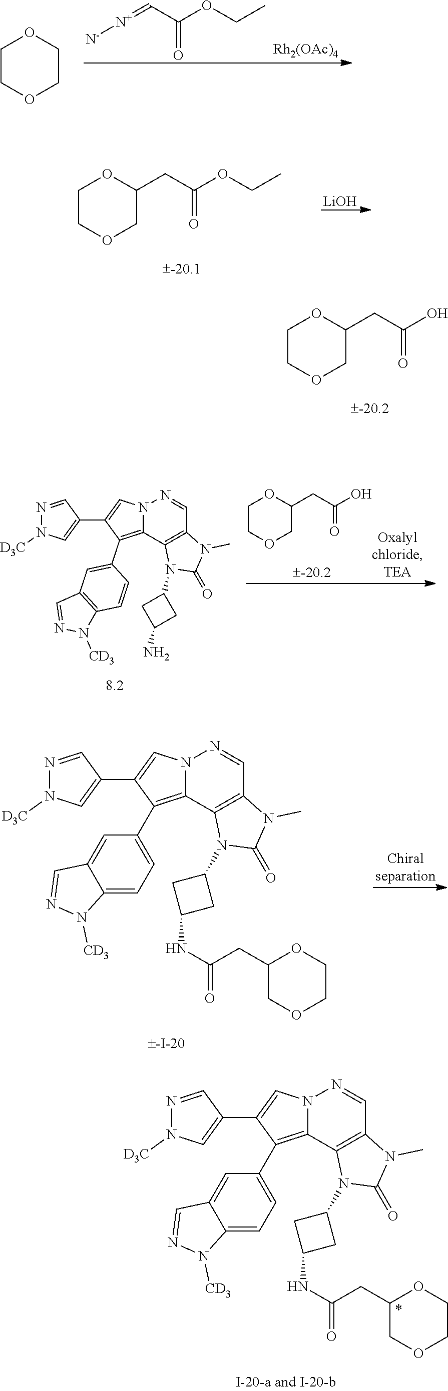

Synthesis of compound ±−20.1. A mixture of 1,4-dioxane (1.0 g, 11.363 mmol, 1.0 equiv) and dirhodium tetraacetate (0.002 mg) was stirred at 60° C. and was added a solution of ethyl 2-diazoacetate (0.08 g) in dioxane (10 mL) dropwise over a 2 h period. The reaction mixture was cooled to room temperature and filtered through a pad of alumina. The filtrate was concentrated under reduced pressure to remove excess of dioxane. The residue was purified by vacuum distillation to afford a colorless oil (0.630 g, 32%). MS(ES): m/z 175.20 [M+H]⁺.

Synthesis of compound ±−20.2. To a stirred solution of ±−20.1 (0.630 g, 3.62 mmol, 1.0 equiv) in a mixture of THF:MeOH:H₂O (2:2:1, 10 mL) was added lithium hydroxide hydrate (3.04 g, 72.413 mmol, 20.0 equiv) at room temperature. The reaction mixture was stirred for 3 h. It was transferred into ice-water and acidified with 1N HCl. The mixture was extracted with 10% MeOH in DCM. The combined organic layers were washed with brine, dried over anhydrous sodium sulfate, filtered and concentrated under reduced pressure. The residue was triturated with pentane, dried under vacuum and used in the next step without purification. afford ±−20.2 (0.390 g, 74%). MS(ES): m/z 147.14 [M+H]⁺.

Synthesis of compound ±−I-20. To a stirred solution of ±−20.2 (0.390 g, 2.671 mmol, 1.0 equiv) in DCM (5 mL) at 0° C. was added oxalyl chloride (0.508 g, 4.006 mmol, 1.5 equiv) dropwise and stirred for 15 min at room temperature. The reaction mixture was added to a stirred solution of 8.2 (0.290 g, 0.613 mmol, 1.0 equiv) and triethylamine (0.186 g, 1.839 mmol, 3.0 equiv) in THF (4 mL) at 0° C. and stirred for 20 min. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 0-5% methanol in DCM) to afford ±−I-20 (0.065 g, 18%). MS(ES): m/z 602.70 [M+H]⁺.

The racemate was separated by SFC (column: CHIRALCEL OX-H (250*4.6 mm, 5 μm); mobile phases: (A) CO₂, (B) 0.1% NH₃ in IPA: ACN (50:50); flow rate: 4 mL/min) to afford first eluting fraction (I-20-a 0.017 g, 26%): MS(ES): m/z 602.70 [M+H]⁺, ¹H NMR (DMSO-d₆, 400 MHz): δ 8.39 (s, 1H), 8.15 (s, 1H), 8.08 (s, 1H), 8.00-7.98 (d, J=7.2 Hz., 1H), 7.85-7.83 (d, J=8 Hz., 1H), 7.75 (s, 1H), 7.43-7.41 (d, J=8.4 Hz., 1H), 7.28 (s, 1H), 7.10 (s, 1H), 3.83-3.79 (m, 1H), 3.77-3.65 (m, 2H), 3.55 (m, 2H), 3.41 (m, 2H), 3.38 (s, 3H), 3.13-3.08 (m, 2H), 2.63-2.58 (m, 2H), 2.12 (m, 1H), 2.08 (m, 1H), 1.78 (m, 1H), 1.34 (m, 1H) and second eluting fraction (I-20-b 0.018 g, 28%): MS(ES): m/z 602.70 [M+H]⁺, ¹H NMR (DMSO-d₆, 400 MHz): δ 8.39 (s, 1H), 8.15 (s, 1H), 8.08 (s, 1H), 8.00-7.99 (d, J=7.2 Hz., 1H), 7.85-7.83 (d, J=8 Hz., 1H), 7.75 (s, 1H), 7.43-7.41 (d, J=8.4 Hz., 1H), 7.28 (s, 1H), 7.10 (s, 1H), 3.71 (m, 1H), 3.65-3.58 (m, 2H), 3.55 (m, 2H), 3.41 (m, 2H), 3.38 (s, 3H), 3.12-3.08 (m, 2H), 2.63-2.58 (m, 2H), 2.12 (m, 1H), 2.08 (m, 1H), 1.78 (m, 1H), 1.34 (m, 1H)

### Example I-21: (1s,3s)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclobutyl oxetan-3-ylcarbamate

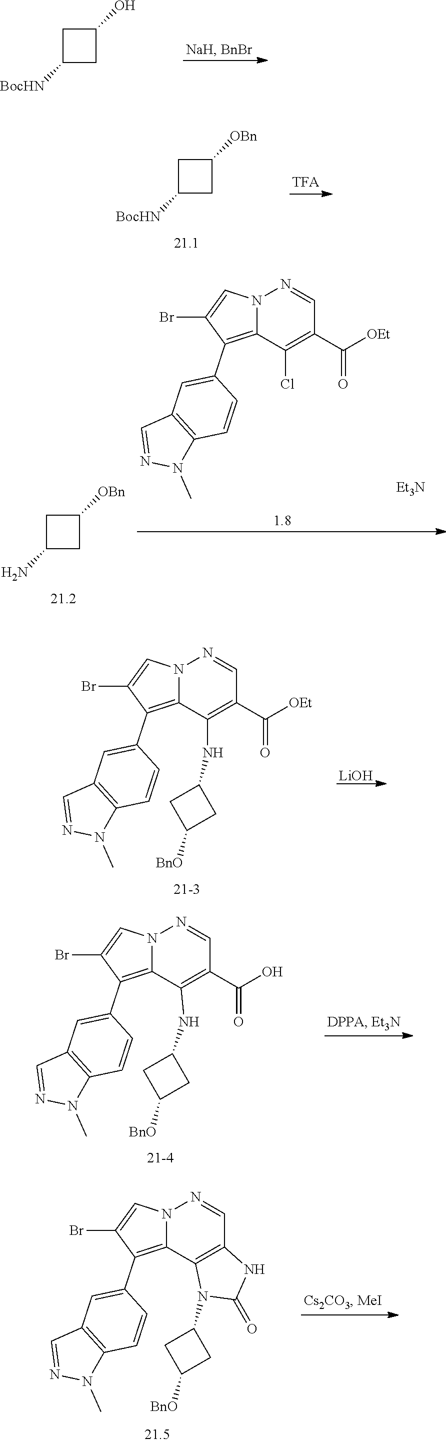

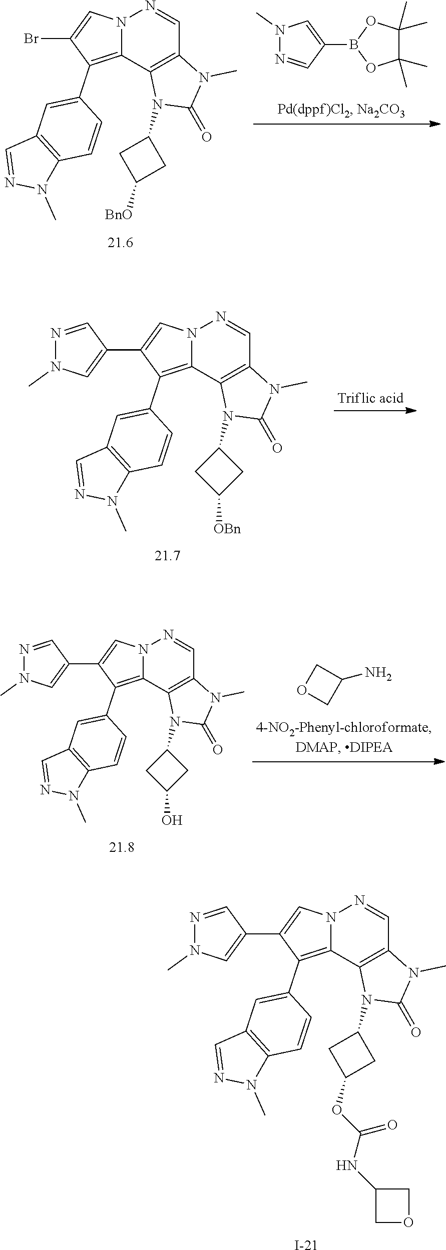

Synthesis of compound 21.1 To a solution of tert-butyl ((1s,3s)-3-hydroxycyclobutyl)carbamate (5.0 g, 26.70 mmol, 1.0 equiv) in THF (50 mL) was added in small portions sodium hydride (60% dispersion in mineral oil, 1.60 g, 40.10 mmol, 1.5 equiv) at 0° C. and stirred for 30 min. Benzyl bromide (5.48 g, 32.04 mmol, 1.2 equiv) was added and the reaction mixture was stirred at room temperature for 1 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (5% ethyl acetate in hexane) to afford 21.1 (2.3 g, 31%). MS(ES): m/z 278.4 [M+H]⁺.

Synthesis of compound 21.2. To a solution of 21.1 (2.3 g, 0.21 mmol, 1.0 equiv) in DCM (5.0 mL) at 0° C. was added dropwise trifluoroacetic acid (1.5 mL) and stirred for 50 min at room temperature. It was concentrated under reduced pressure to afford 21.2 (1.1 g). The residue was used in the next step without purification. MS(ES): m/z 178.3 [M+H]⁺.

Synthesis of compound 21.3. Compound 21.3 was prepared from compound 1.8 and 21.2 following the procedure described in the synthesis of compound 1.9. The product was purified by flash column chromatography on silica gel (Combiflash®, 60% ethyl acetate in hexane). MS(ES): m/z 574.2 and 576.2 [M+H]⁺.

Synthesis of compound 21.4. Compound 21.4 was prepared from compound 21.3 following the procedure described in the synthesis of compound 1.10. The product was triturated with pentane, dried under reduced pressure and used in the next step without purification. MS(ES): m/z 546.3 and 548.3 [M+H]⁺.

Synthesis of compound 21.5. Compound 21.5 was prepared from compound 21.4 following the procedure described in the synthesis of compound 1.11. The product was purified by flash column chromatography on silica gel (Combiflash®, 1.8% methanol in DCM). MS(ES): m/z 543.4 and 545.4 [M+H]⁺.

Synthesis of compound 21.6. Compound 21.6 was prepared from compound 21.5 following the procedure described in the synthesis of compound 1.12. The product was purified by flash column chromatography on silica gel (1.5% methanol in DCM). MS(ES): m/z 557.3 and 559.3 [M+H]⁺.

Synthesis of compound 21.7. Compound 21.7 was prepared from compound 21.6 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (Combiflash®, 2.9% methanol in DCM). MS(ES): m/z 559.5 [M+H]⁺.

Synthesis of compound 21.8. To a solution of 21.7 (0.265 g, 0.47 mmol, 1.0 equiv) in DCM (5.0 mL) at 0° C. was added dropwise triflic acid (1.5 mL) and stirred for 15 min at room temperature. It was quenched with saturated sodium bicarbonate solution and extracted with ethyl acetate. The combined organic layers were washed with brine, dried over anhydrous sodium sulfate, filtered and concentrated under reduced pressure to afford compound 21.8 which was used in the next step without purification (0.115 g, 52%). MS(ES): m/z 469.4 [M+H]⁺.

Synthesis of I-21. A solution of 21.8 (0.060 g, 0.128 mmol, 1.0 equiv), 4-dimethylaminopyridine (0.023 g, 0.192 mmol, 1.5 equiv) and N,N-diisopropylethylamine (0.049 g, 0.384 mmol, 3.0 equiv) in DCM (4.0 mL) at 0° C. was added 4-nitrophenyl chloroformate (0.038 g, 0.192 mmol, 1.5 equiv) dropwise and stirred for 20 min. It was concentrated under reduced pressure. The residue was dissolved in DMF (2 mL) was added N,N-diisopropylethylamine (0.099 g, 0.768 mmol, 6.0 equiv) and oxetan-3-amine (0.028 g, 0.384 mmol, 1.5 equiv). The reaction mixture was stirred at 60° C. for 2 h. It was poured into water and extracted with ethyl acetate. The combined organic layers were washed with brine, dried over anhydrous sodium sulfate, filtered and concentrated under reduced pressure. The residue was purified by flash column chromatography on silica gel (Combiflash®, 2.2% methanol in DCM) to afford I-21 (0.026 g, 36%). MS(ES): m/z 568.47 [M+H]⁺, ¹H NMR (DMSO-d₆, 400 MHz): δ 8.40 (s, 1H), 8.16 (s, 1H), 8.10 (s, 1H), 7.93 (d, J=7.2 Hz, 1H), 7.84 (d, J=8.4 Hz, 1H), 7.78 (s, 1H), 7.41 (d, J=7.2 Hz, 1H), 7.29 (s, 1H), 7.11 (s, 1H), 5.76 (s, 1H), 4.56-4.54 (m, 3H), 4.38-4.35 (m, 2H), 4.14 (s, 3H), 3.67 (s, 3H), 3.36-3.33 (m, 2H), 3.13-3.07 (m, 2H), 2.85-2.76 (m, 2H), 1.86 (bs, 1H), 1.50 (bs, 1H).

### Example I-22: (1s,3S)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclobutyl (((R)-THF-2-yl)methyl)carbamate

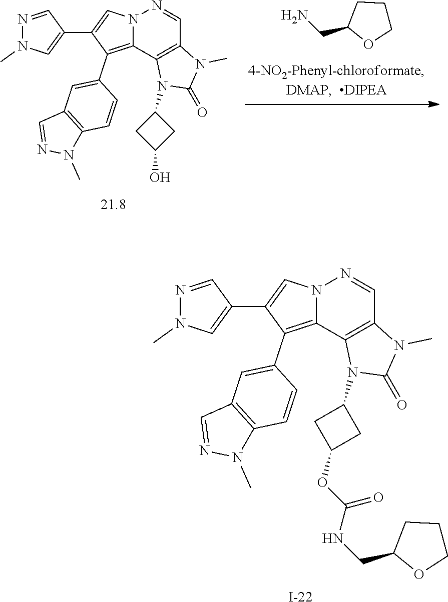

Synthesis of I-22. A solution of 21.8 (0.055 g, 0.117 mmol, 1.0 equiv), 4-dimethylaminopyridine (0.021 g, 0.176 mmol, 1.5 equiv) and N,N-diisopropylethylamine (0.045 g, 0.351 mmol, 3.0 equiv) in DCM (4.0 mL) at 0° C. was added 4-nitrophenyl chloroformate (0.035 g, 0.176 mmol, 1.5 equiv) dropwise and stirred for 20 min. It was concentrated under reduced pressure. The residue was dissolved in DMF (2 mL) and added N,N-diisopropylethylamine (0.099 g, 0.768 mmol, 6.0 equiv) and (R)-(THF-2-yl)methanamine (0.035 g, 0.352 mmol, 1.5 equiv). The reaction mixture was stirred at 60° C. for 2 h. It was poured into water and extracted with ethyl acetate. The combined organic layers were washed with brine, dried over anhydrous sodium sulfate, filtered and concentrated under reduced pressure. The residue was purified by flash column chromatography on silica gel (Combiflash®, 2.4% methanol in DCM) to afford I-22 (0.025 g, 36%). MS(ES): m/z 596.5 [M+H]⁺, ¹H NMR (DMSO-d₆, 400 MHz): 1H-NMR (400 MHz, DMSO-d6): δ 8.40 (s, 1H), 8.16 (s, 1H), 8.11 (s, 1H), 7.85 (d, J=8.4 Hz, 1H), 7.78 (s, 1H), 7.40 (dd, J=7.2 Hz, J=1.2 Hz, 1H), 7.29 (s, 1H), 7.14 (t, J=6.0 Hz, 1H), 7.11 (s, 1H), 4.15 (s, 3H), 3.71-3.35 (m, 2H), 3.34 (s, 3H), 3.33-3.31 (m, 1H), 3.31 (s, 3H), 3.12-3.07 (m, 2H), 2.94-2.78 (m, 4H), 1.85-1.78 (m, 3H), 1.49-1.45 (m, 2H).

### Example I-23: methyl ((1R,3R)-3-(3-methyl-9-(1-methyl-1H-indazol-5-yl)-8-(2-methyl-2H-1,2,3-triazol-4-yl)-2-oxo-2,3-dihydro-1H-imidazo[4,5-d]pyrrolo[1,2-b]pyridazin-1-yl)cyclopentyl)carbamate

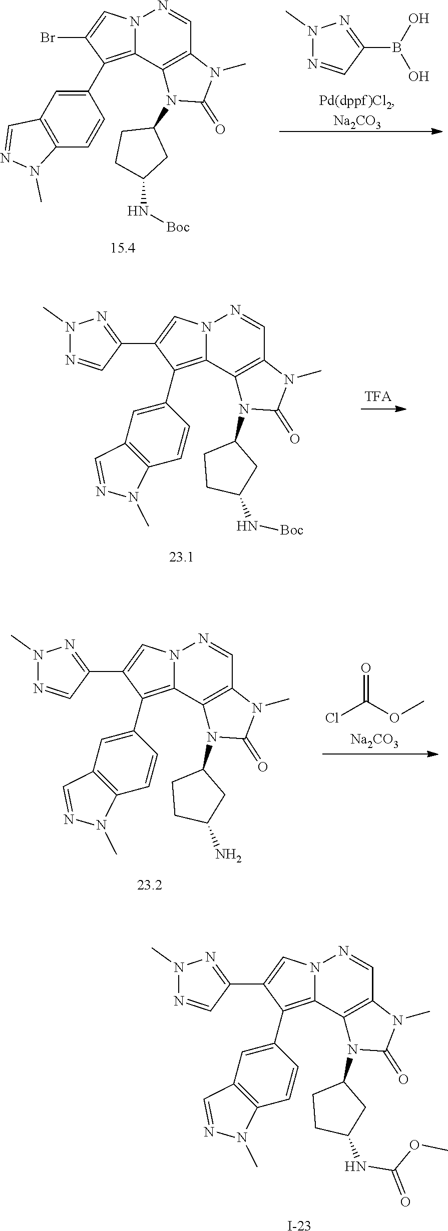

Synthesis of compound 23.1. Compound 23.1 was prepared from compound 15.4 and (2-methyl-2H-1,2,3-triazol-4-yl)boronic acid following the procedure described in the synthesis of compound 1.13. The product was purified by flash column chromatography on silica gel (Combiflash®, 0-3.2% methanol in DCM). MS(ES): m/z 583.2 [M+H]⁺.

Synthesis of compound 23.2. Compound 23.2 was prepared from compound 23.1 following the procedure described in the synthesis of compound 1.14. The product was triturated with pentane and dried under reduced pressure. It was used in the next step without purification. MS(ES): m/z 483.1 [M+H]⁺.

Synthesis of I-23. Compound I-23 was prepared from compound 23.2 following the procedure described in the synthesis of compound I-1. The product was purified by flash column chromatography on silica gel (Combiflash®, 0-2.6% methanol in DCM). MS(ES): m/z 541.5 [M+H]⁺, ¹H NMR (DMSO-d₆, 400 MHz): δ 8.47 (s, 1H), 8.19 (d, J=3.2 Hz, 1H), 8.07-8.05 (m, 1H), 7.78-7.76 (m, 2H), 7.44-7.40 (m, 1H), 6.60 (s, 1H), 6.46 (s, 1H), 4.23 (s, 3H), 4.06 (s, 3H), 3.88-3.82 (m, 1H), 3.48 (s, 3H), 3.32 (s, 3H), 3.09-3.07 (m, 1H), 2.01-1.98 (m, 1H), 1.84-1.72 (m, 1H), 1.52-1.51 (m, 1H), 1.19-1.09 (m, 2H), 0.36 (s, 0.3H), −0.13 (s, 0.7H).

### Example I-24: methyl ((1S,3S)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

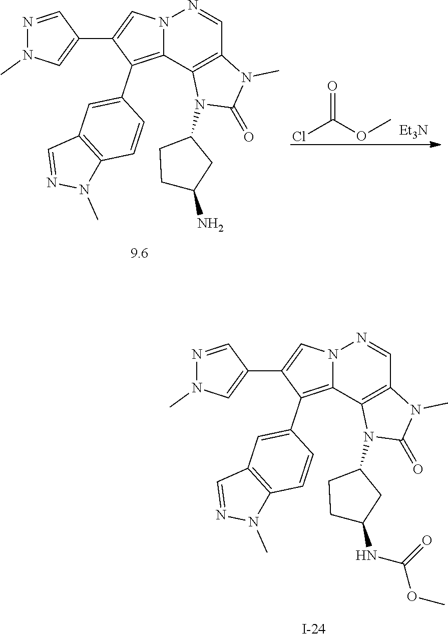

Synthesis of I-24. Compound I-24 was prepared from compound 9.6 following the procedure described in the synthesis of compound I-1. The product was purified by flash column chromatography on silica gel (Combiflash®, 3.3% methanol in DCM). MS(ES): m/z 540.42 [M+H]⁺, ¹H-NMR (400 MHz, DMSO-d₆): δ 8.38 (s, 1H), 8.14 (d, J=4.0 Hz, 1H), 8.06 (d, J=7.2 Hz, 1H), 7.76 (d, J=8.8 Hz, 2H), 7.41-7.37 (m, 1H), 7.26-7.24 (m, 1H), 7.03-6.92 (m, 1H), 6.68-6.66 (m, 1H), 4.17-3.09 (m, 3H), 3.83 (bs, 1H), 3.66 (s, 3H), 3.46 (s, 2H), 3.45 (bs, 1H), 3.39 (s, 3H), 2.00 (bs, 1H), 1.71 (bs, 1H), 1.51 (bs, 1H), 1.13-1.06 (m, 2H), 1.07 (bs, 1H), 0.80 (bs, 1H).

### Example I-25: methyl ((1R,3R)-3-(3-methyl-9-(1-methyl-1H-indazol-5-yl)-2-oxo-8-(tetrahydro-2H-pyran-4-yl)-2,3-dihydro-1H-imidazo[4,5-d]pyrrolo[1,2-b]pyridazin-1-yl)cyclopentyl)carbamate

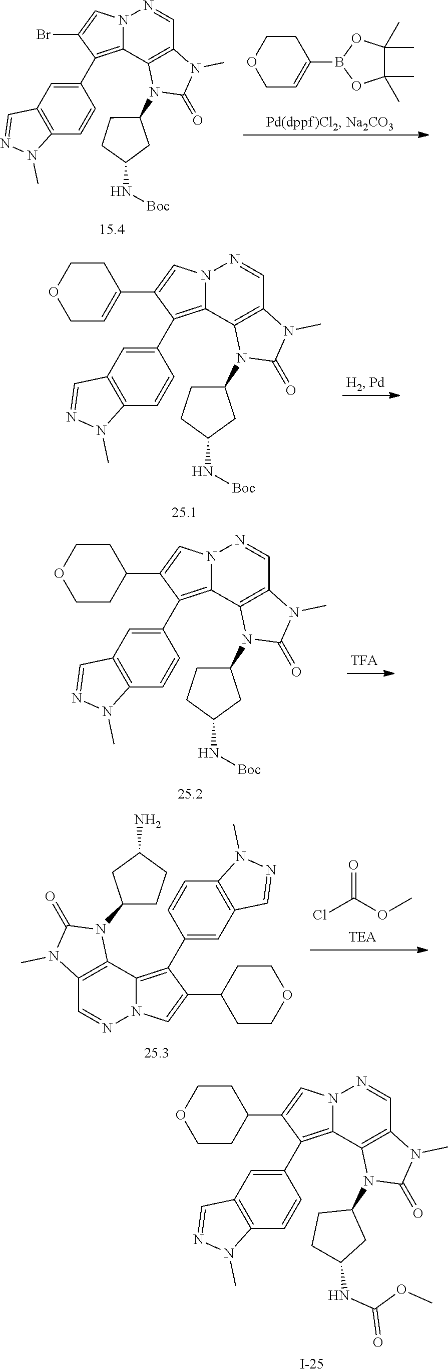

Synthesis of compound 25.1. Compound 25.1 was prepared from compound 15.4 and 2-(3,6-dihydro-2H-pyran-4-yl)-4,4,5,5-tetramethyl-1,3,2-dioxaborolane following the procedure described in the synthesis of compound 1.13. The product was purified by flash column chromatography on silica gel (2.1% methanol in DCM). MS(ES): m/z 584.70 [M+H]⁺.

Synthesis of compound 25.2. A mixture of 25.1 (0.200 g, 0.342 mmol, 1.0 equiv) and 10% palladium on carbon (0.100 g) in methanol (15 mL) was stirred under hydrogen atmosphere at room temperature for 12 h. It was filtered through a pad of Celite®. The filtrate was concentrated under reduced pressure. The residue was washed with diethyl ether to afford 25.2 (0.118 g, 59%). MS(ES): m/z: 586.7 [M+H]⁺.

Synthesis of compound 25.3. Compound 25.3 was prepared from compound 25.2 following the procedure described in the synthesis of compound 1.14. The product was triturated with pentane and dried under reduced pressure. It was used in the next step without purification. MS(ES): m/z 486.6 [M+H]⁺.

Synthesis of I-25. Compound I-25 was prepared from compound 25.3 following the procedure described in the synthesis of compound I-1. The product was purified by flash column chromatography on silica gel (Combiflash®, 3.4% methanol in DCM). MS(ES): m/z 544.9 [M+H]⁺, ¹H NMR (DMSO-d₆, 400 MHz): δ 8.36 (s, 1H), 8.07-8.043 (d, J=2.8 Hz, 1H), 7.81 (s, 1H), 7.48 (m, 2H), 7.38-7.36 (m, 1H), 6.66 (s, 0.5H), 6.29 (s, 0.5H), 4.13 (s, 3H), 3.85-3.79 (m, 4H), 3.48 (m, 2H), 3.15-3.310 (m, 2H), 2.009 (m, 2H), 1.70 (s, 6H), 1.54-1.51 (m, 2H), 1.35 (m, 2H), 1.14- (m, 1H), 0.85-0.73 (m, 1H), 0.32 (s, 0.5H), −0.19 (s, 0.5H).

### Example I-26: methyl ((1R,3R)-3-(8-(3,6-dihydro-2H-pyran-4-yl)-3-methyl-9-(1-methyl-1H-indazol-5-yl)-2-oxo-2,3-dihydro-1H-imidazo[4,5-d]pyrrolo[1,2-b]pyridazin-1-yl)cyclopentyl)carbamate

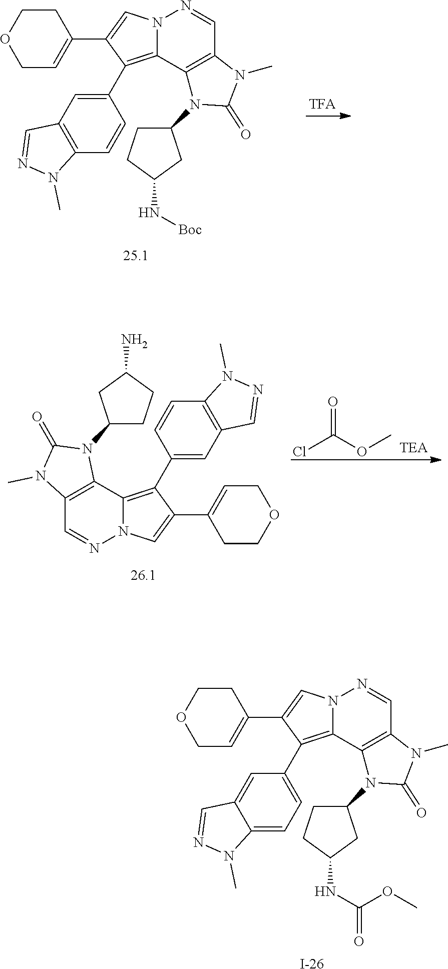

Synthesis of compound 26.1: Compound 26.1 was prepared from compound 25.1 following the procedure described in the synthesis of compound 1.14. The product was triturated with pentane and dried under reduced pressure. It was used in the next step without purification. MS(ES): m/z 484.62 [M+H]⁺.

Synthesis of I-26. Compound I-26 was prepared from compound 26.1 following the procedure described in the synthesis of compound I-1. The product was purified by silica gel flash column chromatography on silica gel (3.3% methanol in DCM). MS(ES): m/z 542.17 [M+H]⁺, 1H-NMR (400 MHz, DMSO-d₆): δ 8.39 (s, 1H), 8.06-8.04 (m, 1H), 7.95 (s, 1H), 7.73 (s, 2H), 7.40 (d, J=7.6 Hz, 1H), 6.68 (s, 0.6H), 6.28 (s, 0.4H), 5.42-5.36 (m, 1H), 4.12 (s, 3H), 3.88 (s, 3H), 3.63-3.62 (m, 2H), 3.39 (s, 3H), 3.32 (s, 3H), 2.29-2.03 (m, 2H), 1.95 (bs, 1H), 1.72-1.71 (m, 1H), 1.47 (bs, 1H), 1.33 (bs, 1H), 1.10-0.01 (m, 1H), 0.99-0.96 (m, 1H).

### Example I-27: methyl ((1R,3R)-3-(9-(1,1-dimethyl-1,3-dihydroisobenzofuran-5-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

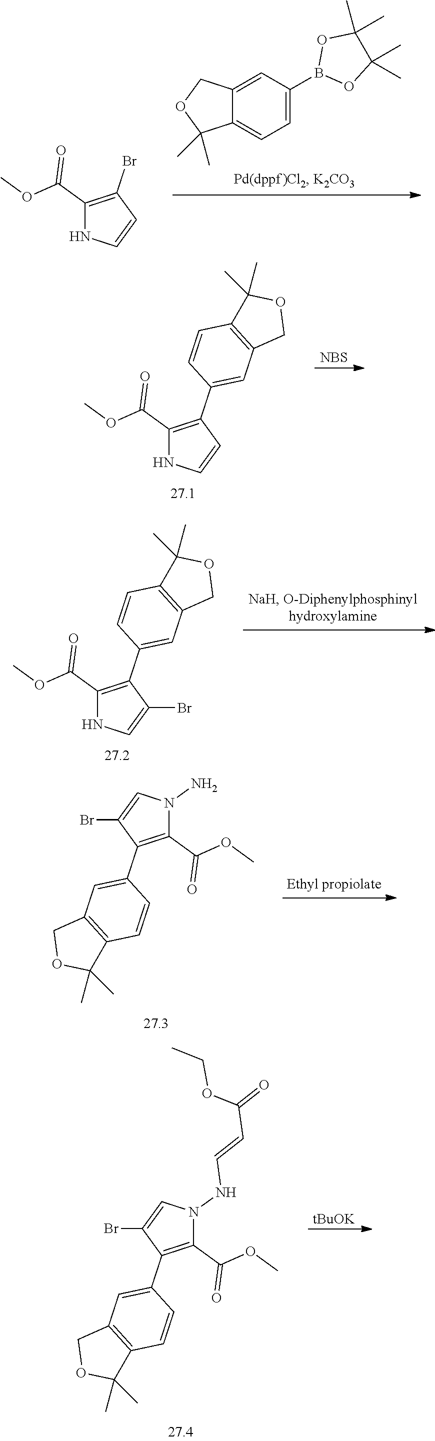

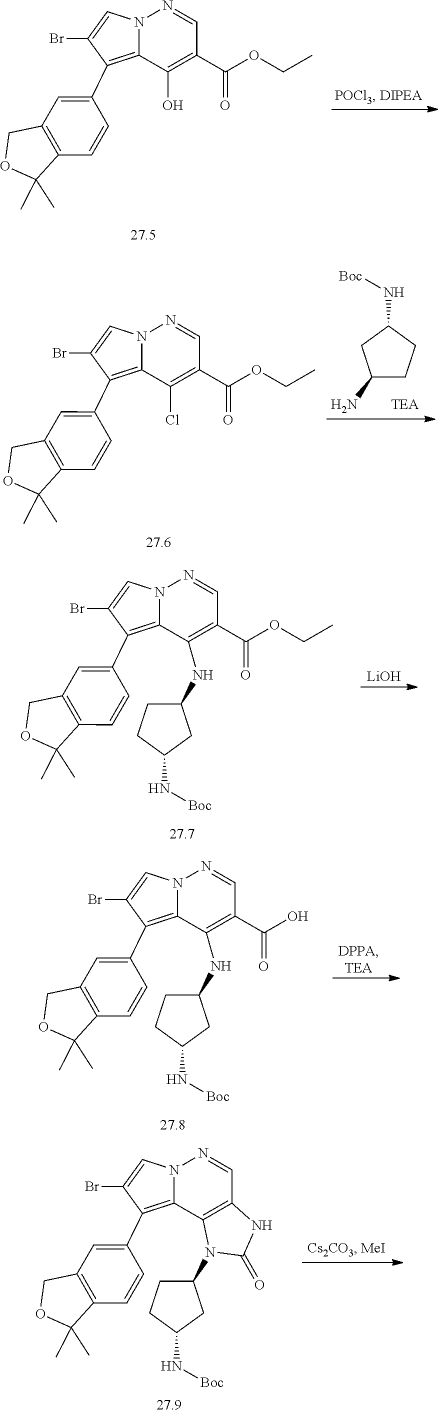

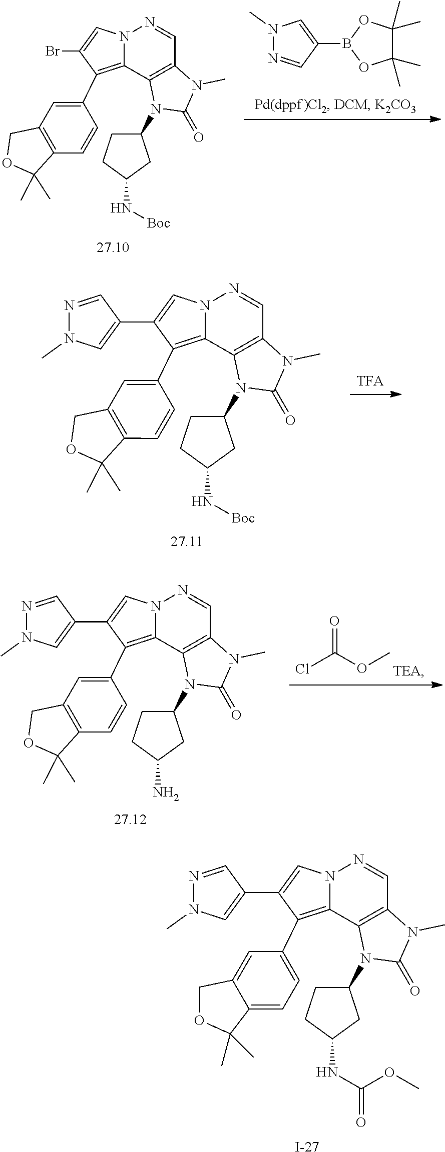

Synthesis of compound 27.1. A mixture of methyl 3-bromo-1H-pyrrole-2-carboxylate (10 g, 49.10 mmol, 1 equiv), 2-(1,1-dimethyl-1,3-dihydroisobenzofuran-5-yl)-4,4,5,5-tetramethyl-1,3,2-dioxaborolane (17.46 g, 63.72 mmol, 1.3 equiv) and potassium carbonate (20.29 g, 147.0 mmol, 3 equiv) in 1,4-dioxane:water (8:2, 500 mL) was degassed by bubbling through a stream of argon for 20 min. 1,1′-Bis(diphenylphosphino)ferrocene]dichloropalladium(II) (3.58 g, 4.91 mmol, 0.1 equiv) was added and stirred at 100° C. for 4 h. It was cooled to room temperature, transferred into ice water and extracted with ethyl acetate. The combined organic layers were washed with brine, dried over anhydrous sodium sulfate, filtered and concentrated under reduced pressure. The residue was purified by flash column chromatography on silica gel (Combiflash®, 28% ethyl acetate in hexane) to afford 27.1 (6.1 g, 46%). MS (ES): m/z 272.5 [M+H]⁺.

Synthesis of compound 27.2. To a solution of 27.1 (6.1 g, 22.50 mmol, 1 equiv) in THF:water (3:1, 400 mL) was added N-bromosuccinimide (4.0 g, 22.50.0 mmol, 1 equiv) at 0° C. and stirred for 15 min. It was quenched with water and extracted with ethyl acetate. The combined organic layers were washed with brine, dried over anhydrous sodium sulfate, filtered and concentrated under reduced pressure. The residue was purified by flash column chromatography on silica gel (Combiflash®, 26% ethyl acetate in hexane) to afford 27.2 (5.1 g, 65%). MS (ES): m/z 348.1 and 351.1 [M+H]⁺.

Synthesis of compound 27.3. To a solution of 27.2 (5.1, 9.28 mmol, 1.0 equiv) in DMF (1 L) was added sodium hydride (0.557 g, 13.39 mmol, 1.5 equiv) at 0° C. and stirred for 30 min. O-diphenylphosphinyl hydroxylamine (3.1 g, 13.39 mmol, 1.5 equiv) was added at 0° C. and the mixture was stirred at room temperature. It was diluted with water and extracted with ethyl acetate. The combined organic layers were washed with brine, dried over anhydrous sodium sulfate, filtered and concentrated under reduced pressure. The residue was purified by flash column chromatography on silica gel (Combiflash®, 28% ethyl acetate in hexane) to afford 27.3 (4.9 g, 92%). MS(ES): m/z 366.3 and 368.3 [M+H]⁺.

Synthesis of compound 27.4. To a stirred solution of 27.3 (4.9 g, 13.42 mmol, 1.0 equiv) in methanol (300 mL) was added ethyl propiolate (15.46 g, 161.0 mmol, 12 equiv) at room temperature. The reaction mixture was stirred at 70° C. for 24 h. It was concentrated under reduce pressure. The residue was purified by flash column chromatography on silica gel (Combiflash®, 32% ethyl acetate in hexane) to afford 27.4 (3.3 g, 53%). MS(ES): m/z 464.3 and 466.3 [M+H]⁺.

Synthesis of compound 27.5. To a stirred solution of 27.4 (3.3 g, 9.04 mmol, 1.0 equiv) in ethanol (200 mL) was added potassium tert-butoxide (3.03 g, 27.12 mmol, 3 equiv) at room temperature. The reaction mixture was stirred at 70° C. for 2 h. It was cooled to room temperature and transferred into water. The precipitation was collected by filtration. It was dissolved in DCM, dried over anhydrous sodium sulfate, filtered and concentrated under reduced pressure. The residue was purified by flash column chromatography on silica gel (Combiflash®, 35% ethyl acetate in hexane) to afford 27.5 (2.8 g, 91%). MS(ES): m/z 432.2 and 434.2 [M+H]⁺.

Synthesis of compound 27.6. A solution of 27.5 (2.8 g, 6.48 mmol, 1.0 equiv), phosphorous oxychloride (28 mL), and N,N-diisopropylethylamine (9 mL) was stirred at 80° C. for 3 h. It was cooled to room temperature and transferred into water. The mixture was basified with a saturated solution of sodium bicarbonate and extracted with ethyl acetate. The combined organic layers were washed with brine, dried over anhydrous sodium sulfate, filtered and concentrated under reduced pressure. The residue was purified by flash column chromatography on silica gel (Combiflash®, 28% ethyl acetate in hexane) to afford 27.6 (2.2 g, 75%). MS(ES): m/z 450.2 and 452.2 [M+H]⁺.

Synthesis of compound 27.7. Compound 27.7 was prepared from compound 27.6 and tert-butyl ((1R,3R)-3-aminocyclopentyl)carbamate following the procedure described in the synthesis of compound 1.9. The product was purified by flash column chromatography on silica gel (Combiflash®, 36% ethyl acetate in hexane). MS(ES): m/z 614.5 and 616.5 [M+H]⁺.

Synthesis of compound 27.8. Compound 27.8 was prepared from compound 27.7 following the procedure described in the synthesis of compound 1.10. The product was used without purification. MS(ES): m/z 586.5 and 588.5 [M+H]⁺.

Synthesis of compound 27.9. Compound 27.9 was prepared from compound 27.8 following the procedure described in the synthesis of compound 1.11. The product was purified by flash column chromatography on silica gel (Combiflash®, 52% ethyl acetate in hexane). MS(ES): m/z 583.5 and 585.5 [M+H]⁺.

Synthesis of compound 27.10. Compound 27.10 was prepared from compound 27.9 following the procedure described in the synthesis of compound 1.12. The product was purified by flash column chromatography on silica gel (Combiflash®, 48% ethyl acetate in hexane). MS(ES): m/z 597.18 and 599.18 [M+H]⁺.

Synthesis of compound 27.11. Compound 27.11 was prepared from compound 27.10 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (Combiflash®, 1.9% methanol in DCM). MS(ES): m/z 598.26 [M+H]⁺.

Synthesis of compound 27.12. Compound 27.12 was prepared from compound 27.11 following the procedure described in the synthesis of compound 1.14. The product was used in the next step without purification. MS(ES): m/z 498.26 [M+H]⁺.

Synthesis of compound I-27. Compound I-27 was prepared from compound 27.12 following the procedure described in the synthesis of compound I-1. The product was purified by flash column chromatography on silica gel (Combiflash®, 2.6% methanol in DCM). MS(ES): m/z 556.26 [M+H]⁺, ¹H NMR (DMSO-d₆, 400 MHz): δ 8.39 (s, 1H), 8.13 (s, 1H), δ 7.4-7.25 (m, 4H), 7.01-6.85 (m, 2H), 5.17-4.91 (m, 2H), 4.05-4.04 (m, 1H), 3.93-3.88 (m, 1H), 3.70 (s, 3H), 3.41 (s, 3H), 3.36 (s, 3H), 2.04 (bs, 1H), 1.85 (m, 2H), 1.53-1.50 (m, 6H), 1.34 (bs, 1H), 1.24-1.16 (m, 2H).

### Example I-28: methyl ((1R,3R)-3-(9-(1,1-dimethyl-1,3-dihydroisobenzofuran-5-yl)-3-methyl-8-(2-methyl-2H-1,2,3-triazol-4-yl)-2-oxo-2,3-dihydro-1H-imidazo[4,5-d]pyrrolo[1,2-b]pyridazin-1-yl)cyclopentyl)carbamate

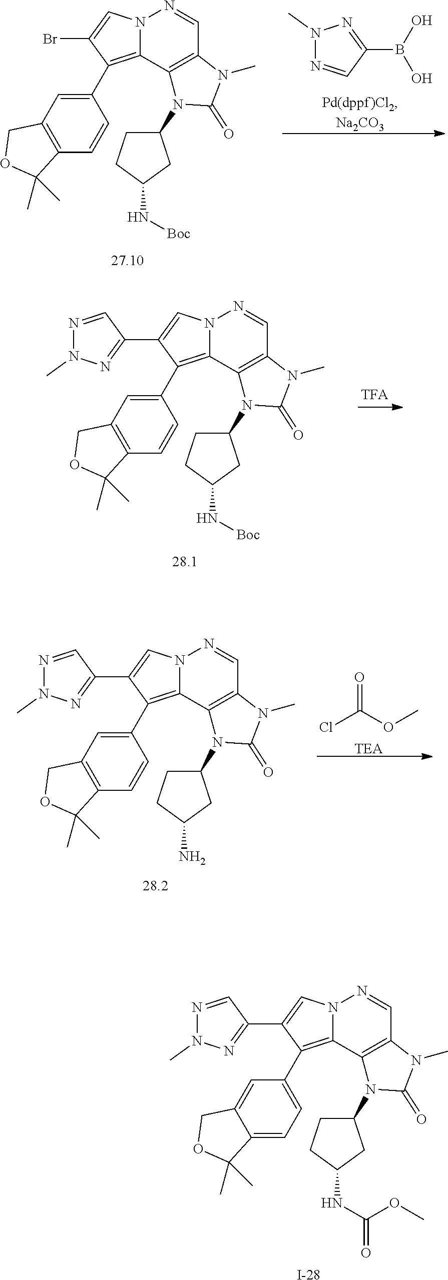

Synthesis of compound 28.1. Compound 28.1 was prepared from compound 27.10 and 1 (2-methyl-2H-1,2,3-triazol-4-yl)boronic acid following the procedure described in the synthesis of compound 1.13. The product was purified by flash column chromatography on silica gel (2.5% methanol in DCM). MS(ES): m/z 599.3 [M+H]⁺.

Synthesis of compound 28.2. Compound 28.2 was prepared from compound 28.1 following the procedure described in the synthesis of compound 1.14. The product was used in the next step without purification. MS(ES): m/z 499.5 [M+H]⁺.

Synthesis of compound I-28. Compound I-28 was prepared from compound 28.2 following the procedure described in the synthesis of compound I-1. The product was purified by flash column chromatography on silica gel (Combiflash®, 3.5% methanol in DCM). MS(ES): m/z 557.5 [M+H]⁺, ¹H NMR (DMSO-d₆, 400 MHz): δ 8.47 (s, 1H), 8.17 (S, 1H), 7.38-7.34 (m, 2H), 7.27 (s, 1H), 6.64 (s, 1H), 6.56 (s, 1H), 4.08 (s, 3H), 3.90-3.87 (d, J=12 Hz, 1H), 3.48 (S, 3H), 3.32 (s, 3H), 2.02-1.98 (d, J=16 Hz 1H), 1.52-1.49 (m, 6H), 1.36-1.29 (m, 2H), 1.23 (s, 6H).

### Example I-29: methyl ((1R,3R)-3-(9-(1,1-dimethyl-1,3-dihydroisobenzofuran-5-yl)-3-methyl-8-(1-methyl-1H-1,2,3-triazol-4-yl)-2-oxo-2,3-dihydro-1H-imidazo[4,5-d]pyrrolo[1,2-b]pyridazin-1-yl)cyclopentyl)carbamate

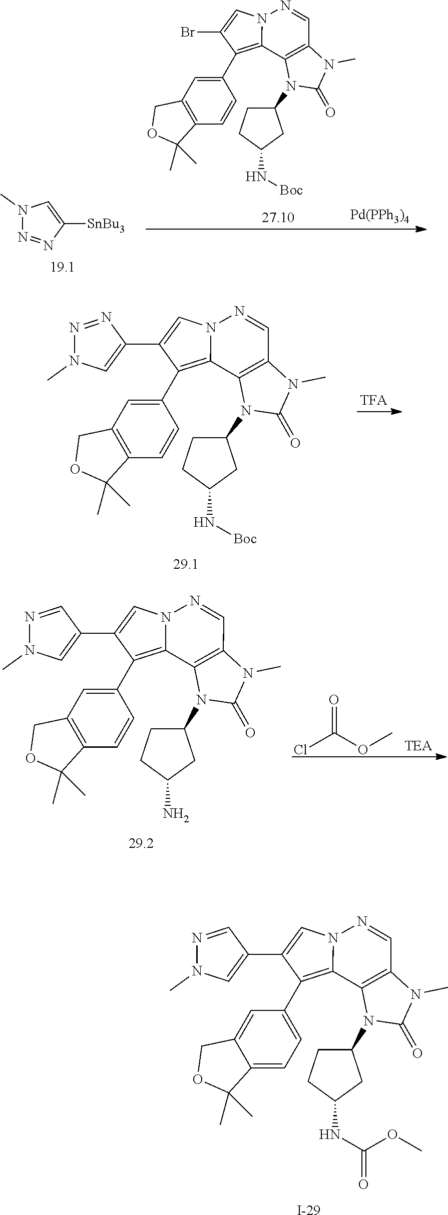

Synthesis of compound 29.1. Compound 29.1 was prepared from compound 27.10 and 19.1 following the procedure described in the synthesis of compound 19.2. The product was purified by flash column chromatography on silica gel (Combiflash®, 2.1% methanol in DCM). MS (ES): m/z 599.5 [M+H]⁺.

Synthesis of compound 29.2. Compound 29.2 was prepared from compound 29.1 following the procedure described in the synthesis of compound 1.14. The product was used in the next step without purification. MS(ES): m/z 499.26 [M+H]⁺.

Synthesis of compound I-29. Compound I-29 was prepared from compound 29.2 following the procedure described in the synthesis of compound I-1. The product was purified by flash column chromatography on silica gel (Combiflash®, 2.6% methanol in DCM). MS(ES): m/z 557.26 [M+H]⁺, ¹H NMR (DMSO-d₆, 400 MHz): δ 8.46 (s, 1H), 8.22 (s, 1H), δ 7.41-7.32 (m, 2H), 7.24 (s, 1H), 7.03-6.93 (m, 1H), 5.16-4.91 (m, 2H), 4.17 (bs, 1H), 3.90 (s, 3H), 3.85 (s, 1H), 3.40 (s, 3H), 3.39 (s, 3H), 2.03-1.99 (m, 1H), 1.90-1.85 (m, 3H), 1.53-1.49 (m, 6H), 1.33 (bs, 1H), 1.22 (s, 1H) 1.09-1.07 (m, 1H).

### Example I-30: methyl ((1r,3r)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclobutyl)carbamate

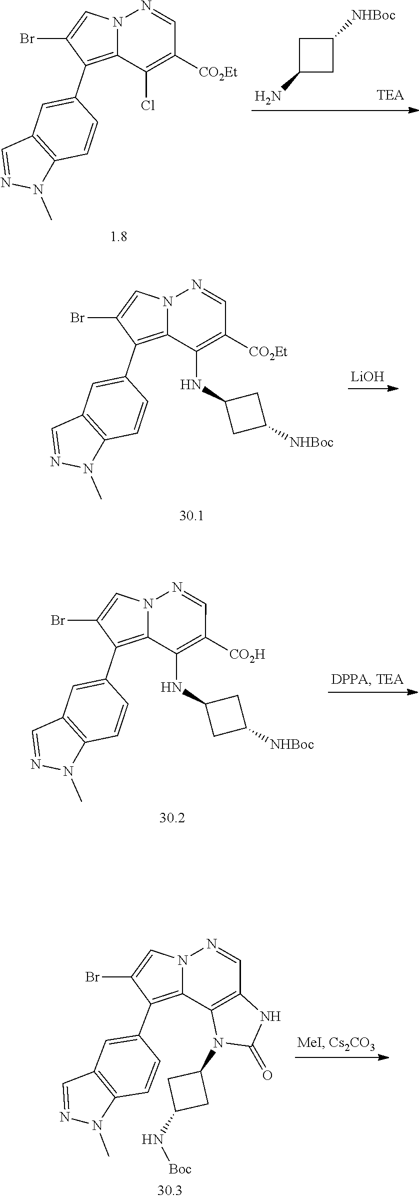 [SMILES: CCOC(=O)c1cnn2cc(Br)c(-c3ccc4c(cnn4C)c3)c2c1Cl ; CC(C)(C)OC(=O)N[C@H]1C[C@H](N)C1 ; CCOC(=O)c1cnn2cc(Br)c(-c3ccc4c(cnn4C)c3)c2c1N[C@H]1C[C@H](NC(=O)OC(C)(C)C)C1 ; "LiOH" ; Cn1ncc2cc(-c3c(Br)cn4ncc(C(=O)O)c(N[C@H]5C[C@H](NC(=O)OC(C)(C)C)C5)c34)ccc21 ; Cn1ncc2cc(-c3c(Br)cn4ncc5[nH]c(=O)n([C@H]6C[C@H](NC(=O)OC(C)(C)C)C6)c5c34)ccc21]

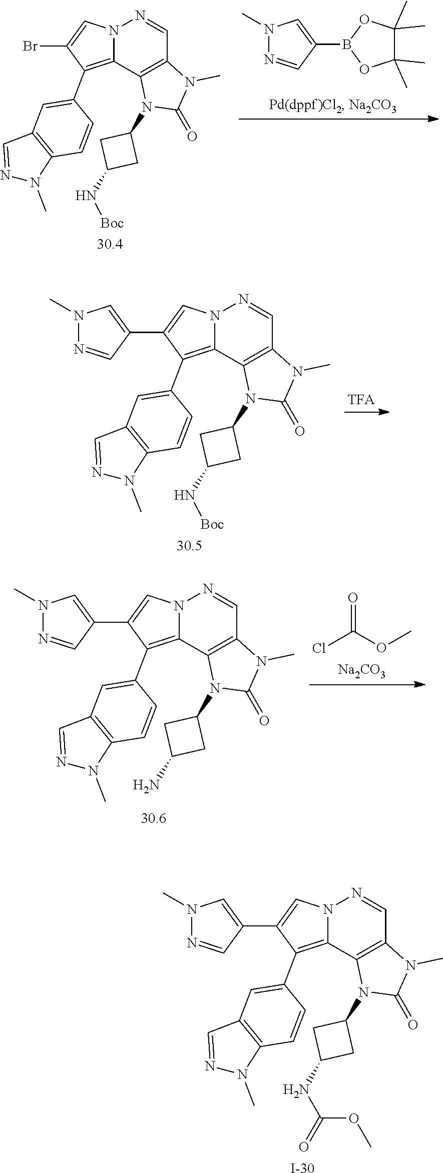

Synthesis of compound 30.1. Compound 30.1 was prepared from compound 1.8 and tert-butyl ((1r,3r)-3-aminocyclobutyl)carbamate following the procedure described in the synthesis of compound 1.9. The product was purified by flash column chromatography on silica gel (Combiflash®, 50% ethyl acetate in hexane). MS(ES): m/z 583.2 and 585.2 [M+H]⁺.

Synthesis of compound 30.2. Compound 30.2 was prepared from compound 30.1 following the procedure described in the synthesis of compound 1.10. The product was triturated with pentane, dried under vacuum and used in the next step without purification. MS(ES): m/z 555.4 and 557.4 [M+H]⁺.

Synthesis of compound 30.3. Compound 30.3 was prepared from compound 30.2 following the procedure described in the synthesis of compound 1.11. The product was purified by flash column chromatography on silica gel (Combiflash®, 2.1% methanol in DCM). MS(ES): m/z 552.4 and 554.4 [M+H]⁺.

Synthesis of compound 30.4. Compound 30.4 was prepared from compound 30.3 following the procedure described in the synthesis of compound 1.12. The product was purified by flash column chromatography on silica gel (1.7% methanol in DCM). MS(ES): m/z 566.3 and 568.3 [M+H]⁺.

Synthesis of compound 30.5. Compound 30.5 was prepared from compound 30.4 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (Combiflash®, 3.1% methanol in DCM). MS(ES): m/z 568.5 [M+H]⁺.

Synthesis of compound 30.6. Compound 30.6 was prepared from compound 30.5 following the procedure described in the synthesis of compound 1.14. The product was used in the next step without purification. MS(ES): m/z 468.4 [M+H]⁺.

Synthesis of I-30. Compound I-30 was prepared from compound 30.6 following the procedure described in the synthesis of compound I-1. The product was purified by flash column chromatography on silica gel (Combiflash®, 2.8% methanol in DCM). MS(ES): m/z 526.48 [M+H]⁺, ¹H NMR (DMSO-d₆, 400 MHz): δ 8.40 (s, 1H), 8.12 (s, 1H), 8.02 (s, 1H), 7.77 (d, J=8.0 Hz, 1H), 7.66 (s, 1H), 7.36 (d, J=7.6 Hz, 1H), 7.27 (s, 1H), 6.94 (s, 1H), 6.29 (s, 1H), 4.13 (s, 3H), 3.92 (s, 1H), 3.76-3.75 (m, 1H), 3.66 (s, 3H), 3.46 (s, 3H), 3.37 (s, 3H), 2.83 (s, 1H), 2.67 (s, 1H), 1.67 (s, 1H), 1.23-1.20 (m, 1H).

### Example I-31: methyl ((1R,3R)-3-(8-(3-methoxy-1-methyl-1H-pyrazol-4-yl)-3-methyl-9-(1-methyl-1H-indazol-5-yl)-2-oxo-2,3-dihydro-1H-imidazo[4,5-d]pyrrolo[1,2-b]pyridazin-1-yl)cyclopentyl)carbamate

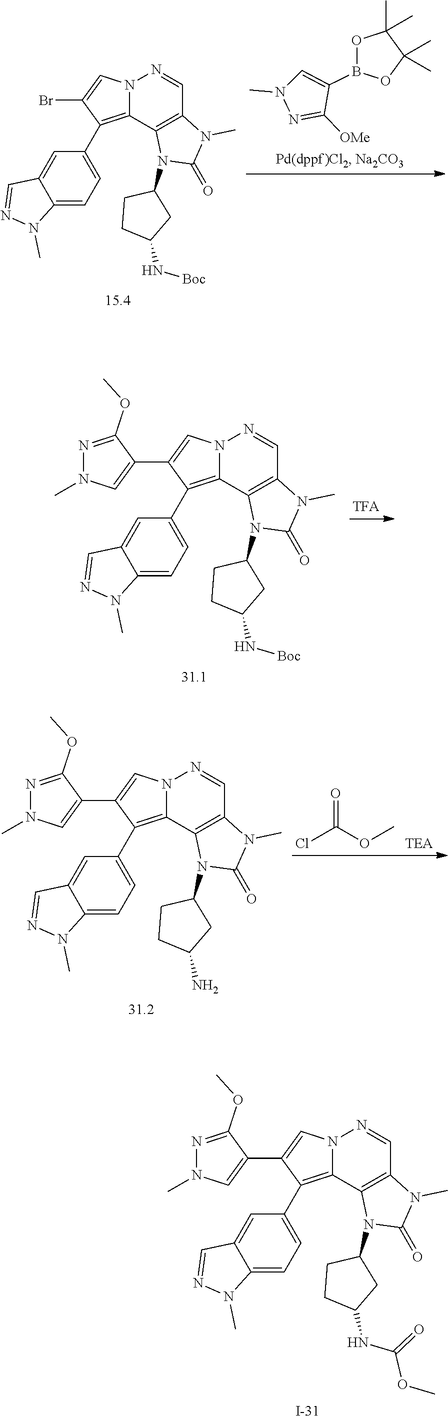

Synthesis of compound 31.1. Compound 31.1 was prepared from compound 15.4 and 3-methoxy-1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (2.5% methanol in DCM). MS(ES): m/z 612.6 [M+H]⁺.

Synthesis of compound 31.2. Compound 31.2 was prepared from compound 31.1 following the procedure described in the synthesis of compound 1.14. The product was used in the next step without purification. MS(ES): m/z 512.4 [M+H]⁺.

Synthesis of I-31. Compound I-31 was prepared from compound 31.2 following the procedure described in the synthesis of compound I-31. The product was purified by silica gel flash column chromatography on silica gel (3.6% methanol in DCM). MS(ES): m/z 570.22 [M+H]⁺, ¹H-NMR (400 MHz, DMSO-d₆): δ 8.37 (s, 1H), 8.08-8.01 (m, 2H), 7.78-7.74 (m, 2H), 7.40-7.32 (m, 1H), 6.70 (s, 1H), 6.31-6.21 (m, 1H), 4.15 (s, 3H), 3.88 (s, 3H), 3.48-3.40 (m, 6H), 1.97 (bs, 1H), 1.73 (bs, 1H), 1.48 (bs, 1H), 1.33-0.92 (s, 6H), 0.75 (s, 1H), 0.30 (s, 1H).

### Example I-32: methyl ((1R,3R)-3-(9-(1,1-dimethyl-1,3-dihydroisobenzofuran-5-yl)-3-methyl-8-(1-methyl-1H-1,2,3-triazol-5-yl)-2-oxo-2,3-dihydro-1H-imidazo[4,5-d]pyrrolo[1,2-b]pyridazin-1-yl)cyclopentyl)carbamate

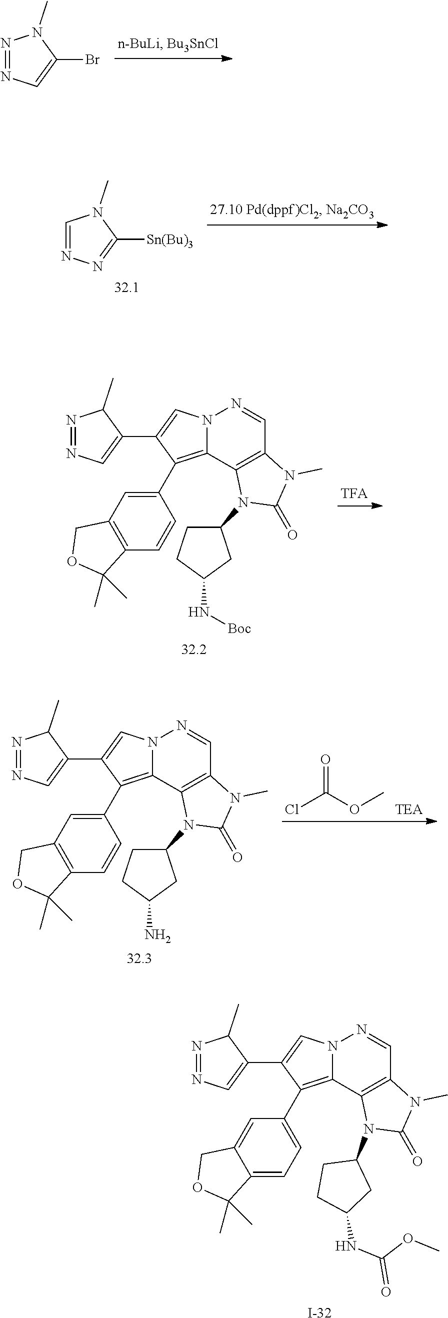

Synthesis of compound 32.1. Compound 32.1 was prepared from compound 5-bromo-1-methyl-1H-1,2,3-triazole following the procedure described in the synthesis of compound 19.1. The product was purified by flash column chromatography on silica gel (Combiflash®, 10% ethyl acetate in n-hexane). m/z: 374.6 [M+H]⁺.

Synthesis of compound 32.2. Compound 32.2 was prepared from compound 27.10 and 32.1 following the procedure described in the synthesis of compound 1.13. The product was purified by flash column chromatography on silica gel (Combiflash®, 49% ethyl acetate in hexane). m/z-599.30 [M+H]⁺.

Synthesis of compound 32.3. Compound 32.3 was prepared from compound 32.2 following the procedure described in the synthesis of compound 1.14. The product was used in the next step without purification. MS (ES): m/z 498.59 [M+H]⁺.

Synthesis of compound I-32. Compound I-32 was prepared from compound 32.3 following the procedure described in the synthesis of compound I-1. The product was purified by flash column chromatography on silica gel (Combiflash®, 50% ethyl acetate in hexane). MS (ES): m/z 557.15 [M+H]⁺, ¹H NMR (DMSO-d₆, 400 MHz): δ 8.57 (s, 1H), 8.37 (s, 1H), 7.33 (m, 1H), 7.24 (m, 2H), 7.10-7.05 (m, 1H), 7.01 (m, 0.6H) 6.84 (m, 0.6H), 4.99 (m, 2H), 4.15-3.86 (m, 4H), 3.48 (s, 3H), 3.40-3.37 (m, 4H), 2.05-2.02 (m, 2H), 1.86-1.83 (m, 2H), 1.57 (s, 6H), 1.47-1.44 (m, 2H).

### Example I-33: (S)-tetrahydro-2H-pyran-3-yl ((1R,3R)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

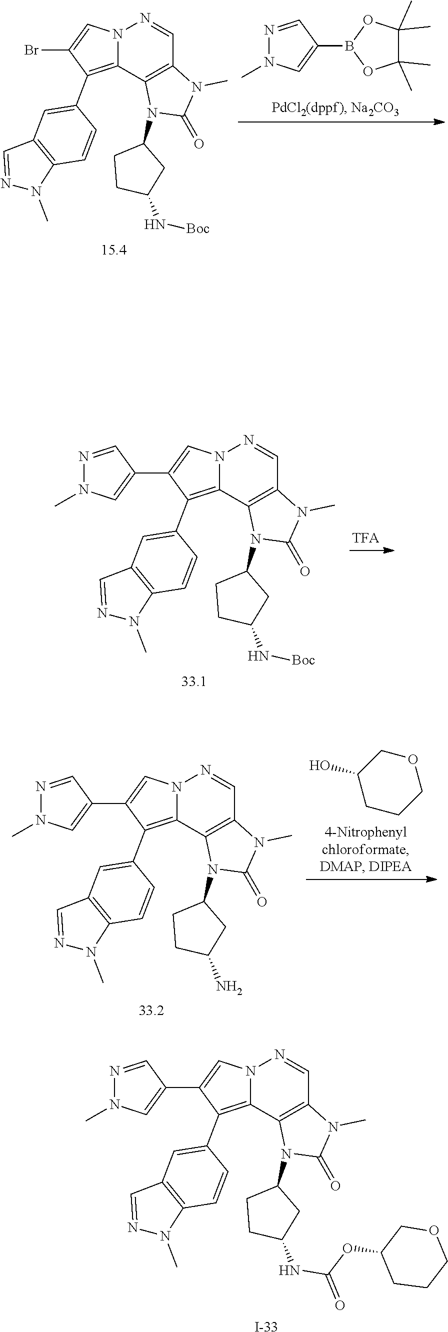

Synthesis of compound 33.1. Compound 33.1 was prepared from compound 15.4 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (Combiflash®, 2.6% methanol in DCM). MS(ES): m/z 582.5 [M+H]⁺.

Synthesis of compound 33.2. Compound 33.2 was prepared from compound 33.1 following the procedure described in the synthesis of compound 1.14. The product was used in the next step without purification. MS(ES): m/z 482.3 [M+H]⁺.

Synthesis of I-33. To a solution of (S)-tetrahydro-2H-pyran-3-ol (0.038 g, 0.373 mmol, 1.0 equiv), 4-dimethylaminopyridine (0.068 g, 0.558 mmol, 1.5 equiv) and N,N-diisopropylethylamine (0.144 g, 1.11 mmol, 3.0 equiv) in DCM (4.0 mL) at 0° C. was added 4-nitrophenyl chloroformate (0.112 g, 0.558 mmol, 1 equiv) dropwise and stirred for 20 min. It was concentrated under reduced pressure. The residue was dissolved in DMF (2 mL) and was added N,N-diisopropylethylamine (0.288 g, 2.23 mmol, 6.0 equiv) and 33.2 (0.060 g, 0.125 mmol, 0.33 equiv). The reaction mixture was stirred at 65° C. for 2 h. It was poured into water and extracted with ethyl acetate. The combined organic layers were washed with brine, dried over anhydrous sodium sulfate, filtered and concentrated under reduced pressure. The residue was purified by flash column chromatography on silica gel (Combiflash®, 2.7% methanol in DCM) to afford I-33 (0.026 g, 34%). MS(ES): m/z 610.25 [M+H]⁺, ¹H-NMR (400 MHz, DMSO-d₆): δ 8.32 (m, 1H), 8.08-8.05 (m, 2H), 7.75-7.72 (m, 2H), 7.40 (d, J=7.6 Hz, 1H), 7.24 (s, 1H), 7.02 (s, 1H), 6.23-5.84 (m, 1H), 4.46 (s, 1H), 4.14 (s, 3H), 3.91-3.89 (m, 1H), 3.70-3.66 (m, 1H), 3.61 (s, 3H), 3.60-3.56 (m, 2H), 3.55-3.52 (m, 2H), 3.49 (s, 3H), 2.09 (s, 1H), 1.90-1.87 (m, 2H), 1.78-1.74 (m, 2H), 1.61-1.50 (m, 4H), 0.42-0.01 (m, 1H).

### Example I-34: methyl ((1R,3R)-3-(8-(1-(2-hydroxy-2-methylpropyl)-1H-pyrazol-4-yl)-3-methyl-9-(1-methyl-1H-indazol-5-yl)-2-oxo-2,3-dihydro-1H-imidazo[4,5-d]pyrrolo[1,2-b]pyridazin-1-yl)cyclopentyl)carbamate

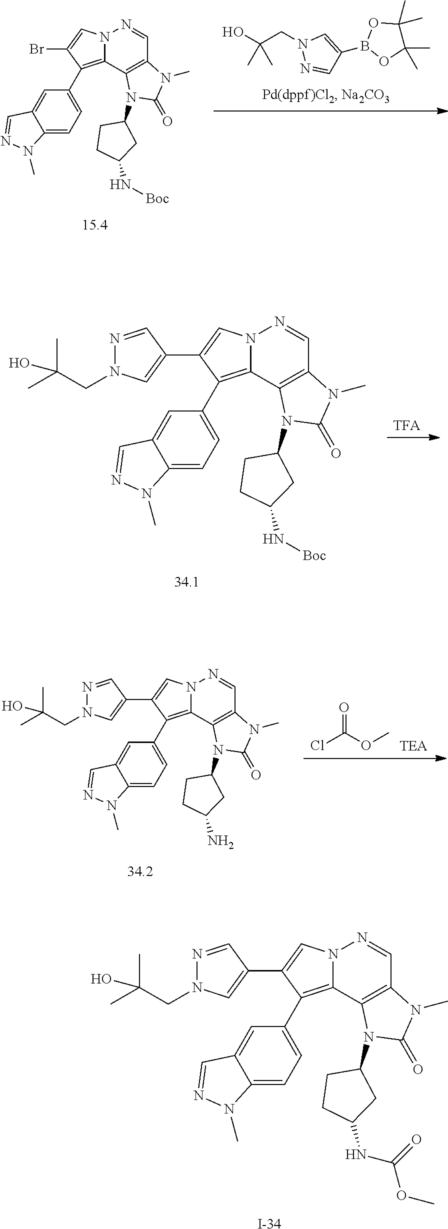

Synthesis of compound 34.1. Compound 34.1 was prepared from compound 15.4 and 2-methyl-1-(4-(4,4,5,5-tetramethyl-1,3,2-dioxaborolan-2-yl)-1H-pyrazol-1-yl)propan-2-ol following the procedure described in the synthesis of compound 1.13. The product was purified by flash column chromatography on silica gel (2.8% methanol in DCM). MS(ES): m/z 640.4 [M+H]⁺.

Synthesis of compound 34.2. Compound 34.2 was prepared from compound 34.1 following the procedure described in the synthesis of compound 1.14. The product was used in the next step without purification. MS(ES): m/z 540.6 [M+H]⁺.

Synthesis of I-34. Compound I-34 was prepared from compound 34.2 following the procedure described in the synthesis of compound I-1. The product was purified by silica gel flash column chromatography on silica gel (3.9% methanol in DCM). MS(ES): m/z 598.00 [M+H]⁺, 1H-NMR (400 MHz, DMSO-d₆): δ 8.38 (s, 1H), 8.17 (d, J=3.6 Hz, 1H), 8.05 (d, J=3.2 Hz, 1H), 7.81-7.75 (m, 2H), 7.41-7.37 (m, 1H), 7.20-7.01 (m, 2H), 6.68-6.34 (m, 1H), 4.54 (s, 1H), 4.14 (s, 3H), 3.98-3.79 (m, 3H), 3.55 (s, 3H), 3.45 (s, 1H), 3.33 (s, 3H), 2.00 (bs, 1H), 1.74 (bs, 1H), 1.50 (bs, 1H), 1.35 (bs, 1H), 1.06 (bs, 1H), 0.91 (s, 6H), 0.34 (bs, 1H).

### Example I-35: methyl ((1R,3R)-3-(8-(1-(2-methoxyethyl)-1H-pyrazol-4-yl)-3-methyl-9-(1-methyl-1H-indazol-5-yl)-2-oxo-2,3-dihydro-1H-imidazo[4,5-d]pyrrolo[1,2-b]pyridazin-1-yl)cyclopentyl)carbamate

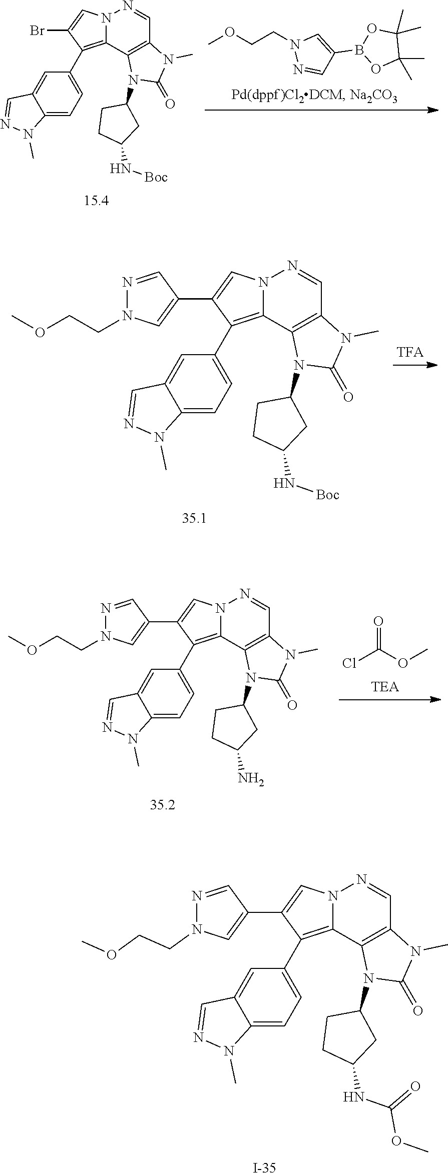

Synthesis of compound 35.1. Compound 35.1 was prepared from compound 15.4 and 1-(2-methoxyethyl)-4-(4,4,5,5-tetramethyl-1,3,2-dioxaborolan-2-yl)-1H-pyrazole following the procedure described in the synthesis of compound 1.13. The product was purified by flash column chromatography on silica gel (2.8% methanol in DCM). MS(ES): m/z 626.3 [M+H]⁺.

Synthesis of compound 35.2. Compound 35.2 was prepared from compound 35.1 following the procedure described in the synthesis of compound 1.14. The product was used in the next step without purification. MS(ES): m/z 526.4 [M+H]⁺.

Synthesis of I-35. Compound I-35 was prepared from compound 35.2 following the procedure described in the synthesis of compound I-1. The product was purified by silica gel flash column chromatography on silica gel (3.9% methanol in DCM). MS(ES): m/z 584.52 [M+H]⁺, ¹H-NMR (400 MHz, DMSO-d₆): δ 8.38 (s, 1H), 8.17 (s, 1H), 8.06 (d, J=6.4 Hz, 1H), 7.81-7.76 (m, 2H), 7.41-7.37 (m, 1H), 7.21-7.03 (m, 2H), 6.67-6.31 (m, 1H), 4.22 (s, 3H), 4.14-4.12 (m, 2H), 3.87 (bs, 1H), 3.48 (s, 4H), 3.34 (bs, 1H), 3.34 (s, 3H), 3.03 (s, 3H), 2.00-1.98 (m, 1H), 1.72-7.71 (m, 1H), 1.50 (bs, 1H), 1.23-1.08 (m, 2H), 0.85 (bs, 1H), 0.36 (bs, 1H).

### Example I-36: methyl ((1R,3R)-3-(8-(2,3-dihydropyrazolo[5,1-b]oxazol-7-yl)-9-(1,1-dimethyl-1,3-dihydroisobenzofuran-5-yl)-3-methyl-2-oxo-2,3-dihydro-1H-imidazo[4,5-d]pyrrolo[1,2-b]pyridazin-1-yl)cyclopentyl)carbamate

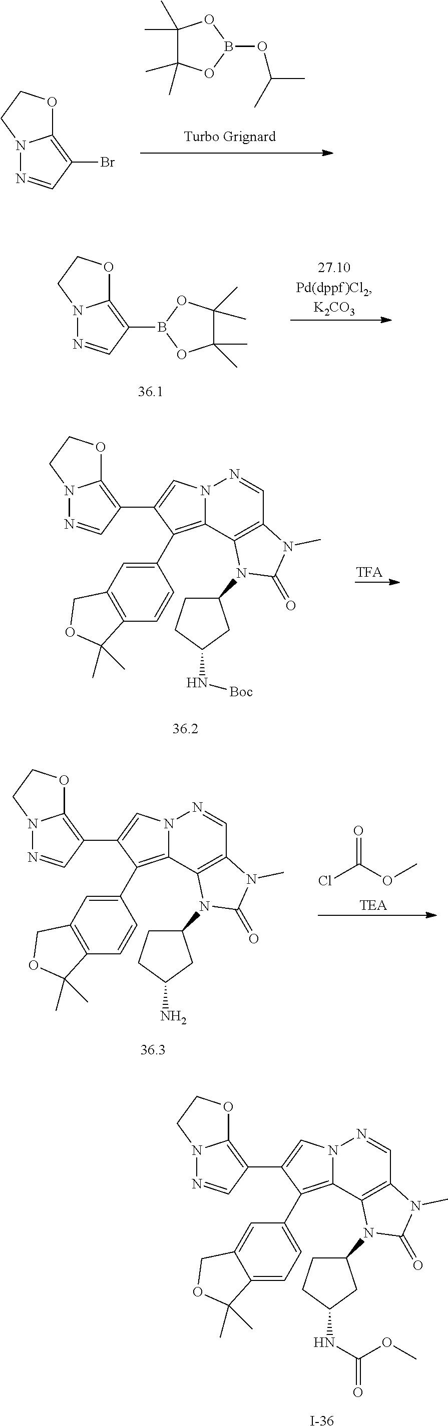

Synthesis of of compound 36.1. To a solution of 7-bromo-2,3-dihydropyrazolo[5,1-b]oxazole (5 g, 26.45 mmol, 1.0 equiv) in THF (50 mL) was added isopropyl magnesium chloride lithium chloride complex (1.5 M, 26 mL, 39.67 mmol, 1.5 equiv) at 0° C. dropwise to the reaction mixture and stirred at 0° C. for 1 h. To the reaction mixture was added 2-isopropoxy-4,4,5,5-tetramethyl-1,3,2-dioxaborolane (9.61 g, 52.9 mmol, 2 equiv) and stirred at room temperature for 2 h. It was quenched with saturated ammonium chloride solution and extracted with ethyl acetate. The combined organic layers were washed with brine, dried over anhydrous sodium sulfate, filtered and concentrated under reduced pressure. The residue was purified by flash column chromatography on silica gel (Combiflash®, 50% hexane in ethyl acetate) to afford 36.1 (3.8 g, 61%). MS(ES): m/z 237.68 [M+H]⁺.

Synthesis of compound 36.2. A mixture of 27.10 (0.2 g, 0.335 mmol, 1 equiv), 36.1 (0.095 g, 0.402 mmol, 1.2 equiv) and potassium carbonate (0.138 g, 1.0 mmol, 3 equiv) in 1,4-dioxane:water (8:2, 10 mL) was degassed by bubbling through a stream of argon for 20 min. 1,1′-Bis(diphenylphosphino)ferrocene]dichloropalladium(II) (0.024 g, 0.033 mmol, 0.1 equiv) was added to reaction mixture and stirred at 100° C. for 4 h. It was cooled to room temperature and transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 1.8% methanol in DCM) to afford 36.2 (0.07 g, 33%). MS (ES): m/z 626.3 [M+H]⁺.

Synthesis of compound 36.3. Compound 36.3 was prepared from compound 36.2 following the procedure described in the synthesis of compound 1.14. The product was used in the next step without purification. MS(ES): m/z 526.26 [M+H]⁺.

Synthesis of compound I-36. Compound I-36 was prepared from compound 36.3 following the procedure described in the synthesis of compound I-1. The product was purified by flash column chromatography on silica gel (Combiflash®, 2.6% methanol in DCM). MS(ES): m/z 584.26 [M+H]⁺, ¹H NMR (DMSO-d₆, 400 MHz): δ 8.37 (s, 1H), 7.80 (s, 1H), δ 7.38-7.30 (m, 2H), 7.24 (s, 1H), 7.01 (m, 1H), 7.00 (m, 1H), 6.43-6.36 (m, 1H), 5.16-5.10 (m, 1H), 5.09-5.06 (m, 2H), 5.01-4.99 (m, 1H), 4.21 (t, J=8 Hz, 2H), 4.04-4.02 (m, 1H), 3.92-3.86 (m, 1H), 3.48 (s, 3H), 3.42 (s, 3H) 2.03-2.00 (m, 1H), 1.90-1.84 (m, 3H) 1.51-1.48 (m, 6H) 1.35-1.30 (bs, 1H).

### Example I-37-a and I-37-b: 2-(((1R,3S)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)oxy)acetonitrile and 2-(((1S,3R)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)oxy)acetonitrile

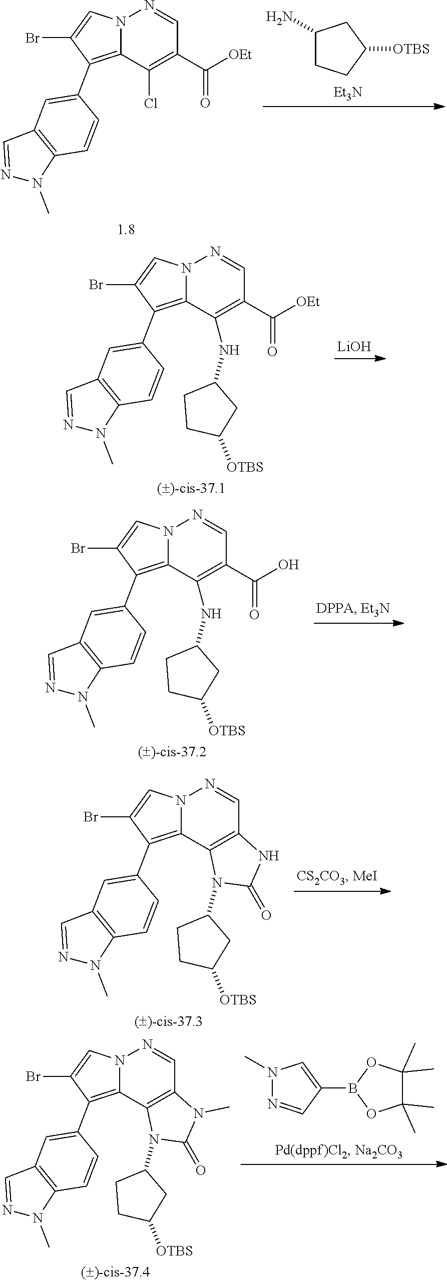

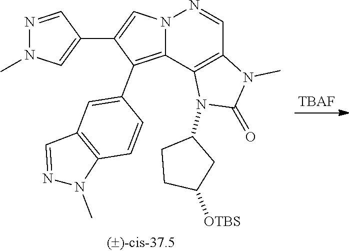

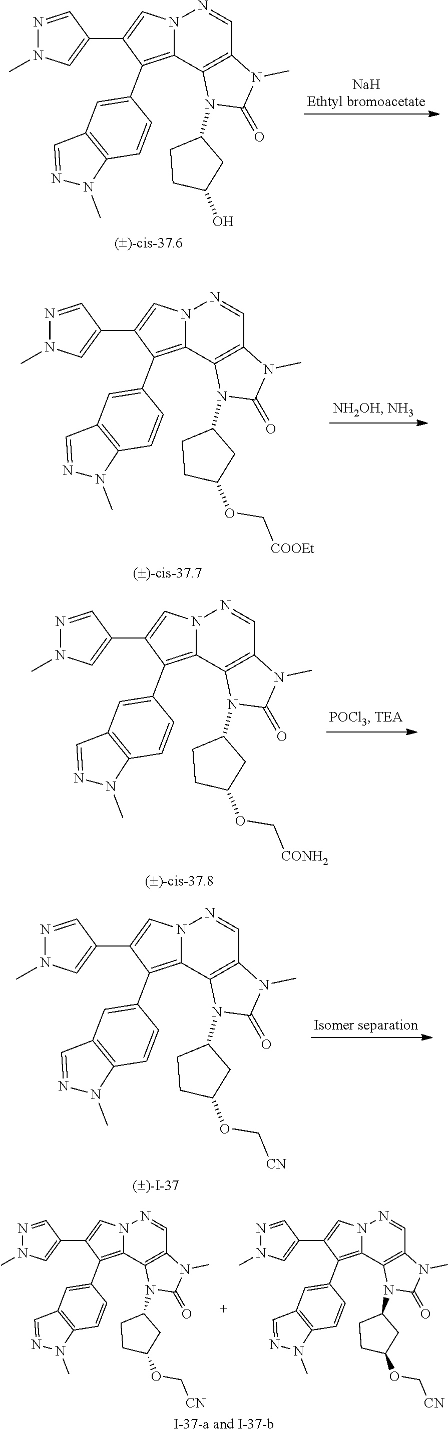

Synthesis of compound (±)-cis-37.1. Compound (±)-cis-37.1 was prepared from compound 1.8 and (1S,3R)-3-((tert-butyldimethylsilyl)oxy)cyclopentan-1-amine following the procedure described in the synthesis of compound 1.9. The product was purified by flash column chromatography on silica gel (Combiflash®, 0-30% ethyl acetate in hexane). MS(ES): m/z 613.3 and 615.3 [M+H]⁺.

Synthesis of compound (±)-cis-37.2. Compound (±)-cis-37.2 was prepared from compound (±)-cis-37.1 following the procedure described in the synthesis of compound 1.10. The product was triturated with pentane, dried under vacuum and used in the next step without purification. MS(ES): m/z 584.3 and 586 [M+H]⁺.

Synthesis of compound (±)-cis-37.3. Compound (±)-cis-37.3 was prepared from compound (±)-cis-37.2 following the procedure described in the synthesis of compound 1.11. The product was purified by flash column chromatography on silica gel (Combiflash®, 0-3% methanol in DCM). MS(ES): m/z 582 and 584 [M+H]⁺.

Synthesis of compound (±)-cis-37.4. Compound (±)-cis-37.4 was prepared from compound (±)-cis-37.3 following the procedure described in the synthesis of compound 1.12. The product was purified by flash column chromatography on silica gel (0-3% methanol in DCM). MS(ES): m/z 596 and 598 [M+H]⁺.

Synthesis of compound (±)-cis-37.5. Compound (±)-cis-37.5 was prepared from compound (±)-cis-37.4 following the procedure described in the synthesis of compound 1.13. The product was purified by flash column chromatography on silica gel (Combiflash®, 2.2% methanol in DCM). MS(ES): m/z 597.1 [M+H]⁺.

Synthesis of compound (±)-cis-37.6. To a solution of (±)-cis-37.5 (1.2 g, 2.01 mmol, 1.0 equiv) in THF (20.0 mL) at 0° C. was added TBAF (12 mL) dropwise and stirred for 1 h at room temperature. The reaction mixture was concentrated under reduced pressure. The residue was purified by flash column chromatography on silica gel (Combiflash®, 2.2% methanol in DCM) to afford (±)-cis-37.6 (0.640 g, 66%). MS(ES): m/z 484 [M+H]⁺.

Synthesis of compound (±)-cis-37.7. To a solution of (±)-cis-37.6 (0.640 g, 1.32 mmol, 1.0 equiv) in THF (280 mL) was added sodium hydride (60% dispersion in mineral oil, 0.079 g, 1.98 mmol, 1.5 equiv) in small portions at 0° C. The mixture was stirred at 30 min and added ethyl bromoacetate (0.331 g, 1.98 mmol, 1.5 equiv). The reaction mixture was stirred at 60° C. for 4 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (2.2% methanol in DCM) to afford (±)-cis-37.7 (0.450 g, 60%). MS(ES): m/z 569.23 [M+H]⁺.

Synthesis of compound (±)-cis-37.8. A solution of (±)-cis-37.7 (0.450 g, 0.79 mmol, 1.0 equiv) in a mixture of methanolic ammonia (0.5 mL) and aqueous ammonia (0.5 mL) was stirred at 80° C. for 30 min. The reaction mixture was concentrated under reduced pressure. The residue was purified by flash column chromatography on silica gel (Combiflash®, 3.2% methanol in DCM) to afford (±)-cis-37.8 (0.230 g, 53.8%). MS(ES): m/z 540 [M+H]⁺.

Synthesis of compound (±)—I-37. To a solution of (±)-cis-37.8 (0.230 g, 0.426 mmol, 1.0 equiv) and triethylamine (0.215 g, 2.13 mmol, 5.0 equiv) in DCM (5 mL) at 0° C. was added POCl₃ (0.197 g, 1.28 mmol, 3.0 equiv) and stirred for 30 min. The reaction mixture was poured into ice-water and quenched with a saturated aqueous solution of NaHCO₃ and extracted with DCM. The combined organic layers were washed with brine, dried over anhydrous sodium sulfate, filtered and concentrated under reduced pressure. The residue was purified by flash column chromatography on silica gel (2.9% methanol in DCM) to afford (±)—I-37 (0.060 g, 27%). MS(ES): m/z 522.4 [M+H]⁺.

The racemate was separated by chiral HPLC (Column: CHIRALPAK OX-H (250*30 mm), 5 m; Mobile Phase: (A) 0.1% NH₃ in n-hexane (B) 0.1% NH₃ in IPA: ACN (70:30) Flow: 35 mL/min) to afford first eluting fraction (I-37-a: MS(ES): m/z 522.4 [M+H]⁺, ¹H NMR (DMSO-d₆, 400 MHz): δ 8.40 (s, 1H), 8.16 (s, 1H), 8.12-8.10 (d, J=7.2 Hz, 1H), 7.80-7.78 (d, J=7.2 Hz, 2H), 7.42-7.40 (m, 1H), 7.25-7.24 (d, J=7.2 Hz, 1H), 7.04-7.01 (d, J=8.4 Hz, 1H), 6.84 (m, 1H), 4.26 (s, 1H), 4.16 (s, 3H), 4.09 (m, 1H), 3.70 (s, 3H), 3.38 (s, 3H), 2.20-1.98 (m, 4H), 1.52-1.20 (m, 4H), and second eluting fraction (I-37-b: MS(ES): m/z 522.4 [M+H]⁺, ¹H NMR (DMSO-d₆, 400 MHz): δ 8.40 (s, 1H), 8.16 (s, 1H), 8.12-8.10 (d, J=7.2 Hz, 1H), 7.80-7.78 (d, J=7.2 Hz, 2H), 7.42-7.40 (m, 1H), 7.25-7.24 (d, J=7.2 Hz, 1H), 7.04-7.01 (d, J=8.4 Hz, 1H), 6.84-6.83 (m, 1H) 4.28 (s, 1H), 4.15 (s, 3H), 4.09 (m, 1H), 3.70 (s, 3H), 3.378 (s, 3H), 2.22-1.95 (m, 4H), 1.55-1.21 (m, 4H).

### Example I-38: methyl ((1R,3R)-3-(3-methyl-9-(1-methyl-1H-indazol-5-yl)-2-oxo-8-(thiazol-5-yl)-2,3-dihydro-1H-imidazo[4,5-d]pyrrolo[1,2-b]pyridazin-1-yl)cyclopentyl)carbamate

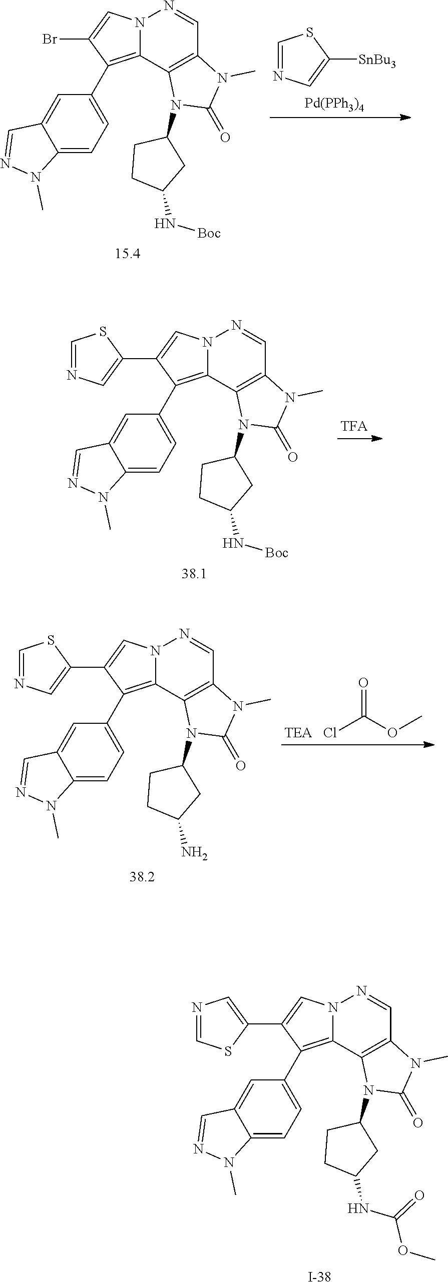

Synthesis of compound 38.1. A mixture of 15.4 (0.08 g, 0.13 mmol, 1.0 equiv), 5-(tributylstannyl)thiazole (0.200 g, 0.55 mmol, 4.0 equiv) and tetrakis(triphenylphosphine)palladium (0) (0.016 g, 0.013 mmol, 0.1 equiv) in 1,4-dioxane (5 mL) was degassed by bubbling through a stream of argon for 10 min. The reaction mixture was stirred at 140° C. for 1 h. It was cooled to room temperature, filtered through a pad of Celite® and rinsed with ethyl acetate. The filtrate was transferred into water and extracted with ethyl acetate. The combined organic layers were washed with brine, dried over anhydrous sodium sulfate, filtered and concentrated under reduced pressure. The residue was purified by flash column chromatography on silica gel (3.0% methanol in DCM) to afford 38.1 (0.06 g, 74.5%). MS(ES): m/z 585.6 [M+H]⁺.

Synthesis of compound 38.2. Compound 38.2 was prepared from compound 38.1 following the procedure described in the synthesis of compound 1.14. The product was used in the next step without purification. MS(ES): m/z 485.6 [M+H]⁺.

Synthesis of I-38. Compound I-38 was prepared from compound 38.2 following the procedure described in the synthesis of compound I-1. The product was purified by silica gel flash column chromatography on silica gel (3.2% methanol in DCM). MS(ES): m/z 543.3 [M+H]⁺, ¹H-NMR (400 MHz, DMSO-d₆): δ 8.74 (s, 1H), 8.49 (d, J=1.2 Hz, 1H), 8.45 (d, J=2.0 Hz, 1H), 8.08 (d, J=4.8 Hz, 1H), 7.91-7.87 (m, 1H), 7.81-7.77 (m, 2H), 7.42-7.38 (m, 1H), 6.70-6.69 (m, 0.5H), 6.38-6.37 (m, 0.5H), 4.14 (s, 3H), 3.85 (m, 1H), 3.51 (s, 3H), 3.49-3.47 (m, 1H), 3.35 (s, 3H), 2.10-1.90 (m, 1H), 1.80-1.70 (m, 1H), 1.60-1.40 (m, 1H), 1.35-1.33 (m, 1H), 1.10-1.08 (m, 1H), 0.87 (m, 1H).

### Example I-40: methyl ((1R,3R)-3-(9-(2,3-dihydrobenzo[b][1,4]dioxin-6-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

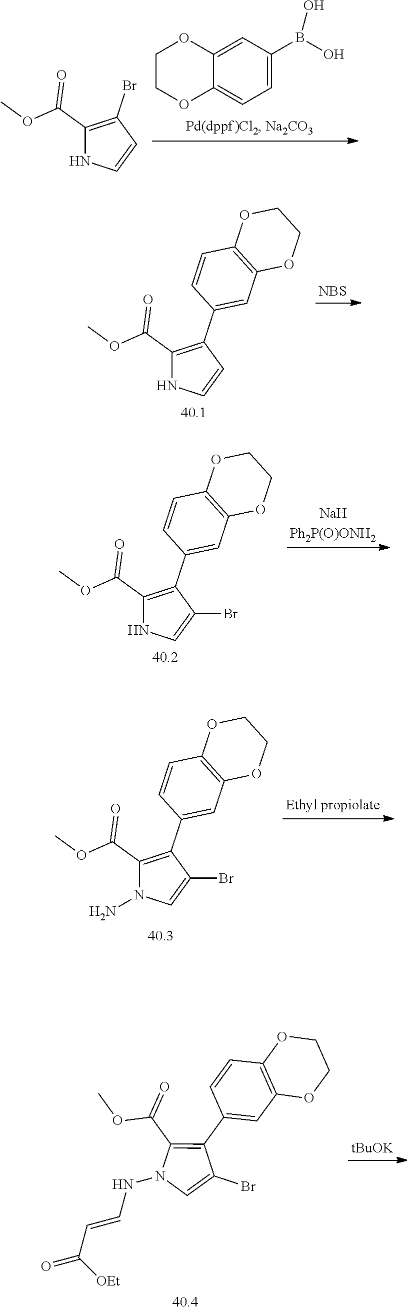

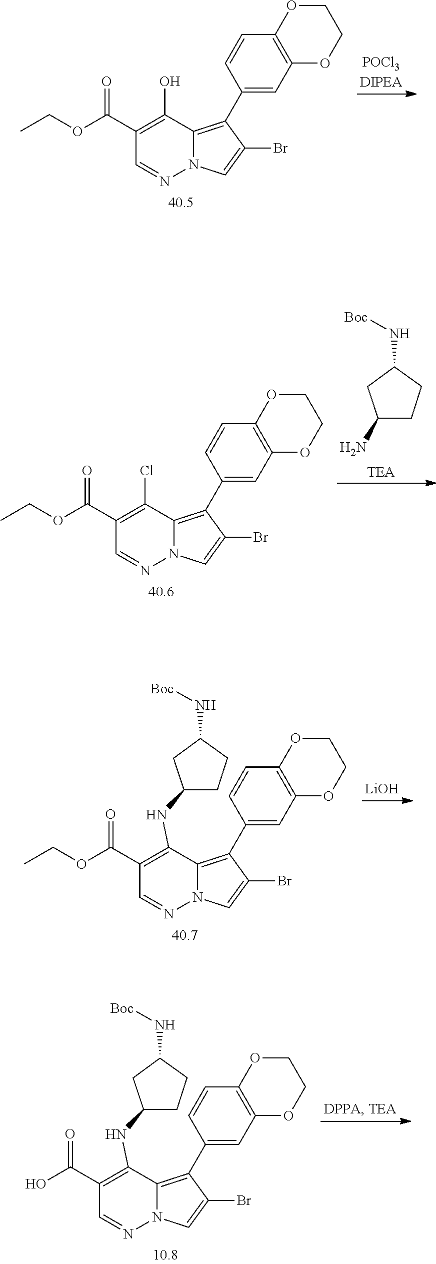

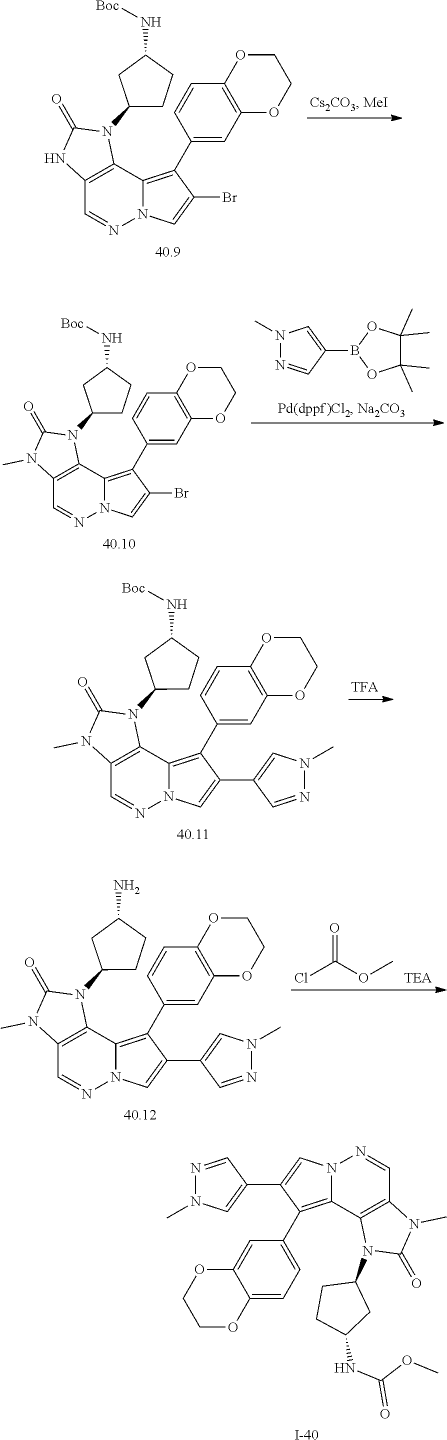

Synthesis of compound 40.1. Compound 40.1 was prepared from compound methyl 3-bromo-1H-pyrrole-2-carboxylate and (2,3-dihydrobenzo[b][1,4]dioxin-6-yl)boronic acid following the procedure described in the synthesis of compound 1.2. The product was purified by flash column chromatography on silica gel (30% ethyl acetate in hexane). MS(ES): m/z 260.2 [M+H]⁺.

Synthesis of compound 40.2. Compound 40.2 was prepared from compound 40.1 following the procedure described in the synthesis of compound 1.4. The product was purified by flash column chromatography on silica gel (25% ethyl acetate in hexane). MS(ES): m/z 338.1 [M+H]⁺.

Synthesis of compound 40.3. Compound 40.3 was prepared from compound 40.2 following the procedure described in the synthesis of compound 1.5. The product was purified by flash column chromatography on silica gel (30% ethyl acetate in hexane. MS(ES): m/z 353.10 [M+H]⁺.

Synthesis of compound 40.4. Compound 40.4 was prepared from compound 40.3 following the procedure described in the synthesis of compound 1.6. The product was purified by flash column chromatography on silica gel (40% ethyl acetate in hexane). MS(ES): m/z 451.2 [M+H]⁺.

Synthesis of compound 40.5. Compound 40.5 was prepared from compound 40.4 following the procedure described in the synthesis of compound 1.7. MS(ES): m/z 420.23 [M+H]⁺.

Synthesis of compound 40.6. Compound 40.6 was prepared from compound 40.5 following the procedure described in the synthesis of compound 1.8. The product was purified by flash column chromatography on silica gel (30% ethyl acetate in hexane). MS(ES): m/z 438.6 [M+H]⁺.

Synthesis of compound 40.7. Compound 40.7 was prepared from compound 40.6 and tert-butyl ((1R,3R)-3-aminocyclopentyl)carbamate following the procedure described in the synthesis of compound 1.9. The product was purified by flash column chromatography on silica gel (Combiflash®, 50% ethyl acetate in hexane). MS(ES): m/z 602.5 [M+H]⁺.

Synthesis of compound 40.8. Compound 40.8 was prepared from compound 40.7 following the procedure described in the synthesis of compound 1.10. The product was triturated with pentane and used in the next step without purification. MS(ES): m/z 574.4 [M+H]⁺.

Synthesis of compound 40.9. Compound 40.9 was prepared from compound 40.8 following the procedure described in the synthesis of compound 1.11. The product was purified by flash column chromatography on silica gel (Combiflash®, 1.8% methanol in DCM). MS(ES): m/z 571.4 [M+H]⁺.

Synthesis of compound 40.10. Compound 40.10 was prepared from compound 40.9 following the procedure described in the synthesis of compound 1.12. The product was purified by flash column chromatography on silica gel (1.4% methanol in DCM). MS(ES): m/z 585.4 [M+H]⁺.

Synthesis of compound 40.11. Compound 40.11 was prepared from compound 40.10 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (Combiflash®, 0-3.2% methanol in DCM). MS(ES): m/z 586.6 [M+H]⁺.

Synthesis of compound 40.12. Compound 40.12 was prepared from compound 40.11 following the procedure described in the synthesis of compound 1.14. The product was triturated with pentane and dried under reduced pressure. It was used in the next step without purification. MS(ES): m/z 486.5 [M+H]⁺.

Synthesis of I-40. Compound I-40 was prepared from compound 40.12 following the procedure described in the synthesis of compound I-1. The product was purified by flash column chromatography on silica gel (Combiflash®, 0-2.6% methanol in DCM). MS(ES): m/z 544 [M+H]⁺, ¹H NMR (DMSO-d₆, 400 MHz): δ 8.36 (s, 1H), 8.09 (s, 1H), 7.31 (s, 1H), 7.12-7.11 (d, J=4 Hz, 1H), 7.00-6.98 (m, 2H), 6.84-6.82 (m, 2H), 1.34-4.32 (m, 3H), 4.29-4.25 (m, 1H), 4.05 (m, 2H), 3.92-3.89 (m, 2H), 3.73 (s, 3H), 3.50 (s, 3H), 3.42 (s, 3H), 2.12-2.08 (m, 2H), 1.99-1.86 (m, 2H), 1.54-1.50 (m, 2H).

### Example I-41: 3-methyl-9-(1-methyl-1H-indazol-5-yl)-8-(1-methyl-1H-pyrazol-4-yl)-1-((1S,3S)-3-(2-oxopyrrolidin-1-yl)cyclopentyl)-1,3-dihydro-2H-imidazo[4,5-d]pyrrolo[1,2-b]pyridazin-2-one

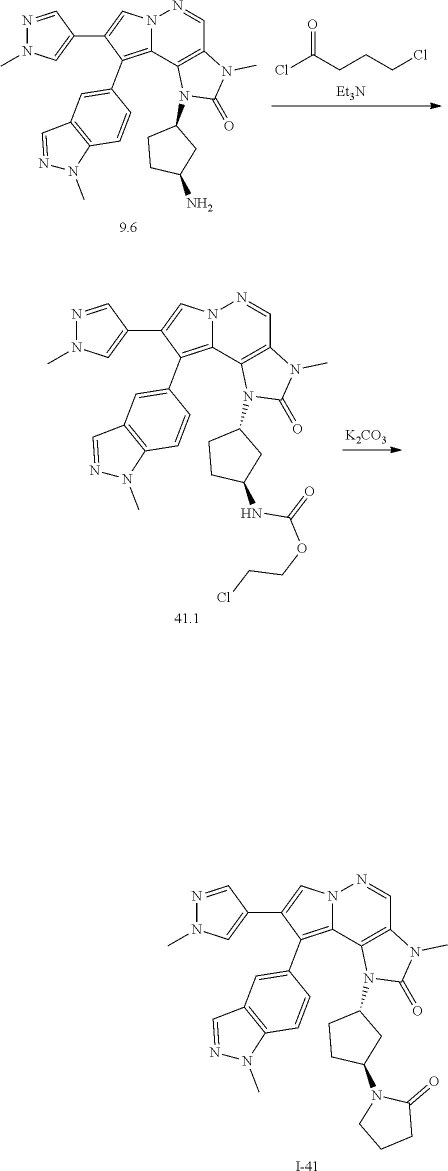

Synthesis of compound 41.1. To a solution of 9.6 (0.12 g, 0.24 mmol, 1.0 equiv) and triethylamine (0.1 mL, 0.74 mmol, 3.0 equiv) in THF (4 mL) was added 4-chlorobutanoyl chloride (0.067 g, 0.48 mmol, 2.0 equiv) dropwise at 0° C. and stirred for 10 min. It was transferred into water and extracted with ethyl acetate. The combined organic layers were washed with brine, dried over anhydrous sodium sulfate, filtered and concentrated under reduced pressure. The residue was purified by flash column chromatography on silica gel (Combiflash®, 3.2% methanol in DCM) to afford 41.1 (0.085 g, 58%). MS(ES): m/z 587.3 [M+H]⁺.

Synthesis of I-41. A solution of 41.1 (0.085 g, 0.14 mmol, 1.0 equiv) and potassium carbonate (0.060 g, 0.43 mmol, 3 equiv) in dimethyl sulfoxide (3 mL) was stirred at 70° C. for 1 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 3.4% methanol in DCM) to afford I-41 (0.030 g, 38%). MS(ES): m/z 550.51 [M+H]⁺, ¹H-NMR (400 MHz, DMSO-d₆): δ 8.40 (s, 1H), 8.14 (dd, J=1.20, 17.2 Hz, 2H), 7.86-7.78 (m, 2H), 7.50-7.38 (m, 1H), 7.25 (d, J=3.6 Hz, 1H), 7.01-6.97 (m, 1H), 4.42 (s, 1H), 4.13 (s, 3H), 3.65 (s, 3H), 3.38 (s, 3H), 2.89-2.67 (m, 1H), 2.32-2.28 (m, 1H), 2.14-2.11 (m, 2H), 1.99-1.86 (m, 3H), 1.68-1.64 (m, 2H), 1.43 (bs, 1H), 1.06-1.02 (m, 1H), 0.50 (s, 1H), −0.03 (s, 1H).

### Example I-42: 1-((3S,5R)-5-(hydroxymethyl)tetrahydrofuran-3-yl)-3-methyl-9-(1-methyl-1H-indazol-5-yl)-8-(1-methyl-1H-pyrazol-4-yl)-1,3-dihydro-2H-imidazo[4,5-d]pyrrolo[1,2-b]pyridazin-2-one

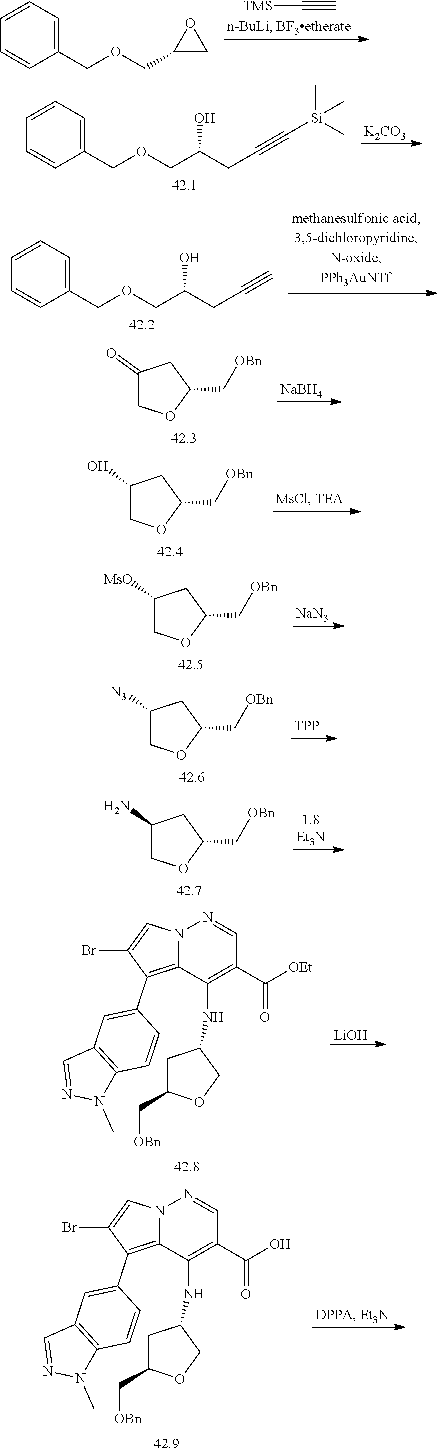

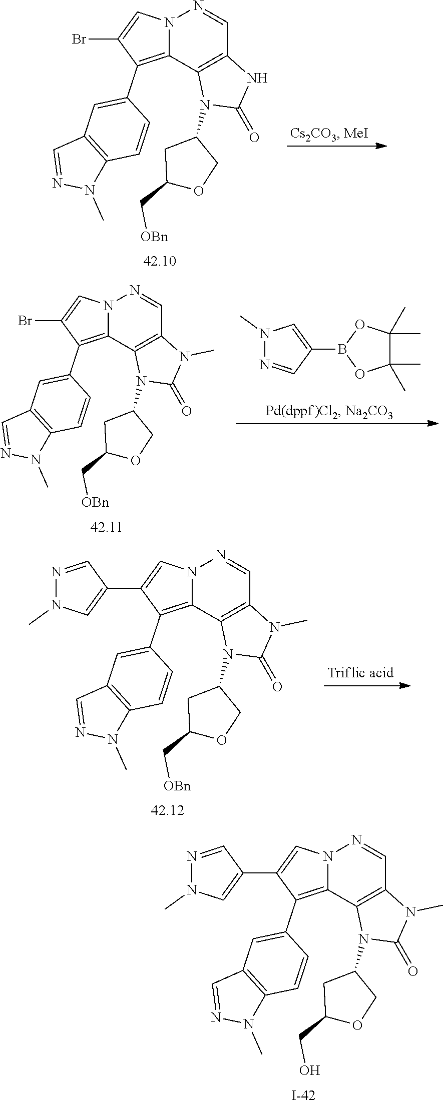

Synthesis of compound 42.1. To a solution of (R)-2-((benzyloxy)methyl)oxirane (7.18 g, 73.08 mmol, 1.5 equiv) in THF (100 mL) at −78° C. was added n-butyllithium in hexane (2.5 M, 31.1 mL, 78.04 mmol, 1.6 equiv) dropwise. The reaction mixture was allowed to warm at −30° C. and stirred for 30 min. It was cooled to −78° C. and added boron trifluoride-diethyl ether (11.0 g, 78.04 mmol, 1.6 equiv) dropwise followed by slowly addition of trimethylsilylacetylene (8.0 g, 48.72 mmol, 1.0 equiv) in THF (80 mL). The reaction mixture was stirred for 2 h. It was quenched with a saturated solution of ammonium chloride and extracted with ethyl acetate. The combined organic layers were washed with brine, dried over anhydrous sodium sulfate, filtered and concentrated under reduced pressure. The residue was purified by flash column chromatography on silica gel (10% ethyl acetate in hexane) to afford 42.1 (7.0 g, 55%). MS(ES): m/z 263.45 [M+H]⁺.

Synthesis of compound 42.2. A mixture of 42.1 (7.0 g, 26.88 mmol, 1.0 equiv) and potassium carbonate (11.0 g, 80.15 mmol, 3.0) in methanol (70 mL) was stirred at room temperature for 2 h. It was transferred to ice-water and extracted with ethyl acetate. The combined organic layers were washed with brine, dried over anhydrous sodium sulfate, filtered and concentrated under reduced pressure. The residue was purified by flash column chromatography on silica gel (15% ethyl acetate in hexane) to afford 42.2 (4.8 g, 95%). MS(ES): m/z 191.4 [M+H]⁺.

Synthesis of compound 42.3. To a solution of 42.2 (2.0 g, 10.51 mmol, 1.0 equiv) in 1,2-dichloroethane (30 mL) was added 3,5-dichloropyridine-N-oxide (3.43 g, 21.05 mmol, 2 equiv) followed by methanesulfonic acid (1.2 g, 12.63 mmol, 1.2 equiv) and triphenylphosphinegold(I) bis(trifluoromethanesulfonyl)imidate (0.38 g, 0.52 mmol, 0.05 equiv) at room temperature. The reaction mixture was stirred for 16 h. It was concentrated under reduce pressure. The residue was purified by flash column chromatography on silica gel (16% ethyl acetate in hexane) to afford 42.3 (0.65 g, 30%). ¹H NMR (CDCl₃, 400 MHz): δ 7.37-7.27 (m, 5H), 4.71-4.51 (m, 2H), 4.19-4.15 (d, J=16 Hz, 1H), 3.94-3.89 (d, J=16 Hz, 1H), 3.76-3.74 (d, J=8 Hz, 1H), 3.62-3.60 (d, J=8 Hz, 1H), 2.57-2.43 (m, 2H).

Synthesis of compound 42.4. To a solution of 42.3 (0.65 g, 3.15 mmol, 1.0 equiv) in methanol (15 mL) was added sodium borohydride (0.299 g, 7.88 mmol, 2.5 equiv) in small portions at 0° C. The reaction mixture was stirred at room temperature for 16 h. It was transferred to ice-water and extracted with ethyl acetate. The combined organic layers were washed with brine, dried over anhydrous sodium sulfate, filtered and concentrated under reduced pressure. The residue was purified by flash column chromatography on silica gel (15% ethyl acetate in hexane) to afford 42.4 (0.3 g, 46%). MS(ES): m/z 209.3 [M+H]⁺.

Synthesis of compound 42.5. To a solution of 42.4 (0.3 g, 1.44 mmol, 1.0 equiv) and triethylamine (0.364 g, 3.60 mmol, 2.5 equiv) in DCM (10 mL) at 0° C. was added methanesulfonyl chloride (0.197 g, 1.73 mmol, 1.2 equiv). The reaction mixture was stirred at room temperature for 2 h. It was transferred into ice-water, neutralized with sodium bicarbonate solution and extracted with DCM. The combined organic layers were washed with brine and dried over anhydrous sodium sulfate, filtered and concentrated under reduced pressure to afford 42.5 (0.225 g crude, 54.55%). It was used in the next step without purification.

Synthesis of compound 42.6. To a solution of 42.5 (0.225 g, 0.785 mmol, 1.0 equiv) in dimethyl sulfoxide (8 mL) was added sodium azide (0.153 g, 2.36 mmol, 3.0 equiv) at room temperature. The reaction mixture was stirred at 100° C. for 2 h. It was transferred into ice-water and extracted with ethyl acetate. The combined organic layers were washed with brine and dried over anhydrous sodium sulfate, filtered and concentrated under reduced pressure to afford 42.6 (0.162 g crude, 88.36%). MS(ES): m/z 234.5 [M+H]⁺. It was used in the next step without purification.

Synthesis of compound 42.7. To a solution of compound 42.6 (0.162 g, 6.94 mmol, 1.0 equiv) in THF:water (8 mL: 2 mL) was added triphenylphosphine (0.546 g, 2.08 mmol, 3.0 equiv) at room temperature. The reaction mixture was stirred at 60° C. for 2 h. It was transferred into ice-water and extracted with ethyl acetate. The combined organic layers were washed with brine and dried over anhydrous sodium sulfate, filtered and concentrated under reduced pressure to afford the crude. The residue was purified by flash column chromatography on silica gel (20% ethyl acetate in hexane) to afford 42.7 (0.105 g, 73%). MS(ES): m/z 208.3 [M+H]⁺.

Synthesis of compound 42.8. Compound 42.8 was prepared from compound 42.7 and 1.8 following the procedure described in the synthesis of compound 1.9. The product was purified by flash column chromatography on silica gel (15% ethyl acetate in hexane). MS(ES): m/z 604.5 [M+H]⁺.

Synthesis of compound 42.9. Compound 42.9 was prepared from compound 42.8 following the procedure described in the synthesis of compound 1.10. The product was used without purification. MS(ES): m/z 576.4 [M+H]⁺.

Synthesis of compound 42.10. Compound 42.10 was prepared from compound 42.9 following the procedure described in the synthesis of compound 1.11. The product was purified by flash column chromatography on silica gel (2.0% methanol in DCM). MS(ES): m/z 573.4 [M+H]⁺.

Synthesis of compound 42.11. Compound 42.11 was prepared from compound 42.10 following the procedure described in the synthesis of compound 1.12. The product was purified by flash column chromatography on silica gel (2.1% methanol in DCM. MS(ES): m/z 587.2 [M+H]⁺.

Synthesis of compound 42.12 Compound 42.12 was prepared from compound 42.11 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (2.6% methanol in DCM). MS(ES): m/z 589.3 [M+H]⁺.

Synthesis of compound I-42. Compound I-42 was prepared from compound 42.12 following the procedure described in the synthesis of compound 1.14. The product was purified by flash column chromatography on silica gel (Combiflash®, 3.8% methanol in DCM). MS(ES): m/z 499.06 [M+H]⁺, ¹H NMR (DMSO-d₆, 400 MHz): δ 8.40 (s, 1H), 8.15 (s, 1H), 8.06 (s, 1H), 7.78-7.76 (d, J=8 Hz, 2H), 7.41-7.38 (m, 1H), 7.25-7.24 (d, J=8 Hz, 1H), 7.00-6.98 (d, J=8 Hz, 1H), 4.11 (s, 3H), 3.92-3.88 (m, 1H), 3.65 (s, 3H), 3.60-3.57 (m, 1H), 3.35 (s, 3H), 3.16 (s, 1H), 3.10-3.06 (m, 1H), 2.84-2.81 (m, 1H), 2.14-2.07 (m, 1H), 1.18-1.14 (m, 2H) 0.71-0.69 (m, 1H).

### Example I-43: methyl ((1R,3R)-3-(8-(6-methoxypyrazin-2-yl)-3-methyl-9-(1-methyl-1H-indazol-5-yl)-2-oxo-2,3-dihydro-1H-imidazo[4,5-d]pyrrolo[1,2-b]pyridazin-1-yl)cyclopentyl)carbamate

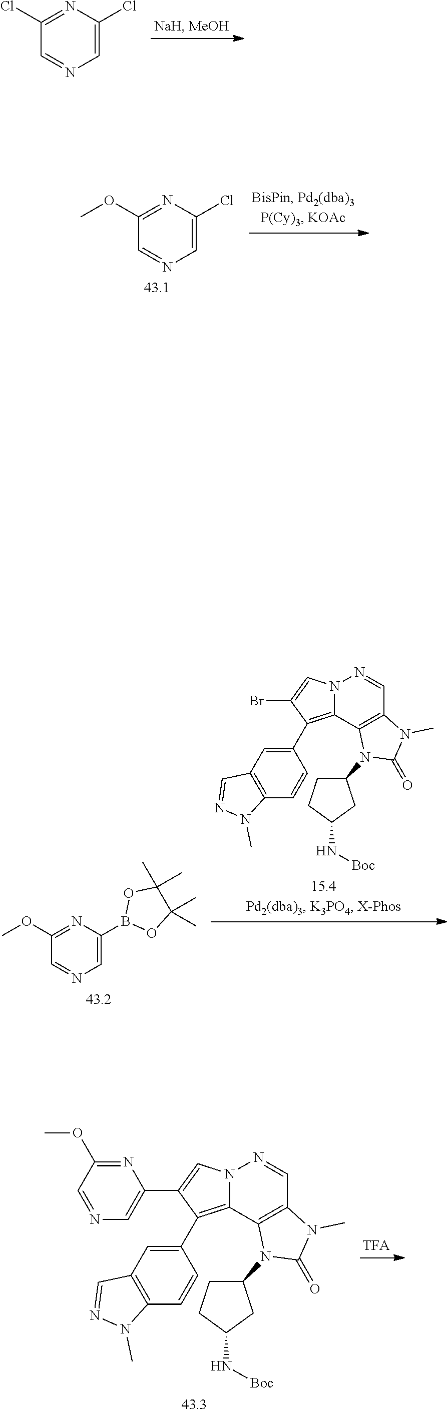

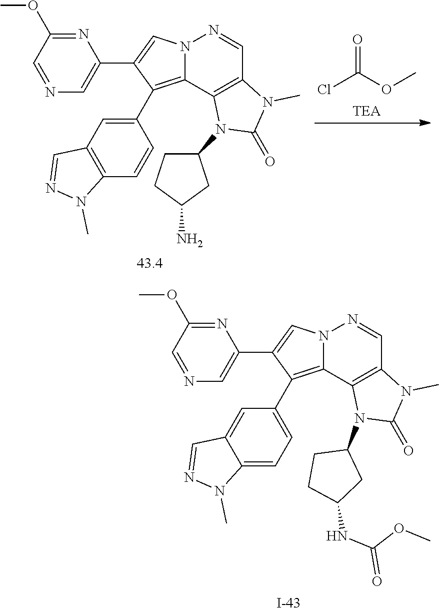

Synthesis of compound 43.1. To a solution of anhydrous MeOH (0.217 g, 6.80 mmol, 1.0 equiv) in THF was added in small portions sodium hydride (60% dispersion in mineral oil) (0.022 g, 13.60 mmol, 1.2 equiv) at 0° C. and stirred for 30 mi. To the mixture was added a solution of 2,6-dichloropyrazine (1 g, 6.80 mmol, 1.0 equiv) in THF (20 mL) and stirred at room temperature for 16 h. It was quenched by ammonium chloride solution and extracted with ethyl acetate. The combined organic layers were washed with brine, dried over anhydrous sodium sulfate, filtered and concentrated under reduced pressure. The residue was purified by flash column chromatography on silica gel (30% ethyl acetate in hexane) to afford 43.1 (0.500 g, 51.5%) H-NMR (CDCl₃) δ 8.16-8.15 (s, 2H) 4.00 (s, 3H).

Synthesis of compound 43.2. A mixture of 43.1 (0.500 g, 3.46 mmol, 1.0 equiv), bis(pinacolato)diboron (0.966 g, 3.80 mmol, 1.1 equiv), tris(dibenzylideneacetone)dipalladium(0) (0.095 g, 0.069 mmol, 0.02 equiv), tricyclohexylphosphine (0.116 g, 0.34 mmol, 0.1 equiv) and potassium acetate (0.509 g, 5.19 mmol, 1.5 equiv) in 1,2-dimethoxyethane (10 mL) was stirred at 150° C. for 3 h. The reaction mixture was cooled to room temperature and filtered through a pad of Celite®, and rinsed with diethyl ether. The filtrate was transferred into water and extracted with diethyl ether. The combined organic layers were washed with brine, dried over anhydrous sodium sulfate, filtered and concentrated under reduced pressure. The residue was used in the next step without purified by column chromatography to afford 43.2 (0.300 g, 37%). MS(ES): m/z 237 [M+H]⁺.

Synthesis of compound 43.3. A mixture of 15.4 (0.150 g, 0.25 mmol, 1.0 equiv), 43.2 (0.122 g, 0.51 mmol, 2.0 equiv), and tripotassium phosphate (0.164 g, 0.77 mmol, 3.0 equiv) in 1,4-dioxane (1.7 mL) and water (0.3 mL) was degassed by bubbling through a stream of argon for 10 min. Tris(dibenzylideneacetone)dipalladium(0) (0.023 g, 0.02 mmol, 0.1 equiv) and 2-dicyclohexylphosphino-2′,4′,6′-triisopropyl-1,1′-biphenyl (0.024 g, 0.05 mmol, 0.2 equiv) were added and degassed for 5 min. The reaction mixture was stirred at 125° C. for 2 h. It was cooled to room temperature, filtered through a pad of Celite® and rinsed with ethyl acetate. The filtrate was transferred into water and extracted with ethyl acetate. The combined organic layers were washed with brine, dried over anhydrous sodium sulfate, filtered and concentrated under reduced pressure. The residue was purified by flash column chromatography on silica gel (50% ethyl acetate in hexane) to afford 43.3 (0.100 g, 63.5%). MS(ES): m/z 610.6 [M+H]⁺.

Synthesis of compound 43.4. Compound 43.4 was prepared from compound 43.3 following the procedure described in the synthesis of compound 1.14. The product was triturated with pentane and dried under reduced pressure. It was used in the next step without purification. MS(ES): m/z 510.5 [M+H]⁺.

Synthesis of I-43. Compound I-43 was prepared from compound 43.4 following the procedure described in the synthesis of compound I-1. The product was purified by flash column chromatography on silica gel (Combiflash®, 0-2.6% methanol in DCM). MS(ES): m/z 568.6 [M+H]⁺, ¹H NMR (DMSO-d₆, 400 MHz): δ 8.55-8.53 (m, 2H), 8.05-8.04 (m, 1H), 7.99-7.97 (m, 2H), 7.88 (s, 1H), 7.77-7.75 (m, 2H), 7.49-7.47 (d, J=8.4 Hz, 1H) 6.73-6.71 (m, 0.6H), 6.35-6.34 (m, 0.4H), 4.14-4.12 (m, 3H), 3.88-3.86 (m, 1H), 3.68 (m, 1H), 3.54 (s, 3H), 3.50 (s, 3H), 3.37 (s, 3H), 2.00-1.97 (m, 1H), 1.82-1.75 (m, 1H), 1.51-1.47 (m, 1H), 1.36-1.34 (m, 1H), 1.18-1.12 (m, 1H).

### Example I-44: 1-((3S,5R)-5-(methoxymethyl)tetrahydrofuran-3-yl)-3-methyl-9-(1-methyl-1H-indazol-5-yl)-8-(1-methyl-1H-pyrazol-4-yl)-1,3-dihydro-2H-imidazo[4,5-d]pyrrolo[1,2-b]pyridazin-2-one

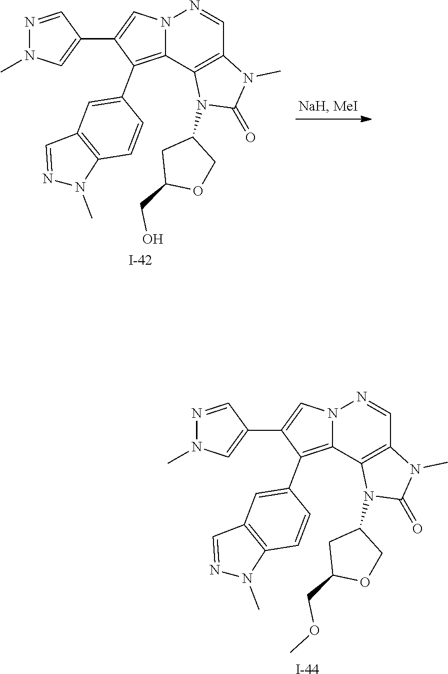

Synthesis of I-44. To a solution of I-42 (0.02 g, 0.04 mmol, 1.0 equiv) in DMF (5 mL) at 0° C. was added sodium hydride (0.003 g, 0.08 mmol, 2.0 equiv) followed by dropwise addition of methyl iodide (0.011 g, 0.08 mmol, 2.0 equiv). The reaction mixture was stirred for 10 min. It was transferred into ice-water and extracted with ethyl acetate. The combined organic layers were washed with brine and dried over anhydrous sodium sulfate, filtered and concentrated under reduced pressure. The residue was purified by flash column chromatography on silica gel (3.1% methanol in DCM) to afford I-44 (0.010 g, 49%). MS(ES): m/z 513.7 [M+H]⁺, ¹H NMR (DMSO-d₆, 400 MHz): δ 8.41 (s, 1H), 8.16-8.15 (d, J=4 Hz, 1H), 8.09-8.08 (d, J=4 Hz, 1H), 7.79-7.77 (m, 2H), 7.42-7.40 (m, 1H), 7.25 (s, 1H), 6.97-6.95 (d, J=8 Hz, 1H), 4.13 (s, 3H), 4.01 (s, 1H), 3.94 (s, 1H), 3.69-3.68 (m, 2H), 3.61 (s, 3H), 3.57-3.37 (m, 1H), 3.30 (s, 3H), 3.29-3.27 (d, J=8 Hz, 1H), 3.06 (s, 1H), 3.01 (s, 1H), 2.41-2.11 (m, 3H).

### Example I-45: (1R,2R)-2-fluoro-N-((1s,4S)-1-methyl-4-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclohexyl)cyclopropane-1-carboxamide

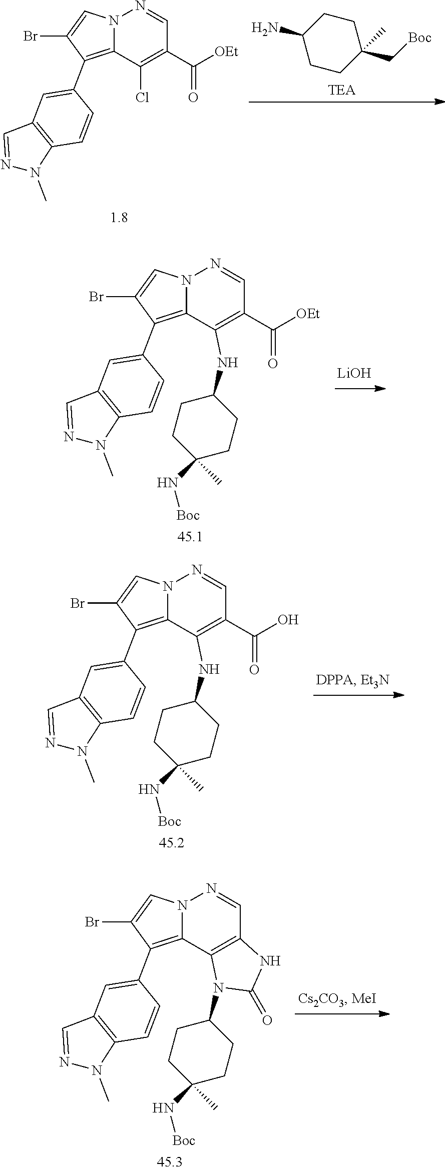

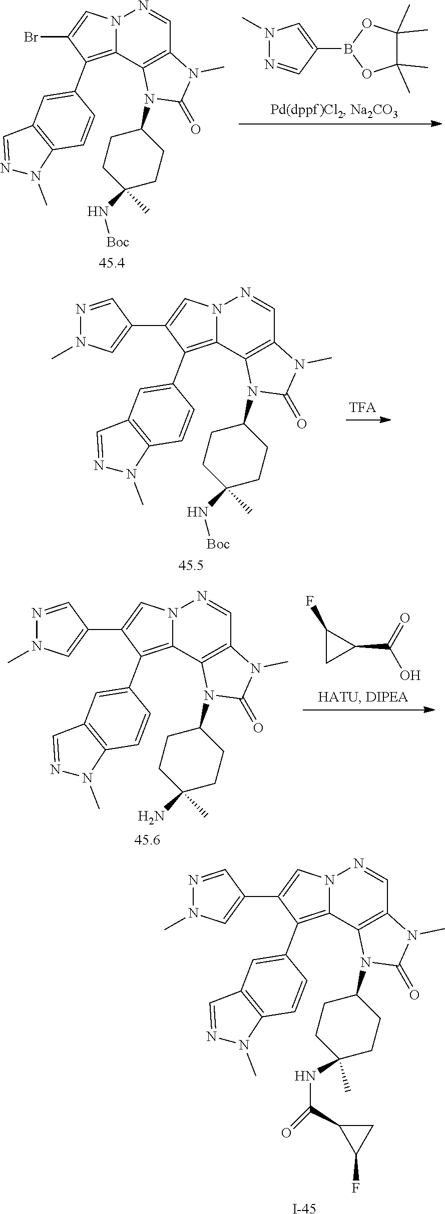

Synthesis of compound 45.1. Compound 45.1 was prepared from compound 1.8 and tert-butyl ((1s,4s)-4-amino-1-methylcyclohexyl)carbamate following the procedure described in the synthesis of compound 1.9. The product was purified by flash column chromatography on silica gel (Combiflash®, 53% ethyl acetate in hexane). MS(ES): m/z 626.2 and 628.2 [M+H]⁺.

Synthesis of compound 45.2. Compound 45.2 was prepared from compound 45.1 following the procedure described in the synthesis of compound 1.10. The product was triturated with pentane and used in the next step without purification. MS(ES): m/z 598.3 and 600.3 [M+H]⁺.

Synthesis of compound 45.3. Compound 45.3 was prepared from compound 45.2 following the procedure described in the synthesis of compound 1.11. The product was purified by flash column chromatography on silica gel (Combiflash®, 1.8% methanol in DCM). MS(ES): m/z 595.4 and 597.4 [M+H]⁺.

Synthesis of compound 45.4. Compound 45.4 was prepared from compound 45.3 following the procedure described in the synthesis of compound 1.12. The product was purified by flash column chromatography on silica gel (1.2% methanol in DCM). MS(ES): m/z 609.2 and 611.2 [M+H]⁺.

Synthesis of compound 45.5. Compound 45.5 was prepared from compound 45.4 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (Combiflash®, 0-2.8% methanol in DCM). MS(ES): m/z 610.2 [M+H]⁺.

Synthesis of compound 45.6. Compound 45.6 was prepared from compound 45.5 following the procedure described in the synthesis of compound 1.14. The product was triturated with pentane and dried under reduced pressure. It was used in the next step without purification. MS(ES): m/z 510.1 [M+H]⁺.

Synthesis of I-45. To a solution of 45.6 (0.085 g, 0.166 mmol, 1.0 equiv), HATU (0.095 g, 0.25 mmol, 1.5 equiv), N,N-diisopropylethylamine (0.043 g, 0.33 mmol, 2.0 equiv) and (1R,2R)-2-fluorocyclopropane-1-carboxylic acid (0.052 g, 0.50 mmol, 3.0 equiv) in DMF (5 mL) were added and stirred at room temperature for 1 h. It was quenched with water and extracted with ethyl acetate. The combined organic layers were washed with brine, dried over anhydrous sodium sulfate, filtered and concentrated under reduced pressure. The residue was purified by flash column chromatography on silica gel (Combiflash®, 0-2.4% methanol in DCM) to afford I-45 (0.045 g, 45%). MS(ES): m/z 596.2 [M+H]⁺, ¹H NMR (DMSO-d₆, 400 MHz): δ 8.38 (d, J=3.2 Hz, 1H), 8.14-8.03 (m, 2H), 7.83-7.72 (m, 2H), 7.42-7.37 (m, 1H), 7.28 (s, 1H), 7.22-7.13 (m, 1H), 6.89 (s, 1H), 4.76 (s, 0.4H), 4.60 (s, 0.4H), 4.13 (s, 3H), 3.64 (s, 3H), 3.33 (s, 3H), 3.17-3.11 (m, 1H), 2.36-2.22 (m, 1H), 1.95-1.66 (m, 2H), 1.58-1.55 (m, 2H), 1.40-1.33 (m, 3H), 0.98-0.84 (m, 3H), 0.66 (s, 3H).

### Example I-46: methyl ((1R,3R)-3-(3-methyl-9-(1-methyl-1H-indazol-5-yl)-8-(2-methylpyrimidin-4-yl)-2-oxo-2,3-dihydro-1H-imidazo[4,5-d]pyrrolo[1,2-b]pyridazin-1-yl)cyclopentyl)carbamate

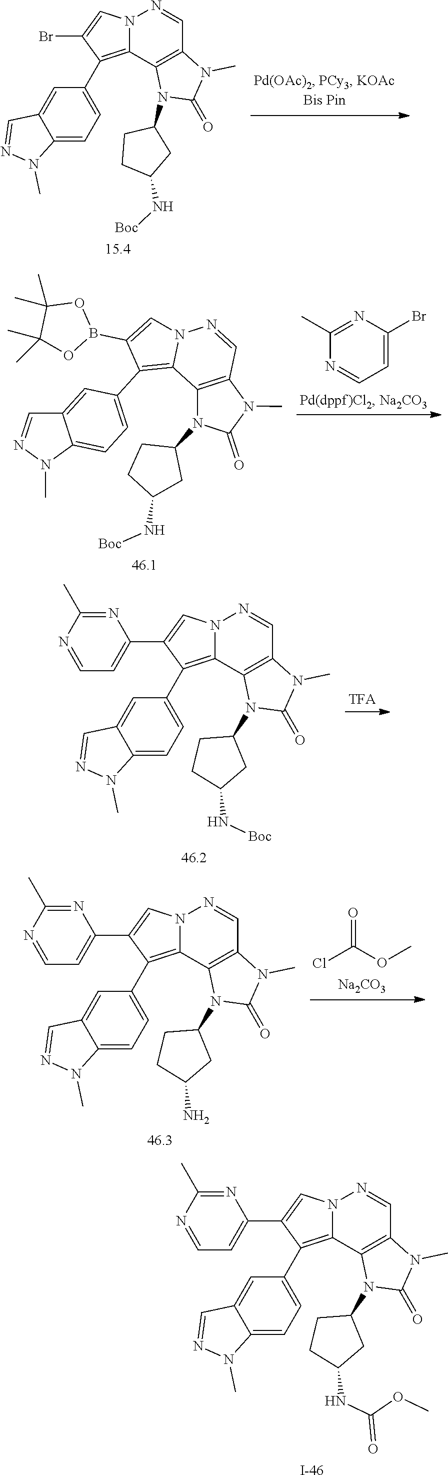

Synthesis of compound 46.1. To a solution of 15.4 (0.200 g, 0.34 mmol, 1.0 equiv) was added bis(pinacolato) diborane (0.262 g, 1.03 mmol, 3.0 equiv), palladium(II) acetate (0.077 g, 0.03 mmol, 0.1 equiv), tricyclohexylphosphine (0.019 g, 0.06 mmol, 0.2 equiv) and potassium acetate (0.168 g, 1.72 mmol, 5.0 equiv) in dioxane (10 mL). The reaction mixture was stirred at 110° C. for 1 h. It was cooled to room temperature, filtered through a pad of Celite® and rinsed with ethyl acetate. The filtrate was concentrated under reduced pressure to afford 46.1 (0.230 g, 65%). MS(ES): m/z 628.5 [M+H]⁺. It was used in the next step without purification.

Synthesis of compound 46.2. A mixture of 46.1 (0.230 g, 0.36 mmol, 1.0 equiv), 4-bromo-2-methylpyrimidine (0.126 g, 0.73 mmol, 2.0 equiv) and sodium carbonate (0.114 g, 1.08 mmol, 3.0 equiv) in 1,4-dioxane (5 mL) and water (0.5 mL) was degassed by bubbling through a stream of argon for 10 min. (1,1′-Bis(diphenylphosphino)ferrocene) palladium (II) dichloride (0.026 g, 0.03 mmol, 0.1 equiv) was added and degassed for 5 min. The reaction mixture was stirred at 110° C. for 2 h. It was cooled to room temperature, filtered through a pad of Celite® and rinsed with ethyl acetate. The filtrate was transferred into water and extracted with ethyl acetate. The combined organic layers were washed with brine, dried over anhydrous sodium sulfate, filtered and concentrated under reduced pressure. The residue was purified by flash column chromatography on silica gel (5% methanol in DCM) to afford 46.2 (0.120 g, 55%). MS(ES): m/z 594.6 [M+H]⁺.

Synthesis of compound 46.3. Compound 46.3 was prepared from compound 46.2 following the procedure described in the synthesis of compound 1.14. The product was triturated with pentane and dried under reduced pressure. It was used in the next step without purification. MS(ES): m/z 494.5 [M+H]⁺.

Synthesis of I-46. Compound I-46 was prepared from compound 46.3 following the procedure described in the synthesis of compound I-1. The product was purified by flash column chromatography on silica gel (Combiflash®, 0-2.6% methanol in DCM). MS(ES): m/z 552.3 [M+H]⁺, ¹H-NMR (400 MHz, DMSO-d₆): δ 8.50 (s, 2H), 8.31-8.27 (m, 1H), 8.06 (s, 1H), 7.81-7.78 (m, 2H), 7.46 (d, J=8.4 Hz, 1H), 6.75-6.73 (m, 0.5H), 6.59 (d, J=5.2 Hz, 0.7H), 6.47-6.48 (m, 0.5H), 6.35 (bs, 0.3), 4.14 (s, 3H), 3.85 (m, 1H), 3.53 (s, 1H), 3.48 (bs, 1H), 3.47 (s, 3H), 3.36 (s, 3H), 3.34 (s, 3H), 1.98-1.96 (m, 1H), 1.81-1.79 (m, 1H), 1.50-1.47 (m, 1H), 1.23-1.13 (m, 1H), 1.11-1.04 (m, 1H).

### Example I-47: methyl ((1r,3r)-3-(8-(3,6-dihydro-2H-pyran-4-yl)-3-methyl-9-(1-methyl-1H-indazol-5-yl)-2-oxo-2,3-dihydro-1H-imidazo[4,5-d]pyrrolo[1,2-b]pyridazin-1-yl)cyclobutyl)carbamate

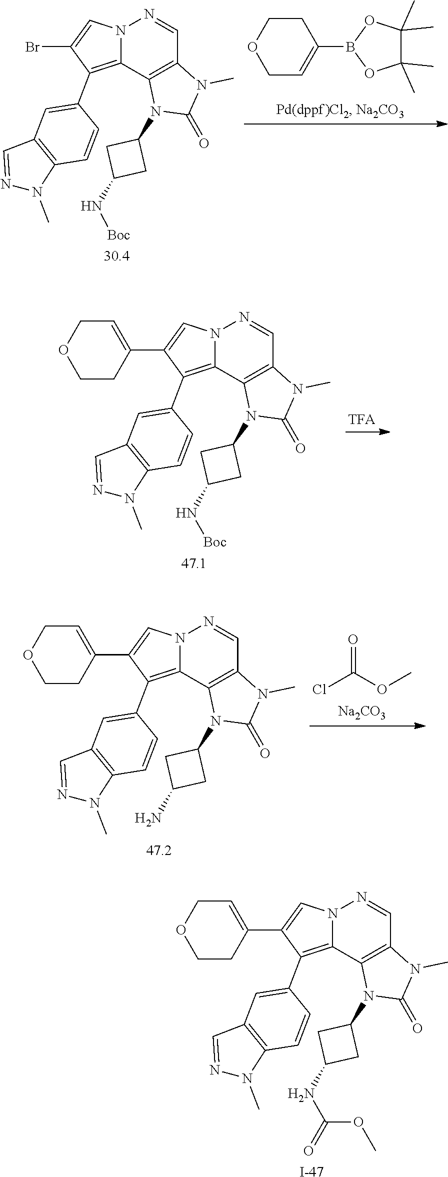

Synthesis of compound 47.1. Compound 47.1 was prepared from compound 30.4 and 2-(3,6-dihydro-2H-pyran-4-yl)-4,4,5,5-tetramethyl-1,3,2-dioxaborolane following the procedure described in the synthesis of compound 1.13. The product was purified by flash column chromatography on silica gel (Combiflash®, 3.1% methanol in DCM). MS(ES): m/z 570.5 [M+H]⁺.

Synthesis of compound 47.2. Compound 47.2 was prepared from compound 47.1 following the procedure described in the synthesis of compound 1.14. The product is used in the next step without purification. MS(ES): m/z 470.4 [M+H]⁺.

Synthesis of I-47. Compound I-47 was prepared from compound 47.2 following the procedure described in the synthesis of compound I-1. The product was purified by flash column chromatography on silica gel (Combiflash®, 2.6% methanol in DCM). MS(ES): m/z 528.5 [M+H]⁺, ¹H NMR (DMSO-d₆, 400 MHz): δ 8.42 (s, 1H), 8.03 (s, 1H), 7.93 (s, 1H), 7.76 (d, J=8.8 Hz, 1H), 7.66 (s, 1H), 7.38 (d, J=8.4 Hz, 1H), 6.32 (s, 1H), 5.39 (s, 1H), 4.12 (s, 3H), 3.92-3.92 (m, 2H), 3.82-3.80 (m, 2H), 3.63-3.60 (m, 2H), 3.47 (s, 2H), 3.38 (s, 3H), 2.72 (s, 1H), 2.56-2.52 (m, 1H), 2.11 (s, 2H), 1.67 (s, 1H), 1.24-1.08 (m, 2H).

### Example I-48: methyl ((1R,3R)-3-(3-methyl-9-(1-methyl-1H-indazol-5-yl)-2-oxo-8-(1,3,4-thiadiazol-2-yl)-2,3-dihydro-1H-imidazo[4,5-d]pyrrolo[1,2-b]pyridazin-1-yl)cyclopentyl)carbamate

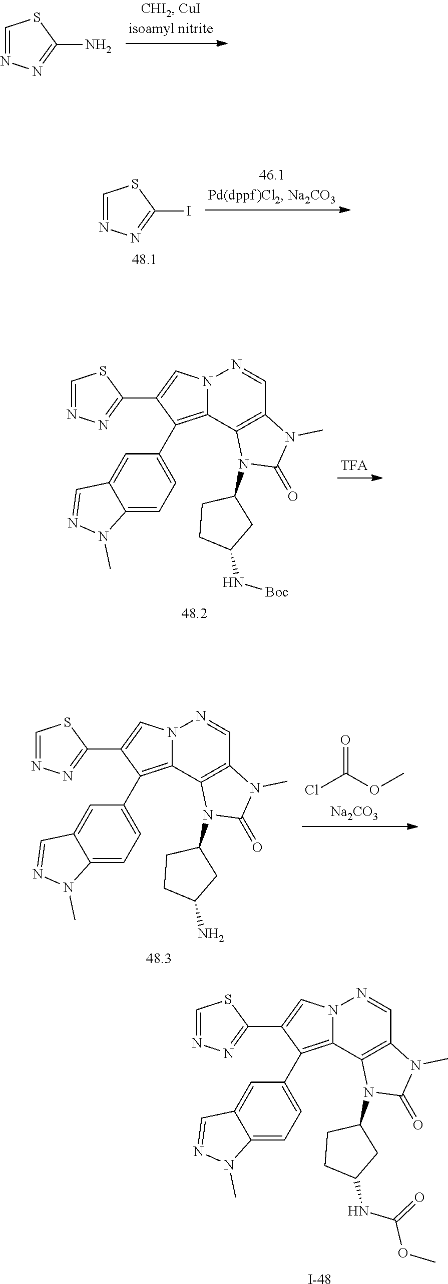

Synthesis of compound 48.1. A mixture of 1,3,4-thiadiazol-2-amine (8.0 g, 79.11 mmol, 1.0 equiv), diiodomethane (95 g, 355.97 mmol, 4.5 equiv), copper iodide (15 g, 79.11 mmol, 1.0 equiv) and isoamyl nitrite (23.13 g, 197.76 mmol, 2.5 equiv) in THF (80 mL) was stirred at 90° C. for 6 h. It was cooled to room temperature, filtered through a pad of Celite® and rinsed with ethyl acetate. The filtrate was concentrated under reduced pressure. The residue was purified by flash column chromatography on silica gel (15% ethyl acetate in hexane) to afford 48.1 (2.0 g, 12%) ¹H-NMR (400 MHz, CDCl₃): δ 9.13 (s, 1H).

Synthesis of compound 48.2. Compound 48.2 was prepared from compound 48.1 and 46.1 following the procedure described in the synthesis of compound 46.2. The product was purified by flash column chromatography on silica gel (2.1% methanol in DCM). MS(ES): m/z 586.4 [M+H]⁺.

Synthesis of compound 48.3. Compound 48.3 was prepared from compound 48.2 following the procedure described in the synthesis of compound 1.14. The product was triturated with pentane and dried under reduced pressure and used in the next step without purification. MS(ES): m/z 486.5 [M+H]⁺.

Synthesis of I-48. Compound I-48 was prepared from compound 48.3 following the procedure described in the synthesis of compound I-1. The product was purified by flash column chromatography on silica gel (Combiflash®, 2.3% methanol in DCM). MS(ES): m/z 544.4 [M+H]⁺, 1H-NMR (400 MHz, DMSO-d6): δ₁H-NMR (400 MHz, DMSO-d₆): δ 9.23-9.21 (m, 1H), 8.59-8.57 (m, 2H), 8.11-8.10 (m, 1H), 7.87-7.81 (m, 2H), 7.45-7.42 (m, 1H), 6.73-6.41 (m, 1H), 4.16-4.14 (m, 3H), 3.96-3.79 (m, 2H), 3.49-3.44 (m, 6H), 2.03-2.02 (bs, 1H), 1.84-1.72 (m, 2H), 1.53-1.37 (m, 1H), 1.15-1.10 (m, 1H), 0.81-0.36 (m, 1H).

### Example I-49: methyl ((1R,3R)-3-(8-(2,3-dihydropyrazolo[5,1-b]oxazol-7-yl)-9-(4-(2-hydroxypropan-2-yl)phenyl)-3-methyl-2-oxo-2,3-dihydro-1H-imidazo[4,5-d]pyrrolo[1,2-b]pyridazin-1-yl)cyclopentyl)carbamate

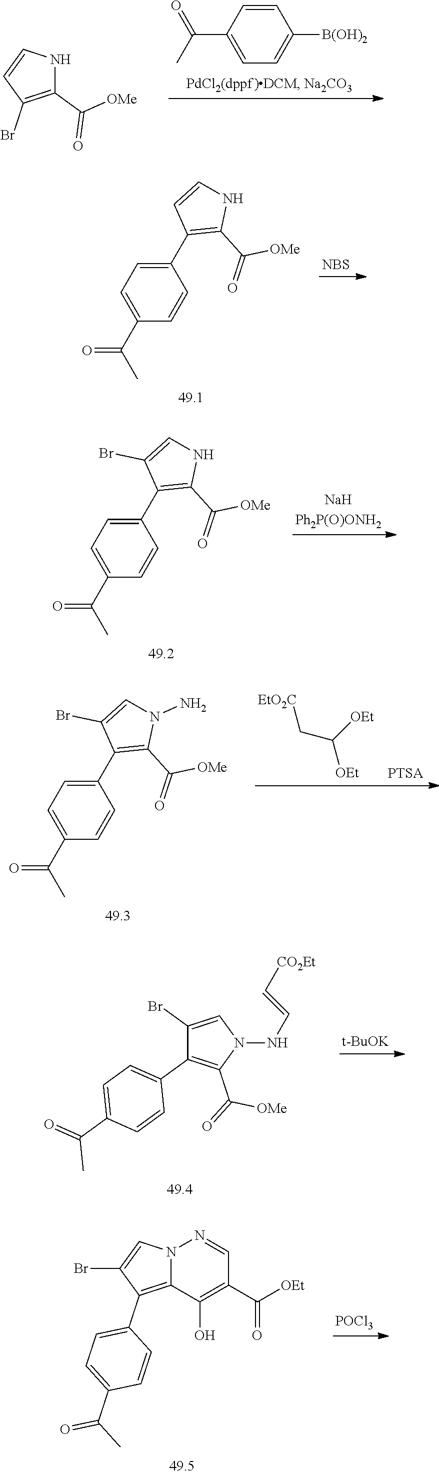

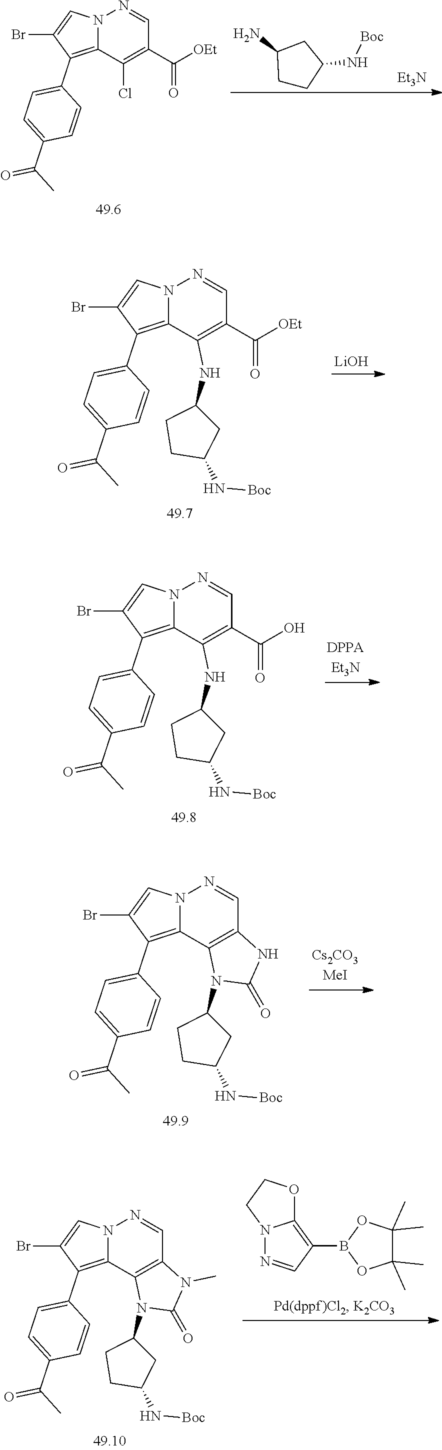

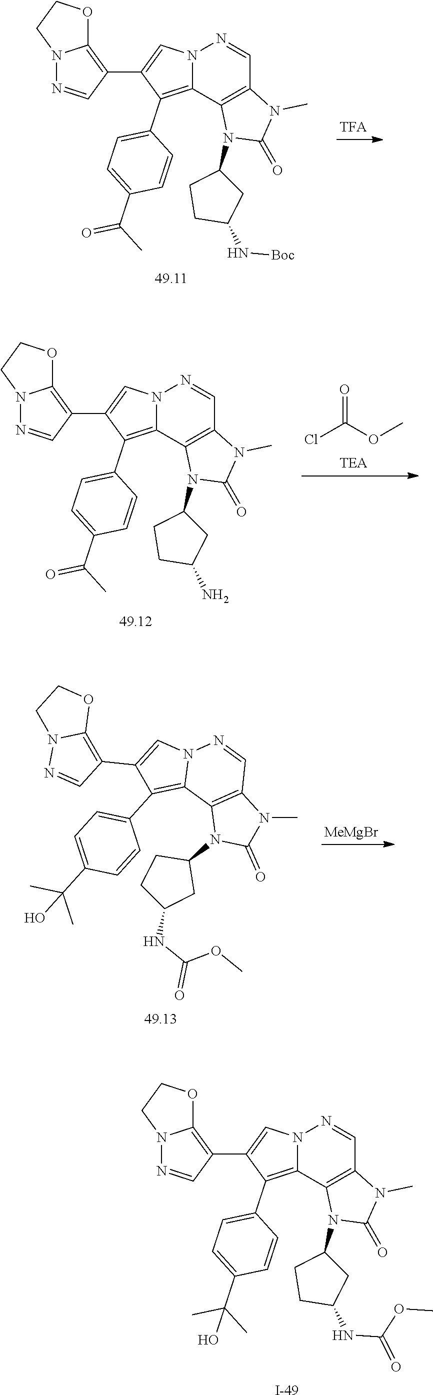

Synthesis of compound 49.1. Compound 49.1 was prepared from compound methyl 3-bromo-1H-pyrrole-2-carboxylate and (4-acetylphenyl)boronic acid following the procedure described in the synthesis of compound 1.2. The product was purified by flash column chromatography on silica gel (16% ethyl acetate in n-hexane). MS (ES): m/z 244.26 [M+H]⁺.

Synthesis of compound 49.2. Compound 49.2 was prepared from compound 49.1 following the procedure described in the synthesis of compound 1.4. The product was purified by flash column chromatography on silica gel (18% ethyl acetate in n-hexane). MS (ES): m/z 323.3 [M+H]⁺ and m/z 324.3 [M+2]⁺.

Synthesis of compound 49.3. Compound 49.3 was prepared from compound 49.2 following the procedure described in the synthesis of compound 1.5. The product was purified by flash column chromatography on silica gel (18% ethyl acetate in n-hexane). MS (ES): m/z 338.17 and 340.17 [M+H]⁺.

Synthesis of compound 49.4. Compound 49.4 was prepared from compound 49.3 following the procedure described in the synthesis of compound 1.6. The product was purified by flash column chromatography on silica gel (12% ethyl acetate in n-hexane). MS(ES): m/z 436.27 and 437.27 [M+H]⁺.

Synthesis of compound 49.5. Compound 49.5 was prepared from compound 49.4 following the procedure described in the synthesis of compound 1.7. The product was purified by flash column chromatography on silica gel (30% ethyl acetate in n-hexane). MS(ES): m/z 403.36 and 405.36 [M+H]⁺.

Synthesis of compound 49.6. Compound 49.6 was prepared from compound 49.5 following the procedure described in the synthesis of compound 1.8. The product was purified by flash column chromatography on silica gel (30% ethyl acetate in n-hexane). MS(ES): m/z 422.68 [M+H]⁺.

Synthesis of compound 49.7. Compound 49.7 was prepared from compound 49.6 and tert-butyl ((1R,3R)-3-aminocyclopentyl)carbamate following the procedure described in the synthesis of compound 1.9. The product was purified by flash column chromatography on silica gel (15% ethyl acetate in n-hexane). MS(ES): m/z 586.5 and 588.5 [M+H]⁺.

Synthesis of compound 49.8. Compound 49.8 was prepared from compound 49.7 following the procedure described in the synthesis of compound 1.10. The product was used in the next step without purification. MS (ES): m/z 558.45 and 560.45 [M+H]⁺.

Synthesis of compound 49.9. Compound 49.9 was prepared from compound 49.8 following the procedure described in the synthesis of compound 1.11. The product was purified by flash column chromatography on silica gel (2.1% methanol in DCM). MS(ES): m/z 555.4 and 557.4 [M+H]⁺.

Synthesis of compound 49.10. Compound 49.10 was prepared from compound 49.9 following the procedure described in the synthesis of compound 1.12. The product was purified by flash column chromatography on silica gel (2.5% methanol in DCM. MS(ES): m/z 569.4 [M+H]⁺.

Synthesis of compound 49.11. Compound 49.11 was prepared from compound 49.10 and 7-(4,4,5,5-tetramethyl-1,3,2-dioxaborolan-2-yl)-2,3-dihydropyrazolo[5,1-b]oxazole following the procedure described in the synthesis of compound 1.13. The product was purified by flash column chromatography on silica gel (16% ethyl acetate in n-hexane). MS (ES): m/z 598.6

Synthesis of compound 49.12. Compound 49.12 was prepared from compound 49.11 following the procedure described in the synthesis of compound 1.14. The product was used in the next step without purification. MS (ES): m/z 498.5 [M+H]⁺.

Synthesis of compound 49.13. Compound 49.13 was prepared from compound 49.12 following the procedure described in the synthesis of compound I-1. The product was purified by flash column chromatography on silica gel (3.2% methanol in DCM). MS(ES): m/z 556.5 [M+H]⁺.

Synthesis of compound I-49. To a solution of 49.13 (0.080 g, 0.14 mmol, 1.0 equiv) in THF (5 mL) was added dropwise methyl magnesium bromide (3.0M in THF) (0.07 mL, 0.21 mmol, 1.5 equiv) at −78° C. The reaction mixture was stirred at same temperature for 10 min. It was transferred to ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 4.5% methanol in DCM) to afford I-49 (0.010 g, 12%). MS (ES): m/z 572.3 [M+H]⁺, ¹H NMR (DMSO-d₆, 400 MHz): δ 8.37 (s, 1H), 7.80 (s, 1H), 7.61 (dd, J=8 Hz, 2H), 7.33 (dd, J=8.4 Hz, 2H), 6.75-6.80 (d, J=6.8 Hz, 1H), 6.36 (s, 1H), 5.10 (t, J=4.8 Hz, 2H), 4.25-4.21 (m, J=8 Hz, 1H), 4.05 (m, 1H), 3.85 (s, 1H), 3.49 (s, 3H), 3.39 (t, J=Hz, 3H), 3.37-3.24 (m, 6H), 2.04 (m, J=3.2 Hz, 1H), 1.88 (m, 1H), 1.785 (m, 1H), 1.539-1.484 (d, J=9.2 Hz, 5H).

### Example I-50: methyl ((1R,3R)-3-(9-(4-(2-hydroxypropan-2-yl)phenyl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

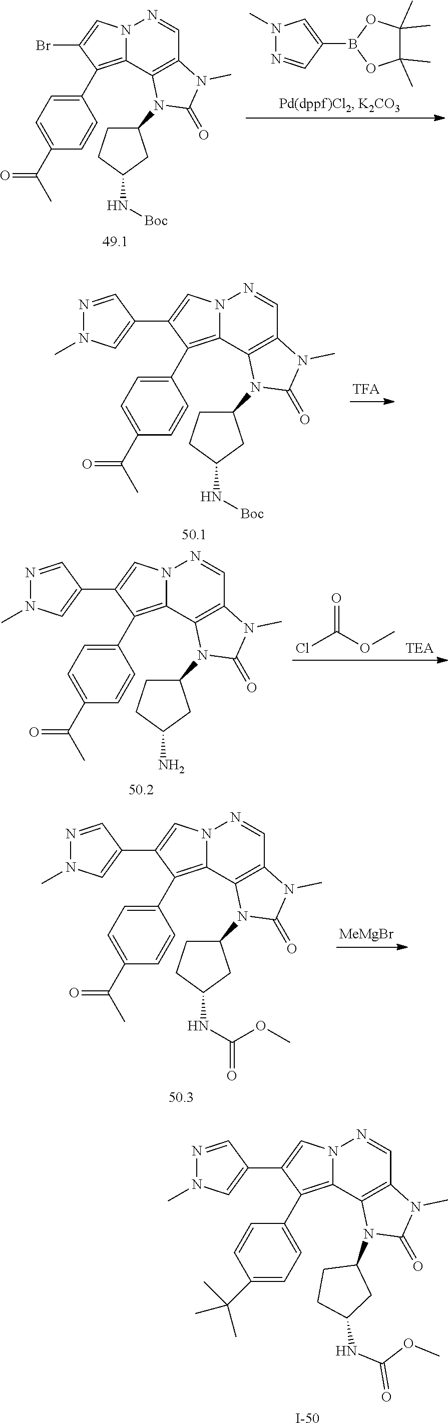

Synthesis of compound 50.1. Compound 50.1 was prepared from compound 49.10 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (46% ethyl acetate in hexane). MS(ES): m/z 570.6 [M+H]⁺.

Synthesis of compound 50.2. Compound 50.2 was prepared from compound 50.1 following the procedure described in the synthesis of compound 1.14. The product was used in the next step without purification.

Synthesis of compound 50.3. Compound 50.3 was prepared from compound 50.2 following the procedure described in the synthesis of compound I-1. The product was purified by flash column chromatography on silica gel (3.2% methanol in DCM). MS(ES): m/z 528.2 [M+H]⁺.

Synthesis of compound I-50. To a solution of 50.3 (0.085 g, 0.16 mmol, 1.0 equiv) in THF (5 mL) was added dropwise methyl magnesium chloride (3.0 M in THF, 0.08 mL, 0.24 mmol, 1.5 equiv) at 0° C. The reaction mixture was stirred at same temperature for 10 min. It was transferred to ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 4.5% methanol in DCM) to afford I-50 (0.012 g, 13%). MS(ES): m/z 544.6 [M+H]⁺, ¹H NMR (DMSO-d₆, 400 MHz): δ 8.38 (s, 1H), 8.12 (s, 1H), 7.60 (dd, J=7.6 Hz, 2H), 7.34 (d, J=7.6 Hz, 1H), 7.29-7.26 (m, 2H), 6.98 (s, 1H), 6.81 (d, J=6.0 Hz, 1H), 5.11 (s, 1H), 4.02 (bs, 1H), 3.83 (bs, 1H), 3.69 (s, 3H), 3.48 (s, 3H), 3.34 (s, 3H), 2.03 (bs, 1H), 1.88 (bs, 1H), 1.77 (bs, 1H), 1.52 (d, J=9.2 Hz, 6H), 1.35 (bs, 1H), 1.16-1.14 (m, 1H), 0.65 (bs, 1H).

### Example I-51: methyl ((1R,3R)-3-(9-(4-(2-hydroxypropan-2-yl)phenyl)-3-methyl-8-(2-methyl-2H-1,2,3-triazol-4-yl)-2-oxo-2,3-dihydro-1H-imidazo[4,5-d]pyrrolo[1,2-b]pyridazin-1-yl)cyclopentyl)carbamate

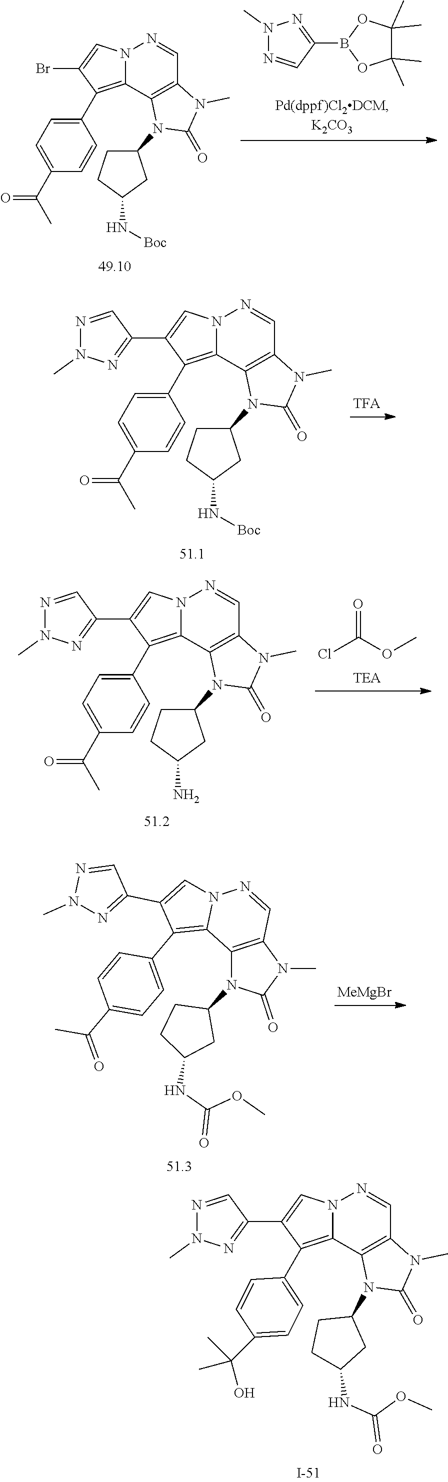

Synthesis of compound 51.1. A mixture of 49.10 (0.4 g, 0.703 mmol, 1.0 equiv), 2-methyl-4-(4,4,5,5-tetramethyl-1,3,2-dioxaborolan-2-yl)-2H-1,2,3-triazole (0.191 g, 0.91 mmol, 1.3 equiv) and potassium carbonate (0.291 g, 2.11 mmol, 3.0 equiv) in 1,4-dioxane (12 mL) and water (2 mL) was degassed by bubbling through a stream of argon for 10 min. [1,1′-Bis(diphenylphosphino)ferrocene]dichloropalladium DCM complex (II) (0.057 g, 0.070 mmol, 0.1 equiv) was added and degassed for 5 min. The reaction mixture was stirred at 100° C. for 1 h. It was transferred to ice-water and extracted with ethyl acetate. The combined organic layers were washed with brine, dried over anhydrous sodium sulfate, filtered and concentrated under reduced pressure. The residue was purified by flash column chromatography on silica gel (46% ethyl acetate in hexane) to afford 51.1 (0.32 g, 80%). MS(ES): m/z 571.6 [M+H]⁺.

Synthesis of compound 51.2. Compound 50.2 was prepared from compound 50.1 following the procedure described in the synthesis of compound 1.14. The product was used in the next step without purification. MS(ES): m/z 471.5 [M+H]⁺.

Synthesis of compound 51.3. Compound 51.3 was prepared from compound 51.2 following the procedure described in the synthesis of compound I-1. The product was purified by flash column chromatography on silica gel (3.2% methanol in DCM). MS(ES): m/z 529.5 [M+H]⁺.

Synthesis of compound I-51. Compound I-51 was prepared from compound 51.3 following the procedure described in the synthesis of compound I-50. The product was purified by flash column chromatography on silica gel (Combiflash®, 4.5% methanol in DCM). MS(ES): m/z 545.6 [M+H]⁺, ¹H NMR (DMSO-d₆, 400 MHz): δ 8.52 (s, 1H), 8.16 (s, 1H), 7.61 (dd, J=7.6 Hz, 2H), 7.35 (dd, J=7.2 Hz, 2H), 6.79 (d, J=6.0 Hz, 1H), 6.55 (s, 1H), 5.11 (s, 1H), 4.15 (s, 3H), 4.09 (s, 1H), 3.86 (d, J=6.8 Hz, 1H), 3.48 (s, 3H), 3.37 (s, 3H), 2.55 (m, 1H), 2.07 (d, J=12.0 Hz, 1H), 1.88 (d, J=8.0 Hz, 1H), 1.78 (t, J=5.6 Hz, 1H), 1.52 (d, J=8.8 Hz, 6H), 1.35 (t, J=13.2 Hz, 1H), 1.23-1.13 (m, 1H).

### Example I-52: methyl ((1R,3R)-3-(3-methyl-8-(1-methyl-1H-pyrazol-4-yl)-2-oxo-9-(thieno[2,3-c]pyridin-2-yl)-2,3-dihydro-1H-imidazo[4,5-d]pyrrolo[1,2-b]pyridazin-1-yl)cyclopentyl)carbamate

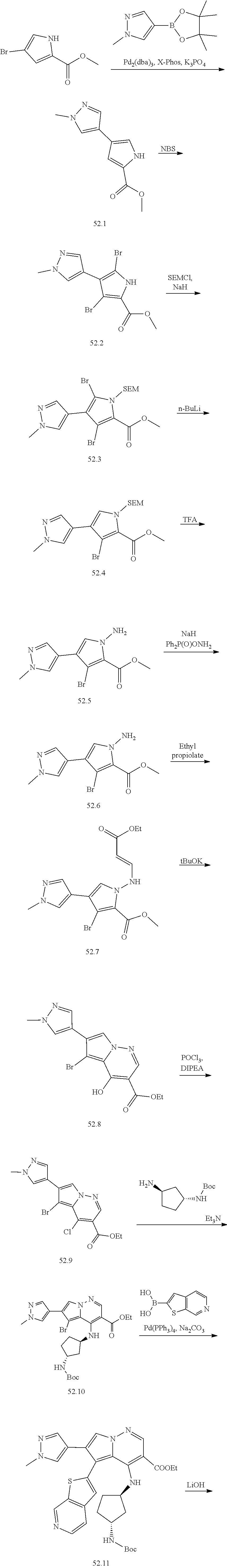

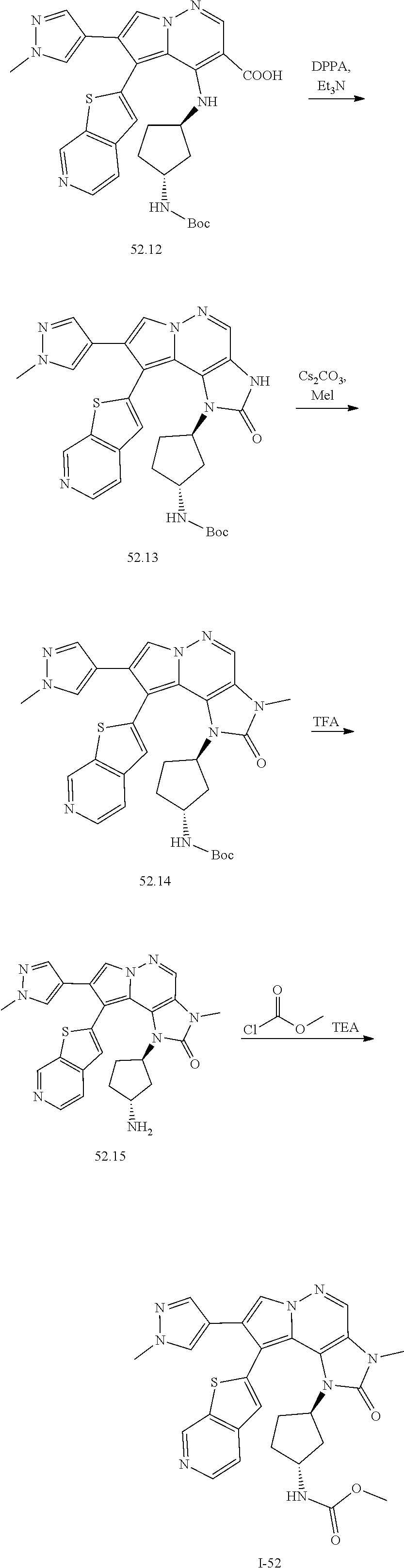

Synthesis of compound 52.1. A mixture of methyl 4-bromo-1H-pyrrole-2-carboxylate (100 g, 480.7 mmol, 1.0 equiv), 1-methyl-4-(4,4,5,5-tetramethyl-1,3,2-dioxaborolan-2-yl)-1H-pyrazole (108 g, 528.8 mmol, 1.1 equiv) and potassium phosphate tribasic anhydrous (141 g, 1442.1 mmol, 3 equiv) in toluene (750 mL) and water (250 mL) was degassed by bubbling through a stream of argon for 10 min. Tris(dibenzylideneacetone)dipalladium (0) (22 g, 24.0 mmol, 0.05 equiv) and [2-dicyclohexylphosphino-2′,4′,6′-triisopropylbiphenyl](23 g, 48.0 mmol, 0.1 equiv) were added and degassed for 5 min. The reaction mixture was stirred at 110° C. for 16 h. It was cooled to room temperature, transferred into water, extracted with ethyl acetate. The combined organic layers were washed with brine, dried over anhydrous sodium sulfate, filtered and concentrated under reduced pressure. The residue was purified by flash column chromatography on silica gel (30% ethyl acetate in hexane) to afford 52.1 (62 g, 62%). MS(ES): m/z 206.1 [M+H]⁺. ¹H NMR (DMSO-d₆, 400 MHz): δ 11.86 (s, 1H), 7.84-7.83 (bs, 1H), 7.63-7.62 (bs, 1H) 7.21-7.20 (bs, 1H), 6.93-6.92 (bs, 1H), 3.83-3.71 (s, 6H).

Synthesis of compound 52.2. To a solution of 52.1 (52 g, 253.1 mmol, 1.0 equiv) in chloroform (1000 mL) was added N-bromosuccinimide (90 g, 507.3 mmol, 2 equiv) in small portions. The reaction mixture was stirred at 85° C. for 16 h. It was cooled to room temperature, quenched with 10% sodium thiosulfate solution, and extracted with chloroform. The combined organic layers were washed with brine, dried over anhydrous sodium sulfate, filtered and concentrated under reduced pressure. The residue was purified by flash column chromatography on silica gel (65% ethyl acetate in hexane) to afford 52.2 (80 g, 87%). MS(ES): m/z 363.9 and 365.9 [M+H]⁺, ¹H NMR (DMSO-d₆, 400 MHz): δ 13.18 (s, 1H), 7.92 (s, 1H), 7.62 (s, 1H) 3.87 (s, 3H), 3.79 (s, 3H).

Synthesis of compound 52.3. To a solution of 52.2 (80 g, 220.3 mmol, 1.0 equiv) in DMF (800 mL) was added 60% sodium hydride (17.6 g, 440.6 mmol, 2 equiv) in small portions at 0° C. and stirred for 30 min. To the suspension was added 2-(trimethylsilyl) ethoxymethyl chloride (55 g, 330.5 mmol, 1.5 equiv). The reaction mixture was stirred at 0° C. for 2 h. It was quenched with ice-water and extracted with ethyl acetate. The combined organic layers were washed with brine, dried over anhydrous sodium sulfate, filtered and concentrated under reduced pressure to afford 52.3 (98 g, 90%). MS(ES): m/z 492.03, 494.05 and 496.06 [M+H]⁺, ¹H NMR (DMSO-d₆, 400 MHz): δ 7.93 (s, 1H), 7.60 (s, 1H), 5.75 (s, 2H) 3.89 (s, 3H), 3.82 (s, 3H), 3.54-3.50 (m, 2H), 0.84-0.80 (m, 2H), −0.41 (s, 9H).

Synthesis of compound 52.4. To a solution of 52.3 (67 g, 136 mmol, 1.0 equiv) in anhydrous THF (1350 mL) was added dropwise n-butyllithium (2.5M in hexane, 43 mL, 0.8 equiv) at −78° C. The reaction mixture was stirred at −78° C. for 1 h. It was cooled to room temperature and quenched with water and extracted with ethyl acetate. The combined organic layers were washed with brine, dried over anhydrous sodium sulfate, filtered and concentrated under reduced pressure. The residue was purified by flash column chromatography on silica gel (20% ethyl acetate in hexane) to afford 52.4 (36 g, 64%). MS(ES): m/z 414.3 and 416.3 [M+H]⁺, ¹H NMR (DMSO-d₆, 400 MHz): δ 7.75 (s, 1H), 7.68 (s, 1H), 7.10 (s, 1H), 5.69 (s, 2H), 3.99 (s, 3H), 3.91 (s, 3H), 3.57-3.52 (m, 2H), 0.90-0.84 (m, 2H), 0.02 (s, 9H).

Synthesis of compound 52.5. To a solution of 52.4 (36 g, 87.16 mmol) in DCM (360 mL) was added dropwise trifluoracetic acid (180 mL, 5V) at 0° C. and stirred at room temperature for 4 h. Most solvent was removed under reduced pressure. To the residue was added methanol (360 mL) and ammonia aqueous solution (180 mL). The reaction mixture was stirred reaction mixture was at room temperature for 3 h. The reaction mixture was concentrated under reduced pressure and the residue extracted with ethyl acetate. The combined organic layers were washed with brine, dried over anhydrous sodium sulfate, filtered and concentrated under reduced pressure. The residue was purified by flash column chromatography on silica gel (40% ethyl acetate in hexane) to afford 52.5 (20.2 g, 82%). MS(ES): m/z 284.2 and 286.3 [M+H]⁺, ¹H NMR (DMSO-d₆, 400 MHz): δ 12.31 (s, 1H), 8.01 (s, 1H), 7.72 (s, 1H), 7.29 (s, 1H), 3.84 (s, 3H), 3.79 (s, 3H).

Synthesis of compound 52.6. Compound 52.6 was prepared from compound 52.5 following the procedure described in the synthesis of compound 1.5. The product was purified by flash column chromatography on silica gel (80% ethyl acetate in hexane). MS(ES): m/z 299.1 and 300.9 [M+H]⁺, ¹H NMR (DMSO-d₆, 400 MHz): δ 7.99 (s, 1H), 7.70 (s, 1H), 7.35 (s, 1H), 6.35 (s, 2H), 3.83 (s, 3H), 3.80 (s, 3H).

Synthesis of compound 52.7. Compound 52.7 was prepared from compound 52.6 following the procedure described in the synthesis of compound 1.6. The product was purified by flash column chromatography on silica gel (20% to 100% ethyl acetate in hexane). MS(ES): m/z 396.1 and 398.1 [M+H]⁺.

Synthesis of compound 52.8. Compound 52.8 was prepared from compound 52.7 following the procedure described in the synthesis of compound 1.7. The product was used in the next step without purification. MS(ES): m/z 365 and 367 [M+H]⁺.

Synthesis of compound 52.9. Compound 52.9 was prepared from compound 52.8 following the procedure described in the synthesis of compound 1.8. The product was purified by flash column chromatography on silica gel (40% ethyl acetate in hexane). MS(ES): m/z 384.4 [M+H]⁺, ¹H NMR (DMSO-d₆, 400 MHz): δ 8.57 (s, 1H), 8.56 (s, 1H), 8.49 (s, 1H), 7.95 (s, 1H), 4.36-4.30 (m, 2H), 3.91 (s, 3H), 1.36-1.32 (m, 3H).

Synthesis of compound 52.10. Compound 52.10 was prepared from compound 52.9 and tert-butyl ((1R,3R)-3-aminocyclopentyl)carbamate following the procedure described in the synthesis of compound 1.9. The product was purified by flash column chromatography on silica gel (30% ethyl acetate in hexane). MS(ES): m/z 547.2 and 549.2 [M+H]⁺, ¹H NMR (DMSO-d₆, 400 MHz): δ 8.22 (s, 1H), 8.16 (s, 1H), 8.13 (s, 1H) 7.87 (s, 1H), 7.66 (d, J=7.2 Hz, 1H), 6.94 (d, J=6.4 Hz, 1H), 4.49-4.48 (m, 1H), 4.30-4.25 (m, 2H), 3.94-3.92 (m, 1H), 3.89 (s, 3H), 2.14-2.09 (m, 1H), 1.98-1.94 (m, 1H), 1.84-1.80 (m, 2H), 1.51-1.43 (m, 2H), 1.36 (s, 9H), 1.33-1.30 (m, 3H).

Synthesis of compound 52.11. Compound 52.11 was prepared from compound 52.10 and thieno[2,3-c]pyridin-2-ylboronic acid following the procedure described in the synthesis of compound 1.13. The product was purified by flash column chromatography on silica gel (50% ethyl acetate in hexane). MS(ES): m/z 602.62 [M+H]⁺.

Synthesis of compound 52.12. Compound 52.12 was prepared from compound 52.11 following the procedure described in the synthesis of compound 1.10. The product was used in the next step without purification. MS(ES): m/z 574.60 [M+H]⁺.

Synthesis of compound 52.13. Compound 52.13 was prepared from compound 52.12 following the procedure described in the synthesis of compound 1.11. The product was purified by flash column chromatography on silica gel (Combiflash®, 2.0% methanol in DCM). MS(ES): m/z 571.70 [M+H]⁺.

Synthesis of compound 52.14. Compound 52.14 was prepared from compound 52.13 following the procedure described in the synthesis of compound 1.12. The product was purified by flash column chromatography on silica gel (2.0% methanol in DCM). MS(ES): m/z 585.60 [M+H]⁺.

Synthesis of compound 52.15. Compound 52.15 was prepared from compound 52.14 following the procedure described in the synthesis of compound 1.14. The product was triturated with n-pentane and dried under reduced pressure. It was used in the next step without purification. MS(ES): m/z 485.58 [M+H]⁺.

Synthesis of compound I-52. Compound I-52 was prepared from compound 52.15 following the procedure described in the synthesis of compound I-1. The product was purified by flash column chromatography on silica gel (Combiflash®, 50% ethyl acetate in hexane). MS (ES): m/z 543.35 [M+H]⁺, ¹H-NMR (400 MHz, DMSO-d₆): δ 9.24 (s, 1H), 8.54 (d, J=4.8 Hz, 1H), 8.52 (s, 1H), 8.21 (s, 1H), 7.86 (d, J=5.6 Hz, 1H), 7.68 (s, 1H), 7.50 (s, 1H), 7.25 (s, 1H), 6.65 (bs, 1H), 3.92 (bs, 1H), 3.71 (s, 3H), 3.50 (s, 3H), 3.46 (s, 3H), 2.07 (bs, 1H), 1.97 (bs, 1H), 1.61 (bs, 1H), 1.39-1.36 (m, 1H), 1.08-1.09 (m, 1H), 0.85 (m, 0.5H), 0.69 (m, 0.5H), 0.01 (m, 1H).

### Example I-53: methyl ((1R,3R)-3-(9-(1H-indazol-6-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

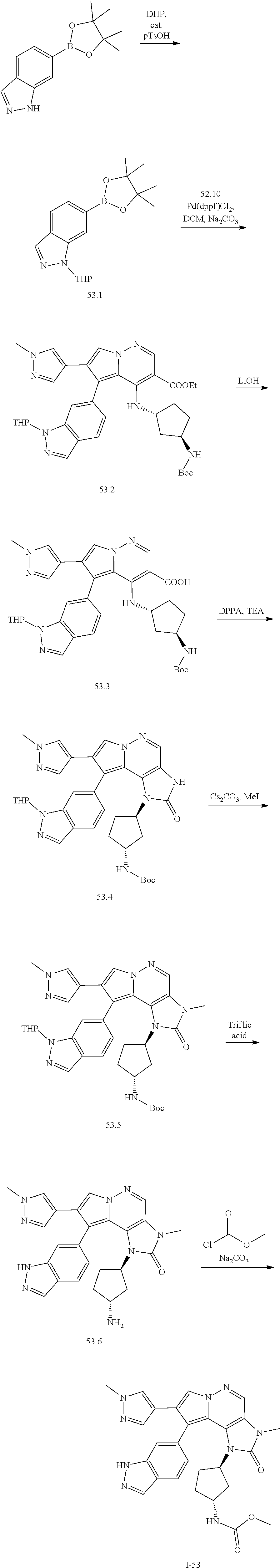

Synthesis of compound 53.1. To a solution of 6-(4,4,5,5-tetramethyl-1,3,2-dioxaborolan-2-yl)-1H-indazole (5.0 g, 0.205 mmol, 1.0 equiv) in DCM (3.0 mL) at room temperature was added p-toluene sulfonic acid (0.389 g, 0.020 mmol, 0.1 equiv) and 3,4-dihydropyran (2.58 g, 0.307 mmol, 1.5 equiv) and stirred for 15 h. It was transferred into ice-water and extracted with DCM. The combined organic layers were washed with brine, dried over anhydrous sodium sulfate, filtered and concentrated under reduced pressure. The residue was purified by flash column chromatography on silica gel (14% ethyl acetate in hexane) to afford 53.1 (5.0 g, 74%). MS(ES): m/z 329.21 [M+H]⁺.

Synthesis of compound 53.2. Compound 53.2 was prepared from compound 52.10 and 53.1 following the procedure described in the synthesis of compound 1.13. The product was purified by flash column chromatography on silica gel (50% ethyl acetate in hexane). MS(ES): m/z 669.34 [M+H]⁺.

Synthesis of compound 53.3. Compound 53.3 was prepared from compound 53.2 following the procedure described in the synthesis of compound 1.10. The product was used in the next step without purification. MS(ES): m/z 641.31 [M+H]⁺.

Synthesis of compound 53.4. Compound 53.4 was prepared from compound 53.3 following the procedure described in the synthesis of compound 1.11. The product was purified by flash column chromatography on silica gel (Combiflash®, 1.8% methanol in DCM). MS(ES): m/z 638.3 [M+H]⁺.

Synthesis of compound 53.5. Compound 53.5 was prepared from compound 53.4 following the procedure described in the synthesis of compound 1.12. The product was purified by flash column chromatography on silica gel (1.4% methanol in DCM). MS(ES): m/z 652.33 [M+H]⁺.

Synthesis of compound 53.6. Compound 53.6 was prepared from compound 53.5 following the procedure described in the synthesis of compound 1.14. The product was used in the next step without purification. MS(ES): m/z 468.22 [M+H]⁺.

Synthesis of I-53. Compound I-53 was prepared from compound 53.6 following the procedure described in the synthesis of compound I-1. The product was purified by flash column chromatography on silica gel (Combiflash®, 3.2% methanol in DCM). MS(ES): m/z 526.3 [M+H]⁺, ¹H-NMR (400 MHz, DMSO-d₆): δ 13.11 (d, 1H), 8.40 (d, J=2.0 Hz, 1H), 8.17 (s, 2H), 7.91 (d, J=8.0 Hz, 1H), 7.50 (d, J=8.0 Hz, 1H), 7.25 (d, J=6.0 Hz, 1H), 7.15-7.10 (m, 1H), 7.06-6.94 (m, 1H), 6.67-6.31 (m, 1H), 3.89 (m, 1H), 3.66 (s, 3H), 3.49-3.44 (m, 3H), 3.34 (s, 3H), 1.97 (m, 1H), 1.82-1.68 (m, 2H), 1.49 (m, 1H), 1.33-1.35 (m, 1H), 1.04-1.10 (m, 1H), 0.34 (s, 1H).

### Example I-54: methyl ((1R,3R)-3-(9-(7-fluoro-1H-indazol-5-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

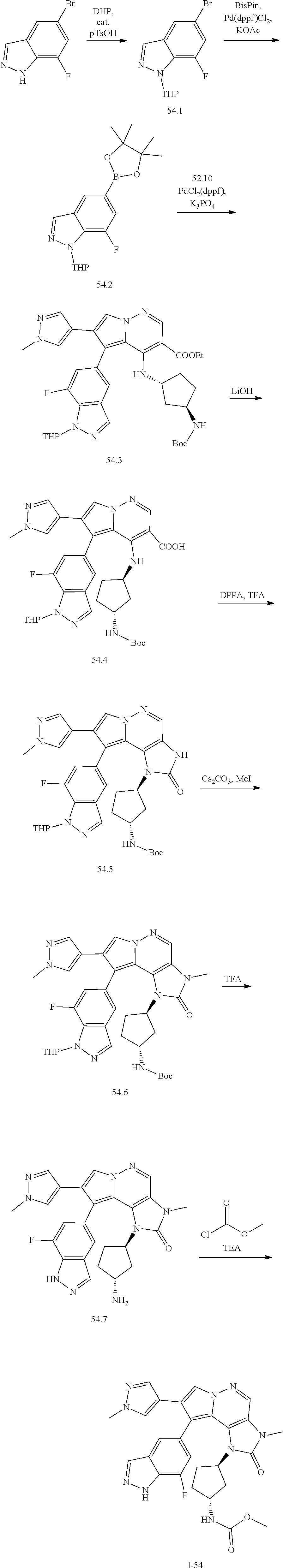

Synthesis of compound 54.1. Compound 54.1 was prepared from compound 5-bromo-7-fluoro-1H-indazole following the procedure described in the synthesis of compound 53.1. The product was purified by flash column chromatography on silica gel (Combiflash®, 10%. MS(ES): m/z 300.3 [M+H]⁺.

Synthesis of compound 54.2. A mixture of 54.1 (14.5 g, 48.47 mmol, 1.0 equiv), bis(pinacolato)diboron (18.46 g, 72.70 mmol, 1.5 equiv) and potassium acetate (14.27 g, 145.4 mmol, 3.0 equiv) in 1,4-dioxane (100 mL) was purged by bubbling through a stream of argon for 15 min. [1,1′-Bis(diphenylphosphino) ferrocene]dichloropalladium (II) (3.54 g, 4.8 mmol, 0.1 equiv) was added. The reaction mixture was stirred at 100° C. for 2 h. It was cooled to room temperature, transferred into water and extracted with ethyl acetate. The combined organic layers were washed with brine, dried over anhydrous sodium sulfate, filtered and concentrated under reduced pressure. The residue was purified by flash column chromatography on silica gel (Combiflash®, 32% ethyl acetate in hexane) to afford 54.2 (12.8 g, 76%). MS(ES): m/z 347.41 [M+H]⁺.

Synthesis of compound 54.3. A mixture of 52.10 (0.375 g, 0.685 mmol, 1.0 equiv), 54.2 (0.711 g, 2.05 mmol, 3 equiv) and potassium phosphate tribasic (0.290 g, 1.4 mmol, 2 equiv) in 1,4-dioxane (6.0 mL) and water (2 mL) was degassed by bubbling through a stream of argon for 10 min. [1,1′-Bis(diphenylphosphino)ferrocene]dichloropalladium (II) (0.050 g, 0.068 mmol, 0.1 equiv) was added and again degassed by bubbling through a stream of argon for 5 min. The reaction mixture was stirred at 100° C. for 2 h. It was cooled to room temperature, filtered through a pad of Celite® and rinsed with ethyl acetate. The filtrate was transferred into water and extracted with ethyl acetate. The combined organic layers were washed with brine, dried over anhydrous sodium sulfate, filtered and concentrated under reduced pressure. The residue was purified by flash column chromatography on silica gel (2.5% methanol in DCM) to afford 54.3 (0.210 g, 45%). MS(ES): m/z 687.8 [M+H]⁺.

Synthesis of compound 54.4. Compound 54.4 was prepared from compound 54.3 following the procedure described in the synthesis of compound 1.10. The product was triturated with pentane, dried under vacuum and used in the next step without purification. MS(ES): m/z 659.7 [M+H]⁺.

Synthesis of compound 54.5. Compound 54.5 was prepared from compound 54.4 following the procedure described in the synthesis of compound 1.11. The product was purified by flash column chromatography on silica gel (Combiflash®, 1.8% methanol in DCM). MS(ES): m/z 656.8 [M+H]⁺.

Synthesis of compound 54.6. Compound 54.6 was prepared from compound 54.5 following the procedure described in the synthesis of compound 1.12. The product was purified by flash column chromatography on silica gel (1.6% methanol in DCM). MS(ES): m/z 670.8 [M+H]⁺.

Synthesis of compound 54.7. Compound 54.7 was prepared from compound 54.6 following the procedure described in the synthesis of compound 1.14. The product was used in the next step without purification. MS(ES): m/z 486.7 [M+H]⁺.

Synthesis of compound I-54. Compound I-54 was prepared from compound 54.7 following the procedure described in the synthesis of compound I-1. The product was purified by flash column chromatography on silica gel (Combiflash®, 3.2% methanol in DCM). MS (ES): m/z 544.3 [M+H]⁺, ¹H-NMR (400 MHz, DMSO-d₆): δ 13.76 (s, 1H), 8.41 (s, 1H), 8.21-8.20 (m, 1H), 8.14 (s, 1H), 7.64 (d, J=6.4 Hz, 1H), 7.26 (s, 1H), 7.20 (d, J=11.2 Hz, 1H), 7.02-6.98 (m, 1H), 6.77 (m, 0.5H), 6.52 (m, 0.5H), 4.04 (bs, 1H), 3.67 (s, 3H), 3.46 (s, 3H), 3.35 (s, 3H), 2.01-1.99 (m, 2H), 1.75 (bs, 1H), 1.58-1.51 (m, 1H), 1.00-0.85 (m, 2H), 0.24 (m, 0.5H), −0.02-−0.04 (m, 0.5H).

### Example I-55: (1R,2R)-2-fluoro-N-((1s,4S)-4-(8-(3-methoxy-1-methyl-1H-pyrazol-4-yl)-3-methyl-9-(1-methyl-1H-indazol-5-yl)-2-oxo-2,3-dihydro-1H-imidazo[4,5-d]pyrrolo[1,2-b]pyridazin-1-yl)-1-methylcyclohexyl)cyclopropane-1-carboxamide

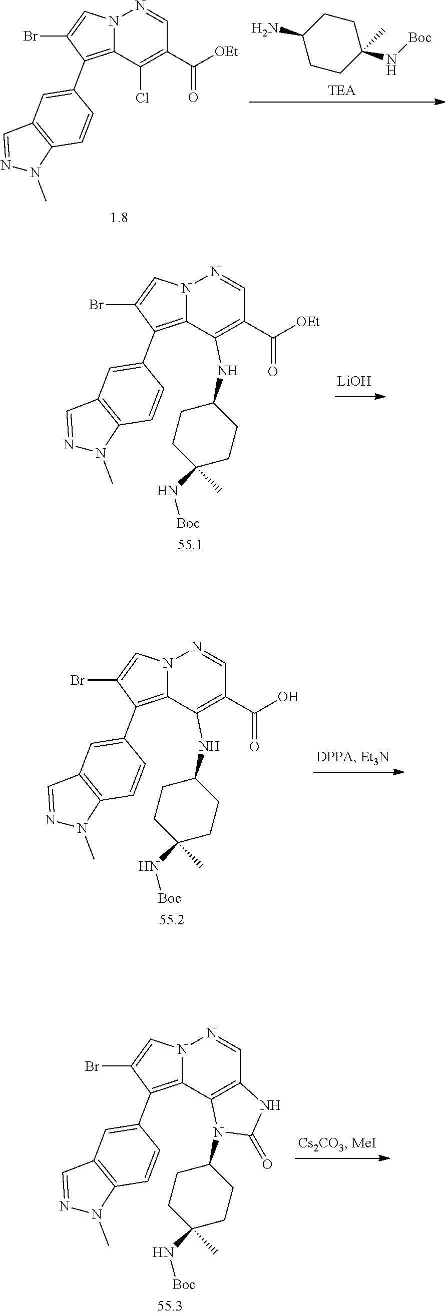

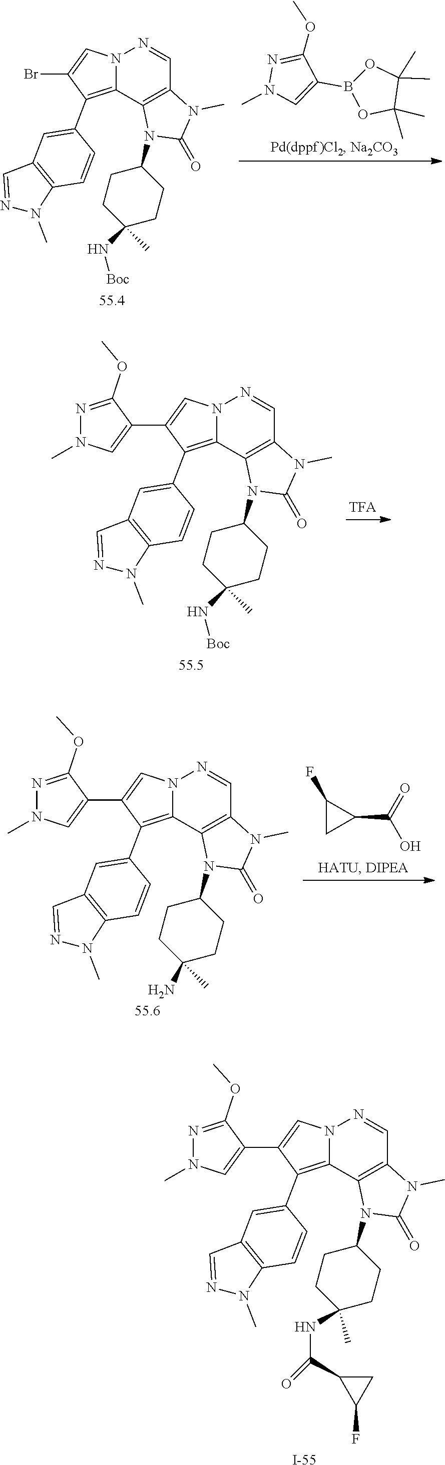

Synthesis of compound 55.1. Compound 55.1 was prepared from compound 1.8 and tert-butyl ((1s,4s)-4-amino-1-methylcyclohexyl)carbamate following the procedure described in the synthesis of compound 1.9. The product was purified by flash column chromatography on silica gel (Combiflash®, 50% ethyl acetate in hexane). MS(ES): m/z 625.3 and 627.4 [M+H]⁺.

Synthesis of compound 55.2. Compound 55.2 was prepared from compound 55.1 following the procedure described in the synthesis of compound 1.10. The product was triturated with pentane, dried under vacuum and used in the next step without purification. MS(ES): m/z 597.35 and 599.35 [M+H]⁺.

Synthesis of compound 55.3. Compound 55.3 was prepared from compound 55.2 following the procedure described in the synthesis of compound 1.11. The product was purified by flash column chromatography on silica gel (Combiflash®, 1.8% methanol in DCM). MS(ES): m/z 594.2 and 596.4 [M+H]⁺.

Synthesis of compound 55.4. Compound 55.4 was prepared from compound 55.3 following the procedure described in the synthesis of compound 1.12. The product was purified by flash column chromatography on silica gel (1.4% methanol in DCM). MS(ES): m/z 607.1 and 609.5 [M+H]⁺.

Synthesis of compound 55.5. Compound 55.5 was prepared from compound 55.4 and 3-methoxy-1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (Combiflash®, 3.2% methanol in DCM). MS(ES): m/z 640.3 [M+H]⁺.

Synthesis of compound 55.6. Compound 55.6 was prepared from compound 55.5 following the procedure described in the synthesis of compound 1.14. The product was triturated with pentane and dried under reduced pressure. It was used in the next step without purification. MS(ES): m/z 540.3 [M+H]⁺.

Synthesis of I-55. Compound I-55 was prepared from compound 55.6 and (1R,2R)-2-fluorocyclopropane-1-carboxylic acid following the procedure described in the synthesis of compound I-45. The product was purified by silica gel flash column chromatography on silica gel (4.8% methanol in DCM). MS(ES): m/z 626.3 [M+H]⁺, ¹H-NMR (400 MHz, DMSO-d₆): δ 8.37 (s, 1H), 8.11 (s, 1H), 8.05 (s, 1H) 7.83 (d, J=8.4 Hz, 1H), 7.76 (s, 1H), 7.37 (d, J=8.4 Hz, 1H), 7.26 (s, 1H), 6.11 (s, 1H), 4.11 (s, 3H), 3.85 (s, 3H), 3.39 (s, 3H), 3.35 (s, 3H), 2.45-2.32 (m, 2H), 2.32-1.78 (m, 1H), 1.76-1.74 (m, 1H), 1.33-1.23 (m, 1H), 1.22-1.14 (m, 1H), 0.97-0.93 (m, 1H), 0.92-0.90 (m, 3H), 0.67 (s, 3H).

### Example I-56: methyl ((1s,4s)-4-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclohexyl)carbamate

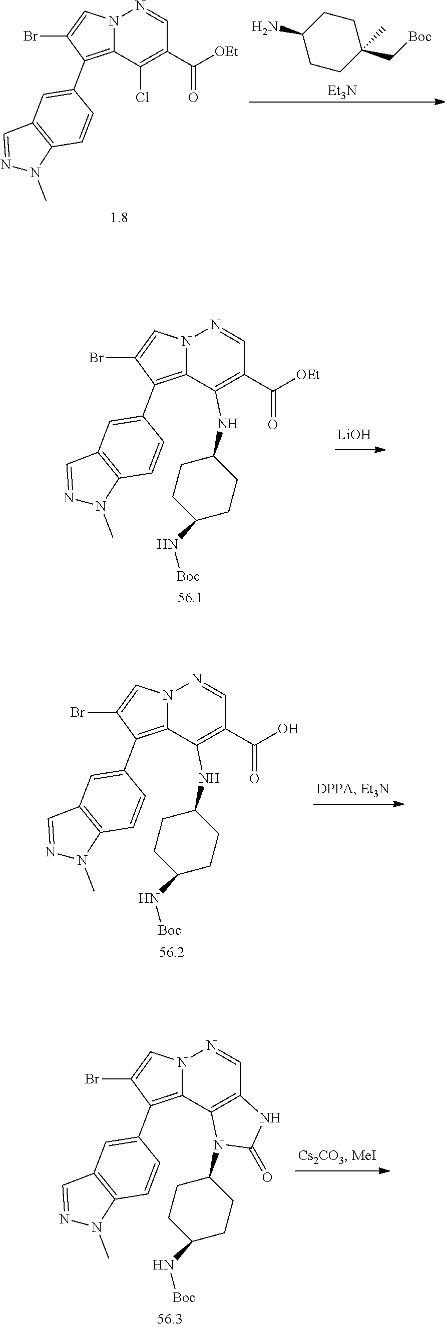

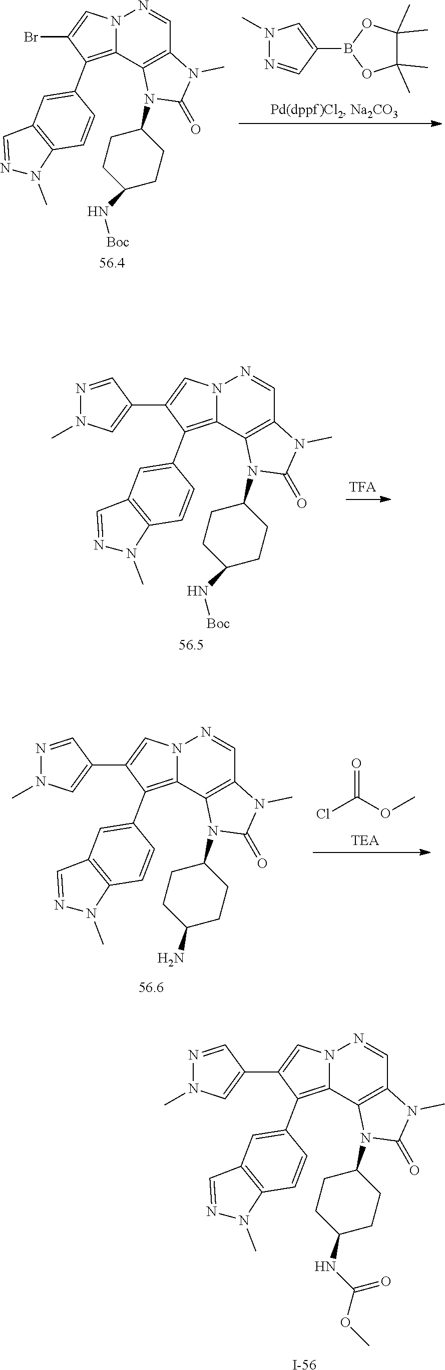

Synthesis of compound 56.1. Compound 56.1 was prepared from compound 1.8 and tert-butyl ((1s,4s)-4-aminocyclohexyl)carbamate following the procedure described in the synthesis of compound 1.9. The product was purified by flash column chromatography on silica gel (15% ethyl acetate in n-hexane). MS(ES): m/z 611.54 and 613.54 [M+H]⁺.

Synthesis of compound 56.2. Compound 56.2 was prepared from compound 56.1 following the procedure described in the synthesis of compound 1.10. The product was triturated with pentane, dried under vacuum and used in the next step without purification. MS (ES): m/z 583.16 and 585.16 [M+H]⁺.

Synthesis of compound 56.3. Compound 56.3 was prepared from compound 56.2 following the procedure described in the synthesis of compound 1.11. The product was purified by flash column chromatography on silica gel (2.1% methanol in DCM). MS(ES): m/z 580.1 and 582.1 [M+H]⁺.

Synthesis of compound 56.4. Compound 56.4 was prepared from compound 56.3 following the procedure described in the synthesis of compound 1.12. The product was purified by flash column chromatography on silica gel (2.5% methanol in DCM). MS(ES): m/z 594.6 and 595.6 [M+H]⁺.

Synthesis of compound 56.5. Compound 56.5 was prepared from compound 56.4 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (16% ethyl acetate in n-hexane). MS (ES): m/z 596.7 [M+H]⁺.

Synthesis of compound 56.6. Compound 56.6 was prepared from compound 56.5 following the procedure described in the synthesis of compound 1.14. The product was used in the next step without purification.

Synthesis of compound I-56. Compound I-56 was prepared from compound 56.6 following the procedure described in the synthesis of compound I-1. The product was purified by flash column chromatography on silica gel (3.8% methanol in DCM). MS (ES): m/z 554.3 [M+H]⁺, ¹H NMR (DMSO-d₆, 400 MHz): δ 8.39 (s, 1H), 8.14 (s, 1H), 8.09 (s, 1H), 7.78 (t, J=14.8 Hz, 2H), 7.38 (d, J=8.4 Hz, 1H), 7.21 (s, 1H), 6.91 (s, 1H), 6.65 (s, 1H), 4.13 (s, 3H), 3.65 (s, 3H), 3.46 (s, 3H), 3.09 (s, 1H), 2.90 (s, 1H), 2.40-2.20 (m, 3H), 1.46 (s, 1H), 1.23 (d, J=3.2 Hz, 2H), 0.96 (t, J=12.0 Hz, 3H), 0.25 (s, 1H).

### Example I-57: methyl ((1R,3R)-3-(3-methyl-8-(1-methyl-1H-pyrazol-4-yl)-2-oxo-9-(pyrazolo[1,5-a]pyridin-5-yl)-2,3-dihydro-1H-imidazo[4,5-d]pyrrolo[1,2-b]pyridazin-1-yl)cyclopentyl)carbamate

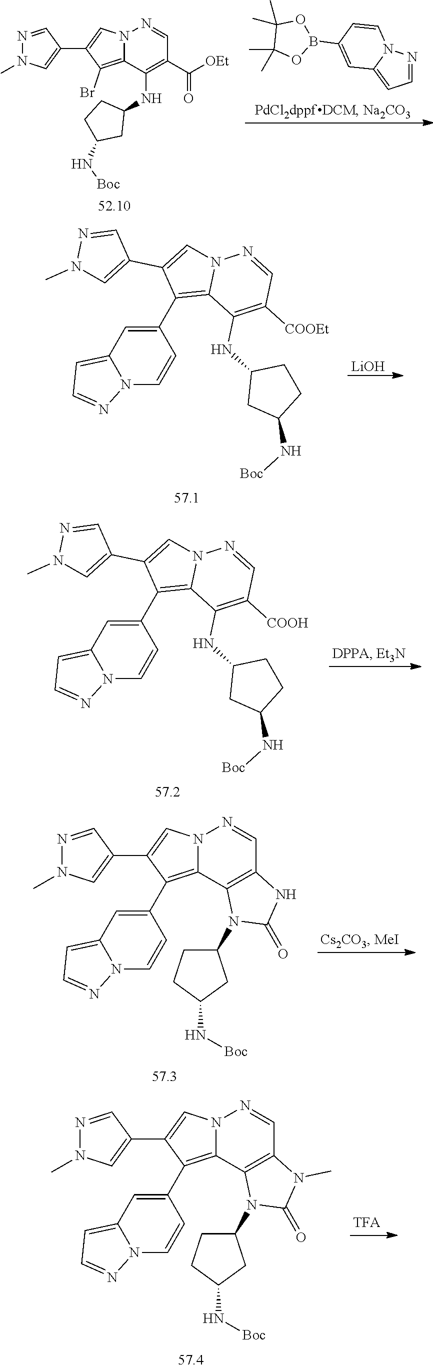

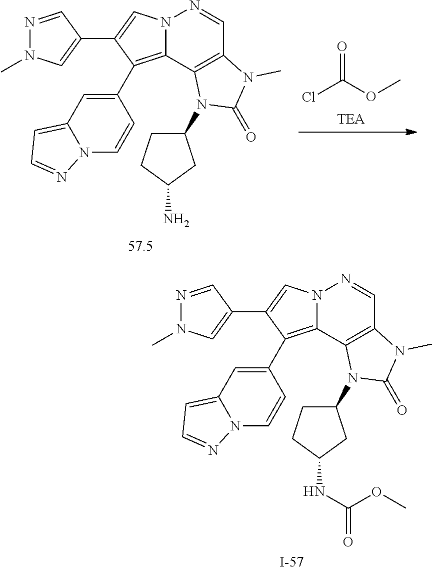

Synthesis of compound 57.1. Compound 57.1 was prepared from compound 52.10 and 5-(4,4,5,5-tetramethyl-1,3,2-dioxaborolan-2-yl)pyrazolo[1,5-a]pyridine following the procedure described in the synthesis of compound 1.13. The product was purified by flash column chromatography on silica gel (55% ethyl acetate in hexane). MS(ES): m/z 585.72 [M+H]⁺.

Synthesis of compound 57.2. Compound 57.2 was prepared from compound 57.1 following the procedure described in the synthesis of compound 1.10. The product was used in the next step without purification. MS(ES): m/z 557.70 [M+H]⁺.

Synthesis of compound 57.3. Compound 57.3 was prepared from compound 57.2 following the procedure described in the synthesis of compound 1.11. The product was purified by flash column chromatography on silica gel (Combiflash®, 1.6% methanol in DCM). MS(ES): m/z 554.68 [M+H]⁺.

Synthesis of compound 57.4. Compound 57.4 was prepared from compound 57.3 following the procedure described in the synthesis of compound 1.12. The product was purified by flash column chromatography on silica gel (1.2% methanol in DCM). MS(ES): m/z 568.70 [M+H]⁺.

Synthesis of compound 57.5. Compound 57.5 was prepared from compound 57.4 following the procedure described in the synthesis of compound 1.14. The product was triturated with pentane and dried under reduced pressure. It was used in the next step without purification. MS(ES): m/z 468.6 [M+H]⁺.

Synthesis of I-57. Compound I-57 was prepared from compound 57.5 following the procedure described in the synthesis of compound I-1. The product was purified by flash column chromatography on silica gel (Combiflash®, 3.8% methanol in DCM). MS(ES): m/z 526.2 [M+H]⁺, ¹H NMR (DMSO-d₆, 400 MHz): δ 8.75 (s, 1H), 8.43 (s, 1H), 8.15 (s, 1H), 8.08 (s, 1H), 7.72 (s, 1H), 7.45 (s, 1H), 7.25 (s, 1H), 6.91-6.85 (m, 2H), 6.62 (s, 1H), 3.93-3.82 (m, 2H), 3.71 (s, 3H), 3.47 (s, 3H), 3.40 (s, 3H), 2.08-205 (m, 1H), 1.79-1.60 (m, 2H), 1.56-1.48 (m, 1H), 1.11-1.05 (m, 2H).

### Example I-58: methyl ((1R,3R)-3-(3-methyl-8-(1-methyl-1H-pyrazol-4-yl)-9-(4-methyl-3,4-dihydro-2H-benzo[b][1,4]oxazin-7-yl)-2-oxo-2,3-dihydro-1H-imidazo[4,5-d]pyrrolo[1,2-b]pyridazin-1-yl)cyclopentyl)carbamate

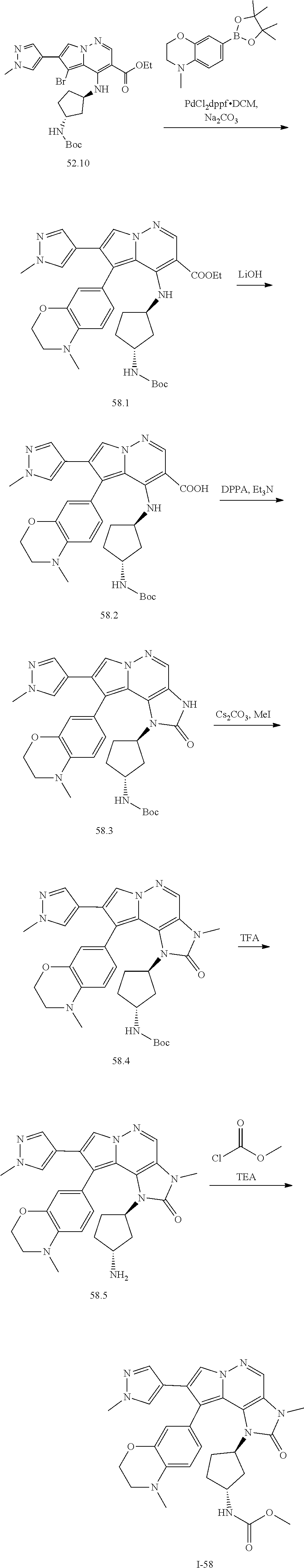

Synthesis of compound 58.1. Compound 58.1 was prepared from compound 52.10 and 4-methyl-7-(4,4,5,5-tetramethyl-1,3,2-dioxaborolan-2-yl)-3,4-dihydro-2H-benzo[b][1,4]oxazine following the procedure described in the synthesis of compound 1.13. The product was purified by flash column chromatography on silica gel (50% ethyl acetate in hexane). MS(ES): m/z 616.82 [M+H]⁺.

Synthesis of compound 58.2. Compound 58.2 was prepared from compound 58.1 following the procedure described in the synthesis of compound 1.10. The product was used in the next step without purification. MS(ES): m/z 588.70 [M+H]⁺.

Synthesis of compound 58.3. Compound 58.3 was prepared from compound 58.2 following the procedure described in the synthesis of compound 1.11. The product was purified by flash column chromatography on silica gel (Combiflash®, 1.8% methanol in DCM). MS(ES): m/z 585.71 [M+H]⁺.

Synthesis of compound 58.4. Compound 58.4 was prepared from compound 58.3 following the procedure described in the synthesis of compound 1.12. The product was purified by flash column chromatography on silica gel (1.4% methanol in DCM). MS(ES): m/z 599.68 [M+H]⁺.

Synthesis of compound 58.5. Compound 58.5 was prepared from compound 58.4 following the procedure described in the synthesis of compound 1.14. The product was triturated with pentane and dried under reduced pressure. It was used in the next step without purification. MS(ES): m/z 499.60 [M+H]⁺.

Synthesis of I-58. Compound I-58 was prepared from compound 58.5 following the procedure described in the synthesis of compound I-1. The product was purified by flash column chromatography on silica gel (Combiflash®, 4.2% methanol in DCM). MS(ES): m/z 558.38 [M+H]⁺, ¹H-NMR (400 MHz, DMSO-d₆): δ 8.34 (s, 1H), 8.07 (s, 1H), 7.38 (d, J=4.4 Hz, 1H), 7.14 (d, J=11.2 Hz, 1H), 6.86-6.78 (m, 3H), 6.62 (d, J=1.6 Hz, 1H), 4.33-4.24 (m, 2H), 4.08-3.92 (m, 2H), 3.74 (s, 3H), 3.51 (s, 3H), 3.35-3.33 (m, 4H), 2.93 (d, J=12.4 Hz, 3H), 2.12-2.09 (m, 1H), 1.97-1.90 (m, 2H), 1.53-1.49 (m, 1H), 1.34-1.23 (m, 1H), 0.88-0.84 (m, 1H).

### Example I-59: methyl ((1R,3R)-3-(8-(3,6-dihydro-2H-pyran-4-yl)-3-methyl-2-oxo-9-(1H-pyrazolo[3,4-b]pyridin-6-yl)-2,3-dihydro-1H-imidazo[4,5-d]pyrrolo[1,2-b]pyridazin-1-yl)cyclopentyl)carbamate

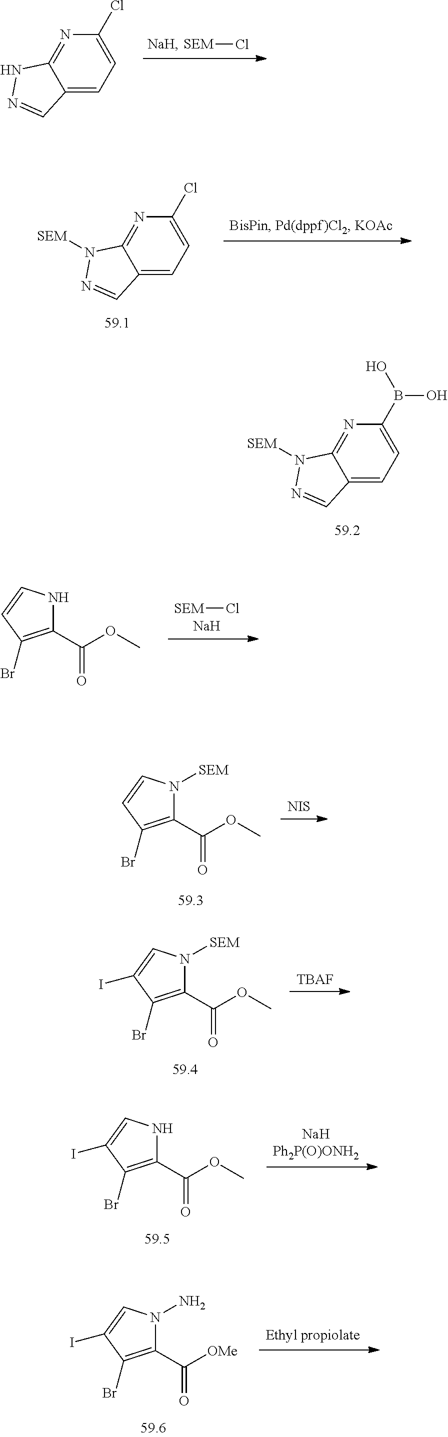

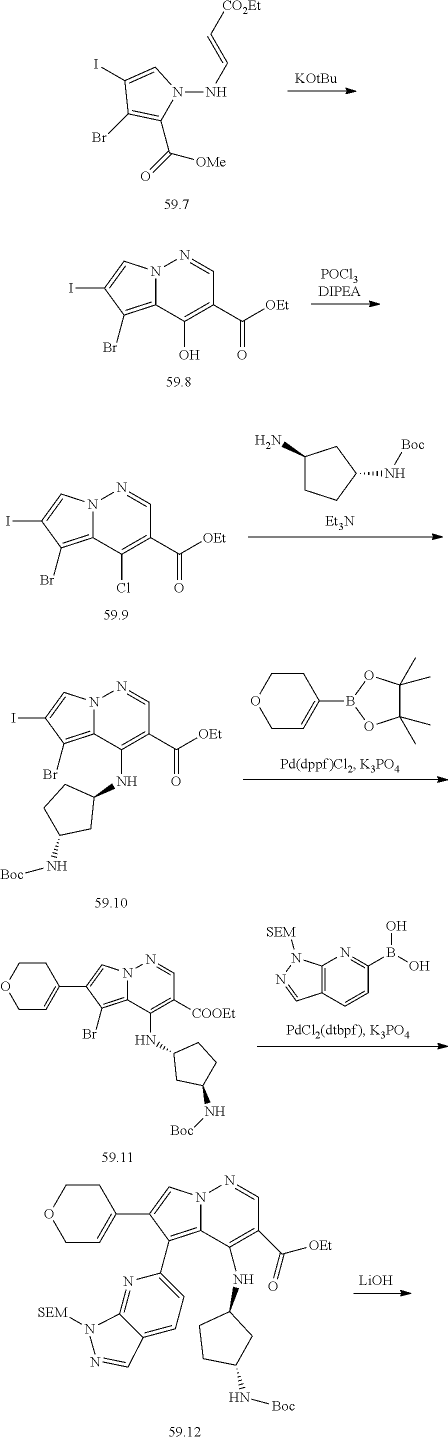

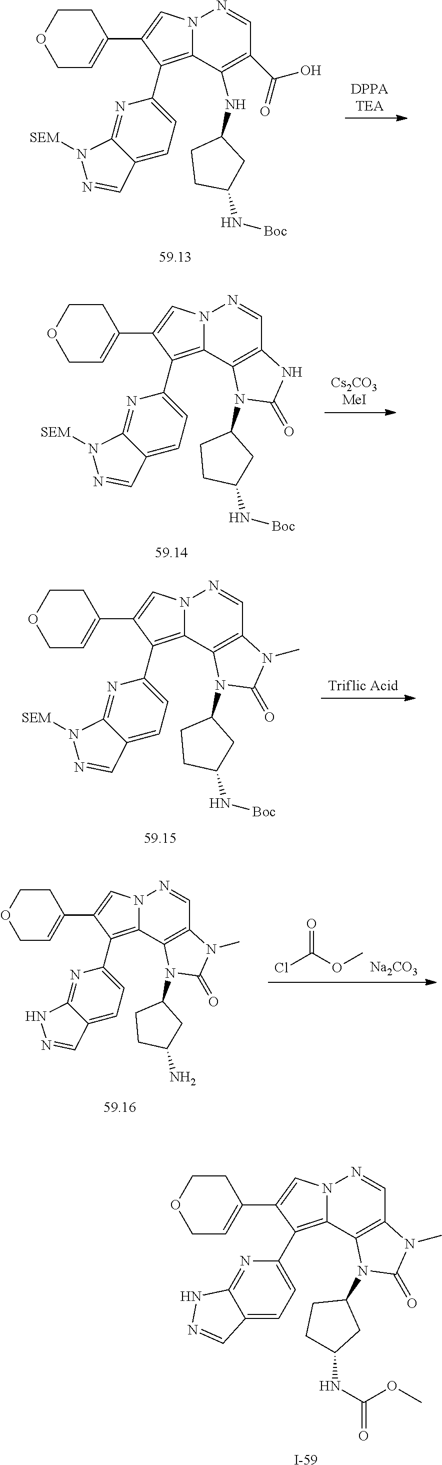

Synthesis of compound 59.1. To a solution of 6-chloro-1H-pyrazolo[3,4-b]pyridine (2.0 g, 13.07 mmol, 1.0 equiv) in DMF (40 mL) was added sodium hydride (1.04 g, 26.14 mmol, 2.0 equiv) at 0° C. and stirred for 30 min. 2-(trimethylsilyl)ethoxymethyl chloride (3.25 g, 19.60 mmol, 1.5 equiv) was added at same temperature and the reaction mixture was stirred at room temperature for 2 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 10% ethyl acetate in hexane) to afford 59.1 (2.1 g, 57%). MS(ES): m/z 284.1 [M+H]⁺.

Synthesis of compound 59.2. A mixture of 59.1. (0.5 g, 1.77 mmol, 1.0 equiv), bis(pinacolato) diboron (0.89 g, 3.53 mmol, 1.0 equiv) and potassium acetate (0.52 g, 5.30 mmol, 3 equiv) in DMF (20 mL) was degassed by bubbling through a stream of argon for 10 min. (1,1′-Bis(diphenylphosphino)ferrocene) palladium (II) dichloride (0.13 g, 0.17 mmol, 0.1 equiv) was added and degassed for 5 min. The reaction mixture was stirred at 100° C. for 16 h. It was transferred into water and extracted with ethyl acetate. The combined organic layers were washed with brine, dried over anhydrous sodium sulfate, filtered and concentrated under reduced pressure to afford 59.2 (0.49 g, 94.87%). The residue used in the next step directly without purification. MS(ES): m/z 294.2 [M+H]⁺.

Synthesis of compound 59.3. To a solution of methyl 3-bromo-1H-pyrrole-2-carboxylate (50 g, 245.07 mmol, 1.0 equiv) in DMF (500 mL) was added sodium hydride (60% dispersion in mineral oil, 23.5 g, 490.14 mmol, 2 equiv) in small portions at 0° C. and stirred for 30 min. To the suspension was added 2-(trimethylsilyl) ethoxymethyl chloride (61.28 g, 367.61 mmol, 1.5 equiv). The reaction mixture was stirred at 0° C. for 2 h. It was quenched with ice-water and extract with ethyl acetate. The combined organic layers were washed with brine, dried over anhydrous sodium sulfate, filtered and concentrated under reduced pressure. The residue was purified by flash column chromatography on silica gel (15% ethyl acetate in hexane) to afford 59.3 (62 g, 76%). MS(ES): m/z 334.5 & 336.6 [M+H]⁺.

Synthesis of compound 59.4. To a solution of 59.3 (62 g, 185.47 mmol, 1.0 equiv) in DMF (1200 mL) was added N-Iodosuccinimide (125.18 g, 556.41 mmol, 3 equiv) in small portions. The reaction mixture was stirred at room temperature for 24 h. It was cooled to room temperature, and quenched with 10% sodium thiosulfate solution, extracted with ethyl acetate. The combined organic layers were washed with brine, dried over anhydrous sodium sulfate, filtered and concentrated under reduced pressure. The residue was purified by flash column chromatography on silica gel (10% ethyl acetate in hexane) to afford 59.4 (62 g, 70%). MS(ES): m/z 460.2 & 462.4 [M+H]⁺.

Synthesis of compound 59.5. To a solution of 59.4 (60 g, 130.38 mmol, 1.0 equiv) in THF (500 mL) was added tetrabutylammonium fluoride solution (1.0 M in THF, 440.6 mL, 440.6 mmol, 2 equiv) in small portions at 0° C. and stirred for 30 min. The reaction mixture was stirred at 70° C. for 12 h. It was quenched with ice-water and extract with ethyl acetate. The combined organic layers were washed with brine, dried over anhydrous sodium sulfate, filtered and concentrated under reduced pressure. The residue was purified by flash column chromatography on silica gel (30% ethyl acetate in hexane) to afford 59.5 (28 g, 65%). MS(ES): m/z 329.8 & 331.5 [M+H]⁺.

Synthesis of compound 59.6. Compound 59.6 was prepared from compound 59.5 following the procedure described in the synthesis of compound 1.5. The product was purified by flash column chromatography on silica gel (35% ethyl acetate in hexane). MS(ES): m/z 344.9 & 345.9 [M+H]⁺.

Synthesis of compound 59.7. Compound 59.7 was prepared from compound 59.6 following the procedure described in the synthesis of compound 1.6. The product was purified by flash column chromatography on silica gel (20% to 100% ethyl acetate in hexane). MS(ES): m/z 443.31 & 445.24 [M+H]⁺.

Synthesis of compound 59.8. Compound 59.8 was prepared from compound 59.7 following the procedure described in the synthesis of compound 1.7. The product used in the next step without purification. MS(ES): m/z 410.8 & 412.9 [M+H]⁺.

Synthesis of compound 59.9. Compound 59.9 was prepared from compound 59.8 following the procedure described in the synthesis of compound 1.8. The product was purified by flash column chromatography on silica gel (25% ethyl acetate in hexane). MS(ES): m/z 429.2 & 431.5 [M+H]⁺.

Synthesis of compound 59.10. Compound 59.10 was prepared from compound 59.9 and tert-butyl ((1R,3R)-3-aminocyclopentyl)carbamate following the procedure described in the synthesis of compound 1.9. The product was purified by flash column chromatography on silica gel (30% ethyl acetate in hexane). MS(ES): m/z 593.2 & 595.2 [M+H]⁺.

Synthesis of compound 59.11. Compound 59.11 was prepared from compound 59.10 and 2-(3,6-dihydro-2H-pyran-4-yl)-4,4,5,5-tetramethyl-1,3,2-dioxaborolane following the procedure described in the synthesis of compound 54.3. The product was purified by flash column chromatography on silica gel (50% ethyl acetate in hexane). MS(ES): m/z 549.34 and 551.34 [M+H]⁺.

Synthesis of compound 59.12. A mixture of 59.11 (0.400 g, 0.73 mmol, 1.0 equiv), (1-((2-(trimethylsilyl)ethoxy)methyl)-1H-pyrazolo[3,4-b]pyridin-6-yl)boronic acid (0.426 g, 1.46 mmol, 2.0 equiv), and tripotassium phosphate (0.386 g, 1.82 mmol, 2.5 equiv) in 1,4-dioxane (9 mL) and water (1 mL) was degassed by bubbling through a stream of argon for 10 min. [1,1′-Bis(di-tert-butylphosphino)ferrocene]dichloropalladium (II) (0.095 g, 0.145 mmol, 0.1 equiv) was added and degassed for 5 min. The reaction mixture was stirred at room temperature for 1 h. It was filtered through a pad of Celite® and rinsed with ethyl acetate. The filtrate was transferred into water and extracted with ethyl acetate. The combined organic layers were washed with brine, dried over anhydrous sodium sulfate, filtered and concentrated under reduced pressure. The residue was purified by column chromatography (50% ethyl acetate in hexane) to afford 59.12 (0.320 g, 61%). MS(ES): m/z 719.2 [M+H]⁺.

Synthesis of compound 59.13. Compound 59.13 was prepared from compound 59.12 following the procedure described in the synthesis of compound 1.10. The product was triturated with pentane, dried under vacuum and used in the next step without purification. MS(ES): m/z 690.1 [M+H]⁺.

Synthesis of compound 59.14. Compound 59.14 was prepared from compound 59.13 following the procedure described in the synthesis of compound 1.11. The product was purified by flash column chromatography on silica gel (Combiflash®, 1.8% methanol in DCM). MS(ES): m/z 687.3 [M+H]⁺.

Synthesis of compound 59.15. Compound 59.15 was prepared from compound 59.14 following the procedure described in the synthesis of compound 1.12. The product was purified by flash column chromatography on silica gel (1.4% methanol in DCM). MS(ES): m/z 701. 2 [M+H]⁺.

Synthesis of compound 59.16. Compound 59.16 was prepared from compound 59.15 following the procedure described in the synthesis of compound 21.8. MS(ES): m/z 471.2 [M+H]⁺. The product was used in the next step without purification.

Synthesis of I-59. Compound I-59 was prepared from compound 59.15 following the procedure described in the synthesis of compound I-1. The product was purified by flash column chromatography on silica gel (Combiflash®, 3.7% methanol in DCM). MS(ES): m/z 529.2 [M+H]⁺, ¹H-NMR (400 MHz, DMSO-d₆): δ 13.64 (s, 1H), 8.47 (s, 1H), 8.31-8.29 (m, 1H), 8.19 (s, 1H), 7.98 (s, 1H), 7.29 (d, J=8.0 Hz, 1H), 6.59-6.57 (m, 1H), 5.31 (s, 1H), 3.90 (s, 3H), 3.64-3.63 (m, 2H), 3.46 (s, 3H), 2.15 (m, 2H), 2.08-2.07 (m, 2H), 1.82-1.80 (m, 2H), 1.57-1.55 (m, 1H), 1.35-1.33 (m, 1H), 1.20-1.17 (m, 2H), 1.00-0.84 (m, 2H).

### Example I-60: methyl ((1R,3R)-3-(8-(5,6-dihydro-4H-pyrrolo[1,2-b]pyrazol-3-yl)-9-(imidazo[1,5-a]pyridin-7-yl)-3-methyl-2-oxo-2,3-dihydro-1H-imidazo[4,5-d]pyrrolo[1,2-b]pyridazin-1-yl)cyclopentyl)carbamate

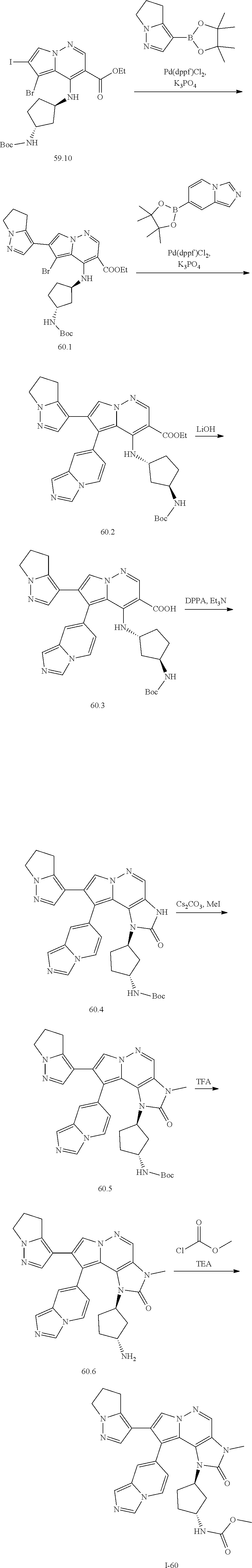

Synthesis of compound 60.1. Compound 60.1 was prepared from compound 59.10 and 3-(4,4,5,5-tetramethyl-1,3,2-dioxaborolan-2-yl)-5,6-dihydro-4H-pyrrolo[1,2-b]pyrazole following the procedure described in the synthesis of compound 54.3. The product was purified by flash column chromatography on silica gel (55% ethyl acetate in hexane). MS(ES): m/z 573.5 & 575.5 [M+H]⁺.

Synthesis of compound 60.2. Compound 60.2 was prepared from compound 60.1 and 7-(4,4,5,5-tetramethyl-1,3,2-dioxaborolan-2-yl)imidazo[1,5-a]pyridine following the procedure described in the synthesis of compound 54.3. The product was purified by flash column chromatography on silica gel (2.5% methanol in DCM). MS(ES): m/z 611.8 [M+H]⁺.

Synthesis of compound 60.3. Compound 60.3 was prepared from compound 60.2 following the procedure described in the synthesis of compound 1.10. The product was triturated with pentane, dried under vacuum and used in the next step without purification. MS(ES): m/z 583.7 [M+H]⁺.

Synthesis of compound 60.4. Compound 60.4 was prepared from compound 60.3 following the procedure described in the synthesis of compound 1.11. The product was purified by flash column chromatography on silica gel (Combiflash®, 2.0% methanol in DCM). MS(ES): m/z 580.8 [M+H]⁺.

Synthesis of compound 60.5. Compound 60.5 was prepared from compound 60.4 following the procedure described in the synthesis of compound 1.12. The product was purified by flash column chromatography on silica gel (1.8% methanol in DCM). MS(ES): m/z 594.8

Synthesis of compound 60.6. Compound 60.6 was prepared from compound 60.5 following the procedure described in the synthesis of compound 1.14. The product was triturated with n-pentane and dried under reduced pressure. It was used in the next step without purification. MS(ES): m/z 494.7 [M+H]⁺.

Synthesis of compound I-60. Compound I-60 was prepared from compound 60.6 following the procedure described in the synthesis of compound I-1. The product was purified by flash column chromatography on silica gel (Combiflash®, 3.2% methanol in DCM). MS (ES): m/z 552.3 [M+H]⁺, ¹H-NMR (400 MHz, DMSO-d₆): δ 8.43-8.41 (m, 3H), 7.97 (s, 1H), 7.57 (s, 1H), 7.37 (s, 1H), 7.00-6.94 (m, 1H), 6.82 (bs, 0.5H), 6.64 (d, J=6.8 Hz, 1H), 6.49 (s, 0.5H), 4.39-4.40 (m, 1H), 4.02-3.99 (m, 3H), 3.49 (s, 3H), 3.36 (s, 3H), 2.80 (bs, 2H), 2.15 (bs, 1H), 1.97-1.83 (m, 3H), 1.55 (bs, 2H), 0.85-0.83 (m, 1H), 0.69 (m, 0.5H), 0.06 (m, 0.5H).

### Example I-61: 1-((1s,4s)-4-hydroxy-4-methylcyclohexyl)-3-methyl-9-(1-methyl-1H-indazol-5-yl)-8-(1-methyl-1H-pyrazol-4-yl)-1,3-dihydro-2H-imidazo[4,5-d]pyrrolo[1,2-b]pyridazin-2-one

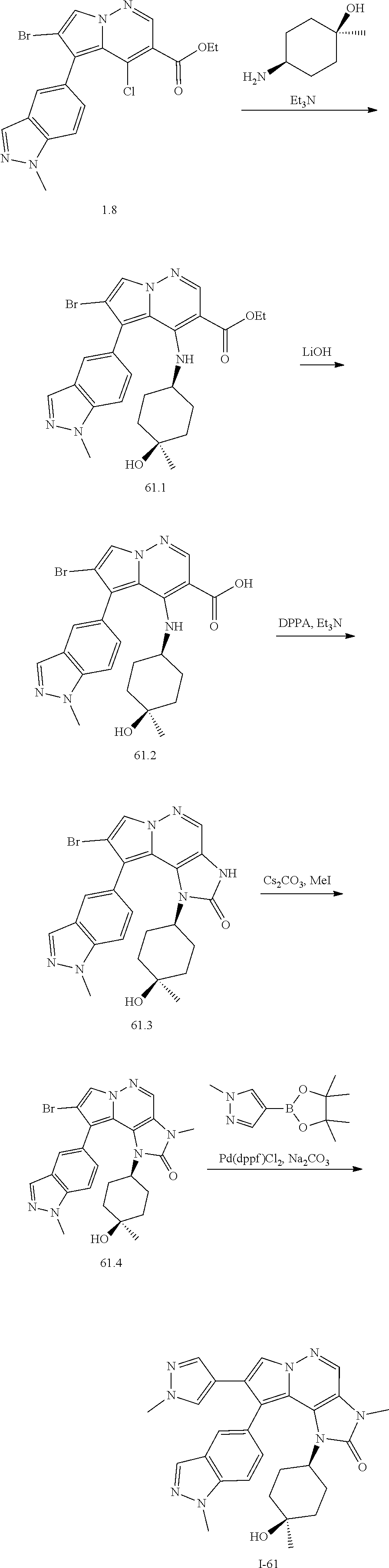

Synthesis of compound 61.1. Compound 61.1 was prepared from compound 1.8 and (1s,4s)-4-amino-1-methylcyclohexan-1-ol following the procedure described in the synthesis of compound 1.9. The product was purified by flash column chromatography on silica gel (Combiflash®, 50% ethyl acetate in hexane). MS(ES): m/z 526.4 and 528.3 [M+H]⁺.

Synthesis of compound 61.2. Compound 61.2 was prepared from compound 61.1 following the procedure described in the synthesis of compound 1.10. The product was triturated with pentane, dried under vacuum and used in the next step without purification. MS(ES): m/z 498.2 and 500.2 [M+H]⁺.

Synthesis of compound 61.3. Compound 61.3 was prepared from compound 61.2 following the procedure described in the synthesis of compound 1.11. The product was purified by flash column chromatography on silica gel (Combiflash®, 1.8% methanol in DCM). MS(ES): m/z 495.3 & 497.3 [M+H]⁺.

Synthesis of compound 61.4. Compound 61.4 was prepared from compound 61.3 following the procedure described in the synthesis of compound 1.12. The product was purified by flash column chromatography on silica gel (1.4% methanol in DCM). MS(ES): m/z 509.1 and 511.1 [M+H]⁺.

Synthesis of compound I-61. Compound I-61 was prepared from compound 61.4 following the procedure described in the synthesis of compound 1.13. The product was purified by flash column chromatography on silica gel (Combiflash®, 0-3.2% methanol in DCM). MS(ES): m/z 511.3 [M+H]⁺, ¹H-NMR (400 MHz, DMSO-d₆): δ 8.38 (s, 1H), 8.14-8.09 (m, 2H), 7.79 (d, J=8.4 Hz, 1H), 7.74 (s, 1H), 7.36 (dd, J=1.6 Hz, 8.4 Hz, 1H), 7.19 (s, 1H), 6.86 (s, 1H), 4.11 (s, 3H), 3.64 (s, 3H), 3.39 (s, 3H), 3.27-3.12 (m, 1H), 2.40-2.30 (m, 2H), 1.25-1.23 (m, 1H), 1.18-1.10 (m, 1H), 0.93-1.02 (m, 3H), 0.54 (s, 3H), 0.10-0.07 (m, 1H), −0.43-−0.47 (m, 1H).

### Example I-62: (1R,2R)-2-fluoro-N-((1s,4S)-4-(9-(4-methoxyphenyl)-3-methyl-8-(1-methyl-1H-pyrazol-4-yl)-2-oxo-2,3-dihydro-1H-imidazo[4,5-d]pyrrolo[1,2-b]pyridazin-1-yl)-1-methylcyclohexyl)cyclopropane-1-carboxamide

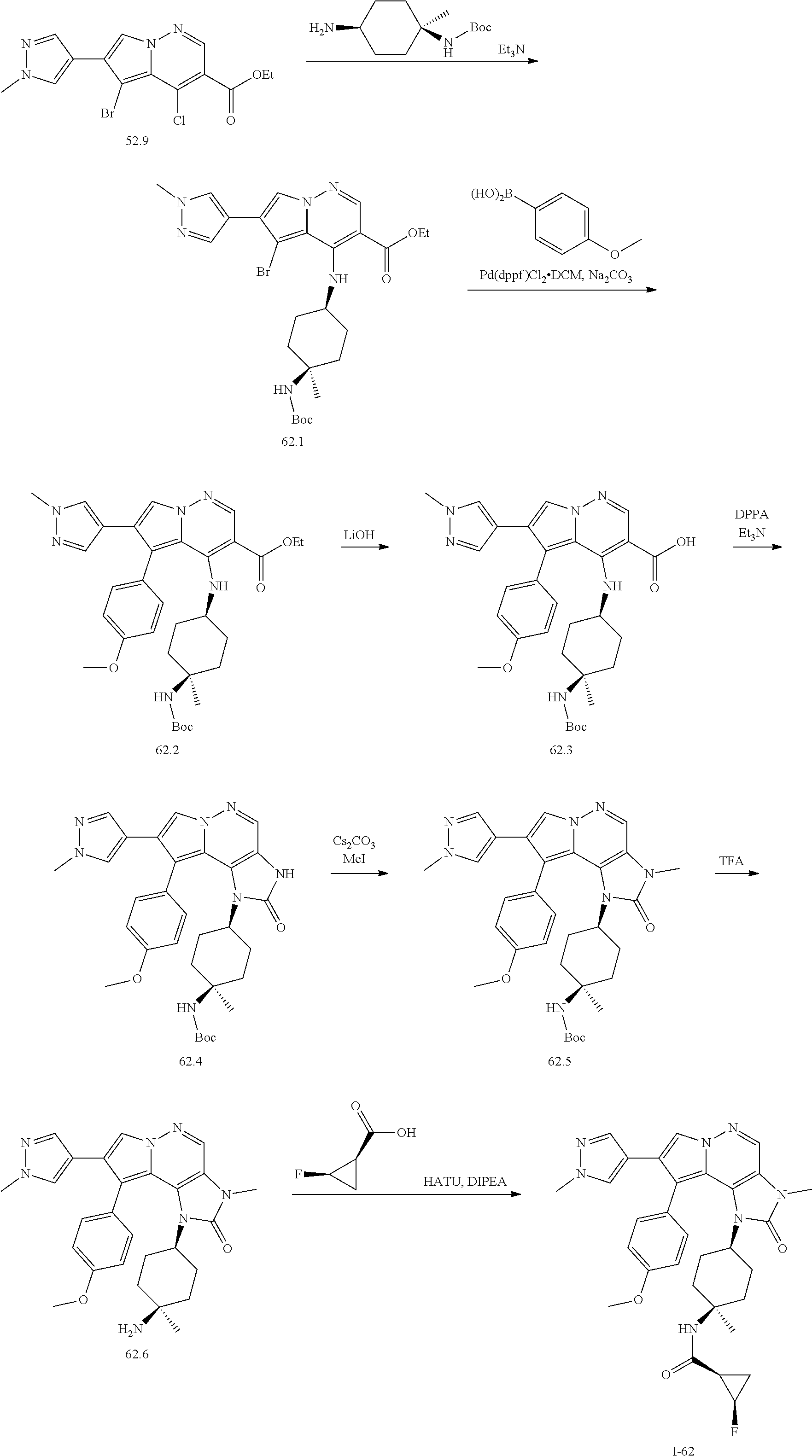

Synthesis of compound 62.1. Compound 62.1 was prepared from compound 52.9 and tert-butyl ((1s,4s)-4-amino-1-methylcyclohexyl)carbamate following the procedure described in the synthesis of compound 1.9. The product was purified by flash column chromatography on silica gel (30% ethyl acetate in hexane). MS (ES): 576.60 [M+H]⁺.

Synthesis of compound 62.2. Compound 62.2 was prepared from compound 62.1 and (4-methoxyphenyl)boronic acid following the procedure described in the synthesis of compound 1.13. The product was purified by flash column chromatography on silica gel (16% ethyl acetate in n-hexane). MS (ES): m/z 603.5 [M+H]⁺.

Synthesis of compound 62.3. Compound 62.3 was prepared from compound 62.2 following the procedure described in the synthesis of compound 1.10. The product was used in the next step without purification. MS (ES): m/z 575.6 [M+H]⁺.

Synthesis of compound 62.4. Compound 62.4 was prepared from compound 62.3 following the procedure described in the synthesis of compound 1.11. The product was purified by flash column chromatography on silica gel (2.1% methanol in DCM). MS(ES): m/z 572.6 [M+H]⁺.

Synthesis of compound 62.5. Compound 62.5 was prepared from compound 62.4 following the procedure described in the synthesis of compound 1.12. The product was purified by flash column chromatography on silica gel (2.5% methanol in DCM). MS(ES): m/z 586.7 [M+H]⁺.

Synthesis of compound 62.6. Compound 62.6 was prepared from compound 62.5 following the procedure described in the synthesis of compound 1.14. The product was directly used in the next step without purification. MS (ES): m/z 484.6 [M+H]⁺.

Synthesis of compound I-62. Compound I-62 was prepared from compound 62.6 and (1R,2R)-2-fluorocyclopropane-1-carboxylic acid following the procedure described in the synthesis of compound I-45. The product was purified by flash column chromatography on silica gel (3.5% methanol in DCM). MS (ES): m/z 572.3 [M+H]⁺, ¹H NMR (DMSO-d₆, 400 MHz): δ 8.36 (s, 1H), 8.11 (s, 1H), 7.40 (s, 1H), 7.28 (d, J=3.2 Hz, 2H), 7.17 (d, J=2.8 Hz, 1H), 7.11 (d, J=2.8 Hz, 2H), 6.98 (d, J=2.8 Hz, 1H), 4.75-4.60 (m, 1H), 3.86 (s, 3H), 3.70 (s, 3H), 3.40 (s, 3H), 2.40 (s, 1H), 2.08 (d, J=12.0 Hz, 1H), 1.98 (d, J=5.6 Hz, 1H), 1.83 (t, J=5.2 Hz, 1H), 1.45 (d, J=3.6 Hz, 1H), 1.40 (s, 1H), 1.29 (d, J=2.8 Hz, 3H), 1.24 (d, J=9.6 Hz, 5H), 0.95-0.90 (m, 1H), 0.91 (t, J=3.6 Hz, 1H), 0.42 (t, J=13.6 Hz, 2H).

### Example I-63: (1R,2R)—N-((1s,4S)-4-(9-(benzo[d][1,3]dioxol-5-yl)-3-methyl-8-(1-methyl-1H-pyrazol-4-yl)-2-oxo-2,3-dihydro-1H-imidazo[4,5-d]pyrrolo[1,2-b]pyridazin-1-yl)-1-methylcyclohexyl)-2-fluorocyclopropane-1-carboxamide

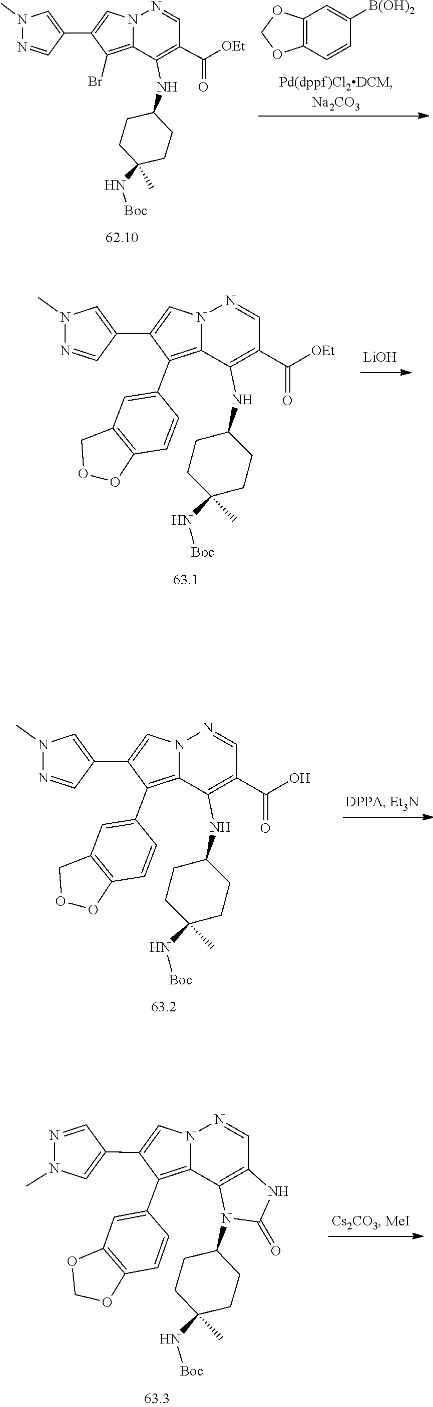

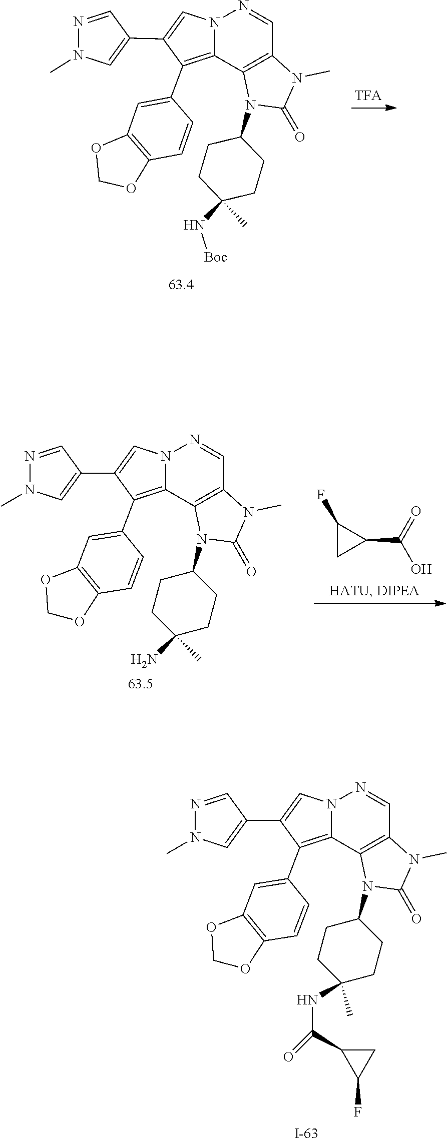

Synthesis of compound 63.1. Compound 63.1 was prepared from compound 62.1 and benzo[d][1,3]dioxol-5-ylboronic acid following the procedure described in the synthesis of compound 1.13. The product was purified by flash column chromatography on silica gel (16% ethyl acetate in n-hexane). MS (ES): m/z 617.7 [M+H]⁺.

Synthesis of compound 63.2. Compound 63.2 was prepared from compound 63.1 following the procedure described in the synthesis of compound 1.10. The product was used in the next step without purification. MS (ES): m/z 589.6 [M+H]⁺.

Synthesis of compound 63.3. Compound 63.3 was prepared from compound 63.2 following the procedure described in the synthesis of compound 1.11. The product was purified by flash column chromatography on silica gel (2.3% methanol in DCM). MS(ES): m/z 586.6 [M+H]⁺.

Synthesis of compound 63.4. Compound 63.4 was prepared from compound 63.3 following the procedure described in the synthesis of compound 1.12. The product was purified by flash column chromatography on silica gel (2.4% methanol in DCM). MS(ES): m/z 600.9 [M+H]⁺.

Synthesis of compound 63.5. Compound 63.5 was prepared from compound 63.4 following the procedure described in the synthesis of compound 1.14. The product was directly used in the next step without purification. MS (ES): m/z 500.5 [M+H]⁺.

Synthesis of compound I-63. Compound I-63 was prepared from compound 63.5 and (1R,2R)-2-fluorocyclopropane-1-carboxylic acid following the procedure described in the synthesis of compound I-45. The product was purified by flash column chromatography on silica gel (3.5% methanol in DCM). MS (ES): m/z 586.3 [M+H]⁺, ¹H NMR (DMSO-d₆, 400 MHz): δ 8.36 (s, 1H), 8.10 (s, 1H), 7.45 (s, 1H), 7.23 (s, 1H), 7.08 (s, 2H), 6.96 (d, J=14.8 Hz, 1H), 6.84 (d, J=7.6 Hz, 1H), 6.12 (d, J=5.2 Hz, 2H), 4.81-4.65 (m, 1H), 3.61 (m, 1H), 3.47 (m, 1H), 3.14 (m, 1H), 2.32-1.99 (m, 2H), 1.84 (m, 1H), 1.54-1.46 (m, 1H), 1.26 (s, 6H), 1.11 (m, 5H), 0.94 (m, 1H), 0.49-0.52 (m, 2H).

### Example I-64: methyl ((1R,3R)-3-(3-methyl-8-(1-methyl-1H-pyrazol-4-yl)-2-oxo-9-(thieno[2,3-b]pyridin-2-yl)-2,3-dihydro-1H-imidazo[4,5-d]pyrrolo[1,2-b]pyridazin-1-yl)cyclopentyl)carbamate

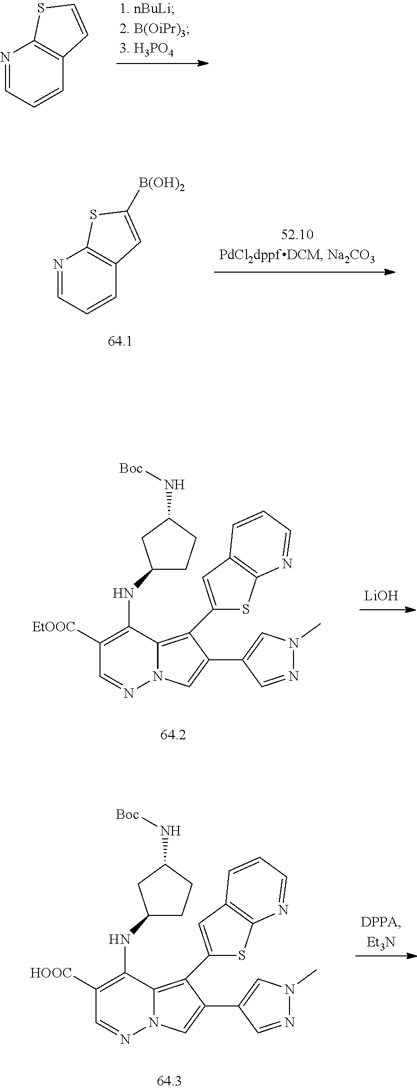

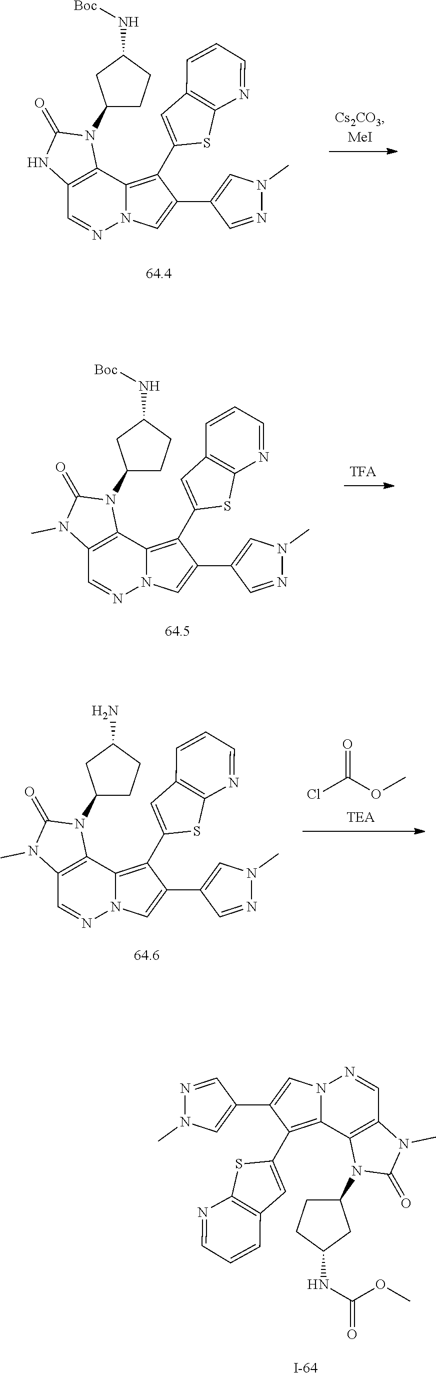

Synthesis of compound 64.1. To a solution of thieno[2,3-b]pyridine (1.0 g, 7.4 mmol, 1.0 equiv) in THF (20 mL) at −78° C. was added n-butyl lithium (2.5M) (3.2 mL, 8.14 mmol, 1.1 equiv) dropwise over 30 min and stirred for 1 h. To the solution was added triisopropylborate (1.70 g, 8.88 mmol, 1.2 equiv) at −78° C. and the mixture was stirred at room temperature for 2 h. Orthophosphoric acid (1 mL) was added and stirred for 30 min. The mixture was transferred into ice-water and extracted with DCM. The combined organic layers were washed with brine, dried over anhydrous sodium sulfate, filtered and concentrated under reduced pressure to afford material 64.1 (0.500 g, 38%). MS(ES): m/z 180.1 [M+H]⁺.

Synthesis of compound 64.2. Compound 64.2 was prepared from compound 64.1 and 52.10 following the procedure described in the synthesis of compound 1.13. The product was purified by flash column chromatography on silica gel (40% ethyl acetate in hexane). MS(ES): m/z 602.26 [M+H]⁺.

Synthesis of compound 64.3. Compound 64.3 was prepared from compound 64.2 following the procedure described in the synthesis of compound 1.10. The product was triturated with pentane, dried under vacuum and used in the next step without purification. MS(ES): m/z 574.24 [M+H]⁺.

Synthesis of compound 64.4. Compound 64.4 was prepared from compound 64.3 following the procedure described in the synthesis of compound 1.11. The product was purified by flash column chromatography on silica gel (Combiflash®, 1.6% methanol in DCM). MS(ES): m/z 571.17 [M+H]⁺.

Synthesis of compound 64.5. Compound 64.5 was prepared from compound 64.4 following the procedure described in the synthesis of compound 1.12. The product was purified by flash column chromatography on silica gel (1.2% methanol in DCM). MS(ES): m/z 585.25 [M+H]⁺.

Synthesis of compound 64.6. Compound 64.6 was prepared from compound 64.5 following the procedure described in the synthesis of compound 1.14. The product was triturated with pentane and dried under reduced pressure. It was used in the next step without purification. MS(ES): m/z 485.18 [M+H]⁺.

Synthesis of I-64. Compound I-64 was prepared from compound 64.6 following the procedure described in the synthesis of compound I-1. The product was purified by flash column chromatography on silica gel (Combiflash®, 3.8% methanol in DCM). MS(ES): m/z 543.37 [M+H]⁺, ¹H-NMR (400 MHz, DMSO-d₆): δ 8.64 (d, J=3.6 Hz, 1H), 8.51 (s, 1H), 8.31 (d, J=8.0 Hz, 1H), 8.21 (s, 1H), 7.60 (s, 1H), 7.55 (s, 1H), 7.54-7.51 (m, 1H), 7.30 (s, 1H), 6.61 (s, 1H), 4.05-3.93 (m, 2H), 3.73 (s, 3H), 3.46 (s, 3H), 3.39 (s, 3H), 2.13-2.09 (m, 1H), 2.08-1.96 (m, 1H), 1.65-1.52 (m, 1H), 1.48-1.41 (m, 1H), 1.30-1.22 (m, 1H), 0.01 (m, 1H).

### Example I-65: methyl ((1r,3r)-3-(3-methyl-8-(1-methyl-1H-pyrazol-4-yl)-9-(4-methyl-3,4-dihydro-2H-benzo[b][1,4]oxazin-7-yl)-2-oxo-2,3-dihydro-1H-imidazo[4,5-d]pyrrolo[1,2-b]pyridazin-1-yl)cyclobutyl)carbamate

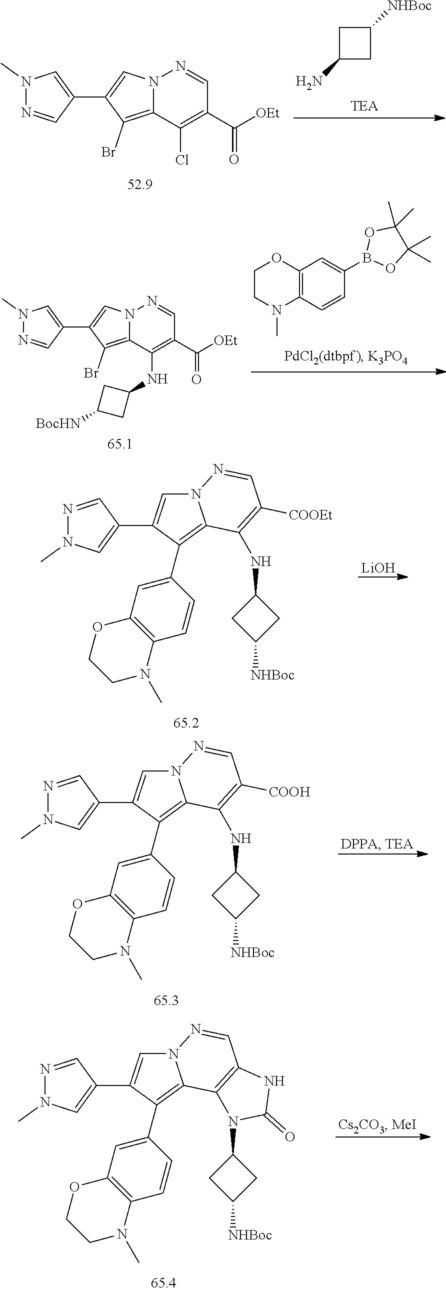

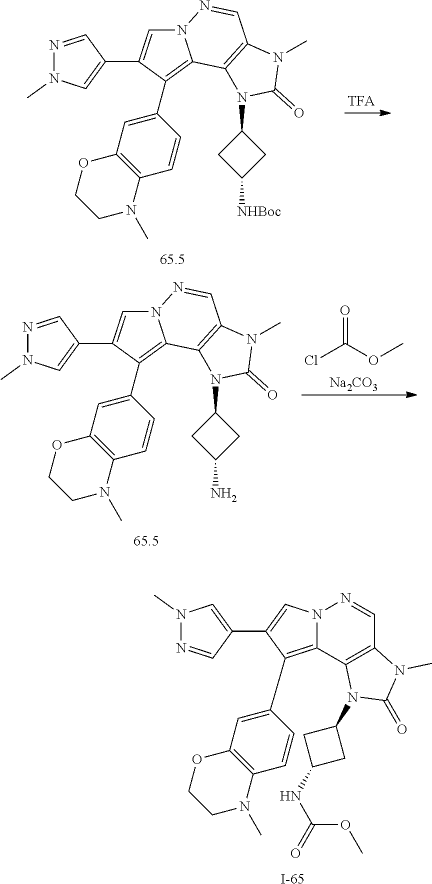

Synthesis of compound 65.1. Compound 65.1 was prepared from compound 52.9 and tert-butyl ((1r,3r)-3-aminocyclobutyl)carbamate following the procedure described in the synthesis of compound 1.9. The product was purified by flash column chromatography on silica gel (30% ethyl acetate in hexane). MS(ES): m/z 533.3 and 535.3 [M+H]⁺.

Synthesis of compound 65.2. Compound 65.2 was prepared from compound 65.1 and 4-methyl-7-(4,4,5,5-tetramethyl-1,3,2-dioxaborolan-2-yl)-3,4-dihydro-2H-benzo[b][1,4]oxazine following the procedure described in the synthesis of compound 59.12. The product was purified by flash column chromatography on silica gel (40% ethyl acetate in hexane). MS(ES): m/z 602 [M+H]⁺.

Synthesis of compound 65.3. Compound 65.3 was prepared from compound 65.2 following the procedure described in the synthesis of compound 1.10. The product was used in the next step without purification. MS(ES): m/z 574.4 [M+H]⁺.

Synthesis of compound 65.4. Compound 65.4 was prepared from compound 65.3 following the procedure described in the synthesis of compound 1.11. The product was purified by flash column chromatography on silica gel (Combiflash®, 2.1% methanol in DCM). MS(ES): m/z 571.3 [M+H]⁺.

Synthesis of compound 65.5. Compound 65.5 was prepared from compound 65.4 following the procedure described in the synthesis of compound 1.12. The product was purified by flash column chromatography on silica gel (1.6% methanol in DCM). MS(ES): m/z 585.3 [M+H]⁺.

Synthesis of compound 65.6. Compound 65.6 was prepared from compound 65.5 following the procedure described in the synthesis of compound 1.14. The product was used in next step without purification. MS(ES): m/z 485.3 [M+H]⁺.

Synthesis of I-65. Compound I-65 was prepared from compound 65.6 following the procedure described in the synthesis of compound I-1. The product was purified by flash column chromatography on silica gel (Combiflash®, 4.2% methanol in DCM). MS(ES): m/z 542.9 [M+H]⁺, HPLC purity: 95%, ¹H-NMR (400 MHz, DMSO-d₆): δ 8.35 (d, J=2.4 Hz, 1H), 8.04 (d, J=2.4 Hz, 1H), 7.39 (s, 1H), 7.11 (s, 1H), 6.81 (d, J=8.4 Hz, 1H), 6.75 (d, J=8.0 Hz, 2H), 6.54 (d, J=1.6 Hz, 1H), 4.28 (s, 2H), 4.19-4.18 (m, 1H), 4.00 (d, J=4.8 Hz, 1H), 3.74 (s, 3H), 3.52 (s, 3H), 3.36 (s, 3H), 3.34 (s, 2H), 2.91 (m, 3H), 2.89-2.85 (m, 2H), 1.84-1.75 (m, 2H).

### Example I-66: 1-((1s,4s)-4-hydroxy-4-methylcyclohexyl)-3-methyl-8-(1-methyl-1H-1,2,3-triazol-4-yl)-9-(1-methyl-1H-indazol-5-yl)-1,3-dihydro-2H-imidazo[4,5-d]pyrrolo[1,2-b]pyridazin-2-one

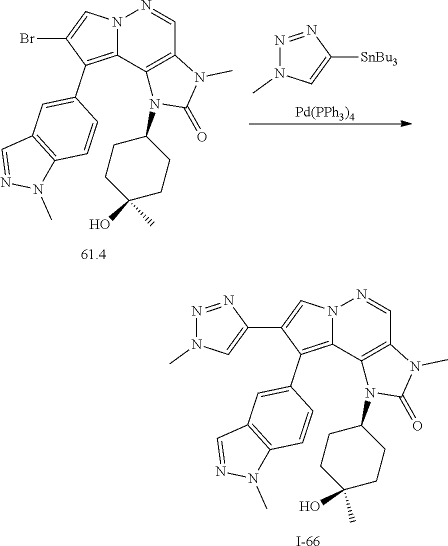

Synthesis of compound I-66. A mixture of 61.4 (0.200 g, 0.39 mmol, 1.0 equiv) and 1-methyl-4-(tributylstannyl)-1H-1,2,3-triazole (0.587 g, 1.57 mmol, 4.0 equiv) in 1,4-dioxane (10 mL) was degassed by bubbling through a stream of argon for 10 min. Pd(PPh₃)₄ (0.045 g, 0.039 mmol, 0.1 equiv) was added to the reaction mixture and purged with argon for 10 min. The mixture was stirred at 140° C. for 3 h. It was poured into water and extracted with ethyl acetate. The combined organic layers were washed with brine, dried over anhydrous sodium sulfate, filtered and concentrated under reduced pressure. The residue was purified by flash column chromatography on silica gel (Combiflash®, 3.2% methanol in DCM) to afford I-66 (0.023 g, 11.5%). MS(ES): m/z 512.3 [M+H]⁺, ¹H-NMR (400 MHz, DMSO-d₆): δ 8.56 (s, 1H), 8.46 (s, 1H), 8.06 (s, 1H), 7.73-7.71 (m, 2H), 7.39-7.33 (m, 1H), 6.92 (s, 1H), 4.08 (s, 3H), 3.98 (s, 3H), 3.72 (d, J=4.4 Hz, 1H), 3.38 (s, 3H), 3.20-3.10 (m, 1H), 2.46-2.36 (m, 2H), 1.16-1.13 (m, 2H), 0.99-0.92 (m, 4H), 0.52 (s, 3H).

### Example I-67: methyl ((1r,3r)-3-(3-methyl-8-(1-methyl-1H-pyrazol-4-yl)-9-(7-methyl-2,3-dihydrothieno[3,4-b][1,4]dioxin-5-yl)-2-oxo-2,3-dihydro-1H-imidazo[4,5-d]pyrrolo[1,2-b]pyridazin-1-yl)cyclobutyl)carbamate

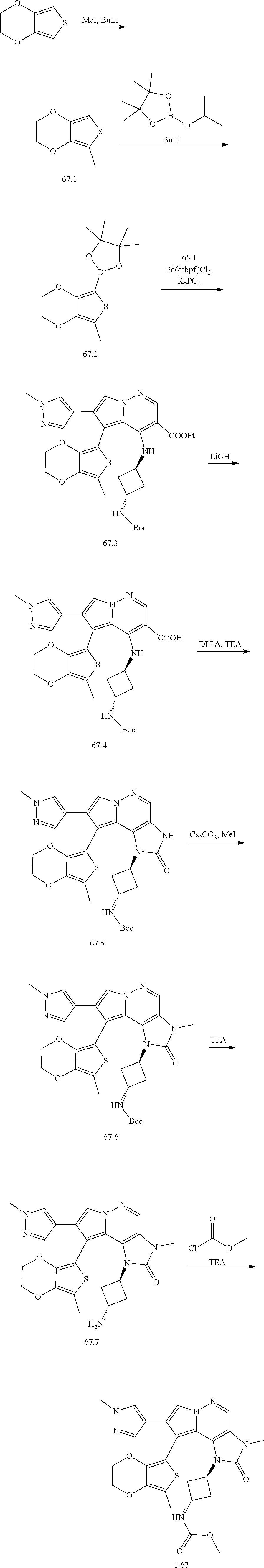

Synthesis of compound 67.1. To a stirred solution of 2,3-dihydrothieno[3,4-b][1,4]dioxine (2.00 g, 14.1 mmol, 1 equiv) in THF (30 mL) was added n-BuLi (2.5 M in hexane, 5.6 mL, 14.1 mmol, 1 equiv) at −78° C. The reaction mixture was stirred at same temperature for 1 h. Methyl iodide (2.2 g, 15.4 mmol, 1.1 equiv) was added and stirred for 1 h. It was transferred into ice-water and quenched with a saturated aqueous solution of NH₄Cl and extracted with ethyl acetate. The combined organic layers were washed with brine, dried over anhydrous sodium sulfate, filtered and concentrated under reduced pressure. The residue was triturated with n-pentane to afford 67.1 (1.2 g, 55%).

Synthesis of compound 67.2. To a stirred solution of 67.1 (1.2 g, 7.6 mmol, 1 equiv) in (30 mL) of THF was added n-BuLi (2.5 M in hexane, 5.6 mL, 14.1 mmol, 1 equiv) at −78° C. and stirred for 1 h. To the solution was added 2-isopropoxy-4,4,5,5-tetramethyl-1,3,2-dioxaborolane (4.29 g, 23.5 mmol, 3 equiv). The reaction mixture allowed to warm to room temperature and stirred for 16 h. It was transferred into ice-water and quenched by a saturated aqueous solution of NH₄Cl and extracted with ethyl acetate. The combined organic layers were washed with brine, dried over anhydrous sodium sulfate, filtered and concentrated under reduced pressure. The residue was triturated with n-pentane to afford 67.2 (0.6 g, 28%).

Synthesis of compound 67.3. Compound 67.3 was prepared from compound 65.1 and 67.2 following the procedure described in the synthesis of compound 59.12. The product was purified by flash column chromatography on silica gel (2.1% methanol in DCM). MS (ES): m/z 609.9 [M+H]⁺.

Synthesis of compound 67.4. Compound 67.4 was prepared from compound 67.3 following the procedure described in the synthesis of compound 1.10. The product was triturated with n-pentane, dried under vacuum and used in the next step without purification. MS (ES): m/z 581.8 [M+H]⁺.

Synthesis of compound 67.5. Compound 67.5 was prepared from compound 67.4 following the procedure described in the synthesis of compound 1.11. The product was purified by flash column chromatography on silica gel (Combiflash®, 2.4% methanol in DCM). MS (ES): m/z 578.58 [M+H]⁺.

Synthesis of compound 67.6. Compound 67.6 was prepared from compound 67.5 following the procedure described in the synthesis of compound 1.12. The product was purified by flash column chromatography on silica gel (2.5% methanol in DCM). MS (ES): m/z 592.92 [M+H]⁺.

Synthesis of compound 67.7. Compound 67.7 was prepared from compound 67.6 following the procedure described in the synthesis of compound 1.14. The product was used in the next step without purification. MS (ES): m/z 492.52 [M+H]⁺.

Synthesis of compound I-67. Compound I-67 was prepared from compound 67.6 following the procedure described in the synthesis of compound I-1. The product was purified by flash column chromatography on silica gel (Combiflash®, 3.7% methanol in DCM). MS (ES): m/z 550.2 [M+H]⁺, ¹H NMR (400 MHz, DMSO-d₆): δ 8.43 (s, 1H), 8.12 (s, 1H), 7.50 (s, 1H), 7.30 (s, 1H), 7.06 (s, 1H), 4.62 (m, 1H), 4.22 (m, 2H), 4.13 (m, 3H), 3.80 (s, 3H), 3.60 (s, 3H), 3.39 (s, 3H), 3.01-3.02 (m, 2H), 2.28-2.28 (m, 2H), 2.05-2.09 (m, 3H).

### Example I-68: methyl ((1R,3R)-3-(8-(3,6-dihydro-2H-pyran-4-yl)-3-methyl-2-oxo-9-(5-(trifluoromethyl)-1H-pyrazol-3-yl)-2,3-dihydro-1H-imidazo[4,5-d]pyrrolo[1,2-b]pyridazin-1-yl)cyclopentyl)carbamate

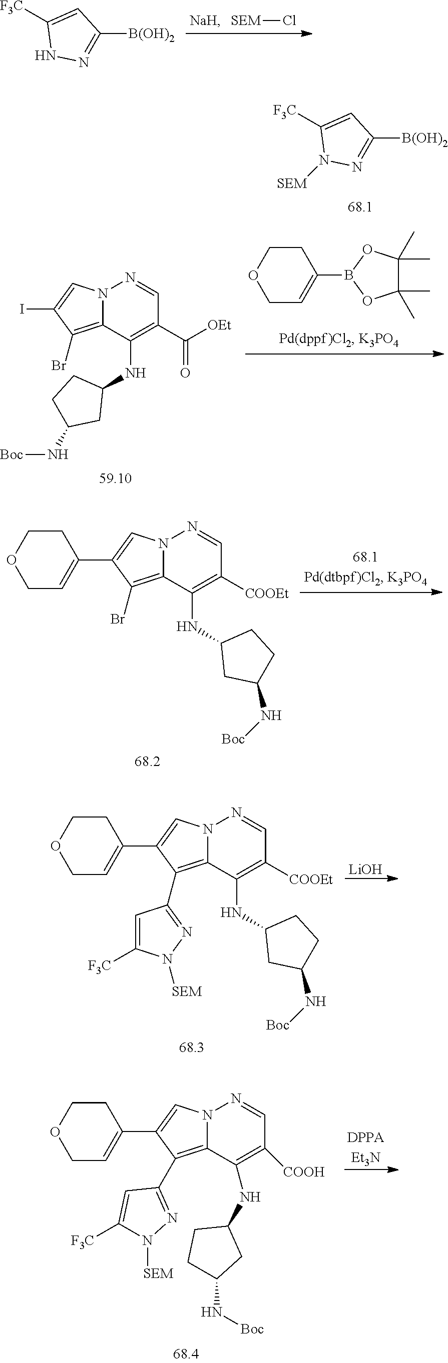

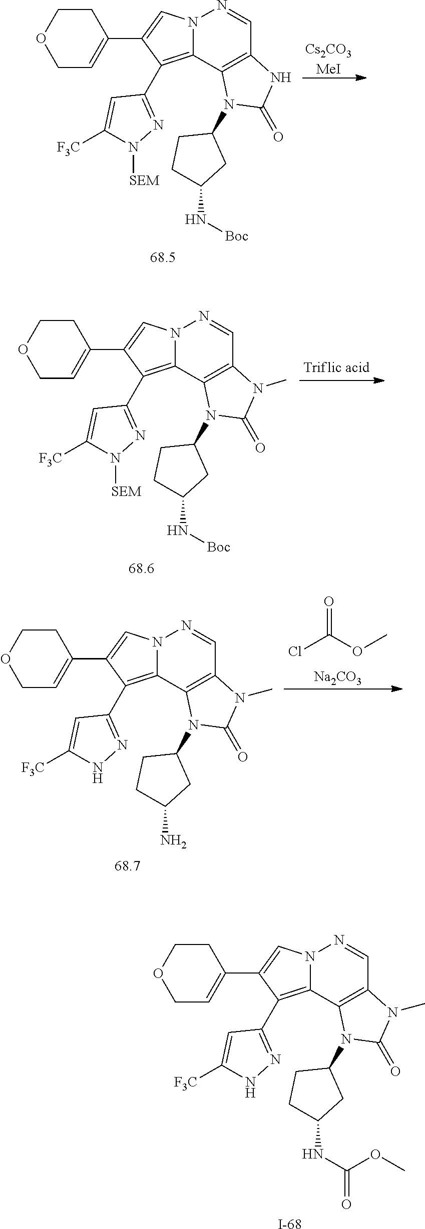

Synthesis of compound 68.1. To a solution of (5-(trifluoromethyl)-1H-pyrazol-3-yl)boronic acid (1.0 g, 0.555 mmol, 1.0 equiv) in THF (3.0 mL) at 0° C. was added sodium hydride (60% dispersion in mineral oil, 0.444 g, 1.111 mmol, 2.0 equiv) and 2-(trimethylsilyl) ethoxy methyl chloride (1.38 g, 0.832 mmol, 1.5 equiv) was added at room temperature. The reaction mixture was stirred for 1 h. It was transferred into ice-water and extracted with ethyl acetate. The combined organic layers were washed with brine, dried over anhydrous sodium sulfate, filtered and concentrated under reduced pressure to afford 68.1 (0.80 g, 46.5%). MS(ES): m/z 311.3 [M+H]⁺. The residue was used in the next step without purification.

Synthesis of compound 68.2. Compound 68.2 was prepared from compound 59.10 and 2-(3,6-dihydro-2H-pyran-4-yl)-4,4,5,5-tetramethyl-1,3,2-dioxaborolane following the procedure described in the synthesis of compound 54.3. The product was purified by flash column chromatography on silica gel (50% ethyl acetate in hexane). MS(ES): m/z 549.34 and 551.34 [M+H]⁺.

Synthesis of compound 68.3. Compound 68.2 was prepared from compound 68.1 and 68.2 following the procedure described in the synthesis of compound 59.12. The product was purified by flash column chromatography on silica gel (50% ethyl acetate in hexane). MS(ES): m/z 735.3 [M+H]⁺.

Synthesis of compound 68.4. Compound 68.4 was prepared from compound 68.3 following the procedure described in the synthesis of compound 1.10. The product was triturated with pentane, dried under vacuum and used in the next step without purification. MS(ES): m/z 707.31 [M+H]⁺.

Synthesis of compound 68.5. Compound 68.5 was prepared from compound 68.4 following the procedure described in the synthesis of compound 1.11. The product was purified by flash column chromatography on silica gel (Combiflash®, 1.8% methanol in DCM). MS(ES): m/z 704.5 [M+H]⁺.

Synthesis of compound 68.6. Compound 68.6 was prepared from compound 68.5 following the procedure described in the synthesis of compound 1.12. The product was purified by flash column chromatography on silica gel (1.4% methanol in DCM. MS(ES): m/z 718.33 [M+H]⁺.

Synthesis of compound 68.7. Compound 68.7 was prepared from compound 68.6 following the procedure described in the synthesis of compound 1.14. The product was used in the next step without purification. MS(ES): m/z 488.22 [M+H]⁺.

Synthesis of I-68. Compound I-68 was prepared from compound 68.7 following the procedure described in the synthesis of compound I-1. The product was purified by flash column chromatography on silica gel (Combiflash®, 4.0% methanol in DCM). MS(ES): m/z 546.3 [M+H]⁺, ¹H-NMR (400 MHz, DMSO-d₆): δ 13.98 (s, 1H), 8.54 (s, 1H), 8.07 (s, 1H), 7.02 (s, 1H), 6.92 (s, 1H), 5.39 (s, 1H), 4.12 (s, 1H), 4.00 (s, 2H), 3.71 (t, J=5.2 Hz, 2H), 3.48 (s, 3H), 3.38 (s, 3H), 2.24 (s, 2H), 2.09 (d, J=4.0 Hz, 1H), 1.92-1.97 (m, 2H), 1.54 (t, J=7.2 Hz, 1H), 1.32 (s, 1H), 0.92 (t, J=7.2 Hz, 1H).

### Example I-69: methyl ((1R,3R)-3-(8-(3,6-dihydro-2H-pyran-4-yl)-9-(4-fluoro-1-methyl-2-oxo-2,3-dihydro-1H-benzol[d]imidazol-5-yl)-3-methyl-2-oxo-2,3-dihydro-1H-imidazo[4,5-d]pyrrolo[1,2-b]pyridazin-1-yl)cyclopentyl)carbamate

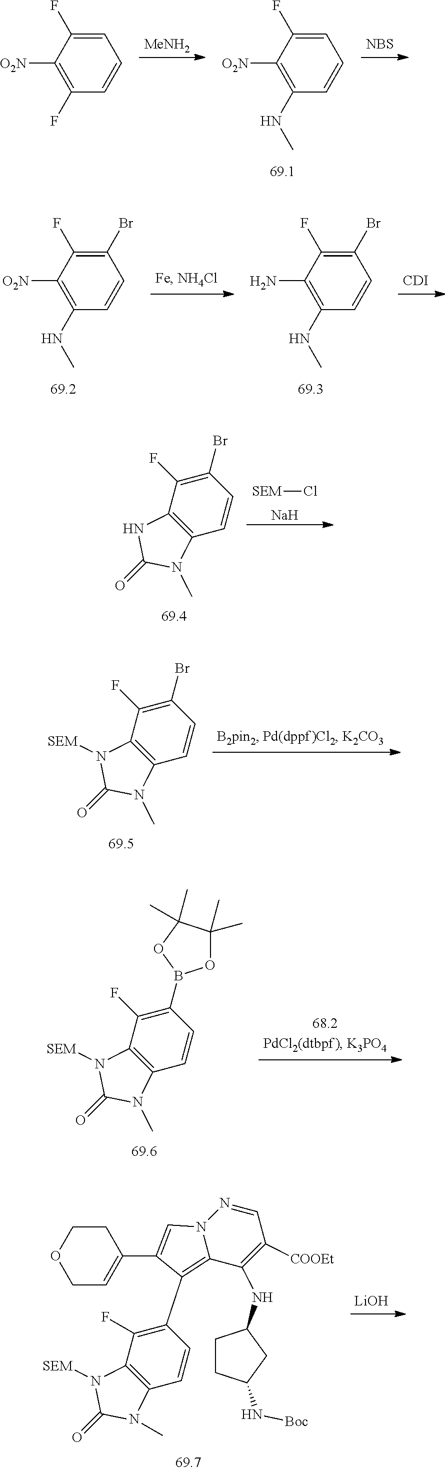

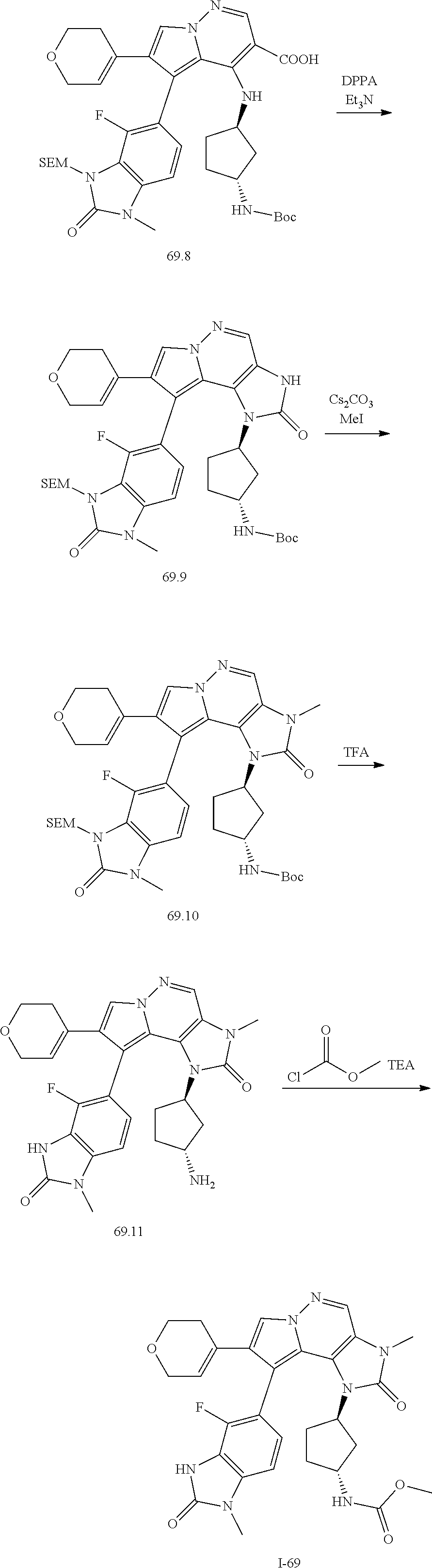

Synthesis of compound 69.1. To a solution of 1,3-difluoro-2-nitrobenzene (10 g, 62.86 mmol, 1.0 equiv) in THF (100 mL) was added methylamine (2.0 M in THF, 38 mL, 75.42 mmol, 1.2 equiv) at 0° C. The reaction mixture was stirred at room temperature for 1 h. It was transferred ice-water and extracted with ethyl acetate. The combined organic layers were washed with brine, dried over anhydrous sodium sulfate, filtered and concentrated under reduced pressure. The residue was purified by flash column chromatography on silica gel (25% ethyl acetate in hexane to afford 69.1 (8.2 g, 77%). MS(ES): m/z 171.3 [M+H]⁺.

Synthesis of compound 69.2. To a solution of 69.1 (8.2 g, 48.19 mmol, 1.0 equiv) in DMF (82 mL) was added N-bromosuccinimide (9.43 g, 53.0 mmol, 1.1 equiv) in small portions at 0° C. The reaction mixture was stirred at rt for 1 h. It was transferred into ice-water and extracted with ethyl acetate. The combined organic layers were washed with brine, dried over anhydrous sodium sulfate, filtered and concentrated under reduced pressure to afford product. The residue was purified by flash column chromatography on silica gel (35% ethyl acetate in hexane) to afford 69.2 (6.8 g, 57%). MS(ES): m/z 249 and 251 [M+H]⁺.

Synthesis of compound 69.3. A mixture of 69.2 (6.8 g, 27.30 mmol, 1.0 equiv), iron powder (15.23 mg, 273.0 mmol, 10 equiv), methanol (70 mL) and a saturated aqueous solution of ammonium chloride (10 mL) was stirred at 80° C. for 2 h. It was diluted with methanol and filtered on a Celite bed and the filtrate was concentrated under reduced pressure. The residue was purified by flash column chromatography on silica gel (Combiflash®, 45% ethyl acetate in hexane) to afford 69.3 (4.8 g, 80%). MS(ES): m/z 219.3 and 221.2 [M+H]⁺.

Synthesis of compound 69.4. To a solution of 69.3 (4.8 g, 21.91 mmol, 1.0 equiv) in THF (50 mL) was added carbonyl diimidazole (10.66 g, 65.75 mmol, 3.0 equiv) at room temperature and the reaction mixture was stirred at 80° C. for 12 h. It was transferred to ice-water and extracted with ethyl acetate. The combined organic layers were washed with brine, dried over anhydrous sodium sulfate, filtered and concentrated under reduced pressure to afford product. The residue was purified by flash column chromatography on silica gel (Combiflash®, 35% ethyl acetate in hexane) to afford 69.4 (3.3 g, 61%). MS(ES): m/z 245.1 and 247.3 [M+H]⁺.

Synthesis of compound 69.5. To a solution of 69.4 (3.2 g, 13.06 mmol, 1.0 equiv) in DMF (40 mL) was added sodium hydride (1.25 g, 26.12 mmol, 2.0 equiv) at 0° C. and stirred for 30 min. 2-(trimethylsilyl)ethoxy methyl chloride (3.26 g, 19.60 mmol, 1.5 equiv) was added and the reaction mixture was stirred at room temperature for 2 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 15% ethyl acetate in hexane) to afford 69.5 (3.1 g, 63%). MS(ES): m/z 375.4 and 377.5 [M+H]⁺.

Synthesis of compound 69.6. A mixture of 69.5 (1.0 g, 2.66 mmol, 1.0 equiv), bis(pinacolato)diboron (1.01 g, 3.99 mmol, 1.5 equiv) and potassium carbonate (1.10 g, 7.99 mmol, 3.0 equiv) in 1,4-dioxane (10 mL) was purged by bubbling through a stream of argon for 15 min. [1,1′-Bis(diphenylphosphino)ferrocene]dichloropalladium(II) (0.194 g, 0.266 mmol, 0.1 equiv) was added and the reaction mixture was stirred at 100° C. for 2 h. It was cooled to room temperature, transferred into water, and extracted with ethyl acetate. The combined organic layers were washed with brine, dried over anhydrous sodium sulfate, filtered and concentrated under reduced pressure. The residue was purified by flash column chromatography on silica gel (Combiflash®, 22% ethyl acetate in hexane to afford 69.6 (0.390 g, 35%). MS(ES): m/z 423.5 [M+H]⁺.

Synthesis of compound 69.7. Compound 69.7 was prepared from compound 68.2 and 69.6 following the procedure described in the synthesis of compound 59.12. The product was purified by flash column chromatography on silica gel (2.3% methanol in DCM). MS(ES): m/z 766.2 [M+H]⁺.

Synthesis of compound 69.8. Compound 69.8 was prepared from compound 69.7 following the procedure described in the synthesis of compound 1.10. The product was used in the next step without purification. MS(ES): m/z 737.8 [M+H]⁺.

Synthesis of compound 69.9. Compound 69.9 was prepared from compound 69.8 following the procedure described in the synthesis of compound 1.11. The product was purified by flash column chromatography on silica gel (Combiflash®, 2.2% methanol in DCM). MS(ES): m/z 734.9 [M+H]⁺.

Synthesis of compound 69.10. Compound 69.10 was prepared from compound 69.9 following the procedure described in the synthesis of compound 1.12. The product was purified by flash column chromatography on silica gel (1.5% methanol in DCM). MS(ES): m/z 748.8 [M+H]⁺.

Synthesis of compound 69.11. Compound 69.11 was prepared from compound 69.10 following the procedure described in the synthesis of compound 1.14. The product was used in the next step without purification. MS(ES): m/z 518.5 [M+H]⁺.

Synthesis of compound I-69. Compound I-69 was prepared from compound 68.10 following the procedure described in the synthesis of compound I-1. The product was purified by flash column chromatography on silica gel (Combiflash®, 2.3% methanol in DCM). MS (ES): m/z 576.33 [M+H]⁺, ¹H-NMR (400 MHz, DMSO-d₆): δ 11.10 (s, 1H), 8.05 (s, 1H), 7.96 (s, 1H), 7.68-7.63 (m, 2H), 7.28 (d, J=7.6 Hz, 1H), 6.62 (s, 1H), 4.11 (s, 3H), 3.82 (s, 3H), 3.47 (s, 3H), 3.26-3.24 (m, 3H), 3.08-2.98 (m, 2H), 2.15-2.13 (m, 1H), 1.99-1.95 (m, 1H), 1.97-1.74 (m, 2H), 1.31-1.23 (m, 2H), 0.40-0.39 (m, 1H), 0.19-0.18 (m, 1H), −0.11 (m, 1H).

### Example I-70: methyl ((1r,3r)-3-(3-methyl-8-(1-methyl-1H-pyrazol-4-yl)-2-oxo-9-(thieno[2,3-c]pyridin-2-yl)-2,3-dihydro-1H-imidazo[4,5-d]pyrrolo[1,2-b]pyridazin-1-yl)cyclobutyl)carbamate

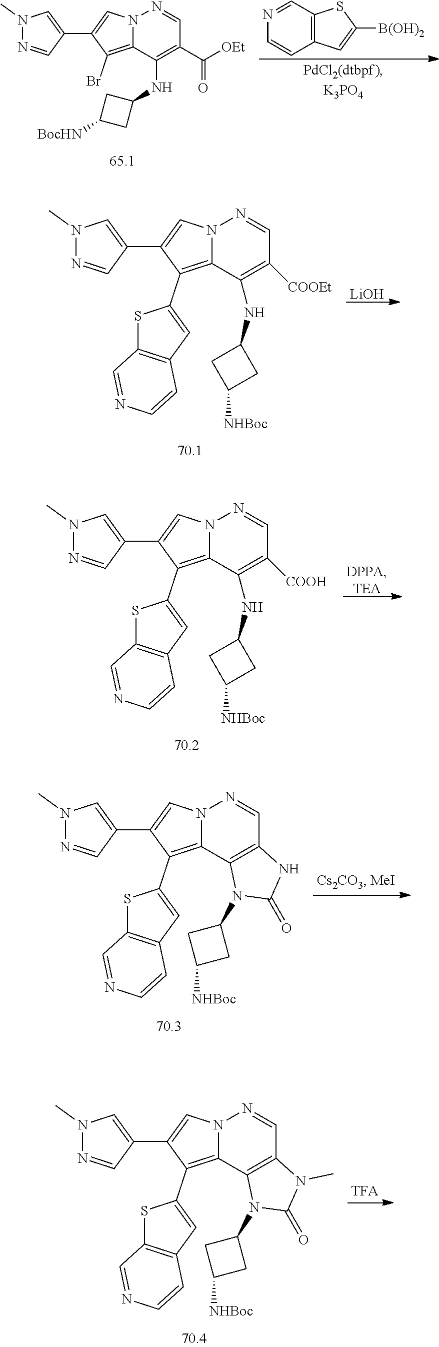

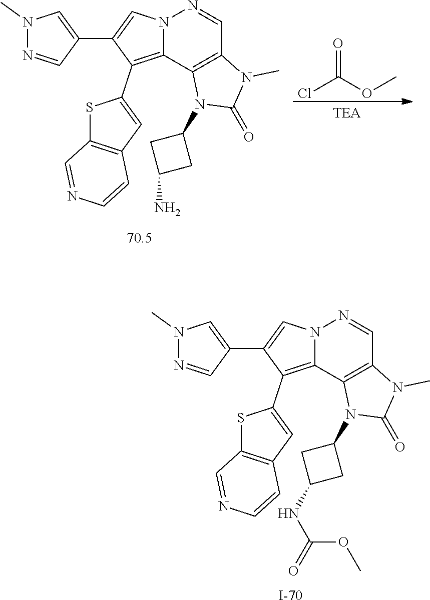

Synthesis of compound 70.1. Compound 70.1 was prepared from compound 65.1 and thieno[2,3-c]pyridin-2-ylboronic acid following the procedure described in the synthesis of compound 54.3. The product was purified by flash column chromatography on silica gel (2.1% methanol in DCM). MS(ES): m/z 588.6 [M+H]⁺.

Synthesis of compound 70.2. Compound 70.2 was prepared from compound 70.1 following the procedure described in the synthesis of compound 1.10. The product was triturated with pentane, dried under vacuum and used in the next step without purification. MS(ES): m/z 560.20 [M+H]⁺.

Synthesis of compound 70.3. Compound 70.3 was prepared from compound 70.2 following the procedure described in the synthesis of compound 1.11. The product was purified by flash column chromatography on silica gel (Combiflash®, 2.4% methanol in DCM). MS(ES): m/z 557.58 [M+H]⁺.

Synthesis of compound 70.4. Compound 70.4 was prepared from compound 70.3 following the procedure described in the synthesis of compound 1.12. The product was purified by flash column chromatography on silica gel (2.1% methanol in DCM). MS(ES): m/z 571.22 [M+H]⁺.

Synthesis of compound 70.5. Compound 70.5 was prepared from compound 70.4 following the procedure described in the synthesis of compound 1.14. The product was triturated with n-pentane and dried under reduced pressure. It was used in the next step without purification. MS(ES): m/z 471.16 [M+H]⁺.

Synthesis of compound I-70. Compound I-70 was prepared from compound 70.5 following the procedure described in the synthesis of compound I-1. The product was purified by flash column chromatography on silica gel (Combiflash®, 3.2% methanol in DCM). MS(ES): m/z 529.2 [M+H]⁺, ¹H-NMR (400 MHz, DMSO-d₆): δ 9.18 (s, 1H), 8.54 (s, 1H), 8.51 (d, J=5.6 Hz, 1H), 8.17 (s, 1H), 7.82 (d, J=5.2 Hz, 1H), 7.58 (s, 1H), 7.53 (s, 1H), 7.21 (s, 1H), 6.64 (s, 1H), 4.25 (m, 1H), 3.83 (m, 1H), 3.72 (s, 3H), 3.40 (s, 3H), 3.38 (s, 3H), 2.84 (m, 2H), 1.61 (m, 2H).

### Example I-71: methyl ((1R,3R)-3-(9-(imidazo[1,5-a]pyridin-7-yl)-8-(3-methoxy-1-methyl-1H-pyrazol-4-yl)-3-methyl-2-oxo-2,3-dihydro-1H-imidazo[4,5-d]pyrrolo[1,2-b]pyridazin-1-yl)cyclopentyl)carbamate

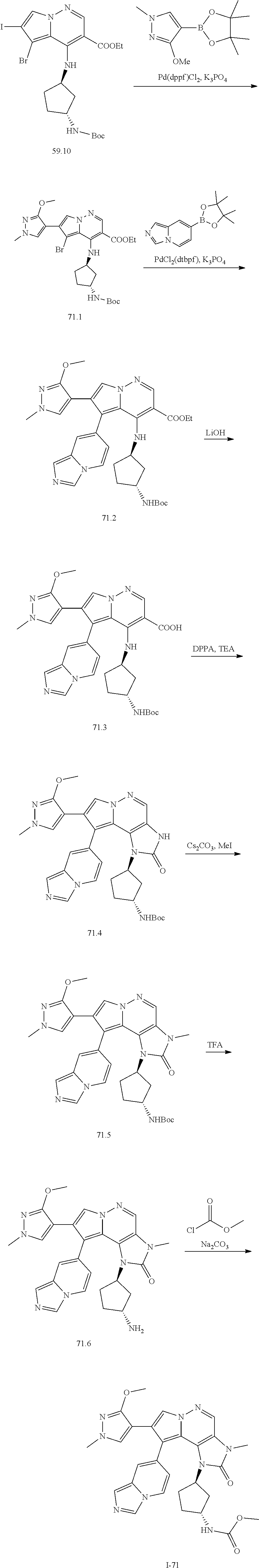

Synthesis of compound 71.1. Compound 71.1 was prepared from compound 59.10 and 3-methoxy-1-methyl-4-(4,4,5,5-tetramethyl-1,3,2-dioxaborolan-2-yl)-1H-pyrazole following the procedure described in the synthesis of compound 54.3. The product was purified by flash column chromatography on silica gel (53% ethyl acetate in hexane). MS(ES): m/z 577.5 and 579.5

Synthesis of compound 71.2. Compound 71.2 was prepared from compound 71.1 and 7-(4,4,5,5-tetramethyl-1,3,2-dioxaborolan-2-yl)imidazo[1,5-a]pyridine following the procedure described in the synthesis of compound 59.12. The product was purified by flash column chromatography on silica gel (58% ethyl acetate in hexane). MS(ES): m/z 615.4 [M+H]⁺.

Synthesis of compound 71.3. Compound 71.3 was prepared from compound 71.2 following the procedure described in the synthesis of compound 1.10. The product was triturated with n-pentane and dried under reduced pressure. It was used in the next step without purification. MS(ES): m/z 587.7 [M+H]⁺.

Synthesis of compound 71.4. Compound 71.4 was prepared from compound 71.3 following the procedure described in the synthesis of compound 1.11. The product was purified by flash column chromatography on silica gel (Combiflash®, 2.1% methanol in DCM). MS(ES): m/z 584.8 [M+H]⁺.

Synthesis of compound 71.5. Compound 71.5 was prepared from compound 71.4 following the procedure described in the synthesis of compound 1.12. The product was purified by flash column chromatography on silica gel (1.8% methanol in DCM). MS(ES): m/z 598.8 [M+H]⁺.

Synthesis of compound 71.6. Compound 71.6 was prepared from compound 71.5 following the procedure described in the synthesis of compound 1.14. The product was used in the next step without purification. MS(ES): m/z 498.7 [M+H]⁺.

Synthesis of compound I-71. Compound I-71 was prepared from compound 71.6 following the procedure described in the synthesis of compound I-1. The product was purified by flash column chromatography on silica gel (Combiflash®, 3.2% methanol in DCM). MS (ES): m/z 556.2 [M+H]⁺, ¹H-NMR (400 MHz, DMSO-d₆): δ 8.50-8.41 (m, 3H), 8.03 (s, 1H), 7.55 (s, 1H), 7.36 (s, 1H), 6.90-6.84 (m, 2H) 6.68-6.62 (m, 1H), 4.09-3.91 (m, 2H), 3.86 (s, 3H), 3.53 (m, 3H), 3.48 (s, 3H), 3.35 (s, 3H), 2.10-2.07 (m, 1H), 1.99 (s, 1H), 1.87-1.84 (bs, 1H), 1.53 (bs, 1H), 1.33-1.28 (m, 2H).

### Example I-72: methyl ((1R,3R)-3-(9-(3,4-dihydro-2H-benzo[b][1,4]oxazin-6-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

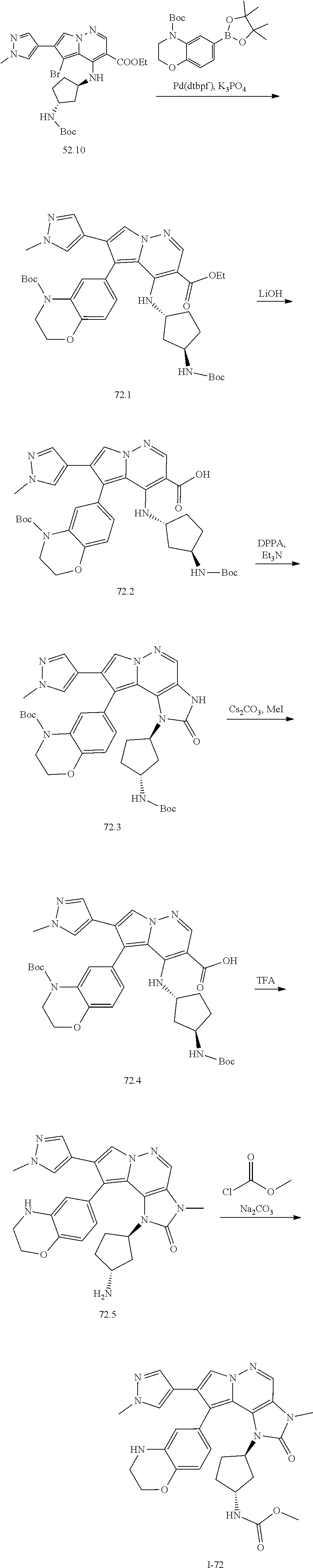

Synthesis of compound 72.1. Compound 72.1 was prepared from compound 59.10 and tert-butyl 6-(4,4,5,5-tetramethyl-1,3,2-dioxaborolan-2-yl)-2,3-dihydro-4H-benzo[b][1,4]oxazine-4-carboxylate following the procedure described in the synthesis of compound 59.12. The product was purified by flash column chromatography on silica gel (55% ethyl acetate in hexane). MS(ES): m/z 702.5 [M+H]⁺.

Synthesis of compound 72.2. Compound 72.2 was prepared from compound 72.1 following the procedure described in the synthesis of compound 1.10. The product was triturated with n-pentane and dried under reduced pressure. It was used in the next step without purification. MS(ES): m/z 674.7 [M+H]⁺.

Synthesis of compound 72.3. Compound 72.3 was prepared from compound 72.2 following the procedure described in the synthesis of compound 1.11. The product was purified by flash column chromatography on silica gel (Combiflash®, 2.1% methanol in DCM). MS(ES): m/z 671.8 [M+H]⁺.

Synthesis of compound 72.4. Compound 72.4 was prepared from compound 72.3 following the procedure described in the synthesis of compound 1.12. The product was purified by flash column chromatography on silica gel (1.8% methanol in DCM). MS(ES): m/z 685.8 [M+H]⁺.

Synthesis of compound 72.5. Compound 72.5 was prepared from compound 72.4 following the procedure described in the synthesis of compound 1.14. The product was used in the next step without purification. MS(ES): m/z 485.7 [M+H]⁺.

Synthesis of compound I-72. Compound I-72 was prepared from compound 72.5 following the procedure described in the synthesis of compound I-1. The product was purified by flash column chromatography on silica gel (Combiflash®, 3.2% methanol in DCM). MS (ES): m/z 543.3 [M+H]⁺, ¹H-NMR (400 MHz, DMSO-d₆): δ 8.33 (s, 1H), 8.07 (s, 1H), 7.36-7.35 (m, 1H), 7.18 (d, J=10.8 Hz, 1H), 6.94-6.92 (m, 0.5H) 6.85-6.83 (m, 0.5H), 6.80-6.74 (m, 1H), 6.49-6.44 (m, 2H), 5.90-5.87 (m, 1H), 4.24-4.21 (m, 2H), 4.17-4.15 (m, 2H), 4.11-4.06 (m, 1H), 3.50 (s, 3H), 3.45 (s, 3H), 3.34-3.32 (s, 3H), 2.08-2.05 (m, 1H), 1.92-1.87 (m, 2H), 1.60 (m, 1H), 1.46-1.43 (m, 1H), 1.33-1.28 (m, 1H), 0.92-0.85 (bs, 1H).

### Example I-73: methyl ((1R,3R)-3-(8-(3,6-dihydro-2H-pyran-4-yl)-3-methyl-9-(5-methyl-6-oxo-1,6-dihydropyridazin-3-yl)-2-oxo-2,3-dihydro-1H-imidazo[4,5-d]pyrrolo[1,2-b]pyridazin-1-yl)cyclopentyl)carbamate

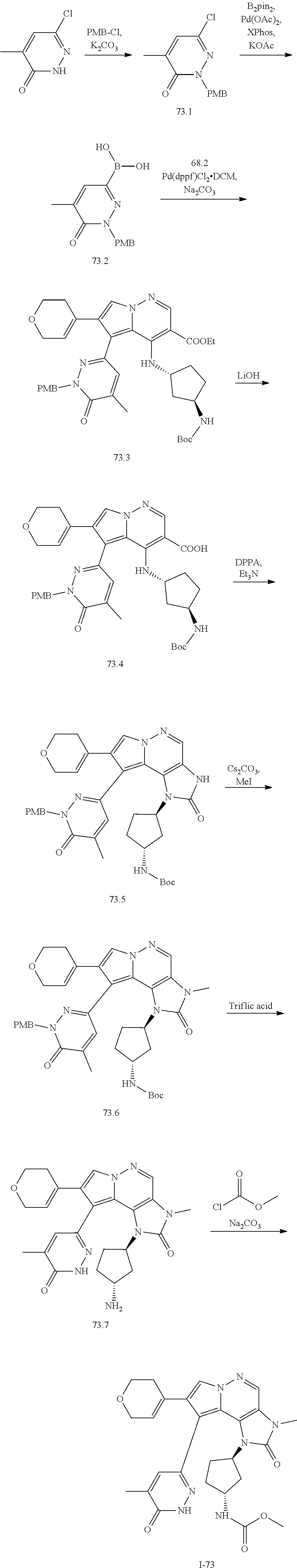

Synthesis of compound 73.1. To a solution of 6-chloro-4-methylpyridazin-3(2H)-one (1.0 g, 6.94 mmol, 1.0 equiv) in DMF (20 mL) was added potassium carbonate (1.43 g, 10.41 mmol, 1.5 equiv) followed by 4-methoxybenzyl chloride (1.3 g, 8.33 mmol, 1.2 equiv) at room temperature. The reaction mixture was stirred at room temperature for 16 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 35% ethyl acetate in hexane) to afford 73.1 (1.2 g, 65.5%). MS(ES): m/z 265.3 [M+H]⁺.

Synthesis of compound 73.2. A mixture of 73.1 (0.5 g, 1.89 mmol, 1.0 equiv), bis(pinacolato)diboron (2.88 g, 11.36 mmol, 6.0 equiv), potassium acetate (0.37 g, 3.78 mmol, 2.0 equiv), 2-dicyclohexylphosphino-2′,4′,6′-triisopropyl-1,1′-biphenyl (0.176 g, 0.37 mmol, 0.2 equiv) and palladium acetate (0.04 g 0.18 mmol, 0.1 equiv) in toluene (30 mL) was degassed with argon for 10 min. The reaction mixture was stirred at 80° C. for 1.5 h. It was transferred into ice-water and extracted with ethyl acetate. The combined organic layers were washed with brine, dried over anhydrous sodium sulfate, filtered and concentrated under reduced pressure to afford material 73.2 (0.9 g). MS(ES): m/z 275.6 [M+H]⁺.

Synthesis of compound 73.3. Compound 73.3 was prepared from compound 73.2 and 68.2 following the procedure described in the synthesis of compound 1.13. The product was purified by flash column chromatography on silica gel (52% ethyl acetate in hexane). MS(ES): m/z 699.6 [M+H]⁺.

Synthesis of compound 73.4. Compound 73.4 was prepared from compound 73.3 following the procedure described in the synthesis of compound 1.10. The product was triturated with pentane, dried under vacuum and used in the next step without purification. MS(ES): m/z 671.68 [M+H]⁺.

Synthesis of compound 73.5. Compound 73.5 was prepared from compound 73.4 following the procedure described in the synthesis of compound 1.11. The product was purified by flash column chromatography on silica gel (Combiflash®, 4.5% methanol in DCM). MS(ES): m/z 668.5 [M+H]⁺.

Synthesis of compound 73.6. Compound 73.6 was prepared from compound 73.5 following the procedure described in the synthesis of compound 1.12. The product was purified by flash column chromatography on silica gel (3.0% methanol in DCM). MS(ES): m/z 682.75

Synthesis of compound 73.7. To a solution of 73.6 (0.05 g, 0.073 mmol, 1.0 equiv) in DCM (2.5 mL) was added trifluoromethanesulfonic acid (1.0 mL) at 0° C. The reaction mixture was stirred at room temperature for 5 h. It was transferred into a saturated sodium bicarbonate solution and extracted with 20% methanol in DCM. The combined organic layers were washed with brine, dried over anhydrous sodium sulfate, filtered and concentrated under reduced pressure to afford 73.7 (0.045 g, 91%) MS(ES): m/z 462.37 [M+H]⁺.

Synthesis of I-73. Compound I-73 was prepared from compound 73.7 following the procedure described in the synthesis of compound I-1. The product was purified by flash column chromatography on silica gel (Combiflash®, 3.2% methanol in DCM). MS(ES): m/z 520.22 [M+H]⁺, ¹H-NMR (400 MHz, DMSO-d₆): δ 13.12 (s, 1H), 8.48 (s, 1H), 7.99 (s, 1H), 7.40 (s, 1H), 7.14-6.97 (m, 1H), 5.55 (s, 1H), 4.15-4.01 (m, 4H), 3.73 (s, 2H), 3.50 (s, 3H), 3.38 (s, 3H), 2.33 (s, 1H), 2.13 (s, 3H), 1.96 (s, 2H), 1.69 (s, 1H), 1.39 (s, 2H), 1.10-0.97 (m, 2H).

### Example I-74: (R)-2,2-difluoro-N-((1s,4S)-4-(8-(3-methoxy-1-methyl-1H-pyrazol-4-yl)-3-methyl-2-oxo-9-(pyrazolo[1,5-a]pyridin-5-yl)-2,3-dihydro-1H-imidazo[4,5-d]pyrrolo[1,2-b]pyridazin-1-yl)-1-methylcyclohexyl)cyclopropane-1-carboxamide

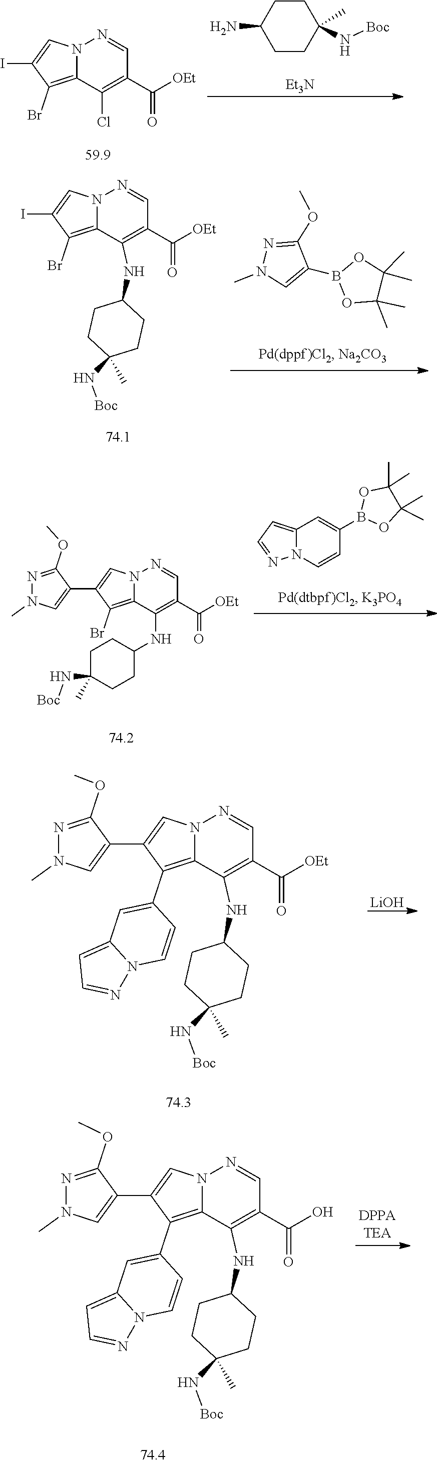

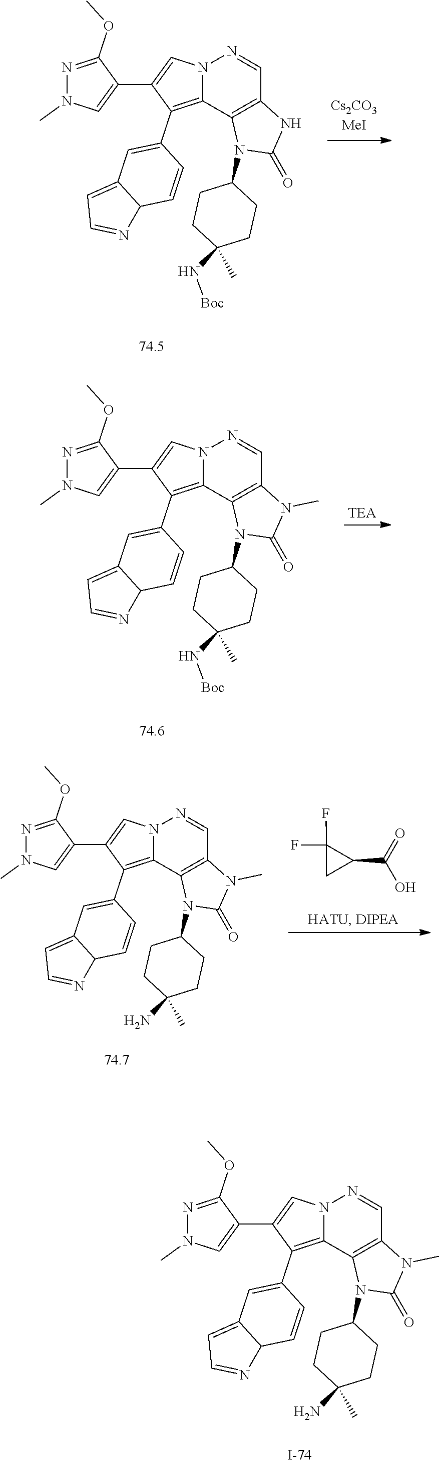

Synthesis of compound 74.1. Compound 74.1 was prepared from compound 59.9 and tert-butyl ((1s,4s)-4-amino-1-methylcyclohexyl)carbamate following the procedure described in the synthesis of compound 1.9. The product was purified by flash column chromatography on silica gel (Combiflash®, 0-15% ethyl acetate in hexane). MS(ES): m/z 621.5 and 623.5 [M+H]⁺.

Synthesis of compound 74.2. Compound 74.2 was prepared from compound 74.1 and 3-methoxy-1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (Combiflash®, 18% ethyl acetate in hexane). MS(ES): m/z 605.2 and 607.2 [M+H]⁺.

Synthesis of compound 74.3. Compound 74.3 was prepared from compound 74.2 and 5-(4,4,5,5-tetramethyl-1,3,2-dioxaborolan-2-yl)pyrazolo[1,5-a]pyridine following the procedure described in the synthesis of compound 59.12. The product was purified by flash column chromatography on silica gel (Combiflash®, 18% ethyl acetate in hexane). MS(ES): m/z 644.3 [M+H]⁺.

Synthesis of compound 74.4. Compound 74.4 was prepared from compound 74.3 following the procedure described in the synthesis of compound 1.10. The product was triturated with pentane, dried under reduced pressure and used without purification. MS(ES): m/z 615.2 [M+H]⁺.

Synthesis of compound 74.5. Compound 74.5 was prepared from compound 74.4 following the procedure described in the synthesis of compound 1.11. The product was purified by flash column chromatography on silica gel (Combiflash®, 0-3% methanol in DCM). MS(ES): m/z 612.5 [M+H]⁺.

Synthesis of compound 74.6. Compound 74.6 was prepared from compound 74.5 following the procedure described in the synthesis of compound 1.12. The product was purified by flash column chromatography on silica gel (0-3% methanol in DCM). MS(ES): m/z 626.1 [M+H]⁺.

Synthesis of compound 74.7. Compound 74.7 was prepared from compound 74.6 following the procedure described in the synthesis of compound 1.14. The product was used in the next step without purification. MS(ES): m/z 526 [M+H]⁺.

Synthesis of compound I-74. Compound I-74 was prepared from compound 74.7 and (1R,2R)-2-fluorocyclopropane-1-carboxylic acid following the procedure described in the synthesis of compound I-45. The product was purified by flash column chromatography on silica gel (0-3% methanol in DCM). MS(ES): m/z 630 [M+H]⁺, ¹H NMR (DMSO-d₆, 400 MHz): δ 8.83 (d, J=8.8 Hz, 1H), 8.42 (s, 1H), 8.11 (d, J=2.0 Hz, 1H), 8.04 (s, 1H), 7.73 (s, 1H), 7.59 (s, 1H), 6.86 (d, J=7.6 Hz, 1H), 6.75 (s, 1H), 6.67 (d, J=1.6 Hz, 1H), 3.83 (s, 3H), 3.51 (s, 3H), 3.38 (s, 3H), 3.28 (s, 3H), 1.99 (m, 2H), 1.24-1.07 (m, 5H), 0.75 (s, 3H), 1.12-0.92 (m, 1H), −0.51 (m, 1H).

### Example I-75: (1R,2R)-2-fluoro-N-((1s,4S)-1-methyl-4-(3-methyl-8-(1-methyl-1H-pyrazol-4-yl)-2-oxo-9-(thieno[2,3-c]pyridin-2-yl)-2,3-dihydro-1H-imidazo[4,5-d]pyrrolo[1,2-b]pyridazin-1-yl)cyclohexyl)cyclopropane-1-carboxamide

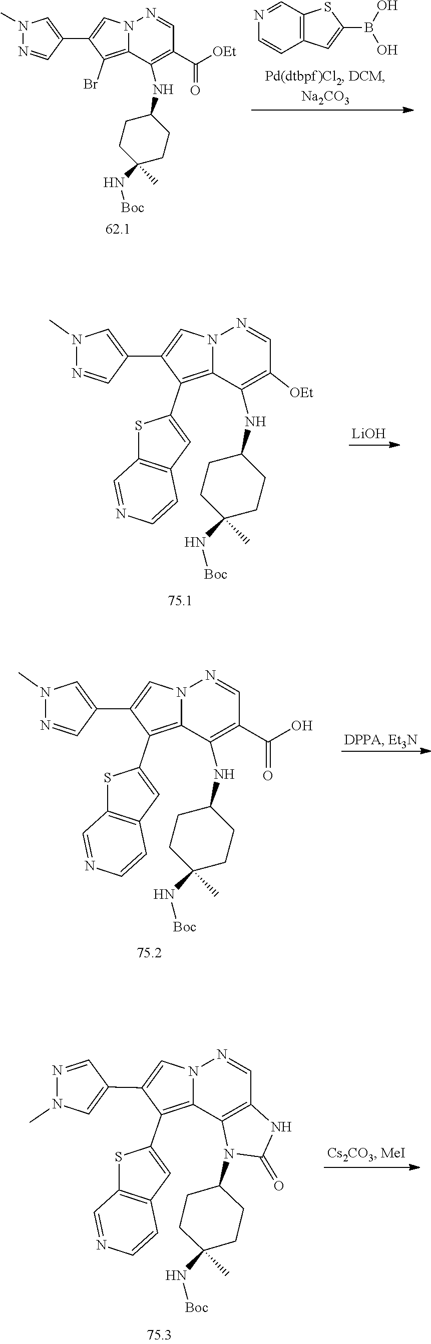

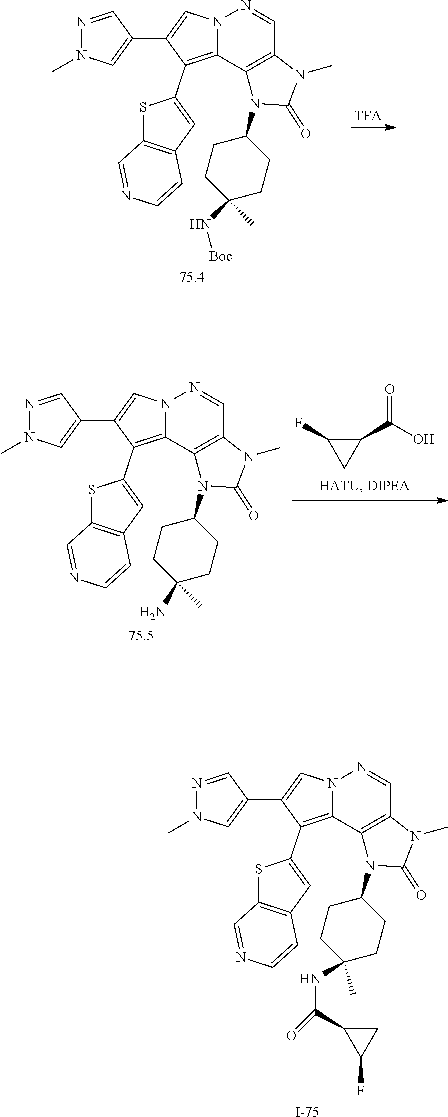

Synthesis of compound 75.1. Compound 75.1 was prepared from compound 62.1 and thieno[2,3-c]pyridin-2-ylboronic acid following the procedure described in the synthesis of compound 1.13. The product was purified by flash column chromatography on silica gel (24% ethyl acetate in n-hexane). MS (ES): m/z 630.8 [M+H]⁺.

Synthesis of compound 75.2. Compound 75.2 was prepared from compound 75.1 following the procedure described in the synthesis of compound 1.10. The product was used in the next step without purification. MS (ES): m/z 602.8 [M+H]⁺.

Synthesis of compound 75.3. Compound 75.3 was prepared from compound 75.2 following the procedure described in the synthesis of compound 1.11. The product was purified by flash column chromatography on silica gel (2.5% methanol in DCM). MS (ES): m/z 599.7 [M+H]⁺.

Synthesis of compound 75.4. Compound 75.4 was prepared from compound 75.3 following the procedure described in the synthesis of compound 1.12. The product was purified by flash column chromatography on silica gel (2.8% methanol in DCM). MS (ES): m/z 613.9 [M+H]⁺.

Synthesis of compound 75.5. Compound 75.5 was prepared from compound 75.4 following the procedure described in the synthesis of compound 1.14. The product was used in the next step without purification. MS (ES): m/z 513.5 [M+H]⁺.

Synthesis of compound I-75. Compound I-75 was prepared from compound 75.5 and (1R,2R)-2-fluorocyclopropane-1-carboxylic acid following the procedure described in the synthesis of compound I-45. The product was purified by flash column chromatography on silica gel (3.5% methanol in DCM). MS (ES): m/z 599.2 [M+H]⁺, ¹H NMR (DMSO-d₆, 400 MHz): δ 9.32 (s, 1H), 8.57 (d, J=5.2 Hz, 1H), 8.51 (d, J=Hz, 1H), 8.21 (s, 1H), 7.92 (dd, J=4.8 Hz, 1H), 7.68 (s, 1H), 7.45 (s, 1H), 7.36 (s, 1H), 7.17 (s, 1H), 4.77-4.06 (m, 1H), 3.70 (s, 3H), 3.35 (s, 3H), 2.34 (d, J=12.8 Hz, 1H), 1.88 (m, 1H), 1.77-1.79 (m, 2H), 1.33 (m, 1H), 1.23 (m, 2H), 1.15-1.17 (m, 1H), 0.90 (t, J=16.8 Hz, 2H), 0.54 (s, 3H).

### Example I-76: (1s,3S)-3-(but-2-ynamido)cyclobutyl ((1R,3R)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

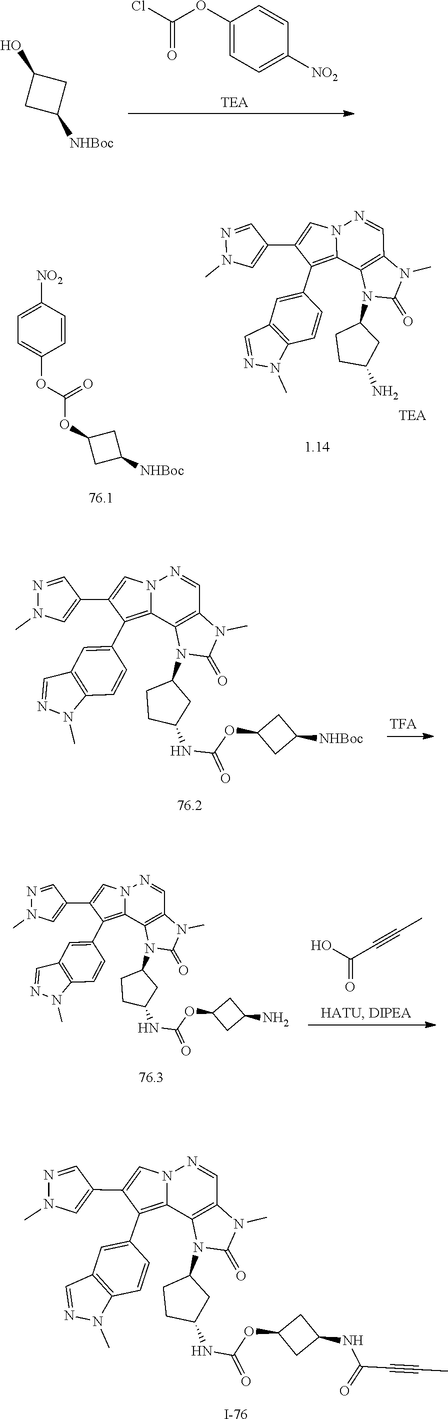

Synthesis of compound 76.1. To a solution of tert-butyl ((1s,3s)-3-hydroxycyclobutyl)carbamate (1.0 g, 5.34 mmol, 1.0 equiv) and 4-nitrophenyl carbonochloridate (1.28 g, 6.41 mmol, 1.0 equiv) in DCM (20 mL) was added triethylamine (1.48 mL, 10.68 mmol, 2.0 equiv) and the reaction mixture was stirred at room temperature for 1 h. It was transferred into water, extracted with DCM, organic layer was washed with saturated sodium bicarbonate solution followed by brine, dried over anhydrous sodium sulfate, filtered and concentrated under reduced pressure to afford 76.1 (1.1 g).

Synthesis of compound 76.2. To a solution of 1.14 (0.250 g, 0.51 mmol, 1.0 equiv) in THF (8 mL) was cooled to 0° C. and added triethylamine (0.21 mL, 1.55 mmol, 3.0 equiv) followed by solution of 76.1 (0.274 g, 0.77 mmol, 1.5 equiv) in THF. The reaction mixture was stirred at room temperature for 1 h. It was transferred into water and extracted with ethyl acetate. The combined organic layers were washed with brine, dried over anhydrous sodium sulfate, filtered and concentrated under reduced pressure. The residue was purified by silica gel flash column chromatography on silica gel (2.8% methanol in DCM) to afford 76.2 (0.160 g, 32.5%). MS(ES): m/z 695.54 [M+H]⁺.

Synthesis of compound 76.3. To a solution of 76.2 (0.160 g, 0.19 mmol, 1.0 equiv) in DCM (5.0 mL) at 0° C. was added trifluoroacetic acid (1.5 mL) dropwise and stirred for 15 min at room temperature. It was quenched with saturated sodium bicarbonate solution and extracted with DCM. The combined organic layers were washed with brine, dried over anhydrous sodium sulfate, filtered and concentrated under reduced pressure to afford 76.3 (0.125 g). MS(ES): m/z 595.3 [M+H]⁺.

Synthesis of I-76. Compound I-76 was prepared from compound 76.3 and but-2-ynoic acid following the procedure described in the synthesis of compound I-45. The product was purified by silica gel flash column chromatography on silica gel (4.3% methanol in DCM). MS(ES): m/z 661.14 [M+H]⁺, ¹H-NMR (400 MHz, DMSO-d₆): δ 8.79 (d, J=7.6 Hz, 1H), 8.39 (s, 1H), 8.16-8.06 (m, 2H), 7.78-7.75 (m, 2H), 7.42-7.38 (m, 1H), 7.27 (d, J=7.2 Hz, 1H), 7.05 (s, 1H), 6.79-6.77 (m, 1H), 4.53-4.44 (m, 1H), 4.14 (s, 3H), 3.87-3.81 (m, 2H), 3.66 (s, 3H), 3.38 (s, 3H), 2.59 (s, 2H), 2.02-1.69 (m, 7H), 1.49-1.03 (m, 3H), 0.78 (s, 1H), −0.18-−0.20 (m, 1H).

### Example I-77: methyl ((1R,3R)-3-(8-(3,6-dihydro-2H-pyran-4-yl)-9-(1H-indazol-6-yl)-3-methyl-2-oxo-2,3-dihydro-1H-imidazo[4,5-d]pyrrolo[1,2-b]pyridazin-1-yl)cyclopentyl)carbamate

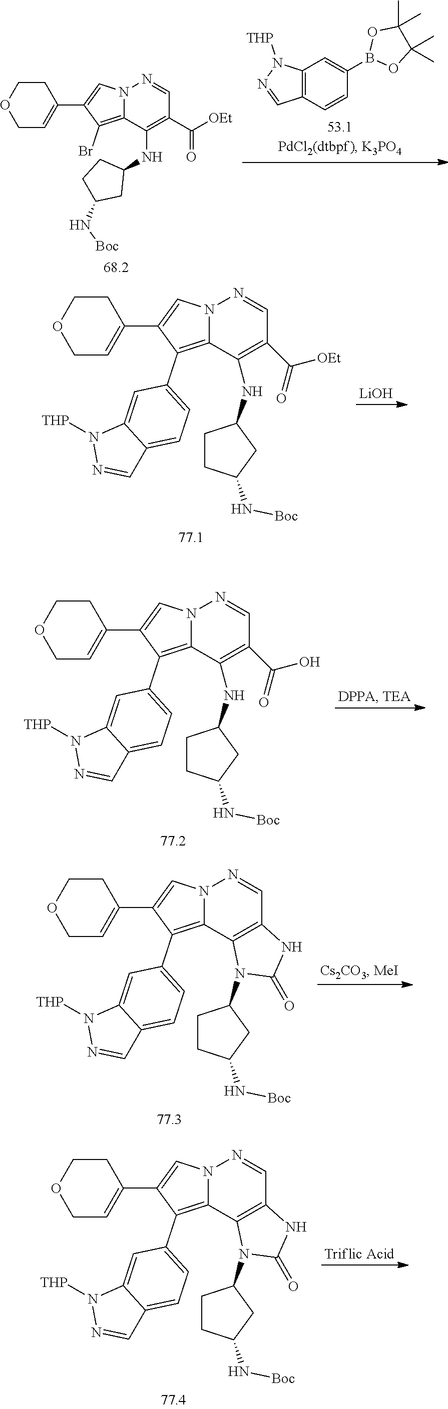

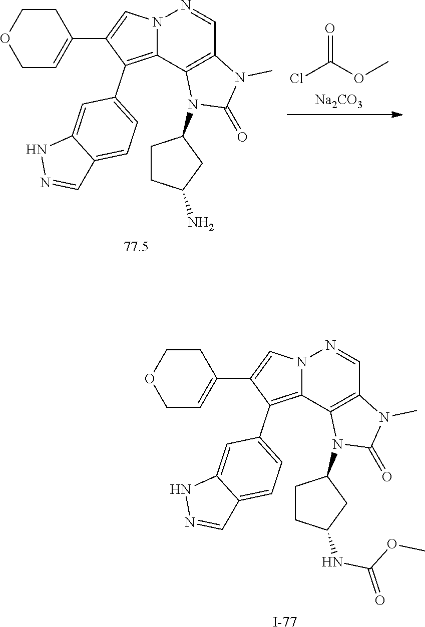

Synthesis of compound 77.1. Compound 77.1 was prepared from compound 68.2 and 53.1 following the procedure described in the synthesis of compound 59.12. The product was purified by flash column chromatography on silica gel (50% ethyl acetate in hexane). MS(ES): m/z 621.28 [M+H]⁺.

Synthesis of compound 77.2. Compound 77.2 was prepared from compound 77.1 following the procedure described in the synthesis of compound 1.10. The product was triturated with pentane, dried under vacuum and used in the next step without purification. MS(ES): m/z 593.31 [M+H]⁺.

Synthesis of compound 77.3. Compound 77.3 was prepared from compound 77.2 following the procedure described in the synthesis of compound 1.11. The product was purified by flash column chromatography on silica gel (Combiflash®, 1.8% methanol in DCM). MS(ES): m/z 590.3 [M+H]⁺.

Synthesis of compound 77.4. Compound 77.4 was prepared from compound 77.3 following the procedure described in the synthesis of compound 1.12. The product was purified by flash column chromatography on silica gel (1.4% methanol in DCM. MS(ES): m/z 604.33 [M+H]⁺.

Synthesis of compound 77.5. Compound 77.5 was prepared from compound 77.3 following the procedure described in the synthesis of compound 21.8. The product was used in the next step without purification. MS(ES): m/z 470.33 [M+H]⁺.

Synthesis of I-77. Compound I-77 was prepared from compound 77.4 following the procedure described in the synthesis of compound I-1. The product was purified by flash column chromatography on silica gel (Combiflash®, 3.7% methanol in DCM). MS(ES): m/z 528.3 [M+H]⁺, ¹H-NMR (400 MHz, DMSO-d₆): δ 13.08 (s, 1H), 8.41 (s, 1H), 8.13 (s, 1H), 7.96 (s, 1H), 7.88-7.86 (m, 1H), 7.50-7.48 (m, 1H), 7.14-7.09 (m, 1H), 6.64-6.21 (m, 1H), 5.40 (d, J=17.2 Hz, 1H), 3.89 (s, 3H), 3.63 (d, J=4.8 Hz, 2H), 3.50-3.44 (m, 3H), 3.34 (s, 3H), 2.20 (s, 3H), 1.93 (s, 1H), 1.75 (m, 2H), 1.47 (m, 1H), 1.07-1.00 (m, 1H), 0.85 (m, 1H).

### Example I-78: 1-((1s,4s)-4-hydroxy-4-methylcyclohexyl)-9-(1H-indazol-6-yl)-3-methyl-8-(2-methyl-2H-1,2,3-triazol-4-yl)-1,3-dihydro-2H-imidazo[4,5-d]pyrrolo[1,2-b]pyridazin-2-one

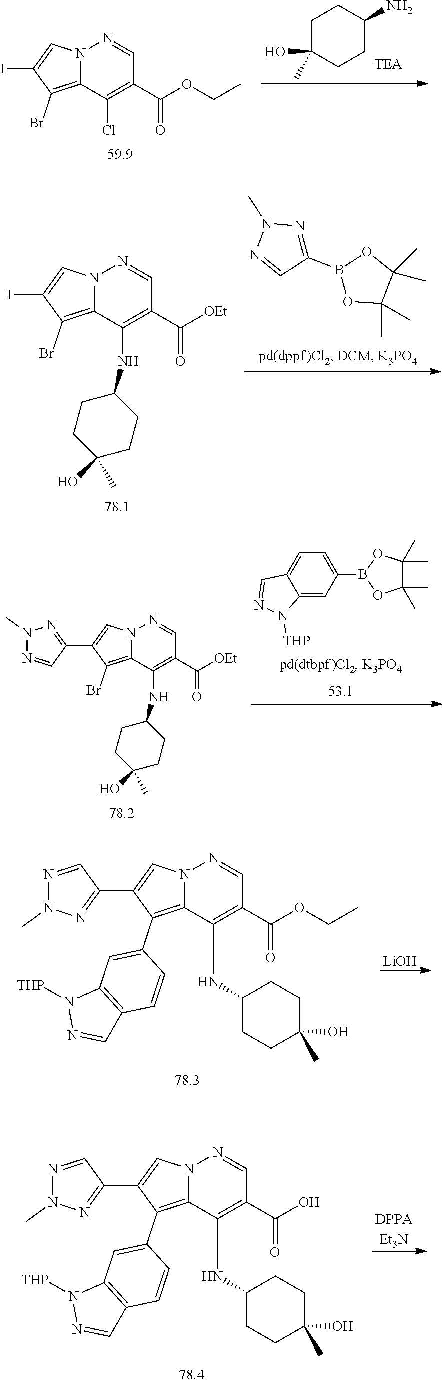

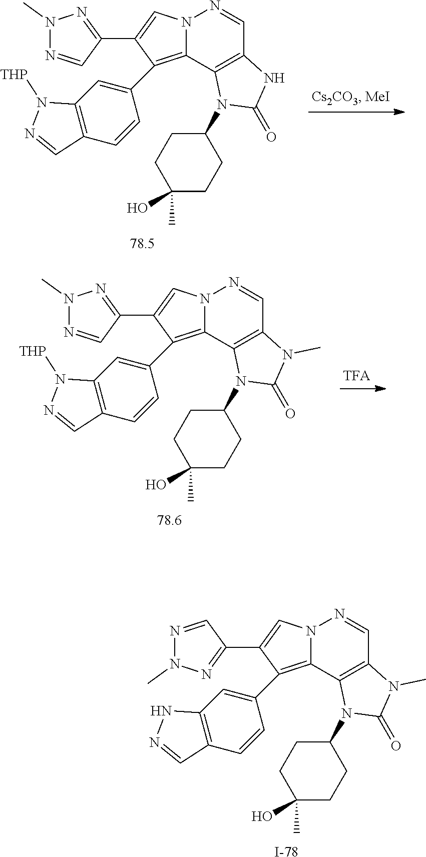

Synthesis of compound 78.1. Compound 78.1 was prepared from compound 59.9 and 2-methyl-4-(4,4,5,5-tetramethyl-1,3,2-dioxaborolan-2-yl)-2H-1,2,3-triazole following the procedure described in the synthesis of compound 1.9. The product was purified by flash column chromatography on silica gel (Combiflash®, 15% ethyl acetate in hexane). MS(ES): m/z 522.5 and 524.4 [M+H]⁺.

Synthesis of compound 78.2. Compound 78.2 was prepared from compound 78.1 and (1s,4s)-4-amino-1-methylcyclohexan-1-ol following the procedure described in the synthesis of compound 54.3. The product was purified by flash column chromatography on silica gel (Combiflash®, 30% ethyl acetate in hexane). MS(ES): m/z 577.2 and 579.4 [M+H]⁺.

Synthesis of compound 78.3. Compound 78.3 was prepared from compound 78.2 and 53.1 following the procedure described in the synthesis of compound 59.12. The product was purified by flash column chromatography on silica gel (Combiflash®, 18% ethyl acetate in hexane). MS(ES): m/z 599.6 [M+H]⁺.

Synthesis of compound 78.4. Compound 78.4 was prepared from compound 78.3 following the procedure described in the synthesis of compound 1.10. The product was triturated with pentane, dried under vacuum and used in the next step without purification. MS(ES): m/z 571.4 [M+H]⁺.

Synthesis of compound 78.5. Compound 78.5 was prepared from compound 78.4 following the procedure described in the synthesis of compound 1.11. The product was purified by flash column chromatography on silica gel (Combiflash®, 0-3% methanol in DCM). MS(ES): m/z 568.5 [M+H]⁺.

Synthesis of compound 78.6. Compound 78.6 was prepared from compound 78.5 following the procedure described in the synthesis of compound 1.12. The product was purified by flash column chromatography on silica gel (3% methanol in DCM). MS(ES): m/z 582.7 [M+H]⁺.

Synthesis of compound I-78. To a solution of 78.6 (0.032 g, 0.055 mmol, 1.0 equiv) in DCM (4 mL) at 0° C. was added trifluoroacetic acid (0.5 mL) dropwise and stirred for 30 min at room temperature. The reaction mixture was concentrated under reduced pressure. The reaction mixture was basified to pH-9 by sodium bicarbonate solution and extracted with ethyl acetate. The combined organic layers were washed with brine, dried over anhydrous sodium sulfate, filtered and concentrated under reduced pressure. The residue was purified by flash column chromatography on silica gel (0-3% methanol in DCM) to afford I-78 (0.013 g, 47.5%). MS(ES): m/z 498.3 [M+H]⁺, ¹H-NMR (400 MHz, DMSO-d₆): δ 13.12 (s, 1H), 8.48 (s, 1H), 8.19 (m, 2H), 7.91 (d, J=8.4 Hz, 1H), 7.53 (s, 1H), 7.15 (d, J=9.2 Hz, 1H), 6.47 (s, 1H), 4.06 (s, 3H), 3.86 (m, 1H), 3.33 (s, 3H), 2.22-2.19 (m, 2H), 0.98 (m, 4H), 0.85 (m, 3H), 0.40 (m, 1H), −0.07 (m, 1H), -0.45 (m, 1H).

### Example I-79: methyl ((1R,3R)-3-(9-(1H-indazol-3-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

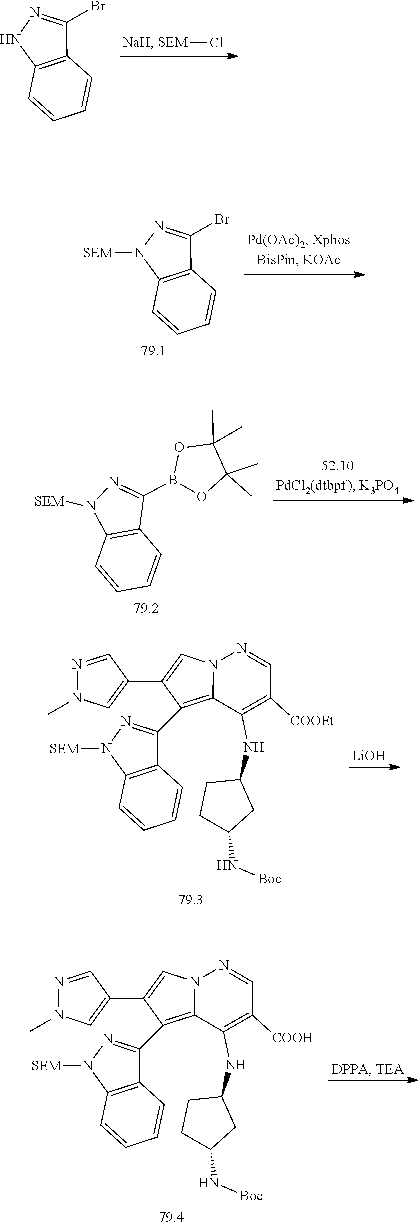

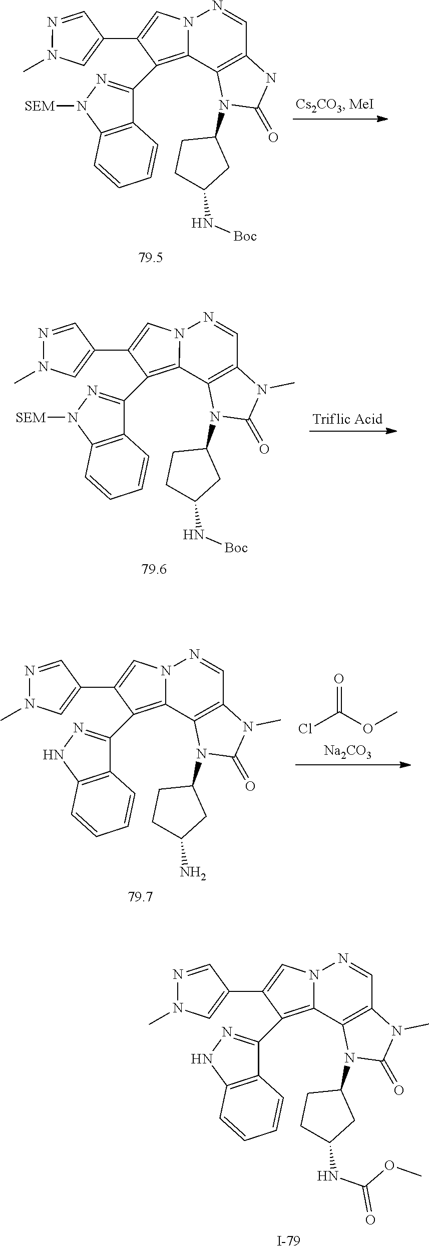

Synthesis of compound 79.1. To a solution of 3-bromo-1H-indazole (2.0 g, 10.15 mmol, 1.0 equiv) in DMF (40 mL) was added sodium hydride (60% dispersion in mineral oil, 0.812 g, 20.30 mmol, 2.0 equiv) at 0° C. and stirred for 30 min. To the mixture was added 2-(trimethylsilyl) ethoxy methyl chloride (2.53 g, 15.22 mmol, 1.5 equiv). The reaction mixture was stirred at room temperature for 2 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 8% ethyl acetate in hexane) to afford 79.1 (1.8 g, 54%). MS(ES): m/z 327.2 and 329.2 [M+H]⁺.

Synthesis of compound 79.2. A mixture of 79.1 (1.8 g, 5.50 mmol, 1.0 equiv), bis(pinacolato)diboron (2.78 g, 11.01 mmol, 2.0 equiv) and potassium acetate (1.62 g, 16.51 mmol, 3.0 equiv) in toluene (18 mL) was degassed by bubbling through a stream of argon for 10 min. 2-Dicyclohexylphosphino-2′,4′,6′-triisopropyl-1,1′-biphenyl (0.52 g, 1.10 mmol, 0.1 equiv) and palladium (II) acetate (0.370 g, 0.55 mmol, 0.1 equiv) were added and degassed for 5 min. The reaction mixture was stirred at 90° C. for 4 h. It was transferred into water and extracted with ethyl acetate. The combined organic layers were washed with brine, dried over anhydrous sodium sulfate, filtered and concentrated under reduced pressure. The residue was purified by flash column chromatography on silica gel (10% ethyl acetate in hexane) to afford 79.2 (0.82 g, 40%). MS(ES): m/z 375.2 [M+H]⁺.

Synthesis of compound 79.3. Compound 79.3 was prepared from compound 79.2 and 52.10 following the procedure described in the synthesis of compound 59.12. The product was purified by flash column chromatography on silica gel (42% ethyl acetate in hexane). MS(ES): m/z 715.4 [M+H]⁺.

Synthesis of compound 79.4. Compound 79.4 was prepared from compound 79.3 following the procedure described in the synthesis of compound 1.10. The product was triturated with pentane, dried under vacuum and used in the next step without purification. MS(ES): m/z 687.2 [M+H]⁺.

Synthesis of compound 79.5. Compound 79.5 was prepared from compound 79.4 following the procedure described in the synthesis of compound 1.11. The product was purified by flash column chromatography on silica gel (Combiflash®, 1.6% methanol in DCM). MS(ES): m/z 684.2 [M+H]⁺.

Synthesis of compound 79.6. Compound 79.6 was prepared from compound 79.5 following the procedure described in the synthesis of compound 1.12. The product was purified by flash column chromatography on silica gel (1.2% methanol in DCM). MS(ES): m/z 698.1 [M+H]⁺.

Synthesis of compound 79.7. Compound 79.7 was prepared from compound 79.6 following the procedure described in the synthesis of compound 21.8. The product was used in the next step without purification. MS(ES): m/z 468.2 [M+H]⁺.

Synthesis of I-79. Compound I-79 was prepared from compound 79.7 following the procedure described in the synthesis of compound I-1. The product was purified by flash column chromatography on silica gel (Combiflash®, 2.6% methanol in DCM). MS(ES): m/z 526.2 [M+H]⁺, ¹H-NMR (400 MHz, DMSO-d₆): δ 13.25 (s, 1H), 8.46 (s, 1H), 8.24 (s, 1H), 7.64 (d, J=7.6 Hz, 1H), 7.40-7.36 (m, 2H), 7.24 (d, J=8.0 Hz, 1H), 7.06-7.02 (m, 2H), 6.75 (bs, 0.5H), 6.37 (bs, 0.5H), 3.88 (s, 1H), 3.66 (s, 3H), 3.49 (s, 3H), 3.35 (s, 3H), 2.02 (bs, 1H), 1.78 (bs, 1H), 1.55 (bs, 2H), 1.23-1.18 (m, 1H), 0.84-0.75 (m, 1H), 0.28-0.09 (m, 1H).

### Example I-80: methyl ((1R,3R)-3-(9-(7-fluoro-1H-indazol-6-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

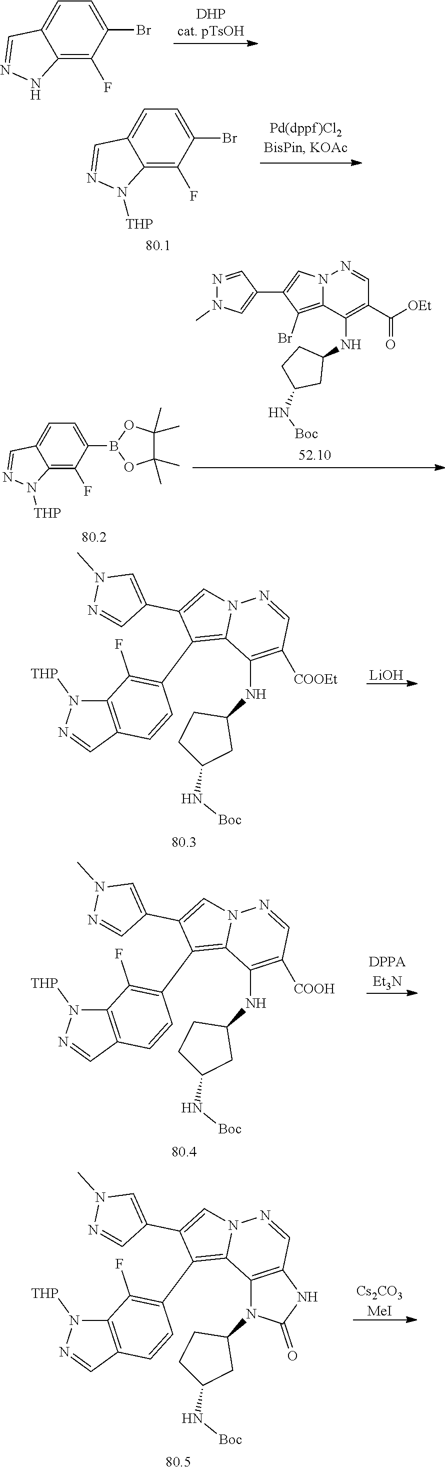

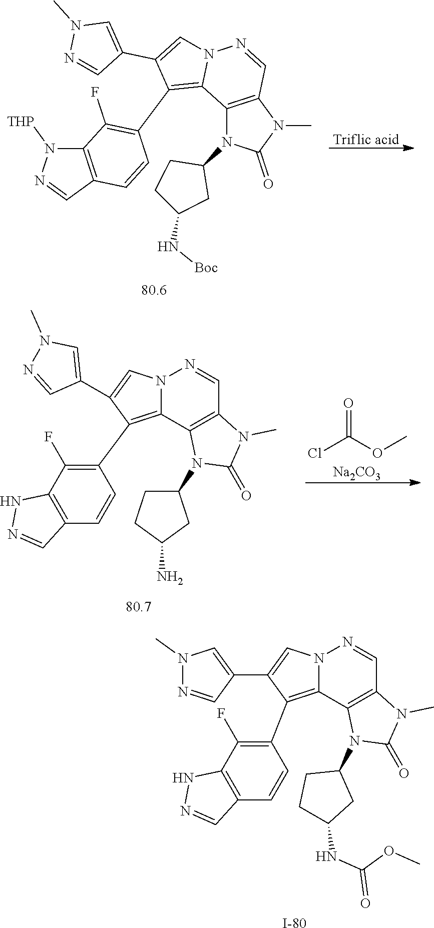

Synthesis of compound 80.1. To a solution of 6-bromo-4-fluoro-1H-indazole (4 g, 18.6 mmol, 1 equiv) in DCM (40 mL) was added 3,4-dihydro-2H-pyran (2.3 g, 27.9 mmol, 1.5 equiv) and p-toluene sulfonic acid monohydrate (353 mg, 1.86 mmol, 0.1 equiv) at 0° C. and stirred at room temperature for 16 h. It was transferred into water and extracted with ethyl acetate. The combined organic layers were washed with brine, dried over anhydrous sodium sulfate, filtered and concentrated under reduced pressure. The residue was purified by flash column chromatography on silica gel (20% ethyl acetate in hexane) to afford 80.1 (3.8 g, 71%). MS(ES): m/z 299 and 301 [M+H]⁺.

Synthesis of compound 80.2 A mixture of 80.1 (3.8 g, 12.7 mmol, 1.0 equiv), bis(pinacolato)diboron (6.45 g, 25.41 mmol, 2.0 equiv) and potassium acetate (3.7 g, 38.1 mmol, 3.0 equiv) in dioxane (40 mL) was degassed by bubbling through a stream of argon for 10 min. 1,1′-Bis(diphenylphosphino)ferrocene]dichloropalladium (II) (0.928 g, 1.27 mmol, 0.1 equiv) was added and degassed for 5 min. The reaction mixture was stirred at 110° C. for 4 h. It was transferred into water and extracted with ethyl acetate. The combined organic layers were washed with brine, dried over anhydrous sodium sulfate, filtered and concentrated under reduced pressure. The residue was purified by flash column chromatography on silica gel (30% ethyl acetate in hexane) to afford 80.2 (2.4 g, 55%). MS(ES): m/z 347 [M+H]⁺.

Synthesis of compound 80.3. Compound 80.3 was prepared from compound 80.2 and 52.10 following the procedure described in the synthesis of compound 59.12. The product was purified by flash column chromatography on silica gel (Combiflash®, 65% ethyl acetate in hexane). MS(ES): m/z 687.7 [M+H]⁺.

Synthesis of compound 80.4. Compound 80.4 was prepared from compound 80.3 following the procedure described in the synthesis of compound 1.10. The product was triturated with pentane, dried under vacuum and used in the next step without purification. MS(ES): m/z 659.5 [M+H]⁺.

Synthesis of compound 80.5. Compound 80.5 was prepared from compound 80.4 following the procedure described in the synthesis of compound 1.11. The product was purified by flash column chromatography on silica gel (Combiflash®, 4.5% methanol in DCM). MS(ES): m/z 656.3 [M+H]⁺.

Synthesis of compound 80.6. Compound 80.6 was prepared from compound 80.5 following the procedure described in the synthesis of compound 1.12. The product was purified by flash column chromatography on silica gel (3.2% methanol in DCM). MS(ES): m/z 670.5 [M+H]⁺.

Synthesis of compound 80.7. Compound 80.7 was prepared from compound 80.6 following the procedure described in the synthesis of compound 21.8. MS(ES): m/z 485.3 [M+H]⁺. It was used in next step without purification.

Synthesis of I-80. Compound I-80 was prepared from compound 80.7 following the procedure described in the synthesis of compound I-1. The product was purified by flash column chromatography on silica gel (Combiflash®, 3.8% methanol in DCM). MS(ES): m/z 544.5 [M+H]⁺, ¹H NMR (DMSO-d₆, 400 MHz): δ 13.70-13.64 (m, 1H), 8.45 (s, 1H), 8.26 (s, 1H), 8.23-8.21 (m, 1H), 7.74-7.72 (m, 1H), 7.32-7.30 (m, 1H), 7.12-7.08 (m, 2H), 6.71-6.44 (m, 1H), 3.96-3.89 (m, 1H), 3.67 (s, 3H), 3.52 (s, 3H), 3.37 (s, 3H), 2.15-2.11 (m, 1H), 1.92-1.85 (m, 1H), 1.79 (bs, 1H), 1.55 (bs, 1H), 1.23 (s, 1H), 1.10-1.06 (m, 1H), 0.08 (bs, 1H).

### Example I-81: methyl ((1R,3R)-3-(9-(6,7-dihydro-4H-thieno[3,2-c]pyran-2-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

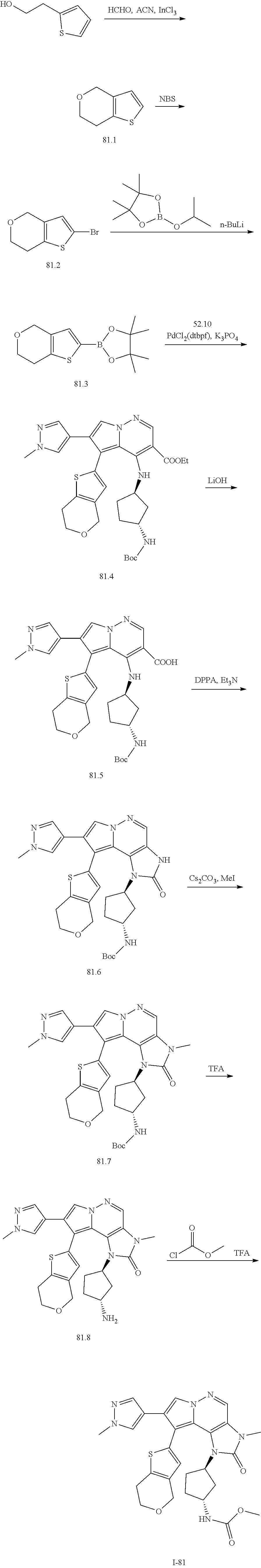

Synthesis of compound 81.1. A mixture of 2-(thiophen-2-yl)ethan-1-ol (5.0 g, 39.06 mmol, 1.0 equiv), paraformaldehyde (1.4 g, 46.87 mmol, 1.2 equiv) and indium chloride (0.431 g, 1.95 mmol, 0.05 equiv) in acetonitrile (400 mL) was stirred at 80° C. for 2 h. It was concentrated under reduced pressure. The residue was purified by vacuum distillation at 80-95° C. (8 mm Hg pressure) to afford 81.1 (2.1 g, 38%), MS(ES): m/z 141.3 [M+H]⁺.

Synthesis of compound 81.2. To a stirred solution of 81.1 (2.1 g, 15.0 mmol, 1.0 equiv) in toluene (20 mL) was added N-bromosuccinimide (2.67 g, 15 mmol, 1.0 equiv). The reaction mixture was stirred at room temperature for 16 h. It was transferred into 4% NaOH solution and stirred for 20 min and extracted with ethyl acetate. The combined organic layers were washed with brine, dried over anhydrous sodium sulfate, filtered and concentrated under reduced pressure. The residue was purified by flash column chromatography on silica gel (20% ethyl acetate in hexane) to afford 81.2 (1.4 g, 43%), MS(ES): m/z 220.2 [M+H]⁺.

Synthesis of compound 81.3. To a stirred solution of 81.2 (1.4 g, 6.39 mmol, 1.0 equiv) in THF (2 mL) at −78° C. was added n-BuLi (2.5 M in hexanes, 4.0 mL, 10.22 mmol, 1.6 equiv). The reaction mixture was stirred for 20 min at −78° C. Isopropoxy 4,4,5,5-tetramethyl-1,3,2-dioxaborolane (1.42 g, 7.67 mmol, 1.2 equiv) was added and reaction mixture was stirred for 30 min. It was transferred into water and extracted with ethyl acetate. The combined organic layers were washed with brine, dried over anhydrous sodium sulfate, filtered and concentrated under reduced pressure. The residue was purified by flash column chromatography on silica gel (20% ethyl acetate in hexane) to afford 81.3 (0.580 g, 34%). MS(ES): m/z 267.6 [M+H]⁺.

Synthesis of compound 81.4. Compound 81.4 was prepared from compound 81.3 following the procedure described in the synthesis of compound 59.12. The product was purified by flash column chromatography on silica gel (55% ethyl acetate in hexane). MS(ES): m/z 607.4 [M+H]⁺.

Synthesis of compound 81.5. Compound 81.5 was prepared from compound 81.4 following the procedure described in the synthesis of compound 1.10. The product was triturated with pentane, dried under vacuum and used in the next step without purification. MS(ES): m/z 579.3 [M+H]⁺.

Synthesis of compound 81.6. Compound 81.6 was prepared from compound 81.5 following the procedure described in the synthesis of compound 1.11. The product was purified by flash column chromatography on silica gel (Combiflash®, 2.1% methanol in DCM). MS(ES): m/z 576.3 [M+H]⁺.

Synthesis of compound 81.7. Compound 81.7 was prepared from compound 81.6 following the procedure described in the synthesis of compound 1.12. The product was purified by flash column chromatography on silica gel (1.8% methanol in DCM). MS(ES): m/z 590.3 [M+H]⁺.

Synthesis of compound 81.8. Compound 81.8 was prepared from compound 81.7 following the procedure described in the synthesis of compound 1.14. The product was used in the next step without purification. MS(ES): m/z 490.3 [M+H]⁺.

Synthesis of compound I-81. Compound I-81 was prepared from compound 81.8 following the procedure described in the synthesis of compound I-1. The product was purified by flash column chromatography on silica gel (Combiflash®, 3.2% methanol in DCM). MS (ES): m/z 548.3 [M+H]⁺, ¹H-NMR (400 MHz, DMSO-d₆): δ 8.43 (s, 1H), 8.13 (s, 1H), 7.52 (s, 1H), 7.27 (s, 1H), 7.09 (d, J=6.4 Hz, 1H), 4.70 (bs, 2H), 4.59-4.43 (m, 1H), 4.17-4.15 (m, 2H), 4.11-4.09 (m, 2H), 3.89 (bs, 1H), 3.77 (s, 3H), 3.51 (s, 3H), 3.36 (s, 3H), 2.96 (bs, 2H), 2.12 (bs, 1H), 1.98-1.95 (m, 1H), 1.62 (s, 1H), 1.39 (s, 1H), 0.96 (s, 1H).

### Example I-82: 2-fluoro-4-(1-((1s,4s)-4-hydroxy-4-methylcyclohexyl)-3-methyl-8-(1-methyl-1H-pyrazol-4-yl)-2-oxo-2,3-dihydro-1H-imidazo[4,5-d]pyrrolo[1,2-b]pyridazin-9-yl)-N-methylbenzamide

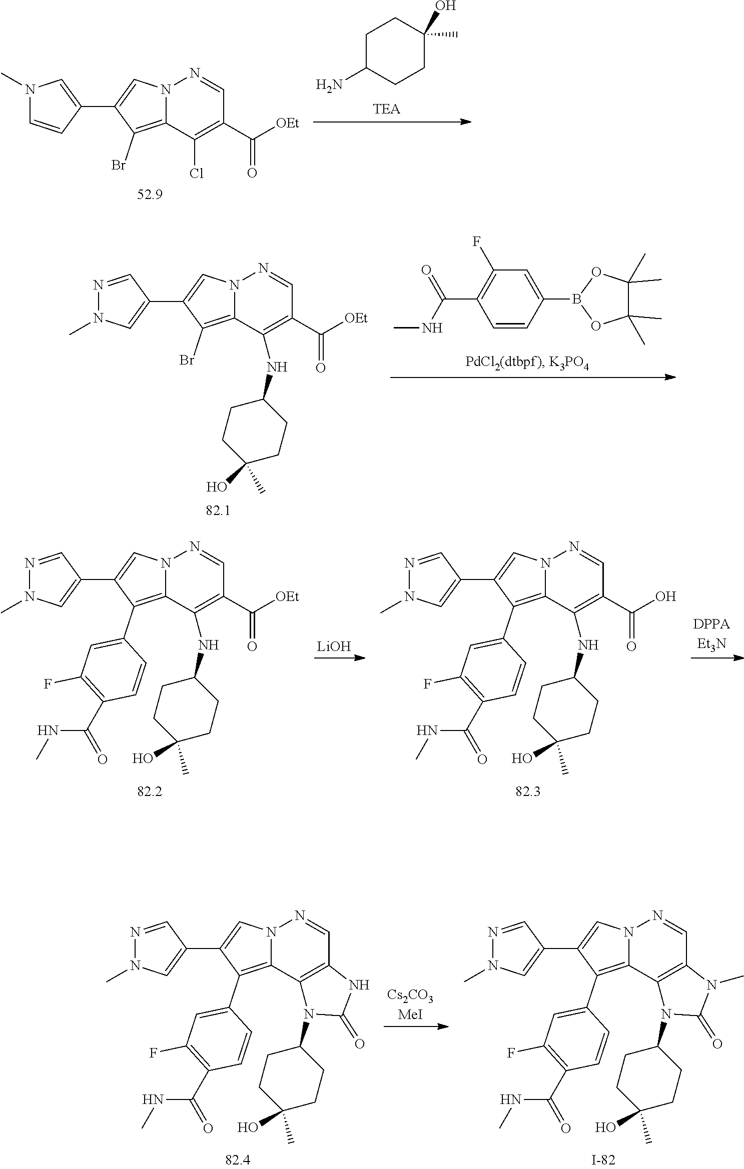

Synthesis of compound 82.1. Compound 82.1 was prepared from compound 52.9 and 4-amino-1-methylcyclohexan-1-ol following the procedure described in the synthesis of compound 1.9. The product was purified by flash column chromatography on silica gel (Combiflash®, 15% ethyl acetate in hexane). MS(ES): m/z 476.8 and 478.8 [M+H]⁺.

Synthesis of compound 82.2. Compound 82.2 was prepared from compound 82.1 and 2-fluoro-N-methyl-4-(4,4,5,5-tetramethyl-1,3,2-dioxaborolan-2-yl)benzamide following the procedure described in the synthesis of compound 59.12. The product was purified by flash column chromatography on silica gel (40% ethyl acetate in hexane). MS(ES): m/z 549.5 [M+H]⁺.

Synthesis of compound 82.3. Compound 82.3 was prepared from compound 82.2 following the procedure described in the synthesis of compound 1.10. The product was triturated with pentane, dried under vacuum and used in the next step without purification. MS(ES): m/z 521.2 [M+H]⁺.

Synthesis of compound 82.4. Compound 82.4 was prepared from compound 82.3 following the procedure described in the synthesis of compound 1.11. The product was purified by flash column chromatography on silica gel (Combiflash®, 1.6% methanol in DCM). MS(ES): m/z 518.57 [M+H]⁺.

Synthesis of compound I-82. Compound I-82 was prepared from compound 82.4 following the procedure described in the synthesis of compound 1.12. The product was purified by flash column chromatography on silica gel (1.2% methanol in DCM). MS(ES): m/z 532.3 [M+H]⁺, ¹H-NMR (400 MHz, DMSO-d₆): δ 8.42 (s, 1H), 8.27 (bs, 1H), 8.13 (s, 1H), 7.83-7.79 (m, 1H), 7.37-7.34 (d, J=12 Hz, 1H), 7.31-7.29 (d, J=12 Hz, 1H), 7.23 (s, 1H), 7.04 (m, 1H), 3.82 (s, 1H), 3.72 (s, 3H), 3.35 (s, 3H), 3.25-3.19 (m, 1H), 2.82-2.81 (m, 2H), 2.66-2.56 (m, 2H), 1.34-1.26 (m, 2H), 1.08-1.01 (m, 2H), 0.90 (s, 3H), 0.48-0.42 (t, J=12 Hz, 2H).

### Example I-83: 1-((1s,4s)-4-hydroxy-4-methylcyclohexyl)-3-methyl-8-(1-methyl-1H-pyrazol-4-yl)-9-(pyrazolo[1,5-a]pyridin-5-yl)-1,3-dihydro-2H-imidazo[4,5-d]pyrrolo[1,2-b]pyridazin-2-one

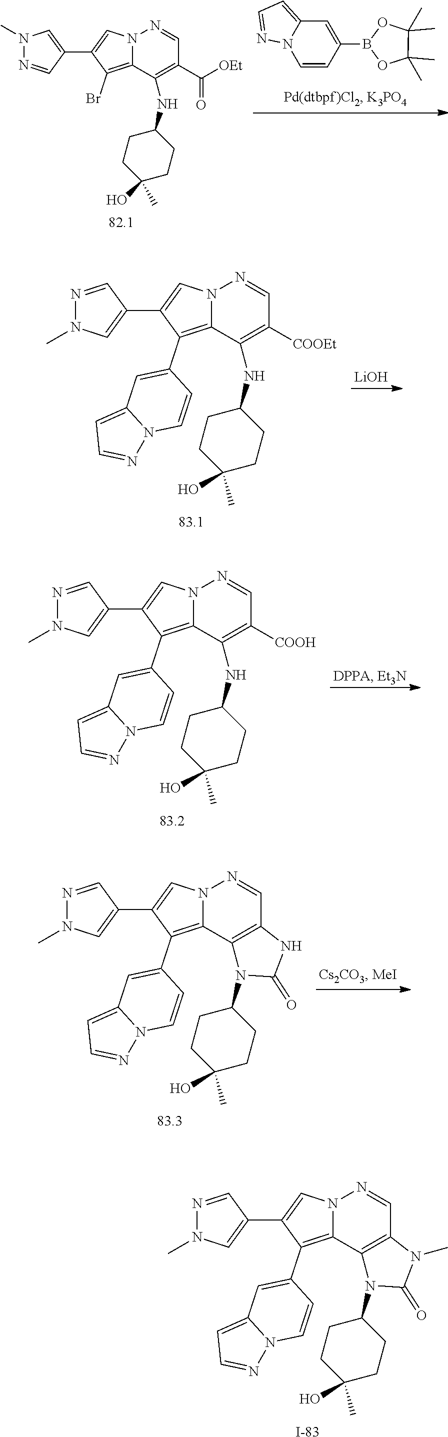

Synthesis of compound 83.1. Compound 83.1 was prepared from compound 82.1 and 5-(4,4,5,5-tetramethyl-1,3,2-dioxaborolan-2-yl)pyrazolo[1,5-a]pyridine following the procedure described in the synthesis of compound 59.12. The product was purified by flash column chromatography on silica gel (Combiflash®, 63% ethyl acetate in hexane). MS(ES): m/z 514.3 [M+H]⁺.

Synthesis of compound 83.2. Compound 83.2 was prepared from compound 83.1 following the procedure described in the synthesis of compound 1.10. The product was triturated with pentane, dried under vacuum and used in the next step without purification. MS(ES): m/z 486.5 [M+H]⁺.

Synthesis of compound 83.3. Compound 83.3 was prepared from compound 83.2 following the procedure described in the synthesis of compound 1.11. The product was purified by flash column chromatography on silica gel (Combiflash®, 3.5% methanol in DCM). MS(ES): m/z 483.5 [M+H]⁺.

Synthesis of compound I-83. Compound I-83 was prepared from compound 83.3 following the procedure described in the synthesis of compound 1.12. The product was purified by flash column chromatography on silica gel (2.8% methanol in DCM. MS(ES): m/z 497.2 [M+H]⁺, ¹H-NMR (400 MHz, DMSO-d₆): δ 8.80 (d, J=6.8 Hz, 1H), 8.42 (s, 1H), 8.13-8.09 (m, 2H), 7.73 (s, 1H), 7.42 (s, 1H), 7.16 (s, 1H), 6.86 (d, J=7.2 Hz, 1H), 6.65 (s, 1H), 3.76 (s, 1H), 3.70 (s, 3H), 3.45 (s, 1H), 3.39 (s, 3H), 1.33-1.23 (m, 2H), 1.11-1.09 (m, 1H), 1.02-0.99 (m, 2H), 0.61 (s, 3H), 0.23 (t, J=14.0 Hz, 1H), (s, 1H), −0.27-−0.33 (m, 1H).

### Example I-84: 9-(3,4-dihydro-2H-benzo[b][1,4]oxazin-6-yl)-1-((1s,4s)-4-hydroxy-4-methylcyclohexyl)-3-methyl-8-(1-methyl-1H-pyrazol-4-yl)-1,3-dihydro-2H-imidazo[4,5-d]pyrrolo[1,2-b]pyridazin-2-one

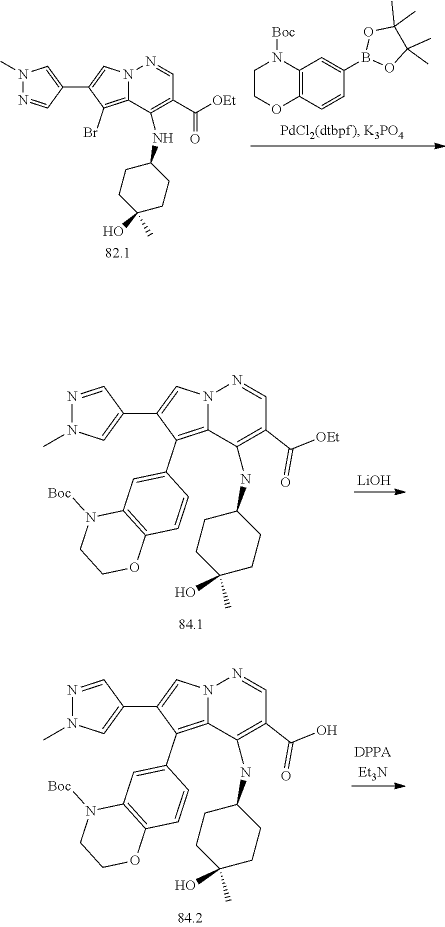

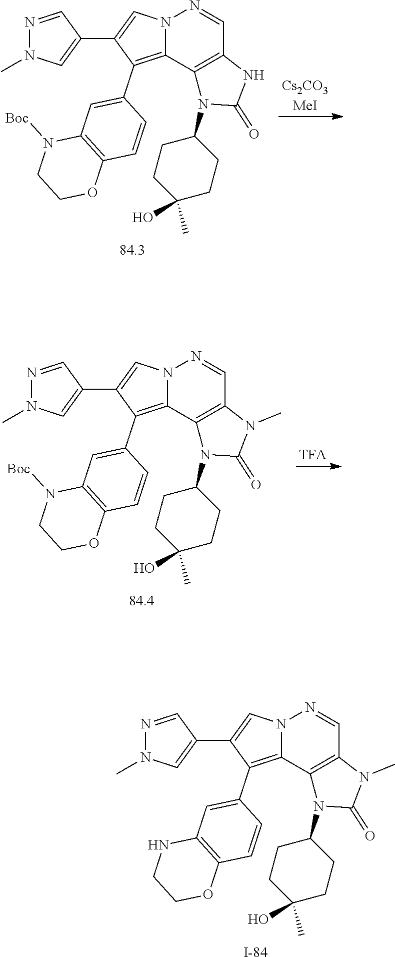

Synthesis of compound 84.1. Compound 84.1 was prepared from compound 82.1 and tert-butyl 6-(4,4,5,5-tetramethyl-1,3,2-dioxaborolan-2-yl)-2,3-dihydro-4H-benzo[b][1,4]oxazine-4-carboxylate following the procedure described in the synthesis of compound 59.12. The product was purified by flash column chromatography on silica gel (Combiflash®, 18% ethyl acetate in hexane). MS(ES): m/z 631.8 [M+H]⁺.

Synthesis of compound 84.2. Compound 84.2 was prepared from compound 84.1 following the procedure described in the synthesis of compound 1.10. The product was triturated with pentane, dried under vacuum and used in the next step without purification. MS(ES): m/z 603.7 [M+H]⁺.

Synthesis of compound 84.3. Compound 84.3 was prepared from compound 84.2 following the procedure described in the synthesis of compound 1.11. The product was purified by flash column chromatography on silica gel (Combiflash®, 3.0% methanol in DCM). MS(ES): m/z 600.2 [M+H]⁺.

Synthesis of compound 84.4. Compound 84.4 was prepared from compound 84.3 following the procedure described in the synthesis of compound 1.12. The product was purified by flash column chromatography on silica gel (2.8% methanol in DCM). MS(ES): m/z 614.5 [M+H]⁺.

Synthesis of compound I-84. Compound I-84 was prepared from compound 84.4 following the procedure described in the synthesis of compound 1.14. The product was purified by flash column chromatography on silica gel (3.8% methanol in DCM). MS(ES): m/z 514.41 [M+H]⁺, ¹H-NMR (400 MHz, DMSO-d₆): δ 8.32 (s, 1H), 8.07 (s, 1H), 7.24 (s, 1H), 7.05 (s, 1H), 6.75 (d, J=8.0 Hz, 1H), 6.48 (d, J=2.0 Hz, 1H), 6.43 (d, J=8.0 Hz, 1H), 5.93 (s, 1H), 4.21 (m, 2H), 3.85 (m, 1H), 3.72 (s, 3H), 3.65-3.62 (m, 1H), 3.30 (s, 3H), 2.64-2.61 (m, 2H), 1.39-1.35 (m, 2H), 1.32-1.30 (m, 2H), 1.11-1.07 (m, 2H), 1.01 (s, 3H), 0.85-0.78 (m, 2H).

### Example I-85: methyl ((1R,3R)-3-(9-(5-cyclopropyl-1H-pyrazol-3-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

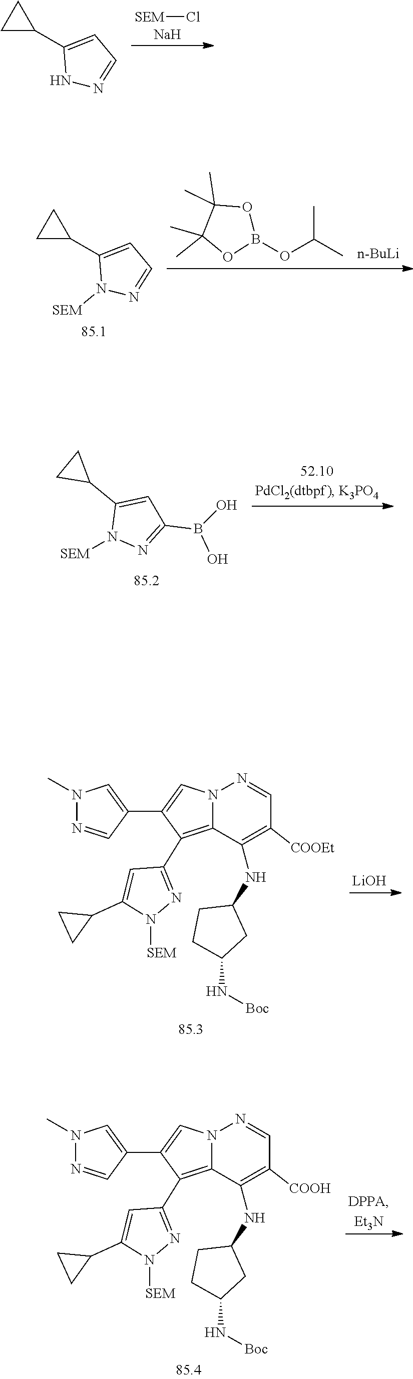

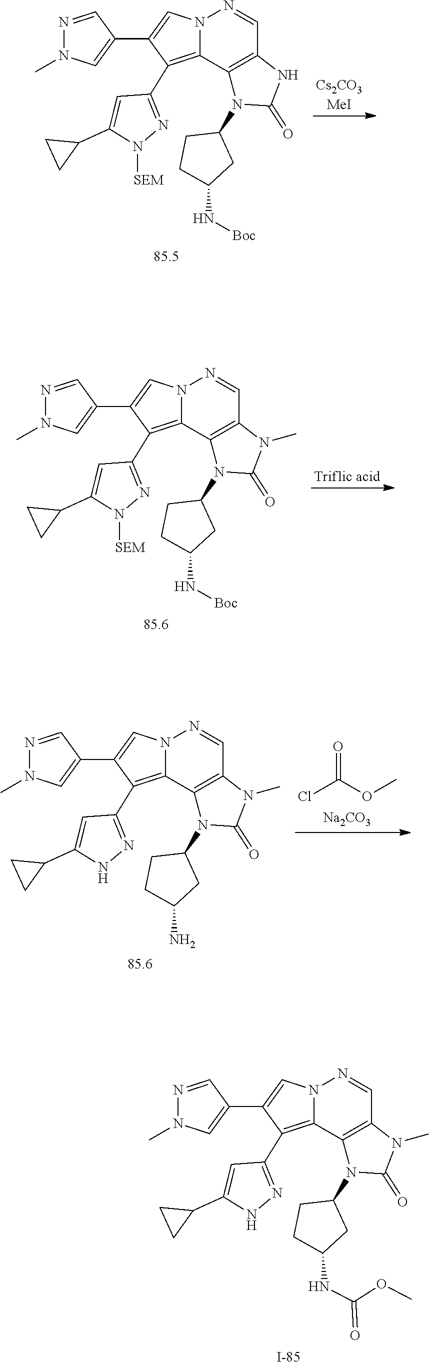

Synthesis of compound 85.1. To a solution of 5-cyclopropyl-1H-pyrazole (5 g, 0.046 mmol, 1.0 equiv) in THF (50 mL) was added sodium hydride (3.7 g, 0.092 mmol, 2.0 equiv) at 0° C. The reaction mixture was stirred at 0° C. for 15 min. 2-(trimethylsilyl) ethoxy methyl chloride (11.5 g, 0.069 mmol, 1.5 equiv) was added and stirred at room temperature for 2 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (50% ethyl acetate in hexane) to afford 85.1 (5 g, 45%). MS(ES): m/z 239.2 [M+H]⁺.

Synthesis of compound 85.2. To a solution of 85.1 (5 g, 0.021 mmol, 1.0 equiv) in THF (50 mL) was added n-butyllithium (2.5 M in hexane, 12.6 mL, 0.031 mmol, 1.5 equiv) at −78° C. and stirred for 1 h. To the mixture was added 2-isopropoxy-4,4,5,5-tetramethyl-1,3,2-dioxaborolane (5.8 g, 0.031 mmol, 2.0 equiv) in THF dropwise at −78° C. and stirred for 2 h. It was quenched with aqueous ammonium chloride solution and extracted with ethyl acetate. The combined organic layers were washed with brine, dried over anhydrous sodium sulfate, filtered and concentrated under reduced pressure. The residue was purified by preparative HPLC (column: SUNFIRE C18 (250*19) mm, 5 μm; elution: 0.1% formic acid in water: MeCN; flow rate: 5 mL/min) to afford 85.2 (1.00 g, 17%). MS(ES): m/z 283.3 [M+H]⁺.

Synthesis of compound 85.3. Compound 85.3 was prepared from compound 85.2 and 52.10 following the procedure described in the synthesis of compound 59.12. The product was purified by flash column chromatography on silica gel (50% ethyl acetate in hexane). MS(ES): m/z 705.6 [M+H]⁺.

Synthesis of compound 85.4. Compound 85.4 was prepared from compound 85.3 following the procedure described in the synthesis of compound 1.10. The product was triturated with pentane, dried under vacuum and used in the next step without purification. MS(ES): m/z 677.4 [M+H]⁺.

Synthesis of compound 85.5. Compound 85.5 was prepared from compound 85.4 following the procedure described in the synthesis of compound 1.11. The product was purified by flash column chromatography on silica gel (Combiflash®, 3.2% methanol in DCM). MS(ES): m/z 674.7 [M+H]⁺.

Synthesis of compound 85.6. Compound 85.6 was prepared from compound 85.5 following the procedure described in the synthesis of compound 1.12. The product was purified by flash column chromatography on silica gel (2.4% methanol in DCM). MS(ES): m/z 688.5 [M+H]⁺.

Synthesis of compound 85.7. Compound 85.7 was prepared from compound 85.6 following the procedure described in the synthesis of compound 21.8. The product was used in the next step without purification. MS(ES): m/z 458.3 [M+H]⁺.

Synthesis of I-85. Compound I-85 was prepared from compound 85.7 following the procedure described in the synthesis of compound I-1. The product was purified by flash column chromatography on silica gel (Combiflash®, 2.7% methanol in DCM). MS(ES): m/z 516.4 [M+H]⁺, ¹H-NMR (400 MHz, DMSO-d₆): δ 12.71 (s, 1H), 8.42 (s, 1H), 8.15 (s, 1H), 7.38 (s, 1H), 7.18 (s, 1H), 6.89 (s, 1H), 6.10 (bs, 1H), 4.11 (m, 1H), 3.75 (s, 3H), 3.51 (s, 3H), 3.36 (s, 3H), 2.16 (m, 1H), 1.97 (m, 3H), 1.59 (m, 1H), 1.37-1.28 (m, 1H), 1.21 (m, 1H), 1.04-0.90 (m, 3H), 0.66 (m, 2H).

### Example I-86: methyl ((1R,3R)-3-(3-methyl-9-(1-methyl-1H-indazol-5-yl)-8-(6-methylpyridazin-3-yl)-2-oxo-2,3-dihydro-1H-imidazo[4,5-d]pyrrolo[1,2-b]pyridazin-1-yl)cyclopentyl)carbamate

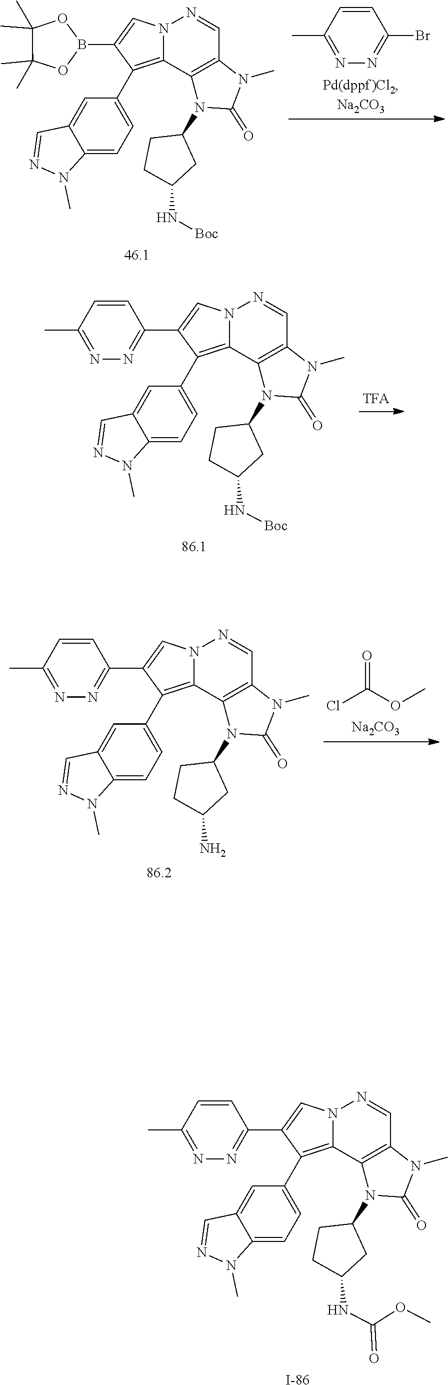

Synthesis of compound 86.1. Compound 86.1 was prepared from compound 46.1 and 3-bromo-6-methylpyridazine following the procedure described in the synthesis of compound 1.13. The product was purified by flash column chromatography on silica gel (2.4% methanol in DCM). MS(ES): m/z 594.4 [M+H]⁺.

Synthesis of compound 86.2. Compound 86.2 was prepared from compound 86.1 following the procedure described in the synthesis of compound 1.14. The product was used in the next step without purification. MS(ES): m/z 494.5 [M+H]⁺.

Synthesis of I-86. Compound I-86 was prepared from compound 86.2 following the procedure described in the synthesis of compound I-1. The product was purified by flash column chromatography on silica gel (Combiflash®, 2.9% methanol in DCM). MS(ES): m/z 552.28 [M+H]⁺, ¹H-NMR (400 MHz, DMSO-d₆): δ 8.53-8.41 (m, 2H), 8.03 (s, 1H), 7.78-7.72 (m, 2H), 7.44-7.42 (m, 1H), 7.26-7.24 (m, 1H), 7.07-6.93 (m, 1H), 6.72-6.36 (m, 1H), 4.12-4.11 (m, 3H), 3.87-3.66 (m, 2H), 3.48 (s, 3H), 3.45 (s, 3H), 3.37 (s, 3H), 1.99-1.99 (m, 1H), 1.86-1.74 (m, 1H), 1.49-1.36 (m, 1H), 1.23-0.77 (m, 2H), 0.35-−0.18 (m, 1H).

### Example I-87: 1-((1s,4s)-4-hydroxy-4-methylcyclohexyl)-3-methyl-8-(1-methyl-1H-pyrazol-4-yl)-9-(thieno[2,3-c]pyridin-2-yl)-1,3-dihydro-2H-imidazo[4,5-d]pyrrolo[1,2-b]pyridazin-2-one

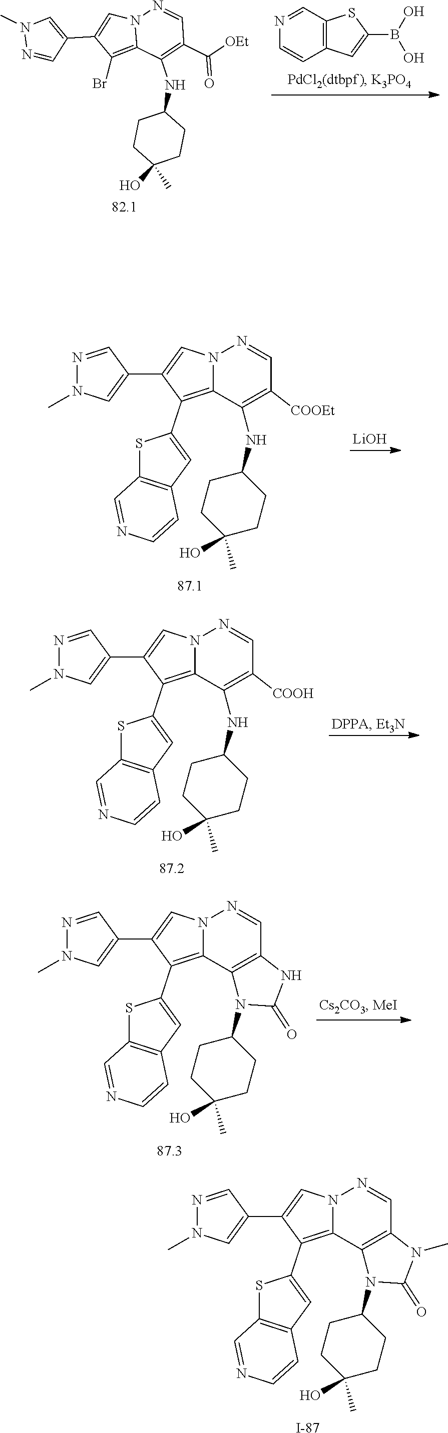

Synthesis of compound 87.1. Compound 87.1 was prepared from compound 82.1 and thieno[2,3-c]pyridin-2-ylboronic acid following the procedure described in the synthesis of compound 59.12. The product was purified by flash column chromatography on silica gel (Combiflash®, 30% ethyl acetate in hexane). MS(ES): m/z 531.65 [M+H]⁺.

Synthesis of compound 87.2. Compound 87.2 was prepared from compound 87.1 following the procedure described in the synthesis of compound 1.10. The product was triturated with pentane, dried under vacuum and used in the next step without purification. MS(ES): m/z 503.59 [M+H]⁺.

Synthesis of compound 87.3. Compound 87.3 was prepared from compound 87.2 following the procedure described in the synthesis of compound 1.11. The product was purified by flash column chromatography on silica gel (Combiflash®, 0-3% methanol in DCM). MS(ES): m/z 500.59 [M+H]⁺.

Synthesis of compound I-87. Compound I-87 was prepared from compound 87.3 following the procedure described in the synthesis of compound 1.12. The product was purified by flash column chromatography on silica gel (0-3% methanol in DCM). MS(ES): m/z 514.29 [M+H]⁺, ¹H NMR (DMSO-d₆, 400 MHz): δ 9.28 (s, 1H), 8.53 (m, 2H), 8.20 (s, 1H), 7.89 (s, 1H), 7.65 (s, 1H), 7.44 (s, 1H), 7.14 (s, 1H), 3.74 (s, 1H), 3.69 (s, 3H), 3.50 (2, 2H), 1.55 (m, 1H), 1.22 (s, 3H), 1.10 (bs, 5H), 0.85 (bs, 1H) 0.42 (s, 3H).

### Example I-88: methyl ((1R,3R)-3-(3-methyl-8-(1-methyl-1H-pyrazol-4-yl)-2-oxo-9-(2-oxo-2,3-dihydrobenzo[d]oxazol-5-yl)-2,3-dihydro-1H-imidazo[4,5-d]pyrrolo[1,2-b]pyridazin-1-yl)cyclopentyl)carbamate

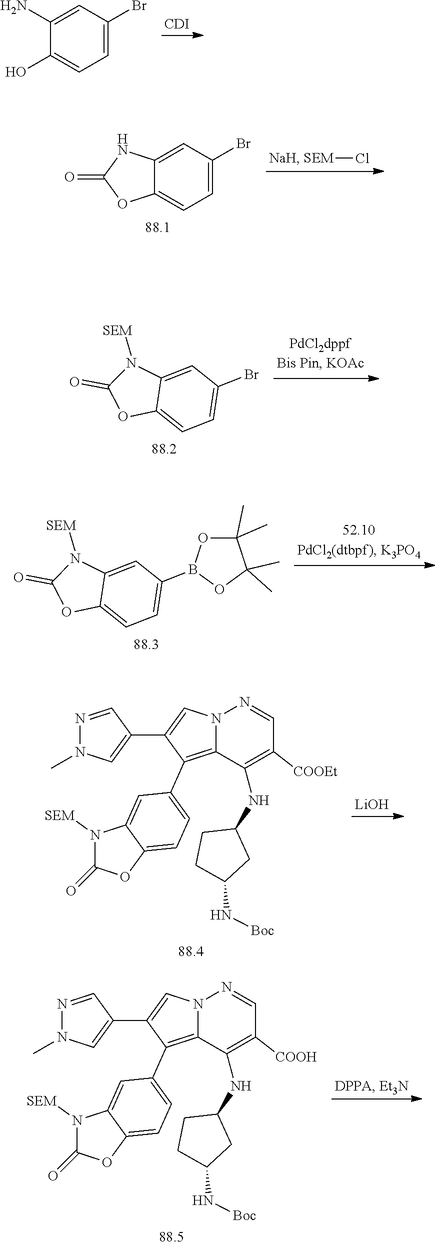

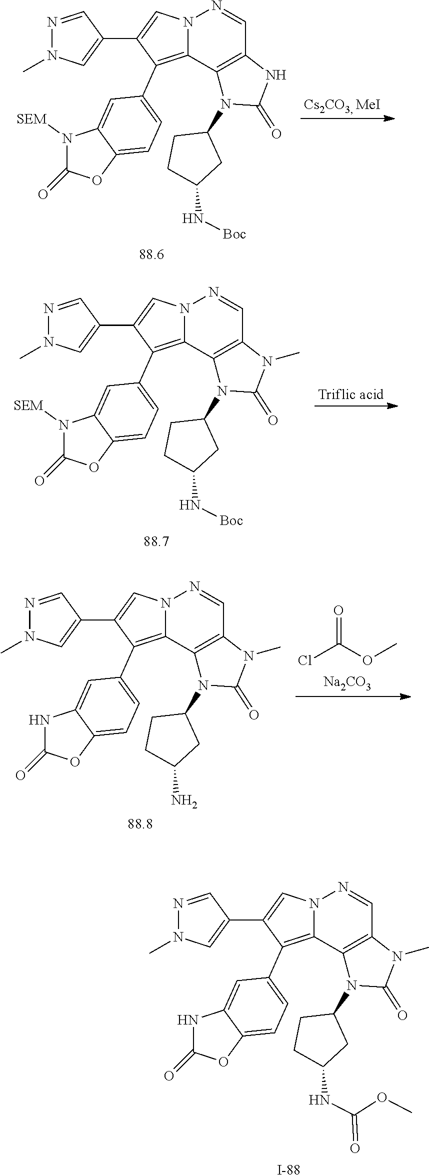

Synthesis of compound 88.1. To a solution of 2-amino-4-bromophenol (1 g, 5.3 mmol, 1.0 equiv) in THF (10 mL) was added 1,1-carbonyldiimidazole (1.03 g, 6.3 mmol, 1.2 equiv). The reaction mixture was stirred at room temperature for 2 h. It was transferred into water and extracted with ethyl acetate. The combined organic layers were washed with brine, dried over anhydrous sodium sulfate, filtered and concentrated under reduced pressure. The residue was purified by flash column chromatography on silica gel (Combiflash®, 21% ethyl acetate in hexane) to afford 88.1 (0.750 g, 66%). MS(ES): m/z 214 and 215 [M+H]⁺.

Synthesis of compound 88.2. To a solution of 88.1 (0.750 g, 3.5 mmol, 1.0 equiv) in DMF (8 mL) was added sodium hydride (60% dispersion in mineral oil, 0.336 g, 7.0 mmol, 2 equiv) in small portions at 0° C. and stirred for 30 min. To the suspension was added 2-(trimethylsilyl) ethoxymethyl chloride (0.876 g, 5.2 mmol, 1.5 equiv). The reaction mixture was stirred at 0° C. for 2 h. It was quenched with ice-water and extracted with ethyl acetate. The combined organic layers were washed with brine, dried over anhydrous sodium sulfate, filtered and concentrated under reduced pressure to afford 88.2 (0.740 g, 61%). 344.28 and 345.19 [M+H]⁺.

Synthesis of compound 88.3. A mixture of 88.2 (0.740 g, 2.15 mmol, 1.0 equiv), bis(pinacolato)diboron (0.818 g, 3.22 mmol, 1.5 equiv) and potassium acetate (0.631 g, 6.44 mmol, 3.0 equiv) in 1,4-dioxane (10 mL) was purged by bubbling through a stream of argon for 5 min. [1,1′-Bis(diphenylphosphino) ferrocene]dichloropalladium (II) (0.157 g, 0.214 mmol, 0.1 equiv) was added. The reaction mixture was stirred at 100° C. for 2 h. It was cooled to room temperature, transferred into water and extracted with ethyl acetate. The combined organic layers were washed with brine, dried over anhydrous sodium sulfate, filtered and concentrated under reduced pressure. The residue was purified by flash column chromatography on silica gel (Combiflash®, 24% ethyl acetate in hexane) to afford 88.3 (0.478 g, 57%). MS(ES): m/z 392.3 [M+H]⁺.

Synthesis of compound 88.4. Compound 88.4 was prepared from compound 88.3 and 52.10 following the procedure described in the synthesis of compound 59.12. The product was purified by flash column chromatography on silica gel (2.1% methanol in DCM). MS(ES): m/z 732.8 [M+H]⁺.

Synthesis of compound 88.5. Compound 88.5 was prepared from compound 88.4 following the procedure described in the synthesis of compound 1.10. The product was triturated with pentane, dried under vacuum and used in the next step without purification. MS(ES): m/z 704.6 [M+H]⁺.

Synthesis of compound 88.6. Compound 88.6 was prepared from compound 88.5 following the procedure described in the synthesis of compound 1.11. The product was purified by flash column chromatography on silica gel (Combiflash®, 2.4% methanol in DCM). MS(ES): m/z 701.6 [M+H]⁺.

Synthesis of compound 88.7. Compound 88.7 was prepared from compound 88.6 following the procedure described in the synthesis of compound 1.12. The product was purified by flash column chromatography on silica gel (2.1% methanol in DCM). MS(ES): m/z 715.7

Synthesis of compound 88.8. Compound 88.8 was prepared from compound 88.7 following the procedure described in the synthesis of compound 21.8. The product was used in the next step without purification. MS(ES): m/z 484.52 [M+H]⁺.

Synthesis of compound I-88. Compound I-88 was prepared from compound 88.8 following the procedure described in the synthesis of compound I-1. The product was purified by flash column chromatography on silica gel (Combiflash®, 3.4% methanol in DCM). MS (ES): m/z 543.4 [M+H]⁺, ¹H-NMR (400 MHz, DMSO-d₆): δ 13.70 (s, 1H), 8.44 (s, 1H), 8.26 (s, 1H), 8.22 (s, 1H), 8.20 (s, 1H), 7.73 (d, J=4.0 Hz, 1H), 7.32 (s, 1H), 7.13 (d, J=8.0 Hz, 1H), 6.70 (d, J=6.0 Hz, 1H), 3.93 (m, 1H), 3.52 (s, 3H), 3.47-3.43 (m, 1H), 3.40-3.39 (m, 1H), 3.36-3.35 (m, 1H), 3.32 (s, 3H), 2.03-2.02 (m, 1H), 1.80-1.71 (m, 1H), 1.54 (m, 1H), 1.10 (s, 3H), 0.45 (m, 0.5H), 0.10 (m, 0.5H).

### Example I-89: methyl ((1R,3R)-3-(8-(3,6-dihydro-2H-pyran-4-yl)-9-(3-fluoro-4-(methylcarbamoyl)phenyl)-3-methyl-2-oxo-2,3-dihydro-1H-imidazo[4,5-d]pyrrolo[1,2-b]pyridazin-1-yl)cyclopentyl)carbamate

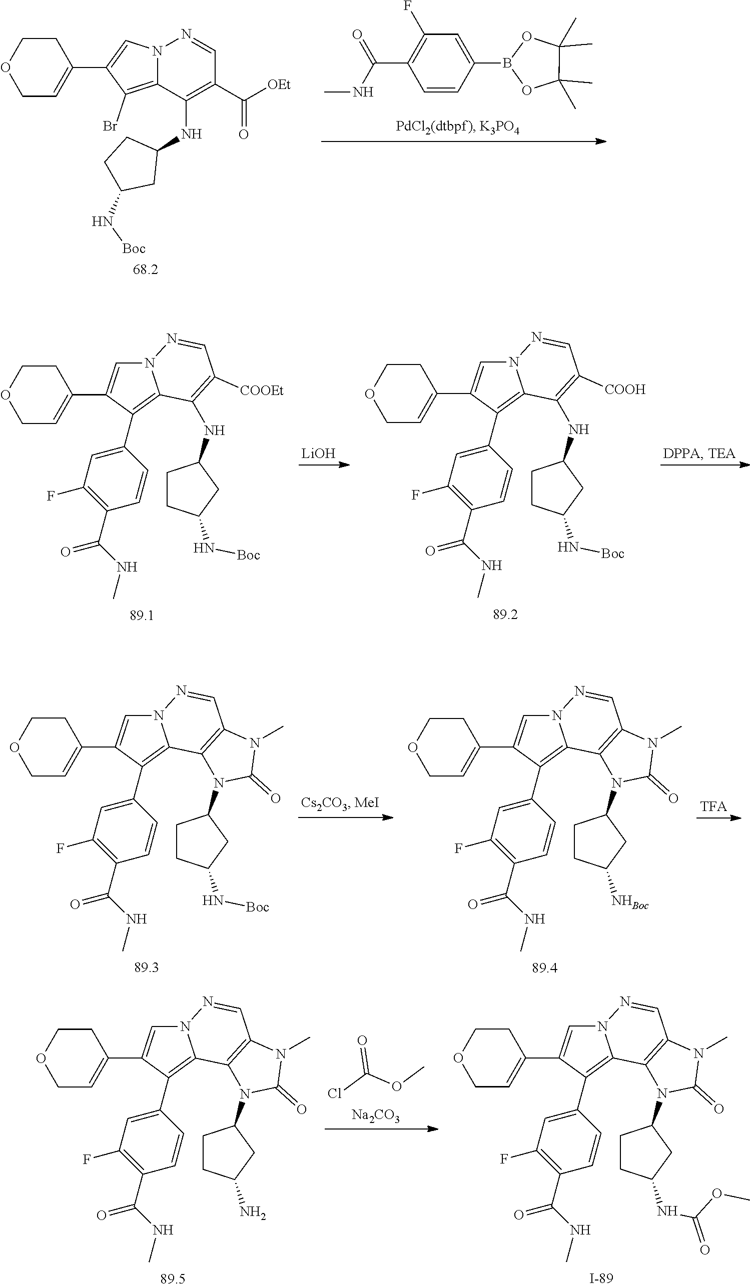

Synthesis of compound 89.1. Compound 89.1 was prepared from compound 68.2 and 2-fluoro-N-methyl-4-(4,4,5,5-tetramethyl-1,3,2-dioxaborolan-2-yl)benzamide following the procedure described in the synthesis of compound 59.12. The product was purified by flash column chromatography on silica gel (40% ethyl acetate in hexane). MS(ES): m/z 622 [M+H]⁺.

Synthesis of compound 89.2. Compound 89.2 was prepared from compound 89.1 following the procedure described in the synthesis of compound 1.10. The product was triturated with pentane, dried under vacuum and used in the next step without purification. MS(ES): m/z 594.3 [M+H]⁺.

Synthesis of compound 89.3. Compound 89.3 was prepared from compound 89.2 following the procedure described in the synthesis of compound 1.11. The product was purified by flash column chromatography on silica gel (Combiflash®, 3.2% methanol in DCM). MS(ES): m/z 591.5 [M+H]⁺.

Synthesis of compound 89.4. Compound 89.4 was prepared from compound 89.3 following the procedure described in the synthesis of compound 1.12. The product was purified by flash column chromatography on silica gel (1.6% methanol in DCM). MS(ES): m/z 605.3 [M+H]⁺.

Synthesis of compound 89.5. Compound 89.5 was prepared from compound 89.4 following the procedure described in the synthesis of compound 1.14. The product was used in the next step without purification. MS(ES): m/z 505.6 [M+H]⁺.

Synthesis of I-89. Compound I-89 was prepared from compound 89.5 following the procedure described in the synthesis of compound I-1. The product was purified by flash column chromatography on silica gel (Combiflash®, 3.3% methanol in DCM). MS(ES): m/z 563.36 [M+H]⁺, ¹H-NMR (400 MHz, DMSO-d₆): δ 8.44 (s, 1H), 8.34 (s, 1H), 7.96 (s, 1H), 7.73 (t, J=16 Hz, 1H), 7.36-7.30 (m, 2H), 6.80 (s, 1H), 5.42 (s, 1H), 4.01-3.97 (m, 2H), 3.66 (m, 2H), 3.49 (s, 3H), 3.35 (s, 3H), 2.82 (d, J=4 Hz, 3H), 2.18 (m, 2H), 2.09-1.97 (m, 2H), 1.81 (m, 1H), 1.39 (m, 1H), 1.23-1.15 (m, 2H), 0.73 (m, 2H).

### Example I-90: methyl ((1R,3R)-3-(9-(5-cyclopropyl-6-oxo-1,6-dihydropyridazin-3-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

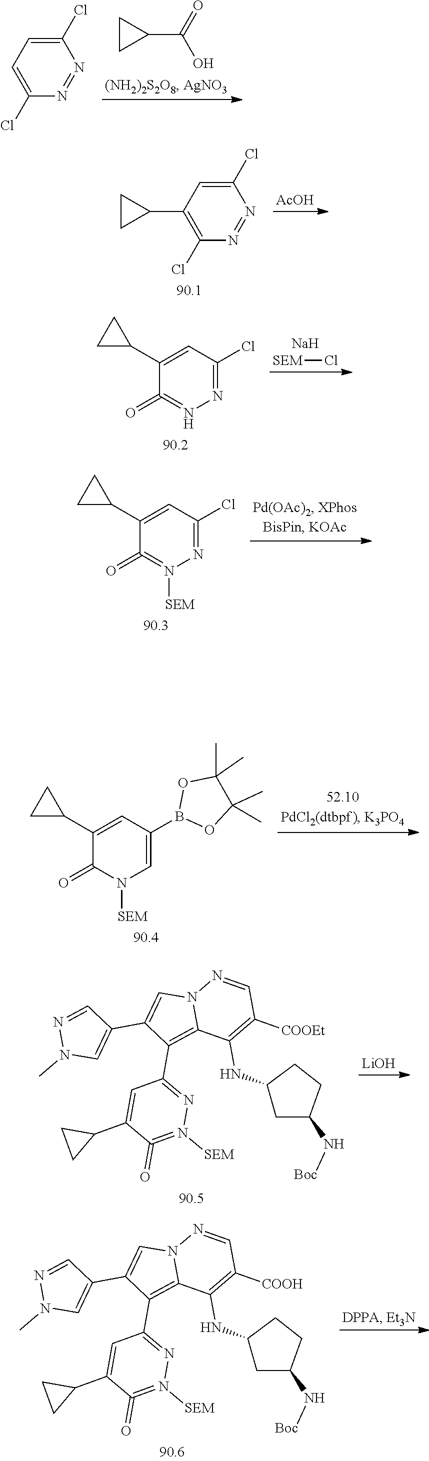

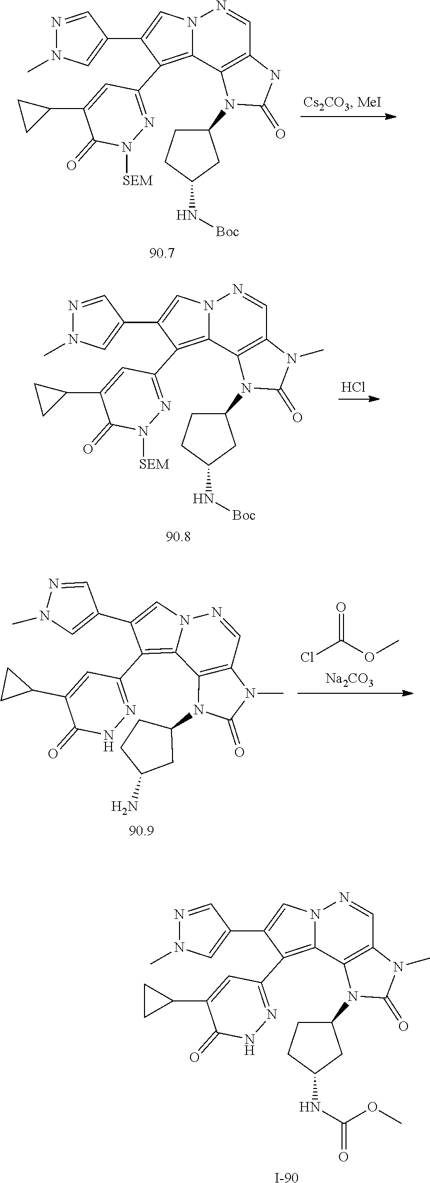

Synthesis of compound 90.1. To a solution of 3,6-dichloropyridazine (10 g, 67.13 mmol, 1.0 equiv) in sulfuric acid (6 mL, 100.69 mmol, 1.5 equiv) and water (330 mL) was added cyclopropanecarboxylic acid (9.23 g, 107.40 mmol, 1.6 equiv) and silver nitrate (1.13 g, 6.70 mmol, 0.1 equiv). The reaction mixture was stirred at 72° C. for 5 min and was added ammonium persulphate (22.9 g, 100.1 mmol, 1.5 equiv). The reaction mixture was stirred at 72° C. for 20 min. The reaction was cooled and quenched with 1 M NaOH solution (100 mL) and extract with ethyl acetate. The combined organic layers were dried over anhydrous sodium sulfate, filtered and concentrated under reduced pressure. The residue was purified by flash column chromatography on silica gel (Combiflash®, 20% ethyl acetate in hexane) to afford 90.1 (3 g, 24%). MS(ES): m/z 189.04 [M+H]⁺.

Synthesis of compound 90.2. To a stirred solution of 90.1 in acetic acid (30 mL) was refluxed for 16 h. The reaction mixture was cooled and quenched with saturated solution of sodium bicarbonate and extracted with ethyl acetate. The combined organic layers were dried over anhydrous sodium sulfate, filtered and concentrated under reduced pressure. The residue was purified by flash column chromatography on silica gel (Combiflash®, 30% ethyl acetate in hexane) to afford 90.2 (1.9 g, 70%). MS(ES): m/z 171.60 [M+H]⁺.

Synthesis of compound 90.3. To a solution of 90.2 (1.9 g, 11.1 mmol, 1.0 equiv) in DMF (20 mL) was added sodium hydride (60% dispersion in mineral oil, 1.06 g, 22.2 mmol, 2.0 equiv) in small portions at 0° C. and stirred for 30 min. To the suspension was added 2-(trimethylsilyl) ethoxymethyl chloride (2.78 g, 16.7 mmol, 1.5 equiv). The reaction mixture was stirred at 0° C. for 2 h. It was quenched with ice-water and extract with ethyl acetate. The combined organic layers were washed with brine, dried over anhydrous sodium sulfate, filtered and concentrated under reduced pressure. The residue was purified by flash column chromatography on silica gel (Combiflash®, 50% ethyl acetate in hexane) to afford 90.3 (1.6 g, 48%). MS(ES): m/z 301.86 [M+H]⁺.

Synthesis of compound 90.4. A mixture of 90.3 (1.6 g, 5.318 mmol, 1.0 equiv), bis(pinacolato)diboron (2.02 g, 7.977 mmol, 1.5 equiv) and potassium acetate (1.56 g, 15.95 mmol, 3.0 equiv) in 1,4-dioxane (16 mL) was purged by bubbling through a stream of argon for 5 min. [1,1′-Bis(diphenylphosphino) ferrocene]dichloropalladium (II) (0.38 g, 0.531 mmol, 0.1 equiv) was added. The reaction mixture was stirred at 100° C. for 2 h. It was cooled to room temperature, transferred into water and extracted with ethyl acetate. The combined organic layers were washed with brine, dried over anhydrous sodium sulfate, filtered and concentrated under reduced pressure. The residue was purified by flash column chromatography on silica gel (Combiflash®, 50% ethyl acetate in hexane) to afford 90.4 (1.2 g, 58%). MS(ES): m/z 393.38 [M+H]⁺.

Synthesis of compound 90.5. Compound 90.5 was prepared from compound 90.4 and 52.10 following the procedure described in the synthesis of compound 59.12. The product was purified by flash column chromatography on silica gel (5% methanol in DCM). MS(ES): m/z 733.96 [M+H]⁺.

Synthesis of compound 90.6. Compound 90.6 was prepared from compound 90.5 following the procedure described in the synthesis of compound 1.10. The product was triturated with pentane, dried under vacuum and used in the next step without purification. MS(ES): m/z 705.90 [M+H]⁺.

Synthesis of compound 90.7. Compound 90.7 was prepared from compound 90.6 following the procedure described in the synthesis of compound 1.11. The product was purified by flash column chromatography on silica gel (Combiflash®, 5% methanol in DCM). MS(ES): m/z 702.90 [M+H]⁺.

Synthesis of compound 90.8. Compound 90.8 was prepared from compound 90.7 following the procedure described in the synthesis of compound 1.12. The product was purified by flash column chromatography on silica gel (3.0% methanol in DCM). MS(ES): m/z 716.93 [M+H]⁺.

Synthesis of compound 90.9. To a solution of 90.8 (0.045 g) in DCM (3 mL) was added a 2 M solution of HCl in diethyl ether (1.0 mL) at 0° C. dropwise. The reaction mixture was stirred at room temperature for 1 h. The reaction mixture was concentrated under reduced pressure. The residue was triturated with n-pentane and dried under reduced pressure. It was used in the next step without purification. (0.022 g, 72%). MS(ES): m/z 486.55 [M+H]⁺.

Synthesis of compound I-90. Compound I-90 was prepared from compound 90.9 following the procedure described in the synthesis of compound I-1. The product was purified by flash column chromatography on silica gel (Combiflash®, 4% methanol in DCM). MS (ES): m/z 544.59 [M+H]⁺, ¹H-NMR (400 MHz, DMSO-d₆): δ 12.75 (s, 1H), 8.45 (s, 1H), 8.18 (s, 1H), 7.40 (s, 2H), 7.20 (s, 1H), 6.88 (s, 1H), 6.12 (s, 1H), 4.26 (m, 1H), 3.77 (s, 3H), 3.52 (s, 3H), 3.28 (s, 3H), 2.34 (m, 1H), 1.98 (m, 3H), 1.58 (m, 1H), 1.45 (m, 1H), 1.24 (m, 1H), 1.23-0.90 (m, 3H), 0.89 (m, 1H).

### Example I-91: methyl ((1R,3R)-3-(9-(5-cyclopropyl-1H-pyrazol-3-yl)-8-(3,6-dihydro-2H-pyran-4-yl)-3-methyl-2-oxo-2,3-dihydro-1H-imidazo[4,5-d]pyrrolo[1,2-b]pyridazin-1-yl)cyclopentyl)carbamate

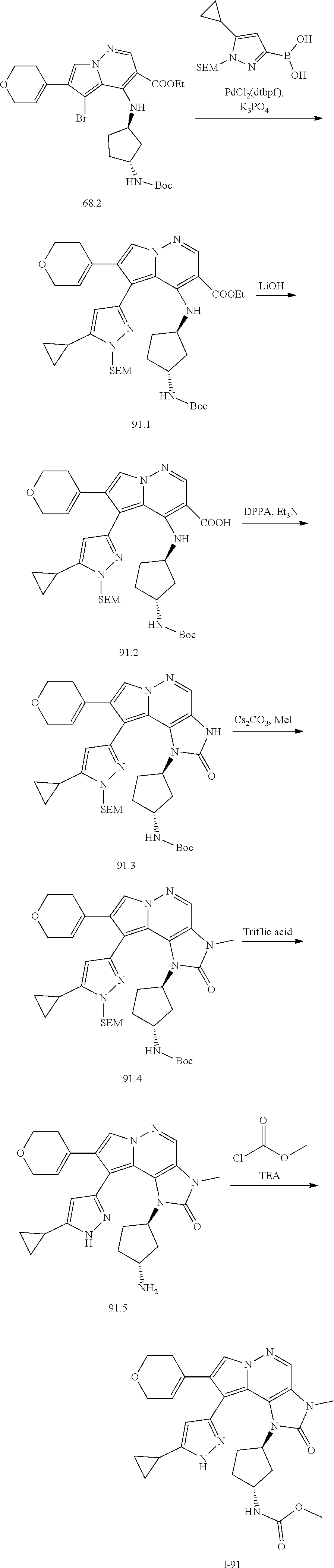

Synthesis of compound 91.1. Compound 91.1 was prepared from compound 68.2 and (5-cyclopropyl-1-((2-(trimethylsilyl)ethoxy)methyl)-1H-pyrazol-3-yl)boronic acid following the procedure described in the synthesis of compound 59.12. The product was purified by flash column chromatography on silica gel (50% ethyl acetate in hexane). MS(ES): m/z 707.28 [M+H]⁺.

Synthesis of compound 91.2. Compound 91.2 was prepared from compound 91.1 following the procedure described in the synthesis of compound 1.10. The product was triturated with pentane, dried under vacuum and used in the next step without purification. MS(ES): m/z 679.5 [M+H]⁺.

Synthesis of compound 91.3. Compound 91.3 was prepared from compound 91.2 following the procedure described in the synthesis of compound 1.11. The product was purified by flash column chromatography on silica gel (Combiflash®, 1.8% methanol in DCM). MS(ES): m/z 676.4 [M+H]⁺.

Synthesis of compound 91.4. Compound 91.4 was prepared from compound 91.3 following the procedure described in the synthesis of compound 1.12. The product was purified by flash column chromatography on silica gel (1.4% methanol in DCM). MS(ES): m/z 690.40 [M+H]⁺.

Synthesis of compound 91.5. Compound 91.5 was prepared from compound 91.4 following the procedure described in the synthesis of compound 21.8. The product was used in the next step without purification. MS(ES): m/z 460.33 [M+H]⁺.

Synthesis of I-91. Compound I-91 was prepared from compound 91.5 following the procedure described in the synthesis of compound I-1. The product was purified by flash column chromatography on silica gel (Combiflash®, 3.7% methanol in DCM). MS(ES): m/z 518.3 [M+H]⁺, ¹H-NMR (400 MHz, DMSO-d₆): δ 12.45 (s, 1H), 8.35 (s, 1H), 7.87 (s, 1H), 6.38 (s, 1H), 6.04-5.98 (m, 1H), 5.57 (s, 1H), 4.14-4.10 (m, 1H), 4.03 (d, J=2.4 Hz, 2H), 3.70 (d, J=5.2 Hz, 2H), 3.55 (s, 3H), 3.37 (s, 3H), 2.33 (m, 3H), 2.01 (m, 3H), 1.60 (m, 2H), 1.41 (m, 1H), 1.12 (m, 1H), 0.90 (m, 2H), 0.78 (m, 2H).

### Example I-93: methyl ((1R,3R)-3-(9-(4,7-dihydro-5H-thieno[2,3-c]pyran-2-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

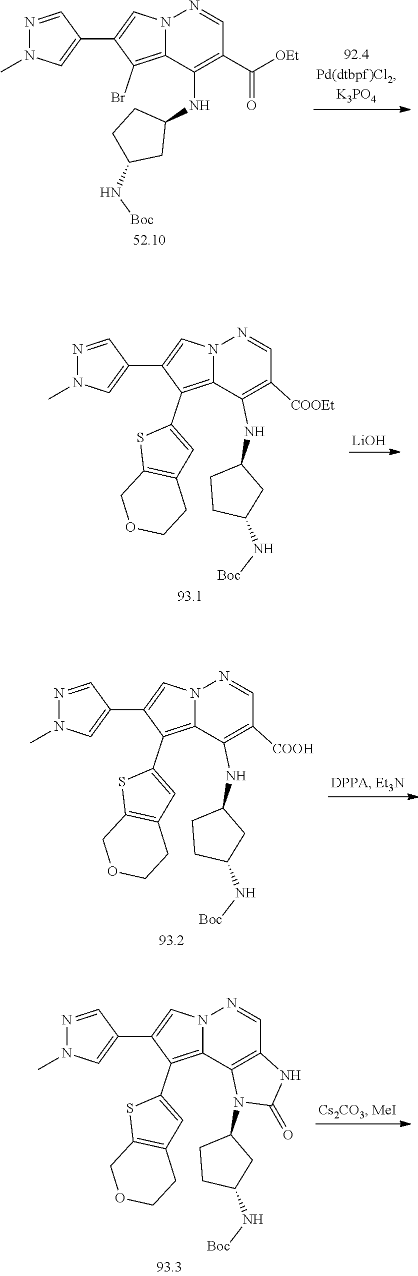

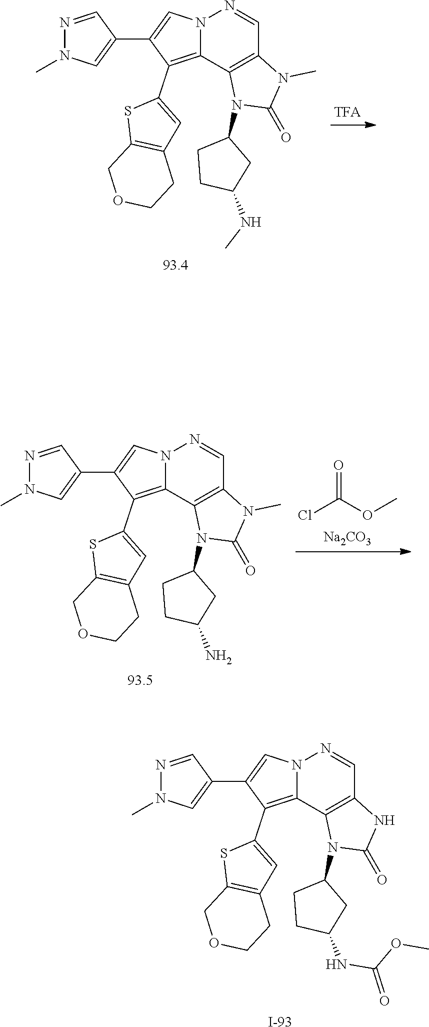

Synthesis of compound 93.1. Compound 93.1 was prepared from compound 92.4 and 52.10 following the procedure described in the synthesis of compound 59.12. The product was purified by flash column chromatography on silica gel (40% ethyl acetate in hexane). MS(ES): m/z 607.5 [M+H]⁺.

Synthesis of compound 93.2. Compound 93.2 was prepared from compound 93.1 following the procedure described in the synthesis of compound 1.10. The product was triturated with pentane, dried under vacuum and used in the next step without purification. MS(ES): m/z 579.5 [M+H]⁺.

Synthesis of compound 93.3. Compound 93.3 was prepared from compound 93.2 following the procedure described in the synthesis of compound 1.11. The product was purified by flash column chromatography on silica gel (Combiflash®, 3.2% methanol in DCM). MS(ES): m/z 576.7 [M+H]⁺.

Synthesis of compound 93.4. Compound 93.4 was prepared from compound 93.3 following the procedure described in the synthesis of compound 1.12. The product was purified by flash column chromatography on silica gel (1.4% methanol in DCM). MS(ES): m/z 590.7 [M+H]⁺.

Synthesis of compound 93.5. Compound 93.5 was prepared from compound 93.4 following the procedure described in the synthesis of compound 1.14. The product was used in next step without purification. MS(ES): m/z 490.6 [M+H]⁺.

Synthesis of I-93. Compound I-93 was prepared from compound 93.5 following the procedure described in the synthesis of compound I-1. The product was purified by flash column chromatography on silica gel (Combiflash®, 4.2% methanol in DCM). MS(ES): m/z 548.3 [M+H]⁺, ¹H NMR (DMSO-d₆, 400 MHz): δ 8.43 (s, 1H), 8.13 (s, 1H), 7.52 (s, 1H), 7.27 (s, 1H), 7.15-7.00 (m, 1H), 6.93 (s, 1H), 4.90-4.77 (m, 2H), 4.09-4.01 (m, 3H), 3.77 (s, 3H), 3.51 (s, 3H), 3.36 (s, 3H), 2.78 (bs, 2H), 2.14-2.08 (m, 1H), 2.08 (bs, 1H), 1.98-1.95 (bs, 2H), 1.58 (bs, 1H), 1.39 (bs, 1H), 0.96 (bs, 1H).

### Example I-94: 1-((1s,4s)-4-hydroxy-4-methylcyclohexyl)-9-(1H-indazol-3-yl)-3-methyl-8-(1-methyl-1H-pyrazol-4-yl)-1,3-dihydro-2H-imidazo[4,5-d]pyrrolo[1,2-b]pyridazin-2-one

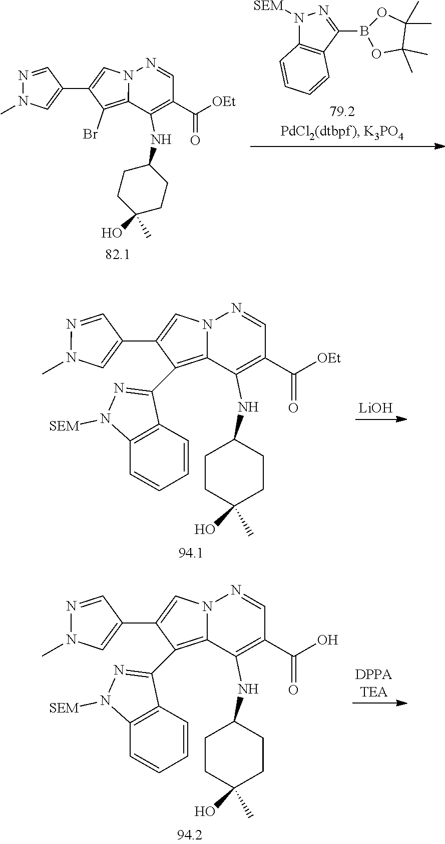 [SMILES: CCOC(=O)c1cnn2cc(-c3cnn(C)c3)c(Br)c2c1N[C@H]1CC[C@@](C)(O)CC1 ; CC1(C)OB(c2nn(COCC[Si](C)(C)C)c3ccccc23)OC1(C)C ; CCOC(=O)c1cnn2cc(-c3cnn(C)c3)c(-c3nn(COCC[Si](C)(C)C)c4ccccc34)c2c1N[C@H]1CC[C@@](C)(O)CC1 ; "LiOH" ; Cn1cc(-c2cn3ncc(C(=O)O)c(N[C@H]4CC[C@@](C)(O)CC4)c3c2-c2nn(COCC[Si](C)(C)C)c3ccccc23)cn1 ; "DPPATEA"]

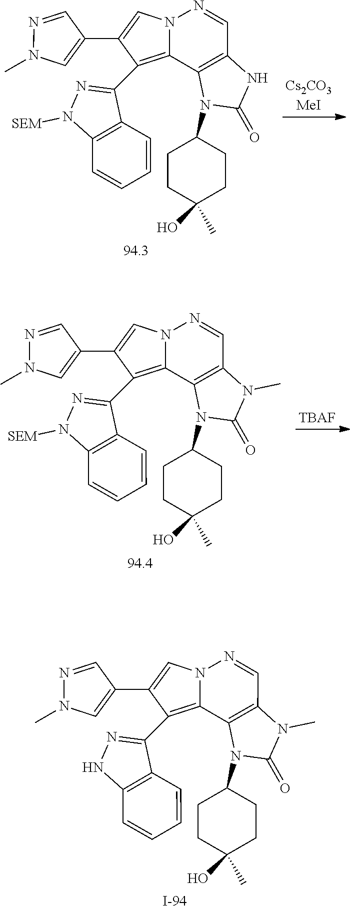

Synthesis of compound 94.1. Compound 94.1 was prepared from compound 82.1 and 79.2 following the procedure described in the synthesis of compound 59.12. The product was purified by flash column chromatography on silica gel (34% ethyl acetate in hexane). MS(ES): m/z 644.5 [M+H]⁺.

Synthesis of compound 94.2. Compound 94.2 was prepared from compound 94.1 following the procedure described in the synthesis of compound 1.10. The product was triturated with pentane, dried under vacuum and used in the next step without purification. MS(ES): m/z 616.2 [M+H]⁺.

Synthesis of compound 94.3. Compound 94.3 was prepared from compound 94.2 following the procedure described in the synthesis of compound 1.11. The product was purified by flash column chromatography on silica gel (Combiflash®, 1.4% methanol in DCM). MS(ES): m/z 613.6 [M+H]⁺.

Synthesis of compound 94.4. Compound 94.4 was prepared from compound 94.3 following the procedure described in the synthesis of compound 1.12. The product was purified by flash column chromatography on silica gel (1.2% methanol in DCM). MS(ES): m/z 627.4 [M+H]⁺.

Synthesis of I-94. To a solution of 94.4 (0.07 g, 0.11 mmol, 1.0 equiv) in THF (4.0 mL) was added tetra-n-butyl ammonium fluoride (0.291 g, 1.1 mmol, 10.0 equiv) dropwise at 0° C. and stirred at 70° C. for 3 h. It was quenched with water and extracted with ethyl acetate. The combined organic layers were washed with brine, dried over anhydrous sodium sulfate, filtered and concentrated under reduced pressure. The residue was purified by flash column chromatography on silica gel (Combiflash®, 3.4% methanol in DCM) to afford I-94 (0.011 g, 20%). MS(ES): m/z 497.4 [M+H]⁺, ¹H-NMR (400 MHz, DMSO-d₆): δ 13.45 (s, 1H), 8.45 (s, 1H), 8.23 (s, 1H), 7.63 (d, J=8.4 Hz, 1H), 7.42 (t, J=7.2 Hz, 1H), 7.29 (d, J=8 Hz, 1H), 7.25 (s, 1H), 7.06 (t, J=7.2 Hz, 1H), 6.85 (s, 1H), 3.67 (bs, 1H), 3.64 (s, 3H), 3.44-3.38 (m, 1H), 3.35 (s, 3H), 1.61-1.54 (m, 1H), 1.38-1.29 (m, 3H), 1.03-1.01 (m, 1H), 0.96-0.92 (m, 2H), 0.86-0.81 (m, 1H), 0.78 (s, 3H).

### Example I-95: (1R,2R)—N-((1s,4S)-4-(9-(1,3-dihydroisobenzofuran-5-yl)-3-methyl-8-(1-methyl-1H-pyrazol-4-yl)-2-oxo-2,3-dihydro-1H-imidazo[4,5-d]pyrrolo[1,2-b]pyridazin-1-yl)-1-methylcyclohexyl)-2-fluorocyclopropane-1-carboxamide

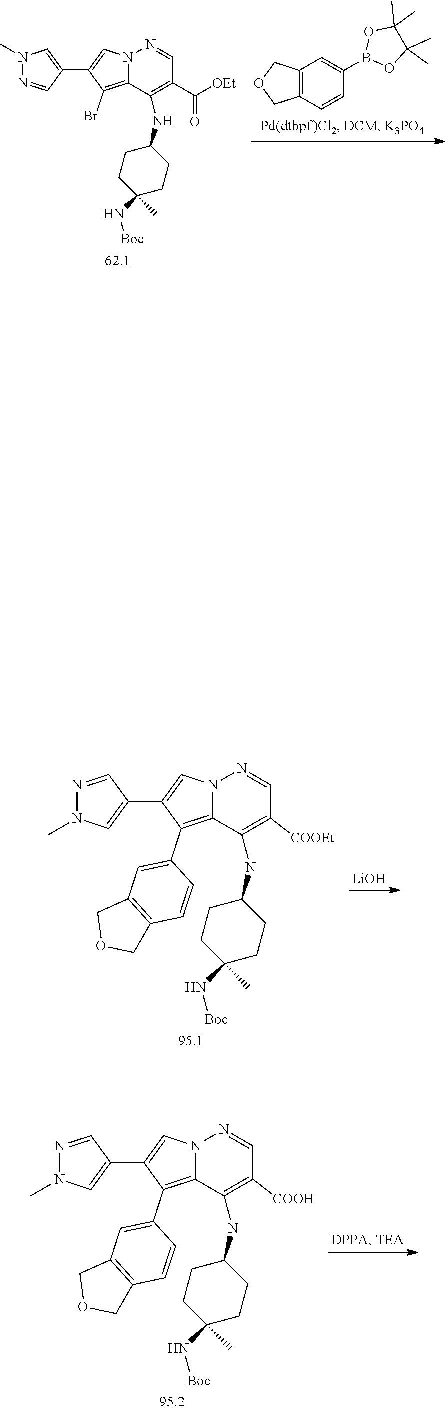

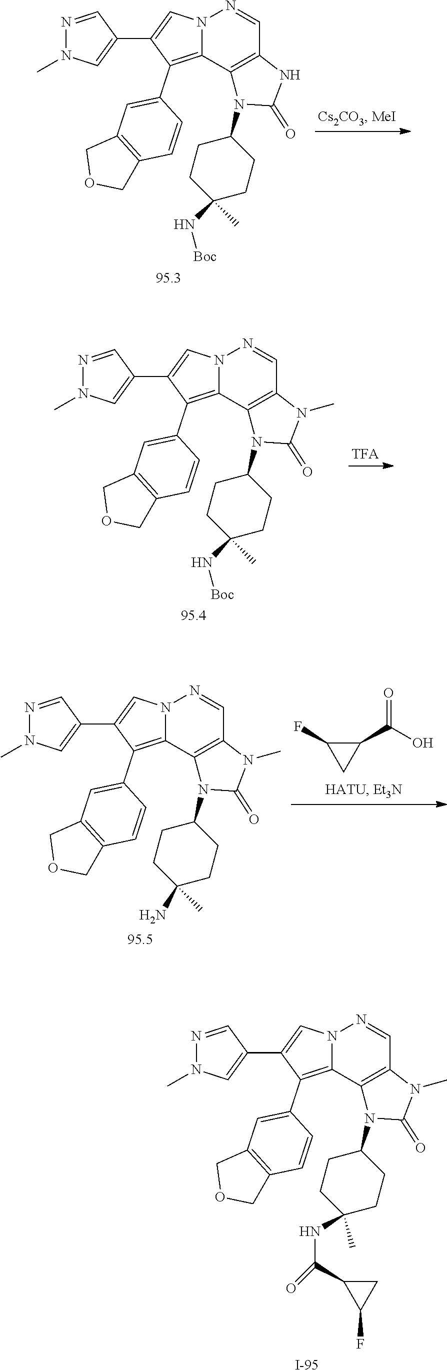

Synthesis of compound 95.1. Compound 95.1 was prepared from compound 62.1 and 2-(1,3-dihydroisobenzofuran-5-yl)-4,4,5,5-tetramethyl-1,3,2-dioxaborolane following the procedure described in the synthesis of compound 54.3. The product was purified by flash column chromatography on silica gel (42% ethyl acetate in n-hexane). MS (ES): m/z 615.7 [M+H]⁺.

Synthesis of compound 95.2. Compound 95.2 was prepared from compound 95.1 following the procedure described in the synthesis of compound 1.10. The product was purified by flash column chromatography on silica gel (42% ethyl acetate in n-hexane). MS (ES): m/z 615.7 [M+H]⁺.

Synthesis of compound 95.3. Compound 95.3 was prepared from compound 95.2 following the procedure described in the synthesis of compound 1.11. The product was purified by flash column chromatography on silica gel (2.5% methanol in DCM). MS (ES): m/z 584.6 [M+H]⁺.

Synthesis of compound 95.4. Compound 95.4 was prepared from compound 95.3 following the procedure described in the synthesis of compound 1.12. The product was purified by flash column chromatography on silica gel (2.8% methanol in DCM). MS (ES): m/z 598.7 [M+H]⁺.

Synthesis of compound 95.5. Compound 95.5 was prepared from compound 95.4 following the procedure described in the synthesis of compound 1.14. MS (ES): m/z 498.6 [M+H]⁺. The product was used in the next step without purification.

Synthesis of compound I-95. Compound I-95 was prepared from compound 95.4 and (1R,2R)-2-fluorocyclopropane-1-carboxylic acid following the procedure described in the synthesis of compound I-45. The product was purified by flash column chromatography on silica gel (3.6% methanol in DCM). MS (ES): m/z 584.4 [M+H]⁺, ¹H NMR (DMSO-d₆, 400 MHz): δ 8.38 (s, 1H), 8.12 (s, 1H), 7.48 (d, J=7.6 Hz, 1H), 7.41 (s, 1H), 7.35 (s, 1H), 7.30-7.28 (m, 1H), 7.26 (s, 1H), 6.97 (s, 1H), 5.22-4.95 (m, 4H), 4.90-4.60 (m, 1H), 3.71 (s, 3H), 3.39 (s, 3H), 2.09 (s, 1H), 1.98 (d, J=7.2 Hz, 1H), 1.82 (t, J=8.0 Hz, 2H), 1.47-1.44 (m, 2H), 1.34 (s, 1H), 1.23-1.22 (m, 2H), 1.04 (s, 3H), 0.90-0.80 (m, 2H), 0.28-0.22 (m, 1H).

### Example I-96: 9-(7-fluoro-1H-indazol-6-yl)-1-((1s,4s)-4-hydroxy-4-methylcyclohexyl)-3-methyl-8-(1-methyl-1H-pyrazol-4-yl)-1,3-dihydro-2H-imidazo[4,5-d]pyrrolo[1,2-b]pyridazin-2-one

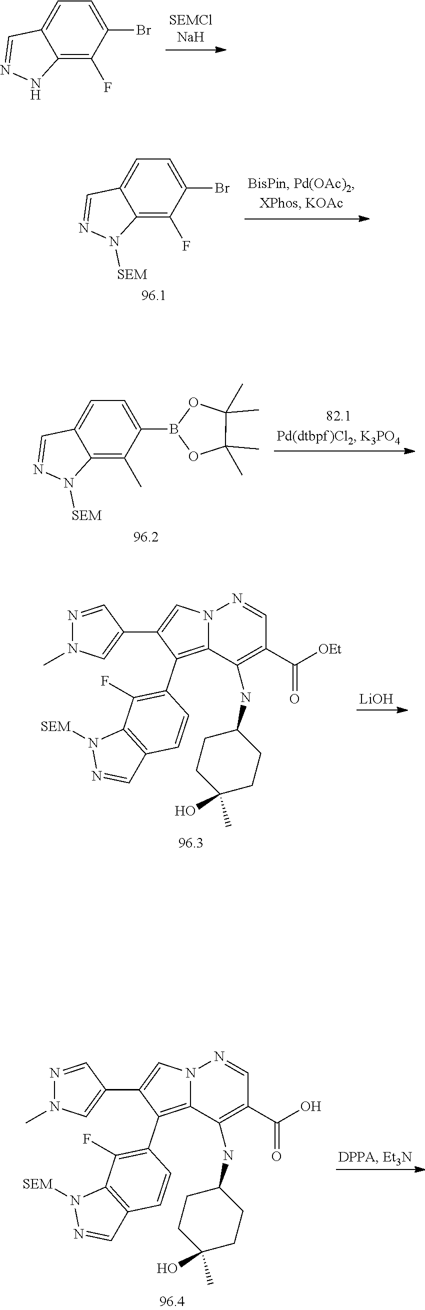

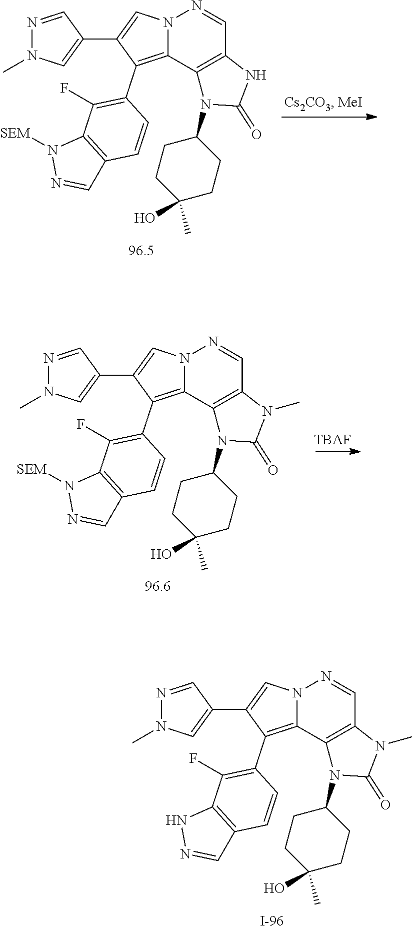

Synthesis of compound 96.1. Compound 96.1 was prepared from compound 6-bromo-7-fluoro-1H-indazole following the procedure described in the synthesis of compound 79.1. The product was purified by flash column chromatography on silica gel (Combiflash®, 8% ethyl acetate in hexane). MS(ES): m/z 345.2 and 347.2 [M+H]⁺.

Synthesis of compound 96.2. Compound 96.2 was prepared from compound 96.1 following the procedure described in the synthesis of compound 79.2. The product was purified by flash column chromatography on silica gel (10% ethyl acetate in hexane). MS(ES): m/z 393.2 [M+H]⁺.

Synthesis of compound 96.3. Compound 96.3 was prepared from compound 96.2 and 82.1 following the procedure described in the synthesis of compound 59.12. The product was purified by flash column chromatography on silica gel (Combiflash®, 65% ethyl acetate in hexane). MS(ES): m/z 662.6 [M+H]⁺.

Synthesis of compound 96.4. Compound 96.4 was prepared from compound 96.3 following the procedure described in the synthesis of compound 1.10. The product was triturated with pentane, dried under vacuum and used in the next step without purification. MS(ES): m/z 634.6 [M+H]⁺.

Synthesis of compound 96.5. Compound 96.5 was prepared from compound 96.4 following the procedure described in the synthesis of compound 1.11. The product was purified by flash column chromatography on silica gel (Combiflash®, 4.5% methanol in DCM). MS(ES): m/z 631.5 [M+H]⁺.

Synthesis of compound 96.6. Compound 96.6 was prepared from compound 96.5 following the procedure described in the synthesis of compound 1.12. The product was purified by flash column chromatography on silica gel (3.2% methanol in DCM. MS(ES): m/z 645.5 [M+H]⁺.

Synthesis of I-96. To a solution of 96.6 (0.060 g, 0.093 mmol, 1.0 equiv) in THF (3.0 mL) at 0° C. was added dropwise tetrabutylammonium fluoride (0.5 mL) and stirred for 30 min. It was quenched in ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 3.7% methanol in DCM) to afford I-96 (0.033 g, 69%). MS(ES): m/z 515.3 [M+H]⁺, ¹H NMR (DMSO-d₆, 400 MHz): δ 13.76 (s, 1H), 8.43 (s, 1H), 8.30 (s, 1H), 8.20 (s, 1H), 7.72 (d, J=8 Hz, 1H), 7.24 (s, 1H), 7.09-7.05 (t, J=8 Hz, 1H), 6.90 (s, 1H), 3.73 (s, 1H), 3.65 (s, 3H), 3.38 (s, 3H), 1.23 (s, 1H), 1.13-1.10 (m, 2H), 1.06-0.99 (m, 2H), 0.53 (m, 3H), 0.04-0.01 (m, 1H), −0.033-(−0.039) (m, 1H).

### Example I-97: (R)-2,2-difluoro-N-((1s,4S)-1-methyl-4-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclohexyl)cyclopropane-1-carboxamide

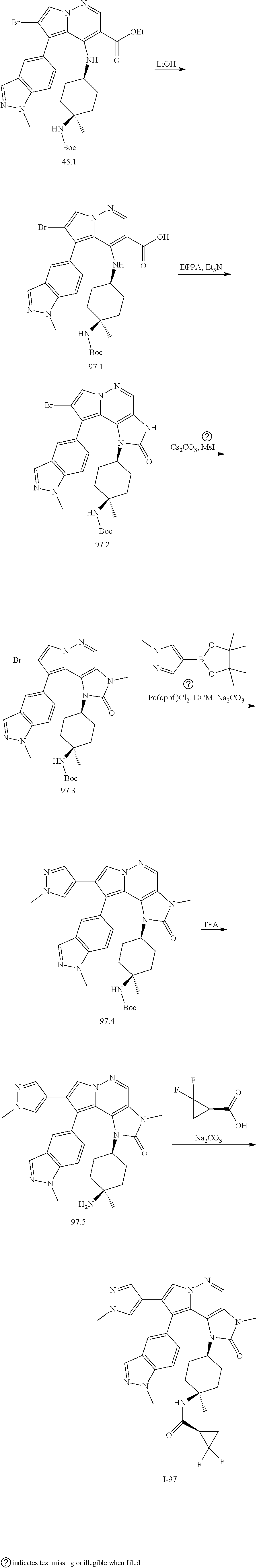

Synthesis of compound 97.1. Compound 97.1 was prepared from compound 45.1.

Compound 97.1 was prepared from compound 45.1 following the procedure described in the synthesis of compound 1.10. The product was triturated with pentane, dried under vacuum and used in the next step without purification. MS(ES): m/z 597.3 and 599.3 [M+H]⁺.

Synthesis of compound 97.2. Compound 97.2 was prepared from compound 97.1 following the procedure described in the synthesis of compound 1.11. The product was purified by flash column chromatography on silica gel (Combiflash®, 1.6% methanol in DCM). MS(ES): m/z 594.1 and 596.1 [M+H]⁺.

Synthesis of compound 97.3. Compound 97.3 was prepared from compound 97.2 following the procedure described in the synthesis of compound 1.12. The product was purified by flash column chromatography on silica gel (1.1% methanol in DCM). MS(ES): m/z 608.2 and 610.2 [M+H]⁺.

Synthesis of compound 97.4. Compound 97.4 was prepared from compound 97.3 following the procedure described in the synthesis of compound 1.13. The product was purified by flash column chromatography on silica gel (Combiflash®, 2.7% methanol in DCM). MS(ES): m/z 610.2 [M+H]⁺.

Synthesis of compound 97.5. Compound 97.5 was prepared from compound 97.4 following the procedure described in the synthesis of compound 1.14. The product was used in the next step without purification. MS(ES): m/z 510.4 [M+H]⁺.

Synthesis of I-97. Compound I-97 was prepared from compound 97.5 and (1R,2R)-2-fluorocyclopropane-1-carboxylic acid following the procedure described in the synthesis of compound I-45. The product was purified by flash column chromatography on silica gel (Combiflash®, 2.3% methanol in DCM). MS(ES): m/z 614.4 [M+H]⁺, ¹H NMR (DMSO-d₆, 400 MHz): δ 8.38 (s, 1H), 8.14 (s, 1H), 8.11 (s, 1H), 7.82 (d, J=8.8 Hz, 1H), 7.77 (s, 1H), 7.54 (s, 1H), 7.38 (d, J=8.4 Hz, 1H), 7.20 (s, 1H), 6.89 (s, 1H), 4.13 (s, 3H), 3.65 (s, 3H), 3.34 (s, 3H), 3.11-3.09 (m, 1H), 2.32-2.25 (m, 2H), 1.81-1.72 (m, 5H), 1.25-1.23 (m, 1H), 1.10-1.14 (m, 1H), 1.02-0.93 (m, 2H), 0.67 (s, 3H).

### Example I-98: 9-(2,3-dihydrobenzo[b][1,4]dioxin-6-yl)-1-((1s,4s)-4-hydroxy-4-methylcyclohexyl)-3-methyl-8-(1-methyl-1H-pyrazol-4-yl)-1,3-dihydro-2H-imidazo[4,5-d]pyrrolo[1,2-b]pyridazin-2-one

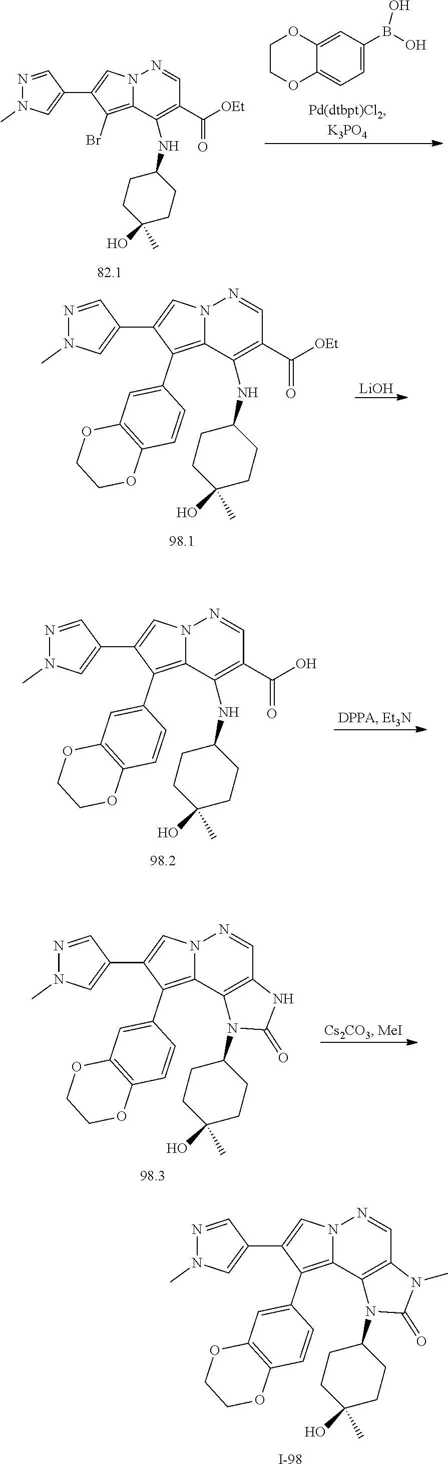

Synthesis of compound 98.1. Compound 98.1 was prepared from compound 82.1 and (2,3-dihydrobenzo[b][1,4]dioxin-6-yl)boronic acid following the procedure described in the synthesis of compound 59.12. The product was purified by flash column chromatography on silica gel (50% ethyl acetate in hexane). MS(ES): m/z 532.3 [M+H]⁺.

Synthesis of compound 98.2. Compound 98.2 was prepared from compound 98.1 following the procedure described in the synthesis of compound 1.10. The product was triturated with pentane, dried under vacuum and used in the next step without purification. MS(ES): m/z 504.2 [M+H]⁺.

Synthesis of compound 98.3. Compound 98.3 was prepared from compound 98.2 following the procedure described in the synthesis of compound 1.11. The product was purified by flash column chromatography on silica gel (Combiflash®, 1.8% methanol in DCM) MS(ES): m/z 501.4 [M+H]⁺.

Synthesis of compound I-98. Compound I-98 was prepared from compound 98.3 following the procedure described in the synthesis of compound 1.12. The product was purified by flash column chromatography on silica gel (1.4% methanol in DCM). MS(ES): m/z 515.3 [M+H]⁺, ¹H-NMR (400 MHz, DMSO-d₆): δ 8.34 (s, 1H), 8.08 (s, 1H), 7.17 (s, 1H), 7.01-6.97 (m, 2H), 6.82-6.80 (m, 2H), 4.39-4.42 (m, 4H), 3.91 (s, 1H), 3.71 (s, 3H), 3.51-3.45 (m, 1H), 3.33 (s, 3H), 2.63-2.54 (m, 2H), 1.40-1.32 (d, J=1.6 Hz, 2H), 1.10-1.06 (m, 2H), 0.99 (s, 3H), 0.67-0.12 (m, 2H).

### Example I-99: methyl ((1R,3R)-3-(8-(2,3-dihydropyrazolo[5,1-b]oxazol-7-yl)-9-(imidazo[1,5-a]pyridin-7-yl)-3-methyl-2-oxo-2,3-dihydro-1H-imidazo[4,5-d]pyrrolo[1,2-b]pyridazin-1-yl)cyclopentyl)carbamate

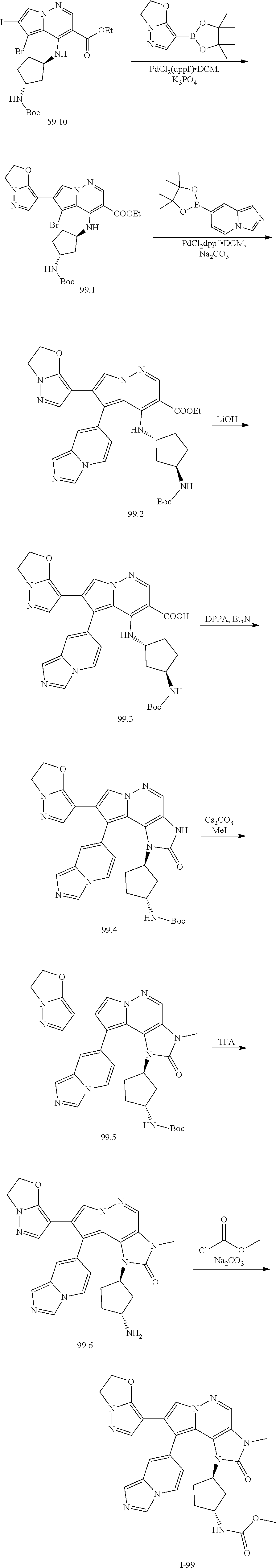

Synthesis of compound 99.1. Compound 99.1 was prepared from compound 59.10 and 7-(4,4,5,5-tetramethyl-1,3,2-dioxaborolan-2-yl)-2,3-dihydropyrazolo[5,1-b]oxazole following the procedure described in the synthesis of compound 54.3. The product was purified by flash column chromatography on silica gel (55% ethyl acetate in hexane). MS(ES): m/z 575.5 and 577.5 [M+H]⁺.

Synthesis of compound 99.2. Compound 99.2 was prepared from compound 99.1 and 7-(4,4,5,5-tetramethyl-1,3,2-dioxaborolan-2-yl)imidazo[1,5-a]pyridine following the procedure described in the synthesis of compound 1.13. The product was purified by flash column chromatography on silica gel (2.5% methanol in DCM). MS(ES): m/z 613.8 [M+H]⁺.

Synthesis of compound 99.3. Compound 99.3 was prepared from compound 99.2 following the procedure described in the synthesis of compound 1.10. The product was triturated with pentane, dried under vacuum and used in the next step without purification. MS(ES): m/z 585.7 [M+H]⁺.

Synthesis of compound 99.4. Compound 99.4 was prepared from compound 99.3 following the procedure described in the synthesis of compound 1.11. The product was purified by flash column chromatography on silica gel (Combiflash®, 2.0% methanol in DCM). MS(ES): m/z 582.5 [M+H]⁺.

Synthesis of compound 99.5. Compound 99.5 was prepared from compound 99.4 following the procedure described in the synthesis of compound 1.12. The product was purified by flash column chromatography on silica gel (1.8% methanol in DCM). MS(ES): m/z 596.8

Synthesis of compound 99.6. Compound 99.6 was prepared from compound 99.5 following the procedure described in the synthesis of compound 1.14. The product was triturated with n-pentane and dried under reduced pressure. It was used in the next step without purification. MS(ES): m/z 496.6 [M+H]⁺.

Synthesis of compound I-99. Compound I-99 was prepared from compound 99.6 following the procedure described in the synthesis of compound I-1. The product was purified by flash column chromatography on silica gel (Combiflash®, 3.2% methanol in DCM). MS (ES): m/z 554.27 [M+H]⁺, ¹H-NMR (400 MHz, DMSO-d₆): δ 8.41 (m, 3H), 7.82 (s, 1H), 7.56 (s, 1H), 7.36 (s, 1H), 6.84-6.82 (m, 2H), 6.66-6.64 (m, 1H), 5.09-5.05 (m, 2H), 4.23 (s, 3H), 3.99-3.93 (m, 1H), 3.49 (m, 2H), 3.44-3.41 (m, 1H), 3.36 (s, 3H), 2.16 (m, 1H), 1.84 (m, 1H), 1.58 (m, 1H), 1.33-1.29 (m, 1H), 1.05 (m, 1H), 0.08 (m, 1H).

### Example I-100: methyl ((1R,3R)-3-(8-(2,4-dimethyloxazol-5-yl)-3-methyl-9-(1-methyl-1H-indazol-5-yl)-2-oxo-2,3-dihydro-1H-imidazo[4,5-d]pyrrolo[1,2-b]pyridazin-1-yl)cyclopentyl)carbamate

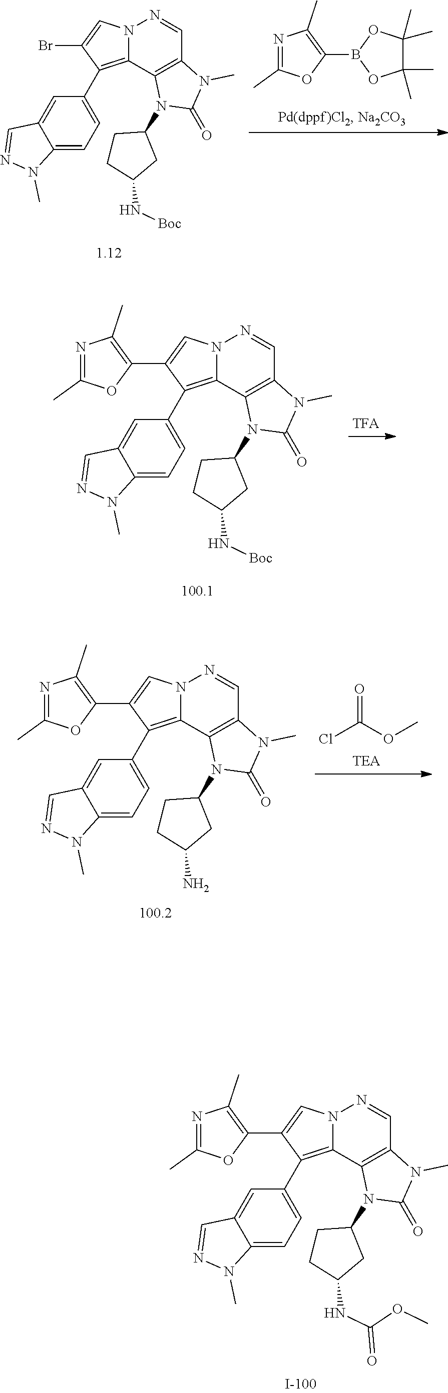

Synthesis of compound 100.1. Compound 100.1 was prepared from compound 1.12 and 2,4-dimethyl-5-(4,4,5,5-tetramethyl-1,3,2-dioxaborolan-2-yl)oxazole following the procedure described in the synthesis of compound 1.13. The product was purified by flash column chromatography on silica gel (3.0% methanol in DCM). MS(ES): m/z 597.4 [M+H]⁺.

Synthesis of compound 100.2. Compound 100.2 was prepared from compound 100.1 following the procedure described in the synthesis of compound 1.14. The product was used in the next step without purification. MS(ES): m/z 496.6 [M+H]⁺.

Synthesis of I-100. Compound I-100 was prepared from compound 100.2 following the procedure described in the synthesis of compound I-1. The product was purified by silica gel flash column chromatography on silica gel (3.2% methanol in DCM). MS(ES): m/z 555.28 [M+H]⁺, ¹H-NMR (400 MHz, DMSO-d₆): δ 8.50 (s, 1H), 8.12 (s, 1H), 8.02 (s, 1H), 7.71-7.66 (m, 2H), 7.36 (d, J=7.6 Hz, 1H), 6.66 (s, 1H), 4.10 (s, 3H), 3.87 (s, 1H), 3.44 (s, 3H), 3.44 (s, 1H), 3.34 (s, 3H), 2.11 (s, 3H), 1.99 (s, 1H), 1.84 (s, 3H), 1.80 (s, 1H), 1.51 (s, 1H), 1.11 (s, 1H), 0.81 (s, 1H), 0.36-−0.11 (m, 1H).

Example 1-101: (1R,2R)—N-((1s,4S)-4-(9-(benzo[d][1,3]dioxol-5-yl)-8-(2,3-dihydropyrazolo[5,1-b]oxazol-7-yl)-3-methyl-2-oxo-2,3-dihydro-1H-imidazo[4,5-d]pyrrolo[1,2-b]pyridazin-1-yl)-1-methylcyclohexyl)-2-fluorocyclopropane-1-carboxamide

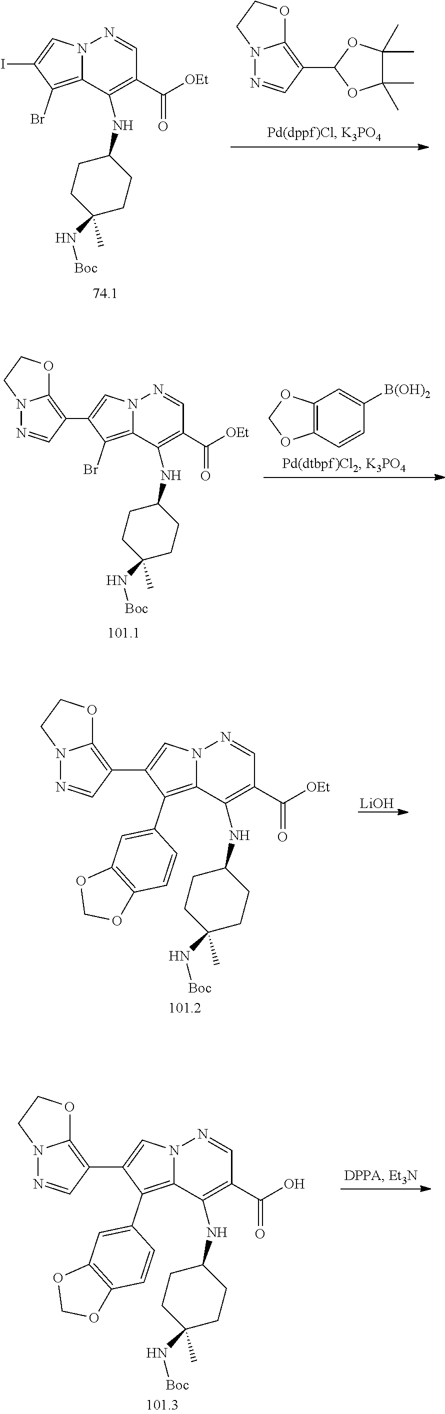

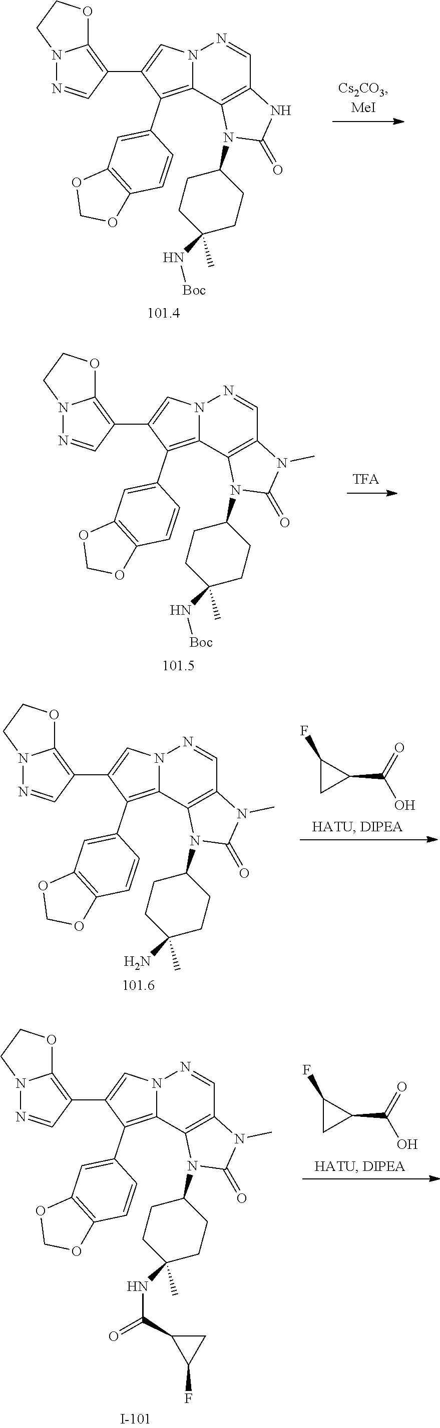 [SMILES: CC(C)(C)OC(=O)N[C@]1(C)CC[C@H](n2c(=O)[nH]c3cnn4cc(-c5cnn6c5OCC6)c(-c5ccc6c(c5)OCO6)c4c32)CC1 ; Cn1c(=O)n([C@H]2CC[C@@](C)(NC(=O)OC(C)(C)C)CC2)c2c1cnn1cc(-c3cnn4c3OCC4)c(-c3ccc4c(c3)OCO4)c21 ; Cn1c(=O)n([C@H]2CC[C@@](C)(N)CC2)c2c1cnn1cc(-c3cnn4c3OCC4)c(-c3ccc4c(c3)OCO4)c21 ; O=C(O)[C@H]1C[C@H]1F ; Cn1c(=O)n([C@H]2CC[C@@](C)(NC(=O)[C@H]3C[C@H]3F)CC2)c2c1cnn1cc(-c3cnn4c3OCC4)c(-c3ccc4c(c3)OCO4)c21 ; O=C(O)[C@H]1C[C@H]1F]

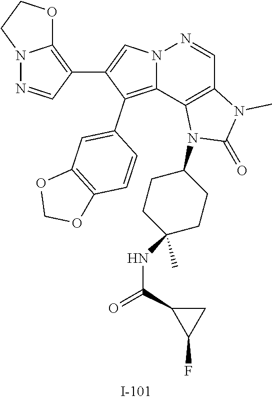 [SMILES: Cn1c(=O)n([C@H]2CC[C@@](C)(NC(=O)[C@H]3C[C@H]3F)CC2)c2c1cnn1cc(-c3cnn4c3OCC4)c(-c3ccc4c(c3)OCO4)c21]

Synthesis of compound 101.1. Compound 101.1 was prepared from compound 74.1.

Synthesis of compound 101.1. Compound 101.1 was prepared from compound 74.1 and 7-(4,4,5,5-tetramethyl-1,3,2-dioxaborolan-2-yl)-2,3-dihydropyrazolo[5,1-b]oxazole following the procedure described in the synthesis of compound 54.3. The product e was purified by flash column chromatography on silica gel (55% ethyl acetate in hexane). MS(ES): m/z 603.4 and 605.4 [M+H]⁺.

Synthesis of compound 101.2. Compound 101.2 was prepared from compound 101.1 and benzo[d][1,3]dioxol-5-ylboronic acid following the procedure described in the synthesis of compound 59.12. The product was purified by flash column chromatography on silica gel (55% ethyl acetate in hexane). MS(ES): m/z 645.6 [M+H]⁺.

Synthesis of compound 101.3 Compound 101.3 was prepared from compound 101.2 following the procedure described in the synthesis of compound 1.10. The product was triturated with pentane, dried under vacuum and used in the next step without purification. MS(ES): m/z 617.6 [M+H]⁺.

Synthesis of compound 101.4. Compound 101.4 was prepared from compound 101.3 following the procedure described in the synthesis of compound 1.11. The product was purified by flash column chromatography on silica gel (Combiflash®, 2.1% methanol in DCM). MS(ES): m/z 614.6 [M+H]⁺.

Synthesis of compound 101.5. Compound 101.5 was prepared from compound 101.4 following the procedure described in the synthesis of compound 1.12. The product was purified by flash column chromatography on silica gel (1.8% methanol in DCM). MS(ES): m/z 628.7 [M+H]⁺.

Synthesis of compound 101.6. Compound 101.6 was prepared from compound 101.5 following the procedure described in the synthesis of compound 1.14. The product was used in the next step without purification. MS(ES): m/z 528.4 [M+H]⁺.

Synthesis of compound I-101. Compound I-101 was prepared from compound 101.6 and (1R,2R)-2-fluorocyclopropane-1-carboxylic acid following the procedure described in the synthesis of compound I-45. The product was purified by flash column chromatography on silica gel (Combiflash®, 3.2% methanol in DCM). MS (ES): m/z 614.65 [M+H]⁺, ¹H-NMR (400 MHz, DMSO-d₆): δ 8.34 (s, 1H), 7.76 (s, 1H), 7.45 (s, 1H), 7.07 (d, J=7.6 Hz, 1H), 6.95 (s, 1H), 6.85 (d, J=8.0 Hz, 1H), 6.36 (s, 1H), 6.12 (d, J=10.4 Hz, 2H), 5.13 (t, J=8.0 Hz, 2H), 4.24 (t, J=8.0 Hz, 2H), 3.49 (s, 1H), 3.40 (s, 1H), 2.20-2.00 (m, 2H), 1.86-1.82 (m, 1H), 1.46-1.41 (m, 2H), 1.33-1.16 (m, 3H), 1.11 (s, 5H), 0.95-0.91 (m, 2H), 0.55-0.44 (m, 2H).

### Example I-102: 9-(benzo[d][1,3]dioxol-5-yl)-8-(5,6-dihydro-4H-pyrrolo[1,2-b]pyrazol-3-yl)-1-((1s,4s)-4-hydroxy-4-methylcyclohexyl)-3-methyl-1,3-dihydro-2H-imidazo[4,5-d]pyrrolo[1,2-b]pyridazin-2-one

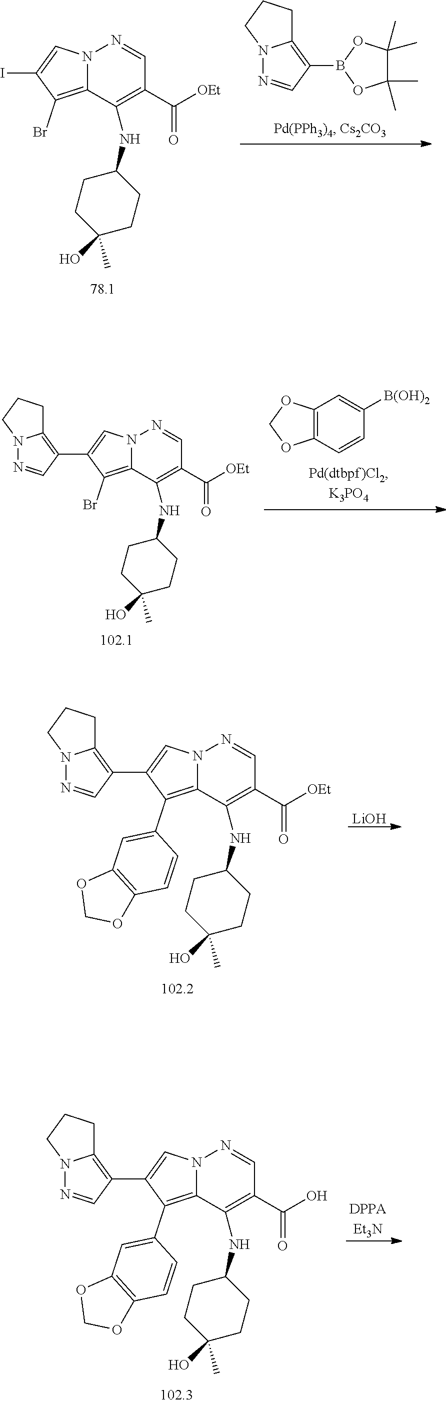

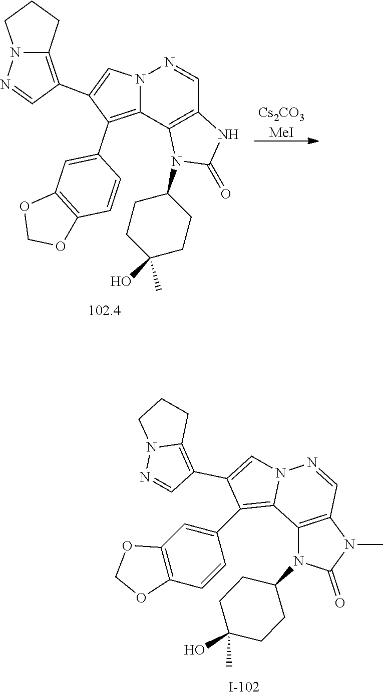

Synthesis of compound 102.1. A mixture of 78.1 (0.960 g, 1.84 mmol, 1.0 equiv), 3-(4,4,5,5-tetramethyl-1,3,2-dioxaborolan-2-yl)-5,6-dihydro-4H-pyrrolo[1,2-b]pyrazole (0.861 g, 3.68 mmol, 2.0 equiv) and cesium carbonate (1.79 g, 5.51 mmol, 3.0 equiv) in 1,4-dioxane (12 mL) and water (3 mL) was degassed by bubbling through a stream of argon for 10 min. Tetrakis(triphenylphosphine) palladium (0) (0.106 g, 0.091 mmol, 0.05 equiv) was added and degassed for 5 min. The reaction mixture was stirred at 80° C. for 2 h. It was cooled to room temperature. The reaction mixture was extracted with ethyl acetate. The combined organic layers were washed with brine, dried over anhydrous sodium sulfate, filtered and concentrated under reduced pressure. The residue was purified by flash column chromatography on silica gel (Combiflash®, 2.4% methanol in DCM) to afford (0.39 g, 42%). MS(ES): m/z 502.4 and 504.4 [M+H]⁺.

Synthesis of compound 102.2. Compound 102.2 was prepared from compound 102.1 and benzo[d][1,3]dioxol-5-ylboronic acid following the procedure described in the synthesis of compound 59.12. The product was purified by flash column chromatography on silica gel (2.5% methanol in DCM). MS(ES): m/z 544.51 [M+H]⁺.

Synthesis of compound 102.3. Compound 102.3 was prepared from compound 102.2 following the procedure described in the synthesis of compound 1.10. The product was triturated with pentane, dried under vacuum and used in the next step without purification. MS(ES): m/z 516.18 [M+H]⁺.

Synthesis of compound 102.4. Compound 102.4 was prepared from compound 102.3 following the procedure described in the synthesis of compound 1.11. The product was purified by flash column chromatography on silica gel (Combiflash®, 3.2% methanol in DCM). MS(ES): m/z 513.10 [M+H]⁺.

Synthesis of compound I-102. Compound I-102 was prepared from compound 102.4 following the procedure described in the synthesis of compound 1.12. The product was purified by flash column chromatography on silica gel (3.2% methanol in DCM). MS(ES): m/z 527.12 [M+H]⁺, ¹H-NMR (400 MHz, DMSO-d₆): δ 8.36 (s, 1H), 7.91 (s, 1H), 7.04 (d, J=8.0 Hz, 1H), 6.91 (s, 1H), 6.82 (d, J=8.0 Hz, 1H), 6.60 (s, 1H), 6.13 (s, 1H), 6.08 (s, 1H), 4.02-3.98 (m, 2H), 3.90 (s, 1H), 3.46 (m, 1H), 3.34 (s, 3H), 2.78-2.75 (m, 2H), 1.42-1.38 (m, 2H), 1.35-0.33 (m, 1H), 1.30 (m, 2H), 1.13-1.10 (m, 1H), 1.06-1.03 (m, 1H), 0.98 (s, 3H), 0.87-0.83 (m, 1H), 0.66-0.63 (m, 2H).

### Example I-103: methyl ((1R,3R)-3-(3-methyl-8-(1-methyl-1H-pyrazol-4-yl)-2-oxo-9-(1,2,3,4-tetrahydroquinolin-7-yl)-2,3-dihydro-1H-imidazo[4,5-d]pyrrolo[1,2-b]pyridazin-1-yl)cyclopentyl)carbamate

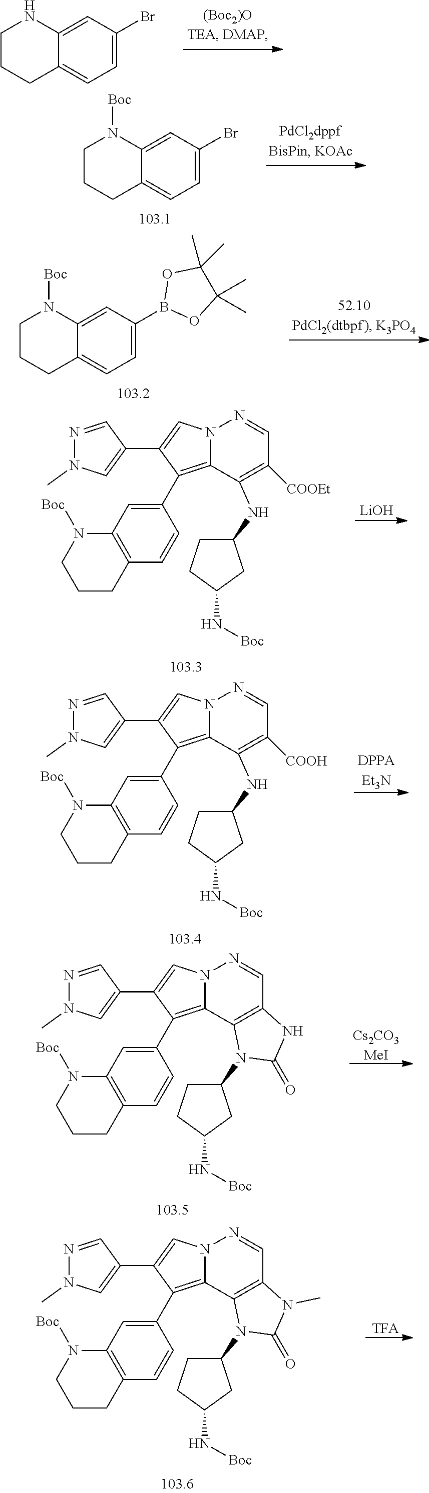

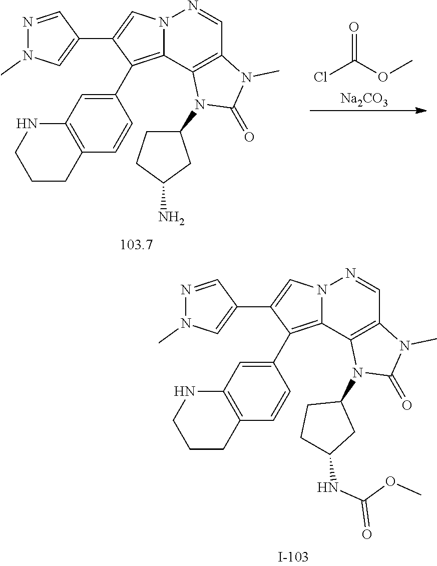

Synthesis of compound 103.1. To a solution of 7-bromo-1,2,3,4-tetrahydroquinoline (1 g, 4.73 mmol, 1.0 equiv), dimethyl aminopyridine (0.578 g, 4.73 mmol, 1.0 equiv) and triethylamine (0.957 g, 9.47 mmol, 2.0 equiv) in THF (8.0 mL) was added (Boc)₂O (2.06 g, 9.47 mmol, 2.0 equiv) and stirred at room temperature for 2 h. It was transferred into water and extracted with ethyl acetate. The combined organic layers were washed with brine, dried over anhydrous sodium sulfate, filtered and concentrated under reduced pressure. The residue was purified by flash column chromatography on silica gel (55% ethyl acetate in hexane) to afford 103.1 (0.510 g, 35%). MS(ES): m/z 312.4 and 314.4 [M+H]⁺.

Synthesis of compound 103.2. A mixture of 103.1 (0.510 g, 1.6 mmol, 1.0 equiv), bis(pinacolato)diboron (0.619 g, 2.45 mmol, 1.5 equiv) and potassium acetate (0.48 g, 4.9 mmol, 3 equiv) in 1,4-dioxane (15.0 mL) was degassed by bubbling through a stream of argon for 10 min. 1,1′-Bis(diphenylphosphino)ferrocene) palladium (II) dichloride (0.119 g, 0.163 mmol, 0.1 equiv) was added and degassed for 5 min. The reaction mixture was stirred at 100° C. for 2 h. This reaction was transferred into water and extracted with ethyl acetate. The combined organic layers were washed with brine, dried over anhydrous sodium sulfate, filtered and concentrated under reduced pressure. The residue was purified by flash column chromatography on silica gel (42% ethyl acetate in hexane) to afford 103.2 (0.41 g, 70%). MS(ES): m/z 360.2 [M+H]⁺.

Synthesis of compound 103.3. Compound 103.3 was prepared from compound 103.2 and 52.10 following the procedure described in the synthesis of compound 59.12. The product was purified by flash column chromatography on silica gel (58% ethyl acetate in hexane). MS(ES): m/z 700.3 [M+H]⁺.

Synthesis of compound 103.4. Compound 103.4 was prepared from compound 103.3 following the procedure described in the synthesis of compound 1.10. The product was triturated with pentane, dried under vacuum and used in the next step without purification. MS(ES): m/z 672.3 [M+H]⁺.

Synthesis of compound 103.5. Compound 103.5 was prepared from compound 103.4 following the procedure described in the synthesis of compound 1.11. The product was purified by flash column chromatography on silica gel (Combiflash®, 2.1% methanol in DCM). MS(ES): m/z 669.4 [M+H]⁺.

Synthesis of compound 103.6. Compound 103.6 was prepared from compound 103.5 following the procedure described in the synthesis of compound 1.12. The product was purified by flash column chromatography on silica gel (1.8% methanol in DCM). MS(ES): m/z 683.5 [M+H]⁺.

Synthesis of compound 103.7. Compound 103.7 was prepared from compound 103.6 following the procedure described in the synthesis of compound 1.14. The product was used in the next step without purification. MS(ES): m/z 483.2 [M+H]⁺.

Synthesis of compound I-103. Compound I-103 was prepared from compound 103.7 following the procedure described in the synthesis of compound I-1. The product was purified by flash column chromatography on silica gel (Combiflash®, 3.2% methanol in DCM). MS (ES): m/z 541.3 [M+H]⁺, ¹H-NMR (400 MHz, DMSO-d₆): δ 8.32 (s, 1H), 8.06 (s, 1H), 7.43 (s, 1H), 7.20-7.16 (m, 1H), 6.98-6.93 (m, 1H), 6.86-6.71 (m, 1H), 6.45-6.35 (m, 2H), 4.03 (bs, 2H), 3.88 (bs, 1H), 3.82 (s, 3H), 3.50 (s, 3H), 3.19 (s, 2H), 2.81-2.73 (m, 2H), 2.17-2.02 (m, 2H), 1.85 (s, 4H), 1.60-152 (m, 1H), 1.44-1.38 (m, 1H), 1.10-1.06 (m, 1H), 0.89-0.82 (m, 1H).

### Example I-104: methyl ((1R,3R)-3-(8-(3,6-dihydro-2H-pyran-4-yl)-3-methyl-2-oxo-9-(6-oxo-1,6-dihydropyridazin-3-yl)-2,3-dihydro-1H-imidazo[4,5-d]pyrrolo[1,2-b]pyridazin-1-yl)cyclopentyl)carbamate

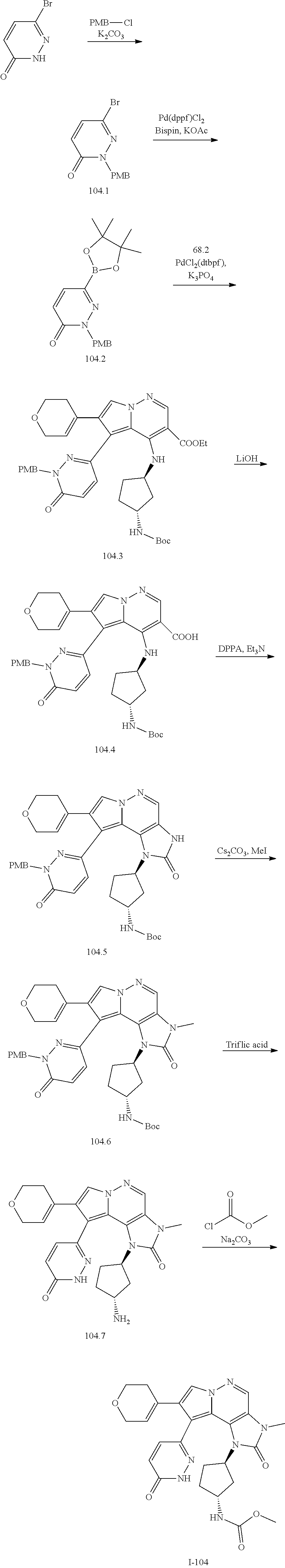

Synthesis of compound 104.1. To a stirred solution of 6-bromopyridazin-3(2H)-one (3.0 g, 17.14 mmol, 1.0 equiv) in DMF (40 mL) was added potassium carbonate (7.13 g, 51.72 mmol, 3.0 equiv) and 4-methoxybenzyl chloride (4.03 g, 25.86 mmol, 1.5 equiv) at 0° C. The reaction mixture was stirred at room temperature for 1 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (20% ethyl acetate in hexane) to afford 104.1 (1.7 g, 34%). MS (ES): m/z 295.14 and 297.14 [M+H]⁺.

Synthesis of compound 104.2. A mixture of 104.1 (1.7 g, 5.76 mmol, 1.0 equiv), bis(pinacolato)diboron (4.37 g, 17.28 mmol, 3.0 equiv) and potassium acetate (1.69 g, 17.28 mmol, 3.0 equiv) in dioxane (30 mL) was degassed by bubbling through a stream of argon for 10 min. [1,1′-Bis(diphenylphosphino)ferrocene]dichloropalladium(II) (0.421 mg, 0.57 mmol, 0.1 equiv) was added and degassed for 5 min. The reaction mixture was stirred at 80° C. for 1 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (25% ethyl acetate in hexane) to afford 104.2 (0.79 g, 40%). MS (ES): m/z 343.20 [M+H]⁺.

Synthesis of compound 104.3. Compound 104.3 was prepared from compound 104.2 and 68.2 following the procedure described in the synthesis of compound 59.12. The product was purified by flash column chromatography on silica gel (2.8% methanol in DCM). MS (ES): m/z 685.79 [M+H]⁺.

Synthesis of compound 104.4. Compound 104.4 was prepared from compound 104.3 following the procedure described in the synthesis of compound 1.10. The product was triturated with n-pentane, dried under vacuum and used in the next step without purification. MS (ES): m/z 657.6 [M+H]⁺.

Synthesis of compound 104.5. Compound 104.5 was prepared from compound 104.4 following the procedure described in the synthesis of compound 1.11. The product was purified by flash column chromatography on silica gel (Combiflash®, 2.5% methanol in DCM). MS (ES): m/z 654.5 [M+H]⁺.

Synthesis of compound 104.6. Compound 104.6 was prepared from compound 104.5 following the procedure described in the synthesis of compound 1.12. The product was purified by flash column chromatography on silica gel (2.5% methanol in DCM). MS (ES): m/z 668.3 [M+H]⁺.

Synthesis of compound 104.7. To a solution of 104.6 (0.089 g, 0.133 mmol, 1.0 equiv) in DCM (4 mL) was added trifluoromethanesulfonic acid (1 mL) at 0° C. dropwise. The reaction mixture was stirred at room temperature for 10 min. It was transferred into ice-water, neutralized with sodium bicarbonate and extracted with DCM. The combined organic layers were washed with brine, dried over anhydrous sodium sulfate, filtered and concentrated under reduced pressure. The residue was triturated with n-pentane and dried under reduced pressure. It was used in the next step without purification. MS (ES): m/z 448.3 [M+H]⁺.

Synthesis of compound I-104. Compound I-104 was prepared from compound 104.7 following the procedure described in the synthesis of compound I-1. The product was purified by flash column chromatography on silica gel (Combiflash®, 3.5% methanol in DCM). MS (ES): m/z 506.2 [M+H]⁺. ¹H NMR (400 MHz, DMSO-d₆): δ 13.27 (s, 1H), 8.51 (s, 1H), 8.02 (s, 1H), 7.50 (d, J=9.6 Hz, 1H), 7.08 (d, J=6.8 Hz, 1H), 6.99 (dd, J=2.4, 9.6 Hz, 1H), 5.52 (s, 1H), 4.16-4.17 (m, 1H), 4.07-4.07 (m, 3H), 3.74-3.77 (m, 2H), 3.52 (s, 3H), 3.40 (s, 3H), 2.32-2.35 (m, 2H), 2.21-2.23 (m, 1H), 2.10 (m, 1H), 1.74-1.77 (m, 1H), 1.47 (m, 1H), 1.06-1.10 (m, 1H), 1.25 (m, 1H).

### Example I-105: methyl ((1R,3R)-3-(8-(3-methoxy-1-methyl-1H-pyrazol-4-yl)-3-methyl-9-(5-methyl-6-oxo-1,6-dihydropyridazin-3-yl)-2-oxo-2,3-dihydro-1H-imidazo[4,5-d]pyrrolo[1,2-b]pyridazin-1-yl)cyclopentyl)carbamate

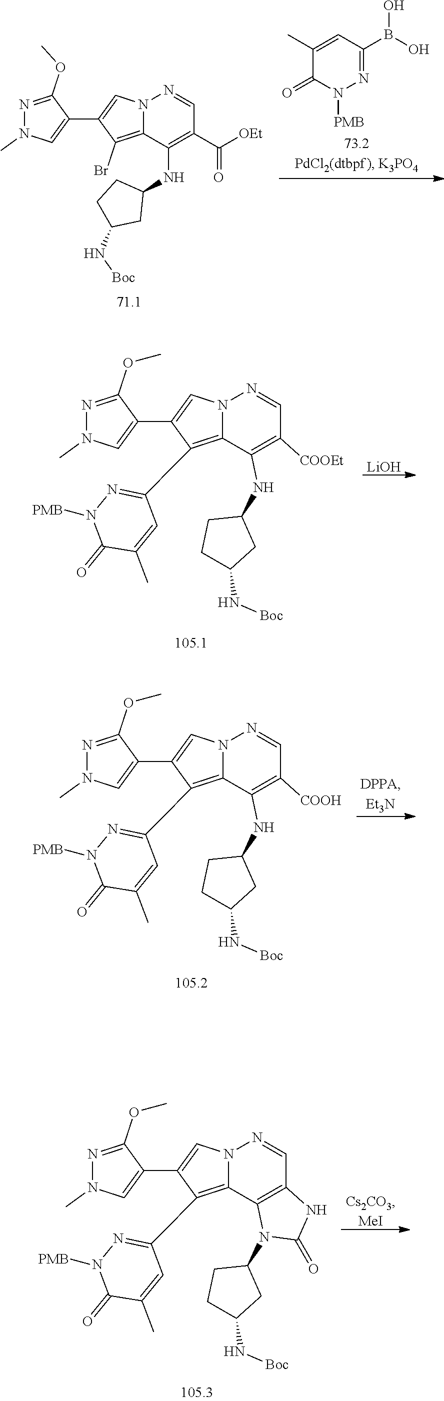

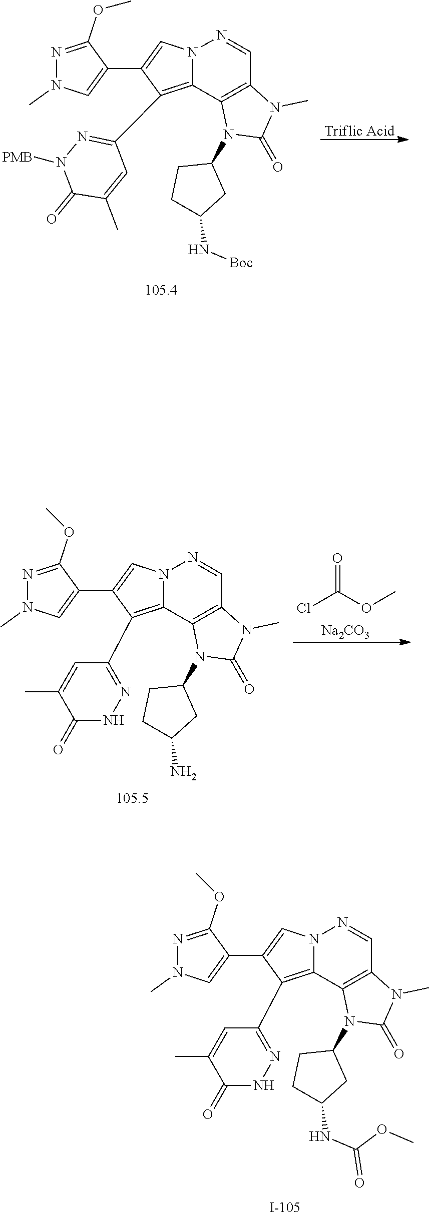

Synthesis of compound 105.1. Compound 105.1 was prepared from compound 71.1 and 73.2 following the procedure described in the synthesis of compound 59.12. The product was purified by flash column chromatography on silica gel (Combiflash®, 0-3.2% methanol in DCM). MS(ES): m/z 727.3 [M+H]⁺.

Synthesis of compound 105.2. Compound 105.2 was prepared from compound 105.1 following the procedure described in the synthesis of compound 1.10. The product was triturated with pentane, dried under vacuum and used in the next step without purification. MS(ES): m/z 699.20 [M+H]⁺.

Synthesis of compound 105.3. Compound 105.3 was prepared from compound 105.2 following the procedure described in the synthesis of compound 1.11. The product was purified by flash column chromatography on silica gel (Combiflash®, 1.8% methanol in DCM). MS(ES): m/z 696.31 [M+H]⁺.

Synthesis of compound 105.4. Compound 105.4 was prepared from compound 105.3 following the procedure described in the synthesis of compound 1.12. The product was purified by flash column chromatography on silica gel (1.4% methanol in DCM). MS(ES): m/z 710.20 [M+H]⁺.

Synthesis of compound 105.5. Compound 105.5 was prepared from compound 105.4 following the procedure described in the synthesis of compound 21.8. The product was triturated with pentane and dried under reduced pressure. It was used in the next step without purification. MS(ES): m/z 490.2 [M+H]⁺.

Synthesis of compound I-105. Compound I-105 was prepared from compound 105.5 following the procedure described in the synthesis of compound I-1. The product was purified by flash column chromatography on silica gel (Combiflash®, 2.6% methanol in DCM). MS(ES): m/z 548.3 [M+H]⁺, #H-NMR (400 MHz, DMSO-d₆): δ13.07 (s, 1H), 8.46 (s, 1H), 7.99 (s, 1H), 7.36 (m, 1H), 7.05 (m, 2H), 4.15 (m, 1H), 4.03 (m, 1H), 3.83 (s, 3H), 3.64 (s, 3H), 3.50 (s, 3H), 3.33 (s, 3H), 2.12 (s, 3H), 2.03-1.99 (m, 3H), 1.70 (m, 1H), 1.42 (m, 1H), 1.01 (m, 1H).

### Example I-106: (1R,2R)—N-((1s,4S)-4-(9-(2,3-dihydrobenzo[b][1,4]dioxin-6-yl)-3-methyl-8-(1-methyl-1H-pyrazol-4-yl)-2-oxo-2,3-dihydro-1H-imidazo[4,5-d]pyrrolo[1,2-b]pyridazin-1-yl)-1-methylcyclohexyl)-2-fluorocylopropane-1-carboxamide

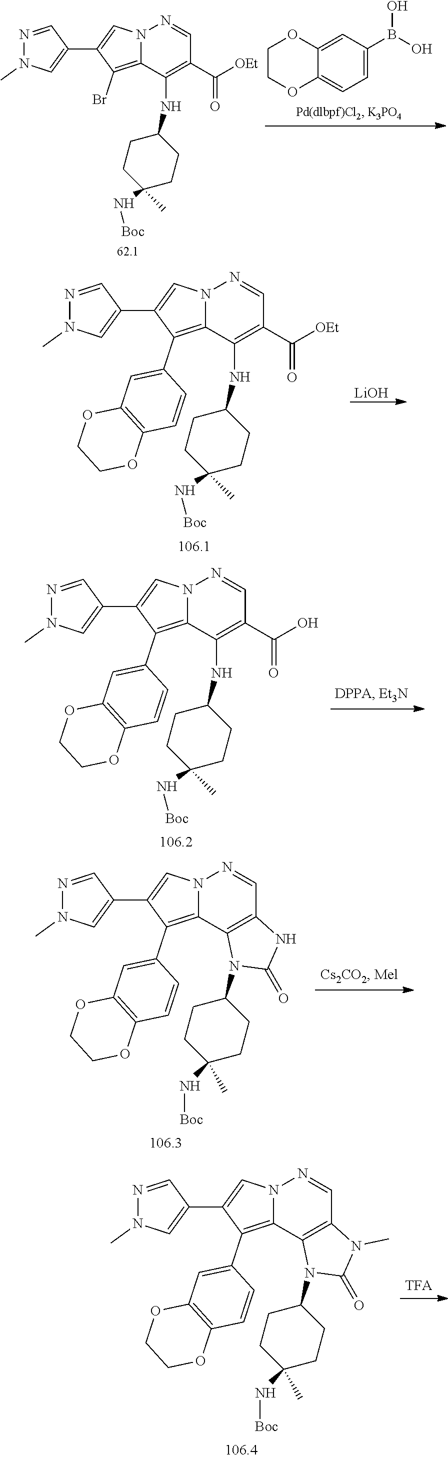

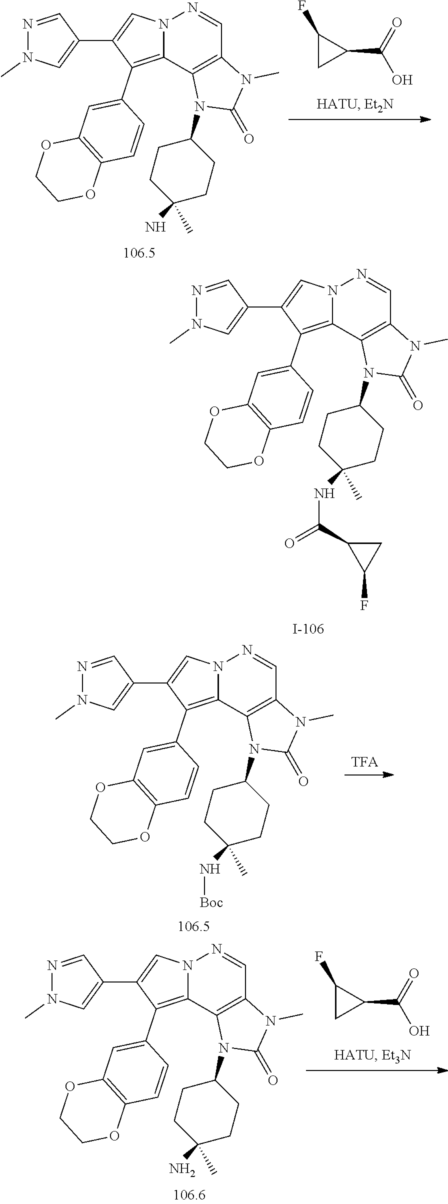

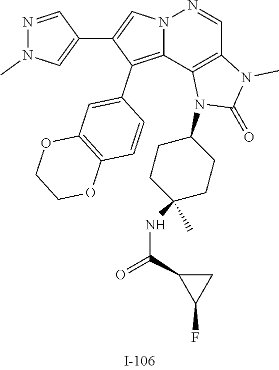

Synthesis of compound 106.1. Compound 106.1 was prepared from compound 62.1.

Synthesis of compound 106.1. Compound 106.1 was prepared from compound 62.1 and (2,3-dihydrobenzo[b][1,4]dioxin-6-yl)boronic acid following the procedure described in the synthesis of compound 59.12. The product was purified by flash column chromatography on silica gel (48% ethyl acetate in n-hexane). MS (ES): m/z 631.5 [M+H]⁺.

Synthesis of compound 106.2. Compound 106.2 was prepared from compound 106.1 following the procedure described in the synthesis of compound 1.10. The product was used in the next step without purification. MS (ES): m/z 603.6 [M+H]⁺.

Synthesis of compound 106.3. Compound 106.3 was prepared from compound 106.2 following the procedure described in the synthesis of compound 1.11. The product was purified by flash column chromatography on silica gel (2.5% methanol in DCM). MS (ES): m/z 600.6 [M+H]⁺.

Synthesis of compound 106.4. Compound 106.4 was prepared from compound 106.3 following the procedure described in the synthesis of compound 1.12. The product was purified by flash column chromatography on silica gel (2.6% methanol in DCM). MS (ES): m/z 614.7 [M+H]⁺.

Synthesis of compound 106.5. Compound 106.5 was prepared from compound 106.4 following the procedure described in the synthesis of compound 1.14. MS (ES): m/z 514.6 [M+H]⁺. The product was used in the next step without purification.

Synthesis of compound I-106. Compound I-106 was prepared from compound 106.5 and (1R,2R)-2-fluorocyclopropane-1-carboxylic acid following the procedure described in the synthesis of compound I-45. The product was purified by flash column chromatography on silica gel (3.5% methanol in DCM). MS (ES): m/z 600.5 [M+H]⁺, ¹H NMR (DMSO-d₆, 400 MHz): δ 8.35 (s, 1H), 8.09 (s, 1H), 7.46 (s, 1H), 7.20 (d, J=4.4 Hz, 1H), 7.02 (t, J=8.4 Hz, 2H), 6.84 (t, J=7.2 Hz, 2H), 4.73 (dd, J=2.80, 66.8 Hz, 1H), 4.36 (m, 3H), 4.28-4.24 (m, 2H), 3.72 (m, 4H), 3.48 (m, 1H), 3.34 (s, 4H), 2.15-1.99 (m, 3H), 1.84 (m, 1H), 1.46-1.40 (m, 2H), 0.96-0.91 (m, 2H), 0.56-0.50 (m, 3H).

### Example I-107: 1-((1s,4s)-4-hydroxy-4-methylcyclohexyl)-8-(3-methoxy-1-methyl-1H-pyrazol-4-yl)-3-methyl-9-(1-methyl-1H-indazol-5-yl)-1,3-dihydro-2H-imidazo[4,5-d]pyrrolo[1,2-b]pyridazin-2-one

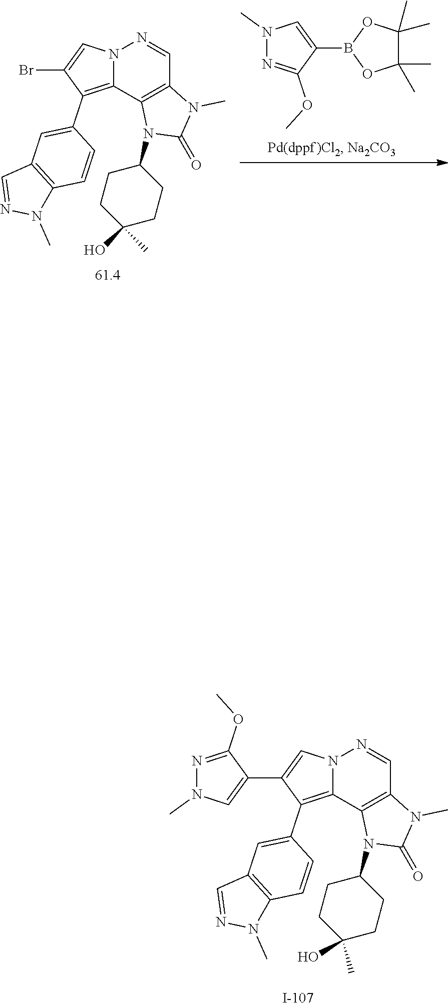

Synthesis of compound I-107. Compound I-107 was prepared from compound 61.4 and 3-methoxy-1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (3% methanol in DCM). MS(ES): m/z 541.4 [M+H]⁺, ¹H-NMR (400 MHz, DMSO-d₆): δ 8.37 (s, 1H), 8.11 (d, J=0.8 Hz, 1H), 8.06 (s, 1H), 7.81 (s, 1H), 7.74 (d, J=0.4 Hz, 1H), 7.36 (dd, J=1.6, 8.8 Hz, 1H), 6.08 (s, 1H), 4.12 (s, 3H), 3.87 (s, 3H), 3.55 (s, 1H), 3.38 (m, 3H), 3.36 (m, 3H), 3.32 (m, 1H), 2.46-2.46 (m, 2H), 1.16-1.13 (m, 1H), 1.12-1.10 (m, 3H), 0.57 (s, 3H), 0.09-0.08 (m, 1H), −0.46-−0.47 (m, 1H).

### Example I-108: (1R,2R)—N-((1s,4S)-4-(8-(3,6-dihydro-2H-pyran-4-yl)-3-methyl-9-(1-methyl-1H-indazol-5-yl)-2-oxo-2,3-dihydro-1H-imidazo[4,5-d]pyrrolo[1,2-b]pyridazin-1-yl)-1-methylcyclohexyl)-2-fluorocyclopropane-1-carboxamide

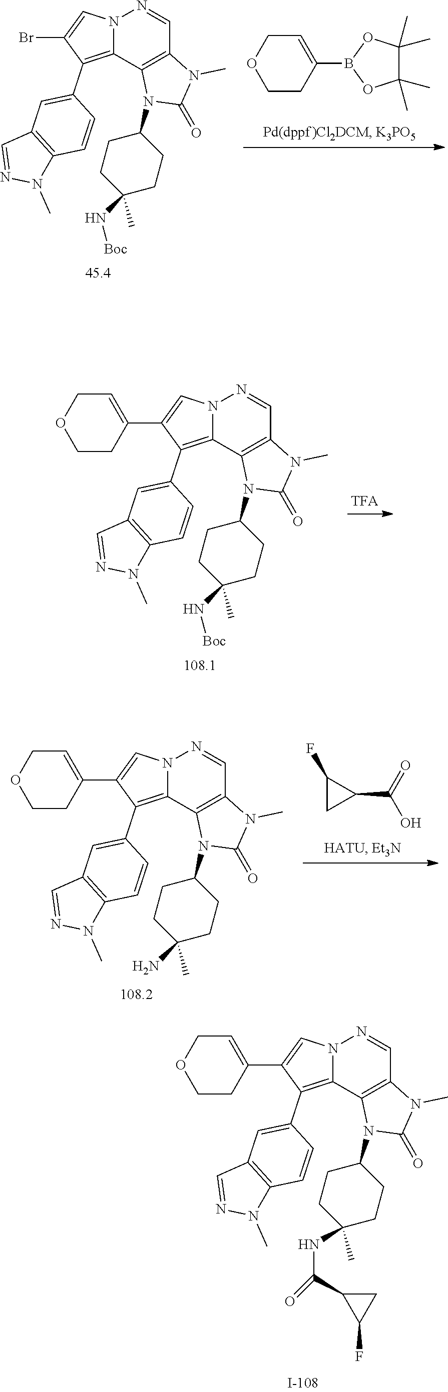

Synthesis of compound 108.1. Compound 108.1 was prepared from compound 45.4 and 2-(3,6-dihydro-2H-pyran-4-yl)-4,4,5,5-tetramethyl-1,3,2-dioxaborolane following the procedure described in the synthesis of compound 54.3. The product was purified by flash column chromatography on silica gel (Combiflash®, 0-2.8% methanol in DCM). MS(ES): m/z 612.4 [M+H]⁺.

Synthesis of compound 108.2. Compound 108.2 was prepared from compound 108.1 following the procedure described in the synthesis of compound 1.14. The product was triturated with pentane and dried under reduced pressure. It was used in the next step without purification. MS(ES): MS m/z: 512.4 [M+H]⁺.

Synthesis of I-108. Compound I-108 was prepared from compound 108.2 and (1R,2R)-2-fluorocyclopropane-1-carboxylic acid following the procedure described in the synthesis of compound I-45. The product was purified by flash column chromatography on silica gel (Combiflash®, 0-2.4% methanol in DCM). MS(ES): MS m/z: 598.4 [M+H]⁺, ¹H NMR (DMSO-d₆, 400 MHz): δ 8.41 (s, 1H), 8.11 (s, 1H), 7.94 (s, 1H), 7.80 (d, J=8.4 Hz, 1H), 7.75 (s, 1H), 7.39 (d, J=8.8 Hz, 1H), 7.27 (s, 1H), 5.30 (s, 1H), 4.80-4.60 (m, 1H), 4.12 (s, 3H), 3.88 (s, 2H), 3.61 (t, J=5.6 Hz, 2H), 2.98 (bs, 1H), 2.34-2.25 (m, 2H), 2.12 (s, 2H), 1.78-1.74 (m, 2H), 1.70-1.50 (m, 1H), 1.41-1.34 (m, 1H), 1.27-1.23 (m, 2H), 1.09-0.86 (m, 3H), 0.69 (s, 3H) −0.01 (bs, 1H).

### Example I-109: methyl ((1R,3R)-3-(9-(5-cyclopropyl-6-oxo-1,6-dihydropyridazin-3-yl)-8-(3,6-dihydro-2H-pyran-4-yl)-3-methyl-2-oxo-2,3-dihydro-1H-imidazo[4,5-d]pyrrolo[1,2-b]pyridazin-1-yl)cyclopentyl)carbamate

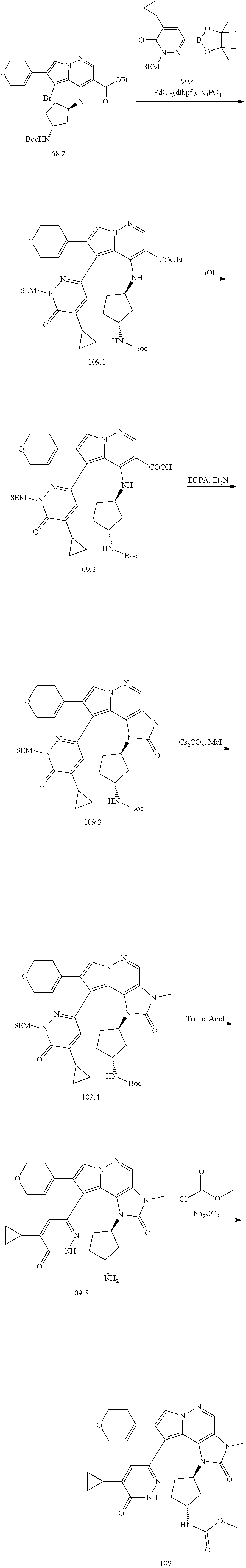

Synthesis of compound 109.1. Compound 109.1 was prepared from compound 68.2 and 90.4 following the procedure described in the synthesis of compound 59.12. The product was purified by flash column chromatography on silica gel (2.1% methanol in DCM). MS(ES): m/z 735.85 [M+H]⁺.

Synthesis of compound 109.2. Compound 109.2 was prepared from compound 109.1 following the procedure described in the synthesis of compound 1.10. The product was triturated with pentane, dried under vacuum and used in the next step without purification. MS(ES): m/z 707.51 [M+H]⁺.

Synthesis of compound 109.3. Compound 109.3 was prepared from compound 109.2 following the procedure described in the synthesis of compound 1.11. The product was purified by flash column chromatography on silica gel (Combiflash®, 2.6% methanol in DCM). MS(ES): m/z 704.80 [M+H]⁺.

Synthesis of compound 109.4. Compound 109.4 was prepared from compound 109.3 following the procedure described in the synthesis of compound 1.12. The product was purified by flash column chromatography on silica gel (3.1% methanol in DCM). MS(ES): m/z 718.50 [M+H]⁺.

Synthesis of compound 109.5. Compound 109.5 was prepared from compound 109.4 following the procedure described in the synthesis of compound 21.8. MS(ES): m/z 488.20 [M+H]⁺. The product was used in next step without purification.

Synthesis of compound I-109. Compound I-109 was prepared from compound 109.5 following the procedure described in the synthesis of compound I-1. The product was purified by flash column chromatography on silica gel (Combiflash®, 3.4% methanol in DCM). MS (ES): m/z 546.19 [M+H]⁺, ¹H-NMR (400 MHz, DMSO-d₆): δ 13.96 (s, 1H), 8.53 (s, 1H), 8.06 (s, 1H), 6.98 (s, 1H), 6.91 (s, 1H), 5.39 (m, 1H), 4.11 (m, 1H), 4.01 (m, 2H), 3.72-3.70 (m, 2H), 3.49 (s, 3H), 3.39 (s, 3H), 2.23 (m, 2H), 2.11 (m, 2H), 1.98-1.93 (m, 3H), 1.55-1.52 (m, 1H), 1.34-1.32 (m, 2H), 0.95-0.90 (m, 4H).

### Example I-110: 8-(3-fluoro-1-methyl-1H-pyrazol-4-yl)-1-((1s,4s)-4-hydroxy-4-methylcyclohexyl)-3-methyl-9-(1-methyl-1H-indazol-5-yl)-1,3-dihydro-2H-imidazo[4,5-d]pyrrolo[1,2-b]pyridazin-2-one

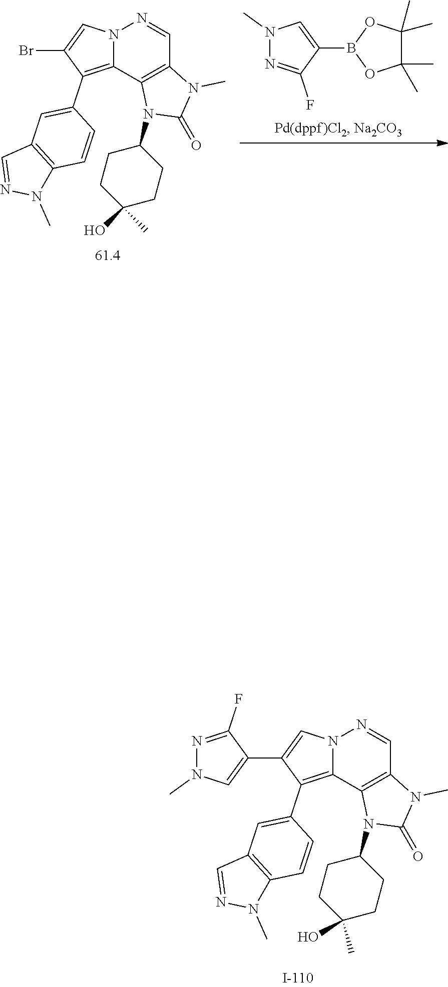

Synthesis of compound I-110. Compound I-110 was prepared from compound 61.4 and 3-fluoro-1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (3% methanol in DCM). MS(ES): m/z 529.5 [M+H]⁺, ¹H-NMR (400 MHz, DMSO-d₆): δ 8.43 (s, 1H), 8.10 (s, 1H), 7.98 (d, J=1.2 Hz, 1H), 7.78 (d, J=8.4 Hz, 1H), 7.75 (s, 1H), 7.36 (dd, J=1.2 Hz, 8.4 Hz, 1H), 6.56 (d, J=2.0 Hz, 1H), 4.11 (s, 3H), 3.50 (s, 3H), 3.37-3.35 (m, 4H), 3.08-3.02 (m, 1H), 2.46-2.39 (m, 2H), 1.16-1.13 (m, 1H), 0.95-0.87 (m, 3H), 0.56 (s, 3H), 0.10-0.04 (m, 1H), −0.44-−0.49 (m, 1H).

### Example I-111: 1-((1s,4s)-4-hydroxy-4-methylcyclohexyl)-9-(4-methoxyphenyl)-3-methyl-8-(1-methyl-1H-pyrazol-4-yl)-1,3-dihydro-2H-imidazo[4,5-d]pyrrolo[1,2-b]pyridazin-2-one

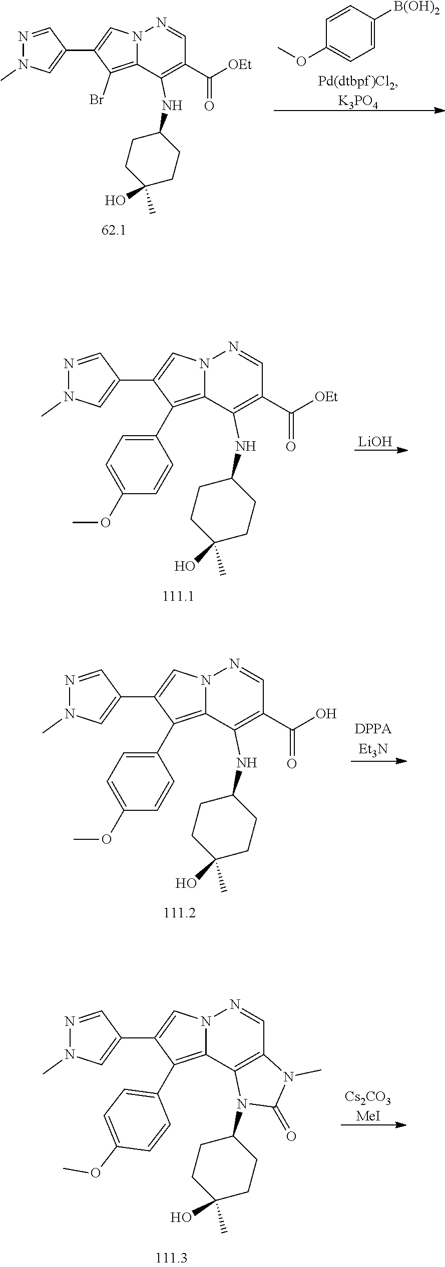

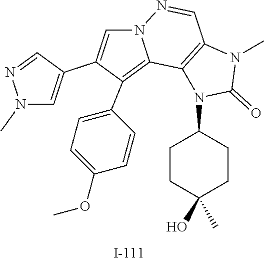

Synthesis of compound 111.1. Compound 111.1 was prepared from compound 62.1 and (4-methoxyphenyl)boronic acid following the procedure described in the synthesis of compound 59.12. The product was purified by flash column chromatography on silica gel (40% ethyl acetate in hexane). MS(ES): m/z 504.3 [M+H]⁺.

Synthesis of compound 111.2. Compound 111.2 was prepared from compound 111.1 following the procedure described in the synthesis of compound 1.10. The product was triturated with pentane, dried under vacuum and used in the next step without purification. MS(ES): m/z 476.31 [M+H]⁺.

Synthesis of compound 111.3. Compound 111.3 was prepared from compound 111.2 following the procedure described in the synthesis of compound 1.11. The product was purified by flash column chromatography on silica gel (Combiflash®, 1.6% methanol in DCM). MS(ES): m/z 473.2 [M+H]⁺.

Synthesis of compound I-111. Compound I-111 was prepared from compound 111.3 following the procedure described in the synthesis of compound 1.12. The product was purified by flash column chromatography on silica gel (1.2% methanol in DCM). MS(ES): m/z 487.20 [M+H]⁺, ¹H-NMR (400 MHz, DMSO-d₆): δ 8.36 (s, 1H), 8.12 (s, 1H), 7.28 (d, J=8.4 Hz, 2H), 7.16 (s, 1H), 7.08 (d, J=8.4 Hz, 2H), 6.97 (s, 1H), 3.85 (s, 3H), 3.70 (s, 3H), 3.40-3.28 (m, 4H), 2.58-2.56 (m, 2H), 1.32-1.29 (m, 3H), 1.05-1.03 (m, 2H), 0.93 (s, 3H), 0.60-0.54 (m, 2H).

### Example I-112: 3-((1S,3S)-3-(8-(3,6-dihydro-2H-pyran-4-yl)-3-methyl-9-(1-methyl-1H-indazol-5-yl)-2-oxo-2,3-dihydro-1H-imidazo[4,5-d]pyrrolo[1,2-b]pyridazin-1-yl)cyclopentyl)oxazolidin-2-one

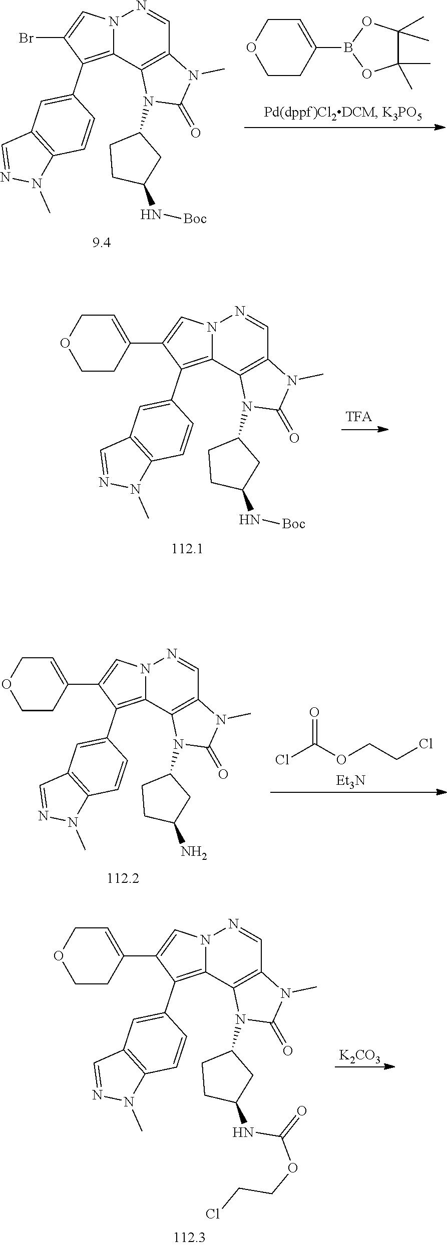

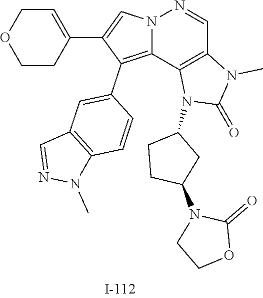 [SMILES: Cn1ncc2cc(-c3c(C4=CCOCC4)cn4ncc5c(c34)n([C@H]3CC[C@H](N4CCOC4=O)C3)c(=O)n5C)ccc21]

Synthesis of compound 112.1. Compound 112.1 was prepared from compound 9.4 and 2-(3,6-dihydro-2H-pyran-4-yl)-4,4,5,5-tetramethyl-1,3,2-dioxaborolane following the procedure described in the synthesis of compound 1.13. The product was purified by flash column chromatography on silica gel (2.4% methanol in DCM). MS(ES): m/z 584.6 [M+H]⁺.

Synthesis of compound 112.2. Compound 112.2 was prepared from compound 112.1 following the procedure described in the synthesis of compound 1.14. The product was used in the step without purification. MS(ES): m/z 484.3 [M+H]⁺.

Synthesis of compound 112.3. Compound 112.3 was prepared from compound 112.2 following the procedure described in the synthesis of compound 9.7. The product was purified by flash column chromatography on silica gel (Combiflash®, 2.8% methanol in DCM). MS(ES): m/z 591.5 [M+H]⁺.

Synthesis of I-112. Compound I-112 was prepared from compound 112.3 following the procedure described in the synthesis of compound I-9. The product was purified by flash column chromatography on silica gel (Combiflash®, 4% methanol in DCM). MS(ES): m/z 554.48 [M+H]⁺, ¹H-NMR (400 MHz, DMSO-d₆): δ 8.42 (s, 1H), 8.11 (d, J=10.4 Hz, 1H), 7.97 (s, 1H), 7.81 (d, J=12.4 Hz, 2H), 7.46-7.39 (m, 1H), 5.41 (s, 1H), 4.26-4.20 (m, 1H), 4.11 (s, 3H), 3.88 (s, 2H), 3.63 (s, 2H), 3.35 (s, 3H), 3.08-3.06 (m, 1H), 2.88-2.86 (m, 1H), 2.67-2.59 (m, 2H), 2.29 (s, 2H), 1.94-1.85 (m, 2H), 1.53 (s, 1H), 1.34 (s, 1H), 1.12-1.06 (m, 1H), 0.79-0.77 (m, 1H), 0.46 (s, 1H).

### Example I-113: 9-(benzo[d][1,3]dioxol-5-yl)-8-(2,3-dihydropyrazolo[5,1-b]oxazol-7-yl)-1-((1s,4s)-4-hydroxy-4-methylcyclohexyl)-3-methyl-1,3-dihydro-2H-imidazo[4,5-d]pyrrolo[1,2-b]pyridazin-2-one

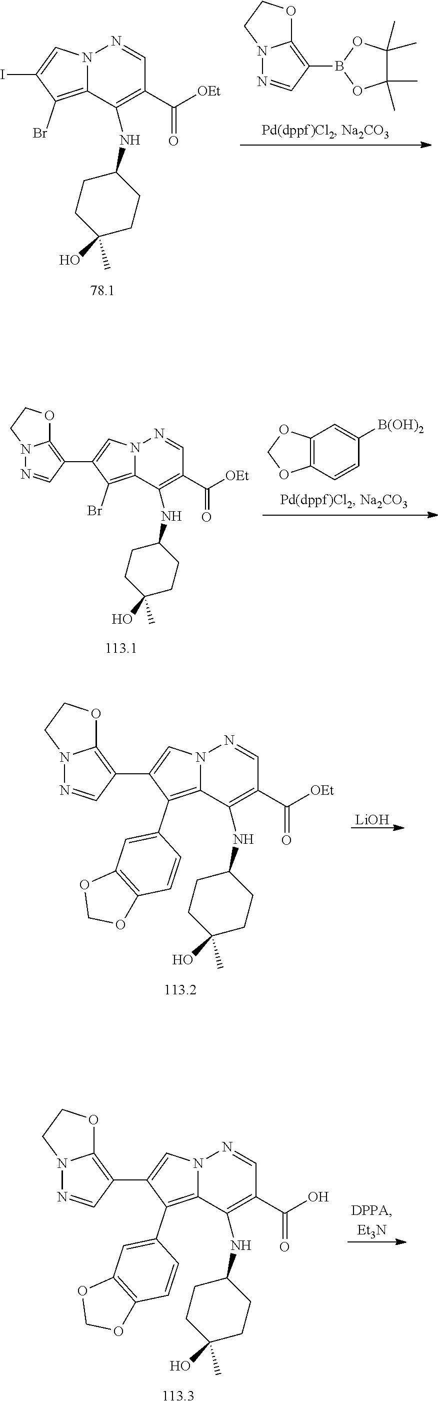

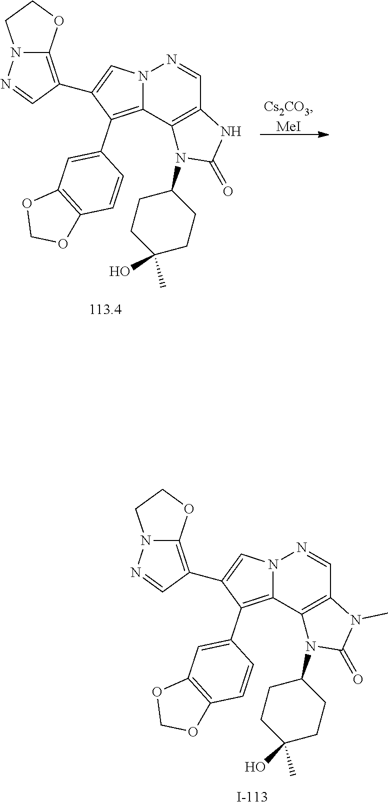

Synthesis of compound 113.1. Compound 113.1 was prepared from compound 9.4 and 7-(4,4,5,5-tetramethyl-1,3,2-dioxaborolan-2-yl)-2,3-dihydropyrazolo[5,1-b]oxazole following the procedure described in the synthesis of compound 1.13. The product was purified by flash column chromatography on silica gel (Combiflash®, 2.6% methanol in DCM). MS(ES): m/z 504.2 and 505.2 [M+H]⁺.

Synthesis of compound 113.2. Compound 113.2 was prepared from compound 113.1 and benzo[d][1,3]dioxol-5-ylboronic acid following the procedure described in the synthesis of compound 1.13. The product was purified by flash column chromatography on silica gel (Combiflash®, 2.6% methanol in dichloromethane). MS(ES): m/z 546.23 [M+H]⁺.

Synthesis of compound 113.3. Compound 113.3 was prepared from compound 113.2 following the procedure described in the synthesis of compound 1.10. The product was triturated with pentane, dried under vacuum and used in the next step without purification. MS(ES): m/z 518.20 [M+H]⁺.

Synthesis of compound 113.4. Compound 113.4 was prepared from compound 113.3 following the procedure described in the synthesis of compound 1.11. The product was purified by flash column chromatography on silica gel (Combiflash®, 3.1% methanol in DCM). MS(ES): m/z 515.10 [M+H]⁺.

Synthesis of compound I-113. Compound I-113 was prepared from compound 113.4 following the procedure described in the synthesis of compound 1.12. The product was purified by flash column chromatography on silica gel (3.2% methanol in DCM). MS(ES): m/z 529.28 [M+H]⁺, ¹H NMR (400 MHz, DMSO-d₆): δ 8.35 (s, 1H), 7.77 (s, 1H), 7.05 (d, J=7.6 Hz, 1H), 6.93 (s, 1H), 6.84 (d, J=1.6 Hz, 1H), 6.33 (s, 1H), 6.13 (s, 1H), 6.08 (s, 1H), 5.14-5.10 (m, 2H), 4.25-4.21 (m, 2H), 3.90 (s, 1H), 3.49 (m, 1H), 3.31 (s, 3H), 1.41-1.38 (m, 2H), 1.13-1.10 (m, 2H), 1.06-1.03 (m, 1H), 0.98 (s, 3H), 0.75-0.70 (m, 1H), 0.64-0.62 (m, 2H).

### Example I-114: 9-(benzo[d][1,3]dioxol-5-yl)-1-((1s,4s)-4-hydroxy-4-methylcyclohexyl)-3-methyl-8-(1-methyl-1H-pyrazol-4-yl)-1,3-dihydro-2H-imidazo[4,5-d]pyrrolo[1,2-b]pyridazin-2-one

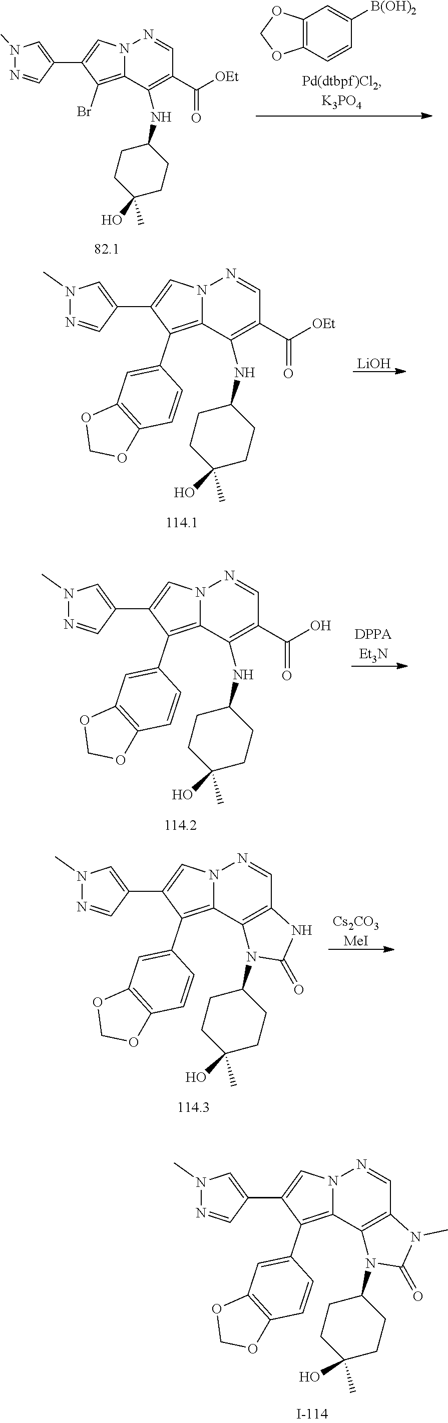

Synthesis of compound 114.1. Compound 114.1 was prepared from compound 82.1 and benzo[d][1,3]dioxol-5-ylboronic acid following the procedure described in the synthesis of compound 59.12. The product was purified by flash column chromatography on silica gel (40% ethyl acetate in hexane). MS(ES): m/z 518.6 [M+H]⁺.

Synthesis of compound 114.2. Compound 114.2 was prepared from compound 114.1 following the procedure described in the synthesis of compound 1.10. The product was triturated with pentane, dried under vacuum and used in the next step without purification. MS(ES): m/z 490.3 [M+H]⁺.

Synthesis of compound 114.3. Compound 114.3 was prepared from compound 114.2 following the procedure described in the synthesis of compound 1.11. The product was purified by flash column chromatography on silica gel (Combiflash®, 1.6% methanol in DCM). MS(ES): m/z 487.21 [M+H]⁺.

Synthesis of compound I-114. Compound I-114 was prepared from compound 114.3 following the procedure described in the synthesis of compound 1.12. The product was purified by flash column chromatography on silica gel (1.2% methanol in DCM). MS(ES): m/z 501.3 [M+H]⁺, ¹H-NMR (400 MHz, DMSO-d₆): δ 8.37 (s, 1H), 8.11 (s, 1H), 7.23 (s, 1H), 7.06 (d, J=7.6 Hz, 2H), 6.92 (d, J=1.6 Hz, 1H), 6.83 (dd, J=1.60, 8.0 Hz, 1H), 6.14 (s, 1H), 6.10 (s, 1H), 3.90 (s, 1H), 3.73 (s, 3H), 3.49-3.45 (m, 1H), 3.35 (s, 3H), 2.61-2.56 (m, 1H), 1.43-1.34 (m, 3H), 1.14-1.04 (m, 2H), 0.99 (m, 3H), 0.69-0.60 (m, 2H).

### Example I-115: 9-(2,3-dihydrobenzo[b][1,4]dioxin-6-yl)-8-(2,3-dihydropyrazolo[5,1-b]oxazol-7-yl)-1-((1s,4s)-4-hydroxy-4-methylcyclohexyl)-3-methyl-1,3-dihydro-2H-imidazo[4,5-d]pyrrolo[1,2-b]pyridazin-2-one

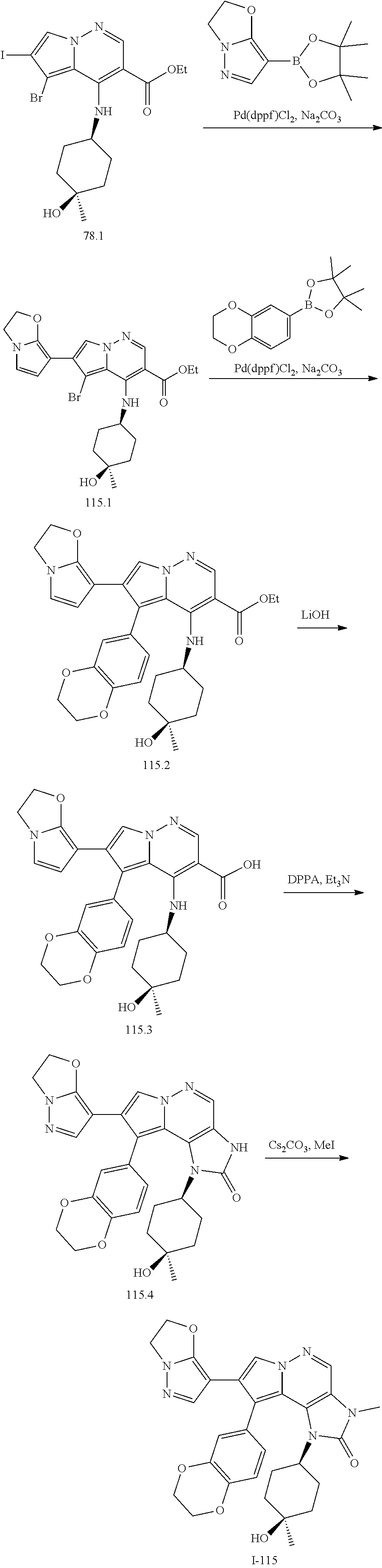

Synthesis of compound 115.1. Compound 115.1 was prepared from compound 78.1 and 7-(4,4,5,5-tetramethyl-1,3,2-dioxaborolan-2-yl)-2,3-dihydropyrazolo[5,1-b]oxazole following the procedure described in the synthesis of compound 1.13. The product was purified by flash column chromatography on silica gel (Combiflash®, 2.6% methanol in DCM). MS(ES): m/z 503.4 and 505.2 [M+H]⁺.

Synthesis of compound 115.2. Compound 115.2 was prepared from compound 115.1 and 2-(2,3-dihydrobenzo[b][1,4]dioxin-6-yl)-4,4,5,5-tetramethyl-1,3,2-dioxaborolane following the procedure described in the synthesis of compound 1.13. The product was purified by flash column chromatography on silica gel (3.2% methanol in DCM). MS(ES): m/z 559.25 [M+H]⁺.

Synthesis of compound 115.3. Compound 115.3 was prepared from compound 115.2 following the procedure described in the synthesis of compound 1.10. The product was triturated with pentane, dried under vacuum and used in the next step without purification. MS(ES): m/z 531.22 [M+H]⁺.

Synthesis of compound 115.4. Compound 115.4 was prepared from compound 115.3 following the procedure described in the synthesis of compound 1.11. The product was purified by flash column chromatography on silica gel (Combiflash®, 3.5% methanol in DCM). MS(ES): m/z 529.22 [M+H]⁺.

Synthesis of compound I-115. Compound I-115 was prepared from compound 115.4 following the procedure described in the synthesis of compound 1.12. The product was purified by flash column chromatography on silica gel (3.1% methanol in DCM). MS(ES): m/z 543.48 [M+H]⁺, ¹H-NMR (400 MHz, DMSO-d₆): δ 8.34 (s, 1H), 7.75 (s, 1H), 7.71 (d, J=8.0 Hz, 1H), 6.98 (d, J=8.4 Hz, 1H), 6.82 (s, 1H), 6.28 (s, 1H), 5.14-5.10 (m, 2H), 4.35-4.30 (m, 2H), 4.25-4.21 (m, 4H), 3.50-3.47 (m, 2H), 2.50 (s, 3H), 1.42-1.37 (m, 3H), 1.33-1.29 (m, 1H), 1.07-1.03 (m, 2H), 0.99 (s, 3H), 0.69-0.64 (m, 2H).

### Example I-116: 1-((1s,4s)-4-hydroxy-4-methylcyclohexyl)-3-methyl-9-(1-methyl-1H-indazol-5-yl)-8-(thiazol-5-yl)-1,3-dihydro-2H-imidazo[4,5-d]pyrrolo[1,2-b]pyridazin-2-one

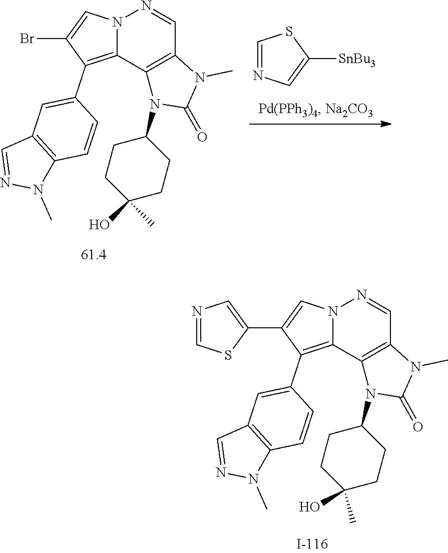

Synthesis of compound I-116. A mixture of 61.4 (0.085 g, 0.16 mmol, 1.0 equiv), 5-(tributylstannyl)thiazole (0.249 g, 0.66 mmol, 4.0 equiv) and sodium carbonate (0.054 g, 0.48 mmol, 3 equiv) in 1,4-dioxane (3 mL) was degassed by bubbling through a stream of argon for 10 min. Tetrakis(triphenylphosphine)palladium (0) (0.019 g, 0.016 mmol, 0.1 equiv) was added and degassed for 5 min. The reaction mixture was stirred at 140° C. for 1 h. It was cooled to room temperature, filtered through a pad of Celite® and rinsed with ethyl acetate. The filtrate was transferred into water and extracted with ethyl acetate. The combined organic layers were washed with brine, dried over anhydrous sodium sulfate, filtered and concentrated under reduced pressure. The residue was purified by flash column chromatography on silica gel (2.8% methanol in DCM) to afford I-116 (0.026 g, 30%). MS(ES): m/z 514.6 [M+H]⁺, ¹H-NMR (400 MHz, DMSO-d₆): δ 8.73 (s, 1H), 8.46 (d, J=16.0 Hz, 2H), 8.11 (s, 1H), 7.85-7.80 (m, 3H), 7.38-7.36 (m, 1H), 4.12 (s, 3H), 3.71 (m, 1H), 3.34 (s, 3H), 3.15 (m, 1H), 2.41-2.33 (m, 2H), 1.15-1.12 (m, 1H), 0.96-0.93 (m, 4H), 0.53 (s, 3H), 0.10-0.04 (m, 1H).

### Example I-117: 9-(6,7-dihydro-4H-thieno[3,2-c]pyran-2-yl)-1-((1s,4s)-4-hydroxy-4-methylcyclohexyl)-8-(3-methoxy-1-methyl-1H-pyrazol-4-yl)-3-methyl-1,3-dihydro-2H-imidazo[4,5-d]pyrrolo[1,2-b]pyridazin-2-one

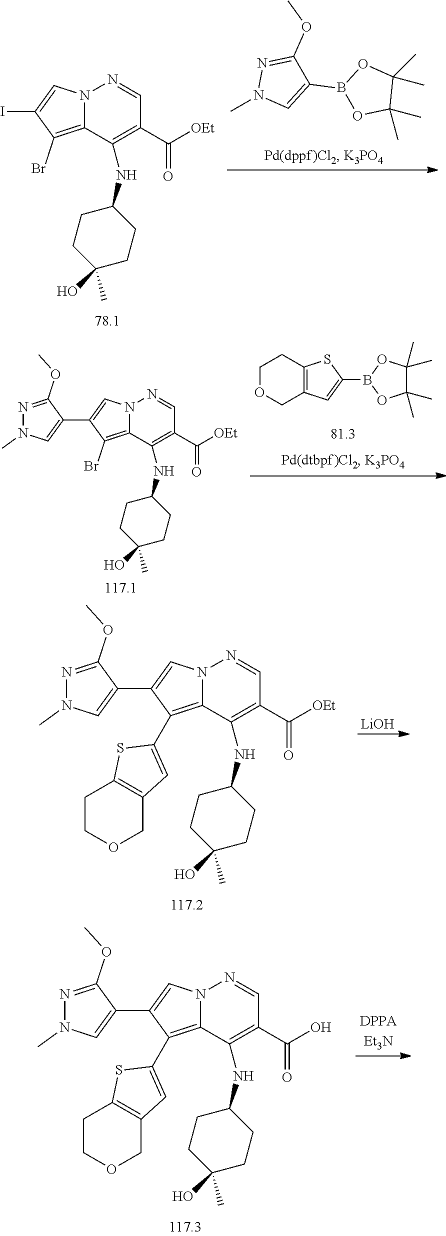

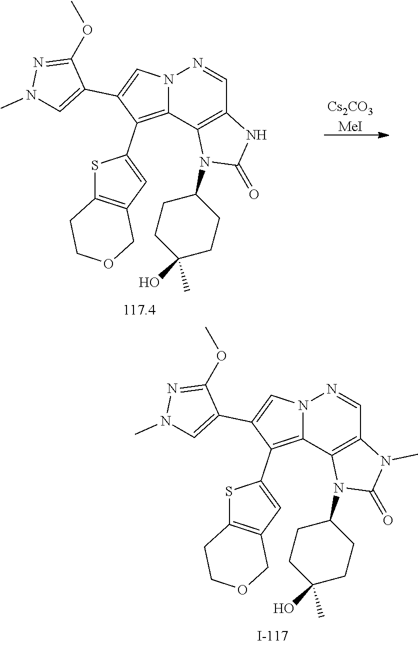

Synthesis of compound 117.1. Compound 117.1 was prepared from compound 78.1 and 3-methoxy-1-methyl-4-(4,4,5,5-tetramethyl-1,3,2-dioxaborolan-2-yl)-1H-pyrazole following the procedure described in the synthesis of compound 54.3. The product was purified by flash column chromatography on silica gel (55% ethyl acetate in hexane). MS(ES): m/z 507.52 & 509.52 [M+H]⁺.

Synthesis of compound 117.2. Compound 117.2 was prepared from compound 117.1 and 81.3 following the procedure described in the synthesis of compound 59.12. The product was purified by flash column chromatography on silica gel (55% ethyl acetate in hexane). MS(ES): m/z 566.73 [M+H]⁺.

Synthesis of compound 117.3. Compound 117.3 was prepared from compound 117.2 following the procedure described in the synthesis of compound 1.10. The product was triturated with pentane, dried under vacuum and used in the next step without purification. MS(ES): m/z 538.68 [M+H]⁺.

Synthesis of compound 117.4. Compound 117.4 was prepared from compound 117.3 following the procedure described in the synthesis of compound 1.11. The product was purified by flash column chromatography on silica gel (Combiflash®, 2.1% methanol in DCM). MS(ES): m/z 535.67 [M+H]⁺.

Synthesis of compound I-117. Compound I-117 was prepared from compound 117.4 following the procedure described in the synthesis of compound 1.12. The product was purified by flash column chromatography on silica gel (1.8% methanol in DCM). MS (ES): m/z 549.65 [M+H]⁺, ¹H-NMR (400 MHz, DMSO-d₆): δ 8.42 (s, 1H), 8.02 (s, 1H), 6.84 (s, 1H), 6.45 (s, 1H), 4.77-4.73 (m, 1H), 4.60-4.56 (m, 1H), 3.98-3.99 (m, 2H), 3.93 (s, 1H), 3.90 (s, 3H), 3.73-3.70 (m, 1H), 3.59 (s, 3H), 3.36 (s, 3H), 2.99-2.98 (m, 1H), 2.83-2.79 (m, 2H), 2.68 (s, 1H) 1.48-1.45 (m, 2H), 1.24-1.20 (m, 2H), 1.10 (s, 3H), 0.86 (t, J=3.6 Hz, 2H).

### Example I-118: methyl ((1R,3R)-3-(9-(benzo[d]oxazol-6-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

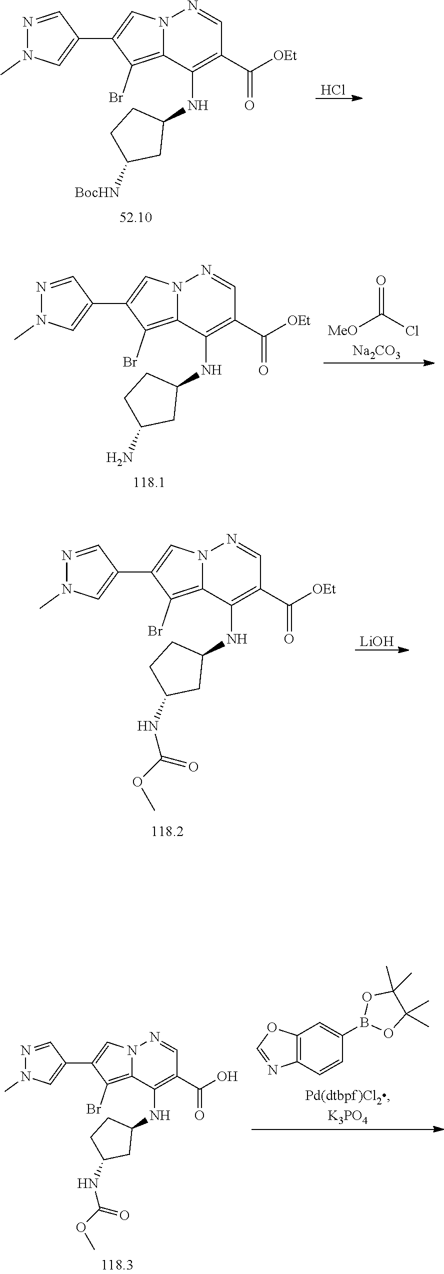

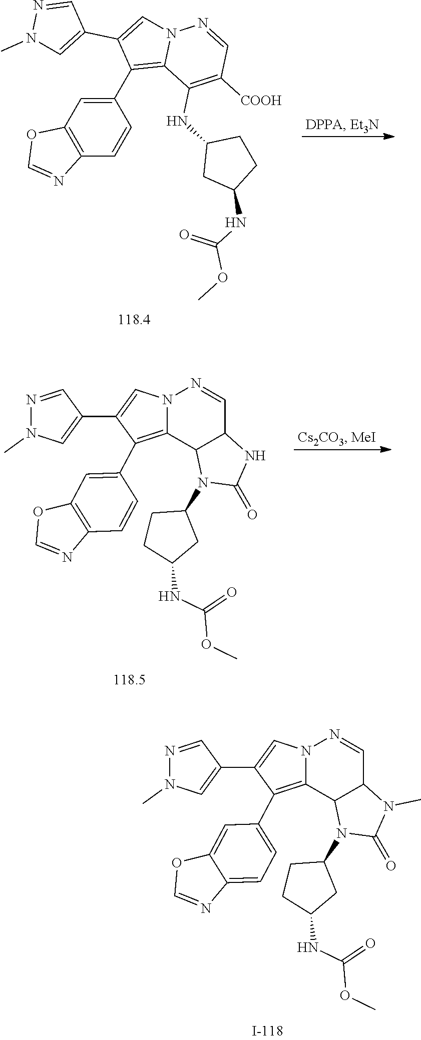

Synthesis of compound 118.1. To a solution of 52.10 (0.400 g, 0.730 mmol, 1.0 equiv) in DCM (5 mL) was added 4 N HCl in dioxane (1.5 mL) dropwise at 0° C. The reaction mixture was stirred at room temperature for 1 h. It was transferred to ice-water to neutralize with sodium bicarbonate and extracted with DCM. The combined organic layers were washed with brine, dried over anhydrous sodium sulfate, filtered and concentrated under reduced pressure to afford 118.1 (0.310 g, 95%). MS (ES): m/z 447.2 and 449.3 [M+H]⁺.

Synthesis of compound 118.2. Compound 118.2 was prepared from compound 118.1 following the procedure described in the synthesis of compound I-1. The product was purified by flash column chromatography on silica gel (Combiflash®, 1.1% methanol in DCM). MS(ES): m/z 505.6 and 507.5 [M+H]⁺.

Synthesis of compound 118.3. Compound 118.3 was prepared from compound 118.2 following the procedure described in the synthesis of compound 1.10. The product was used in the next step without purification. MS(ES): m/z 477.5 and 479.3 [M+H]⁺.

Synthesis of compound 118.4. Compound 118.4 was prepared from compound 118.3 and 6-(4,4,5,5-tetramethyl-1,3,2-dioxaborolan-2-yl)benzo[d]oxazole following the procedure described in the synthesis of compound 59.12. The product was used in the next step without purification. MS (ES): m/z 516.4 [M+H]⁺.

Synthesis of compound 118.5. Compound 118.5 was prepared from compound 118.4 following the procedure described in the synthesis of compound 1.11. The product was purified by flash column chromatography on silica gel (2.1% methanol in DCM). MS (ES): m/z 513.5 [M+H]⁺.

Synthesis of I-118. Compound I-118 was prepared from compound 118.5 following the procedure described in the synthesis of compound 1.12. The product was purified by flash column chromatography on silica gel (2.6% methanol in DCM). MS(ES): m/z 527.2 [M+H]⁺, ¹H-NMR (400 MHz, DMSO-d₆): δ 8.82 (s, 1H), 8.32 (s, 1H), 7.89-7.88 (m, 2H), 7.60 (s, 1H), 7.48-7.43 (m, 2H), 7.35 (s, 1H), 7.02 (s, 1H), 5.21-5.17 (m, 1H), 4.30-4.29 (m, 1H), 3.77 (s, 3H), 3.56 (s, 3H), 3.37 (s, 3H), 2.33-2.18 (m, 3H), 1.96 (m, 1H), 1.65 (m, 1H), 1.23-0.85 (m, 1H).

### Example I-119: methyl ((1R,3R)-3-(9-(5-cyclopropyl-6-oxo-1,6-dihydropyridazin-3-yl)-8-(3-methoxy-1-methyl-1H-pyrazol-4-yl)-3-methyl-2-oxo-2,3-dihydro-1H-imidazo[4,5-d]pyrrolo[1,2-b]pyridazin-1-yl)cyclopentyl)carbamate

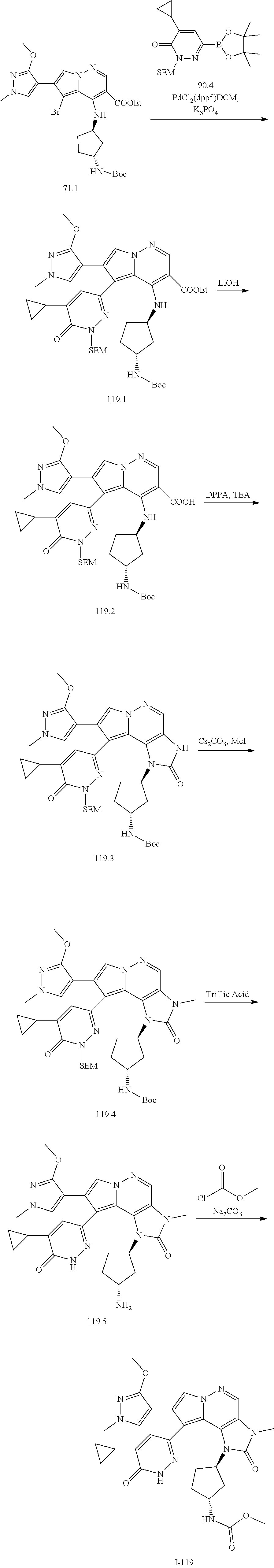

Synthesis of compound 119.1. Compound 119.1 was prepared from compound 71.1 and 90.4 following the procedure described in the synthesis of compound 54.3. The product was purified by flash column chromatography on silica gel (2.5% methanol in DCM). MS(ES): m/z 763.39 [M+H]⁺.

Synthesis of compound 119.2. Compound 119.2 was prepared from compound 119.1 following the procedure described in the synthesis of compound 1.10. The product was triturated with pentane, dried under vacuum and used in the next step without purification. MS(ES): m/z 735.36 [M+H]⁺.

Synthesis of compound 119.3. Compound 119.3 was prepared from compound 119.2 following the procedure described in the synthesis of compound 1.11. The product was purified by flash column chromatography on silica gel (Combiflash®, 3.2% methanol in DCM). MS(ES): m/z 732.36 [M+H]⁺.

Synthesis of compound 119.4. Compound 119.4 was prepared from compound 119.3 following the procedure described in the synthesis of compound 1.12. The product was purified by flash column chromatography on silica gel (3.2% methanol in DCM). MS(ES): m/z 746.38 [M+H]⁺.

Synthesis of compound 119.5. Compound 119.5 was prepared from compound 119.4 following the procedure described in the synthesis of compound 21.8. The product was triturated with n-pentane, dried under reduced pressure and used in the nest step without purification. MS(ES): m/z 516.24 [M+H]⁺.

Synthesis of compound I-119. Compound I-119 was prepared from compound 119.5 following the procedure described in the synthesis of compound I-1 The product was purified by flash column chromatography on silica gel (Combiflash®, 4% methanol in DCM). MS (ES): m/z 574.25 [M+H]⁺, ¹H-NMR (400 MHz, DMSO-d₆): δ 12.82 (s, 1H), 8.37 (s, 1H), 8.02 (s, 1H), 7.00 (m, 1H), 6.84 (s, 1H), 6.43 (s, 1H), 4.37-4.35 (m, 3H), 4.32 (m, 1H), 4.28 (m, 1H), 3.90 (s, 3H), 3.56 (s, 3H), 3.52 (s, 3H), 3.38 (s, 3H), 2.01 (m, 1H), 1.91-1.90 (m, 2H), 1.37-1.35 (m, 1H), 1.25-1.24 (m, 2H), 0.87-0.85 (m, 2H).

### Example I-120: 9-(6,7-dihydro-4H-thieno[3,2-c]pyran-2-yl)-8-(3-fluoro-1-methyl-1H-pyrazol-4-yl)-1-((1s,4s)-4-hydroxy-4-methylcyclohexyl)-3-methyl-1,3-dihydro-2H-imidazo[4,5-d]pyrrolo[1,2-b]pyridazin-2-one

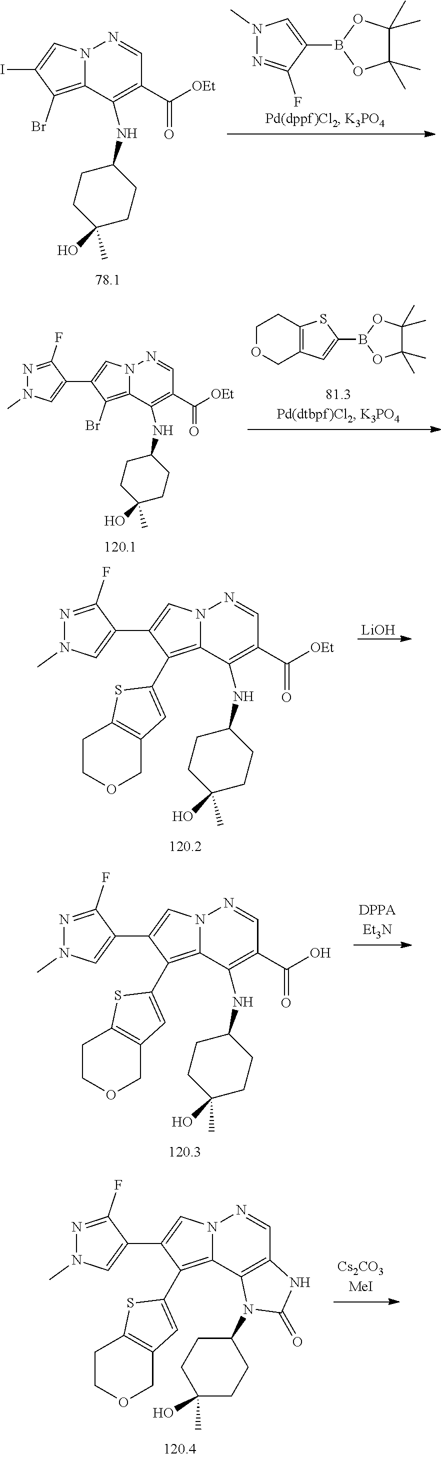

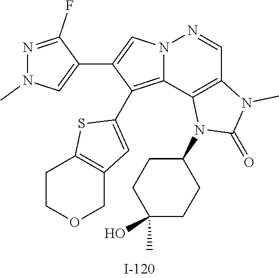

Synthesis of compound 120.1. Compound 120.1 was prepared from compound 78.1 and 3-fluoro-1-methyl-4-(4,4,5,5-tetramethyl-1,3,2-dioxaborolan-2-yl)-1H-pyrazole following the procedure described in the synthesis of compound 54.3. The product was purified by flash column chromatography on silica gel (55% ethyl acetate in hexane). MS(ES): m/z 494.52 & 496.52 [M+H]⁺.

Synthesis of compound 120.2. Compound 120.2 was prepared from compound 120.1 and 81.3 following the procedure described in the synthesis of compound 54.3. The product was purified by flash column chromatography on silica gel (55% ethyl acetate in hexane). MS(ES): m/z 554.73 [M+H]⁺.

Synthesis of compound 120.3. Compound 120.3 was prepared from compound 120.2 following the procedure described in the synthesis of compound 1.10. The product was triturated with pentane, dried under vacuum and used in the next step without purification. MS(ES): m/z 526.68 [M+H]⁺.

Synthesis of compound 120.4. Compound 120.4 was prepared from compound 120.3 following the procedure described in the synthesis of compound 1.11. The product was purified by flash column chromatography on silica gel (Combiflash®, 2.1% methanol in DCM). MS(ES): m/z 523.67 [M+H]⁺.

Synthesis of compound I-120. Compound I-120 was prepared from compound 120.4 following the procedure described in the synthesis of compound 1.12. The product was purified by flash column chromatography on silica gel (1.8% methanol in DCM). MS (ES): m/z 537.65 [M+H]⁺, ¹H-NMR (400 MHz, DMSO-d₆): δ 8.59 (s, 1H), 8.27 (s, 1H), 6.86 (s, 1H), 5.66 (d, J=6.0 Hz, 1H), 4.57 (bs, 2H), 3.95-3.93 (m, 3H), 3.64 (s, 3H), 3.60-3.57 (m, 1H), 3.40 (s, 3H), 2.82 (bs, 2H), 2.69 (s, 2H), 1.46 (d, J=12.4 Hz, 2H), 1.20 (d, J=9.6 Hz, 2H), 1.05 (s, 3H), 0.82 (t, J=12.8 Hz, 2H).

### Example I-121: 1-((1s,4s)-4-hydroxy-4-methylcyclohexyl)-8-(3-methoxy-1-methyl-1H-pyrazol-4-yl)-3-methyl-9-(thieno[2,3-c]pyridin-2-yl)-1,3-dihydro-2H-imidazo[4,5-d]pyrrolo[1,2-b]pyridazin-2-one

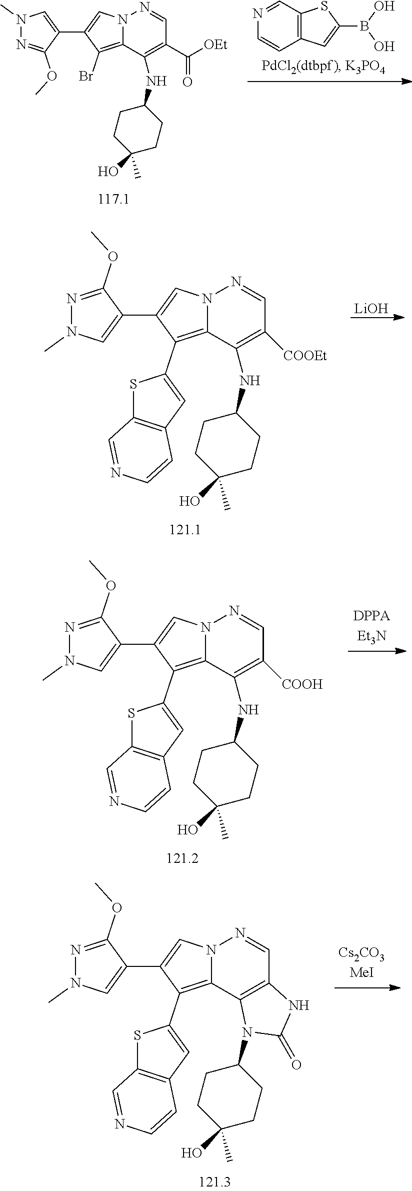 [SMILES: CCOC(=O)c1cnn2cc(-c3cn(C)nc3OC)c(Br)c2c1N[C@H]1CC[C@@](C)(O)CC1 ; OB(O)c1cc2ccncc2s1 ; CCOC(=O)c1cnn2cc(-c3cn(C)nc3OC)c(-c3cc4ccncc4s3)c2c1N[C@H]1CC[C@@](C)(O)CC1 ; "LiOH" ; COc1nn(C)cc1-c1cn2ncc(C(=O)O)c(N[C@H]3CC[C@@](C)(O)CC3)c2c1-c1cc2ccncc2s1 ; COc1nn(C)cc1-c1cn2ncc3[nH]c(=O)n([C@H]4CC[C@@](C)(O)CC4)c3c2c1-c1cc2ccncc2s1]

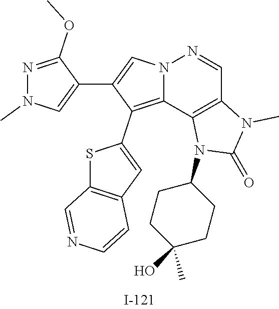

Synthesis of compound 121.1. Compound 121.1 was prepared from compound 117.1 and thieno[2,3-c]pyridin-2-ylboronic acid following the procedure described in the synthesis of compound 59.12. The product was purified by flash column chromatography on silica gel (55% ethyl acetate in hexane). MS(ES): m/z 561.62 [M+H]⁺.

Synthesis of compound 121.2. Compound 121.2 was prepared from compound 121.1 following the procedure described in the synthesis of compound 1.10. The product was triturated with pentane, dried under vacuum and used in the next step without purification. MS(ES): m/z 533.68 [M+H]⁺.

Synthesis of compound 121.3. Compound 121.3 was prepared from compound 121.2 following the procedure described in the synthesis of compound 1.11. The product was purified by flash column chromatography on silica gel (Combiflash®, 2.1% methanol in DCM). MS(ES): m/z 530.67 [M+H]⁺.

Synthesis of compound I-121. Compound I-121 was prepared from compound 121.3 following the procedure described in the synthesis of compound 1.12. The product was purified by flash column chromatography on silica gel (1.8% methanol in DCM). MS (ES): m/z 544.65 [M+H]⁺, ¹H-NMR (400 MHz, DMSO-d₆): δ 9.31 (s, 1H), 8.57 (d, J=5.6 Hz, 1H), 8.51 (s, 1H), 8.09 (s, 1H), 7.90 (d, J=5.2 Hz, 1H), 7.64 (s, 1H), 6.64 (s, 1H), 3.87 (s, 3H), 3.76 (s, 1H), 3.49 (s, 3H), 3.41 (s, 3H), 2.61 (s, 3H), 1.13-1.11 (m, 4H), 0.46 (s, 3H).

### Example I-122: methyl ((1R,3R)-3-(9-(3,4-dihydro-1H-pyrrolo[2,1-c][1,4]oxazin-7-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

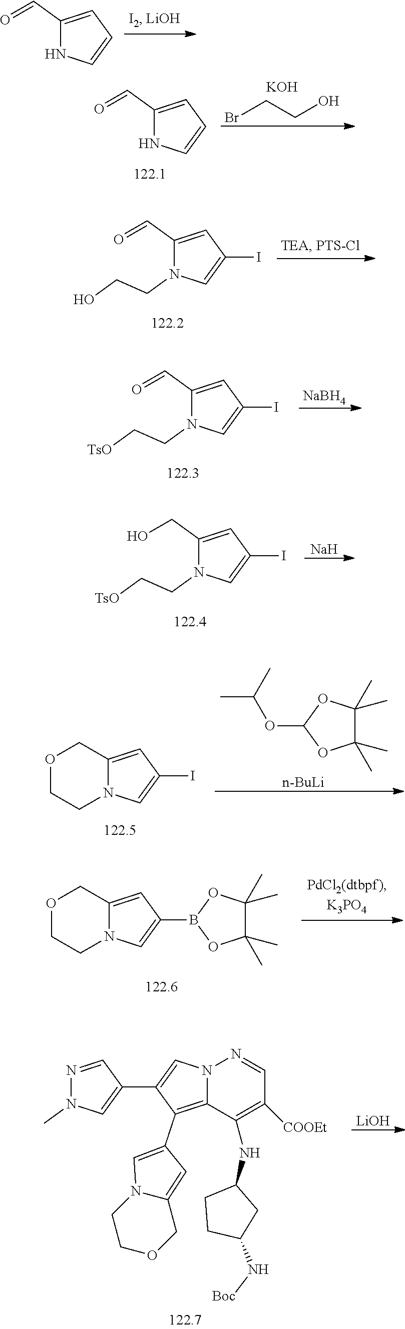

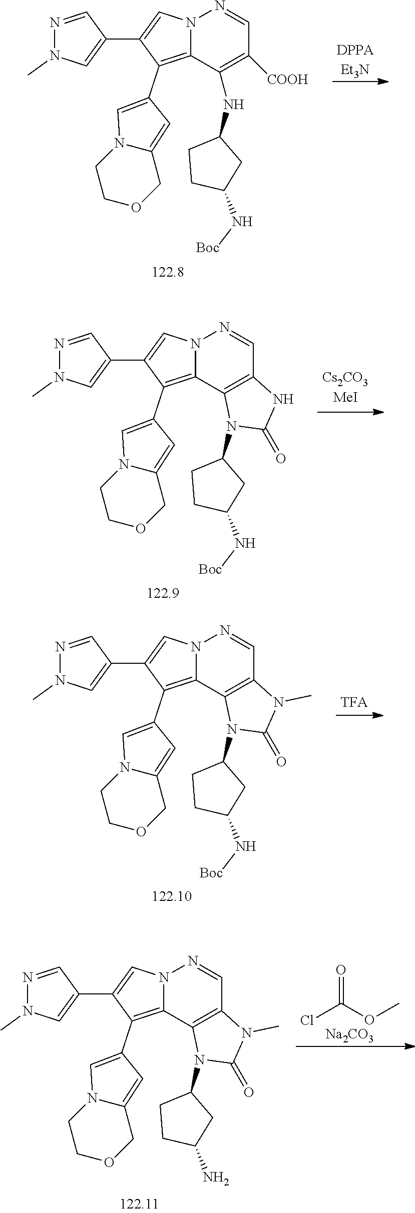

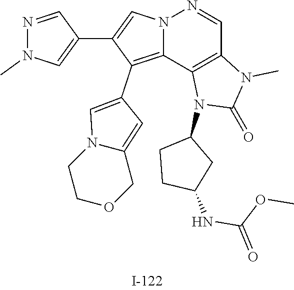

Synthesis of compound 122.1. To a solution of lithium hydroxide (17.68 g, 0.4210 mol, 4.0 equiv) and 1H-pyrrole-2-carbaldehyde (10 g, 0.1052 mol, 1 equiv) in DMF (180 mL) at −5° C. was added iodine (13.7 g, 0.11 mol, 1.0 equiv) in DMF (140 mL) and stirred for 2 h. The reaction mixture was filtered and rinsed with water and ethyl acetate. The aqueous phase was extracted with ethyl acetate. The combined organic layers were washed with water twice and once with saturated brine, dried and concentrated under reduced pressure. The residue was purified by flash column chromatography on silica gel (15% ethyl acetate in hexane) to afford 122.1 (14.0 g, 60%). MS(ES): m/z 222.1 [M+H]⁺.

Synthesis of compound 122.2. To a solution of potassium hydroxide (3.5 g, 1.90 mmol, 1.0 equiv) and 122.1 (14.0 g, 63.35 mmol, 33 equiv) in dioxane (20 mL) was added 2-2-bromoethan-1-ol (9.50 g, 76.02 mmol, 1.2 equiv) and stirred at 60° C. for 16 h. The reaction mixture was cooled to room temperature and the pH was adjusted to ˜6 using acetic acid. It was quenched with ice-water and extract with ethyl acetate. The combined organic layers were washed with brine, dried over anhydrous sodium sulfate, filtered and concentrated under reduced pressure. The residue was purified by flash column chromatography on silica gel (60% ethyl acetate in hexane) to afford 122.2 (11.7 g, 70%). MS(ES): m/z 265 [M+H]⁺.

Synthesis of compound 122.3. To a solution of triethylamine (18.4 mL, 13.2 mmol, 3 equiv) and 122.2 (11.7 g, 44.4 mmol, 1.0 equiv) in DCM (250 mL) at 0° C. was added 4-methyl-benzenesulfonyl chloride (12.5 g, 6.51 mmol, 1.5 equiv) over 15 min. The reaction mixture was allowed to warm to ambient temperature and stirred overnight. To the mixture was added water and extracted with DCM. The combined organic layers were washed with saturated sodium bicarbonate solution, brine, dried over anhydrous sodium sulfate, filtered and concentrated under reduced pressure. The residue was purified by flash column chromatography on silica gel (22% ethyl acetate in hexane) to afford 122.3 (6 g, 32%). MS(ES): m/z 420.1 [M+H]⁺.

Synthesis of compound 122.4 To a solution of 122.3 (6.0 g, 14.31 mmol, 1.5 equiv) in ethanol (25 mL) was added sodium borohydride (95 mg, 2.50 mmol) at 0° C. and stirred for 30 min. The reaction mixture was allowed to warm to ambient temperature and stirred for 1 h. The reaction was quenched with acetic acid and concentrated under reduced pressure. The residue was partitioned between water and DCM and the organic phase was washed with water, brine, dried over anhydrous sodium sulfate, filtered and concentrated under reduced pressure to afford 122.4 (3.7 g, 61%). MS(ES): m/z 422.1 [M+H]⁺.

Synthesis of compound 122.5. To a solution of 122.4 (3.7 g, 7.83 mmol, 1.0 equiv) in THF (50 mL) was added sodium hydride (0.50 g, 1.05 mmol, 1.2 equiv) at 0° C. The reaction mixture was stirred at room temperature for 30 min. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (1.2% methanol in DCM) to afford 122.5 (1.10 g, 56%). MS(ES): m/z 250.0 [M+H]⁺.

Synthesis of compound 122.6. To a solution of 122.5 (1.10 g, 4.42 mmol, 1.0 equiv) in THF (100 mL) was added n-BuLi (2.5 M in hexane, 0.17 mL, 1.0 equiv) dropwise at −78° C. and stirred for 30 min. To the mixture was added isopropoxyboronic acid pinacol ester (0.99 g, 5.30 mmol, 1.2 equiv). The reaction was allowed to warm to room temperature and stirred for 1 h. It was quenched with a saturated aqueous solution of NH₄C₁, extracted with DCM, dried over anhydrous sodium sulfate, filtered and concentrated under reduced pressure. The residue was purified by flash column chromatography on silica gel (20% ethyl acetate in hexane) to afford 122.6 (0.460 g, 42%). MS(ES): m/z 250.0 [M+H]⁺.

Synthesis of compound 122.7. Compound 121.7 was prepared from compound 122.6 and 52.10 following the procedure described in the synthesis of compound 59.12. The product was purified by flash column chromatography on silica gel (Combiflash®, 45% ethyl acetate in hexane). MS(ES): m/z 590.8 [M+H]⁺.

Synthesis of compound 122.8. Compound 122.8 was prepared from compound 122.7 following the procedure described in the synthesis of compound 1.10. The product was triturated with pentane, dried under vacuum and used in the next step without purification. MS(ES): m/z 562.6 [M+H]⁺.

Synthesis of compound 122.9. Compound 122.9 was prepared from compound 122.8 following the procedure described in the synthesis of compound 1.11. The product was purified by flash column chromatography on silica gel (Combiflash®, 1.6% methanol in DCM). MS(ES): m/z 559.6 [M+H]⁺.

Synthesis of compound 122.10. Compound 122.10 was prepared from compound 122.9 following the procedure described in the synthesis of compound 1.12. The product was purified by flash column chromatography on silica gel (1.2% methanol in DCM). MS(ES): m/z 573.6 [M+H]⁺.

Synthesis of compound 122.11. Compound 122.11 was prepared from compound 122.10 following the procedure described in the synthesis of compound 1.14. The product was triturated with pentane and dried under reduced pressure. It was used in the next step without purification. MS(ES): m/z 473.5 [M+H]⁺.

Synthesis of compound I-122. Compound I-122 was prepared from compound 122.11 following the procedure described in the synthesis of compound I-1. The product was purified by flash column chromatography on silica gel (Combiflash®, 3.8% methanol in DCM). MS(ES): m/z 531.4 [M+H]⁺, ¹H-NMR (400 MHz, DMSO-d₆): δ 8.31 (s, 1H), 8.06 (s, 1H), 7.49 (s, 1H), 7.35 (s, 1H), 7.08 (s, 1H), 6.75 (s, 1H), 5.84 (s, 1H), 4.88 (m, 3H), 4.32-4.30 (m, 1H), 4.08-4.06 (m, 4H), 3.77 (s, 3H), 3.58 (s, 3H), 3.38 (s, 3H), 2.12 (m, 1H), 2.00-1.94 (m, 2H), 1.59 (m, 1H), 1.30-1.29 (m, 1H), 1.09-1.08 (m, 1H).

### Example I-123: 9-(6,7-dihydro-4H-thieno[3,2-c]pyran-2-yl)-1-((1s,4s)-4-hydroxy-4-methylcyclohexyl)-3-methyl-8-(1-methyl-1H-pyrazol-4-yl)-1,3-dihydro-2H-imidazo[4,5-d]pyrrolo[1,2-b]pyridazin-2-one

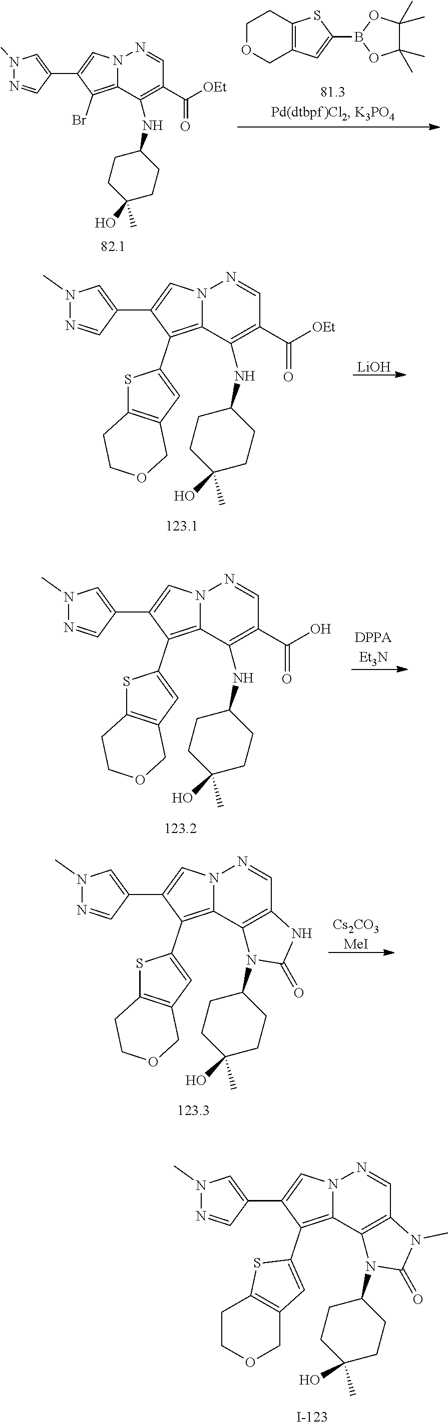

Synthesis of compound 123.1. Compound 123.1 was prepared from compound 82.1 and 81.3 following the procedure described in the synthesis of compound 59.12. The product was purified by flash column chromatography on silica gel (40% ethyl acetate in hexane). MS(ES): m/z 536.6 [M+H]⁺.

Synthesis of compound 123.2. Compound 123.2 was prepared from compound 123.1 following the procedure described in the synthesis of compound 1.10. The product was triturated with pentane, dried under vacuum and used in the next step without purification. MS(ES): m/z 508.6 [M+H]⁺.

Synthesis of compound 123.3. Compound 123.3 was prepared from compound 123.2 following the procedure described in the synthesis of compound 1.11. The product was purified by flash column chromatography on silica gel (Combiflash®, 1.6% methanol in DCM). MS(ES): m/z 505.7 [M+H]⁺.

Synthesis of compound I-123. Compound I-123 was prepared from compound 123.3 following the procedure described in the synthesis of compound 1.12. The product was purified by flash column chromatography on silica gel (1.2% methanol in DCM). MS(ES): m/z 519.2 [M+H]⁺, ¹H-NMR (400 MHz, DMSO-d₆): δ 8.42 (s, 1H), 8.12 (s, 1H), 7.42 (s, 1H), 7.12 (s, 1H), 6.85 (s, 1H), 4.74-4.54 (m, 1H), 3.97 (bs, 2H), 3.92 (s, 1H), 3.77 (s, 3H), 3.70-3.67 (m, 1H), 3.33 (s, 3H), 2.93 (m, 1H), 2.76-2.73 (m, 1H), 2.67-2.60 (m, 1H), 1.47-1.44 (m, 2H), 1.23-1.19 (m, 3H), 1.05 (s, 3H), 0.84 (t, J=12.8 Hz, 3H).

### Example I-124: 9-(benzo[d][1,3]dioxol-5-yl)-1-((1s,4s)-4-hydroxy-4-methylcyclohexyl)-3-methyl-8-(thiazol-5-yl)-1,3-dihydro-2H-imidazo[4,5-d]pyrrolo[1,2-b]pyridazin-2-one

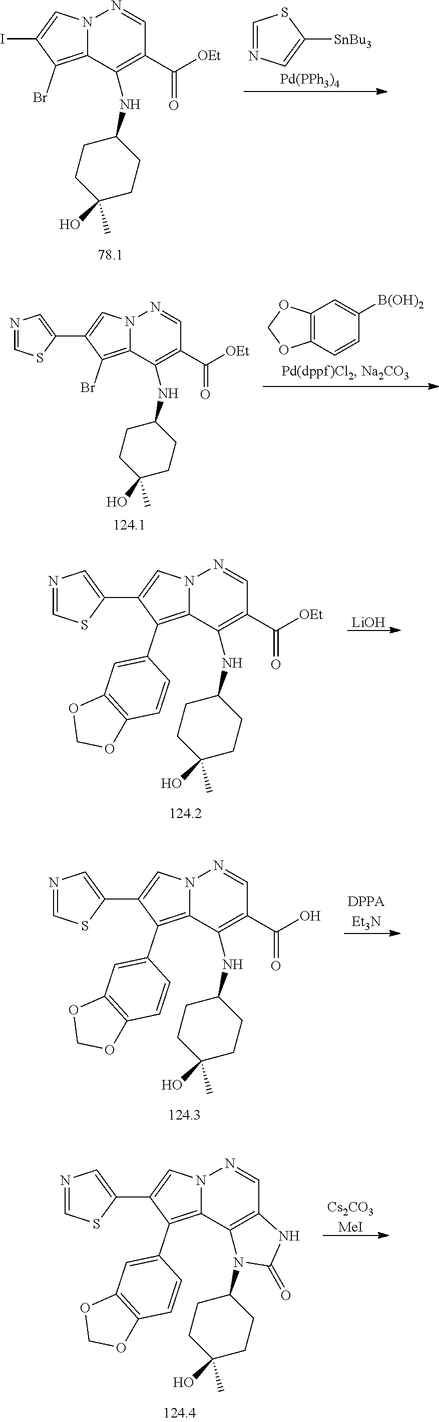

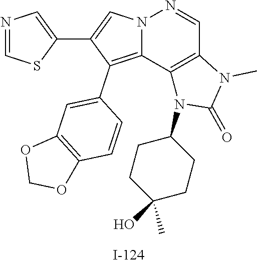 [SMILES: Cn1c(=O)n([C@H]2CC[C@@](C)(O)CC2)c2c1cnn1cc(-c3cncs3)c(-c3ccc4c(c3)OCO4)c21]

Synthesis of compound 124.1. Compound 124.1 was prepared from compound 78.1 and 5-(tributylstannyl)thiazole following the procedure described in the synthesis of compound I-116. The product was purified by flash column chromatography on silica gel (Combiflash®, 1.9% methanol in DCM). MS(ES): m/z 479.2 & 481.2 [M+H]⁺.

Synthesis of compound 124.2. Compound 124.2 was prepared from compound 124.1 and benzo[d][1,3]dioxol-5-ylboronic acid following the procedure described in the synthesis of compound 1.13. The product was purified by flash column chromatography on silica gel (60% ethyl acetate in hexane). MS(ES): m/z 521.18 [M+H]⁺.

Synthesis of compound 124.3. Compound 124.3 was prepared from compound 124.2 following the procedure described in the synthesis of compound 1.10. The product was triturated with pentane, dried under vacuum and used in the next step without purification. MS(ES): m/z 493.05 [M+H]⁺.

Synthesis of compound 124.4. Compound 124.4 was prepared from compound 124.3 following the procedure described in the synthesis of compound 1.11. The product was purified by flash column chromatography on silica gel (Combiflash®, 2.9% methanol in DCM). MS(ES): m/z 490.35 [M+H]⁺.

Synthesis of compound I-124. Compound I-124 was prepared from compound 124.4 following the procedure described in the synthesis of compound 1.12. The product was purified by flash column chromatography on silica gel (1.8% methanol in DCM). MS (ES): m/z 504.3 [M+H]⁺, ¹H-NMR (400 MHz, DMSO-d₆): δ 8.86 (s, 1H), 8.48 (s, 1H), 8.40 (s, 1H), 7.90 (s, 1H), 7.08 (d, J=8.0 Hz, 1H), 6.98 (s, 1H), 6.87 (d, J=6.8 Hz, 1H), 6.17 (s, 1H), 6.11 (s, 1H), 3.93 (s, 1H), 3.52-3.46 (m, 1H), 3.37 (s, 3H), 2.64-2.62 (m, 1H), 1.44-1.35 (m, 3H), 1.16-1.13 (m, 1H), 1.08-1.06 (m, 1H), 1.00 (s, 3H), 0.65-0.64 (m, 2H).

### Example I-125: (1R,2R)-2-fluoro-N-((1s,4S)-1-methyl-4-(3-methyl-9-(1-methyl-1H-indazol-5-yl)-2-oxo-8-(thiazol-2-yl)-2,3-dihydro-1H-imidazo[4,5-d]pyrrolo[1,2-b]pyridazin-1-yl)cyclohexyl)cyclopropane-1-carboxamide

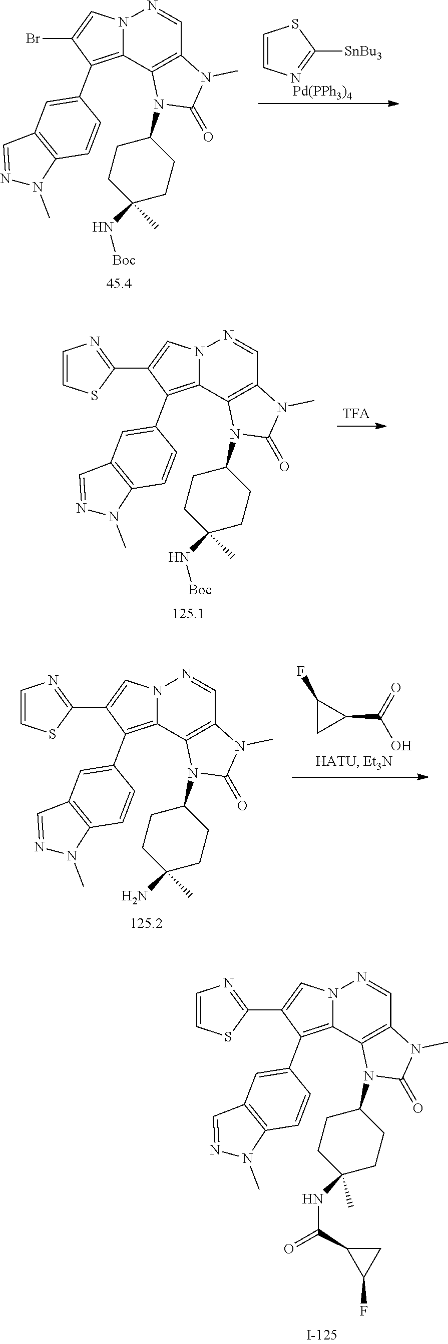

Synthesis of compound 125.1. Compound 125.1 was prepared from compound 45.4 and 2-(tributylstannyl)thiazole following the procedure described in the synthesis of compound I-116. The product was purified by flash column chromatography on silica gel (Combiflash®, 0-2.8% methanol in DCM). MS(ES): m/z 613.4 [M+H]⁺.

Synthesis of compound 125.2. Compound 125.2 was prepared from compound 125.1 following the procedure described in the synthesis of compound 1.14. The product was triturated with pentane and dried under reduced pressure. It was used in the next step without purification. MS(ES): m/z 513.4 [M+H]⁺.

Synthesis of I-125. Compound I-125 was prepared from compound 125.2 and (1R,2R)-2-fluorocyclopropane-1-carboxylic acid following the procedure described in the synthesis of compound I-45. The product was purified by flash column chromatography on silica gel (Combiflash®, 0-2.4% methanol in DCM). MS(ES): m/z 599.3 [M+H]⁺, ¹H NMR (DMSO-d₆, 400 MHz): δ 8.74 (d, J=0.4 Hz, 1H), 8.49 (s, 1H), 8.45 (s, 1H), 8.14 (s, 1H), 7.87 (s, 1H), 7.86 (s, 1H), 7.84 (s, 1H), 7.40 (dd, J=1.20, 8.6 Hz, 1H), 7.29 (s, 1H), 4.78 (s, 1H), 4.14 (s, 3H), 3.38 (s, 3H), 3.28 (m, 1H), 2.46 (m, 2H), 1.79 (m, 3H), 1.42 (m, 1H), 1.36 (m, 1H), 1.02 (m, 2H), 0.66 (s, 3H), −0.074 (t, J=63.6 Hz, 1H), −0.568 (t, J=Hz, 1H).

### Example I-126: methyl ((1R,3R)-3-(8-(3-methoxy-1-methyl-1H-pyrazol-4-yl)-3-methyl-2-oxo-9-(6-oxo-5-(trifluoromethyl)-1,6-dihydropyridazin-3-yl)-2,3-dihydro-1H-imidazo[4,5-d]pyrrolo[1,2-b]pyridazin-1-yl)cyclopentyl)carbamate

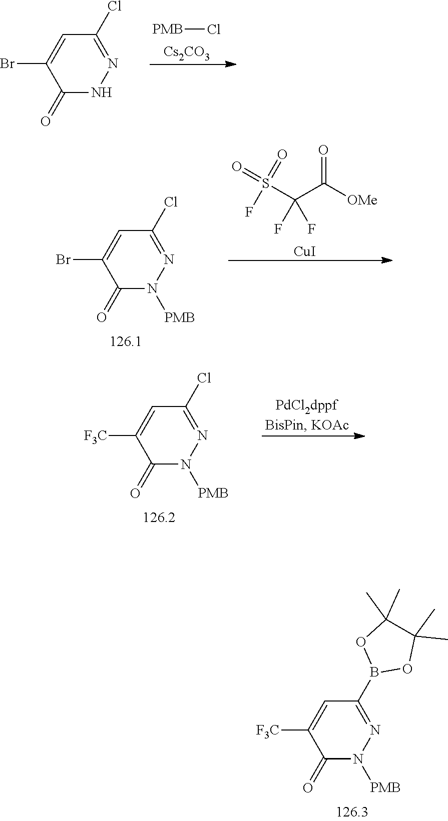

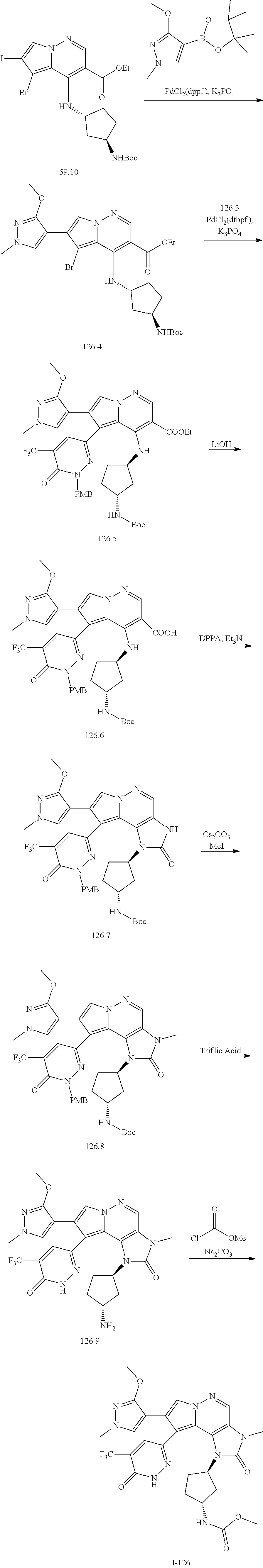

Synthesis of compound 126.1. To a stirred solution of 4-bromo-6-chloropyridazin-3(2H)-one (5.0 g, 23.87 mmol, 1.0 equiv) in DMF (60 mL) was added cesium carbonate (23.32 g, 71.77 mmol, 3.0 equiv) and 4-methoxybenzyl chloride (5.6 g, 35.88 mmol, 1.5 equiv) at 0° C. The reaction mixture was stirred at room temperature for 1 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (18% ethyl acetate in hexane) to afford 126.1 (4.2 g, 53%). MS (ES): m/z 330.5 and m/z 331.5 [M+H]⁺.

Synthesis of compound 126.2. A mixture of 126.1 (4.2 g, 12.74 mmol, 1.0 equiv), methyl 2,2-difluoro-2-(fluorosulfonyl)acetate (7.34 g, 38.23 mmol, 3.0 equiv) and copper (I) iodide (2.43 g, 12.74 mmol, 1.0 equiv) in DMF (50 mL) was stirred at 110° C. for 1 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (12% ethyl acetate in hexane) to afford 126.2 (3.9 g, 96%). MS (ES): m/z 319.6 [M+H]⁺.

Synthesis of compound 126.3. A mixture of 126.2 (3.9 g, 12.24 mmol, 1.0 equiv), bis(pinacolato)diboron (18.61 g, 73.58 mmol, 6.0 equiv) and potassium acetate (3.60 g, 36.79 mmol, 3.0 equiv) in dioxane (50 mL) was degassed by bubbling through a stream of argon for 10 min. [1,1′-Bis(diphenylphosphino)ferrocene]dichloropalladium(II) (0.896 mg, 1.22 mmol, 0.1 equiv) was added and degassed for 5 min. The reaction mixture was stirred at 80° C. for 1 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (25% ethyl acetate in hexane) to afford 126.3 (3.10 g, 62%). MS (ES): m/z 411.2 [M+H]⁺.

Synthesis of compound 126.4. Compound 126.4 was prepared from compound 59.10 and 3-methoxy-1-methyl-4-(4,4,5,5-tetramethyl-1,3,2-dioxaborolan-2-yl)-1H-pyrazole following the procedure described in the synthesis of compound 54.3. The product was purified by flash column chromatography on silica gel (55% ethyl acetate in hexane). MS(ES): m/z 577.52 and 579.52 [M+H]⁺.

Synthesis of compound 126.5. Compound 126.5 was prepared from compound 126.3 and 126.4 following the procedure described in the synthesis of compound 59.12. The product was purified by flash column chromatography on silica gel (55% ethyl acetate in hexane). MS(ES): m/z 781.62 [M+H]⁺.

Synthesis of compound 126.6. Compound 126.6 was prepared from compound 126.5 following the procedure described in the synthesis of compound 1.10. The product was triturated with pentane, dried under vacuum and used in the next step without purification. MS(ES): m/z 753.68 [M+H]⁺.

Synthesis of compound 126.7. Compound 126.7 was prepared from compound 126.6 following the procedure described in the synthesis of compound 1.11. The product was purified by flash column chromatography on silica gel (Combiflash®, 2.1% methanol in DCM). MS(ES): m/z 750.67 [M+H]⁺.

Synthesis of compound 126.8. Compound 126.8 was prepared from compound 126.7 following the procedure described in the synthesis of compound 1.12. The product was purified by flash column chromatography on silica gel (1.8% methanol in DCM). MS(ES): m/z 764.70 [M+H]⁺.

Synthesis of compound 126.9. Compound 126.9 was prepared from compound 126.8 following the procedure described in the synthesis of compound 21.8. The product was used in the next step without purification. MS(ES): m/z 544.58 [M+H]⁺.

Synthesis of compound I-126. Compound I-126 was prepared from compound 126.9 following the procedure described in the synthesis of compound I-1. The product was purified by flash column chromatography on silica gel (Combiflash®, 3.2% methanol in DCM). MS (ES): m/z 602.65 [M+H]⁺, ¹H-NMR (400 MHz, DMSO-d₆): δ 13.85 (s, 1H), 8.53 (s, 1H), 7.98 (s, 2H), 7.42 (s, 1H), 7.08 (s, 1H), 4.14 (s, 1H), 3.70 (d, J=9.6 Hz, 6H), 3.50 (s, 3H), 3.42 (s, 3H), 3.37 (s, 1H), 2.35-2.29 (m, 1H), 2.13 (bs, 1H), 2.01-1.99 (m, 1H), 1.761 (bs, 1H), 1.52-1.45 (m, 1H), 1.36 (s, 1H).

### Example I-127: 3-((1S,3S)-3-(9-(2,3-dihydrobenzo[b][1,4]dioxin-6-yl)-3-methyl-2-oxo-8-(thiazol-5-yl)-2,3-dihydro-1H-imidazo[4,5-d]pyrrolo[1,2-b]pyridazin-1-yl)cyclopentyl)oxazolidin-2-one

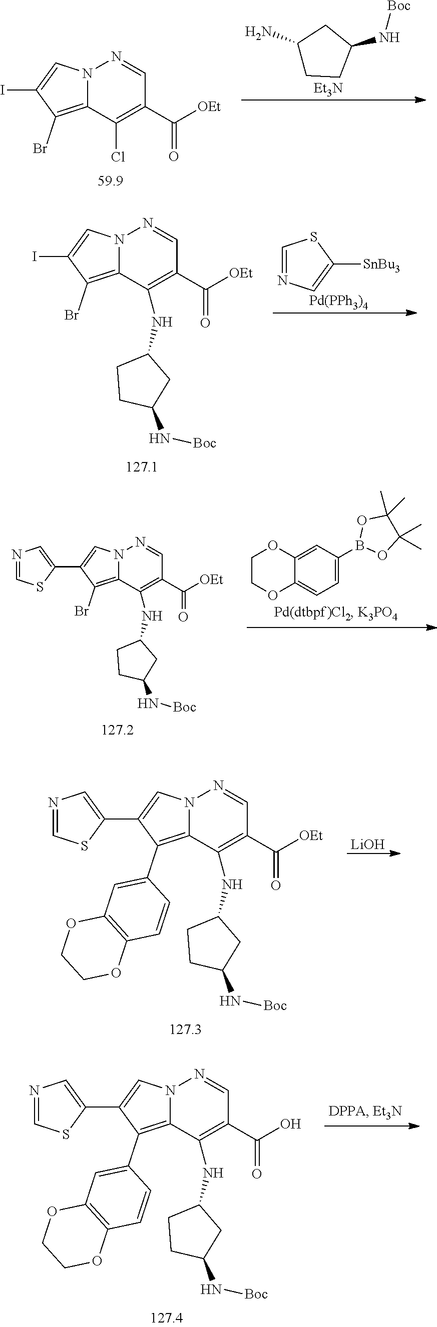

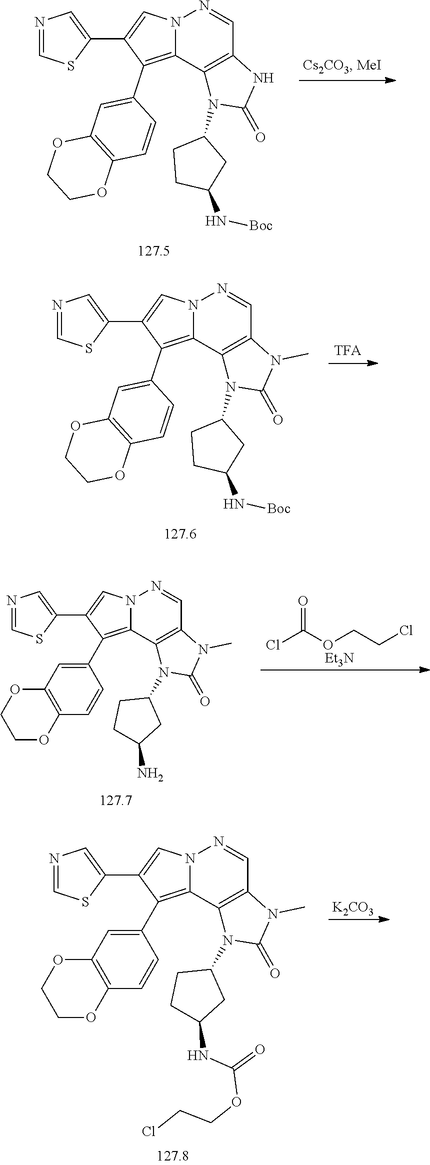

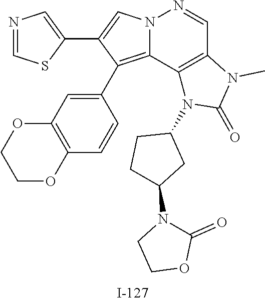 [SMILES: Cn1c(=O)n([C@H]2CC[C@H](N3CCOC3=O)C2)c2c1cnn1cc(-c3cncs3)c(-c3ccc4c(c3)OCCO4)c21]

Synthesis of compound 127.1. Compound 127.1 was prepared from compound 59.9 and tert-butyl ((1S,3S)-3-aminocyclopentyl)carbamate following the procedure described in the synthesis of compound 1.9. The product was purified by flash column chromatography on silica gel (22% ethyl acetate in n-hexane), MS (ES): m/z 593.1 and 595.1 [M+H]⁺.

Synthesis of compound 127.2. Compound 127.2 was prepared from compound 127.1 and 5-(tributylstannyl)thiazole following the procedure described in the synthesis of compound I-116. The product was purified by flash column chromatography on silica gel (Combiflash®, 42% ethyl acetate in n-hexane). MS(ES): m/z 550.6 and 552.5 [M+H]⁺.

Synthesis of compound 127.3. Compound 127.3 was prepared from compound 127.2 and 2-(2,3-dihydrobenzo[b][1,4]dioxin-6-yl)-4,4,5,5-tetramethyl-1,3,2-dioxaborolane following the procedure described in the synthesis of compound 59.12. The product was purified by flash column chromatography on silica gel (3.0% methanol in DCM). MS(ES): m/z 606.71 [M+H]⁺.

Synthesis of compound 127.4. Compound 127.4 was prepared from compound 127.3 following the procedure described in the synthesis of compound 1.10. The product was triturated with pentane, dried under vacuum and used in the next step without purification. MS(ES): m/z 578.66 [M+H]⁺.

Synthesis of compound 127.5. Compound 127.5 was prepared from compound 127.4 following the procedure described in the synthesis of compound 1.11. The product was purified by flash column chromatography on silica gel (Combiflash®, 5.0% methanol in DCM). MS(ES): m/z 575.66 [M+H]⁺.

Synthesis of compound 127.6. Compound 127.6 was prepared from compound 127.5 following the procedure described in the synthesis of compound 1.12. The product was purified by flash column chromatography on silica gel (3.5% methanol in DCM). MS(ES): m/z 589.68 [M+H]⁺.

Synthesis of compound 127.7. Compound 127.7 was prepared from compound 127.6 following the procedure described in the synthesis of compound 1.14. The product was triturated with n-pentane and dried under reduced pressure. It was used in the next step without purification. MS(ES): m/z 489.57 [M+H]⁺.

Synthesis of compound 127.8. Compound 127.8 was prepared from compound 127.7 and 2-chloroethyl carbonochloridate following the procedure described in the synthesis of compound 9.7. The product was purified by flash column chromatography on silica gel (2.0% methanol in DCM). MS(ES): m/z 596.07 [M+H]⁺.

Synthesis of compound I-127. Compound I-127 was prepared from compound 127.8 following the procedure described in the synthesis of compound I-9. The product was purified by flash column chromatography on silica gel (Combiflash®, 3.2% methanol in DCM). MS (ES): m/z 559.26 [M+H]⁺, ¹H-NMR (400 MHz, DMSO-d₆): δ 8.82 (s, 1H), 8.40 (s, 1H), 8.34 (s, 1H), 7.86 (d, J=12.0 Hz, 1H), 7.06 (d, J=8.8 Hz, 1H), 6.88-6.87 (m, 2H), 4.31-4.25 (m, 6H), 3.38 (m, 5H), 2.04 (m, 2H), 1.78 (m, 1H), 1.49-1.47 (m, 2H), 1.39-1.36 (m, 1H), 0.72-0.68 (m, 2H).

### Example I-128: 3-((1S,3S)-3-(9-(benzo[d][1,3]dioxol-5-yl)-8-(5,6-dihydro-4H-pyrrolo[1,2-b]pyrazol-3-yl)-3-methyl-2-oxo-2,3-dihydro-1H-imidazo[4,5-d]pyrrolo[1,2-b]pyridazin-1-yl)cyclopentyl)oxazolidin-2-one

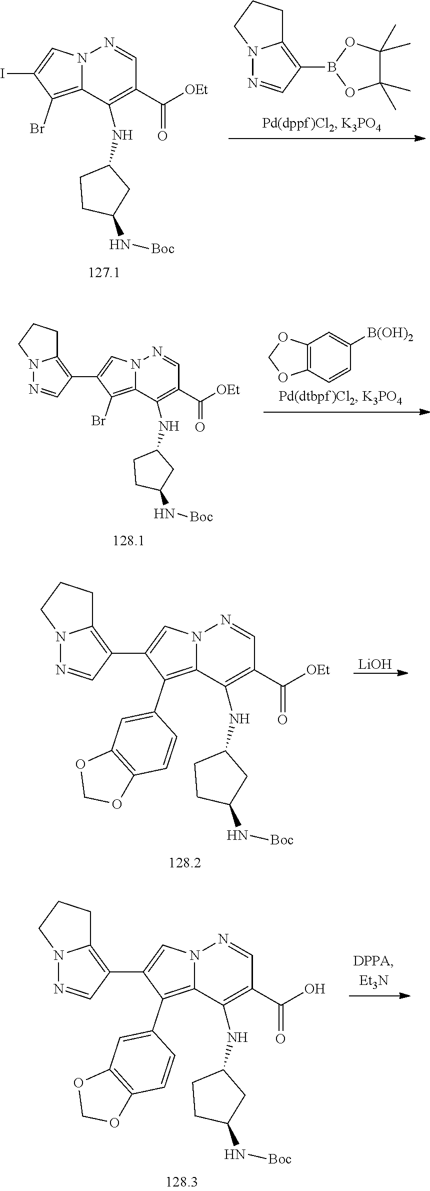

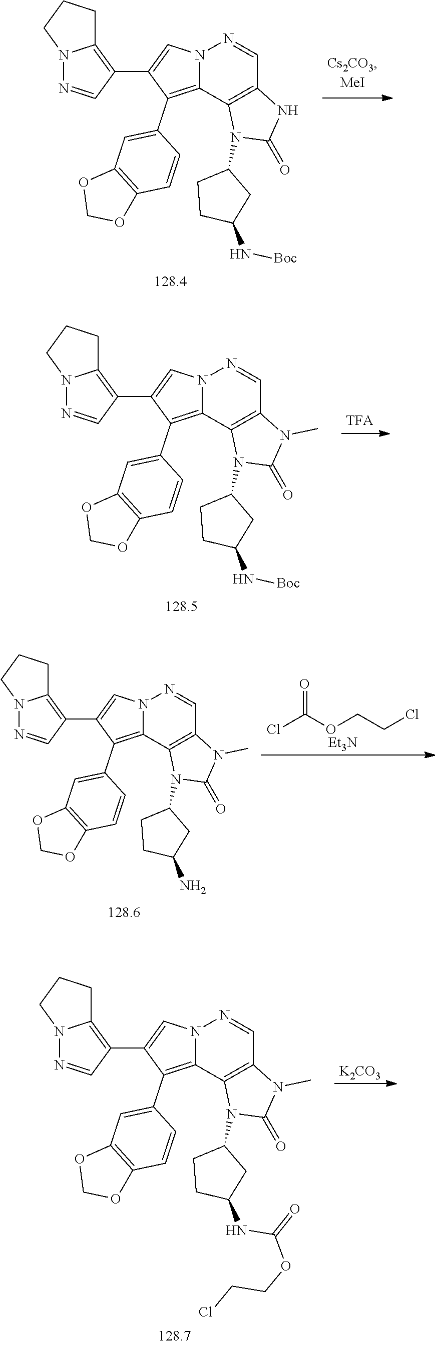

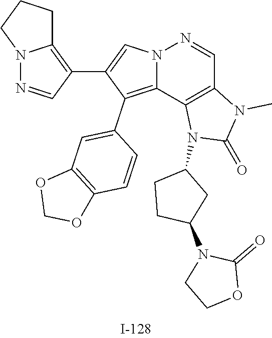 [SMILES: Cn1c(=O)n([C@H]2CC[C@H](N3CCOC3=O)C2)c2c1cnn1cc(-c3cnn4c3CCC4)c(-c3ccc4c(c3)OCO4)c21]

Synthesis of compound 128.1. Compound 128.1 was prepared from compound 127.1 and 3-(4,4,5,5-tetramethyl-1,3,2-dioxaborolan-2-yl)-5,6-dihydro-4H-pyrrolo[1,2-b]pyrazole following the procedure described in the synthesis of compound 54.3. The product was purified by flash column chromatography on silica gel (55% ethyl acetate in hexane). MS(ES): m/z 573.52 and 575.52 [M+H]⁺.

Synthesis of compound 128.2. Compound 128.2 was prepared from compound 128.1 and benzo[d][1,3]dioxol-5-ylboronic acid following the procedure described in the synthesis of compound 59.12. The product was purified by flash column chromatography on silica gel (55% ethyl acetate in hexane). MS(ES): m/z 615.70 [M+H]⁺.

Synthesis of compound 128.3. Compound 128.3 was prepared from compound 128.2 following the procedure described in the synthesis of compound 1.10. The product was triturated with pentane, dried under vacuum and used in the next step without purification. MS(ES): m/z 587.34 [M+H]⁺.

Synthesis of compound 128.4. Compound 128.4 was prepared from compound 128.3 following the procedure described in the synthesis of compound 1.11. The product was purified by flash column chromatography on silica gel (Combiflash®, 2.1% methanol in DCM). MS(ES): m/z 584.5 [M+H]⁺.

Synthesis of compound 128.5. Compound 128.5 was prepared from compound 128.4 following the procedure described in the synthesis of compound 1.12. The product was purified by flash column chromatography on silica gel (1.8% methanol in DCM). MS(ES): m/z 598.70 [M+H]⁺.

Synthesis of compound 128.6. Compound 128.6 was prepared from compound 128.5 following the procedure described in the synthesis of compound 1.14. The product was used in the next step without purification. MS(ES): m/z 498.3 [M+H]⁺.

Synthesis of compound 128.7. Compound 128.6 was prepared from compound 128.5 and 2-chloroethyl carbonochloridate following the procedure described in the synthesis of compound 9.7. The product was purified by flash column chromatography on silica gel (Combiflash®, 3.5% methanol in DCM). MS(ES) 605.65 [M+H]⁺.

Synthesis of I-128. Compound I-128 was prepared from compound 128.7 and following the procedure described in the synthesis of compound I-9. The product was purified by flash column chromatography on silica gel (Combiflash®, 3.2% methanol in DCM). MS(ES) 568.61 [M+H]⁺, ¹H-NMR (400 MHz, DMSO-d₆): δ 8.40 (s, 1H), 7.94 (s, 1H), 7.10 (dd, J=8.00, 21.4 Hz, 1H), 6.99 (d, J=1.6 Hz, 1H), 6.88 (t, J=1.6 Hz, 1H), 6.78 (d, J=19.2 Hz, 1H), 6.16-6.09 (m, 2H), 4.34-4.25 (m, 3H), 4.01 (t, J=7.2 Hz, 2H), 3.95 (s, 1H), 3.37 (s, 3H), 3.34 (s, 2H), 2.78-2.74 (m, 2H), 2.50 (s, 1H), 2.08 (s, 2H), 1.76 (s, 1H), 1.53-1.50 (m, 1H), 1.44-1.40 (m, 1H), 1.00-0.99 (m, 1H).

### Example I-129: 3-((1S,3S)-3-(9-(4-methoxyphenyl)-3-methyl-2-oxo-8-(thiazol-5-yl)-2,3-dihydro-1H-imidazo[4,5-d]pyrrolo[1,2-b]pyridazin-1-yl)cyclopentyl)oxazolidin-2-one

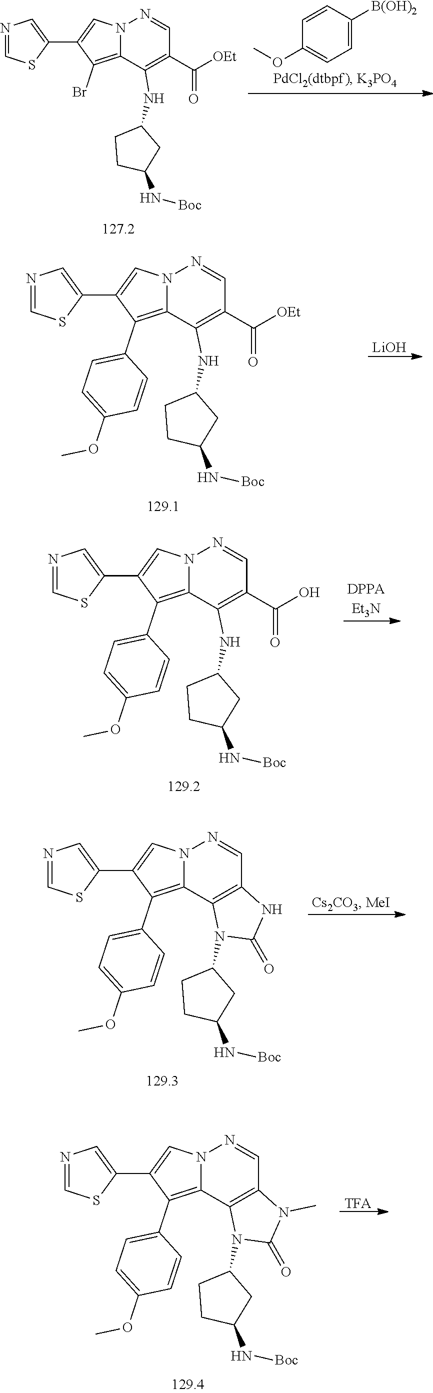

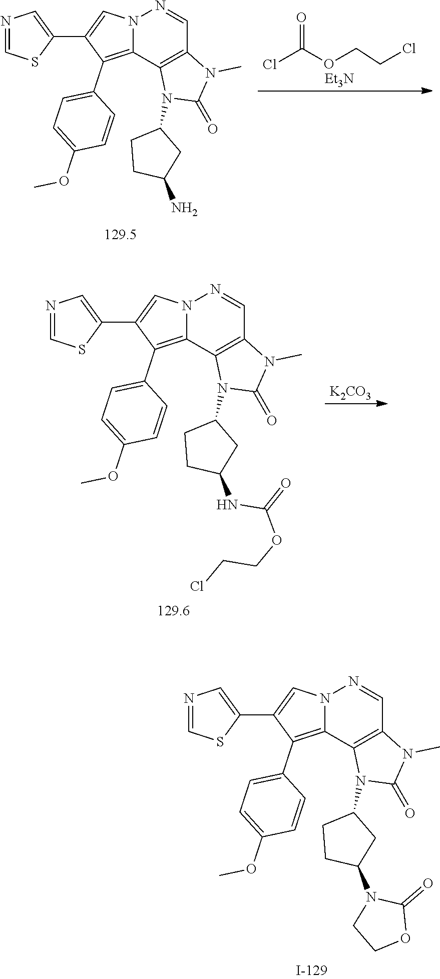

Synthesis of compound 129.1. Compound 129.1 was prepared from compound 127.2 and (4-methoxyphenyl)boronic acid following the procedure described in the synthesis of compound 59.12. The product was purified by flash column chromatography on silica gel (63% ethyl acetate in n-hexane). MS (ES): m/z 578.6 [M+H]⁺.

Synthesis of compound 129.2. Compound 129.2 was prepared from compound 129.1 following the procedure described in the synthesis of compound 1.10. The product was triturated with pentane, dried under vacuum and used in the next step without purification. MS(ES): m/z 550.4 [M+H]⁺.

Synthesis of compound 129.3. Compound 129.3 was prepared from compound 129.2 following the procedure described in the synthesis of compound 1.11. The product was purified by flash column chromatography on silica gel (Combiflash®, 2.0% methanol in DCM). MS(ES): m/z 547.5 [M+H]⁺.

Synthesis of compound 129.4. Compound 129.4 was prepared from compound 129.3 following the procedure described in the synthesis of compound 1.12. The product was purified by flash column chromatography on silica gel (1.8% methanol in DCM). MS(ES): m/z 561.3 [M+H]⁺.

Synthesis of compound 129.5. Compound 129.5 was prepared from compound 129.4 following the procedure described in the synthesis of compound 1.14. The product was used in the next step without purification. MS(ES): 461.7 [M+H]⁺.

Synthesis of compound 129.6. Compound 129.6 was prepared from compound 129.5 and 2-chloroethyl carbonochloridate following the procedure described in the synthesis of compound 9.7. The product was purified by flash column chromatography on silica gel (Combiflash®, 3.5% methanol in DCM). MS(ES): 568.3 [M+H]⁺.

Synthesis of I-129. Compound I-129 was prepared from compound 129.6 following the procedure described in the synthesis of compound I-9. The product was purified by flash column chromatography on silica gel (Combiflash®, 3.2% methanol in DCM). MS(ES) 531.3 [M+H]⁺, ¹H-NMR (400 MHz, DMSO-d₆): δ 8.81 (s, 1H), 8.48 (s, 1H), 8.41 (s, 1H), 7.89 (s, 1H), 7.38-7.32 (m, 2H), 7.13-7.09 (m, 2H), 4.27-4.24 (m, 4H), 3.77 (s, 3H), 3.38 (s, 3H), 3.26-3.22 (m, 2H), 2.06-1.98 (m, 2H), 1.70-1.67 (m, 1H), 1.46-1.43 (m, 1H), 1.31-1.29 (m, 1H), 0.89-0.86 (m, 1H).

### Example I-130: 3-((1S,3S)-3-(9-(benzo[d][1,3]dioxol-5-yl)-3-methyl-2-oxo-8-(thiazol-5-yl)-2,3-dihydro-1H-imidazo[4,5-d]pyrrolo[1,2-b]pyridazin-1-yl)cyclopentyl)oxazolidin-2-one

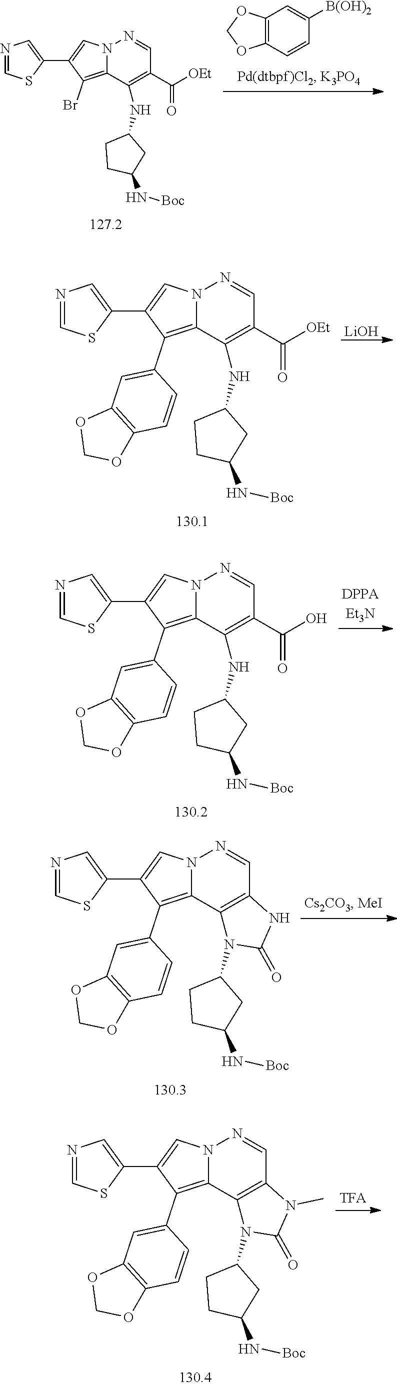

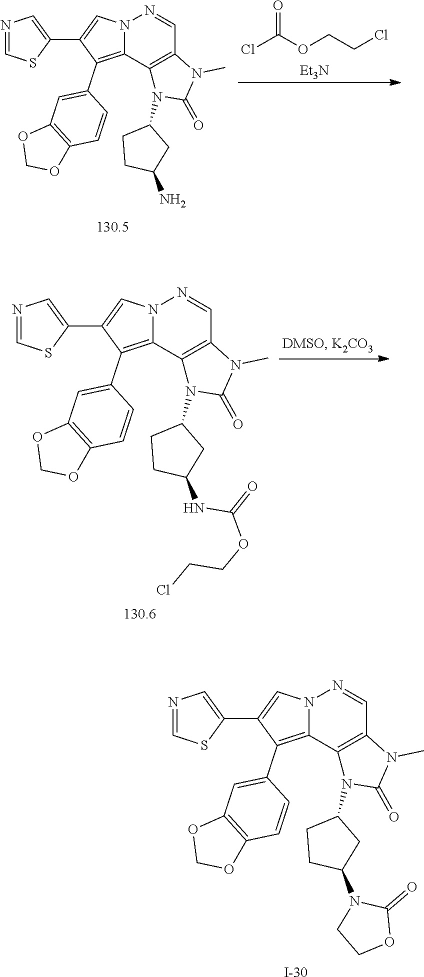

Synthesis of compound 130.1. Compound 130.1 was prepared from compound 127.2 and benzo[d][1,3]dioxol-5-ylboronic acid following the procedure described in the synthesis of compound 59.12. The product was purified by flash column chromatography on silica gel (60% ethyl acetate in n-hexane), MS (ES): m/z 592.6 [M+H]⁺.

Synthesis of compound 130.2. Compound 130.2 was prepared from compound 130.1 following the procedure described in the synthesis of compound 1.10. The product was directly used in the next step without purification. MS (ES): m/z 564.5 [M+H]⁺.

Synthesis of compound 130.3. Compound 130.3 was prepared from compound 130.2 following the procedure described in the synthesis of compound 1.11. The product was purified by flash column chromatography on silica gel (2.2% methanol in DCM). MS (ES): m/z 561.5 [M+H]⁺.

Synthesis of compound 130.4. Compound 130.4 was prepared from compound 130.3 following the procedure described in the synthesis of compound 1.12. The product was purified by flash column chromatography on silica gel (2.0% methanol in DCM). MS (ES): m/z 575.6 [M+H]⁺.

Synthesis of compound 130.5. Compound 130.5 was prepared from compound 130.4 following the procedure described in the synthesis of compound 1.14. The product was used in the next step without purification. MS (ES): m/z 475.4 [M+H]⁺.

Synthesis of compound 130.6. Compound 130.6 was prepared from compound 130.5 and 2-chloroethyl carbonochloridate following the procedure described in the synthesis of compound 9.7. The product was purified by flash column chromatography on silica gel (Combiflash®, 3.0% methanol in DCM). MS(ES): m/z 582.8 [M+H]⁺.

Synthesis of I-130. Compound I-130 was prepared from compound 130.6 following the procedure described in the synthesis of compound I-9. The product was purified by flash column chromatography on silica gel (Combiflash®, 3.5% methanol in DCM). MS(ES): m/z 545.3 [M+H]⁺, ¹H-NMR (400 MHz, DMSO-d₆): δ 8.86 (s, 1H), 8.50 (s, 1H), 8.42 (s, 1H), 7.95 (d, J=8.8 Hz, 1H), 7.16-7.08 (m, 1H), 7.02 (d, J=12.8 Hz, 1H), 6.93 (t, J=6.4 Hz, 1H), 6.17-6.11 (m, 2H), 4.30-4.26 (m, 3H), 3.95 (s, 1H), 3.81 (s, 1H), 3.38 (s, 3H), 2.54 (s, 2H), 2.09-2.02 (m, 2H), 1.77 (m, 1H), 1.53-1.31 (m, 1H), 1.10-0.85 (m, 1H).

### Example I-131: methyl ((1R,3R)-3-(8-(3,6-dihydro-2H-pyran-4-yl)-3-methyl-2-oxo-9-(6-oxo-5-(trifluoromethyl)-1,6-dihydropyridazin-3-yl)-2,3-dihydro-1H-imidazo[4,5-d]pyrrolo[1,2-b]pyridazin-1-yl)cyclopentyl)carbamate

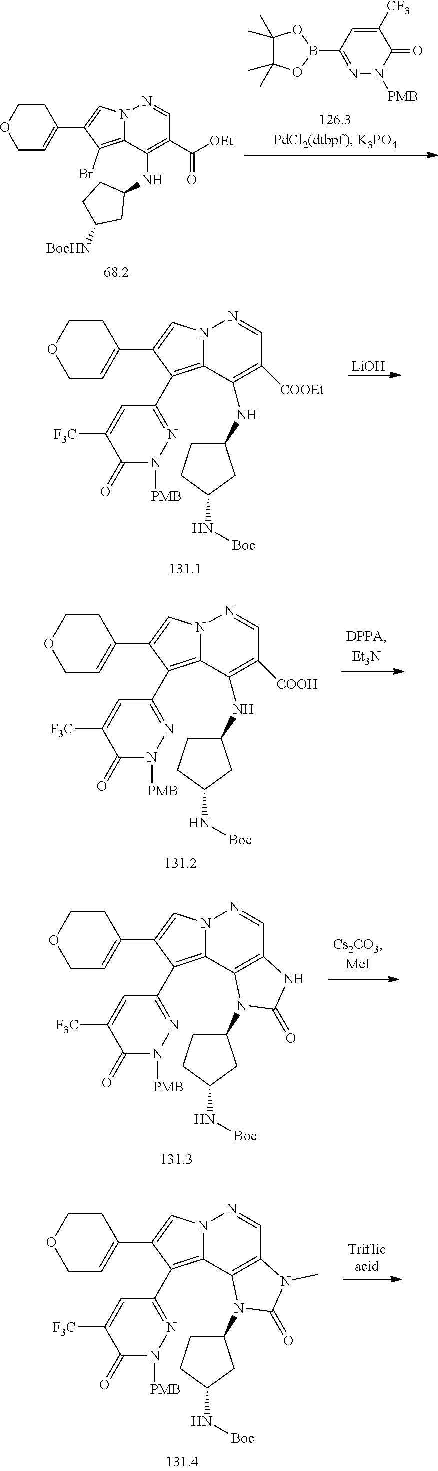

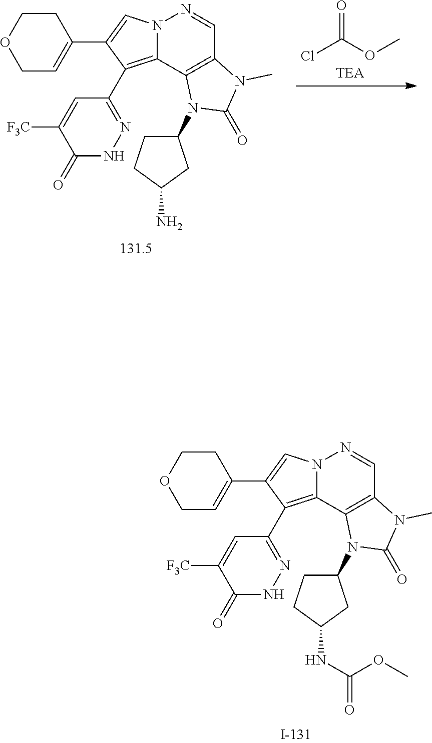

Synthesis of compound 131.1. Compound 131.1 was prepared from compound 68.2 and 126.3 following the procedure described in the synthesis of compound 59.12. The product was purified by flash column chromatography on silica gel (2.6% methanol in DCM). MS (ES): m/z 753.79 [M+H]⁺.

Synthesis of compound 131.2. Compound 131.2 was prepared from compound 131.1 following the procedure described in the synthesis of compound 1.10. The product was triturated with n-pentane, dried under vacuum and used in the next step without purification. MS (ES): m/z 725.7 [M+H]⁺.

Synthesis of compound 131.3. Compound 131.3 was prepared from compound 131.2 following the procedure described in the synthesis of compound 1.11. The product was purified by combi-flash flash column chromatography on silica gel (2.4% methanol in DCM). MS (ES): m/z 722.7 [M+H]⁺.

Synthesis of compound 131.4. Compound 131.4 was prepared from compound 131.3 following the procedure described in the synthesis of compound 1.12. The product was purified by flash column chromatography on silica gel (2.7% methanol in DCM). MS (ES): m/z 736.7 [M+H]⁺.

Synthesis of compound 131.5. Compound 131.5 was prepared from compound 131.4 following the procedure described in the synthesis of compound 1.14. The product was triturated with n-pentane and dried under reduced pressure. It was used in the next step without purification. MS (ES): m/z 516.5 [M+H]⁺.

Synthesis of compound I-131. Compound I-131 was prepared from compound 131.5 following the procedure described in the synthesis of compound I-1. The product was purified by flash column chromatography on silica gel (Combiflash®, 3.6% methanol in DCM). MS (ES): m/z 574.3 [M+H]⁺, ¹H NMR (400 MHz, DMSO-d₆): δ 13.96 (s, 1H), 8.53 (s, 1H), 8.03 (s, 1H), 8.00 (s, 1H), 7.06 (m, 1H), 5.46 (m, 1H), 4.09 (m, 2H), 3.98 (m, 3H), 3.73 (m, 3H), 3.46 (m, 3H), 3.39 (s, 3H), 2.31 (s, 1H), 2.10 (m, 1H), 2.00 (m, 1H), 1.75 (s, 1H), 1.45 (m, 1H), 1.06 (s, 1H).

### Example I-132: methyl ((1R,3R)-3-(3-methyl-8-(1-methyl-1H-pyrazol-4-yl)-2-oxo-9-(6-oxo-5-(trifluoromethyl)-1,6-dihydropyridazin-3-yl)-2,3-dihydro-1H-imidazo[4,5-d]pyrrolo[1,2-b]pyridazin-1-yl)cyclopentyl)carbamate

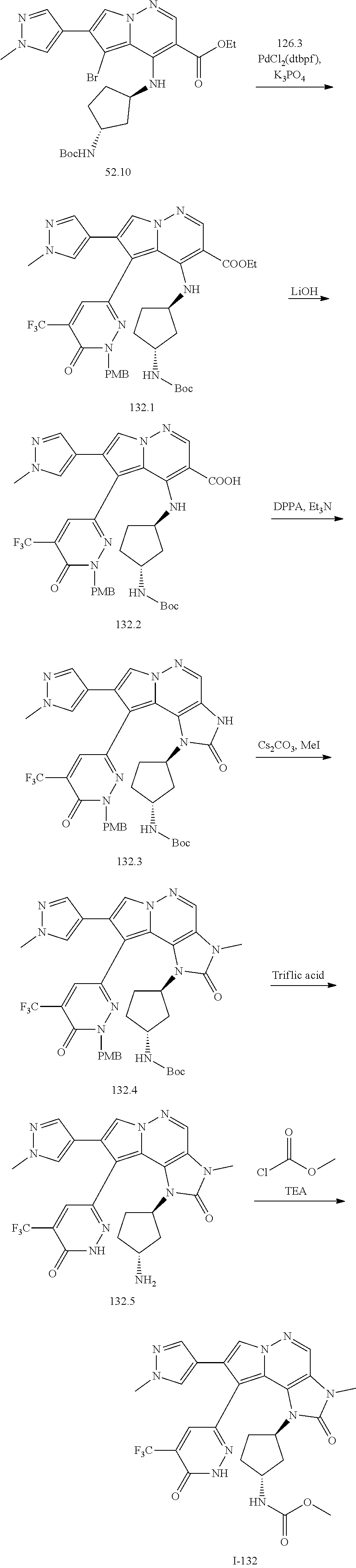

Synthesis of compound 132.1. Compound 132.1 was prepared from compound 52.10 and 126.3 following the procedure described in the synthesis of compound 59.12. The product was purified by flash column chromatography on silica gel (2.7% methanol in DCM). MS (ES): m/z 751.9 [M+H]⁺.

Synthesis of compound 132.2. Compound 132.2 was prepared from compound 132.1 following the procedure described in the synthesis of compound 1.10. The product was triturated with n-pentane, dried under vacuum and used in the next step without purification. MS (ES): m/z 723.8 [M+H]⁺.

Synthesis of compound 132.3. Compound 132.3 was prepared from compound 132.2 following the procedure described in the synthesis of compound 1.11. The product was purified by combi-flash flash column chromatography on silica gel (2.4% methanol in DCM). MS (ES): m/z 720.9 [M+H]⁺.

Synthesis of compound 132.4. Compound 132.4 was prepared from compound 132.3 following the procedure described in the synthesis of compound 1.12. The product was purified by flash column chromatography on silica gel (2.6% methanol in DCM). MS (ES): m/z 734.7 [M+H]⁺.

Synthesis of compound 132.5. Compound 132.5 was prepared from compound 132.4 following the procedure described in the synthesis of compound 1.14. The product was triturated with n-pentane and dried under reduced pressure. It was used in the next step without purification. MS (ES): m/z 514.5 [M+H]⁺.

Synthesis of compound I-132. Compound I-132 was prepared from compound 132.5 following the procedure described in the synthesis of compound 11-1. The product was purified by flash column chromatography on silica gel (Combiflash®, 3.5% methanol in DCM). MS (ES): m/z 572.5 [M+H]⁺. ¹H NMR (400 MHz, DMSO-d₆): δ 13.97 (s, 1H), 8.53 (d, J=2.4 Hz, 1H), 8.14 (d, J=2.4 Hz, 1H), 7.95 (s, 1H), 7.62 (s, 1H), 7.38 (s, 1H), 7.08 (s, 1H), 4.11 (t, J=9.2 Hz, 2H), 3.81 (d, J=2.8 Hz, 3H), 3.65 (d, J=22.4 Hz, 3H), 3.48 (s, 3H), 2.33 (s, 1H), 2.09 (d, J=8.8 Hz, 1H), 2.00 (d, J=4.4 Hz, 2H), 1.72 (s, 2H).

### Example I-133: 3-((1S,3S)-3-(3-methyl-9-(1-methyl-1H-indazol-5-yl)-2-oxo-8-(thiazol-5-yl)-2,3-dihydro-1H-imidazo[4,5-d]pyrrolo[1,2-b]pyridazin-1-yl)cyclopentyl)oxazolidin-2-one

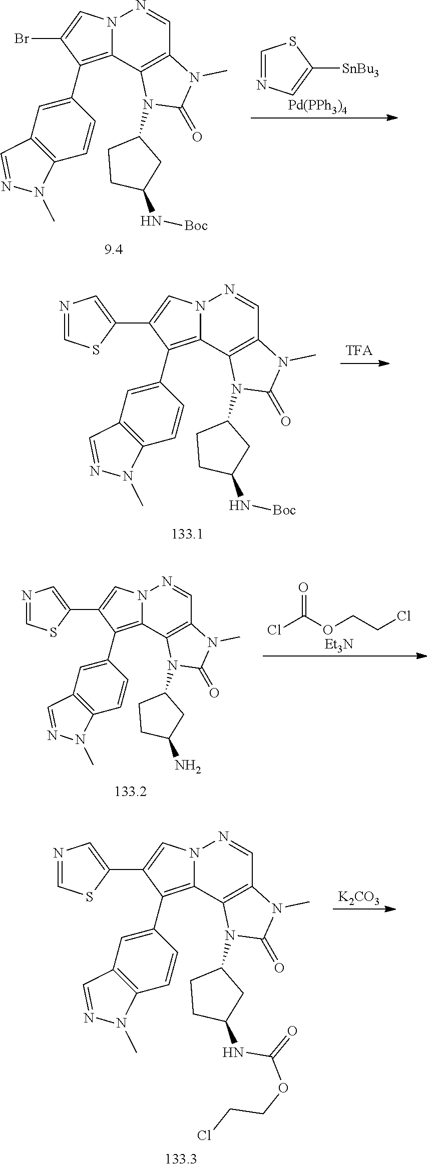

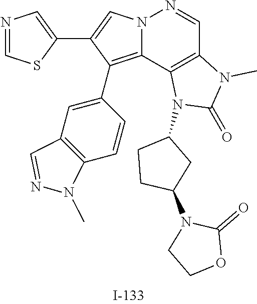 [SMILES: Cn1ncc2cc(-c3c(-c4cncs4)cn4ncc5c(c34)n([C@H]3CC[C@H](N4CCOC4=O)C3)c(=O)n5C)ccc21]

Synthesis of compound 133.1. Compound 133.1 was prepared from compound 9.4 and 5-(tributylstannyl)thiazole following the procedure described in the synthesis of compound I-116. The product was purified by flash column chromatography on silica gel (40% ethyl acetate in hexane). MS(ES): m/z 585.6 [M+H]⁺.

Synthesis of compound 133.2. Compound 133.2 was prepared from compound 133.1 following the procedure described in the synthesis of compound 1.14. The product was used in the next step without purification. MS(ES): m/z 485.6 [M+H]⁺.

Synthesis of compound 133.3. Compound 133.3 was prepared from compound 133.2 and 2-chloroethyl carbonochloridate following the procedure described in the synthesis of compound 9.7. The product was purified by flash column chromatography on silica gel (Combiflash®, 2.6% methanol in DCM). MS(ES): m/z 591.7 [M+H]⁺.

Synthesis of I-133. Compound I-133 was prepared from compound 133.3 following the procedure described in the synthesis of compound I-9. The product was purified by flash column chromatography on silica gel (Combiflash®, 2.3% methanol in DCM). MS(ES): m/z 555.6 [M+H]⁺, ¹H-NMR (400 MHz, DMSO-d₆): δ 8.75 (s, 1H), 8.49 (d, J=16 Hz, 2H), 8.14 (d, J=16 Hz, 1H), 7.88 (m, 3H), 7.44 (dd, J=28 Hz, 1H), 4.28 (m, 1H), 4.22 (s, 3H), 4.07 (m, 1H), 3.34 (s, 3H), 3.30-2.86 (m, 1H), 2.62 (m, 1H), 2.33-1.64 (m, 1H), 2.22 (m, 2H), 1.40 (m, 2H), 1.37 (m, 1H), 0.87 (m, 1H), 0.49-0.01 (m, 1H).

### Example I-134: N-(((1r,3r)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclobutyl)methyl)acetamide

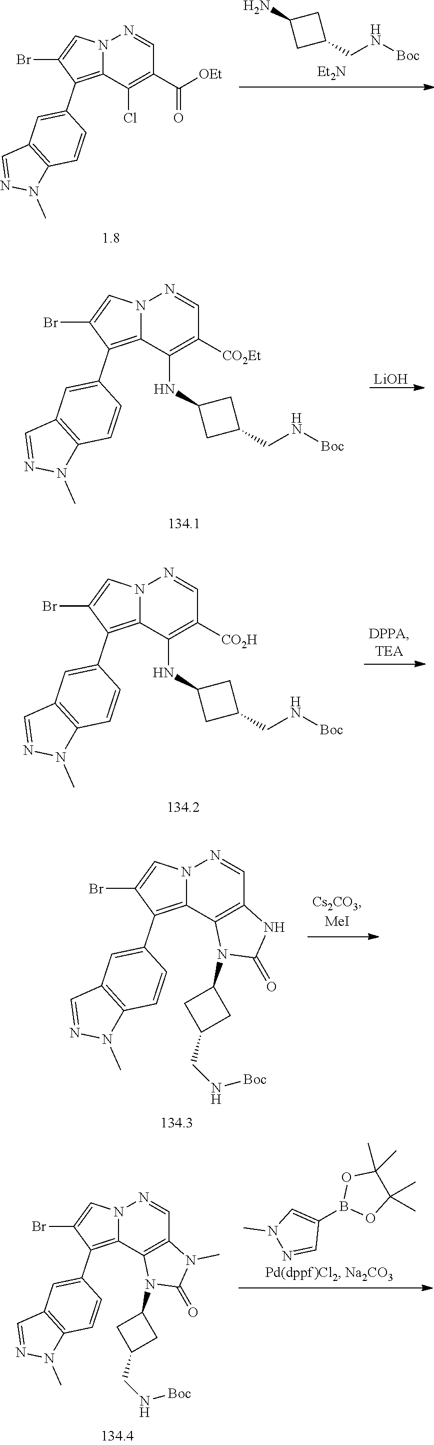

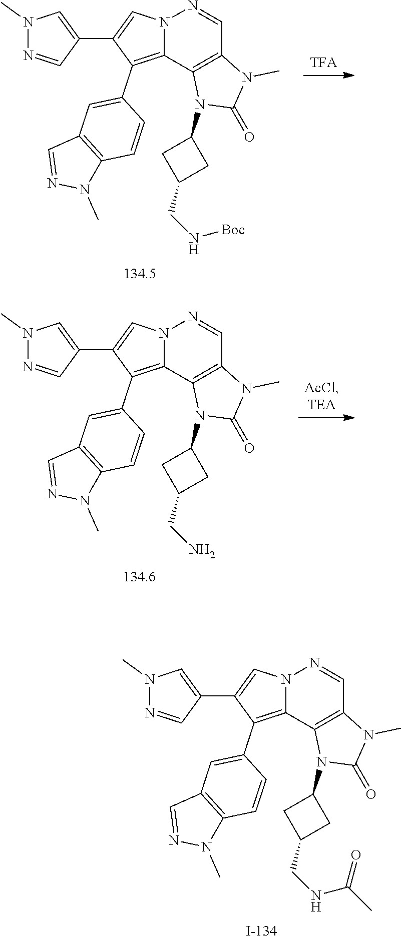

Synthesis of compound 134.1. Compound 134.1 was prepared from compound 1.8 and tert-butyl (((1r,3r)-3-aminocyclobutyl)methyl)carbamate following the procedure described in the synthesis of compound 1.9. The product was purified by flash column chromatography on silica gel (Combiflash®, 50% ethyl acetate in hexane). MS(ES): m/z 597.4 and 599.3 [M+H]⁺.

Synthesis of compound 134.2. Compound 134.2 was prepared from compound 134.1 following the procedure described in the synthesis of compound 1.10. The product was triturated with pentane, dried under vacuum and used in the next step without purification. MS(ES): m/z 569.30 and 571.30 [M+H]⁺.

Synthesis of compound 134.3. Compound 134.3 was prepared from compound 134.2 following the procedure described in the synthesis of compound 1.11. The product was purified by flash column chromatography on silica gel (Combiflash®, 1.8% methanol in DCM). MS(ES): m/z 566.30 and 568.30 [M+H]⁺.

Synthesis of compound 134.4. Compound 134.4 was prepared from compound 134.3 following the procedure described in the synthesis of compound 1.12. The product was purified by flash column chromatography on silica gel (1.4% methanol in DCM). MS(ES): m/z 580.3 and 582.3 [M+H]⁺.

Synthesis of compound 134.5. Compound 134.5 was prepared from compound 134.4 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (Combiflash®, 3.1% methanol in DCM). MS(ES): m/z 582.31 [M+H]⁺.

Synthesis of compound 134.6. Compound 134.6 was prepared from compound 134.5 following the procedure described in the synthesis of compound 1.14. The product was triturated with pentane and dried under reduced pressure. It was used in the next step without purification. MS(ES): 482.28 [M+H]⁺.

Synthesis of compound I-134. To a solution of 134.6 (0.060 g, 0.124 mmol, 1.0 equiv) and triethylamine (0.052 mL, 0.375 mmol, 3.0 equiv) in THF (2.0 mL) at 0° C. was added acetyl chloride (0.020 g, 0.25 mmol, 2.0 equiv) and stirred at room temperature for 1 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 3% methanol in DCM) to afford I-134 (0.020 g, 31%). MS(ES): 524.24 [M+H]⁺. ¹H-NMR (400 MHz, DMSO-d₆): δ 8.40 (d, J=2.0 Hz, 1H), 8.14 (s, 1H), 8.06 (s, 1H), 7.82 (d, J=8.8 Hz, 1H), 7.73 (s, 1H), 7.40-7.34 (m, 2H), 7.25 (s, 1H), 6.96 (s, 1H), 4.14 (s, 3H), 3.82-3.78 (m, 1H), 3.67 (s, 3H), 3.36 (s, 3H), 2.88-2.83 (m, 1H), 2.75-2.67 (m, 1H), 1.90 (s, 3H), 1.72 (s, 3H), 1.33-1.30 (m, 1H), 0.96 (m, 1H).

### Example I-135: 2 methyl ((1R,3R)-3-(9-(3,3-dimethyl-2-oxoindolin-6-yl)-3-methyl-2-oxo-8-(thiazol-2-yl)-2,3-dihydro-1H-imidazo[4,5-d]pyrrolo[1,2-b]pyridazin-1-yl)cyclopentyl)carbamate

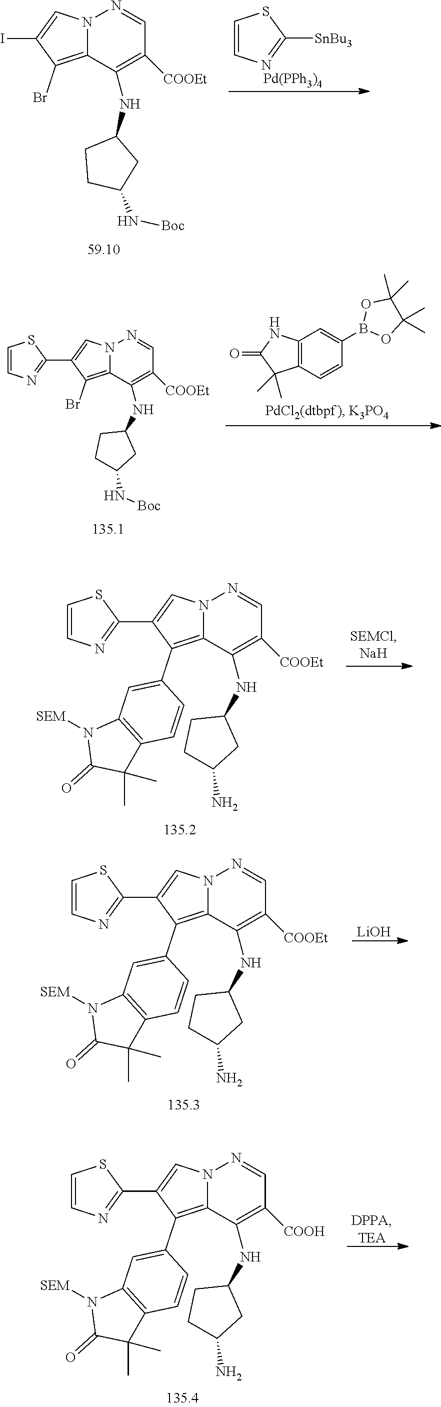

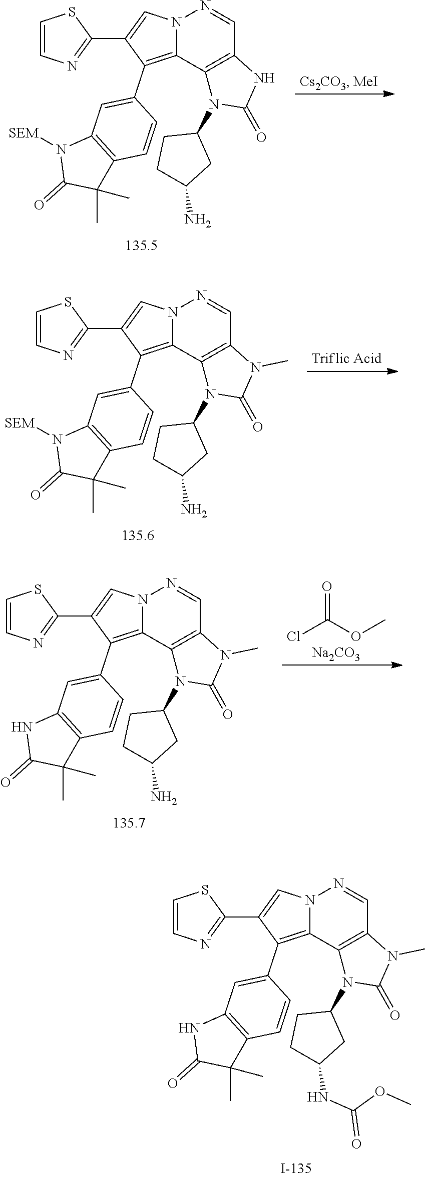

Synthesis of compound 135.1. Compound 135.1 was prepared from compound 59.10 and 2-(tributylstannyl)thiazole following the procedure described in the synthesis of compound I-116. The product was purified by flash column chromatography on silica gel (Combiflash®, 18% ethyl acetate in hexane). MS(ES): m/z 550.4 and 552.1 [M+H]⁺.

Synthesis of compound 135.2. Compound 135.2 was prepared from compound 135.1 and 3,3-dimethyl-6-(4,4,5,5-tetramethyl-1,3,2-dioxaborolan-2-yl)indolin-2-one following the procedure described in the synthesis of compound 59.12. The product was purified by flash column chromatography on silica gel (Combiflash®, 35% ethyl acetate in hexane). MS(ES): m/z 661.9 [M+H]⁺.

Synthesis of compound 135.3. To a solution of 135.2 (0.275 g, 0.436 mmol, 1.0 equiv) in THF (6 mL) was added sodium hydride (60% dispersion in mineral oil, 0.035 g, 0.873 mmol, 2.0 equiv) in small portions at 0° C. and stirred for 30 min. To the mixture was added 2-(trimethylsilyl)ethoxymethyl chloride (0.078 g, 0.47 mmol, 1.1 equiv) at 0° C. and the reaction mixture was stirred at room temperature for 1 h. It was transferred into water and extracted with ethyl acetate. The combined organic layers were washed with brine, dried over anhydrous sodium sulfate, filtered and concentrated under reduced pressure. The residue was purified by flash column chromatography on silica gel (Combiflash®, 22% ethyl acetate in hexane) to afford 135.3 (0.170 g, 51%). MS(ES): m/z 761.2 [M+H]⁺.

Synthesis of compound 135.4. Compound 135.4 was prepared from compound 135.3 following the procedure described in the synthesis of compound 1.10. The product was triturated with pentane, dried under vacuum and used in the next step without purification. MS(ES): m/z 733.2 [M+H]⁺.

Synthesis of compound 135.5. Compound 135.5 was prepared from compound 135.4 following the procedure described in the synthesis of compound 1.11. The product was purified by flash column chromatography on silica gel (Combiflash®, 0-2.6% methanol in DCM). MS(ES): m/z 730.6 [M+H]⁺.

Synthesis of compound 135.6 Compound 135.6 was prepared from compound 135.5 following the procedure described in the synthesis of compound 1.12. The product was purified by flash column chromatography on silica gel (0-1.9% methanol in DCM). MS(ES): m/z 644.3 [M+H]⁺.

Synthesis of compound 135.7. Compound 135.7 was prepared from compound 135.6 following the procedure described in the synthesis of compound 21.8. The product was used in the next step without purification. MS(ES): m/z 514.2 [M+H]⁺.

Synthesis of compound I-135. Compound I-135 was prepared from compound 135.7 following the procedure described in the synthesis of compound I-1. The product was purified by flash column chromatography on silica gel (0-4.1% methanol in DCM). MS(ES): m/z 572.2 [M+H]⁺. ¹H-NMR (400 MHz, DMSO-d₆): δ 10.10 (s, 1H), 8.81 (s, 1H), 8.42 (s, 1H), 8.29 (s, 1H), 7.78 (s, 1H), 7.38 (d, J=6.4 Hz, 1H), 7.02 (s, 1H), 6.84 (s, 1H), 6.48 (m, 1H), 3.50 (s, 4H), 3.38 (s, 4H), 2.13 (m, 2H), 1.98-1.90 (m, 3H), 1.36 (s, 6H), 1.26-1.16 (m, 1H).

### Example I-136: 1-((1s,4s)-4-cyclopropyl-4-hydroxycyclohexyl)-3-methyl-9-(1-methyl-1H-indazol-5-yl)-8-(1-methyl-1H-pyrazol-4-yl)-1,3-dihydro-2H-imidazo[4,5-d]pyrrolo[1,2-b]pyridazin-2-one

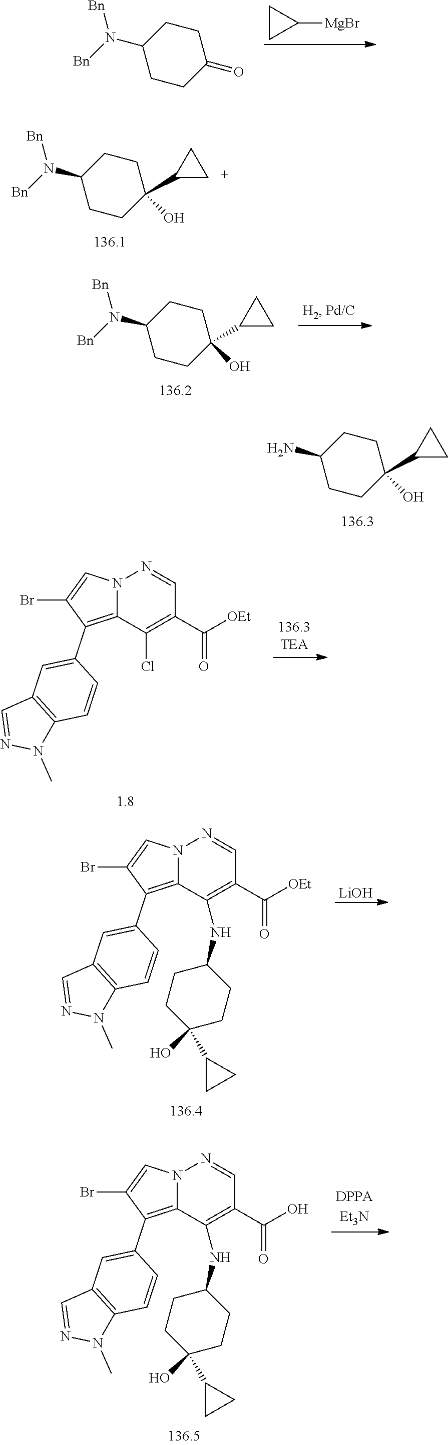

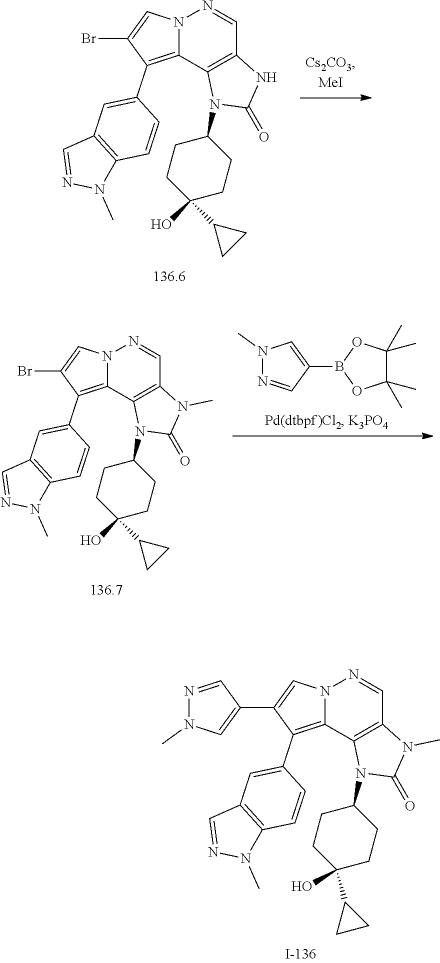

Synthesis of compound 136.1 and 136.2. To a solution of 4-(dibenzylamino)cyclohexan-1-one (10.0 g, 3.412 mmol, 1.0 equiv) in THF (15 mL) was added cyclopropylmagnesium bromide (0.16 M in THF, 319 mL, 5.119 mmol, 1.5 equiv) at 0° C. The reaction mixture was stirred at room temperature for 16 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 10% ethyl acetate in hexane) to afford mixture of diastereomers of 136.1 and 136.2 (2.0 g, 17.5%). MS(ES): m/z 336.4 [M+H]⁺. The mixture of was spread by SFC: (column: CHIRALPAK IG (250*30 mm), 5 μm; mobile phase: (A) CO₂ (B) 0.1% NH₃ in IPA: MeCN (50: 50); flow rate: 100 mL/min) to afford 136.1 (1.0 g, 8.8%): MS(ES): m/z 336.4 [M+H]⁺, and 136.2 (0.10 g, 8.8%): MS(ES): m/z 336.4 [M+H]⁺.

Synthesis of compound 136.3. A mixture of 136.1 (1.5 g, 2.985 mmol, 1.0 equiv) and 10% Pd/C (w/w) in methanol (15 mL) was stirred under H₂ (1 atm) for 5 h. It was diluted with methanol and filtered through a pad of Celite®, rinsed with 10% methanol in DCM, filtered and concentrated under reduced pressure. The residue was used in the next step to afford 136.3 (0.600 g, 86%). MS(ES): m/z 156.4 [M+H]⁺.

Synthesis of compound 136.4. Compound 136.4 was prepared from compound 136.3 and 1.8 following the procedure described in the synthesis of compound 1.9. The product was purified by flash column chromatography on silica gel (Combiflash®, 50% ethyl acetate in hexane). MS(ES): m/z 552.4 and 554.4 [M+H]⁺.

Synthesis of compound 136.5. Compound 136.5 was prepared from compound 136.4 following the procedure described in the synthesis of compound 1.10. The product was triturated with pentane, dried under vacuum and used in the next step without purification. MS(ES): m/z 524.2 and 526.2 [M+H]⁺.

Synthesis of compound 136.6. Compound 136.6 was prepared from compound 136.5 following the procedure described in the synthesis of compound 1.11. The product was purified by flash column chromatography on silica gel (Combiflash®, 1.8% methanol in DCM). MS(ES): m/z 521.3 & 523.3 [M+H]⁺.

Synthesis of compound 136.7. Compound 136.7 was prepared from compound 136.6 following the procedure described in the synthesis of compound 1.12. The product was purified by flash column chromatography on silica gel (1.4% methanol in DCM). MS(ES): m/z 535.1 and 537.1 [M+H]⁺.

Synthesis of compound I-136. Compound I-136 was prepared from compound 136.7 and 1-methyl-4-(4,4,5,5-tetramethyl-1,3,2-dioxaborolan-2-yl)-1H-pyrazole following the procedure described in the synthesis of compound 59.12. The product was purified by flash column chromatography on silica gel (Combiflash®, 0-3.2% methanol in DCM). MS(ES): m/z 537.3 [M+H]⁺, ¹H-NMR (400 MHz, DMSO-d₆): δ 8.38 (d, J=1.6 Hz, 1H), 8.13-8.09 (m, 2H), 7.81 (d, J=8.0 Hz, 1H), 7.75 (s, 1H), 7.38 (d, J=8.4 Hz, 1H), 7.20 (s, 1H), 6.84 (s, 1H), 4.09 (s, 3H), 3.64 (s, 3H), 3.28 (s, 3H), 3.22 (m, 1H), 1.14 (m, 1H), 1.00 (m, 1H), 0.97 (m, 2H), 0.15 (t, J=10.4 Hz, 1H), 0.08-0.00 (m, 4H), −0.44-−0.39 (m, 1H).

### Example I-137: 3-methyl-9-(1-methyl-1H-indazol-5-yl)-8-(1-methyl-1H-pyrazol-4-yl)-1-((1r,4r)-4-methyl-4-(2-oxopyrrolidin-1-yl)cyclohexyl)-1,3-dihydro-2H-imidazo[4,5-d]pyrrolo[1,2-b]pyridazin-2-one

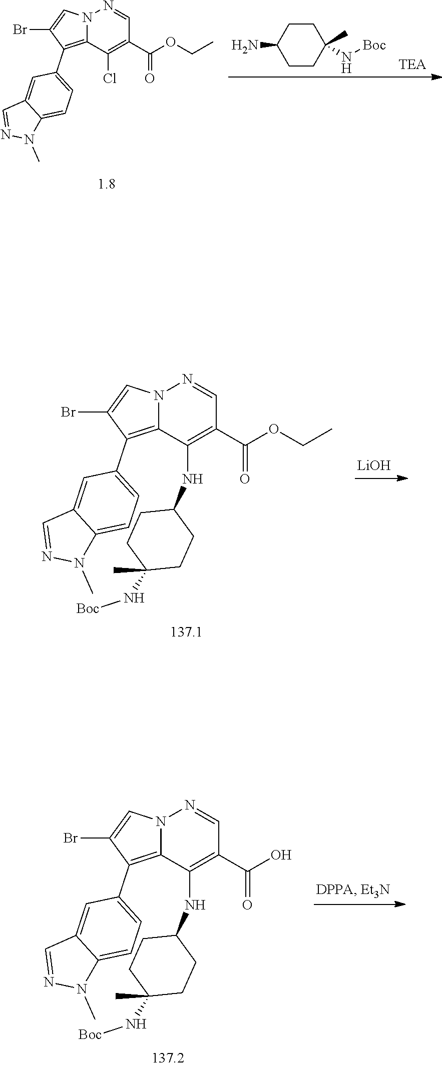

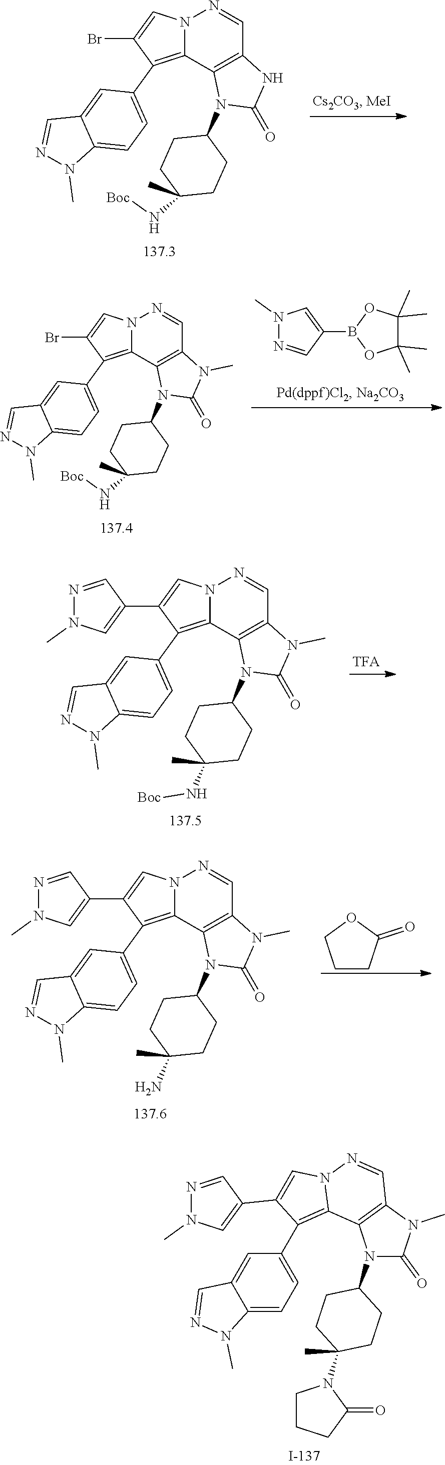

Synthesis of compound 137.1. Compound 137.1 was prepared from compound 1.8 and tert-butyl ((1r,4r)-4-amino-1-methylcyclohexyl)carbamate following the procedure described in the synthesis of compound 1.9. The product was purified by flash column chromatography on silica gel (Combiflash®, 53% ethyl acetate in hexane). MS(ES): m/z 625.2 and 627.1 [M+H]⁺.

Synthesis of compound 137.2. Compound 137.2 was prepared from compound 137.1 following the procedure described in the synthesis of compound 1.10. The product was triturated with pentane, dried under vacuum and used in the next step without purification. MS(ES): m/z 597.4 and 599.4 [M+H]⁺.

Synthesis of compound 137.3. Compound 137.3 was prepared from compound 137.2 following the procedure described in the synthesis of compound 1.11. The product was purified by flash column chromatography on silica gel (Combiflash®, 1.8% methanol in DCM). MS(ES): m/z 594.4 and 596.2 [M+H]⁺.

Synthesis of compound 137.4. Compound 137.4 was prepared from compound 137.3 following the procedure described in the synthesis of compound 1.12. The product was purified by flash column chromatography on silica gel (1.2% methanol in DCM). MS(ES): m/z 608.1 and 610.4 [M+H]⁺.

Synthesis of compound 137.5. Compound 137.5 was prepared from compound 137.4 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (Combiflash®, 0-2.8% methanol in DCM). MS(ES): m/z 610.2

Synthesis of compound 137.6. Compound 137.6 was prepared from compound 137.5 following the procedure described in the synthesis of compound 1.14. The product used in next step without purification. MS(ES): m/z 510.1 [M+H]⁺.

Synthesis of I-137. A mixture of 137.6 (0.140 g, 0.275 mmol, 1.0 equiv) and dihydrofuran-2(3H)-one (7 mL) was stirred at 220° C. for 1 h in a microwave reactor. It was quenched in water and extracted with ethyl acetate. The combined organic layers were washed with brine, dried over anhydrous sodium sulfate, filtered and concentrated under reduced pressure. The residue was purified by flash column chromatography on silica gel (Combiflash®, 4.1% methanol in DCM) to afford I-137 (0.018 g, 11%). MS(ES): m/z 578.4 [M+H]⁺. ¹H NMR (DMSO-d₆, 400 MHz): δ 8.40 (s, 1H), 8.15 (s, 1H), 8.12 (d, J=0.8 Hz, 1H), 7.84-7.80 (m, 2H), 7.40-7.38 (dd, J=8.4 Hz & 1.6 Hz, 1H), 7.21 (s, 1H), 6.88 (d, J=0.4 Hz, 1H), 4.12 (s, 3H), 3.65 (s, 3H), 3.36 (s, 3H), 3.15-3.13 (m, 1H), 2.89-2.82 (m, 2H), 2.34-2.24 (m, 2H), 2.21-2.13 (m, 2H), 2.11-2.02 (m, 1H), 1.84-1.79 (m, 2H), 1.56-1.53 (m, 1H), 1.24-1.22 (m, 2H), 1.19 (s, 3H), 1.16-1.11 (m, 1H), 0.88-0.85 (m, 1H).

### Example I-138: 3-((1S,3S)-3-(9-(benzo[d][1,3]dioxol-5-yl)-8-(3-methoxy-1-methyl-1H-pyrazol-4-yl)-3-methyl-2-oxo-2,3-dihydro-1H-imidazo[4,5-d]pyrrolo[1,2-b]pyridazin-1-yl)cyclopentyl)oxazolidin-2-one

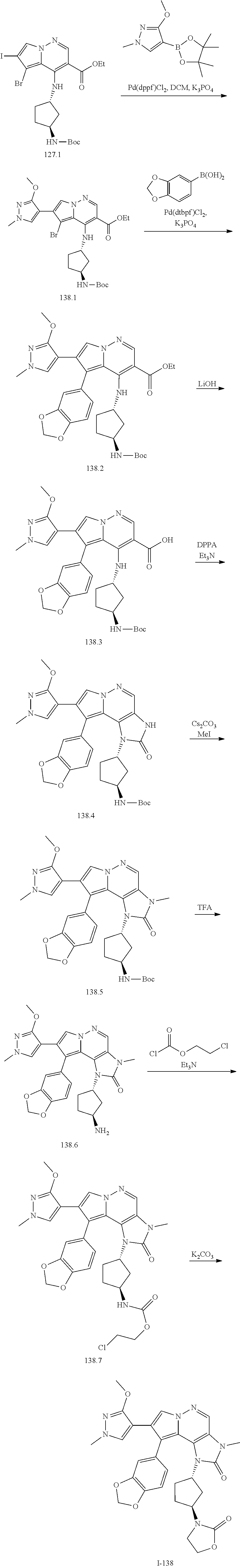

Synthesis of compound 138.1. Compound 138.1 was prepared from compound 127.1 and 3-methoxy-1-methyl-4-(4,4,5,5-tetramethyl-1,3,2-dioxaborolan-2-yl)-1H-pyrazole following the procedure described in the synthesis of compound 54.3. The product was purified by flash column chromatography on silica gel (50% ethyl acetate in n-hexane). MS(ES): m/z 577.5 and 579.6 [M+H]⁺.

Synthesis of compound 138.2. Compound 138.2 was prepared from compound 138.1 and benzo[d][1,3]dioxol-5-ylboronic acid following the procedure described in the synthesis of compound 59.12. The product was purified by flash column chromatography on silica gel (68% ethyl acetate in hexane), MS(ES): m/z 619.4 [M+H]⁺.

Synthesis of compound 138.3. Compound 138.3 was prepared from compound 138.2 following the procedure described in the synthesis of compound 1.10. The product was triturated with pentane, dried under vacuum and used in the next step without purification. MS(ES): m/z 591.3 [M+H]⁺.

Synthesis of compound 138.4. Compound 138.4 was prepared from compound 138.3 following the procedure described in the synthesis of compound 1.11. The product was purified by flash column chromatography on silica gel (Combiflash®, 1.8% methanol in DCM). MS(ES): m/z 588.5 [M+H]⁺.

Synthesis of compound 138.5. Compound 138.5 was prepared from compound 138.4 following the procedure described in the synthesis of compound 1.12. The product was purified by flash column chromatography on silica gel (1.4% methanol in DCM). MS(ES): m/z 602.6 [M+H]⁺.

Synthesis of compound 138.6. Compound 138.6 was prepared from compound 138.5 following the procedure described in the synthesis of compound 1.14. The product was used in the next step without purification. MS(ES): m/z 502.6 [M+H]⁺.

Synthesis of compound 138.7. Compound 138.7 was prepared from compound 138.6 and 2-chloroethyl carbonochloridate following the procedure described in the synthesis of compound 9.7. The product was purified by flash column chromatography on silica gel (Combiflash®, 3.2% methanol in DCM). MS(ES): m/z 609.1 [M+H]⁺.

Synthesis of I-138. Compound I-138 was prepared from compound 138.7 following the procedure described in the synthesis of compound I-9. The product was purified by flash column chromatography on silica gel (Combiflash®, 4.1% methanol in DCM). MS(ES) m/z: 572.4 [M+H]⁺. ¹H-NMR (400 MHz, DMSO-d₆): δ 8.37 (s, 1H), 8.01 (s, 1H), 7.11 (d, J=7.6 Hz, 1H), 6.96 (d, J=9.2 Hz, 1H), 6.86 (d, J=7.2 Hz, 1H), 6.48 (m, 1H), 6.13 (m, 2H), 4.34-4.27 (m, 3H), 4.06 (s, 3H), 3.73-3.68 (m, 1H), 3.55 (s, 3H), 3.36 (s, 3H), 2.52 (m, 1H), 2.03 (m, 2H), 1.76 (m, 1H), 1.46 (m, 1H), 1.34 (m, 1H), 1.17 (m, 1H), 1.03 (m, 1H).

### Example I-139: 3-((1S,3S)-3-(9-(6,7-dihydro-4H-thieno[3,2-c]pyran-2-yl)-3-methyl-2-oxo-8-(thiazol-5-yl)-2,3-dihydro-1H-imidazo[4,5-d]pyrrolo[1,2-b]pyridazin-1-yl)cyclopentyl)oxazolidin-2-one

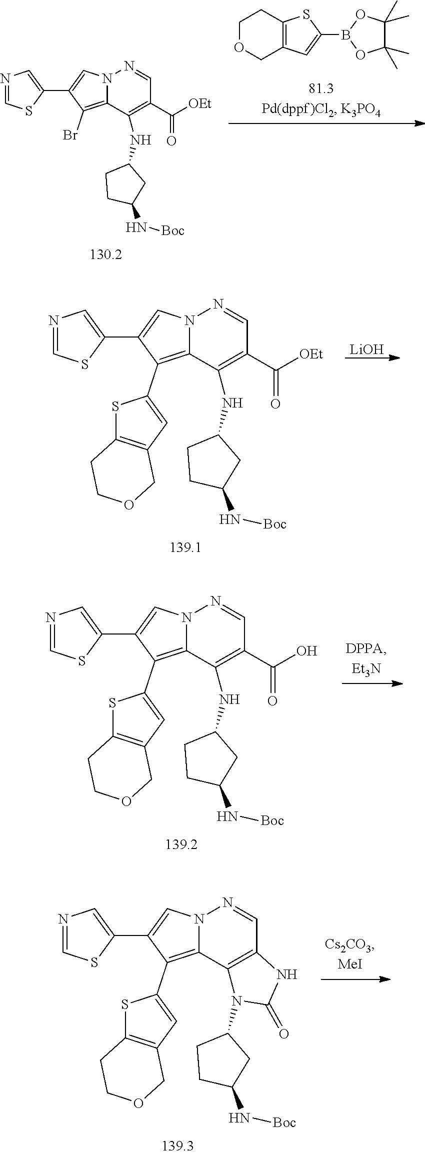

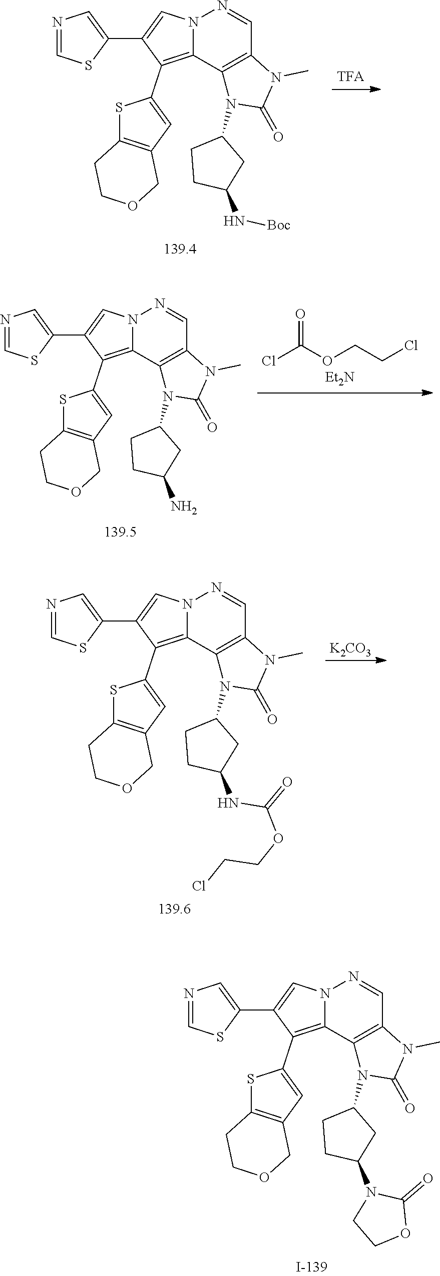

Synthesis of compound 139.1. Compound 139.1 was prepared from compound 130.2 and 81.3 following the procedure described in the synthesis of compound 59.12. The product was purified by flash column chromatography on silica gel (55% ethyl acetate in hexane). MS(ES): m/z 610.70 [M+H]⁺.

Synthesis of compound 139.2. Compound 139.2 was prepared from compound 139.1 following the procedure described in the synthesis of compound 1.10. The product was triturated with pentane, dried under vacuum and used in the next step without purification. MS(ES): m/z 582.68 [M+H]⁺.

Synthesis of compound 139.3. Compound 139.3 was prepared from compound 139.2 following the procedure described in the synthesis of compound 1.11. The product was purified by flash column chromatography on silica gel (Combiflash®, 2.1% methanol in DCM). MS(ES): m/z 579.67 [M+H]⁺.

Synthesis of compound 139.4. Compound 139.4 was prepared from compound 139.3 following the procedure described in the synthesis of compound 1.12. The product was purified by flash column chromatography on silica gel (1.8% methanol in DCM). MS(ES): m/z 593.5 [M+H]⁺.

Synthesis of compound 139.5. Compound 139.5 was prepared from compound 139.4 following the procedure described in the synthesis of compound 1.14. The product was used in the next step without purification. MS(ES): m/z 493.58 [M+H]⁺.

Synthesis of compound 139.6. Compound 139.6 was prepared from compound 139.5 and 2-chloroethyl carbonochloridate following the procedure described in the synthesis of compound 9.7. The product was purified by flash column chromatography on silica gel (Combiflash®, 3.5% methanol in DCM). MS(ES) 599.95 [M+H]⁺.

Synthesis of I-139. Compound I-139 was prepared from compound 139.6 following the procedure described in the synthesis of compound I-9. The product was purified by flash column chromatography on silica gel (Combiflash®, 3.2% methanol in DCM). MS(ES): 563.61 [M+H]⁺. ¹H-NMR (400 MHz, DMSO-d₆): δ 8.96 (s, 1H), 8.57 (s, 1H), 8.47 (s, 1H), 8.05 (s, 1H), 7.01 (d, J=15.2 Hz, 1H), 4.70 (s, 1H), 4.70-4.58 (m, 1H), 4.47-4.43 (m, 1H), 4.33-4.26 (m, 2H), 4.07-3.95 (m, 4H), 3.40 (s, 3H), 2.92 (s, 2H), 2.61 (s, 2H), 2.35 (s, 1H), 1.15 (s, 1H), 1.84 (s, 1H), 1.71-1.64 (m, 1H), 1.60-1.58 (m, 1H).

### Example I-140: 3-((1S,3S)-3-(8-(3,6-dihydro-2H-pyran-4-yl)-9-(6,7-dihydro-4H-thieno[3,2-c]pyran-2-yl)-3-methyl-2-oxo-2,3-dihydro-1H-imidazo[4,5-d]pyrrolo[1,2-b]pyridazin-1-yl)cyclopentyl)oxazolidin-2-one

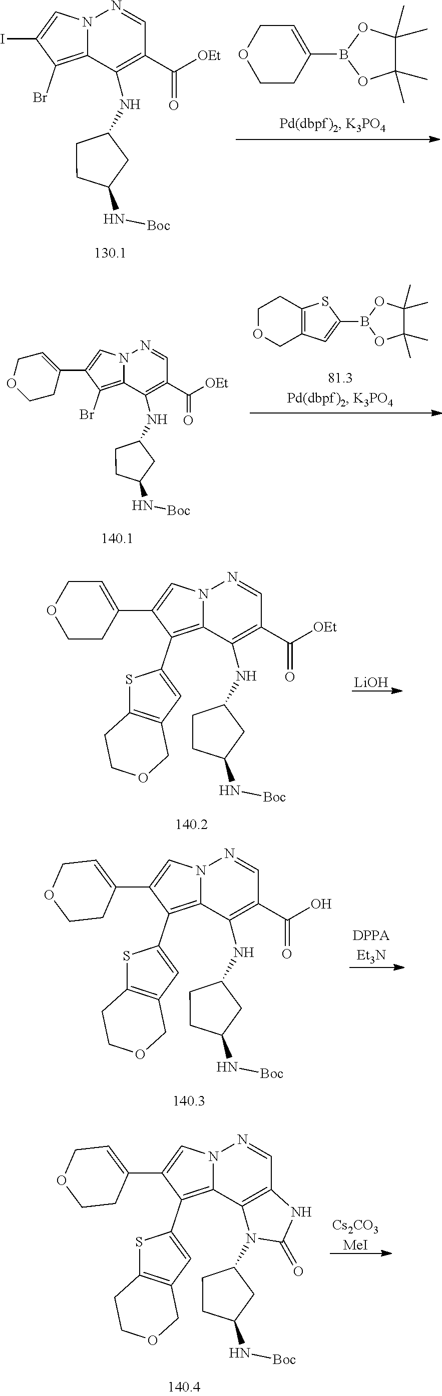

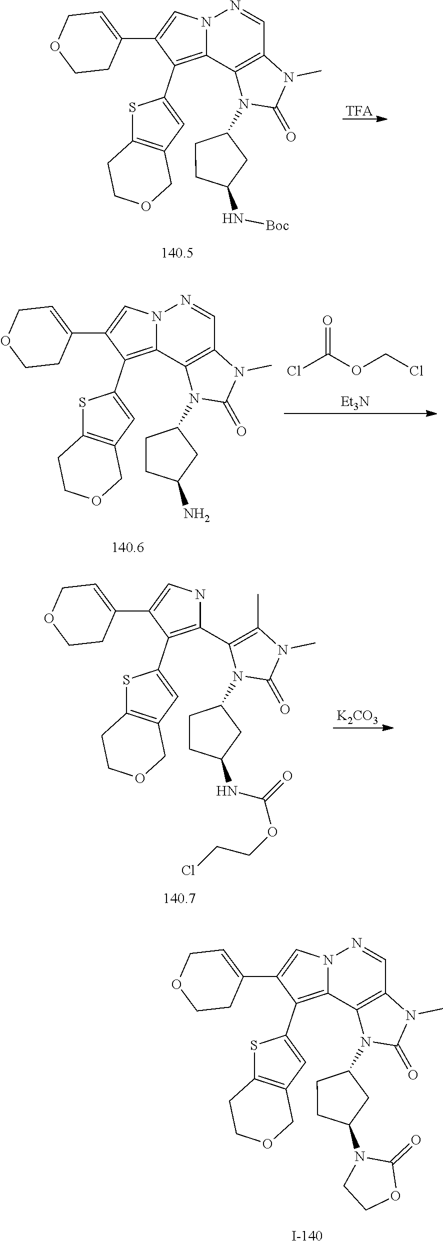

Synthesis of compound 140.1. Compound 140.1 was prepared from compound 130.1 and 2-(3,6-dihydro-2H-pyran-4-yl)-4,4,5,5-tetramethyl-1,3,2-dioxaborolane following the procedure described in the synthesis of compound 54.3. The product was purified by flash column chromatography on silica gel (55% ethyl acetate in hexane). MS(ES): m/z 549.52 and 551.52 [M+H]⁺.

Synthesis of compound 140.2. Compound 140.2 was prepared from compound 140.1 and 81.3 following the procedure described in the synthesis of compound 59.12. The product was purified by flash column chromatography on silica gel (55% ethyl acetate in hexane). MS(ES): m/z 609.70 [M+H]⁺.

Synthesis of compound 140.3. Compound 140.3 was prepared from compound 140.2 following the procedure described in the synthesis of compound 1.10. The product was triturated with pentane, dried under vacuum and used in the next step without purification. MS(ES): m/z 581.68 [M+H]⁺.

Synthesis of compound 140.4. Compound 140.4 was prepared from compound 140.3 following the procedure described in the synthesis of compound 1.11. The product was purified by flash column chromatography on silica gel (Combiflash®, 2.1% methanol in DCM). MS(ES): m/z 578.67 [M+H]⁺.

Synthesis of compound 140.5. Compound 140.5 was prepared from compound 140.4 following the procedure described in the synthesis of compound 1.12. The product was purified by flash column chromatography on silica gel (1.8% methanol in DCM). MS(ES): m/z 592.5 [M+H]⁺.

Synthesis of compound 140.6. Compound 140.6 was prepared from compound 140.5 following the procedure described in the synthesis of compound 1.14. The product was used in the next step without purification. MS(ES): m/z 492.58 [M+H]⁺.

Synthesis of compound 140.7. Compound 140.7 was prepared from compound 140.6 and 2-chloroethyl carbonochloridate following the procedure described in the synthesis of compound 9.7. The product was purified by flash column chromatography on silica gel (Combiflash®, 3.5% methanol in DCM). MS(ES): 599.2 [M+H]⁺.

Synthesis of I-140. Compound I-140 was prepared from compound 140.7 following the procedure described in the synthesis of compound I-9. The product was purified by flash column chromatography on silica gel (Combiflash®, 3.2% methanol in DCM). MS(ES) 562.61 [M+H]⁺. ¹H-NMR (400 MHz, DMSO-d₆): δ 8.48 (s, 1H), 7.97 (s, 1H), 6.89 (s, 1H), 5.71 (s, 1H), 4.70 (s, 1H), 4.67-4.58 (m, 1H), 4.43 (s, 1H), 4.30-4.25 (m, 2H), 4.06 (s, 2H), 4.06-3.98 (m, 2H), 3.74 (t, J=5.6 Hz, 2H), 3.46 (s, 1H), 3.41 (s, 3H), 2.89 (s, 2H), 2.58 (s, 3H), 2.31 (s, 2H), 2.06 (s, 2H), 1.86-1.83 (m, 1H), 1.67 (s, 1H), 1.60-1.54 (m, 1H).

### Example I-141: N-methyl-N-(((1r,3r)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclobutyl)methyl)propionamide

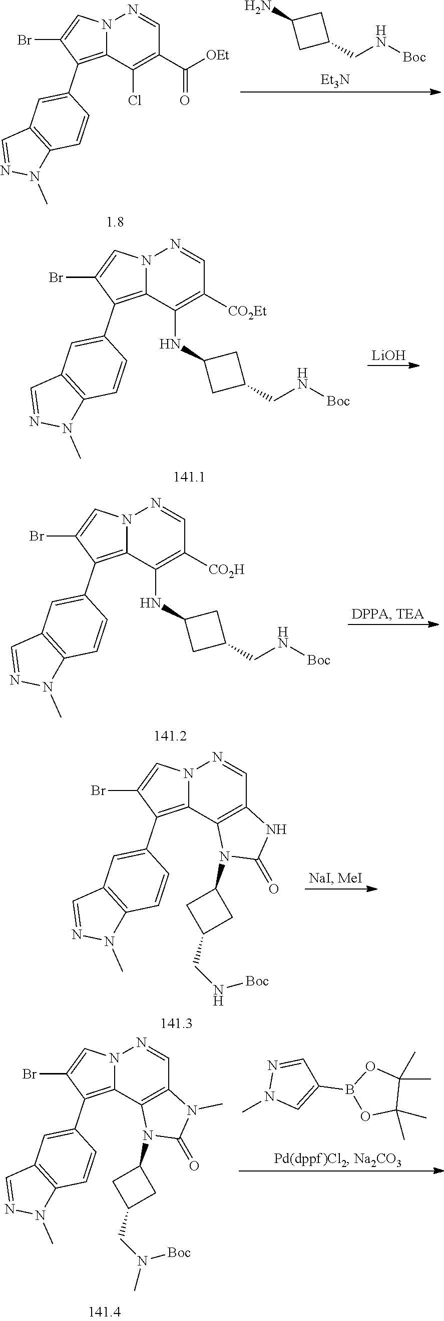

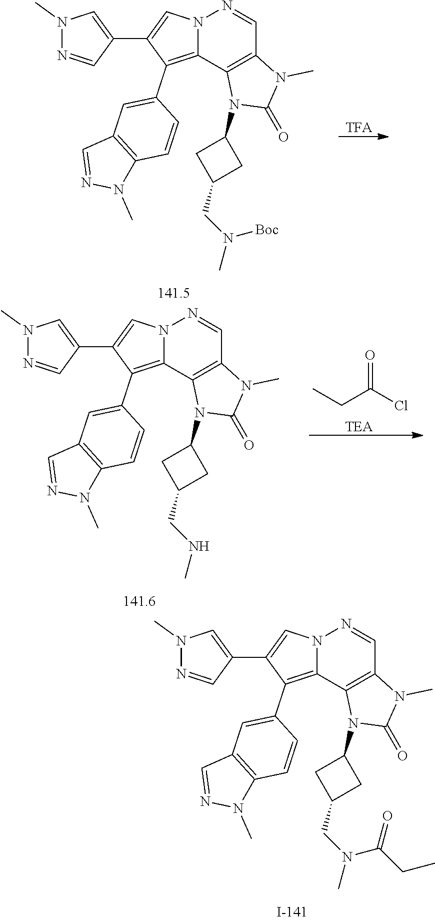

Synthesis of compound 141.1. Compound 141.1 was prepared from compound 1.8 and tert-butyl (((1r,3r)-3-aminocyclobutyl)methyl)carbamate following the procedure described in the synthesis of compound 1.9. The product was purified by flash column chromatography on silica gel (Combiflash®, 50% ethyl acetate in hexane). MS(ES): m/z 597.4 and 599.3 [M+H]⁺.

Synthesis of compound 141.2. Compound 141.2 was prepared from compound 141.1 following the procedure described in the synthesis of compound 1.10. The product was triturated with pentane, dried under vacuum and used in the next step without purification. MS(ES): m/z 569.20 and 571.20 [M+H]⁺.

Synthesis of compound 141.3. Compound 141.3 was prepared from compound 141.2 following the procedure described in the synthesis of compound 1.11. The product was purified by flash column chromatography on silica gel (Combiflash®, 1.8% methanol in DCM). MS(ES): m/z 566.11 & 568.31 [M+H]⁺.

Synthesis of compound 141.4. To a solution of 141.3 (0.300 g, 0.529 mmol, 1.0 equiv) in DMF (8 mL) was added sodium hydride (0.211 g, 5.29 mmol, 10 equiv) and methyl iodide (0.375 g, 2.64 mmol, 5.0 equiv) at 0° C. The reaction mixture was stirred at room temperature for 15 min. It was transferred into ice-water and extracted with ethyl acetate. The combined organic layers were washed with brine, dried over sodium sulfate, filtered and concentrated under reduced pressure. The residue was purified by flash column chromatography on silica gel (1.4% methanol in DCM) to afford 141.4 (0.280 g, 89%). MS(ES): m/z 594.30 and 596.30 [M+H]⁺.

Synthesis of compound 141.5. Compound 141.5 was prepared from compound 141.4 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (Combiflash®, 3.2% methanol in DCM). MS(ES): m/z 596.25 [M+H]⁺.

Synthesis of compound 141.6. Compound 141.6 was prepared from compound 141.5 following the procedure described in the synthesis of compound 1.14. The product was triturated with pentane and dried under reduced pressure. It was used in the next step without purification. MS(ES): 496.44 [M+H]⁺.

Synthesis of compound I-141. To a solution of 141.6 (0.065 g, 0.131 mmol, 1.0 equiv) and triethylamine (0.055 mL, 0.393 mmol, 3.0 equiv) in THF (2.0 mL) was added propionyl chloride (0.025 g, 0.262 mmol, 2.0 equiv) at 0° C. The reaction mixture was stirred at room temperature for 1 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 3% methanol in DCM) to afford I-141 (0.020 g, 28%). MS(ES): 552.30 [M+H]⁺. ¹H-NMR (400 MHz, DMSO-d₆): δ 8.40 (d, J=6.0 Hz, 1H), 8.13-8.09 (m, 2H), 7.87 (d, J=8 Hz, 1H), 7.79-7.76 (m, 1H), 7.43-7.39 (m, 1H), 7.27 (s, 1H), 6.95 (d, J=13.2 Hz, 1H), 4.13 (s, 3H), 3.88-3.83 (m, 1H), 3.67 (s, 3H), 3.36 (s, 3H), 2.86-2.82 (m, 1H), 2.72-2.67 (m, 1H), 2.46 (s, 3H), 2.33-2.27 (m, 1H), 2.19-2.15 (m, 3H), 1.94 (m, 2H), 1.33 (m, 1H), 0.92 (m, 3H).

### Example I-142: methyl ((1R,3R)-3-(8-(3,6-dihydro-2H-pyran-4-yl)-9-(5-ethyl-2-oxo-1,2-dihydropyridin-3-yl)-3-methyl-2-oxo-2,3-dihydro-1H-imidazo[4,5-d]pyrrolo[1,2-b]pyridazin-1-yl)cyclopentyl)carbamate

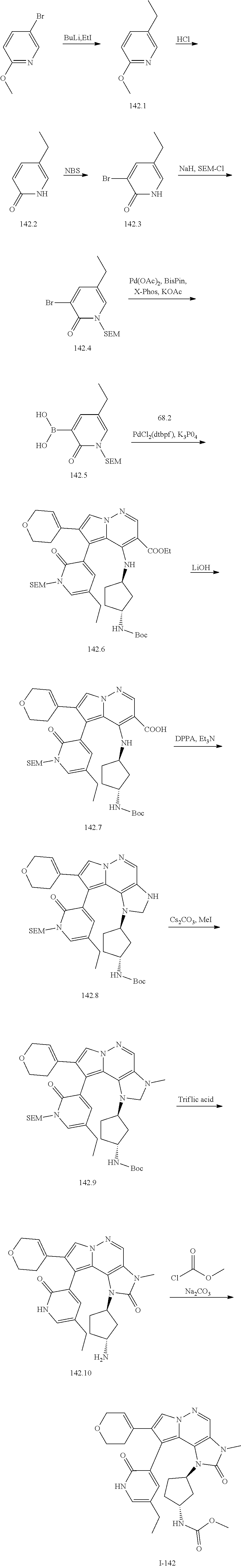

Synthesis of compound 142.1. To a solution of 5-bromo-2-methoxypyridine (50.0 g, 265.95 mmol, 1.0 equiv) in THF (1000 mL) at −78° C. was added n-BuLi (117.02 mL, 292.55 mmol, 1.1 equiv) and stirred for 1.3 h. To the mixture was added ethyl iodide (62.23 g, 398.93 mmol, 1.5 equiv) at −78° C. dropwise in 20 min. The reaction mixture was allowed to warm to room temperature and stirred for 30 min. It was quenched with ice-water and extracted with DCM. The combined organic layers were washed with brine, dried over anhydrous sodium sulfate, filtered and concentrated under reduced pressure. The residue was purified by flash column chromatography on silica gel (10% ethyl acetate in hexane) to afford 142.1 (14.50 g, 40%). MS(ES): m/z 138.30 [M+H]⁺.

Synthesis of compound 142.2. A mixture of 142.1 (14.50 g, 105.83 mmol, 1.0 equiv) and concentrated HCl (217.5 mL) was stirred at 100° C. for 16 h. It was cooled to rt and quenched with NaHCO₃ solution and extracted with 10% MeOH/DCM solution. The combined organic layers were washed with brine, dried over anhydrous sodium sulfate, filtered and concentrated under reduced pressure. The residue was purified by flash column chromatography on silica gel (Combiflash®, 10% methanol in DCM) to afford 142.2 (4.2 g, 18%). MS(ES): m/z 124.15 [M+H]⁺.

Synthesis of compound 142.3. To a solution of 142.2 (4.2 g, 34.14 mmol, 1.0 equiv) in DCM (84 mL) was added N-bromosuccinimide (6.07 g, 34.14 mmol, 1.0 equiv) at 0° C. in small portions. The reaction mixture was stirred at room temperature for 30 min. It was quenched with a saturated aqueous solution of sodium thiosulphate and extracted with DCM. The combined organic layers were washed with brine, dried over anhydrous sodium sulfate, filtered and concentrated under reduced pressure. The residue was purified by flash column chromatography on silica gel (10% MeOH in DCM) to afford 142.3 (4.2 g, 61%). MS(ES): m/z 202 and 204 [M+H]⁺.

Synthesis of compound 142.4. To a solution of 142.3 (4.2 g, 21.00 mmol, 1.0 equiv) in DMF (42 mL) at 0° C. was added sodium hydride (60% dispersion in mineral oil, 1.26 g, 31.5 mmol, 1.5 equiv) and stirred for 30 min. To the mixture was added SEM-Cl and stirred for 30 min. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 50% ethyl acetate in hexane) to afford 142.4 (1.5 g, 22%). MS(ES): m/z 332.20 and 334.10 [M+H]⁺.

Synthesis of compound 142.5. A mixture of 142.4 (1.5 g, 4.51 mmol, 1.0 equiv), bis(pinacolato)diboron (5.73 g, 22.59 mmol, 5.0 equiv), potassium acetate (0.88 g, 9.03 mmol, 2.0 equiv) and dicyclohexyl[2′,4′,6′-tris(propan-2-yl)[1,1′-biphenyl]-2-yl]phosphane (0.43 g, 0.90 mmol, 0.2 equiv) in 1,4 dioxane was degassed by bubbling through a stream of argon for 10 min. Palladium (II) acetate (0.080 g, 0.36 mmol, 0.1 equiv) was added and degassed for 5 min and the reaction mixture was stirred at 100° C. for 1 h. It was cooled to room temperature, filtered through a pad of Celite® and rinsed with ethyl acetate. The filtrate was concentrated under reduced pressure to afford 142.5 (1.2 g). MS(ES): m/z 298.5 [M+H]⁺. The residue was used in the next step without purification.

Synthesis of compound 142.6. Compound 142.6 was prepared from compound 142.5 and 68.2 following the procedure described in the synthesis of compound 59.12. The product was purified by flash column chromatography on silica gel (Combiflash®, 0-3.2% methanol in DCM). MS(ES): m/z 722.3 [M+H]⁺.

Synthesis of compound 142.7. Compound 142.7 was prepared from compound 142.6 following the procedure described in the synthesis of compound 1.10. The product was triturated with pentane, dried under vacuum and used in the next step without purification. MS (ES): m/z 694.20 [M+H]⁺.

Synthesis of compound 142.8. Compound 142.8 was prepared from compound 142.7 following the procedure described in the synthesis of compound 1.11. The product was purified by flash column chromatography on silica gel (Combiflash®, 1.8% methanol in DCM). MS(ES): m/z 691.31 [M+H]⁺.

Synthesis of compound 142.9. Compound 142.9 was prepared from compound 142.8 following the procedure described in the synthesis of compound 1.12. The product was purified by flash column chromatography on silica gel (1.4% methanol in DCM). MS(ES): m/z 705.20 [M+H]⁺.

Synthesis of compound 142.10. Compound 142.10 was prepared from compound 142.9 following the procedure described in the synthesis of compound 21.8. The product was triturated with pentane and dried under reduced pressure. It was used in the next step without purification. MS(ES): m/z 475.2 [M+H]⁺.

Synthesis of compound I-142. Compound I-142 was prepared from compound 142.10 following the procedure described in the synthesis of compound I-1. The product was purified by flash column chromatography on silica gel (Combiflash®, 2.6% methanol in DCM). MS(ES): m/z 533.3 [M+H]⁺. ¹H-NMR (400 MHz, DMSO-d₆): δ 11.30 (s, 1H), 8.32 (s, 1H), 7.83 (d, J=3.6 Hz, 1H), 7.35 (s, 1H), 7.21 (d, J=16.8 Hz, 1H), 6.45 (s, 1H), 5.67 (d, J=14.4 Hz, 1H), 4.51-4.49 (m, 1H), 4.18-4.15 (m, 1H), 4.13-4.05 (m, 2H), 3.80-3.73 (m, 2H), 3.56 (s, 3H), 3.37 (s, 3H), 2.41-2.40 (m, 3H), 2.38-2.33 (m, 1H), 2.17-2.14 (m, 2H), 2.11-2.09 (m, 2H), 1.57-1.54 (m, 2H), 1.12-1.10 (m, 3H).

### Example I-143: 3-((1S,3S)-3-(9-(2,3-dihydrobenzo[b][1,4]dioxin-6-yl)-8-(3-methoxy-1-methyl-1H-pyrazol-4-yl)-3-methyl-2-oxo-2,3-dihydro-1H-imidazo[4,5-d]pyrrolo[1,2-b]pyridazin-1-yl)cyclopentyl)oxazolidin-2-one

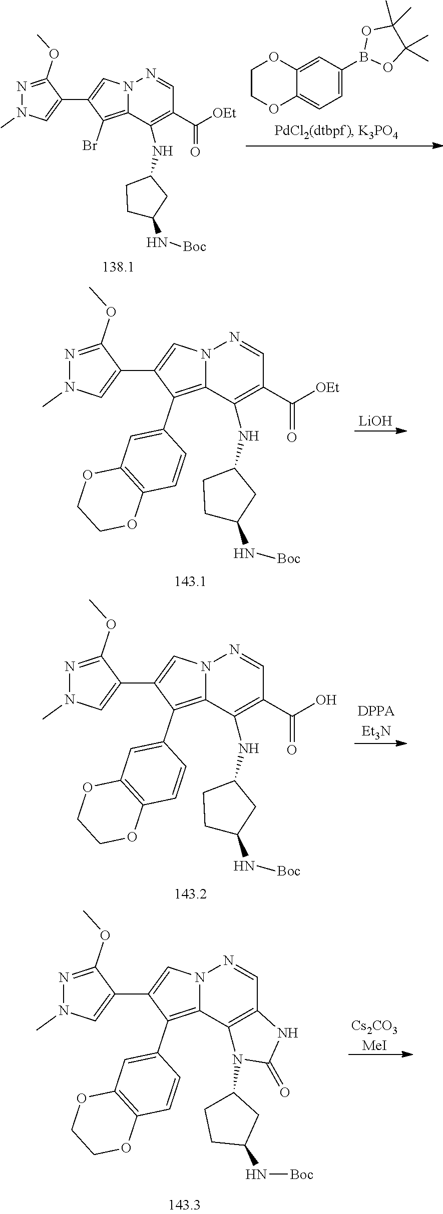

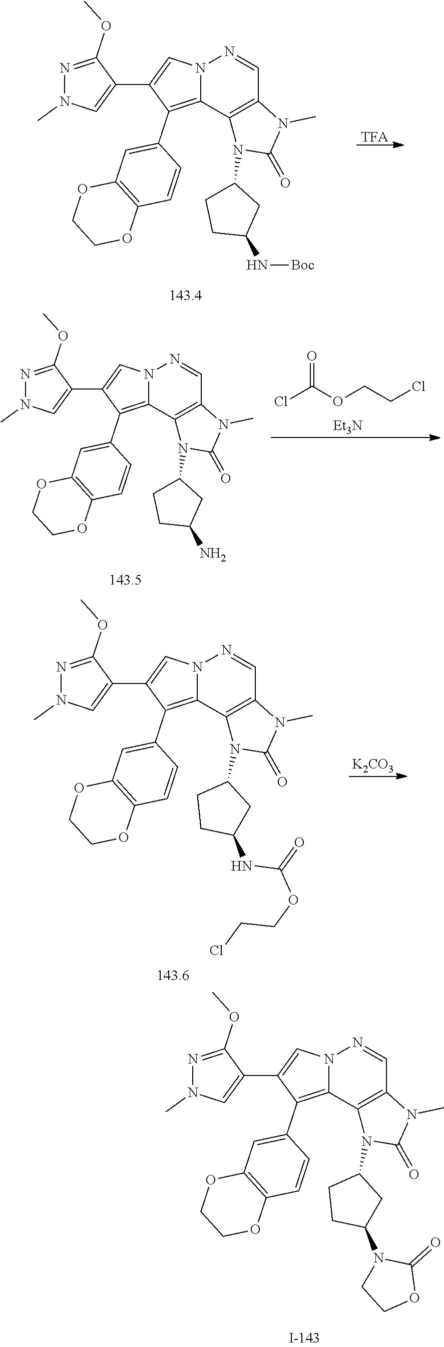

Synthesis of compound 143.1. Compound 143.1 was prepared from compound 138.1 and 2-(2,3-dihydrobenzo[b][1,4]dioxin-6-yl)-4,4,5,5-tetramethyl-1,3,2-dioxaborolane following the procedure described in the synthesis of compound 59.12. The product was purified by flash column chromatography on silica gel (1.1% methanol in DCM). MS(ES): m/z 633.5 [M+H]⁺.

Synthesis of compound 143.2. Compound 143.2 was prepared from compound 143.1 following the procedure described in the synthesis of compound 1.10. The product was triturated with pentane, dried under vacuum and used in the next step without purification. MS(ES): m/z 605.3 [M+H]⁺.

Synthesis of compound 143.3. Compound 143.3 was prepared from compound 143.2 following the procedure described in the synthesis of compound 1.11. The product was purified by flash column chromatography on silica gel (Combiflash®, 1.8% methanol in DCM). MS(ES): m/z 602.6 [M+H]⁺.

Synthesis of compound 143.4. Compound 143.4 was prepared from compound 143.3 following the procedure described in the synthesis of compound 1.12. The product was purified by flash column chromatography on silica gel (1.4% methanol in DCM). MS(ES): m/z 616.3 [M+H]⁺.

Synthesis of compound 143.5. Compound 143.5 was prepared from compound 143.4 following the procedure described in the synthesis of compound 1.14. The product was used in the next step without purification. MS(ES): m/z 516.55 [M+H]⁺.

Synthesis of compound 143.6. Compound 143.6 was prepared from compound 143.5 and 2-chloroethyl carbonochloridate following the procedure described in the synthesis of compound 9.7. The product was used in the next step without purification. MS(ES) m/z: 623.5 [M+H]⁺.

Synthesis of I-143. Compound I-143 was prepared from compound 143.6 following the procedure described in the synthesis of compound I-9. The product was purified by flash column chromatography on silica gel (Combiflash®, 2.8% methanol in DCM). MS(ES): m/z 586.4 [M+H]⁺. ¹H-NMR (400 MHz, DMSO-d₆): δ 8.37 (s, 1H), 8.01 (s, 1H), 7.06-6.97 (m, 1H), 6.86-6.84 (m, 2H), 6.45-6.35 (m, 1H), 4.33-4.25 (m, 8H), 3.98 (s, 3H), 3.74 (m, 1H), 3.55 (s, 3H), 2.54 (s, 3H), 2.05 (m, 2H), 1.78-1.77 (m, 1H), 1.35-1.18 (m, 2H), 1.39-1.18 (m, 2H).

### Example I-144: methyl ((1R,3R)-3-(9-(7-fluoro-3,3-dimethyl-2-oxoindolin-6-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)carbamate

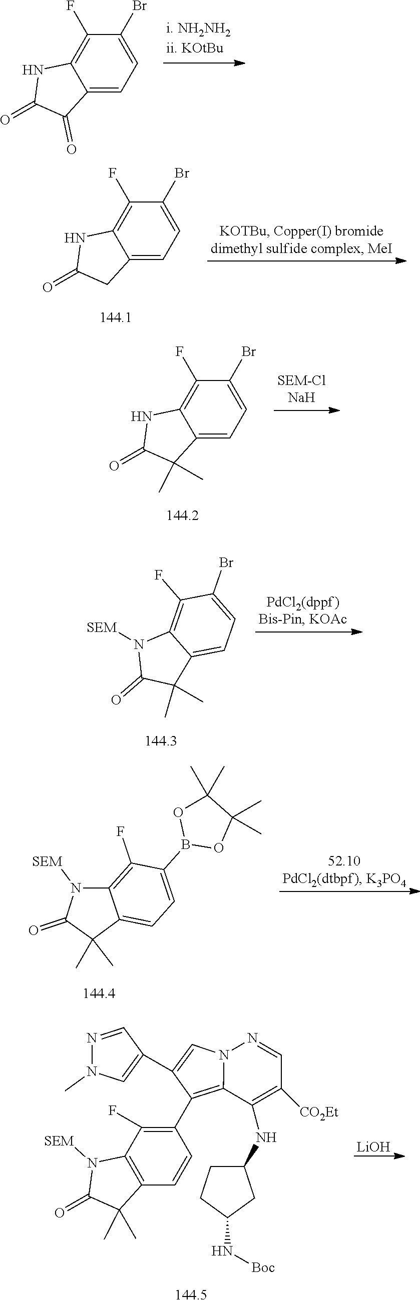

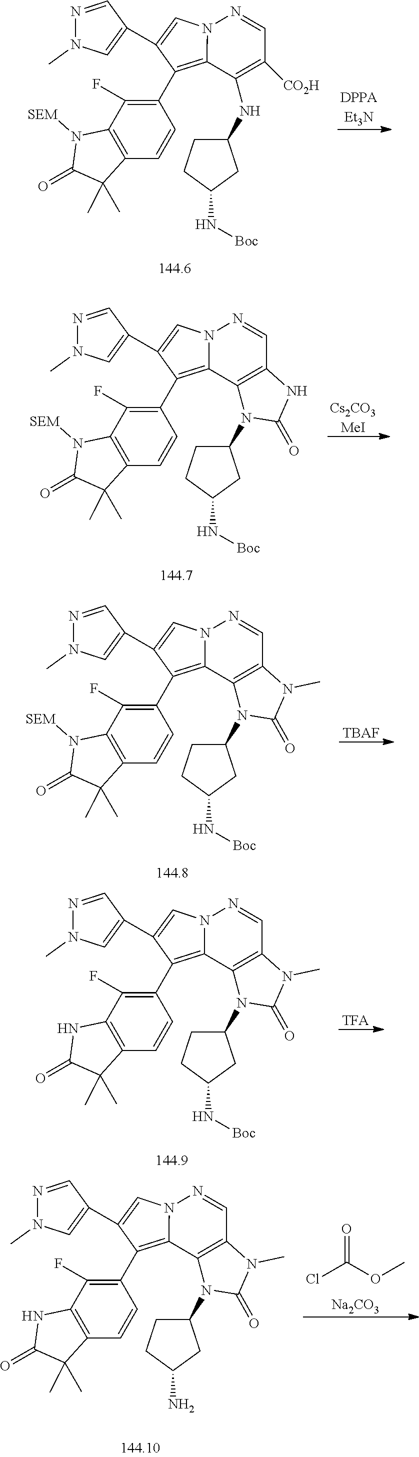

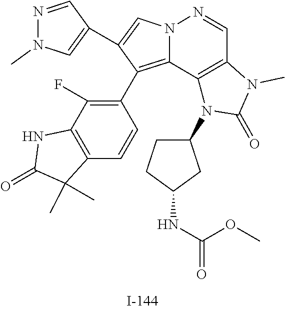 [SMILES: COC(=O)N[C@@H]1CC[C@@H](n2c(=O)n(C)c3cnn4cc(-c5cnn(C)c5)c(-c5ccc6c(c5F)NC(=O)C6(C)C)c4c32)C1]

Synthesis of compound 144.1 To a solution of 6-bromo-7-fluoroindoline-2,3-dione (20 g, 81.96 mmol, 1.0 equiv) in ethanol (360 mL) was added hydrazine hydrate (3.5 g, 110.65 mmol, 1.35 equiv). The reaction mixture was stirred at 80° C. for 0.5 h. Yellow precipitate was formed and collected by filtration. The yellow precipitate was dissolved in anhydrous ethanol (360 mL) and was added potassium tert-butoxide (32.13 g, 286.88 mmol, 3.5 equiv). The mixture was stirred at 80° C. for 2 h. It was acidified with 1 M HCl to pH ˜2 and extracted with ethyl acetate. The combined organic phase was washed with water, brine, dried with anhydrous sodium sulfate, filtered and concentrated under reduced pressure. The residue was purified by flash column chromatography on silica gel (Combiflash®, 30% ethyl acetate in n-hexane) to afford 144.1 (11.0 g, 58%), MS (ES): m/z 230 and 232 [M+H]⁺.

Synthesis of compound 144.2. To a solution of potassium tert-butoxide (26.7 g, 239.13 mmol, 5.0 equiv) in THF (110 mL) was added 144.1 (11.0 g, 47.82 mmol, 1.0 equiv) at 0° C. The reaction mixture was stirred at room temperature for 1 h. Copper (I) bromide dimethyl sulfide complex (0.980 g, 4.782 mmol, 0.1 equiv) and methyl iodide (6.79 g, 47.82 mmol, 1.0 equiv) were added to the reaction mixture and stirred at room temperature for 16 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (Combiflash®, 30% ethyl acetate in n-hexane) to afford 144.2 (3.5 g, 28%). MS (ES): m/z 258.3 and 260.3 [M+H]⁺.

Synthesis of compound 144.3. To a solution of 144.2 (3.5 g, 13.56 mmol, 1.0 equiv) in 30 mL THF was added sodium hydride (60% dispersion in mineral oil, 1.0 g, 27.13 mmol, 2.0 equiv) in small portions at 0° C. and stirred for 30 min. To the suspension was added 2-(trimethylsilyl) ethoxymethyl chloride (3.3 g, 20.34 mmol, 1.5 equiv) The reaction mixture was stirred at room temperature for 2 h. It was quenched with ice-water and extract with ethyl acetate. The combined organic layers were washed with brine, dried over anhydrous sodium sulfate, filtered and concentrated under reduced pressure. The residue was purified by flash column chromatography on silica gel (Combiflash®, 17% ethyl acetate in hexane) to afford 144.3 (2.2 g, 42%) MS (ES): m/z 388.2 and 390.2 [M+H]⁺.

Synthesis of compound 144.4. A mixture of 144.3 (2.2 g, 5.67 mmol, 1.0 equiv), bis(pinacolato)diborane (2.15 g, 8.50 mmol, 1.5 equiv) and potassium acetate (1.4 g, 14.17 mmol, 2.5 equiv) in 1,4-dioxane (20 mL) was degassed by bubbling through a stream of argon for 10 min. 1,1′-Bis(diphenylphosphino)ferrocene) dichloropalladium-DCM (0.414 g, 0.567 mmol, 0.1 equiv) was added to the reaction mixture and degassed for 10 min. The mixture was stirred at 100° C. for 12 h. It was poured into water and extracted with ethyl acetate. The combined organic layers were washed with brine, dried over anhydrous sodium sulfate, filtered and concentrated under reduced pressure. The residue was purified by flash column chromatography on silica gel (Combiflash®, 20% ethyl acetate in hexane) to afford 144.4 (1.1 g, 45%). MS(ES): m/z 436.6 [M+H]⁺.

Synthesis of compound 144.5. Compound 144.5 was prepared from compound 144.4 and 52.10 following the procedure described in the synthesis of compound 59.12. The product was purified by flash column chromatography on silica gel (Combiflash®, 40 ethyl acetate in hexane). MS(ES): m/z 777.3 [M+H]⁺.

Synthesis of compound 144.6. Compound 144.6 was prepared from compound 144.5 following the procedure described in the synthesis of compound 1.10. The product was triturated with pentane, dried under vacuum and used in the next step without purification. MS(ES): m/z 748.8 [M+H]⁺.

Synthesis of compound 144.7. Compound 144.7 was prepared from compound 144.6 following the procedure described in the synthesis of compound 1.11. The product was purified by flash column chromatography on silica gel (Combiflash®, 2.3% methanol in DCM). MS(ES): m/z 745.4 [M+H]⁺.

Synthesis of compound 144.8. Compound 144.8 was prepared from compound 144.7 following the procedure described in the synthesis of compound 1.12. The product was purified by flash column chromatography on silica gel (1.8% methanol in DCM). MS(ES): m/z 759.5 [M+H]⁺.

Synthesis of compound 144.9. To a solution of 144.8 (0.125 g, 0.164 mmol, 1.0 equiv) in THF (3.0 mL) at 0° C. was added dropwise tetra-n-butyl ammonium fluoride (0.5 mL). The mixture was stirred at 80° C. for 16 h. It was transferred into a mixture of ice and aqueous sodium bicarbonate and extracted with ethyl acetate. The combined organic layers were washed with brine, dried over anhydrous sodium sulfate, filtered and concentrated under reduced pressure to afford compound 144.9 (0.060 g, 58%). MS(ES): m/z 629.6 [M+H]⁺.

Synthesis of compound 144.10. Compound 144.10 was prepared from compound 144.9 following the procedure described in the synthesis of compound 1.14. The product was used in the next step without purification. MS(ES): m/z 529.3 [M+H]⁺.

Synthesis of I-144. Compound I-144 was prepared from compound 144.10 following the procedure described in the synthesis of compound I-1. The product was purified by flash column chromatography on silica gel (Combiflash®, 3.3% methanol in DCM). MS(ES): m/z 587.43 [M+H]⁺. ¹H-NMR (400 MHz, DMSO-d₆): δ 10.95-10.83 (m, 1H), 8.44 (d, J=2.4 Hz, 1H), 8.18-8.18 (m, 1H), 7.28-0.23 (m, 2H), 7.11-6.99 (m, 2H), 6.55-6.43 (m, 1H), 4.13-4.07 (m, 2H), 3.72 (s, 3H), 3.51 (s, 3H), 3.38 (s, 3H), 1.98-1.93 (m, 2H), 1.58-1.38 (m, 6H), 1.38-1.26 (m, 2H), 0.96-0.78 (m, 2H).

### Example I-145: methyl ((1R,3R)-3-(8-(3,6-dihydro-2H-pyran-4-yl)-3-methyl-2-oxo-9-(3-oxo-3,4-dihydro-2H-benzo[b][1,4]oxazin-6-yl)-2,3-dihydro-1H-imidazo[4,5-d]pyrrolo[1,2-b]pyridazin-1-yl)cyclopentyl)carbamate

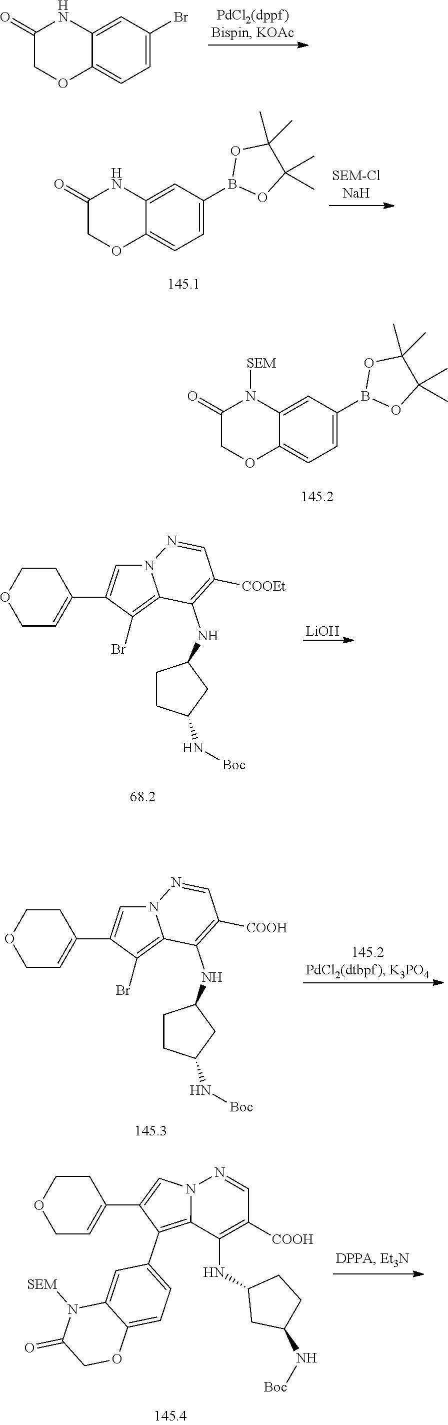

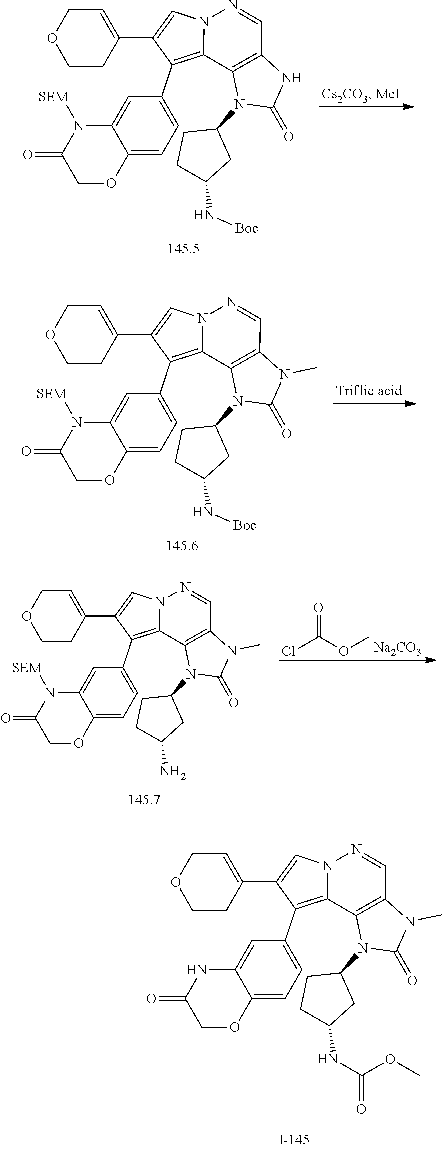

Synthesis of compound 145.1. A mixture of 6-bromo-2H-benzo[b][1,4]oxazin-3(4H)-one (5.0 g, 21.9 mmol, 1.0 equiv), bis(pinacolato)diboron (8.3 g, 32.8 mmol, 1.5 equiv) and potassium acetate (5.4 g, 54.7 mmol, 2.5 equiv) in 1,4-dioxane (50 mL) was degassed by bubbling through a stream of argon for 10 min. [1,1′-Bis(diphenylphosphino)ferrocene]dichloropalladium (II) (1.6 g, 2.19 mmol, 0.1 equiv) was added and degassed for 5 min. The reaction mixture was stirred at 100° C. for 3 h. It was cooled to room temperature, transferred into ice-water and extracted with ethyl acetate. The combined organic layers were washed with brine, dried over anhydrous sodium sulfate, filtered and concentrated under reduced pressure. The residue was purified by flash column chromatography on silica gel (22% ethyl acetate in hexane) to afford 145.1 (5.00 g, 82%). MS(ES): m/z 276.20 [M+H]⁺.

Synthesis of compound 145.2. To a solution of 145.1 (5 g, 18.18 mmol, 1.0 equiv) in DMF (50 mL) was added in small portions sodium hydride (60% dispersion in mineral oil, 1.7 g, 36.6 mmol, 2.0 equiv) at 0° C. and stirred at 0° C. for 1 h. 2-(trimethylsilyl) ethoxy methyl chloride (SEM-Cl) (4.5 g, 27.2 mmol, 1.5 equiv) was added and the reaction mixture was stirred at 0° C. for 2 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (12% ethyl acetate in hexane) to afford 145.2 (6.00 g, 82%). ¹H-NMR (400 MHz, CDCl₃): δ 7.72 (s, 1H), 7.50 (dd, J=1.2, 8.0 Hz, 1H), 6.99 (d, J=8.0 Hz, 1H), 5.40 (s, 2H), 4.64 (s, 2H), 3.72-3.68 (m, 2H), 1.34 (s, 12H), 0.96 (s, 2H), 0.03-−0.01 (m, 9H).

Synthesis of compound 145.3. Compound 145.3 was prepared from compound 68.2 following the procedure described in the synthesis of compound 1.10. The product was triturated with pentane, dried under vacuum and used in the next step without purification. MS(ES): m/z 521.14 and 523.14 [M+H]⁺.

Synthesis of compound 145.4. Compound 145.4 was prepared from compound 145.2 and 145.3 following the procedure described in the synthesis of compound 59.12. The product was purified by flash column chromatography on silica gel (35% ethyl acetate in hexane). MS(ES): m/z 720.89 [M+H]⁺.

Synthesis of compound 145.5. Compound 145.5 was prepared from compound 145.4 following the procedure described in the synthesis of compound 1.11. The product was purified by flash column chromatography on silica gel (Combiflash®, 2.1% methanol in DCM). MS(ES): m/z 717.89 [M+H]⁺.

Synthesis of compound 145.6. Compound 145.6 was prepared from compound 145.5 following the procedure described in the synthesis of compound 1.12. The product was purified by flash column chromatography on silica gel (1.5% methanol in DCM). MS(ES): m/z 731.91 [M+H]⁺.

Synthesis of compound 145.7. Compound 145.7 was prepared from compound 145.6 following the procedure described in the synthesis of compound 21.8. The product was triturated with pentane and dried under reduced pressure. It was used in the next step without purification. MS(ES): m/z 501.43 [M+H]⁺.

Synthesis of compound I-145. Compound I-145 was prepared from compound 145.7 following the procedure described in the synthesis of compound I-1. The product was purified by flash column chromatography on silica gel (Combiflash®, 3.8% methanol in DCM). MS(ES): m/z 559.27 [M+H]⁺. ¹H-NMR (400 MHz, DMSO-d₆): δ 10.52 (s, 1H), 8.33 (d, J=3.2 Hz, 1H), 7.85 (d, J=3.6 Hz, 1H), 7.03 (d, J=7.6 Hz, 1H), 6.93-6.88 (m, 2H), 6.47 (s, 1H), 5.50 (s, 1H), 4.68-4.59 (m, 2H), 4.07-4.05 (m, 3H), 4.01-4.00 (m, 1H), 3.69-3.66 (m, 1H), 3.52 (s, 3H), 3.36 (s, 3H), 2.18 (s, 4H), 1.99-1.93 (m, 2H), 1.53-1.50 (m, 1H), 1.32-1.26 (m, 1H), 0.96 (m, 1H).

### Example I-146: N-(((1r,3r)-3-(9-(6,7-dihydro-4H-thieno[3,2-c]pyran-2-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclobutyl)methyl)-N-methylpropionamide

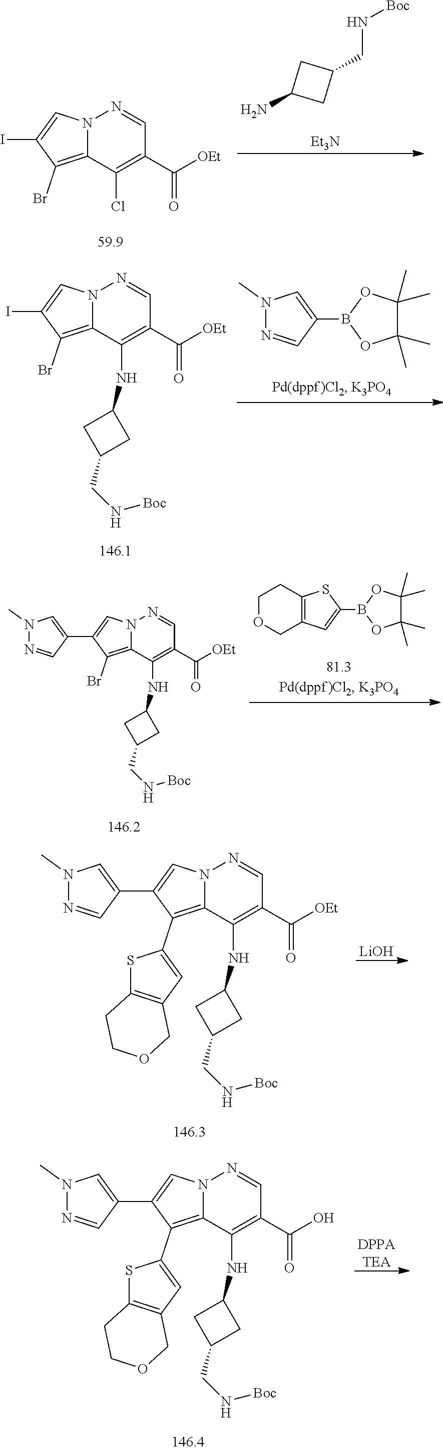

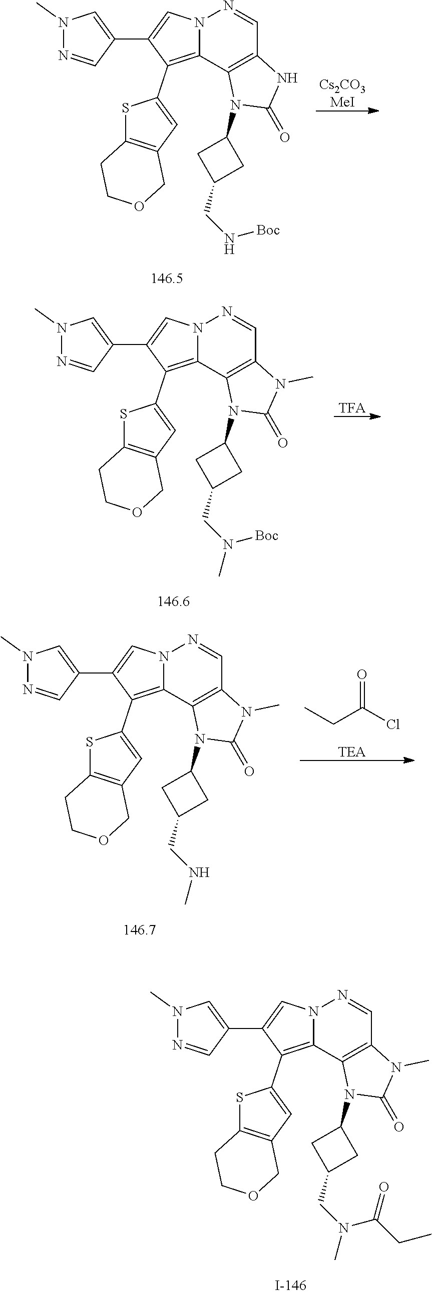

Synthesis of compound 146.1. Compound 146.1 was prepared from compound 59.9 and tert-butyl (((1r,3r)-3-aminocyclobutyl)methyl)carbamate following the procedure described in the synthesis of compound 1.9. The product was purified by flash column chromatography on silica gel (35% ethyl acetate in hexane). MS(ES): m/z 593.2 and 595.2 [M+H]⁺.

Synthesis of compound 146.2. Compound 146.2 was prepared from compound 146.1 and 1-methyl-4-(4,4,5,5-tetramethyl-1,3,2-dioxaborolan-2-yl)-1H-pyrazole following the procedure described in the synthesis of compound 59.12. The product was purified by flash column chromatography on silica gel (55% ethyl acetate in hexane). MS(ES): m/z 547.52 and 549.52 [M+H]⁺.

Synthesis of compound 146.3. Compound 146.3 was prepared from compound 146.2 and 81.3 following the procedure described in the synthesis of compound 59.12. The product was purified by flash column chromatography on silica gel (55% ethyl acetate in hexane). MS(ES): m/z 607.53 [M+H]⁺.

Synthesis of compound 146.4. Compound 146.4 was prepared from compound 146.3 following the procedure described in the synthesis of compound 1.10. The product was triturated with pentane, dried under vacuum and used in the next step without purification. MS(ES): m/z 579.68 [M+H]⁺.

Synthesis of compound 146.5. Compound 146.5 was prepared from compound 146.4 following the procedure described in the synthesis of compound 1.11. The product was purified by flash column chromatography on silica gel (Combiflash®, 2.1% methanol in DCM). MS(ES): m/z 576.67 [M+H]⁺.

Synthesis of compound 146.6. Compound 146.6 was prepared from compound 146.5 following the procedure described in the synthesis of compound 1.12. The product was purified by flash column chromatography on silica gel (1.8% methanol in DCM). MS(ES): m/z 604.5 [M+H]⁺.

Synthesis of compound 146.7. Compound 146.7 was prepared from compound 146.6 following the procedure described in the synthesis of compound 1.14. The product was used in the next step without purification. MS(ES): m/z 504.58 [M+H]⁺.

Synthesis of compound I-146. Compound I-146 was prepared from compound 146.7 following the procedure described in the synthesis of compound I-141. The product was purified by flash column chromatography on silica gel (Combiflash®, 3.2% methanol in DCM). MS (ES): m/z 560.28 [M+H]⁺. ¹H-NMR (400 MHz, DMSO-d₆): δ 8.45 (s, 1H), 8.12 (s, 1H), 7.54 (s, 1H), 7.24 (s, 1H), 6.91 (s, 1H), 4.74 (s, 2H), 4.46-4.42 (m, 1H), 3.99-3.97 (m, 2H), 3.79 (s, 3H), 3.34 (s, 3H), 3.29-3.20 (m, 2H), 3.14-2.92 (m, 2H), 2.89 (s, 2H), 2.84 (s, 3H), 2.78 (s, 1H), 2.34-2.30 (m, 2H), 1.67-1.62 (m, 2H), 1.01-0.94 (m, 3H).

### Example I-147: N-((1S,3S)-3-(9-(benzo[d][1,3]dioxol-5-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)propionamide

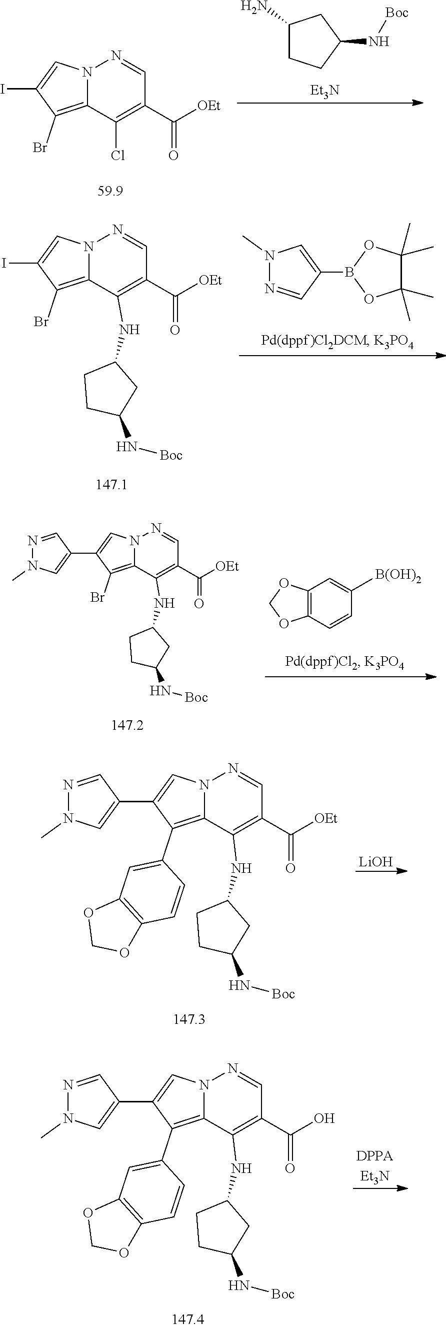

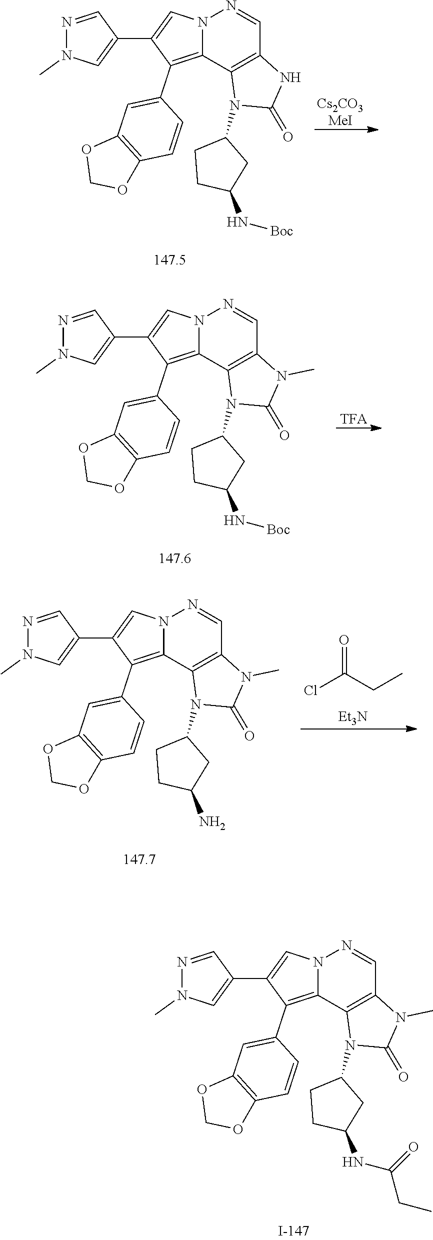

Synthesis of compound 147.1. Compound 147.1 was prepared from compound 59.9 and tert-butyl ((1S,3S)-3-aminocyclopentyl)carbamate following the procedure described in the synthesis of compound 1.9. The product was purified by flash column chromatography on silica gel (22% ethyl acetate in n-hexane). MS (ES): m/z 593.2 and 595.2 [M+H]⁺.

Synthesis of compound 147.2. Compound 147.2 was prepared from compound 147.1 and 1-methyl-4-(4,4,5,5-tetramethyl-1,3,2-dioxaborolan-2-yl)-1H-pyrazole following the procedure described in the synthesis of compound 54.3. The product was purified by flash column chromatography on silica gel (45% ethyl acetate in n-hexane). MS (ES): m/z 547.5 and 549.5 [M+H]⁺.

Synthesis of compound 147.3. Compound 147.3 was prepared from compound 147.2 and benzo[d][1,3]dioxol-5-ylboronic acid following the procedure described in the synthesis of compound 59.12. The product was purified by flash column chromatography on silica gel (40% ethyl acetate in n-hexane). MS (ES): m/z 589.6 [M+H]⁺.

Synthesis of compound 147.4. Compound 147.4 was prepared from compound 147.3 following the procedure described in the synthesis of compound 1.10. The product was used in the next step without purification. MS (ES): m/z 561.6 [M+H]⁺.

Synthesis of compound 147.5. Compound 147.5 was prepared from compound 147.4 following the procedure described in the synthesis of compound 1.11. The product was purified by flash column chromatography on silica gel (2.5% methanol in DCM). MS (ES): m/z 558.6 [M+H]⁺.

Synthesis of compound 147.6. Compound 147.6 was prepared from compound 147.5 following the procedure described in the synthesis of compound 1.12. The product was purified by flash column chromatography on silica gel (2.5% methanol in DCM). MS (ES): m/z 572.6 [M+H]⁺.

Synthesis of compound 147.7. Compound 147.7 was prepared from compound 147.6 following the procedure described in the synthesis of compound 1.14. The product was used in the next step without purification. MS (ES): m/z 472.5 [M+H]⁺.

Synthesis of compound I-147. Compound I-147 was prepared from compound 147.7 following the procedure described in the synthesis of compound I-141. The product was purified by flash column chromatography on silica gel (Combiflash®, 2.8% methanol in DCM). MS (ES): m/z 528.5 [M+H]⁺. ¹H NMR (400 MHz, DMSO-d₆): δ 8.37 (d, J=2.4 Hz, 1H), 8.11 (t, J=2.4 Hz, 1H), 7.51 (dd, J=6.8 Hz, 29.6 Hz, 1H), 7.31 (d, J=3.2 Hz, 1H), 7.15 (d, J=2.4 Hz, 1H), 7.06-7.03 (m, 1H), 6.89 (d, J=1.6 Hz, 1H), 6.87-6.83 (m, 1H), 6.20 (s, 1H), 6.07 (s, 1H), 4.22 (s, 1H), 3.93 (s, 1H), 3.73 (s, 3H), 2.12-2.06 (m, 2H), 2.01 (d, J=7.6 Hz, 3H), 1.93 (d, J=8.0 Hz, 2H), 1.46 (s, 1H), 1.22 (d, J=6.4 Hz, 3H), 0.98 (d, J=2.4 Hz, 3H).

### Example I-148: 3-((1S,3S)-3-(9-(2,3-dihydrobenzofuran-6-yl)-3-methyl-8-(1-methyl-1H-pyrazol-4-yl)-2-oxo-2,3-dihydro-1H-imidazo[4,5-d]pyrrolo[1,2-b]pyridazin-1-yl)cyclopentyl)oxazolidin-2-one

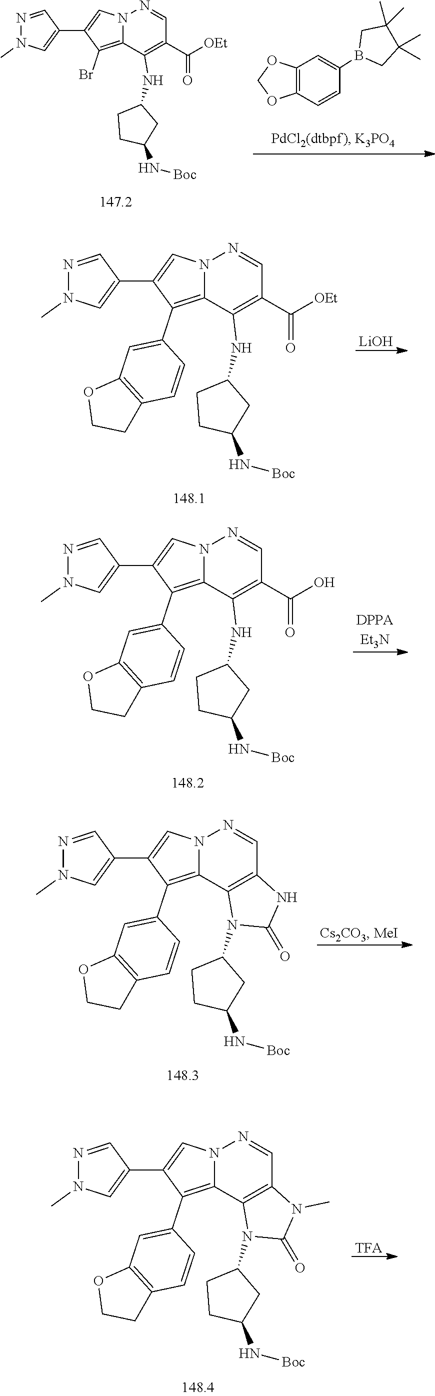

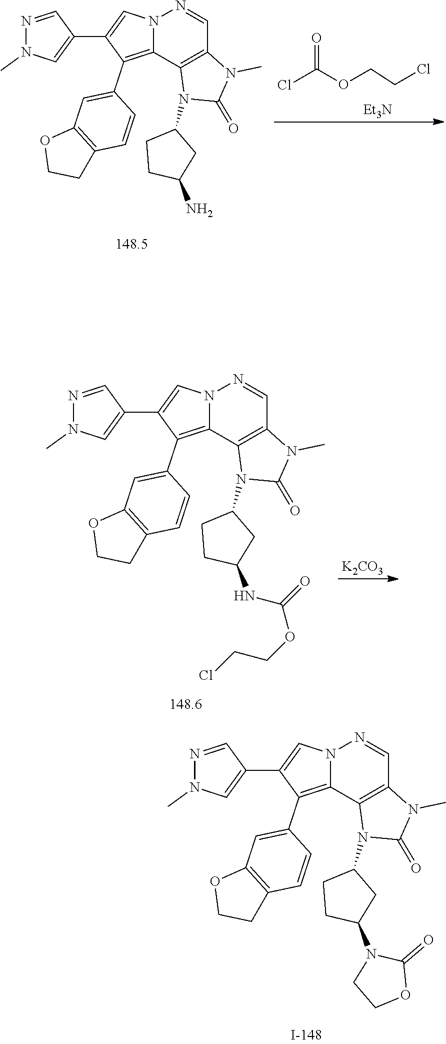

Synthesis of compound 148.1. Compound 148.1 was prepared from compound 147.2 and 2-(2,3-dihydrobenzofuran-6-yl)-4,4,5,5-tetramethyl-1,3,2-dioxaborolane following the procedure described in the synthesis of compound 59.12. The product was purified by flash column chromatography on silica gel (42% ethyl acetate in n-hexane). MS (ES): m/z 587.6 [M+H]⁺.

Synthesis of compound 148.2. Compound 148.2 was prepared from compound 148.1 following the procedure described in the synthesis of compound 1.10. The product was used in the next step without purification. MS (ES): m/z 559.6 [M+H]⁺.

Synthesis of compound 148.3. Compound 148.3 was prepared from compound 148.2 following the procedure described in the synthesis of compound 1.11. The product was purified by flash column chromatography on silica gel (2.5% methanol in DCM). MS (ES): m/z 556.6 [M+H]⁺.

Synthesis of compound 148.4. Compound 148.4 was prepared from compound 148.3 following the procedure described in the synthesis of compound 1.12. The product was purified by flash column chromatography on silica gel (2.5% methanol in DCM). MS (ES): m/z 569.6

Synthesis of compound 148.5. Compound 148.5 was prepared from compound 148.4 and following the procedure described in the synthesis of compound 1.14. The product was used in the next step without purification. MS (ES): m/z 469.6 [M+H]⁺.

Synthesis of compound 148.6. Compound 148.6 was prepared from compound 148.5 and 2-chloroethyl carbonochloridate following the procedure described in the synthesis of compound 9.7. The product was purified by flash column chromatography on silica gel (Combiflash®, 2.5% methanol in DCM). MS (ES): m/z 577.05 [M+H]⁺.

Synthesis of compound I-148. Compound I-148 was prepared from compound 148.6 and following the procedure described in the synthesis of compound I-9. The product was purified by flash column chromatography on silica gel (2.4% methanol in DCM). MS (ES): m/z 540.02 [M+H]⁺. ¹H NMR (400 MHz, DMSO-d₆): δ 8.39 (s, 1H), 8.12 (d, J=2.0 Hz, 1H), 7.37 (s, 2H), 7.14 (s, 1H), 6.88 (d, J=7.6 Hz, 1H), 6.79 (s, 1H), 4.58 (d, J=8.4 Hz, 2H), 4.26 (d, J=7.6 Hz, 2H), 3.74 (s, 3H), 3.37 (s, 3H), 3.25 (s, 3H), 2.54 (s, 3H), 2.07-2.00 (m, 2H), 1.73 (t, J=5.6 Hz, 1H), 1.48 (t, J=6.8 Hz, 1H), 1.34 (t, J=7.6 Hz, 1H), 1.23 (d, J=6.4 Hz, 1H).

### Example I-149: 3-((1S,3S)-3-(8-(3,6-dihydro-2H-pyran-4-yl)-9-(2,3-dihydrobenzofuran-6-yl)-3-methyl-2-oxo-2,3-dihydro-1H-imidazo[4,5-d]pyrrolo[1,2-b]pyridazin-1-yl)cyclopentyl)oxazolidin-2-one

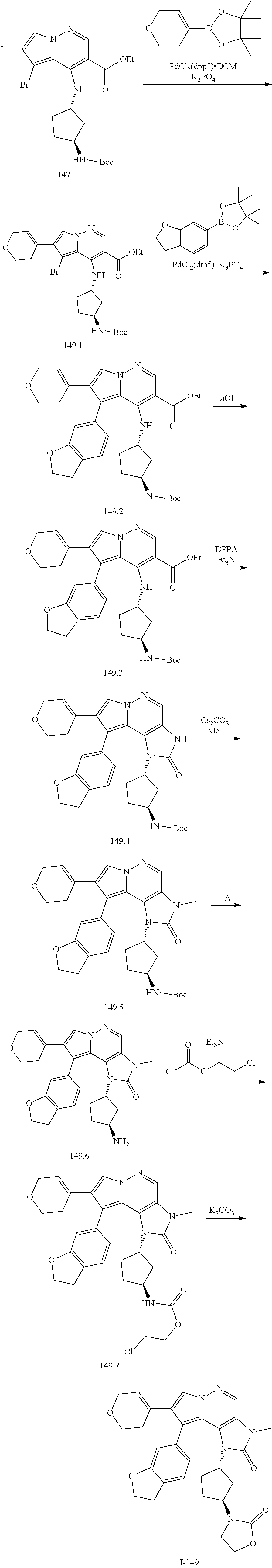

Synthesis of compound 149.1. Compound 149.1 was prepared from compound 147.1 and 2-(3,6-dihydro-2H-pyran-4-yl)-4,4,5,5-tetramethyl-1,3,2-dioxaborolane following the procedure described in the synthesis of compound 54.3. The product was purified by flash column chromatography on silica gel (Combiflash®, 25% ethyl acetate in n-hexane). MS(ES): m/z 549.6 and 551.5 [M+H]⁺.

Synthesis of compound 149.2. Compound 149.2 was prepared from compound 149.1 and 2-(2,3-dihydrobenzofuran-6-yl)-4,4,5,5-tetramethyl-1,3,2-dioxaborolane following the procedure described in the synthesis of compound 59.12. The product was purified by flash column chromatography on silica gel (50% ethyl acetate in n-hexane). MS (ES): m/z 589.7 [M+H]⁺.

Synthesis of compound 149.3. Compound 149.3 was prepared from compound 149.2 following the procedure described in the synthesis of compound 1.10. The product was used in the next step without purification. MS (ES): m/z 561.65 [M+H]⁺.

Synthesis of compound 149.4. Compound 149.4 was prepared from compound 149.3 following the procedure described in the synthesis of compound 1.11. The product was purified by flash column chromatography on silica gel (2.2% methanol in DCM). MS (ES): m/z 558.65 [M+H]⁺.

Synthesis of compound 149.5. Compound 149.5 was prepared from compound 149.4 following the procedure described in the synthesis of compound 1.12. The product was purified by flash column chromatography on silica gel (2.0% methanol in DCM). MS (ES): m/z 572.68 [M+H]⁺.

Synthesis of compound 149.6. Compound 149.6 was prepared from compound 149.5 following the procedure described in the synthesis of compound 1.14. The product was used in the next step without purification. MS (ES): m/z 472.3 [M+H]⁺.

Synthesis of compound 149.7. Compound 149.7 was prepared from compound 149.6 and 2-chloroethyl carbonochloridate following the procedure described in the synthesis of compound 9.7. The product was purified by flash column chromatography on silica gel (Combiflash®, 3.0% methanol in DCM). MS(ES) m/z 579.07 [M+H]⁺.

Synthesis of I-149. Compound I-149 was prepared from compound 149.7 following the procedure described in the synthesis of compound I-9. The product was purified by flash column chromatography on silica gel (Combiflash®, 3.5% methanol in DCM). MS(ES): m/z 542.3. ¹H-NMR (400 MHz, DMSO-d₆): δ 1H-NMR (400 MHz, DMSO-d6): δ 8.41 (s, 1H), 7.92 (s, 1H), 7.41-7.34 (m, 1H), 6.87 (d, J=Hz, 1H), 6.78 (s, 1H), 5.52-5.49 (m, 1H), 4.66-4.55 (m, 3H), 4.35-4.24 (m, 3H), 3.98 (s, 3H), 3.69-3.66 (m, 3H), 3.36 (s, 3H), 3.30-3.25 (m, 2H), 2.34-2.22 (m, 2H), 2.08-1.96 (m, 2H), 1.73-1.72 (m, 1H), 1.49-1.46 (m, 1H), 1.34 (m, 1H), 0.96-0.94 (m, 1H).

### Example I-150: (1R,2R)—N-((1s,4S)-4-(9-(2,3-dihydrobenzo[b][1,4]dioxin-6-yl)-3-methyl-8-(2-methylpyrimidin-4-yl)-2-oxo-2,3-dihydro-1H-imidazo[4,5-d]pyrrolo[1,2-b]pyridazin-1-yl)-1-methylcyclohexyl)-2-fluorocyclopropane-1-carboxamide

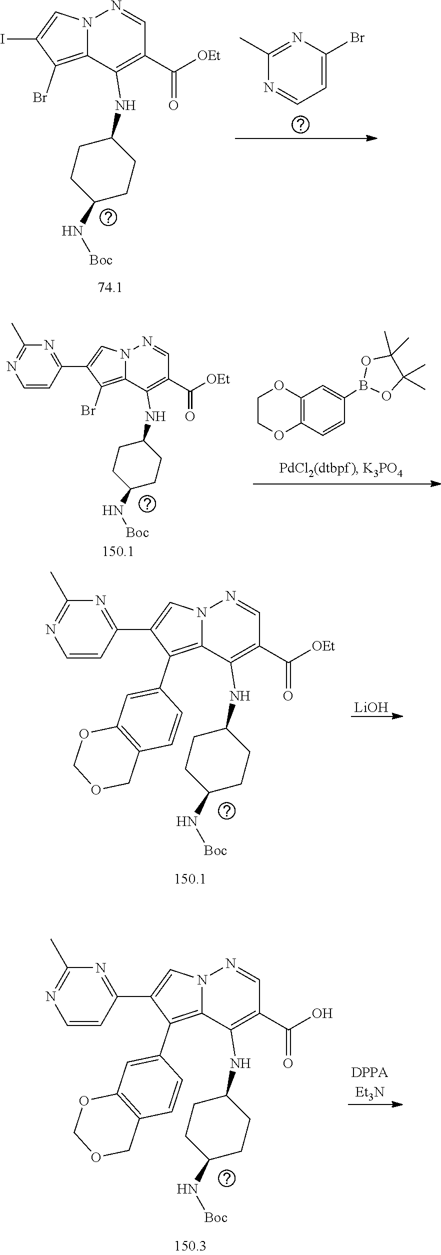

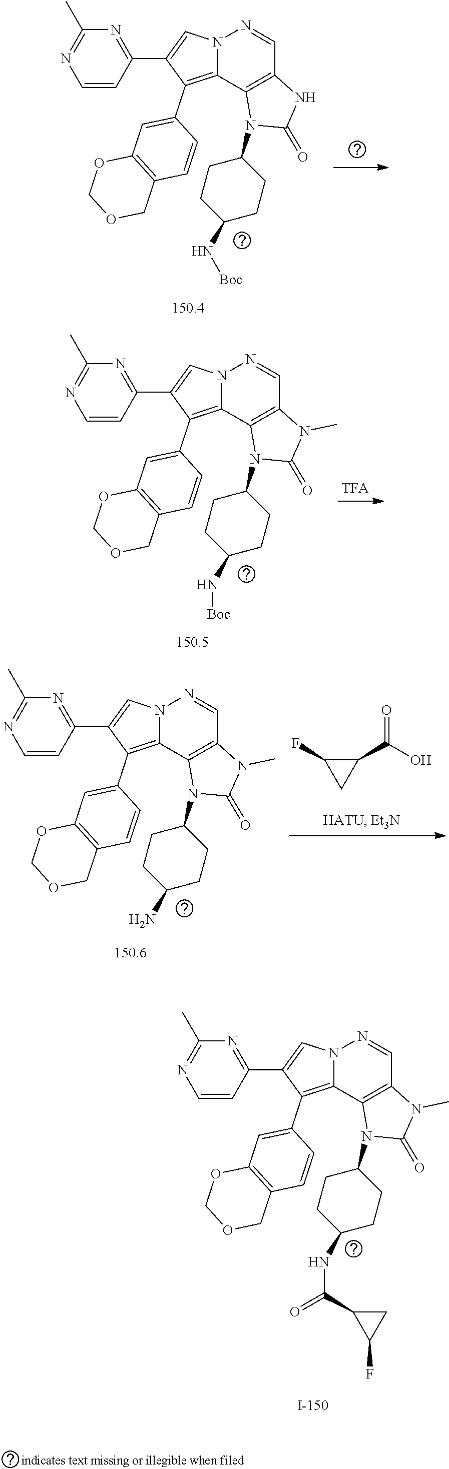

Synthesis of compound 150.1. A mixture of 74.1 (1 g, 5.78 mmol, 1.0 equiv), hexamethylditin (4.6 g, 11.56 mmol, 2.0 equiv) and tetrakis(triphenylphosphine)palladium (0) (0.67 g, 0.578 mmol, 0.1 equiv) in 1,4-dioxane (40 mL) was stirred at 100° C. for 3 h. To the mixture were added 4-bromo-2-methylpyrimidine (0.550 g, 5.85 mmol, 1.0 equiv), tetrakis(triphenylphosphine)palladium (0) (0.102 g, 88.5 mmol, 0.1 equiv), copper(I) iodide (0.336 g, 771 mmol, 2.0 equiv) and lithium chloride (0.083 g, 1771 mmol, 2.0 equiv).

The reaction mixture was stirred at 100° C. for 2 h. It was quenched with ice-water and extract with ethyl acetate. The combined organic layers were washed with brine, dried over anhydrous sodium sulfate, filtered and concentrated under reduced pressure. The residue was purified by flash column chromatography on silica gel (30% ethyl acetate in hexane) to afford 150.1 (0.25 g, 26%). m/z 587.5 and 589.4 [M+H]⁺.

Synthesis of compound 150.2. Compound 150.2 was prepared from compound 150.1 and 2-(2,3-dihydrobenzo[b][1,4]dioxin-6-yl)-4,4,5,5-tetramethyl-1,3,2-dioxaborolane following the procedure described in the synthesis of compound 59.12. The product was purified by flash column chromatography on silica gel (35% ethyl acetate in hexane). MS(ES): m/z 643.8 [M+H]⁺.

Synthesis of compound 150.3. Compound 150.3 was prepared from compound 150.2 following the procedure described in the synthesis of compound 1.10. The product was triturated with pentane, dried under vacuum and used in the next step without purification. MS(ES): m/z 615.7 [M+H]⁺.

Synthesis of compound 150.4. Compound 150.4 was prepared from compound 150.3 following the procedure described in the synthesis of compound 1.11. The product was purified by flash column chromatography on silica gel (Combiflash®, 2.1% methanol in DCM). MS(ES): m/z 612.8 [M+H]⁺.

Synthesis of compound 150.5. Compound 150.5 was prepared from compound 150.4 following the procedure described in the synthesis of compound 1.12. The product was purified by flash column chromatography on silica gel (1.5% methanol in DCM). MS(ES): m/z 626.8 [M+H]⁺.

Synthesis of compound 150.6. Compound 150.6 was prepared from compound 150.5 following the procedure described in the synthesis of compound 1.14. The product was used in the next step without purification. MS(ES): m/z 526.45 [M+H]⁺.

Synthesis of compound I-150. Compound I-150 was prepared from compound 150.6 and (1R,2R)-2-fluorocyclopropane-1-carboxylic acid following the procedure described in the synthesis of compound I-45. The product was purified by flash column chromatography on silica gel (3.1% methanol in DCM). MS(ES): m/z 612.3 [M+H]⁺, ¹H-NMR (400 MHz, DMSO-d₆): δ 8.57 (s, 1H), 8.49 (s, 1H), 8.41 (d, J=5.2 Hz, 1H), 7.47 (s, 1H), 7.05 (d, J=11.2 Hz, 1H), 6.92-6.87 (m, 2H), 6.53 (d, J=5.2 Hz, 1H), 4.68 (m, 0.5H), 4.62 (m, 0.5H), 4.36-4.30 (m, 4H), 3.36 (s, 3H), 2.56 (s, 3H), 2.45 (m, 2H), 2.21-2.03 (m, 4H), 1.92-1.83 (m, 1H), 1.50-1.42 (m, 1H), 1.13 (m, 4H), 1.00-0.98 (m, 1H), 0.56-0.53 (m, 2H).

### Example I-151 (1S,3S)-3-(9-(benzo[d][1,3]dioxol-5-yl)-8-(3-methoxy-1-methyl-1H-pyrazol-4-yl)-3-methyl-2-oxo-2,3-dihydro-1H-imidazo[4,5-d]pyrrolo[1,2-b]pyridazin-1-yl)-N-(tetrahydro-2H-pyran-4-yl)cyclopentane-1-carboxamide

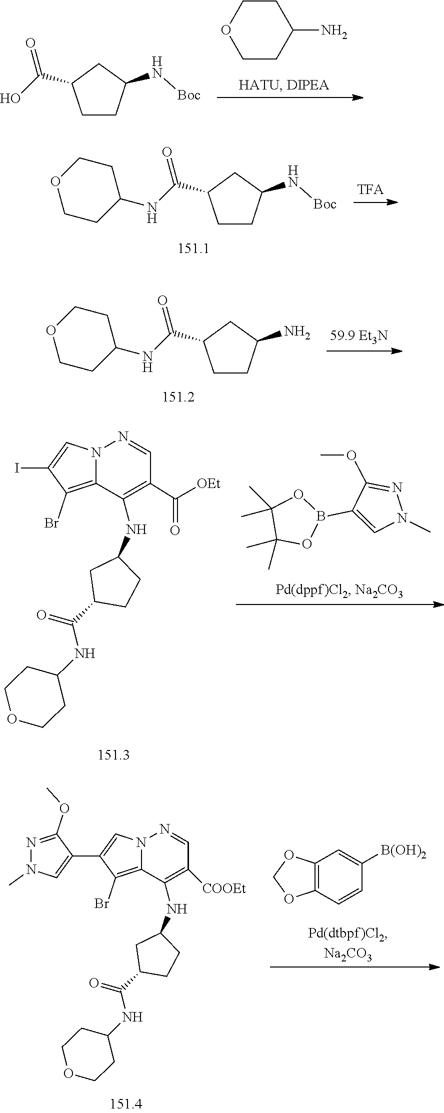

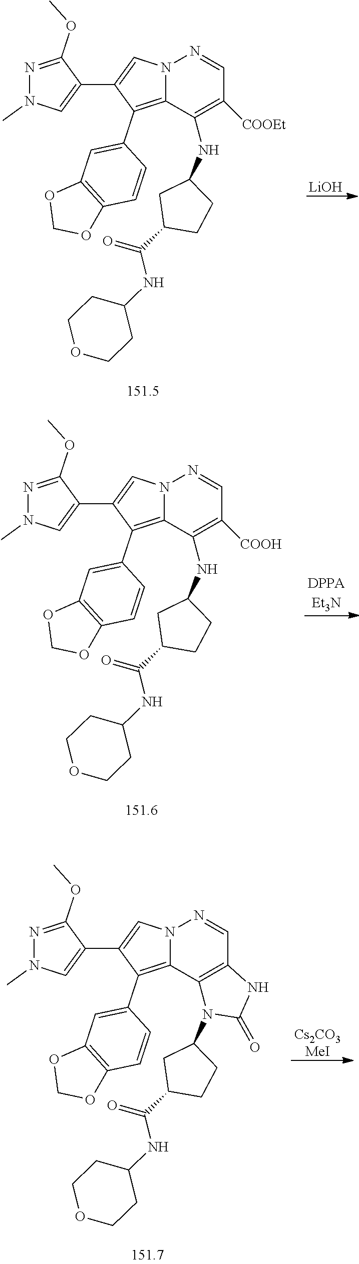

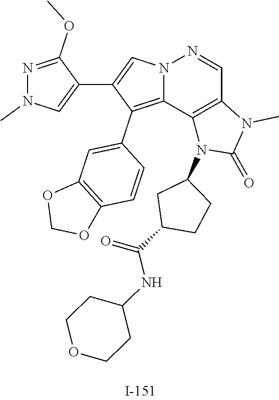

Synthesis of compound 151.1 To a solution of (1r,4r)-4-((tert-butoxycarbonyl)amino)cyclohexane-1-carboxylic acid (5.0 g, 2.18 mmol, 1.0 equiv) in DMF (5 mL) was added HATU (12.4 g, 3.27 mmol, 1.5 equiv) at 0° C. and stirred for 30 min. To this reaction mixture N,N-diisopropylethylamine (8.436 g, 6.54 mmol, 3.0 equiv) and (S)-tetrahydrofuran-3-amine (2.64 g, 2.61 mmol, 1.2 equiv) were added at 0° C. It transferred into water and extracted with ethyl acetate, washed with brine, dried over anhydrous sodium sulfate, filtered and concentrated under reduced pressure to afford 151.1 (2.0 g, 29%). MS(ES): m/z 313.4 [M+H]⁺.

Synthesis of compound 151.2. To a solution of 151.1 (2.0 g, 0.638 mmol, 1.0 equiv) in DCM (20 mL) was added trifluoroacetic acid (10 mL) dropwise at 0° C. The reaction mixture was stirred at room temperature for 1 h. It was neutralized by sodium bicarbonate solution and extracted with DCM. The combined organic layers were dried over anhydrous sodium sulfate, filtered and dried under reduced pressure to afford 151.2 (1.5 g). MS(ES): m/z 213.4 [M+H]⁺.

Synthesis of compound 151.3. Compound 151.3 was prepared from compound 151.2 and 59.9 following the procedure described in the synthesis of compound 1.9. The product was purified by flash column chromatography on silica gel (35% ethyl acetate in hexane). MS(ES): m/z 605.2 and 607.2 [M+H]⁺.

Synthesis of compound 151.4. Compound 151.4 was prepared from compound 151.3 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (55% ethyl acetate in hexane). MS(ES): m/z 589.4 and 590.4 [M+H]⁺.

Synthesis of compound 151.5. Compound 151.5 was prepared from compound 151.4 and benzo[d][1,3]dioxol-5-ylboronic acid following the procedure described in the synthesis of compound 59.12. The product was purified by flash column chromatography on silica gel (55% ethyl acetate in hexane). MS(ES): m/z 631.3 [M+H]⁺.

Synthesis of compound 151.6. Compound 151.6 was prepared from compound 151.5 following the procedure described in the synthesis of compound 1.10. The product was triturated with pentane, dried under vacuum and used in the next step without purification. MS(ES): m/z 603.6 [M+H]⁺.

Synthesis of compound 151.7. Compound 151.7 was prepared from compound 151.6 following the procedure described in the synthesis of compound 1.11. The product was purified by flash column chromatography on silica gel (Combiflash®, 2.1% methanol in DCM). MS(ES): m/z 600.6 [M+H]⁺.

Synthesis of compound I-151. Compound I-151 was prepared from compound 151.7 following the procedure described in the synthesis of compound 1.12. The product was purified by flash column chromatography on silica gel (2.3% methanol in DCM). MS (ES): m/z 614.3 [M+H]⁺. ¹H-NMR (400 MHz, DMSO-d₆): δ 8.29 (s, 1H), 8.00 (d, J=2.4 Hz, 1H), 7.53 (s, 1H), 6.97 (d, J=8.0 Hz, 1H), 6.84 (d, J=4.8 Hz, 1H), 6.81 (s, 1H), 6.54 (s, 1H), 6.15 (s, 2H), 3.94 (s, 3H), 3.90 (d, J=2.4 Hz, 3H), 3.74-3.83 (m, 2H), 3.68 (s, 3H), 3.42 (s, 3H), 3.32-3.37 (m, 2H), 2.94 (m, 2H), 2.05 (t, J=6.8 Hz, 2H), 1.81 (m, 1H), 1.70 (m, 2H), 1.42 (m, 2H), 1.12 (d, J=4.4 Hz, 1H).

### Example I-152: (1r,4S)-4-(9-(benzo[d][1,3]dioxol-5-yl)-3-methyl-8-(1-methyl-1H-pyrazol-4-yl)-2-oxo-2,3-dihydro-1H-imidazo[4,5-d]pyrrolo[1,2-b]pyridazin-1-yl)-N—((S)-tetrahydrofuran-3-yl)cyclohexane-1-carboxamide

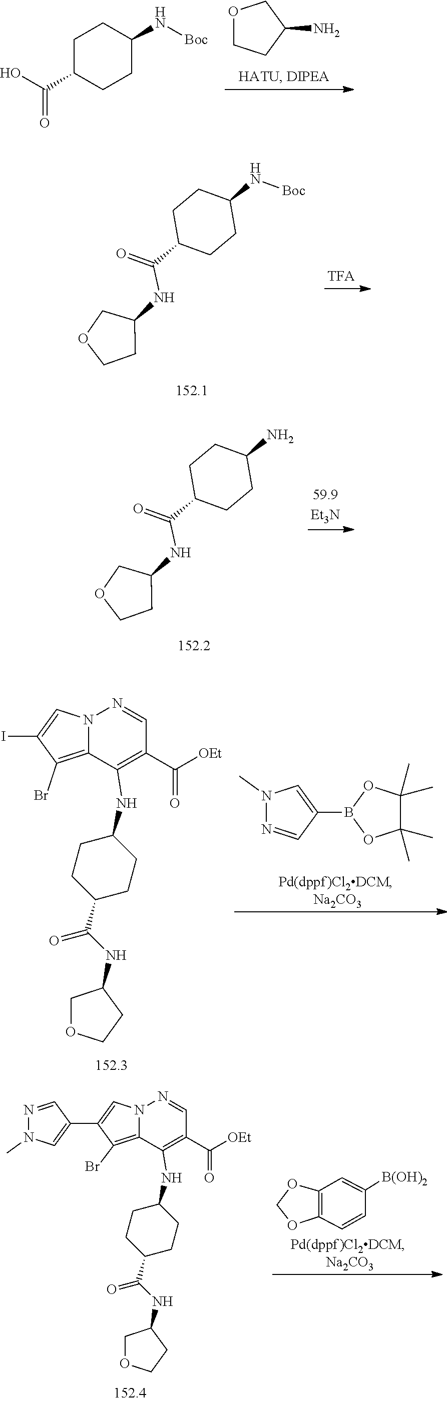

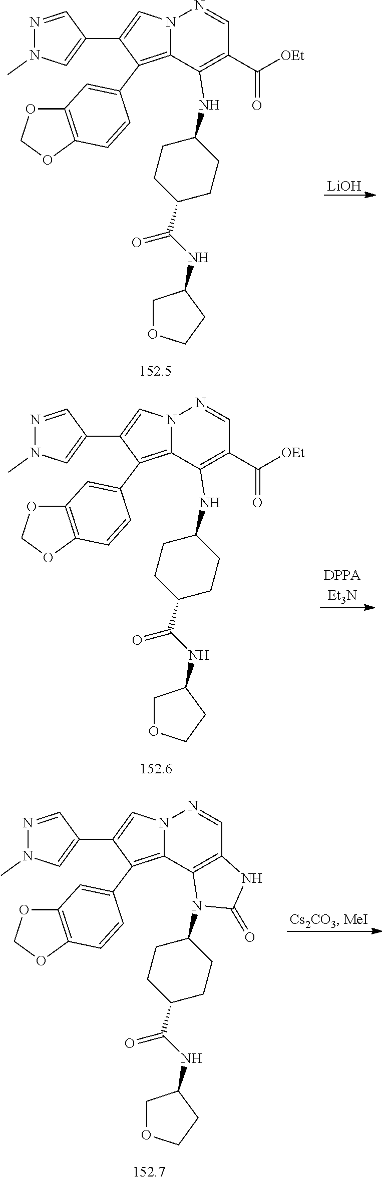

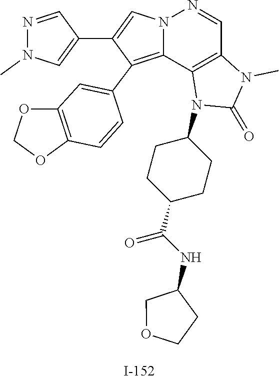

Synthesis of compound 152.1. To a solution of (1r,4r)-4-((tert-butoxycarbonyl)amino)cyclohexane-1-carboxylic acid (2 g, 8.22 mmol, 1.0 equiv) in DMF (40 mL) was added HATU (4.68 g, 12.33 mmol, 1.5 equiv) at 0° C. The reaction mixture was stirred for another 30 min. To the mixture was added (S)-tetrahydrofuran-3-amine (0.93 g, 10.69 mmol, 1.3 equiv) and N,N-diisopropylethylamine (3.2 mL, 24.66 mmol, 3.0 equiv) at 0° C. and stirred at room temperature for 30 min. It was quenched with ice-water and extract with ethyl acetate. The combined organic layers were washed with brine, dried over anhydrous sodium sulfate, filtered and concentrated under reduced pressure. The residue was purified by flash column chromatography on silica gel (30% ethyl acetate in hexane) to afford 152.1 (0.99 g, 43%). MS (ES): m/z 313.5 [M+H]⁺.

Synthesis of compound 152.2. To a solution of 152.1 (1.1 g, 3.52 mmol, 1.0 equiv) in DCM (20 mL) was added dropwise trifluoroacetic acid (3 mL) at 0° C. The reaction mixture was stirred at room temperature for 1 h. It was concentrated under reduced pressure to afford 152.2 (0.99 g). MS (ES): m/z 213.3 [M+H]⁺.

Synthesis of compound 152.3. Compound 152.3 was prepared from compound 152.2 and 59.9 following the procedure described in the synthesis of compound 1.9. The product was purified by flash column chromatography on silica gel (25% ethyl acetate in hexane). MS (ES): m/z 606.3 [M+H]⁺.

Synthesis of compound 152.4. Compound 152.4 was prepared from compound 152.3 and 1-methyl-4-(4,4,5,5-tetramethyl-1,3,2-dioxaborolan-2-yl)-1H-pyrazole following the procedure described in the synthesis of compound 1.13. The product was purified by flash column chromatography on silica gel (35% ethyl acetate in hexane). MS(ES): m/z 560.5 [M+H]⁺.

Synthesis of compound 152.5. Compound 152.5 was prepared from compound 152.4 and benzo[d][1,3]dioxol-5-ylboronic acid following the procedure described in the synthesis of compound 1.13. The product was purified by flash column chromatography on silica gel (47% ethyl acetate in hexane). MS(ES): m/z 601.8 [M+H]⁺.

Synthesis of compound 152.6. Compound 152.6 was prepared from compound 152.5 following the procedure described in the synthesis of compound 1.10. The product was triturated with pentane, dried under vacuum and used in the next step without purification. MS(ES): m/z 573.7 [M+H]⁺.

Synthesis of compound 152.7. Compound 152.7 was prepared from compound 152.6 following the procedure described in the synthesis of compound 1.11. The product was purified by flash column chromatography on silica gel (Combiflash®, 1.8% methanol in DCM). MS(ES): m/z 570.6 [M+H]⁺.

Synthesis of compound I-152. Compound I-152 was prepared from compound 152.7 following the procedure described in the synthesis of compound 1.12. The product was purified by flash column chromatography on silica gel (3.1% methanol in DCM). MS(ES): m/z 584.32 [M+H]⁺. ¹H-NMR (400 MHz, DMSO-d₆): δ 8.37 (s, 1H), 8.11 (s, 1H), 7.94 (d, J=6.4 Hz, 1H), 7.25 (s, 1H), 7.09 (s, 1H), 7.01 (d, J=1.2 Hz, 1H), 6.94 (s, 1H), 6.78 (dd, J=1.60, 8.0 Hz, 1H), 6.26 (d, J=7.2 Hz, 2H), 4.19 (bs, 1H), 3.79-3.68 (m, 5H), 3.67-3.65 (m, 1H), 3.51-3.45 (m, 1H), 3.40-3.38 (m, 1H), 3.35 (s, 3H), 2.36-2.31 (m, 1H), 2.20-2.17 (m, 1H), 2.08-2.03 (m, 1H), 1.98-1.92 (m, 1H), 1.69-1.65 (m, 1H), 1.58-1.38 (m, 3H), 1.36-1.24 (m, 1H), 1.02-0.97 (m, 1H), 0.76-0.70 (m, 1H).

### Example I-153: 3-((1S,3S)-3-(8-(2,4-dimethyloxazol-5-yl)-3-methyl-9-(1-methyl-1H-indazol-5-yl)-2-oxo-2,3-dihydro-1H-imidazo[4,5-d]pyrrolo[1,2-b]pyridazin-1-yl)cyclopentyl)oxazolidin-2-one

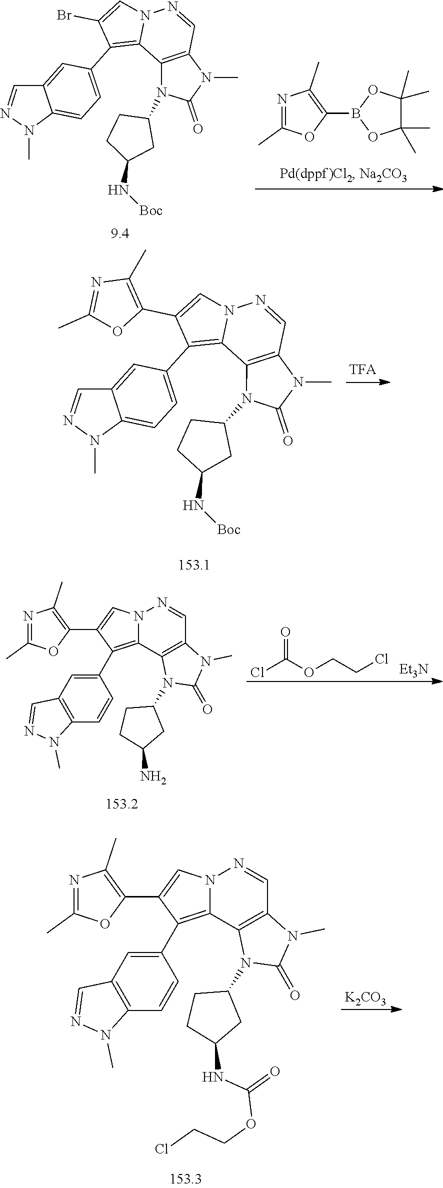

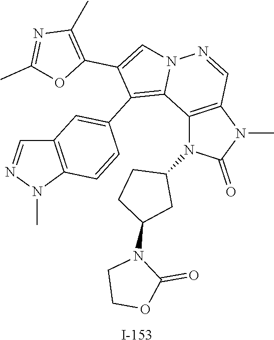

Synthesis of compound 153.1. Compound 153.1 was prepared from compound 9.4 and 2,4-dimethyl-5-(4,4,5,5-tetramethyl-1,3,2-dioxaborolan-2-yl)oxazole following the procedure described in the synthesis of compound 1.13. The product was purified by flash column chromatography on silica gel (2.4% methanol in DCM). MS(ES): m/z 597.4 [M+H]⁺.

Synthesis of compound 153.2. Compound 153.2 was prepared from compound 153.1 following the procedure described in the synthesis of compound 1.14. MS(ES): m/z 497.3 [M+H]⁺.

Synthesis of compound 153.3. Compound 153.3 was prepared from compound 153.2 and 2-chloroethyl carbonochloridate following the procedure described in the synthesis of compound 9.7. The product was purified by flash column chromatography on silica gel (Combiflash®, 3.6% methanol in DCM). MS(ES): m/z 604.2 [M+H]⁺.

Synthesis of I-153. Compound I-153 was prepared from compound 153.3 following the procedure described in the synthesis of compound I-9. The product was purified by flash column chromatography on silica gel (Combiflash®, 4.0% methanol in DCM). MS(ES): m/z 567.34 [M+H]⁺. ¹H-NMR (400 MHz, DMSO-d₆): δ 8.53 (s, 1H), 8.15-8.09 (m, 2H), 7.81-7.73 (m, 2H), 7.45-7.38 (m, 1H), 4.20-4.04 (m, 6H), 3.56 (s, 1H), 3.39 (s, 3H), 3.00-2.81 (m, 1H), 2.56 (s, 1H), 2.27-2.11 (m, 4H), 2.01-1.98 (m, 1H), 1.87 (s, 3H), 1.59 (s, 1H), 1.43 (s, 1H), 1.25 (s, 1H), 0.88 (s, 0.5H), 0.44 (s, 0.5H).

### Example I-154: 3-((1S,3S)-3-(9-(6,7-dihydro-4H-thieno[3,2-c]pyran-2-yl)-3-methyl-2-oxo-8-(thiazol-2-yl)-2,3-dihydro-1H-imidazo[4,5-d]pyrrolo[1,2-b]pyridazin-1-yl)cyclopentyl)oxazolidin-2-one

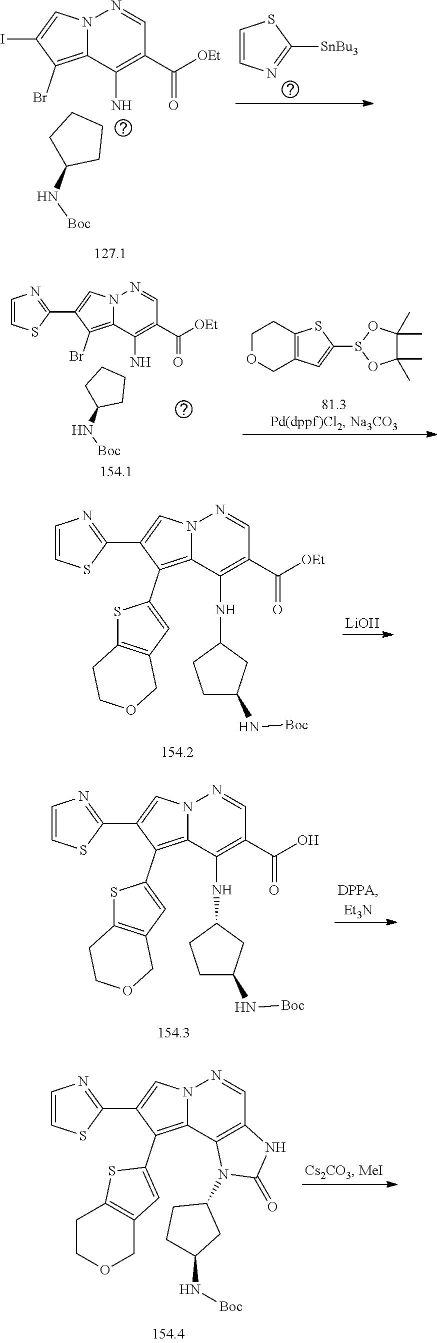

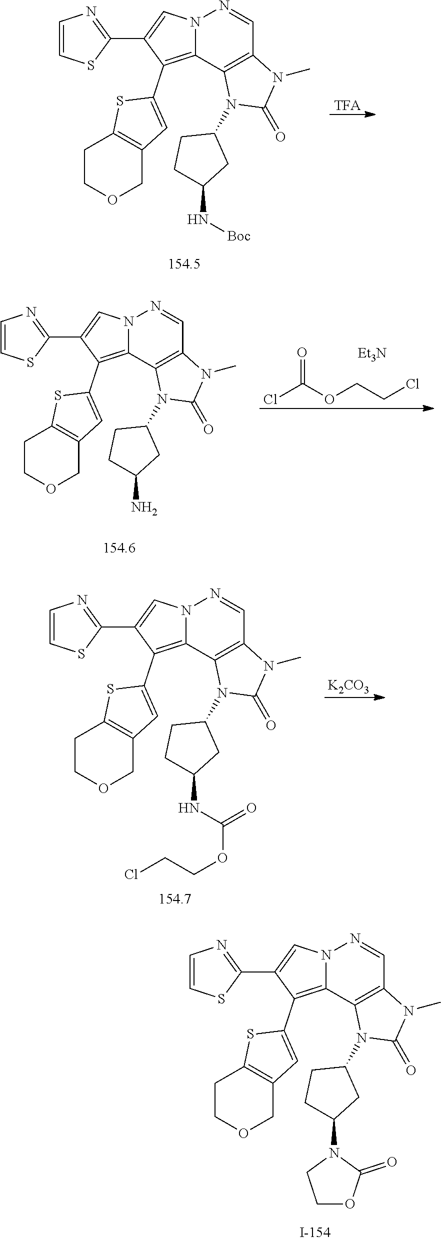

Synthesis of compound 154.1. Compound 154.1 was prepared from compound 127.1 and 2-(tributylstannyl)thiazole following the procedure described in the synthesis of compound I-116. The product was purified by flash column chromatography on silica gel (Combiflash®, 51% ethyl acetate in hexane). MS(ES): m/z 550.2 and 552.2 [M+H]⁺.

Synthesis of compound 154.2. Compound 154.2 was prepared from compound 154.1 and 81.3 following the procedure described in the synthesis of compound 1.13. The product was purified by flash column chromatography on silica gel (55% ethyl acetate in hexane). MS(ES): m/z 610.54 [M+H]⁺.

Synthesis of compound 154.3. Compound 154.3 was prepared from compound 154.2 following the procedure described in the synthesis of compound 1.10. The product was triturated with pentane, dried under vacuum and used in the next step without purification. MS(ES): m/z 582.5 [M+H]⁺.

Synthesis of compound 154.4. Compound 154.4 was prepared from compound 154.3 following the procedure described in the synthesis of compound 1.11. The product was purified by flash column chromatography on silica gel (Combiflash®, 2.1% methanol in DCM) to afford 1.6 (0.11 g, 61.43%). MS(ES): m/z 579.6 [M+H]⁺.

Synthesis of compound 154.5. Compound 154.5 was prepared from compound 154.4 following the procedure described in the synthesis of compound 1.12. The product was purified by flash column chromatography on silica gel (1.8% methanol in DCM). MS(ES): m/z 593.73 [M+H]⁺.

Synthesis of compound 154.6. Compound 154.6 was prepared from compound 154.5 following the procedure described in the synthesis of compound 1.14. The product was used in the next step without purification. MS(ES): m/z 493.62 [M+H]⁺.

Synthesis of compound 154.7. Compound 154.7 was prepared from compound 154.6 and 2-chloroethyl carbonochloridate following the procedure described in the synthesis of compound 9.7. The product was purified by flash column chromatography on silica gel (Combiflash®, 3.5% methanol in DCM). MS(ES): m/z 600.1 [M+H]⁺.

Synthesis of I-154. Compound I-154 was prepared from compound 154.7 following the procedure described in the synthesis of compound I-9. The product was purified by flash column chromatography on silica gel (Combiflash®, 3.2% methanol in DCM). MS(ES) 563.2 [M+H]⁺. ¹H-NMR (400 MHz, DMSO-d₆): δ 8.94 (s, 1H), 8.55 (s, 1H), 8.46 (s, 1H), 8.03 (s, 1H), 6.99 (d, J=15.6 Hz, 1H), 4.68 (s, 2H), 4.43 (s, 1H), 4.27-4.26 (m, 2H), 3.96-3.94 (m, 3H), 3.39 (s, 4H), 2.90 (s, 2H), 2.54 (s, 2H), 2.17-2.13 (m, 2H), 1.82 (m, 1H), 1.62-1.59 (m, 1H), 1.56-1.51 (m, 1H).

### Example I-155: 3-((1S,3S)-3-(9-(4,7-dihydro-5H-thieno[2,3-c]pyran-2-yl)-8-(3-methoxy-1-methyl-1H-pyrazol-4-yl)-3-methyl-2-oxo-2,3-dihydro-1H-imidazo[4,5-d]pyrrolo[1,2-b]pyridazin-1-yl)cyclopentyl)oxazolidin-2-one

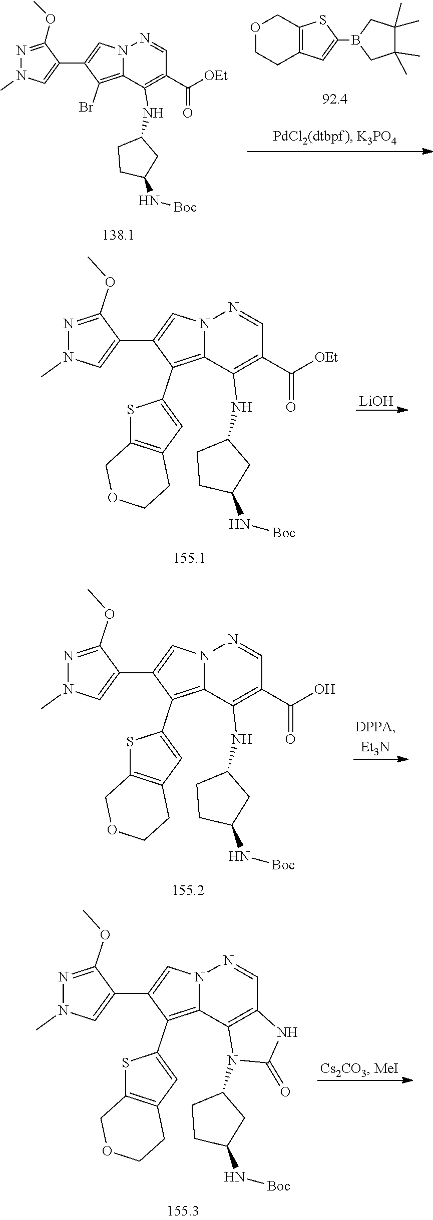

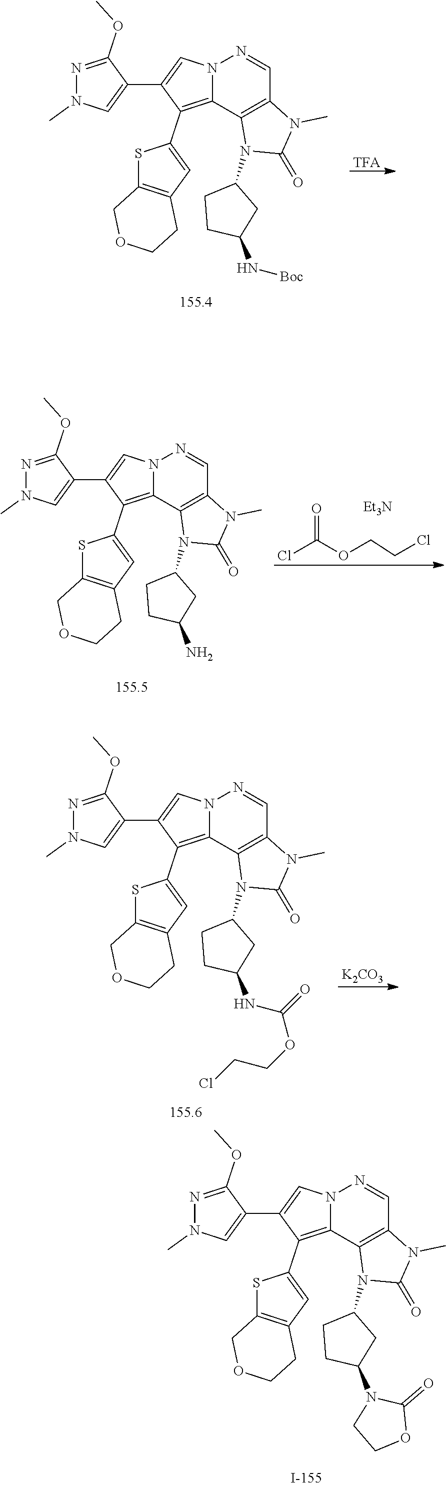

Synthesis of compound 155.1. Compound 155.1 was prepared from compound 138.1 and 92.4 following the procedure described in the synthesis of compound 59.12. The product was purified by flash column chromatography on silica gel (1.4% methanol in DCM). MS(ES): m/z 637.6 [M+H]⁺.

Synthesis of compound 155.2. Compound 155.2 was prepared from compound 155.1 following the procedure described in the synthesis of compound 1.10. The product was triturated with pentane, dried under vacuum and used in the next step without purification. MS(ES): m/z 609.5 [M+H]⁺.

Synthesis of compound 155.3. Compound 155.3 was prepared from compound 155.2 following the procedure described in the synthesis of compound 1.11. The product was purified by flash column chromatography on silica gel (Combiflash®, 1.9% methanol in DCM). MS(ES): m/z 606.4 [M+H]⁺.

Synthesis of compound 155.4. Compound 155.4 was prepared from compound 155.3 following the procedure described in the synthesis of compound 1.12. The product was purified by flash column chromatography on silica gel (1.3% methanol in DCM). MS(ES): m/z 620.8 [M+H]⁺.

Synthesis of compound 155.5. Compound 155.5 was prepared from compound 155.4 following the procedure described in the synthesis of compound 1.14. The product was used in the next step without purification. MS(ES) m/z 520.32 [M+H]⁺.

Synthesis of compound 155.6. Compound 155.6 was prepared from compound 155.5 and 2-chloroethyl carbonochloridate following the procedure described in the synthesis of compound 9.7. The product was purified by flash column chromatography on silica gel (Combiflash®, 3.5% methanol in DCM). MS(ES): m/z 627.16 [M+H]⁺.

Synthesis of I-155. Compound I-155 was prepared from compound 155.6 following the procedure described in the synthesis of compound I-9. The product was purified by flash column chromatography on silica gel (Combiflash®, 3.9% methanol in DCM). MS(ES) m/z: m/z 590.2 [M+H]⁺. ¹H-NMR (400 MHz, DMSO-d₆): δ 8.45 (s, 1H), 8.03 (s, 1H), 6.97 (s, 1H), 6.72 (s, 1H), 4.81 (s, 2H), 4.43-4.41 (m, 1H), 4.29-4.24 (m, 2H), 3.96-3.71 (m, 3H), 3.62 (s, 3H), 3.43 (m, 2H), 3.34 (s, 3H), 2.78 (m, 1H), 2.58 (m, 1H), 2.51 (s, 2H), 2.12-2.09 (m, 2H), 1.82 (m, 1H), 1.61 (m, 2H), 1.36 (m, 2H).

### Example I-156: 3-((1S,3S)-3-(8-(3,6-dihydro-2H-pyran-4-yl)-9-(4,7-dihydro-5H-thieno[2,3-c]pyran-2-yl)-3-methyl-2-oxo-2,3-dihydro-1H-imidazo[4,5-d]pyrrolo[1,2-b]pyridazin-1-yl)cyclopentyl)oxazolidin-2-one

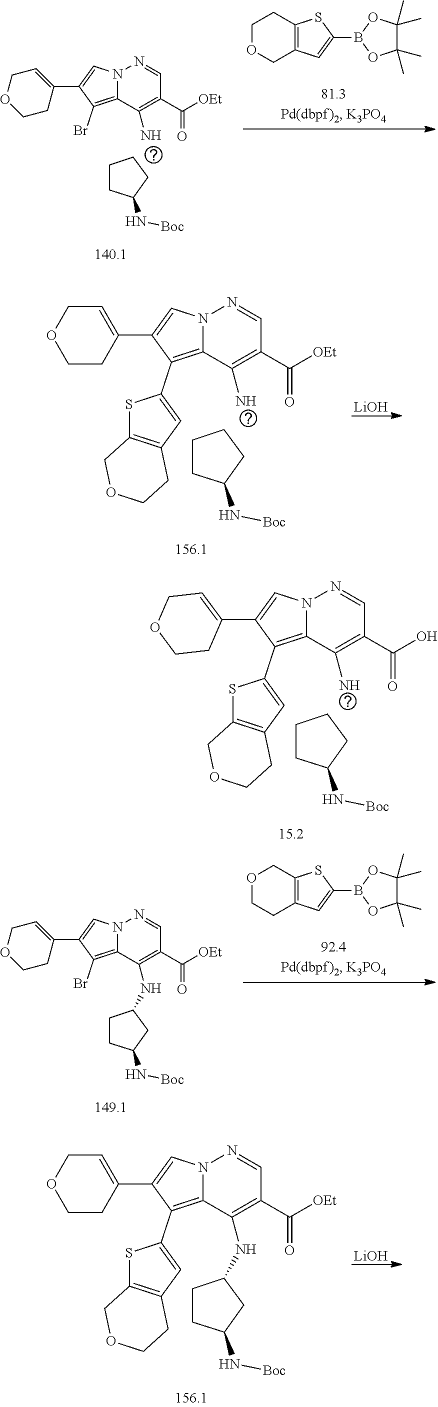

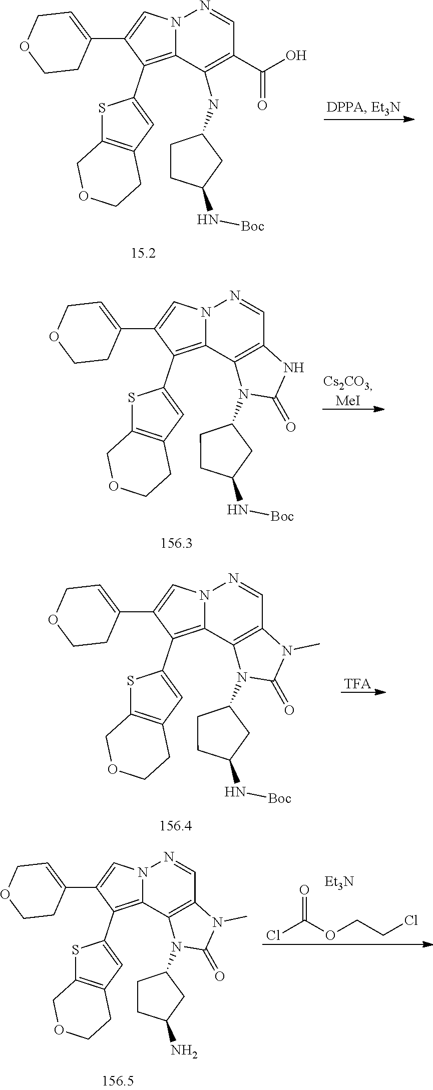

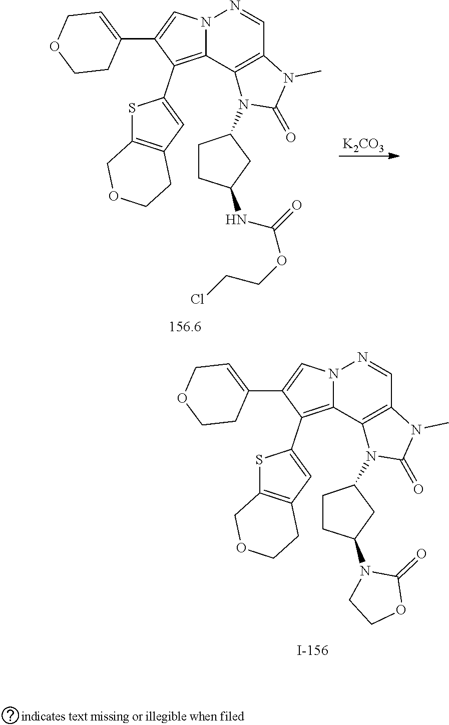

Synthesis of compound 156.1. Compound 156.1 was prepared from compound 140.1 and 92.4 following the procedure described in the synthesis of compound 59.12. The product was purified by flash column chromatography on silica gel (1.7% methanol in DCM). MS(ES): m/z 609.6 [M+H]⁺.

Synthesis of compound 156.2. Compound 156.2 was prepared from compound 156.1 following the procedure described in the synthesis of compound 1.10. The product was triturated with pentane, dried under vacuum and used in the next step without purification. MS(ES): m/z 581.4 [M+H]⁺.

Synthesis of compound 156.3. Compound 156.3 was prepared from compound 156.2 following the procedure described in the synthesis of compound 1.11. The product was purified by flash column chromatography on silica gel (Combiflash®, 2.1% methanol in DCM). MS(ES): m/z 578.9 [M+H]⁺.

Synthesis of compound 156.4. Compound 156.4 was prepared from compound 156.3 following the procedure described in the synthesis of compound 1.12. The product was purified by flash column chromatography on silica gel (1.5% methanol in DCM). MS(ES): m/z 592.4 [M+H]⁺.

Synthesis of compound 156.5. Compound 156.5 was prepared from compound 156.4 following the procedure described in the synthesis of compound 1.14. The product was used in the next step without purification. MS(ES) m/z 492.8 [M+H]⁺.

Synthesis of compound 156.6. Compound 156.6 was prepared from compound 156.5 and 2-chloroethyl carbonochloridate following the procedure described in the synthesis of compound 9.7. The product was purified by flash column chromatography on silica gel (Combiflash®, 3.7% methanol in DCM). MS(ES): m/z 599.6 [M+H]⁺.

Synthesis of I-156. Compound I-156 was prepared from compound 156.6 following the procedure described in the synthesis of compound I-9. The product was purified by flash column chromatography on silica gel (Combiflash®, 4.0% methanol in DCM). MS(ES): m/z 562.43 [M+H]⁺. ¹H-NMR (400 MHz, DMSO-d₆): δ 8.47 (s, 1H), 7.95 (s, 1H), 6.95 (s, 1H), 5.70 (s, 1H), 4.73-4.82 (m, 2H), 4.42-4.47 (m, 1H), 4.29-4.24 (m, 2H), 4.06 (m, 2H), 3.96-3.93 (m, 2H), 3.73 (t, J=5.6 Hz, 2H), 3.45-3.41 (m, 2H), 3.39 (m, 4H), 2.73 (m, 2H), 2.30 (m, 2H), 2.08-2.06 (m, 2H), 1.83-1.80 (m, 1H), 1.66 (m, 1H), 1.56-1.54 (m, 1H), 1.25-1.21 (m, 1H).

### Example I-157: 3-((1S,3S)-3-(3-methyl-9-(1-methyl-1H-indazol-5-yl)-2-oxo-8-(thiazol-2-yl)-2,3-dihydro-1H-imidazo[4,5-d]pyrrolo[1,2-b]pyridazin-1-yl)cyclopentyl)oxazolidin-2-one

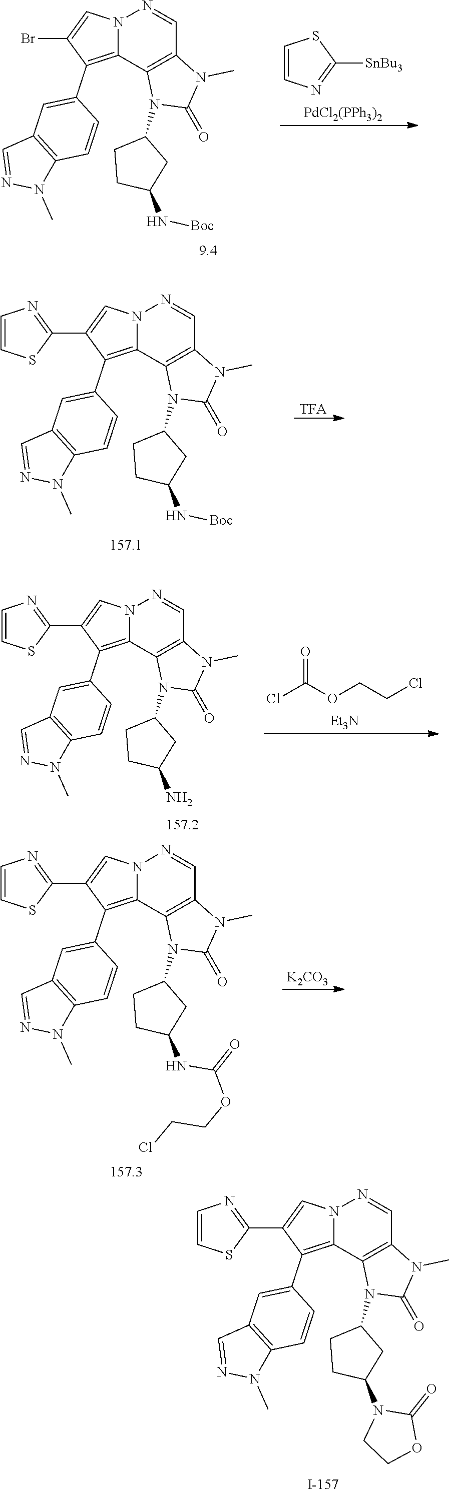

Synthesis of compound 157.1. A mixture of 9.4 (0.250 g, 0.43 mmol, 1.0 equiv) and 2-(tributylstannyl)thiazole (0.483 g, 1.29 mmol, 3.0 equiv) in 1,4-dioxane (6 mL) was degassed by bubbling through a stream of argon for 10 min. Bis(triphenylphosphine)palladium (II) chloride (0.065 g, 0.09 mmol, 0.2 equiv) was added and degassed for 5 min. The reaction mixture was stirred at 150° C. in a microwave reactor for 8 h. It was cooled to room temperature, filtered through a pad of Celite® and rinsed with ethyl acetate. The filtrate was dried over anhydrous sodium sulfate, filtered and concentrated under reduced pressure. The residue was purified by flash column chromatography on silica gel (3.0% methanol in DCM) to afford 157.1 (0.105 g, 42%). MS(ES): m/z 585.40 [M+H]⁺.

Synthesis of compound 157.2. Compound 157.2 was prepared from compound 157.1 following the procedure described in the synthesis of compound 1.14. The product was used in the next step without purification. MS(ES): m/z 485.30 [M+H]⁺.

Synthesis of compound 157.3. Compound 157.3 was prepared from compound 157.2 and 2-chloroethyl carbonochloridate following the procedure described in the synthesis of compound 9.7. The product was purified by flash column chromatography on silica gel (Combiflash®, 3.1% methanol in DCM). MS(ES): m/z 592.3 [M+H]⁺.

Synthesis of I-157. Compound I-157 was prepared from compound 157.3 following the procedure described in the synthesis of compound I-9. The product was purified by flash column chromatography on silica gel (Combiflash®, 3.2% methanol in DCM). MS(ES): m/z 555.5 [M+H]⁺. ¹H-NMR (400 MHz, DMSO-d₆): δ 8.75 (s, 1H), 8.52 (s, 1H), 8.48 (s, 1H), 8.14 (d, J=6.4 Hz, 1H), 7.93-7.84 (m, 3H), 7.47-7.40 (m, 1H), 4.26-4.21 (m, 1H), 4.14 (s, 3H), 4.12 (m, 1H), 4.07-4.05 (m, 1H), 3.56-3.52 (m, 1H), 3.33 (s, 3H), 3.07-3.02 (m, 1H), 2.86-2.80 (m, 1H), 2.28-2.23 (m, 1H), 2.04-1.82 (m, 3H), 1.38-1.36 (m, 2H).

### Example I-158: 3-((1S,3S)-3-(9-(2,3-dihydrobenzofuran-6-yl)-3-methyl-8-(2-methylthiazol-5-yl)-2-oxo-2,3-dihydro-1H-imidazo[4,5-d]pyrrolo[1,2-b]pyridazin-1-yl)cyclopentyl)oxazolidin-2-one

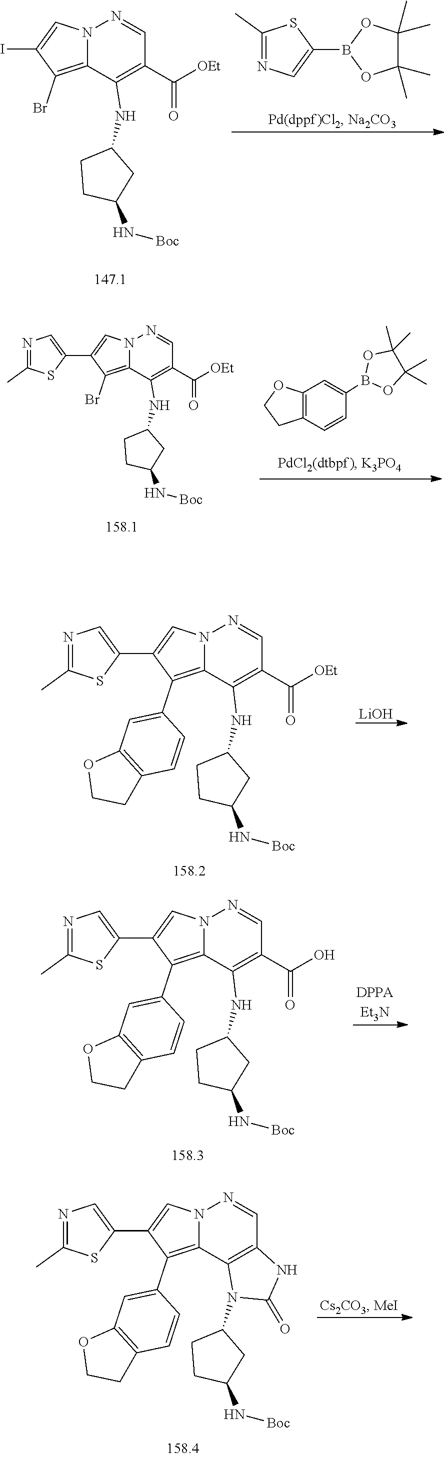

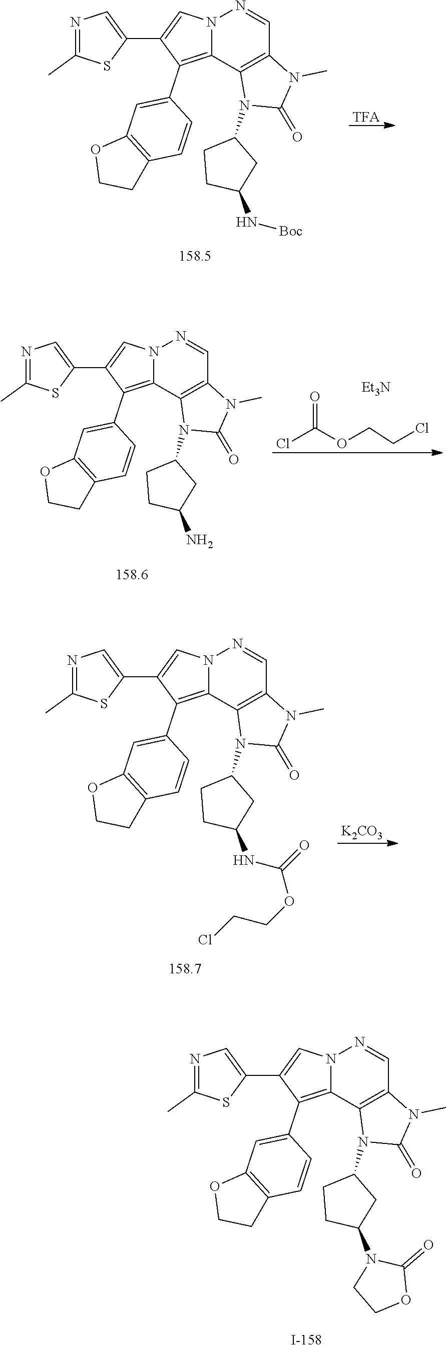

Synthesis of compound 158.1. Compound 158.1 was prepared from compound 147.1 and 2-methyl-5-(4,4,5,5-tetramethyl-1,3,2-dioxaborolan-2-yl)thiazole following the procedure described in the synthesis of compound 1.13. The product was purified by flash column chromatography on silica gel (48% ethyl acetate in n-hexane). MS (ES): m/z 564.4 and 566.5 [M+H]⁺.

Synthesis of compound 158.2. Compound 158.2 was prepared from compound 158.1 and 2-(2,3-dihydrobenzofuran-6-yl)-4,4,5,5-tetramethyl-1,3,2-dioxaborolane following the procedure described in the synthesis of compound 59.12. The product was purified by flash column chromatography on silica gel (40% ethyl acetate in n-hexane). MS (ES): m/z 604.8 [M+H]⁺.

Synthesis of compound 158.3. Compound 158.3 was prepared from compound 158.2 following the procedure described in the synthesis of compound 1.10. The product was used in the next step without purification. MS (ES): m/z 576.5 [M+H]⁺.

Synthesis of compound 158.4. Compound 158.4 was prepared from compound 158.3 following the procedure described in the synthesis of compound 1.11. The product was purified by flash column chromatography on silica gel (2.5% methanol in DCM). MS (ES): m/z 573.8 [M+H]⁺.

Synthesis of compound 158.5. Compound 158.5 was prepared from compound 158.4 following the procedure described in the synthesis of compound 1.12. The product was purified by flash column chromatography on silica gel (2.5% methanol in DCM). MS (ES): m/z 587.6 [M+H]⁺.

Synthesis of compound 158.6. Compound 158.6 was prepared from compound 158.5 following the procedure described in the synthesis of compound 1.14. The product was used in the next step without purification. MS (ES): m/z 487.5 [M+H]⁺.

Synthesis of compound 158.7. Compound 158.7 was prepared from compound 158.6 and 2-chloroethyl carbonochloridate following the procedure described in the synthesis of compound 9.7. The product was purified by flash column chromatography on silica gel (Combiflash®, 2.5% methanol in DCM). MS (ES): m/z 594.6 [M+H]⁺.

Synthesis of compound I-158. Compound I-158 was prepared from compound 158.7 following the procedure described in the synthesis of compound I-9. The product was purified by flash column chromatography on silica gel (2.4% methanol in DCM). MS (ES): m/z 557.29 [M+H]⁺. ¹H NMR (400 MHz, DMSO-d₆): δ 8.47 (s, 1H), 8.31 (d, J=3.2 Hz, 1H), 7.53 (s, 1H), 7.40 (m, 1H), 6.89 (d, J=7.2 Hz, 1H), 6.80 (d, J=3.2 Hz, 1H), 4.58 (m, 2H), 4.29-4.25 (m, 2H), 3.39 (m, 1H), 3.31-3.30 (m, 6H), 2.52 (m, 4H), 2.01-1.98 (m, 3H), 1.72-1.71 (d, 1H), 1.49-1.46 (m, 1H), 1.38-1.17 (m, 2H).

### Example I-159: (2R,4S)-4-(9-(benzo[d][1,3]dioxol-5-yl)-3-methyl-8-(1-methyl-1H-pyrazol-4-yl)-2-oxo-2,3-dihydro-1H-imidazo[4,5-d]pyrrolo[1,2-b]pyridazin-1-yl)-N-((1s,4S)-4-fluorocyclohexyl)tetrahydrofuran-2-carboxamide

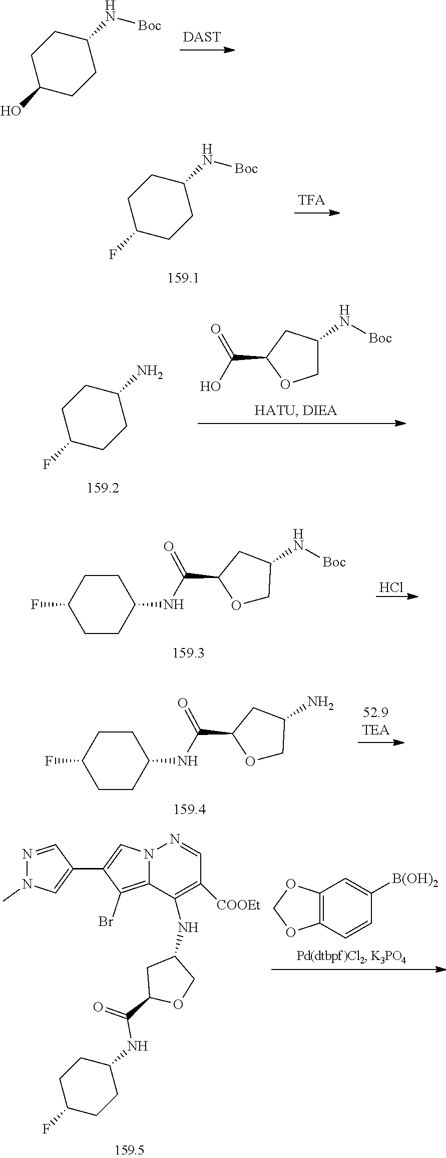

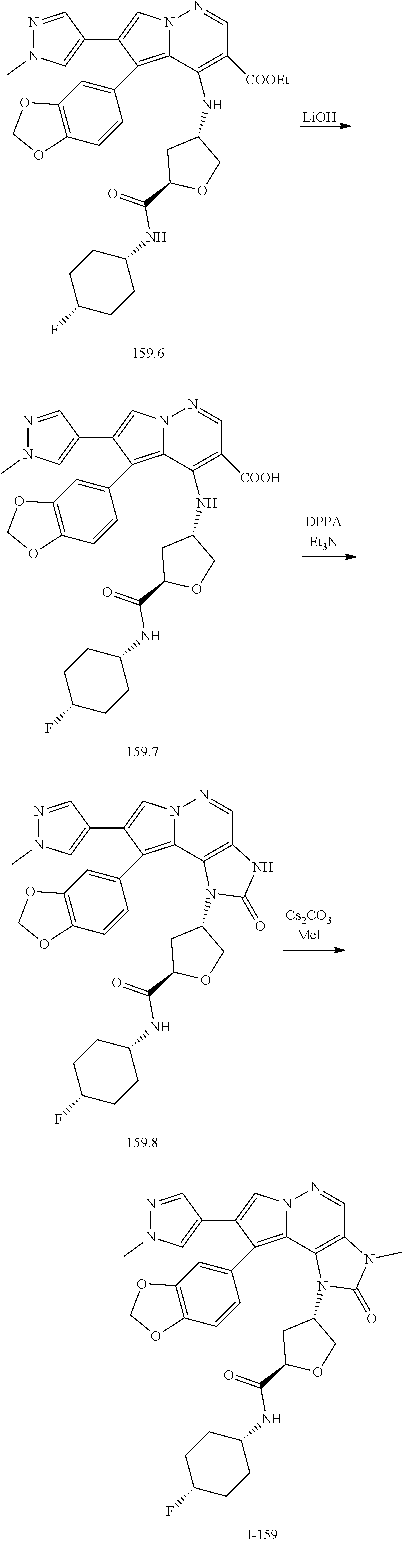

Synthesis of compound 159.1. To a stirred solution of tert-butyl ((1r,4r)-4-hydroxycyclohexyl)carbamate (10 g, 46.45 mmol, 1.0 equiv) in DCM (150 mL) at −78° C. was added dropwise diethylaminosulfur trifluoride (11.23 g, 69.67 mmol, 1.5 equiv) and stirred for 16 h at room temperature. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (20% ethyl acetate in hexane) to afford 159.1 (3.2 g, 32%) MS(ES): m/z 218.28 [M+H]⁺.

Synthesis of compound 159.2. To a solution of 159.1 (3.2 g, 14.86 mmol, 1.0 equiv) in DCM (35 mL) at 0° C. was added dropwise trifluoroacetic acid (10.0 mL) and stirred for 30 min at room temperature. The reaction mixture was concentrated under reduced pressure. It was basified to pH-7 using sodium bicarbonate solution and extracted with ethyl acetate. The combined organic layers were washed with brine, dried over anhydrous sodium sulfate, filtered and concentrated under reduced pressure. The residue was purified by flash column chromatography on silica gel (4.5% methanol in DCM) to afford 159.2 (1.80 g, 56%). MS(ES): m/z 118.18 [M+H]⁺.

Synthesis of compound 159.3. To a solution of (2R,4S)-4-((tert-butoxycarbonyl)amino)tetrahydrofuran-2-carboxylic acid (0.500 g, 2.16 mmol, 1.0 equiv) in DMF (8 mL) was added HATU (1.23 g, 3.24 mmol, 1.5 equiv) and DIPEA (0.836 g, 6.48 mmol, 3 equiv) at 0° C. and stirred for 30 min. To the mixture was added 159.2 (0.506 g, 4.32 mmol, 2.0 equiv) and stirred at room temperature for 1 h. It was transferred into ice-water and extracted with ethyl acetate. The combined organic layers were washed with brine, dried over anhydrous sodium sulfate, filtered and concentrated under reduced pressure. The residue was purified by flash column chromatography on silica gel (4.8% methanol in DCM) to afford 159.3 (0.440 g, 62%). MS(ES): m/z 331.40 [M+H]⁺.

Synthesis of compound 159.4. To a solution of 159.3 (0.440 g, 1.33 mmol, 1.0 equiv) in DCM (6 mL) was added 4 M HCl in dioxane (5 mL) at 0° C. The reaction was stirred at room temperature for 1 h. It was diluted with DCM and concentrated under reduced pressure to afford 159.4 (0.400 g). It was used in the next step without purification.

Synthesis of compound 159.5. Compound 159.5 was prepared from compound 159.4 and 52.9 following the procedure described in the synthesis of compound 1.9. The product was purified by flash column chromatography on silica gel (Combiflash®, 4.8% methanol in DCM). MS(ES): m/z 577.3 and 579.3 [M+H]⁺.

Synthesis of compound 159.6. Compound 159.6 was prepared from compound 159.5 and benzo[d][1,3]dioxol-5-ylboronic acid following the procedure described in the synthesis of compound 59.12. The product was purified by flash column chromatography on silica gel (50% ethyl acetate in n-hexane). MS (ES): m/z 619.5 [M+H]⁺.

Synthesis of compound 159.7. Compound 159.7 was prepared from compound 159.6 following the procedure described in the synthesis of compound 1.10. The product was used in the next step without purification. MS (ES): m/z 591.61 [M+H]⁺.

Synthesis of compound 159.8. Compound 159.8 was prepared from compound 159.7 following the procedure described in the synthesis of compound 1.11. The product was purified by flash column chromatography on silica gel (2.2% methanol in DCM). MS (ES): m/z 588.45 [M+H]⁺.

Synthesis of compound I-159. Compound I-159 was prepared from compound 159.8 following the procedure described in the synthesis of compound 1.12. The product was purified by flash column chromatography on silica gel (2.0% methanol in DCM). MS (ES): m/z 602.3 [M+H]⁺, ¹H-NMR (400 MHz, DMSO-d₆): δ 8.40 (s, 1H), 8.12 (s, 1H), 7.66-7.42 (m, 1H), 7.32 (d, J=7.6 Hz, 1H), 7.17-7.16 (d, J=6.4 Hz, 1H), 6.99 (t, J=8 Hz, 1H), 6.92 (d, J=7.6 Hz, 1H), 6.84 (s, 1H), 6.20-6.18 (m, 1H), 6.07-6.02 (m, 1H), 4.84-4.72 (m, 1H), 4.52-4.48 (m, 1H), 4.15-4.08 (m, 1H), 3.85-3.81 (m, 1H), 3.69 (s, 3H), 3.60-3.56 (m, 2H), 3.39-3.33 (m, 5H), 1.95 (s, 2H), 1.55 (m, 6H).

### Example I-160: 3-((1S,3S)-3-(8-(2,4-dimethyloxazol-5-yl)-9-(4-methoxyphenyl)-3-methyl-2-oxo-2,3-dihydro-1H-imidazo[4,5-d]pyrrolo[1,2-b]pyridazin-1-yl)cyclopentyl)oxazolidin-2-one

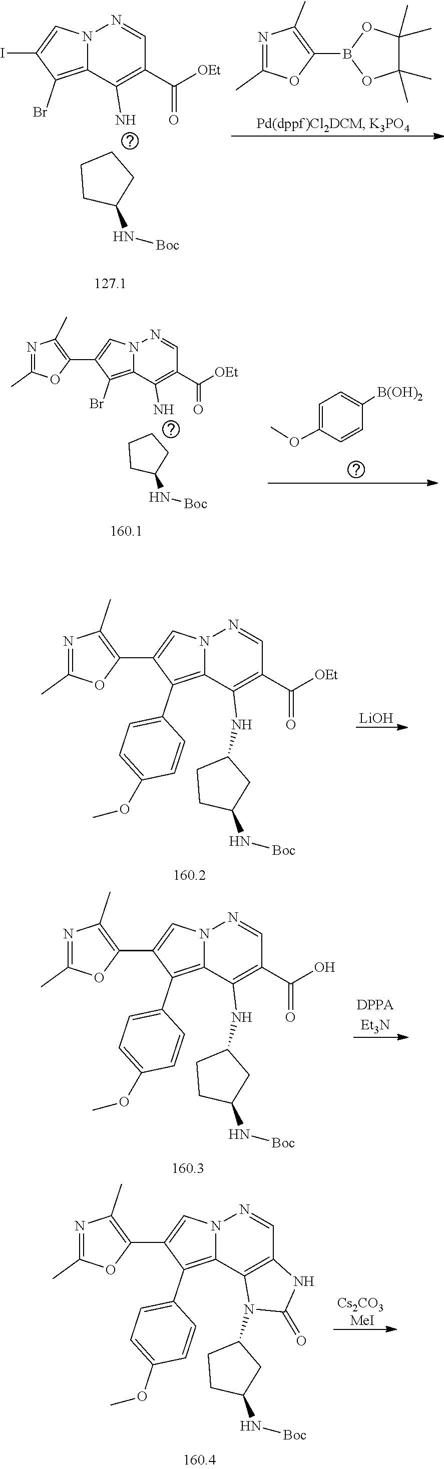

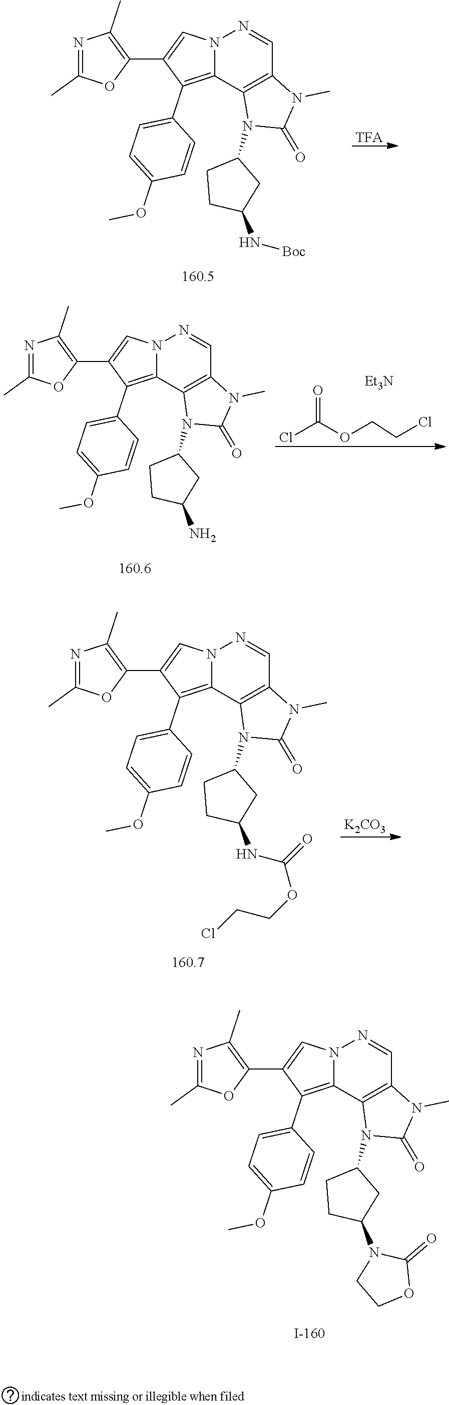

Synthesis of compound 160.1. Compound 160.1 was prepared from compound 127.1 and 2,4-dimethyl-5-(4,4,5,5-tetramethyl-1,3,2-dioxaborolan-2-yl)oxazole following the procedure described in the synthesis of compound 54.3. The product was purified by flash column chromatography on silica gel (Combiflash®, 42% ethyl acetate in n-hexane). MS(ES): m/z 562.6 and 564.5 [M+H]⁺.

Synthesis of compound 160.2. Compound 160.2 was prepared from compound 160.1 and (4-methoxyphenyl)boronic acid following the procedure described in the synthesis of compound 59.12. The product was purified by flash column chromatography on silica gel (60% ethyl acetate in n-hexane). MS (ES): m/z 590.3 [M+H]⁺.

Synthesis of compound 160.3. Compound 160.3 was prepared from compound 160.2 following the procedure described in the synthesis of compound 1.10. The product was used in the next step without purification. MS (ES): m/z 562.5 [M+H]⁺.

Synthesis of compound 160.4. Compound 160.4 was prepared from compound 160.3 following the procedure described in the synthesis of compound 1.11. The product was purified by flash column chromatography on silica gel (2.2% methanol in DCM). MS (ES): m/z 559.4 [M+H]⁺.

Synthesis of compound 160.5. Compound 160.5 was prepared from compound 160.4 following the procedure described in the synthesis of compound 1.12. The product was purified by flash column chromatography on silica gel (2.0% methanol in DCM). MS (ES): m/z 573.2 [M+H]⁺.

Synthesis of compound 160.6. Compound 160.6 was prepared from compound 160.5 following the procedure described in the synthesis of compound 1.14. The product was used in the next step without purification. MS (ES): m/z 473.4 [M+H]⁺.

Synthesis of compound 160.7. Compound 160.7 was prepared from compound 160.6 and 2-chloroethyl carbonochloridate following the procedure described in the synthesis of compound 9.7. The product was purified by flash column chromatography on silica gel (Combiflash®, 3.0% methanol in DCM). MS(ES): m/z 579.4 [M+H]⁺.

Synthesis of I-160. Compound I-160 was prepared from compound 160.7 following the procedure described in the synthesis of compound I-9. The product was purified by flash column chromatography on silica gel (Combiflash®, 3.5% methanol in DCM). MS(ES): m/z 543.4. ¹H-NMR (400 MHz, DMSO-d₆): δ 8.51 (s, 1H), 8.11 (s, 1H), 7.31 (t, J=9.2 Hz, 2H), 7.02-7.07 (m, 2H), 4.33 (m, 3H), 3.83 (m, 1H), 3.85 (s, 3H), 3.39 (s, 3H), 3.23 (t, J=2.4 Hz, 2H), 2.19 (s, 3H), 2.14 (m, 2H), 1.94 (s, 3H), 1.73 (m, 1H), 1.51 (m, 1H), 1.39 (m, 1H), 0.93 (m, 1H).

### Example I-161: (1r,4r)-4-(9-(benzo[d][1,3]dioxol-5-yl)-3-methyl-8-(1-methyl-1H-pyrazol-4-yl)-2-oxo-2,3-dihydro-1H-imidazo[4,5-d]pyrrolo[1,2-b]pyridazin-1-yl)-N-methylcyclohexane-1-carboxamide

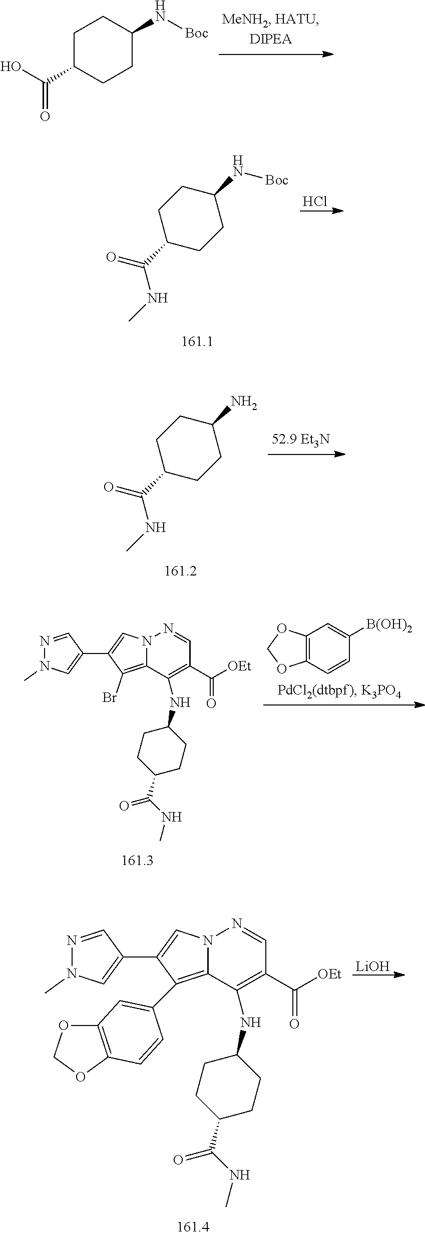

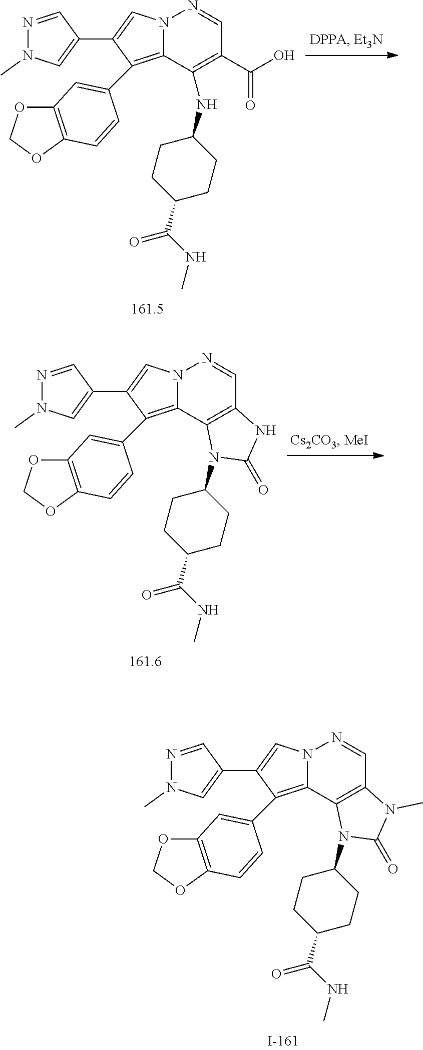

Synthesis of compound 161.1. To a solution of (1r,4r)-4-((tert-butoxycarbonyl)amino)cyclohexane-1-carboxylic acid (1.50 g, 6.17 mmol, 1.0 equiv) in DMF (15 mL) was added HATU (3.51 g, 9.26 mmol, 1.5 equiv) at 0° C. and stirred for 30 min. To this reaction mixture were added N,N-diisopropylethylamine (2.39 g, 18.818 mmol, 3.0 equiv) and methylamine (2 M in THF, 9 mL, 18.52 mmol, 3.0 equiv) at 0° C. It was transferred into water and extracted with ethyl acetate, washed with brine, dried over anhydrous sodium sulfate, filtered and concentrated under reduced pressure. The residue was purified by flash column chromatography on silica gel (Combiflash®, 20% ethyl acetate in hexane) to afford 161.1 (1.01 g, 64%). MS(ES): m/z 256.3 [M+H]⁺.

Synthesis of compound 161.2. To a solution of 161.1 (1.01 g, 3.94 mmol, 1.0 equiv) in DCM (12 mL) was added 4 M HCl in 1,4-dioxane (3.03 mL) dropwise at 0° C. The reaction mixture was stirred at room temperature for 1 h. The reaction mixture was neutralized by sodium bicarbonate solution and extracted with DCM. The combined organic layers were dried over anhydrous sodium sulfate, filtered and dried under reduced pressure to afford 161.2 (0.352 g, 58%). MS(ES), m/z 156.23 [M+H]⁺.

Synthesis of compound 161.3. Compound 161.3 was prepared from compound 161.2 and 52.9 following the procedure described in the synthesis of compound 1.9. The product was purified by flash column chromatography on silica gel (30% ethyl acetate in hexane). MS(ES): m/z 503.3 and 505.5 [M+H]⁺.

Synthesis of compound 161.4. Compound 161.4 was prepared from compound 161.3 and benzo[d][1,3]dioxol-5-ylboronic acid following the procedure described in the synthesis of compound 59.12. The product was purified by flash column chromatography on silica gel (55% ethyl acetate in hexane). MS(ES): m/z 545.5 [M+H]⁺.

Synthesis of compound 161.5. Compound 161.5 was prepared from compound 161.4 following the procedure described in the synthesis of compound 1.10. The product was triturated with pentane, dried under vacuum and used in the next step without purification. MS(ES): m/z 517.4 [M+H]⁺.

Synthesis of compound 161.6. Compound 161.6 was prepared from compound 161.5 following the procedure described in the synthesis of compound 1.11. The product was purified by flash column chromatography on silica gel (Combiflash®, 2.1% methanol in DCM). MS(ES): m/z 514.56 [M+H]⁺.

Synthesis of I-161. Compound I-161 was prepared from compound 161.6 following the procedure described in the synthesis of compound 1.12. The product was purified by flash column chromatography on silica gel (1.7% methanol in DCM). MS(ES): m/z 528.44 [M+H]⁺. ¹H-NMR (400 MHz, DMSO-d₆): δ 8.39 (s, 1H), 8.12 (s, 1H), 7.67 (d, J=4.4 Hz, 1H), 7.26 (s, 1H), 7.10 (s, 1H), 7.01 (d, J=8.0 Hz, 1H), 6.95 (s, 1H), 6.78 (d, J=1.6 Hz, 1H), 6.28 (d, J=5.2 Hz, 2H), 3.74 (s, 3H), 3.52-3.48 (m, 1H), 2.56-2.54 (m, 5H), 2.34-2.34 (m, 1H), 2.21-2.18 (m, 1H), 1.92 (t, J=12.4 Hz, 1H), 1.95-1.89 (m, 4H), 1.47 (d, J=12.0 Hz, 1H), 1.02-0.99 (m, 1H), 0.77-0.71 (m, 1H).

### Example I-162: (1R,2R)—N-((1s,4S)-4-(9-(2,3-dihydro-[1,4]dioxino[2,3-b]pyridin-7-yl)-3-methyl-8-(1-methyl-1H-pyrazol-4-yl)-2-oxo-2,3-dihydro-1H-imidazo[4,5-d]pyrrolo[1,2-b]pyridazin-1-yl)-1-methylcyclohexyl)-2-fluorocyclopropane-1-carboxamide

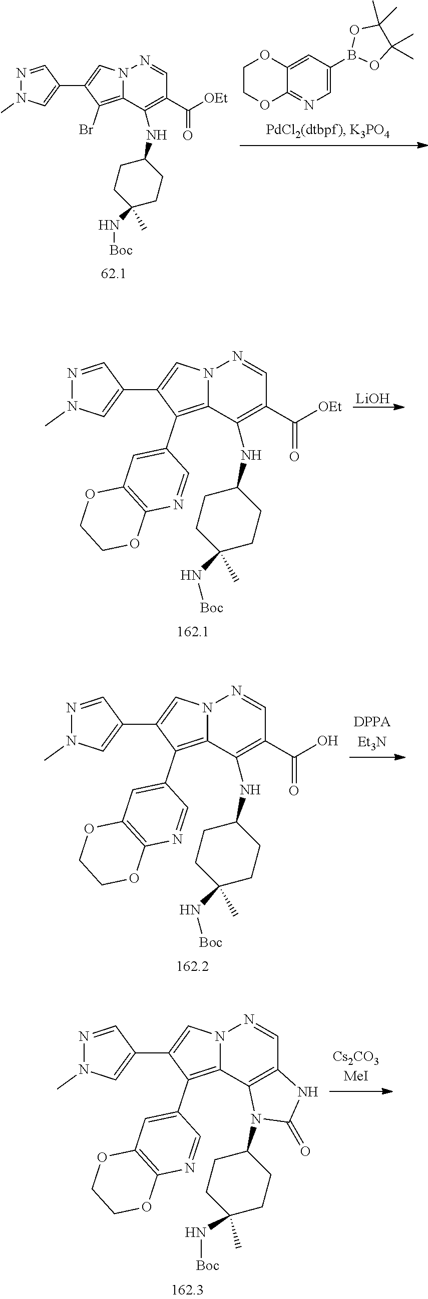

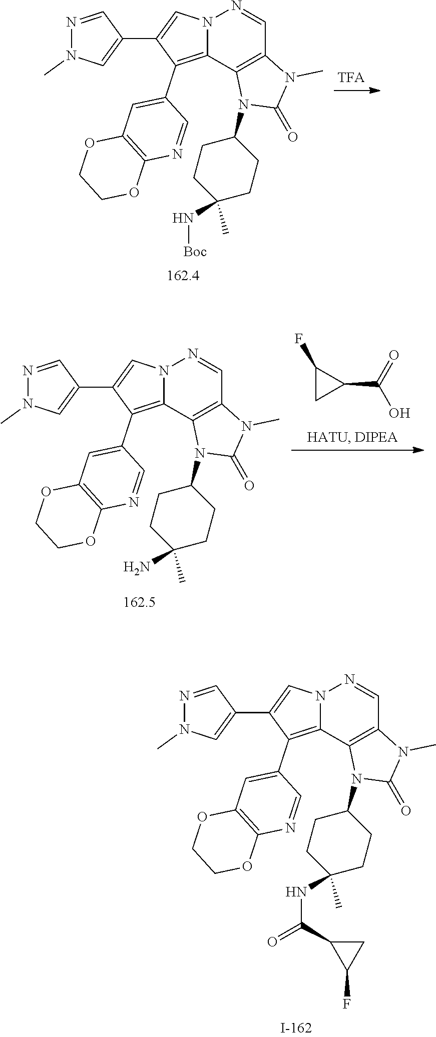

Synthesis of compound 162.1. Compound 162.1 was prepared from compound 62.1.

Synthesis of compound 162.1. Compound 162.1 was prepared from compound 62.1 and 7-(4,4,5,5-tetramethyl-1,3,2-dioxaborolan-2-yl)-2,3-dihydro-[1,4]dioxino[2,3-b]pyridine following the procedure described in the synthesis of compound 59.12. The product was purified by flash column chromatography on silica gel (95% ethyl acetate in hexane). MS(ES): m/z 632.4 [M+H]⁺.

Synthesis of compound 162.2. Compound 162.2 was prepared from compound 162.1 following the procedure described in the synthesis of compound 1.10. The product was triturated with pentane, dried under vacuum and used in the next step without purification. MS(ES): m/z 604.3 [M+H]⁺.

Synthesis of compound 162.3. Compound 162.3 was prepared from compound 162.2 following the procedure described in the synthesis of compound 1.11. The product was purified by flash column chromatography on silica gel (Combiflash®, 2.0% methanol in DCM). MS(ES): m/z 601.3 [M+H]⁺.

Synthesis of compound 162.4. Compound 162.4 was prepared from compound 162.3 following the procedure described in the synthesis of compound 1.12. The product was purified by flash column chromatography on silica gel (1.8% methanol in DCM). MS(ES): m/z 615.6 [M+H]⁺.

Synthesis of compound 162.5. Compound 162.5 was prepared from compound 162.4 following the procedure described in the synthesis of compound 1.14. The product was purified by flash column chromatography on silica gel (1.8% methanol in DCM). MS(ES): m/z 515.3 [M+H]⁺.

Synthesis of compound I-162. Compound I-162 was prepared from compound 162.5 and (1R,2R)-2-fluorocyclopropane-1-carboxylic acid following the procedure described in the synthesis of compound I-45. The product was purified by flash column chromatography on silica gel (3.5% methanol in DCM). MS(ES): m/z 601.20 [M+H]⁺. ¹H-NMR (400 MHz, DMSO-d₆): δ 8.42 (s, 1H), 8.15 (s, 1H), 7.78 (d, J=2.0 Hz, 1H), 7.51 (s, 1H), 7.37 (d, J=2.8 Hz, 1H), 7.27 (d, J=6.8 Hz, 1H), 7.14 (s, 1H), 4.84-4.81 (m, 1H), 4.69-4.67 (m, 1H), 4.59-4.56 (m, 1H), 4.47-4.40 (m, 2H), 4.29-4.25 (m, 1H), 3.76 (s, 3H), 3.37 (s, 3H), 3.35-3.30 (m, 1H), 2.15-2.12 (m, 1H), 1.88-1.82 (m, 1H), 1.49-1.46 (m, 1H), 1.31 (m, 2H), 1.24 (m, 2H), 1.15 (s, 3H), 0.99-0.92 (m, 1H), 0.51-0.37 (m, 2H).

### Example I-163: (1R,2R)-2-fluoro-N-((1s,4S)-1-methyl-4-(3-methyl-9-(1-methyl-1H-indazol-5-yl)-8-(4-methyloxazol-5-yl)-2-oxo-2,3-dihydro-1H-imidazo[4,5-d]pyrrolo[1,2-b]pyridazin-1-yl)cyclohexyl)cyclopropane-1-carboxamide

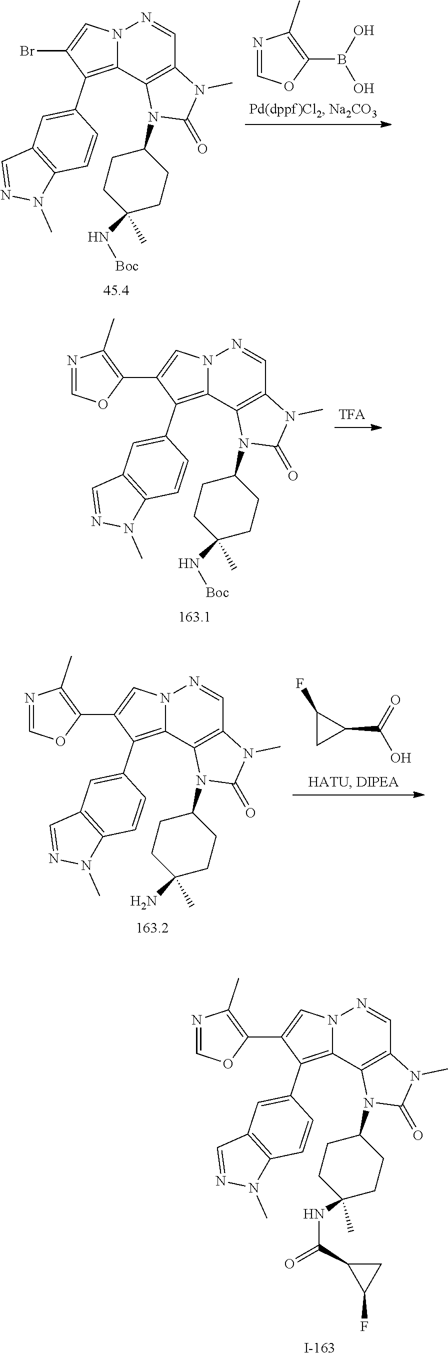

Synthesis of compound 163.1. Compound 163.1 was prepared from compound 45.4 and (4-methyloxazol-5-yl)boronic acid following the procedure described in the synthesis of compound 1.13. The product was purified by flash column chromatography on silica gel (Combiflash®, 3.1% methanol in DCM). MS(ES): m/z 611.37 [M+H]⁺.

Synthesis of compound 163.2. Compound 163.2 was prepared from compound 163.1 following the procedure described in the synthesis of compound 1.14. The product was used in the next step without purification. MS(ES): m/z 511.37 [M+H]⁺.

Synthesis of I-163. Compound I-163 was prepared from compound 163.2 and (1R,2R)-2-fluorocyclopropane-1-carboxylic acid following the procedure described in the synthesis of compound I-45. The product was purified by silica gel flash column chromatography on silica gel (3.7% methanol in DCM). MS(ES): m/z 597.26 [M+H]⁺. ¹H-NMR (400 MHz, DMSO-d₆): δ 8.52 (s, 1H), 8.19 (s, 1H), 8.07-8.02 (m, 2H), 7.74 (s, 1H), 7.72 (s, 1H), 7.38-7.29 (m, 2H), 4.78-4.61 (m, 1H), 4.09 (s, 3H), 3.39 (s, 3H), 3.05 (s, 1H), 2.35-2.32 (m, 2H), 2.01 (s, 3H), 1.85-1.75 (m, 4H), 1.42-1.27 (m, 2H), 1.01-0.90 (m, 2H), 0.67 (s, 3H), −0.105 (s, 1H).

### Example I-164: 3-((1S,3S)-3-(9-(6,7-dihydro-4H-thieno[3,2-c]pyran-2-yl)-8-(2,4-dimethyloxazol-5-yl)-3-methyl-2-oxo-2,3-dihydro-1H-imidazo[4,5-d]pyrrolo[1,2-b]pyridazin-1-yl)cyclopentyl)oxazolidin-2-one

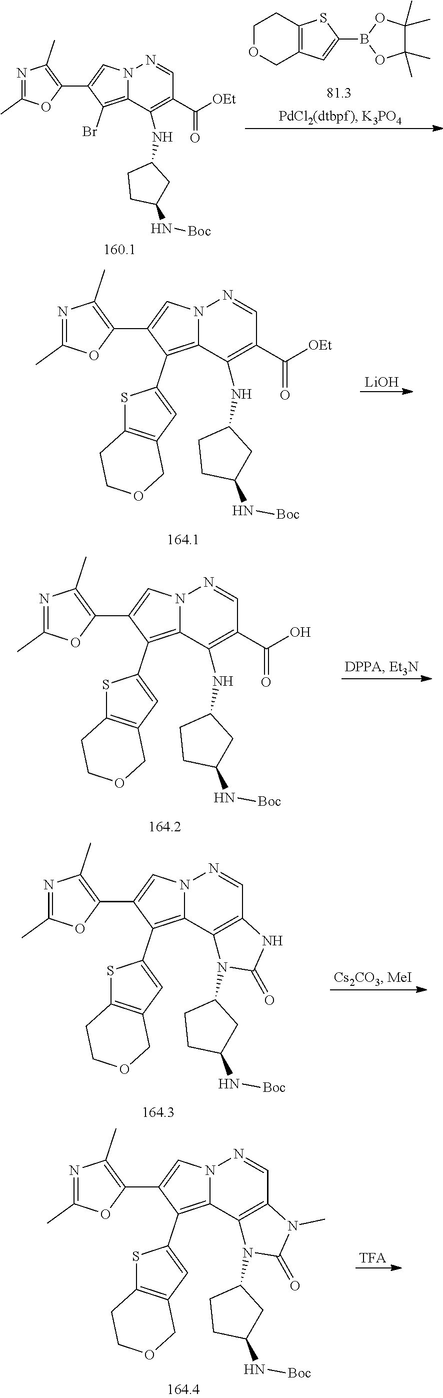

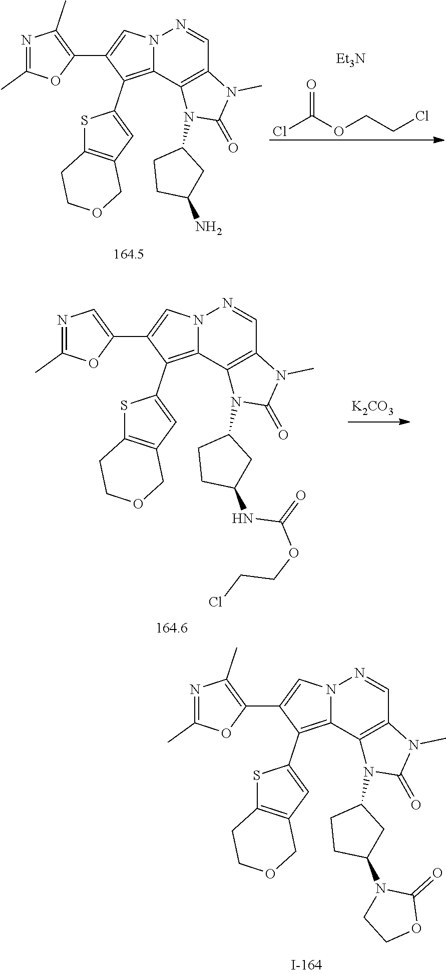

Synthesis of compound 164.1. Compound 164.1 was prepared from compound 160.1 and 81.3 following the procedure described in the synthesis of compound 59.12. The product was purified by flash column chromatography on silica gel (40% ethyl acetate in n-hexane). MS (ES): m/z 622.7 [M+H]⁺.

Synthesis of compound 164.2. Compound 164.2 was prepared from compound 164.1 following the procedure described in the synthesis of compound 1.10. The product was used in the next step without purification. MS (ES): m/z 594.4 [M+H]⁺.

Synthesis of compound 164.3. Compound 164.3 was prepared from compound 164.2 following the procedure described in the synthesis of compound 1.11. The product was purified by flash column chromatography on silica gel (3.5% methanol in DCM). MS (ES): m/z 591.7 [M+H]⁺.

Synthesis of compound 164.4. Compound 164.4 was prepared from compound 164.3 following the procedure described in the synthesis of compound 1.12. The product was purified by flash column chromatography on silica gel (2.7% methanol in DCM). MS (ES): m/z 605.5 [M+H]⁺.

Synthesis of compound 164.5. Compound 164.5 was prepared from compound 164.4 following the procedure described in the synthesis of compound 1.14. The product was used in the next step without purification. MS (ES): m/z 505.6 [M+H]⁺.

Synthesis of compound 164.6. Compound 164.6 was prepared from compound 164.5 and 2-chloroethyl carbonochloridate following the procedure described in the synthesis of compound 9.7. The product was purified by flash column chromatography on silica gel (Combiflash®, 2.5% methanol in DCM). MS (ES): m/z 612.1 [M+H]⁺.

Synthesis of compound I-164. Compound I-164 was prepared from compound 164.6 following the procedure described in the synthesis of compound I-9. The product was purified by flash column chromatography on silica gel (2.4% methanol in DCM). MS (ES): m/z 575.17 [M+H]⁺. ¹H NMR (400 MHz, DMSO-d₆): δ 8.58 (s, 1H), 8.14 (s, 1H), 6.90 (s, 1H), 4.64-4.56 (m, 2H), 4.45-4.40 (m, 1H), 4.30-4.22 (m, 2H), 3.96 (s, 3H), 3.42 (s, 3H), 2.85 (s, 2H), 2.28 (s, 3H), 2.12 (d, J=4.4 Hz, 2H), 2.02 (s, 3H), 1.84 (t, J=6.0 Hz, 1H), 1.67 (d, J=4.4 Hz, 1H), 1.56 (d, J=8.4 Hz, 1H), 1.24 (d, J=3.6 Hz, 3H), 1.21-1.20 (m, 1H).

### Example I-165: 3-((1S,3S)-3-(9-(2,3-dihydrobenzo[b][1,4]dioxin-6-yl)-8-(3-fluoro-1-methyl-1H-pyrazol-4-yl)-3-methyl-2-oxo-2,3-dihydro-1H-imidazo[4,5-d]pyrrolo[1,2-b]pyridazin-1-yl)cyclopentyl)oxazolidin-2-one

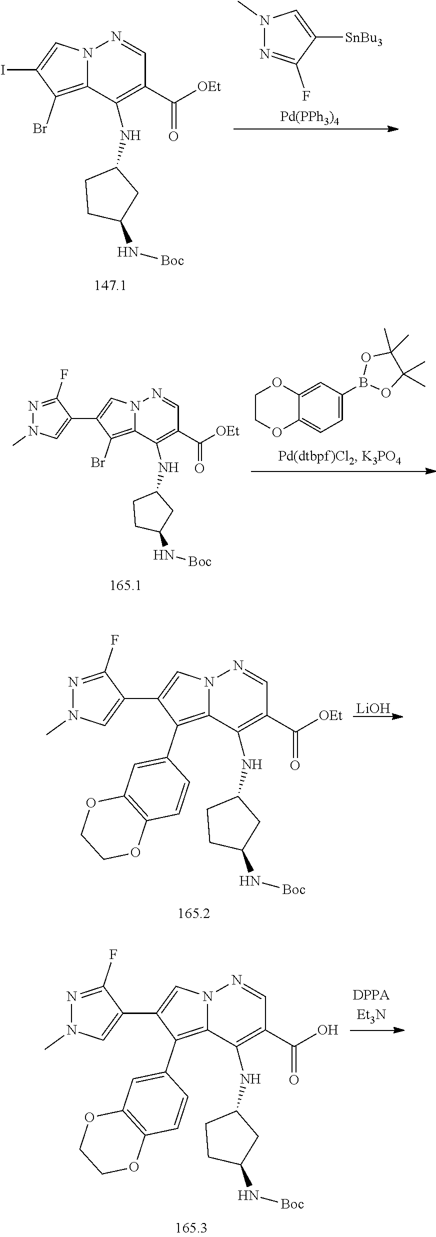

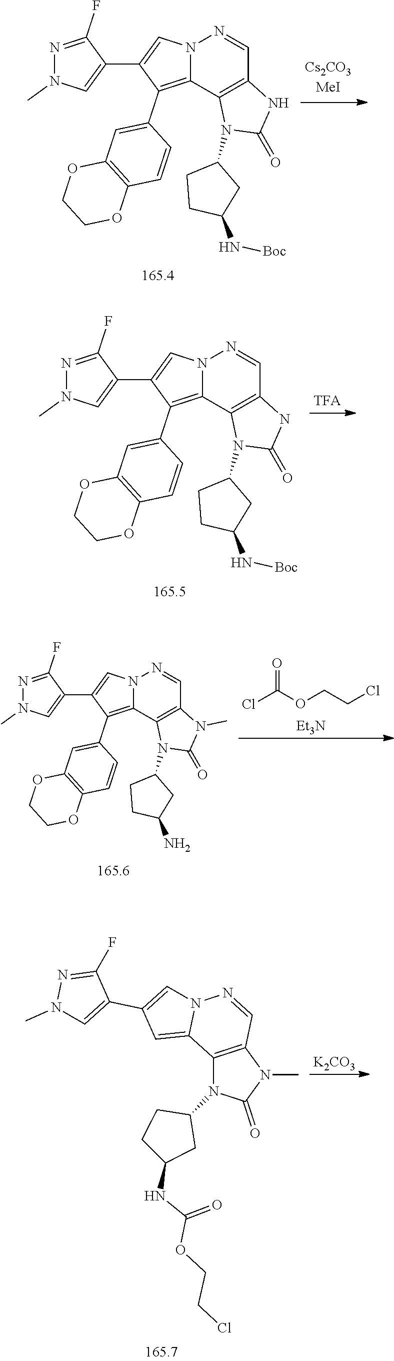

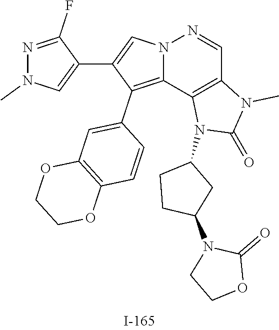

Synthesis of compound 165.1. A solution of 147.1 (1.0 g, 1.69 mmol, 1.0 equiv) and 3-fluoro-1-methyl-4-(tributylstannyl)-1H-pyrazole (0.418 g, 2.53 mmol, 1.5 equiv) in 1,4-dioxane (15.0 mL) was added was degassed by bubbling through a stream of argon for 10 min. Tetrakis(triphenylphosphine)palladium (0.195 g, 0.168 mmol, 0.1 equiv) was added and degassed for 5 min. The reaction mixture was stirred at room temperature for 2 h. It was filtered through a pad of Celite® and rinsed with ethyl acetate. The filtrate was transferred into water and extracted with ethyl acetate. The combined organic layers were washed with brine, dried over sodium sulfate, filtered and concentrated under reduced pressure. The residue was purified by flash column chromatography on silica gel (3.2% methanol in DCM) to afford 165.2 (0.270 g, 28%). MS(ES): m/z 565.15 and 567.2 [M+H]⁺.

Synthesis of compound 165.2. Compound 165.2 was prepared from compound 165.1 and 2-(2,3-dihydrobenzo[b][1,4]dioxin-6-yl)-4,4,5,5-tetramethyl-1,3,2-dioxaborolane following the procedure described in the synthesis of compound 59.12. The product was purified by flash column chromatography on silica gel (55% ethyl acetate in hexane). MS(ES): m/z 621.5 [M+H]⁺.

Synthesis of compound 165.3. Compound 165.3 was prepared from compound 165.2 following the procedure described in the synthesis of compound 1.10. The product was triturated with pentane. MS(ES): m/z 593.4 [M+H]⁺.

Synthesis of compound 165.4. Compound 165.4 was prepared from compound 165.3 following the procedure described in the synthesis of compound 1.11. The product was purified by flash column chromatography on silica gel (Combiflash®, 3.8% methanol in DCM). MS(ES): m/z 590.25 [M+H]⁺.

Synthesis of compound 165.5. Compound 165.5 was prepared from compound 165.4 following the procedure described in the synthesis of compound 1.12. The product was purified by flash column chromatography on silica gel (2.4% methanol in DCM). MS(ES): m/z 604.26 [M+H]⁺.

Synthesis of compound 165.6. Compound 165.6 was prepared from compound 165.5 following the procedure described in the synthesis of compound 1.14. The product was triturated with n-pentane and dried under reduced pressure. It was used in the next step without further purification. MS(ES): m/z 504.21 [M+H]⁺.

Synthesis of compound 165.7. Compound 165.7 was prepared from compound 165.6 and 2-chloroethyl carbonochloridate following the procedure described in the synthesis of compound 9.7. The product was purified by flash column chromatography on silica gel (2.0% methanol in DCM). MS(ES): m/z 611.14 [M+H]⁺.

Synthesis of compound I-165. Compound I-165 was prepared from compound 165.7 following the procedure described in the synthesis of compound I-9. The product was purified by flash column chromatography on silica gel (Combiflash®, 3.4% methanol in DCM). MS (ES): m/z 574.27 [M+H]⁺; ¹H-NMR (400 MHz, DMSO-d6): δ 8.43 (s, 1H), 7.94 (s, 1H), 7.06-7.04 (m, 1H), 6.99-6.80 (m, 3H), 4.33-4.27 (m, 8H), 3.99-3.91 (m, 1H), 3.67 (s, 3H), 2.07 (m, 3H), 1.77 (m, 1H), 1.54 (m, 2H), 1.33 (m, 1H), 1.11-1.03 (m, 2H), 0.85 (m, 1H).

### Example I-166-a and I-166-b: (1r,4R)-4-(9-(benzo[d][1,3]dioxol-5-yl)-3-methyl-8-(1-methyl-1H-pyrazol-4-yl)-2-oxo-2,3-dihydro-1H-imidazo[4,5-d]pyrrolo[1,2-b]pyridazin-1-yl)-N—(((R)-tetrahydrofuran-3-yl)methyl)cyclohexane-1-carboxamide and (1r,4S)-4-(9-(benzo[d][1,3]dioxol-5-yl)-3-methyl-8-(1-methyl-1H-pyrazol-4-yl)-2-oxo-2,3-dihydro-1H-imidazo[4,5-d]pyrrolo[1,2-b]pyridazin-1-yl)-N—(((S)-tetrahydrofuran-3-yl)methyl)cyclohexane-1-carboxamide

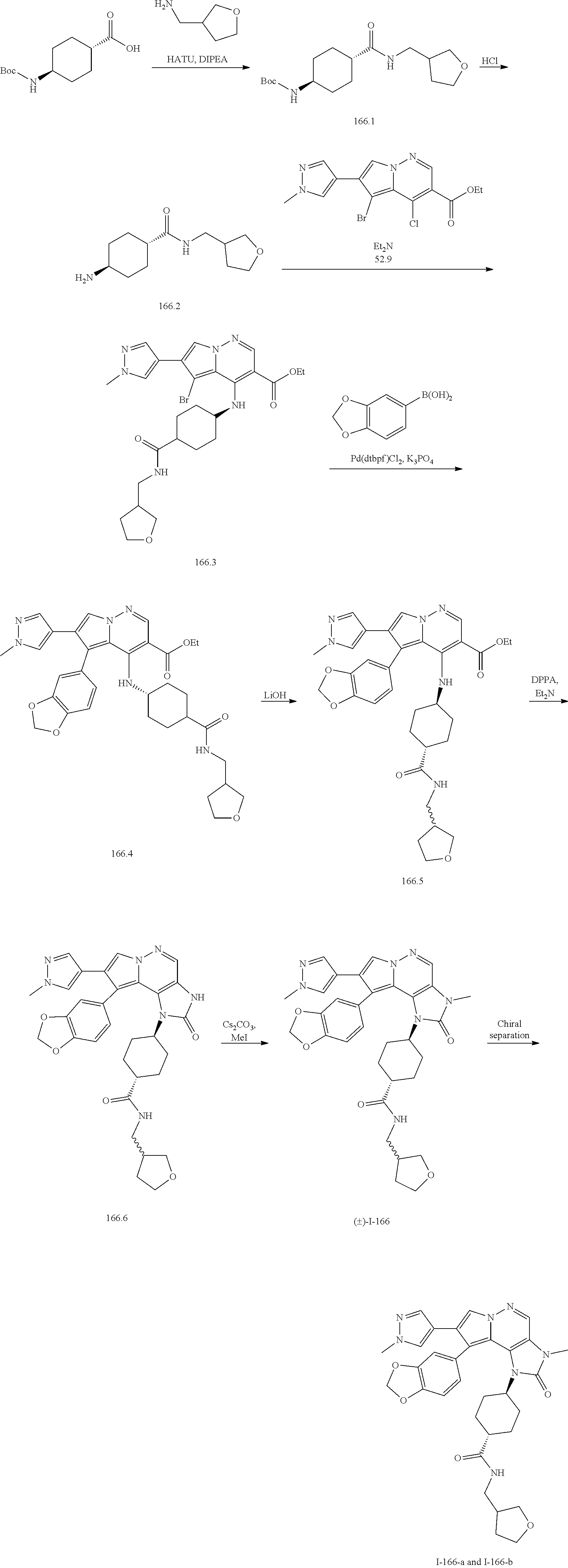

Synthesis of compound 166.1. A solution of (1r,4r)-4-((tert-butoxycarbonyl)amino)cyclohexane-1-carboxylic acid (1.1 g, 4.52 mmol, 1.0 equiv) and HATU (2.58 g, 6.79 mmol, 1.5 equiv) in DMF (10 mL) was stirred at 0° C. for 30 min. To the reaction mixture were added N, N-diisopropylethylamine (1.75 g, 13.580 mmol, 3.0 equiv) and (tetrahydrofuran-3-yl)methanamine (1.37 g, 13.58 mmol, 3.0 equiv) at 0° C. and stirred for 30 min. It transferred into water and extracted with ethyl acetate. The organic layer was washed with brine, dried over sodium sulfate, filtered and concentrated under reduced pressure. The residue was purified by flash column chromatography on silica gel (Combiflash®, 3.2% methanol in DCM) to afford 166.1 (0.691 g, 47%).

Synthesis of compound 166.2. To a solution of 166.1 (0.691 g, 2.12 mmol, 1.0 equiv) in DCM (8 mL) was added a solution of hydrochloric acid in 1,4-dioxane (4 M, 2.07 mL) dropwise at 0° C. The reaction mixture was stirred at room temperature for 1 h. It was concentrated under reduced pressure to afford 166.2 (0.371 g, 77.44%).

Synthesis of compound 166.3. Compound 166.3 was prepared from compound 52.9 and 166.2 following the procedure described in the synthesis of compound 1.9. The product was purified by flash column chromatography on silica gel (30% ethyl acetate in hexane). MS(ES): m/z 574.49 [M+H]⁺.

Synthesis of compound 166.4. Compound 166.4 was prepared from compound 166.3 and benzo[d][1,3]dioxol-5-ylboronic acid following the procedure described in the synthesis of compound 59.12. The product was purified by flash column chromatography on silica gel (55% ethyl acetate in hexane). MS(ES): m/z 615.70 [M+H]⁺.

Synthesis of compound 166.5. Compound 166.5 was prepared from compound 166.4 following the procedure described in the synthesis of compound 1.10. The product was triturated with pentane, dried under reduced pressure and used in the next step without purification. MS(ES): m/z 587.6 [M+H]⁺.

Synthesis of compound 166.6. Compound 166.6 was prepared from compound 166.5 following the procedure described in the synthesis of compound 1.11. The product was purified by flash column chromatography on silica gel (Combiflash®, 2.1% methanol in DCM). MS(ES): m/z 584.65 [M+H]⁺.

Synthesis of compound (±)—I-166. Compound (±)—I-166 was prepared from compound 166.6 following the procedure described in the synthesis of compound 1.12. The product was purified by flash column chromatography on silica gel (1.8% methanol in DCM). MS(ES) 598.26 [M+H]⁺.

Synthesis of compound I-166-a and I-166-b. The racemate was separated by SFC (column: CHIRALPAK IB-N(250*30 mm), 5 μm; mobile phase: (A) Liquid CO₂. (B) 0.1% NH₃ in propane-2-ol: MeCN (50:50); flow rate: 100 mL/min) to afford first eluting fraction (I-166-a) and second eluting fraction (I-166-b). *The absolute configuration of the chiral center is not determined.

I-166-a: MS(ES): m/z 598.26 [M+H]⁺, ¹H-NMR (400 MHz, DMSO-d6): δ 8.38 (s, 1H), 8.11 (s, 1H), 7.82 (t, J=6.00 Hz, 1H), 7.25 (s, 1H), 7.09 (s, 1H), 7.00 (d, J=8.00 Hz, 1H), 6.94 (s, 1H), 6.78 (dd, J=2.00, 7.80 Hz, 1H), 6.25 (d, J=8.80 Hz, 2H), 3.72 (s, 3H), 3.69-3.59 (m, 3H), 3.57-3.49 (m, 1H), 3.47 (s, 3H), 3.03-2.99 (m, 2H), 2.34-2.27 (m, 3H), 1.92-1.87 (m, 2H), 1.54-1.48 (m, 4H), 1.46-1.45 (m, 1H), 1.44-1.24 (m, 1H), 1.06-0.98 (m, 1H), 0.75-0.67 (m, 1H).

I-166-b: MS(ES): m/z 598.32 [M+H]⁺, ¹H-NMR (400 MHz, DMSO-d6): δ 8.38 (s, 1H), 8.11 (s, 1H), 7.83 (t, J=5.60 Hz, 1H), 7.25 (s, 1H), 7.09 (s, 1H), 7.00 (d, J=8.00 Hz, 1H), 6.94 (s, 1H), 6.78 (dd, J=1.60, 8.00 Hz, 1H), 6.25 (d, J=8.80 Hz, 2H), 3.72 (s, 3H), 3.70-3.63 (m, 3H), 3.61-3.48 (m, 1H), 3.37 (s, 1H), 3.04-2.99 (m, 2H), 2.36-2.31 (m, 2H), 2.29-2.27 (m, 2H), 1.94-1.87 (m, 3H), 1.94-1.87 (m, 4H), 1.48-1.46 (m, 1H), 1.39 (s, 1H), 0.96-1.00 (m, 1H), 0.76-0.69 (m, 1H).

### Example I-167: N-methyl-N-(((1r,3r)-3-(3-methyl-9-(1-methyl-1H-indazol-5-yl)-8-(1-methyl-1H-pyrazol-4-yl)-2-oxo-2,3-dihydro-1H-imidazo[4,5-d]pyrrolo[1,2-b]pyridazin-1-yl)cyclobutyl)methyl)cyclopropanecarboxamide

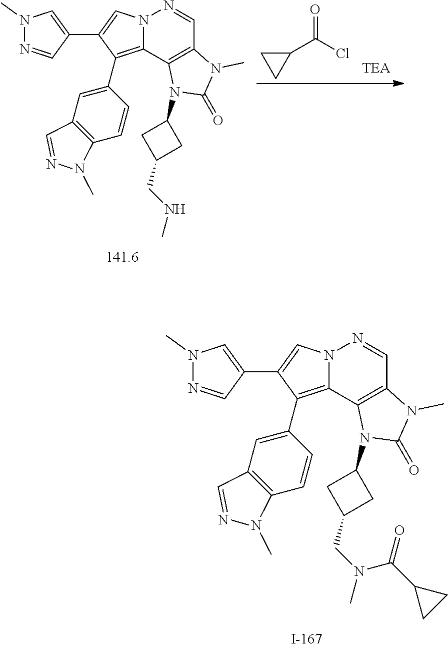

Synthesis of compound I-167. To a solution of 141.6 (0.080 g, 0.16 mmol, 1.0 equiv) in DCM (2.0 mL) was added cyclopropanecarbonyl chloride (0.033 g, 0.32 mmol, 2.0 equiv) and triethylamine (0.067 mL, 0.48 mmol, 3.0 equiv) at 0° C. The reaction mixture was stirred at room temperature for 1 h. It was transferred into ice-water and extracted with ethyl acetate. The combined organic layers were washed with brine, dried over sodium sulfate, filtered and concentrated under reduced pressure. The residue was purified by flash column chromatography on silica gel (Combiflash®, 3% methanol in DCM) to afford I-167 (0.028 g, 31%). MS(ES): 564.4 [M+H]⁺; 1H-NMR (400 MHz, DMSO-d6): δ 8.42 (d, J=6.80 Hz, 1H), 8.12 (d, J=15.60 Hz, 2H), 7.87 (d, J=8.40 Hz, 1H), 7.80 (d, J=15.20 Hz, 1H), 7.42 (t, J=8.40 Hz, 1H), 7.28 (s, 1H), 6.97 (d, J=12.80 Hz, 1H), 4.19-4.15 (m, 3H), 3.94-3.87 (m, 1H), 3.68 (s, 3H), 3.38-3.37 (m, 3H), 2.73-2.67 (m, 1H), 2.87-2.72 (m, 2H), 2.70-2.67 (m, 1H), 2.34-2.26 (m, 2H), 2.22-2.21 (bs, 1H), 2.00-1.78 (m, 1H), 1.40-1.28 (m, 3H), 0.72-0.66 (m, 4H).

### Example I-168: 1R,2R)—N-((1s,4S)-4-(9-(benzo[d][1,3]dioxol-5-yl)-3-methyl-8-(2-methylpyrimidin-4-yl)-2-oxo-2,3-dihydro-1H-imidazo[4,5-d]pyrrolo[1,2-b]pyridazin-1-yl)-1-methylcyclohexyl)-2-fluorocyclopropane-1-carboxamide

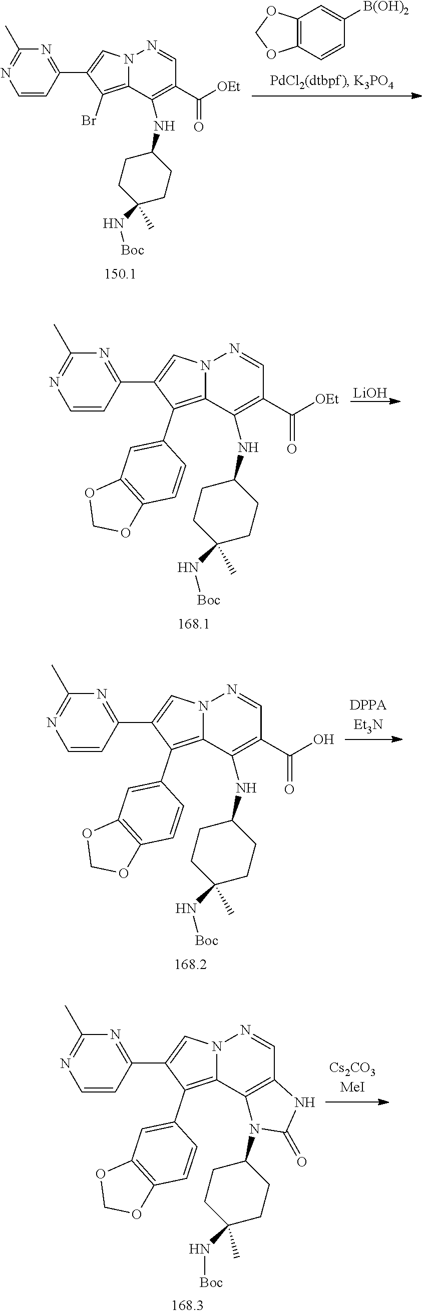

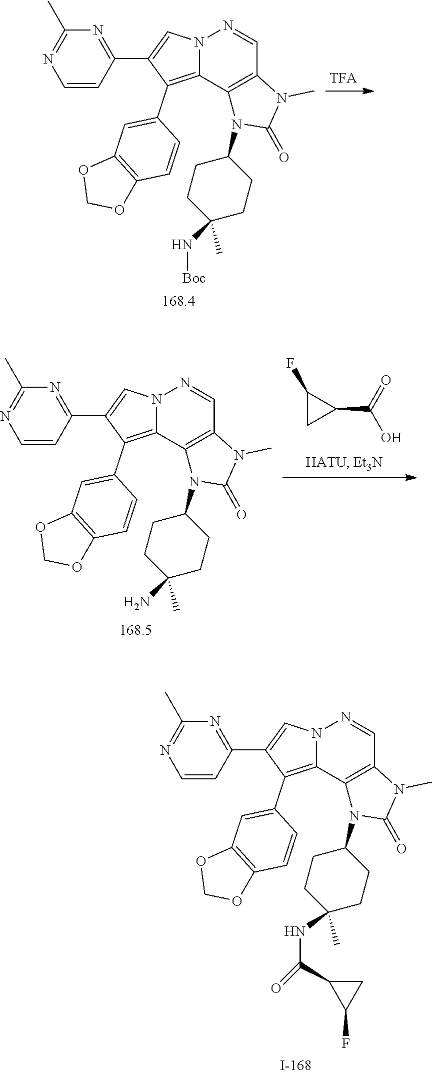

Synthesis of compound 168.1. Compound 168.1 was prepared from compound 150.1 and benzo[d][1,3]dioxol-5-ylboronic acid following the procedure described in the synthesis of compound 59.12. The product was purified by flash column chromatography on silica gel (35% ethyl acetate in hexane). MS(ES): m/z 629.8 [M+H]⁺.

Synthesis of compound 168.2. Compound 162.3 was prepared from compound 162.2 following the procedure described in the synthesis of compound 1.10. The product was triturated with pentane, dried under reduced pressure and used in the next step without purification. MS(ES): m/z 601.5 [M+H]⁺.

Synthesis of compound 168.3. Compound 168.3 was prepared from compound 168.2 following the procedure described in the synthesis of compound 1.11. The product was purified by flash column chromatography on silica gel (Combiflash®, 2.1% methanol in DCM). MS(ES): m/z 598.7 [M+H]⁺.

Synthesis of compound 168.4. Compound 168.4 was prepared from compound 168.3 following the procedure described in the synthesis of compound 1.12. The product was purified by flash column chromatography on silica gel (1.5% methanol in DCM). MS(ES): m/z 612.7 [M+H]⁺.

Synthesis of compound 168.5. Compound 168.5 was prepared from compound 168.4 following the procedure described in the synthesis of compound 1.14. The product was triturated with pentane and dried under reduced pressure. MS(ES): m/z 512.6 [M+H]⁺. It was used in the next step without purification.

Synthesis of compound I-168. Compound I-168 was prepared from compound 168.5 and (1R,2R)-2-fluorocyclopropane-1-carboxylic acid following the procedure described in the synthesis of compound I-45. The product was purified by flash column chromatography on silica gel (3.1% methanol in DCM). MS(ES): m/z 598.3 [M+H]⁺, 1H-NMR (400 MHz, DMSO-d6): δ 8.53 (s, 1H), 8.46 (s, 1H), 8.43 (d, J=5.60 Hz, 1H), 7.49 (s, 1H), 7.10 (d, J=7.20 Hz, 1H), 7.05 (s, 1H), 6.90 (d, J=7.60 Hz, 1H), 6.60 (d, J=5.60 Hz, 1H), 6.15 (s, 2H), 4.84 (m, 0.5H), 4.67-4.66 (m, 0.5H), 3.39 (s, 3H), 2.64-2.61 (m, 1H), 2.54 (s, 3H), 2.19-2.14 (m, 2H), 2.06-1.86 (m, 2H), 1.48-1.43 (m, 2H), 1.35-1.25 (m, 1H), 1.19-1.13 (m, 2H), 0.95 (m, 1H), 0.87-0.85 (m, 1H), 0.55-0.52 (m, 2H).

### Example I-169: (2R,4S)-4-(9-(benzo[d][1,3]dioxol-5-yl)-3-methyl-8-(1-methyl-1H-pyrazol-4-yl)-2-oxo-2,3-dihydro-1H-imidazo[4,5-d]pyrrolo[1,2-b]pyridazin-1-yl)-N-methyltetrahydrofuran-2-carboxamide

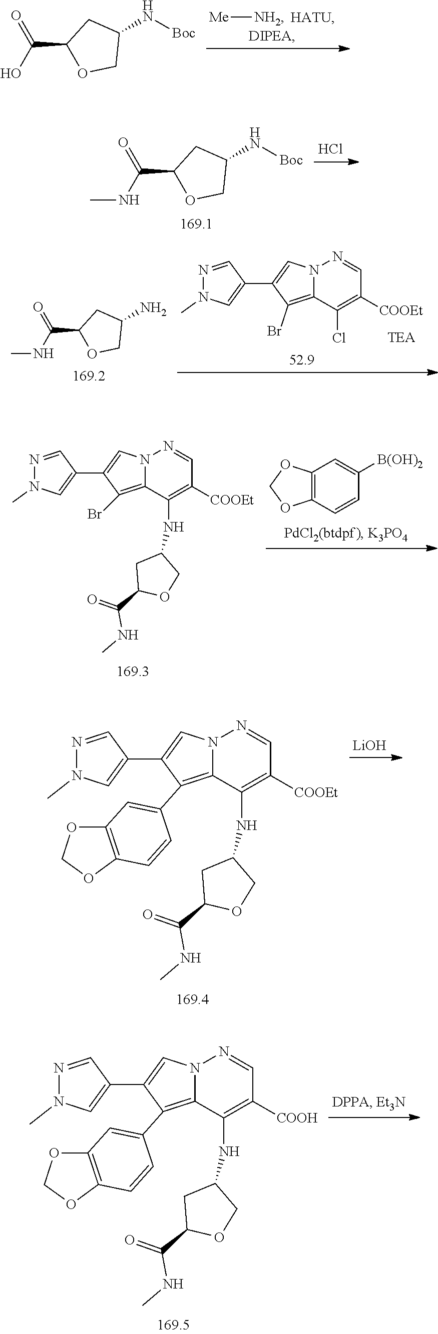

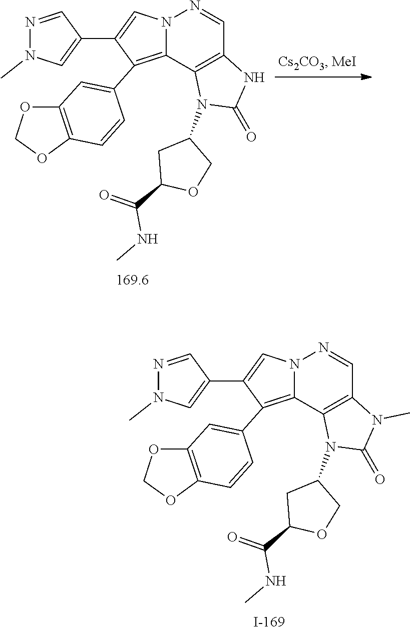

Synthesis of compound 169.1. A solution of (2R,4S)-4-((tert-butoxycarbonyl)amino)tetrahydrofuran-2-carboxylic acid (0.400 g, 1.73 mmol, 1.0 equiv) and HATU (0.987 g, 2.597 mmol, 1.5 equiv) in DMF (8 mL) was stirred at room temperature for 30 min. To the mixture was added methyl amine in THF (0.107 g, 3.46 mmol, 2.0 equiv) and stirred at room temperature for 1 h. It was transferred into ice-water and extracted with ethyl acetate. The combined organic layers were washed with brine, dried over sodium sulfate, filtered and concentrated under reduced pressure. The residue was purified by flash column chromatography on silica gel (3.7% methanol in DCM) to afford 169.1 (0.190 g, 45%). MS(ES): m/z 245.29 [M+H]⁺.

Synthesis of compound 169.2. To a solution of 169.1 (0.190 g, 0.77 mmol, 1.0 equiv) in DCM (6 mL) was added 4 M HCl in dioxane (5 mL) at 0° C. temperature. The reaction was stirred at room temperature for 1 h. It was diluted with DCM and concentrated under reduced pressure to afford 169.2 (0.170 g). It was used in the next step without purification.

Synthesis of compound 169.3. Compound 169.3 was prepared from compound 52.9 and 169.2 following the procedure described in the synthesis of compound 1.9. The product was purified by flash column chromatography on silica gel (Combiflash®, 4.8% methanol in DCM). MS(ES): m/z 492.35 and 494.35 [M+H]⁺.

Synthesis of compound 169.4. Compound 169.4 was prepared from compound 169.3 and benzo[d][1,3]dioxol-5-ylboronic acid following the procedure described in the synthesis of compound 59.12. The product was purified by flash column chromatography on silica gel (50% ethyl acetate in n-hexane). MS (ES+): m/z 533.56 [M+H]⁺.

Synthesis of compound 169.5. Compound 169.5 was prepared from compound 169.4 following the procedure described in the synthesis of compound 1.10. The product was triturated with pentane, dried under reduced pressure and used in the next step without purification. MS (ES+): m/z 505.50 [M+H]⁺.

Synthesis of compound 169.6. Compound 169.6 was prepared from compound 169.5 following the procedure described in the synthesis of compound 1.11. The product was purified by flash column chromatography on silica gel (2.2% methanol in DCM). MS (ES+): m/z 502.50 [M+H]⁺.

Synthesis of compound I-169. Compound I-169 was prepared from compound 169.6 following the procedure described in the synthesis of compound 1.12. The product was purified by flash column chromatography on silica gel (2.0% methanol in DCM). MS (ES): m/z 516.26 [M+H]⁺, 1H-NMR (400 MHz, DMSO-d6): δ 8.41 (s, 1H), 8.12 (s, 1H), 7.66-7.53 (m, 1H), 7.32 (d, J=2.40 Hz, 1H), 7.16 (d, J=0.40 Hz, 1H), 7.02-6.96 (m, 1H), 6.91-6.90 (m, 1H), 6.85-6.83 (m, 1H), 6.19 (d, J=0.80 Hz, 1H), 6.08 (s, 1H), 4.51-4.46 (m, 1H), 4.18-4.09 (m, 1H), 3.95-3.86 (m, 1H), 3.77 (s, 3H), 3.69-3.59 (m, 1H), 3.35 (s, 4H), 2.62 (s, 3H), 1.77-1.66 (m, 1H).

### Example I-170: 3-((1S,3S)-3-(9-(benzo[d][1,3]dioxol-5-yl)-8-(2,4-dimethyloxazol-5-yl)-3-methyl-2-oxo-2,3-dihydro-1H-imidazo[4,5-d]pyrrolo[1,2-b]pyridazin-1-yl)cyclopentyl)oxazolidin-2-one

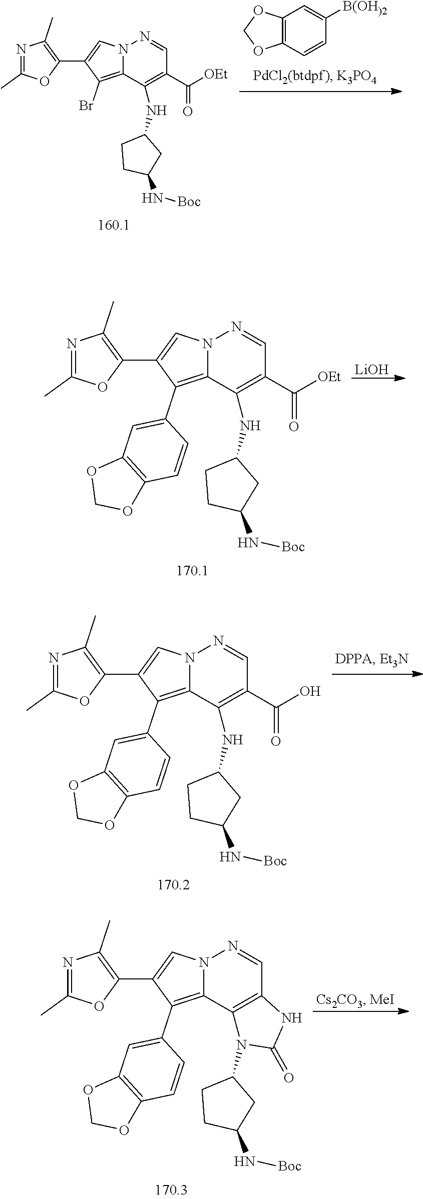

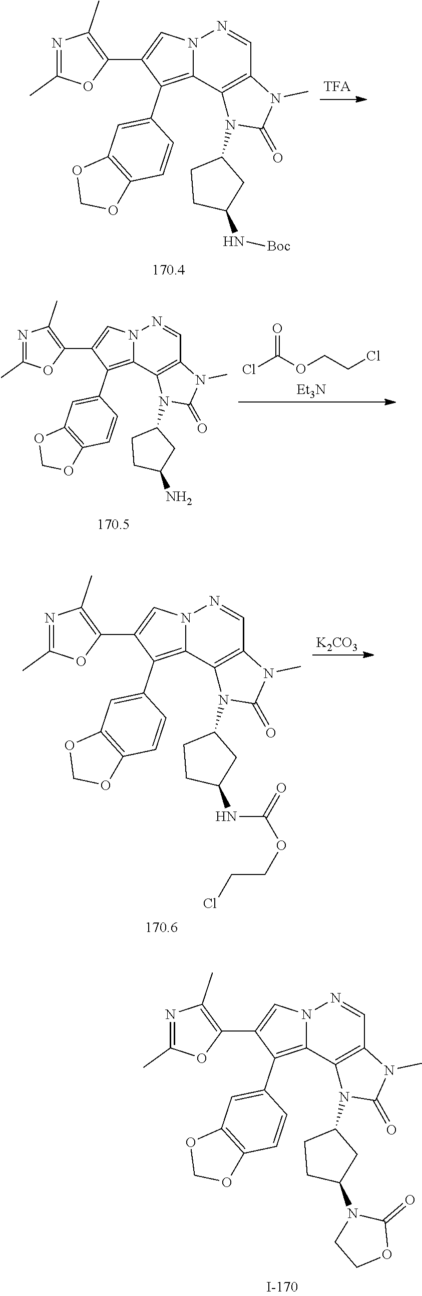

Synthesis of compound 170.1. Compound 170.1 was prepared from compound 160.1 and benzo[d][1,3]dioxol-5-ylboronic acid following the procedure described in the synthesis of compound 59.12. The product was purified by flash column chromatography on silica gel (2.5% methanol in DCM). MS(ES): m/z 604.31 [M+H].

Synthesis of compound 170.2. Compound 170.2 was prepared from compound 170.1 following the procedure described in the synthesis of compound 1.10. The product was triturated with pentane, dried under reduced pressure and used in the next step without purification. MS(ES): m/z 576.5 [M+H]⁺

Synthesis of compound 170.3. Compound 170.3 was prepared from compound 170.2 following the procedure described in the synthesis of compound 1.11. The product was purified by flash column chromatography on silica gel (Combiflash®, 4.1% methanol in DCM). MS(ES): m/z 573.5 [M+H]⁺.

Synthesis of compound 170.4. Compound 170.4 was prepared from compound 170.3 following the procedure described in the synthesis of compound 1.12. The product was purified by flash column chromatography on silica gel (2.7% methanol in DCM). MS(ES): m/z 587.43 [M+H]⁺.

Synthesis of compound 170.5. Compound 170.5 was prepared from compound 170.4 following the procedure described in the synthesis of compound 1.14. The product was triturated with n-pentane and dried under reduced pressure. MS(ES): m/z 487.4 [M+H]⁺. It was used in the next step without purification.

Synthesis of compound 170.6. Compound 170.6 was prepared from compound 170.5 and 2-chloroethyl carbonochloridate following the procedure described in the synthesis of compound 9.7. The product was used in the next step without purification. MS(ES): m/z 594.3 [M+H]⁺.

Synthesis of compound I-170. Compound I-164 was prepared from compound 164.6 following the procedure described in the synthesis of compound I-9. The product was purified by flash column chromatography on silica gel (Combiflash®, 2.9% methanol in DCM). MS (ES): m/z 557.33 [M+H]⁺; 1H-NMR (400 MHz, DMSO-d6): δ 8.53 (s, 1H), 8.11 (s, 1H), 7.08-6.85 (m, 3H), 6.14-6.05 (m, 2H), 4.39-4.26 (m, 3H), 3.95-3.83 (m, 1H), 3.38 (s, 3H), 3.33-3.29 (m, 2H), 2.23 (s, 3H), 2.12-2.08 (m, 2H), 1.94-1.93 (m, 3H), 1.79 (m, 1H), 1.57-1.54 (m, 1H), 1.47-1.40 (m, 1H), 1.072-1.00 (m, 1H).

### Example I-171: 3-((1S,3S)-3-(9-(2,3-dihydrobenzo[b][1,4]dioxin-6-yl)-8-(2,4-dimethyloxazol-5-yl)-3-methyl-2-oxo-2,3-dihydro-1H-imidazo[4, 5-d]pyrrolo[1,2-b]pyridazin-1-yl)cyclopentyl)oxazolidin-2-one

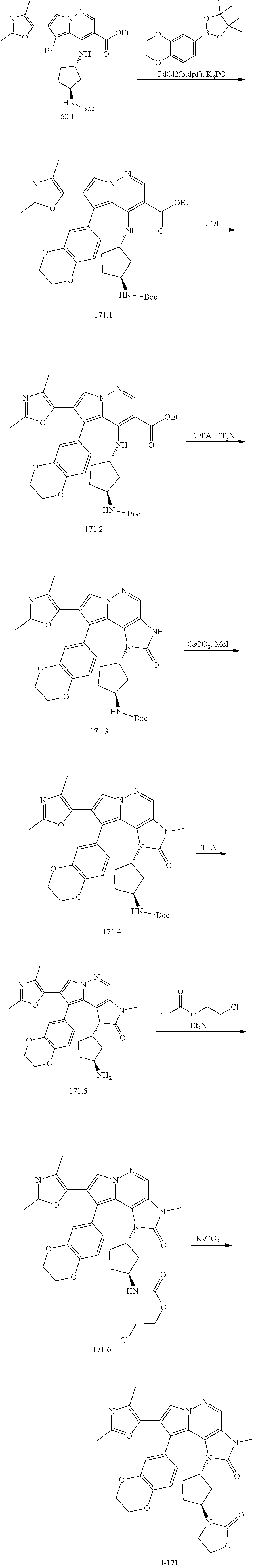

Synthesis of compound 171.1. Compound 171.1 was prepared from compound 160.1 and 2-(2,3-dihydrobenzo[b][1,4]dioxin-6-yl)-4,4,5,5-tetramethyl-1,3,2-dioxaborolane following the procedure described in the synthesis of compound 59.12. The product was purified by flash column chromatography on silica gel (1.4% methanol in DCM). MS(ES): m/z 618.5 [M+H]⁺.

Synthesis of compound 171.2. Compound 171.2 was prepared from compound 171.1 following the procedure described in the synthesis of compound 1.10. The product was used in the next step without purification. MS(ES): m/z 590.4 [M+H]⁺.

Synthesis of compound 171.3. Compound 171.3 was prepared from compound 171.2 following the procedure described in the synthesis of compound 1.11. The product was purified by flash column chromatography on silica gel (Combiflash®, 2.5% methanol in DCM). MS(ES): m/z 587.5 [M+H]⁺.

Synthesis of compound 171.4. Compound 171.4 was prepared from compound 171.3 following the procedure described in the synthesis of compound 1.12. The product was purified by flash column chromatography on silica gel (1.5% methanol in DCM). MS(ES): m/z 601.2 [M+H]⁺.

Synthesis of compound 171.5. Compound 171.5 was prepared from compound 171.4 following the procedure described in the synthesis of compound 1.14. The product was used in the next step without purification. MS(ES) m/z 501.8 [M+H]⁺.

Synthesis of compound 171.6. Compound 171.6 was prepared from compound 171.5 and 2-chloroethyl carbonochloridate following the procedure described in the synthesis of compound 9.7. The product was purified by flash column chromatography on silica gel (Combiflash®, 2.3% methanol in DCM). MS(ES): 594.2 m/z [M+H]⁺.

Synthesis of I-171. Compound I-171 was prepared from compound 171.6 following the procedure described in the synthesis of compound I-9. The product was purified by flash column chromatography on silica gel (Combiflash®, 3.6% methanol in DCM). MS(ES) m/z 571.3 m/z [M+H]⁺; 1H-NMR (400 MHz, DMSO-d6): δ 8.56 (s, 1H), 8.09 (s, 1H), 6.99-6.83 (m, 3H), 4.34-4.24 (m, 8H), 3.29-3.22 (m, 3H), 2.68-2.67 (m, 2H), 2.30 (s, 3H), 2.15-2.13 (m, 2H), 1.96-1.96 (m, 3H), 1.89-1.86 (m, 1H), 1.57-1.55 (m, 1H), 1.44-1.39 (m, 1H), 1.06-0.95 (m, 1H).

### Example I-172: (1R,2R)—N-((1s,4S)-4-(9-(3,4-dihydro-1H-pyrrolo[2,1-c][1,4]oxazin-7-yl)-3-methyl-8-(4-methylthiazol-2-yl)-2-oxo-2,3-dihydro-1H-imidazo[4,5-d]pyrrolo[1,2-b]pyridazin-1-yl)-1-methylcyclohexyl)-2-fluorocyclopropane-1-carboxamide

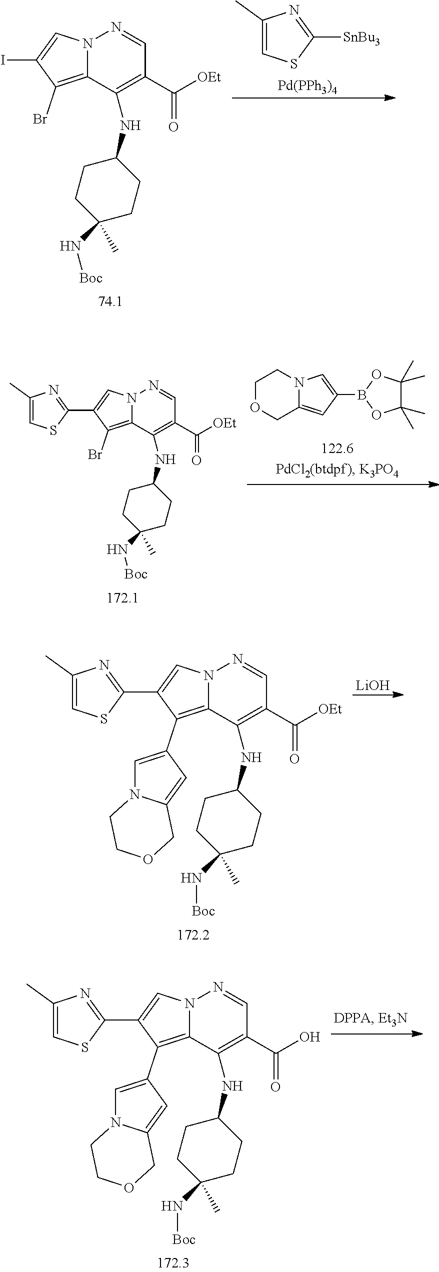

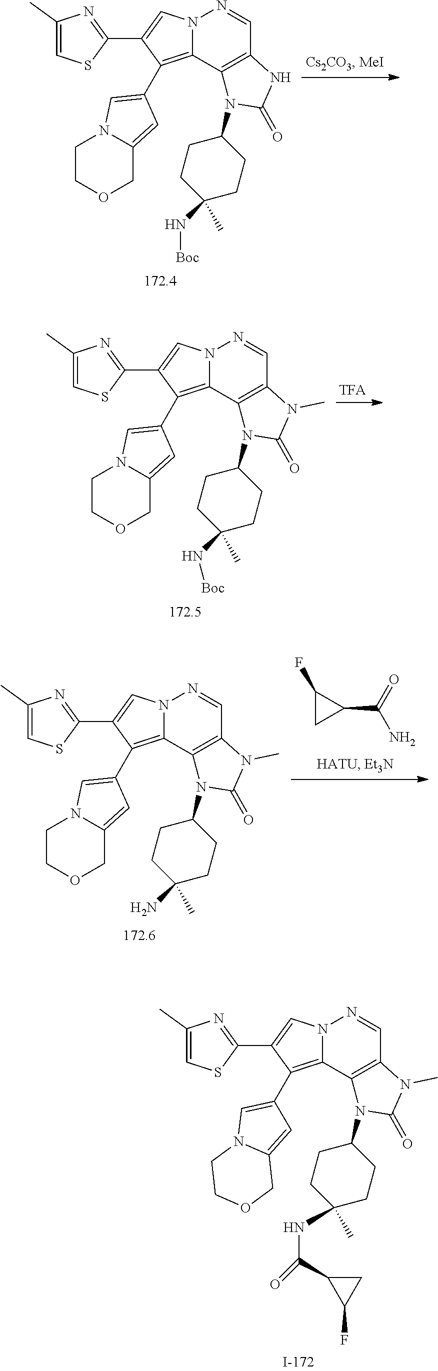

Synthesis of compound 172.1. A solution of 74.1 (1.0 g, 1.61 mmol, 1.0 equiv) and 4-methyl-2-(tributylstannyl)thiazole (0.937 g, 2.41 mmol, 1.5 equiv) in 1,4-dioxane (5 mL) was degassed by bubbling through a stream of argon for 10 min.

Tetrakis(triphenylphosphine)palladium (0.185 g, 0.160 mmol, 0.1 equiv) was added to the reaction mixture and degassed for 10 min. The mixture was stirred at 120° C. for 30 min. It cooled to rt and concentrated under reduced pressure. The residue was purified by flash column chromatography on silica gel (Combiflash®, 15% ethyl acetate in n-hexane) to afford 172.1 (0.450 g, 47%). MS(ES): m/z 592.6 and 594.5 [M+H]⁺.

Synthesis of compound 172.2. Compound 172.2 was prepared from compound 172.1 and 122.6 following the procedure described in the synthesis of compound 59.12. The product was purified by flash column chromatography on silica gel (Combiflash®, 55% ethyl acetate in n-hexane). MS(ES): m/z 635.9 [M+H]⁺.

Synthesis of compound 172.3. Compound 162.3 was prepared from compound 162.2 following the procedure described in the synthesis of compound 1.10. The product was triturated with pentane, dried under reduced pressure and used in the next step without purification. MS(ES): m/z 607.8 [M+H]⁺.

Synthesis of compound 172.4. Compound 172.4 was prepared from compound 172.3 following the procedure described in the synthesis of compound 1.11. The product was purified by flash column chromatography on silica gel (Combiflash®, 2.2% methanol in DCM). MS(ES): m/z 604.7 [M+H]⁺.

Synthesis of compound 172.5. Compound 172.5 was prepared from compound 172.4 following the procedure described in the synthesis of compound 1.12. The product was purified by flash column chromatography on silica gel (1.8% methanol in DCM). MS(ES): m/z 618.9 [M+H]⁺.

Synthesis of compound 172.6. Compound 172.6 was prepared from compound 172.5 following the procedure described in the synthesis of compound 1.14. The product was triturated with pentane and dried under reduced pressure. MS(ES): m/z 518.8 [M+H]⁺. It was used in next step without purification.

Synthesis of compound I-172. Compound I-172 was prepared from compound 172.6 and (1R,2R)-2-fluorocyclopropane-1-carboxylic acid following the procedure described in the synthesis of compound I-45. The product was purified by flash column chromatography on silica gel (2.2% methanol in DCM). MS(ES): m/z 604.31 [M+H]⁺, 1H-NMR (400 MHz, DMSO-d6): δ 8.44 (s, 1H), 8.27 (s, 1H), 7.51 (s, 1H), 7.05 (s, 1H), 6.85 (s, 1H), 5.84 (s, 1H), 4.89-4.82 (m, 2H), 4.08-4.01 (m, 6H), 3.42 (s, 3H), 2.39-2.31 (m, 4H), 2.28-2.15 (m, 2H), 1.89-1.86 (m, 1H), 1.50-1.45 (m, 1H), 1.26-1.23 (m, 6H), 0.98-0.93 (m, 1H), 0.86-0.82 (m, 1H), 0.59 (m, 1H).

### Example I-173: 3-((1S,3S)-3-(9-(4,7-dihydro-5H-thieno[2,3-c]pyran-2-yl)-8-(2,4-dimethyloxazol-5-yl)-3-methyl-2-oxo-2,3-dihydro-1H-imidazo[4,5-d]pyrrolo[1,2-b]pyridazin-1-yl)cyclopentyl)oxazolidin-2-one

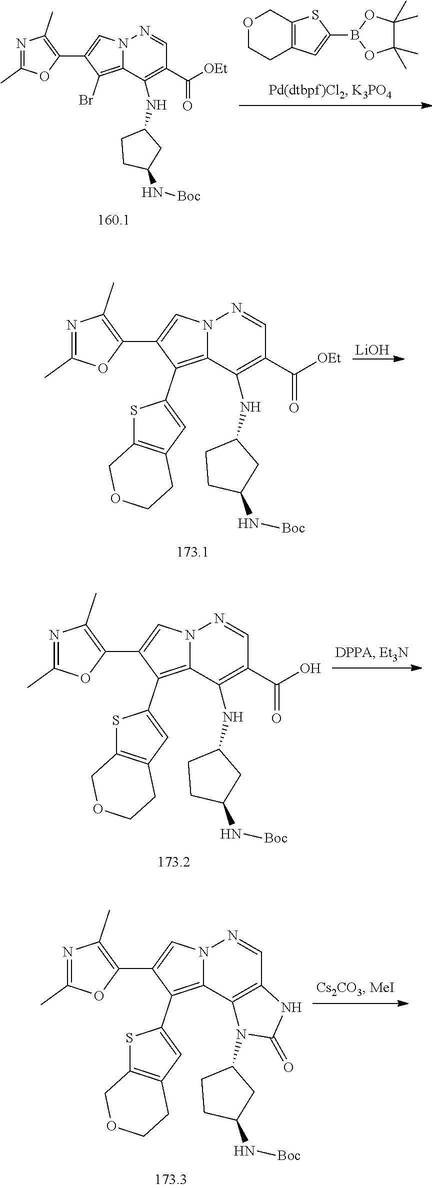

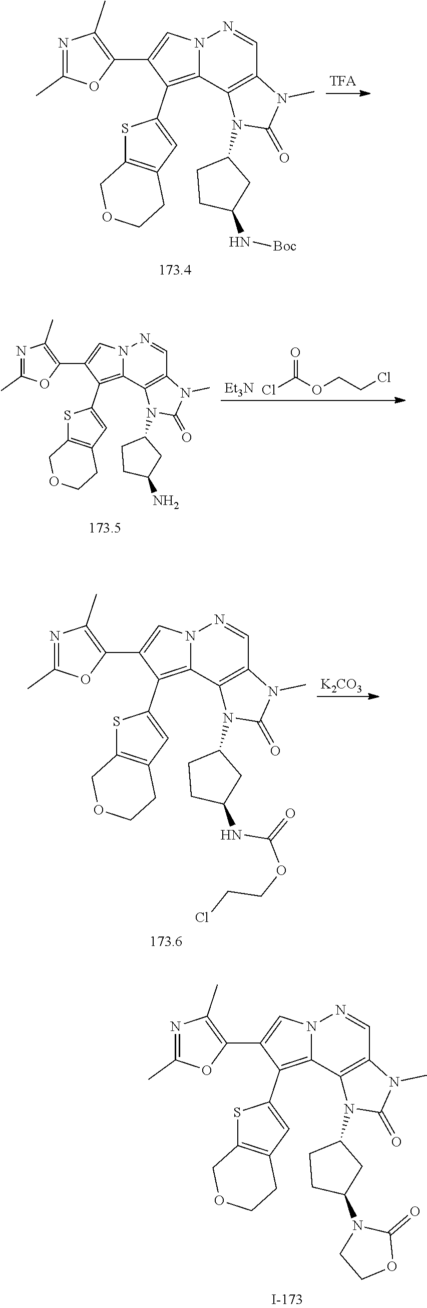

Synthesis of compound 173.1. Compound 173.1 was prepared from compound 160.1 and 2-(4,7-dihydro-5H-thieno[2,3-c]pyran-2-yl)-4,4,5,5-tetramethyl-1,3,2-dioxaborolane following the procedure described in the synthesis of compound 59.12. The product was purified by flash column chromatography on silica gel (1.5% methanol in DCM). MS(ES): m/z 622.6 [M+H]⁺.

Synthesis of compound 173.2. Compound 173.2 was prepared from compound 173.1 following the procedure described in the synthesis of compound 1.10. The product was triturated with pentane, dried under reduced pressure and used in the next step without purification. MS(ES): m/z 594.8 [M+H]⁺.

Synthesis of compound 173.3. Compound 173.3 was prepared from compound 173.2 following the procedure described in the synthesis of compound 1.11. The product was purified by flash column chromatography on silica gel (Combiflash®, 2.3% methanol in DCM). MS(ES): m/z 591.4 [M+H]⁺.

Synthesis of compound 173.4. Compound 173.4 was prepared from compound 173.3 following the procedure described in the synthesis of compound 1.12. The product was purified by flash column chromatography on silica gel (1.7% methanol in DCM). MS(ES): m/z 605.5 [M+H]⁺.

Synthesis of compound 173.5. Compound 173.5 was prepared from compound 173.4 following the procedure described in the synthesis of compound 1.14. MS(ES) m/z 505.4 [M+H]⁺. It was used in the next step without further purification.

Synthesis of compound 173.6. Compound 173.6 was prepared from compound 173.5 and 2-chloroethyl carbonochloridate following the procedure described in the synthesis of compound 9.7. The product was purified by flash column chromatography on silica gel (Combiflash®, 3.3% methanol in DCM). MS(ES): 612.6 m/z [M+H]⁺.

Synthesis of I-173. Compound I-173 was prepared from compound 173.6 following the procedure described in the synthesis of compound I-9. The product was purified by flash column chromatography on silica gel (Combiflash®, 3.8% methanol in DCM). MS(ES) m/z 575.2 m/z [M+H]⁺; 1H-NMR (400 MHz, DMSO-d6): δ 8.57 (s, 1H), 8.14 (s, 1H), 6.95 (s, 1H), 4.75-4.73 (m, 2H), 4.44-4.39 (m, 1H), 4.29-4.24 (m, 2H), 3.90 (s, 3H), 3.41-3.34 (m, 5H), 2.70-2.67 (m, 2H), 2.27 (s, 3H), 2.15-2.13 (m, 2H), 2.02 (m, 3H), 1.85-1.82 (m, 1H), 1.69-1.66 (m, 1H), 1.56-1.54 (m, 1H), 1.22-1.19 (m, 1H).

### Example I-174: 3-((1S,3S)-3-(9-(2,3-dihydrobenzofuran-6-yl)-8-(2,4-dimethyloxazol-5-yl)-3-methyl-2-oxo-2,3-dihydro-1H-imidazo[4,5-d]pyrrolo[1,2-b]pyridazin-1-yl)cyclopentyl)oxazolidin-2-one

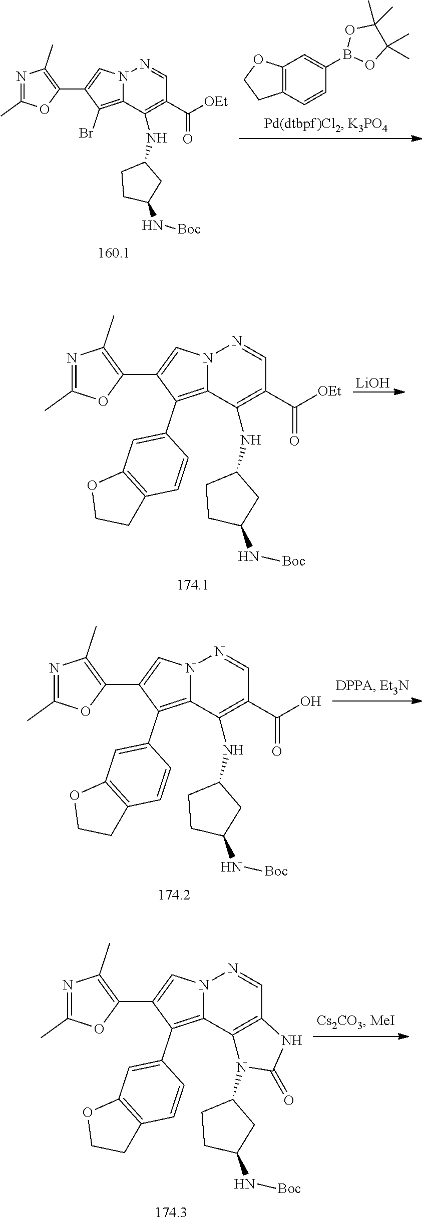

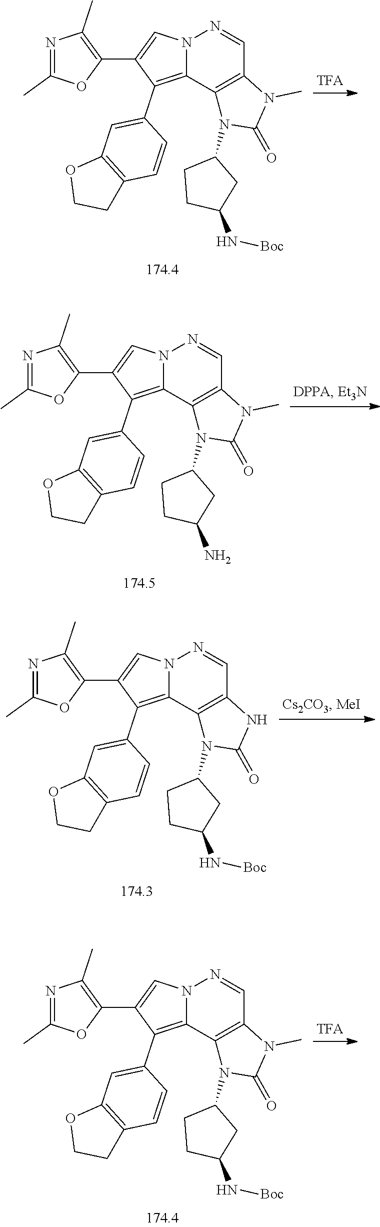

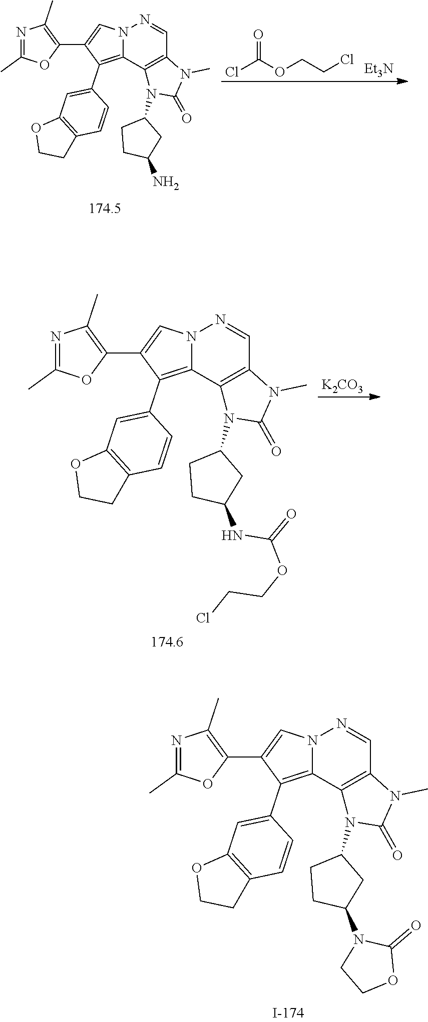

Synthesis of compound 174.1. Compound 1741 was prepared from compound 1601 and 2-(2,3-dihydrobenzofuran-6-yl)-4,4,5,5-tetramethyl-1,3,2-dioxaborolane following the procedure described in the synthesis of compound 59.12. The product was purified by flash column chromatography on silica gel (2.5% methanol in DCM). MS(ES): m/z 602.41 [M+H]⁺.

Synthesis of compound 174.2. Compound 174.2 was prepared from compound 174.1 following the procedure described in the synthesis of compound 1.10. The product was triturated with pentane. MS(ES): m/z 574.2 [M+H]⁺.

Synthesis of compound 174.3. Compound 174.3 was prepared from compound 174.2 following the procedure described in the synthesis of compound 1.11. The product was purified by flash column chromatography on silica gel (Combiflash®, 4.0% methanol in DCM). MS(ES): m/z 571.3 [M+H]⁺.

Synthesis of compound 174.4. Compound 174.4 was prepared from compound 174.3 following the procedure described in the synthesis of compound 1.12. The product was purified by flash column chromatography on silica gel (3.3% methanol in DCM). MS(ES): m/z 585.5 [M+H]⁺.

Synthesis of compound 174.5. Compound 174.5 was prepared from compound 174.4 following the procedure described in the synthesis of compound 1.14. The product was triturated with n-pentane and dried under reduced pressure. MS(ES): m/z 485.3 [M+H]⁺. It was used in next step without further purification.

Synthesis of compound 174.6. Compound 174.6 was prepared from compound 174.5 and 2-chloroethyl carbonochloridate following the procedure described in the synthesis of compound 9.7. The product was used in the next step without purification. MS(ES): m/z 592.25 [M+H]⁺.

Synthesis of compound I-174. Compound I-174 was prepared from compound 174.6 following the procedure described in the synthesis of compound I-9. The product was purified by flash column chromatography on silica gel (Combiflash®, 2.8% methanol in DCM). MS (ES): m/z 555.28 [M+H]⁺; 1H-NMR (400 MHz, DMSO-d6): δ 8.52 (s, 1H), 8.10 (s, 1H), 7.35-7.30 (m, 1H), 6.84-6.74 (m, 2H), 4.58-4.56 (m, 2H), 4.29-4.27 (m, 3H), 3.79 (m, 1H), 3.40 (s, 3H), 3.31-3.26 (m, 5H), 2.23 (s, 3H), 2.07 (m, 2H), 1.88 (m, 2H), 1.75 (m, 1H), 1.53 (m, 1H), 1.40-1.35 (m, 1H), 0.95 (m, 1H).

### Example I-175: (1R,2R)-2-fluoro-N-((1s,4S)-1-methyl-4-(3-methyl-9-(1-methyl-1H-indazol-5-yl)-8-(oxazol-5-yl)-2-oxo-2,3-dihydro-1H-imidazo[4,5-d]pyrrolo[1,2-b]pyridazin-1-yl)cyclohexyl)cyclopropane-1-carboxamide

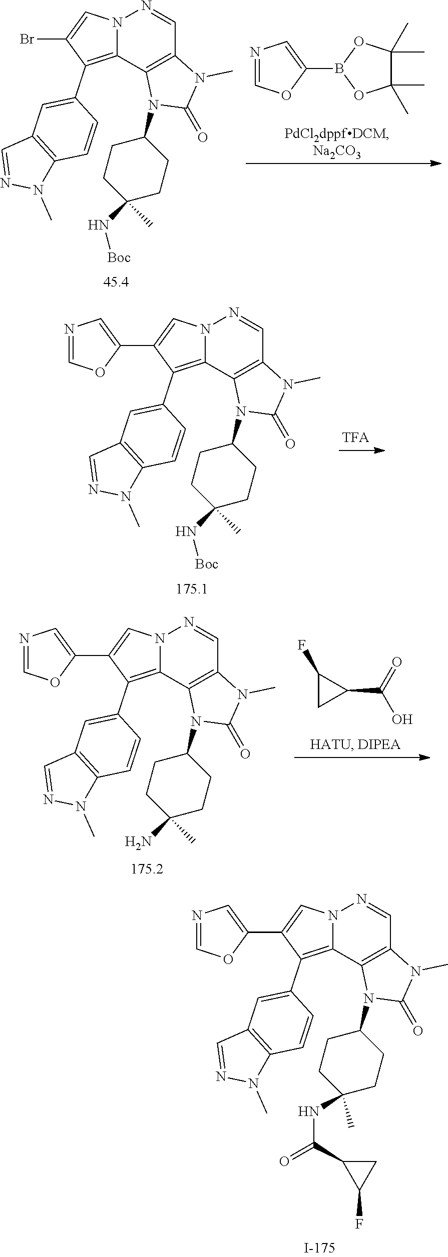

Synthesis of compound 175.1. Compound 175.1 was prepared from compound 45.4 and 5-(4,4,5,5-tetramethyl-1,3,2-dioxaborolan-2-yl)oxazole following the procedure described in the synthesis of compound 1.13. The product was purified by flash column chromatography on silica gel (Combiflash®, 3.5% methanol in DCM). MS(ES): m/z 497.4 [M+H]⁺.

Synthesis of compound 175.2. Compound 175.2 was prepared from compound 175.1 following the procedure described in the synthesis of compound 1.14. The product was triturated with pentane and dried under reduced pressure. It was used in the next step without purification. MS(ES): 497.2 m/z [M+H]⁺.

Synthesis of I-175. Compound I-175 was prepared from compound 175.2 and (1R,2R)-2-fluorocyclopropane-1-carboxylic acid following the procedure described in the synthesis of compound I-45. The product was purified by flash column chromatography on silica gel (3.7% methanol in DCM). MS(ES): m/z 583.33 [M+H]⁺; 1H-NMR (400 MHz, DMSO-d6): δ 8.52 (d, J=4.00 Hz, 1H), 8.27 (d, J=2.80 Hz, 2H), 8.15 (s, 1H), 7.89-7.85 (m, 2H), 7.43 (dd, J=0.80, 8.60 Hz, 1H), 7.31 (s, 1H), 5.83 (s, 1H), 4.78-4.61 (m, 1H), 4.15 (s, 3H), 3.39-3.34 (m, 3H), 3.20-3.14 (m, 1H), 2.40-2.26 (m, 2H), 1.87-1.68 (m, 3H), 1.42-1.25 (m, 2H), 1.02-0.88 (m, 3H), 0.66 (s, 3H), −0.05-0.08 (m, 1H).

### Example I-176: 3-((1S,3S)-3-(9-(3,4-dihydro-1H-pyrrolo[2,1-c][1,4]oxazin-7-yl)-3-methyl-2-oxo-8-(thiazol-5-yl)-2,3-dihydro-1H-imidazo[4,5-d]pyrrolo[1,2-b]pyridazin-1-yl)cyclopentyl)oxazolidin-2-one

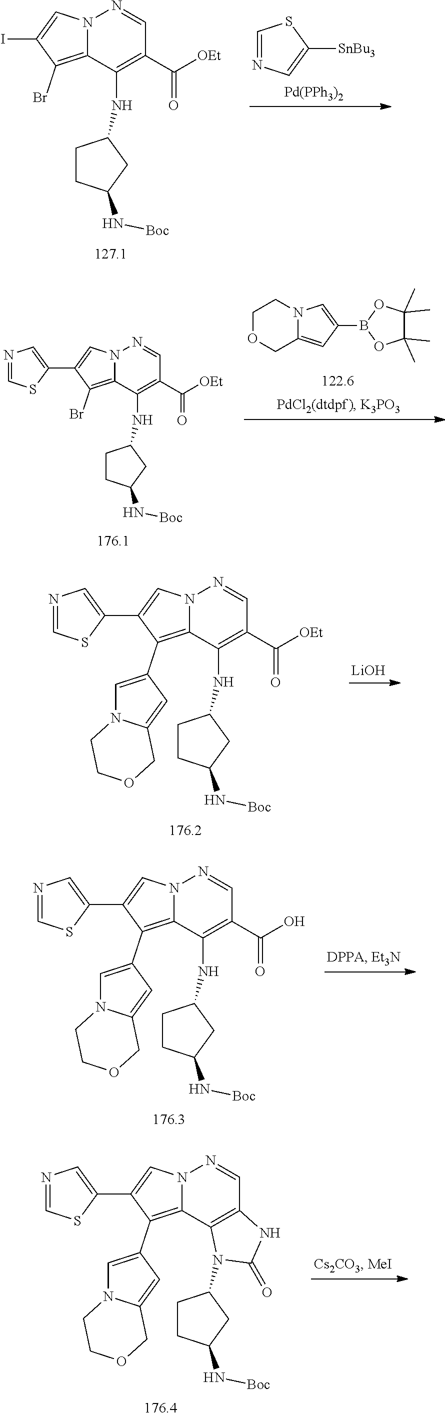

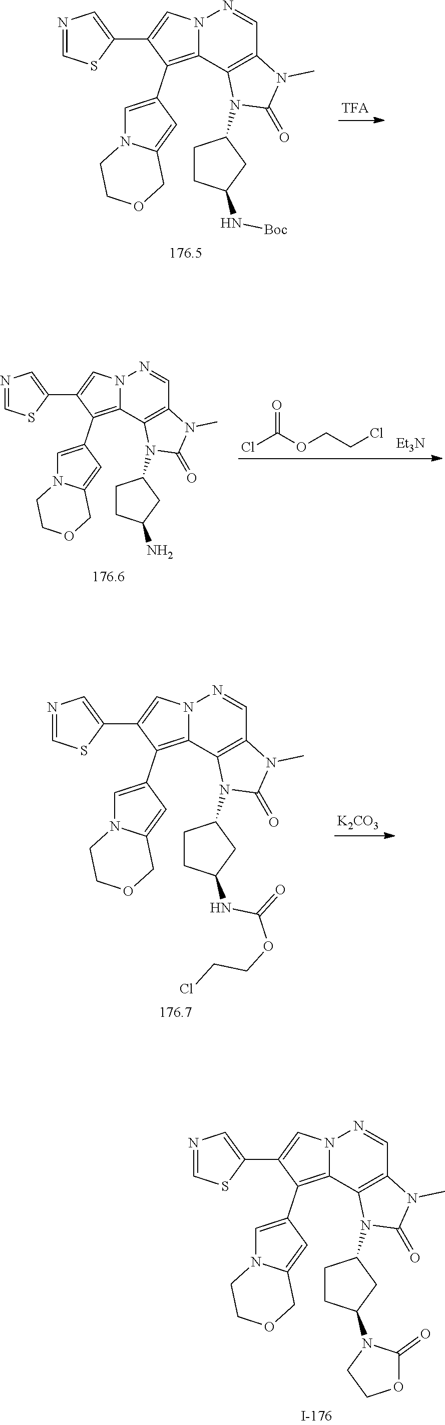

Synthesis of compound 176.1. Compound 176.1 was prepared from compound 127.1 and 5-(tributylstannyl)thiazole following the procedure described in the synthesis of compound 172.1. The product was purified by flash column chromatography on silica gel (Combiflash®, 25% ethyl acetate in n-hexane). MS(ES): m/z 550.2 and 552.1 [M+H]⁺.

Synthesis of compound 176.2. Compound 176.2 was prepared from compound 176.1 and 12.6 following the procedure described in the synthesis of compound 59.12. The product was purified by flash column chromatography on silica gel (Combiflash®, 50% ethyl acetate in n-hexane). MS(ES): m/z 593.2 [M+H]⁺.

Synthesis of compound 176.3. Compound 176.3 was prepared from compound 176.2 following the procedure described in the synthesis of compound 1.10. It was used in the next step without purification. MS (ES+): m/z 565.3 [M+H]⁺.

Synthesis of compound 176.4. Compound 176.4 was prepared from compound 176.3 following the procedure described in the synthesis of compound 1.11. The product was purified by flash column chromatography on silica gel (90% ethyl acetate in hexane). MS (ES+): m/z 562.3 [M+H]⁺.

Synthesis of compound 176.5. Compound 176.5 was prepared from compound 176.4 following the procedure described in the synthesis of compound 1.12. It was used in the next step without purification. MS (ES+): m/z 576.3 [M+H]⁺.

Synthesis of compound 176.6. Compound 176.6 was prepared from compound 176.5 following the procedure described in the synthesis of compound 1.14. The product was used in the next step without purification. MS (ES+): m/z 476.2 [M+H]⁺.

Synthesis of compound 176.7. Compound 176.7 was prepared from compound 176.6 and 2-chloroethyl carbonochloridate following the procedure described in the synthesis of compound 9.7. The product was used in the next step without purification. MS(ES) m/z 583.2 [M+H]⁺.

Synthesis of I-176. Compound I-176 was prepared from compound 176.7 following the procedure described in the synthesis of compound I-9. The product was purified by flash column chromatography on silica gel (Combiflash®, 2.5% methanol in DCM). MS(ES) m/z 546.3. 1H-NMR (400 MHz, DMSO-d6): δ 8.87 (s, 1H), 8.45 (s, 1H), 8.39 (s, 1H), 8.05 (s, 1H), 6.86 (s, 1H), 5.90 (s, 1H), 4.86-4.78 (m, 2H), 4.4-438 (m, 2H), 4.30-4.25 (m, 2H), 4.07-3.95 (m, 3H), 3.48-3.43 (m, 2H), 3.39 (s, 3H), 2.06-2.00 (m, 2H), 1.88-1.80 (m, 1H), 1.73-1.58 (m, 2H), 1.56-1.42 (m, 1H), 1.39-1.32 (m, 1H).

### Example I-177: 3-((1S,3S)-3-(8-(2,4-dimethyloxazol-5-yl)-3-methyl-2-oxo-9-(pyrazolo[1,5-a]pyridin-5-yl)-2,3-dihydro-1H-imidazo[4,5-d]pyrrolo[1,2-b]pyridazin-1-yl)cyclopentyl)oxazolidin-2-one

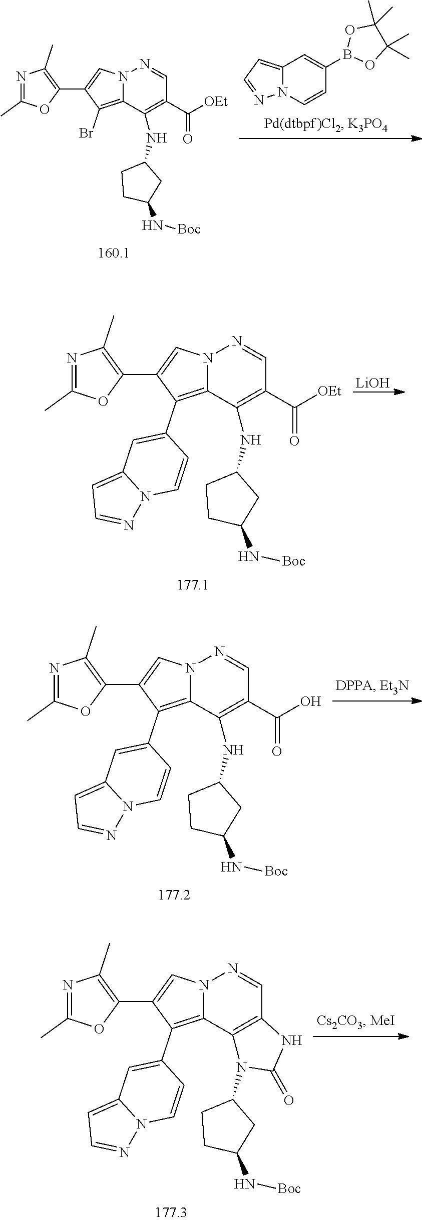

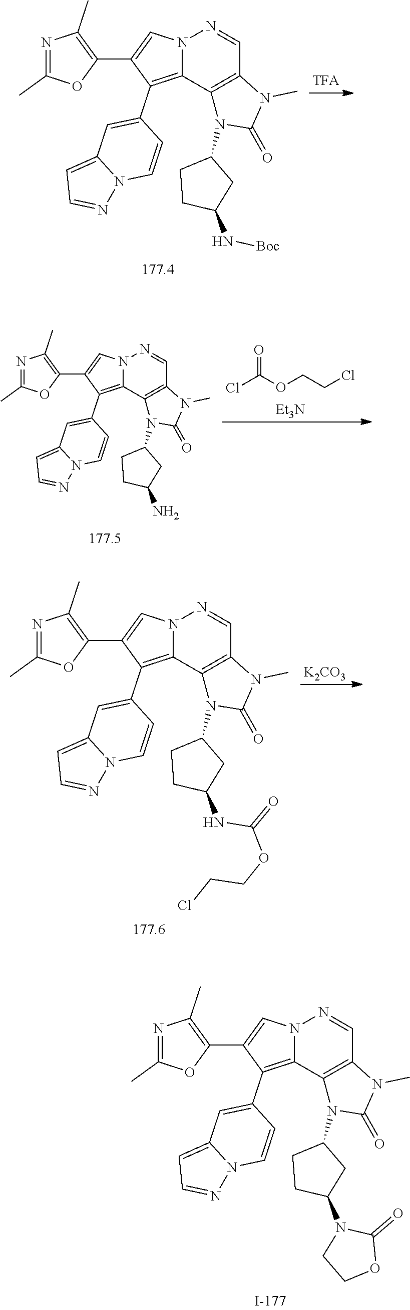

Synthesis of compound 177.1. Compound 177.1 was prepared from compound 160.1 and 5-(4,4,5,5-tetramethyl-1,3,2-dioxaborolan-2-yl)pyrazolo[1,5-a]pyridine following the procedure described in the synthesis of compound 59.12. The product was purified by flash column chromatography on silica gel (45% ethyl acetate in n-hexane). MS (ES+): m/z 600.7 [M+H]⁺.

Synthesis of compound 177.2. Compound 177.2 was prepared from compound 177.1 following the procedure described in the synthesis of compound 1.10. The product was used in the next step without purification. MS (ES+): m/z 572.6 [M+H]⁺.

Synthesis of compound 177.3. Compound 177.3 was prepared from compound 177.2 following the procedure described in the synthesis of compound 1.11. The product was purified by flash column chromatography on silica gel (3% methanol in DCM). MS (ES+): m/z 569.7 [M+H]⁺.

Synthesis of compound 177.4. Compound 177.4 was prepared from compound 177.3 following the procedure described in the synthesis of compound 1.12. The product was purified by flash column chromatography on silica gel (2.9% methanol in DCM). MS (ES+): m/z 583.7 [M+H]⁺.

Synthesis of compound 177.5. Compound 177.5 was prepared from compound 177.4 following the procedure described in the synthesis of compound 1.14. The product was used in the next step without purification. MS (ES+): m/z 483.5 [M+H]⁺.

Synthesis of compound 177.6. Compound 177.6 was prepared from compound 177.5 and 2-chloroethyl carbonochloridate following the procedure described in the synthesis of compound 9.7. The product was purified by flash column chromatography on silica gel (Combiflash®, 2.6% methanol in DCM). MS (ES+): m/z 590.05 [M+H]⁺.

Synthesis of compound I-177. Compound I-177 was prepared from compound 177.6 following the procedure described in the synthesis of compound I-9. The product was purified by flash column chromatography on silica gel (2.6% methanol in DCM). MS (ES+): m/z 553.3 [M+H]⁺. ¹H NMR (400 MHz, DMSO-d₆): δ 8.82 (s, 1H), 8.59 (s, 1H), 8.21 (s, 1H), 8.08 (s, 1H), 7.76 (d, J=19.20 Hz, 1H), 6.91 (s, 1H), 6.66 (s, 1H), 4.21 (s, 2H), 4.12-4.06 (m, 2H), 3.42 (s, 3H), 3.06 (s, 1H), 2.88 (d, J=7.20 Hz, 1H), 2.16 (s, 4H), 2.01 (s, 3H), 1.35 (s, 2H), 1.24 (d, J=3.60 Hz, 3H).

### Example I-178: (1R,2R)—N-((1s,4S)-4-(9-(3,4-dihydro-1H-pyrrolo[2,1-c][1,4]oxazin-7-yl)-3-methyl-2-oxo-8-(thiazol-5-yl)-2,3-dihydro-1H-imidazo[4,5-d]pyrrolo[1,2-b]pyridazin-1-yl)-1-methylcyclohexyl)-2-fluorocyclopropane-1-carboxamide

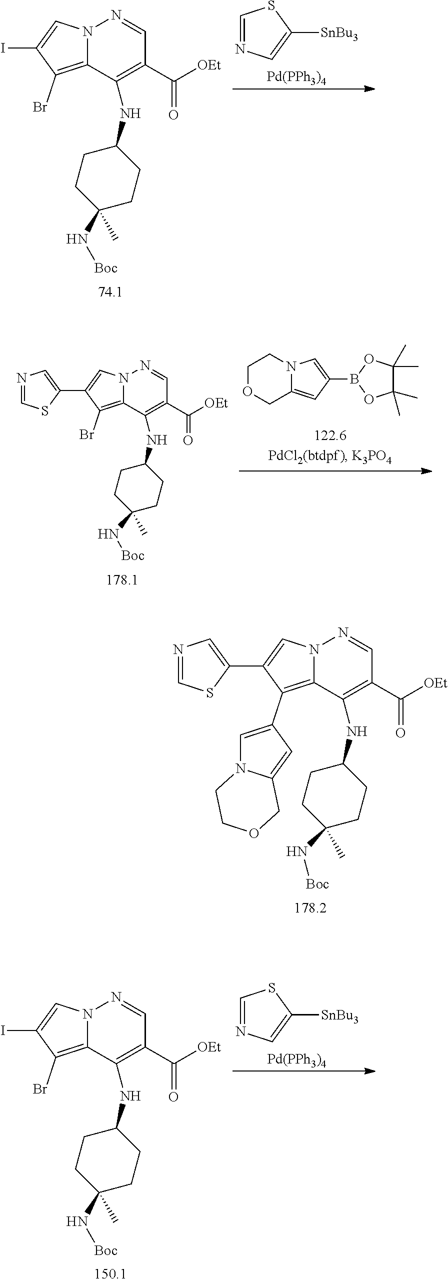

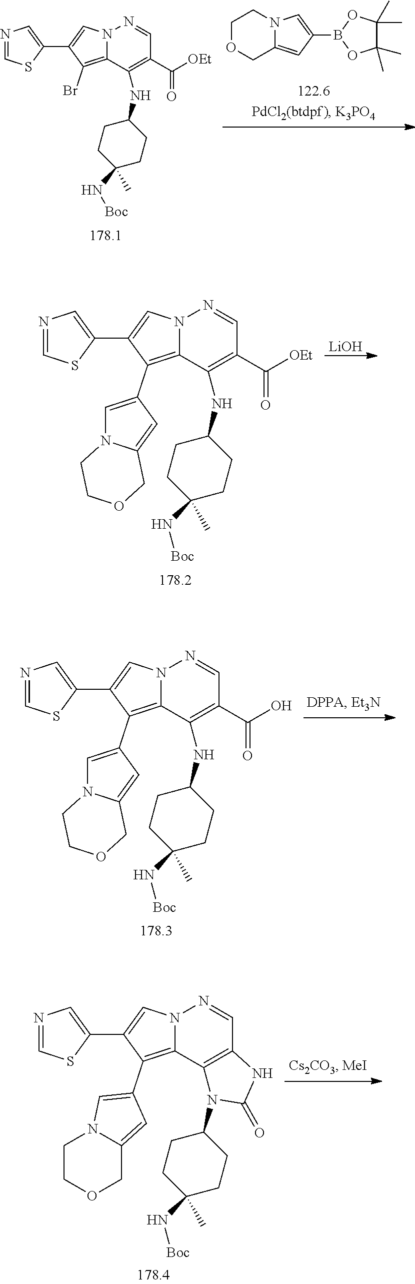

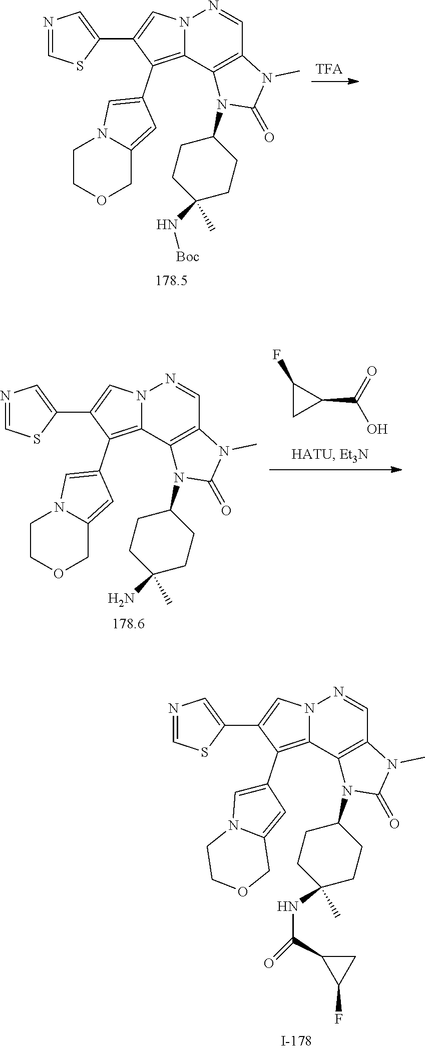

Synthesis of compound 178.1. Compound 178.1 was prepared from compound 74.1 and 5-(tributylstannyl)thiazole following the procedure described in the synthesis of compound 172.1 The product was purified by flash column chromatography on silical gel(Combiflash®, 51% ethyl acetate is hexane). MS(ES): m/z 578.14 and 580.14 [M+H]⁺.

Synthesis of compound 178.2. Compound 178.2 was prepared from compound 178.1 and 122.6 following the procedure described in the synthesis of compound 59.12. The product was purified by flash column chromatography on silical gel (55% ethyl acetate in hexane). MS(ES): m/z 621.77 [M+H]⁺.

Synthesis of compound 178.3. Compound 178.3 was prepared from compound 178.2 following the procedure described in the synthesis of compound 1.10. The product was used in the next step without purification. MS(ES): m/z 593.71 [M+H]⁺.

Synthesis of compound 178.4. Compound 178.4 was prepared from compound 178.3 following the procedure described in the synthesis of compound 1.11. The product was purified by flash column chromatography on silica gel (Combiflash®, 2.1% methanol in DCM). MS(ES): m/z 590.5 [M+H]⁺.

Synthesis of compound 178.5. Compound 178.5 was prepared from compound 178.4 following the procedure described in the synthesis of compound 1.12. The product was purified by flash column chromatography on silica gel (1.8% methanol in DCM). MS(ES): m/z 604.6 [M+H]⁺.

Synthesis of compound 178.6. Compound 178.6 was prepared from compound 178.5 following the procedure described in the synthesis of compound 1.14. The product was used in the next step without purification. MS(ES): m/z 504.62 [M+H]⁺.

Synthesis of compound I-178. Compound I-178 was prepared from compound 178.6 and (1R,2R)-2-fluorocyclopropane-1-carboxylic acid following the procedure described in the synthesis of compound I-45. The product was purified by flash column chromatography on silica gel (Combiflash®, 3.2% methanol in DCM). MS (ES): m/z 590.3 [M+H]⁺; ¹H-NMR (400 MHz, DMSO-d6): δ 8.86 (s, 1H), 8.40 (d, J=10.00 Hz, 2H), 8.01 (s, 1H), 7.50 (s, 1H), 6.80 (s, 1H), 5.84 (s, 1H), 4.88-4.68 (m, 3H), 4.08-4.03 (m, 5H), 3.39 (s, 3H), 2.34-2.19 (m, 2H), 1.89-1.86 (m, 1H), 1.50-1.45 (m, 1H), 1.45-1.43 (m, 1H), 1.27 (d, J=6.00 Hz, 3H), 1.24 (d, J=3.20 Hz, 3H), 0.98-0.96 (m, 1H), 0.86-0.84 (m, 1H), 0.81-0.58 (m, 1H).

### Example I-179-a and I-179-b: ((1S,2S)-2-(9-(benzo[d][1,3]dioxol-5-yl)-3-methyl-8-(1-methyl-1H-pyrazol-4-yl)-2-oxo-2,3-dihydro-1H-imidazo[4,5-d]pyrrolo[1,2-b]pyridazin-1-yl)-N-((1s,4R)-4-methoxycyclohexyl)cyclopropane-1-carboxamide and (1R,2R)-2-(9-(benzo[d][1,3]dioxol-5-yl)-3-methyl-8-(1-methyl-1H-pyrazol-4-yl)-2-oxo-2,3-dihydro-1H-imidazo[4, 5-d]pyrrolo[1,2-b]pyridazin-1-yl)-N-((1s,4S)-4-methoxycyclohexyl)cyclopropane-1-carboxamide

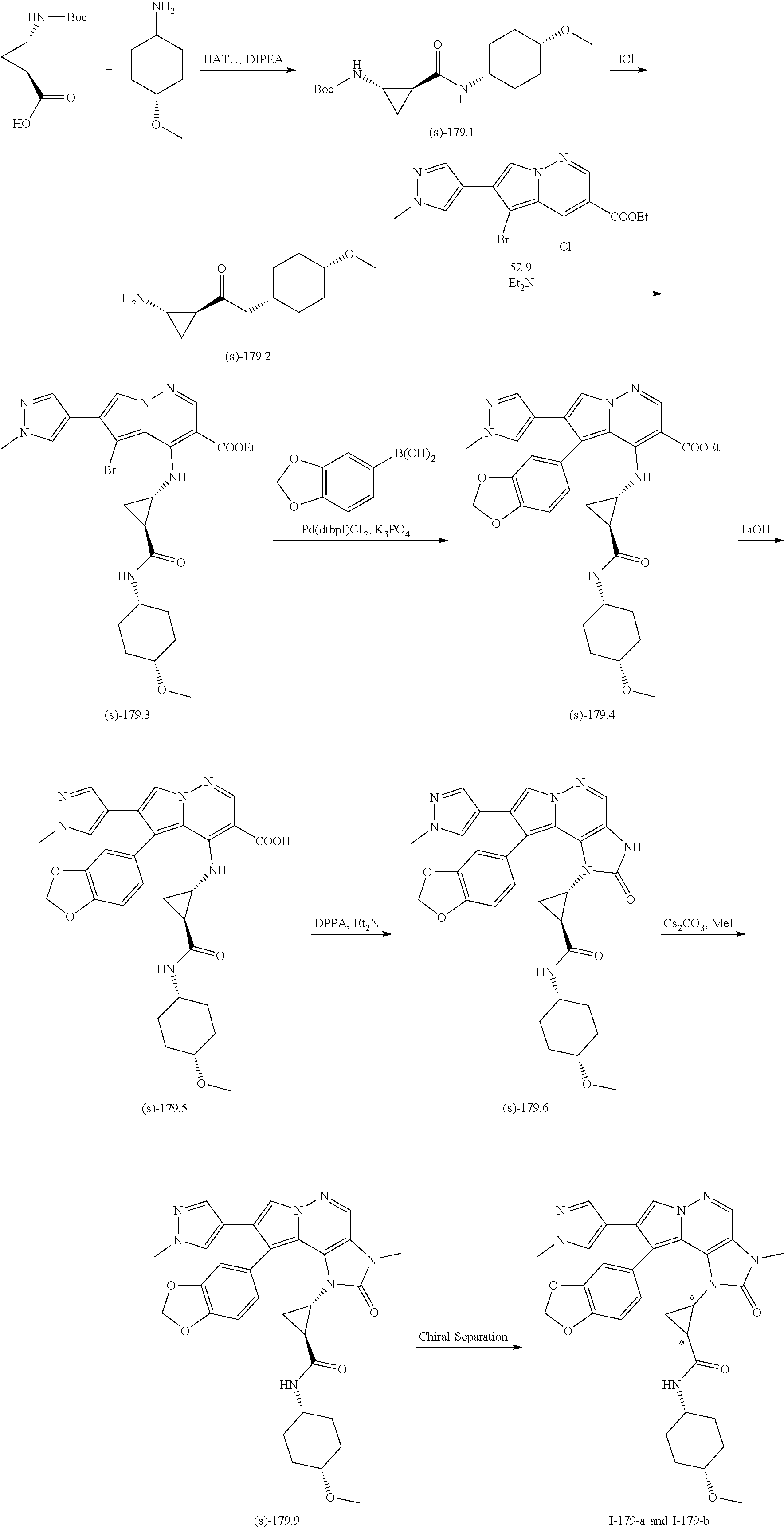

Synthesis of compound (+)-179.1. A solution of trans-2-((tert-butoxycarbonyl)amino)cyclopropane-1-carboxylic acid (2 g, 9.95 mmol, 1.0 equiv) and HATU (5.67 g, 14.92 mmol, 1.5 equiv) in DMF (10 mL) was stirred for 30 min at 0° C. To the solution were added N, N-diisopropylethylamine (3.85 g, 29.85 mmol, 3.0 equiv) and cis-4-methoxycyclohexan-1-amine (3.85 g, 29.85 mmol, 3.0 equiv) and stirred at rt for 1 h. It transferred into water and extracted with ethyl acetate. The organic layer was washed with brine, dried over sodium sulphate, and concentrated under reduced pressure. The residue was purified by flash column chromatography on silica gel (Combiflash®, 1.6% methanol in DCM) to afford (+)-179.1 (1.4 g, 45%).

Synthesis of compound (+)-179.2. To a solution of (+)-179.1 (1.4 g, 4.48 mmol, 1.0 equiv) in DCM (8 mL) was added hydrochloric acid in 1,4-dioxane (4 M, 14 mL) at 0° C. The reaction mixture was stirred at room temperature for 1 h. It was concentrated under reduced pressure to afford (±)-179.2 (0.82 g, 86%). It was used in the next step without further purification.

Synthesis of compound (±)-179.3. Compound (±)-179.3 was prepared from compound 52.9 and (±)-179.3 following the procedure described in the synthesis of compound 1.9. The product was purified by flash column chromatography on silica gel (30% ethyl acetate in hexane). MS(ES): m/z 559.16 and 561.16 [M+H]⁺.

Synthesis of compound (±)-179.4. Compound (±)-179.4 was prepared from compound (±)-179.3 and benzo[d][1,3]dioxol-5-ylboronic acid following the procedure described in the synthesis of compound 59.12. The product was purified by flash column chromatography on silica gel (55% ethyl acetate in hexane). MS(ES): m/z 601.5 [M+H]⁺.

Synthesis of compound (±)-179.5. Compound (±)-179.5 was prepared from compound (±)-179.4 following the procedure described in the synthesis of compound 1.10. The product was triturated with pentane, dried under reduced pressure and used in the next step without purification. MS(ES): m/z 573.62 [M+H]⁺.

Synthesis of compound (±)-179.6. Compound (±)-179.6 was prepared from compound (±)-179.5 following the procedure described in the synthesis of compound 1.11. The product was purified by flash column chromatography on silica gel (Combiflash®, 2.1% methanol in DCM). MS(ES): m/z 570.4 [M+H]⁺.

Synthesis of compound (±)-179. Compound (±)-179 was prepared from compound (±)-179.6 following the procedure described in the synthesis of compound 1.12. The product was purified by flash column chromatography on silica gel (1.8% methanol in DCM). MS(ES) 584.65 [M+H]⁺.

I-179-a and I-179-b. The racemate was separated by SFC (column: CHIRALPAK IB-N (250*30 mm), 5 μm; mobile phase: (A) Liquid CO₂. (B) 0.1% NH₃ in propane-2-ol: MeCN (50:50); flow rate: 100 mL/min) to afford first eluting fraction (I-179-a) and second eluting fraction (I-179-b). *The absolute configuration of the chiral center is not determined.

I-179-a: MS(ES): m/z 584.8 [M+H]⁺, ¹H-NMR (400 MHz, DMSO-d6): δ 8.48 (s, 1H), 8.16 (s, 1H), 7.41 (d, J=8.40 Hz, 1H), 7.21 (s, 1H), 7.09 (t, J=8.40 Hz, 1H), 6.93-6.84 (m, 2H), 6.16-6.12 (m, 2H), 5.10 (d, J=9.20 Hz, 1H), 3.77 (d, J=5.60 Hz, 3H), 3.35 (m, 3H), 3.11 (s, 1H), 2.76 (s, 1H), 2.71 (s, 3H), 2.08-1.99 (m, 2H), 1.73 (s, 2H), 1.54-1.51 (m, 1H), 1.43-1.37 (m, 2H), 1.14-1.12 (m, 1H), 1.11-1.09 (m, 2H), 0.92 (s, 2H).

I-179-b: MS(ES): m/z 584.2 [M+H]⁺, ¹H-NMR (400 MHz, DMSO-d6): δ 8.47 (s, 1H), 8.15 (s, 1H), 7.40 (d, J=8.40 Hz, 1H), 7.17 (s, 1H), 7.08 (t, J=9.20 Hz, 1H), 6.92-6.83 (m, 2H), 6.15-6.11 (m, 2H), 5.10 (d, J=8.00 Hz, 1H), 3.76 (d, J=5.20 Hz, 3H), 3.35 (m, 3H), 3.11 (s, 1H), 2.72 (s, 1H), 2.68 (s, 3H), 2.12-2.04 (m, 2H), 1.76-1.67 (m, 2H), 1.57-1.53 (m, 2H), 1.46-1.34 (m, 2H), 1.30-1.15 (m, 1H), 1.05-1.04 (m, 1H), 0.92 (s, 2H).

### Example I-180: 3-((1S,3S)-3-(9-(benzo[d][1,3]dioxol-5-yl)-8-(3-fluoro-1-methyl-1H-pyrazol-4-yl)-3-methyl-2-oxo-2,3-dihydro-1H-imidazo[4, 5-d]pyrrolo[1,2-b]pyridazin-1-yl)cyclopentyl)oxazolidin-2-one

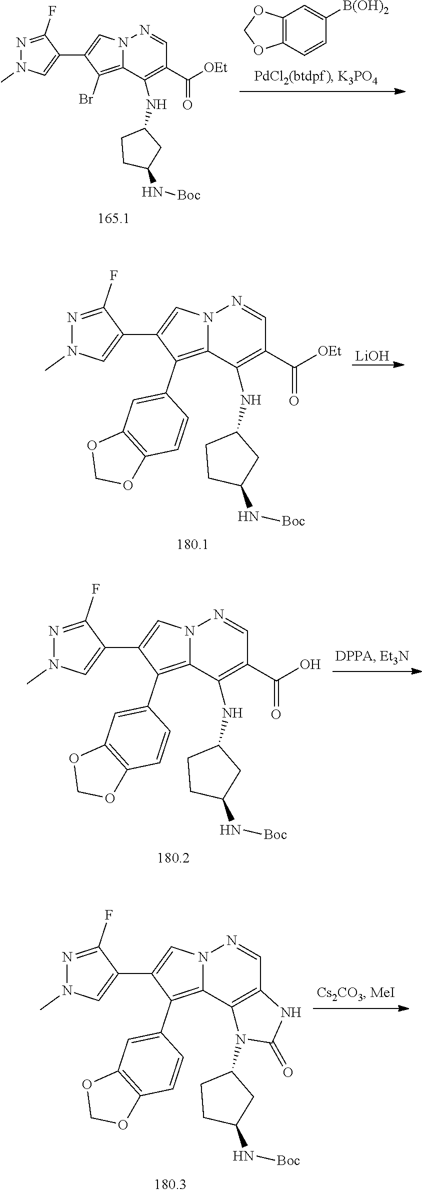

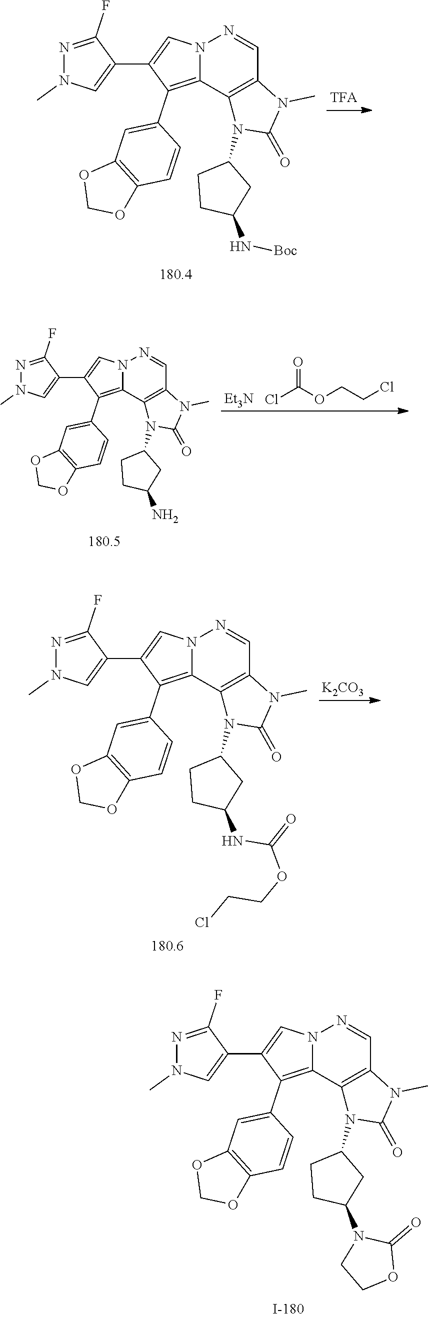

Synthesis of compound 180.1. Compound 180.1 was prepared from compound 165.1 and benzo[d][1,3]dioxol-5-ylboronic acid following the procedure described in the synthesis of compound 59.12. The product was purified by flash column chromatography on silica gel (54% ethyl acetate in hexane). MS(ES): m/z 607.4 [M+H]⁺.

Synthesis of compound 180.2. Compound 180.2 was prepared from compound 180.1 following the procedure described in the synthesis of compound 1.10. The product was used in the next step without purification. MS(ES): m/z 579.35 [M+H]⁺.

Synthesis of compound 180.3. Compound 180.3 was prepared from compound 180.2 following the procedure described in the synthesis of compound 1.11. The product was purified by flash column chromatography on silica gel (Combiflash®, 3.5% methanol in DCM. MS(ES): m/z 576.7 [M+H]⁺.

Synthesis of compound 180.4. Compound 180.4 was prepared from compound 180.3 following the procedure described in the synthesis of compound 1.12. The product was purified by flash column chromatography on silica gel (3.6% methanol in DCM). MS(ES): m/z 590.25 [M+H]⁺.

Synthesis of compound 180.5. Compound 180.5 was prepared from compound 180.4 following the procedure described in the synthesis of compound 1.14. The product was used in the next step without purification. MS(ES): m/z 490.20 [M+H]⁺.

Synthesis of compound 180.6. Compound 180.6 was prepared from compound 180.5 and 2-chloroethyl carbonochloridate following the procedure described in the synthesis of compound 9.7. The product was purified by flash column chromatography on silica gel (2.4% methanol in DCM). MS(ES): m/z 597.17 [M+H]⁺.

Synthesis of compound I-180. Compound I-180 was prepared from compound 180.6 following the procedure described in the synthesis of compound I-9. The product was purified by flash column chromatography on silica gel (Combiflash®, 3.2% methanol in DCM). MS (ES): m/z 560.27 [M+H]⁺; 1H-NMR (400 MHz, DMSO-d6): δ 8.45 (s, 1H), 7.95 (s, 1H), 7.10 (d, J=7.60 Hz, 1H), 6.98 (d, J=12.00 Hz, 1H), 6.91-6.83 (m, 2H), 6.17-6.15 (m, 2H), 4.35-4.26 (m, 4H), 3.78-3.77 (m, 1H), 3.63 (s, 3H), 3.38 (m, 4H), 2.13-2.06 (m, 2H), 1.78-1.76 (m, 1H), 1.56-1.54 (m, 1H), 1.49-1.36 (m, 1H), 1.10-1.01 (m, 1H).

### Example I-181: (1R,2R)—N-((1s,4S)-4-(9-(2,3-dihydrobenzo[b][1,4]dioxin-6-yl)-3-methyl-8-(1-methyl-1H-1,2,3-triazol-4-yl)-2-oxo-2,3-dihydro-1H-imidazo[4,5-d]pyrrolo[1,2-b]pyridazin-1-yl)-1-methylcyclohexyl)-2-fluorocyclopropane-1-carboxamide

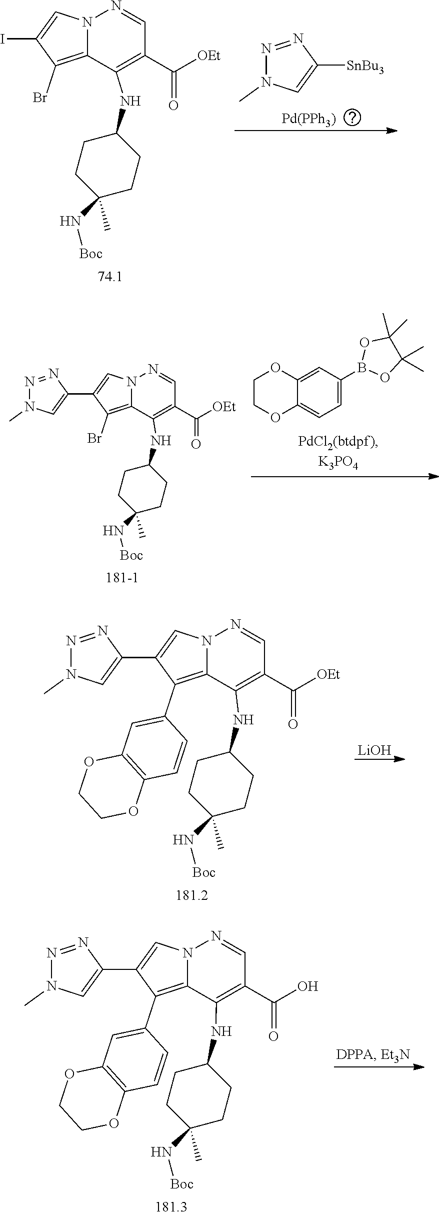

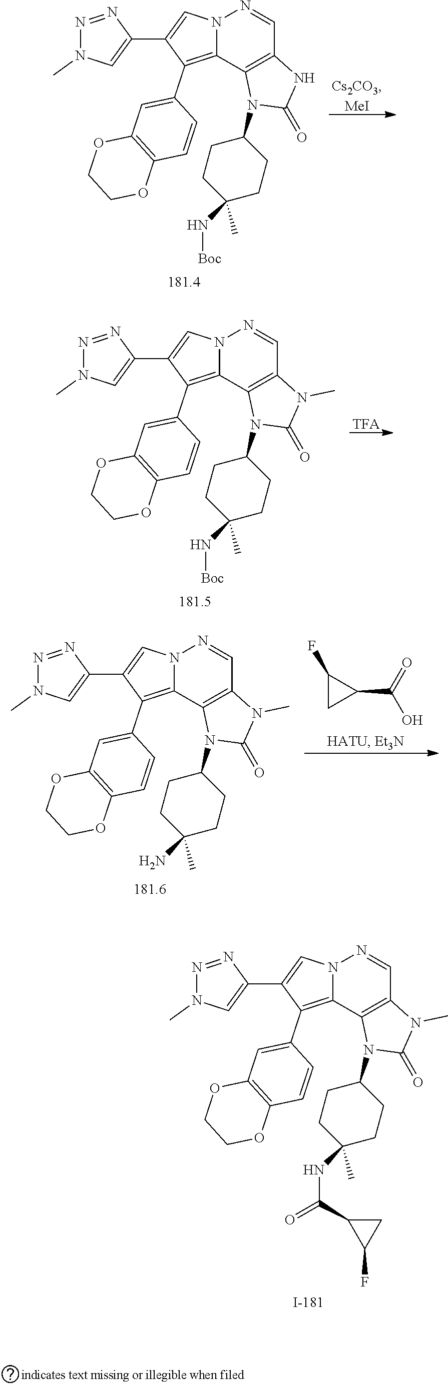

Synthesis of compound 181.1. Compound 181.1 was prepared from compound 74.1 and 1-methyl-4-(tributylstannyl)-1H-1,2,3-triazole following the procedure described in the synthesis of compound 172.1. The product was purified by flash column chromatography on silica gel (Combiflash®, 51% ethyl acetate in hexane). MS(ES): m/z 578.14 and 580.14 [M+H]⁺.

Synthesis of compound 181.2. Compound 181.2 was prepared from compound 181.1 and 2-(2,3-dihydrobenzo[b][1,4]dioxin-6-yl)-4,4,5,5-tetramethyl-1,3,2-dioxaborolane following the procedure described in the synthesis of compound 59.12. The product was purified by flash column chromatography on silica gel (Combiflash®, 50% ethyl acetate in n-hexane). MS(ES): m/z 632.9 [M+H]⁺.

Synthesis of compound 181.3. Compound 181.3 was prepared from compound 181.2 following the procedure described in the synthesis of compound 1.10. The product was used in the next step without purification. MS(ES): m/z 604.5 [M+H]⁺.

Synthesis of compound 181.4. Compound 181.4 was prepared from compound 181.3 following the procedure described in the synthesis of compound 1.11. The product was purified by flash column chromatography on silica gel (Combiflash®, 2.4% methanol in DCM). MS(ES): m/z 601.7 [M+H]⁺.

Synthesis of compound 181.5. Compound 181.5 was prepared from compound 181.4 following the procedure described in the synthesis of compound 1.12. The product was purified by flash column chromatography on silica gel (1.6% methanol in DCM). MS(ES): m/z 615.7 [M+H]⁺.

1. A compound of formula I: 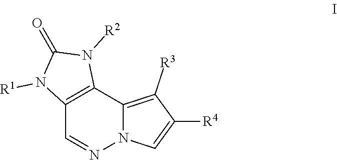
or a pharmaceutically acceptable salt thereof, wherein:
R¹ is hydrogen or optionally substituted C₁-C₆ aliphatic;
R² is C₃-C₁₂ cycloaliphatic, C₁-C₆ aliphatic, 2- to 10-membered heteroaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R² is optionally substituted with one or more instances of -L-R⁵ or R⁷;
each L is independently selected from the group consisting of a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, and —OC(S)—;
each R⁵ is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, and optionally substituted C₆-C₁₀ aryl;
each R⁷ is independently selected from the group consisting of halogen, —OH, —SH, and —NH₂;
R³ is 5- to 12-membered heteroaryl comprising 1 to 4 heteroatoms selected from N, O, and S, 4- to 12-membered heterocycle comprising 1 to 4 heteroatoms selected from N, O, and S, C₆-C₁₂ aryl, C₃-C₁₂ cycloaliphatic, or C₁-C₆ aliphatic, wherein R³ is optionally substituted with one or more instances of R^{3a}; or
R² and R³ are connected by *-L^{a}-R⁶-L^{b}-**, wherein * represents a point of attachment to R², and ** represents a point of attachment to R³;
L^{a} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
L^{b} is a bond, —N(R^{a})C(O)N(R^{a})—, —N(R^{a})C(O)O—, —OC(O)N(R^{a})—, —C(O)N(R^{a})—, —N(R^{a})C(O)—, —N(R^{a})—, —O—, —C(O)—, —C(O)O—, —OC(O)—, —N(R^{a})C(S)O—, —OC(S)N(R^{a})—, —C(S)N(R^{a})—, —N(R^{a})C(S)—, —C(S)—, —C(S)O—, or —OC(S)—;
R⁶ is optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, or optionally substituted C₆-C₁₀ aryl;
R⁴ is C₁-C₆ aliphatic, —CN, 2- to 10-membered heteroaliphatic, C₃-C₁₂ cycloaliphatic, 4- to 10-membered heterocycle comprising 1 to 4 heteroatoms selected from N, O, and S, 5- to 10-membered heteroaryl comprising 1 to 4 heteroatoms selected from N, O, and S, or C₆-C₁₀ aryl, wherein R⁴ is optionally substituted with one or more instances of R^{4a};
each R^{3a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a};
each R^{4a} is independently selected from the group consisting of optionally substituted C₁-C₆ aliphatic, optionally substituted 2- to 10-membered heteroaliphatic, optionally substituted C₃-C₁₂ cycloaliphatic, optionally substituted 4- to 10-membered heterocycle comprising 1 to 4 heteroatoms selected from N, O, and S, optionally substituted 5- to 10-membered heteroaryl comprising 1 to 4 heteroatoms selected from N, O, and S, optionally substituted C₆-C₁₀ aryl, halogen, oxo, —CN, —NO₂, —C(O)R^{a}, —C(O)N(R^{a})₂, —OC(O)R^{a}, —OC(O)N(R^{a})₂, —N(R^{a})C(O)R^{a}, —N(R^{a})C(O)OR^{a}, —N(R^{a})C(O)N(R^{a})₂, —S(O)₂N(R^{a})₂, —N(R^{a})S(O)₂R^{a}, —S(O)₂R^{a}, and —OR^{a}; and
each R^{a} is independently selected from hydrogen, optionally substituted C₁-C₆ aliphatic, optionally substituted 2- to 10-membered heteroaliphatic, optionally substituted 5- to 12-membered heteroaryl comprising 1 to 4 heteroatoms selected from N, O, and S, and optionally substituted 4- to 12-membered heterocycle comprising 1 to 4 heteroatoms selected from N, O, and S.

2. (canceled)

3. The compound of claim 1, wherein R¹ is optionally substituted C₁-C₆ aliphatic.

4-5. (canceled)

6. The compound of claim 1, wherein R² is C₃-C₇monocyclic cycloaliphatic or 4- to 6-membered monocyclic heterocycle comprising 1 to 2 heteroatoms selected from N, O, and S; and wherein R² is substituted with 1-2 instances of -L-R⁵ or R⁷.

7-9. (canceled)

10. The compound of claim 1, wherein R² is:

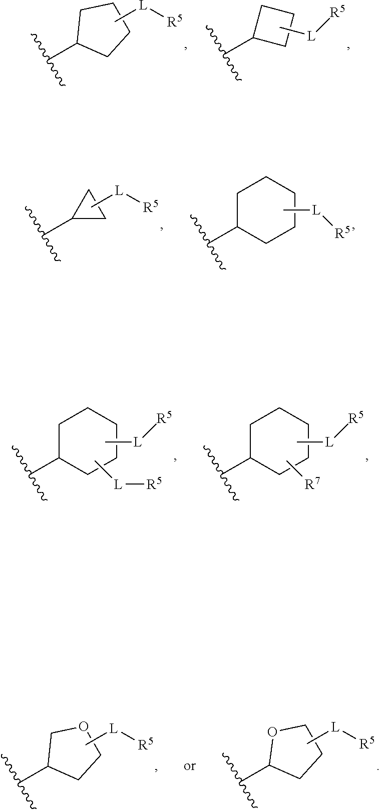

11. (canceled)

12. The compound of claim 1, wherein each L is independently selected from a bond, —N(R^{a})C(O)O—, —OC(O)N(R^{a})—, —C(O)N(R^{a})—, —N(R^{a})C(O)—, —O—, and —C(O)—.

13. (canceled)

14. The compound of claim 1, wherein each R⁵ is independently selected from optionally substituted C₁-C₆ aliphatic, optionally substituted C₃-C₆ cycloaliphatic, optionally substituted 4- to 6-membered heterocycle comprising 1 to 2 heteroatoms selected from N, O, and S, and optionally substituted 5- to 6-membered heteroaryl comprising 1 to 4 heteroatoms selected from N, O, and S.

15. The compound of claim 1, wherein each -L-R⁵ is independently selected from: —CH₃, —CH₂OH, —CH₂OCH₃,

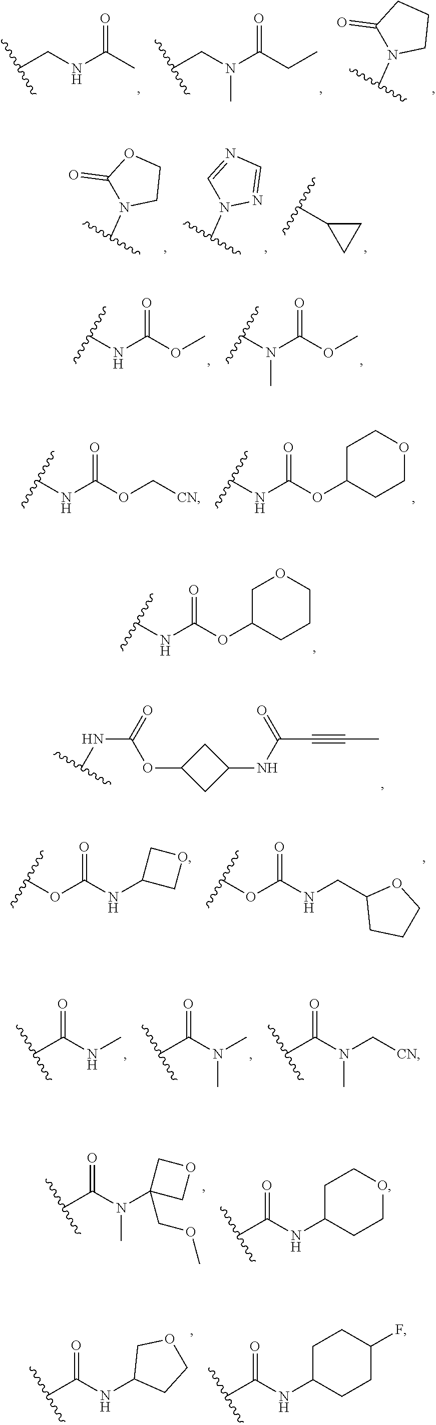

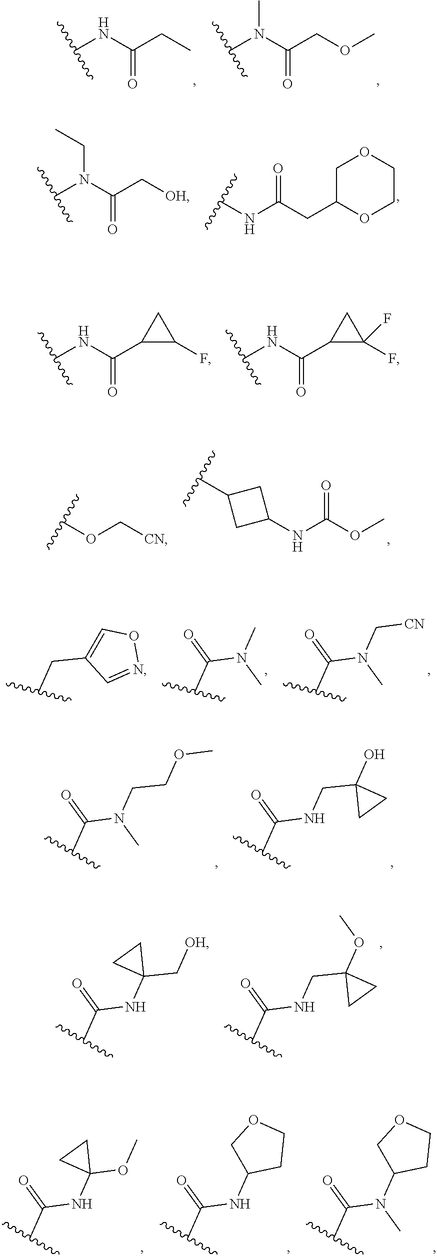

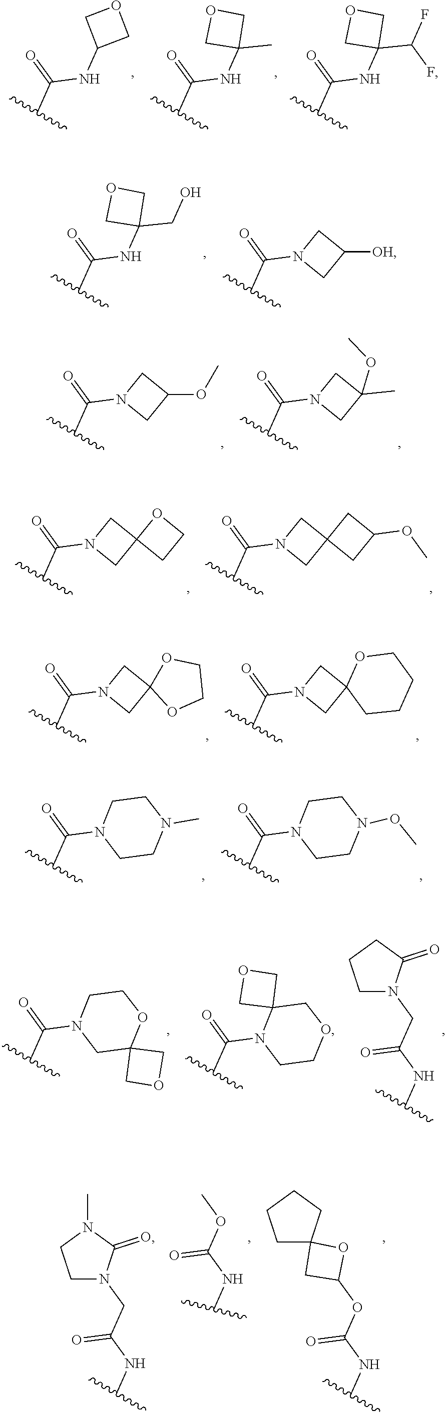

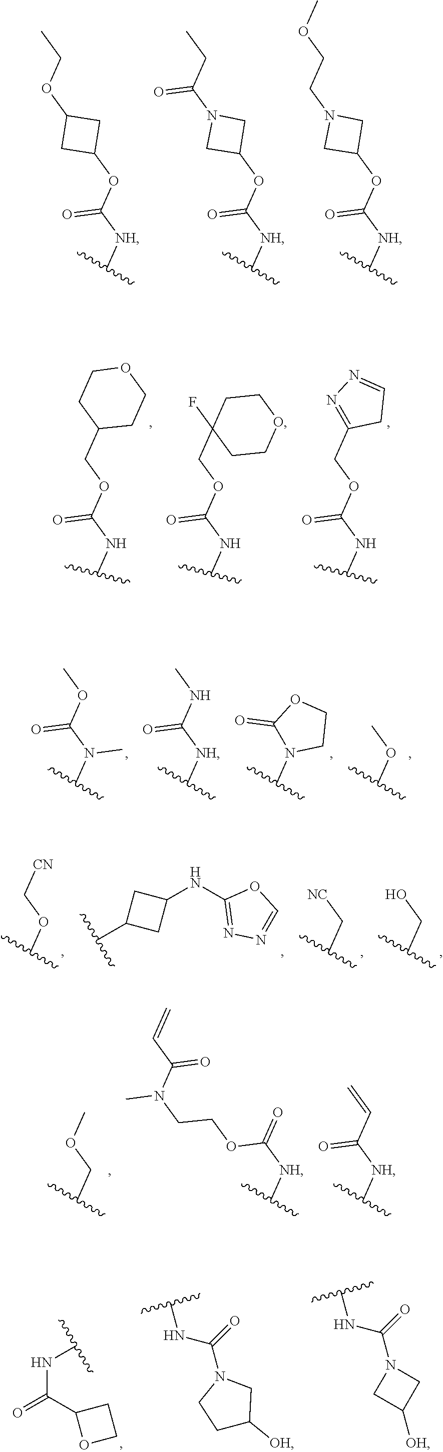

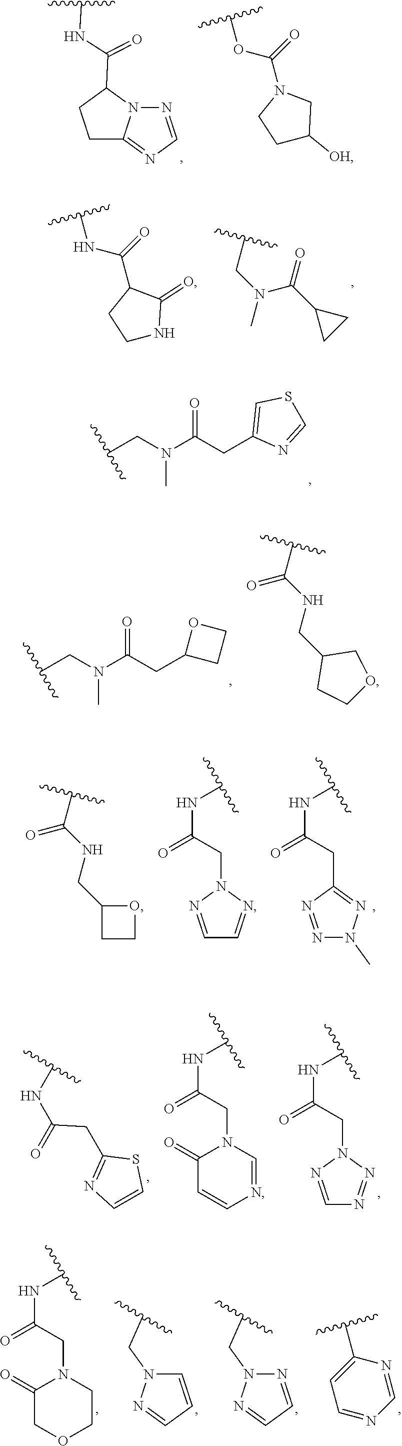

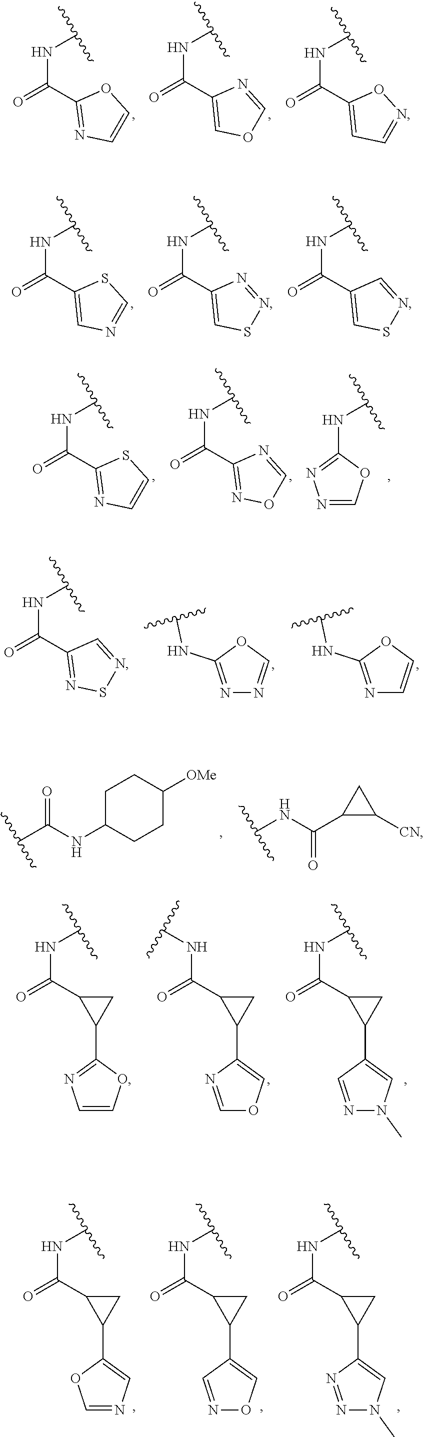

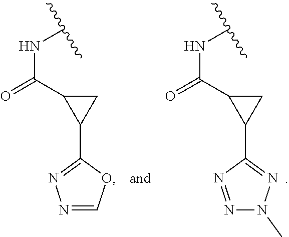 [SMILES: *NC(=O)C1CC1c1nnco1 ; *NC(=O)C1CC1c1nnn(C)n1]

16. (canceled)

17. The compound of claim 1, wherein R² optionally substituted with one or more -L-R⁵ or R⁷ is selected from:

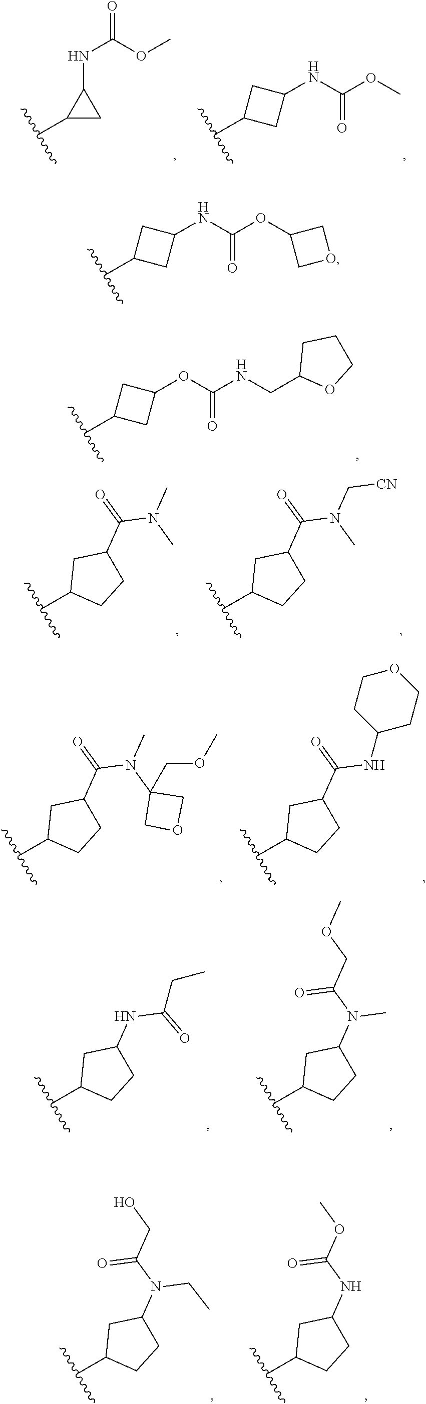

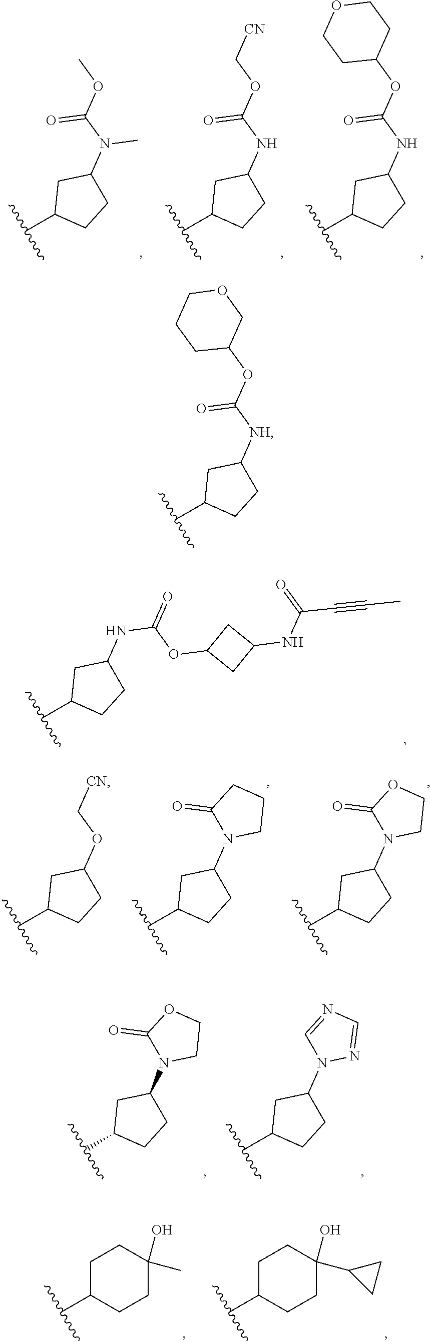

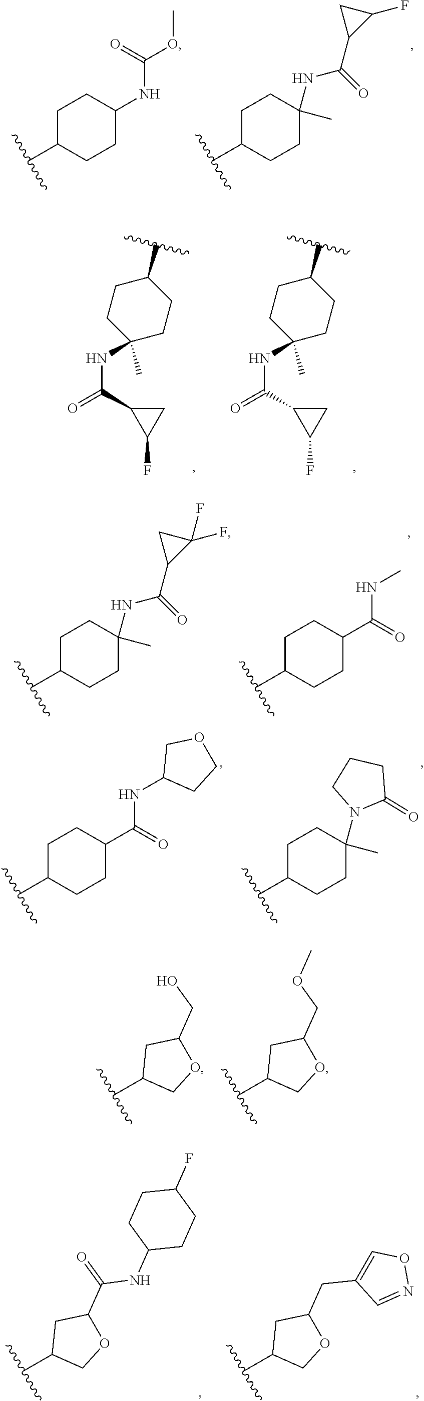

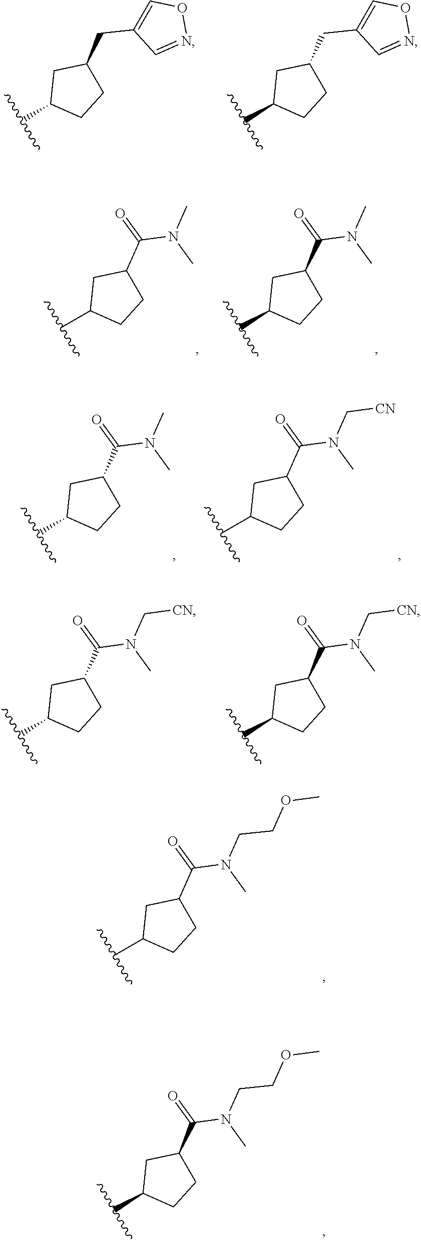

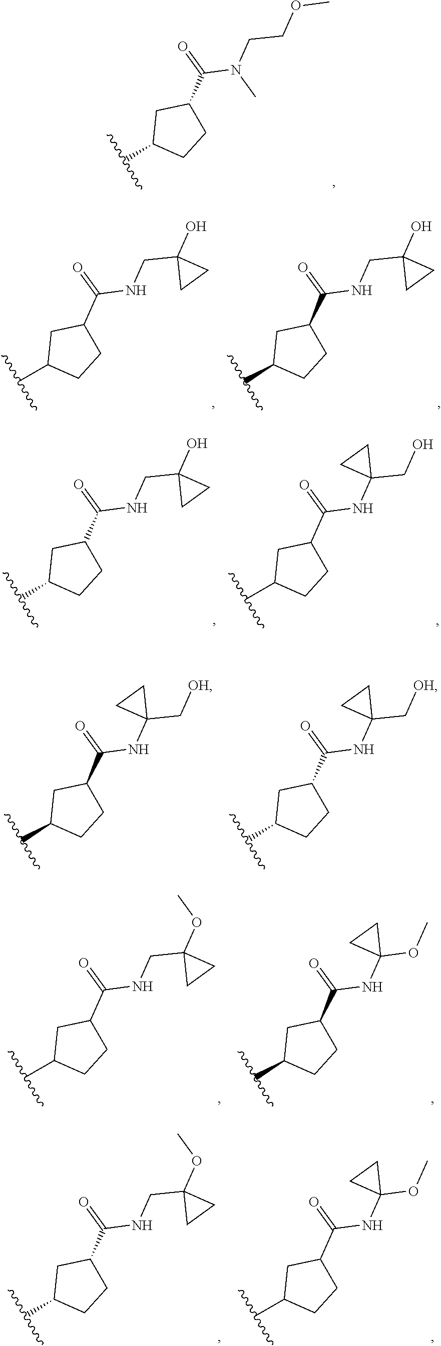

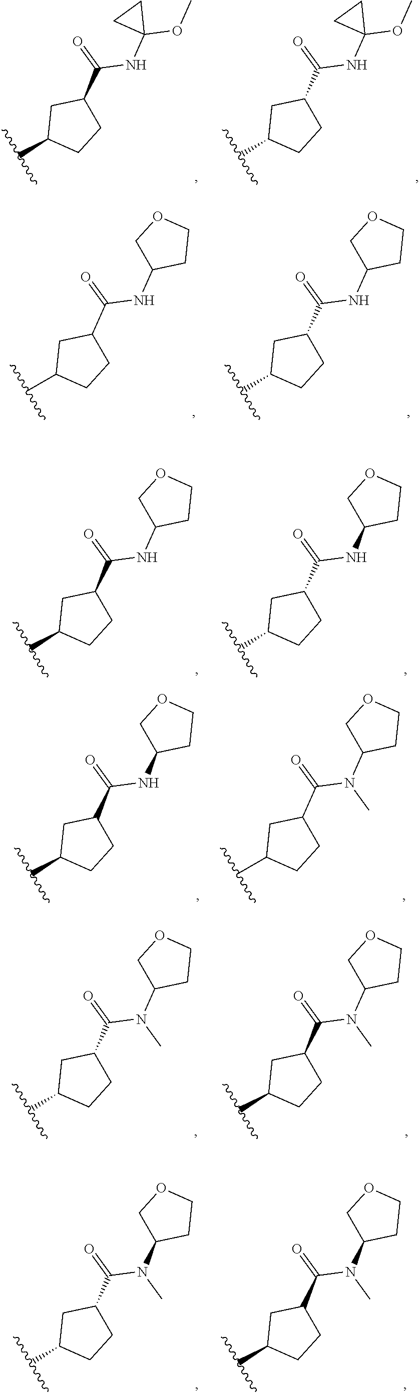

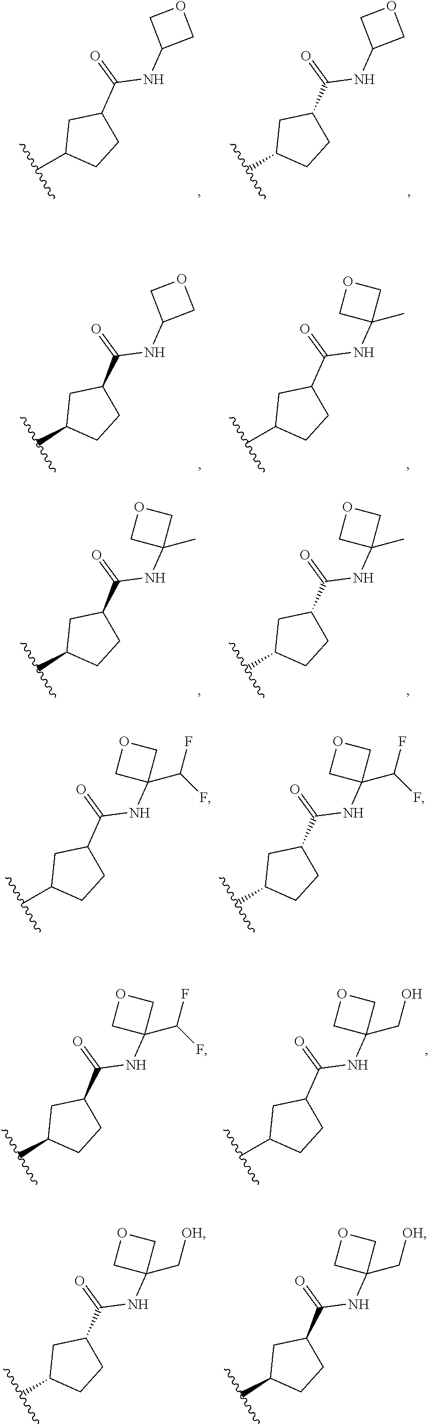

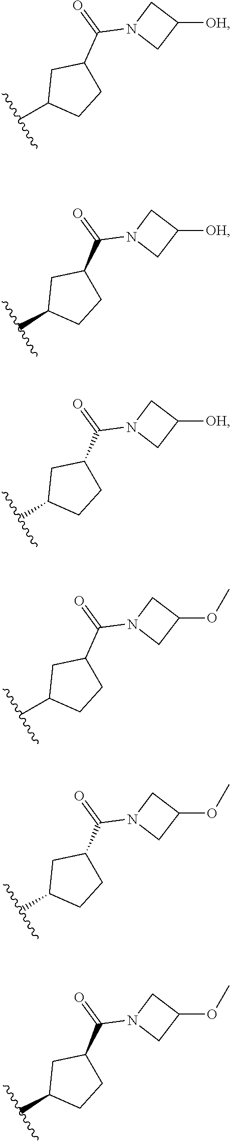 [SMILES: *C1CCC(C(=O)N2CC(O)C2)C1 ; *[C@@H]1CC[C@H](C(=O)N2CC(O)C2)C1 ; *[C@H]1CC[C@@H](C(=O)N2CC(O)C2)C1 ; *C1CCC(C(=O)N2CC(OC)C2)C1 ; *[C@H]1CC[C@@H](C(=O)N2CC(OC)C2)C1 ; *[C@@H]1CC[C@H](C(=O)N2CC(OC)C2)C1]

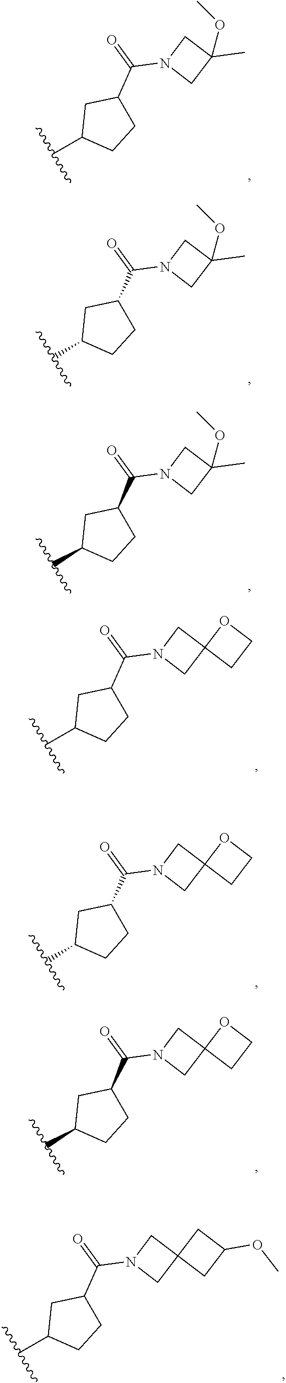

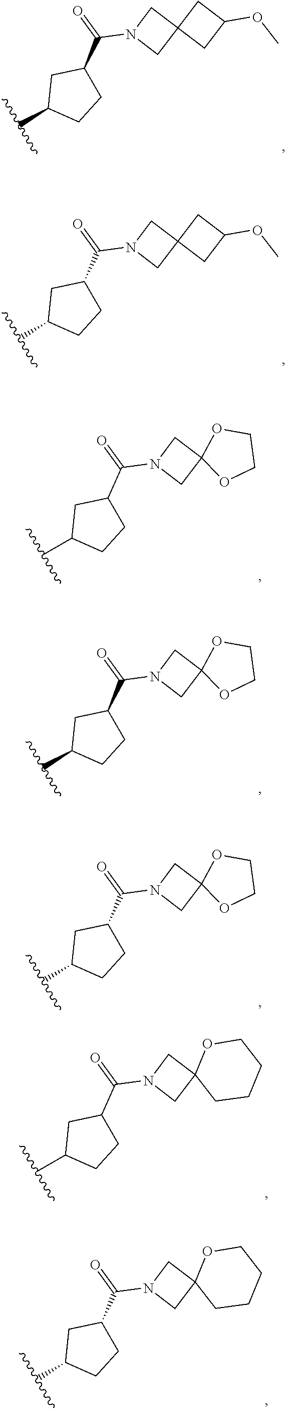

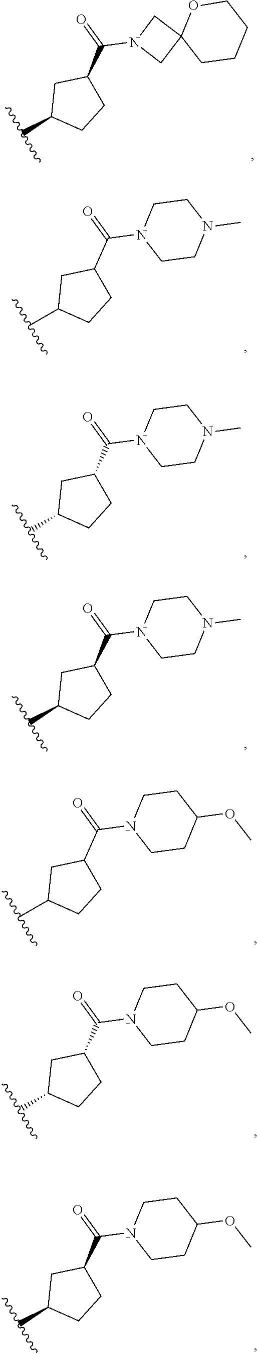

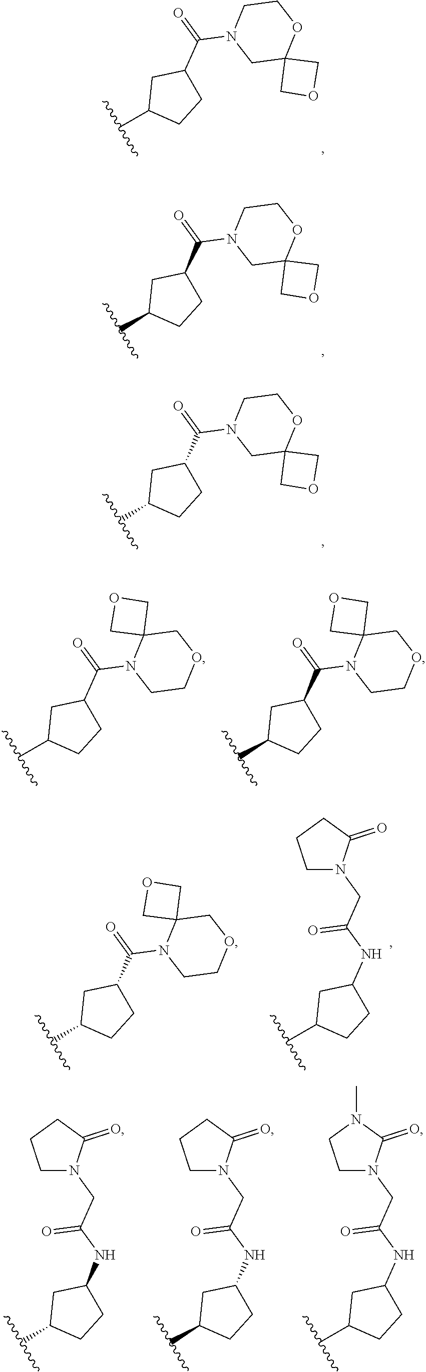

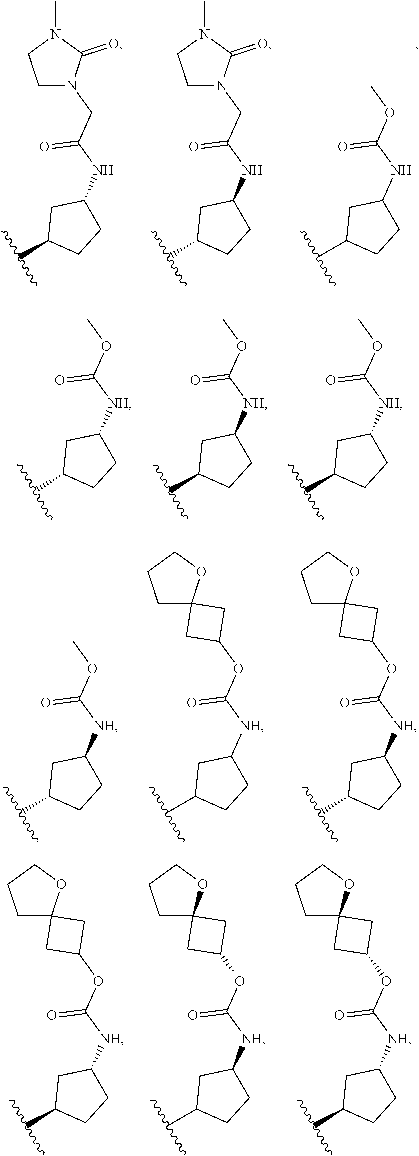

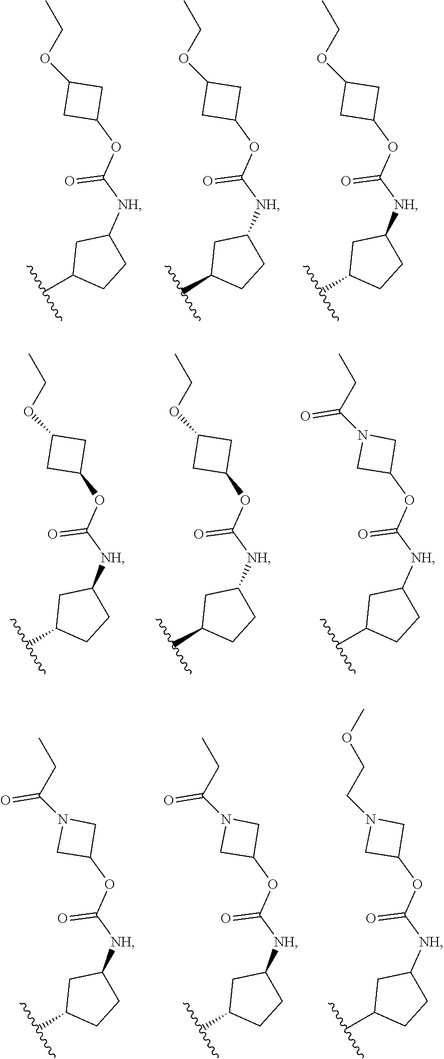

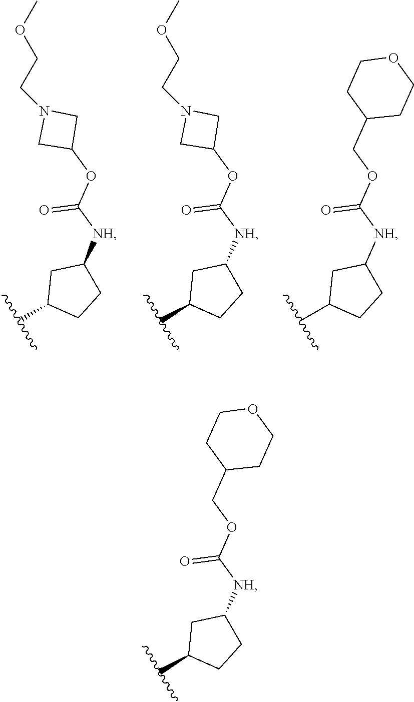 [SMILES: *[C@H]1CC[C@H](NC(=O)OC2CN(CCOC)C2)C1 ; *[C@@H]1CC[C@@H](NC(=O)OC2CN(CCOC)C2)C1 ; *C1CCC(NC(=O)OCC2CCOCC2)C1 ; *[C@@H]1CC[C@@H](NC(=O)OCC2CCOCC2)C1]

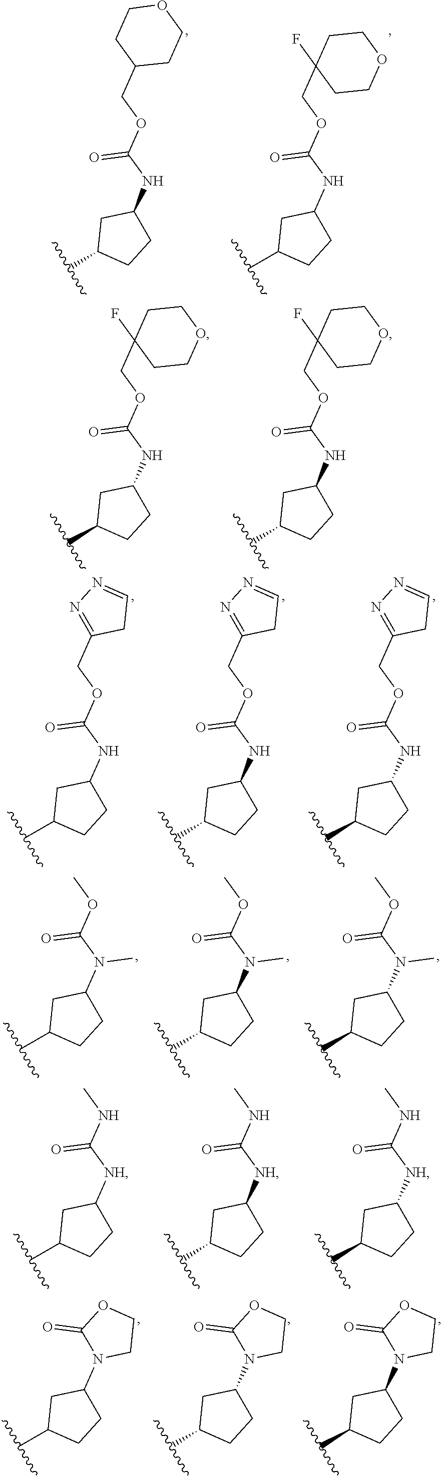

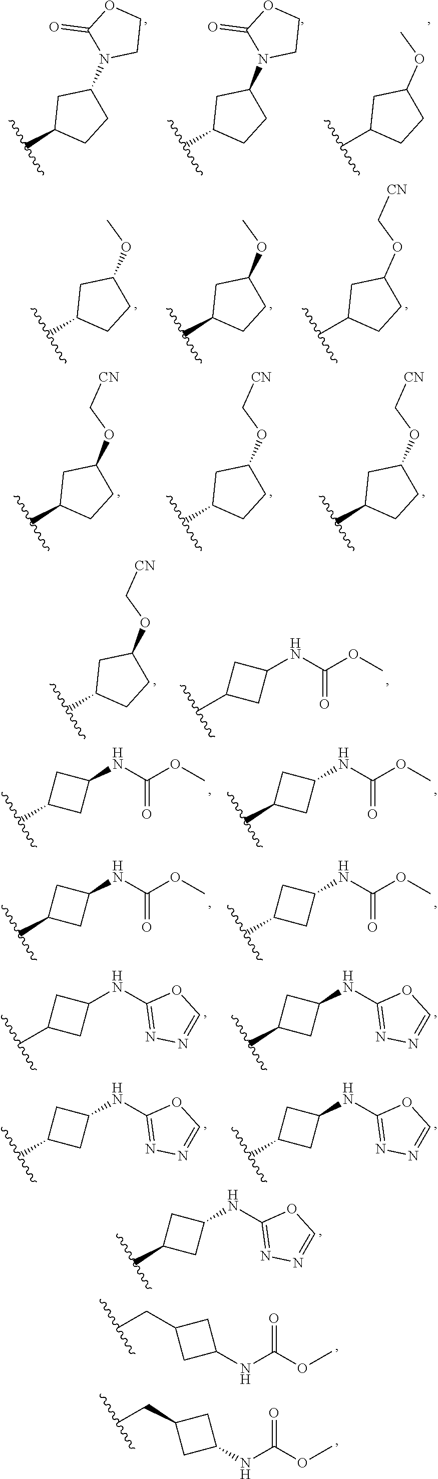

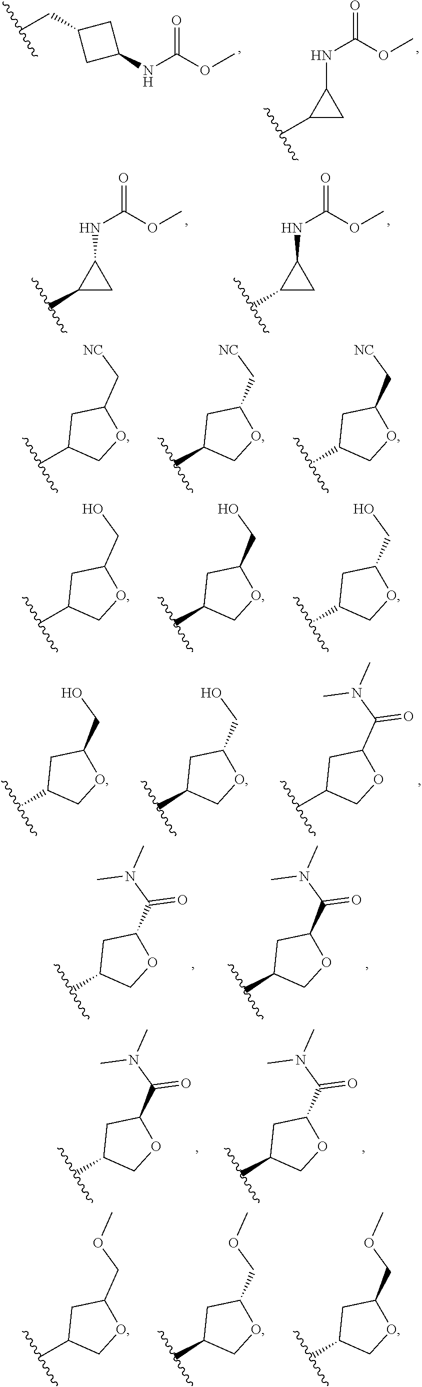

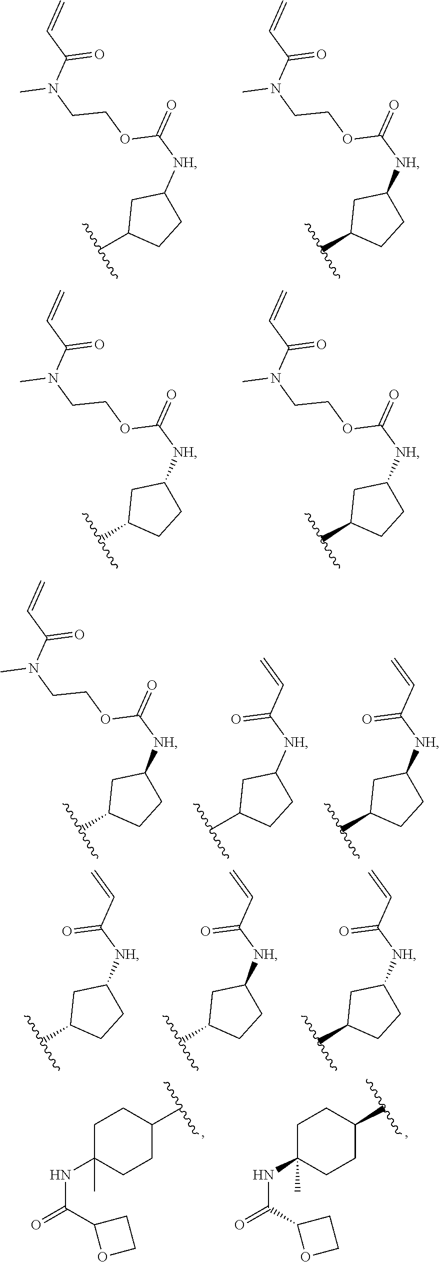

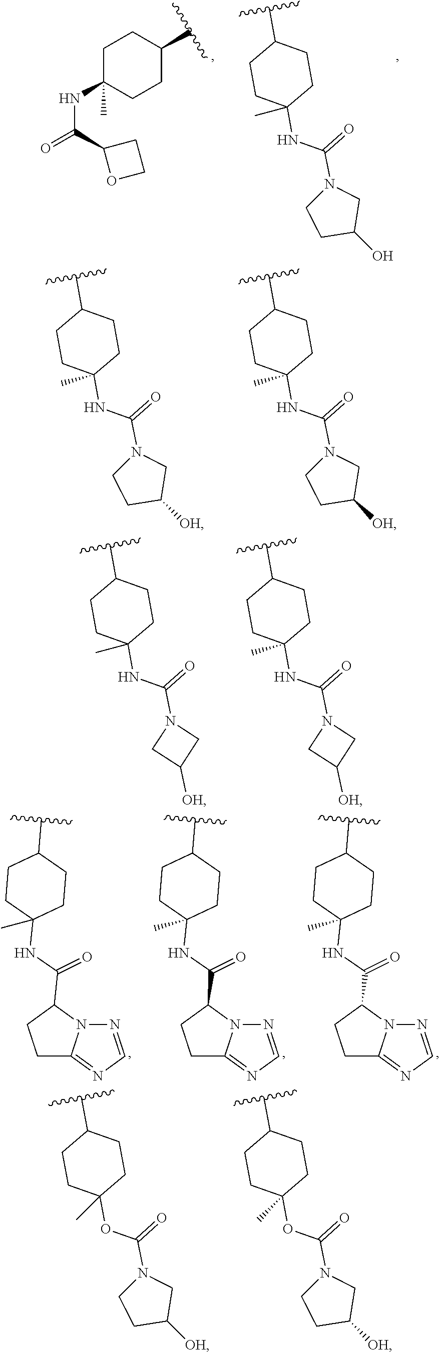

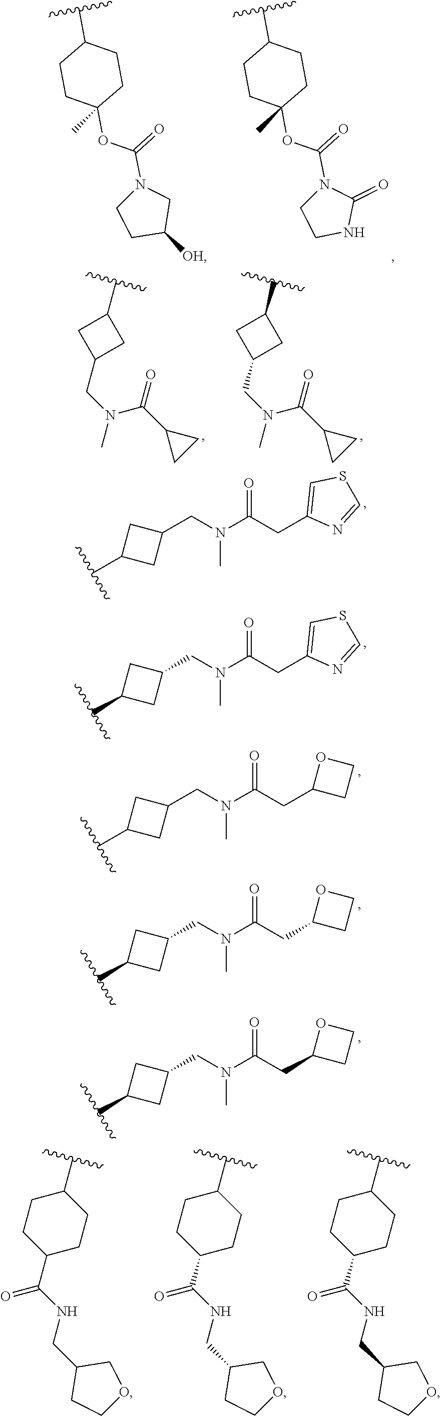

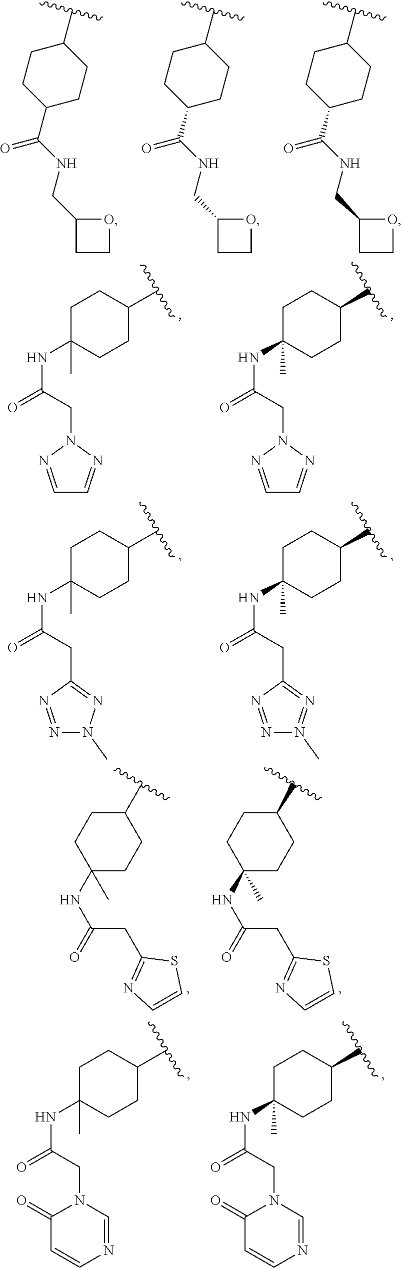

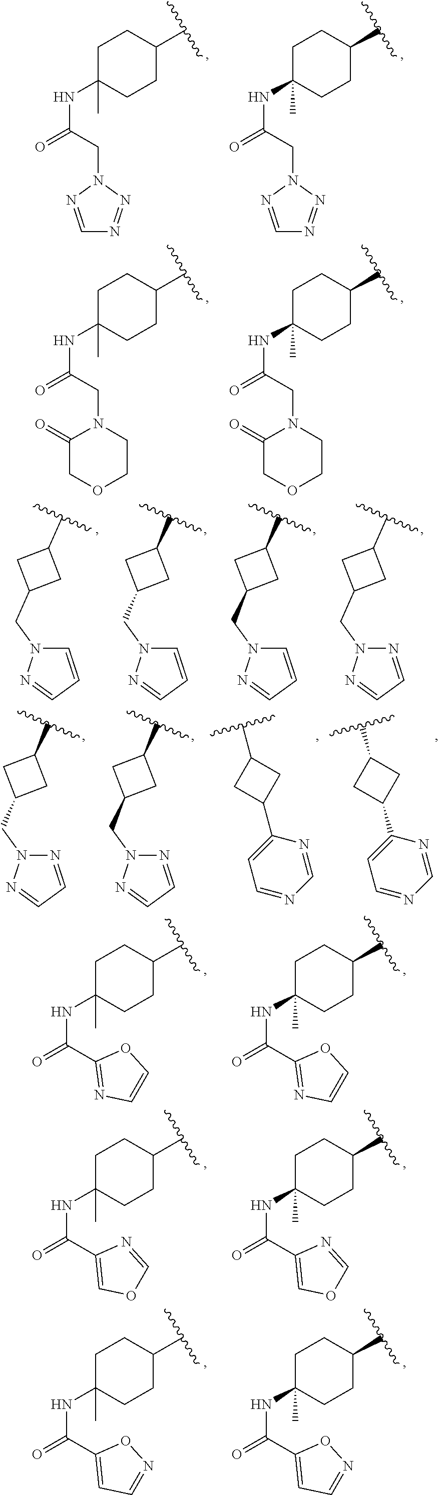

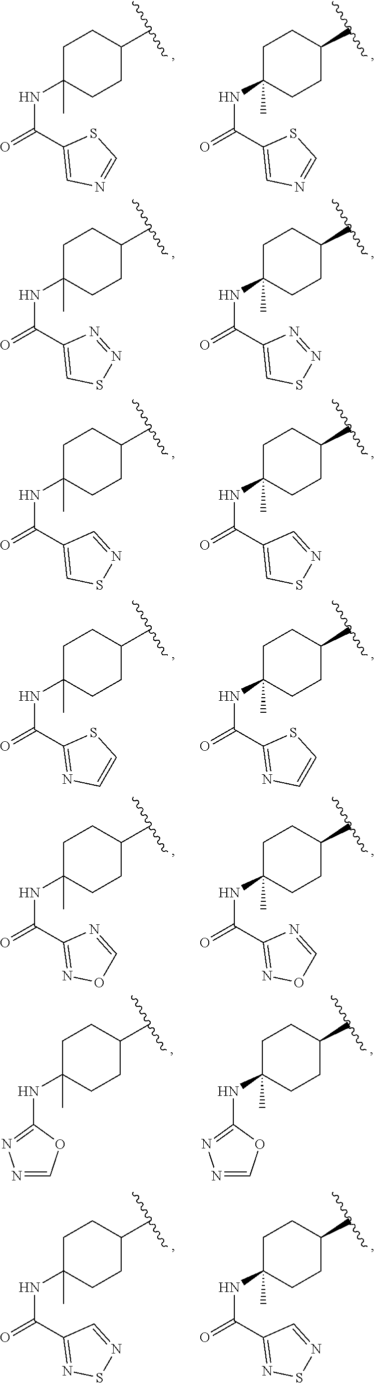

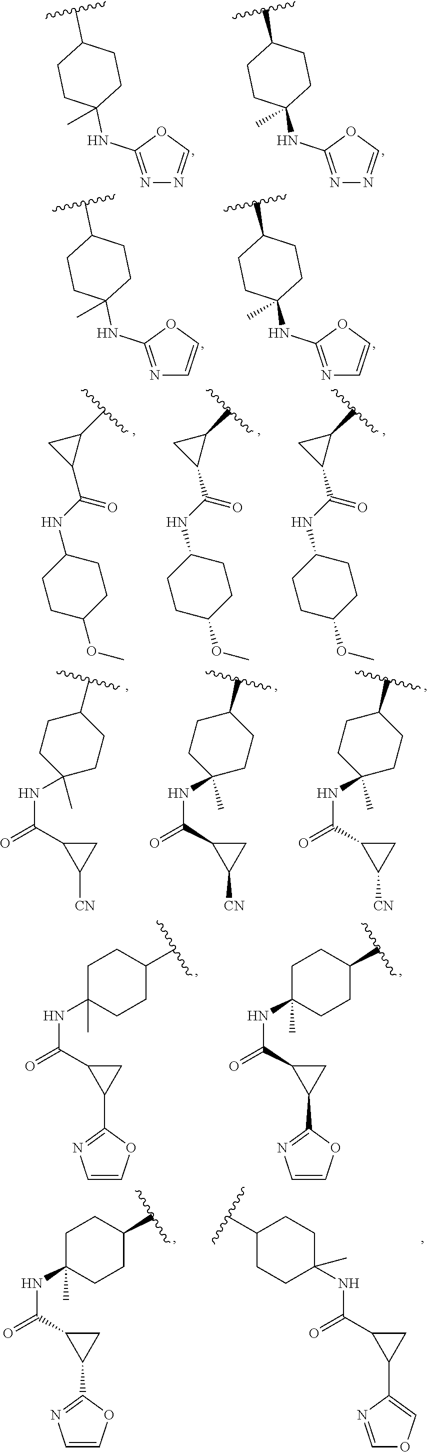

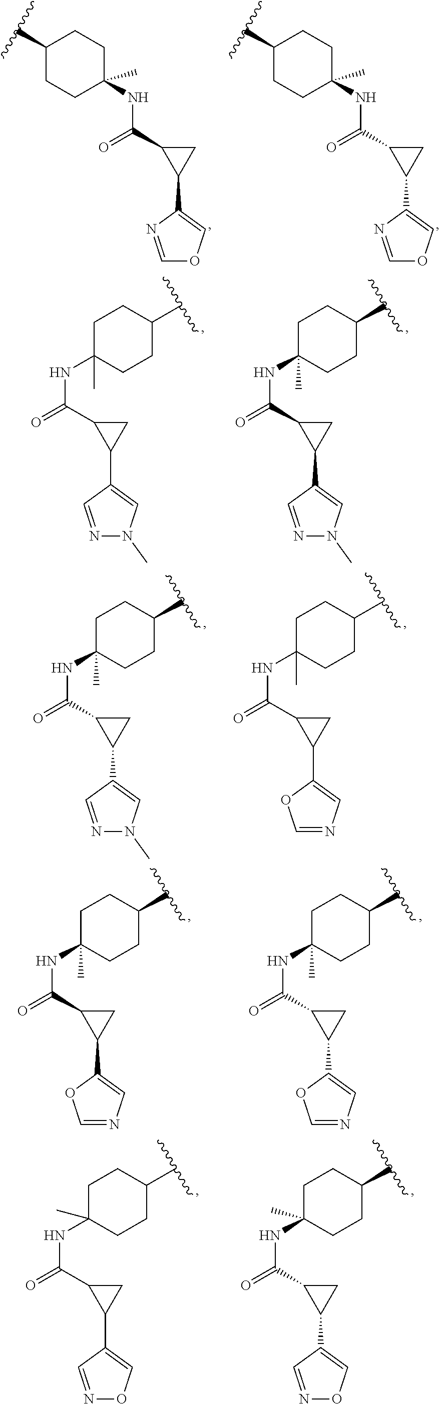

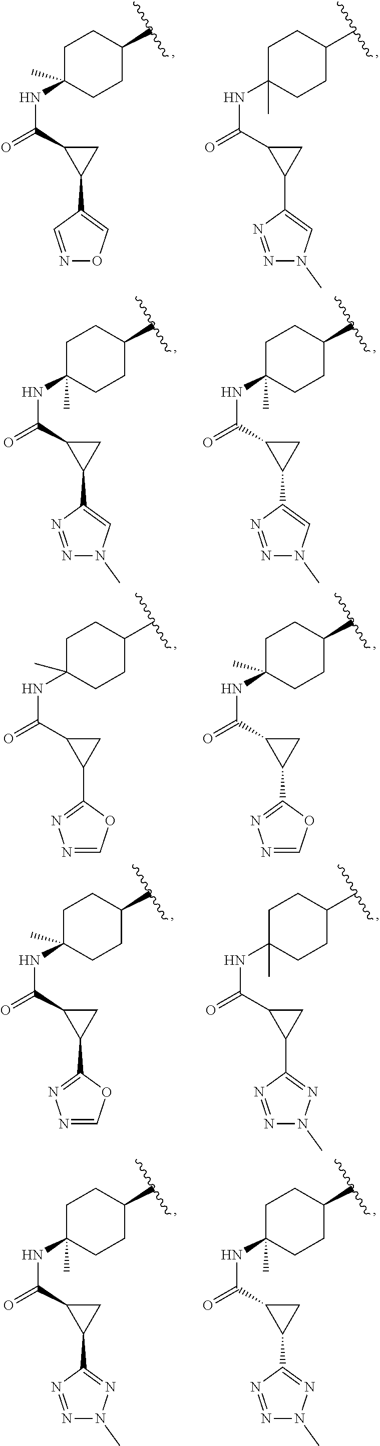

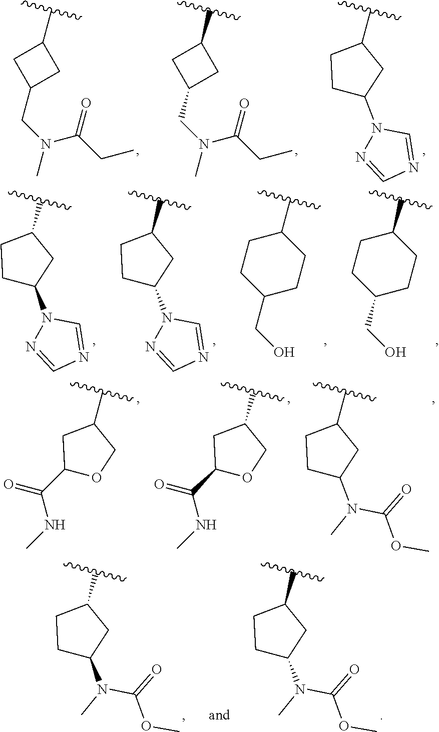

18. The compound of claim 1, wherein R³ is 5- to 10-membered heteroaryl comprising 1 to 4 heteroatoms selected from N, O, and S, 9- to 10-membered heterocycle comprising 1 to 4 heteroatoms selected from N, O, and S, or phenyl, wherein R³ is optionally substituted with 0-3 instances of R^{3a}.

19-24. (canceled)

25. The compound of claim 1, wherein each R^{3a} is independently selected from optionally substituted C₁-C₆ aliphatic, optionally substituted C₃-C₆ cycloaliphatic, halogen, —C(O)N(R^{a})₂, and —OR^{a}.

26. The compound of claim 1, wherein R³ optionally substituted with one or more instances of R^{3a} is:

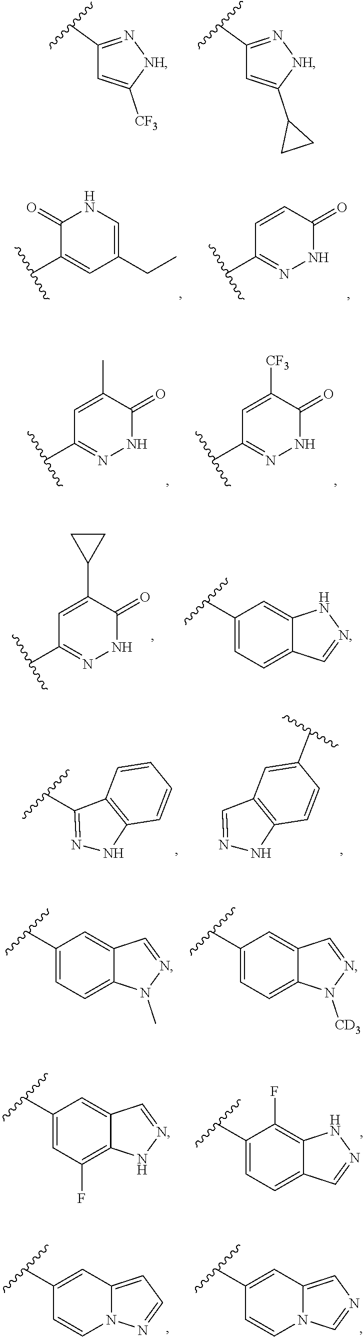

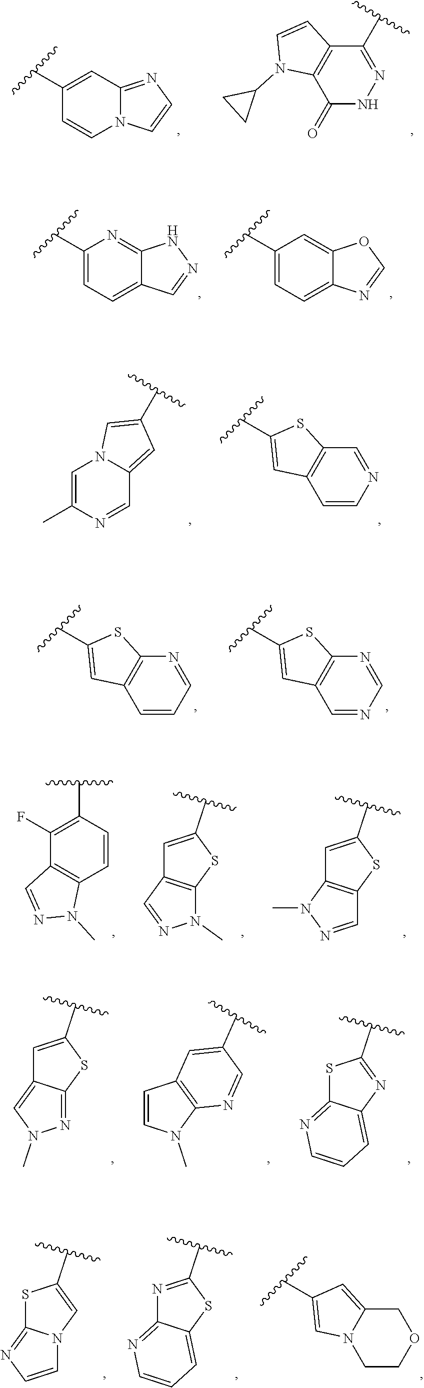

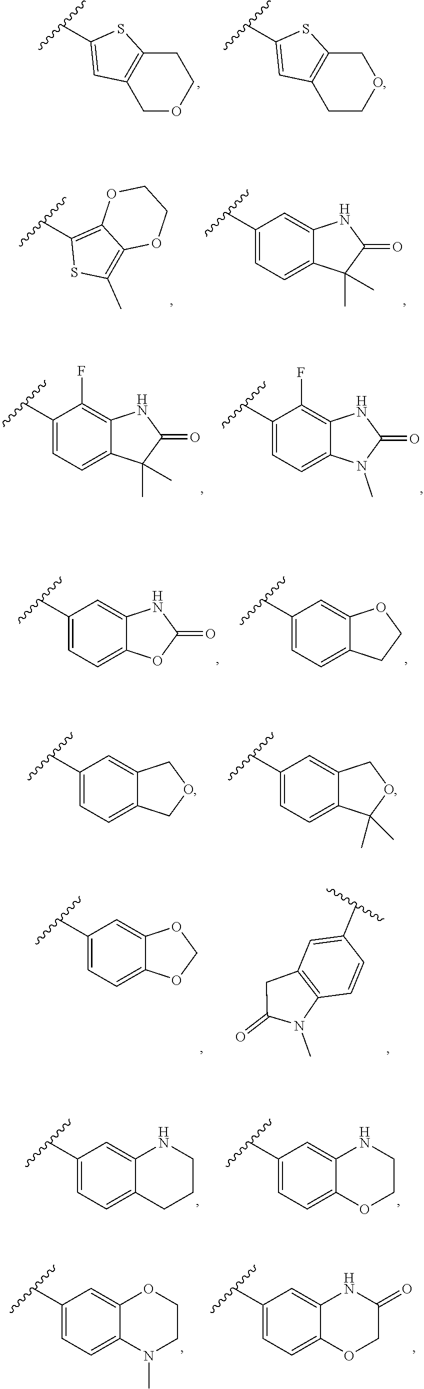

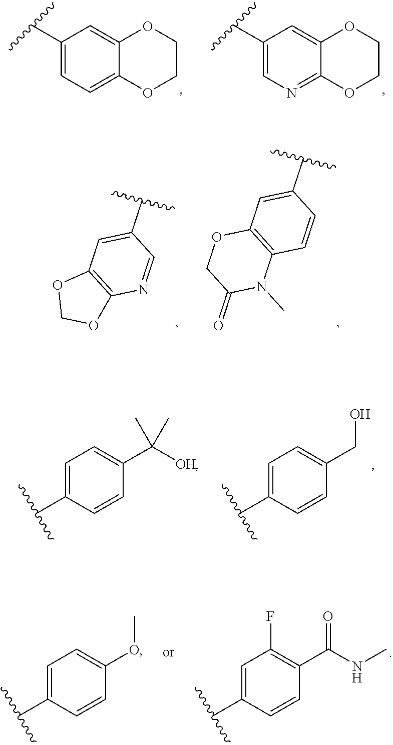

27-32. (canceled)

33. The compound of claim 1, wherein R⁴ is 5- to 10-membered heteroaryl comprising 1 to 4 heteroatoms selected from N, O, and S or 4- to 10-membered heterocycle comprising 1 to 4 heteroatoms selected from N, O, and S, and wherein R⁴ is optionally substituted with 0-3 instances of R^{4a}.

34-36. (canceled)

37. The compound of claim 1, wherein each R^{4a} is independently selected from optionally substituted C₁-C₆ aliphatic, —OR^{a}, and halogen.

38. The compound of claim 1, wherein R⁴ optionally substituted with one or more instances R^{4a} is selected from:

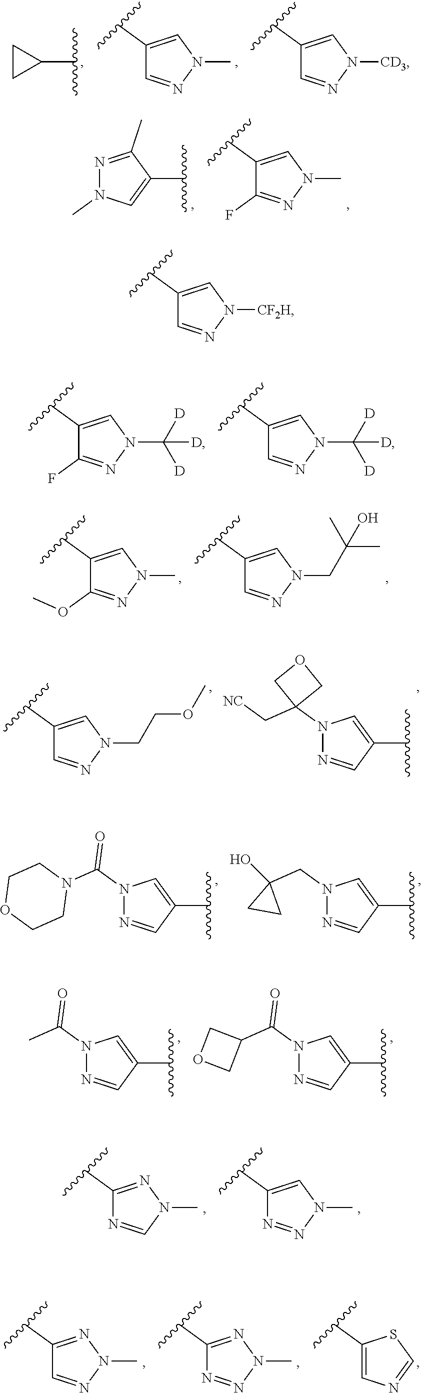

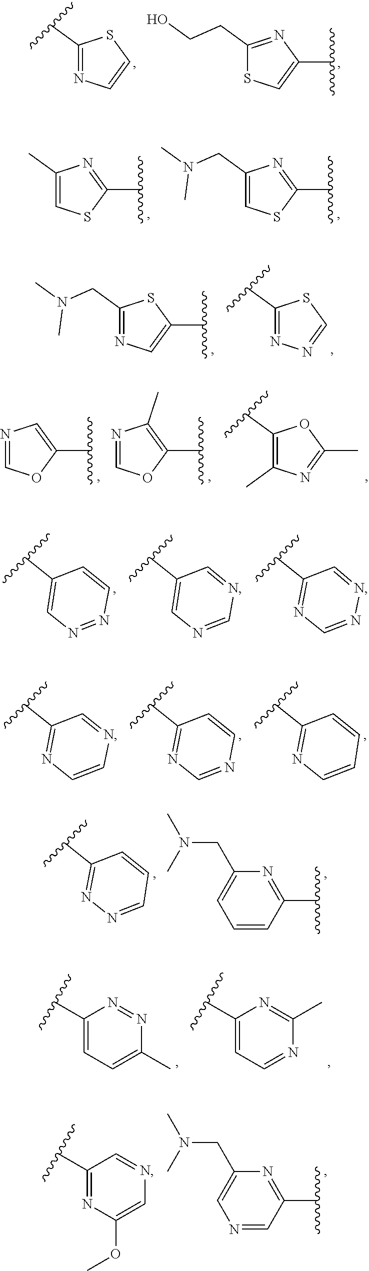

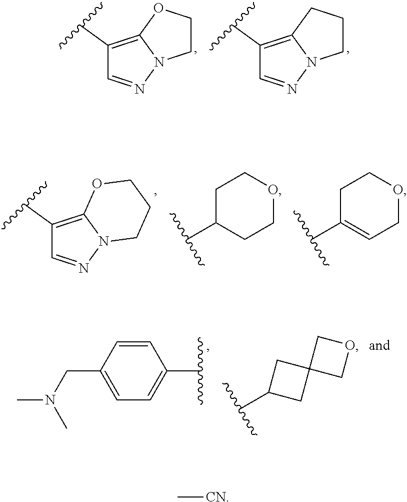

39-40. (canceled)

41. The compound of claim 1, wherein the compound is represented by formula IV-2 or IV-3: 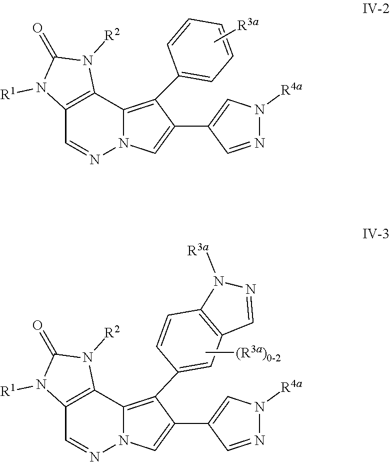
or a pharmaceutically acceptable salt thereof.

42-43. (canceled)

44. The compound of claim 1, wherein the compound is represented by formula VII-1, VII-2, VII-3, VII-4, or VII-5: 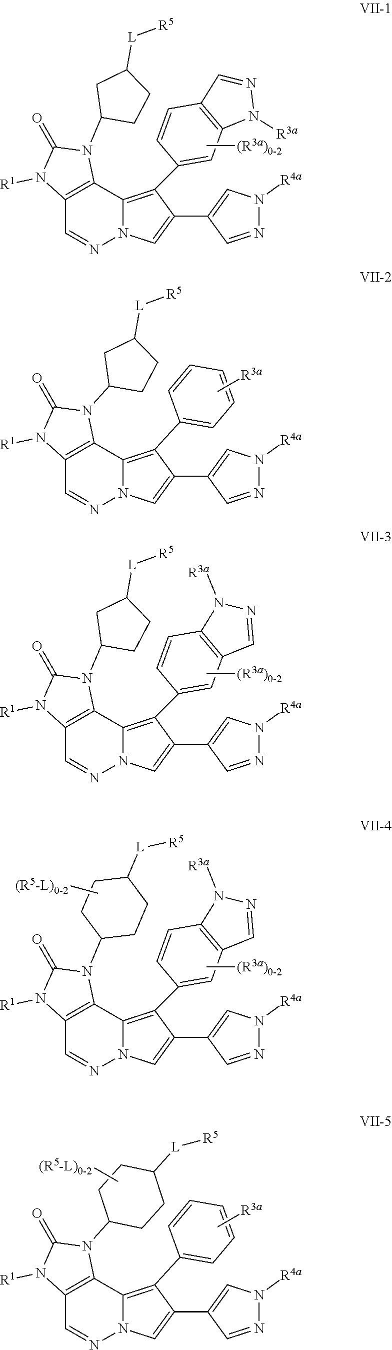
or a pharmaceutically acceptable salt thereof.

45. (canceled)

46. The compound of claim 1, wherein the compound is represented by formula IX: 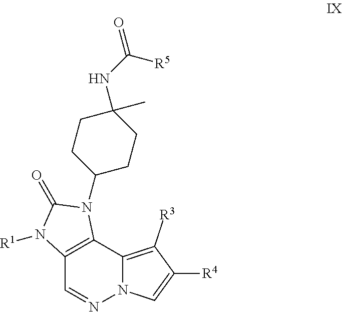
or a pharmaceutically acceptable salt thereof,
wherein R⁵ is optionally substituted cyclopropyl.

47. A compound selected from:

| No. | Structure |
|---|---|
| I-1 | 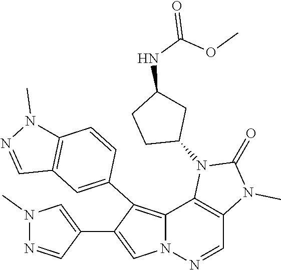 |
| I-1′ | 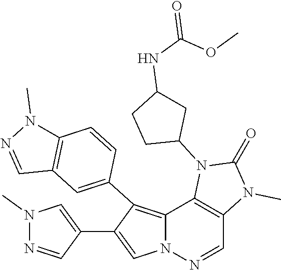 |
| I-2 | 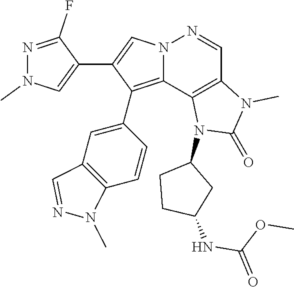 |
| I-2′ | 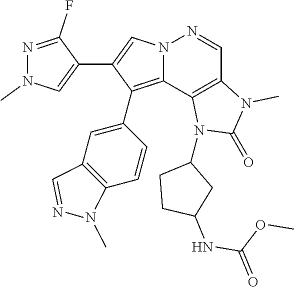 |
| I-3 | 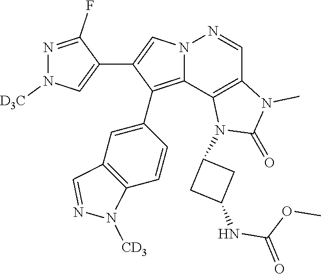 [SMILES: [2H]C([2H])([2H])n1cc(-c2cn3ncc4c(c3c2-c2ccc3c(cnn3C([2H])([2H])[2H])c2)n([C@H]2C[C@@H](NC(=O)OC)C2)c(=O)n4C)c(F)n1] |
| I-3′ | 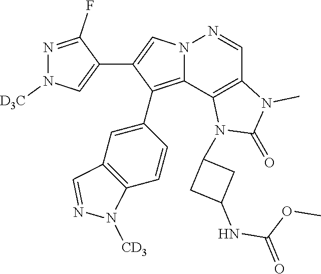 [SMILES: [2H]C([2H])([2H])n1cc(-c2cn3ncc4c(c3c2-c2ccc3c(cnn3C([2H])([2H])[2H])c2)n(C2CC(NC(=O)OC)C2)c(=O)n4C)c(F)n1] |
| I-4-1 | 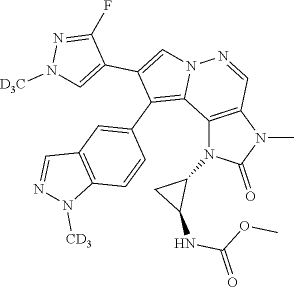 [SMILES: [2H]C([2H])([2H])n1cc(-c2cn3ncc4c(c3c2-c2ccc3c(cnn3C([2H])([2H])[2H])c2)n([C@H]2C[C@@H]2NC(=O)OC)c(=O)n4C)c(F)n1] |
| I-4-2 | 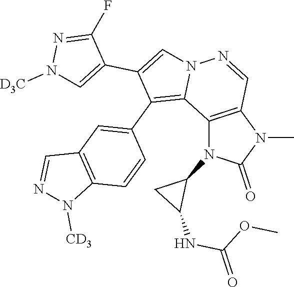 [SMILES: [2H]C([2H])([2H])n1cc(-c2cn3ncc4c(c3c2-c2ccc3c(cnn3C([2H])([2H])[2H])c2)n([C@@H]2C[C@H]2NC(=O)OC)c(=O)n4C)c(F)n1] |
| I-4′ | 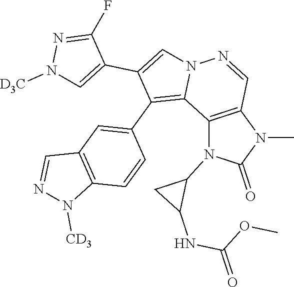 [SMILES: [2H]C([2H])([2H])n1cc(-c2cn3ncc4c(c3c2-c2ccc3c(cnn3C([2H])([2H])[2H])c2)n(C2CC2NC(=O)OC)c(=O)n4C)c(F)n1] |
| I-5 | 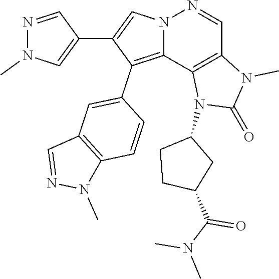 |
| I-5′ | 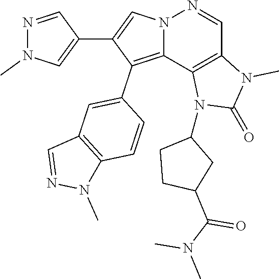 |
| I-6 | 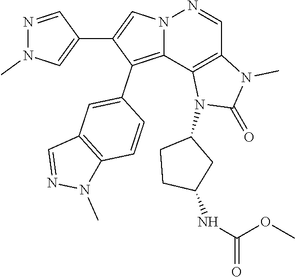 |
| I-6′ | 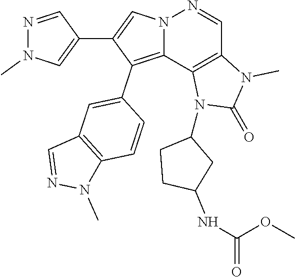 |
| I-7 | 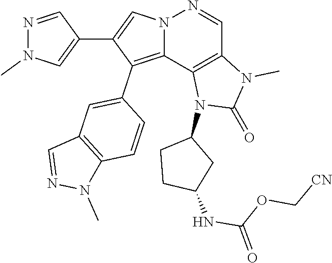 [SMILES: Cn1cc(-c2cn3ncc4c(c3c2-c2ccc3c(cnn3C)c2)n([C@@H]2CC[C@@H](NC(=O)OCC#N)C2)c(=O)n4C)cn1] |
| I-7′ | 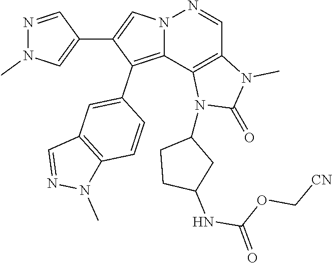 [SMILES: Cn1cc(-c2cn3ncc4c(c3c2-c2ccc3c(cnn3C)c2)n(C2CCC(NC(=O)OCC#N)C2)c(=O)n4C)cn1] |
| I-8 | 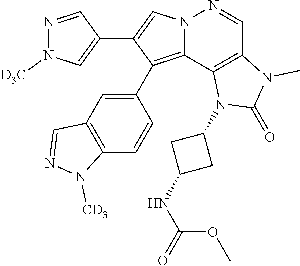 [SMILES: [2H]C([2H])([2H])n1cc(-c2cn3ncc4c(c3c2-c2ccc3c(cnn3C([2H])([2H])[2H])c2)n([C@H]2C[C@@H](NC(=O)OC)C2)c(=O)n4C)cn1] |
| I-8′ | 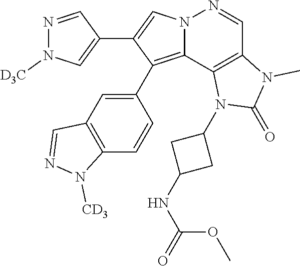 [SMILES: [2H]C([2H])([2H])n1cc(-c2cn3ncc4c(c3c2-c2ccc3c(cnn3C([2H])([2H])[2H])c2)n(C2CC(NC(=O)OC)C2)c(=O)n4C)cn1] |
| I-9 | 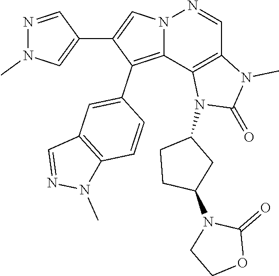 |
| I-9′ | 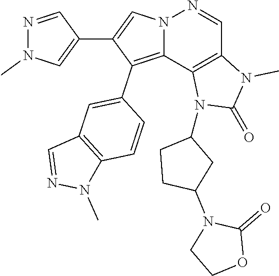 |
| I-10 | 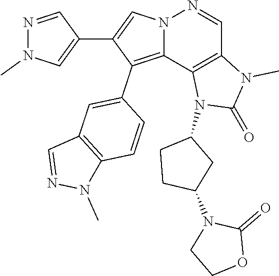 |
| I-10′ | 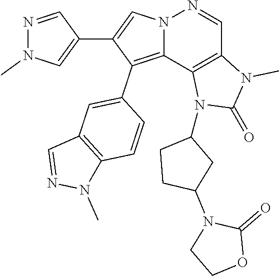 |
| I-11 | 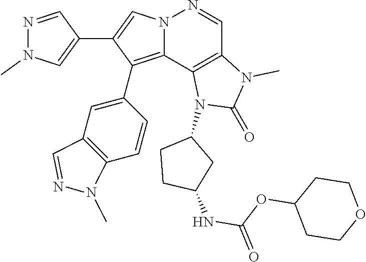 [SMILES: Cn1cc(-c2cn3ncc4c(c3c2-c2ccc3c(cnn3C)c2)n([C@H]2CC[C@@H](NC(=O)OC3CCOCC3)C2)c(=O)n4C)cn1] |
| I-11′ | 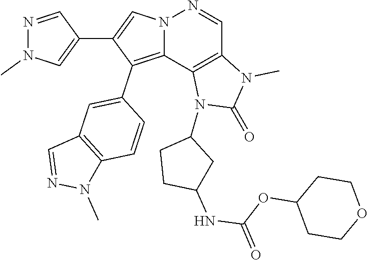 [SMILES: Cn1cc(-c2cn3ncc4c(c3c2-c2ccc3c(cnn3C)c2)n(C2CCC(NC(=O)OC3CCOCC3)C2)c(=O)n4C)cn1] |
| I-12 | 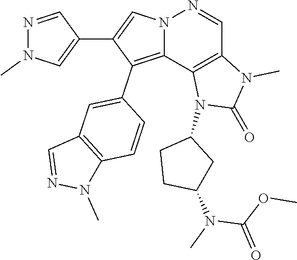 |
| I-12′ | 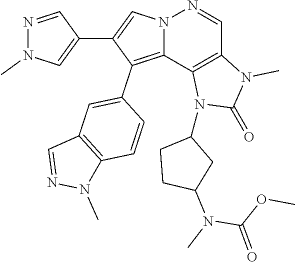 |
| I-13 | 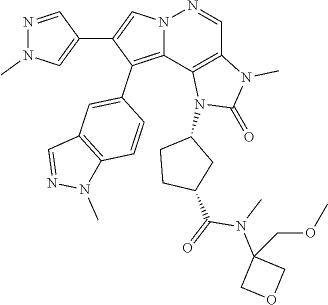 |
| I-13′ | 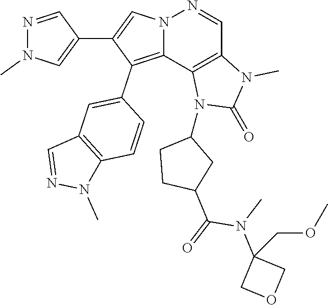 |
| I-14 | 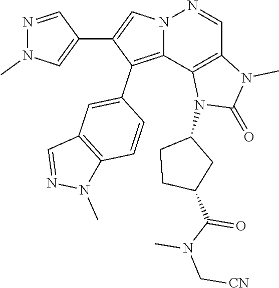 |
| I-14′ | 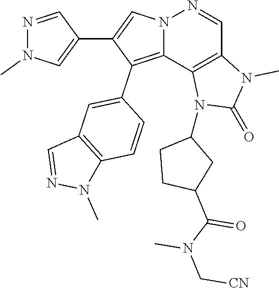 |
| I-15 | 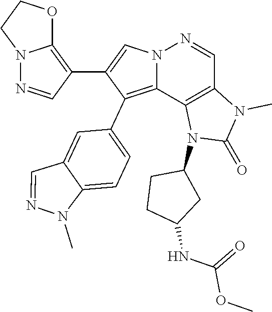 |
| I-15′ | 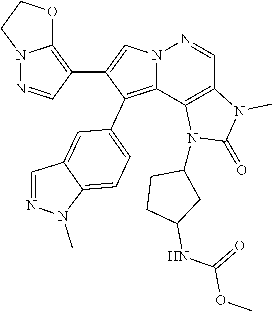 [SMILES: COC(=O)NC1CCC(n2c(=O)n(C)c3cnn4cc(-c5cnn6c5OCC6)c(-c5ccc6c(cnn6C)c5)c4c32)C1] |
| I-16 | 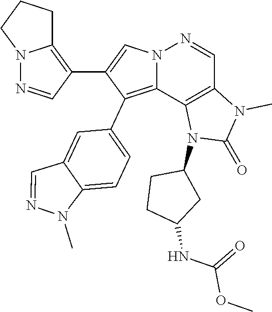 |
| I-16′ | 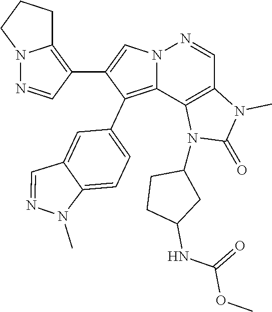 [SMILES: COC(=O)NC1CCC(n2c(=O)n(C)c3cnn4cc(-c5cnn6c5CCC6)c(-c5ccc6c(cnn6C)c5)c4c32)C1] |
| I-17 | 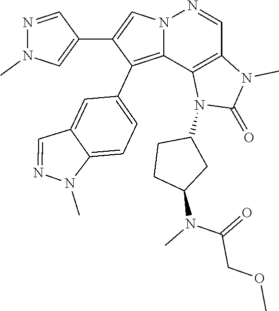 |
| I-17′ | 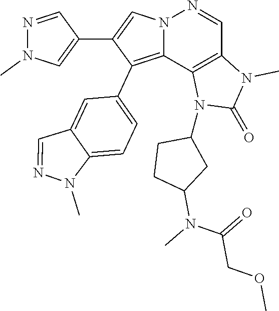 |
| I-18 | 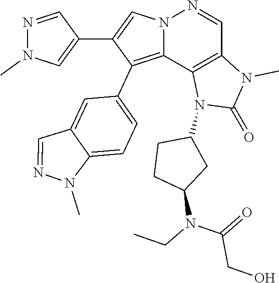 |
| I-18′ | 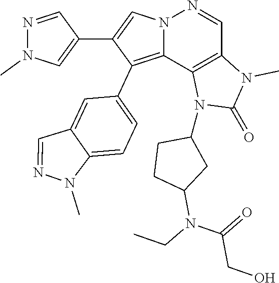 |
| I-19 | 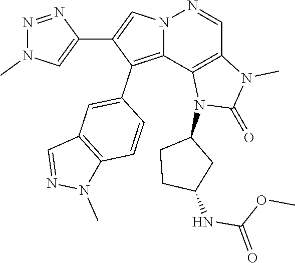 |
| I-19′ | 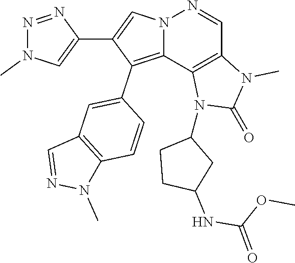 |
| I-20-1 | 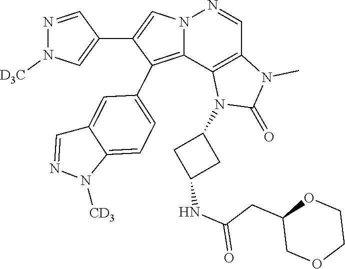 [SMILES: [2H]C([2H])([2H])n1cc(-c2cn3ncc4c(c3c2-c2ccc3c(cnn3C([2H])([2H])[2H])c2)n([C@H]2C[C@@H](NC(=O)C[C@@H]3COCCO3)C2)c(=O)n4C)cn1] |
| I-20-2 | 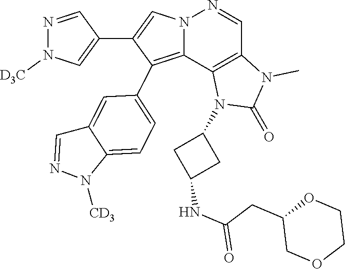 [SMILES: [2H]C([2H])([2H])n1cc(-c2cn3ncc4c(c3c2-c2ccc3c(cnn3C([2H])([2H])[2H])c2)n([C@H]2C[C@@H](NC(=O)C[C@H]3COCCO3)C2)c(=O)n4C)cn1] |
| I-20′ | 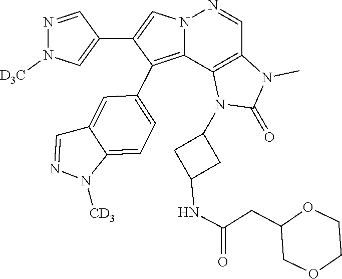 [SMILES: [2H]C([2H])([2H])n1cc(-c2cn3ncc4c(c3c2-c2ccc3c(cnn3C([2H])([2H])[2H])c2)n(C2CC(NC(=O)CC3COCCO3)C2)c(=O)n4C)cn1] |
| I-21 | 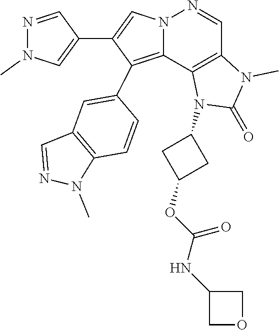 |
| I-21′ | 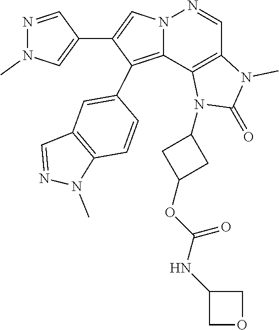 |
| I-22 | 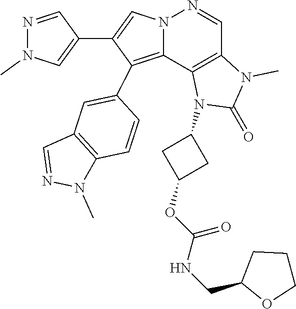 [SMILES: Cn1cc(-c2cn3ncc4c(c3c2-c2ccc3c(cnn3C)c2)n([C@H]2C[C@@H](OC(=O)NC[C@H]3CCCO3)C2)c(=O)n4C)cn1] |
| I-22′ | 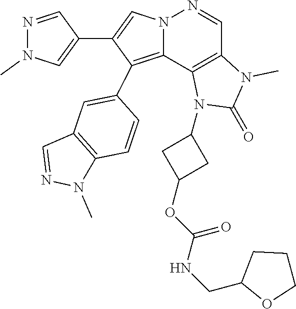 [SMILES: Cn1cc(-c2cn3ncc4c(c3c2-c2ccc3c(cnn3C)c2)n(C2CC(OC(=O)NCC3CCCO3)C2)c(=O)n4C)cn1] |
| I-23 | 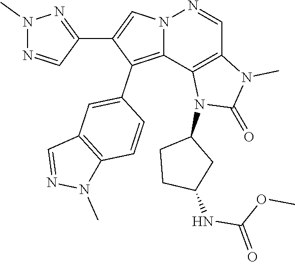 |
| I-23′ | 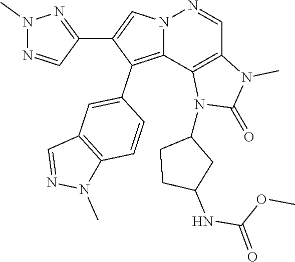 |
| I-24 | 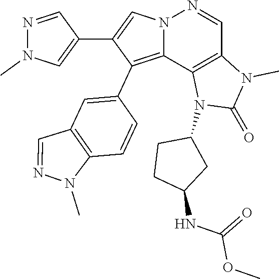 |
| I-24′ | 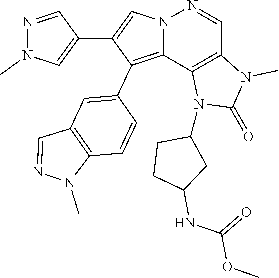 |
| I-25 | 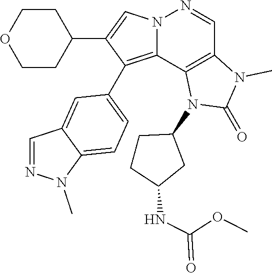 [SMILES: COC(=O)N[C@@H]1CC[C@@H](n2c(=O)n(C)c3cnn4cc(C5CCOCC5)c(-c5ccc6c(cnn6C)c5)c4c32)C1] |
| I-25′ | 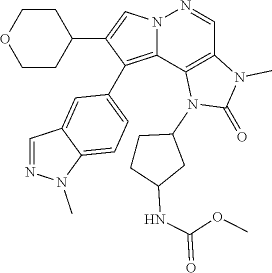 [SMILES: COC(=O)NC1CCC(n2c(=O)n(C)c3cnn4cc(C5CCOCC5)c(-c5ccc6c(cnn6C)c5)c4c32)C1] |
| I-26 | 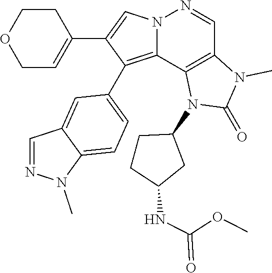 [SMILES: COC(=O)N[C@@H]1CC[C@@H](n2c(=O)n(C)c3cnn4cc(C5=CCOCC5)c(-c5ccc6c(cnn6C)c5)c4c32)C1] |
| I-26′ | 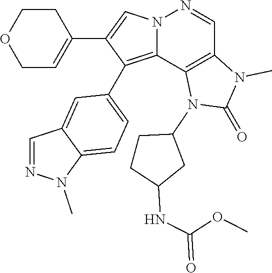 [SMILES: COC(=O)NC1CCC(n2c(=O)n(C)c3cnn4cc(C5=CCOCC5)c(-c5ccc6c(cnn6C)c5)c4c32)C1] |
| I-27 | 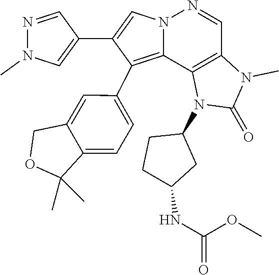 |
| I-27′ | 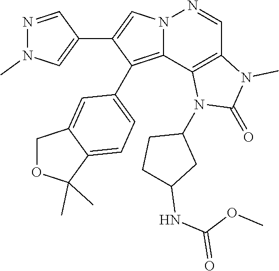 |
| I-28 | 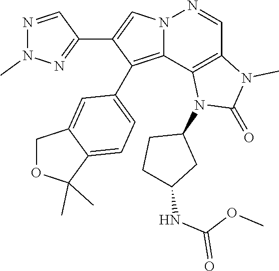 |
| I-28′ | 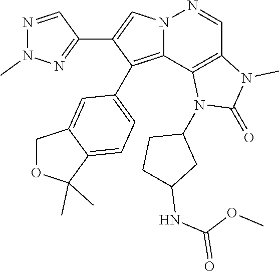 |
| I-29 | 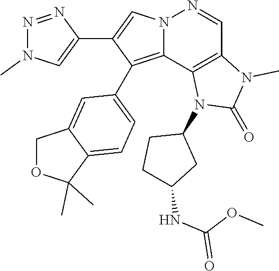 |
| I-29′ | 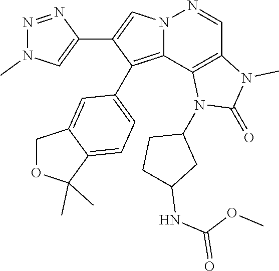 |
| I-30 | 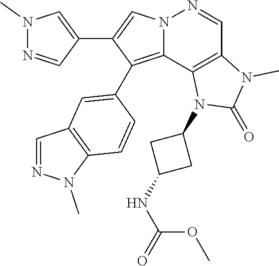 |
| I-30′ | 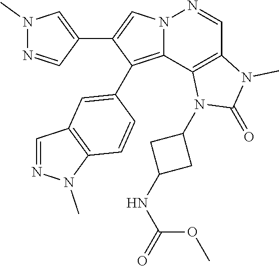 |
| I-31 | 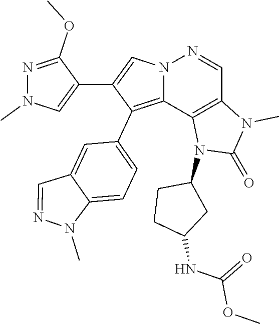 |
| I-31′ | 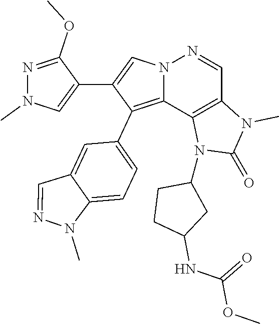 |
| I-32 | 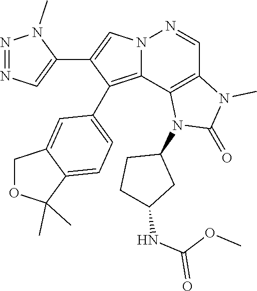 [SMILES: COC(=O)N[C@@H]1CC[C@@H](n2c(=O)n(C)c3cnn4cc(-c5cnnn5C)c(-c5ccc6c(c5)COC6(C)C)c4c32)C1] |
| I-32′ | 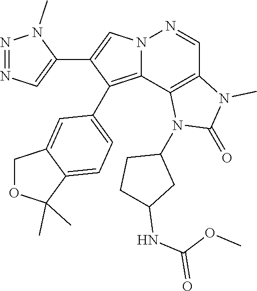 [SMILES: COC(=O)NC1CCC(n2c(=O)n(C)c3cnn4cc(-c5cnnn5C)c(-c5ccc6c(c5)COC6(C)C)c4c32)C1] |
| I-33 | 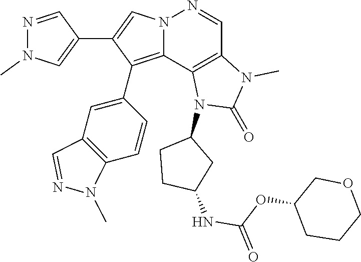 [SMILES: Cn1cc(-c2cn3ncc4c(c3c2-c2ccc3c(cnn3C)c2)n([C@@H]2CC[C@@H](NC(=O)O[C@H]3CCCOC3)C2)c(=O)n4C)cn1] |
| I-33′ | 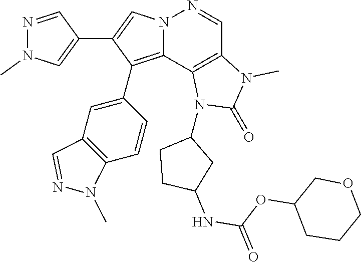 [SMILES: Cn1cc(-c2cn3ncc4c(c3c2-c2ccc3c(cnn3C)c2)n(C2CCC(NC(=O)OC3CCCOC3)C2)c(=O)n4C)cn1] |
| I-34 | 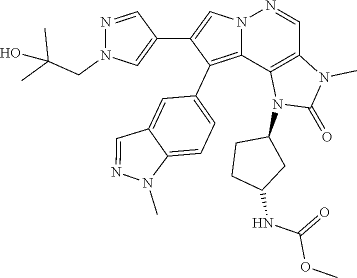 |
| I-34′ | 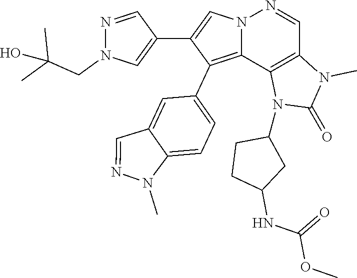 [SMILES: COC(=O)NC1CCC(n2c(=O)n(C)c3cnn4cc(-c5cnn(CC(C)(C)O)c5)c(-c5ccc6c(cnn6C)c5)c4c32)C1] |
| I-35 | 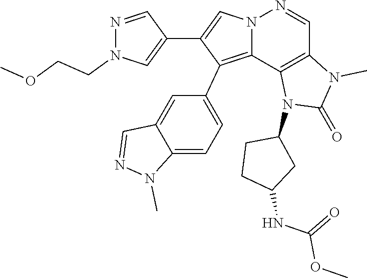 |
| I-35′ | 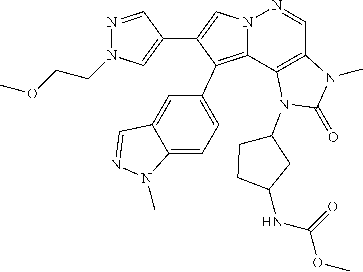 |
| I-36 | 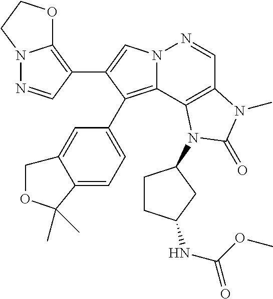 [SMILES: COC(=O)N[C@@H]1CC[C@@H](n2c(=O)n(C)c3cnn4cc(-c5cnn6c5OCC6)c(-c5ccc6c(c5)COC6(C)C)c4c32)C1] |
| I-36′ | 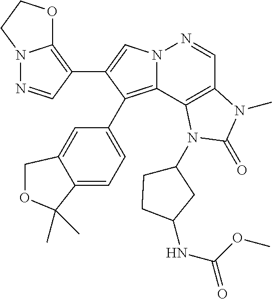 [SMILES: COC(=O)NC1CCC(n2c(=O)n(C)c3cnn4cc(-c5cnn6c5OCC6)c(-c5ccc6c(c5)COC6(C)C)c4c32)C1] |
| I-37-1 | 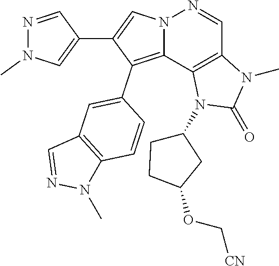 |
| I-37-2 | 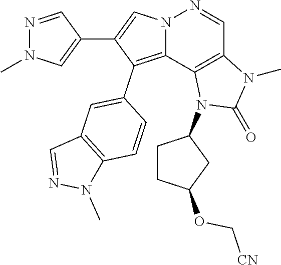 |
| I-37′ | 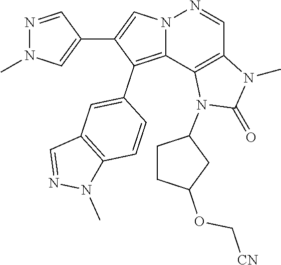 |
| I-38 | 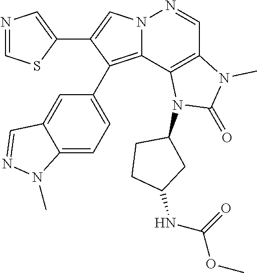 |
| I-38′ | 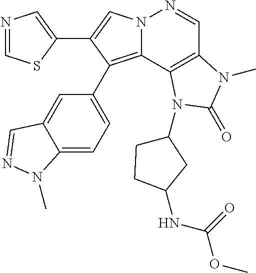 [SMILES: COC(=O)NC1CCC(n2c(=O)n(C)c3cnn4cc(-c5cncs5)c(-c5ccc6c(cnn6C)c5)c4c32)C1] |
| I-40 | 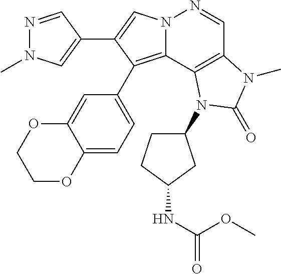 |
| I-40′ | 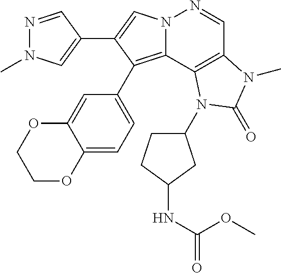 |
| I-41 | 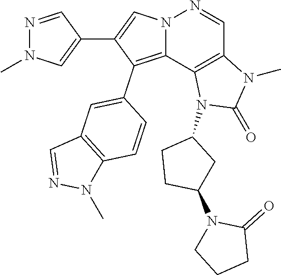 |
| I-41′ | 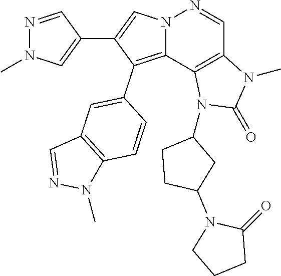 |
| I-42 | 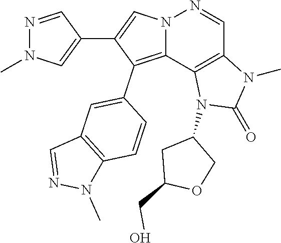 |
| I-42′ | 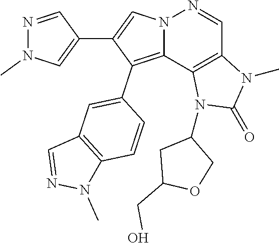 |
| I-43 | 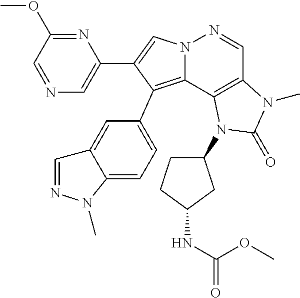 |
| I-43′ | 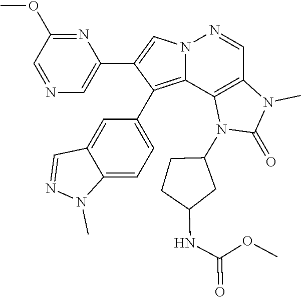 |
| I-44 | 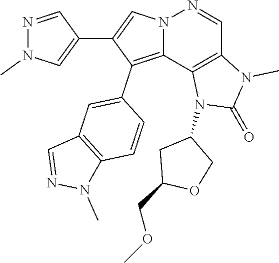 |
| I-44′ | 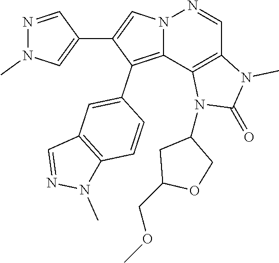 |
| I-45 | 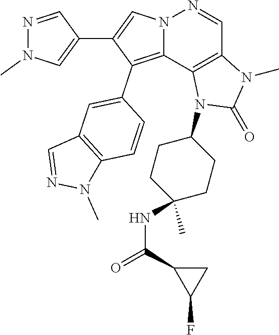 |
| I-45′ | 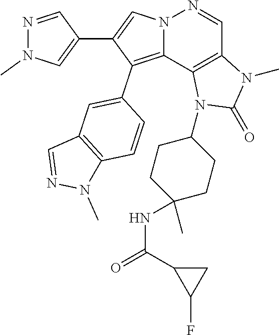 |
| I-46 | 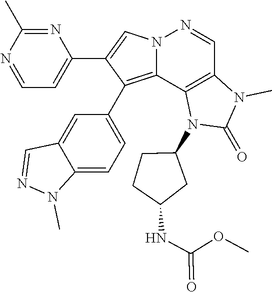 [SMILES: COC(=O)N[C@@H]1CC[C@@H](n2c(=O)n(C)c3cnn4cc(-c5ccnc(C)n5)c(-c5ccc6c(cnn6C)c5)c4c32)C1] |
| I-46′ | 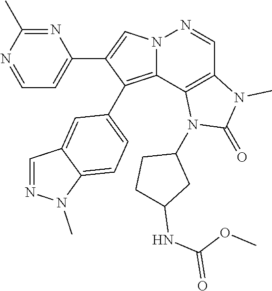 [SMILES: COC(=O)NC1CCC(n2c(=O)n(C)c3cnn4cc(-c5ccnc(C)n5)c(-c5ccc6c(cnn6C)c5)c4c32)C1] |
| I-47 | 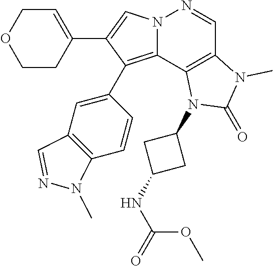 [SMILES: COC(=O)N[C@H]1C[C@H](n2c(=O)n(C)c3cnn4cc(C5=CCOCC5)c(-c5ccc6c(cnn6C)c5)c4c32)C1] |
| I-47′ | 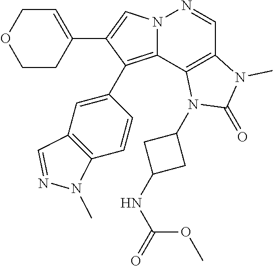 [SMILES: COC(=O)NC1CC(n2c(=O)n(C)c3cnn4cc(C5=CCOCC5)c(-c5ccc6c(cnn6C)c5)c4c32)C1] |
| I-48 | 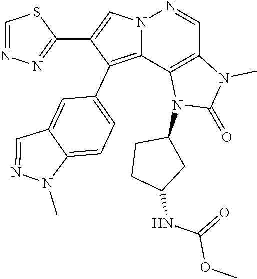 |
| I-48′ | 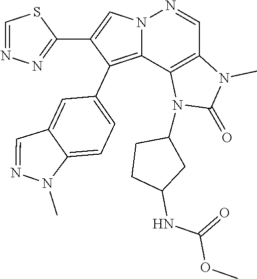 [SMILES: COC(=O)NC1CCC(n2c(=O)n(C)c3cnn4cc(-c5nncs5)c(-c5ccc6c(cnn6C)c5)c4c32)C1] |
| I-49 | 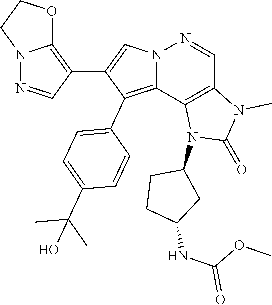 [SMILES: COC(=O)N[C@@H]1CC[C@@H](n2c(=O)n(C)c3cnn4cc(-c5cnn6c5OCC6)c(-c5ccc(C(C)(C)O)cc5)c4c32)C1] |
| I-49′ | 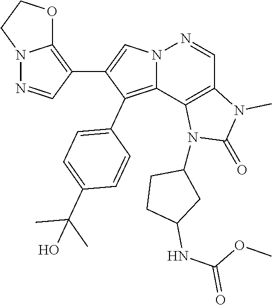 [SMILES: COC(=O)NC1CCC(n2c(=O)n(C)c3cnn4cc(-c5cnn6c5OCC6)c(-c5ccc(C(C)(C)O)cc5)c4c32)C1] |
| I-50 | 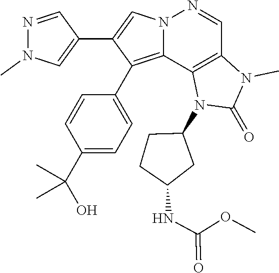 |
| I-50′ | 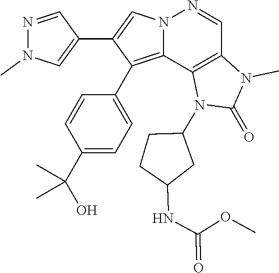 |
| I-51 | 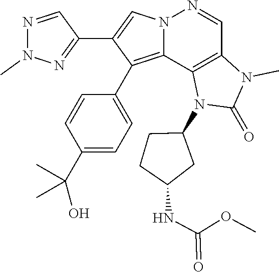 |
| I-51′ | 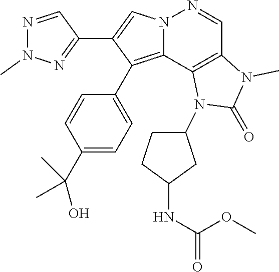 |
| I-52 | 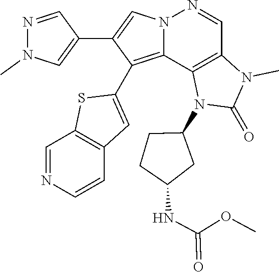 |
| I-52′ | 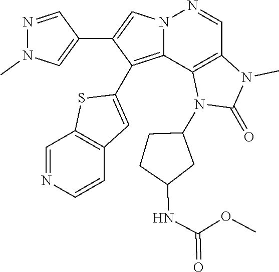 |
| I-53 | 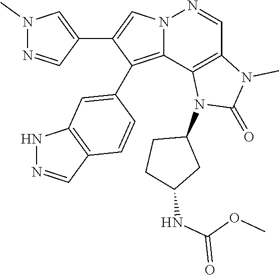 |
| I-53′ | 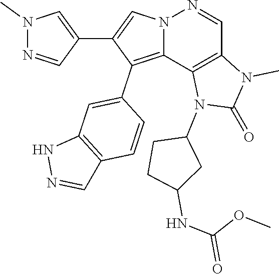 |
| I-54 | 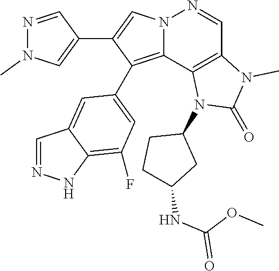 |
| I-54′ | 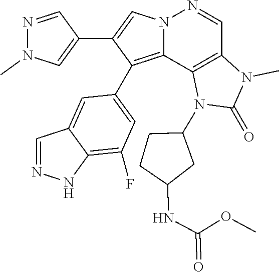 |
| I-55 | 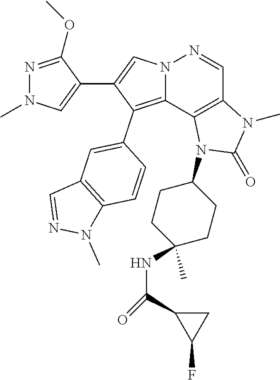 |
| I-55′ | 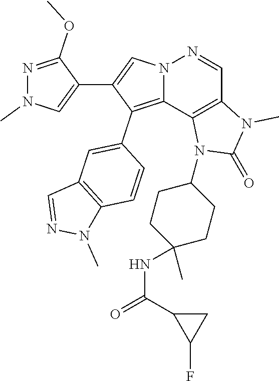 |
| I-56 | 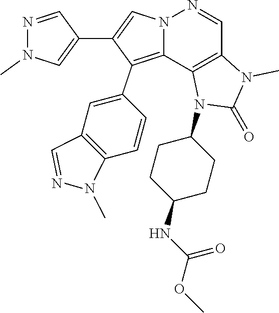 |
| I-56′ | 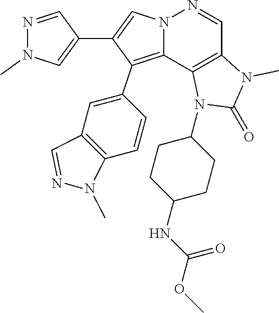 |
| I-57 | 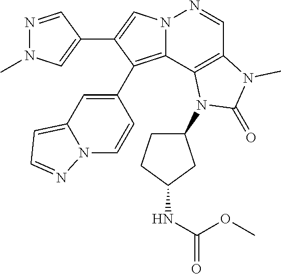 |
| I-57′ | 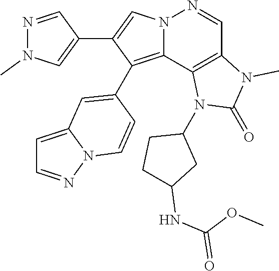 |
| I-58 | 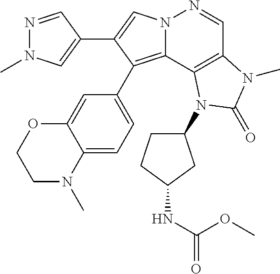 |
| I-58′ | 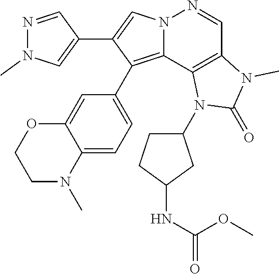 |
| I-59 | 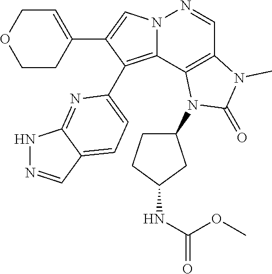 [SMILES: COC(=O)N[C@@H]1CC[C@@H](n2c(=O)n(C)c3cnn4cc(C5=CCOCC5)c(-c5ccc6cn[nH]c6n5)c4c32)C1] |
| I-59′ | 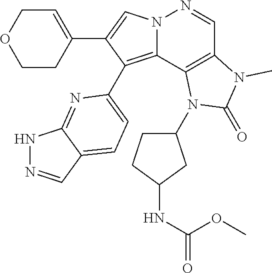 [SMILES: COC(=O)NC1CCC(n2c(=O)n(C)c3cnn4cc(C5=CCOCC5)c(-c5ccc6cn[nH]c6n5)c4c32)C1] |
| I-60 | 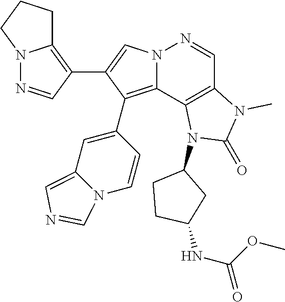 [SMILES: COC(=O)N[C@@H]1CC[C@@H](n2c(=O)n(C)c3cnn4cc(-c5cnn6c5CCC6)c(-c5ccn6cncc6c5)c4c32)C1] |
| I-60′ | 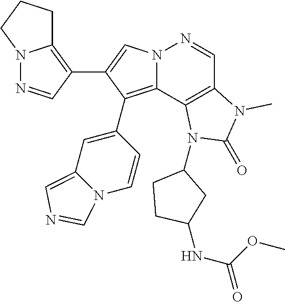 [SMILES: COC(=O)NC1CCC(n2c(=O)n(C)c3cnn4cc(-c5cnn6c5CCC6)c(-c5ccn6cncc6c5)c4c32)C1] |
| I-61 | 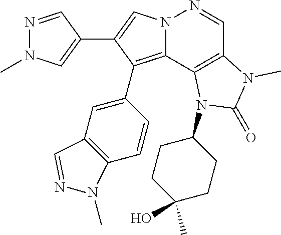 |
| I-61′ | 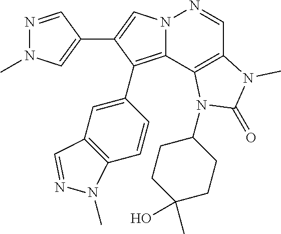 |
| I-62 | 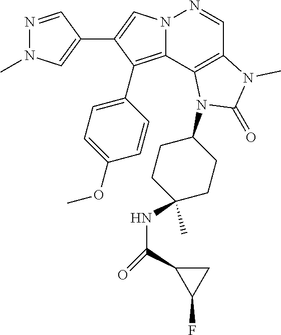 |
| I-62′ | 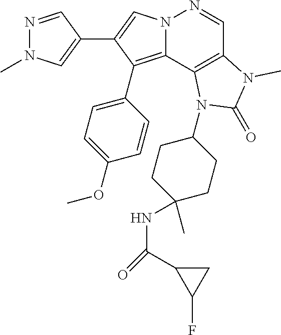 |
| I-63 | 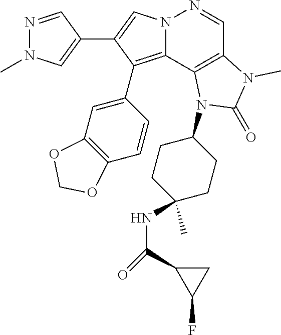 |
| I-63′ | 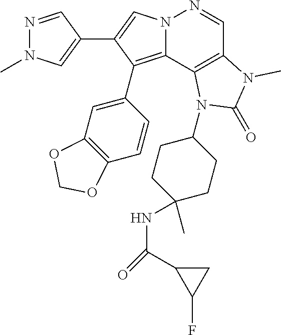 |
| I-64 | 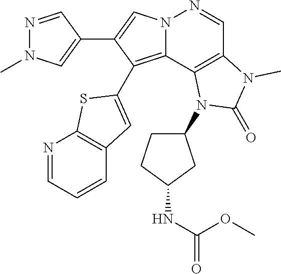 |
| I-64′ | 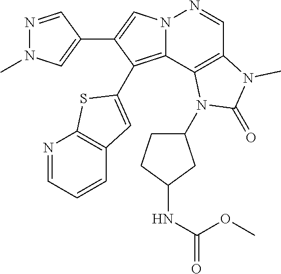 |
| I-65 | 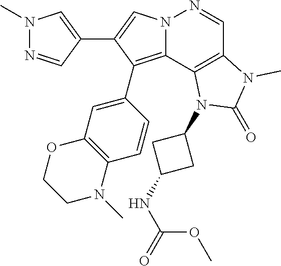 |
| I-65′ | 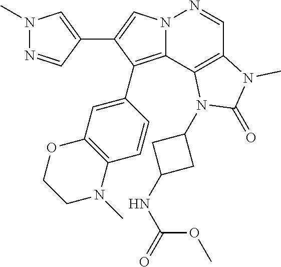 |
| I-66 | 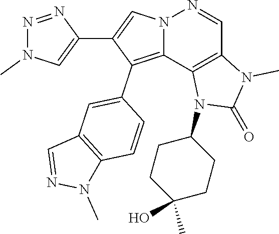 |
| I-66′ | 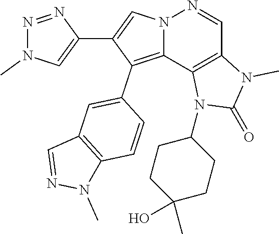 |
| I-67 | 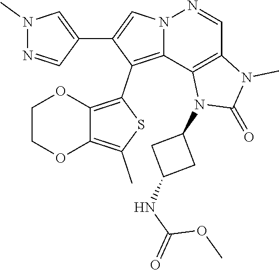 |
| I-67′ | 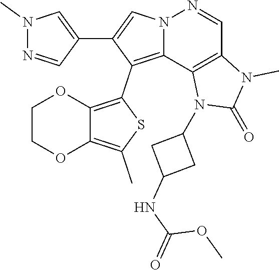 |
| I-68 | 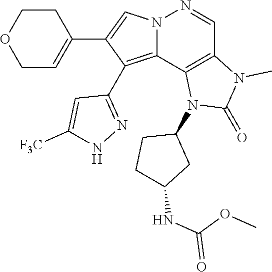 [SMILES: COC(=O)N[C@@H]1CC[C@@H](n2c(=O)n(C)c3cnn4cc(C5=CCOCC5)c(-c5cc(C(F)(F)F)[nH]n5)c4c32)C1] |
| I-68′ | 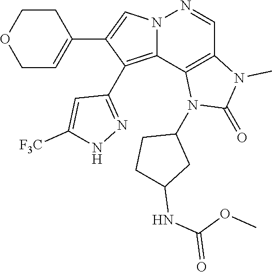 [SMILES: COC(=O)NC1CCC(n2c(=O)n(C)c3cnn4cc(C5=CCOCC5)c(-c5cc(C(F)(F)F)[nH]n5)c4c32)C1] |
| I-69 | 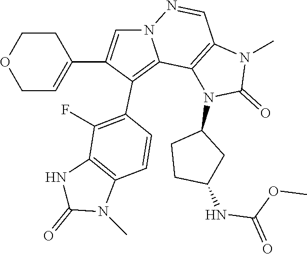 [SMILES: COC(=O)N[C@@H]1CC[C@@H](n2c(=O)n(C)c3cnn4cc(C5=CCOCC5)c(-c5ccc6c([nH]c(=O)n6C)c5F)c4c32)C1] |
| I-69′ | 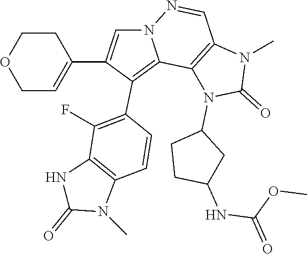 [SMILES: COC(=O)NC1CCC(n2c(=O)n(C)c3cnn4cc(C5=CCOCC5)c(-c5ccc6c([nH]c(=O)n6C)c5F)c4c32)C1] |
| I-70 | 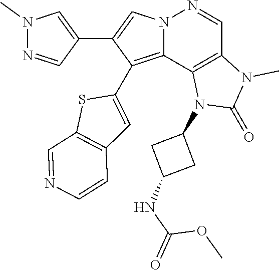 |
| I-70′ | 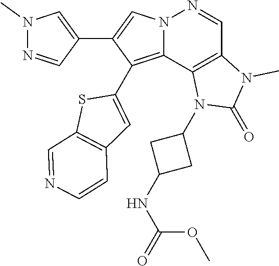 |
| I-71 | 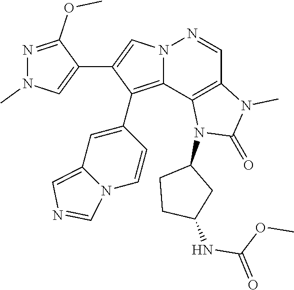 |
| I-71′ | 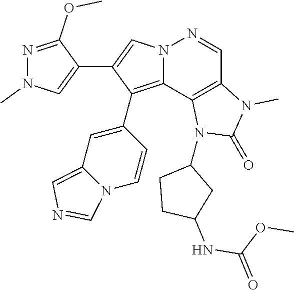 |
| I-72 | 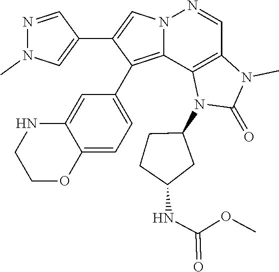 |
| I-72′ | 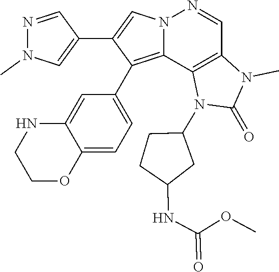 |
| I-73 | 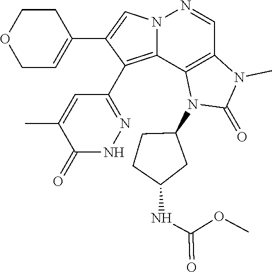 [SMILES: COC(=O)N[C@@H]1CC[C@@H](n2c(=O)n(C)c3cnn4cc(C5=CCOCC5)c(-c5cc(C)c(=O)[nH]n5)c4c32)C1] |
| I-73′ | 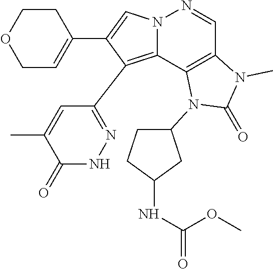 [SMILES: COC(=O)NC1CCC(n2c(=O)n(C)c3cnn4cc(C5=CCOCC5)c(-c5cc(C)c(=O)[nH]n5)c4c32)C1] |
| I-74 | 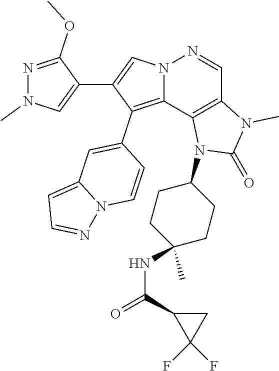 |
| I-74′ | 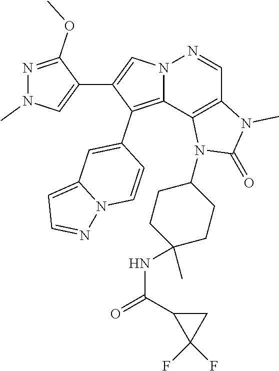 |
| I-75 | 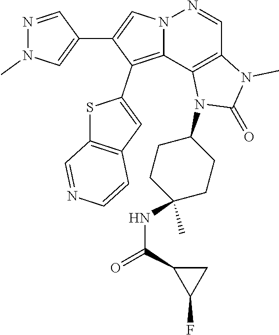 |
| I-75′ | 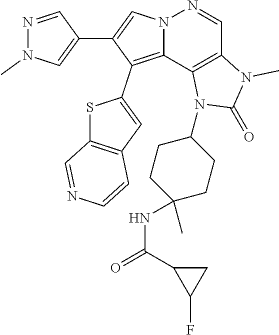 |
| I-76 | 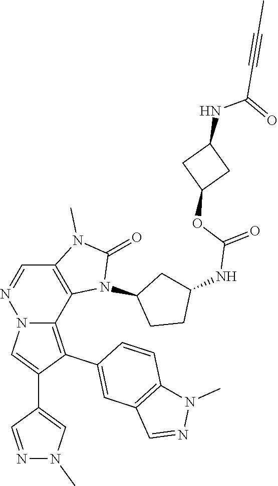 [SMILES: CC#CC(=O)N[C@H]1C[C@@H](OC(=O)N[C@@H]2CC[C@@H](n3c(=O)n(C)c4cnn5cc(-c6cnn(C)c6)c(-c6ccc7c(cnn7C)c6)c5c43)C2)C1] |
| I-76′ | 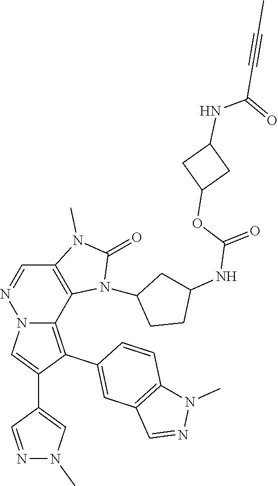 [SMILES: CC#CC(=O)NC1CC(OC(=O)NC2CCC(n3c(=O)n(C)c4cnn5cc(-c6cnn(C)c6)c(-c6ccc7c(cnn7C)c6)c5c43)C2)C1] |
| I-77 | 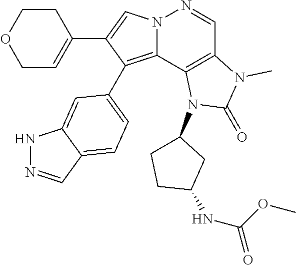 [SMILES: COC(=O)N[C@@H]1CC[C@@H](n2c(=O)n(C)c3cnn4cc(C5=CCOCC5)c(-c5ccc6cn[nH]c6c5)c4c32)C1] |
| I-77′ | 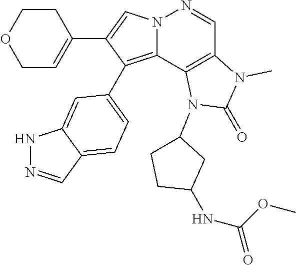 [SMILES: COC(=O)NC1CCC(n2c(=O)n(C)c3cnn4cc(C5=CCOCC5)c(-c5ccc6cn[nH]c6c5)c4c32)C1] |
| I-78 | 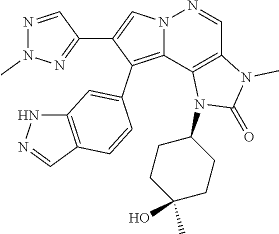 |
| I-78′ | 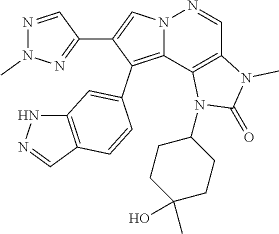 |
| I-79 | 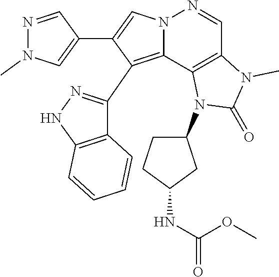 |
| I-79′ | 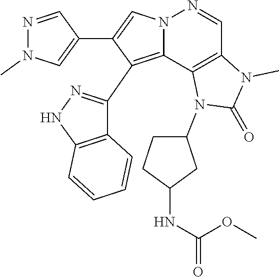 |
| I-80 | 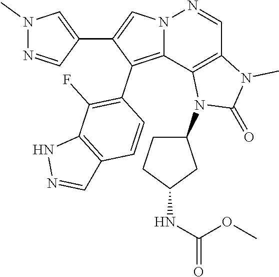 |
| I-80′ | 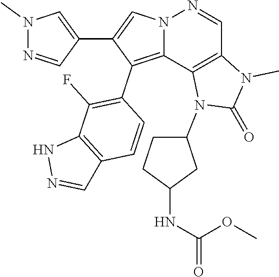 |
| I-81 | 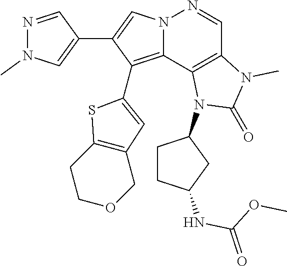 |
| I-81′ | 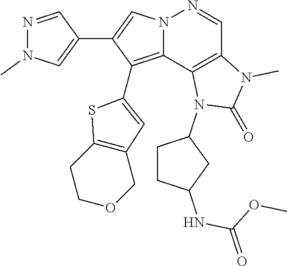 |
| I-82 | 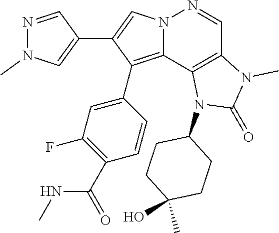 |
| I-82′ | 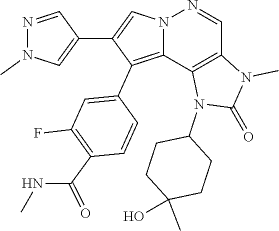 |
| I-83 | 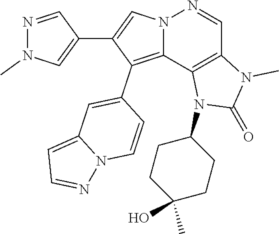 |
| I-83′ | 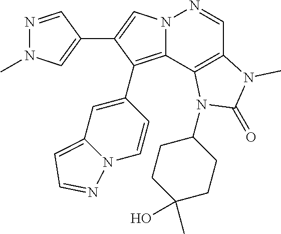 |
| I-84 | 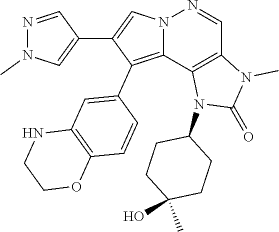 |
| I-84′ | 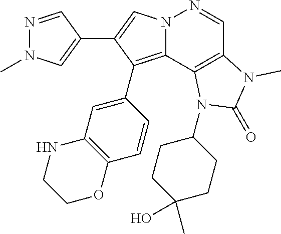 |
| I-85 | 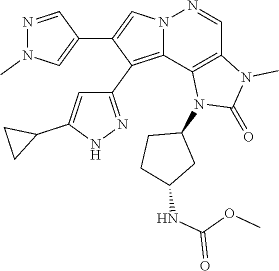 |
| I-85′ | 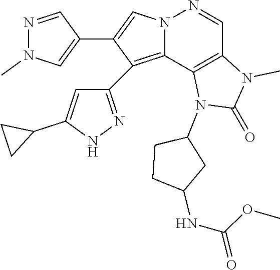 [SMILES: COC(=O)NC1CCC(n2c(=O)n(C)c3cnn4cc(-c5cnn(C)c5)c(-c5cc(C6CC6)[nH]n5)c4c32)C1] |
| I-86 | 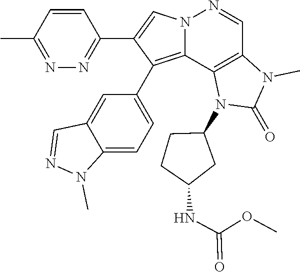 |
| I-86′ | 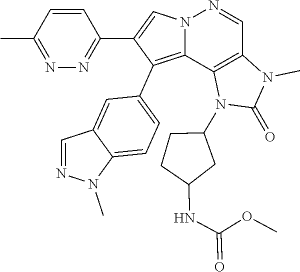 |
| I-87 | 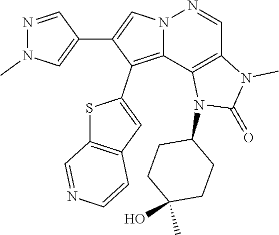 |
| I-87′ | 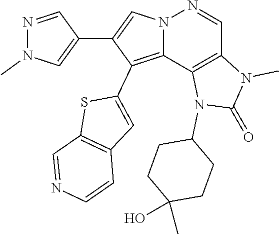 |
| I-88 | 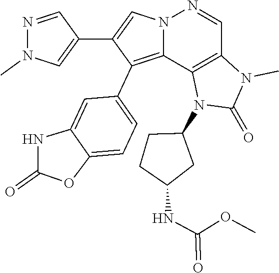 |
| I-88′ | 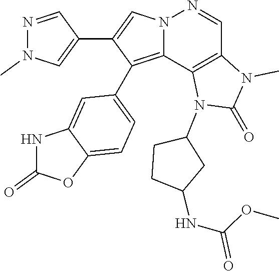 |
| I-89 | 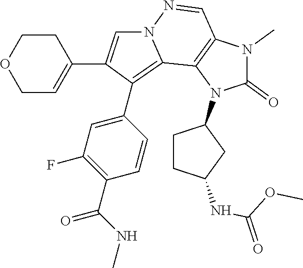 [SMILES: CNC(=O)c1ccc(-c2c(C3=CCOCC3)cn3ncc4c(c23)n([C@@H]2CC[C@@H](NC(=O)OC)C2)c(=O)n4C)cc1F] |
| I-89′ | 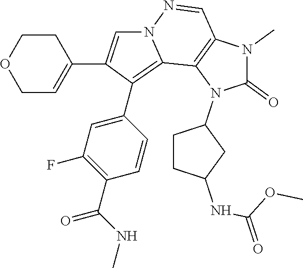 [SMILES: CNC(=O)c1ccc(-c2c(C3=CCOCC3)cn3ncc4c(c23)n(C2CCC(NC(=O)OC)C2)c(=O)n4C)cc1F] |
| I-90 | 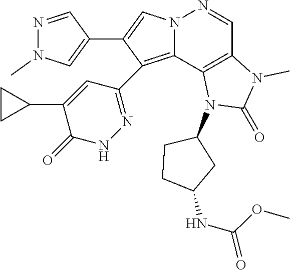 [SMILES: COC(=O)N[C@@H]1CC[C@@H](n2c(=O)n(C)c3cnn4cc(-c5cnn(C)c5)c(-c5cc(C6CC6)c(=O)[nH]n5)c4c32)C1] |
| I-90′ | 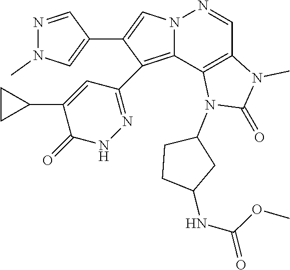 [SMILES: COC(=O)NC1CCC(n2c(=O)n(C)c3cnn4cc(-c5cnn(C)c5)c(-c5cc(C6CC6)c(=O)[nH]n5)c4c32)C1] |
| I-91 | 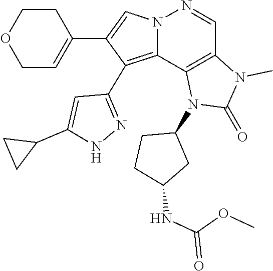 [SMILES: COC(=O)N[C@@H]1CC[C@@H](n2c(=O)n(C)c3cnn4cc(C5=CCOCC5)c(-c5cc(C6CC6)[nH]n5)c4c32)C1] |
| I-91′ | 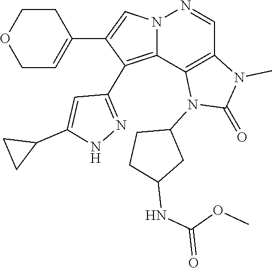 [SMILES: COC(=O)NC1CCC(n2c(=O)n(C)c3cnn4cc(C5=CCOCC5)c(-c5cc(C6CC6)[nH]n5)c4c32)C1] |
| I-93 | 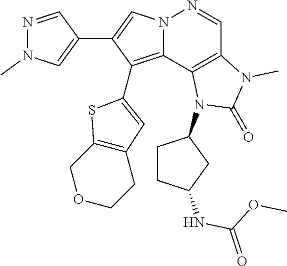 |
| I-93′ | 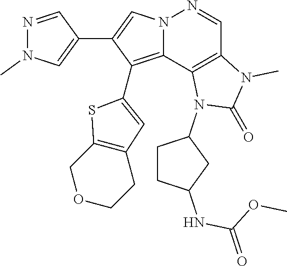 |
| I-94 | 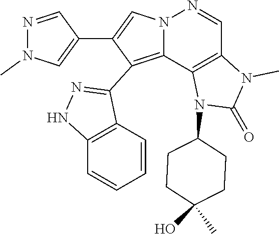 |
| I-94′ | 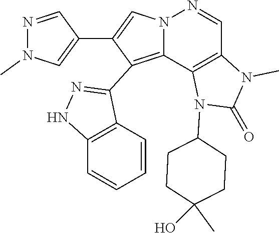 |
| I-95 | 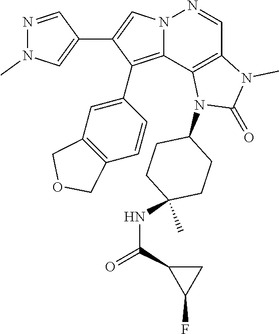 |
| I-95′ | 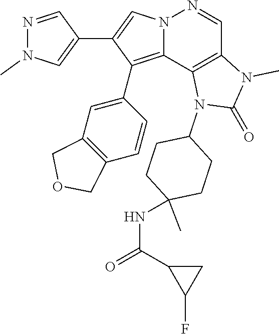 |
| I-96 | 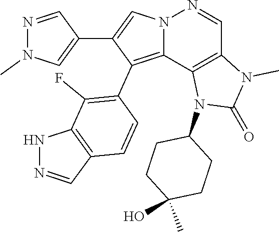 |
| I-96′ | 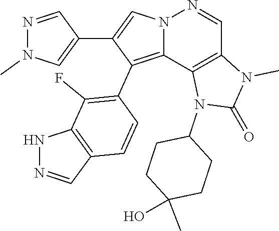 |
| I-97 | 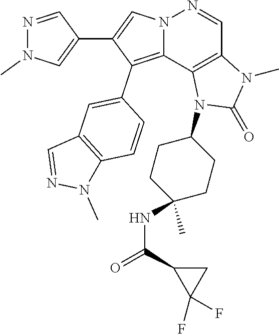 |
| I-97′ | 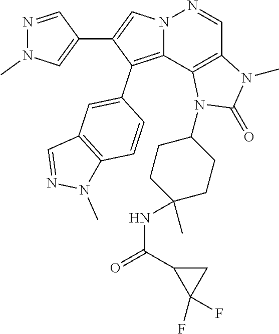 |
| I-98 | 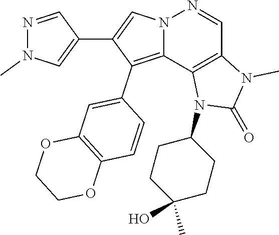 |
| I-98′ | 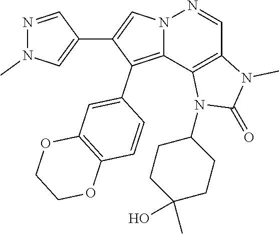 |
| I-99 | 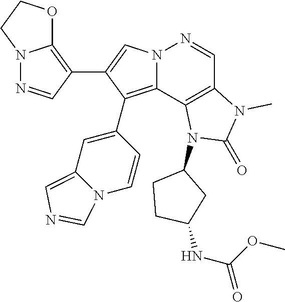 [SMILES: COC(=O)N[C@@H]1CC[C@@H](n2c(=O)n(C)c3cnn4cc(-c5cnn6c5OCC6)c(-c5ccn6cncc6c5)c4c32)C1] |
| I-99′ | 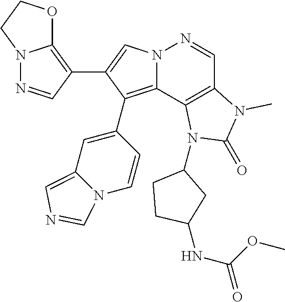 [SMILES: COC(=O)NC1CCC(n2c(=O)n(C)c3cnn4cc(-c5cnn6c5OCC6)c(-c5ccn6cncc6c5)c4c32)C1] |
| I-100 | 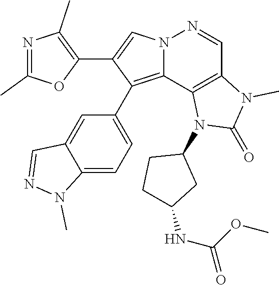 |
| I-100′ | 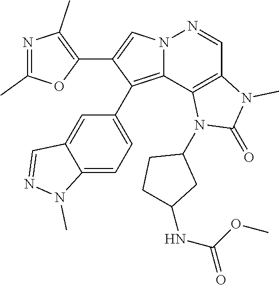 |
| I-101 | 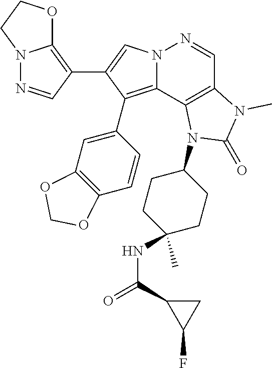 [SMILES: Cn1c(=O)n([C@H]2CC[C@@](C)(NC(=O)[C@H]3C[C@H]3F)CC2)c2c1cnn1cc(-c3cnn4c3OCC4)c(-c3ccc4c(c3)OCO4)c21] |
| I-101′ | 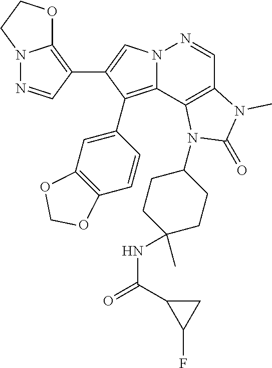 [SMILES: Cn1c(=O)n(C2CCC(C)(NC(=O)C3CC3F)CC2)c2c1cnn1cc(-c3cnn4c3OCC4)c(-c3ccc4c(c3)OCO4)c21] |
| I-102 | 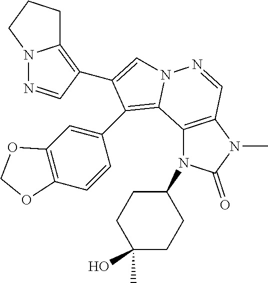 [SMILES: Cn1c(=O)n([C@H]2CC[C@@](C)(O)CC2)c2c1cnn1cc(-c3cnn4c3CCC4)c(-c3ccc4c(c3)OCO4)c21] |
| I-102′ | 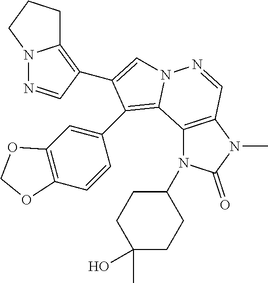 [SMILES: Cn1c(=O)n(C2CCC(C)(O)CC2)c2c1cnn1cc(-c3cnn4c3CCC4)c(-c3ccc4c(c3)OCO4)c21] |
| I-103 | 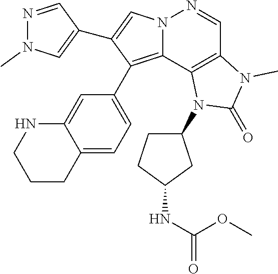 |
| I-103′ | 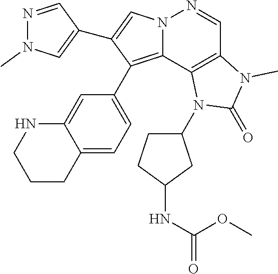 |
| I-104 | 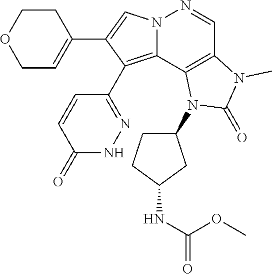 [SMILES: COC(=O)N[C@@H]1CC[C@@H](n2c(=O)n(C)c3cnn4cc(C5=CCOCC5)c(-c5ccc(=O)[nH]n5)c4c32)C1] |
| I-104′ | 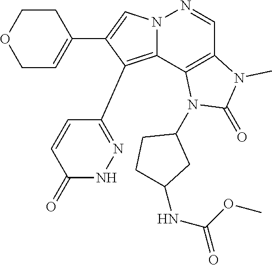 [SMILES: COC(=O)NC1CCC(n2c(=O)n(C)c3cnn4cc(C5=CCOCC5)c(-c5ccc(=O)[nH]n5)c4c32)C1] |
| I-105 | 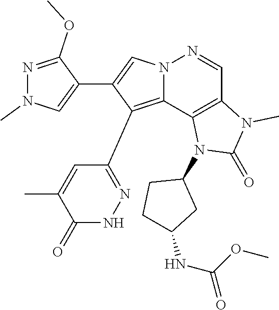 |
| I-105′ | 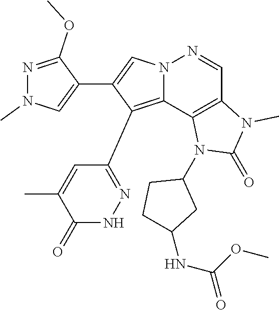 |
| I-106 | 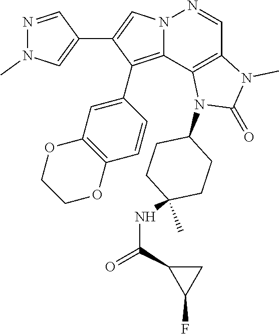 |
| I-106′ | 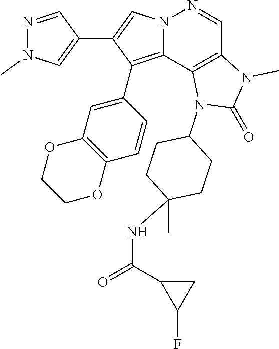 |
| I-107 | 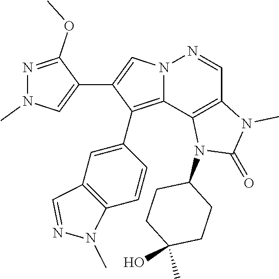 |
| I-107′ | 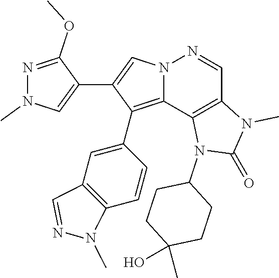 |
| I-108 | 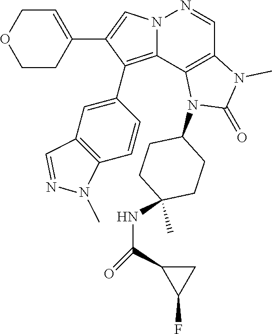 [SMILES: Cn1ncc2cc(-c3c(C4=CCOCC4)cn4ncc5c(c34)n([C@H]3CC[C@@](C)(NC(=O)[C@H]4C[C@H]4F)CC3)c(=O)n5C)ccc21] |
| I-108′ | 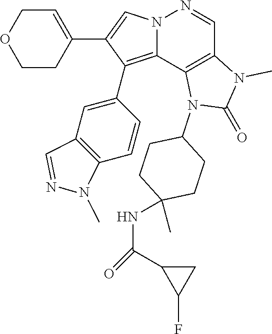 [SMILES: Cn1ncc2cc(-c3c(C4=CCOCC4)cn4ncc5c(c34)n(C3CCC(C)(NC(=O)C4CC4F)CC3)c(=O)n5C)ccc21] |
| I-109 | 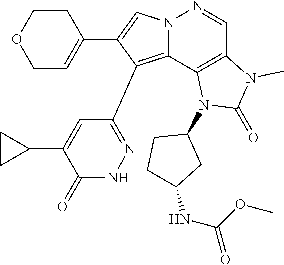 [SMILES: COC(=O)N[C@@H]1CC[C@@H](n2c(=O)n(C)c3cnn4cc(C5=CCOCC5)c(-c5cc(C6CC6)c(=O)[nH]n5)c4c32)C1] |
| I-109′ | 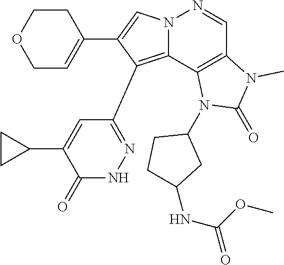 [SMILES: COC(=O)NC1CCC(n2c(=O)n(C)c3cnn4cc(C5=CCOCC5)c(-c5cc(C6CC6)c(=O)[nH]n5)c4c32)C1] |
| I-110 | 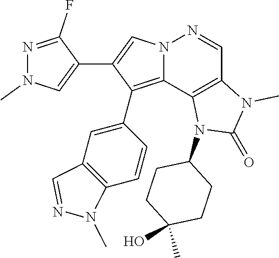 |
| I-110′ | 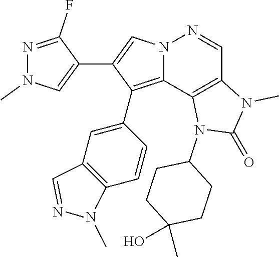 |
| I-111 | 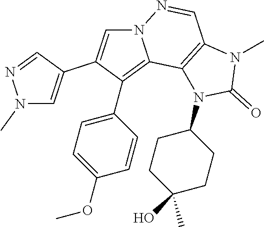 |
| I-111′ | 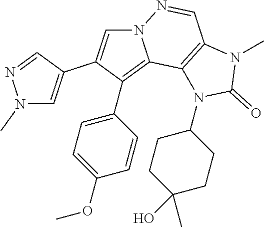 |
| I-112 | 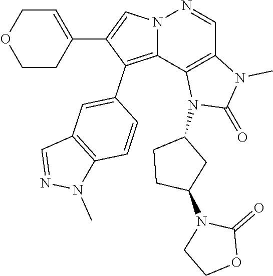 [SMILES: Cn1ncc2cc(-c3c(C4=CCOCC4)cn4ncc5c(c34)n([C@H]3CC[C@H](N4CCOC4=O)C3)c(=O)n5C)ccc21] |
| I-112′ | 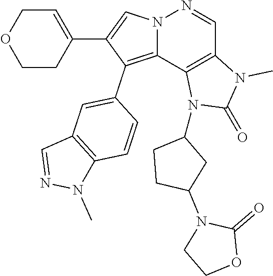 [SMILES: Cn1ncc2cc(-c3c(C4=CCOCC4)cn4ncc5c(c34)n(C3CCC(N4CCOC4=O)C3)c(=O)n5C)ccc21] |
| I-113 | 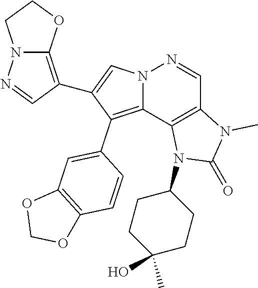 [SMILES: Cn1c(=O)n([C@H]2CC[C@@](C)(O)CC2)c2c1cnn1cc(-c3cnn4c3OCC4)c(-c3ccc4c(c3)OCO4)c21] |
| I-113′ | 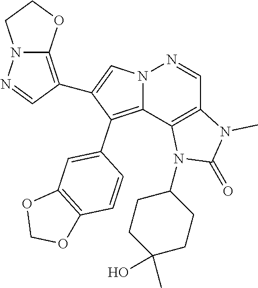 [SMILES: Cn1c(=O)n(C2CCC(C)(O)CC2)c2c1cnn1cc(-c3cnn4c3OCC4)c(-c3ccc4c(c3)OCO4)c21] |
| I-114 | 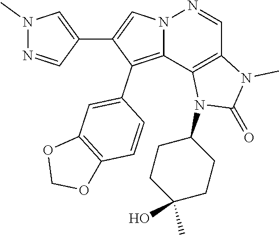 |
| I-114′ | 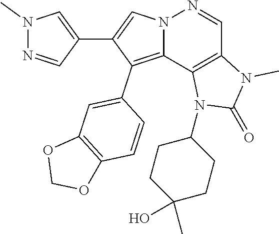 |
| I-115 | 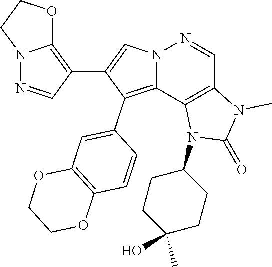 [SMILES: Cn1c(=O)n([C@H]2CC[C@@](C)(O)CC2)c2c1cnn1cc(-c3cnn4c3OCC4)c(-c3ccc4c(c3)OCCO4)c21] |
| I-115′ | 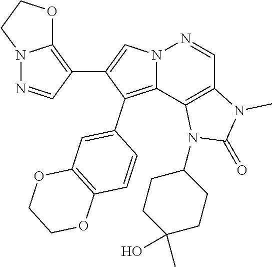 [SMILES: Cn1c(=O)n(C2CCC(C)(O)CC2)c2c1cnn1cc(-c3cnn4c3OCC4)c(-c3ccc4c(c3)OCCO4)c21] |
| I-116 | 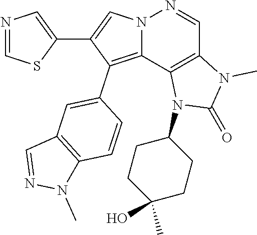 [SMILES: Cn1ncc2cc(-c3c(-c4cncs4)cn4ncc5c(c34)n([C@H]3CC[C@@](C)(O)CC3)c(=O)n5C)ccc21] |
| I-116′ | 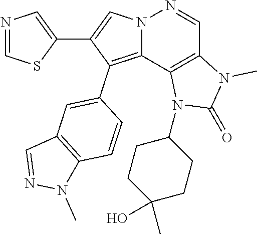 [SMILES: Cn1ncc2cc(-c3c(-c4cncs4)cn4ncc5c(c34)n(C3CCC(C)(O)CC3)c(=O)n5C)ccc21] |
| I-117 | 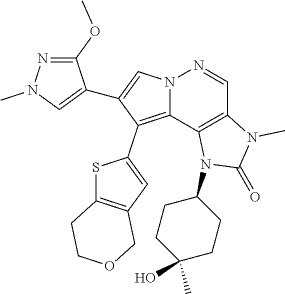 |
| I-117′ | 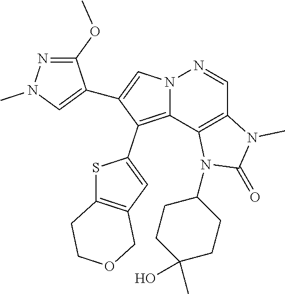 |
| I-118 | 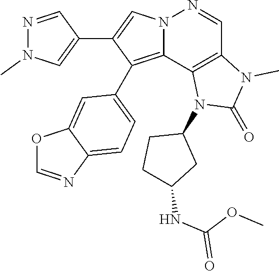 |
| I-118′ | 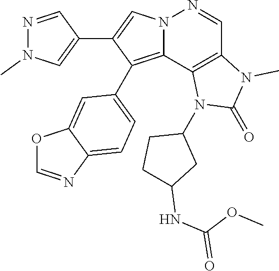 |
| I-119 | 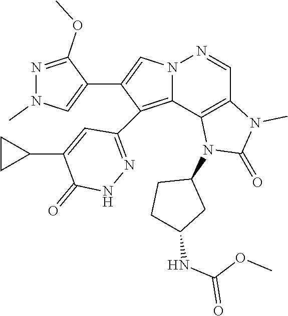 [SMILES: COC(=O)N[C@@H]1CC[C@@H](n2c(=O)n(C)c3cnn4cc(-c5cn(C)nc5OC)c(-c5cc(C6CC6)c(=O)[nH]n5)c4c32)C1] |
| I-119′ | 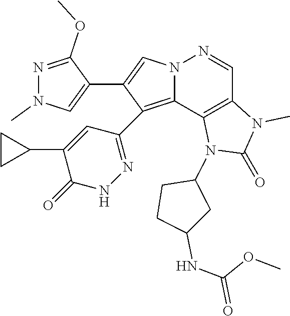 [SMILES: COC(=O)NC1CCC(n2c(=O)n(C)c3cnn4cc(-c5cn(C)nc5OC)c(-c5cc(C6CC6)c(=O)[nH]n5)c4c32)C1] |
| I-120 | 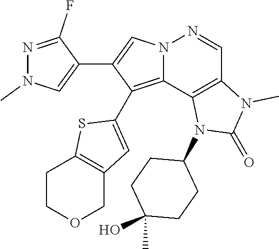 |
| I-120′ | 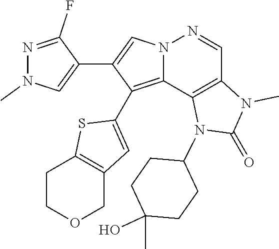 |
| I-121 | 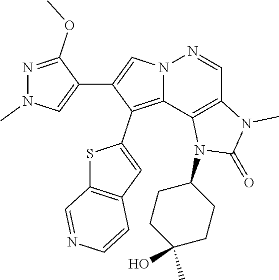 |
| I-121′ | 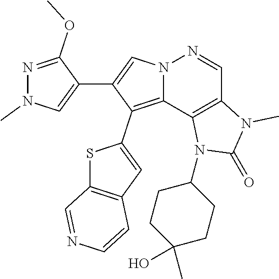 |
| I-122 | 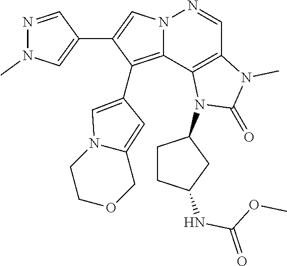 |
| I-122′ | 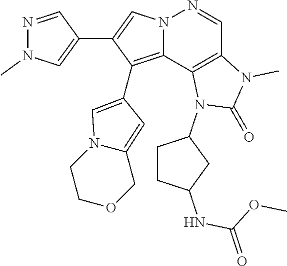 |
| I-123 | 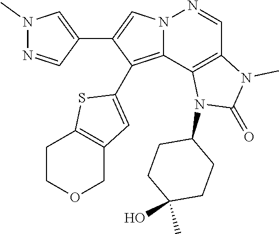 |
| I-123′ | 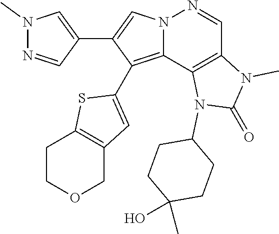 |
| I-124 | 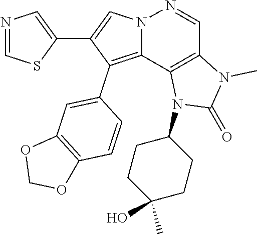 [SMILES: Cn1c(=O)n([C@H]2CC[C@@](C)(O)CC2)c2c1cnn1cc(-c3cncs3)c(-c3ccc4c(c3)OCO4)c21] |
| I-124′ | 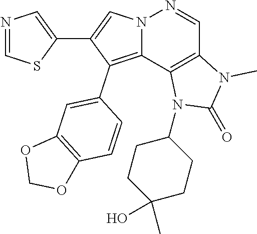 [SMILES: Cn1c(=O)n(C2CCC(C)(O)CC2)c2c1cnn1cc(-c3cncs3)c(-c3ccc4c(c3)OCO4)c21] |
| I-125 | 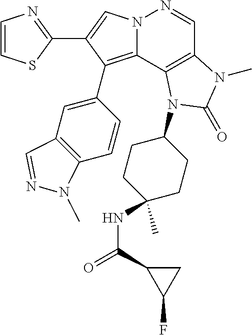 [SMILES: Cn1ncc2cc(-c3c(-c4nccs4)cn4ncc5c(c34)n([C@H]3CC[C@@](C)(NC(=O)[C@H]4C[C@H]4F)CC3)c(=O)n5C)ccc21] |
| I-125′ | 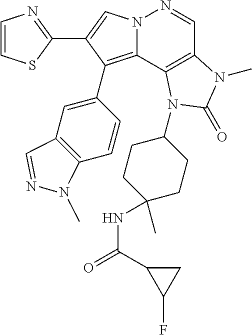 [SMILES: Cn1ncc2cc(-c3c(-c4nccs4)cn4ncc5c(c34)n(C3CCC(C)(NC(=O)C4CC4F)CC3)c(=O)n5C)ccc21] |
| I-126 | 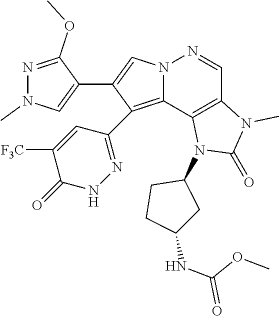 |
| I-126′ | 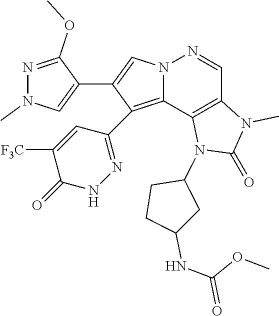 |
| I-127 | 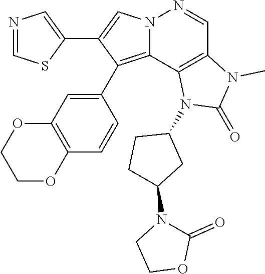 [SMILES: Cn1c(=O)n([C@H]2CC[C@H](N3CCOC3=O)C2)c2c1cnn1cc(-c3cncs3)c(-c3ccc4c(c3)OCCO4)c21] |
| I-127′ | 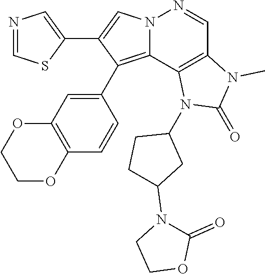 [SMILES: Cn1c(=O)n(C2CCC(N3CCOC3=O)C2)c2c1cnn1cc(-c3cncs3)c(-c3ccc4c(c3)OCCO4)c21] |
| I-128 | 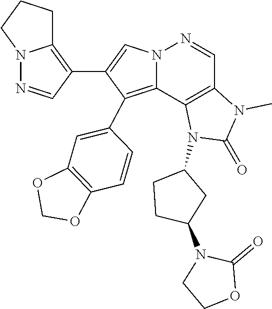 [SMILES: Cn1c(=O)n([C@H]2CC[C@H](N3CCOC3=O)C2)c2c1cnn1cc(-c3cnn4c3CCC4)c(-c3ccc4c(c3)OCO4)c21] |
| I-128′ | 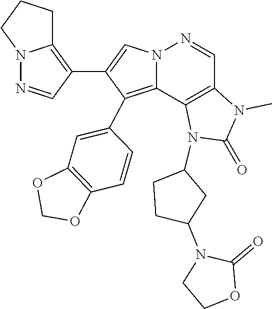 [SMILES: Cn1c(=O)n(C2CCC(N3CCOC3=O)C2)c2c1cnn1cc(-c3cnn4c3CCC4)c(-c3ccc4c(c3)OCO4)c21] |
| I-129 | 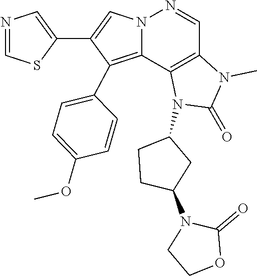 [SMILES: COc1ccc(-c2c(-c3cncs3)cn3ncc4c(c23)n([C@H]2CC[C@H](N3CCOC3=O)C2)c(=O)n4C)cc1] |
| I-129′ | 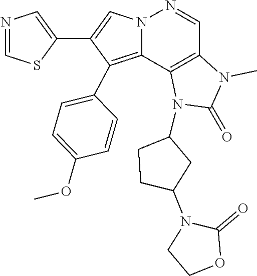 [SMILES: COc1ccc(-c2c(-c3cncs3)cn3ncc4c(c23)n(C2CCC(N3CCOC3=O)C2)c(=O)n4C)cc1] |
| I-130 | 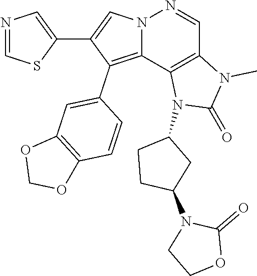 [SMILES: Cn1c(=O)n([C@H]2CC[C@H](N3CCOC3=O)C2)c2c1cnn1cc(-c3cncs3)c(-c3ccc4c(c3)OCO4)c21] |
| I-130′ | 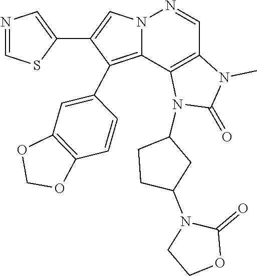 [SMILES: Cn1c(=O)n(C2CCC(N3CCOC3=O)C2)c2c1cnn1cc(-c3cncs3)c(-c3ccc4c(c3)OCO4)c21] |
| I-131 | 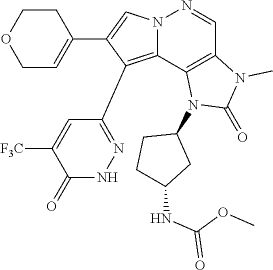 [SMILES: COC(=O)N[C@@H]1CC[C@@H](n2c(=O)n(C)c3cnn4cc(C5=CCOCC5)c(-c5cc(C(F)(F)F)c(=O)[nH]n5)c4c32)C1] |
| I-131′ | 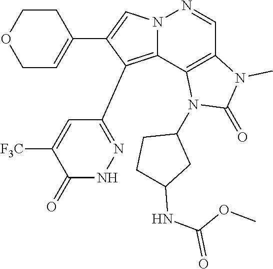 [SMILES: COC(=O)NC1CCC(n2c(=O)n(C)c3cnn4cc(C5=CCOCC5)c(-c5cc(C(F)(F)F)c(=O)[nH]n5)c4c32)C1] |
| I-132 | 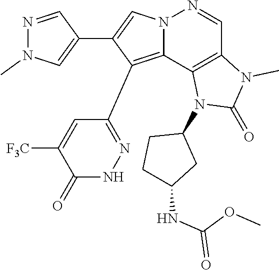 |
| I-132′ | 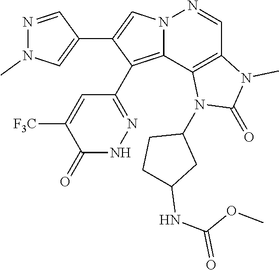 |
| I-133 | 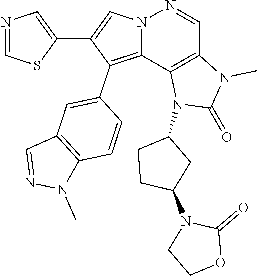 [SMILES: Cn1ncc2cc(-c3c(-c4cncs4)cn4ncc5c(c34)n([C@H]3CC[C@H](N4CCOC4=O)C3)c(=O)n5C)ccc21] |
| I-133′ | 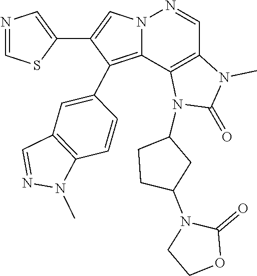 [SMILES: Cn1ncc2cc(-c3c(-c4cncs4)cn4ncc5c(c34)n(C3CCC(N4CCOC4=O)C3)c(=O)n5C)ccc21] |
| I-134 | 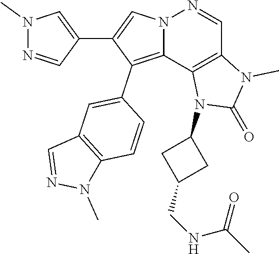 |
| I-134′ | 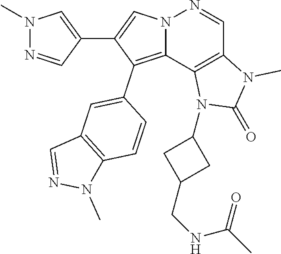 |
| I-135 | 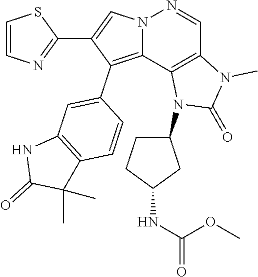 [SMILES: COC(=O)N[C@@H]1CC[C@@H](n2c(=O)n(C)c3cnn4cc(-c5nccs5)c(-c5ccc6c(c5)NC(=O)C6(C)C)c4c32)C1] |
| I-135′ | 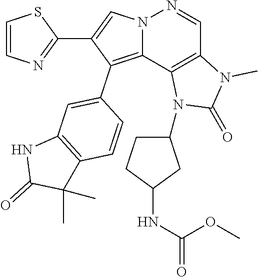 [SMILES: COC(=O)NC1CCC(n2c(=O)n(C)c3cnn4cc(-c5nccs5)c(-c5ccc6c(c5)NC(=O)C6(C)C)c4c32)C1] |
| I-136 | 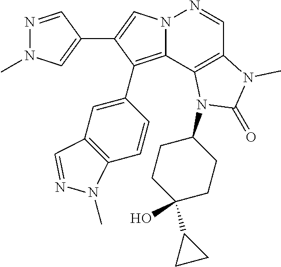 |
| I-136′ | 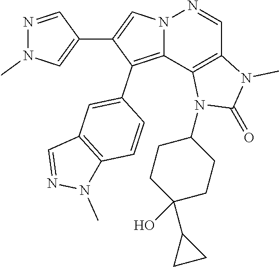 |
| I-137 | 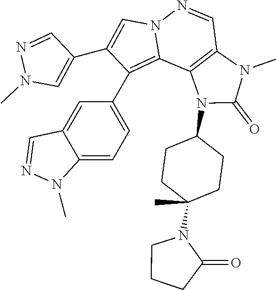 |
| I-137′ | 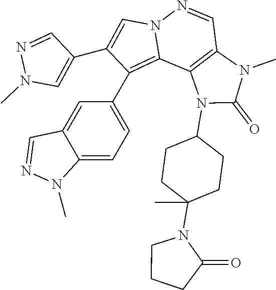 |
| I-138 | 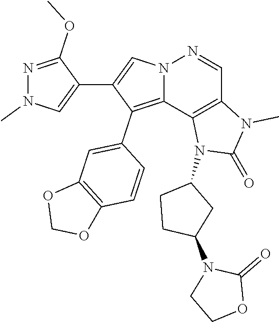 |
| I-138′ | 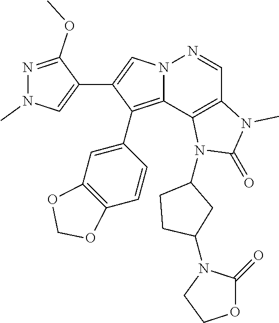 |
| I-139 | 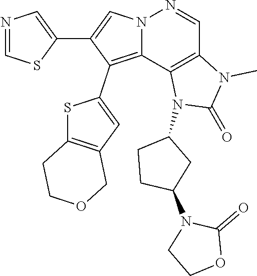 [SMILES: Cn1c(=O)n([C@H]2CC[C@H](N3CCOC3=O)C2)c2c1cnn1cc(-c3cncs3)c(-c3cc4c(s3)CCOC4)c21] |
| I-139′ | 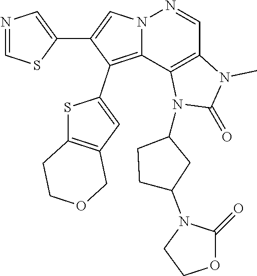 [SMILES: Cn1c(=O)n(C2CCC(N3CCOC3=O)C2)c2c1cnn1cc(-c3cncs3)c(-c3cc4c(s3)CCOC4)c21] |
| I-140 | 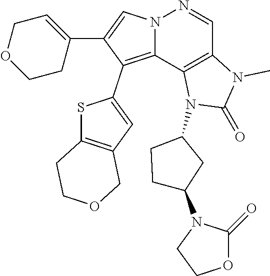 [SMILES: Cn1c(=O)n([C@H]2CC[C@H](N3CCOC3=O)C2)c2c1cnn1cc(C3=CCOCC3)c(-c3cc4c(s3)CCOC4)c21] |
| I-140′ | 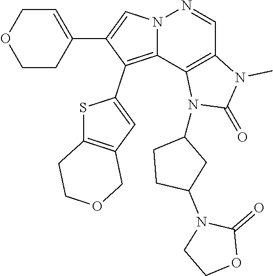 [SMILES: Cn1c(=O)n(C2CCC(N3CCOC3=O)C2)c2c1cnn1cc(C3=CCOCC3)c(-c3cc4c(s3)CCOC4)c21] |
| I-141 | 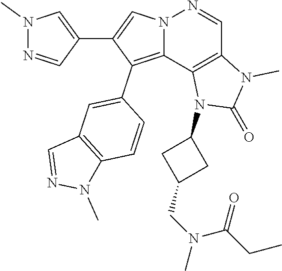 [SMILES: CCC(=O)N(C)C[C@H]1C[C@H](n2c(=O)n(C)c3cnn4cc(-c5cnn(C)c5)c(-c5ccc6c(cnn6C)c5)c4c32)C1] |
| I-141′ | 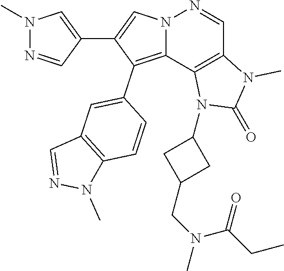 [SMILES: CCC(=O)N(C)CC1CC(n2c(=O)n(C)c3cnn4cc(-c5cnn(C)c5)c(-c5ccc6c(cnn6C)c5)c4c32)C1] |
| I-142 | 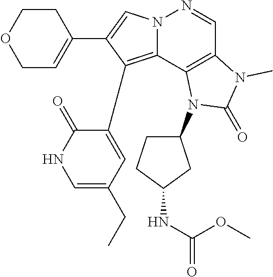 [SMILES: CCc1c[nH]c(=O)c(-c2c(C3=CCOCC3)cn3ncc4c(c23)n([C@@H]2CC[C@@H](NC(=O)OC)C2)c(=O)n4C)c1] |
| I-142′ | 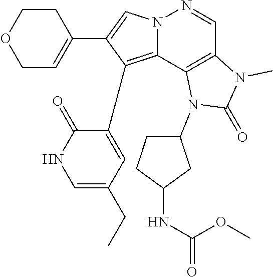 [SMILES: CCc1c[nH]c(=O)c(-c2c(C3=CCOCC3)cn3ncc4c(c23)n(C2CCC(NC(=O)OC)C2)c(=O)n4C)c1] |
| I-143 | 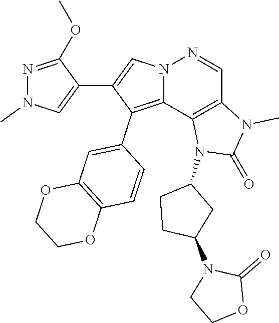 |
| I-143′ | 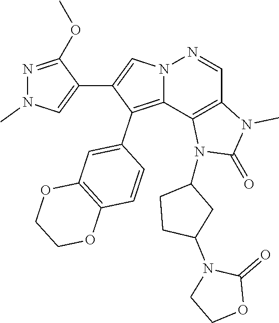 |
| I-144 | 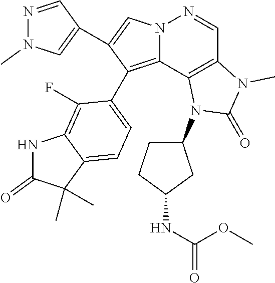 |
| I-144′ | 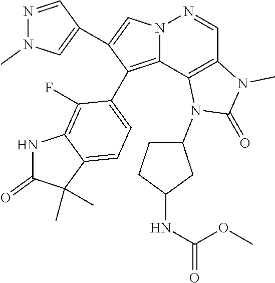 |
| I-145 | 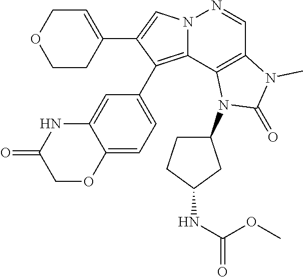 [SMILES: COC(=O)N[C@@H]1CC[C@@H](n2c(=O)n(C)c3cnn4cc(C5=CCOCC5)c(-c5ccc6c(c5)NC(=O)CO6)c4c32)C1] |
| I-145′ | 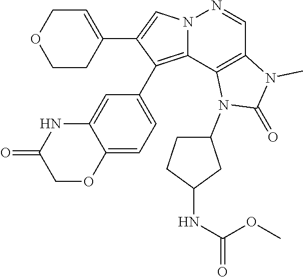 [SMILES: COC(=O)NC1CCC(n2c(=O)n(C)c3cnn4cc(C5=CCOCC5)c(-c5ccc6c(c5)NC(=O)CO6)c4c32)C1] |
| I-146 | 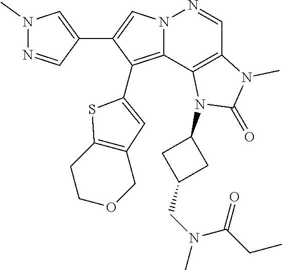 [SMILES: CCC(=O)N(C)C[C@H]1C[C@H](n2c(=O)n(C)c3cnn4cc(-c5cnn(C)c5)c(-c5cc6c(s5)CCOC6)c4c32)C1] |
| I-146′ | 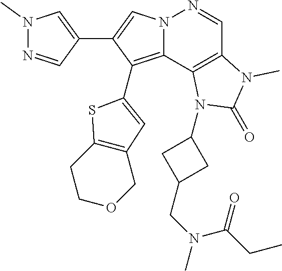 [SMILES: CCC(=O)N(C)CC1CC(n2c(=O)n(C)c3cnn4cc(-c5cnn(C)c5)c(-c5cc6c(s5)CCOC6)c4c32)C1] |
| I-147 | 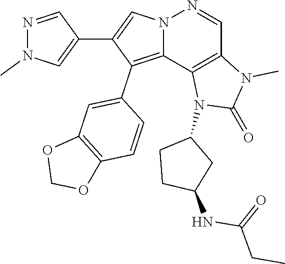 [SMILES: CCC(=O)N[C@H]1CC[C@H](n2c(=O)n(C)c3cnn4cc(-c5cnn(C)c5)c(-c5ccc6c(c5)OCO6)c4c32)C1] |
| I-147′ | 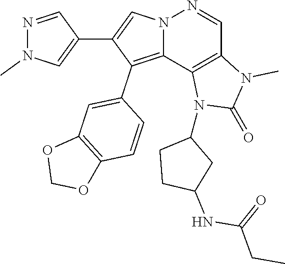 [SMILES: CCC(=O)NC1CCC(n2c(=O)n(C)c3cnn4cc(-c5cnn(C)c5)c(-c5ccc6c(c5)OCO6)c4c32)C1] |
| I-148 | 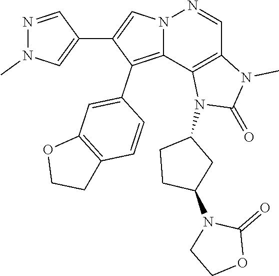 |
| I-148′ | 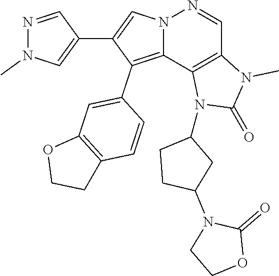 |
| I-149 | 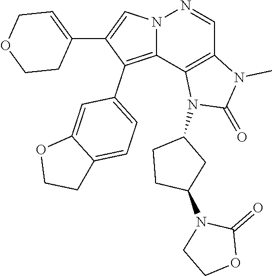 [SMILES: Cn1c(=O)n([C@H]2CC[C@H](N3CCOC3=O)C2)c2c1cnn1cc(C3=CCOCC3)c(-c3ccc4c(c3)OCC4)c21] |
| I-149′ | 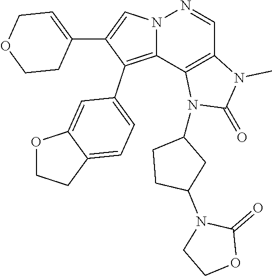 [SMILES: Cn1c(=O)n(C2CCC(N3CCOC3=O)C2)c2c1cnn1cc(C3=CCOCC3)c(-c3ccc4c(c3)OCC4)c21] |
| I-150 | 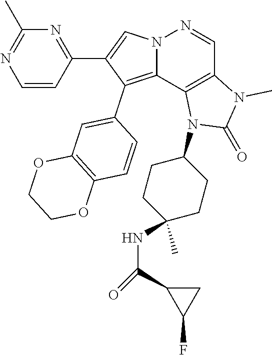 [SMILES: Cc1nccc(-c2cn3ncc4c(c3c2-c2ccc3c(c2)OCCO3)n([C@H]2CC[C@@](C)(NC(=O)[C@H]3C[C@H]3F)CC2)c(=O)n4C)n1] |
| I-150′ | 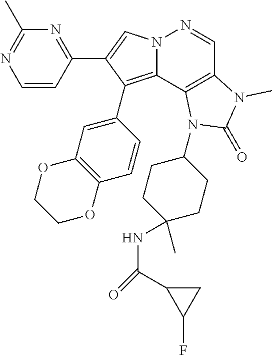 [SMILES: Cc1nccc(-c2cn3ncc4c(c3c2-c2ccc3c(c2)OCCO3)n(C2CCC(C)(NC(=O)C3CC3F)CC2)c(=O)n4C)n1] |
| I-151 | 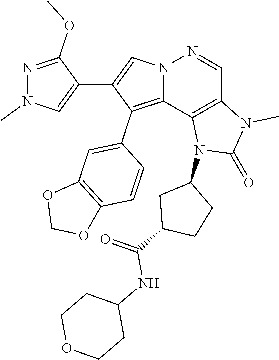 |
| I-151′ | 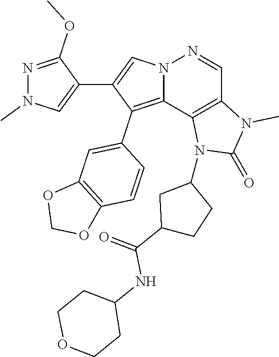 |
| I-152 | 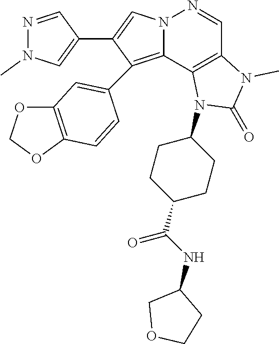 |
| I-152′ | 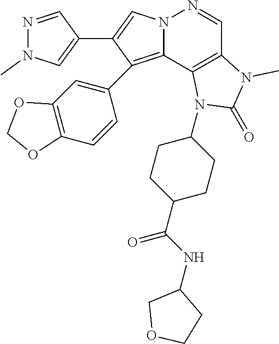 |
| I-153 | 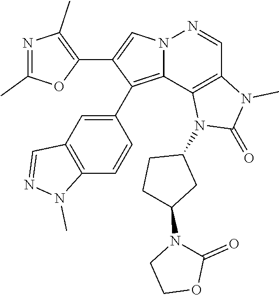 |
| I-153′ | 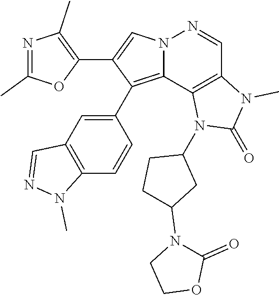 |
| I-154 | 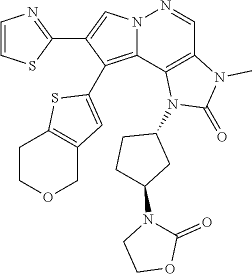 [SMILES: Cn1c(=O)n([C@H]2CC[C@H](N3CCOC3=O)C2)c2c1cnn1cc(-c3nccs3)c(-c3cc4c(s3)CCOC4)c21] |
| I-154′ | 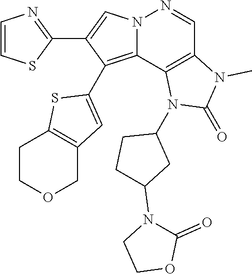 [SMILES: Cn1c(=O)n(C2CCC(N3CCOC3=O)C2)c2c1cnn1cc(-c3nccs3)c(-c3cc4c(s3)CCOC4)c21] |
| I-155 | 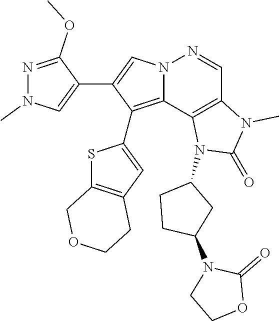 |
| I-155′ | 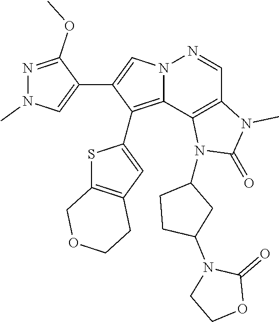 |
| I-156 | 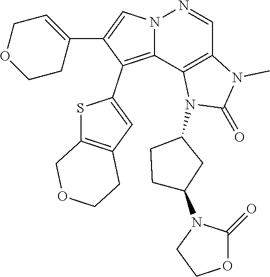 [SMILES: Cn1c(=O)n([C@H]2CC[C@H](N3CCOC3=O)C2)c2c1cnn1cc(C3=CCOCC3)c(-c3cc4c(s3)COCC4)c21] |
| I-156′ | 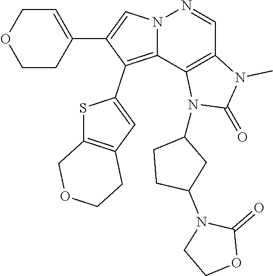 [SMILES: Cn1c(=O)n(C2CCC(N3CCOC3=O)C2)c2c1cnn1cc(C3=CCOCC3)c(-c3cc4c(s3)COCC4)c21] |
| I-157 | 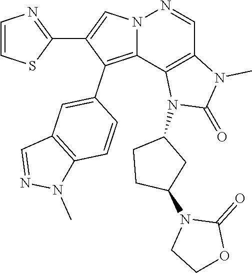 [SMILES: Cn1ncc2cc(-c3c(-c4nccs4)cn4ncc5c(c34)n([C@H]3CC[C@H](N4CCOC4=O)C3)c(=O)n5C)ccc21] |
| I-157′ | 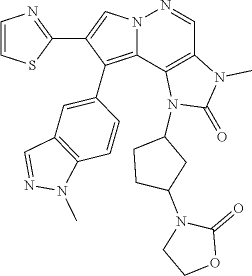 [SMILES: Cn1ncc2cc(-c3c(-c4nccs4)cn4ncc5c(c34)n(C3CCC(N4CCOC4=O)C3)c(=O)n5C)ccc21] |
| I-158 | 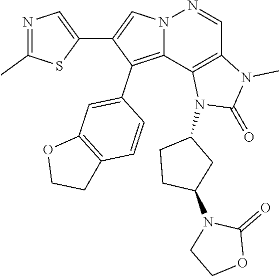 |
| I-158′ | 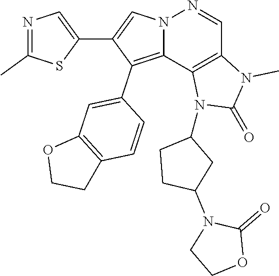 |
| I-159 | 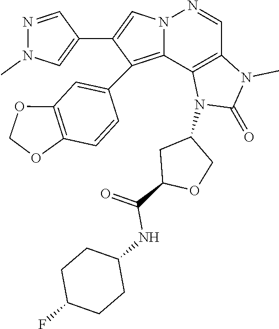 |
| I-159′ | 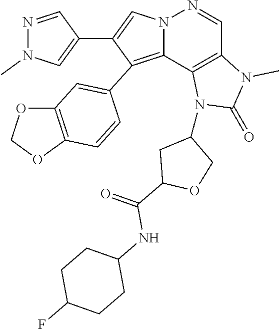 |
| I-160 | 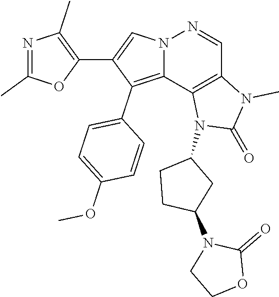 |
| I-160′ | 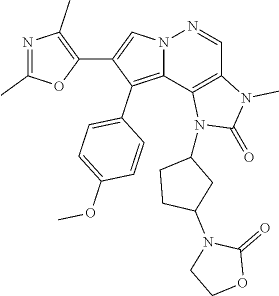 |
| I-161 | 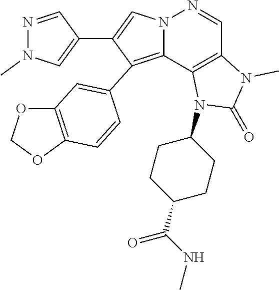 |
| I-161′ | 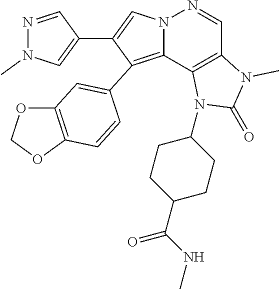 |
| I-162 | 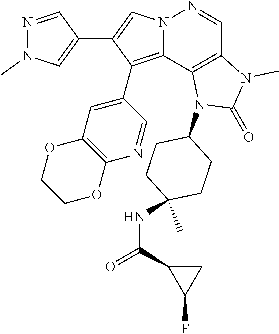 |
| I-162′ | 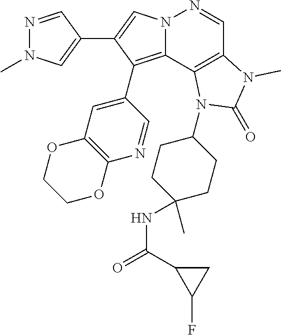 |
| I-163 | 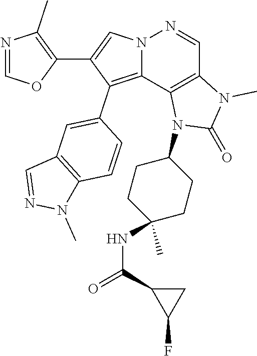 [SMILES: Cc1ncoc1-c1cn2ncc3c(c2c1-c1ccc2c(cnn2C)c1)n([C@H]1CC[C@@](C)(NC(=O)[C@H]2C[C@H]2F)CC1)c(=O)n3C] |
| I-163′ | 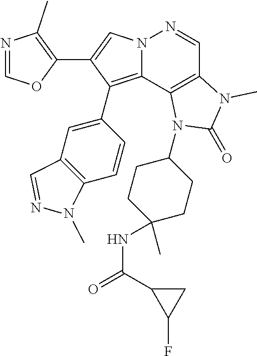 [SMILES: Cc1ncoc1-c1cn2ncc3c(c2c1-c1ccc2c(cnn2C)c1)n(C1CCC(C)(NC(=O)C2CC2F)CC1)c(=O)n3C] |
| I-164 | 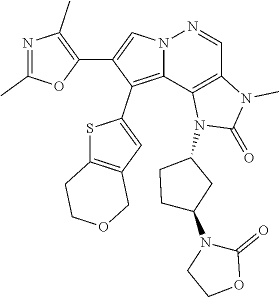 |
| I-164′ | 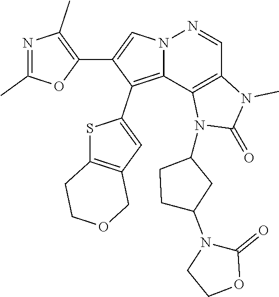 |
| I-165 | 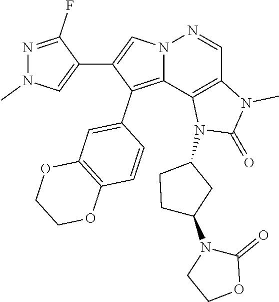 |
| I-165′ | 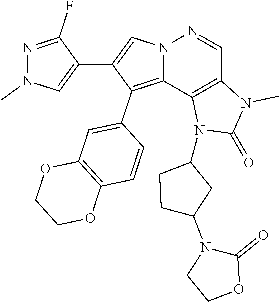 |
| I-166-1 | 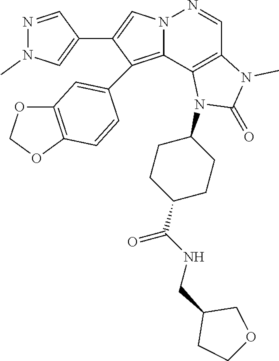 |
| I-166-2 | 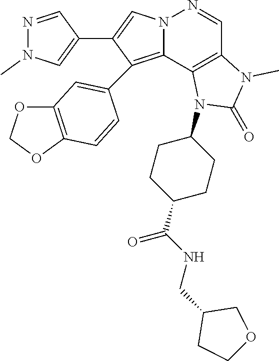 |
| I-166′ | 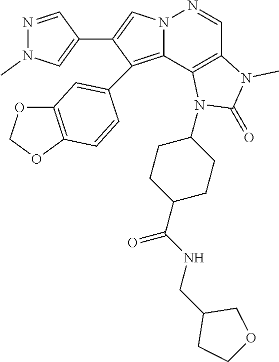 |
| I-167 | 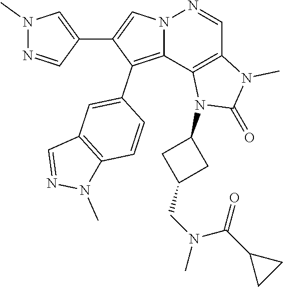 [SMILES: CN(C[C@H]1C[C@H](n2c(=O)n(C)c3cnn4cc(-c5cnn(C)c5)c(-c5ccc6c(cnn6C)c5)c4c32)C1)C(=O)C1CC1] |
| I-167′ | 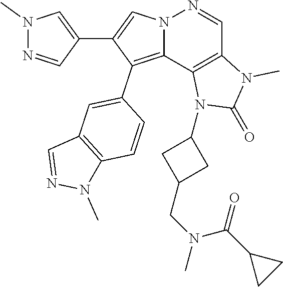 [SMILES: CN(CC1CC(n2c(=O)n(C)c3cnn4cc(-c5cnn(C)c5)c(-c5ccc6c(cnn6C)c5)c4c32)C1)C(=O)C1CC1] |
| I-168 | 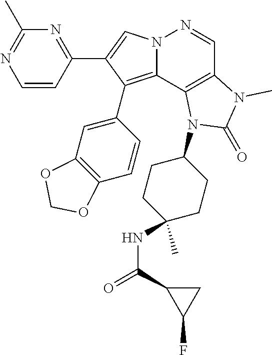 [SMILES: Cc1nccc(-c2cn3ncc4c(c3c2-c2ccc3c(c2)OCO3)n([C@H]2CC[C@@](C)(NC(=O)[C@H]3C[C@H]3F)CC2)c(=O)n4C)n1] |
| I-168′ | 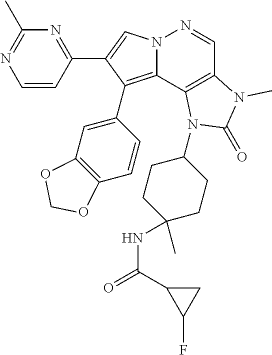 [SMILES: Cc1nccc(-c2cn3ncc4c(c3c2-c2ccc3c(c2)OCO3)n(C2CCC(C)(NC(=O)C3CC3F)CC2)c(=O)n4C)n1] |
| I-169 | 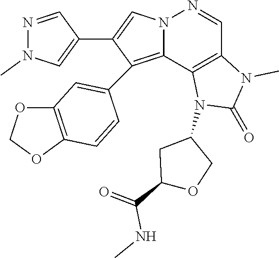 |
| I-169′ | 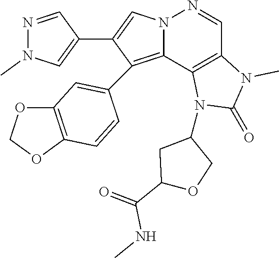 |
| I-170 | 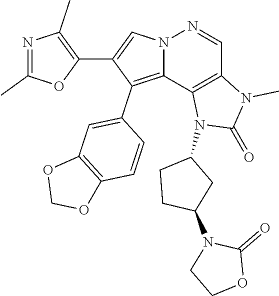 |
| I-170′ | 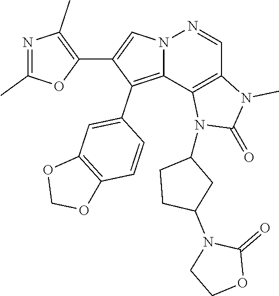 |
| I-171 | 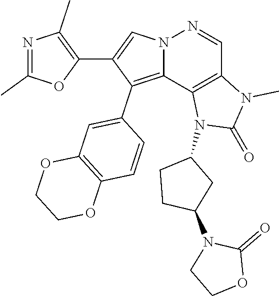 |
| I-171′ | 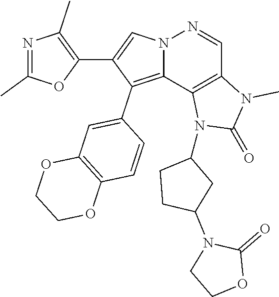 |
| I-172 | 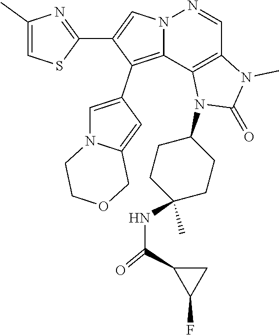 |
| I-172′ | 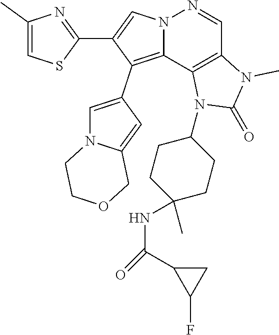 |
| I-173 | 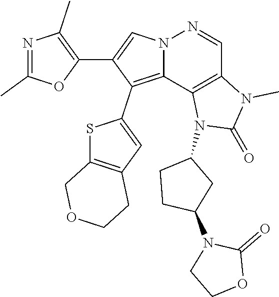 |
| I-173′ | 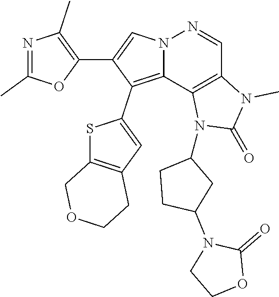 |
| I-174 | 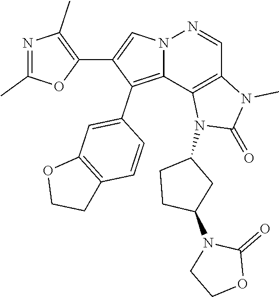 |
| I-174′ | 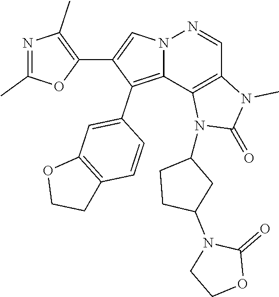 |
| I-175 | 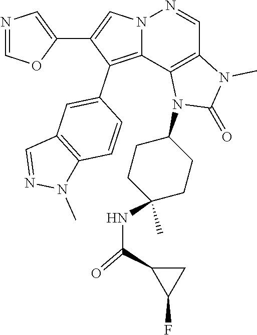 [SMILES: Cn1ncc2cc(-c3c(-c4cnco4)cn4ncc5c(c34)n([C@H]3CC[C@@](C)(NC(=O)[C@H]4C[C@H]4F)CC3)c(=O)n5C)ccc21] |
| I-175′ | 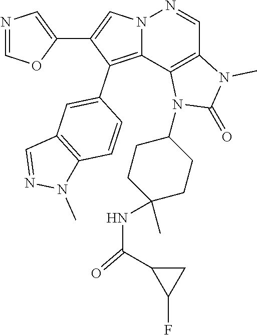 [SMILES: Cn1ncc2cc(-c3c(-c4cnco4)cn4ncc5c(c34)n(C3CCC(C)(NC(=O)C4CC4F)CC3)c(=O)n5C)ccc21] |
| I-176 | 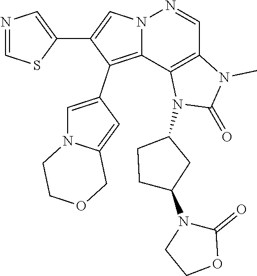 [SMILES: Cn1c(=O)n([C@H]2CC[C@H](N3CCOC3=O)C2)c2c1cnn1cc(-c3cncs3)c(-c3cc4n(c3)CCOC4)c21] |
| I-176′ | 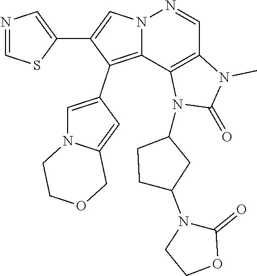 [SMILES: Cn1c(=O)n(C2CCC(N3CCOC3=O)C2)c2c1cnn1cc(-c3cncs3)c(-c3cc4n(c3)CCOC4)c21] |
| I-177 | 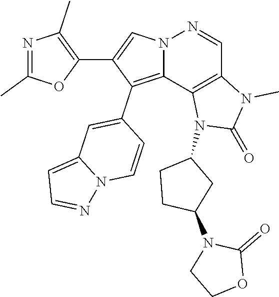 |
| I-177′ | 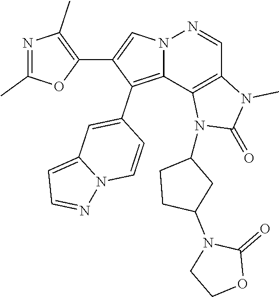 |
| I-178 | 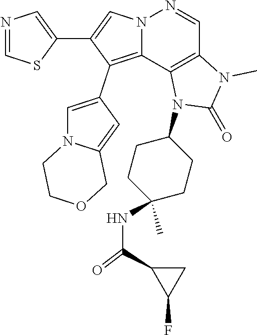 [SMILES: Cn1c(=O)n([C@H]2CC[C@@](C)(NC(=O)[C@H]3C[C@H]3F)CC2)c2c1cnn1cc(-c3cncs3)c(-c3cc4n(c3)CCOC4)c21] |
| I-178′ | 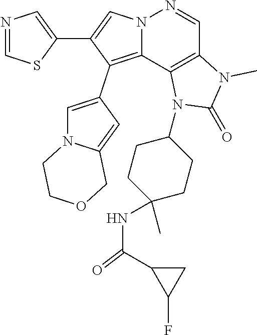 [SMILES: Cn1c(=O)n(C2CCC(C)(NC(=O)C3CC3F)CC2)c2c1cnn1cc(-c3cncs3)c(-c3cc4n(c3)CCOC4)c21] |
| I-179-1 | 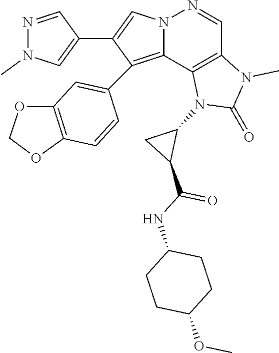 |
| I-179-2 | 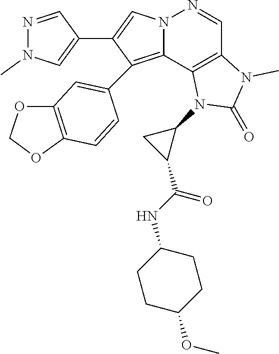 |
| I-179′ | 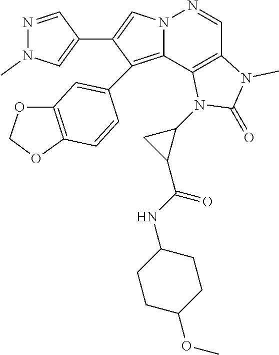 |
| I-180 | 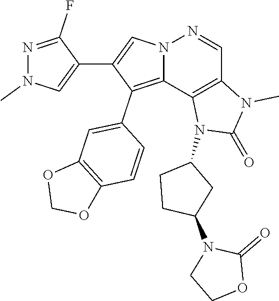 |
| I-180′ | 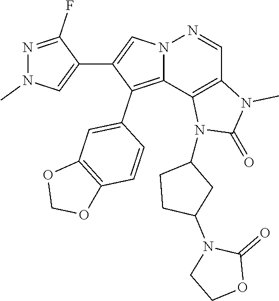 |
| I-181 | 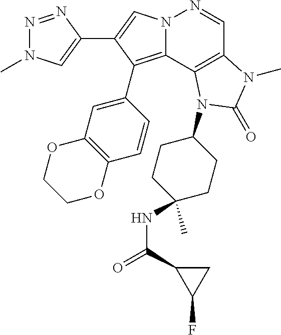 |
| I-181′ | 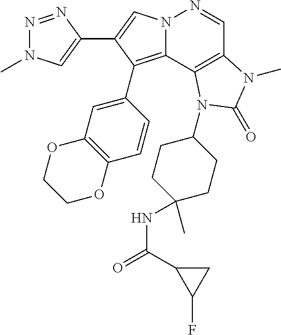 |
| I-182 | 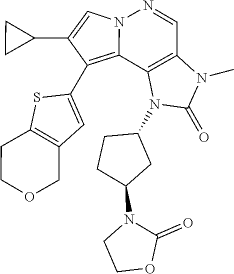 [SMILES: Cn1c(=O)n([C@H]2CC[C@H](N3CCOC3=O)C2)c2c1cnn1cc(C3CC3)c(-c3cc4c(s3)CCOC4)c21] |
| I-182′ | 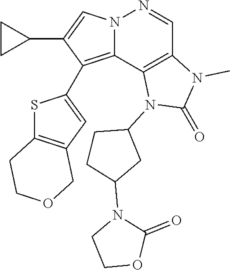 [SMILES: Cn1c(=O)n(C2CCC(N3CCOC3=O)C2)c2c1cnn1cc(C3CC3)c(-c3cc4c(s3)CCOC4)c21] |
| I-183 | 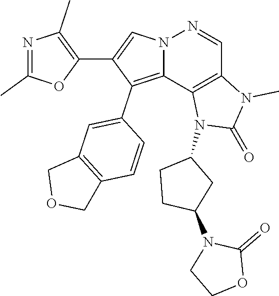 |
| I-183′ | 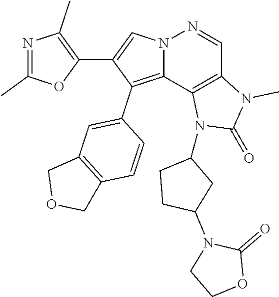 |
| I-184 | 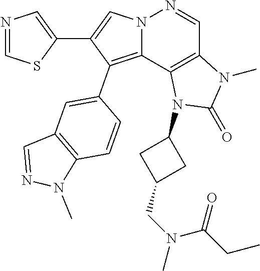 [SMILES: CCC(=O)N(C)C[C@H]1C[C@H](n2c(=O)n(C)c3cnn4cc(-c5cncs5)c(-c5ccc6c(cnn6C)c5)c4c32)C1] |
| I-184′ | 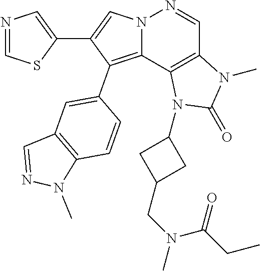 [SMILES: CCC(=O)N(C)CC1CC(n2c(=O)n(C)c3cnn4cc(-c5cncs5)c(-c5ccc6c(cnn6C)c5)c4c32)C1] |
| I-185 | 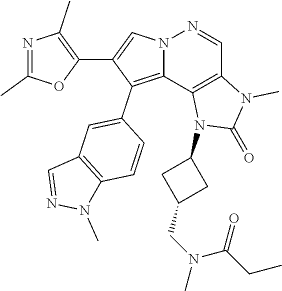 [SMILES: CCC(=O)N(C)C[C@H]1C[C@H](n2c(=O)n(C)c3cnn4cc(-c5oc(C)nc5C)c(-c5ccc6c(cnn6C)c5)c4c32)C1] |
| I-185′ | 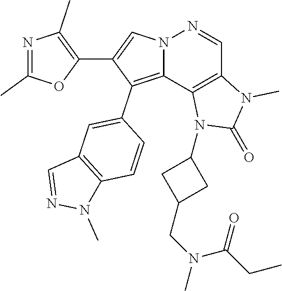 [SMILES: CCC(=O)N(C)CC1CC(n2c(=O)n(C)c3cnn4cc(-c5oc(C)nc5C)c(-c5ccc6c(cnn6C)c5)c4c32)C1] |
| I-186 | 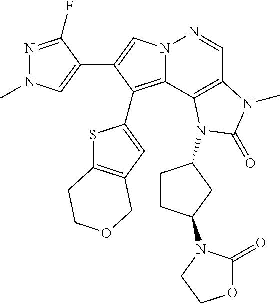 |
| I-186′ | 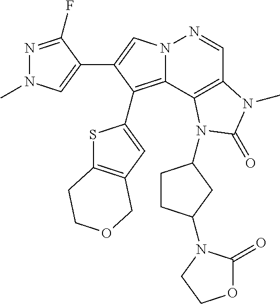 |
| I-187 | 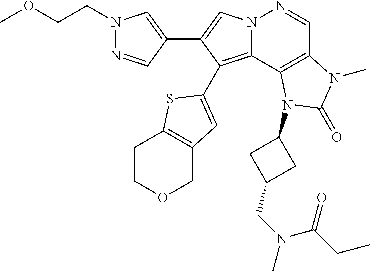 [SMILES: CCC(=O)N(C)C[C@H]1C[C@H](n2c(=O)n(C)c3cnn4cc(-c5cnn(CCOC)c5)c(-c5cc6c(s5)CCOC6)c4c32)C1] |
| I-187′ | 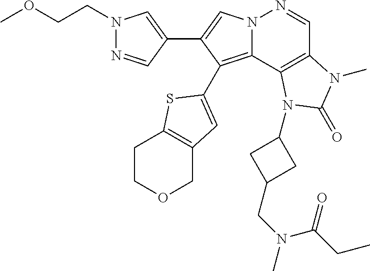 [SMILES: CCC(=O)N(C)CC1CC(n2c(=O)n(C)c3cnn4cc(-c5cnn(CCOC)c5)c(-c5cc6c(s5)CCOC6)c4c32)C1] |
| I-188 | 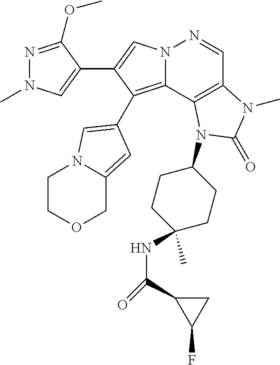 |
| I-188′ | 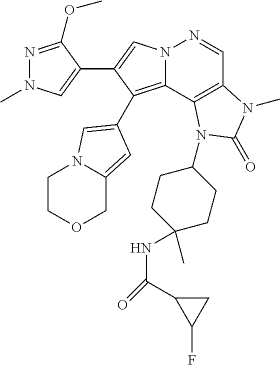 |
| I-189 | 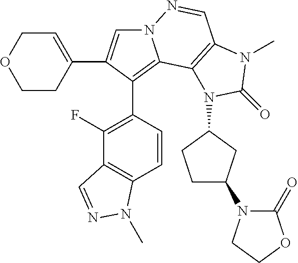 [SMILES: Cn1ncc2c(F)c(-c3c(C4=CCOCC4)cn4ncc5c(c34)n([C@H]3CC[C@H](N4CCOC4=O)C3)c(=O)n5C)ccc21] |
| I-189′ | 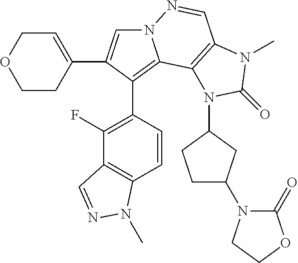 [SMILES: Cn1ncc2c(F)c(-c3c(C4=CCOCC4)cn4ncc5c(c34)n(C3CCC(N4CCOC4=O)C3)c(=O)n5C)ccc21] |
| I-190 | 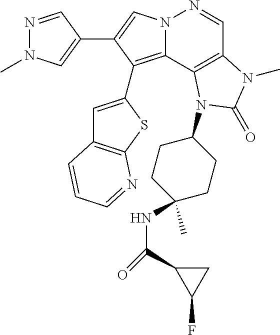 |
| I-190′ | 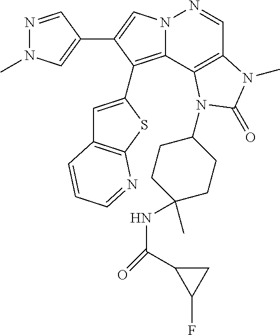 |
| I-191 | 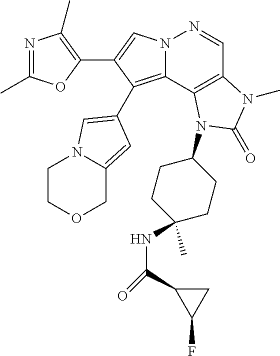 |
| I-191′ | 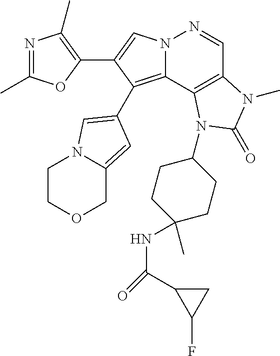 |
| I-192 | 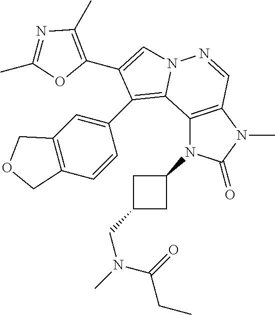 |
| I-192′ | 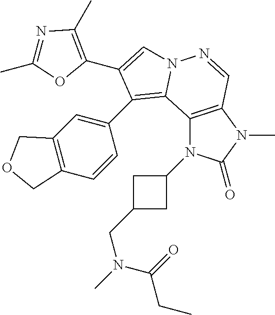 |
| I-193 | 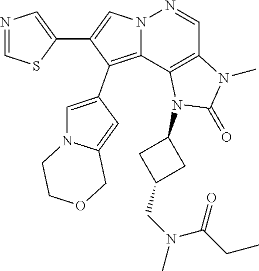 [SMILES: CCC(=O)N(C)C[C@H]1C[C@H](n2c(=O)n(C)c3cnn4cc(-c5cncs5)c(-c5cc6n(c5)CCOC6)c4c32)C1] |
| I-193′ | 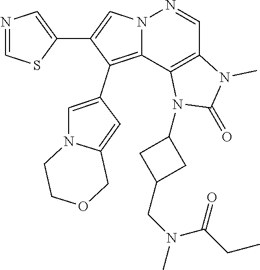 [SMILES: CCC(=O)N(C)CC1CC(n2c(=O)n(C)c3cnn4cc(-c5cncs5)c(-c5cc6n(c5)CCOC6)c4c32)C1] |
| I-194 | 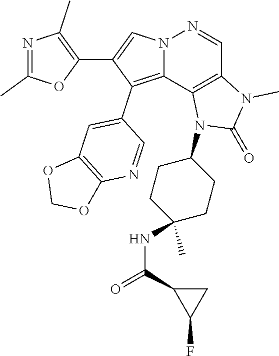 |
| I-194′ | 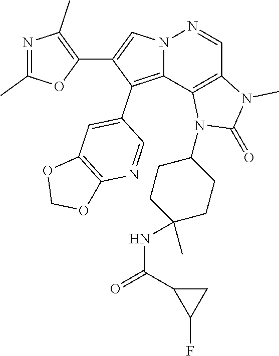 |
| I-195 | 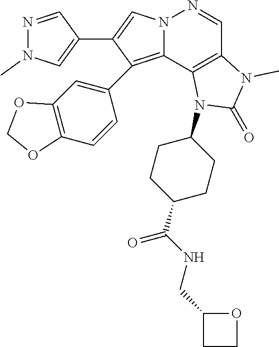 |
| I-195′ | 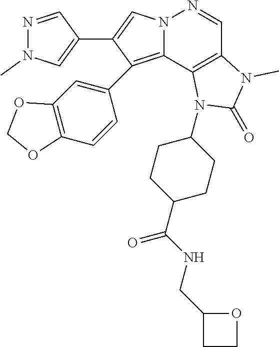 |
| I-196 | 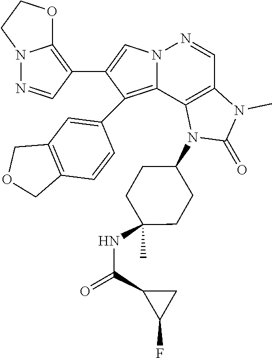 [SMILES: Cn1c(=O)n([C@H]2CC[C@@](C)(NC(=O)[C@H]3C[C@H]3F)CC2)c2c1cnn1cc(-c3cnn4c3OCC4)c(-c3ccc4c(c3)COC4)c21] |
| I-196′ | 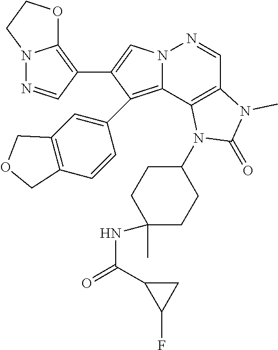 [SMILES: Cn1c(=O)n(C2CCC(C)(NC(=O)C3CC3F)CC2)c2c1cnn1cc(-c3cnn4c3OCC4)c(-c3ccc4c(c3)COC4)c21] |
| I-197 | 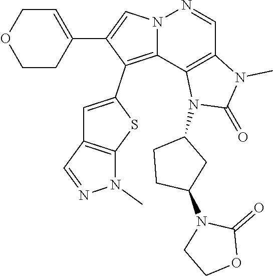 [SMILES: Cn1ncc2cc(-c3c(C4=CCOCC4)cn4ncc5c(c34)n([C@H]3CC[C@H](N4CCOC4=O)C3)c(=O)n5C)sc21] |
| I-197′ | 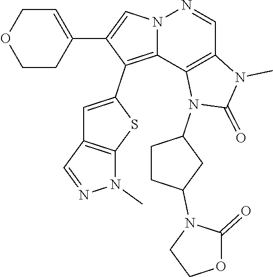 [SMILES: Cn1ncc2cc(-c3c(C4=CCOCC4)cn4ncc5c(c34)n(C3CCC(N4CCOC4=O)C3)c(=O)n5C)sc21] |
| I-198 | 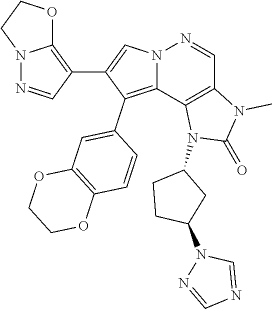 [SMILES: Cn1c(=O)n([C@H]2CC[C@H](n3cncn3)C2)c2c1cnn1cc(-c3cnn4c3OCC4)c(-c3ccc4c(c3)OCCO4)c21] |
| I-198′ | 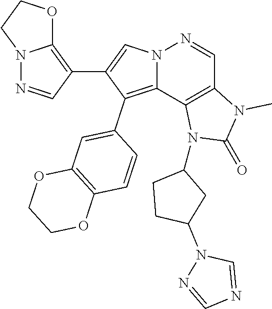 [SMILES: Cn1c(=O)n(C2CCC(n3cncn3)C2)c2c1cnn1cc(-c3cnn4c3OCC4)c(-c3ccc4c(c3)OCCO4)c21] |
| I-199 | 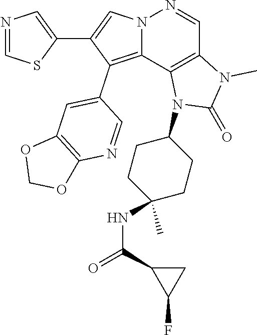 [SMILES: Cn1c(=O)n([C@H]2CC[C@@](C)(NC(=O)[C@H]3C[C@H]3F)CC2)c2c1cnn1cc(-c3cncs3)c(-c3cnc4c(c3)OCO4)c21] |
| I-199′ | 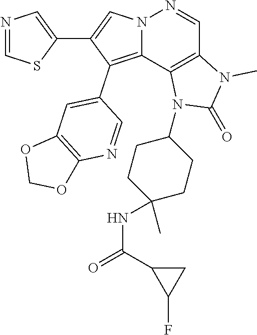 [SMILES: Cn1c(=O)n(C2CCC(C)(NC(=O)C3CC3F)CC2)c2c1cnn1cc(-c3cncs3)c(-c3cnc4c(c3)OCO4)c21] |
| I-200 | 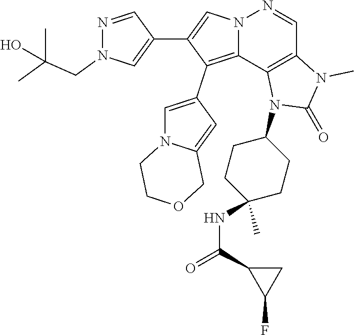 [SMILES: Cn1c(=O)n([C@H]2CC[C@@](C)(NC(=O)[C@H]3C[C@H]3F)CC2)c2c1cnn1cc(-c3cnn(CC(C)(C)O)c3)c(-c3cc4n(c3)CCOC4)c21] |
| I-200′ | 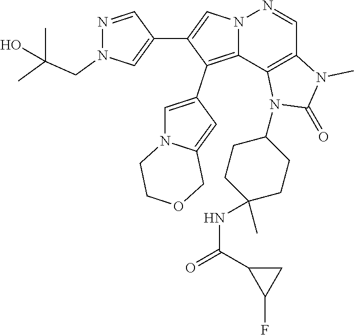 [SMILES: Cn1c(=O)n(C2CCC(C)(NC(=O)C3CC3F)CC2)c2c1cnn1cc(-c3cnn(CC(C)(C)O)c3)c(-c3cc4n(c3)CCOC4)c21] |
| I-201 | 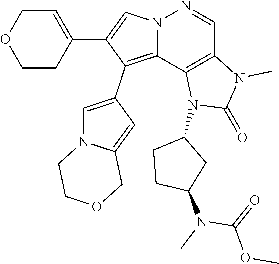 [SMILES: COC(=O)N(C)[C@H]1CC[C@H](n2c(=O)n(C)c3cnn4cc(C5=CCOCC5)c(-c5cc6n(c5)CCOC6)c4c32)C1] |
| I-201′ | 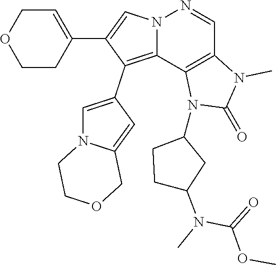 [SMILES: COC(=O)N(C)C1CCC(n2c(=O)n(C)c3cnn4cc(C5=CCOCC5)c(-c5cc6n(c5)CCOC6)c4c32)C1] |
| I-202 | 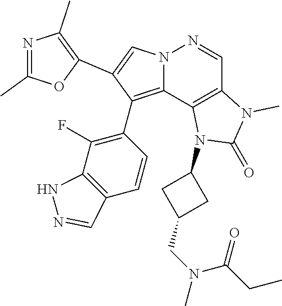 [SMILES: CCC(=O)N(C)C[C@H]1C[C@H](n2c(=O)n(C)c3cnn4cc(-c5oc(C)nc5C)c(-c5ccc6cn[nH]c6c5F)c4c32)C1] |
| I-202′ | 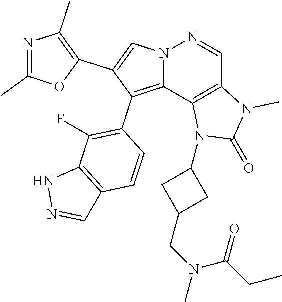 [SMILES: CCC(=O)N(C)CC1CC(n2c(=O)n(C)c3cnn4cc(-c5oc(C)nc5C)c(-c5ccc6cn[nH]c6c5F)c4c32)C1] |
| I-203 | 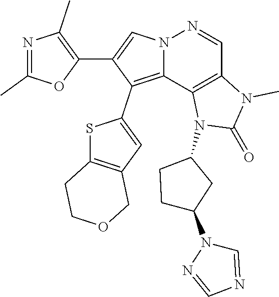 |
| I-203′ | 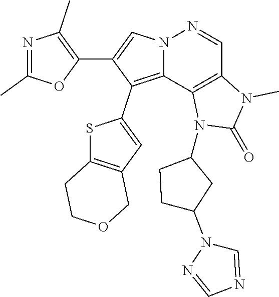 |
| I-204 | 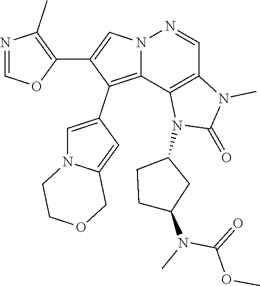 [SMILES: COC(=O)N(C)[C@H]1CC[C@H](n2c(=O)n(C)c3cnn4cc(-c5ocnc5C)c(-c5cc6n(c5)CCOC6)c4c32)C1] |
| I-204′ | 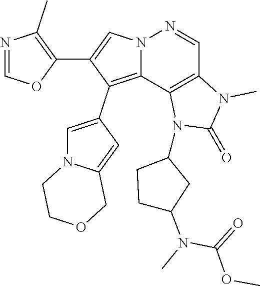 [SMILES: COC(=O)N(C)C1CCC(n2c(=O)n(C)c3cnn4cc(-c5ocnc5C)c(-c5cc6n(c5)CCOC6)c4c32)C1] |
| I-205 | 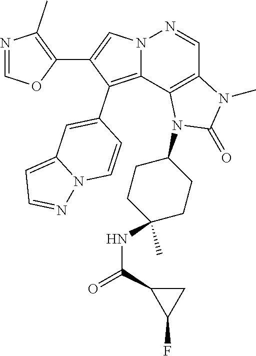 [SMILES: Cc1ncoc1-c1cn2ncc3c(c2c1-c1ccn2nccc2c1)n([C@H]1CC[C@@](C)(NC(=O)[C@H]2C[C@H]2F)CC1)c(=O)n3C] |
| I-205′ | 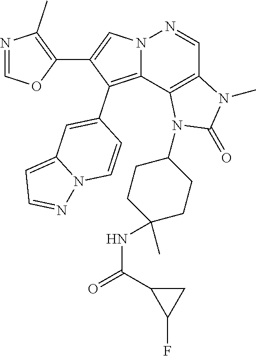 [SMILES: Cc1ncoc1-c1cn2ncc3c(c2c1-c1ccn2nccc2c1)n(C1CCC(C)(NC(=O)C2CC2F)CC1)c(=O)n3C] |
| I-206 | 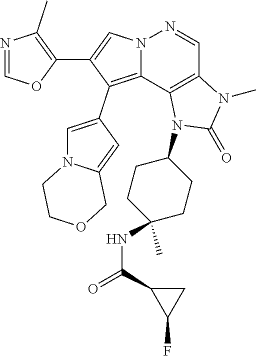 [SMILES: Cc1ncoc1-c1cn2ncc3c(c2c1-c1cc2n(c1)CCOC2)n([C@H]1CC[C@@](C)(NC(=O)[C@H]2C[C@H]2F)CC1)c(=O)n3C] |
| I-206′ | 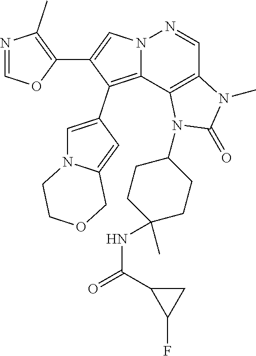 [SMILES: Cc1ncoc1-c1cn2ncc3c(c2c1-c1cc2n(c1)CCOC2)n(C1CCC(C)(NC(=O)C2CC2F)CC1)c(=O)n3C] |
| I-207 | 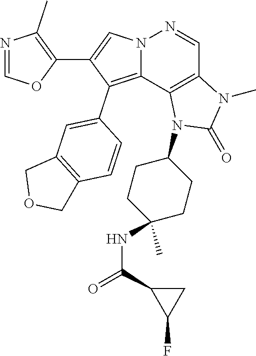 [SMILES: Cc1ncoc1-c1cn2ncc3c(c2c1-c1ccc2c(c1)COC2)n([C@H]1CC[C@@](C)(NC(=O)[C@H]2C[C@H]2F)CC1)c(=O)n3C] |
| I-207′ | 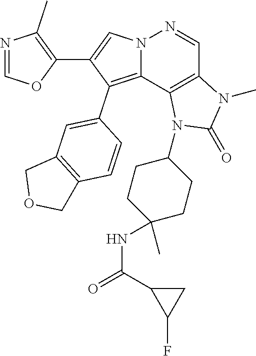 [SMILES: Cc1ncoc1-c1cn2ncc3c(c2c1-c1ccc2c(c1)COC2)n(C1CCC(C)(NC(=O)C2CC2F)CC1)c(=O)n3C] |
| I-208 | 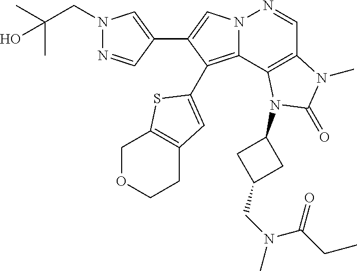 [SMILES: CCC(=O)N(C)C[C@H]1C[C@H](n2c(=O)n(C)c3cnn4cc(-c5cnn(CC(C)(C)O)c5)c(-c5cc6c(s5)COCC6)c4c32)C1] |
| I-208′ | 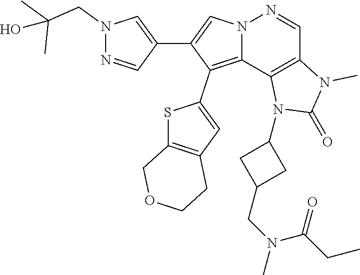 [SMILES: CCC(=O)N(C)CC1CC(n2c(=O)n(C)c3cnn4cc(-c5cnn(CC(C)(C)O)c5)c(-c5cc6c(s5)COCC6)c4c32)C1] |
| I-209 | 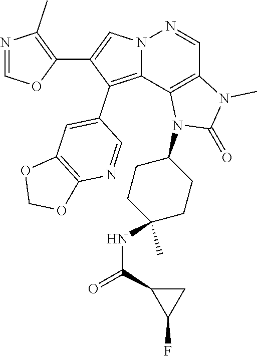 [SMILES: Cc1ncoc1-c1cn2ncc3c(c2c1-c1cnc2c(c1)OCO2)n([C@H]1CC[C@@](C)(NC(=O)[C@H]2C[C@H]2F)CC1)c(=O)n3C] |
| I-209′ | 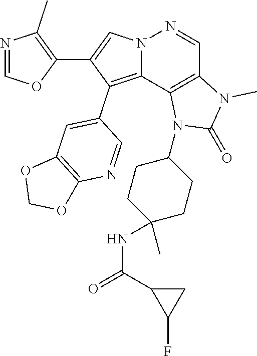 [SMILES: Cc1ncoc1-c1cn2ncc3c(c2c1-c1cnc2c(c1)OCO2)n(C1CCC(C)(NC(=O)C2CC2F)CC1)c(=O)n3C] |
| I-210 | 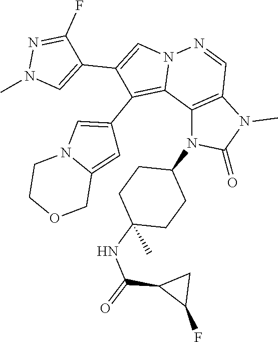 |
| I-210′ | 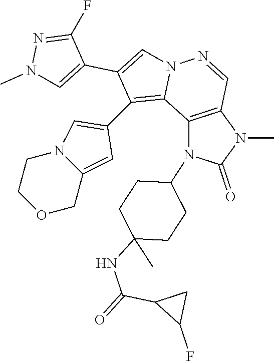 |
| I-211 | 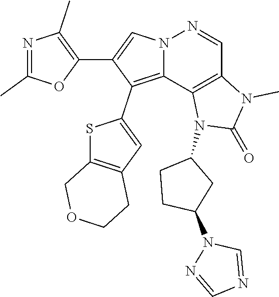 |
| I-211′ | 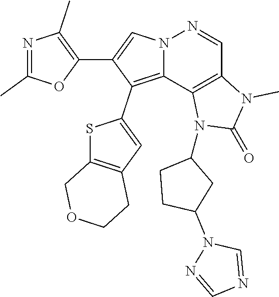 |
| I-212 | 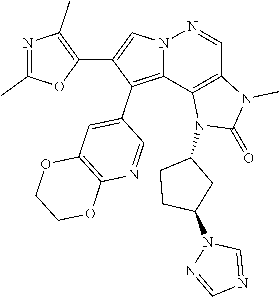 |
| I-212′ | 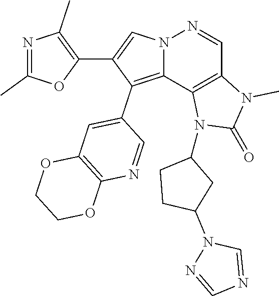 |
| I-213 | 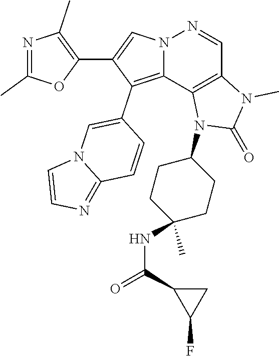 |
| I-213′ | 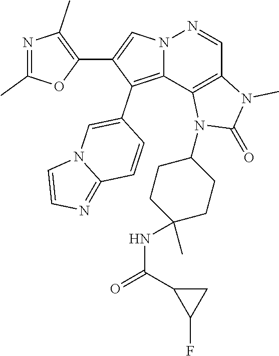 |
| I-214 | 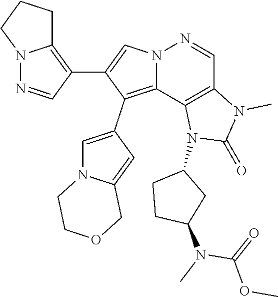 [SMILES: COC(=O)N(C)[C@H]1CC[C@H](n2c(=O)n(C)c3cnn4cc(-c5cnn6c5CCC6)c(-c5cc6n(c5)CCOC6)c4c32)C1] |
| I-214′ | 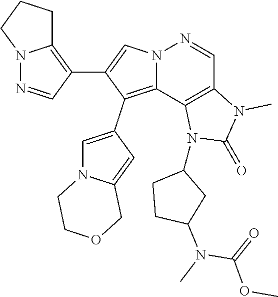 [SMILES: COC(=O)N(C)C1CCC(n2c(=O)n(C)c3cnn4cc(-c5cnn6c5CCC6)c(-c5cc6n(c5)CCOC6)c4c32)C1] |
| I-215 | 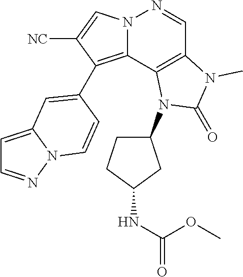 [SMILES: COC(=O)N[C@@H]1CC[C@@H](n2c(=O)n(C)c3cnn4cc(C#N)c(-c5ccn6nccc6c5)c4c32)C1] |
| I-215′ | 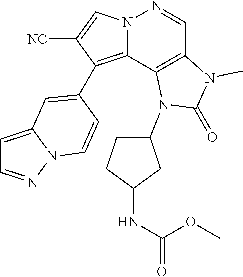 [SMILES: COC(=O)NC1CCC(n2c(=O)n(C)c3cnn4cc(C#N)c(-c5ccn6nccc6c5)c4c32)C1] |
| I-216 | 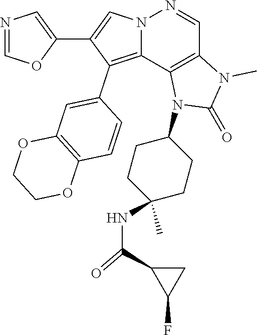 [SMILES: Cn1c(=O)n([C@H]2CC[C@@](C)(NC(=O)[C@H]3C[C@H]3F)CC2)c2c1cnn1cc(-c3cnco3)c(-c3ccc4c(c3)OCCO4)c21] |
| I-216′ | 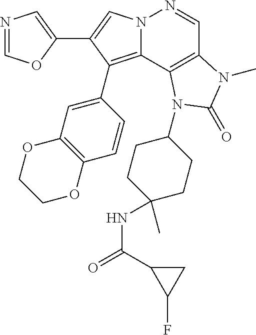 [SMILES: Cn1c(=O)n(C2CCC(C)(NC(=O)C3CC3F)CC2)c2c1cnn1cc(-c3cnco3)c(-c3ccc4c(c3)OCCO4)c21] |
| I-217 | 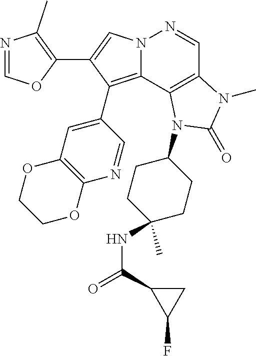 [SMILES: Cc1ncoc1-c1cn2ncc3c(c2c1-c1cnc2c(c1)OCCO2)n([C@H]1CC[C@@](C)(NC(=O)[C@H]2C[C@H]2F)CC1)c(=O)n3C] |
| I-217′ | 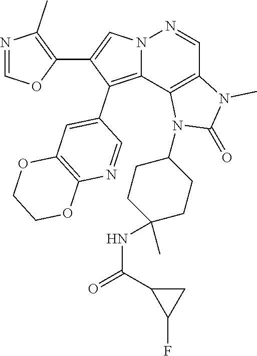 [SMILES: Cc1ncoc1-c1cn2ncc3c(c2c1-c1cnc2c(c1)OCCO2)n(C1CCC(C)(NC(=O)C2CC2F)CC1)c(=O)n3C] |
| I-218 | 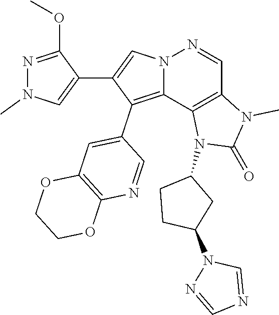 |
| I-218′ | 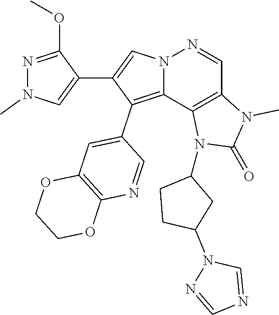 |
| I-219 | 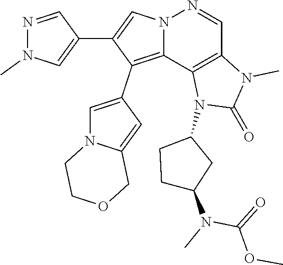 |
| I-219′ | 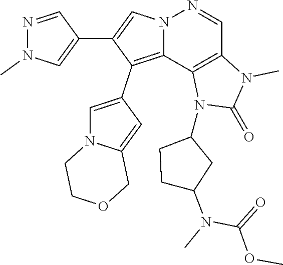 |
| I-220 | 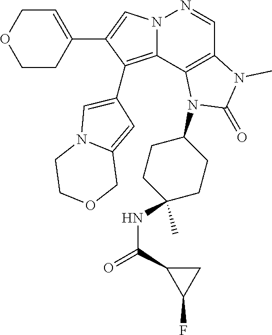 [SMILES: Cn1c(=O)n([C@H]2CC[C@@](C)(NC(=O)[C@H]3C[C@H]3F)CC2)c2c1cnn1cc(C3=CCOCC3)c(-c3cc4n(c3)CCOC4)c21] |
| I-220′ | 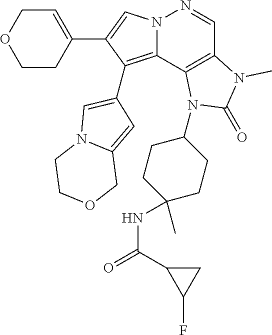 [SMILES: Cn1c(=O)n(C2CCC(C)(NC(=O)C3CC3F)CC2)c2c1cnn1cc(C3=CCOCC3)c(-c3cc4n(c3)CCOC4)c21] |
| I-221 | 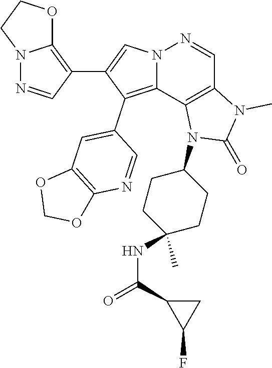 [SMILES: Cn1c(=O)n([C@H]2CC[C@@](C)(NC(=O)[C@H]3C[C@H]3F)CC2)c2c1cnn1cc(-c3cnn4c3OCC4)c(-c3cnc4c(c3)OCO4)c21] |
| I-221′ | 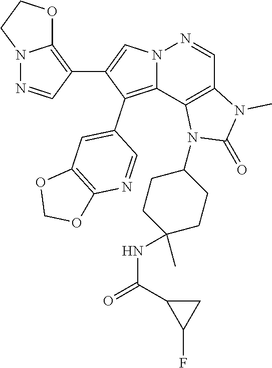 [SMILES: Cn1c(=O)n(C2CCC(C)(NC(=O)C3CC3F)CC2)c2c1cnn1cc(-c3cnn4c3OCC4)c(-c3cnc4c(c3)OCO4)c21] |
| I-222 | 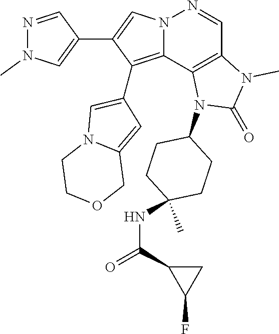 |
| I-222′ | 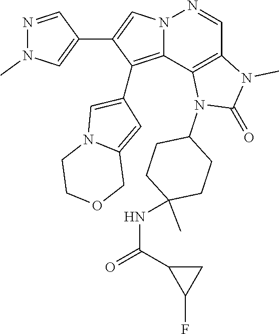 |
| I-223 | 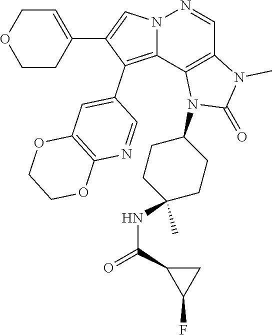 [SMILES: Cn1c(=O)n([C@H]2CC[C@@](C)(NC(=O)[C@H]3C[C@H]3F)CC2)c2c1cnn1cc(C3=CCOCC3)c(-c3cnc4c(c3)OCCO4)c21] |
| I-223′ | 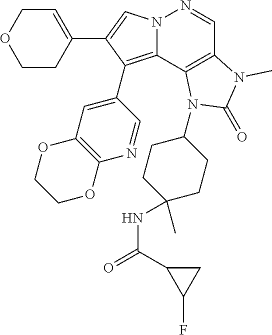 [SMILES: Cn1c(=O)n(C2CCC(C)(NC(=O)C3CC3F)CC2)c2c1cnn1cc(C3=CCOCC3)c(-c3cnc4c(c3)OCCO4)c21] |
| I-224 | 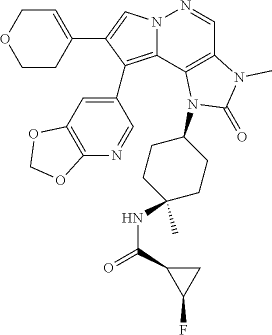 [SMILES: Cn1c(=O)n([C@H]2CC[C@@](C)(NC(=O)[C@H]3C[C@H]3F)CC2)c2c1cnn1cc(C3=CCOCC3)c(-c3cnc4c(c3)OCO4)c21] |
| I-224′ | 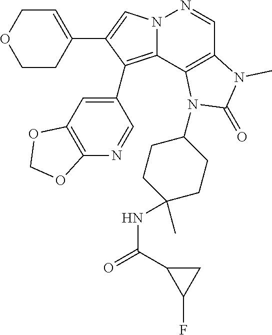 [SMILES: Cn1c(=O)n(C2CCC(C)(NC(=O)C3CC3F)CC2)c2c1cnn1cc(C3=CCOCC3)c(-c3cnc4c(c3)OCO4)c21] |
| I-225 | 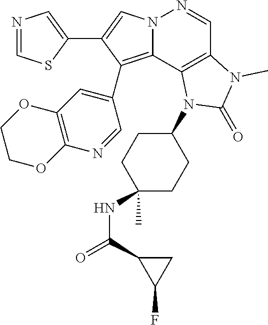 [SMILES: Cn1c(=O)n([C@H]2CC[C@@](C)(NC(=O)[C@H]3C[C@H]3F)CC2)c2c1cnn1cc(-c3cncs3)c(-c3cnc4c(c3)OCCO4)c21] |
| I-225′ | 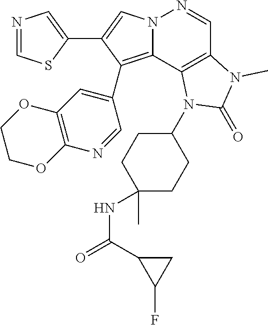 [SMILES: Cn1c(=O)n(C2CCC(C)(NC(=O)C3CC3F)CC2)c2c1cnn1cc(-c3cncs3)c(-c3cnc4c(c3)OCCO4)c21] |
| I-226 | 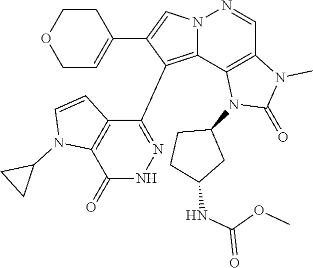 [SMILES: COC(=O)N[C@@H]1CC[C@@H](n2c(=O)n(C)c3cnn4cc(C5=CCOCC5)c(-c5n[nH]c(=O)c6c5ccn6C5CC5)c4c32)C1] |
| I-226′ | 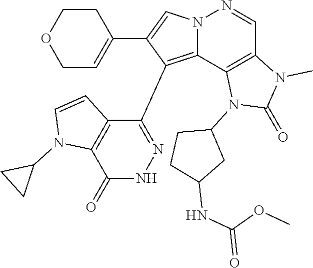 [SMILES: COC(=O)NC1CCC(n2c(=O)n(C)c3cnn4cc(C5=CCOCC5)c(-c5n[nH]c(=O)c6c5ccn6C5CC5)c4c32)C1] |
| I-227 | 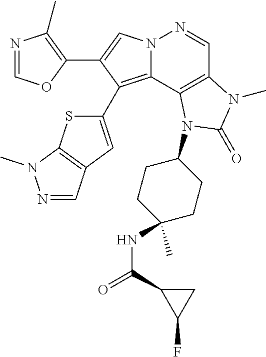 |
| I-227′ | 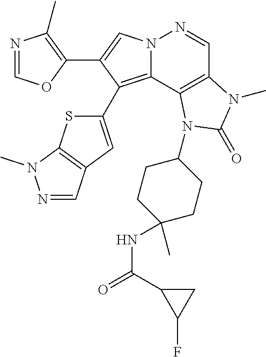 |
| I-228 | 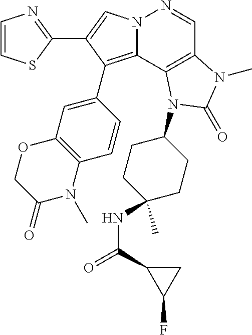 [SMILES: CN1C(=O)COc2cc(-c3c(-c4nccs4)cn4ncc5c(c34)n([C@H]3CC[C@@](C)(NC(=O)[C@H]4C[C@H]4F)CC3)c(=O)n5C)ccc21] |
| I-228′ | 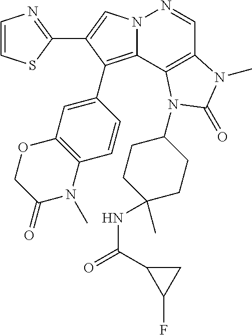 [SMILES: CN1C(=O)COc2cc(-c3c(-c4nccs4)cn4ncc5c(c34)n(C3CCC(C)(NC(=O)C4CC4F)CC3)c(=O)n5C)ccc21] |
| I-229 | 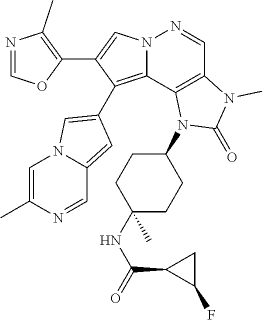 |
| I-229′ | 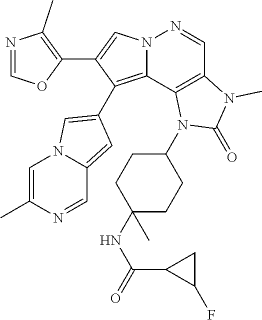 |
| I-230 | 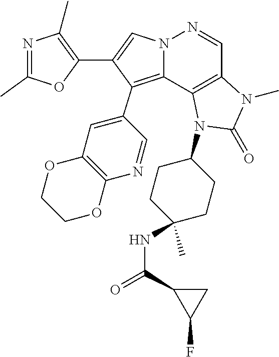 |
| I-230′ | 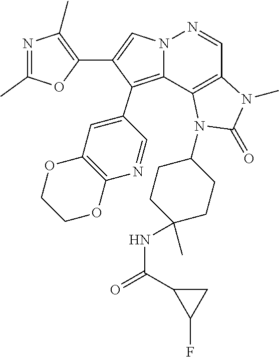 |
| I-231 | 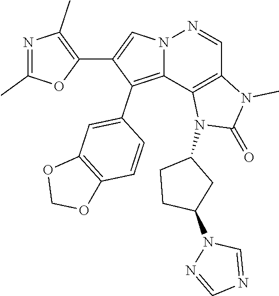 |
| I-231′ | 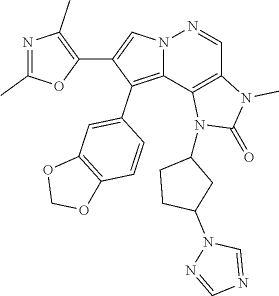 |
| I-232 | 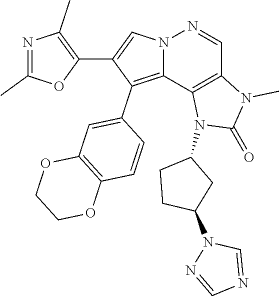 |
| I-232′ | 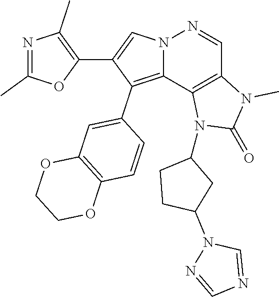 |
| I-233 | 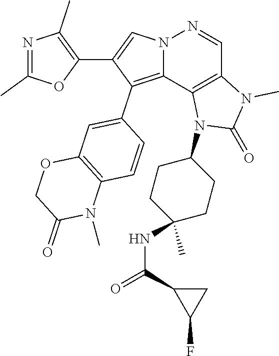 |
| I-233′ | 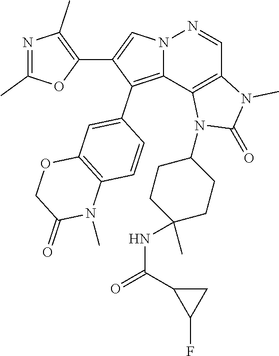 |
| I-234 | 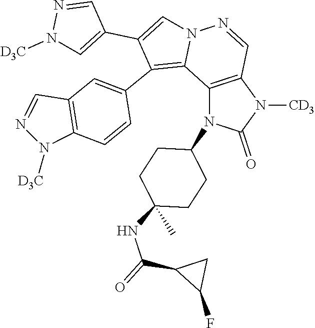 [SMILES: [2H]C([2H])([2H])n1cc(-c2cn3ncc4c(c3c2-c2ccc3c(cnn3C([2H])([2H])[2H])c2)n([C@H]2CC[C@@](C)(NC(=O)[C@H]3C[C@H]3F)CC2)c(=O)n4C([2H])([2H])[2H])cn1] |
| I-234′ | 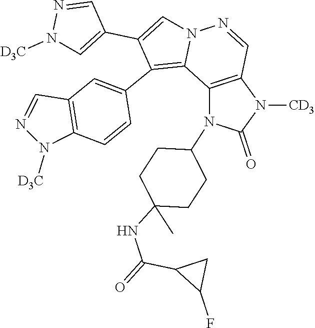 [SMILES: [2H]C([2H])([2H])n1cc(-c2cn3ncc4c(c3c2-c2ccc3c(cnn3C([2H])([2H])[2H])c2)n(C2CCC(C)(NC(=O)C3CC3F)CC2)c(=O)n4C([2H])([2H])[2H])cn1] |
| I-235 | 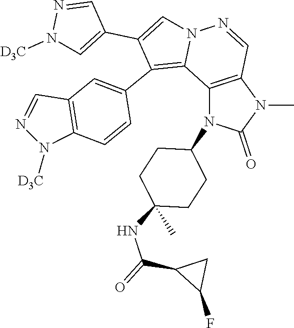 [SMILES: [2H]C([2H])([2H])n1cc(-c2cn3ncc4c(c3c2-c2ccc3c(cnn3C([2H])([2H])[2H])c2)n([C@H]2CC[C@@](C)(NC(=O)[C@H]3C[C@H]3F)CC2)c(=O)n4C)cn1] |
| I-235′ | 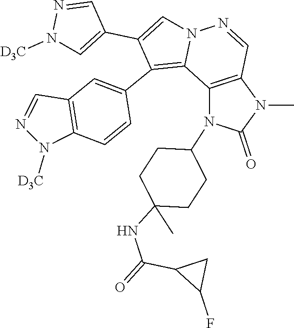 [SMILES: [2H]C([2H])([2H])n1cc(-c2cn3ncc4c(c3c2-c2ccc3c(cnn3C([2H])([2H])[2H])c2)n(C2CCC(C)(NC(=O)C3CC3F)CC2)c(=O)n4C)cn1] |
| I-236 | 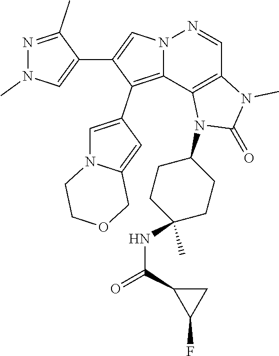 |
| I-236′ | 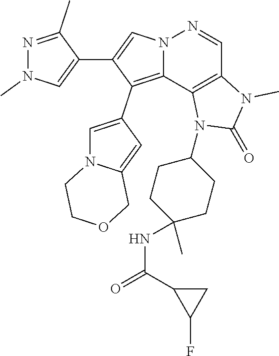 |
| I-237 | 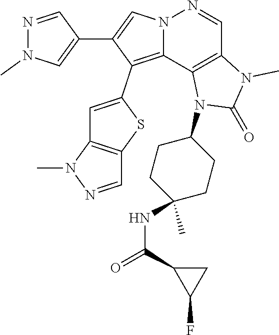 |
| I-237′ | 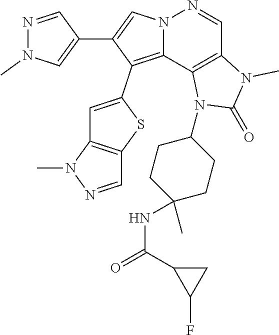 |
| I-238 | 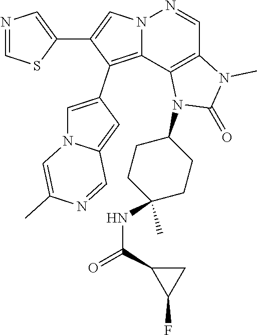 [SMILES: Cc1cn2cc(-c3c(-c4cncs4)cn4ncc5c(c34)n([C@H]3CC[C@@](C)(NC(=O)[C@H]4C[C@H]4F)CC3)c(=O)n5C)cc2cn1] |
| I-238′ | 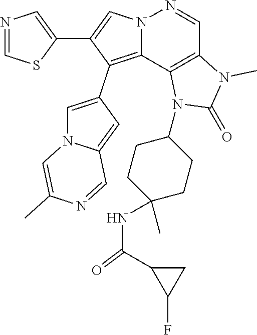 [SMILES: Cc1cn2cc(-c3c(-c4cncs4)cn4ncc5c(c34)n(C3CCC(C)(NC(=O)C4CC4F)CC3)c(=O)n5C)cc2cn1] |
| I-239 | 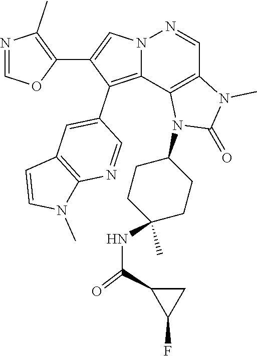 [SMILES: Cc1ncoc1-c1cn2ncc3c(c2c1-c1cnc2c(ccn2C)c1)n([C@H]1CC[C@@](C)(NC(=O)[C@H]2C[C@H]2F)CC1)c(=O)n3C] |
| I-239′ | 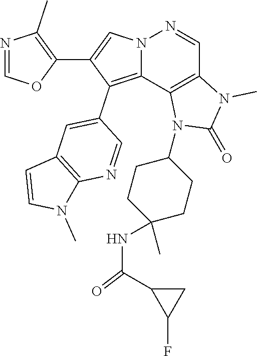 [SMILES: Cc1ncoc1-c1cn2ncc3c(c2c1-c1cnc2c(ccn2C)c1)n(C1CCC(C)(NC(=O)C2CC2F)CC1)c(=O)n3C] |
| I-240 | 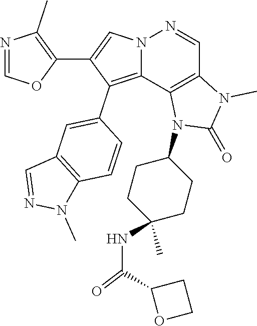 [SMILES: Cc1ncoc1-c1cn2ncc3c(c2c1-c1ccc2c(cnn2C)c1)n([C@H]1CC[C@@](C)(NC(=O)[C@@H]2CCO2)CC1)c(=O)n3C] |
| I-240′ | 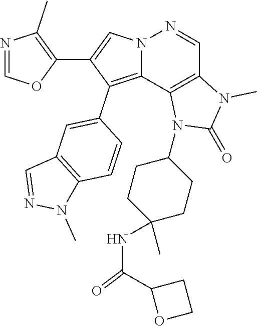 [SMILES: Cc1ncoc1-c1cn2ncc3c(c2c1-c1ccc2c(cnn2C)c1)n(C1CCC(C)(NC(=O)C2CCO2)CC1)c(=O)n3C] |
| I-241 | 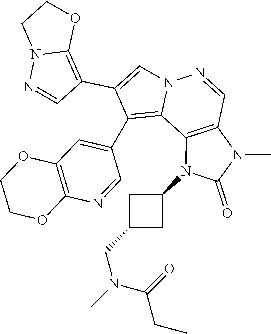 [SMILES: CCC(=O)N(C)C[C@H]1C[C@H](n2c(=O)n(C)c3cnn4cc(-c5cnn6c5OCC6)c(-c5cnc6c(c5)OCCO6)c4c32)C1] |
| I-241′ | 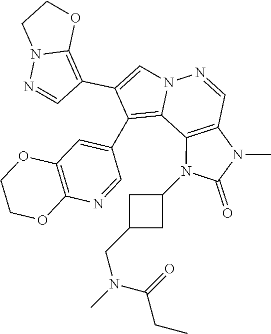 [SMILES: CCC(=O)N(C)CC1CC(n2c(=O)n(C)c3cnn4cc(-c5cnn6c5OCC6)c(-c5cnc6c(c5)OCCO6)c4c32)C1] |
| I-242 | 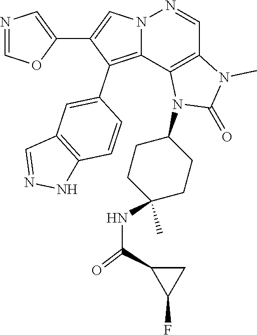 [SMILES: Cn1c(=O)n([C@H]2CC[C@@](C)(NC(=O)[C@H]3C[C@H]3F)CC2)c2c1cnn1cc(-c3cnco3)c(-c3ccc4[nH]ncc4c3)c21] |
| I-242′ | 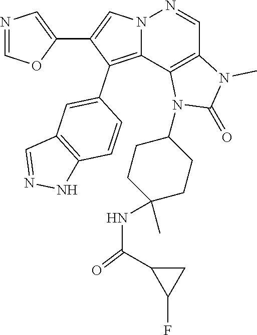 [SMILES: Cn1c(=O)n(C2CCC(C)(NC(=O)C3CC3F)CC2)c2c1cnn1cc(-c3cnco3)c(-c3ccc4[nH]ncc4c3)c21] |
| I-243-1 | 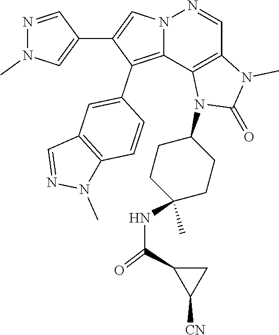 |
| I-243-2 | 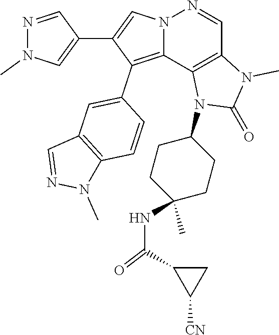 |
| I-243′ | 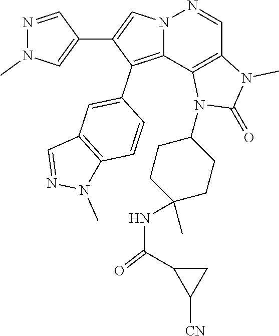 |
| I-244 | 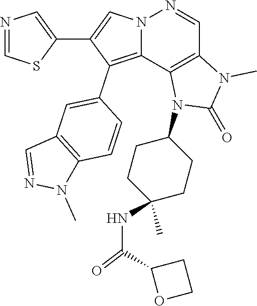 [SMILES: Cn1ncc2cc(-c3c(-c4cncs4)cn4ncc5c(c34)n([C@H]3CC[C@@](C)(NC(=O)[C@@H]4CCO4)CC3)c(=O)n5C)ccc21] |
| I-244′ | 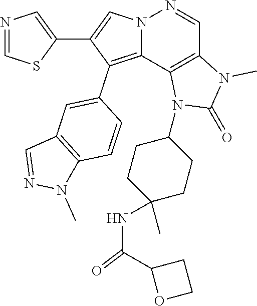 [SMILES: Cn1ncc2cc(-c3c(-c4cncs4)cn4ncc5c(c34)n(C3CCC(C)(NC(=O)C4CCO4)CC3)c(=O)n5C)ccc21] |
| I-245 | 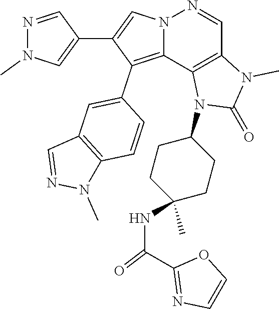 |
| I-245′ | 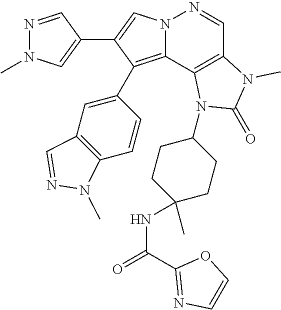 |
| I-246-1 | 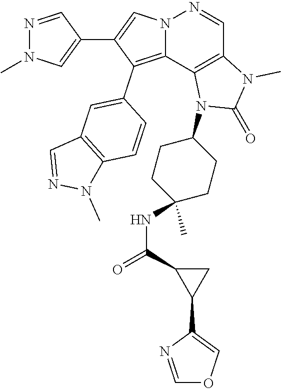 |
| I-246-2 | 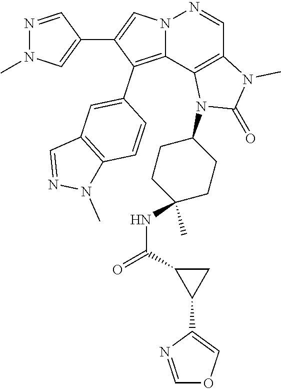 |
| I-246′ | 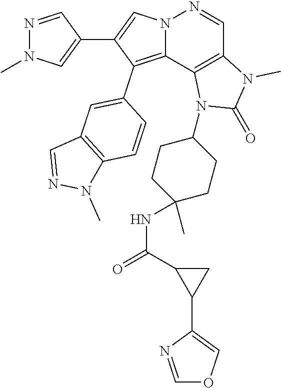 |
| I-247 | 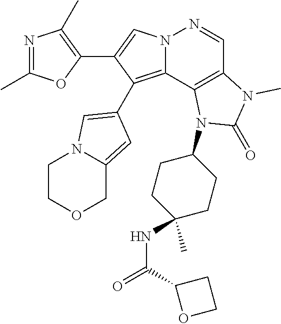 |
| I-247′ | 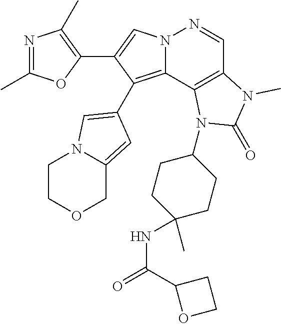 |
| I-248 | 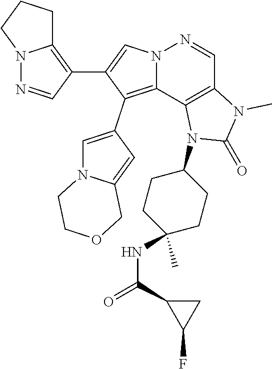 [SMILES: Cn1c(=O)n([C@H]2CC[C@@](C)(NC(=O)[C@H]3C[C@H]3F)CC2)c2c1cnn1cc(-c3cnn4c3CCC4)c(-c3cc4n(c3)CCOC4)c21] |
| I-248′ | 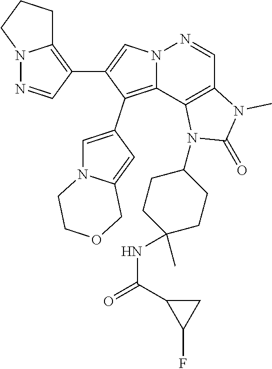 [SMILES: Cn1c(=O)n(C2CCC(C)(NC(=O)C3CC3F)CC2)c2c1cnn1cc(-c3cnn4c3CCC4)c(-c3cc4n(c3)CCOC4)c21] |
| I-249 | 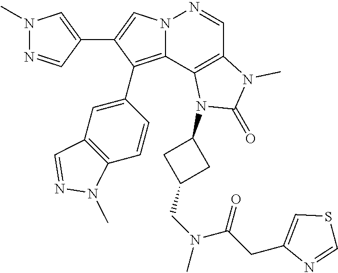 [SMILES: CN(C[C@H]1C[C@H](n2c(=O)n(C)c3cnn4cc(-c5cnn(C)c5)c(-c5ccc6c(cnn6C)c5)c4c32)C1)C(=O)Cc1cscn1] |
| I-249′ | 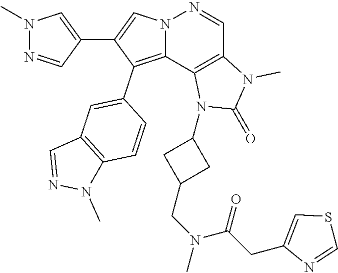 [SMILES: CN(CC1CC(n2c(=O)n(C)c3cnn4cc(-c5cnn(C)c5)c(-c5ccc6c(cnn6C)c5)c4c32)C1)C(=O)Cc1cscn1] |
| I-250 | 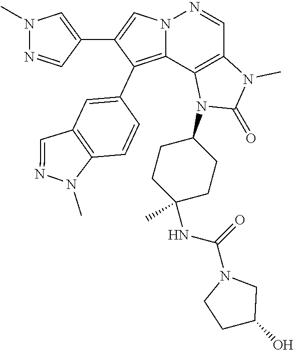 |
| I-250′ | 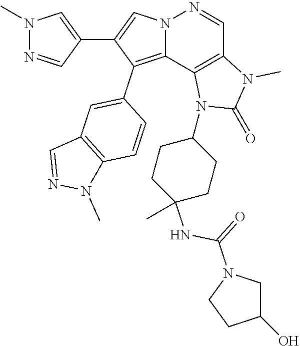 [SMILES: Cn1cc(-c2cn3ncc4c(c3c2-c2ccc3c(cnn3C)c2)n(C2CCC(C)(NC(=O)N3CCC(O)C3)CC2)c(=O)n4C)cn1] |
| I-251 | 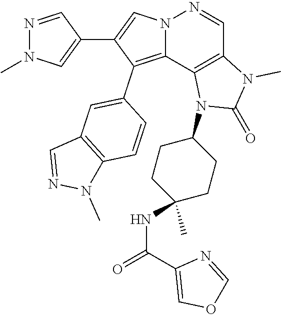 |
| I-251′ | 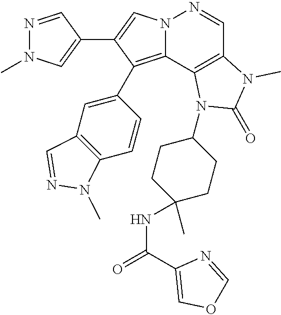 |
| I-252-1 | 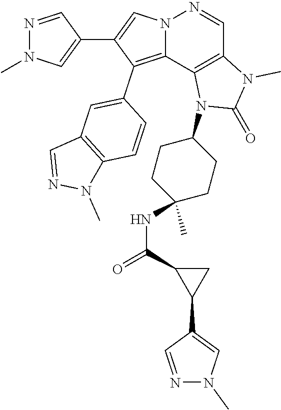 |
| I-252-2 | 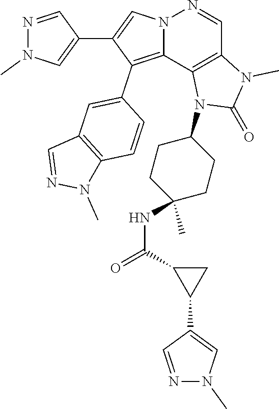 |
| I-252′ | 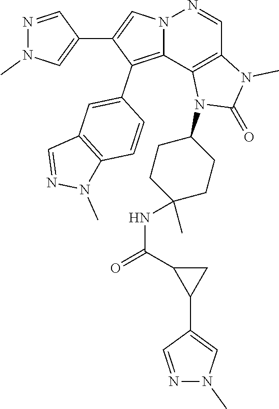 |
| I-253 | 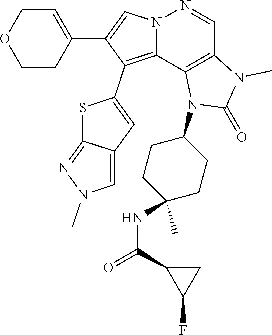 [SMILES: Cn1cc2cc(-c3c(C4=CCOCC4)cn4ncc5c(c34)n([C@H]3CC[C@@](C)(NC(=O)[C@H]4C[C@H]4F)CC3)c(=O)n5C)sc2n1] |
| I-253′ | 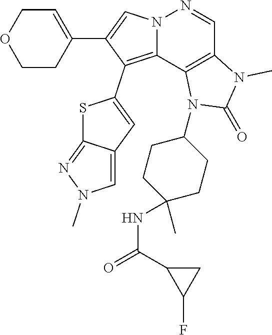 [SMILES: Cn1cc2cc(-c3c(C4=CCOCC4)cn4ncc5c(c34)n(C3CCC(C)(NC(=O)C4CC4F)CC3)c(=O)n5C)sc2n1] |
| I-254 | 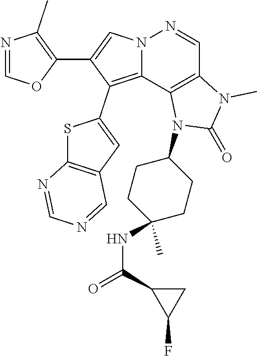 [SMILES: Cc1ncoc1-c1cn2ncc3c(c2c1-c1cc2cncnc2s1)n([C@H]1CC[C@@](C)(NC(=O)[C@H]2C[C@H]2F)CC1)c(=O)n3C] |
| I-254′ | 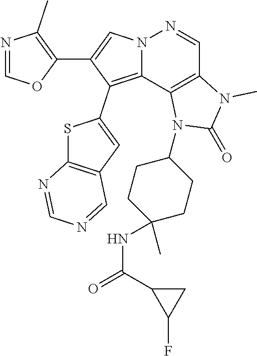 [SMILES: Cc1ncoc1-c1cn2ncc3c(c2c1-c1cc2cncnc2s1)n(C1CCC(C)(NC(=O)C2CC2F)CC1)c(=O)n3C] |
| I-255 | 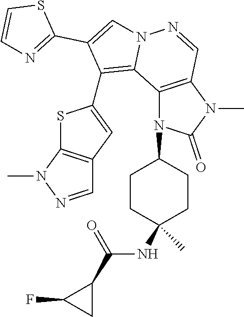 [SMILES: Cn1ncc2cc(-c3c(-c4nccs4)cn4ncc5c(c34)n([C@H]3CC[C@@](C)(NC(=O)[C@H]4C[C@H]4F)CC3)c(=O)n5C)sc21] |
| I-255′ | 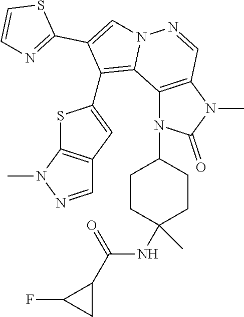 [SMILES: Cn1ncc2cc(-c3c(-c4nccs4)cn4ncc5c(c34)n(C3CCC(C)(NC(=O)C4CC4F)CC3)c(=O)n5C)sc21] |
| I-256 | 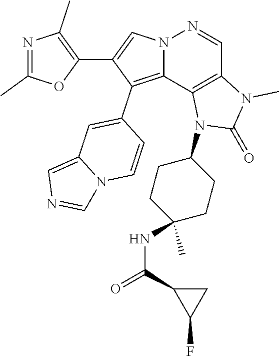 |
| I-256′ | 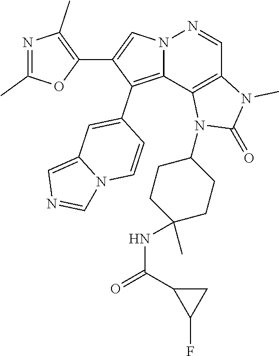 |
| I-257 | 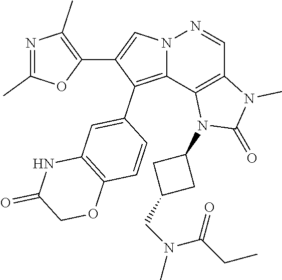 |
| I-257′ | 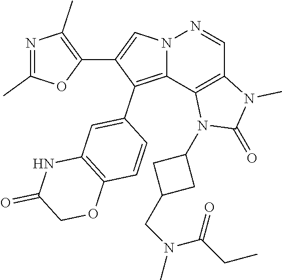 |
| I-258 | 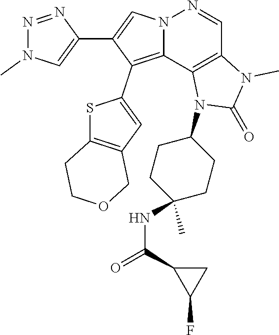 |
| I-258′ | 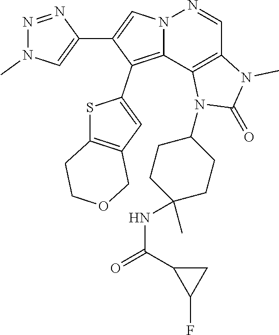 |
| I-259 | 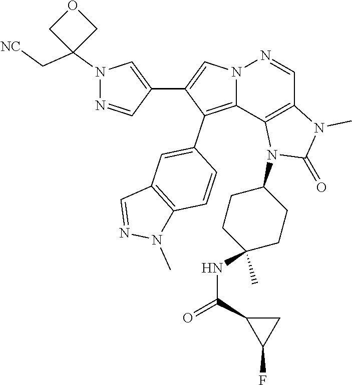 [SMILES: Cn1ncc2cc(-c3c(-c4cnn(C5(CC#N)COC5)c4)cn4ncc5c(c34)n([C@H]3CC[C@@](C)(NC(=O)[C@H]4C[C@H]4F)CC3)c(=O)n5C)ccc21] |
| I-259′ | 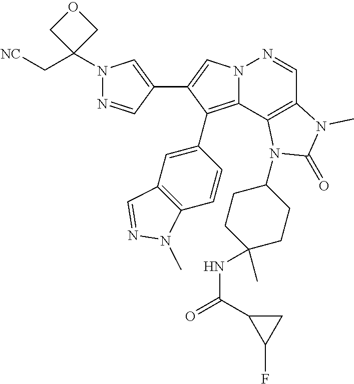 |
| I-260 | 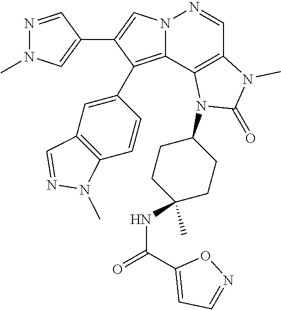 |
| I-260′ | 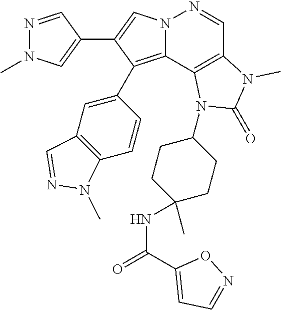 |
| I-261 | 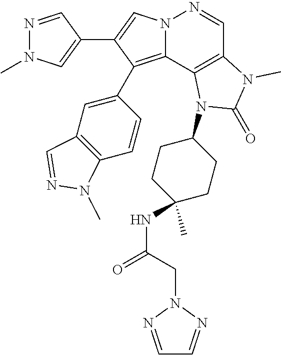 |
| I-261′ | 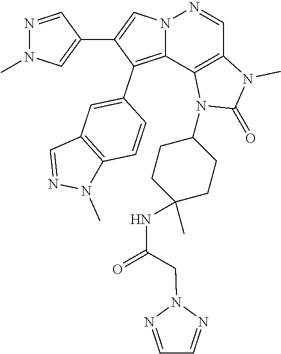 |
| I-262-1 | 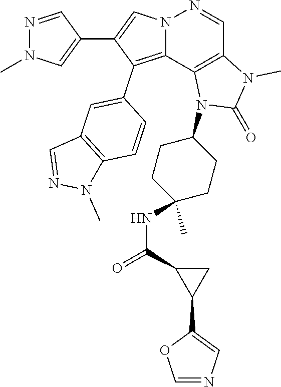 |
| I-262-2 | 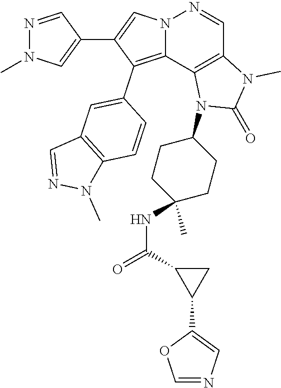 |
| I-262′ | 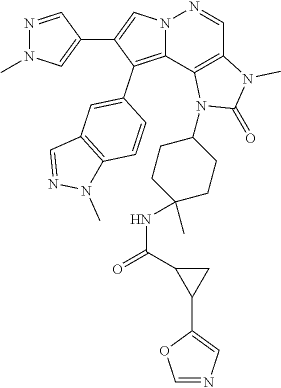 |
| I-263 | 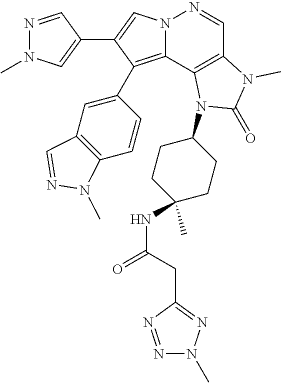 |
| I-263′ | 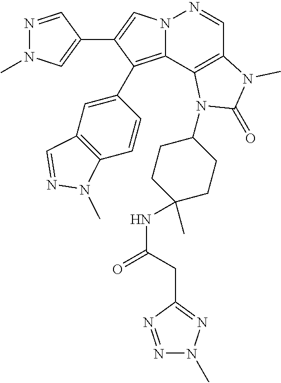 |
| I-264 | 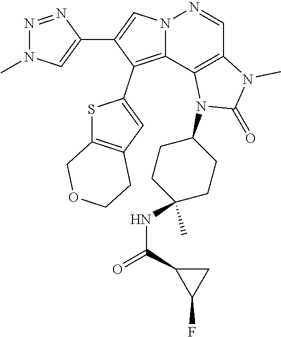 |
| I-264′ | 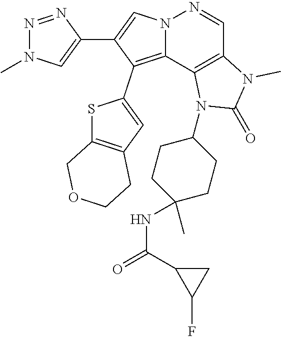 |
| I-265 | 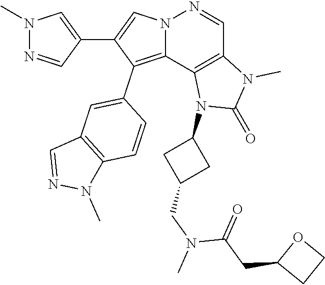 [SMILES: CN(C[C@H]1C[C@H](n2c(=O)n(C)c3cnn4cc(-c5cnn(C)c5)c(-c5ccc6c(cnn6C)c5)c4c32)C1)C(=O)C[C@@H]1CCO1] |
| I-265′ | 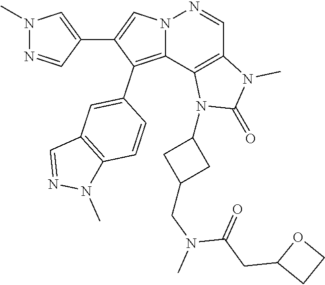 [SMILES: CN(CC1CC(n2c(=O)n(C)c3cnn4cc(-c5cnn(C)c5)c(-c5ccc6c(cnn6C)c5)c4c32)C1)C(=O)CC1CCO1] |
| I-266 | 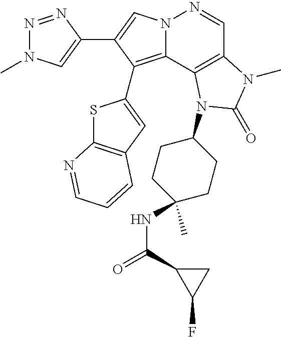 |
| I-266′ | 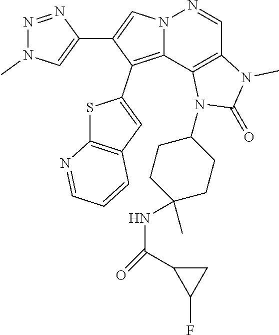 |
| I-267 | 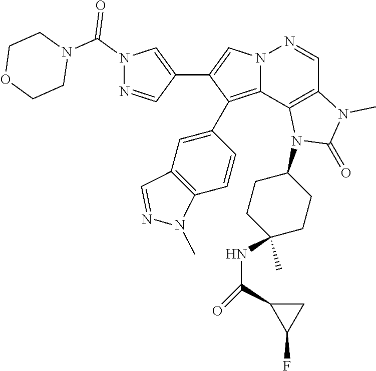 [SMILES: Cn1ncc2cc(-c3c(-c4cnn(C(=O)N5CCOCC5)c4)cn4ncc5c(c34)n([C@H]3CC[C@@](C)(NC(=O)[C@H]4C[C@H]4F)CC3)c(=O)n5C)ccc21] |
| I-267′ | 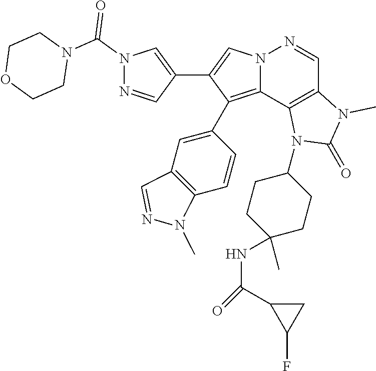 [SMILES: Cn1ncc2cc(-c3c(-c4cnn(C(=O)N5CCOCC5)c4)cn4ncc5c(c34)n(C3CCC(C)(NC(=O)C4CC4F)CC3)c(=O)n5C)ccc21] |
| I-268 | 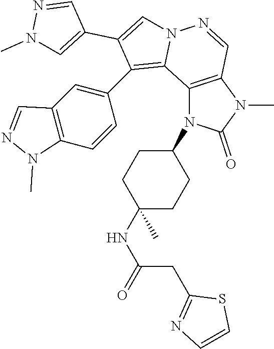 |
| I-268′ | 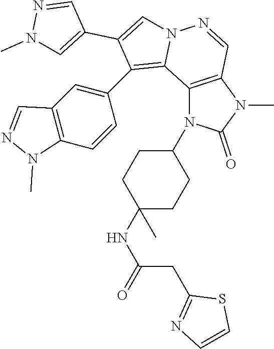 |
| I-269 | 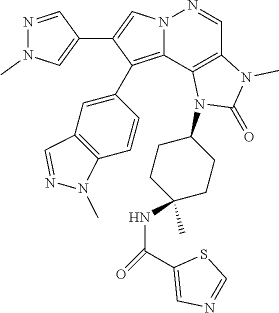 |
| I-269′ | 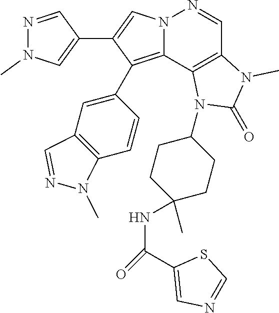 |
| I-270 | 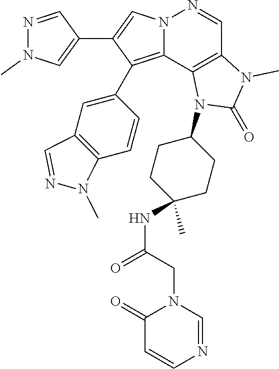 |
| I-270′ | 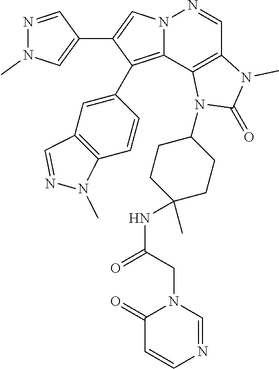 |
| I-271 | 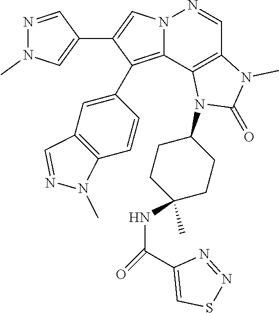 |
| I-271′ | 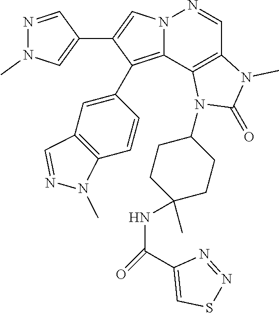 |
| I-272 | 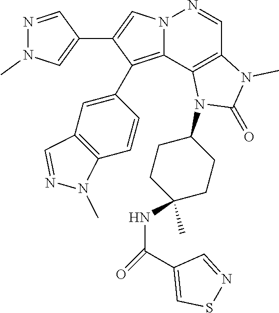 |
| I-272′ | 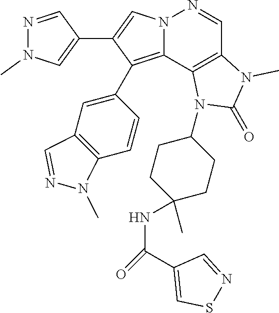 |
| I-273 | 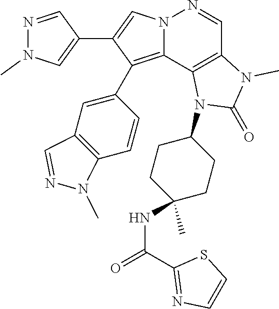 |
| I-273′ | 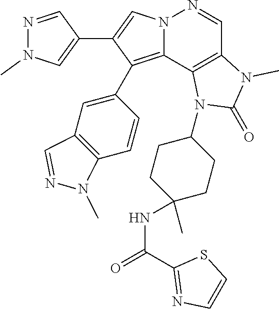 |
| I-274 | 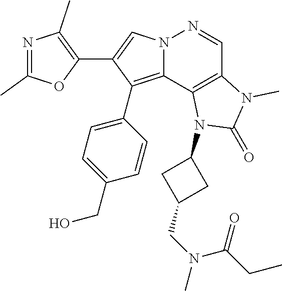 [SMILES: CCC(=O)N(C)C[C@H]1C[C@H](n2c(=O)n(C)c3cnn4cc(-c5oc(C)nc5C)c(-c5ccc(CO)cc5)c4c32)C1] |
| I-274′ | 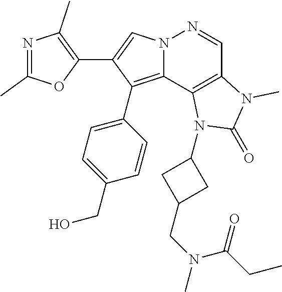 [SMILES: CCC(=O)N(C)CC1CC(n2c(=O)n(C)c3cnn4cc(-c5oc(C)nc5C)c(-c5ccc(CO)cc5)c4c32)C1] |
| I-275 | 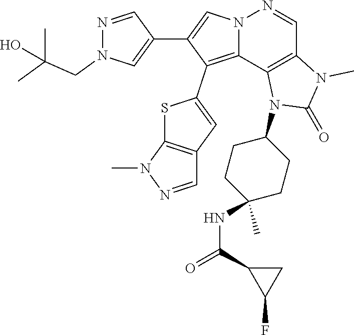 [SMILES: Cn1ncc2cc(-c3c(-c4cnn(CC(C)(C)O)c4)cn4ncc5c(c34)n([C@H]3CC[C@@](C)(NC(=O)[C@H]4C[C@H]4F)CC3)c(=O)n5C)sc21] |
| I-275′ | 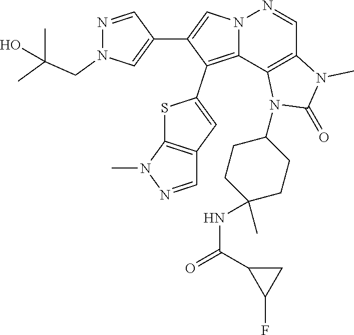 [SMILES: Cn1ncc2cc(-c3c(-c4cnn(CC(C)(C)O)c4)cn4ncc5c(c34)n(C3CCC(C)(NC(=O)C4CC4F)CC3)c(=O)n5C)sc21] |
| I-276 | 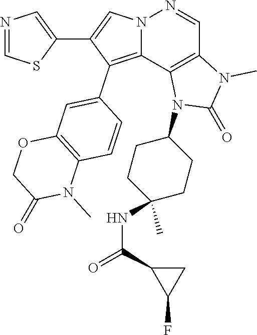 [SMILES: CN1C(=O)COc2cc(-c3c(-c4cncs4)cn4ncc5c(c34)n([C@H]3CC[C@@](C)(NC(=O)[C@H]4C[C@H]4F)CC3)c(=O)n5C)ccc21] |
| I-276′ | 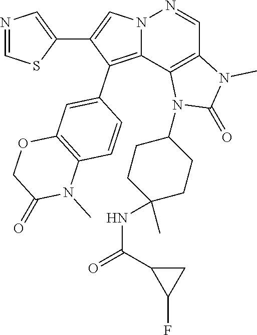 [SMILES: CN1C(=O)COc2cc(-c3c(-c4cncs4)cn4ncc5c(c34)n(C3CCC(C)(NC(=O)C4CC4F)CC3)c(=O)n5C)ccc21] |
| I-277 | 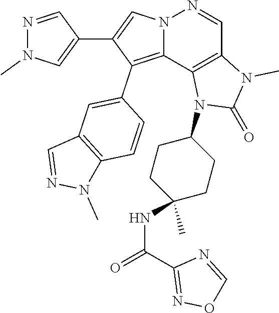 |
| I-277′ | 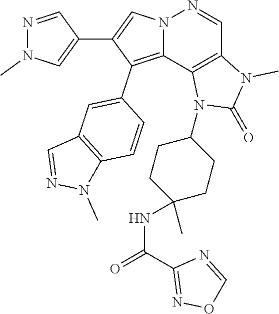 |
| I-278 | 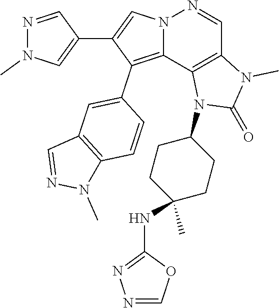 |
| I-278′ | 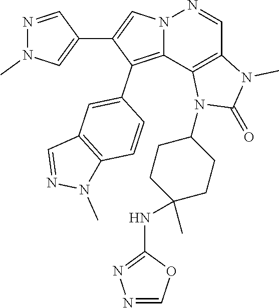 |
| I-279 | 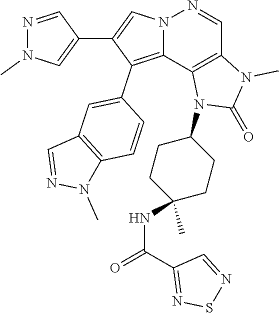 |
| I-279′ | 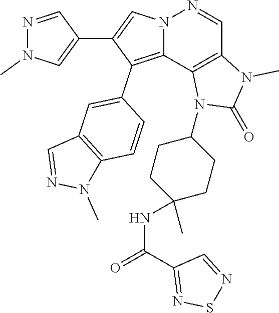 |
| I-280 | 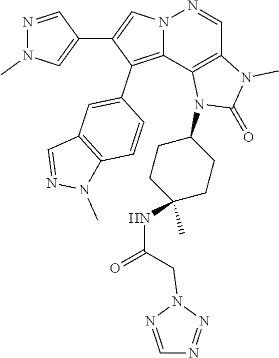 |
| I-280′ | 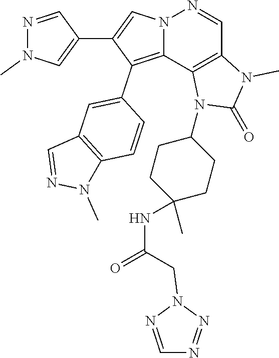 |
| I-281 | 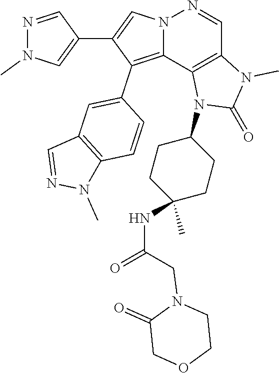 |
| I-281′ | 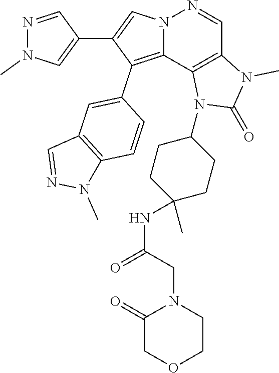 |
| I-282-1 | 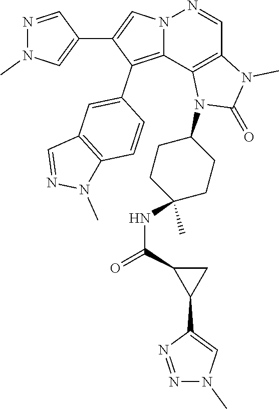 |
| I-282-2 | 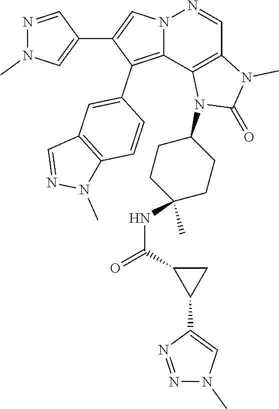 |
| I-282′ | 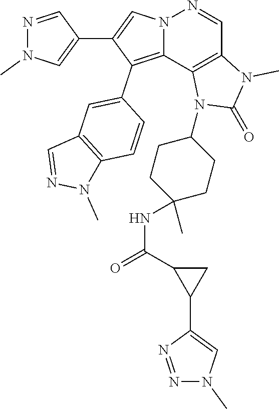 |
| I-283-1 | 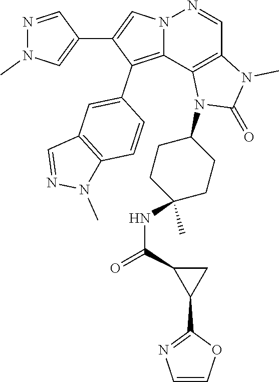 |
| I-283-2 | 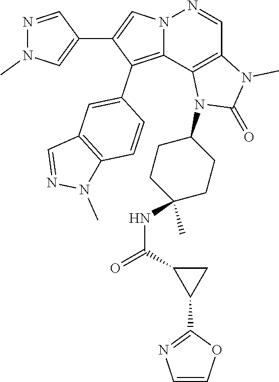 |
| I-283′ | 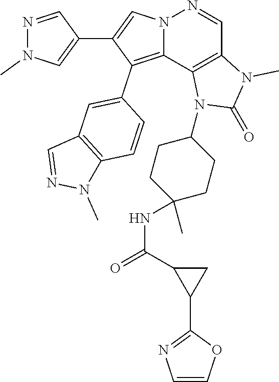 |
| I-284 | 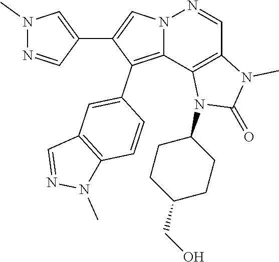 |
| I-284′ | 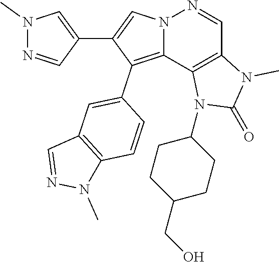 |
| I-285 | 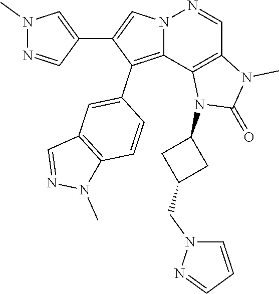 |
| I-285′ | 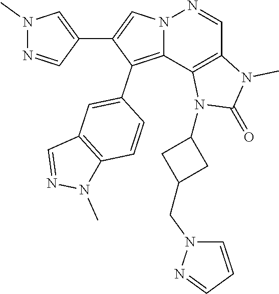 |
| I-286 | 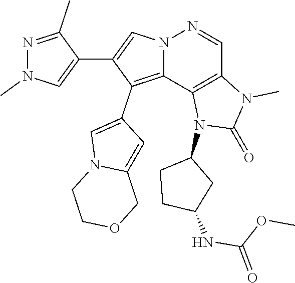 |
| I-286′ | 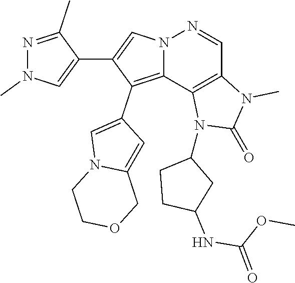 |
| I-287 | 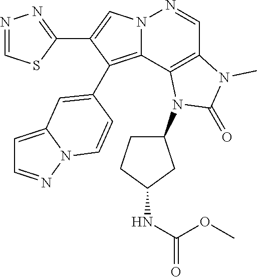 [SMILES: COC(=O)N[C@@H]1CC[C@@H](n2c(=O)n(C)c3cnn4cc(-c5nncs5)c(-c5ccn6nccc6c5)c4c32)C1] |
| I-287′ | 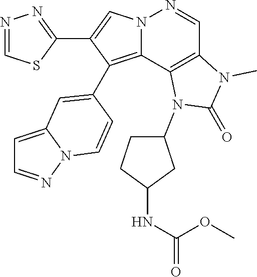 [SMILES: COC(=O)NC1CCC(n2c(=O)n(C)c3cnn4cc(-c5nncs5)c(-c5ccn6nccc6c5)c4c32)C1] |
| I-288 | 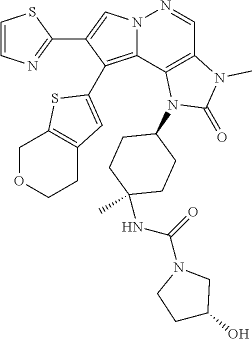 [SMILES: Cn1c(=O)n([C@H]2CC[C@@](C)(NC(=O)N3CC[C@@H](O)C3)CC2)c2c1cnn1cc(-c3nccs3)c(-c3cc4c(s3)COCC4)c21] |
| I-288′ | 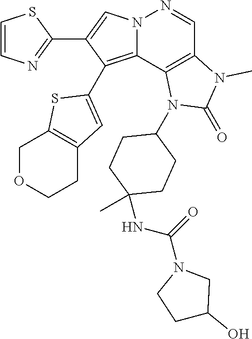 [SMILES: Cn1c(=O)n(C2CCC(C)(NC(=O)N3CCC(O)C3)CC2)c2c1cnn1cc(-c3nccs3)c(-c3cc4c(s3)COCC4)c21] |
| I-289 | 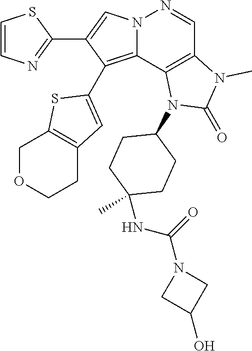 [SMILES: Cn1c(=O)n([C@H]2CC[C@@](C)(NC(=O)N3CC(O)C3)CC2)c2c1cnn1cc(-c3nccs3)c(-c3cc4c(s3)COCC4)c21] |
| I-289′ | 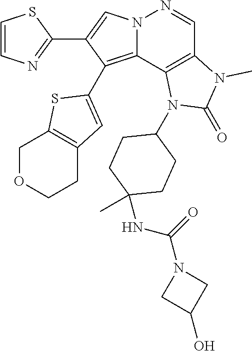 [SMILES: Cn1c(=O)n(C2CCC(C)(NC(=O)N3CC(O)C3)CC2)c2c1cnn1cc(-c3nccs3)c(-c3cc4c(s3)COCC4)c21] |
| I-290 | 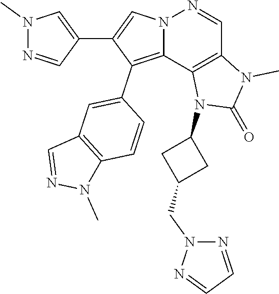 |
| I-290′ | 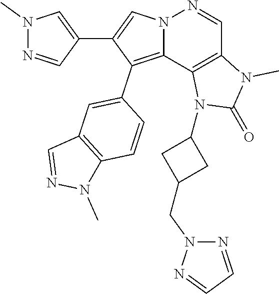 |
| I-291 | 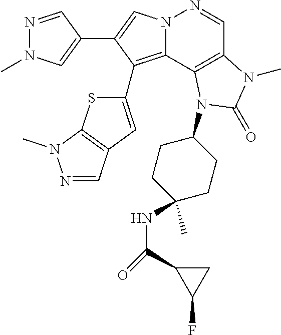 |
| I-291′ | 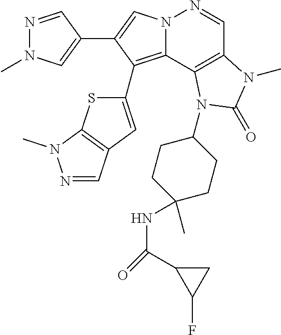 |
| I-292 | 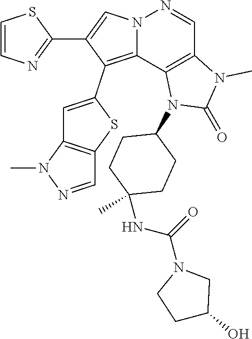 [SMILES: Cn1ncc2sc(-c3c(-c4nccs4)cn4ncc5c(c34)n([C@H]3CC[C@@](C)(NC(=O)N4CC[C@@H](O)C4)CC3)c(=O)n5C)cc21] |
| I-292′ | 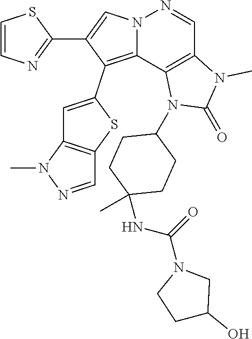 [SMILES: Cn1ncc2sc(-c3c(-c4nccs4)cn4ncc5c(c34)n(C3CCC(C)(NC(=O)N4CCC(O)C4)CC3)c(=O)n5C)cc21] |
| I-293 | 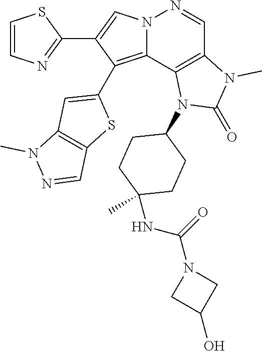 |
| I-293′ | 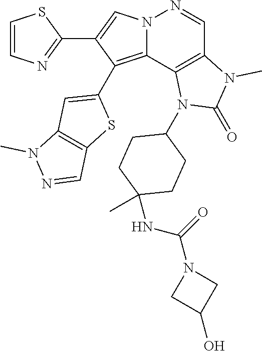 |
| I-294 | 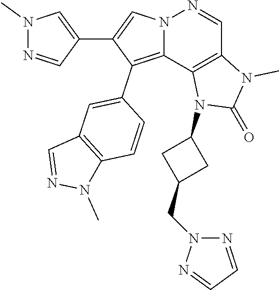 |
| I-294′ | 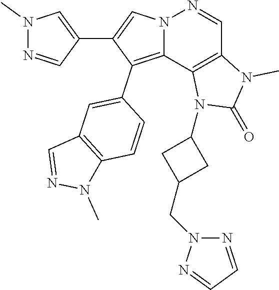 |
| I-295 | 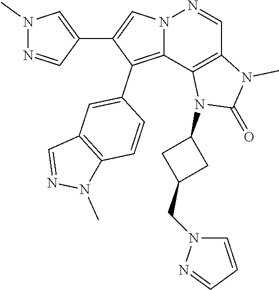 |
| I-295′ | 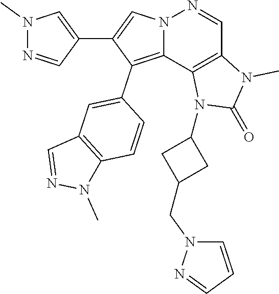 |
| I-296 | 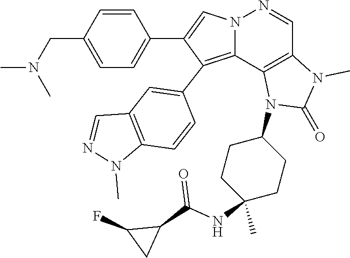 |
| I-296′ | 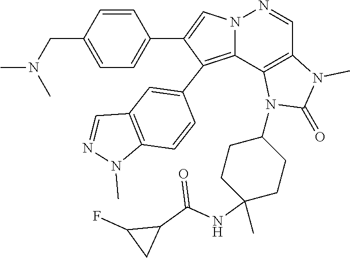 |
| I-297-1 | 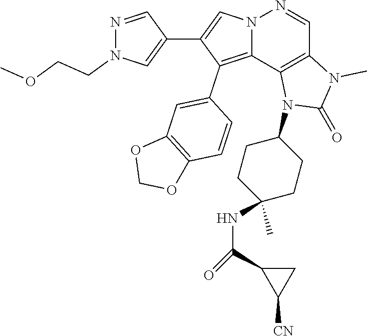 |
| I-297-2 | 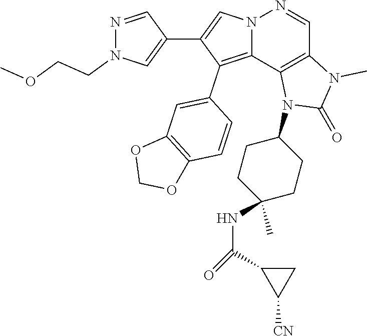 |
| I-297′ | 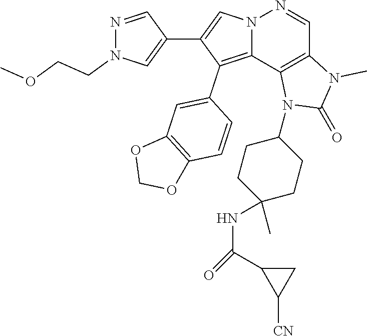 |
| I-298 | 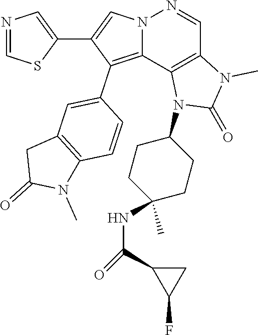 [SMILES: CN1C(=O)Cc2cc(-c3c(-c4cncs4)cn4ncc5c(c34)n([C@H]3CC[C@@](C)(NC(=O)[C@H]4C[C@H]4F)CC3)c(=O)n5C)ccc21] |
| I-298′ | 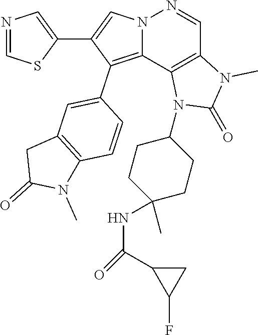 [SMILES: CN1C(=O)Cc2cc(-c3c(-c4cncs4)cn4ncc5c(c34)n(C3CCC(C)(NC(=O)C4CC4F)CC3)c(=O)n5C)ccc21] |
| I-299-1 | 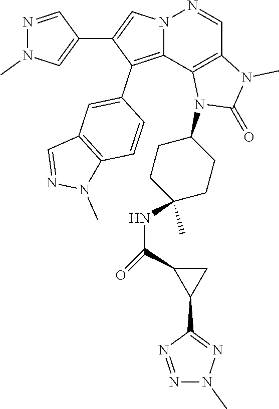 |
| I-299-2 | 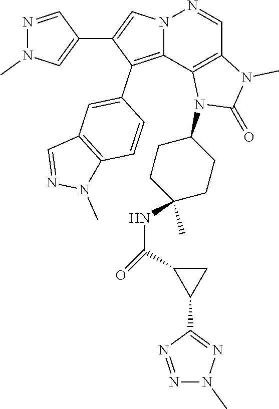 |
| I-299′ | 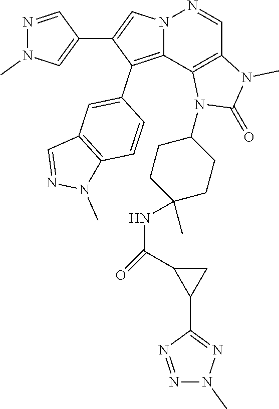 |
| I-300-1 | 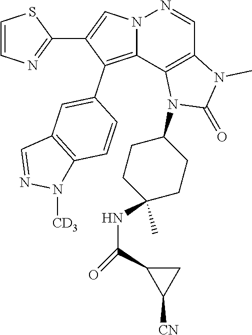 [SMILES: [2H]C([2H])([2H])n1ncc2cc(-c3c(-c4nccs4)cn4ncc5c(c34)n([C@H]3CC[C@@](C)(NC(=O)[C@H]4C[C@H]4C#N)CC3)c(=O)n5C)ccc21] |
| I-300-2 | 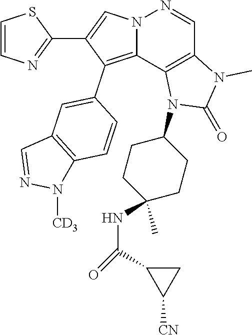 [SMILES: [2H]C([2H])([2H])n1ncc2cc(-c3c(-c4nccs4)cn4ncc5c(c34)n([C@H]3CC[C@@](C)(NC(=O)[C@@H]4C[C@@H]4C#N)CC3)c(=O)n5C)ccc21] |
| I-300′ | 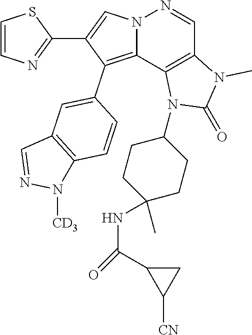 [SMILES: [2H]C([2H])([2H])n1ncc2cc(-c3c(-c4nccs4)cn4ncc5c(c34)n(C3CCC(C)(NC(=O)C4CC4C#N)CC3)c(=O)n5C)ccc21] |
| I-301-1 | 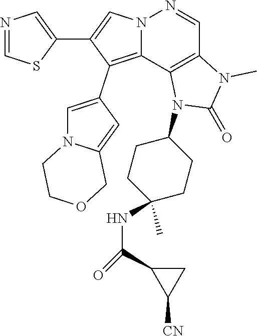 [SMILES: Cn1c(=O)n([C@H]2CC[C@@](C)(NC(=O)[C@H]3C[C@H]3C#N)CC2)c2c1cnn1cc(-c3cncs3)c(-c3cc4n(c3)CCOC4)c21] |
| I-301-2 | 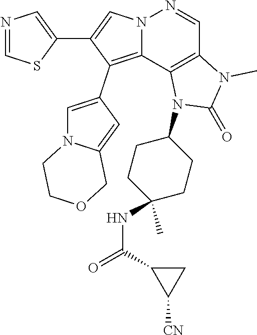 [SMILES: Cn1c(=O)n([C@H]2CC[C@@](C)(NC(=O)[C@@H]3C[C@@H]3C#N)CC2)c2c1cnn1cc(-c3cncs3)c(-c3cc4n(c3)CCOC4)c21] |
| I-301′ | 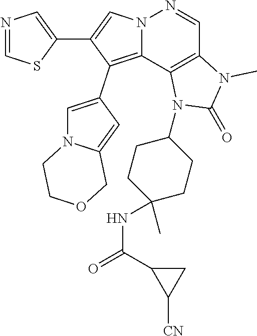 [SMILES: Cn1c(=O)n(C2CCC(C)(NC(=O)C3CC3C#N)CC2)c2c1cnn1cc(-c3cncs3)c(-c3cc4n(c3)CCOC4)c21] |
| I-302 | 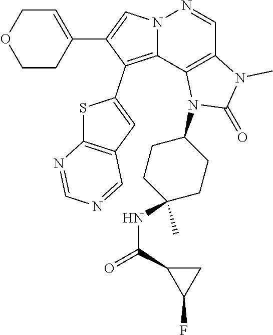 [SMILES: Cn1c(=O)n([C@H]2CC[C@@](C)(NC(=O)[C@H]3C[C@H]3F)CC2)c2c1cnn1cc(C3=CCOCC3)c(-c3cc4cncnc4s3)c21] |
| I-302′ | 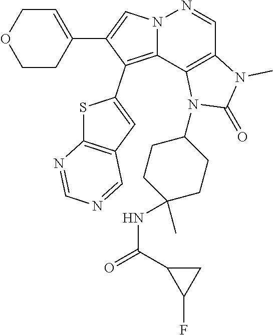 [SMILES: Cn1c(=O)n(C2CCC(C)(NC(=O)C3CC3F)CC2)c2c1cnn1cc(C3=CCOCC3)c(-c3cc4cncnc4s3)c21] |
| I-303-1 | 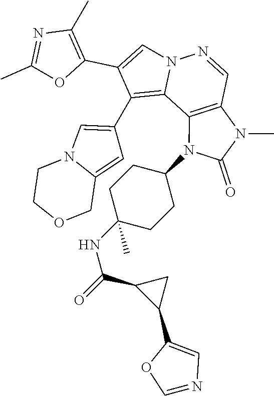 |
| I-303-2 | 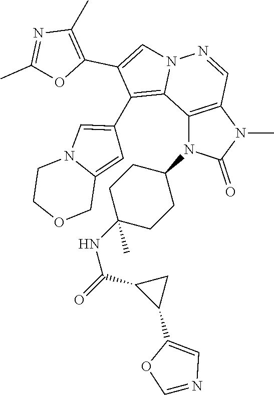 |
| I-303′ | 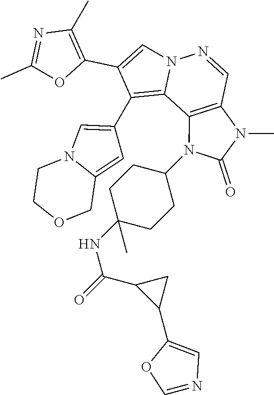 |
| I-304-1 | 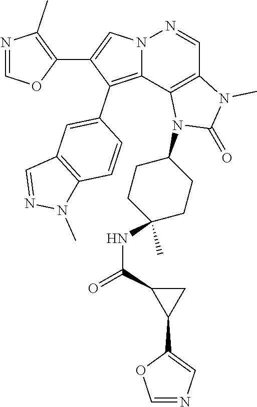 [SMILES: Cc1ncoc1-c1cn2ncc3c(c2c1-c1ccc2c(cnn2C)c1)n([C@H]1CC[C@@](C)(NC(=O)[C@H]2C[C@H]2c2cnco2)CC1)c(=O)n3C] |
| I-304-2 | 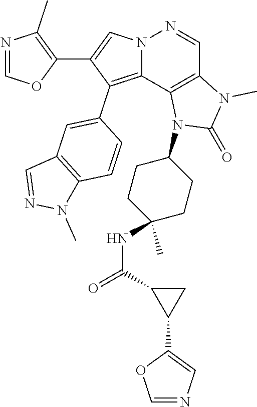 [SMILES: Cc1ncoc1-c1cn2ncc3c(c2c1-c1ccc2c(cnn2C)c1)n([C@H]1CC[C@@](C)(NC(=O)[C@@H]2C[C@@H]2c2cnco2)CC1)c(=O)n3C] |
| I-304′ | 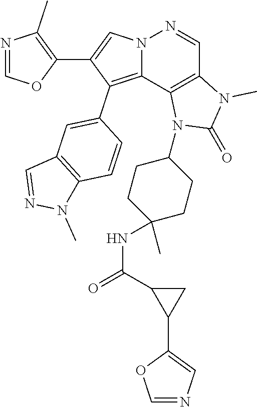 [SMILES: Cc1ncoc1-c1cn2ncc3c(c2c1-c1ccc2c(cnn2C)c1)n(C1CCC(C)(NC(=O)C2CC2c2cnco2)CC1)c(=O)n3C] |
| I-305 | 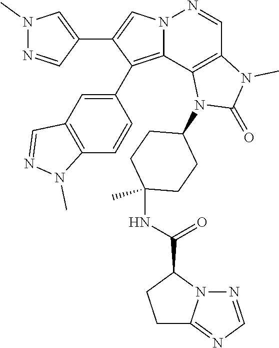 |
| I-305′ | 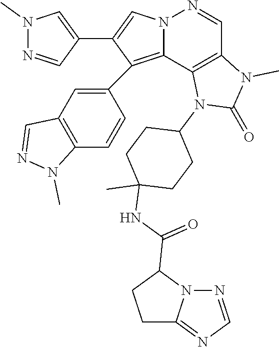 |
| I-306 | 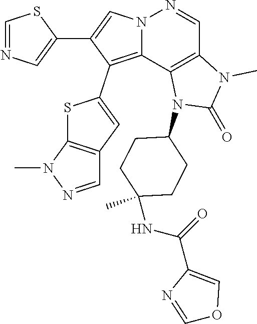 [SMILES: Cn1ncc2cc(-c3c(-c4cncs4)cn4ncc5c(c34)n([C@H]3CC[C@@](C)(NC(=O)c4cocn4)CC3)c(=O)n5C)sc21] |
| I-306′ | 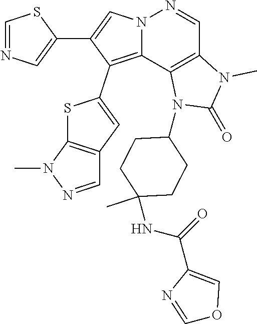 [SMILES: Cn1ncc2cc(-c3c(-c4cncs4)cn4ncc5c(c34)n(C3CCC(C)(NC(=O)c4cocn4)CC3)c(=O)n5C)sc21] |
| I-307 | 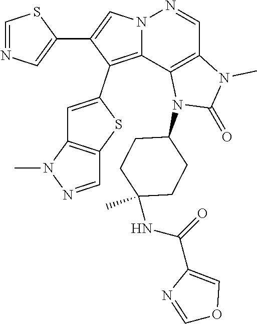 [SMILES: Cn1ncc2sc(-c3c(-c4cncs4)cn4ncc5c(c34)n([C@H]3CC[C@@](C)(NC(=O)c4cocn4)CC3)c(=O)n5C)cc21] |
| I-307′ | 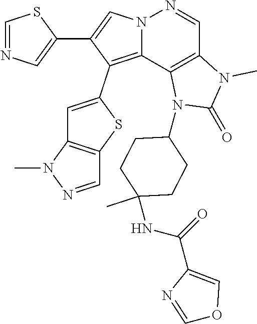 [SMILES: Cn1ncc2sc(-c3c(-c4cncs4)cn4ncc5c(c34)n(C3CCC(C)(NC(=O)c4cocn4)CC3)c(=O)n5C)cc21] |
| I-308 | 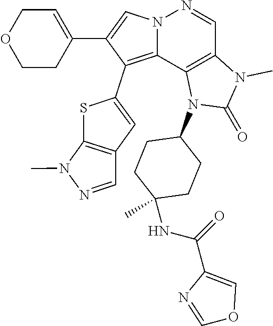 [SMILES: Cn1ncc2cc(-c3c(C4=CCOCC4)cn4ncc5c(c34)n([C@H]3CC[C@@](C)(NC(=O)c4cocn4)CC3)c(=O)n5C)sc21] |
| I-308′ | 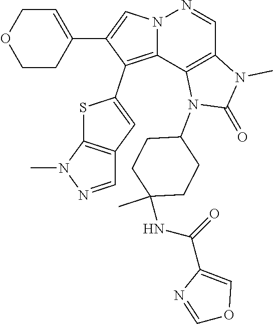 [SMILES: Cn1ncc2cc(-c3c(C4=CCOCC4)cn4ncc5c(c34)n(C3CCC(C)(NC(=O)c4cocn4)CC3)c(=O)n5C)sc21] |
| I-309 | 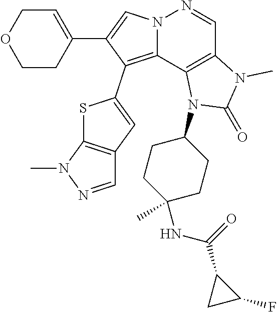 [SMILES: Cn1ncc2cc(-c3c(C4=CCOCC4)cn4ncc5c(c34)n([C@H]3CC[C@@](C)(NC(=O)[C@H]4C[C@H]4F)CC3)c(=O)n5C)sc21] |
| I-309′ | 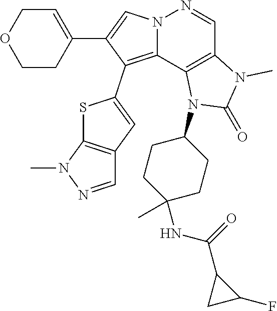 |
| I-310-1 | 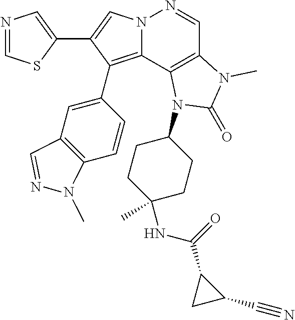 [SMILES: Cn1ncc2cc(-c3c(-c4cncs4)cn4ncc5c(c34)n([C@H]3CC[C@@](C)(NC(=O)[C@H]4C[C@H]4C#N)CC3)c(=O)n5C)ccc21] |
| I-310-2 | 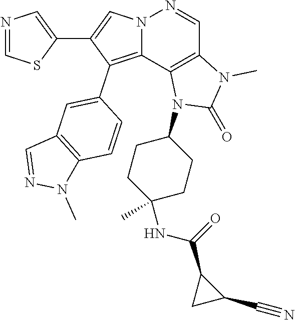 [SMILES: Cn1ncc2cc(-c3c(-c4cncs4)cn4ncc5c(c34)n([C@H]3CC[C@@](C)(NC(=O)[C@@H]4C[C@@H]4C#N)CC3)c(=O)n5C)ccc21] |
| I-310′ | 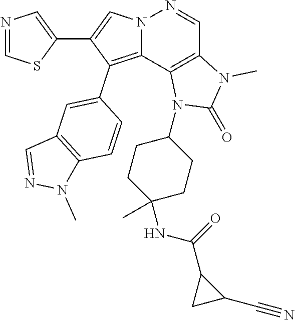 [SMILES: Cn1ncc2cc(-c3c(-c4cncs4)cn4ncc5c(c34)n(C3CCC(C)(NC(=O)C4CC4C#N)CC3)c(=O)n5C)ccc21] |
| I-311 | 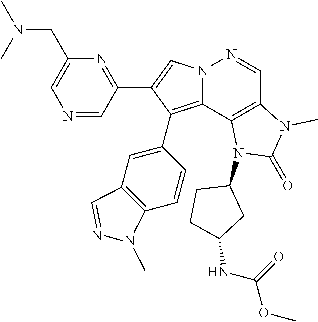 |
| I-311′ | 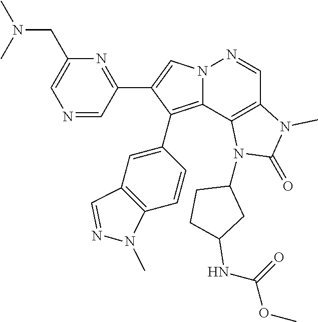 |
| I-312 | 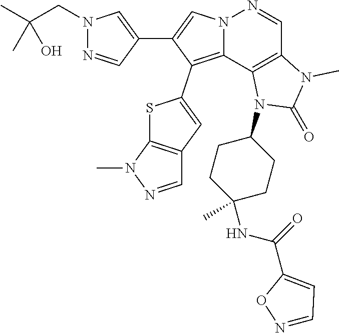 [SMILES: Cn1ncc2cc(-c3c(-c4cnn(CC(C)(C)O)c4)cn4ncc5c(c34)n([C@H]3CC[C@@](C)(NC(=O)c4ccno4)CC3)c(=O)n5C)sc21] |
| I-312′ | 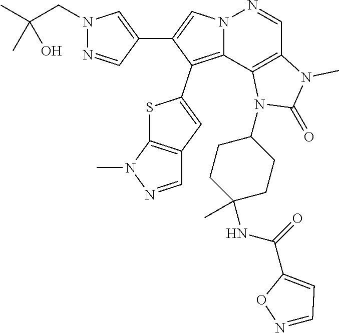 [SMILES: Cn1ncc2cc(-c3c(-c4cnn(CC(C)(C)O)c4)cn4ncc5c(c34)n(C3CCC(C)(NC(=O)c4ccno4)CC3)c(=O)n5C)sc21] |
| I-313 | 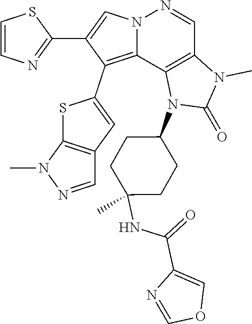 [SMILES: Cn1ncc2cc(-c3c(-c4nccs4)cn4ncc5c(c34)n([C@H]3CC[C@@](C)(NC(=O)c4cocn4)CC3)c(=O)n5C)sc21] |
| I-313′ | 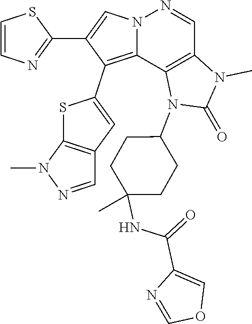 [SMILES: Cn1ncc2cc(-c3c(-c4nccs4)cn4ncc5c(c34)n(C3CCC(C)(NC(=O)c4cocn4)CC3)c(=O)n5C)sc21] |
| I-314 | 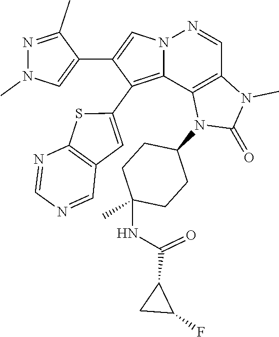 |
| I-314′ | 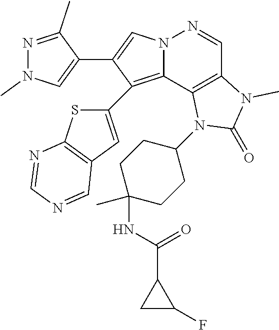 |
| I-315 | 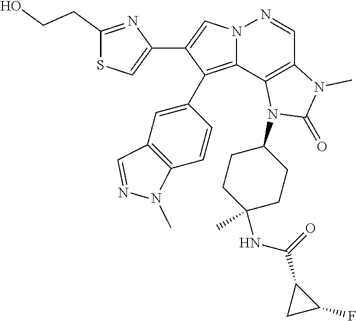 [SMILES: Cn1ncc2cc(-c3c(-c4csc(CCO)n4)cn4ncc5c(c34)n([C@H]3CC[C@@](C)(NC(=O)[C@H]4C[C@H]4F)CC3)c(=O)n5C)ccc21] |
| I-315′ | 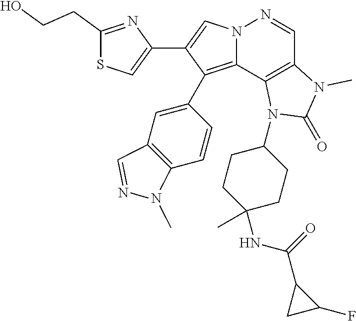 [SMILES: Cn1ncc2cc(-c3c(-c4csc(CCO)n4)cn4ncc5c(c34)n(C3CCC(C)(NC(=O)C4CC4F)CC3)c(=O)n5C)ccc21] |
| I-316 | 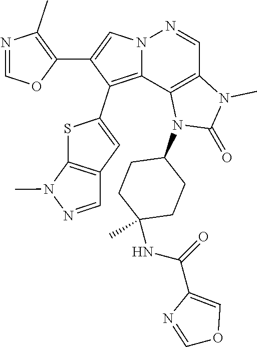 [SMILES: Cc1ncoc1-c1cn2ncc3c(c2c1-c1cc2cnn(C)c2s1)n([C@H]1CC[C@@](C)(NC(=O)c2cocn2)CC1)c(=O)n3C] |
| I-316′ | 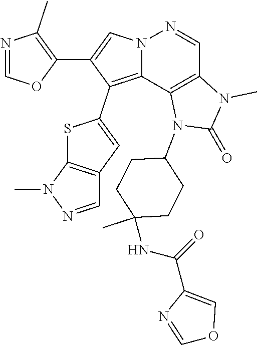 [SMILES: Cc1ncoc1-c1cn2ncc3c(c2c1-c1cc2cnn(C)c2s1)n(C1CCC(C)(NC(=O)c2cocn2)CC1)c(=O)n3C] |
| I-317 | 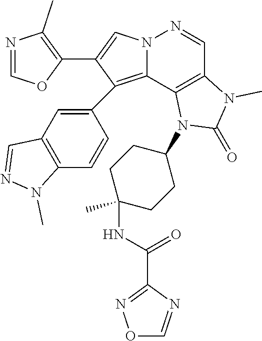 [SMILES: Cc1ncoc1-c1cn2ncc3c(c2c1-c1ccc2c(cnn2C)c1)n([C@H]1CC[C@@](C)(NC(=O)c2ncon2)CC1)c(=O)n3C] |
| I-317′ | 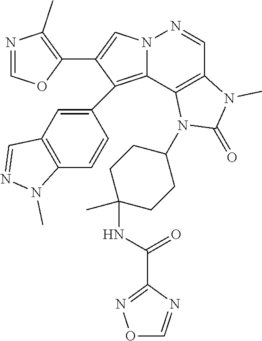 [SMILES: Cc1ncoc1-c1cn2ncc3c(c2c1-c1ccc2c(cnn2C)c1)n(C1CCC(C)(NC(=O)c2ncon2)CC1)c(=O)n3C] |
| I-318 | 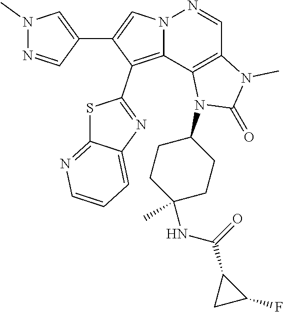 [SMILES: Cn1cc(-c2cn3ncc4c(c3c2-c2nc3cccnc3s2)n([C@H]2CC[C@@](C)(NC(=O)[C@H]3C[C@H]3F)CC2)c(=O)n4C)cn1] |
| I-318′ | 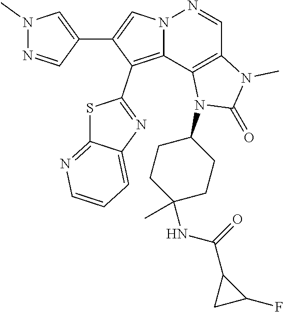 |
| I-319 | 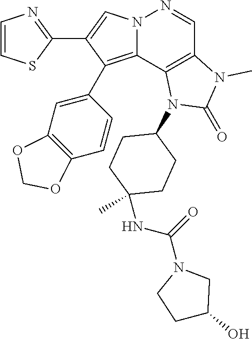 [SMILES: Cn1c(=O)n([C@H]2CC[C@@](C)(NC(=O)N3CC[C@@H](O)C3)CC2)c2c1cnn1cc(-c3nccs3)c(-c3ccc4c(c3)OCO4)c21] |
| I-319′ | 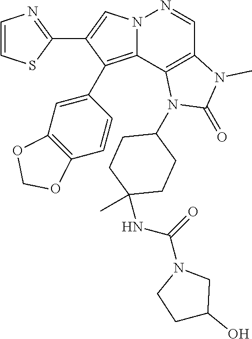 [SMILES: Cn1c(=O)n(C2CCC(C)(NC(=O)N3CCC(O)C3)CC2)c2c1cnn1cc(-c3nccs3)c(-c3ccc4c(c3)OCO4)c21] |
| I-320 | 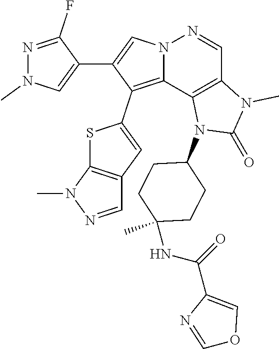 |
| I-320′ | 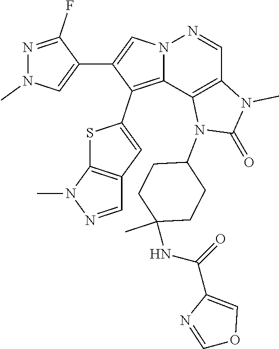 |
| I-321-1 | 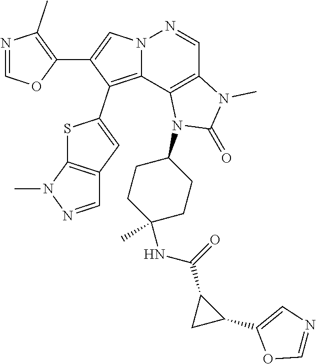 [SMILES: Cc1ncoc1-c1cn2ncc3c(c2c1-c1cc2cnn(C)c2s1)n([C@H]1CC[C@@](C)(NC(=O)[C@H]2C[C@H]2c2cnco2)CC1)c(=O)n3C] |
| I-321-2 | 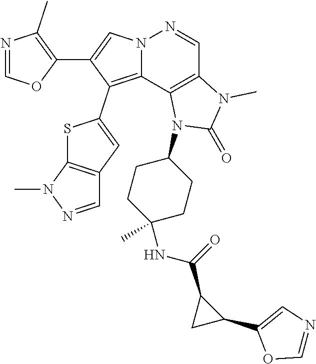 [SMILES: Cc1ncoc1-c1cn2ncc3c(c2c1-c1cc2cnn(C)c2s1)n([C@H]1CC[C@@](C)(NC(=O)[C@@H]2C[C@@H]2c2cnco2)CC1)c(=O)n3C] |
| I-321′ | 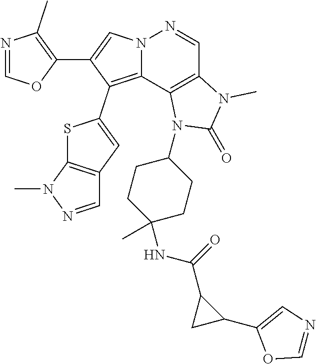 [SMILES: Cc1ncoc1-c1cn2ncc3c(c2c1-c1cc2cnn(C)c2s1)n(C1CCC(C)(NC(=O)C2CC2c2cnco2)CC1)c(=O)n3C] |
| I-322 | 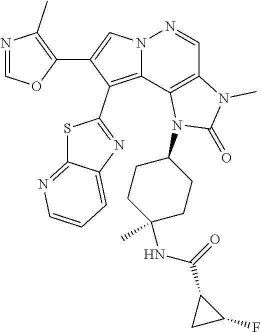 [SMILES: Cc1ncoc1-c1cn2ncc3c(c2c1-c1nc2cccnc2s1)n([C@H]1CC[C@@](C)(NC(=O)[C@H]2C[C@H]2F)CC1)c(=O)n3C] |
| I-322′ | 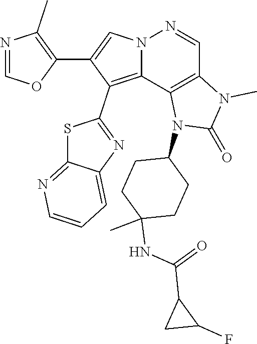 |
| I-323 | 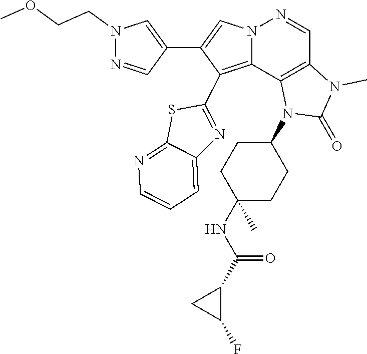 |
| I-323′ | 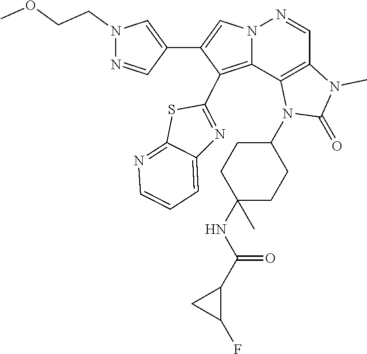 |
| I-324 | 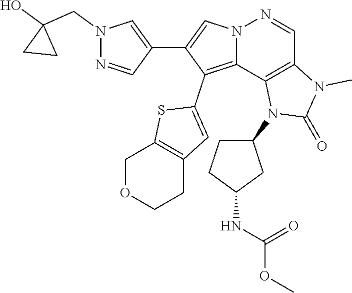 [SMILES: COC(=O)N[C@@H]1CC[C@@H](n2c(=O)n(C)c3cnn4cc(-c5cnn(CC6(O)CC6)c5)c(-c5cc6c(s5)COCC6)c4c32)C1] |
| I-324′ | 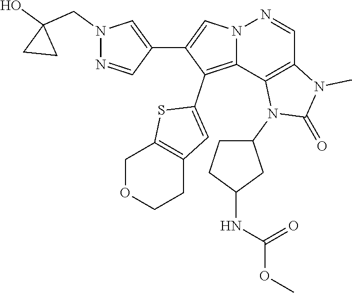 [SMILES: COC(=O)NC1CCC(n2c(=O)n(C)c3cnn4cc(-c5cnn(CC6(O)CC6)c5)c(-c5cc6c(s5)COCC6)c4c32)C1] |
| I-325 | 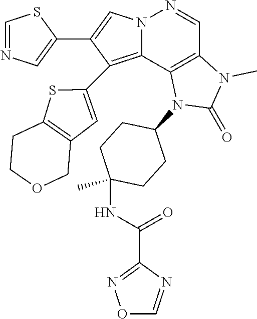 [SMILES: Cn1c(=O)n([C@H]2CC[C@@](C)(NC(=O)c3ncon3)CC2)c2c1cnn1cc(-c3cncs3)c(-c3cc4c(s3)CCOC4)c21] |
| I-325′ | 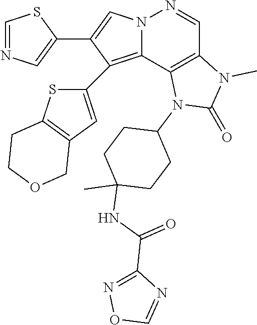 [SMILES: Cn1c(=O)n(C2CCC(C)(NC(=O)c3ncon3)CC2)c2c1cnn1cc(-c3cncs3)c(-c3cc4c(s3)CCOC4)c21] |
| I-326-1 | 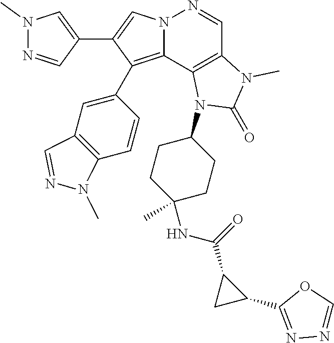 [SMILES: Cn1cc(-c2cn3ncc4c(c3c2-c2ccc3c(cnn3C)c2)n([C@H]2CC[C@@](C)(NC(=O)[C@H]3C[C@H]3c3nnco3)CC2)c(=O)n4C)cn1] |
| I-326-2 | 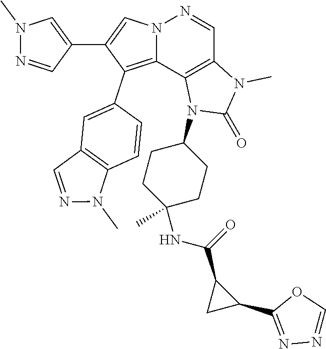 [SMILES: Cn1cc(-c2cn3ncc4c(c3c2-c2ccc3c(cnn3C)c2)n([C@H]2CC[C@@](C)(NC(=O)[C@@H]3C[C@@H]3c3nnco3)CC2)c(=O)n4C)cn1] |
| I-326′ | 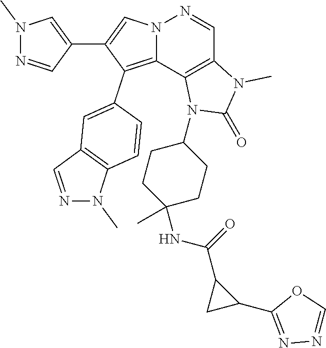 [SMILES: Cn1cc(-c2cn3ncc4c(c3c2-c2ccc3c(cnn3C)c2)n(C2CCC(C)(NC(=O)C3CC3c3nnco3)CC2)c(=O)n4C)cn1] |
| I-327 | 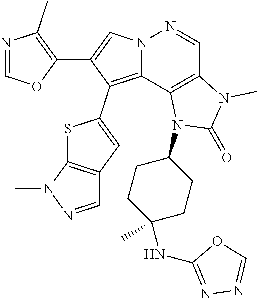 [SMILES: Cc1ncoc1-c1cn2ncc3c(c2c1-c1cc2cnn(C)c2s1)n([C@H]1CC[C@@](C)(Nc2nnco2)CC1)c(=O)n3C] |
| I-327′ | 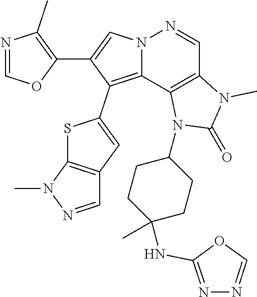 [SMILES: Cc1ncoc1-c1cn2ncc3c(c2c1-c1cc2cnn(C)c2s1)n(C1CCC(C)(Nc2nnco2)CC1)c(=O)n3C] |
| I-328 | 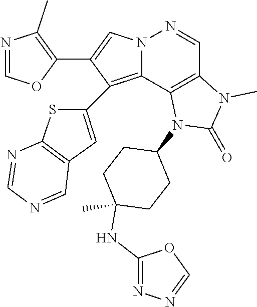 [SMILES: Cc1ncoc1-c1cn2ncc3c(c2c1-c1cc2cncnc2s1)n([C@H]1CC[C@@](C)(Nc2nnco2)CC1)c(=O)n3C] |
| I-328′ | 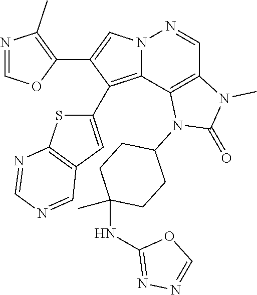 [SMILES: Cc1ncoc1-c1cn2ncc3c(c2c1-c1cc2cncnc2s1)n(C1CCC(C)(Nc2nnco2)CC1)c(=O)n3C] |
| I-329-1 | 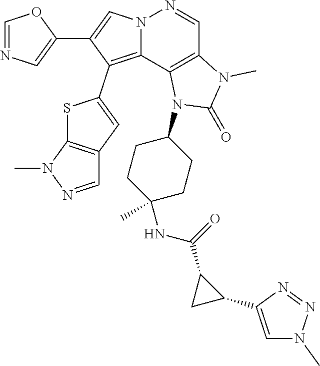 [SMILES: Cn1cc([C@@H]2C[C@@H]2C(=O)N[C@]2(C)CC[C@H](n3c(=O)n(C)c4cnn5cc(-c6cnco6)c(-c6cc7cnn(C)c7s6)c5c43)CC2)nn1] |
| I-329-2 | 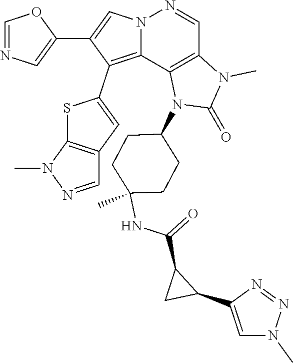 [SMILES: Cn1cc([C@H]2C[C@H]2C(=O)N[C@]2(C)CC[C@H](n3c(=O)n(C)c4cnn5cc(-c6cnco6)c(-c6cc7cnn(C)c7s6)c5c43)CC2)nn1] |
| I-329′ | 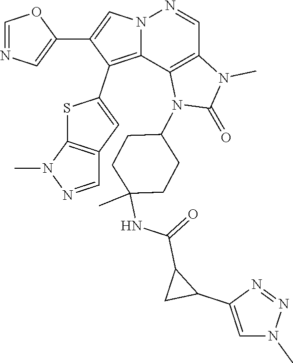 [SMILES: Cn1cc(C2CC2C(=O)NC2(C)CCC(n3c(=O)n(C)c4cnn5cc(-c6cnco6)c(-c6cc7cnn(C)c7s6)c5c43)CC2)nn1] |
| I-330-1 | 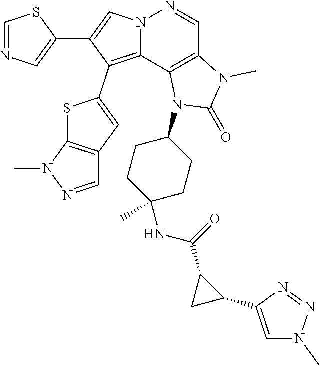 [SMILES: Cn1cc([C@@H]2C[C@@H]2C(=O)N[C@]2(C)CC[C@H](n3c(=O)n(C)c4cnn5cc(-c6cncs6)c(-c6cc7cnn(C)c7s6)c5c43)CC2)nn1] |
| I-330-2 | 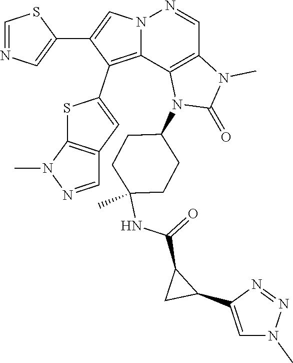 [SMILES: Cn1cc([C@H]2C[C@H]2C(=O)N[C@]2(C)CC[C@H](n3c(=O)n(C)c4cnn5cc(-c6cncs6)c(-c6cc7cnn(C)c7s6)c5c43)CC2)nn1] |
| I-330′ | 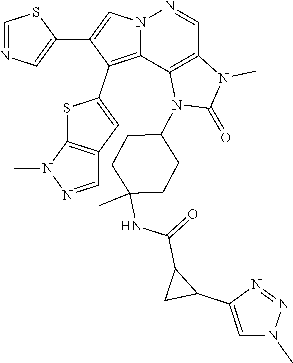 [SMILES: Cn1cc(C2CC2C(=O)NC2(C)CCC(n3c(=O)n(C)c4cnn5cc(-c6cncs6)c(-c6cc7cnn(C)c7s6)c5c43)CC2)nn1] |
| I-331 | 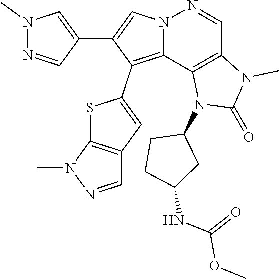 |
| I-331′ | 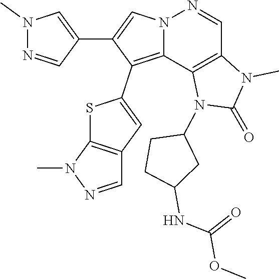 |
| I-332 | 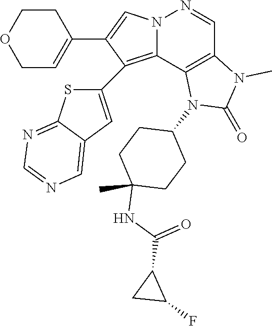 [SMILES: Cn1c(=O)n([C@H]2CC[C@@](C)(NC(=O)[C@H]3C[C@H]3F)CC2)c2c1cnn1cc(C3=CCOCC3)c(-c3cc4cncnc4s3)c21] |
| I-332′ | 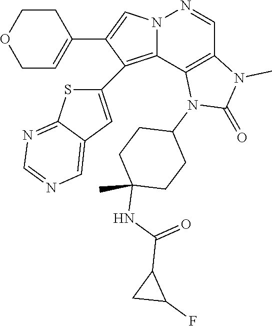 |
| I-333 | 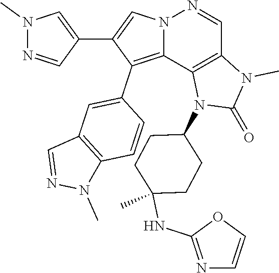 |
| I-333′ | 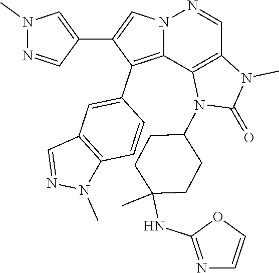 |
| I-334 | 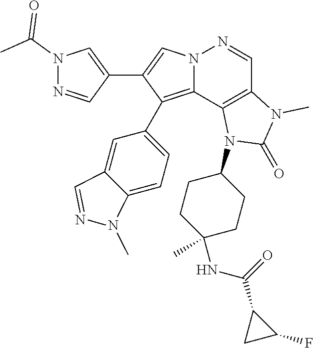 [SMILES: CC(=O)n1cc(-c2cn3ncc4c(c3c2-c2ccc3c(cnn3C)c2)n([C@H]2CC[C@@](C)(NC(=O)[C@H]3C[C@H]3F)CC2)c(=O)n4C)cn1] |
| I-334′ | 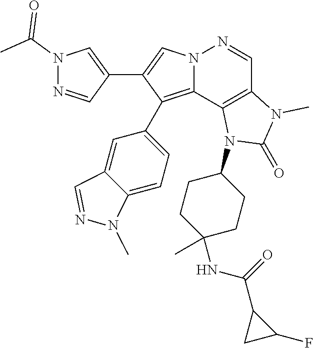 |
| I-335 | 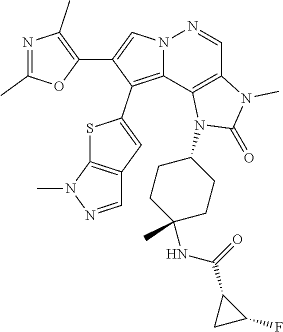 [SMILES: Cc1nc(C)c(-c2cn3ncc4c(c3c2-c2cc3cnn(C)c3s2)n([C@H]2CC[C@@](C)(NC(=O)[C@H]3C[C@H]3F)CC2)c(=O)n4C)o1] |
| I-335′ | 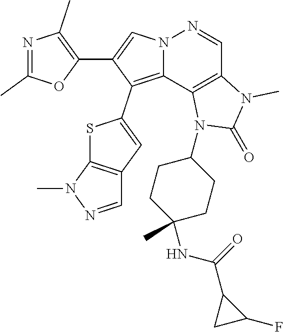 |
| I-336 | 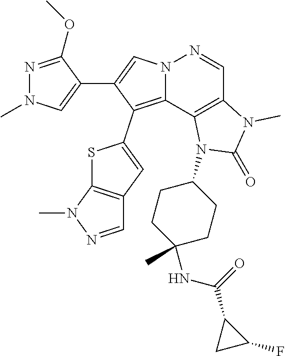 [SMILES: COc1nn(C)cc1-c1cn2ncc3c(c2c1-c1cc2cnn(C)c2s1)n([C@H]1CC[C@@](C)(NC(=O)[C@H]2C[C@H]2F)CC1)c(=O)n3C] |
| I-336′ | 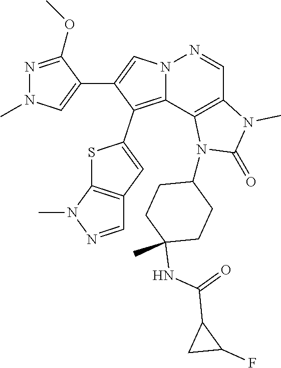 |
| I-337 | 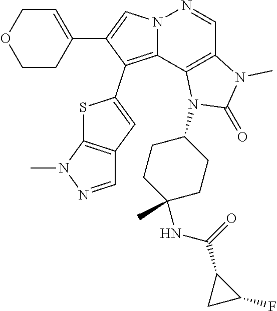 [SMILES: Cn1ncc2cc(-c3c(C4=CCOCC4)cn4ncc5c(c34)n([C@H]3CC[C@@](C)(NC(=O)[C@H]4C[C@H]4F)CC3)c(=O)n5C)sc21] |
| I-337′ | 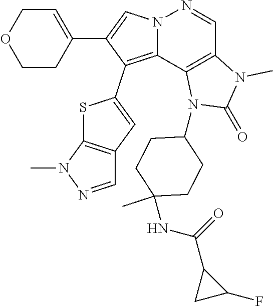 [SMILES: Cn1ncc2cc(-c3c(C4=CCOCC4)cn4ncc5c(c34)n(C3CCC(C)(NC(=O)C4CC4F)CC3)c(=O)n5C)sc21] |
| I-338 | 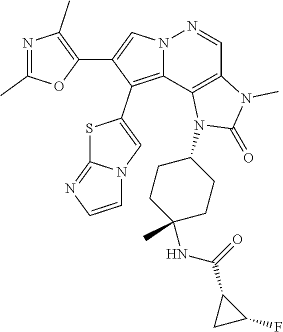 [SMILES: Cc1nc(C)c(-c2cn3ncc4c(c3c2-c2cn3ccnc3s2)n([C@H]2CC[C@@](C)(NC(=O)[C@H]3C[C@H]3F)CC2)c(=O)n4C)o1] |
| I-338′ | 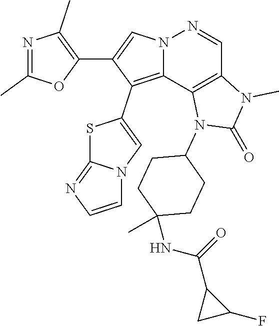 |
| I-339 | 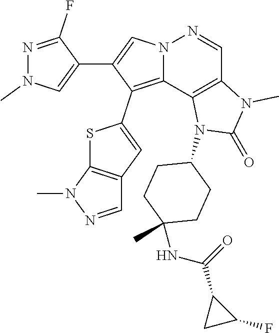 |
| I-339′ | 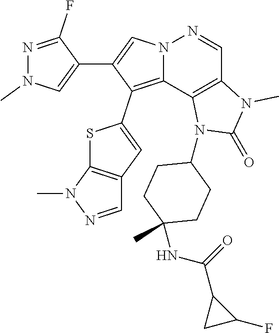 |
| I-340 | 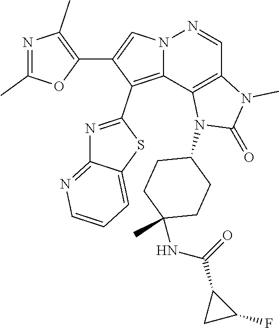 |
| I-340′ | 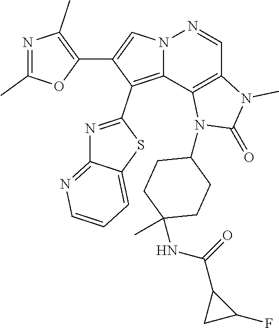 |
| I-341-1 | 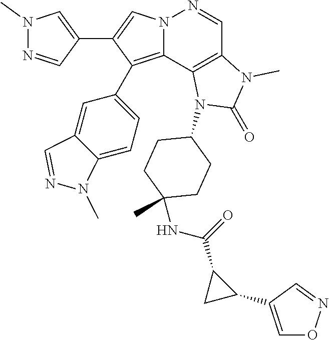 [SMILES: Cn1cc(-c2cn3ncc4c(c3c2-c2ccc3c(cnn3C)c2)n([C@H]2CC[C@@](C)(NC(=O)[C@H]3C[C@H]3c3cnoc3)CC2)c(=O)n4C)cn1] |
| I-341-2 | 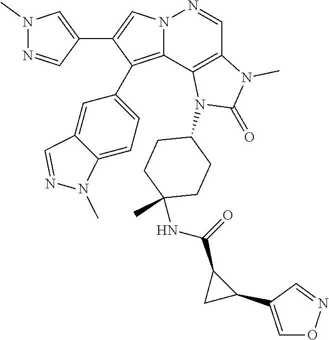 [SMILES: Cn1cc(-c2cn3ncc4c(c3c2-c2ccc3c(cnn3C)c2)n([C@H]2CC[C@@](C)(NC(=O)[C@@H]3C[C@@H]3c3cnoc3)CC2)c(=O)n4C)cn1] |
| I-341′ | 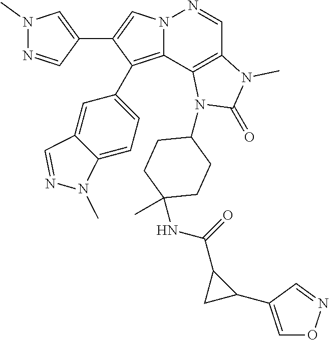 [SMILES: Cn1cc(-c2cn3ncc4c(c3c2-c2ccc3c(cnn3C)c2)n(C2CCC(C)(NC(=O)C3CC3c3cnoc3)CC2)c(=O)n4C)cn1] |
| I-342-1 | 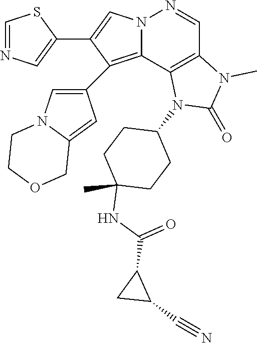 [SMILES: Cn1c(=O)n([C@H]2CC[C@@](C)(NC(=O)[C@H]3C[C@H]3C#N)CC2)c2c1cnn1cc(-c3cncs3)c(-c3cc4n(c3)CCOC4)c21] |
| I-342-2 | 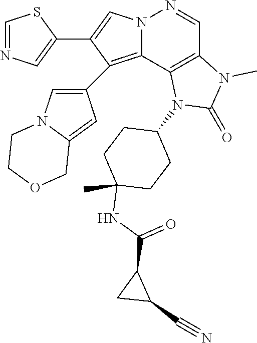 [SMILES: Cn1c(=O)n([C@H]2CC[C@@](C)(NC(=O)[C@@H]3C[C@@H]3C#N)CC2)c2c1cnn1cc(-c3cncs3)c(-c3cc4n(c3)CCOC4)c21] |
| I-342′ | 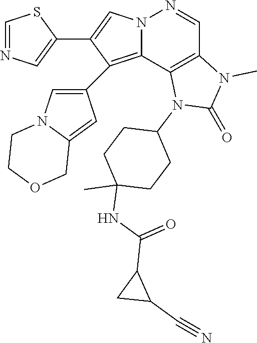 [SMILES: Cn1c(=O)n(C2CCC(C)(NC(=O)C3CC3C#N)CC2)c2c1cnn1cc(-c3cncs3)c(-c3cc4n(c3)CCOC4)c21] |
| I-343-1 | 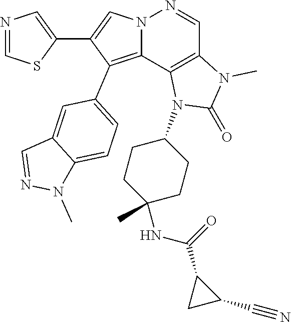 [SMILES: Cn1ncc2cc(-c3c(-c4cncs4)cn4ncc5c(c34)n([C@H]3CC[C@@](C)(NC(=O)[C@H]4C[C@H]4C#N)CC3)c(=O)n5C)ccc21] |
| I-343-2 | 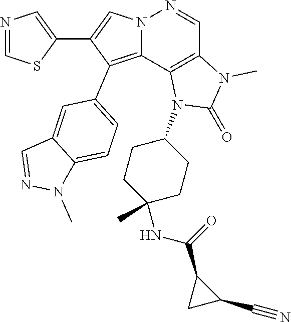 [SMILES: Cn1ncc2cc(-c3c(-c4cncs4)cn4ncc5c(c34)n([C@H]3CC[C@@](C)(NC(=O)[C@@H]4C[C@@H]4C#N)CC3)c(=O)n5C)ccc21] |
| I-343′ | 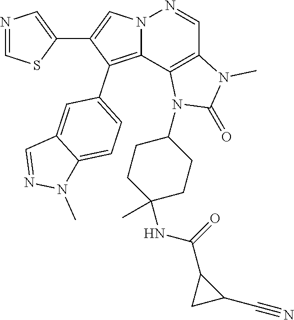 [SMILES: Cn1ncc2cc(-c3c(-c4cncs4)cn4ncc5c(c34)n(C3CCC(C)(NC(=O)C4CC4C#N)CC3)c(=O)n5C)ccc21] |
| I-344 | 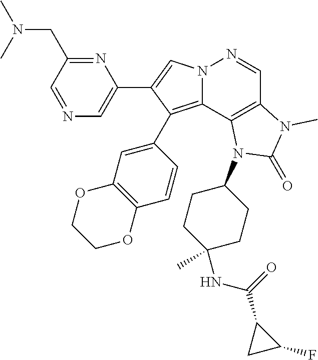 |
| I-344′ | 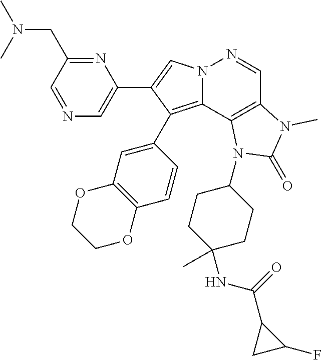 [SMILES: CN(C)Cc1cncc(-c2cn3ncc4c(c3c2-c2ccc3c(c2)OCCO3)n(C2CCC(C)(NC(=O)C3CC3F)CC2)c(=O)n4C)n1] |
| I-345 | 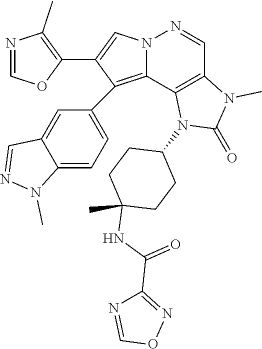 [SMILES: Cc1ncoc1-c1cn2ncc3c(c2c1-c1ccc2c(cnn2C)c1)n([C@H]1CC[C@@](C)(NC(=O)c2ncon2)CC1)c(=O)n3C] |
| I-345′ | 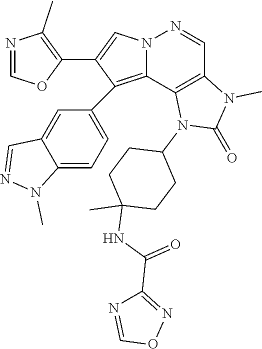 [SMILES: Cc1ncoc1-c1cn2ncc3c(c2c1-c1ccc2c(cnn2C)c1)n(C1CCC(C)(NC(=O)c2ncon2)CC1)c(=O)n3C] |
| I-346 | 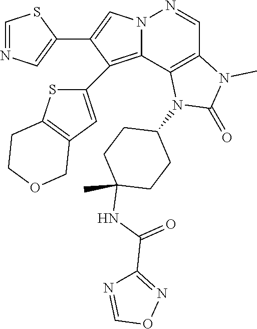 [SMILES: Cn1c(=O)n([C@H]2CC[C@@](C)(NC(=O)c3ncon3)CC2)c2c1cnn1cc(-c3cncs3)c(-c3cc4c(s3)CCOC4)c21] |
| I-346′ | 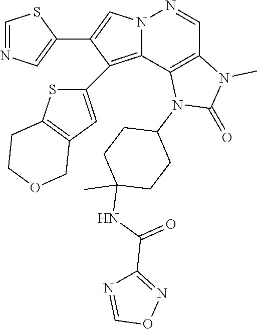 [SMILES: Cn1c(=O)n(C2CCC(C)(NC(=O)c3ncon3)CC2)c2c1cnn1cc(-c3cncs3)c(-c3cc4c(s3)CCOC4)c21] |
| I-347-1 | 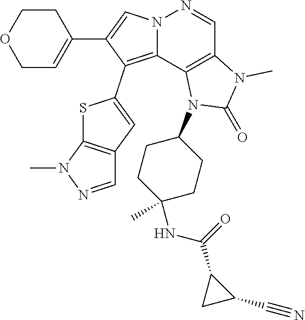 [SMILES: Cn1ncc2cc(-c3c(C4=CCOCC4)cn4ncc5c(c34)n([C@H]3CC[C@@](C)(NC(=O)[C@H]4C[C@H]4C#N)CC3)c(=O)n5C)sc21] |
| I-347-2 | 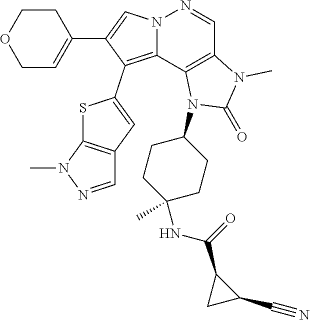 [SMILES: Cn1ncc2cc(-c3c(C4=CCOCC4)cn4ncc5c(c34)n([C@H]3CC[C@@](C)(NC(=O)[C@@H]4C[C@@H]4C#N)CC3)c(=O)n5C)sc21] |
| I-347′ | 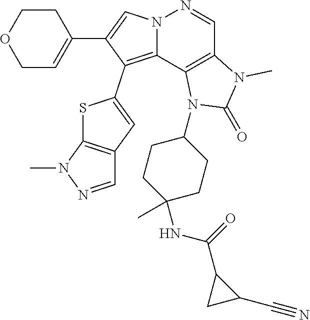 [SMILES: Cn1ncc2cc(-c3c(C4=CCOCC4)cn4ncc5c(c34)n(C3CCC(C)(NC(=O)C4CC4C#N)CC3)c(=O)n5C)sc21] |
| I-348-1 | 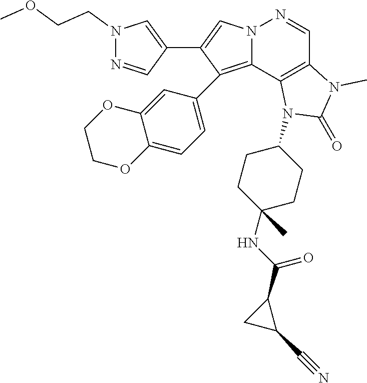 |
| I-348-2 | 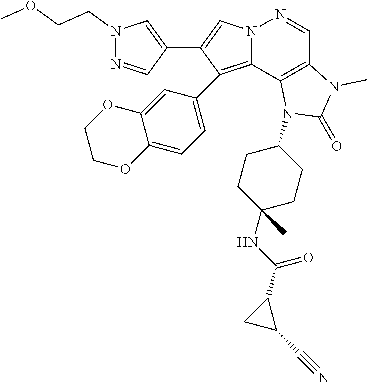 |
| I-348′ | 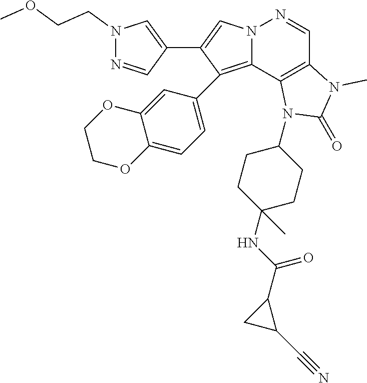 |
| I-349-1 | 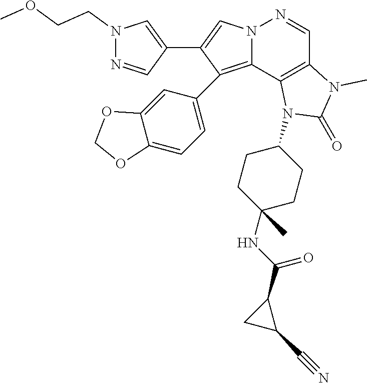 |
| I-349-2 | 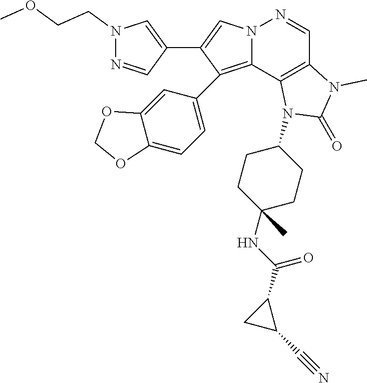 |
| I-349′ | 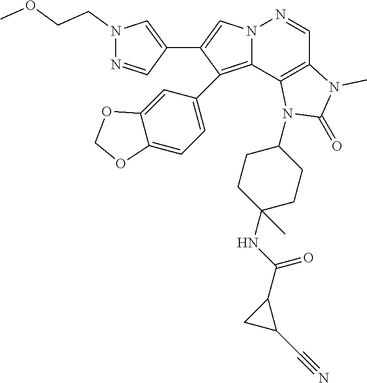 |
| I-350 | 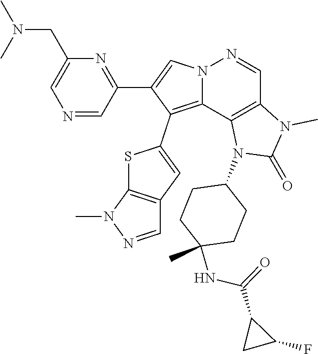 |
| I-350′ | 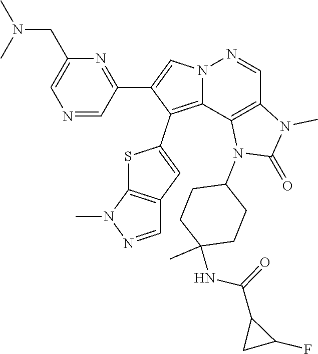 |
| I-351-1 | 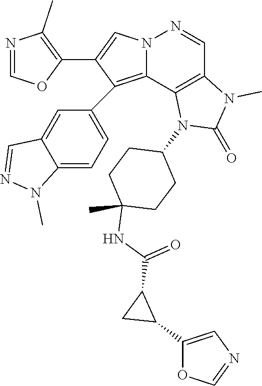 [SMILES: Cc1ncoc1-c1cn2ncc3c(c2c1-c1ccc2c(cnn2C)c1)n([C@H]1CC[C@@](C)(NC(=O)[C@H]2C[C@H]2c2cnco2)CC1)c(=O)n3C] |
| I-351-2 | 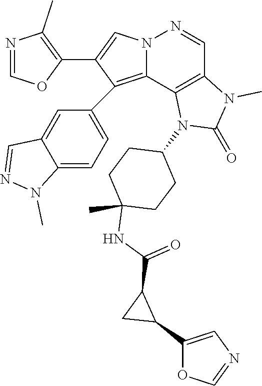 [SMILES: Cc1ncoc1-c1cn2ncc3c(c2c1-c1ccc2c(cnn2C)c1)n([C@H]1CC[C@@](C)(NC(=O)[C@@H]2C[C@@H]2c2cnco2)CC1)c(=O)n3C] |
| I-351′ | 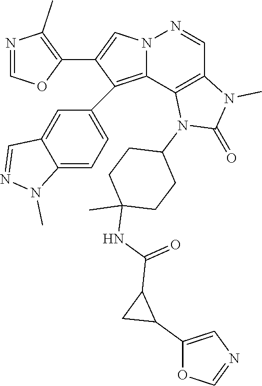 [SMILES: Cc1ncoc1-c1cn2ncc3c(c2c1-c1ccc2c(cnn2C)c1)n(C1CCC(C)(NC(=O)C2CC2c2cnco2)CC1)c(=O)n3C] |
| I-352-1 | 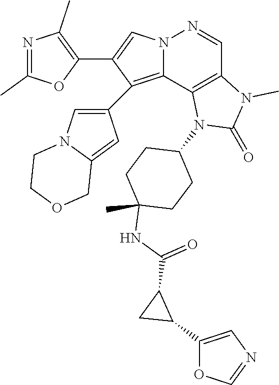 |
| I-352-2 | 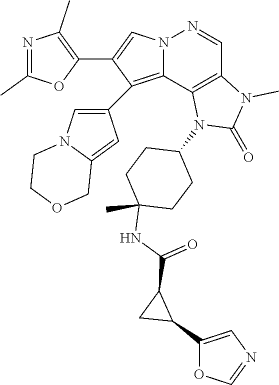 |
| I-352′ | 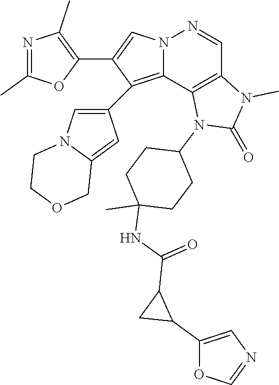 |
| I-353 | 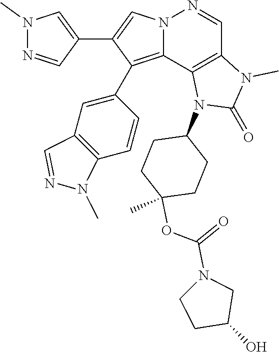 |
| I-353′ | 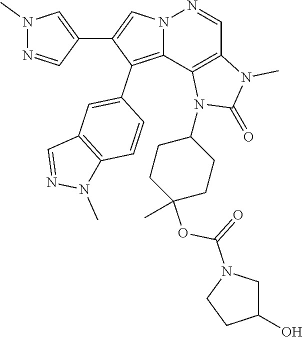 [SMILES: Cn1cc(-c2cn3ncc4c(c3c2-c2ccc3c(cnn3C)c2)n(C2CCC(C)(OC(=O)N3CCC(O)C3)CC2)c(=O)n4C)cn1] |
| I-354 | 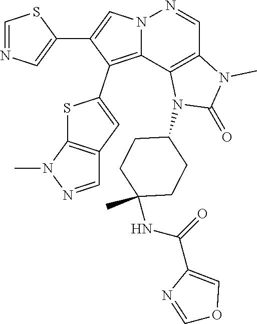 [SMILES: Cn1ncc2cc(-c3c(-c4cncs4)cn4ncc5c(c34)n([C@H]3CC[C@@](C)(NC(=O)c4cocn4)CC3)c(=O)n5C)sc21] |
| I-354′ | 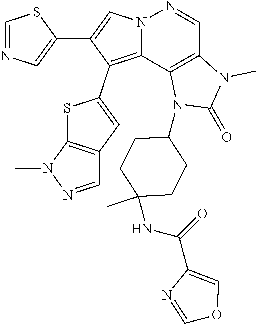 [SMILES: Cn1ncc2cc(-c3c(-c4cncs4)cn4ncc5c(c34)n(C3CCC(C)(NC(=O)c4cocn4)CC3)c(=O)n5C)sc21] |
| I-355 | 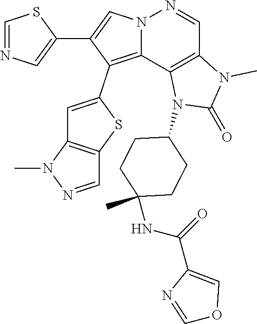 [SMILES: Cn1ncc2sc(-c3c(-c4cncs4)cn4ncc5c(c34)n([C@H]3CC[C@@](C)(NC(=O)c4cocn4)CC3)c(=O)n5C)cc21] |
| I-355′ | 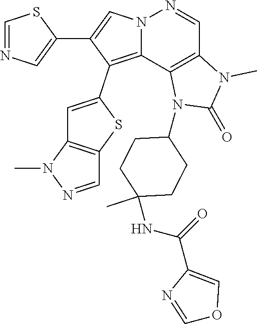 [SMILES: Cn1ncc2sc(-c3c(-c4cncs4)cn4ncc5c(c34)n(C3CCC(C)(NC(=O)c4cocn4)CC3)c(=O)n5C)cc21] |
| I-356 | 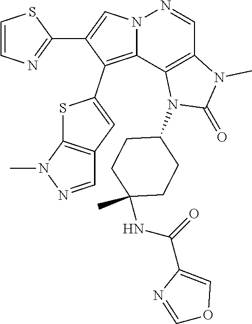 [SMILES: Cn1ncc2cc(-c3c(-c4nccs4)cn4ncc5c(c34)n([C@H]3CC[C@@](C)(NC(=O)c4cocn4)CC3)c(=O)n5C)sc21] |
| I-356′ | 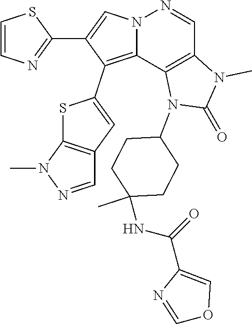 [SMILES: Cn1ncc2cc(-c3c(-c4nccs4)cn4ncc5c(c34)n(C3CCC(C)(NC(=O)c4cocn4)CC3)c(=O)n5C)sc21] |
| I-357 | 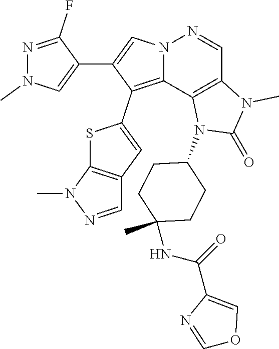 |
| I-357′ | 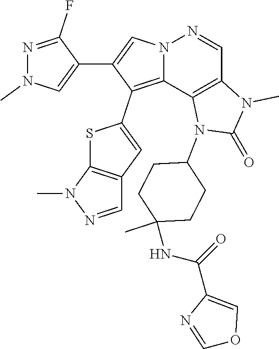 |
| I-358 | 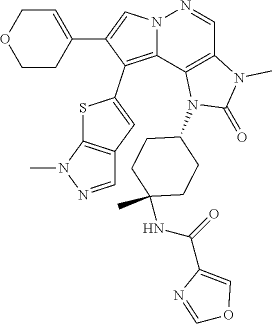 [SMILES: Cn1ncc2cc(-c3c(C4=CCOCC4)cn4ncc5c(c34)n([C@H]3CC[C@@](C)(NC(=O)c4cocn4)CC3)c(=O)n5C)sc21] |
| I-358′ | 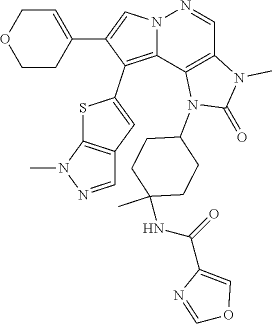 [SMILES: Cn1ncc2cc(-c3c(C4=CCOCC4)cn4ncc5c(c34)n(C3CCC(C)(NC(=O)c4cocn4)CC3)c(=O)n5C)sc21] |
| I-359 | 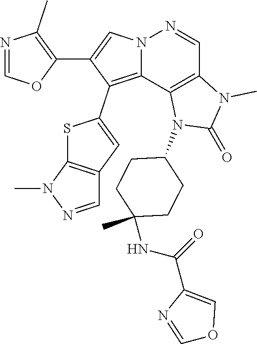 [SMILES: Cc1ncoc1-c1cn2ncc3c(c2c1-c1cc2cnn(C)c2s1)n([C@H]1CC[C@@](C)(NC(=O)c2cocn2)CC1)c(=O)n3C] |
| I-359′ | 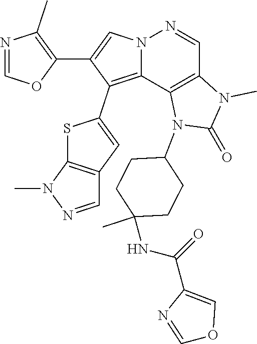 [SMILES: Cc1ncoc1-c1cn2ncc3c(c2c1-c1cc2cnn(C)c2s1)n(C1CCC(C)(NC(=O)c2cocn2)CC1)c(=O)n3C] |
| I-360 | 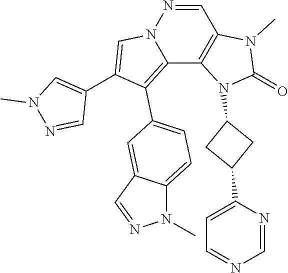 [SMILES: Cn1cc(-c2cn3ncc4c(c3c2-c2ccc3c(cnn3C)c2)n([C@H]2C[C@@H](c3ccncn3)C2)c(=O)n4C)cn1] |
| I-360′ | 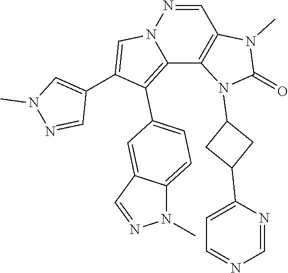 [SMILES: Cn1cc(-c2cn3ncc4c(c3c2-c2ccc3c(cnn3C)c2)n(C2CC(c3ccncn3)C2)c(=O)n4C)cn1] |
| I-361 | 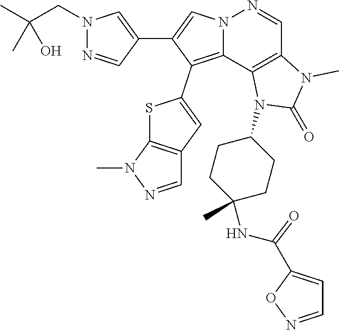 [SMILES: Cn1ncc2cc(-c3c(-c4cnn(CC(C)(C)O)c4)cn4ncc5c(c34)n([C@H]3CC[C@@](C)(NC(=O)c4ccno4)CC3)c(=O)n5C)sc21] |
| I-361′ | 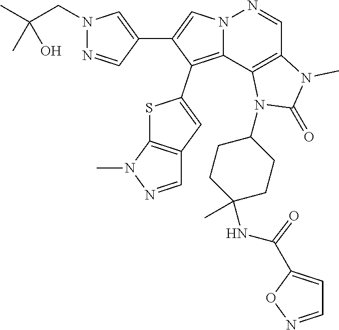 [SMILES: Cn1ncc2cc(-c3c(-c4cnn(CC(C)(C)O)c4)cn4ncc5c(c34)n(C3CCC(C)(NC(=O)c4ccno4)CC3)c(=O)n5C)sc21] |
| I-362 | 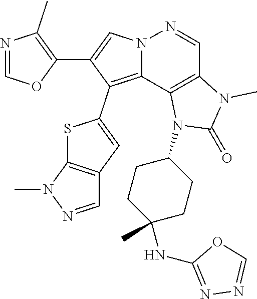 [SMILES: Cc1ncoc1-c1cn2ncc3c(c2c1-c1cc2cnn(C)c2s1)n([C@H]1CC[C@@](C)(Nc2nnco2)CC1)c(=O)n3C] |
| I-362′ | 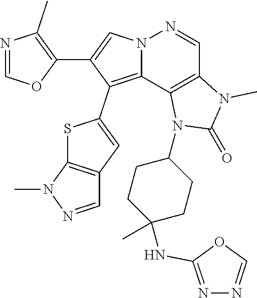 [SMILES: Cc1ncoc1-c1cn2ncc3c(c2c1-c1cc2cnn(C)c2s1)n(C1CCC(C)(Nc2nnco2)CC1)c(=O)n3C] |
| I-363 | 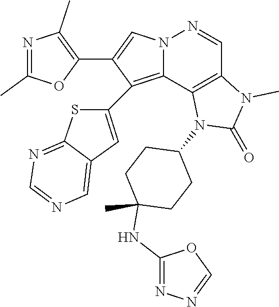 |
| I-363′ | 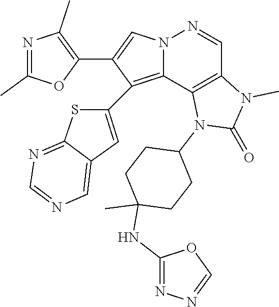 |
| I-364 | 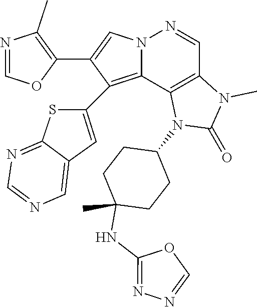 [SMILES: Cc1ncoc1-c1cn2ncc3c(c2c1-c1cc2cncnc2s1)n([C@H]1CC[C@@](C)(Nc2nnco2)CC1)c(=O)n3C] |
| I-364′ | 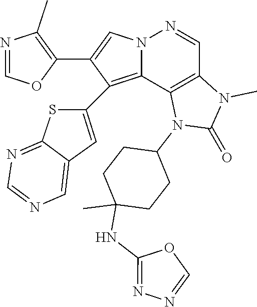 [SMILES: Cc1ncoc1-c1cn2ncc3c(c2c1-c1cc2cncnc2s1)n(C1CCC(C)(Nc2nnco2)CC1)c(=O)n3C] |
| I-365 | 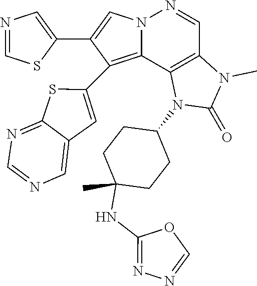 [SMILES: Cn1c(=O)n([C@H]2CC[C@@](C)(Nc3nnco3)CC2)c2c1cnn1cc(-c3cncs3)c(-c3cc4cncnc4s3)c21] |
| I-365′ | 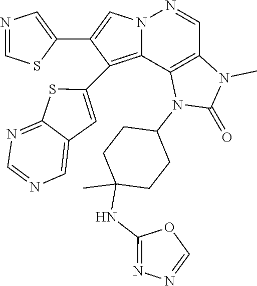 [SMILES: Cn1c(=O)n(C2CCC(C)(Nc3nnco3)CC2)c2c1cnn1cc(-c3cncs3)c(-c3cc4cncnc4s3)c21] |
| I-366 | 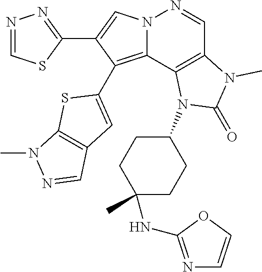 |
| I-366′ | 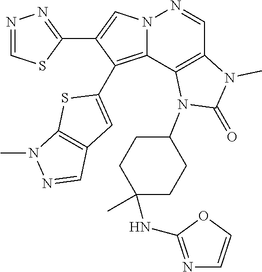 |
| I-367 | 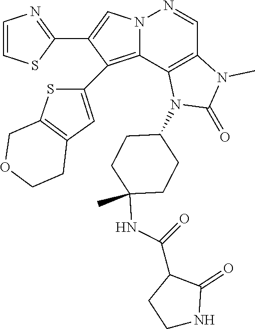 [SMILES: Cn1c(=O)n([C@H]2CC[C@@](C)(NC(=O)C3CCNC3=O)CC2)c2c1cnn1cc(-c3nccs3)c(-c3cc4c(s3)COCC4)c21] |
| I-367′ | 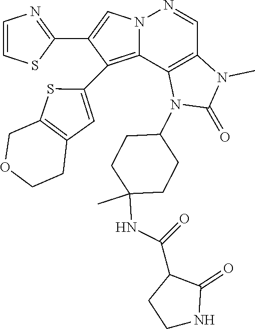 [SMILES: Cn1c(=O)n(C2CCC(C)(NC(=O)C3CCNC3=O)CC2)c2c1cnn1cc(-c3nccs3)c(-c3cc4c(s3)COCC4)c21] |
| I-368 | 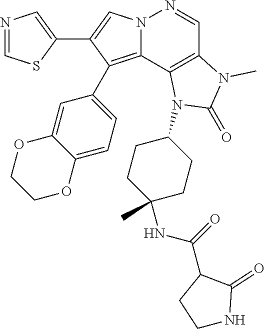 [SMILES: Cn1c(=O)n([C@H]2CC[C@@](C)(NC(=O)C3CCNC3=O)CC2)c2c1cnn1cc(-c3cncs3)c(-c3ccc4c(c3)OCCO4)c21] |
| I-368′ | 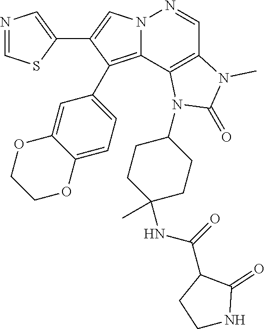 [SMILES: Cn1c(=O)n(C2CCC(C)(NC(=O)C3CCNC3=O)CC2)c2c1cnn1cc(-c3cncs3)c(-c3ccc4c(c3)OCCO4)c21] |
| I-369 | 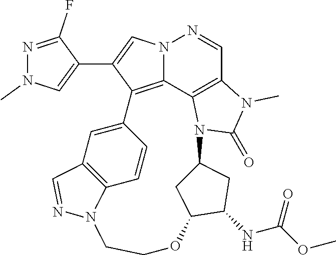 |
| I-369′ | 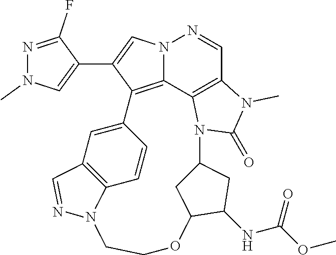 |
| I-370-1 | 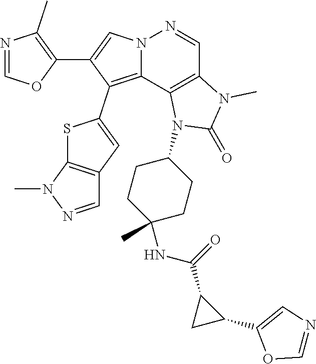 [SMILES: Cc1ncoc1-c1cn2ncc3c(c2c1-c1cc2cnn(C)c2s1)n([C@H]1CC[C@@](C)(NC(=O)[C@H]2C[C@H]2c2cnco2)CC1)c(=O)n3C] |
| I-370-2 | 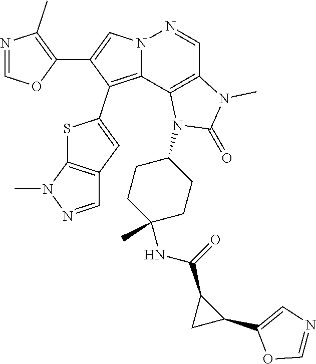 [SMILES: Cc1ncoc1-c1cn2ncc3c(c2c1-c1cc2cnn(C)c2s1)n([C@H]1CC[C@@](C)(NC(=O)[C@@H]2C[C@@H]2c2cnco2)CC1)c(=O)n3C] |
| I-370′ | 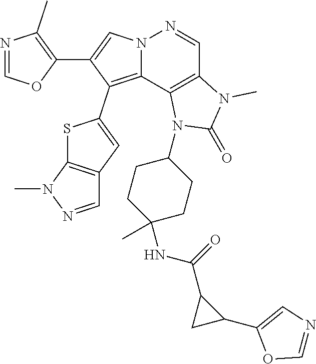 [SMILES: Cc1ncoc1-c1cn2ncc3c(c2c1-c1cc2cnn(C)c2s1)n(C1CCC(C)(NC(=O)C2CC2c2cnco2)CC1)c(=O)n3C] |
| I-371-1 | 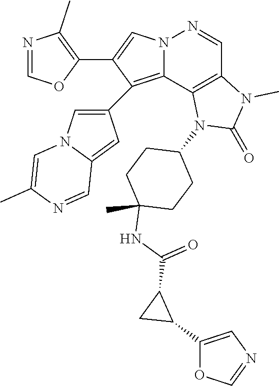 |
| I-371-2 | 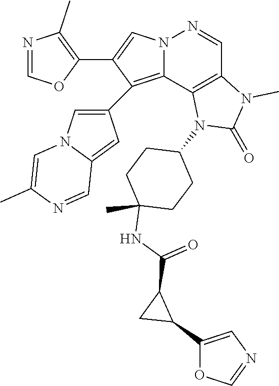 |
| I-371′ | 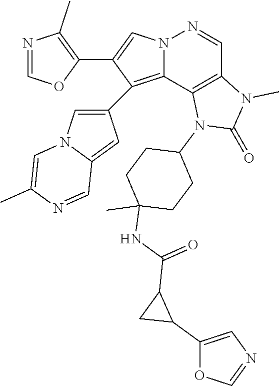 |
| I-372 | 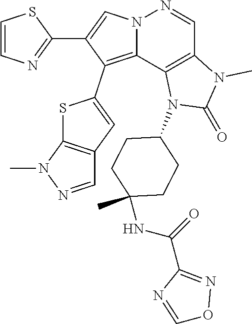 [SMILES: Cn1ncc2cc(-c3c(-c4nccs4)cn4ncc5c(c34)n([C@H]3CC[C@@](C)(NC(=O)c4ncon4)CC3)c(=O)n5C)sc21] |
| I-372′ | 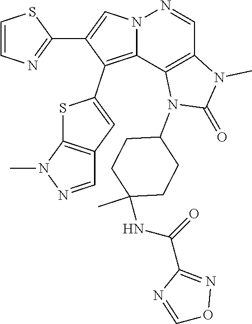 [SMILES: Cn1ncc2cc(-c3c(-c4nccs4)cn4ncc5c(c34)n(C3CCC(C)(NC(=O)c4ncon4)CC3)c(=O)n5C)sc21] |
| I-373 | 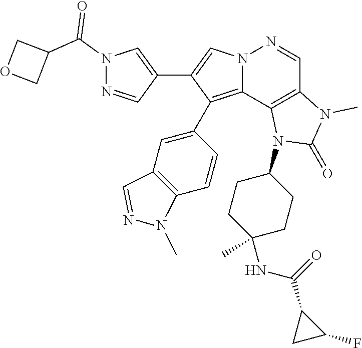 [SMILES: Cn1ncc2cc(-c3c(-c4cnn(C(=O)C5COC5)c4)cn4ncc5c(c34)n([C@H]3CC[C@@](C)(NC(=O)[C@H]4C[C@H]4F)CC3)c(=O)n5C)ccc21] |
| I-373′ | 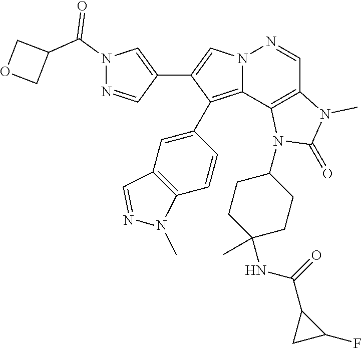 [SMILES: Cn1ncc2cc(-c3c(-c4cnn(C(=O)C5COC5)c4)cn4ncc5c(c34)n(C3CCC(C)(NC(=O)C4CC4F)CC3)c(=O)n5C)ccc21] |
| I-374 | 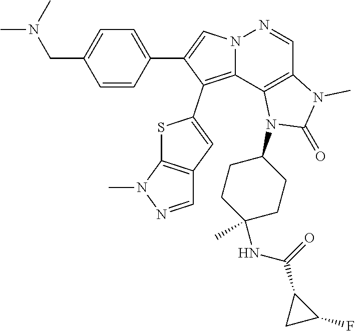 [SMILES: CN(C)Cc1ccc(-c2cn3ncc4c(c3c2-c2cc3cnn(C)c3s2)n([C@H]2CC[C@@](C)(NC(=O)[C@H]3C[C@H]3F)CC2)c(=O)n4C)cc1] |
| I-374′ | 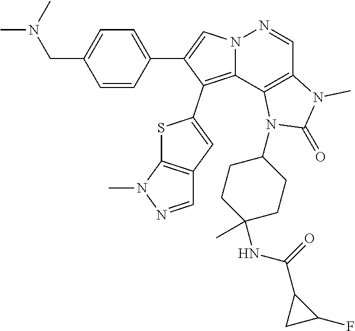 [SMILES: CN(C)Cc1ccc(-c2cn3ncc4c(c3c2-c2cc3cnn(C)c3s2)n(C2CCC(C)(NC(=O)C3CC3F)CC2)c(=O)n4C)cc1] |
| I-375 | 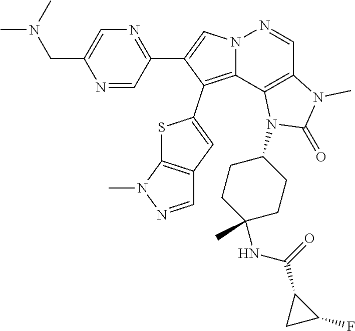 [SMILES: CN(C)Cc1cnc(-c2cn3ncc4c(c3c2-c2cc3cnn(C)c3s2)n([C@H]2CC[C@@](C)(NC(=O)[C@H]3C[C@H]3F)CC2)c(=O)n4C)cn1] |
| I-375′ | 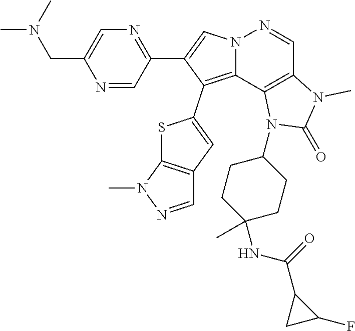 [SMILES: CN(C)Cc1cnc(-c2cn3ncc4c(c3c2-c2cc3cnn(C)c3s2)n(C2CCC(C)(NC(=O)C3CC3F)CC2)c(=O)n4C)cn1] |
| I-376 | 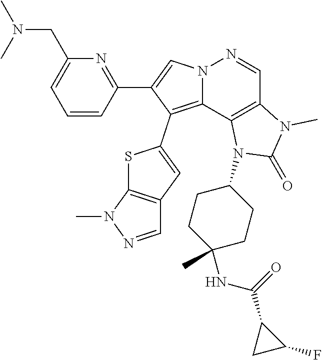 [SMILES: CN(C)Cc1cccc(-c2cn3ncc4c(c3c2-c2cc3cnn(C)c3s2)n([C@H]2CC[C@@](C)(NC(=O)[C@H]3C[C@H]3F)CC2)c(=O)n4C)n1] |
| I-376′ | 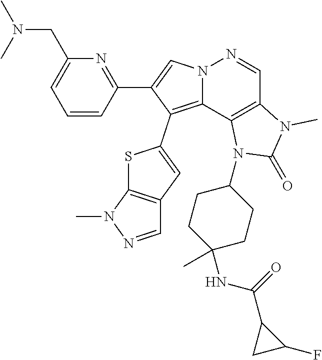 [SMILES: CN(C)Cc1cccc(-c2cn3ncc4c(c3c2-c2cc3cnn(C)c3s2)n(C2CCC(C)(NC(=O)C3CC3F)CC2)c(=O)n4C)n1] |
| I-377-1 | 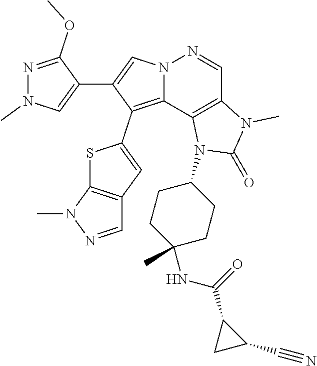 [SMILES: COc1nn(C)cc1-c1cn2ncc3c(c2c1-c1cc2cnn(C)c2s1)n([C@H]1CC[C@@](C)(NC(=O)[C@H]2C[C@H]2C#N)CC1)c(=O)n3C] |
| I-377-2 | 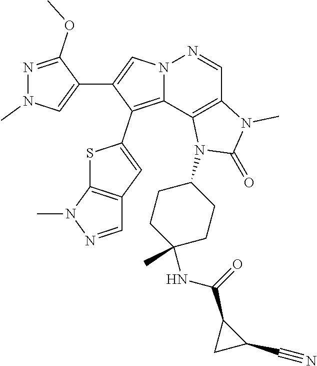 [SMILES: COc1nn(C)cc1-c1cn2ncc3c(c2c1-c1cc2cnn(C)c2s1)n([C@H]1CC[C@@](C)(NC(=O)[C@@H]2C[C@@H]2C#N)CC1)c(=O)n3C] |
| I-377′ | 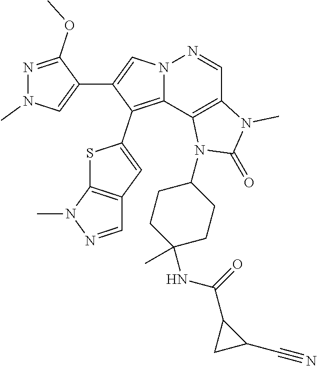 [SMILES: COc1nn(C)cc1-c1cn2ncc3c(c2c1-c1cc2cnn(C)c2s1)n(C1CCC(C)(NC(=O)C2CC2C#N)CC1)c(=O)n3C] |
| I-378 | 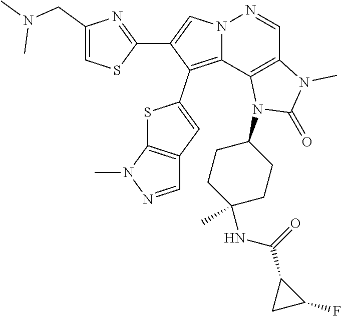 [SMILES: CN(C)Cc1csc(-c2cn3ncc4c(c3c2-c2cc3cnn(C)c3s2)n([C@H]2CC[C@@](C)(NC(=O)[C@H]3C[C@H]3F)CC2)c(=O)n4C)n1] |
| I-378′ | 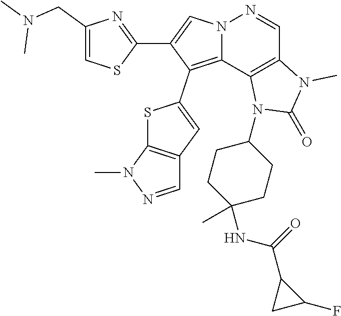 [SMILES: CN(C)Cc1csc(-c2cn3ncc4c(c3c2-c2cc3cnn(C)c3s2)n(C2CCC(C)(NC(=O)C3CC3F)CC2)c(=O)n4C)n1] |
| I-379 | 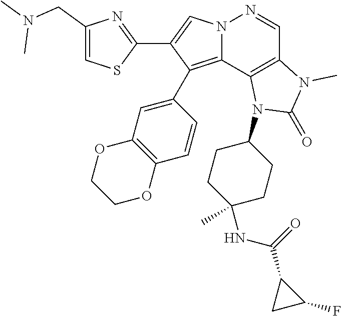 [SMILES: CN(C)Cc1csc(-c2cn3ncc4c(c3c2-c2ccc3c(c2)OCCO3)n([C@H]2CC[C@@](C)(NC(=O)[C@H]3C[C@H]3F)CC2)c(=O)n4C)n1] |
| I-379′ | 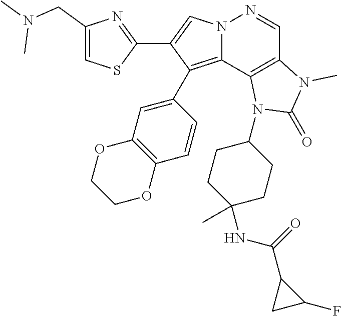 [SMILES: CN(C)Cc1csc(-c2cn3ncc4c(c3c2-c2ccc3c(c2)OCCO3)n(C2CCC(C)(NC(=O)C3CC3F)CC2)c(=O)n4C)n1] |
| I-380 | 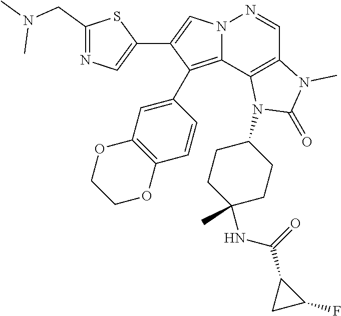 [SMILES: CN(C)Cc1ncc(-c2cn3ncc4c(c3c2-c2ccc3c(c2)OCCO3)n([C@H]2CC[C@@](C)(NC(=O)[C@H]3C[C@H]3F)CC2)c(=O)n4C)s1] |
| I-380′ | 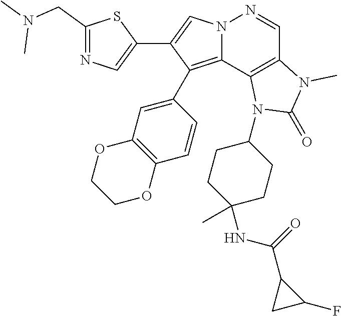 [SMILES: CN(C)Cc1ncc(-c2cn3ncc4c(c3c2-c2ccc3c(c2)OCCO3)n(C2CCC(C)(NC(=O)C3CC3F)CC2)c(=O)n4C)s1] |
| I-381 | 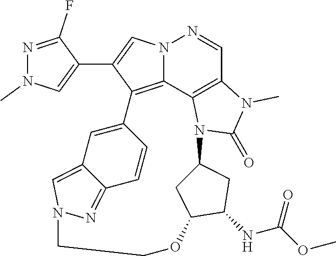 |
| I-381′ | 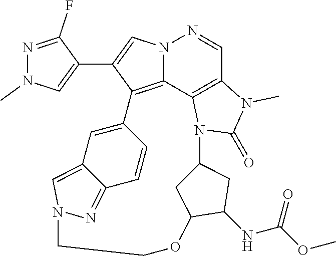 |

or a pharmaceutically acceptable salt thereof.

48. A pharmaceutical composition comprising the compound of claim 1, or a pharmaceutically acceptable salt thereof, and a pharmaceutically acceptable carrier.

49. A method of inhibiting JAK2 in a subject, comprising administering to the subject the compound of claim 1.

50. A method of treating a disease, disorder, or condition associated with JAK2, comprising administering to a subject in need thereof the compound of claim 1.

51. A method of treating cancer, comprising administering to a subject in need thereof the compound of claim 1.

52. A method of treating a hematological malignancy, comprising administering to a subject in need thereof the compound of claim 1.

53. (canceled)

54. A method of treating a myeloproliferative neoplasm, comprising administering to a subject in need thereof the compound of claim 1.

55. The method of claim 54, wherein the myeloproliferative neoplasm is polycythemia vera, essential thrombocytopenia, or myelofibrosis.
